(19) United States
(12) Patent Application Publication
(10) Pub. No.: US 2012/0277999 A1
(43) Pub. Date: Nov. 1, 2012

(54) METHODS, KITS AND ARRAYS FOR SCREENING FOR, PREDICTING AND IDENTIFYING DONORS FOR HEMATOPOIETIC CELL TRANSPLANTATION, AND PREDICTING RISK OF HEMATOPOIETIC CELL TRANSPLANT (HCT) TO INDUCE GRAFT VS. HOST DISEASE (GVHD)

(75) Inventors: Roland Somogyi, Sydenham (CA); Larry David Greller, Annapolis, MD (US)

(73) Assignee: PBD BIODIAGNOSTICS, LLC, Davidsonville, MD (US)

(21) Appl. No.: 13/286,058

(22) Filed: Oct. 31, 2011

Related U.S. Application Data
(60) Provisional application No. 61/498,965, filed on Jun. 20, 2011.
(60) Provisional application No. 61/408,491, filed on Oct. 29, 2010.

(51) Int. Cl. G06F 19/24; C40B 40/06; G06F 17/30; C07H 21/04

(52) U.S. Cl. 702/19; 536/24.33; 536/24.31; 506/16; 707/736; 707/E17.045

(57) ABSTRACT

The invention relates to predicting or determining risk of a hematopoietic cell transplant (HCT) from a donor to induce Graft vs. Host Disease (GVHD) in a HCT recipient; to classifying HCT from a candidate donor according to the risk of inducing GVHD in a HCT recipient; and to organizational constructs (e.g., databases) and methods of producing organizational constructs (e.g., databases) in which HCT of one or more candidate donors is classified or scored according to risk of inducing GVHD in a HCT recipient. The invention also relates to kits and arrays useful for predicting or determining risk of HCT from a candidate donor to induce GVHD in a HCT recipient, and for classifying or scoring such donors according to risk of inducing GVHD in a HCT recipient.

### RELATED APPLICATIONS

This application claims the benefit of priority of application Ser. No. 61/498,965, filed Jun. 20, 2011, and application Ser. No. 61/408,491, filed Oct. 29, 2010, all of which applications are expressly incorporated herein by reference in their entirety.

### TECHNICAL FIELD

The invention relates to predicting or determining risk of a hematopoietic cell transplant (HCT) from a donor to induce Graft vs. Host Disease (GVHD) in a HCT recipient. The invention also relates to classifying HCT from a candidate donor according to the risk of inducing GVHD in a HCT recipient. The invention further relates to organizational constructs (e.g., databases) and methods of producing organizational constructs (e.g., databases) in which HCT of one or more candidate donors is classified or scored according to risk of inducing GVHD in a HCT recipient. The invention moreover relates to kits and arrays useful for predicting or determining risk of HCT from a candidate donor to induce GVHD in a HCT recipient, and for classifiying or scoring such donors according to risk of inducing GVHD in a HCT recipient.

### INTRODUCTION

Hematopoietic cell transplantation (HCT, also referred to herein as Hematopoietic cell transplant) [the more modem term], or bone marrow transplantation (BMT) [the more lay term], is an often life-extending or curative treatment for a variety of different hematologic cancers and diseases, such as acute lymphoblastic leukemia, acute myeloid leukemia, chronic myelogenous leukemia, chronic lymphocytic leukemia, myelodysplastic syndrome (ALL, AML, CML, CLL, and MDS, respectively). The major obstacle to more widespread and successful application of HCT is the risk of GVHD (Graft vs. Host Disease) in a HCT recipient.

Of the 10,000 HCTs annually in the U.S. (conservatively, more precise number is closer to over 12,000 annually), a large majority (˜75%) is carried out using donors familially unrelated to the HCT recipients. It is well-established in the medical practice of HCT that on average only 1 of 4 candidates for HCT will ever have a sibling, or other family relative, suitable as a donor, which is why approximately 3 out of 4 HCTs that occur in the US and in much of Europe involve donors familially unrelated to corresponding patients. Of these ˜7,500 unrelated donor transplantations, ˜5,600 (−75%) use donor\recipient pairs HLA-matched for so-called 10/10 major alleles (standard nomenclature: HLA-A, HLA-B, HLA-C, HLA-DRB1, HLA-DQB1).

Graft vs. Host Disease (GVHD) can be a severe and fatal rejection of the HCT recipient's tissues and organs (the host) by the immune system T-cells originating from the donor's transplanted hematopoietic stem cells (the graft) (Bhushan & Collins 2003; Ferrara, et al., 2005). Even with close HLA (human leukocyte antigen) matching between HCT donors and HCT recipients for 10/10 alleles (HLA-A, HLA-B, HLA-C, HLA-DRB1, HLA-DQB1), GVHD occurs in 50% to 60% of transplant recipients, whether using either sibling or familially unrelated donors. Accordingly, there is a need for predicting and determining risk of a hematopoietic cell transplant (HCT) from a donor to induce GVHD in a HCT recipient, and identifying donors at lower risk for inducing GVHD to reduce GVHD in a HCT recipient. The invention herein satisfies this need and provides additional benefits.

### SUMMARY

The invention is based, at least in part on analysis of samples from 180HCTs carried out in 57 different U.S. transplant centers, using donors unrelated to the respective recipient (i.e., patient). Gene expression analysis revealed molecular RNA marker profiles in peripheral blood-derived pre-transplant donor CD4+ T cells that are highly predictive of acute or chronic GVHD outcomes in the HCT recipient. Overall, the data reveals for several multi-gene predictive models, using various gene marker combinations, covering outcome prediction of different degrees of acute and chronic GVHD (see Table 20), (1) Negative Predictive Values (the fraction of HCTs that are predicted as GVHD-negative, which are predicted correctly) of 82% on average over all GNOS (GVHD Negative Outcome Score) thresholds, and 78% on average for GNOS threshold of 0.50, (2) Specificities (True Negative Rate, i.e. the fraction of GVHD-negative HCTs that are correctly predicted as GVHD-negative) of 50% on average over all GNOS thresholds, and 78% on average for GNOS threshold of 0.50, and (3) Sensitivities (True Positive Rate, i.e. the fraction of GVHD-positive HCTs that are correctly predicted as GVHD-positive) of 88% on average over all GNOS thresholds, and 78% on average for GNOS threshold of 0.50. In particular, for one of the best performing multi-gene predictive models, SG43RGP36-RGPgreedysearch, for the Gneg vs. Gag3 division (no GVHD vs. acute grades BI or IV GVHD), at GNOS threshold of 0.55, the observed Negative Predictive Value is 92%, Specificity is 80%, and Sensitivity is 94% (see Table 20). The accurate, donor-based, pre-transplant GVHD outcome prediction is robust with respect to variations of transplant clinical center sample origin, the hematological disease outcome classification by physicians and whether the donor HCT was in the form of bone marrow or PBMCs (peripheral blood mononuclear cells). Reliably predicting GVHD from donor T-cell RNA expression measurements in donors familially unrelated and related to HCT recipients, optionally as an additional practice to HLA matching, and selecting low GVHD-risk donor HCT, would significantly reduce the occurrence and intensity/severity of GVHD in HCT recipients.

Thus, in accordance with the invention there are provided methods for predicting or determining the risk of a hematopoietic cell transplant (HCT) from an actual or a candidate donor to induce (or not) graft vs. host disease (GVHD) in a HCT recipient. In one embodiment, a method includes measuring expression of one or more positive or negative GVHD predictor genes, or a combination of positive and/or negative GVHD predictor genes, selected from Tables 1 (RNA 1538), 2, 2A (RNA 143), 2B (RNA 192), 3, 13 (SG175), 15 (SG128) or 18 (SG64), or a polymorphism thereof, in CD4+ T cells or CD8+ T cells from a candidate donor. An expression value for the positive or negative GVHD predictor genes based upon the gene expression level measured is obtained. Alternatively, or in addition, linear or non-linear combinations of expression values for the combination of positive and/or negative GVHD predictor genes based upon the expression levels measured is obtained. A comparison is performed, of the expression value for the positive or negative GVHD predictor gene to a predefined reference expression value for the positive or negative GVHD predictor gene, or of the linear or non-linear combinations of expression values for the combination of positive and/or negative GVHD predictor genes to predefined reference values for the linear or non-linear combinations of the positive and/or negative GVHD predictor genes. Based upon the comparison, 1) an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, 2) an expression value for the negative GVHD predictor gene greater or less than the predefined reference expression value for the negative GVHD predictor gene indicates that the HCT from the candidate donor is at lower or higher risk, respectively, of inducing GVHD in a HCT recipient, 3) a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient, and 4) a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient. Based upon an evaluation of expression values comparisons, total numbers or identity of positive or negative GVHD predictor genes, or comparisons of the linear or non linear combination of expression values for the combination of positive and/or negative GVHD predictor genes, that indicate that the HCT from the candidate donor is at higher or lower risk of inducing GVHD in a HCT recipient, the risk or probability of the HCT from the candidate donor to induce or to not induce graft vs. host disease (GVHD) in a HCT recipient is predicted and/or determined.

In accordance with the invention, there are also provided methods for predicting or determining the risk of HCT from an actual or candidate donor to induce (or not) graft vs. host disease (GVHD) in a HCT recipient. In one embodiment, a method includes contacting CD4+ T cells or CD8+ T cells, or nucleic acid or protein expressed by CD4+ T cells or CD8+ T cells, from a candidate donor with an analyte that detects expression of one or more positive or negative GVHD predictor genes listed in Tables 1 (RNA 1538), 2, 2A (RNA 143), 2B (RNA 192), 3, 13 (SG175), 15 (SG128) or 18 (SG64), or a polymorphism thereof, and measuring expression of the one or more positive or negative GVHD predictor genes in CD4+ T cells or CD8+ T cells to obtain an expression value for the positive or negative GVHD predictor genes, or measuring expression of a combination of the positive and/or negative GVHD predictor genes to obtain linear or non-linear combinations of expression values for the combination of positive and/or negative GVHD predictor genes. A comparison is performed, of the expression value for the positive or negative GVHD predictor gene to a predefined reference expression value for the positive or negative GVHD predictor gene, or of the linear or non-linear combinations of expression values of the combination of positive and/or negative GVHD predictor genes to a predefined reference value for the linear or non-linear combinations of expression values of the combination of positive and/or negative GVHD predictor genes. Based upon the comparison, 1) an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, 2) an expression value for the negative GVHD predictor gene greater or less than the reference expression value for the negative GVHD predictor gene indicates that the HCT from the candidate donor is at lower or higher risk, respectively, of inducing GVHD in a HCT recipient, 3) a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient, and 4) a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient. Based upon an evaluation of expression value comparisons, total numbers or identity of positive or negative GVHD predictor genes, or linear or non linear combinations of expression values of the combination of positive and/or negative GVHD predictor gene comparisons, that indicate that the HCT from the candidate donor is at higher or lower risk of inducing GVHD in a HCT recipient, leads to predicting or determining the risk of the HCT from the candidate donor to induce or to not induce GVHD in a HCT recipient.

In accordance with the invention, there are further provided methods for classifying a hematopoietic cell transplant (HCT) from an actual or a candidate donor for risk of inducing (or not) graft vs. host disease (GVHD) in a HCT recipient. In one embodiment, a method includes measuring expression of a plurality of positive or negative GVHD predictor genes selected from a gene listed in Tables 1 (RNA 1538), 2, 2A (RNA 143), 2B (RNA 192), 3, 13 (SG175), 15 (SG128) or 18 (SG64), or a polymorphism thereof, in CD4+ T cells or CD8+ T cells from the candidate HCT donor, and obtaining an expression value for the positive or negative GVHD predictor genes based upon the expression measured, or obtaining linear or non-linear combinations of expression values for the combination of positive and/or negative GVHD predictor genes based upon the expression measured. A comparison is performed, of the expression value for the positive or negative GVHD predictor gene to a predefined reference expression value for the positive or negative GVHD predictor gene, or of the linear or non-linear combinations of expression values for the combination of positive and/or negative GVHD predictor genes to predefined reference values for the linear or non-linear combinations of the positive and/or negative GVHD predictor genes. Based upon the comparison, 1) an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, 2) an expression value for the negative GVHD predictor gene greater or less than the reference expression value for the negative GVHD predictor gene indicates that the HCT from the candidate donor is at lower or higher risk, respectively, of inducing GVHD in a HCT recipient, 3) a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient, and 4) a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient. The actual or candidate donor HCT is classified for risk of inducing or not inducing graft vs. host disease (GVHD) based upon an evaluation of expression values, total numbers or identity of positive or negative GVHD predictor genes, or combination of positive and/or negative GVHD predictor genes, that indicate that the HCT from the candidate donor is at higher or lower risk of inducing GVHD in a HCT recipient.

In accordance with the invention, there are moreover provided methods for producing a database or organizational construct comprising a plurality of actual or candidate HCT donors each assigned a score (or classified) based upon the probability or degree of risk of the actual or candidate donor HCT to induce or not to induce graft vs. host disease (GVHD) in a HCT recipient. In one embodiment, a method includes measuring expression of one or more positive or negative GVHD predictor genes listed in Tables 1 (RNA 1538), 2, 2A (RNA 143), 2B (RNA 192), 3, 13 (SG175), 15 (SG128) or 18 (SG64), or a polymorphism thereof, in CD4+ T cells or CD8+ T cells from an actual or a candidate donor, and obtaining an expression value for the positive or negative GVHD predictor genes based upon the expression measured, or obtaining linear or non-linear combinations of expression values for the combination of positive and/or negative GVHD predictor genes based upon the expression measured. A comparison is performed, of the expression value for the positive or negative GVHD predictor gene to a predefined reference expression value for the positive or negative GVHD predictor gene, or of the linear or non-linear combinations of expression values for the combination of positive and/or negative GVHD predictor genes to predefined reference values for the linear or non-linear combinations of the positive and/or negative GVHD predictor genes. Based upon the comparison, 1) an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, 2) an expression value for the negative GVHD predictor gene greater or less than the reference expression value for the negative GVHD predictor gene indicates that the HCT from the candidate donor is at lower or higher risk, respectively, of inducing GVHD in a HCT recipient, 3) a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient, and 4) a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient. The actual or candidate donor HCT is assigned a score or classified based upon an evaluation of expression value comparisons, total numbers or identity of positive or negative GVHD predictor genes, or linear or non linear combinations of expression values of the combination of positive and/or negative GVHD predictor gene comparisons, that indicate that the HCT from the candidate donor is at higher or lower risk of inducing GVHD in a HCT recipient, wherein the score reflects the probability or degree of risk of the actual or candidate donor HCT to induce GVHD in a HCT recipient. The score can then be recorded or stored. Subsequently, the foregoing steps can be repeated for one or more additional actual or candidate HCT donors, thereby producing a database or organizational construct comprising actual or candidate HCT donors each assigned a score based upon the probability or degree of risk of the actual or candidate donor HCT to induce or to not induce graft vs. host disease (GVHD) in a HCT recipient.

Exemplary positive and negative “GVHD” predictor genes and exemplary housekeeping (“HSK”) genes for measurement, are listed in and can be selected from Tables 1 (RNA 1538, SEQ ID NOs:1-1546), 2, 2A (RNA143), 2B (RNA192), 3, 12 (HSK6), 13 (SG175), 15 (SG128) and 18 (SG64). The sequences of 1546, 192, 175, 128 and 64 exemplary positive and negative GVHD predictor genes and HSK (housekeeping genes) are listed as a “Sequence Listing Appendix” following the claims (SEQ ID NOs:1-1738). Exemplary probes and primers for hybridization (detection) and/or RT-PCR which can be used to detect, measure or analyze expression of the positive and negative predictor genes are also listed, or can be derived from or based upon, for example, sequences listed in Tables 1 (RNA 1538, SEQ ID NOs:1-1546), 2, 2A (RNA143), 2B (RNA192), 3, 12 (HSK6), 13 (SG175), 15 (SG128) and 18 (SG64).

In accordance with the invention, there are additionally provided databases and organizational constructs. In one embodiment, a database or organizational construct includes a gene expression profile of two or more positive or negative GVHD predictor genes, linear or non-linear combinations of expression values for combinations of positive and/or negative GVHD predictor genes, or scores or risk probability of inducing or not inducing GVHD, from a plurality of actual or candidate HCT donors, wherein the two or more positive or negative GVHD predictor genes are any combination of genes listed in Tables 1 (RNA 1538, SEQ ID NOs:1-1546), 2, 2A (RNA143), 2B (RNA192), 3, 12 (HSK6), 13 (SG175), 15 (SG128) and 18 (SG64), or a polymorphism thereof, or wherein the scores or risk probability is based upon expression of one or more positive or negative GVHD predictor genes listed in Tables 1 (RNA 1538, SEQ ID NOs:1-1546), 2, 2A (RNA143), 2B (RNA192), 3, 12 (HSK6), 13 (SG175), 15 (SG128) and 18 (SG64), and wherein the database or organizational construct associates the gene expression profile, score or risk probability of inducing or not inducing GVHD, with each of the actual or candidate HCT donors.

In accordance with the invention, there are yet further provided kits. In one embodiment, a kit includes one or more analytes for detecting, measuring or analyzing one or more positive and/or negative GVHD predictor genes. In a particular aspect, a kit includes two or more primer pairs, wherein each primer pair is oppositely oriented to each other, and wherein each of the primer pairs hybridize to RNA or cDNA produced from one of the positive or negative predictor genes listed in Tables 1 (RNA 1538, SEQ ID NOs:1-1546), 2, 2A (RNA143), 2B (RNA192), 3, 12 (HSK6), 13 (SG175), 15 (SG128) or 18 (SG64), or a polymorphism thereof. In another particular aspect, a kit includes one or more nucleic acid probes, wherein at least one of said one or more probes hybridizes to RNA or cDNA of one or more of the positive or negative GVHD predictor genes listed in Tables 1 (RNA 1538, SEQ ID NOs:1-1546), 2, 2A (RNA143), 2B (RNA192), 3, 12 (HSK6), 13 (SG175), 15 (SG128) or 18 (SG64), or a polymorphism thereof.

In accordance with the invention, there are still further provided arrays. In one embodiment, an array includes one or more analytes for detecting, measuring or analyzing one or more positive and/or negative GVHD predictor genes. In a particular aspect, an array includes two or more primer pairs, wherein each primer pair is oppositely oriented to each other, wherein each of the primer pairs hybridize to RNA or cDNA produced from one of the positive or negative GVHD predictor genes listed in Tables 1 (RNA 1538, SEQ ID NOs:1-1546), 2, 2A (RNA143), 2B (RNA192), 3, 12 (HSK6), 13 (SG175), 15 (SG128) or 18 (SG64) or a polymorphism thereof, and wherein each primer pair is affixed to or contained in a support or substrate. In another particular aspect, an array includes one or more probes, wherein at least one of the probes hybridizes to RNA or cDNA produced from a positive or negative GVHD predictor gene listed in Tables 1 (RNA 1538, SEQ ID NOs:1-1546), 2, 2A (RNA143), 2B (RNA192), 3, 12 (HSK6), 13 (SG175), 15 (SG128) or 18 (SG64), or a polymorphism thereof, and wherein each probe is affixed to or contained in a support or substrate.

### DESCRIPTION OF DRAWINGS

FIG. 1 shows a representative unsmoothed histogram of 48803 probes by 48 samples Illumina signal values (Plot 5,1).

FIG. 2 shows a scatterplot of log 10(bead_stderr) vs. log 10(positive signal) from Illumina measurements.

FIG. 3 shows empirically that the vast majority of Illumina raw signal data occurs at levels less than about 1500 even though there are many signals at the multiple tens of thousands level; that for 98% of signals there is still clear and marked dependence of standard deviation or variance with signal level; and is the data employed in the calculation of the VST data-dependent parameters c1 and c2 for each sample separately.

FIG. 4 shows a histogram of all the signal values of the 48803 by 48 sample ensemble after the ensemble is transformed using VST. The largest 5% are omitted to improve visualization along the horizontal axis.

FIG. 5 shows RNA expression measurement values plotted for all 122 samples in ascending order for each of the six GVHD outcome classes, and labeled according to the samples' transplant center sources (TCS) for CTCF. BLVRA TCS, RNA20 TCS)

FIG. 6 shows RNA expression measurement values plotted for all 122 samples in ascending order for each of the six GVHD outcome classes, and labeled according to the samples' transplant center sources (TCS) for BLVRA.)

FIG. 7 shows RNA expression measurement values plotted for all 122 samples in ascending order for each of the six GVHD outcome classes, and labeled according to the samples' transplant center sources (TCS) for the RNA20 model gene set.

FIG. 8 shows a steady, monotonously increasing series of GVHD Group average with GVHD Group number for CTCF.

FIG. 9 shows a steady downward trend of GVHD Group average with GVHD Group number for BLVRA.

FIG. 10 is a plot (RNA20 GROUPS) of the relative score of GVHD negative votes from 20 well-performing individual LDA genes, and shows a steady downward trend of GVHD Group average score with increasing GVHD severity.

FIG. 11 shows sample-specific GVHD outcome prediction for anyGVHD vs. no GVHD for the LDA model corresponding to the individual RNA expression marker, CTCF. CTCF LDA samples are classified as GVHD negative below the separatrix.

FIG. 12 shows sample-specific GVHD outcome prediction for anyGVHD vs. no GVHD for the LDA models corresponding to the individual RNA expression marker, BLVRA. BLVRA LDA samples are classified as GVHD negative above the separatrix.

FIG. 13 shows sample-specific GVHD outcome prediction for anyGVHD vs. no GVHD for the LDA models corresponding to the 20 RNA marker voting model, RNA20 LDA-A). RNA 20 LDA samples are classified as GVHD negative above the separatrix.

FIG. 14 shows that in distinguishing chronic GVHD (alone or in combination with any form of acute GVHD) from no GVHD outcomes (cGVHD vs. noGVHD), only 2 False Negative classifications were reported (RNA20 LDA-B) (negative predictive value=0.95).

FIG. 15 shows that in distinguishing any form of acute GVHD (alone or in combination with chronic GVHD) from no GVHD outcomes (aGVHD vs. noGVHD), only 3 False Negative classifications were reported (RNA20 LDA-C) (negative predictive value=0.94).

FIG. 16 shows that in distinguishing chronic GVHD in combination with acute GVHD (in any form) from no GVHD outcomes (a&cGVHD vs. noGVHD), only 1 False Negative classification was reported (RNA20 LDA-D) (Negative Predictive Value=0.96).

FIG. 17 shows that in distinguishing the most severe forms of grade 3 or 4 acute GVHD (alone or in combination with chronic GVHD) from no GVHD outcomes (a34GVHD vs. noGVHD), not a single False Negative classification was reported (RNA20 LDA-E) (Negative Predictive Value=1.00).

FIG. 18 shows selection of a threshold value of 0.77 to minimize False Negatives and maximize the Negative Predictive Value, while maintaining a relatively high number of True Negatives and high true negative rate (RNA20 LDA PERFORMANCE-A, for any GVHD vs. no GVHD).

FIG. 19 shows the detailed behavior of all 5 LDA accuracy measures, also including Positive Predictive Value (PPV) and True Positive Rate (TPR, Sensitivity), RNA20 LDA PERFORMANCE-B, for any GVHD vs. no GVHD.

FIG. 20 shows a comparison of Vmod T-test performance in the presence of noise, ranging from 0.1× to 10× of SG measurement standard deviation, for the Gneg vs. Gag3 division (“3VmodnoisecompTtest”), at a GNOS threshold of 0.55 and prevalence P=0.25 (average and s.d. of performance values over 1,000 iterations of noise).

FIG. 21 shows a comparison of Vmod projected GVHD reduction in the presence of noise, ranging from 0.1× to 10× of SG measurement standard deviation, for the Gneg vs. Gag3 division (:3VmodnoisecompGVHDred″), at a GNOS threshold of 0.55 and prevalence P=0.25 (average and s.d. of performance values over 1,000 iterations of noise).

### DETAILED DESCRIPTION

The invention relates to gene expression profiles of CD4+ T cells from AHCT (allogeneic hematopoietic cell transplantation, or hematopoietic cell transplant) donors, such donors known to induce GVHD and known not to induce GVHD in a HCT recipient. The studies described herein identify numerous genes in CD4+ of HCT donors whose expression was increased in HCT donors that did not induce GVHD in HCT recipients, referred to as negative predictor genes. The studies described herein also identify numerous genes in CD4+ T cells of HCT donors whose expression was increased in HCT donors that did induce GVHD in HCT recipients, referred to as positive predictor genes. Measuring expression of one or more such “GVHD” predictor genes can be used to ascertain or predict the risk of HCT from a candidate donor to induce GVHD in an HCT recipient. For example, expression of one or more such genes in CD4+ T cells of candidate donor HCT, optionally HLA matched (10 out of 10, or 9 out of 10, HLA matches), to an HCT recipient can be measured. Increased expression of one or more genes known to increase with HCT inducing GVHD in a HCT recipient can provide information as to whether the donor HCT is likely to induce GVHD in a HCT recipient. Likewise, increased expression of one or more genes known to increase with HCT not inducing GVHD in a HCT recipient can provide information as to whether the donor is likely to not induce GVHD in a HCT recipient. Measurement of one or more such positive or negative GVHD predictor genes, or such positive or negative GVHD predictor genes in a combination, a plurality of positive and negative GVHD predictor genes, or particularly ratios of such positive and/or negative GVHD predictor genes, can be used to predict or determine the risk of any HCT donor of inducing GVHD in a HCT recipient, with a moderate, high or very high degree of confidence.

Accordingly, the invention provides methods for predicting and/or determining the risk of a hematopoietic cell transplant (HCT) from a candidate donor to induce or not induce graft vs. host disease (GVHD) in a HCT recipient. In one embodiment, a method includes In one embodiment, a method includes measuring expression of one or more positive or negative GVHD predictor genes, or a combination of positive and/or negative GVHD predictor genes, selected from Tables 1 (RNA 1538), 2, 2A (RNA 143), 2B (RNA 192), 3, 13 (SG175), 15 (SG128) or 18 (SG64), or a polymorphism thereof, in CD4+ T cells or CD8+ T cells from a candidate donor. An expression value for the positive or negative GVHD predictor genes based upon the gene expression level measured is obtained, or a linear or non-linear combinations of expression values for the combination of positive and/or negative GVHD predictor genes based upon the expression levels measured is obtained. A comparison of the expression value for the positive or negative GVHD predictor gene to a predefined reference expression value for the positive or negative GVHD predictor gene, or of the linear or non-linear combinations of expression values for the combination of positive and/or negative GVHD predictor genes to predefined reference values for the linear or non-linear combinations of the positive and/or negative GVHD predictor genes is performed. A comparison in which 1) an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, 2) an expression value for the negative GVHD predictor gene greater or less than the predefined reference expression value for the negative GVHD predictor gene indicates that the HCT from the candidate donor is at lower or higher risk, respectively, of inducing GVHD in a HCT recipient, 3) a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient, and 4) a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient. Based upon an evaluation of expression values comparisons, total numbers or identity of positive or negative GVHD predictor genes, or comparisions of the linear or non linear combination of expression values for the combination of positive and/or negative GVHD predictor genes, that indicate that the HCT from the candidate donor is at higher or lower risk of inducing GVHD in a HCT recipient, the risk or probability of the HCT from the candidate donor to induce or to not induce graft vs. host disease (GVHD) in a HCT recipient is predicted and/or determined.

In another embodiment, a method for predicting and/or determining the risk of a hematopoietic cell transplant (HCT) from a candidate donor to induce or not induce graft vs. host disease (GVHD) in a HCT recipient includes contacting CD4+ T cells or CD8+ T cells, or nucleic acid or protein expressed by CD4+ T cells or CD8+ T cells, from a candidate donor with an analyte that detects expression of one or more positive or negative GVHD predictor genes listed in Tables 1 (RNA 1538), 2, 2A (RNA 143), 2B (RNA 192), 3, 13 (SG175), 15 (SG128) or 18 (SG64), or a polymorphism thereof, and measuring expression of the one or more positive or negative GVHD predictor genes in CD4+ T cells or CD8+ T cells to obtain an expression value for the positive or negative GVHD predictor genes, or measuring expression of a combination of the positive and/or negative GVHD predictor genes to obtain linear or non-linear combinations of expression values for the combination of positive and/or negative GVHD predictor genes. A comparison of the expression value for the positive or negative GVHD predictor gene to a predefined reference expression value for the positive or negative GVHD predictor gene, or of the linear or non-linear combinations of expression values of the combination of positive and/or negative GVHD predictor genes to a predefined reference value for the linear or non-linear combinations of expression values of the combination of positive and/or negative GVHD predictor genes, is performed. Based upon the comparison, 1) an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, 2) an expression value for the negative GVHD predictor gene greater or less than the reference expression value for the negative GVHD predictor gene indicates that the HCT from the candidate donor is at lower or higher risk, respectively, of inducing GVHD in a HCT recipient, 3) a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient, and 4) a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient. Based upon an evaluation of expression value comparisons, total numbers or identity of positive or negative GVHD predictor genes, or linear or non linear combinations of expression values of the combination of positive and/or negative GVHD predictor gene comparisons, that indicate that the HCT from the candidate donor is at higher or lower risk of inducing GVHD in a HCT recipient, leads to predicting or determining the risk of the HCT from the candidate donor to induce or to not induce GVHD in a HCT recipient.

The invention also provides methods for classifying or categorizing a candidate hematopoietic cell transplant (HCT) donor according to the risk or probability of inducing or not inducing graft vs. host disease (GVHD) in a HCT recipient. In one embodiment, a method includes measuring expression of a plurality of positive or negative GVHD predictor genes selected from a gene listed in Tables 1 (RNA 1538), 2, 2A (RNA 143), 2B (RNA 192), 3, 13 (SG175), 15 (SG128) or 18 (SG64), or a polymorphism thereof, in CD4+ T cells or CD8+ T cells from the candidate HCT donor, and obtaining an expression value for the positive or negative GVHD predictor genes based upon the expression measured, or obtaining linear or non-linear combinations of expression values for the combination of positive and/or negative GVHD predictor genes based upon the expression measured. A comparison of the expression value for the positive or negative GVHD predictor gene to a predefined reference expression value for the positive or negative GVHD predictor gene, or of the linear or non-linear combinations of expression values for the combination of positive and/or negative GVHD predictor genes to predefined reference values for the linear or non-linear combinations of the positive and/or negative GVHD predictor genes, is performed. Based upon the comparison, 1) an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, 2) an expression value for the negative GVHD predictor gene greater or less than the reference expression value for the negative GVHD predictor gene indicates that the HCT from the candidate donor is at lower or higher risk, respectively, of inducing GVHD in a HCT recipient, 3) a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient, and 4) a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient. The actual or candidate donor HCT is classified for risk of inducing or not inducing graft vs. host disease (GVHD) based upon an evaluation of expression value comparisons, total numbers or identity of positive or negative GVHD predictor genes, or linear or non linear combinations of expression values of the combination of positive and/or negative GVHD predictor gene comparisons, that indicate that the HCT from the candidate donor is at higher or lower risk of inducing GVHD in a HCT recipient.

The invention further provides methods for producing or generating databases and organizational constructs, in which the database or organizational construct includes a plurality of actual and/or candidate HCT donors, optionally classified, categorized or assigned a score or identified based upon the probability or degree of risk of HCT from the actual or candidate donor to induce or to not induce graft vs. host disease (GVHD) in a HCT recipient. In one embodiment, a method includes: measuring expression of one or more positive or negative GVHD predictor genes listed in Tables 1 (RNA 1538), 2, 2A (RNA 143), 2B (RNA 192), 3, 13 (SG175), 15 (SG128) or 18 (SG64), or a polymorphism thereof, in CD4+ T cells or CD8+ T cells from an actual or a candidate donor, and obtaining an expression value for the positive or negative GVHD predictor genes based upon the expression measured, or obtaining linear or non-linear combinations of expression values for the combination of positive and/or negative GVHD predictor genes based upon the expression measured. A comparison is performed, of the expression value for the positive or negative GVHD predictor gene to a predefined reference expression value for the positive or negative GVHD predictor gene, or of the linear or non-linear combinations of expression values for the combination of positive and/or negative GVHD predictor genes to predefined reference values for the linear or non-linear combinations of the positive and/or negative GVHD predictor genes. Based upon the comparison, 1) an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, 2) an expression value for the negative GVHD predictor gene greater or less than the reference expression value for the negative GVHD predictor gene indicates that the HCT from the candidate donor is at lower or higher risk, respectively, of inducing GVHD in a HCT recipient, 3) a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient, and 4) a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient. The actual or candidate donor HCT is assigned a score or classified based upon an evaluation of expression value comparisons, total numbers or identity of positive or negative GVHD predictor genes, or linear or non linear combinations of expression values of the combination of positive and/or negative GVHD predictor gene comparisons, that indicate that the HCT from the candidate donor is at higher or lower risk of inducing GVHD in a HCT recipient, wherein the score reflects the probability or degree of risk of the actual or candidate donor HCT to induce GVHD in a HCT recipient. The score can then be recorded or stored, and the foregoing steps can optionally be repeated for one or more additional actual or candidate HCT donors, to produce a database or organizational construct comprising actual or candidate HCT donors each assigned a score based upon the probability or degree of risk of the actual or candidate donor HCT to induce or to not induce graft vs. host disease (GVHD) in a HCT recipient.

In further particular aspects of the methods of the invention, one or more of the positive or negative gene expression profile of the candidate HCT donor, expression values of the positive or negative GVHD predictor genes of the candidate HCT donor, comparisions of the expression values to the respective predefined reference expression values for the positive or negative predictor genes of the candidate HCT donor, or comparisons of the linear or non linear combinations of expression values of the combination of positive and/or negative GVHD predictor genes, can be recorded or stored, for example, on an electronic medium, format or form, optionally that is computer readable or accessible.

In additional embodiments, methods of the invention can be performed using one or more probes or primers that specifically hybridizes to a gene, wherein the one or more probes or primers is selected from a probe or primer, or is derived from or based upon, a sequence listed in Tables 1 (RNA 1538, SEQ ID NOs:1-1546), 2, 2A (RNA143), 2B (RNA192), 3, 12 (HSK6), 13 (SG175), 15 (SG128) or 18 (SG64). For example, in a method of predicting and/or determining risk of a hematopoietic cell transplant (HCT) from a candidate donor to induce or to not induce GVHD in a HCT recipient, expression of one or more positive or negative GVHD predictor genes employs one or more probes or primers selected from, or derived from or based upon, a sequence in Tables 1 (RNA 1538, SEQ ID NOs:1-1546), 2, 2A (RNA143), 2B (RNA192), 3, 12 (HSK6), 13 (SG175), 15 (SG128) or 18 (SG64). Such probes and primers are presumed to hybridize to the respective genes listed in Tables 1 (RNA 1538, SEQ ID NOs:1-1546), 2, 2A (RNA143), 2B (RNA192), 3, 12 (HSK6), 13 (SG175), 15 (SG128) and 18 (SG64), and therefore, other such probes and primers based upon the nucleic acid sequence of the gene can be designed in order to measure or analyze expression of the gene as set forth herein. However, should the probes or primers hybridize to a different gene, methods of the invention can be performed using one or more of the particular probes (or probes of similar sequence and/or length) or primers listed in Tables 1 (RNA 1538, SEQ ID NOs:1-1546), 2, 2A (RNA143), 2B (RNA192), 3, 12 (HSK6), 13 (SG175), 15 (SG128) or 18 (SG64) as they are specific for a negative or positive GVHD predictor gene, no matter if the probe or primer does not hybridize to the particular gene listed in the Table.

Particular genes, the increased expression of which correlates with reduced risk of donor HCT inducing GVHD in a HCT recipient, are identified in Tables 1 (RNA 1538, SEQ ID NOs:1-1546), 2, 2A (RNA143), 2B (RNA192), 3, 12 (HSK6), 13 (SG175), 15 (SG128) or 18 (SG64), and are referred to as Negative Predictor genes. Negative Predictor genes according to the invention are therefore genes whose increased expression in CD4+ T cells or CD8+ T cells of candidate donors correlates with a reduced risk of inducing GVHD in a HCT recipient. Exemplary Negative Predictor genes are indicated in Tables 1 (RNA 1538, SEQ ID NOs:1-1546), 2, 2A (RNA143), 2B (RNA192), 3, and 13 (SG175), by an “N” symbol. In addition, for certain Negative Predictor genes, the greater the expression of the negative Predictor genes in donor CD4+ T cells or CD8+ T cells, the lower the risk or probability of donor HCT inducing GVHD in a HCT recipient.

As set forth herein, increased expression of negative predictor genes in CD4+ T cells correlates with HCT that does not induce GVHD and therefore indicates a reduced risk or probability of a donor HCT to induce GVHD in a HCT recipient. Accordingly, decreased expression of such negative predictor genes correlates and therefore indicates an increased risk or probability of a donor HCT to induce GVHD in a HCT recipient.

Particular genes, the increased expression of which correlates with increased risk of donor HCT inducing GVHD in a HCT recipient are identified in Tables 1 (RNA 1538, SEQ ID NOs:1-1546), 2, 2A (RNA143), 2B (RNA192), 3, 13 (SG175), 15 (SG128) or 18 (SG64), are referred to as Positive Predictor genes. Positive Predictor genes according to the invention are therefore genes whose increased expression in CD4+ T cells of candidate donors correlates with an increased risk of inducing GVHD in a HCT recipient. Exemplary Positive Predictor genes are indicated in Tables 1 (RNA 1538, SEQ ID NOs:1-1546), 2, 2A (RNA143), 2B (RNA192), 3, and 13 (SG175) by a “P” symbol. In addition, for certain positive predictor genes, the greater the expression of the Positive Predictor genes in donor CD4+ T cells, the greater the risk or probability of donor HCT inducing GVHD in a HCT recipient.

As set forth herein, increased expression of positive predictor genes in CD4+ T cells correlates with HCT that induces GVHD and therefore indicates an increased risk or probability of donor HCT to induce GVHD in a HCT recipient. Accordingly, decreased expression of such positive predictor genes correlates and therefore indicates a decreased risk or probability of a donor HCT to induce GVHD in a HCT recipient.

Negative and positive GVHD predictor genes according to the invention, and as listed in Tables 1 (RNA 1538, SEQ ID NOs:1-1546), 2, 2A (RNA143), 2B (RNA192), 3, 12 (HSK6), 13 (SG175), 15 (SG128) and 18 (SG64), can be measured or analyzed individually, or a plurality of such genes can be measured or analyzed in CD4+ T cells or CD8+ T cells of a candidate (or actual) HCT donor in order to predict or determine the risk of the candidate (or actual) donor HCT to induce or to not induce GVHD in a recipient, or any other methods of the invention. Thus, the grouping of Negative and Positive Predictor genes listed in the Tables is merely for purposes of illustration, and convenience, and is not in any way intended to mean that all genes within the Table must be analyzed, or that a minimum number of Negative and/or Positive Predictor genes in the Table must be analyzed, etc. Rather, in view of the guidance herein, any desired combination of Negative and/or Positive GVHD predictor genes in Tables 1 (RNA 1538, SEQ ID NOs:1-1546), 2, 2A (RNA143), 2B (RNA192), 3, 12 (HSK6), 13 (SG175), 15 (SG128) and 18 (SG64) can be measured or analyzed in order to perform the invention methods or used in producing the invention kits and arrays. Thus, by way of a non-limiting example, one or more negative and/or positive GVHD predictor genes selected from Table 2B (RNA192) can be combined with any gene listed in any of Tables 12 (HSK6), 13 (SG175), 15 (SG128) or 18 (SG64); one or more negative and/or positive GVHD predictor genes selected from Table 13 (SG175) can be combined with any gene listed in any of Tables 2B (RNA192), 12, (HSK6), 15 (SG128) or 18 (SG64); etc.

In accordance with the invention, the number of genes measured or analyzed can be a single gene (any sequence in Tables 1 (RNA 1538, SEQ ID NOs:1-1546), 2, 2A (RNA143), 2B (RNA192), 3, 12 (HSK6), 13 (SG175), 15 (SG128) or 18 (SG64), or any number of Negative and/or Positive GVHD predictor genes, up to all genes listed in Tables 1 (RNA 1538, SEQ ID NOs:1-1546), 2, 2A (RNA143), 2B (RNA192), 3, 12 (HSK6), 13 (SG175), 15 (SG128) or 18 (SG64), without limitation, and without inferring that any particular Negative or Positive Predictor genes must be analyzed. Likewise, analysis of gene ratios and combinations of positive and/or negative GVHD predictor genes can be undertaken based upon the sequences in Tables 1 (RNA 1538, SEQ ID NOs:1-1546), 2, 2A (RNA143), 2B (RNA192), 3, 12 (HSK6), 13 (SG175), 15 (SG128) or 18 (SG64). Again, the gene Tables set forth herein are intended to be representative and not limiting to particular genes or combinations of genes. For example, Table 3 is a representative 20 gene model (aka RNA20 model) in which analysis/measurement of such genes in CD4+ T cells of a donor provides a much greater ability to predict or determine risk of donor HCT inducing GVHD in a HCT recipient than by using the standard 10 out of 10 HLA matches between donor and recipient. Likewise, Tables 1 (RNA 1538, SEQ ID NOs:1-1546), 2, 2A (RNA143), 2B (RNA192), 3, 13 (SG175), 15 (SG128) and 18 (SG64) illustrate genes in which their analysis/measurement in CD4+ T cells of a candidate HCT donor provides a greater ability to predict or determine risk of donor HCT inducing GVHD in a HCT recipient than by using the standard 10 out of 10 HLA matches between donor and recipient. Other suitable models to predict or determine risk of donor HCT inducing GVHD in a HCT recipient can be readily constructed based upon any combination of the Negative and Positive Predictor genes in Tables 1 (RNA 1538, SEQ ID NOs:1-1546), 2, 2A (RNA143), 2B (RNA192), 3, 12 (HSK6), 13 (SG175), 15 (SG128) and 18 (SG64), and the teachings herein. Accordingly, the invention methods include measuring or analyzing one, or any combination of any number of the Negative and/or Positive Predictor genes, in Tables 1 (RNA 1538, SEQ ID NOs:1-1546), 2, 2A (RNA143), 2B (RNA192), 3, 12 (HSK6), 13 (SG175), 15 (SG128) or 18 (SG64). Likewise, invention compositions, such as kits, arrays and databases, include without limitation primers and/or probes for analysis or measurement of, or databases with expression profiles of, any one, or any combination of any number of the Negative and/or Positive GVHD Predictor genes in Tables 1 (RNA 1538, SEQ ID NOs:1-1546), 2, 2A (RNA143), 2B (RNA192), 3, 12 (HSK6), 13 (SG175), 15 (SG128) and 18 (SG64).

As used herein, a gene expression profile or “expression profile” refers to expression levels of one or more positive and/or negative GVHD predictor genes from CD4+ T cells or CD8+ T cells from a candidate HCT donor relevant to GVHD outcome prediction or determination. Such a profile can also include gene ratios, and combinations of expression values of positive and/or negative GVHD predictor genes. A profile corresponds to a particular candidate donor, and thus provides a way to score, identify or document suitability for their HCT as a donor for an HCT recipient.

Gene expression levels, profiles, scores, and other indicia of a candidate HCT donor or HCT recipient may be represented by any form of data which is suitable for use in the methods (e.g., comparisons and assessments) described herein. The levels, profiles, and scores may be presented as a physical representation (e.g., paper, such as a graph), computer (e.g., on a screen) or digital representation or as data stored in an electronic or computer-readable medium. Such data can be accessed by a user, for example, to identify a candidate donor HCT at low risk or probability of inducing GVHD in a HCT recipient.

As set forth herein, polymorphisms of negative and positive GVHD predictor genes listed in Tables 1 (RNA 1538, SEQ ID NOs:1-1546), 2, 2A (RNA143), 2B (RNA192), 3, 12 (HSK6), 13 (SG175), 15 (SG128) and 18 (SG64) are included. A polymorphism is a genetic variant at the RNA or genmonic DNA sequence level. Such polymorphisms are typically naturally occurring sequence variants, and can be single or multiple nucleotide changes. Polymorphisms may be silent in terms of not affecting the function, changing an amino acid residue of the encoded protein, or affecting activity, expression, half-life, etc. of the gene, mRNA or encoded protein. However, such polymorphisms may not be silent and may affect the function, change an amino acid residue of the encoded protein, or affect activity, expression, half-life, etc. of the gene, mRNA or encoded protein. Particular polymorphisms of negative and positive predictor genes listed in Tables 1-3 are known to one of skill in the art, and can be measured or analyzed as set forth herein or using other methods.

As used herein, the term “plurality” means 2 or more. As set forth herein, a plurality of positive and/or negative predictor genes can be measured or analyzed. Thus, 2 or more genes of Tables 1 (RNA 1538, SEQ ID NOs:1-1546), 2, 2A (RNA143), 2B (RNA192), 3, 12 (HSK6), 13 (SG175), 15 (SG128) or 18 (SG64) can be measured or analyzed in methods of the invention. In particular embodiments, the number of negative and/or positive predictor genes measured or analyzed is 2, 3, 4, 5, 6, 7, 8, 9, 10, 11, 12, 13, 14, 15, 16, 17, 18, 19, 20 or more, e.g., 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, etc. . . . up to all genes listed in Tables 1 (RNA 1538, SEQ ID NOs:1-1546), 2, 2A (RNA143), 2B (RNA192), 3, 12 (HSK6), 13 (SG175), 15 (SG128) and 18 (SG64).

Likewise, a plurality of analytes (e.g., primers, probes or antibodies) in the kits and/or arrays can bind to or hybridize with positive or negative predictor genes listed in Tables 1 (RNA 1538, SEQ ID NOs:1-1546), 2, 2A (RNA143), 2B (RNA192), 3, 12 (HSK6), 13 (SG175), 15 (SG128) or 18 (SG64), or expression products (proteins) encoded by such genes, to obtain expression values for the positive or negative GVHD predictor genes and comparing the expression value for the positive or negative predictor genes to a predefined reference expression value. Thus, analytes (e.g., primers, probes or antibodies) in the kits and/or arrays of the invention can include those that bind to or hybridize with 2, 3, 4, 5, 6, 7, 8, 9, 10, 11, 12, 13, 14, 15, 16, 17, 18, 19, 20 or more, e.g., 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, etc., up to all genes listed in Tables 1 (RNA 1538, SEQ ID NOs:1-1546), 2, 2A (RNA143), 2B (RNA192), 3, 12 (HSK6), 13 (SG175), 15 (SG128) and 18 (SG64), or expression products (proteins) encoded by such genes.

GVHD outcome prediction and/or determination, or classifying, categorizing, scoring or identifying according to risk or probability of a candidate donor HCT to induce or to not induce GVHD in a HCT recipient for a plurality of such genes is based upon the totality of comparisons of expression values of the plurality of positive or negative predictor genes to their respective predefined reference expression values. A gene expression profile or more simply an expression profile refers to expression of a plurality of Negative and/or Positive Predictor genes of a given candidate HCT donor, or is a dataset of expression values of the plurality of positive or negative predictor genes, or a dataset of linear or non linear combinations of expression values of the combination of positive and/or negative GVHD predictor gene comparisons, optionally compared to their respective predefined reference expression values, or 2 or more candidate HCT donors. Thus, a sufficient plurality of negative and/or positive predictor genes is measured for expression and an expression value, or combinations of expression values, is determined for each in order to provide a determination or prediction of risk of GVHD outcome, score, etc.

Of course, additional genes not listed in Tables 1 (RNA 1538, SEQ ID NOs:1-1546), 2, 2A (RNA143), 2B (RNA192), 3, 12 (HSK6), 13 (SG175), 15 (SG128) and 18 (SG64), and expression products (proteins) encoded by such genes, can be measured or analyzed, or included in methods of the invention, and analytes (e.g., primers, probes or antibodies) in the invention kits and arrays of the invention can bind to or hybridize with one or more genes not listed in Tables 1 (RNA 1538, SEQ ID NOs:1-1546), 2, 2A (RNA143), 2B (RNA192), 3, 12 (HSK6), 13 (SG175), 15 (SG128) or 18 (SG64). However, for purposes of predicting or determining degree of risk or probability of HCT from a candidate donor inducing or not inducing GVHD, the genes whose expression is measured or analyzed are one or more genes selected from among those genes listed in Tables 1 (RNA 1538, SEQ ID NOs:1-1546), 2, 2A (RNA143), 2B (RNA192), 3, 12 (HSK6), 13 (SG175), 15 (SG128) or 18 (SG64), or an expression product (protein) encoded by such genes.

In methods of the invention, GVHD outcome prediction or determination depends upon the expression level of one or more positive or negative predictor genes compared to a predefined or predetermined reference expression value for the particular positive or negative predictor gene. Expression of a gene from a candidate HCT donor closer to a value that correlates with higher risk of GVHD means that the particular gene is considered to indicate a higher risk of inducing GVHD, whereas expression of a gene from a candidate HCT donor closer to a value that correlates with a lower risk of GVHD means that the particular gene is considered to indicate a lower risk of inducing GVHD. In particular, for a positive predictor gene, a greater level of expression than the predefined or predetermined reference expression value for the particular positive predictor gene correlates with expression of the positive predictor gene in one or more HCT donors known to induce GVHD, and therefore indicates a higher degree of risk or probability of HCT inducing GVHD in a recipient. Accordingly, an expression value for the positive predictor gene greater than the predefined or predetermined reference expression value indicates that the HCT from the candidate donor is at higher risk of inducing graft vs. host disease (GVHD). For a negative predictor gene, greater level of expression than the predefined or predetermined reference expression value for the particular negative predictor gene correlates with expression of the negative predictor gene in one or more HCT donors known not to induce GVHD, and therefore indicates a lower degree of risk or probability of HCT inducing GVHD in a recipient. Accordingly, an expression value for the negative predictor gene greater than the predefined or predetermined reference expression value indicates that the HCT from the candidate donor is at lower risk of inducing graft vs. host disease (GVHD).

A predefined or predetermined reference expression value for positive and negative GVHD predictor genes is a value determined or set by expression analysis of donor HCT known to induce GVHD, at least to some extent in a HCT recipient, and donor HCT known not to induce GVHD in a HCT recipient. A predefined or predetermined reference expression value for positive and negative GVHD predictor genes (or analogously, linear or non linear combinations of expression values of the combination of positive and/or negative GVHD predictor genes has a predefined or predetermined reference value) is therefore a value set such that a greater level of expression is considered to indicate a higher or lower risk, respectively, of HCT of a candidate donor inducing GVHD in a HCT recipient. Of course, expression of a positive or negative GVHD predictor gene less than a predefined or predetermined reference expression value for the respective positive or negative predictor gene is considered to indicate a lower or higher risk, respectively, of HCT of a candidate donor to induce GVHD in a HCT recipient. A predefined or predetermined reference expression value is therefore considered a boundary value that separates (i.e., is a separatix) a higher and a lower risk or probability of GVHD outcome of a candidate donor HCT in a HCT recipient.

A predefined or predetermined reference expression value can be determined by discriminatory analysis. Such analysis determines the amount of positive or negative predictor gene expression that is statistically meaningful and that that separates GVHD outcome prediction or determination between a higher and a lower risk of inducing GVHD. For example, Discriminant Analysis, such as Linear Discriminant Analysis (LDA), or Quadratic Discriminant Analysis (QDA) provides a basis for discriminating gene expression values of candidate donor HCTs known to induce GVHD or known not to induce GVHD in a HCT recipient.

A predefined or predetermined reference expression value can be set by the user. For example, a predefined or predetermined reference expression value for a given positive or negative predictor gene can be set approximately or precisely midway between expression of the positive or negative predictor gene in CD4+ T cells or CD8+ T cells from an HCT donor known to induce GVHD and expression of the positive or negative predictor gene in CD4+ T cells or CD8+ T cells from an HCT donor known to not induce GVHD in a HCT recipient. Accordingly, an expression value for a positive predictor gene greater than the midway value indicates that the HCT from the candidate donor is at higher risk of inducing graft vs. host disease (GVHD); an expression value for a negative predictor gene greater than the midway value indicates that the HCT from the candidate donor is at lower risk of inducing graft vs. host disease (GVHD); an expression value for a positive predictor gene less than the midway value indicates that the HCT from the candidate donor is at lower risk of inducing graft vs. host disease (GVHD); and an expression value for a negative predictor gene less than the midway value indicates that the HCT from the candidate donor is at higher risk of inducing graft vs. host disease (GVHD).

Generally, a more reliable predefined or predetermined reference expression value can be based upon average or median expression of the positive or negative GVHD predictor gene in CD4+ T cells or CD8+ T cells from a plurality or multiple HCT donors that induce GVHD, and an average or median expression of the positive or negative predictor gene in CD4+ T cells or CD8+ T cells from a plurality or multiple HCT donors that do not induce GVHD in a HCT recipient. Accordingly, in one embodiment, a predefined or predetermined reference expression value for the positive predictor gene is set approximately or precisely midway between an average or median expression level of the positive predictor gene from two or more HCT donors that induce GVHD and two or more HCT donors that do not induce GVHD. In another embodiment, a predefined or predetermined reference expression value for the negative predictor gene is set approximately or precisely midway between an average or median expression level of the negative predictor genes from two or more HCT donors that induce GVHD and two or more HCT donors that do not induce GVHD. In more particular embodiments, the predefined reference expression value for the positive or negative predictor gene is set approximately or precisely midway between an average or median expression level of the positive or negative predictor genes from at least 2, 3, 4, 5 or more HCT donors (e.g., 6, 7, 8, 9, 10, 11, 12, 13, 14, 15, 16, 17, 18, 19, or more HCT donors, e.g., 20, 21, 22, 23, 24, 25, etc., or more) that induce GVHD and at least 2, 3, 4, 5 or more HCT donors (e.g., 6, 7, 8, 9, 10, 11, 12, 13, 14, 15, 16, 17, 18, 19, or more HCT donors, e.g., 20, 21, 22, 23, 24, 25, etc., or more) that do not induce GVHD.

A predefined or predetermined reference expression value for a positive or negative GVHD predictor gene can optionally be assigned a numerical value for ease of comparison of the expression value measured for the positive or negative predictor gene. Expression greater than the value can be taken to indicate a higher or lower risk of donor HCT inducing GVHD in a HCT recipient. In a particular embodiment, the predefined or predetermined reference expression value (e.g., midway value) is assigned a value of 0.5, and an expression value for one or more negative predictor genes greater than 0.5 indicates that the HCT from the candidate donor is at lower risk of inducing graft vs. host disease (GVHD). In another particular embodiment, the predefined or predetermined reference expression value (e.g., midway value) is assigned a value of 0.5, and an expression value for one or more positive predictor genes greater than 0.5 indicates that the HCT from the candidate donor is at higher risk of inducing graft vs. host disease (GVHD). Of course, should greater confidence in GVHD outcome prediction or determination be desired, the expression values required to be above the predefined or predetermined reference expression (numerical) value can be increased. Thus, for example, a negative predictor gene must have an expression value of 0.55 or greater (e.g., 0.60, 0.65, 0.70, 0.75, or 0.80) to indicate that the HCT from the candidate donor is at lower risk of inducing graft vs. host disease (GVHD). In another example, a positive predictor gene must have an expression value of 0.55 or greater (e.g., 0.60, 0.65, 0.70, 0.75, or 0.80) to indicate that the HCT from the candidate donor is at higher risk of inducing graft vs. host disease (GVHD).

The reference expression value (or predefined reference value) can be set to a higher or lower threshold. Such reference expression values therefore can be adjusted to increase reliability, accuracy, reproducibility, and to account for variables such as statistical error, etc., in order to improve the robustness of GVHD determination/prediction. Generally, to reduce or minimize the risk or probability of candidate donor HCT inducing GVHD in a HCT recipient (i.e., to reduce false negatives, i.e., to correctly predict a candidate donor who is at increased risk of inducing GVHD in a recipient), the user can select for higher expression of negative predictor genes by setting the reference expression value higher, and/or lower expression of positive predictor genes by setting the reference expression value lower, in a gene expression profile of CD4+ T cells or CD8+ T cells from a candidate HCT donor.

An expression value obtained for the positive or negative GVHD predictor genes can be adjusted or normalized relative to expression of one or more reference genes prior to comparing the expression value of the positive or negative predictor gene to the predefined reference expression value for the positive or negative predictor gene. Methods for normalizing the level of gene expression are known to those of skill in the art. For example, expression of a positive or negative predictor gene can be normalized on the basis of the relative ratio of the mRNA level of the gene to the mRNA level of a reference gene, such as a gene whose expression is constitutive and at a relatively constant level in CD4+ T cells or CD8+ T cells, or a positive or negative predictor gene whose expression is not used to determine the expression value, so that variations in sample amount, extraction efficiency, extracted amount, or measurement chemistry or instrumentation performance are reduced in measuring gene expression amounts or level. In particular embodiments, a reference gene is a housekeeping gene (e.g., in Tables 12 or 13).

As used herein, “housekeeping gene” is a gene the expression of which is substantially the same from sample to sample or from tissue to tissue, or one that is relatively refractory to change in response to external stimuli. A housekeeping gene can be any gene other than the positive or negative predictive gene of interest for which the expression value is determined that will allow normalization of sample RNA or any other marker that can be used to normalize for the amount of total RNA added to each reaction. Non-limiting examples include those designated with the “HSK” symbol in Tables 1 (RNA 1538), 2A, 2B (RNA 192), 12 and 13, and/or more particularly, eukaryotic translation initiation factor 4H (EIF4H) transcript variant 1, 3 beta actin (ACTB), aldolase A (ALDOA), lactate dehydrogenase A (LDHA), phosphoglycerate kinase 1 (PGK1), transferrin receptor (TFRC), tubulin beta (TUBB), tubulin beta 2A (TUBB2A), thioredoxin (TXN), ubiquitin C (UBC), or ubiquitin-activating enzyme E1 (UBE1).

The term, “combination,” when used in reference to one or more GVHD predictor genes, refers to a minimal combination of 2 predictor genes, and could also be combinations of more predictor genes, such as 3, 4, 5, 6, 7, 8, 9, 10, 11, 12, 13, 14, 15, 16, 17, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 57, 48, 49, 50, or more, up to “n” positive and/or negative GVHD predictor genes, where “n” is a natural number. Thus, by way of illustration only and without limitation, a combination could be 2 or more positive GVHD predictor genes in combination, 2 or more negative GVHD predictor genes in combination, or 2 or more positive and/or negative GVHD predictor genes in combination, the number of such combinations of positive and/or negative predictor genes being 2² for 2 genes in combination (P-P, P-N, N-P, N-N), 2³ for 3 genes in combination (P-P-P, P-P-N, P-N-P, P-N-N, N-P-P, N-P-N, N-N-P, N-N-N), 2⁴, 2⁵, or 2ⁿ for any higher order combination of “n” genes. In the context of combinations, a “predefined reference value,” for example, as used in the comparison step in accordance with the methods of the invention, also refers to a combination of expression values, and not a single expression value.

A “linear combination,” when used in reference to “combinations of” expression values, refers minimally to the difference of 2 expression values, X−Y, or the difference of the logarithm of 2 expression values, log X−log Y, or the sum of 2 expression values, X+Y, or the sum of the logarithm of 2 expression values, log X+log Y, or also combined differences and/or sums of more than 2 expression values, for which the expression value or the logarithm of the expression value of any of the genes may be multiplied by a factor, “c,” where “c” is a real number, and where the value of “c” may differ for each of the genes, and for which a constant term, “d,” can be added or subtracted to the expression value of any of the genes, where “d” is a real number, and where the value of “d” may differ for each of the genes. A “non-linear combination,” when used in reference to expression values, refers minimally to the ratio of 2 expression values, X/Y, or the ratio of the logarithm of 2 expression values, log X/log Y, or the product of 2 expression values, X*Y, or the product of the logarithm of 2 expression values, log X*log Y, or also combined ratios and/or products of more than 2 expression values, for which the expression value or the logarithm of the expression value of any of the genes may be exponentiated by an exponent, “b,” where “b” is a real number, and where the value of “b” may differ for each of the genes, and for which the expression value or the logarithm of the expression value of any of the genes may be multiplied by a factor, “c,” where “c” is a real number, and where the value of “c” may differ for each of the genes, and for which a constant term, “d,” can be added or subtracted to the expression value or the logarithm of the expression value of any of the genes, where “d” is a real number, and where the value of “d” may differ for each of the genes.

Normalization of gene expression may be performed in a straightforward manner for predictive models that involve pairs of predictor genes in competitive relationships, i.e. a ratio of gene 1 over gene 2 in a predictor gene pair (referred to herein as a ratiometric gene pair, or RGP), obviating the need for an additional reference gene (see Examples). Instead of reporting the level of a positive or negative predictor gene with respect to a separate housekeeping gene and/or reference sample, the level of predictor gene 1 with respect to predictor gene 2 (their ratio) provides a relative expression measurement ratio with high information content.

Accordingly, an expression value for positive or negative GVHD predictor genes can also be represented as a ratio, as in a ratiometric gene pair (RGP). Ratios of gene expression data can be represented in a variety of ways. In one embodiment, an expression value is represented by a ratio of gene expression, denoted a ratiometric gene pair (RGP), of the positive or negative GVHD predictor gene to one or more reference genes. In a more particular embodiment, an expression value is represented by a ratio of gene expression, denoted a ratiometric gene pair (RGP), of the positive or negative predictor GVHD gene to a reference gene, and is represented by the formula “N/D,” (numerator/denominator), where the numerator value “N” is the expression level of the positive or negative GVHD predictor gene and the denominator value “D” is the expression level of one or more reference genes. The N and D values can optionally reflect an average or median expression of one or more positive or negative GVHD predictor genes, or one or more reference genes, respectively, and optionally reflect expression in a plurality of samples. Such RGPs include combinations of positive and negative GVHD predictor genes (N-P and P-N), combinations of positive GVHD predictor genes (P-P), and combinations of negative GVHD predictor genes (N-N).

For such expression value determination, expression normalization and expression ratio determinations (e.g., RGPs), a reference gene can be a housekeeping (HSK) gene, or a positive or negative GVHD predictor gene that is different from the positive or negative predictor gene used to obtain the ratio of gene expression, or any other gene selected by the user.

In accordance with the invention, positive and negative GVHD predictor genes in which expression is measured for GVHD, whether expression of a single gene or using ratios of two (or more) genes (RGPs, pairs of gene pairs, etc.), or combinations of genes, are listed in and can be selected from Tables 1 (RNA 1538), 2A (RNA 143), 2B (RNA 192), 3, 13 (SG175), 15 (SG128), or 18 (SG64), or a polymorphism thereof. In one embodiment, at least one of the positive or negative GVHD predictor genes whose expression is measured is selected from one or more single genes (SGs) set forth in Tables 1 (RNA 1538), 2A (RNA143), 2B (RNA 192), 3, 13 (SG175), 15 (SG128) or 18 (SG64), or is selected from ratiometric gene pairs (RGPs) or single genes (SGs) set forth in Tables 1 (RNA 1538), 2A (RNA143), 2B (RNA 192), 3, 13 (SG175), 15 (SG128), or 18 (SG64). Exemplary non-limiting ratiometric gene pairs (RGPs) are set forth in and can be selected from Tables 14 (RGP348) and 17 (VmodRGP100), and exemplary non-limiting examples of multiple genes in ratios such as “pairs of gene pairs,” are set forth in and can be selected from Table 16 (“PRGP348”). Accordingly, expression of single genes, ratios of genes (e.g., RGPs) and combinations of genes, including multi-gene ratios of negative, positive and/or mixtures of negative and positive GVHD predictor genes from any of Tables 1 (RNA 1538), 2A (RNA143), 2B (RNA 192), 3, 13 (SG175), 15 (SG128), 17 (VmodRGP100) and 18 (VmodSG64), in any combination, can be undertaken to perform the invention.

In a more particular embodiment of the invention, the negative and/or positive GVHD predictor genes used to predict or determine risk that a hematopoietic cell transplant (HCT) from a candidate donor will induce or not induce graft vs. host disease (GVHD) in a HCT recipient is selected from one or more genes set forth in Table 18 (VmodSG64). In another more particular embodiment of the invention, the negative and/or positive GVHD predictor genes used to predict or determine risk that a hematopoietic cell transplant (HCT) from a candidate donor will induce or not induce graft vs. host disease (GVHD) in a HCT recipient is a plurality of ratiometric gene pairs (RGPs) of two or more genes selected from the genes listed in Tables 1 (RNA 1538), 2, 2A (RNA 143), 2B (RNA 192), 3, 13 (SG175), 15 (SG128) or 18 (SG64). In a further particular embodiment of the invention, the ratiometric gene pairs (RGPs) used to predict or determine risk that a hematopoietic cell transplant (HCT) from a candidate donor will induce or not induce graft vs. host disease (GVHD) in a HCT recipient is one or more gene pairs (RGPs) selected from the genes listed in Table 17 (VmodRGP100). In an additional particular embodiment of the invention, the negative and/or positive GVHD predictor genes include a combination of single genes (SGs) and ratiometric gene pairs (RGPs) to predict or determine risk that a hematopoietic cell transplant (HCT) from a candidate donor will induce or not induce graft vs. host disease (GVHD) in a HCT recipient is a plurality of genes selected from the single genes (SGs) listed in Table 18 (VmodSG64) and ratiometric gene pairs (RGPs) selected from the RGPs listed in Table 17 (VmodRGP100).

In accordance with the invention, where a plurality of positive and/or negative GVHD predictor genes are measured or analyzed for expression, typically there will be a threshold (e.g., minimum) number of genes, or expression levels or amounts or types of genes evaluated, in order to predict or determine that the candidate donor HCT is at high risk or at low risk to induce graft vs. host disease (GVHD) in a HCT recipient. Evaluation refers to analysis based upon one or more criteria including, but not limited to, gene expression greater or less than a threshold expression level, or the number of positive and/or negative GVHD predictor genes above or below a threshold, which can be set by the user, or the GVHD predictive direction of particular genes whose expression tends to have a high correlation with GVHD outcome. All of such criteria, which can be set by the user, can be based upon the desired degree of confidence or accuracy. By way of a non-limiting example, the number of single genes (SGs), gene expression ratios (e.g., RGPs) or multi-gene ratios (e.g., PRGPs, such as Table 16) measured or analyzed for expression is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 57, 48, 49, 50, or more genes and/or gene expression ratios.

The number of genes or expression levels, or linear or non-linear combination of expression values, could be represented by a percent of the total number of genes whose expression is measured, for example, at least 30%, 40%, 50%, 60%, 70%, 80% or more of the total number of positive and/or negative predictor genes. Thus, if expression of a total of 10 predictor genes are measured, a threshold could be 3, 4, 5, 6, 7, 8 or more of the genes must indicate a low or high risk of HCT inducing GVHD in order to predict or determine that the HCT is at low or high risk of inducing GVHD. In particular embodiments, a majority of the positive or negative GVHD predictor genes must indicate a high risk of inducing graft vs. host disease (GVHD) in a HCT recipient to predict or determine that the candidate donor HCT is at high risk to induce graft vs. host disease (GVHD) in a HCT recipient; or a majority of the positive or negative GVHD predictor genes must indicate a low risk of inducing graft vs. host disease (GVHD) in a HCT recipient to predict or determine that the candidate donor HCT is at low risk to induce graft vs. host disease (GVHD) in a HCT recipient. In particular embodiments, when the number of positive or negative GVHD predictor genes, or the combination of positive and/or negative GVHD predictor genes, indicating that the HCT from the candidate donor is at higher risk of inducing GVHD is greater than the number of positive or negative predictor genes, or the combination of positive and/or negative GVHD predictor genes indicating that the HCT from the candidate donor is at lower risk of inducing GVHD in a HCT recipient, this predicts or determines a higher risk of the HCT of a candidate donor to induce GVHD in an HCT recipient. In more particular embodiments, when the number of positive or negative GVHD predictor genes, or the combination of positive and/or negative GVHD predictor genes, indicating that the HCT from the candidate donor is at lower risk of inducing GVHD is greater than the number of positive or negative predictor genes, or the combination of positive and/or negative GVHD predictor genes, indicating that the HCT from the candidate donor is at higher risk of inducing GVHD in a HCT recipient, predicts or determines a lower risk of the HCT from a candidate donor to induce GVHD in an HCT recipient.

In further particular embodiments, at least 66% of the positive or negative predictor genes must indicate a high risk of inducing graft vs. host disease (GVHD) in a HCT recipient to predict or determine that the candidate donor HCT is at high risk to induce graft vs. host disease (GVHD) in a HCT recipient; or at least 66% of the positive or negative predictor genes must indicate a low risk of inducing graft vs. host disease (GVHD) in a HCT recipient to predict or determine that the candidate donor HCT is at low risk to induce graft vs. host disease (GVHD) in a HCT recipient. In an additional particular embodiment, at least 75% of the positive or negative predictor genes must indicate a low risk of inducing graft vs. host disease (GVHD) in a HCT recipient to predict or determine that the candidate donor HCT is at low risk to induce graft vs. host disease (GVHD) in a HCT recipient.

By way of illustration only, one non-limiting model for ascertaining the risk of GVHD in a recipient is to assign each positive and/or negative GVHD predictor gene whose expression is analyzed a “vote” for purposes of ascertaining risk of inducing or not inducing GVHD. The votes are tabulated depending upon whether the expression values, or combinations of expression values, obtained from each gene measured indicates an increased or reduced risk of GVHD. For example, if expression of a total of 10 positive and/or negative predictor genes is measured, a majority (i.e., 6 of the 10) might indicate a reduced risk, and 4 out of 10 might indicate an increased risk of GVHD. Thus, 6 genes would vote reduced risk of GVHD, and 4 genes would vote increased risk of GVHD. Depending upon the genes and their ability to accurately predict risk of GVHD or not, a majority of votes for such a 10 gene voting model may be sufficient to conclude a reduced risk of inducing GVHD. If greater confidence in predictive accuracy is desired, the threshold number of gene “votes” required to predict a particular GVHD outcome, can be increased, for example, from 6 to 7 out of 10 genes, or from 6 to 8 out of 10 genes, or greater.

In accordance with the invention, one exemplary model is to assign a “vote” to each gene whose expression is measured, and depending upon the expression value obtained from each gene assign a vote, and based upon the sum total of votes, risk of inducing or not inducing GVHD is determined or predicted. In one embodiment, a plurality of expression values for negative or positive GVHD predictor genes is determined, and a vote is assigned to each negative or positive predictor gene according to whether the expression value for the gene indicates the risk of the candidate or actual donor to induce or not to induce GVHD. Subsequently, a score is assigned to the candidate or actual donor based upon the total number of votes indicative or not indicative of inducing or not inducing GVHD in a HCT recipient. In particular aspects, if more than 50% of the votes are indicative of inducing GVHD, then the score reflects an increased risk of the hematopoietic cell transplant (HCT) from the candidate or actual donor to induce GVHD in a HCT recipient; or if more than 50% of the votes are indicative of not inducing GVHD, then the score reflects a decreased risk of the hematopoietic cell transplant (HCT) from the candidate or actual donor to induce GVHD in a HCT recipient. In additional aspects, when at least 55%, 60%, 65%, 70%, 75%, 80%, 85%, 90%, 95%, or more of the votes are indicative of inducing GVHD, then the score reflects a increased risk of the hematopoietic cell transplant (HCT) from the candidate or actual donor to induce GVHD in a HCT recipient; or wherein when at least 55%, 60%, 65%, 70%, 75%, 80%, 85%, 90%, 95%, or more of the votes are indicative of not inducing GVHD, then the score reflects a decreased risk of the hematopoietic cell transplant (HCT) from the candidate or actual donor to induce GVHD in a HCT recipient.

Numerous non-limiting, representative voting models (Vmods) that predict or determine risk of inducing and not inducing GVHD, are disclosed herein. Such non-limiting examples of voting models include the combination of single genes (SGs) and ratiometric gene pairs (RGPs) set forth in: SG43RGP46-GPperformance; SG42RGP21-GPminimalist; SG43RGP37-GPconnectivity; SG43RGP51-PRGPminranksort; SG43RGP55-PRGPmedranksort; SG43RGP36-RGPgreedysearch; or SG21RGP28-RGPmaxgreedysearch, each of which combinations include the SGs and RGPs. The SGs and RGPs that are comprised in each of the voting models (Vmods) and whose expression is measured is indicated by an “x” in Tables 17 and 18.

Methods of the invention are typically superior to identifying GVHD negative donor HCT based upon having 10 out of 10 HLA marker loci matches of the HCT donor to a HCT recipient. In particular embodiments, a method predicts a donor HCT that induces or does not induce GVHD in a HCT recipient with an accuracy of at least 60%, at least 70%, at least 80%, or at least 90%. In another particular embodiment, the accuracy of predicting a GVHD negative donor is the probability or degree of risk of correctly identifying a GVHD negative donor within a group of candidate HCT donors classified as negative by 10 out of 10 HLA marker loci matches with an HCT recipient.

As used herein, the term “measuring” or “analyzing” in the context of determining expression or quantifying amounts of gene expression can refer to absolute or to relative quantification. In the context of gene expression, measuring refers to a laboratory procedure involving one or more isolating, purifying, processing, manipulating, extracting, or determining steps practiced with a sample or specimen, such as CD4+ T cells or CD8+ T cells, the amount of expression of one or more genes, which is distinct from any mental steps. Absolute quantification may be accomplished by inclusion of a known concentration(s) of one or more target nucleic acids or expression products and referencing the hybridization or binding intensity of unknowns to the known target nucleic acids or expression products (e.g., through generation of a standard curve). Alternatively, relative quantification can be accomplished by comparing signals between two or more genes, or between two or more samples to quantify the changes in signal and, by implication, transcript or expression product and therefore gene expression amounts.

Comparing can be carried out by visual inspection, or by using a computer algorithm. Examples of algorithms include linear or nonlinear regression algorithms; linear or nonlinear classification algorithms; ANOVA (analysis of variance); computational neural network algorithms; computational genetic algorithms; support vector machines algorithms; hierarchical analysis or clustering algorithms; hierarchical algorithms using decision trees; kernel based machine algorithms; table look-up algorithms; discriminatory algorithms such as partial least squares algorithms, matching pursuit algorithms, Fisher discriminate analysis algorithms, principal components analysis algorithms, singular value decomposition algorithms; Bayesian probability function algorithms; Markov Blanket algorithms; hidden Markov algorithms; deterministic optimization algorithms; stochastic search optimization or simulated annealing algorithms; recursive feature elimination or entropy-based recursive feature elimination algorithms; algorithms arranged in combination; plurality of algorithms arranged in a committee network; and forward floating search or backward floating search algorithms. Further methods to obtain values for determining or predictive GVHD outcome using one or more single genes or ratiometric gene pairs (RGPs, pairs of gene pairs (PRGPs), etc., as set forth herein are described in Example 20.

Candidate and actual HCT donors and HCT recipients include animals, typically mammalian animals (mammals), such as humans. Humans include, but are not limited to, family members genetically related to a candidate HCT recipient. Humans also include non-family members which are non-genetically related to a candidate HCT recipient, including non-familial actual or candidate HCT donors having HLA matches with a candidate HCT recipient. More specifically, an actual or a candidate HCT donor and a HCT recipient have 10 out of 10 or 9 out of 10 human leukocyte antigen (HLA) marker loci matches, for example, HLA marker loci matches of: HLA-A, HLA-B, HLA-C, HLA-DRB1 and HLA-DQB1 loci, or any combination of 4 of HLA-A, HLA-B, HLA-C, HLA-DRB1 or HLA-DQB1 loci matches. Such HLA marker loci matches may have been determined either serologically or by sequence analysis of HLA genes. Animals appropriate for analysis include those that may be a HCT donor for an HCT recipient of another animal, for example, an animal model of HCT GVHD.

For purposes of defining an actual or a candidate donor HCT that induces GVHD, as set forth herein if an HCT recipient manifests symptoms of GVHD following transplantation from the donor, the donor HCT is considered to induce GVHD. For purposes of defining an actual or a candidate donor HCT that does not induce GVHD, as set forth herein if an HCT recipient does not manifest symptoms of GVHD following transplantation from the donor, the donor HCT is considered to not induce GVHD. Occassionally, a candidate or actual donor HCT may be defined as a donor HCT that does not induce GVHD, for cases in which the recipient manifests only the least serious form of acute GVHD, i.e., acute grade I GVHD, and no other forms of acute or chronic GVHD at any time after HCT, following transplantation from the donor.

GVHD can be classified or grouped according to symptom severity and duration, and is classified herein to be within Groups 1-6, which generally reflect differences in severity. Exemplary classes begin with Group 1, which exhibits neither acute nor chronic GVHD, and ends with Group 6, showing severe acute grade 3 or 4 GVHD and extensive chronic GVHD. Group 5 also shows grade 3 or 4 GVHD, but no chronic GVHD. Group 4 and Group 3 show grade 1 or 2 acute GVHD, with and without chronic GVHD, respectively. Group 2 shows only chronic GVHD and no acute GVHD. Acute grade 3 or 4 GVHD characterize the most intense and life-threatening form of GVHD, while acute grade 1 or 2 GVHD is much less severe and occasionally may be considered mild. The grade classifications of acute GVHD are multi-symptom diagnostic gradations of well-established in medical practice for physician grading of GVHD severity. Although the definitions of the Groups are per se, they are medically meaningful GVHD-severity groups. Other classifications are possible. For example, the terms acute GVHD, chronic GVHD, grades 0-4 are established, accepted, medically defined terms; whereas Groups 1-6 are terms defined herein.

Methods of the invention further include assigning an actual or a candidate HCT donor a score, or identifying an actual or a candidate HCT donor. Such a score or identification can be based upon the HCT donor gene expression profile, expression value(s) for the positive and/or negative predictor gene(s) of the HCT donor, or the totality of information for a candidate HCT donor, such as also including the HLA marker loci profile. The score or identification can reflect the probability or degree of risk of the actual or candidate donor HCT to induce or to not induce graft vs. host disease (GVHD) in a HCT recipient, based upon risk prediction or determination. The score or identification can also reflect a class or group of GVHD predicted or determined to occur, which can indicate GVHD outcome or severity (e.g., as defined by Groups 1-6 as set forth herein, or as defined by acute grades I, II, III or IV GVHD, with or without chronic GVHD, or chronic GVHD without acute GVHD).

As set forth herein, the invention is exemplified by analysis of expression levels of genes, including negative and/or positive GVHD predictor genes, as well as reference genes (e.g., HSK genes), in CD4+ T cells. Methods of the invention can also employ other types of T cells. For example, methods of the invention can ascertain expression levels of negative and/or positive GVHD predictor genes, as well as reference genes (e.g., HSK genes), in CD8+ T cells. Accordingly, the invention can be practiced with various T cells, including but not limited to, CD4+ T cells, CD8+ T cells, T-regulatory cells, and mixtures of these and other T cell sub-types.

Biological samples include any sample capable of having a biological material. Biological samples include any biological material that includes cellular material from a candidate HCT donor. Typically, such samples include immunological cells, for example, CD4+ T cells and/or CD8+ T cells. Biological samples therefore include a biological material or fluid or any material that includes nucleic acid, such as DNA, RNA or polypeptide (protein) suitable for measurement or analysis of expression of one or more positive and/or negative predictor genes from a candidate HCT donor, for GVHD outcome prediction or determination. A biological sample therefore need only be suitable for measuring or analyzing expression of one or more positive and/or negative predictor genes, and that includes nucleic acid and/or protein that correlates with a GVHD outcome. Typically, biological samples include CD4+ T cells, CD8+ T cells or cellular material. Non-limiting examples include blood, blood cells (e.g., peripheral blood mononuclear cells), serum, plasma, bone marrow, mucus, saliva, feces, cerebrospinal fluid, or urine.

A biological sample can be transformed, processed or manipulated, for example, to determine the presence of, or measure or analyze gene expression or expression product amounts or levels or function. Typically, a biological sample is transformed or processed to purify or isolate a nucleic acid (e.g., total, or mRNA) or a gene expression product (e.g., a protein or fragment) that directly or indirectly indicates expression and/or amounts or levels of one or more positive and/or negative GVHD predictor genes. Thus, samples also include nucleic acid and protein purified, isolated, derived from, extracted from, or obtained from CD4+ T cells or CD8+ T cells from a candidate HCT donor.

Negative and/or positive GVHD predictor gene expression levels may be determined by measuring mRNA (or a cDNA reverse transcribed from the mRNA) from a sample comprising CD4+ T cells or CD8+ T cells from a candidate HCT donor. A negative or positive GVHD predictor gene may be capable of encoding a protein. Accordingly, gene expression levels may be determined by measuring an expression product, such as a polypeptide or protein. Expression of transcripts and/or proteins encoded by negative and/or positive predictor genes set forth in Tables 1 (RNA 1538, SEQ ID NOs:1-1546), 2, 2A (RNA143), 2B (RNA192), 3, 13 (SG175), 15 (SG128), or 18 (SG64) may be measured and/or analyzed by any of a variety of methods known to one of skill in the art.

Suitable nucleic acid samples for detection, measuring or analysis include transcripts of interest (i.e., transcripts, such as RNA, preprocessed RNA, or mRNA derived from positive and/or negative predictor genes of HCT inducing GVHD in a HCT recipient). Thus, when measuring or analyzing RNA expression (e.g., mRNA), such RNA can be measured directly.

Suitable nucleic acid samples for screening also include nucleic acids derived from a transcript of interest (e.g., such as cDNA from the mRNA derived from positive and/or negative predictor gene). A nucleic acid derived from a transcript refers to a nucleic acid for whose synthesis an mRNA transcript or a subsequence thereof (ultimately) served as a template. Examples of such nucleic acids include cDNA reverse transcribed from a transcript, an RNA transcribed from that cDNA, a DNA amplified from the cDNA, an RNA transcribed from the amplified DNA, etc.—all derived from the transcript, and measurement of such derived products is indicative of the presence and/or amount of positive and/or negative gene expression. For example, RNA of a positive or negative predictor gene can be reverse transcribed into cDNA (complementary DNA), which can then be measured, since the amount of cDNA correlates with the amount of RNA expressed.

In general, nucleic acid (e.g., DNA or RNA) in a sample can be detected by any suitable method or technique of measuring or detecting a gene sequence or expression or amount. Non-limiting exemplary methods of measuring gene (e.g., nucleic acid expression) include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other nucleic acid hybridization platform. For measuring RNA expression, methods include, but are not limited to: extraction of cellular mRNA and Northern blotting using labeled probes that hybridize to transcripts of all or part of one or more of the negative and/or positive predictor genes set forth herein; amplification of mRNA expressed from one or more of the negative and/or positive predictor genes using specific primers, polymerase chain reaction (PCR), quantitative PCR (q-PCR), and reverse transcriptase-polymerase chain reaction (RT-PCR), followed by quantitative detection of the product; and extraction of total RNA from cells, which is then processed (e.g. reverse transcribed or amplified), labeled and used to probe cDNAs or oligonucleotides encoding all or part of the negative and/or positive predictor genes; and in situ hybridization. Primers for RT-PCR corresponding to the positive and negative GVHD predictor genes, and the housekeeping genes, are listed, for example, in Table 2B (RNA 192), and are specified according to commercially available ABI Assay ID numbers. Other primers and probes can be derived from or based upon gene sequences listed in Tables 1 (RNA 1538, SEQ ID NOs:1-1546), 2, 2A (RNA143), 2B (RNA192), 3, 13 (SG175), 15 (SG128), and 18 (SG64).

Methods of isolating RNA, such as total or mRNA, are known to those of skill in the art. Non-limiting examples include, for example, acid guanidinium-phenol-chloroform extraction to obtain total nucleic acid from a biological sample, and isolating mRNA by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ad. Greene Publishing and Wiley-Interscience, New York (1987)).

In embodiments in which nucleic acid is amplified, whatever amplification method is used, if a result that reflects gene expression amounts or levels is desired, a method is used that maintains or controls for the relative frequencies of the amplified nucleic acids to achieve quantitative amplification. Various methods of “quantitative” amplification are known to those skilled in the art. For example, quantitative PCR involves simultaneously co-amplifying a known quantity of a control sequence using the same primers. This provides an internal standard that may be used to calibrate the PCR reaction. Thus, primers and/or probes specific to the internal standard can be used for quantification of the amplified nucleic acid. Other suitable amplification methods include, but are not limited to polymerase chain reaction (PCR; Innis, et al., PCR Protocols. A Guide to Methods and Application. Academic Press, Inc. San Diego, (1990)), ligase chain reaction (LCR; Wu and Wallace, Genomics, 4:560; Landegren et al., Science, 241: 1077; and Barringer, et al., Gene, 89:117)), transcription amplification (Kwoh et al., Proc. Natl. Acad. Sci. USA, 86:1173), and self-sustained sequence replication (Guatelli et al., Proc. Nat. Acad. Sci. USA, 87:1874).

Accordingly, gene expression levels may in general be measured or analyzed by detecting RNA, such as mRNA from cells (or cDNA thereof) and/or detecting gene expression products, such as a polypeptide or protein. Expression of the transcripts and/or proteins encoded by the positive and/or negative predictor genes described herein Tables 1 (RNA 1538, SEQ ID NOs:1-1546), 2, 2A (RNA143), 2B (RNA192), 3, 13 (SG175), 15 (SG128), and 18 (SG64) may be measured by any of a variety of known methods in the art. Analytes according to the invention therefore include nucleic acid sequences.

As used herein, the terms “nucleic acid” and “polynucleotide” and the like refer to at least two or more ribo- or deoxy-ribonucleic acid bases (nucleotides) that are linked through a phosphoester bond or equivalent covalent bond. Nucleic acids include polynucleotides and polynucleosides. Nucleic acids include single, double or triplex stranded, circular or linear, molecules. Nucleic acids include sense and anti-sense sequences, for example, sense and anti-sense sequences that bind to all or a portion of any sequence in Tables 1 (RNA 1538), 2, 2A, 2B (RNA 192) and/or 3, or a complementary sequence thereof of any sequence in Tables 1 (RNA 1538), 2, 2A, 2B (RNA 192) and/or 3. Exemplary nucleic acids include but are not limited to: total RNA, mRNA, DNA, cDNA, genomic nucleic acid, naturally occurring and non naturally occurring nucleic acid, e.g., synthetic nucleic acid.

Nucleic acids can be of various lengths. Nucleic acid lengths typically range from about 10 nucleotides to 20 Kb, or any numerical value or range within or encompassing such lengths, e.g., 10 nucleotides to 250 Kb, 1 to 15 Kb or less, 1000 to about 5000 nucleotides or less, 500-1000 nucleotides in length. Nucleic acids can also be shorter, for example, 100 to about 500 nucleotides, or from about 10 to 25, 25 to 50, 50 to 100, 100 to 250, or about 250 to 500 nucleotides in length, or any numerical value or range or value within or encompassing such lengths. In particular aspects, a nucleic acid sequence has a length from about 5-10, 10-20, 20-30, 30-40, 40-50, 50-60, 60-70, 70-80, 80-90, 90-100, 100-150, 150-200, 200-250, 250-300, 300-400, 400-500, 500-1000, 1000-2000, nucleotides, or any numerical value or range within or encompassing such lengths. Shorter polynucleotides are commonly referred to as “oligonucleotides” or “probes” or “primers” of single- or double-stranded DNA, typically a length from about 10-20, 20-30, 30-50, 50-100 nucleotides. However, there is no upper limit to the length of such oligonucleotides.

Nucleic acids include, for example, polynucleotides and oligonucleotides (primers and probes) that hybridize to a negative and/or positive predictor gene sequence (or a transcript, RNA or cDNA thereof), for example, to all or a portion of any sequence in Tables 1 (RNA 1538, SEQ ID NOs:1-1546), 2, 2A (RNA143), 2B (RNA192), 3, 13 (SG175), 15 (SG128), or 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), and 18 (SG64). Such hybridizing nucleic acids allow detection of a target sequence, transcript, or a complementary or amplified sequence, and can be used in the methods of the invention for predicting or determining the risk of HCT to induce or to not induce GVHD in a HCT recipient, as well as in the kits and arrays of the invention.

In order to detect or measure expression of a negative and/or positive predictor gene, a nucleic acid (e.g., oligo- or poly nucleotide probe or primer) can “hybridize” to all or a portion of the corresponding negative and/or positive predictor gene sequence (or an RNA transcript or cDNA thereof) or complementary sequence, i.e., to all or a portion of any sequence in Tables 1 (RNA 1538, SEQ ID NOs:1-1546), 2, 2A (RNA143), 2B (RNA192), 3, 13 (SG175), 15 (SG128), or 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), or 18 (SG64), which refers to the binding between two or more nucleic acid sequences. Sequences “sufficiently complementary” allow stable hybridization of a nucleic acid sequence to a target sequence (a negative and/or positive predictor gene sequence, or a transcript, RNA or cDNA thereof, for example, all or a portion of any sequence in Tables 1 (RNA 1538, SEQ ID NOs:1-1546), 2, 2A (RNA143), 2B (RNA192), 3, 13 (SG175), 15 (SG128), and 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), and 18 (SG64), and therefore detection even if the two sequences are not completely complementary. Detection may either be direct (i.e., resulting from a probe hybridizing directly to a sequence) or indirect (i.e., resulting from a probe hybridizing to an intermediate molecular structure that links the probe to the target sequence).

Hybridizing sequences will generally be more than about 50% complementary to all or a portion of any sequence in Tables 1 (RNA 1538, SEQ ID NOs:1-1546), 2, 2A (RNA143), 2B (RNA192), 3, 13 (SG175), 15 (SG128), or 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), or 18 (SG64). Typically, hybridizing sequences are 60%, 70%, 80%, 85%, 90%, or 95% complementary, or more to all or a portion of any sequence in Tables 1 (RNA 1538, SEQ ID NOs:1-1546), 2, 2A (RNA143), 2B (RNA192), 3, 13 (SG175), 15 (SG128), or 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), or 18 (SG64). The hybridization region between hybridizing sequences typically is at least about 5-10, 10-15 nucleotides, 15-20 nucleotides, 20-30 nucleotides, 30-50 nucleotides, 50-75 nucleotides, 75-100 nucleotides, 100-200 nucleotides, 300-400 nucleotides, 400-500 nucleotides or more, or any numerical value or range within or encompassing such lengths.

Hybridization between complementary regions of two strands of nucleic acid to form a duplex molecule will vary depending upon the nature of the hybridization method and the composition and length of the hybridizing nucleic acid sequences. Generally, temperature of hybridization and the ionic strength (such as the Na+ concentration) of the hybridization buffer will determine the stringency of hybridization (hybridization conditions for attaining particular degrees of stringency are discussed in Sambrook et al., (1989) Molecular Cloning, second edition, Cold Spring Harbor Laboratory, Plainview, N.Y.).

The following are exemplary non-limiting hybridization conditions: Very High Stringency (Detects Sequences that Share 90% Identity)—Hybridization: 5×SSC at 65° C. for 16 hours, Wash twice in 2×SSC at room temperature (RT) for 15 minutes each, Wash twice in 0.5×SSC at 65° C. for 20 minutes each.

High Stringency (Detects Sequences that Share 80% Identity or Greater)—Hybridization: 5-6×SSC at 65° C.-70° C. for 16-20 hours, Wash twice in 2×SSC at RT for 5-20 minutes each, Wash twice: 1×SSC at 55° C.-70° C. for 30 minutes each.
Low Stringency (Detects Sequences that Share Greater than 50% Identity)—Hybridization: 6×SSC at room temp. to 55° C. for 16-20 hours, Wash at least twice in 2-3×SSC at room temp. to 55° C. for 20-30 minutes each.

Accordingly, in various embodiments, polynucleotides and oligonucleotides (primers and probes) for hybridization include (e.g., contact) an oligo- or poly-nucleotide probe to an RNA transcript produced from a positive or negative predictor gene, or a polymorphism thereof, or hybridization of an oligo- or poly-nucleotide probe to a cDNA derived from the RNA transcript of a positive or negative predictor gene, or a polymorphism thereof. In a particular embodiment, polynucleotides and oligonucleotides (primers and probes) for hybridization include (e.g., contact) an oligo- or poly-nucleotide probe that binds to a positive or negative GVHD predictor gene sequence or a fragment thereof (e.g., to all or a portion of gene set forth in Tables 1 (RNA 1538, SEQ ID NOs:1-1546), 2, 2A (RNA143), 2B (RNA192), 3, 13 (SG175), 15 (SG128), or 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), or 18 (SG64). Such sequences therefore include fragments of the sequences in Tables 1 (RNA 1538, SEQ ID NOs:1-1546), 2, 2A (RNA143), 2B (RNA192), 3, 13 (SG175), 15 (SG128), and 18 (SG64), and sequences that are 50%, 60%, 70%, 80%, 85%, 90%, or 95% identical to all or a portion of any of the sequences in Tables 1 (RNA 1538, SEQ ID NOs:1-1546), 2, 2A (RNA143), 2B (RNA192), 3, 13 (SG175), 15 (SG128), and 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), or 18 (SG64).

A plurality of polynucleotides can be used in the invention methods, arrays and kits. Multiple polynucleotides (e.g., probes or primer pairs) can be used to detect, measure or analyze expression of a positive and/or negative predictor gene (e.g., any of the sequences in Tables 1 (RNA 1538, SEQ ID NOs:1-1546), 2, 2A (RNA143), 2B (RNA192), 3, 13 (SG175), 15 (SG128), and 18 (SG64), or a polymorphism thereof.

The term “complementary” or “antisense” refers to a polynucleotide or peptide nucleic acid (PNA) capable of binding to a specific DNA or RNA sequence, e.g, to all or a portion of any sequence in Tables 1 (RNA 1538, SEQ ID NOs:1-1546), 2, 2A (RNA143), 2B (RNA192), 3, 13 (SG175), 15 (SG128), and 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), and 18 (SG64) Antisense includes single, double, triple or greater stranded RNA and DNA polynucleotides and peptide nucleic acids (PNAs) that bind RNA transcript or DNA. Particular examples include RNA and DNA antisense that binds to sense RNA. For example, a single stranded nucleic acid can target a transcript of a negative and/or positive predictor gene. Antisense/Sense molecules are typically 100% complementary to the sense/anti-sense strand but can be “partially” complementary, in which only some of the nucleotides bind to the sense/anti-sense molecule (less than 100% complementary, e.g., 95%, 90%, 80%, 70% and sometimes less), or any numerical value or range within or encompassing such percent values.

Polynucleotides useful as primers and probes in invention methods, arrays and kits are typically a portion/fragment of a gene (sense or anti-sense) suitable for use as a hybridization probe or primer for the identification, detection, measurement or analysis of a gene (or portion/fragment thereof) in a given sample (e.g., a sample comprising CD4+ T cells or CD8+ T cells). Typically, primers are oppositely oriented, (i.e., one primer positioned 5′, and a second primer positioned 3′) such that they can hybridize to and amplify the nucleic acid sequence (e.g., via PCR).

Accordingly, in another embodiment, measuring includes hybridization of a primer pair (oppositely oriented) and subsequent amplification of a cDNA derived from the RNA transcript of the positive or negative GVHD predictor gene produced of the positive or negative predictor genes, or a polymorphism thereof (e.g., a gene set forth in any of Tables 1 (RNA 1538, SEQ ID NOs:1-1546), 2, 2A (RNA143), 2B (RNA192), 3, 13 (SG175), 15 (SG128), and 18 (SG64)). In a further embodiment, measuring includes reverse transcription of RNA transcript (e.g., using a primer pair, oppositely oriented) to produce cDNA to determine expression levels of one or more positive or negative GVHD predictor genes (e.g., a gene set forth in any of Tables 1 (RNA 1538, SEQ ID NOs:1-1546), 2, 2A (RNA143), 2B (RNA192), 3, 13 (SG175), 15 (SG128), and 18 (SG64)).

Nucleic acid sequences can include nucleotide and nucleoside substitutions, additions and deletions, derivatized forms and fusion/chimeric sequences (e.g., encoding recombinant polypeptide), as well as variants thereof (e.g., substitutions, additions insertions and deletions). Particular examples of such variants include polymorphisms and fragments of any sequence in Tables 1 (RNA 1538, SEQ ID NOs:1-1546), 2, 2A (RNA143), 2B (RNA192), 3, 13 (SG175), 15 (SG128), and 18 (SG64), or a sequence complementary to any sequence in Tables 1 (RNA 1538, SEQ ID NOs:1-1546), 2, 2A (RNA143), 2B (RNA192), 3, 13 (SG175), 15 (SG128), and 18 (SG64).

The term “identity” and grammatical variations thereof mean that two or more referenced entities are the same. Thus, where two sequences are identical, they have the same amino acid sequence. “Areas, regions or domains of identity” mean that a portion of two or more referenced entities are the same. Thus, where two sequences are identical or homologous over one or more sequence regions, they share identity in these regions.

The degree of “identity” and “homology” can be determined by comparing each position in the sequences. A degree of identity or homology is a function of the number of identical or matching positions (e.g., matching nucleotides or amino acid residues) at positions shared by the sequences. Specific examples of “identity” and “homology” include (e.g., 1-3, 3-5, 5-10, 10-20, 20-30, or more) residues of the sequences. A sequence can have 50%, 60%, 70%, 75%, 80%, 85%, 90%, 95%, 96%, 97%, 98%, 99%, or more identity or homology to a reference sequence, to all or a portion of any sequence in Tables 1 (RNA 1538, SEQ ID NOs:1-1546), 2, 2A (RNA143), 2B (RNA192), 3, 13 (SG175), 15 (SG128), and 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), and 18 (SG64). As used herein, a given percentage of identity or homology between sequences denotes the degree of sequence identity in optimally aligned sequences.

The extent of identity between two sequences can be ascertained using a computer program and mathematical algorithm. Such algorithms that calculate percent sequence identity (homology) generally account for sequence gaps and mismatches over the comparison region. For example, a BLAST (e.g., BLAST 2.0) search algorithm (see, e.g., Altschul et al., J. Mol. Biol. 215:403 (1990), publicly available through the National Center for Biotechnology Information, NCBI) has exemplary search parameters as follows: Mismatch-2; gap open 5; gap extension 2. The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance.

For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as eukaryote or mammalian cell, yeast or bacteria, in an animal or in a plant).

As disclosed herein, gene expression can be measured and/or analyzed by detection of an expression product. As used herein, the term “expression product” is an amino acid sequence, protein, polypeptide, or peptide encoded by a gene. In particular, an expression product, for example, is encoded by all or a part of a negative or positive GVHD predictor gene set forth in sequence in Tables 1 (RNA 1538, SEQ ID NOs:1-1546), 2, 2A (RNA143), 2B (RNA192), 3, 13 (SG175), 15 (SG128), or 18 (SG64). Invention methods, kits and arrays include detection, measurement or analysis of expression products encoded by one or more negative or positive GVHD predictor genes as set forth, for example, in Tables 1 (RNA 1538, SEQ ID NOs:1-1546), 2, 2A (RNA143), 2B (RNA192), 3, 13 (SG175), 15 (SG128), or 18 (SG64).

Accordingly, analytes further include molecules that bind to expression products, i.e., bind to amino acid sequence, protein, polypeptide, or peptide encoded by all or a part of a negative or positive GVHD predictor gene (e.g, a sequence set forth in any of Tables 1 (RNA 1538, SEQ ID NOs:1-1546), 2, 2A (RNA143), 2B (RNA192), 3, 13 (SG175), 15 (SG128), and 18 (SG64)). As used herein the terms “amino acid sequence,” “protein,” “polypeptide” and “peptide” are used interchangeably to refer to two or more amino acids, or “residues,” covalently linked by an amide bond or equivalent. Exemplary lengths of such amino acid sequences are from about 5 to 10, 10 to 20, 20 to 25, 25 to 50, 50 to 100, 100 to 150, 150 to 200, or 200 to 300, 400 to 500, 500 to 1000, or more amino acid residues in length.

Analytes according to the invention therefore include antibodies and subsequences thereof that bind to proteins or fragments (peptides, polypeptides, etc.) encoded by the positive or negative GVHD predictor genes. The term “antibody” refers to a protein that binds to other molecules (antigens) via heavy and/or light chain variable domains, V_{H} and/or V_{L}, respectively. An “antibody” refers to any monoclonal or polyclonal immunoglobulin molecule, such as IgG, IgA, IgD, IgE, IgM, and any subclass thereof (e.g., IgG₁, IgG₂, IgG₃ or IgG₄). Antibodies include full-length antibodies that include two heavy and two light chain sequences. Antibodies can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains.

Antibodies and subsequences thereof include mammalian, primatized, humanized and fully human antibodies and subsequences thereof. Antibodies and subsequences thereof include those produced or expressed by or on transformed cells or hybridomas, or B cells, or those produced synthetically or by other organisms (plant, insect, bacteria, etc.).

Antibodies include polyclonal and monoclonal antibodies. A “monoclonal” antibody refers to an antibody that is based upon, obtained from or derived from a single clone, including any eukaryotic, prokaryotic, or phage clone. A “monoclonal” antibody is therefore defined structurally, and not the method by which it is produced.

Antibodies include subsequences. Non-limiting representative antibody subsequences include but are not limited to Fab, Fab′, F(ab′)₂, Fv, Fd, single-chain Fv (scFv), disulfide-linked Fvs (sdFv), V_{L}, V_{H}, Camel Ig, V-NAR, VHH, trispecific (Fab₃), bispecific (Fab₂), diabody ((V_{L}-V_{H})₂ or (V_{H}-V_{L})₂), triabody (trivalent), tetrabody (tetravalent), minibody ((scF_{V}-C_{H}3)₂), bispecific single-chain Fv (Bis-scFv), IgGdeltaCH2, scFv-Fc, (scFv)₂-Fc, affibody, aptamer, avimer or nanobody, or other antigen binding subsequences of an intact immunoglobulin. Antibodies include those that bind to more than one epitope (e.g., bi-specific antibodies), or antibodies that can bind to one or more different antigens (e.g., bi- or multi-specific antibodies).

Antibodies and subsequences thereof can be produced or are available commercially or from other sources. For example, antibodies that bind to an expression produce or fragment encoded by all or a portion of any sequence in Tables 1 (RNA 1538), 2, 2A, 2B (RNA 192) and/or 3 can be produced using standard immunological methods known to one of skill in the art.

A mammalian antibody is an antibody produced by a mammal, transgenic or non-transgenic, or a non-mammalian organism engineered to produce a mammalian antibody, such as a non-mammalian cell (bacteria, yeast, insect cell), animal or plant. A “human” antibody means that the amino acid sequence of the antibody is fully human, i.e., human heavy and human light chain variable and human constant regions. Thus, all of the amino acids are human or exist in a human antibody. A “humanized” antibody, means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs.

Methods of measuring amounts of expression products encoded by negative and/or positive predictor genes are known to those of skill in the art. Non-limiting examples of protein detection, measurement and analysis methods include Western blot, immunoblot, enzyme-linked immunosorbant assay (ELISA), radioimmunoassay (RIA), immunoprecipitation, surface plasmon resonance, chemiluminescence, absorption, emission, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS) and flow cytometry. Amounts of expression products encoded by negative and positive predictor genes also include functional assays, based upon a function of the protein, such as enzyme or catalytic function, DNA binding function, ligand or receptor binding, signal transduction, etc.

The term “bind,” or “binding,” when used in reference to an analyte means that the binding moiety interacts at the molecular level with all or a part of a nucleic acid sequence or a gene expression product (e.g., protein). Specific binding is selective for the sequence or expression product. Specific and selective binding can be distinguished from non-specific binding using assays known in the art (e.g., immunoprecipitation, ELISA, flow cytometry, immunohistochemistry, Western blotting, nucleic acid hybridization, etc.).

An analyte can be labeled or tagged in order to be detectable. Detectable labels, markers and tags include labels suitable for gene expression or expression product detection, measurement, analysis and/or quantitation, and include any composition detectable by enzymatic, biochemical, spectroscopic, photochemical, immunochemical, isotopic, electrical, optical, chemical or other means. A detectable label can be attached (e.g., linked conjugated) to the analyte, or be within or be one or more atoms that comprise the analyte. As the structure of analytes can include one or more of carbon, hydrogen, nitrogen, oxygen, sulfur, phosphorous, etc., radioisotopes of any of carbon, hydrogen, nitrogen, oxygen, sulfur, phosphorous, etc., can be included within an analyte detectably labeled.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: C, N, O, H, S, Cu, Fe, Ga, Ti, Sr, Y, Tc, In, Pm, Gd, Sm, Ho, Lu, Re, At, Bi or Ac. In additional embodiments, a radioisotope can be one or more of: ³H, ¹¹C, ¹⁴C, ¹³N, ¹⁸O, ¹⁵C, ³²P, ³³P, ³⁵S, ¹²⁵I, or ¹³¹I.

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a colorimetric labels such as colloidal gold or colored glass or plastic (e.g., polystyrene, polypropylene, latex, etc.) beads; a fluorescent material or dye (e.g., umbelliferone, fluorescein, fluorescein isothiocyanate, rhodamine, dichlorotriazinylamine fluorescein, dansyl chloride, texas red, rhodamine); a luminescent material (e.g., luminol); or a bioluminescent material (e.g., green fluorescent protein, luciferase, luciferin, aequorin). A label can be any imaging agent that can be employed for gene expression or expression product detection, measurement, analysis and/or quantitation (e.g., for computed axial tomography (CAT or CT), fluoroscopy, single photon emission computed tomography (SPECT) imaging, optical imaging, positron emission tomography (PET), magnetic resonance imaging (MRI), gamma imaging).

A detectable label can also be linked or conjugated (e.g., covalently) to the analyte. In various embodiments a detectable label, such as a radionuclide or metal or metal oxide can be bound or conjugated to the analyte, either directly or indirectly. A linker or an intermediary functional group can be used to link an analyte to a detectable label.

The terms “fusion” or “chimeric” or “conjugate” and grammatical variations thereof, when used in reference to a molecule, means that the molecule contains portions or sections that are derived from, obtained or isolated from, or are based upon or modeled after two different molecular entities that are distinct from each other as they do not typically exist together in nature. That is, for example, one portion of an analyte fusion or conjugate includes or consists of a portion (e.g., antibody) that binds to a gene product (encoded by a positive or negative predictor gene), and a second portion that includes or consists of a detectable moiety or agent, each of first and second portions structurally distinct.

Fusions, chimers and conjugates can be linked indirectly or directly, by a covalent or by a non-covalent bond. Non-limiting examples of covalent bonds are amide bonds, non-natural and non-amide chemical bonds, which include, for example, glutaraldehyde, N-hydroxysuccinimide esters, bifunctional maleimides, N, N′-dicyclohexykarbodiimide (DCC) or N,N′-diisopropylcarbodiimide (DIC) Linking groups alternative to amide bonds include, for example, ketomethylene (e.g., —C(═O)—CH₂— for —C(═O)—NH—), aminomethylene (CH₂—NH₂), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide and Backbone Modifications,” Marcel Decker, NY).

Compositions and methods of the invention may be contacted or provided in vitro, ex vivo or in vivo. The term “contact” and grammatical variations thereof means conditions allowing a physical interaction (direct or indirect) between two or more entitites (e.g., an analyte and nucleic acid or expression product). In one example, contact means interaction (e.g., binding) of an analyte (e.g., polynucleotide, probe, primer, antibody or fragment, etc.) and a biological sample, such as CD4+ T cells, CD8+ T cells, or a cellular or other material derived from a biological sample, such as nucleic acid, protein, etc.

For methods of the invention for detection, measurement or analysis of expression, contact as used herein includes in solution, in solid phase, in situ, in vitro, ex vivo, in a cell, such as a sample that includes CD4+ cells or CD8+ T cells in vivo, in vitro, in primary cell isolates, passaged cells, cultured cells, or cells ex vivo. Thus, methods of the invention include contact under conditions allowing the analyte to bind to another entity indicative of positive and/or negative predictor gene expression amounts or levels.

An analyte (i.e., the nucleic acid, protein, antibody or fragment thereof) can be either in a free state, in solution or in solid phase, such as immobilized on a substrate or a support (e.g., solid). Examples of substrates and supports include a multiwall plate, a bead or sphere, a tube or vial, a microarray or any other suitable substrate or support. Immobilization can be by passive adsorption (non-covalent binding) or covalent binding between the substrate or support and the analyte, or indirectly by attaching the analyte to a reagent which reagent is then attached to the substrate or support (e.g., a ligand-receptor system, for example, where a molecule is grafted onto the analyte and the corresponding receptor immobilized on the substrate or support, as exemplified by the biotin-streptavidin system).

The term “bind,” or “binding,” means a physical interaction at the molecular level (directly or indirectly). Typically, binding is that which is specific or selective for a target, i.e., is statistically significantly higher than the background or control binding for the assay. The term “specifically binds” refers to the ability to preferentially or selectively bind to a target, for example, an analyte such as a polynucleotide, primer, probe, or antibody that binds to (or hybridizes with) a nucleic acid or gene expression product. Specific and selective binding can be distinguished from non-specific binding using assays known in the art (e.g., for nucleic acid detection, polymerase chain reaction, DNA transcription, northern and southern blotting, etc., and or protein detection, immunoprecipitation, ELISA, flow cytometry, and Western blotting). For example, when performing an immunoassay, controls typically include a reaction well/tube that contains an antibody or antigen binding fragment alone (i.e., in the absence of protein sample), wherein an amount of reactivity (e.g., non-specific binding to the well) by the antibody or antigen binding fragment thereof in the absence of protein sample is considered to be background.

The invention further provides databases and organizational constructs. A “database” or “organizational construct” refers to a collection of information. A database or organizational construct typically includes a gene expression profile of one or more actual and/or candidate HCT donors, or a score or other indicia that indicates the risk or probability of HCT from an actual and/or a candidate donor to induce or to not induce GVHD in a HCT recipient. In one embodiment, a database or organizational construct includes a gene expression profile of a plurality of positive and/or negative predictor genes of an actual or a candidate HCT donor, or a score that indicates the risk or probability of HCT from an actual or a candidate donor to induce or to not induce GVHD in a HCT recipient. In another embodiment, a database or organizational construct includes a gene expression profile of a plurality of positive and/or negative predictor genes of a plurality of an actual or a candidate donor HCT, or a score that indicates the risk or probability of HCT from a plurality of actual or candidate donors to induce or to not induce GVHD in a HCT recipient.

The risk of HCT of a given actual or candidate donor inducing GVHD can be used to anticipate whether, and to what extent (e.g., severity) that GVHD is induced in a HCT recipient. For example, if there are limited compatible HCT donors available for a given HCT recipient, a donor HCT that has some risk of inducing GVHD can be selected for the HCT recipient. Given that GVHD may be anticipated after transplant into the recipient, the recipient can be treated with an effective amount of an anti-rejection agent either prior to or following introduction of HCT into the recipient. Depending on the risk of inducing GVHD, the recipient may be a treated more or less aggressively based upon the anticipated risk, or it may be determined that the recipient can be treated according to a standard protocol. An HCT recipient so treated, can have complications associated with transplantation such as GVHD reduced or prevented. Accordingly, the invention provides methods in which risk of GVHD is anticipated in a HCT recipient, and such recipients can be treated with an anti-GVHD rejection-amelioration therapy, either prior to or following introduction of HCT into the recipient.

The invention provides kits, which kits include, for example, analytes, nucleic acid sequences, primers, probes, antibodies and arrays packaged into a suitable packaging material. Kit components can be used to detect, measure or analyze expression of positive and/or negative GVHD predictor genes (e.g., in Tables 1 (RNA 1538), 2, 2A, 2B (RNA 192) and/or 3), for example, a probe, primer pair or antibody that specifically binds to a positive or negative predictor gene (e.g., nucleic acid or expression product) of interest (e.g., a gene whose expression level correlates with risk of donor HCT inducing GVHD). Accordingly, in one embodiment, a kit includes an analyte, nucleic acid sequence, primer, probe, antibody or an array that allows detection, measurement or analysis of expression of one or more positive and/or negative GVHD predictor gene(s) set forth, for example, in Tables 1 (RNA 1538), 2, 2A, 2B (RNA 192) and/or 3, or an expression product encoded by any of such sequences.

The term “packaging material” refers to a physical structure housing one or more components of the kit. The packaging material can maintain the components sterilely, and can be made of material commonly used for such purposes (e.g., paper, corrugated fiber, glass, plastic, foil, ampules, vials, tubes, etc.). A kit can contain a plurality of components, e.g., two or more analytes alone or in combination.

A kit optionally includes a label or insert including a description of the components (type, amounts, doses, etc.), instructions for use in solid phase, in solution, in vitro, in situ, or in vivo, and any other components therein. Labels or inserts can include instructions for practicing any of the methods described herein. For example, instructions for measuring and/or analyzing gene expression to determine or predict risk of an actual or candidate donor HCT to induce or to not induce GVHD in a HCT recipient. The instructions can additionally indicate that a gene expression level greater than a predefined reference expression value (e.g., relative to a standard or a control), indicates a higher or lower risk of donor HCT inducing GVHD in a HCT recipient.

Labels or inserts can include information identifying manufacturer, lot numbers, manufacturer location and date, expiration dates. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Invention kits can additionally include a buffering agent, or a preservative or a stabilizing agent in a formulation containing an analyte (e.g., a nucleic acid sequence, primer, probe or antibody that allows detection, measurement or analysis of expression of a positive or negative GVHD predictor gene as set forth, for example, in Tables 1 (RNA 1538), 2, 2A, 2B (RNA 192) and/or 3, or an expression product encoded by any such sequences). Each component of the kit can be enclosed within an individual container and all of the various containers can be within a single package.

Kits of the invention can include nucleic acid(s) (e.g., oligonucleotides, primers, or probes) with 100% identity or 100% complementary to all or a portion of any gene sequence in Tables 1 (RNA 1538, SEQ ID NOs:1-1546), 2, 2A (RNA143), 2B (RNA192), 3, 13 (SG175), 15 (SG128), and 18 (SG64), as well as nucleic acid(s) (e.g., oligonucleotides, primers, or probes) having less than 100% identity or less than 100% complementary to all or a portion of a gene sequence in Tables 1 (RNA 1538), 2, 2A, 2B (RNA 192) and/or 3 (e.g., 60%, 70%, 80%, 85%, 90%, or 95% identity or complementary to all or a portion of any gene sequence in Tables 1 (RNA 1538, SEQ ID NOs:1-1546), 2, 2A (RNA143), 2B (RNA192), 3, 13 (SG175), 15 (SG128), and 18 (SG64)). Kits therefore include sense and/or anti-sense nucleic acid sequences that hybridize to all or a portion of positive or negative GVHD predictor gene sequences (or a polymorphism thereof) set forth in Tables 1 (RNA 1538, SEQ ID NOs:1-1546), 2, 2A (RNA143), 2B (RNA192), 3, 13 (SG175), 15 (SG128), and 18 (SG64), or a complementary sequence of all or a portion of gene sequences set forth in Tables 1 (RNA 1538, SEQ ID NOs:1-1546), 2, 2A (RNA143), 2B (RNA192), 3, 13 (SG175), 15 (SG128), and 18 (SG64). Such nucleic acid can be identical or complementary to all or a portion of a nucleic acid sequence in Tables 1 (RNA 1538, SEQ ID NOs:1-1546), 2, 2A (RNA143), 2B (RNA192), 3, 13 (SG175), 15 (SG128), and 18 (SG64).

In one embodiment, a kit includes two or more primer pairs (e.g., 3, 4, 5, 6, 7, 8, 9, 10, 11, 12, 13, 14, 15, 16, 17, 18, 19, 20, etc., or more, e.g., 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, etc., or more), each primer pair oppositely oriented to each other, and the primer pairs hybridize to RNA or cDNA produced from one of the positive or negative GVHD predictor genes listed in Tables 1 (RNA 1538, SEQ ID NOs:1-1546), 2, 2A (RNA143), 2B (RNA192), 3, 13 (SG175), 15 (SG128), or 18 (SG64), or to all or a portion of positive or negative GVHD predictor gene sequences (or a polymorphism thereof) set forth in Tables 1 (RNA 1538, SEQ ID NOs:1-1546), 2, 2A (RNA143), 2B (RNA192), 3, 13 (SG175), 15 (SG128), or 18 (SG64) or a complementary sequence of all or a portion of gene sequences set forth in Tables 1 (RNA 1538, SEQ ID NOs:1-1546), 2, 2A (RNA143), 2B (RNA192), 3, 13 (SG175), 15 (SG128), or 18 (SG64)

Kits of the invention can include other analytes. In one embodiment, a kit includes a probe that hybridizes to a nucleic acid sequence amplified by one of the primer pairs that hybridizes to all or a portion of positive or negative GVHD predictor gene sequences (or a polymorphism thereof) set forth in Tables 1 (RNA 1538, SEQ ID NOs:1-1546), 2, 2A (RNA143), 2B (RNA192), 3, 13 (SG175), 15 (SG128), or 18 (SG64), or a complementary sequence of all or a portion of gene sequences set forth in Tables 1 (RNA 1538, SEQ ID NOs:1-1546), 2, 2A (RNA143), 2B (RNA192), 3, 13 (SG175), 15 (SG128), or 18 (SG64), for example, RNA or cDNA of one or more of the positive or negative GVHD predictor genes (or a polymorphism thereof) listed in Tables 1 (RNA 1538, SEQ ID NOs:1-1546), 2, 2A (RNA143), 2B (RNA192), 3, 13 (SG175), 15 (SG128), or 18 (SG64). In particular aspects, a plurality of probes (e.g., 2, 3, 4, 5, 6, 7, 8, 9, 10, 11, 12, 13, 14, 15, 16, 17, 18, 19, 20, etc., or more, e.g., 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, etc., or more) that hybridize to all or a portion of positive or negative GVHD predictor gene sequences (or a polymorphism thereof) set forth in Tables 1 (RNA 1538, SEQ ID NOs:1-1546), 2, 2A (RNA143), 2B (RNA192), 3, 13 (SG175), 15 (SG128), or 18 (SG64), or a complementary sequence of all or a portion of such gene sequences, for example, RNA or cDNA of the positive or negative predictor genes listed in Tables 1 (RNA 1538, SEQ ID NOs:1-1546), 2, 2A (RNA143), 2B (RNA192), 3, 13 (SG175), 15 (SG128), or 18 (SG64).

Kits of the invention that include analytes need not have all or a portion of the analytes attached or affixed to a support or substrate. In one embodiment, a kit that includes primer pairs or probes, the primer pairs and/or probes are not attached or affixed to a support or substrate.

Kits of the invention can further include other reagents useful in assessing levels of expression of a nucleic acid (e.g., buffers and other reagents for performing PCR reactions, or for detecting binding of a probe to a nucleic acid). For example, a kit can also include additional useful materials and substances, such as a standard (e.g., a sample containing a known quantity of a nucleic acid to which expression results can be compared). Kits can additionally include a computer readable media (comprising, for example, a data analysis program, a reference gene expression profile, etc.), control samples, and other reagents for obtaining and/or processing sample and analysis, and analyzing gene expression data so obtained.

The invention provides arrays, which arrays include, for example, one or more analytes, nucleic acid sequences, polynucleotides, oligonucleotides, primers, probes or antibodies affixed to or contained in a support or substrate (e.g., such as a multi-well format, or a multi-well plate or dish). An “array” or “microarray,” which can also be referred to as a “bio-chip,” refers to an arrangement of binding (e.g., hybridizable) analytes, such as polynucleotides, oligonucleotides, primers, probes or antibodies, on a substrate. Such arrays are suitable for quantifying variations in gene expression levels, and are therefore useful for the methods described herein, for example, detecting, measuring or analyzing expression of one or more positive and/or negative predictor genes.

Typically, in an array an analyte (e.g, nucleic acid sequence, oligonucleotide, probe or antibody) that is a portion of a known gene (single strand, sense or anti-sense, e.g., of a positive or negative predictor gene) or that binds to a gene expression product (e.g., of a positive or negative predictor gene), occupies a defined or known address or location on a substrate or support. Accordingly, analytes, such as nucleic acid sequences, polynucleotides, oligonucleotides, primers, probes or antibodies can have a defined or known location, position or address on the support or substrate.

Analytes are typically arranged within two or more dimensions of the array. An array can assume different shapes. For example, the array can be regular (such as arranged in uniform rows and columns) or irregular. Thus, in ordered arrays the position/location of each sample is assigned to the sample at the time when it is applied to the array, and a key can correlate each position/location with the appropriate target. An ordered array can be arranged in a symmetrical grid pattern, but samples could be arranged in other patterns (such as in radially distributed lines, spiral lines, or ordered clusters). Arrays usually are computer readable, in that a computer can be programmed to correlate a particular address on the array with sample identity at that position (such as hybridization or binding data, including for instance signal intensity). In non-limiting examples of computer readable formats, the individual samples in the array are arranged regularly, for instance in a Cartesian grid pattern, which can be correlated to address information by a computer.

An array “format” includes any format in which an analyte can be affixed to or contained in the support or substrate, such as microtiter or multi-well plates or dishes, test tubes, inorganic sheets, dipsticks, etc. The particular format is unimportant. All that is necessary is that an analyte can be affixed to or contained in the support or substrate without affecting the functional behavior of the analyte absorbed thereon.

The support or substrate can be an inert material such as glass or plastic. One such material is an organic polymer such as polypropylene, which is chemically inert and hydrophobic, and has good chemical resistance to a variety of organic acids, organic agents, bases, salts, oxidizing agents, and mineral acids. Additional non-limiting examples include polyethylene, polybutylene, polyisobutylene, polybutadiene, polyisoprene, polyvinylpyrrolidine, polytetrafluroethylene, polyvinylidene difluoride, polyfluoroethylene-propylene, polyethylenevinyl alcohol, polymethylpentene, polycholorotrifluoroethylene, polysulfonones, hydroxylated biaxially oriented polypropylene, aminated biaxially oriented polypropylene, thiolated biaxially oriented polypropylene, etyleneacrylic acid, thylene methacrylic acid, nylons, and blends or copolymers thereof (e.g., blends of, alternating blocks of, or alternating components of, polypropylene, polyethylene, polybutylene, polyisobutylene, etc.).

In one embodiment, an array includes two or more primer pairs, wherein each primer pair is oppositely oriented to each other, and each of the primer pairs hybridize to all or a portion of any gene sequence (or a polymorphism thereof) in Tables 1 (RNA 1538, SEQ ID NOs:1-1546), 2, 2A (RNA143), 2B (RNA192), 3, 13 (SG175), 15 (SG128), or 18 (SG64), or a complementary sequence of all or a portion of any gene sequence set forth in Tables 1 (RNA 1538, SEQ ID NOs:1-1546), 2, 2A (RNA143), 2B (RNA192), 3, 13 (SG175), 15 (SG128), or 18 (SG64), and wherein each primer pair is affixed to or contained in a support or substrate. In particular aspects, one or more primers of a primer pair have 100% identity or 100% complementary to all or a portion of any gene sequence in Tables 1 (RNA 1538, SEQ ID NOs:1-1546), 2, 2A (RNA143), 2B (RNA192), 3, 13 (SG175), 15 (SG128), or 18 (SG64), or have less than 100% identity or less than 100% complementary to all or a portion of any gene sequence in Tables 1 (RNA 1538, SEQ ID NOs:1-1546), 2, 2A (RNA143), 2B (RNA192), 3, 13 (SG175), 15 (SG128), or 18 (SG64) (e.g., 60%, 70%, 80%, 85%, 90%, or 95% identity or complementary to all or a portion of any gene sequence in Tables 1 (RNA 1538, SEQ ID NOs:1-1546), 2, 2A (RNA143), 2B (RNA192), 3, 13 (SG175), 15 (SG128), or 18 (SG64)). In further particular aspects, the array further includes a probe (or a plurality of probes) that hybridizes to a nucleic acid sequence amplified by one of the primer pairs (e.g., all or a portion of any sequence in Tables 1 (RNA 1538, SEQ ID NOs:1-1546), 2, 2A (RNA143), 2B (RNA192), 3, 13 (SG175), 15 (SG128), or 18 (SG64), or a sequence complementary to all or a portion of any sequence in Tables 1 (RNA 1538, SEQ ID NOs:1-1546), 2, 2A (RNA143), 2B (RNA192), 3, 13 (SG175), 15 (SG128), or 18 (SG64).

In another embodiment, an array includes two or more probes, wherein each probe hybridizes to all or a portion of a gene sequence (or a polymorphism thereof) in Tables 1 (RNA 1538, SEQ ID NOs:1-1546), 2, 2A (RNA143), 2B (RNA192), 3, 13 (SG175), 15 (SG128), or 18 (SG64), or a complementary sequence of all or a portion of gene sequences set forth in Tables 1 (RNA 1538, SEQ ID NOs:1-1546), 2, 2A (RNA143), 2B (RNA192), 3, 13 (SG175), 15 (SG128), or 18 (SG64), and wherein each probe is affixed to or contained in a support or substrate. In particular aspects, one or more probes have 100% identity or is 100% complementary to all or a portion of a gene sequence in Tables 1 (RNA 1538, SEQ ID NOs:1-1546), 2, 2A (RNA143), 2B (RNA192), 3, 13 (SG175), 15 (SG128), or 18 (SG64), or has less than 100% identity or is less than 100% complementary to all or a portion of a gene sequence in Tables 1 (RNA 1538, SEQ ID NOs:1-1546), 2, 2A (RNA143), 2B (RNA192), 3, 13 (SG175), 15 (SG128), or 18 (SG64) (e.g., 60%, 70%, 80%, 85%, 90%, or 95% identity or complementary to all or a portion of a gene sequence in Tables 1 (RNA 1538), 2, 2A, 2B (RNA 192) and/or 3).

The hybridizing probe and/or primer sequence and sequence of the positive and negative predictor genes described herein are provided in sequence in Tables 1 (RNA 1538, SEQ ID NOs:1-1546), 2, 2A (RNA143), 2B (RNA192), 3, 13 (SG175), 15 (SG128), or 18 (SG64). Thus, knowing the sequence and identity of the genes set forth herein, nucleic acid and other analyte arrays can be fabricated either by de novo synthesis on a substrate or by spotting or transporting nucleic acid sequences onto specific locations of substrate. For example, nucleic acid purified and/or isolated from a biological material, such as a sample that includes CD4+ T cells or CD8+ T cells is hybridized with an array of such oligonucleotides or probes, and then the amount of target nucleic acid that hybridizes to each oligonucleotide or probe in the array can be determined. It is noted that all of the genes described herein have been previously sequenced, at least in part, such that oligonucleotides suitable for the detection, measurement and analysis of such genes can be produced.

In further embodiments, an array includes primers and/or probes that hybridize to 5, 10, 20, 30 or more of the positive or negative predictor genes (or a polymorphism thereof), or a complementary sequence of all or a portion of gene sequences (or a polymorphism thereof) set forth in Tables 1 (RNA 1538, SEQ ID NOs:1-1546), 2, 2A (RNA143), 2B (RNA192), 3, 13 (SG175), 15 (SG128), or 18 (SG64). In further embodiments, an array includes primers and/or probes all of which hybridize to all or a portion of a gene (or a polymorphism thereof) sequence in Tables 1 (RNA 1538, SEQ ID NOs:1-1546), 2, 2A (RNA143), 2B (RNA192), 3, 13 (SG175), 15 (SG128), or 18 (SG64), or a complementary sequence of all or a portion of gene sequences in Tables 1 (RNA 1538, SEQ ID NOs:1-1546), 2, 2A (RNA143), 2B (RNA192), 3, 13 (SG175), 15 (SG128), or 18 (SG64).

In still further embodiments, an array includes a total number of primer pairs and/or probes less than 30,000, less than 20,000, less than 15,000, less than 10,000, less than 5,000, less than 2,500, less than 2,000, less than 1,500, less than 1,000, less than 500, less than 400, less than 300, less than 200, less than 100, less than 50, or less than 25 primer pairs and/or probes.

By way of illustrative example only, an array of nucleic acids, polynucleotides, oligonucleotides, primers or probes, immobilized on the microchip, are suitable for hybridization to a nucleic acid sample. Fluorescently labeled cDNA probes (e.g., generated through incorporation of fluorescent nucleotides by reverse transcription of RNA extracted from CD4+ T cells or CD8+ T cells) are contacted or applied to the array, and allowed to hybridize with specificity to each spot of nucleic acid on the array. After washing to remove non-specifically bound cDNA probes, the array is scanned by a detection method (e.g., by confocal laser microscopy or a CCD camera). Quantitation of hybridization of each array element allows for assessment of mRNA abundance. With dual color fluorescence, separately labeled cDNA probes generated from two sources of RNA are hybridized pairwise to the array. The relative abundance of the transcripts from the two sources corresponding to each specified gene is thus determined. Such methods have been shown to have the sensitivity required to detect rare transcripts, expressed at a few copies per cell, and to reproducibly detect at least approximately two-fold differences in the expression levels (Schena et al., Proc. Natl. Acad. Sci. USA 93:106-149 (1996)).

Arrays can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known (a summary of suitable methods can be found in Matson et al., Anal. Biochem. 217:306-10 (1994)). In one example, oligonucleotides are synthesized onto the support using conventional chemical techniques for preparing oligonucleotides on solid supports (WO 85/01051, WO 89/10977, and U.S. Pat. No. 5,554,501).

The invention provides databases and organizational constructs. Databases and or organizational constructs can be operatively linked to a processor, such as a processor that includes a data entry module or a query module.

In one embodiment, a database or organizational construct includes gene expression profiles of two or more positive and/or negative predictor genes (e.g., from a biological sample of CD4+ T cells or CD8+ T cells) or a polymorphism thereof listed in Tables 1 (RNA 1538, SEQ ID NOs:1-1546), 2, 2A (RNA143), 2B (RNA192), 3, 13 (SG175), 15 (SG128), or 18 (SG64), from a plurality of actual or candidate HCT donors, and in which the gene expression profile is associated with each of the actual or candidate HCT donors in the database or organizational construct. In another embodiment, a database or organizational construct includes scores assigned based upon the probability or risk of actual or candidate donor HCTs to induce or to not induce GVHD in a HCT recipient, each of which score is based upon a gene expression profile of two or more positive and/or negative predictor genes or a polymorphism thereof listed in Tables 1 (RNA 1538), 2, 2A, 2B (RNA 192) and/or 3, for each actual or candidate HCT donor, and in which each score is associated with each of the actual or candidate HCT donors in the database or organizational construct. In particular aspects, the database or organizational construct includes expression information for 3, 4, 5, 6, 7, 8, 9, 10, 11, 12, 13, 14, 15, 16, 17, 18, 19, 20 or more, e.g., 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, etc., or more positive or negative predictor genes or a polymorphism thereof listed in Tables 1 (RNA 1538, SEQ ID NOs:1-1546), 2, 2A (RNA143), 2B (RNA192), 3, 13 (SG175), 15 (SG128), or 18 (SG64), for each actual or candidate HCT donor. In further particular aspects, the HCT from the actual or candidate donors at lower or higher risk of inducing graft vs. host disease (GVHD) in a HCT recipient are identified.

TABLE 1
1538 Positive and Negative Predictor Genes of GVHD Outcome, a Housekeeping “HSK” gene, and Exemplary Probes

| RNA1538 | RNA1546 Index | | | Accession No. Basic | | | |
|---|---|---|---|---|---|---|---|
| Index | (SEQ ID NO: 1-1546) | ProbeID | Accession No. | (without decimal) | Gene Name | Symbol | Synonyms |
| 1 | 1196 | 6280672 | NM_030938.2 | NM_030938 | transmembrane protein | TMEM49 | VMP1; |
| | | | | | 49 (TMEM49), mRNA. | | DKFZP566I133 |
| 2 | 12 | 4200575 | NM_014232.1 | NM_014232 | vesicle-associated | VAMP2 | SYB2; VAMP-2; |
| | | | | | membrane protein 2 | | FLJ11460 |
| | | | | | (synaptobrevin 2) | | |
| | | | | | (VAMP2), mRNA. | | |
| 3 | 461 | 830553 | NM_017455.2 | NM_017455 | neuroplastin (NPTN), | NPTN | SDFR1; GP55; |
| | | | | | transcript variant alpha, | | DKFZp686L2477; |
| | | | | | mRNA. | | np65; np55; GP65; |
| | | | | | | | SDR1; MGC102805 |
| 4 | 1256 | 6580711 | NM_001129.3 | NM_001129 | AE binding protein 1 | AEBP1 | FLJ33612; ACLP |
| | | | | | (AEBP1), mRNA. | | |
| 5 | 1309 | 6960594 | NM_145869.1 | NM_145869 | annexin A11 | ANXA11 | ANX11; CAP50 |
| | | | | | (ANXA11), transcript | | |
| | | | | | variant c, mRNA. | | |
| 6 | 224 | 6480095 | NM_030918.5 | NM_030918 | sorting nexin family | SNX27 | MGC126873; |
| | | | | | member 27 (SNX27), | | MGC20471; |
| | | | | | mRNA. | | MGC126871; MY014; |
| | | | | | | | KIAA0488 |
| 7 | 220 | 6400148 | NM_080430.2 | NM_080430 | selenoprotein M | SELM | MGC40146; SEPM |
| | | | | | (SELM), mRNA | | |
| 8 | 254 | 7610537 | NM_002129.2 | NM_002129 | high-mobility group box | HMGB2 | HMG2 |
| | | | | | 2 (HMGB2), mRNA. | | |
| 9 | 1535 | 6960278 | NM_178552.2 | NM_178552 | chromosome 22 open | C22orf33 | EAN57; MGC35206; |
| | | | | | reading frame 33 | | cE81G9.2 |
| | | | | | (C22orf33), mRNA. | | |
| 10 | 1067 | 5560133 | NM_152468.3 | NM_152468 | transmembrane | TMC8 | EVIN2; EVER2; EV2; |
| | | | | | channel-like 8 (TMC8), | | MGC40121; |
| | | | | | mRNA. | | MGC102701 |
| 11 | 508 | 1230017 | NM_018367.4 | NM_018367 | phytoceramidase, | PHCA | FLJ11238; APHC |
| | | | | | alkaline (PHCA), | | |
| | | | | | mRNA. | | |
| 12 | 754 | 2940075 | NM_018571.5 | NM_018571 | amyotrophic lateral | ALS2CR2 | ILPIPA; ILPIP; PAPK; |
| | | | | | sclerosis 2 (juvenile) | | MGC102916; CALS- |
| | | | | | chromosome region, | | 21; PRO1038 |
| | | | | | candidate 2 | | |
| | | | | | (ALS2CR2), mRNA. | | |
| 13 | 1197 | 6290021 | NM_002811.3 | NM_002811 | proteasome (prosome, | PSMD7 | P40; S12; MOV34 |
| | | | | | macropain) 26S | | |
| | | | | | subunit, non-ATPase, 7 | | |
| | | | | | (PSMD7), mRNA. | | |
| 14 | 406 | 540446 | NM_012459.1 | NM_012459 | translocase of inner | TIMM8B | MGC102866; TIM8B; |
| | | | | | mitochondrial | | DDP2; MGC117373; |
| | | | | | membrane 8 homolog | | FLJ21744 |
| | | | | | B (yeast) (TIMM8B), | | |
| | | | | | nuclear gene encoding | | |
| | | | | | mitochondrial protein, | | |
| | | | | | mRNA. | | |
| 15 | 658 | 2260296 | NR_003654.1 | NR_003654 | SCAN domain | SCAND2 | — |
| | | | | | containing 2 (SCAND2) | | |
| | | | | | on chromosome 15. | | |
| 16 | 172 | 4830113 | NM_016619.1 | NM_016619 | placenta-specific 8 | PLAC8 | C15; onzin |
| | | | | | (PLAC8), mRNA. | | |
| 17 | 1357 | 7380601 | NM_024896.2 | NM_024896 | endoplasmic reticulum | ERMP1 | FXNA; KIAA1815; |
| | | | | | metallopeptidase 1 | | bA207C16.3 |
| | | | | | (ERMP1), mRNA. | | |
| 18 | 757 | 2970397 | NM_145288.1 | NM_145288 | zinc finger protein 342 | ZNF342 | ZNF296 |
| | | | | | (ZNF342), mRNA. | | |
| 19 | 237 | 6960593 | NM_004439.4 | NM_004439 | EPH receptor A5 | EPHA5 | EHK1; TYRO4; |
| | | | | | (EPHA5), transcript | | HEK7; CEK7 |
| | | | | | variant 1, mRNA. | | |
| 20 | 1330 | 7150685 | NM_012117.1 | NM_012117 | chromobox homolog 5 | CBX5 | HP1-ALPHA; HP1; |
| | | | | | (HP1 alpha homolog, | | HP1Hs-alpha |
| | | | | | Drosophila) (CBX5), | | |
| | | | | | mRNA. | | |
| 21 | 570 | 1660072 | NM_130787.2 | NM_130787 | adaptor-related protein | AP2A1 | CLAPA1; AP2- |
| | | | | | complex 2, alpha 1 | | ALPHA; ADTAA |
| | | | | | subunit (AP2A1), | | |
| | | | | | transcript variant 2, | | |
| | | | | | mRNA. | | |
| 22 | 207 | 6060196 | NM_145912.5 | NM_145912 | NFAT activating protein | NFAM1 | FLJ40652; CNAIP; |
| | | | | | with ITAM motif 1 | | bK126B4.4 |
| | | | | | (NFAM1), mRNA. | | |
| 23 | 14 | 5220196 | NM_006565.2 | NM_006565 | CCCTC-binding factor | CTCF | — |
| | | | | | (zinc finger protein) | | |
| | | | | | (CTCF), mRNA. | | |
| 24 | 1329 | 7150278 | NM_000201.1 | NM_000201 | intercellular adhesion | ICAM1 | P3.58; BB2; CD54 |
| | | | | | molecule 1 (CD54), | | |
| | | | | | human rhinovirus | | |
| | | | | | receptor (ICAM1), | | |
| | | | | | mRNA. | | |
| 25 | 271 | 2510253 | NM_145306.2 | NM_145306 | chromosome 10 open | C10orf35 | — |
| | | | | | reading frame 35 | | |
| | | | | | (C10orf35), mRNA. | | |
| 26 | 457 | 830324 | NM_001459.2 | NM_001459 | fms-related tyrosine | FLT3LG | — |
| | | | | | kinase 3 ligand | | |
| | | | | | (FLT3LG), mRNA. | | |
| 27 | 1042 | 5290008 | NM_015112.2 | NM_015112 | microtubule associated | MAST2 | FLJ39200; RP4- |
| | | | | | serine/threonine kinase | | 533D7.1; KIAA0807; |
| | | | | | 2 (MAST2), mRNA. | | MAST205; MTSSK |
| 28 | 231 | 6650451 | NM_015057.3 | NM_015057 | MYC binding protein 2 | MYCBP2 | FLJ21597; PAM; |
| | | | | | (MYCBP2), mRNA. | | FLJ13826; FLJ10106; |
| | | | | | | | FLJ21646; |
| | | | | | | | DKFZp686M08244; |
| | | | | | | | KIAA0916 |
| 29 | 774 | 3140095 | NM_177543.1 | NM_177543 | phosphatidic acid | PPAP2C | PAP-2c; PAP2-g; |
| | | | | | phosphatase type 2C | | LPP2 |
| | | | | | (PPAP2C), transcript | | |
| | | | | | variant 3, mRNA. | | |
| 30 | 351 | 130241 | NM_001007468.1 | NM_001007468 | SWI/SNF related, | SMARCB1 | Sfh1p; RDT; hSNFS; |
| | | | | | matrix associated, actin | | SNF5; Snr1; SNF5L1; |
| | | | | | dependent regulator of | | INI1; BAF47 |
| | | | | | chromatin, subfamily b, | | |
| | | | | | member 1 | | |
| | | | | | (SMARCB1), transcript | | |
| | | | | | variant 2, mRNA. | | |
| 31 | 181 | 5090288 | NM_171999.2 | NM_171999 | sal-like 3 (Drosophila) | SALL3 | ZNF796 |
| | | | | | (SALL3), mRNA. | | |
| 32 | 272 | 2810082 | NM_016470.6 | NM_016470 | chromosome 20 open | C20orf111 | dJ1183I21.1; |
| | | | | | reading frame 111 | | HSPC207; Perit1 |
| | | | | | (C20orf111), mRNA. | | |
| 33 | 1084 | 5690333 | NM_003400.3 | NM_003400 | exportin 1 (CRM1 | XPO1 | DKFZp686B1823; |
| | | | | | homolog, yeast) | | CRM1 |
| | | | | | (XPO1), mRNA. | | |
| 34 | 1316 | 7000735 | NM_002882.2 | NM_002882 | RAN binding protein 1 | RANBP1 | MGC88701 |
| | | | | | (RANBP1), mRNA. | | |
| 35 | 1369 | 7510687 | NM_006662.2 | NM_006662 | Snf2-related CREBBP | SRCAP | EAF1; SWR1; |
| | | | | | activator protein | | DOMO1; KIAA0309; |
| | | | | | (SRCAP), mRNA. | | FLJ44499 |
| 36 | 1085 | 5690358 | NM_014254.1 | NM_014254 | transmembrane protein | TMEM5 | HP10481 |
| | | | | | 5 (TMEM5), mRNA. | | |
| 37 | 1187 | 6270020 | NM_145799.2 | NM_145799 | septin 6 (SEPT6), | SEPT6 | SEP2; RP5- |
| | | | | | transcript variant I, | | 876A24.2; |
| | | | | | mRNA. | | MGC16619; SEPT2; |
| | | | | | | | MGC20339; |
| | | | | | | | KIAA0128 |
| 38 | 59 | 1170332 | NM_014911.3 | NM_014911 | AP2 associated kinase | AAK1 | DKFZp686K16132; |
| | | | | | 1 (AAK1), mRNA. | | MGC164568; |
| | | | | | | | FLJ45252; FLJ23712; |
| | | | | | | | FLJ25931; KIAA1048; |
| | | | | | | | FLJ42882; |
| | | | | | | | DKFZp686F03202; |
| | | | | | | | MGC164570; |
| | | | | | | | FLJ31060; |
| | | | | | | | MGC138170 |
| 39 | 736 | 2750184 | NM_005184.2 | NM_005184 | calmodulin 3 | CALM3 | PHKD; PHKD3 |
| | | | | | (phosphorylase kinase, | | |
| | | | | | delta) (CALM3), mRNA. | | |
| 40 | 1078 | 5570601 | NM_020216.3 | NM_020216 | arginylaminopeptidase | RNPEP | DKFZP547H084 |
| | | | | | (aminopeptidase B) | | |
| | | | | | (RNPEP), mRNA. | | |
| 41 | 374 | 360280 | NM_017761.2 | NM_017761 | proline-rich nuclear | PNRC2 | MGC99541; |
| | | | | | receptor coactivator 2 | | FLJ20312 |
| | | | | | (PNRC2), mRNA. | | |
| 42 | 1281 | 6760347 | NM_178009.2 | NM_178009 | diacylglycerol kinase, | DGKH | DKFZp761I1510; |
| | | | | | eta (DGKH), transcript | | DGKeta |
| | | | | | variant 2, mRNA. | | |
| 43 | 160 | 4230619 | NM_012198.2 | NM_012198 | grancalcin, EF-hand | GCA | GCL |
| | | | | | calcium binding protein | | |
| | | | | | (GCA), mRNA. | | |
| 44 | 459 | 830463 | NM_002735.1 | NM_002735 | protein kinase, cAMP- | PRKAR1B | PRKAR1 |
| | | | | | dependent, regulatory, | | |
| | | | | | type I, beta | | |
| | | | | | (PRKAR1B), mRNA. | | |
| 45 | 353 | 130364 | NM_032947.3 | NM_032947 | MSTP150 (MST150), | MST150 | NID67; MGC126887; |
| | | | | | mRNA. | | MGC126889; |
| | | | | | | | MGC117221 |
| 46 | 52 | 1050408 | NM_005678.3 | NM_005678 | SNRPN upstream | SNURF | — |
| | | | | | reading frame | | |
| | | | | | (SNURF), transcript | | |
| | | | | | variant 1, mRNA. | | |
| 47 | 58 | 1170300 | NM_005950.1 | NM_005950 | metallothionein 1G | MT1G | MT1; MT1K; |
| | | | | | (MT1G), mRNA. | | MGC12386 |
| 48 | 23 | 20056 | NM_003295.1 | NM_003295 | tumor protein, | TPT1 | TCTP; p02; HRF; |
| | | | | | translationally- | | FLJ27337 |
| | | | | | controlled 1 (TPT1), | | |
| | | | | | mRNA. | | |
| 49 | 850 | 3800309 | NM_022170.1 | NM_022170 | eukaryotic translation | EIF4H | KIAA0038; WSCR1; |
| | | | | | initiation factor 4H | | WBSCR1 |
| | | | | | (EIF4H), transcript | | |
| | | | | | variant 1, mRNA. | | |
| 50 | 1 | 380575 | NM_000978.3 | NM_000978 | ribosomal protein L23 | RPL23 | MGC117346; rpL17; |
| | | | | | (RPL23), mRNA. | | MGC111167; |
| | | | | | | | MGC72008 |
| 51 | 2 | 940398 | NM_006360.3 | NM_006360 | eukaryotic translation | EIF3M | FLJ29030; GA17; hfl- |
| | | | | | initiation factor 3, | | B5; eIF3m; PCID1; B5 |
| | | | | | subunit M (EIF3M), | | |
| | | | | | mRNA. | | |
| 52 | 3 | 990315 | NM_030752.2 | NM_030752 | t-complex 1 (TCP1), | TCP1 | TCP-1-alpha; CCT- |
| | | | | | transcript variant 1, | | alpha; CCT1; |
| | | | | | mRNA. | | D6S230E; CCTa |
| 53 | 4 | 1240136 | NM_199345.3 | NM_199345 | phosphatidylinositol 4- | PI4KAP2 | FLJ44912; |
| | | | | | kinase, catalytic, alpha | | MGC31920 |
| | | | | | polypeptide | | |
| | | | | | pseudogene 2 | | |
| | | | | | (PI4KAP2), mRNA. | | |
| 54 | 5 | 1820482 | NM_004548.1 | NM_004548 | NADH dehydrogenase | NDUFB10 | PDSW |
| | | | | | (ubiquinone) 1 beta | | |
| | | | | | subcomplex, 10, 22 kDa | | |
| | | | | | (NDUFB10), mRNA. | | |
| 55 | 6 | 1850288 | NM_014153.2 | NM_014153 | zinc finger CCCH-type | ZC3H7A | HSPC055; |
| | | | | | containing 7A | | ZC3HDC7; ZC3H7; |
| | | | | | (ZC3H7A), mRNA. | | FLJ20318; FLJ10027 |
| 56 | 7 | 2940022 | NM_000712.3 | NM_000712 | biliverdin reductase A | BLVRA | BVRA; BLVR |
| | | | | | (BLVRA), mRNA. | | |
| 57 | 8 | 3370164 | NM_000701.6 | NM_000701 | ATPase, Na+/K+ | ATP1A1 | MGC3285; |
| | | | | | transporting, alpha 1 | | MGC51750 |
| | | | | | polypeptide (ATP1A1), | | |
| | | | | | transcript variant 1, | | |
| | | | | | mRNA. | | |
| 58 | 9 | 3440400 | NM_020698.1 | NM_020698 | transmembrane and | TMCC3 | KIAA1145 |
| | | | | | coiled-coil domain | | |
| | | | | | family 3 (TMCC3), | | |
| | | | | | mRNA. | | |
| 59 | 10 | 3450148 | NM_170734.2 | NM_170734 | brain-derived | BDNF | MGC34632 |
| | | | | | neurotrophic factor | | |
| | | | | | (BDNF), transcript | | |
| | | | | | variant 6, mRNA. | | |
| 60 | 11 | 3780450 | NM_079837.2 | NM_079837 | BTG3 associated | BANP | DKFZp761H172; |
| | | | | | nuclear protein (BANP), | | FLJ10177; SMAR1; |
| | | | | | transcript variant 2, | | SMARBP1; FLJ20538 |
| | | | | | mRNA. | | |
| 61 | 13 | 4640689 | NM_001967.3 | NM_001967 | eukaryotic translation | EIF4A2 | DDX2B; BM-010; |
| | | | | | initiation factor 4A, | | EIF4A; EIF4F |
| | | | | | isoform 2 (EIF4A2), | | |
| | | | | | mRNA. | | |
| 62 | 15 | 5870632 | NM_004800.1 | NM_004800 | transmembrane 9 | TM9SF2 | P76; MGC117391; |
| | | | | | superfamily member 2 | | FLJ26287 |
| | | | | | (TM9SF2), mRNA. | | |
| 63 | 16 | 6290392 | NM_005839.3 | NM_005839 | serine/arginine | SRRM1 | SRM160; 160-KD; |
| | | | | | repetitive matrix 1 | | POP101; MGC39488 |
| | | | | | (SRRM1), mRNA. | | |
| 64 | 17 | 6380008 | NM_025209.2 | NM_025209 | enhancer of polycomb | EPC1 | Epl1; |
| | | | | | homolog 1 (Drosophila) | | DKFZp781P2312 |
| | | | | | (EPC1), mRNA. | | |
| 65 | 18 | 6380427 | NM_202468.1 | NM_202468 | GIPC PDZ domain | GIPC1 | IIP-1; TIP-2; |
| | | | | | containing family, | | GLUT1CBP; C19orf3; |
| | | | | | member 1 (GIPC1), | | RGS19IP1; Hs.6454; |
| | | | | | transcript variant 3, | | SYNECTIIN; |
| | | | | | mRNA. | | MGC15889; NIP; |
| | | | | | | | MGC3774; SEMCAP; |
| | | | | | | | GIPC |
| 66 | 19 | 6580553 | NM_005688.2 | NM_005688 | ATP-binding cassette, | ABCC5 | MOAT-C; pABC11; |
| | | | | | sub-family C | | ABC33; MRP5; |
| | | | | | (CFTR/MRP), member | | SMRP; EST277145; |
| | | | | | 5 (ABCC5), transcript | | DKFZp686C1782; |
| | | | | | variant 1, mRNA. | | MOATC |
| 67 | 20 | 7210128 | NM_024408.2 | NM_024408 | Notch homolog 2 | NOTCH2 | hN2; AGS2 |
| | | | | | (Drosophila) | | |
| | | | | | (NOTCH2), mRNA. | | |
| 68 | 21 | 10504 | NM_031950.2 | NM_031950 | fibroblast growth factor | FGFBP2 | KSP37 |
| | | | | | binding protein 2 | | |
| | | | | | (FGFBP2), mRNA. | | |
| 69 | 22 | 20010 | NM_001014438.1 | NM_001014438 | cysteinyl- | CARS | CARS1; CYSRS; |
| | | | | | tRNAsynthetase | | MGC: 11246 |
| | | | | | (CARS), transcript | | |
| | | | | | variant 4, mRNA. | | |
| 70 | 24 | 60053 | NM_000975.2 | NM_000975 | ribosomal protein L11 | RPL11 | GIG34 |
| | | | | | (RPL11), mRNA. | | |
| 71 | 25 | 60397 | NR_001449.1 | NR_001449 | tRNA lysine 1 (TRK1) | TRK1 | — |
| | | | | | on chromosome 17. | | |
| 72 | 26 | 70008 | NM_000433.2 | NM_000433 | neutrophil cytosolic | NCF2 | p67phox; NOXA2; |
| | | | | | factor 2 (65 kDa, chronic | | P67-PHOX |
| | | | | | granulomatous | | |
| | | | | | disease, autosomal 2) | | |
| | | | | | (NCF2), mRNA. | | |
| 73 | 27 | 270544 | NM_003297.1 | NM_003297 | nuclear receptor | NR2C1 | TR2-11; TR2 |
| | | | | | subfamily 2, group C, | | |
| | | | | | member 1 (NR2C1), | | |
| | | | | | transcript variant 1, | | |
| | | | | | mRNA. | | |
| 74 | 28 | 450195 | NM_001788.4 | NM_001788 | septin 7 (SEPT7), | SEPT7 | Nbla02942; CDC10; |
| | | | | | transcript variant 1, | | SEPT7A; CDC3 |
| | | | | | mRNA. | | |
| 75 | 29 | 450431 | NM_199424.1 | NM_199424 | WW domain containing | WWP2 | WWp2-like; AIP2 |
| | | | | | E3 ubiquitin protein | | |
| | | | | | ligase 2 (WWP2), | | |
| | | | | | transcript variant 2, | | |
| | | | | | mRNA. | | |
| 76 | 30 | 450615 | NM_005953.2 | NM_005953 | metallothionein 2A | MT2A | MT2 |
| | | | | | (MT2A), mRNA. | | |
| 77 | 31 | 450762 | NM_021642.2 | NM_021642 | Fc fragment of IgG, low | FCGR2A | FCGR2A1; CDw32; |
| | | | | | affinity IIa, receptor | | CD32A; CD32; FcGR; |
| | | | | | (CD32) (FCGR2A), | | FCG2; IGFR2; |
| | | | | | mRNA. | | FCGR2; MGC30032; |
| | | | | | | | MGC23887 |
| 78 | 32 | 460411 | NM_006390.2 | NM_006390 | importin 8 (IPO8), | IPO8 | FLJ26580; RANBP8 |
| | | | | | mRNA. | | |
| 79 | 33 | 460750 | NM_024065.3 | NM_024065 | phosducin-like 3 | PDCL3 | VIAF1; HTPHLP; |
| | | | | | (PDCL3), mRNA. | | MGC3062 |
| 80 | 34 | 520133 | NM_001005849.1 | NM_001005849 | SMT3 suppressor of | SUMO2 | SMT3H2; HSMT3; |
| | | | | | mif two 3 homolog 2 (S. cerevisiae) | | MGC117191; SMT3B |
| | | | | | (SUMO2), | | |
| | | | | | transcript variant 2, | | |
| | | | | | mRNA. | | |
| 81 | 35 | 520392 | NM_023914.2 | NM_023914 | purinergic receptor | P2RY13 | P2Y13; FKSG77; |
| | | | | | P2Y, G-protein | | SP174; GPR94; |
| | | | | | coupled, 13 (P2RY13), | | GPCR1; GPR86 |
| | | | | | transcript variant 1, | | |
| | | | | | mRNA. | | |
| 82 | 36 | 580255 | NM_001624.2 | NM_001624 | absent in melanoma 1 | AIM1 | ST4 |
| | | | | | (AIM1), mRNA. | | |
| 83 | 37 | 610014 | NM_003541.2 | NM_003541 | histone cluster 1, H4k | HIST1H4K | dJ160A22.1; H4/d; |
| | | | | | (HIST1H4K), mRNA. | | H4FD; H4F2iii |
| 84 | 38 | 610309 | NM_207115.1 | NM_207115 | zinc finger protein 580 | ZNF580 | — |
| | | | | | (ZNF580), transcript | | |
| | | | | | variant 2, mRNA. | | |
| 85 | 39 | 610670 | NM_145805.1 | NM_145805 | ISL LIM homeobox 2 | ISL2 | FLJ10160 |
| | | | | | (ISL2), mRNA. | | |
| 86 | 40 | 620047 | NM_004331.2 | NM_004331 | BCL2/adenovirus E1B | BNIP3L | BNIP3a; NIX |
| | | | | | 19 kDa interacting | | |
| | | | | | protein 3-like (BNIP3L), | | |
| | | | | | mRNA. | | |
| 87 | 41 | 630403 | NM_005830.2 | NM_005830 | mitochondrial ribosomal | MRPS31 | MRP-S31; IMOGN38 |
| | | | | | protein S31 (MRPS31), | | |
| | | | | | nuclear gene encoding | | |
| | | | | | mitochondrial protein, | | |
| | | | | | mRNA. | | |
| 88 | 42 | 630706 | NM_144653.3 | NM_144653 | BTB (POZ) domain | BTBD14A | BTBD14; MGC23427 |
| | | | | | containing 14A | | |
| | | | | | (BTBD14A), mRNA. | | |
| 89 | 43 | 670255 | NM_001924.2 | NM_001924 | growth arrest and DNA- | GADD45A | GADD45; DDIT1 |
| | | | | | damage-inducible, | | |
| | | | | | alpha (GADD45A), | | |
| | | | | | mRNA. | | |
| 90 | 44 | 780603 | NR_002305.1 | NR_002305 | protein disulfide | PDIA3P | ERp60; GRP58P |
| | | | | | isomerase family A, | | |
| | | | | | member 3 pseudogene | | |
| | | | | | (PDIA3P) on | | |
| | | | | | chromosome 1. | | |
| 91 | 45 | 830041 | NM_001005193.1 | NM_001005193 | olfactory receptor, | OR7G2 | OST260; OR19-6 |
| | | | | | family 7, subfamily G, | | |
| | | | | | member 2 (OR7G2), | | |
| | | | | | mRNA. | | |
| 92 | 46 | 830619 | NM_004083.4 | NM_004083 | DNA-damage-inducible | DDIT3 | MGC4154; CEBPZ; |
| | | | | | transcript 3 (DDIT3), | | CHOP10; CHOP; |
| | | | | | mRNA. | | GADD153 |
| 93 | 47 | 870082 | NM_012402.2 | NM_012402 | ADP-ribosylation factor | ARFIP2 | POR1 |
| | | | | | interacting protein 2 | | |
| | | | | | (arfaptin 2) (ARFIP2), | | |
| | | | | | mRNA. | | |
| 94 | 48 | 990056 | NM_020706.1 | NM_020706 | splicing factor, | SFRS15 | SCAF4; |
| | | | | | arginine/serine-rich 15 | | DKFZP434E098; |
| | | | | | (SFRS15), mRNA. | | FLJ23364; SRA4; |
| | | | | | | | KIAA1172 |
| 95 | 49 | 990273 | NM_000998.4 | NM_000998 | ribosomal protein L37a | RPL37A | MGC74786 |
| | | | | | (RPL37A), mRNA. | | |
| 96 | 50 | 990543 | NM_004768.2 | NM_004768 | splicing factor, | SFRS11 | DKFZp686M13204; |
| | | | | | arginine/serine-rich 11 | | dJ677H15.2; p54 |
| | | | | | (SFRS11), mRNA. | | |
| 97 | 51 | 1030431 | NM_001995.2 | NM_001995 | acyl-CoA synthetase | ACSL1 | FACL2; LACS; |
| | | | | | long-chain family | | FACL1; ACS1; |
| | | | | | member 1 (ACSL1), | | LACS2; LACS1 |
| | | | | | mRNA. | | |
| 98 | 53 | 1050762 | NM_003844.2 | NM_003844 | tumor necrosis factor | TNFRSF10A | TRAILR1; MGC9365; |
| | | | | | receptor superfamily, | | APO2; DR4; CD261; |
| | | | | | member 10a | | TRAILR-1 |
| | | | | | (TNFRSF10A), mRNA. | | |
| 99 | 54 | 1070373 | NM_001012994.1 | NM_001012994 | sorting nexin family | SNX30 | FLJ35589; FLJ46877; |
| | | | | | member 30 (SNX30), | | FLJ45069; FLJ26481; |
| | | | | | mRNA. | | FLJ44686; FLJ34280 |
| 100 | 55 | 1070435 | NM_201433.1 | NM_201433 | growth arrest-specific 7 | GAS7 | MGC1348; |
| | | | | | (GAS7), transcript | | MLL/GAS7; |
| | | | | | variant c, mRNA. | | KIAA0394 |
| 101 | 56 | 1070593 | NM_007246.2 | NM_007246 | kelch-like 2, Mayven | KLHL2 | ABP-KELCH; MAV; |
| | | | | | (Drosophila) (KLHL2), | | MAYVEN |
| | | | | | mRNA. | | |
| 102 | 57 | 1090474 | NM_000073.1 | NM_000073 | CD3g molecule, | CD3G | MGC138597; CD3- |
| | | | | | gamma (CD3-TCR | | GAMMA; T3G |
| | | | | | complex) (CD3G), | | |
| | | | | | mRNA. | | |
| 103 | 60 | 1230292 | NM_080651.1 | NM_080651 | mediator complex | MED30 | TRAP25; MGC9890; |
| | | | | | subunit 30 (MED30), | | MED30; THRAP6 |
| | | | | | mRNA. | | |
| 104 | 61 | 1240064 | NM_012482.3 | NM_012482 | zinc finger protein 281 | ZNF281 | FLJ12859; ZNP-99; |
| | | | | | (ZNF281), mRNA. | | ZBP-99; FLJ14378 |
| 105 | 62 | 1240142 | NM_017654.2 | NM_017654 | sterile alpha motif | SAMD9 | KIAA2004; C7orf5; |
| | | | | | domain containing 9 | | OEF1; FLJ20073; |
| | | | | | (SAMD9), mRNA. | | NFTC; OEF2 |
| 106 | 63 | 1240192 | NM_001319.5 | NM_001319 | casein kinase 1, | CSNK1G2 | CK1g2 |
| | | | | | gamma 2 (CSNK1G2), | | |
| | | | | | mRNA. | | |
| 107 | 64 | 1260136 | NM_001080497.1 | NM_001080497 | multiple EGF-like- | MEGF9 | EGFL5 |
| | | | | | domains 9 (MEGF9), | | |
| | | | | | mRNA. | | |
| 108 | 65 | 1340537 | NM_001001655.1 | NM_001001655 | alkB, alkylation repair | ALKBH2 | ABH2; MGC90512; |
| | | | | | homolog 2 (E. coli) | | hABH2 |
| | | | | | (ALKBH2), mRNA. | | |
| 109 | 66 | 1410068 | NM_019884.2 | NM_019884 | glycogen synthase | GSK3A | DKFZp686D0638 |
| | | | | | kinase 3 alpha | | |
| | | | | | (GSK3A), mRNA. | | |
| 110 | 67 | 1410168 | NM_001421.2 | NM_001421 | E74-like factor 4 (ets | ELF4 | MEF; ELFR |
| | | | | | domain transcription | | |
| | | | | | factor) (ELF4), mRNA. | | |
| 111 | 68 | 1410221 | NM_005621.1 | NM_005621 | S100 calcium binding | S100A12 | CAAF1; CAGC; |
| | | | | | protein A12 (S100A12), | | ENRAGE; p6; CGRP; |
| | | | | | mRNA. | | MRP6 |
| 112 | 69 | 1410411 | NM_182710.1 | NM_182710 | HIV-1 Tat interacting | HTATIP | ESA1; TIP60; TIP; |
| | | | | | protein, 60 kDa | | PLIP; HTATIP1; |
| | | | | | (HTATIP), transcript | | cPLA2 |
| | | | | | variant 1, mRNA. | | |
| 113 | 70 | 1430347 | NM_001076785.1 | NM_001076785 | solute carrier family 7 | SLC7A6 | DKFZp686K15246; |
| | | | | | (cationic amino acid | | KIAA0245; LAT3; |
| | | | | | transporter, y+ system), | | LAT-2; y+LAT-2 |
| | | | | | member 6 (SLC7A6), | | |
| | | | | | transcript variant 2, | | |
| | | | | | mRNA. | | |
| 114 | 71 | 1440296 | NM_005324.3 | NM_005324 | H3 histone, family 3B | H3F3B | H3F3A; H3.3B |
| | | | | | (H3.3B) (H3F3B), | | |
| | | | | | mRNA. | | |
| 115 | 72 | 1440747 | NM_003544.2 | NM_003544 | histone cluster 1, H4b | HIST1H4B | H4FI; H4/I |
| | | | | | (HIST1H4B), mRNA. | | |
| 116 | 73 | 1470209 | NM_019026.2 | NM_019026 | transmembrane and | TMCO1 | HP10122; TMCC4; |
| | | | | | coiled-coil domains 1 | | RP11-466F5.7; |
| | | | | | (TMCO1), mRNA. | | PCIA3; PNAS-136 |
| 117 | 74 | 1510538 | NM_012307.2 | NM_012307 | erythrocyte membrane | EPB41L3 | DAL-1; KIAA0987; |
| | | | | | protein band 4.1-like 3 | | 4.1B; FLJ37633; |
| | | | | | (EPB41L3), mRNA. | | DAL1 |
| 118 | 75 | 1570575 | NM_014574.3 | NM_014574 | striatin, calmodulin | STRN3 | SG2NA |
| | | | | | binding protein 3 | | |
| | | | | | (STRN3), transcript | | |
| | | | | | variant 2, mRNA. | | |
| 119 | 76 | 1660687 | NM_001018089.1 | NM_001018089 | NMDA receptor | NARG2 | BRCC1 |
| | | | | | regulated 2 (NARG2), | | |
| | | | | | transcript variant 2, | | |
| | | | | | mRNA. | | |
| 120 | 77 | 1690189 | NM_152453.2 | NM_152453 | transmembrane and | TMCO5 | MGC35118; |
| | | | | | coiled-coil domains 5 | | FLJ35807 |
| | | | | | (TMCO5), mRNA. | | |
| 121 | 78 | 1740220 | NM_004038.3 | NM_004038 | amylase, alpha 1A | AMY1A | AMY1; AMY1B |
| | | | | | (salivary) (AMY1A), | | |
| | | | | | transcript variant 1, | | |
| | | | | | mRNA. | | |
| 122 | 79 | 1770609 | NM_198486.2 | NM_198486 | ribosomal protein L7- | RPL7L1 | MGC62004; |
| | | | | | like 1 (RPL7L1), | | dJ475N16.4 |
| | | | | | mRNA. | | |
| 123 | 80 | 1780273 | XM_001127464.1 | XM_001127464 | PREDICTED: | ALOX5 | — |
| | | | | | arachidonate 5- | | |
| | | | | | lipoxygenase (ALOX5), | | |
| | | | | | mRNA. | | |
| 124 | 81 | 1780647 | NM_052853.3 | NM_052853 | aarF domain containing | ADCK2 | MGC20727; AARF |
| | | | | | kinase 2 (ADCK2), | | |
| | | | | | mRNA. | | |
| 125 | 82 | 1820544 | NM_182679.1 | NM_182679 | G patch domain | GPATCH4 | GPATC4 |
| | | | | | containing 4 | | |
| | | | | | (GPATCH4), transcript | | |
| | | | | | variant 2, mRNA. | | |
| 126 | 83 | 1940041 | NM_000631.3 | NM_000631 | neutrophil cytosolic | NCF4 | SH3PXD4; |
| | | | | | factor 4, 40 kDa (NCF4), | | P40PHOX; NCF; |
| | | | | | transcript variant 1, | | MGC3810 |
| | | | | | mRNA. | | |
| 127 | 84 | 1940053 | NM_001681.2 | NM_001681 | ATPase, Ca++ | ATP2A2 | DAR; ATP2B; |
| | | | | | transporting, cardiac | | MGC45367; DD; |
| | | | | | muscle, slow twitch 2 | | SERCA2 |
| | | | | | (ATP2A2), transcript | | |
| | | | | | variant 2, mRNA. | | |
| 128 | 85 | 1980594 | NR_002203.1 | NR_002203 | ferritin, heavy | FTHL8 | — |
| | | | | | polypeptide-like 8 | | |
| | | | | | (FTHL8) on | | |
| | | | | | chromosome X. | | |
| 129 | 86 | 1990278 | NM_021642.2 | NM_021642 | Fc fragment of IgG, low | FCGR2A | FCGR2A1; CDw32; |
| | | | | | affinity IIa, receptor | | CD32A; CD32; FcGR; |
| | | | | | (CD32) (FCGR2A), | | FCG2; IGFR2; |
| | | | | | mRNA. | | FCGR2; MGC30032; |
| | | | | | | | MGC23887 |
| 130 | 87 | 2000010 | NM_006231.2 | NM_006231 | polymerase (DNA | POLE | DKFZp434F222; |
| | | | | | directed), epsilon | | FLJ21434; POLE1 |
| | | | | | (POLE), mRNA. | | |
| 131 | 88 | 2000048 | NM_173683.3 | NM_173683 | XK, Kell blood group | XKR6 | C8orf7; XRG6; |
| | | | | | complex subunit-related | | C8orf21 |
| | | | | | family, member 6 | | |
| | | | | | (XKR6), transcript | | |
| | | | | | variant 2, mRNA. | | |
| 132 | 89 | 2030243 | NM_013393.1 | NM_013393 | FtsJ homolog 2 (E. coli) | FTSJ2 | FJH1; |
| | | | | | (FTSJ2), mRNA. | | DKFZp686J14194 |
| 133 | 90 | 2060291 | NM_004099.4 | NM_004099 | stomatin (STOM), | STOM | EPB7; EPB72; BND7 |
| | | | | | transcript variant 1, | | |
| | | | | | mRNA. | | |
| 134 | 91 | 2070288 | NM_175617.3 | NM_175617 | metallothionein 1E | MT1E | MT1; MTD |
| | | | | | (MT1E), mRNA. | | |
| 135 | 92 | 2100196 | NM_005101.1 | NM_005101 | ISG15 ubiquitin-like | ISG15 | G1P2; UCRP; IFI15 |
| | | | | | modifier (ISG15), | | |
| | | | | | mRNA. | | |
| 136 | 93 | 2100273 | NM_001402.5 | NM_001402 | eukaryotic translation | EEF1A1 | EEF1A; FLJ25721; |
| | | | | | elongation factor 1 | | CCS-3; PTI1; CCS3; |
| | | | | | alpha 1 (EEF1A1), | | MGC102687; |
| | | | | | mRNA. | | MGC16224; EF-Tu; |
| | | | | | | | eEF1A-1; EEF-1; |
| | | | | | | | MGC131894; |
| | | | | | | | HNGC: 16303; GRAF- |
| | | | | | | | 1EF; LENG7; EF1A |
| 137 | 94 | 2100292 | NM_002893.2 | NM_002893 | retinoblastoma binding | RBBP7 | RbAp46; |
| | | | | | protein 7 (RBBP7), | | MGC138867; |
| | | | | | mRNA. | | MGC138868 |
| 138 | 95 | 2140753 | NM_001034996.1 | NM_001034996 | ribosomal protein L14 | RPL14 | CAG-ISL-7; CTG- |
| | | | | | (RPL14), transcript | | B33; L14; |
| | | | | | variant 1, mRNA. | | MGC88594; RL14; |
| | | | | | | | hRL14 |
| 139 | 96 | 2230678 | NM_001093.3 | NM_001093 | acetyl-Coenzyme A | ACACB | ACC2; ACCB; |
| | | | | | carboxylase beta | | HACC275 |
| | | | | | (ACACB), mRNA. | | |
| 140 | 97 | 2320053 | NM_024632.4 | NM_024632 | SAP30-like (SAP30L), | SAP30L | FLJ11526; NS4ATP2 |
| | | | | | mRNA. | | |
| 141 | 98 | 2320139 | NM_002954.3 | NM_002954 | ribosomal protein S27a | RPS27A | UBCEP1; UBA80; |
| | | | | | (RPS27A), mRNA. | | CEP80; HUBCEP80; |
| | | | | | | | UBCEP80 |
| 142 | 99 | 2320653 | NM_018281.2 | NM_018281 | enoyl Coenzyme A | ECHDC2 | FLJ10948 |
| | | | | | hydratase domain | | |
| | | | | | containing 2 | | |
| | | | | | (ECHDC2), mRNA. | | |
| 143 | 100 | 2340626 | NM_016020.1 | NM_016020 | transcription factor B1, | TFB1M | CGI75; mtTFB; CGI- |
| | | | | | mitochondrial (TFB1M), | | 75 |
| | | | | | mRNA. | | |
| 144 | 101 | 2350192 | NM_018694.2 | NM_018694 | ADP-ribosylation-like | ARL6IP4 | MGC814; SRp25; |
| | | | | | factor 6 interacting | | SR-25 |
| | | | | | protein 4 (ARL6IP4), | | |
| | | | | | transcript variant 1, | | |
| | | | | | mRNA. | | |
| 145 | 102 | 2350563 | NM_005791.1 | NM_005791 | M-phase | MPHOSPH10 | MPP10P; MPP10 |
| | | | | | phosphoprotein 10 (U3 | | |
| | | | | | small | | |
| | | | | | nucleolarribonucleoprotein) | | |
| | | | | | (MPHOSPH10), | | |
| | | | | | mRNA. | | |
| 146 | 103 | 2360528 | NM_182776.1 | NM_182776 | minichromosome | MCM7 | MCM2; CDC47; P1.1- |
| | | | | | maintenance complex | | MCM3; P1CDC47; |
| | | | | | component 7 (MCM7), | | CDABP0042; |
| | | | | | transcript variant 2, | | P85MCM; PNAS-146 |
| | | | | | mRNA. | | |
| 147 | 104 | 2450446 | NM_015906.3 | NM_015906 | tripartite motif- | TRIM33 | FLJ32925; TIF1G; |
| | | | | | containing 33 | | RFG7; PTC7; TF1G; |
| | | | | | (TRIM33), transcript | | TIF1GAMMA; |
| | | | | | variant a, mRNA. | | TIFGAMMA |
| 148 | 105 | 2480037 | NM_178868.3 | NM_178868 | CKLF-like MARVEL | CMTM8 | CKLFSF8; CKLFSF8- |
| | | | | | transmembrane | | V2 |
| | | | | | domain containing 8 | | |
| | | | | | (CMTM8), mRNA. | | |
| 149 | 106 | 2480328 | NM_032361.1 | NM_032361 | THO complex 3 | THOC3 | TEX1; MGC5469 |
| | | | | | (THOC3), mRNA. | | |
| 150 | 107 | 2480487 | NM_005819.4 | NM_005819 | syntaxin 6 (STX6), | STX6 | — |
| | | | | | mRNA. | | |
| 151 | 108 | 2490333 | NM_207336.1 | NM_207336 | zinc finger protein 467 | ZNF467 | EZI; Zfp467 |
| | | | | | (ZNF467), mRNA. | | |
| 152 | 109 | 2570100 | NM_019112.3 | NM_019112 | ATP-binding cassette, | ABCA7 | ABCA-SSN; ABCX; |
| | | | | | sub-family A (ABC1), | | FLJ40025 |
| | | | | | member 7 (ABCA7), | | |
| | | | | | mRNA. | | |
| 153 | 110 | 2570288 | NM_015677.1 | NM_015677 | SH3 domain | SH3YL1 | FLJ39121; Ray; |
| | | | | | containing, Ysc84-like 1 | | DKFZP586F1318 |
| | | | | | (S. cerevisiae) | | |
| | | | | | (SH3YL1), mRNA. | | |
| 154 | 111 | 2570328 | NM_021643.1 | NM_021643 | tribbles homolog 2 | TRIB2 | TRB2; GS3955 |
| | | | | | (Drosophila) (TRIB2), | | |
| | | | | | mRNA. | | |
| 155 | 112 | 2600204 | NM_014016.2 | NM_014016 | SAC1 suppressor of | SACM1L | KIAA0851; SAC1; |
| | | | | | actin mutations 1-like | | DKFZp686A0231 |
| | | | | | (yeast) (SACM1L), | | |
| | | | | | mRNA. | | |
| 156 | 113 | 2640541 | NM_006364.2 | NM_006364 | Sec23 homolog A (S. cerevisiae) | SEC23A | CLSD; MGC26267 |
| | | | | | (SEC23A), | | |
| | | | | | mRNA. | | |
| 157 | 114 | 2640707 | XM_001130839.1 | XM_001130839 | PREDICTED: nuclear | NR1D2 | — |
| | | | | | receptor subfamily 1, | | |
| | | | | | group D, member 2 | | |
| | | | | | (NR1D2), mRNA. | | |
| 158 | 115 | 2680082 | NM_000971.3 | NM_000971 | ribosomal protein L7 | RPL7 | MGC117326; humL7-1 |
| | | | | | (RPL7), mRNA. | | |
| 159 | 116 | 2690224 | NM_030980.1 | NM_030980 | interferon stimulated | ISG20L2 | FLJ12671 |
| | | | | | exonuclease gene | | |
| | | | | | 20 kDa-like 2 | | |
| | | | | | (ISG20L2), mRNA. | | |
| 160 | 117 | 2710196 | NM_018428.2 | NM_018428 | UTP6, small subunit | UTP6 | HCA66; C17orf40 |
| | | | | | (SSU) processome | | |
| | | | | | component, homolog | | |
| | | | | | (yeast) (UTP6), mRNA. | | |
| 161 | 118 | 2760537 | NM_175621.2 | NM_175621 | metallothionein E | MTE | MT1I |
| | | | | | (MTE), mRNA. | | |
| 162 | 119 | 2970079 | NM_003274.3 | NM_003274 | transmembrane protein | TMEM1 | EHOC-1; |
| | | | | | 1 (TMEM1), transcript | | MGC126777; |
| | | | | | variant 1, mRNA. | | EHOC1; GT334 |
| 163 | 120 | 2970594 | NM_138373.3 | NM_138373 | myeloid-associated | MYADM | SB135 |
| | | | | | differentiation marker | | |
| | | | | | (MYADM), transcript | | |
| | | | | | variant 2, mRNA. | | |
| 164 | 121 | 3130600 | NM_007048.4 | NM_007048 | butyrophilin, subfamily | BTN3A1 | BT3.1; CD277; |
| | | | | | 3, member A1 | | MGC141880; BTF5 |
| | | | | | (BTN3A1), mRNA. | | |
| 165 | 122 | 3140041 | NM_007237.3 | NM_007237 | SP140 nuclear body | SP140 | MGC126440; |
| | | | | | protein (SP140), | | LYSP100-B; |
| | | | | | transcript variant 1, | | LYSP100-A |
| | | | | | mRNA. | | |
| 166 | 123 | 3170440 | NM_022893.2 | NM_022893 | B-cell CLL/lymphoma | BCL11A | BCL11A-L; CTIP1; |
| | | | | | 11A (zinc finger protein) | | FLJ10173; EVI9; |
| | | | | | (BCL11A), transcript | | BCL11A-XL; |
| | | | | | variant 1, mRNA. | | BCL11A-S; |
| | | | | | | | FLJ34997; KIAA1809 |
| 167 | 124 | 3170451 | NM_024815.3 | NM_024815 | nudix (nucleoside | NUDT18 | FLJ22494 |
| | | | | | diphosphate linked | | |
| | | | | | moiety X)-type motif 18 | | |
| | | | | | (NUDT18), mRNA. | | |
| 168 | 125 | 3180273 | NM_020315.4 | NM_020315 | pyridoxal (pyridoxine, | PDXP | CIN; FLJ32703; PLP; |
| | | | | | vitamin B6) | | dJ37E16.5 |
| | | | | | phosphatase (PDXP), | | |
| | | | | | mRNA. | | |
| 169 | 126 | 3190133 | NR_002205.1 | NR_002205 | ferritin, heavy | FTHL12 | — |
| | | | | | polypeptide-like 12 | | |
| | | | | | (FTHL12) on | | |
| | | | | | chromosome 9. | | |
| 170 | 127 | 3310546 | NM_001950.3 | NM_001950 | E2F transcription factor | E2F4 | E2F-4 |
| | | | | | 4, p107/p130-binding | | |
| | | | | | (E2F4), mRNA. | | |
| 171 | 128 | 3370474 | NM_013368.2 | NM_013368 | SERTA domain | SERTAD3 | RBT1 |
| | | | | | containing 3 | | |
| | | | | | (SERTAD3), transcript | | |
| | | | | | variant 1, mRNA. | | |
| 172 | 129 | 3450278 | NM_172232.1 | NM_172232 | ATP-binding cassette, | ABCA5 | FLJ16381; |
| | | | | | sub-family A (ABC1), | | DKFZp779N2435; |
| | | | | | member 5 (ABCA5), | | DKFZp451F117; |
| | | | | | transcript variant 2, | | EST90625; ABC13 |
| | | | | | mRNA. | | |
| 173 | 130 | 3450463 | NM_183376.1 | NM_183376 | arrestin domain | ARRDC4 | FLJ36045 |
| | | | | | containing 4 | | |
| | | | | | (ARRDC4), mRNA. | | |
| 174 | 131 | 3450537 | NM_032564.2 | NM_032564 | diacylglycerol O- | DGAT2 | HMFN1045; |
| | | | | | acyltransferase | | DKFZp686A15125 |
| | | | | | homolog 2 (mouse) | | |
| | | | | | (DGAT2), mRNA. | | |
| 175 | 132 | 3520093 | NM_021070.2 | NM_021070 | latent transforming | LTBP3 | FLJ44138; FLJ42533; |
| | | | | | growth factor beta | | FLJ39893; LTBP-3; |
| | | | | | binding protein 3 | | pp6425; FLJ33431; |
| | | | | | (LTBP3), mRNA. | | LTBP2; |
| | | | | | | | DKFZP586M2123 |
| 176 | 133 | 3520598 | NM_019858.1 | NM_019858 | G protein-coupled | GPR162 | GRCA; A-2 |
| | | | | | receptor 162 | | |
| | | | | | (GPR162), transcript | | |
| | | | | | variant A-2, mRNA. | | |
| 177 | 134 | 3610630 | NM_016302.2 | NM_016302 | cereblon (CRBN), | CRBN | MGC27358; |
| | | | | | mRNA. | | DKFZp781K0715; |
| | | | | | | | MRT2A |
| 178 | 135 | 3710735 | NM_153819.1 | NM_153819 | RAS guanyl releasing | RASGRP2 | CDC25L; CALDAG- |
| | | | | | protein 2 (calcium and | | GEFI |
| | | | | | DAG-regulated) | | |
| | | | | | (RASGRP2), transcript | | |
| | | | | | variant 2, mRNA. | | |
| 179 | 136 | 3780544 | NM_016047.3 | NM_016047 | splicing factor 3B, 14 kDa | SF3B14 | Ht006; SF3B14a; |
| | | | | | subunit (SF3B14), | | SAP14; CGI-110; |
| | | | | | mRNA. | | HSPC175; P14 |
| 180 | 137 | 3800576 | NM_080914.1 | NM_080914 | asialoglycoprotein | ASGR2 | L-H2; CLEC4H2; |
| | | | | | receptor 2 (ASGR2), | | Hs.1259; ASGP-R |
| | | | | | transcript variant 3, | | |
| | | | | | mRNA. | | |
| 181 | 138 | 3830273 | NM_020202.2 | NM_020202 | nitrilase family, member | NIT2 | MGC111199 |
| | | | | | 2 (NIT2), mRNA. | | |
| 182 | 139 | 3830653 | NM_006736.5 | NM_006736 | DnaJ (Hsp40) homolog, | DNAJB2 | HSPF3; HSJ1 |
| | | | | | subfamily B, member 2 | | |
| | | | | | (DNAJB2), transcript | | |
| | | | | | variant 2, mRNA. | | |
| 183 | 140 | 3850059 | NM_005574.2 | NM_005574 | LIM domain only 2 | LMO2 | TTG2; RBTN2; |
| | | | | | (rhombotin-like 1) | | RBTNL1; RHOM2 |
| | | | | | (LMO2), mRNA. | | |
| 184 | 141 | 3890689 | NM_198053.1 | NM_198053 | CD247 molecule | CD247 | CD3Q; CD3H; TCRZ; |
| | | | | | (CD247), transcript | | CD3Z; CD3-ZETA |
| | | | | | variant 1, mRNA. | | |
| 185 | 142 | 3930133 | NM_199004.1 | NM_199004 | arrestin, beta 2 | ARRB2 | ARR2; ARB2; |
| | | | | | (ARRB2), transcript | | DKFZp686L0365 |
| | | | | | variant 2, mRNA. | | |
| 186 | 143 | 3930392 | NM_001097577.1 | NM_001097577 | angiogenin, | ANG | RNASE5; |
| | | | | | ribonuclease, RNase A | | MGC22466; |
| | | | | | family, 5 (ANG), | | RNASE4; MGC71966 |
| | | | | | transcript variant 2, | | |
| | | | | | mRNA. | | |
| 187 | 144 | 3940138 | NM_001009944.1 | NM_001009944 | polycystic kidney | PKD1 | PBP |
| | | | | | disease 1 (autosomal | | |
| | | | | | dominant) (PKD1), | | |
| | | | | | transcript variant 1, | | |
| | | | | | mRNA. | | |
| 188 | 145 | 3940358 | NM_001003712.1 | NM_001003712 | oxysterol binding | OSBPL8 | MSTP120; ORP8; |
| | | | | | protein-like 8 | | MST120; |
| | | | | | (OSBPL8), transcript | | MGC126578; |
| | | | | | variant 2, mRNA. | | DKFZp686A11164; |
| | | | | | | | OSBP10; |
| | | | | | | | MGC133203 |
| 189 | 146 | 3990112 | NM_001042445.1 | NM_001042445 | calpastatin (CAST), | CAST | MGC9402; BS-17 |
| | | | | | transcript variant 11, | | |
| | | | | | mRNA. | | |
| 190 | 147 | 4010400 | NM_002480.1 | NM_002480 | protein phosphatase 1, | PPP1R12A | MGC133042; MYPT1; |
| | | | | | regulatory (inhibitor) | | MBS |
| | | | | | subunit 12A | | |
| | | | | | (PPP1R12A), mRNA. | | |
| 191 | 148 | 4040088 | NM_152772.1 | NM_152772 | t-complex 11 (mouse)- | TCP11L2 | MGC40368 |
| | | | | | like 2 (TCP11L2), | | |
| | | | | | mRNA. | | |
| 192 | 149 | 4120039 | NR_002200.1 | NR_002200 | ferritin, heavy | FTHL2 | — |
| | | | | | polypeptide-like 2 | | |
| | | | | | (FTHL2) on | | |
| | | | | | chromosome 1. | | |
| 193 | 150 | 4120341 | NM_002208.4 | NM_002208 | integrin, alpha E | ITGAE | HUMINAE; CD103; |
| | | | | | (antigen CD103, | | MGC141996 |
| | | | | | human mucosal | | |
| | | | | | lymphocyte antigen 1; | | |
| | | | | | alpha polypeptide) | | |
| | | | | | (ITGAE), mRNA. | | |
| 194 | 151 | 4150132 | NM_017514.2 | NM_017514 | plexin A3 (PLXNA3), | PLXNA3 | XAP-6; |
| | | | | | mRNA. | | HSSEXGENE; |
| | | | | | | | PLEXIN-A3; PLXN4; |
| | | | | | | | SEX; PLXN3; 6.3 |
| 195 | 152 | 4200068 | NM_016553.3 | NM_016553 | nucleoporin 62 kDa | NUP62 | FLJ43869; |
| | | | | | (NUP62), transcript | | DKFZp547L134; |
| | | | | | variant 2, mRNA. | | MGC841; p62; SNDI; |
| | | | | | | | IBSN; FLJ20822 |
| 196 | 153 | 4210465 | NM_006889.3 | NM_006889 | CD86 molecule | CD86 | B7-2; B70; LAB72; |
| | | | | | (CD86), transcript | | MGC34413; |
| | | | | | variant 2, mRNA. | | CD28LG2 |
| 197 | 154 | 4220468 | NM_001001787.1 | NM_001001787 | ATPase, Na+/K+ | ATP1B1 | MGC1798; ATP1B |
| | | | | | transporting, beta 1 | | |
| | | | | | polypeptide (ATP1B1), | | |
| | | | | | transcript variant 2, | | |
| | | | | | mRNA. | | |
| 198 | 155 | 4220632 | XM_001133534.1 | XM_001133534 | PREDICTED: ATPase, | ATP1B3 | — |
| | | | | | Na+/K+ transporting, | | |
| | | | | | beta 3 polypeptide, | | |
| | | | | | transcript variant 2 | | |
| | | | | | (ATP1B3), mRNA. | | |
| 199 | 156 | 4220672 | NM_005949.2 | NM_005949 | metallothionein 1F | MT1F | MT1; MGC32732 |
| | | | | | (MT1F), mRNA. | | |
| 200 | 157 | 4220731 | NM_000917.2 | NM_000917 | procollagen-proline, 2- | P4HA1 | P4HA; 4-PH alpha-1 |
| | | | | | oxoglutarate 4- | | |
| | | | | | dioxygenase (proline 4- | | |
| | | | | | hydroxylase), alpha | | |
| | | | | | polypeptide I (P4HA1), | | |
| | | | | | transcript variant 1, | | |
| | | | | | mRNA. | | |
| 201 | 158 | 4230093 | NM_001171.3 | NM_001171 | ATP-binding cassette, | ABCC6 | MOATE; EST349056; |
| | | | | | sub-family C | | ARA; PXE1; ABC34; |
| | | | | | (CFTR/MRP), member | | PXE; MLP1; MRP6 |
| | | | | | 6 (ABCC6), transcript | | |
| | | | | | variant 1, mRNA. | | |
| 202 | 159 | 4230097 | NM_002128.4 | NM_002128 | high-mobility group box | HMGB1 | DKFZp686A04236; |
| | | | | | 1 (HMGB1), mRNA. | | HMG3; SBP-1; HMG1 |
| 203 | 161 | 4250768 | NM_004645.2 | NM_004645 | coilin (COIL), mRNA. | COIL | p80-coilin; CLN80 |
| 204 | 162 | 4260221 | NM_145911.1 | NM_145911 | zinc finger protein 23 | ZNF23 | KOX16; Zfp612; |
| | | | | | (KOX 16) (ZNF23), | | ZNF359; ZNF612 |
| | | | | | mRNA. | | |
| 205 | 163 | 4280162 | NM_024041.2 | NM_024041 | sodium channel | SCNM1 | MGC3180 |
| | | | | | modifier 1 (SCNM1), | | |
| | | | | | mRNA. | | |
| 206 | 164 | 4390301 | NM_016113.3 | NM_016113 | transient receptor | TRPV2 | VRL; VRL-1; |
| | | | | | potential cation | | MGC12549; VRL1 |
| | | | | | channel, subfamily V, | | |
| | | | | | member 2 (TRPV2), | | |
| | | | | | mRNA. | | |
| 207 | 165 | 4490242 | NM_006256.2 | NM_006256 | protein kinase N2 | PKN2 | PRO2042; PAK2; |
| | | | | | (PKN2), mRNA. | | Pak-2; PRKCL2; |
| | | | | | | | MGC150606; |
| | | | | | | | MGC71074; PRK2 |
| 208 | 166 | 4640220 | NM_145113.1 | NM_145113 | MYC associated factor | MAX | MGC34679; |
| | | | | | X (MAX), transcript | | MGC36767; |
| | | | | | variant 3, mRNA. | | MGC11225; |
| | | | | | | | MGC10775; orf1; |
| | | | | | | | MGC18164 |
| 209 | 167 | 4670601 | NM_022804.2 | NM_022804 | SNRPN upstream | SNURF | — |
| | | | | | reading frame | | |
| | | | | | (SNURF), transcript | | |
| | | | | | variant 2, mRNA. | | |
| 210 | 168 | 4730148 | NM_004986.2 | NM_004986 | kinectin 1 (kinesin | KTN1 | KIAA0004; |
| | | | | | receptor) (KTN1), | | MGC133337; MU- |
| | | | | | transcript variant 4, | | RMS-40.19; CG1; |
| | | | | | mRNA. | | KNT |
| 211 | 169 | 4730181 | NR_002205.1 | NR_002205 | ferritin, heavy | FTHL12 | — |
| | | | | | polypeptide-like 12 | | |
| | | | | | (FTHL12) on | | |
| | | | | | chromosome 9. | | |
| 212 | 170 | 4760474 | NM_006000.1 | NM_006000 | tubulin, alpha 4a | TUBA4A | TUBA1; H2-ALPHA; |
| | | | | | (TUBA4A), mRNA. | | FLJ30169 |
| 213 | 171 | 4780678 | NM_001079.3 | NM_001079 | zeta-chain (TCR) | ZAP70 | FLJ17670; ZAP-70; |
| | | | | | associated protein | | TZK; STD; FLJ17679; |
| | | | | | kinase 70 kDa (ZAP70), | | SRK |
| | | | | | transcript variant 1, | | |
| | | | | | mRNA. | | |
| 214 | 173 | 4850091 | NM_006331.5 | NM_006331 | EMG1 nucleolar protein | EMG1 | Grcc2f; C2F; NEP1 |
| | | | | | homolog (S. cerevisiae) | | |
| | | | | | (EMG1), mRNA. | | |
| 215 | 174 | 4850327 | NM_016205.1 | NM_016205 | platelet derived growth | PDGFC | SCDGF |
| | | | | | factor C (PDGFC), | | |
| | | | | | mRNA. | | |
| 216 | 175 | 4860209 | NM_173468.2 | NM_173468 | MOB1, Mps One | MOBKL1A | MOB4A; MGC33910; |
| | | | | | Binder kinase activator- | | MATS2; Mob1B |
| | | | | | like 1A (yeast) | | |
| | | | | | (MOBKL1A), mRNA. | | |
| 217 | 176 | 4880215 | NM_001514.3 | NM_001514 | general transcription | GTF2B | TFIIB; TF2B |
| | | | | | factor IIB (GTF2B), | | |
| | | | | | mRNA. | | |
| 218 | 177 | 4890722 | NM_006139.1 | NM_006139 | CD28 molecule | CD28 | Tp44; MGC138290 |
| | | | | | (CD28), mRNA. | | |
| 219 | 178 | 4920347 | NM_016442.3 | NM_016442 | endoplasmic reticulum | ERAP1 | APPILS; ALAP; |
| | | | | | aminopeptidase 1 | | PILSAP; ERAP1; |
| | | | | | (ERAP1), transcript | | ERAAP; ARTS-1; |
| | | | | | variant 1, mRNA. | | ERAAP1; KIAA0525; |
| | | | | | | | A-LAP; PILS-AP; |
| | | | | | | | ARTS1 |
| 220 | 179 | 5050156 | NM_004050.2 | NM_004050 | BCL2-like 2 (BCL2L2), | BCL2L2 | KIAA0271; BCLW; |
| | | | | | mRNA. | | BCL-W |
| 221 | 180 | 5080246 | NM_003522.3 | NM_003522 | histone cluster 1, H2bf | HIST1H2BF | H2B/g; H2BFG |
| | | | | | (HIST1H2BF), mRNA. | | |
| 222 | 182 | 5090307 | NM_153362.1 | NM_153362 | protease, serine, 35 | PRSS35 | dJ223E3.1; |
| | | | | | (PRSS35), mRNA. | | MGC46520; C6orf158 |
| 223 | 183 | 5090397 | NM_206909.2 | NM_206909 | pleckstrin and Sec7 | PSD3 | DKFZp761K1423; |
| | | | | | domain containing 3 | | EFA6R; HCA67 |
| | | | | | (PSD3), transcript | | |
| | | | | | variant 2, mRNA. | | |
| 224 | 184 | 5090450 | NM_004818.2 | NM_004818 | DEAD (Asp-Glu-Ala- | DDX23 | U5-100K; prp28; |
| | | | | | Asp) box polypeptide | | PRPF28; MGC8416 |
| | | | | | 23 (DDX23), mRNA. | | |
| 225 | 185 | 5130750 | NM_002729.4 | NM_002729 | hematopoietically | HHEX | HEX; PRH; PRHX; |
| | | | | | expressed homeobox | | HOX11L-PEN; HMPH |
| | | | | | (HHEX), mRNA. | | |
| 226 | 186 | 5270291 | NM_017811.3 | NM_017811 | ubiquitin-conjugating | UBE2R2 | UBC3B; FLJ20419; |
| | | | | | enzyme E2R 2 | | MGC10481; CDC34B |
| | | | | | (UBE2R2), mRNA. | | |
| 227 | 187 | 5290369 | NM_032582.3 | NM_032582 | ubiquitin specific | USP32 | USP10; NY-REN-60 |
| | | | | | peptidase 32 (USP32), | | |
| | | | | | mRNA. | | |
| 228 | 188 | 5290482 | NM_031943.1 | NM_031943 | IFP38 (IFP38), mRNA. | IFP38 | — |
| 229 | 189 | 5360500 | NM_152246.1 | NM_152246 | carnitinepalmitoyltransferase | CPT1B | CPT1-M; KIAA1670; |
| | | | | | 1B (muscle) | | M-CPT1 |
| | | | | | (CPT1B), nuclear gene | | |
| | | | | | encoding mitochondrial | | |
| | | | | | protein, transcript | | |
| | | | | | variant 3, mRNA. | | |
| 230 | 190 | 5390433 | NM_030621.2 | NM_030621 | Dicer1, Dcr-1 homolog | DICER1 | Dicer; HERNA; |
| | | | | | (Drosophila) (DICER1), | | KIAA0928 |
| | | | | | transcript variant 2, | | |
| | | | | | mRNA. | | |
| 231 | 191 | 5420575 | NM_001013251.1 | NM_001013251 | solute carrier family 3 | SLC3A2 | 4F2HC; CD98; 4F2; |
| | | | | | (activators of dibasic | | CD98HC; 4T2HC; |
| | | | | | and neutral amino acid | | MDU1; NACAE |
| | | | | | transport), member 2 | | |
| | | | | | (SLC3A2), transcript | | |
| | | | | | variant 6, mRNA. | | |
| 232 | 192 | 5490753 | NM_005467.2 | NM_005467 | N-acetylated alpha- | NAALAD2 | MGC26353; |
| | | | | | linked acidic | | MGC116996; |
| | | | | | dipeptidase 2 | | NAALADASE2; |
| | | | | | (NAALAD2), mRNA. | | NAADALASE2 |
| 233 | 193 | 5550369 | NM_001125.2 | NM_001125 | ADP-ribosylarginine | ADPRH | ARH1 |
| | | | | | hydrolase (ADPRH), | | |
| | | | | | mRNA. | | |
| 234 | 194 | 5670398 | NM_025191.2 | NM_025191 | ER degradation | EDEM3 | C1orf22 |
| | | | | | enhancer, | | |
| | | | | | mannosidase alpha-like | | |
| | | | | | 3 (EDEM3), mRNA. | | |
| 235 | 195 | 5670682 | XM_943640.2 | XM_943640 | PREDICTED: | FLJ32255 | — |
| | | | | | hypothetical protein | | |
| | | | | | LOC643977, transcript | | |
| | | | | | variant 2 (FLJ32255), | | |
| | | | | | mRNA. | | |
| 236 | 196 | 5810398 | NM_001080547.1 | NM_001080547 | spleen focus forming | SPI1 | SPI-A; OF; SFPI1; |
| | | | | | virus (SFFV) | | PU.1; SPI-1 |
| | | | | | proviralintegration | | |
| | | | | | oncogene spi1 (SPI1), | | |
| | | | | | transcript variant 1, | | |
| | | | | | mRNA. | | |
| 237 | 197 | 5820068 | NM_032025.3 | NM_032025 | eukaryotic translation | EIF2A | MSTP089; MSTP004; |
| | | | | | initiation factor 2A, | | EIF-2A; CDA02; |
| | | | | | 65 kDa (EIF2A), mRNA. | | MST089 |
| 238 | 198 | 5820528 | NM_001077446.1 | NM_001077446 | tRNA splicing | TSEN34 | LENG5; SEN34; |
| | | | | | endonuclease 34 | | SEN34L |
| | | | | | homolog (S. cerevisiae) | | |
| | | | | | (TSEN34), transcript | | |
| | | | | | variant 2, mRNA. | | |
| 239 | 199 | 5860064 | NM_138782.1 | NM_138782 | FCH domain only 2 | FCHO2 | — |
| | | | | | (FCHO2), mRNA. | | |
| 240 | 200 | 5870131 | NM_000492.3 | NM_000492 | cystic fibrosis | CFTR | ABCC7; MRP7; TNR- |
| | | | | | transmembrane | | CFTR; CFTR/MRP; |
| | | | | | conductance regulator | | dJ760C5.1; CF; |
| | | | | | (ATP-binding cassette | | ABC35 |
| | | | | | sub-family C, member | | |
| | | | | | 7) (CFTR), mRNA. | | |
| 241 | 201 | 5890538 | NM_018708.2 | NM_018708 | fem-1 homolog a (C. elegans) | FEM1A | DKFZp762M136; |
| | | | | | (FEM1A), | | EPRAP |
| | | | | | mRNA. | | |
| 242 | 202 | 5900112 | NM_052857.2 | NM_052857 | coiled-coil domain | CCDC16 | MGC20398 |
| | | | | | containing 16 | | |
| | | | | | (CCDC16), mRNA. | | |
| 243 | 203 | 5910113 | NM_004385.2 | NM_004385 | versican (VCAN), | VCAN | DKFZp686K06110; |
| | | | | | mRNA. | | WGN; VERSICAN; |
| | | | | | | | PG-M; WGN1; ERVR; |
| | | | | | | | CSPG2 |
| 244 | 204 | 6020327 | NM_024901.3 | NM_024901 | DENN/MADD domain | DENND2D | FLJ22457; RP5- |
| | | | | | containing 2D | | 1180E21.2 |
| | | | | | (DENND2D), mRNA. | | |
| 245 | 205 | 6020653 | NM_014962.2 | NM_014962 | BTB (POZ) domain | BTBD3 | dJ742J24.1; |
| | | | | | containing 3 (BTBD3), | | MGC130038; |
| | | | | | transcript variant 1, | | KIAA0952; |
| | | | | | mRNA. | | MGC130039 |
| 246 | 206 | 6040487 | NM_006265.1 | NM_006265 | RAD21 homolog (S. pombe) | RAD21 | KIAA0078; hHR21; |
| | | | | | (RAD21), | | NXP1; FLJ40596; |
| | | | | | mRNA. | | HRAD21; FLJ25655; |
| | | | | | | | SCC1; HR21; MCD1 |
| 247 | 208 | 6110392 | NM_002076.2 | NM_002076 | glucosamine (N-acetyl)- | GNS | G6S; MGC21274 |
| | | | | | 6-sulfatase (Sanfilippo | | |
| | | | | | disease IIID) (GNS), | | |
| | | | | | mRNA. | | |
| 248 | 209 | 6180070 | NR_002204.1 | NR_002204 | ferritin, heavy | FTHL11 | — |
| | | | | | polypeptide-like 11 | | |
| | | | | | (FTHL11) on | | |
| | | | | | chromosome 8. | | |
| 249 | 210 | 6180154 | NM_145255.2 | NM_145255 | mitochondrial ribosomal | MRPL10 | MGC17973; MRP- |
| | | | | | protein L10 (MRPL10), | | L10; L10MT; RPML8; |
| | | | | | nuclear gene encoding | | MRPL8; MRP-L8 |
| | | | | | mitochondrial protein, | | |
| | | | | | mRNA. | | |
| 250 | 211 | 6180537 | NM_002139.2 | NM_002139 | RNA binding motif | RBMX | RBMXRT; HNRPG; |
| | | | | | protein, X-linked | | hnRNP-G; RNMX; |
| | | | | | (RBMX), mRNA. | | RBMXP1 |
| 251 | 212 | 6200402 | NM_005946.2 | NM_005946 | metallothionein 1A | MT1A | MTC; MT1; |
| | | | | | (MT1A), mRNA. | | MGC32848; MT1S |
| 252 | 213 | 6200669 | NM_138720.1 | NM_138720 | histone cluster 1, H2bd | HIST1H2BD | H2B.1B; HIRIP2; |
| | | | | | (HIST1H2BD), | | MGC90432; |
| | | | | | transcript variant 2, | | dJ221C16.6; H2B/b; |
| | | | | | mRNA. | | H2BFB |
| 253 | 214 | 6290402 | NM_198723.1 | NM_198723 | transcription elongation | TCEA2 | TFIIS |
| | | | | | factor A (SII), 2 | | |
| | | | | | (TCEA2), transcript | | |
| | | | | | variant 2, mRNA. | | |
| 254 | 215 | 6370025 | NM_013333.2 | NM_013333 | epsin 1 (EPN1), mRNA. | EPN1 | — |
| 255 | 216 | 6370241 | NM_014155.3 | NM_014155 | zinc finger and BTB | ZBTB44 | MGC57431; |
| | | | | | domain containing 44 | | MGC60348; BTBD15; |
| | | | | | (ZBTB44), mRNA. | | MGC88058; |
| | | | | | | | HSPC063; |
| | | | | | | | MGC26123 |
| 256 | 217 | 6380347 | NM_001469.3 | NM_001469 | X-ray repair | XRCC6 | TLAA; G22P1; |
| | | | | | complementing | | CTCBF; ML8; CTC75; |
| | | | | | defective repair in | | KU70 |
| | | | | | Chinese hamster cells | | |
| | | | | | 6 (Ku autoantigen, | | |
| | | | | | 70 kDa) (XRCC6), | | |
| | | | | | mRNA. | | |
| 257 | 218 | 6380524 | NM_003432.1 | NM_003432 | zinc finger protein 131 | ZNF131 | pHZ-10 |
| | | | | | (ZNF131), mRNA. | | |
| 258 | 219 | 6380639 | NM_213725.1 | NM_213725 | ribosomal protein, | RPLP1 | P1; FLJ27448; RPP1; |
| | | | | | large, P1 (RPLP1), | | MGC5215 |
| | | | | | transcript variant 2, | | |
| | | | | | mRNA. | | |
| 259 | 221 | 6400332 | NM_014184.2 | NM_014184 | cornichon homolog 4 | CNIH4 | HSPC163 |
| | | | | | (Drosophila) (CNIH4), | | |
| | | | | | mRNA. | | |
| 260 | 222 | 6400603 | NM_024070.3 | NM_024070 | poliovirus receptor | PVRIG | MGC138297; |
| | | | | | related immunoglobulin | | MGC2463; |
| | | | | | domain containing | | MGC138295; |
| | | | | | (PVRIG), mRNA. | | MGC104322; C7orf15 |
| 261 | 223 | 6420730 | NM_001024921.2 | NM_001024921 | ribosomal protein L9 | RPL9 | FLJ27456; |
| | | | | | (RPL9), transcript | | MGC15545; |
| | | | | | variant 2, mRNA. | | DKFZp313J1510; |
| | | | | | | | NPC-A-16 |
| 262 | 225 | 6550315 | NM_020424.2 | NM_020424 | LYR motif containing 1 | LYRM1 | A211C6.1 |
| | | | | | (LYRM1), mRNA. | | |
| 263 | 226 | 6560121 | NM_002647.2 | NM_002647 | phosphoinositide-3- | PIK3C3 | MGC61518; Vps34 |
| | | | | | kinase, class 3 | | |
| | | | | | (PIK3C3), mRNA. | | |
| 264 | 227 | 6560164 | NM_001006.3 | NM_001006 | ribosomal protein S3A | RPS3A | FTE1; MGC23240; |
| | | | | | (RPS3A), mRNA. | | MFTL |
| 265 | 228 | 6580121 | NM_021242.3 | NM_021242 | MID1 interacting protein | MID1IP1 | THRSPL; MIG12; |
| | | | | | 1 (gastrulation specific | | STRAIT11499; |
| | | | | | G12 homolog | | FLJ10386; G12-like |
| | | | | | (zebrafish)) (MID1IP1), | | |
| | | | | | mRNA. | | |
| 266 | 229 | 6620528 | NM_005952.2 | NM_005952 | metallothionein 1X | MT1X | MT1; MT-1I |
| | | | | | (MT1X), mRNA. | | |
| 267 | 230 | 6620544 | NM_016360.2 | NM_016360 | coiled-coil domain | CCDC44 | — |
| | | | | | containing 44 | | |
| | | | | | (CCDC44), mRNA. | | |
| 268 | 232 | 6660162 | NM_052972.2 | NM_052972 | leucine-rich alpha-2- | LRG1 | HMFT1766; LRG |
| | | | | | glycoprotein 1 (LRG1), | | |
| | | | | | mRNA. | | |
| 269 | 233 | 6760192 | NM_007236.3 | NM_007236 | calcium binding protein | CHP | SLC9A1BP |
| | | | | | P22 (CHP), mRNA. | | |
| 270 | 234 | 6770634 | NM_005154.2 | NM_005154 | ubiquitin specific | USP8 | KIAA0055; FLJ34456; |
| | | | | | peptidase 8 (USP8), | | MGC129718; UBPY; |
| | | | | | mRNA. | | HumORF8 |
| 271 | 235 | 6840020 | NM_006573.3 | NM_006573 | tumor necrosis factor | TNFSF13B | TNFSF20; CD257; |
| | | | | | (ligand) superfamily, | | TALL1; delta BAFF; |
| | | | | | member 13b | | BAFF; ZTNF4; TALL- |
| | | | | | (TNFSF13B), mRNA. | | 1; THANK; BLYS |
| 272 | 236 | 6900528 | NM_001033568.1 | NM_001033568 | ras homolog gene | RHOT1 | ARHT1; MIRO-1; |
| | | | | | family, member T1 | | FLJ12633; FLJ11040 |
| | | | | | (RHOT1), transcript | | |
| | | | | | variant 1, mRNA. | | |
| 273 | 238 | 6960735 | NM_006004.1 | NM_006004 | ubiquinol-cytochrome c | UQCRH | — |
| | | | | | reductase hinge protein | | |
| | | | | | (UQCRH), mRNA. | | |
| 274 | 239 | 6980092 | NM_024297.2 | NM_024297 | PHD finger protein 23 | PHF23 | hJUNE-1b; |
| | | | | | (PHF23), mRNA. | | MGC2941; FLJ16355; |
| | | | | | | | FLJ22884 |
| 275 | 240 | 7000369 | NM_000591.2 | NM_000591 | CD14 molecule | CD14 | — |
| | | | | | (CD14), transcript | | |
| | | | | | variant 1, mRNA. | | |
| 276 | 241 | 7000465 | NM_153615.1 | NM_153615 | ral guanine nucleotide | RGL4 | Rgr, MGC119678; |
| | | | | | dissociation stimulator- | | MGC119680 |
| | | | | | like 4 (RGL4), mRNA. | | |
| 277 | 242 | 7050670 | NM_014649.2 | NM_014649 | scaffold attachment | SAFB2 | KIAA0138 |
| | | | | | factor B2 (SAFB2), | | |
| | | | | | mRNA. | | |
| 278 | 243 | 7210035 | NR_003041.1 | NR_003041 | small nucleolar RNA, | SNORD13 | U13 |
| | | | | | C/D box 13 | | |
| | | | | | (SNORD13) on | | |
| | | | | | chromosome 8. | | |
| 279 | 244 | 7210154 | NM_001165.3 | NM_001165 | baculoviral IAP repeat- | BIRC3 | RNF49; MALT2; |
| | | | | | containing 3 (BIRC3), | | MIHC; HAIP1; API2; |
| | | | | | transcript variant 1, | | HIAP1; AIP1; CIAP2 |
| | | | | | mRNA. | | |
| 280 | 245 | 7210326 | NM_004159.4 | NM_004159 | proteasome (prosome, | PSMB8 | D6S216; LMP7; |
| | | | | | macropain) subunit, | | RING10; MGC1491; |
| | | | | | beta type, 8 (large | | D6S216E |
| | | | | | multifunctional | | |
| | | | | | peptidase 7) (PSMB8), | | |
| | | | | | transcript variant 1, | | |
| | | | | | mRNA. | | |
| 281 | 246 | 7210450 | NM_006769.2 | NM_006769 | LIM domain only 4 | LMO4 | — |
| | | | | | (LMO4), mRNA. | | |
| 282 | 247 | 7320041 | NM_015892.2 | NM_015892 | B cell RAG associated | GALNAC4S- | DKFZp781H1369; |
| | | | | | protein (GALNAC4S- | 6ST | KIAA0598; BRAG; |
| | | | | | 6ST), mRNA. | | RP11-47G11.1; |
| | | | | | | | MGC34346 |
| 283 | 248 | 7320551 | NM_002350.1 | NM_002350 | v-yes-1 Yamaguchi | LYN | FLJ26625; JTK8 |
| | | | | | sarcoma viral related | | |
| | | | | | oncogene homolog | | |
| | | | | | (LYN), mRNA. | | |
| 284 | 249 | 7380255 | NM_022481.5 | NM_022481 | centaurin, delta 3 | CENTD3 | FLJ21065; ARAP3; |
| | | | | | (CENTD3), mRNA. | | DRAG1 |
| 285 | 250 | 7400653 | NM_004567.2 | NM_004567 | 6-phosphofructo-2- | PFKFB4 | — |
| | | | | | kinase/fructose-2,6- | | |
| | | | | | biphosphatase 4 | | |
| | | | | | (PFKFB4), mRNA. | | |
| 286 | 251 | 7400673 | NM_001039457.1 | NM_001039457 | ATPase, H+ | ATP6V0B | HATPL; ATP6F; |
| | | | | | transporting, lysosomal | | VMA16 |
| | | | | | 21 kDa, V0 subunit b | | |
| | | | | | (ATP6V0B), transcript | | |
| | | | | | variant 2, mRNA. | | |
| 287 | 252 | 7550364 | NM_001077628.1 | NM_001077628 | anterior pharynx | APH1A | APH-1A; CGI-78; |
| | | | | | defective 1 homolog A | | 6530402N02Rik |
| | | | | | (C. elegans) (APH1A), | | |
| | | | | | transcript variant 1, | | |
| | | | | | mRNA. | | |
| 288 | 253 | 7610187 | NM_182810.1 | NM_182810 | activating transcription | ATF4 | TXREB; TAXREB67; |
| | | | | | factor 4 (tax-responsive | | CREB2; CREB-2 |
| | | | | | enhancer element B67) | | |
| | | | | | (ATF4), transcript | | |
| | | | | | variant 2, mRNA. | | |
| 289 | 255 | 7650209 | NM_001003943.1 | NM_001003943 | Bcl2 modifying factor | BMF | FLJ00065 |
| | | | | | (BMF), transcript | | |
| | | | | | variant 4, mRNA. | | |
| 290 | 256 | 510132 | XM_941861.1 | XM_941861 | PREDICTED: similar to | LOC650029 | — |
| | | | | | RNA-binding protein 4 | | |
| | | | | | (RNA-binding motif | | |
| | | | | | protein 4) (Lark | | |
| | | | | | homolog) (Hlark) (RNA- | | |
| | | | | | binding motif protein | | |
| | | | | | 4a), transcript variant 1 | | |
| | | | | | (LOC650029), mRNA. | | |
| 291 | 257 | 610280 | NM_025029.2 | NM_025029 | family with sequence | FAM128B | FLJ14346; |
| | | | | | similarity 128, member | | MGC87017 |
| | | | | | B (FAM128B), mRNA. | | |
| 292 | 258 | 650129 | BX093310 | BX093310 | BX093310 | — | — |
| | | | | | NCI_CGAP_GC4 | | |
| | | | | | cDNA clone | | |
| | | | | | IMAGp998F143166; | | |
| | | | | | IMAGE: 1257997, | | |
| | | | | | mRNA sequence | | |
| 293 | 259 | 830484 | XM_938599.2 | XM_938599 | PREDICTED: similar to | LOC441377 | — |
| | | | | | 40S ribosomal protein | | |
| | | | | | S26 (LOC441377), | | |
| | | | | | mRNA. | | |
| 294 | 260 | 830639 | XM_929667.1 | XM_929667 | PREDICTED: similar to | LOC653778 | — |
| | | | | | solute carrier family 25, | | |
| | | | | | member 37 | | |
| | | | | | (LOC653778), mRNA. | | |
| 295 | 261 | 870181 | NM_001080544.1 | NM_001080544 | similar to ribosomal | LOC653314 | — |
| | | | | | protein L19 | | |
| | | | | | (LOC653314), mRNA. | | |
| 296 | 262 | 1010039 | AI218425 | AI218425 | qh24c08.x1 | — | — |
| | | | | | Soares_NFL_T_GBC_S1 | | |
| | | | | | cDNA clone | | |
| | | | | | IMAGE: 1845614 3, | | |
| | | | | | mRNA sequence | | |
| 297 | 263 | 1260066 | AK024852 | AK024852 | cDNA: FLJ21199 fis, | — | — |
| | | | | | clone C0L00235 | | |
| 298 | 264 | 1500538 | XM_928168.1 | XM_928168 | PREDICTED: similar to | LOC645138 | — |
| | | | | | ribosomal protein S11 | | |
| | | | | | (LOC645138), mRNA. | | |
| 299 | 265 | 1940274 | NM_032036.2 | NM_032036 | family with sequence | FAM14A | TLH29; MGC44913 |
| | | | | | similarity 14, member A | | |
| | | | | | (FAM14A), mRNA. | | |
| 300 | 266 | 1980112 | NM_197956.1 | NM_197956 | chromosome 9 open | C9orf90 | KIAA1896; |
| | | | | | reading frame 90 | | DKFZp762G199; |
| | | | | | (C9orf90), mRNA. | | RP11-379C10.2; |
| | | | | | | | bA379C10.2 |
| 301 | 267 | 2000564 | NM_001042475.1 | NM_001042475 | chromosome 6 open | C6orf204 | MGC131785; RP11- |
| | | | | | reading frame 204 | | 57K17.2; bA57K17.2; |
| | | | | | (C6orf204), transcript | | NY-BR-15 |
| | | | | | variant 1, mRNA. | | |
| 302 | 268 | 2260025 | XR_015514.1 | XR_015514 | PREDICTED: similar to | LOC730746 | — |
| | | | | | Heterogeneous nuclear | | |
| | | | | | ribonucleoprotein A1 | | |
| | | | | | (Helix-destabilizing | | |
| | | | | | protein) (Single-strand | | |
| | | | | | RNA-binding protein) | | |
| | | | | | (hnRNP core protein | | |
| | | | | | A1 (HDP) | | |
| | | | | | (LOC730746), mRNA. | | |
| 303 | 269 | 2340446 | XM_942351.2 | XM_942351 | PREDICTED: similar to | LOC652726 | — |
| | | | | | ankyrin repeat domain | | |
| | | | | | 36 (LOC652726), | | |
| | | | | | mRNA. | | |
| 304 | 270 | 2470240 | NM_004848.2 | NM_004848 | chromosome 1 open | C1orf38 | ICB-1 |
| | | | | | reading frame 38 | | |
| | | | | | (C1orf38), transcript | | |
| | | | | | variant 1, mRNA. | | |
| 305 | 273 | 2940066 | XM_928429.1 | XM_928429 | PREDICTED: similar to | LOC388275 | — |
| | | | | | Heterogeneous nuclear | | |
| | | | | | ribonucleoprotein A1 | | |
| | | | | | (Helix-destabilizing | | |
| | | | | | protein) (Single-strand | | |
| | | | | | binding protein) | | |
| | | | | | (hnRNP core protein | | |
| | | | | | A1) (HDP-1) | | |
| | | | | | (Topoisomerase- | | |
| | | | | | inhibitor suppressed) | | |
| | | | | | (LOC388275), mRNA. | | |
| 306 | 274 | 2940452 | AK056642 | AK056642 | cDNA FLJ32080 fis, | — | — |
| | | | | | clone OCBBF2000015 | | |
| 307 | 275 | 3190348 | XM_944816.1 | XM_944816 | PREDICTED: similar to | LOC440927 | — |
| | | | | | 60S acidic ribosomal | | |
| | | | | | protein P1, transcript | | |
| | | | | | variant 4 (LOC440927), | | |
| | | | | | mRNA. | | |
| 308 | 276 | 3400709 | AK094914 | AK094914 | cDNA FLJ37595 fis, | — | — |
| | | | | | clone BRCOC2007864 | | |
| 309 | 277 | 3460014 | NM_016613.5 | NM_016613 | chromosome 4 open | C4orf18 | AD021; |
| | | | | | reading frame 18 | | DKFZp434L142; |
| | | | | | (C4orf18), transcript | | AD036; FLJ38155 |
| | | | | | variant 2, mRNA. | | |
| 310 | 278 | 3780148 | NM_024067.2 | NM_024067 | chromosome 7 open | C7orf26 | MGC2718 |
| | | | | | reading frame 26 | | |
| | | | | | (C7orf26), mRNA. | | |
| 311 | 279 | 3850411 | XM_933119.1 | XM_933119 | PREDICTED: similar to | LOC653316 | — |
| | | | | | NY-REN-7 antigen, | | |
| | | | | | transcript variant 4 | | |
| | | | | | (LOC653316), mRNA. | | |
| 312 | 280 | 3870470 | XR_015809.1 | XR_015809 | PREDICTED: similar to | LOC728973 | — |
| | | | | | 40S ribosomal protein | | |
| | | | | | S7 (S8) (LOC728973), | | |
| | | | | | mRNA. | | |
| 313 | 281 | 4060382 | XM_931996.1 | XM_931996 | PREDICTED: similar to | LOC643035 | — |
| | | | | | CG33096-PB, isoform | | |
| | | | | | B, transcript variant 2 | | |
| | | | | | (LOC643035), mRNA. | | |
| 314 | 282 | 4480600 | NM_080757.1 | NM_080757 | chromosome 20 open | C20orf127 | dJ614O4.6; |
| | | | | | reading frame 127 | | MGC118948 |
| | | | | | (C20orf127), mRNA. | | |
| 315 | 283 | 4610681 | XM_939687.2 | XM_939687 | PREDICTED: similar to | LOC653658 | — |
| | | | | | ribosomal protein S23 | | |
| | | | | | (LOC653658), mRNA. | | |
| 316 | 284 | 4860341 | NM_145060.3 | NM_145060 | chromosome 18 open | C18orf24 | MGC10200; Ska1 |
| | | | | | reading frame 24 | | |
| | | | | | (C18orf24), transcript | | |
| | | | | | variant 2, mRNA. | | |
| 317 | 285 | 5310681 | XM_933085.1 | XM_933085 | PREDICTED: similar to | LOC653344 | — |
| | | | | | cis-Golgi matrix protein | | |
| | | | | | GM130, transcript | | |
| | | | | | variant 2 (LOC653344), | | |
| | | | | | mRNA. | | |
| 318 | 286 | 5340278 | XM_932991.1 | XM_932991 | PREDICTED: | LOC643977 | — |
| | | | | | hypothetical protein | | |
| | | | | | LOC643977, transcript | | |
| | | | | | variant 1 (LOC643977), | | |
| | | | | | mRNA. | | |
| 319 | 287 | 5390685 | XM_928197.1 | XM_928197 | PREDICTED: similar to | LOC643433 | — |
| | | | | | 60S ribosomal protein | | |
| | | | | | L29 (Cell surface | | |
| | | | | | heparin binding protein | | |
| | | | | | HIP), transcript variant | | |
| | | | | | 1 (LOC643433), | | |
| | | | | | mRNA. | | |
| 320 | 288 | 5420438 | NM_138471.1 | NM_138471 | hypothetical protein | LOC144097 | DKFZp762N0114 |
| | | | | | BC007540 | | |
| | | | | | (LOC144097), mRNA. | | |
| 321 | 289 | 5420750 | XM_941125.1 | XM_941125 | PREDICTED: similar to | LOC649447 | — |
| | | | | | 60S ribosomal protein | | |
| | | | | | L29 (Cell surface | | |
| | | | | | heparin binding protein | | |
| | | | | | HIP) (LOC649447), | | |
| | | | | | mRNA. | | |
| 322 | 290 | 5490603 | NM_001080831.1 | NM_001080831 | hCG1783417 | LOC401019 | — |
| | | | | | (LOC401019), mRNA. | | |
| 323 | 291 | 5820202 | XR_018325.1 | XR_018325 | PREDICTED: similar to | LOC644131 | — |
| | | | | | chaperonin containing | | |
| | | | | | TCP1, subunit 8 (theta) | | |
| | | | | | (LOC644131), mRNA. | | |
| 324 | 292 | 5890615 | NM_001045478.1 | NM_001045478 | chromosome 1 open | C1orf200 | — |
| | | | | | reading frame 200 | | |
| | | | | | (C1orf200), mRNA. | | |
| 325 | 293 | 5960086 | BP873537 | BP873537 | BP873537 Sugano | — | — |
| | | | | | cDNA library, | | |
| | | | | | embryonal kidney | | |
| | | | | | cDNA clone | | |
| | | | | | HKR13896, mRNA | | |
| | | | | | sequence | | |
| 326 | 294 | 6020066 | XM_940333.2 | XM_940333 | PREDICTED: similar to | LOC651202 | — |
| | | | | | large subunit ribosomal | | |
| | | | | | protein L36a | | |
| | | | | | (LOC651202), mRNA. | | |
| 327 | 295 | 6110195 | NM_153367.2 | NM_153367 | chromosome 10 open | C10orf56 | FLJ90798 |
| | | | | | reading frame 56 | | |
| | | | | | (C10orf56), mRNA. | | |
| 328 | 296 | 6200706 | AA082988 | AA082988 | zn08b06.s1 | — | — |
| | | | | | StratagenehNT neuron | | |
| | | | | | (#937233) cDNA clone | | |
| | | | | | IMAGE: 546803 3, | | |
| | | | | | mRNA sequence | | |
| 329 | 297 | 6270307 | XM_930344.2 | XM_930344 | PREDICTED: similar to | LOC644934 | — |
| | | | | | 40S ribosomal protein | | |
| | | | | | S26, transcript variant 1 | | |
| | | | | | (LOC644934), mRNA. | | |
| 330 | 298 | 6270605 | NR_003040.1 | NR_003040 | ribosomal protein L23a | LOC649946 | — |
| | | | | | pseudogene | | |
| | | | | | (LOC649946) on | | |
| | | | | | chromosome 11. | | |
| 331 | 299 | 6280446 | XM_926370.1 | XM_926370 | PREDICTED: similar to | LOC642989 | — |
| | | | | | 40S ribosomal protein | | |
| | | | | | S25 (LOC642989), | | |
| | | | | | mRNA. | | |
| 332 | 300 | 6280706 | XM_933956.1 | XM_933956 | PREDICTED: similar to | LOC644162 | — |
| | | | | | septin 7, transcript | | |
| | | | | | variant 4 (LOC644162), | | |
| | | | | | mRNA. | | |
| 333 | 301 | 6370288 | XM_938283.2 | XM_938283 | PREDICTED: | C17orf68 | — |
| | | | | | chromosome 17 open | | |
| | | | | | reading frame 68 | | |
| | | | | | (C17orf68), mRNA. | | |
| 334 | 302 | 6480092 | NM_024519.2 | NM_024519 | family with sequence | FAM65A | FLJ13725; KIAA1930 |
| | | | | | similarity 65, member A | | |
| | | | | | (FAM65A), mRNA. | | |
| 335 | 303 | 6510753 | XM_936874.1 | XM_936874 | PREDICTED: similar to | LOC642210 | — |
| | | | | | 60S ribosomal protein | | |
| | | | | | L32 (LOC642210), | | |
| | | | | | mRNA. | | |
| 336 | 304 | 6660753 | NM_017822.3 | NM_017822 | chromosome 12 open | C12orf41 | FLJ20436; FLJ12670 |
| | | | | | reading frame 41 | | |
| | | | | | (C12orf41), mRNA. | | |
| 337 | 305 | 6760202 | NM_001014812.1 | NM_001014812 | family with sequence | FAM96A | FLJ22875 |
| | | | | | similarity 96, member A | | |
| | | | | | (FAM96A), transcript | | |
| | | | | | variant 2, mRNA. | | |
| 338 | 306 | 6840477 | CD640673 | CD640673 | AGENCOURT_14535501 | — | — |
| | | | | | NIH_MGC_191 | | |
| | | | | | cDNA clone | | |
| | | | | | IMAGE: 30415823 5, | | |
| | | | | | mRNA sequence | | |
| 339 | 307 | 6860162 | XM_498969.2 | XM_498969 | PREDICTED: | LOC441019 | — |
| | | | | | hypothetical | | |
| | | | | | LOC441019 | | |
| | | | | | (LOC441019), mRNA. | | |
| 340 | 308 | 7160079 | NM_016623.3 | NM_016623 | family with sequence | FAM49B | L1; |
| | | | | | similarity 49, member B | | DKFZp686B04128; |
| | | | | | (FAM49B), mRNA. | | BM-009 |
| 341 | 309 | 7320707 | XM_939368.1 | XM_939368 | PREDICTED: similar to | LOC654103 | — |
| | | | | | solute carrier family 25, | | |
| | | | | | member 37 | | |
| | | | | | (LOC654103), mRNA. | | |
| 342 | 310 | 7400689 | NM_017896.2 | NM_017896 | chromosome 20 open | C20orf11 | TWA1 |
| | | | | | reading frame 11 | | |
| | | | | | (C20orf11), mRNA. | | |
| 343 | 311 | 7510543 | NM_017924.2 | NM_017924 | chromosome 14 open | C14orf119 | MGC74723; |
| | | | | | reading frame 119 | | FLJ20671 |
| | | | | | (C14orf119), mRNA. | | |
| 344 | 312 | 7610608 | NM_001093763.1 | NM_001093763 | hCG31916 | LOC653702 | — |
| | | | | | (LOC653702), mRNA. | | |
| 345 | 313 | 10201 | NM_017920.3 | NM_017920 | up-regulated gene 4 | URG4 | DKFZp666G166; |
| | | | | | (URG4), nuclear gene | | DKFZp686O0457 |
| | | | | | encoding mitochondrial | | |
| | | | | | protein, transcript | | |
| | | | | | variant 1, mRNA. | | |
| 346 | 314 | 10224 | XM_941466.2 | XM_941466 | PREDICTED: meteorin, | METRNL | — |
| | | | | | glial cell differentiation | | |
| | | | | | regulator-like | | |
| | | | | | (METRNL), mRNA. | | |
| 347 | 315 | 10240 | NM_002558.2 | NM_002558 | purinergic receptor | P2RX1 | P2X1 |
| | | | | | P2X, ligand-gated ion | | |
| | | | | | channel, 1 (P2RX1), | | |
| | | | | | mRNA. | | |
| 348 | 316 | 10286 | NM_025008.3 | NM_025008 | ADAMTS-like 4 | ADAMTSL4 | TSRC1 |
| | | | | | (ADAMTSL4), | | |
| | | | | | transcript variant 2, | | |
| | | | | | mRNA. | | |
| 349 | 317 | 10356 | NR_000016.1 | NR_000016 | small nucleolar RNA, | SNORD36C | RNU36C; U36c |
| | | | | | C/D box 36C | | |
| | | | | | (SNORD36C) on | | |
| | | | | | chromosome 9. | | |
| 350 | 318 | 10594 | NM_006117.2 | NM_006117 | peroxisomal D3,D2- | PECI | DRS1; KIAA0536; |
| | | | | | enoyl-CoA isomerase | | HCA88; |
| | | | | | (PECI), transcript | | dJ1013A10.3; ACBD2 |
| | | | | | variant 1, mRNA. | | |
| 351 | 319 | 10673 | NM_012401.2 | NM_012401 | plexin B2 (PLXNB2), | PLXNB2 | PLEXB2; Nbla00445; |
| | | | | | mRNA. | | MM1; dJ402G11.3; |
| | | | | | | | KIAA0315 |
| 352 | 320 | 20129 | NM_001803.2 | NM_001803 | CD52 molecule | CD52 | CDW52 |
| | | | | | (CD52), mRNA. | | |
| 353 | 321 | 20170 | NM_015481.1 | NM_015481 | zinc finger protein 385A | ZNF385A | DKFZP586G1122; |
| | | | | | (ZNF385A), mRNA. | | RZF; HZF; ZFP385; |
| | | | | | | | ZNF385 |
| 354 | 322 | 20403 | NM_015602.2 | NM_015602 | torsin A interacting | TOR1AIP1 | MGC3413; LAP1B; |
| | | | | | protein 1 (TOR1AIP1), | | DKFZP586G011; |
| | | | | | mRNA. | | RP11-533E19.1; |
| | | | | | | | FLJ13142 |
| 355 | 323 | 20521 | NM_032844.1 | NM_032844 | microtubule associated | MASTL | RP11-85G18.2; |
| | | | | | serine/threonine | | FLJ14813; THC2 |
| | | | | | kinase-like (MASTL), | | |
| | | | | | mRNA. | | |
| 356 | 324 | 50224 | NM_004401.2 | NM_004401 | DNA fragmentation | DFFA | DFF-45; ICAD; DFF1 |
| | | | | | factor, 45 kDa, alpha | | |
| | | | | | polypeptide (DFFA), | | |
| | | | | | transcript variant 1, | | |
| | | | | | mRNA. | | |
| 357 | 325 | 50240 | NM_004542.2 | NM_004542 | NADH dehydrogenase | NDUFA3 | B9 |
| | | | | | (ubiquinone) 1 alpha | | |
| | | | | | subcomplex, 3, 9 kDa | | |
| | | | | | (NDUFA3), mRNA. | | |
| 358 | 326 | 50402 | NM_001975.2 | NM_001975 | enolase 2 (gamma, | ENO2 | NSE |
| | | | | | neuronal) (ENO2), | | |
| | | | | | mRNA. | | |
| 359 | 327 | 50440 | NM_020861.1 | NM_020861 | zinc finger and BTB | ZBTB2 | ZNF437 |
| | | | | | domain containing 2 | | |
| | | | | | (ZBTB2), mRNA. | | |
| 360 | 328 | 50487 | NM_032725.2 | NM_032725 | BUD13 homolog (S. cerevisiae) | BUD13 | MGC13125 |
| | | | | | (BUD13), | | |
| | | | | | mRNA. | | |
| 361 | 329 | 60136 | NM_031311.3 | NM_031311 | carboxypeptidase, | CPVL | HVLP; MGC10029 |
| | | | | | vitellogenic-like (CPVL), | | |
| | | | | | transcript variant 1, | | |
| | | | | | mRNA. | | |
| 362 | 330 | 60148 | NM_001031827.1 | NM_001031827 | bolA homolog 2 (E. coli) | BOLA2 | BOLA2A; My016 |
| | | | | | (BOLA2), mRNA. | | |
| 363 | 331 | 60400 | NM_002887.3 | NM_002887 | arginyl-tRNAsynthetase | RARS | ArgRS; DALRD1; |
| | | | | | (RARS), mRNA. | | MGC8641 |
| 364 | 332 | 60437 | NM_000302.2 | NM_000302 | procollagen-lysine 1,2- | PLOD1 | LLH; PLOD; LH |
| | | | | | oxoglutarate 5- | | |
| | | | | | dioxygenase 1 | | |
| | | | | | (PLOD1), mRNA. | | |
| 365 | 333 | 60647 | NM_201397.1 | NM_201397 | glutathione peroxidase | GPX1 | MGC14399; |
| | | | | | 1 (GPX1), transcript | | GSHPX1; MGC88245 |
| | | | | | variant 2, mRNA. | | |
| 366 | 334 | 70070 | NM_000837.1 | NM_000837 | glutamate receptor, | GRINA | HNRGW; NMDARA1; |
| | | | | | ionotropic, N-methyl D- | | TMBIM3; MGC99687 |
| | | | | | aspartate-associated | | |
| | | | | | protein 1 (glutamate | | |
| | | | | | binding) (GRINA), | | |
| | | | | | transcript variant 1, | | |
| | | | | | mRNA. | | |
| 367 | 335 | 70092 | NR_003655.1 | NR_003655 | polymerase (RNA) II | POLR2J4 | RPB11-phi; |
| | | | | | (DNA directed) | | MGC13098 |
| | | | | | polypeptide J, 13.3 kDa | | |
| | | | | | pseudogene | | |
| | | | | | (POLR2J4) on | | |
| | | | | | chromosome 7. | | |
| 368 | 336 | 70364 | NM_012097.3 | NM_012097 | ADP-ribosylation factor- | ARL5A | ARFLP5; ARL5 |
| | | | | | like 5A (ARL5A), | | |
| | | | | | transcript variant 1, | | |
| | | | | | mRNA. | | |
| 369 | 337 | 70451 | NM_000377.1 | NM_000377 | Wiskott-Aldrich | WAS | WASP; IMD2; THC |
| | | | | | syndrome (eczema- | | |
| | | | | | thrombocytopenia) | | |
| | | | | | (WAS), mRNA. | | |
| 370 | 338 | 70458 | NM_006995.3 | NM_006995 | butyrophilin, subfamily | BTN2A2 | BTF2; BT2.2; |
| | | | | | 2, member A2 | | FLJ41908 |
| | | | | | (BTN2A2), transcript | | |
| | | | | | variant 1, mRNA. | | |
| 371 | 339 | 70541 | NM_005132.2 | NM_005132 | REC8 homolog (yeast) | REC8 | HR21spB; REC8; |
| | | | | | (REC8), transcript | | MGC950; REC8L1; |
| | | | | | variant 1, mRNA. | | Rec8p |
| 372 | 340 | 70630 | NM_182757.2 | NM_182757 | ring finger 144B | RNF144B | KIAA0161; |
| | | | | | (RNF144B), mRNA. | | MGC71786; IBRDC2; |
| | | | | | | | p53RFP; bA528A10.3 |
| 373 | 341 | 110139 | NM_019063.2 | NM_019063 | echinoderm | EML4 | FLJ10942; C2orf2; |
| | | | | | microtubule associated | | ELP120; |
| | | | | | protein like 4 (EML4), | | DKFZp686P18118; |
| | | | | | mRNA. | | ROPP120; FLJ32318 |
| 374 | 342 | 110180 | NM_001080157.1 | NM_001080157 | Rho GTPase activating | ARHGAP9 | 10C; RGL1; |
| | | | | | protein 9 (ARHGAP9), | | FLJ16525; MGC1295 |
| | | | | | transcript variant 2, | | |
| | | | | | mRNA. | | |
| 375 | 343 | 110279 | NM_001037442.1 | NM_001037442 | RUN and FYVE | RUFY3 | SINGAR1; KIAA0871; |
| | | | | | domain containing 3 | | RIPX |
| | | | | | (RUFY3), transcript | | |
| | | | | | variant 1, mRNA. | | |
| 376 | 344 | 110280 | NM_033503.3 | NM_033503 | Bcl2 modifying factor | BMF | FLJ00065 |
| | | | | | (BMF), transcript | | |
| | | | | | variant 2, mRNA. | | |
| 377 | 345 | 110382 | NM_002872.3 | NM_002872 | ras-related C3 | RAC2 | Gx; EN-7; HSPC022 |
| | | | | | botulinum toxin | | |
| | | | | | substrate 2 (rho family, | | |
| | | | | | small GTP binding | | |
| | | | | | protein Rac2) (RAC2), | | |
| | | | | | mRNA. | | |
| 378 | 346 | 110445 | NM_001005744.1 | NM_001005744 | numb homolog | NUMB | S171 |
| | | | | | (Drosophila) (NUMB), | | |
| | | | | | transcript variant 2, | | |
| | | | | | mRNA. | | |
| 379 | 347 | 110661 | NM_001025235.1 | NM_001025235 | tetraspanin 4 | TSPAN4 | NAG-2; NAG2; |
| | | | | | (TSPAN4), transcript | | TETRASPAN; |
| | | | | | variant 3, mRNA. | | TM4SF7; TSPAN-4 |
| 380 | 348 | 110682 | NM_005620.1 | NM_005620 | S100 calcium binding | S100A11 | S100C; MLN70 |
| | | | | | protein A11 (S100A11), | | |
| | | | | | mRNA. | | |
| 381 | 349 | 110685 | NM_003177.3 | NM_003177 | spleen tyrosine kinase | SYK | — |
| | | | | | (SYK), mRNA. | | |
| 382 | 350 | 110739 | NM_032164.2 | NM_032164 | zinc finger protein 394 | ZNF394 | FLJ12298; |
| | | | | | (ZNF394), mRNA. | | ZKSCAN14 |
| 383 | 352 | 130338 | NM_000331.3 | NM_000331 | serum amyloid A1 | SAA1 | MGC111216; SAA; |
| | | | | | (SAA1), transcript | | PIG4; TP53I4 |
| | | | | | variant 1, mRNA. | | |
| 384 | 354 | 130382 | NM_018957.3 | NM_018957 | SH3-domain binding | SH3BP1 | — |
| | | | | | protein 1 (SH3BP1), | | |
| | | | | | mRNA. | | |
| 385 | 355 | 130593 | NM_024787.2 | NM_024787 | ring finger protein 122 | RNF122 | MGC126622; |
| | | | | | (RNF122), mRNA. | | FLJ12526 |
| 386 | 356 | 130603 | NM_014183.2 | NM_014183 | dynein, light chain, | DYNLRB1 | BLP; ROBLD1; BITH; |
| | | | | | roadblock-type 1 | | DNLC2A; DNCL2A |
| | | | | | (DYNLRB1), mRNA. | | |
| 387 | 357 | 130669 | NM_004873.2 | NM_004873 | BCL2-associated | BAG5 | BAG-5 |
| | | | | | athanogene 5 (BAG5), | | |
| | | | | | transcript variant 2, | | |
| | | | | | mRNA. | | |
| 388 | 358 | 150048 | NM_001042734.1 | NM_001042734 | SEC24 related gene | SEC24B | MGC48822; SEC24 |
| | | | | | family, member B (S. cerevisiae) | | |
| | | | | | (SEC24B), | | |
| | | | | | transcript variant 2, | | |
| | | | | | mRNA. | | |
| 389 | 359 | 150072 | NM_002163.2 | NM_002163 | interferon regulatory | IRF8 | H-ICSBP; ICSBP; |
| | | | | | factor 8 (IRF8), mRNA. | | IRF-8; ICSBP1 |
| 390 | 360 | 150095 | NM_001031685.2 | NM_001031685 | tumor protein p53 | TP53BP2 | 53BP2; BBP; |
| | | | | | binding protein, 2 | | p53BP2; ASPP2; |
| | | | | | (TP53BP2), transcript | | PPP1R13A |
| | | | | | variant 1, mRNA. | | |
| 391 | 361 | 160019 | NM_002959.4 | NM_002959 | sortilin 1 (SORT1), | SORT1 | NT3; Gp95 |
| | | | | | mRNA. | | |
| 392 | 362 | 160068 | NM_015621.2 | NM_015621 | coiled-coil domain | CCDC69 | DKFZP434C171; |
| | | | | | containing 69 | | FLJ13705 |
| | | | | | (CCDC69), mRNA. | | |
| 393 | 363 | 160279 | NM_145648.1 | NM_145648 | solute carrier family 15, | SLC15A4 | PHT1; FP12591; |
| | | | | | member 4 (SLC15A4), | | PTR4 |
| | | | | | mRNA. | | |
| 394 | 364 | 160368 | NM_032108.2 | NM_032108 | sema domain, | SEMA6B | SEMAN; semaZ; |
| | | | | | transmembrane | | SEM-SEMA-Y; |
| | | | | | domain (TM), and | | SEMA-VIB; SemaVIb |
| | | | | | cytoplasmic domain, | | |
| | | | | | (semaphorin) 6B | | |
| | | | | | (SEMA6B), mRNA. | | |
| 395 | 365 | 160494 | NM_020980.2 | NM_020980 | aquaporin 9 (AQP9), | AQP9 | HsT17287; SSC1 |
| | | | | | mRNA. | | |
| 396 | 366 | 160561 | NM_018054.4 | NM_018054 | Rho GTPase activating | ARHGAP17 | FLJ37567; MSTP110; |
| | | | | | protein 17 | | DKFZp564A1363; |
| | | | | | (ARHGAP17), | | MSTP066; RICH1; |
| | | | | | transcript variant 2, | | MST066; FLJ43368; |
| | | | | | mRNA. | | MGC87805; |
| | | | | | | | MSTP038; WBP15; |
| | | | | | | | NADRIN; MST110 |
| 397 | 367 | 160731 | NM_016479.3 | NM_016479 | shisa homolog 5 | SHISA5 | — |
| | | | | | (Xenopuslaevis) | | |
| | | | | | (SHISA5), mRNA. | | |
| 398 | 368 | 160746 | NM_000485.2 | NM_000485 | adenine | APRT | MGC125857; AMP; |
| | | | | | phosphoribosyltransferase | | MGC125856; |
| | | | | | (APRT), transcript | | DKFZp686D13177; |
| | | | | | variant 1, mRNA. | | MGC129961 |
| 399 | 369 | 240348 | NM_014634.2 | NM_014634 | protein phosphatase 1F | PPM1F | POPX2; CaMKPase; |
| | | | | | (PP2C domain | | FEM-2; hFEM-2; |
| | | | | | containing) (PPM1F), | | KIAA0015 |
| | | | | | mRNA. | | |
| 400 | 370 | 240435 | NM_018994.1 | NM_018994 | F-box protein 42 | FBXO42 | Fbx42; KIAA1332 |
| | | | | | (FBXO42), mRNA. | | |
| 401 | 371 | 270224 | NM_022151.4 | NM_022151 | modulator of apoptosis | MOAP1 | MAP-1; PNMA4 |
| | | | | | 1 (MOAP1), mRNA. | | |
| 402 | 372 | 270619 | NM_002097.1 | NM_002097 | general transcription | GTF3A | AP2; TFIIIA |
| | | | | | factor IIIA (GTF3A), | | |
| | | | | | mRNA. | | |
| 403 | 373 | 290750 | NM_001017373.1 | NM_001017373 | sterile alpha motif | SAMD3 | MGC35163; |
| | | | | | domain containing 3 | | FLJ34563 |
| | | | | | (SAMD3), transcript | | |
| | | | | | variant 1, mRNA. | | |
| 404 | 375 | 360482 | NM_015383.1 | NM_015383 | neuroblastoma | NBPF14 | RP3-328E19.1; |
| | | | | | breakpoint family, | | DJ328E19.C1.1; |
| | | | | | member 14 (NBPF14), | | NBPF; FLJ35032 |
| | | | | | mRNA. | | |
| 405 | 376 | 360619 | NM_005248.2 | NM_005248 | Gardner-Rasheed | FGR | p58c-fgr, SRC2; c-fgr; |
| | | | | | feline sarcoma viral (v- | | FLJ43153; |
| | | | | | fgr) oncogene homolog | | MGC75096; c-src2; |
| | | | | | (FGR), transcript | | p55c-fgr |
| | | | | | variant 1, mRNA. | | |
| 406 | 377 | 360719 | NM_000610.3 | NM_000610 | CD44 molecule (Indian | CD44 | LHR; MDU2; CDW44; |
| | | | | | blood group) (CD44), | | CSPG8; Pgp1; IN; |
| | | | | | transcript variant 1, | | MUTCH-I; MIC4; |
| | | | | | mRNA. | | MDU3; MC56; |
| | | | | | | | HCELL; ECMR-III; |
| | | | | | | | MGC10468 |
| 407 | 378 | 360753 | NM_014160.3 | NM_014160 | makorin, ring finger | MKRN2 | HSPC070; RNF62 |
| | | | | | protein, 2 (MKRN2), | | |
| | | | | | mRNA. | | |
| 408 | 379 | 380050 | NM_024599.3 | NM_024599 | rhomboid 5 homolog 2 | RHBDF2 | RHBDL6; RHBDL5; |
| | | | | | (Drosophila) | | FLJ22341 |
| | | | | | (RHBDF2), transcript | | |
| | | | | | variant 1, mRNA. | | |
| 409 | 380 | 380056 | NM_006577.5 | NM_006577 | UDP-GlcNAc:betaGal | B3GNT2 | B3GNT-2; B3GN-T1; |
| | | | | | beta-1,3-N- | | B3GNT1; |
| | | | | | acetylglucosaminyltransferase | | BETA3GNT; B3GN- |
| | | | | | 2 (B3GNT2), | | T2; B3GNT |
| | | | | | mRNA. | | |
| 410 | 381 | 380392 | NM_001012424.1 | NM_001012424 | YY1 associated factor 2 | YAF2 | MGC41856 |
| | | | | | (YAF2), transcript | | |
| | | | | | variant 2, mRNA. | | |
| 411 | 382 | 380753 | NM_001012456.1 | NM_001012456 | Sec61 gamma subunit | SEC61G | SSS1 |
| | | | | | (SEC61G), transcript | | |
| | | | | | variant 2, mRNA. | | |
| 412 | 383 | 430044 | NM_001033858.1 | NM_001033858 | DNA cross-link repair | DCLRE1C | SCIDA; DCLREC1C; |
| | | | | | 1C (PSO2 homolog, S. cerevisiae) | | SNM1C; FLJ36438; |
| | | | | | (DCLRE1C), transcript | | RS-SCID; FLJ11360; |
| | | | | | variant c, mRNA. | | A-SCID |
| 413 | 384 | 430546 | NM_003518.3 | NM_003518 | histone cluster 1, H2bg | HIST1H2BG | H2B.1A; H2BFA; |
| | | | | | (HIST1H2BG), mRNA. | | dJ221C16.8; |
| | | | | | | | HIST1H2BI; H2B/a; |
| | | | | | | | HIST1H2BF |
| 414 | 385 | 430672 | NM_153201.1 | NM_153201 | heat shock 70 kDa | HSPA8 | MGC131511; HSC54; |
| | | | | | protein 8 (HSPA8), | | HSPA10; MGC29929; |
| | | | | | transcript variant 2, | | HSC70; HSP71; |
| | | | | | mRNA. | | HSP73; LAP1; |
| | | | | | | | HSC71; NIP71 |
| 415 | 386 | 450204 | NM_001077692.1 | NM_001077692 | asparagine-linked | ALG9 | DKFZp586M2420; |
| | | | | | glycosylation 9 | | FLJ21845; DIBD1 |
| | | | | | homolog (S. cerevisiae, | | |
| | | | | | alpha-1,2- | | |
| | | | | | mannosyltransferase) | | |
| | | | | | (ALG9), transcript | | |
| | | | | | variant 4, mRNA. | | |
| 416 | 387 | 460164 | NR_002204.1 | NR_002204 | ferritin, heavy | FTHL11 | — |
| | | | | | polypeptide-like 11 | | |
| | | | | | (FTHL11) on | | |
| | | | | | chromosome 8. | | |
| 417 | 388 | 460204 | NM_001122.2 | NM_001122 | adipose differentiation- | ADFP | ADRP; MGC10598 |
| | | | | | related protein (ADFP), | | |
| | | | | | mRNA. | | |
| 418 | 389 | 460333 | NM_199436.1 | NM_199436 | spastin (SPAST), | SPAST | FSP2; ADPSP; |
| | | | | | transcript variant 2, | | KIAA1083; SPG4 |
| | | | | | mRNA. | | |
| 419 | 390 | 460438 | NM_152872.1 | NM_152872 | Fas (TNF receptor | FAS | CD95; APT1; FASTM; |
| | | | | | superfamily, member 6) | | FAS1; APO-1; |
| | | | | | (FAS), transcript variant | | ALPS1A; TNFRSF6 |
| | | | | | 3, mRNA. | | |
| 420 | 391 | 460463 | NM_001003802.1 | NM_001003802 | SWI/SNF related, | SMARCD3 | Rsc6p; BAF60C; |
| | | | | | matrix associated, actin | | CRACD3; |
| | | | | | dependent regulator of | | MGC111010 |
| | | | | | chromatin, subfamily d, | | |
| | | | | | member 3 | | |
| | | | | | (SMARCD3), transcript | | |
| | | | | | variant 1, mRNA. | | |
| 421 | 392 | 460468 | NM_014169.2 | NM_014169 | chromatin modifying | CHMP4A | SNF7-1; C14orf123; |
| | | | | | protein 4A (CHMP4A), | | SNF7; MGC142095; |
| | | | | | mRNA. | | Shax2; CHMP4B; |
| | | | | | | | MGC142093; |
| | | | | | | | HSPC134 |
| 422 | 393 | 460543 | NM_014683.2 | NM_014683 | unc-51-like kinase 2 (C. elegans) | ULK2 | KIAA0623; Unc51.2 |
| | | | | | (ULK2), | | |
| | | | | | mRNA. | | |
| 423 | 394 | 510114 | NM_182851.1 | NM_182851 | cyclin B1 interacting | CCNB1IP1 | C14orf18; HEI10 |
| | | | | | protein 1 (CCNB1IP1), | | |
| | | | | | transcript variant 3, | | |
| | | | | | mRNA. | | |
| 424 | 395 | 510128 | NM_173653.1 | NM_173653 | solute carrier family 9 | SLC9A9 | NHE9; Nbla00118; |
| | | | | | (sodium/hydrogen | | FLJ35613 |
| | | | | | exchanger), member 9 | | |
| | | | | | (SLC9A9), mRNA. | | |
| 425 | 396 | 510129 | NM_006594.1 | NM_006594 | adaptor-related protein | AP4B1 | BETA-4 |
| | | | | | complex 4, beta 1 | | |
| | | | | | subunit (AP4B1), | | |
| | | | | | mRNA. | | |
| 426 | 397 | 510288 | NM_022736.1 | NM_022736 | major facilitator | MFSD1 | UG0581B09; |
| | | | | | superfamily domain | | FLJ14153 |
| | | | | | containing 1 (MFSD1), | | |
| | | | | | mRNA. | | |
| 427 | 398 | 510291 | NM_001359.1 | NM_001359 | 2,4-dienoyl CoA | DECR1 | NADPH; DECR |
| | | | | | reductase 1, | | |
| | | | | | mitochondrial (DECR1), | | |
| | | | | | nuclear gene encoding | | |
| | | | | | mitochondrial protein, | | |
| | | | | | mRNA. | | |
| 428 | 399 | 510300 | NM_001663.2 | NM_001663 | ADP-ribosylation factor | ARF6 | — |
| | | | | | 6 (ARF6), mRNA. | | |
| 429 | 400 | 510468 | NM_006163.1 | NM_006163 | nuclear factor | NFE2 | p45; NF-E2 |
| | | | | | (erythroid-derived 2), | | |
| | | | | | 45 kDa (NFE2), mRNA. | | |
| 430 | 401 | 520324 | NM_005669.4 | NM_005669 | receptor accessory | REEP5 | MGC70440; DP1; |
| | | | | | protein 5 (REEP5), | | D5S346; TB2; |
| | | | | | mRNA. | | C5orf18 |
| 431 | 402 | 520360 | NM_152851.1 | NM_152851 | membrane-spanning 4- | MS4A6A | CDA01; MSTP090; |
| | | | | | domains, subfamily A, | | MGC22650; 4SPAN3; |
| | | | | | member 6A (MS4A6A), | | 4SPAN3.2; MS4A6; |
| | | | | | transcript variant 3, | | CD20L3; MST090; |
| | | | | | mRNA. | | MGC131944 |
| 432 | 403 | 520408 | NM_001549.2 | NM_001549 | interferon-induced | IFIT3 | IRG2; RIG-G; IFI60; |
| | | | | | protein with | | ISG60; IFIT4; GARG- |
| | | | | | tetratricopeptide | | 49; CIG-49 |
| | | | | | repeats 3 (IFIT3), | | |
| | | | | | mRNA. | | |
| 433 | 404 | 540400 | NM_001048216.1 | NM_001048216 | cementum protein 1 | CEMP1 | CP-23 |
| | | | | | (CEMP1), mRNA. | | |
| 434 | 405 | 540403 | NM_182492.1 | NM_182492 | low density lipoprotein | LRP5L | DKFZp434O0213 |
| | | | | | receptor-related protein | | |
| | | | | | 5-like (LRP5L), mRNA. | | |
| 435 | 407 | 540600 | NM_153827.3 | NM_153827 | misshapen-like kinase | MINK1 | YSK2; hMINK; ZC3; |
| | | | | | 1 (zebrafish) (MINK1), | | MGC21111; MINK; |
| | | | | | transcript variant 3, | | MAP4K6; B55; |
| | | | | | mRNA. | | hMINKbeta |
| 436 | 408 | 540717 | NM_004793.2 | NM_004793 | Ion peptidase 1, | LONP1 | LonHS; hLON; LONP; |
| | | | | | mitochondrial (LONP1), | | MGC1498; PRSS15; |
| | | | | | nuclear gene encoding | | PIM1; LON |
| | | | | | mitochondrial protein, | | |
| | | | | | mRNA. | | |
| 437 | 409 | 540725 | NM_005520.1 | NM_005520 | heterogeneous nuclear | HNRPH1 | DKFZp686A15170; |
| | | | | | ribonucleoprotein H1 | | HNRPH; hnRNPH |
| | | | | | (H) (HNRPH1), mRNA. | | |
| 438 | 410 | 580114 | NM_030768.2 | NM_030768 | integrin-linked kinase- | ILKAP | PP2C-DELTA; |
| | | | | | associated | | FLJ10181; MGC4846; |
| | | | | | serine/threonine | | DKFZP434J2031 |
| | | | | | phosphatase 2C | | |
| | | | | | (ILKAP), mRNA. | | |
| 439 | 411 | 580278 | NM_175887.2 | NM_175887 | proline rich 15 | PRR15 | — |
| | | | | | (PRR15), mRNA. | | |
| 440 | 412 | 580411 | NM_017773.2 | NM_017773 | lymphocyte | LAX1 | LAX; FLJ20340 |
| | | | | | transmembrane | | |
| | | | | | adaptor 1 (LAX1), | | |
| | | | | | mRNA. | | |
| 441 | 413 | 580601 | NM_153747.1 | NM_153747 | phosphatidylinositol | PIGC | GPI2; MGC2049 |
| | | | | | glycan anchor | | |
| | | | | | biosynthesis, class C | | |
| | | | | | (PIGC), transcript | | |
| | | | | | variant 1, mRNA. | | |
| 442 | 414 | 580670 | NM_031443.3 | NM_031443 | cerebral cavernous | CCM2 | MGC4067; |
| | | | | | malformation 2 | | MGC4607; C7orf22; |
| | | | | | (CCM2), transcript | | PP10187; MGC74868 |
| | | | | | variant 2, mRNA. | | |
| 443 | 415 | 610040 | NM_002912.1 | NM_002912 | REV3-like, catalytic | REV3L | POLZ; REV3 |
| | | | | | subunit of DNA | | |
| | | | | | polymerase zeta | | |
| | | | | | (yeast) (REV3L), | | |
| | | | | | mRNA. | | |
| 444 | 416 | 610110 | NM_001989.3 | NM_001989 | even-skipped | EVX1 | — |
| | | | | | homeobox 1 (EVX1), | | |
| | | | | | mRNA. | | |
| 445 | 417 | 610221 | NM_017626.3 | NM_017626 | DnaJ (Hsp40) homolog, | DNAJB12 | DJ10; |
| | | | | | subfamily B, member | | DKFZp586B2023 |
| | | | | | 12 (DNAJB12), | | |
| | | | | | transcript variant 2, | | |
| | | | | | mRNA. | | |
| 446 | 418 | 610367 | NM_016217.2 | NM_016217 | headcase homolog | HECA | HDC; HDCL; |
| | | | | | (Drosophila) (HECA), | | dJ225E12.1; HHDC |
| | | | | | mRNA. | | |
| 447 | 419 | 610689 | NM_003977.1 | NM_003977 | aryl hydrocarbon | AIP | ARA9; FKBP16; |
| | | | | | receptor interacting | | XAP2; SMTPHN; |
| | | | | | protein (AIP), mRNA. | | FKBP37 |
| 448 | 420 | 620019 | XM_376787.4 | XM_376787 | PREDICTED: | RPS26P10 | — |
| | | | | | ribosomal protein S26 | | |
| | | | | | pseudogene 10 | | |
| | | | | | (RPS26P10), mRNA. | | |
| 449 | 421 | 620064 | NM_001037553.1 | NM_001037553 | 1-acylglycerol-3- | AGPAT3 | MGC4604; LPAAT- |
| | | | | | phosphate O- | | GAMMA1 |
| | | | | | acyltransferase 3 | | |
| | | | | | (AGPAT3), transcript | | |
| | | | | | variant 2, mRNA. | | |
| 450 | 422 | 620072 | NM_016544.1 | NM_016544 | rab and DnaJ domain | RBJ | RabJS; |
| | | | | | containing (RBJ), | | DKFZp434N211 |
| | | | | | mRNA. | | |
| 451 | 423 | 620376 | NM_021959.2 | NM_021959 | protein phosphatase 1, | PPP1R11 | TCTEX5; TCTE5; |
| | | | | | regulatory (inhibitor) | | HCGV; MGC125741; |
| | | | | | subunit 11 (PPP1R11), | | HCG-V; MGC125742; |
| | | | | | mRNA. | | MGC125743 |
| 452 | 424 | 620450 | NM_024570.1 | NM_024570 | ribonuclease H2, | RNASEH2B | DLEU8; AGS2; |
| | | | | | subunit B | | FLJ11712 |
| | | | | | (RNASEH2B), mRNA. | | |
| 453 | 425 | 620682 | NM_016612.2 | NM_016612 | solute carrier family 25, | SLC25A37 | PRO2217; PRO1278; |
| | | | | | member 37 | | MFRN; PRO1584; |
| | | | | | (SLC25A37), nuclear | | HT015; MSCP; MSC |
| | | | | | gene encoding | | |
| | | | | | mitochondrial protein, | | |
| | | | | | mRNA. | | |
| 454 | 426 | 620731 | NM_006415.2 | NM_006415 | serine | SPTLC1 | HSAN; HSAN1; |
| | | | | | palmitoyltransferase, | | SPT1; HSN1; |
| | | | | | long chain base subunit | | MGC14645; LBC1; |
| | | | | | 1 (SPTLC1), transcript | | SPTI; LCB1 |
| | | | | | variant 1, mRNA. | | |
| 455 | 427 | 620754 | NM_001009.3 | NM_001009 | ribosomal protein S5 | RPS5 | — |
| | | | | | (RPS5), mRNA. | | |
| 456 | 428 | 620767 | NM_004927.2 | NM_004927 | mitochondrial ribosomal | MRPL49 | NOF; NOF1; C11orf4; |
| | | | | | protein L49 (MRPL49), | | MGC10656; L49mt |
| | | | | | nuclear gene encoding | | |
| | | | | | mitochondrial protein, | | |
| | | | | | mRNA. | | |
| 457 | 429 | 630142 | NM_004468.3 | NM_004468 | four and a half LIM | FHL3 | MGC23614; SLIM2; |
| | | | | | domains 3 (FHL3), | | MGC8696; |
| | | | | | mRNA. | | MGC19547 |
| 458 | 430 | 630609 | NM_022166.3 | NM_022166 | xylosyltransferase I | XYLT1 | XT1; XT-I |
| | | | | | (XYLT1), mRNA. | | |
| 459 | 431 | 630709 | NM_032377.3 | NM_032377 | elongation factor 1 | ELOF1 | ELF1 |
| | | | | | homolog (S. cerevisiae) | | |
| | | | | | (ELOF1), mRNA. | | |
| 460 | 432 | 650020 | NM_203446.1 | NM_203446 | synaptojanin 1 | SYNJ1 | INPP5G |
| | | | | | (SYNJ1), transcript | | |
| | | | | | variant 2, mRNA. | | |
| 461 | 433 | 650040 | NM_178272.1 | NM_178272 | paired immunoglobin- | PILRA | FDF03 |
| | | | | | like type 2 receptor | | |
| | | | | | alpha (PILRA), | | |
| | | | | | transcript variant 2, | | |
| | | | | | mRNA. | | |
| 462 | 434 | 650678 | NM_003186.3 | NM_003186 | transgelin (TAGLN), | TAGLN | TAGLN1; WS3-10; |
| | | | | | transcript variant 2, | | SM22; |
| | | | | | mRNA. | | DKFZp686P11128; |
| | | | | | | | SMCC |
| 463 | 435 | 650692 | NM_207332.1 | NM_207332 | glutamate-rich 1 | ERICH1 | HSPC319 |
| | | | | | (ERICH1), mRNA. | | |
| 464 | 436 | 670025 | NM_014325.2 | NM_014325 | coronin, actin binding | CORO1C | HCRNN4; coronin-3 |
| | | | | | protein, 1C (CORO1C), | | |
| | | | | | transcript variant 1, | | |
| | | | | | mRNA. | | |
| 465 | 437 | 670088 | NM_015204.1 | NM_015204 | thrombospondin, type I, | THSD7A | KIAA0960 |
| | | | | | domain containing 7A | | |
| | | | | | (THSD7A), mRNA. | | |
| 466 | 438 | 670161 | NM_001042445.1 | NM_001042445 | calpastatin (CAST), | CAST | MGC9402; BS-17 |
| | | | | | transcript variant 11, | | |
| | | | | | mRNA. | | |
| 467 | 439 | 670754 | NM_001017970.2 | NM_001017970 | transmembrane protein | TMEM30B | CDC50B; |
| | | | | | 30B (TMEM30B), | | MGC126775 |
| | | | | | mRNA. | | |
| 468 | 440 | 730047 | NM_001002236.1 | NM_001002236 | serpin peptidase | SERPINA1 | PI1; MGC23330; |
| | | | | | inhibitor, clade A | | PRO2275; A1AT; |
| | | | | | (alpha-1 antiproteinase, | | AAT; MGC9222; PI; |
| | | | | | antitrypsin), member 1 | | A1A |
| | | | | | (SERPINA1), transcript | | |
| | | | | | variant 2, mRNA. | | |
| 469 | 441 | 730092 | NM_213650.1 | NM_213650 | sideroflexin 4 (SFXN4), | SFXN4 | BCRM1 |
| | | | | | transcript variant 3, | | |
| | | | | | mRNA. | | |
| 470 | 442 | 730156 | NM_001099786.1 | NM_001099786 | intercellular adhesion | ICAM2 | CD102 |
| | | | | | molecule 2 (ICAM2), | | |
| | | | | | transcript variant 1, | | |
| | | | | | mRNA. | | |
| 471 | 443 | 730288 | NM_031431.2 | NM_031431 | component of | COG3 | SEC34 |
| | | | | | oligomericgolgi | | |
| | | | | | complex 3 (COG3), | | |
| | | | | | mRNA. | | |
| 472 | 444 | 730458 | NM_002333.1 | NM_002333 | low density lipoprotein | LRP3 | — |
| | | | | | receptor-related protein | | |
| | | | | | 3 (LRP3), mRNA. | | |
| 473 | 445 | 730632 | NM_001013255.1 | NM_001013255 | lymphocyte-specific | LSP1 | WP34; pp52 |
| | | | | | protein 1 (LSP1), | | |
| | | | | | transcript variant 4, | | |
| | | | | | mRNA. | | |
| 474 | 446 | 770128 | NM_032438.1 | NM_032438 | I(3)mbt-like 3 | L3MBTL3 | MBT1; MBT-1; RP11- |
| | | | | | (Drosophila) | | 73O6.1 |
| | | | | | (L3MBTL3), transcript | | |
| | | | | | variant 1, mRNA. | | |
| 475 | 447 | 770440 | NM_003565.1 | NM_003565 | unc-51-like kinase 1 (C. elegans) | ULK1 | FLJ38455; UNC51; |
| | | | | | (ULK1), | | Unc51.1; ATG1 |
| | | | | | mRNA. | | |
| 476 | 448 | 770543 | NM_001930.2 | NM_001930 | deoxyhypusine | DHPS | MIG13 |
| | | | | | synthase (DHPS), | | |
| | | | | | transcript variant 1, | | |
| | | | | | mRNA. | | |
| 477 | 449 | 770630 | NM_001008661.1 | NM_001008661 | cysteine conjugate-beta | CCBL2 | DKFZp547N1117; |
| | | | | | lyase 2 (CCBL2), | | RBM1; RP11- |
| | | | | | transcript variant 1, | | 82K18.3; MGC9398; |
| | | | | | mRNA. | | RP4-531M19.2; |
| | | | | | | | RBMXL1; |
| | | | | | | | DKFZp667D0223; |
| | | | | | | | KAT3 |
| 478 | 450 | 770703 | NM_004840.2 | NM_004840 | Rac/Cdc42 guanine | ARHGEF6 | COOL2; PIXA; |
| | | | | | nucleotide exchange | | MRX46; KIAA0006; |
| | | | | | factor (GEF) 6 | | alpha-PIX; Cool-2; |
| | | | | | (ARHGEF6), mRNA. | | alphaPIX |
| 479 | 451 | 770754 | NM_025250.2 | NM_025250 | tweety homolog 3 | TTYH3 | KIAA1691 |
| | | | | | (Drosophila) (TTYH3), | | |
| | | | | | mRNA. | | |
| 480 | 452 | 780324 | NM_015655.2 | NM_015655 | zinc finger protein 337 | ZNF337 | — |
| | | | | | (ZNF337), mRNA. | | |
| 481 | 453 | 780376 | NM_000390.2 | NM_000390 | choroideremia (Rab | CHM | TCD; REP-1; |
| | | | | | escort protein 1) | | FLJ38564; DXS540; |
| | | | | | (CHM), transcript | | MGC102710; GGTA; |
| | | | | | variant 1, mRNA. | | HSD-32 |
| 482 | 454 | 780471 | NM_001031665.1 | NM_001031665 | zinc finger protein 816A | ZNF816A | MGC125619 |
| | | | | | (ZNF816A), mRNA. | | |
| 483 | 455 | 780519 | NM_005586.2 | NM_005586 | MyoD family inhibitor | MDFI | I-MF |
| | | | | | (MDFI), mRNA. | | |
| 484 | 456 | 830047 | NM_000852.2 | NM_000852 | glutathione S- | GSTP1 | DFN7; GST3; PI; |
| | | | | | transferase pi (GSTP1), | | FAEES3 |
| | | | | | mRNA. | | |
| 485 | 458 | 830403 | NM_002818.2 | NM_002818 | proteasome (prosome, | PSME2 | PA28beta; REGbeta; |
| | | | | | macropain) activator | | PA28B |
| | | | | | subunit 2 (PA28 beta) | | |
| | | | | | (PSME2), mRNA. | | |
| 486 | 460 | 830524 | NM_182646.1 | NM_182646 | cytoplasmic | CPEB2 | MGC119575; |
| | | | | | polyadenylation | | MGC119576; |
| | | | | | element binding protein | | MGC119577 |
| | | | | | 2 (CPEB2), transcript | | |
| | | | | | variant A, mRNA. | | |
| 487 | 462 | 830563 | NM_001001567.1 | NM_001001567 | phosphodiesterase 9A | PDE9A | HSPDE9A2 |
| | | | | | (PDE9A), transcript | | |
| | | | | | variant 2, mRNA. | | |
| 488 | 463 | 830653 | NM_001493.1 | NM_001493 | GDP dissociation | GDI1 | OPHN2; MRX48; |
| | | | | | inhibitor 1 (GDI1), | | RABGD1A; GDIL; |
| | | | | | mRNA. | | XAP-4; RABGDIA; |
| | | | | | | | MRX41; FLJ41411 |
| 489 | 464 | 830735 | NM_006869.1 | NM_006869 | centaurin, alpha 1 | CENTA1 | GCS1L; p42IP4 |
| | | | | | (CENTA1), mRNA. | | |
| 490 | 465 | 840168 | NM_000397.2 | NM_000397 | cytochrome b-245, beta | CYBB | CGD; GP91-PHOX; |
| | | | | | polypeptide (chronic | | GP91PHOX; NOX2; |
| | | | | | granulomatous | | GP91-1 |
| | | | | | disease) (CYBB), | | |
| | | | | | mRNA. | | |
| 491 | 466 | 840253 | NM_000690.2 | NM_000690 | aldehyde | ALDH2 | ALDM; ALDH-E2; |
| | | | | | dehydrogenase 2 | | ALDHI; MGC1806 |
| | | | | | family (mitochondrial) | | |
| | | | | | (ALDH2), nuclear gene | | |
| | | | | | encoding mitochondrial | | |
| | | | | | protein, mRNA. | | |
| 492 | 467 | 840358 | NM_002685.2 | NM_002685 | exosome component | EXOSC10 | PMSCL2; PM-Scl; |
| | | | | | 10 (EXOSC10), | | PM/Scl-100; PMSCL; |
| | | | | | transcript variant 2, | | RRP6; p4; Rrp6p; p2; |
| | | | | | mRNA. | | p3 |
| 493 | 468 | 840402 | NM_015001.2 | NM_015001 | spen homolog, | SPEN | RP1-134O19.1; |
| | | | | | transcriptional regulator | | MINT; KIAA0929; |
| | | | | | (Drosophila) (SPEN), | | SHARP |
| | | | | | mRNA. | | |
| 494 | 469 | 840543 | NR_001588.1 | NR_001588 | Shwachman-Bodian- | SBDSP | — |
| | | | | | Diamond syndrome | | |
| | | | | | pseudogene (SBDSP) | | |
| | | | | | on chromosome 7. | | |
| 495 | 470 | 840544 | NM_002896.2 | NM_002896 | RNA binding motif | RBM4 | ZCRB3A; ZCCHC21; |
| | | | | | protein 4 (RBM4), | | RBM4A; LARK; |
| | | | | | mRNA. | | MGC75138; |
| | | | | | | | DKFZp547K0918 |
| 496 | 471 | 840554 | NM_012234.4 | NM_012234 | RING1 and YY1 | RYBP | DEDAF; YEAF1; |
| | | | | | binding protein (RYBP), | | AAP1 |
| | | | | | mRNA. | | |
| 497 | 472 | 840647 | NM_033643.2 | NM_033643 | ribosomal protein L36 | RPL36 | DKFZP566B023 |
| | | | | | (RPL36), transcript | | |
| | | | | | variant 1, mRNA. | | |
| 498 | 473 | 870324 | NM_004706.3 | NM_004706 | Rho guanine nucleotide | ARHGEF1 | P115-RHOGEF; |
| | | | | | exchange factor (GEF) | | GEF1; LBCL2; |
| | | | | | 1 (ARHGEF1), | | SUB1.5 |
| | | | | | transcript variant 2, | | |
| | | | | | mRNA. | | |
| 499 | 474 | 870338 | NM_001964.2 | NM_001964 | early growth response | EGR1 | G0S30; AT225; TIS8; |
| | | | | | 1 (EGR1), mRNA. | | ZNF225; NGFI-A; |
| | | | | | | | KROX-24; ZIF-268 |
| 500 | 475 | 870408 | NM_000585.2 | NM_000585 | interleukin 15 (IL15), | IL15 | MGC9721; IL-15 |
| | | | | | transcript variant 3, | | |
| | | | | | mRNA. | | |
| 501 | 476 | 870500 | NM_015644.3 | NM_015644 | tubulin tyrosine ligase- | TTLL3 | MGC120532; |
| | | | | | like family, member 3 | | MGC120530; |
| | | | | | (TTLL3), transcript | | MGC120529; |
| | | | | | variant 2, mRNA. | | FLJ13898; HOTTL; |
| | | | | | | | DKFZp686D076; |
| | | | | | | | DKFZP434B103 |
| 502 | 477 | 870601 | NM_007027.2 | NM_007027 | topoisomerase (DNA) II | TOPBP1 | TOP2BP1 |
| | | | | | binding protein 1 | | |
| | | | | | (TOPBP1), mRNA. | | |
| 503 | 478 | 940020 | NM_020117.9 | NM_020117 | leucyl-tRNAsynthetase | LARS | PIG44; KIAA1352; |
| | | | | | (LARS), mRNA. | | hr025CI; FLJ10595; |
| | | | | | | | LEURS; LEUS; |
| | | | | | | | LARS1; LRS; |
| | | | | | | | FLJ21788; RNTLS; |
| | | | | | | | HSPC192 |
| 504 | 479 | 940725 | NM_172390.1 | NM_172390 | nuclear factor of | NFATC1 | NFATc; MGC138448; |
| | | | | | activated T-cells, | | NFAT2; NF-ATC |
| | | | | | cytoplasmic, | | |
| | | | | | calcineurin-dependent | | |
| | | | | | 1 (NFATC1), transcript | | |
| | | | | | variant 1, mRNA. | | |
| 505 | 480 | 990647 | NR_003367.1 | NR_003367 | Pvt1 oncogene | PVT1 | MGC21751 |
| | | | | | homolog (mouse) | | |
| | | | | | (PVT1) on | | |
| | | | | | chromosome 8. | | |
| 506 | 481 | 1010487 | NM_006763.2 | NM_006763 | BTG family, member 2 | BTG2 | PC3; TIS21; |
| | | | | | (BTG2), mRNA. | | MGC126064; |
| | | | | | | | MGC126063 |
| 507 | 482 | 1010592 | NM_001001548.1 | NM_001001548 | CD36 molecule | CD36 | GPIV; FAT; GP3B; |
| | | | | | (thrombospondin | | CHDS7; SCARB3; |
| | | | | | receptor) (CD36), | | PASIV; GP4 |
| | | | | | transcript variant 1, | | |
| | | | | | mRNA. | | |
| 508 | 483 | 1010719 | NM_004111.4 | NM_004111 | flap structure-specific | FEN1 | RAD2; FEN-1; MF1 |
| | | | | | endonuclease 1 | | |
| | | | | | (FEN1), mRNA. | | |
| 509 | 484 | 1030053 | NM_198055.1 | NM_198055 | myeloid zinc finger 1 | MZF1 | MZF1B; ZSCAN6; |
| | | | | | (MZF1), transcript | | MZF-1; ZNF42; Zfp98 |
| | | | | | variant 2, mRNA. | | |
| 510 | 485 | 1030167 | NM_001017373.2 | NM_001017373 | sterile alpha motif | SAMD3 | MGC35163; |
| | | | | | domain containing 3 | | FLJ34563 |
| | | | | | (SAMD3), transcript | | |
| | | | | | variant 1, mRNA. | | |
| 511 | 486 | 1030239 | NM_014766.3 | NM_014766 | secemin 1 (SCRN1), | SCRN1 | SES1; KIAA0193 |
| | | | | | mRNA. | | |
| 512 | 487 | 1030427 | NM_001014838.1 | NM_001014838 | cutA divalent cation | CUTA | MGC111154; |
| | | | | | tolerance homolog (E. coli) | | C6orf82; ACHAP |
| | | | | | (CUTA), transcript | | |
| | | | | | variant 4, mRNA. | | |
| 513 | 488 | 1030471 | NM_013336.3 | NM_013336 | Sec61 alpha 1 subunit | SEC61A1 | HSEC61; SEC61A; |
| | | | | | (S. cerevisiae) | | SEC61 |
| | | | | | (SEC61A1), mRNA. | | |
| 514 | 489 | 1030743 | NM_000595.2 | NM_000595 | lymphotoxin alpha | LTA | LT; TNFSF1; TNFB |
| | | | | | (TNF superfamily, | | |
| | | | | | member 1) (LTA), | | |
| | | | | | mRNA. | | |
| 515 | 490 | 1050309 | NM_021958.2 | NM_021958 | H2.0-like homeobox | HLX | HB24; HLX1 |
| | | | | | (HLX), mRNA. | | |
| 516 | 491 | 1050360 | NM_002121.4 | NM_002121 | major histocompatibility | HLA-DPB1 | HLA-DP1B; DPB1; |
| | | | | | complex, class II, DP | | MHC DPB1 |
| | | | | | beta 1 (HLA-DPB1), | | |
| | | | | | mRNA. | | |
| 517 | 492 | 1050612 | NM_177530.1 | NM_177530 | sulfotransferase family, | SULT1A1 | MGC5163; |
| | | | | | cytosolic, 1A, phenol- | | MGC131921; |
| | | | | | preferring, member 1 | | TSPST1; STP; PST; |
| | | | | | (SULT1A1), transcript | | HAST1/HAST2; P- |
| | | | | | variant 3, mRNA. | | PST; ST1A3; STP1 |
| 518 | 493 | 1050681 | NM_020652.1 | NM_020652 | zinc finger protein 286A | ZNF286A | KIAA1874; |
| | | | | | (ZNF286A), mRNA. | | MGC156181; |
| | | | | | | | ZNF286; MGC149627 |
| 519 | 494 | 1070450 | NM_080591.1 | NM_080591 | prostaglandin- | PTGS1 | PCOX1; PHS1; |
| | | | | | endoperoxide synthase | | PGHS1; COX1; |
| | | | | | 1 (prostaglandin G/H | | PGHS-1; PGG/HS; |
| | | | | | synthase and | | PTGHS; COX3 |
| | | | | | cyclooxygenase) | | |
| | | | | | (PTGS1), transcript | | |
| | | | | | variant 2, mRNA. | | |
| 520 | 495 | 1070475 | NM_000975.2 | NM_000975 | ribosomal protein L11 | RPL11 | GIG34 |
| | | | | | (RPL11), mRNA | | |
| 521 | 496 | 1070639 | NM_001293.1 | NM_001293 | chloride channel, | CLNS1A | CLNS1B; CLCI; ICIn |
| | | | | | nucleotide-sensitive, 1A | | |
| | | | | | (CLNS1A), mRNA. | | |
| 522 | 497 | 1090167 | NM_002070.2 | NM_002070 | guanine nucleotide | GNAI2 | H_LUCA16.1; GIP; |
| | | | | | binding protein (G | | GNAI2B; |
| | | | | | protein), alpha inhibiting | | H_LUCA15.1 |
| | | | | | activity polypeptide 2 | | |
| | | | | | (GNAI2), mRNA. | | |
| 523 | 498 | 1090239 | NM_012426.3 | NM_012426 | splicing factor 3b, | SF3B3 | SAP130; KIAA0017; |
| | | | | | subunit 3, 130 kDa | | RSE1; STAF130; |
| | | | | | (SF3B3), mRNA | | SF3b130 |
| 524 | 499 | 1090692 | NM_022913.1 | NM_022913 | GC-rich promoter | GPBP1 | GPBP; |
| | | | | | binding protein 1 | | DKFZp761C169; |
| | | | | | (GPBP1), mRNA. | | MGC126339 |
| 525 | 500 | 1170072 | NM_023072.1 | NM_023072 | zinc finger, SWIM-type | ZSWIM4 | — |
| | | | | | containing 4 (ZSWIM4), | | |
| | | | | | mRNA. | | |
| 526 | 501 | 1190026 | NM_021822.1 | NM_021822 | apolipoprotein B mRNA | APOBEC3G | CEM15; FLJ12740; |
| | | | | | editing enzyme, | | bK150C2.7; ARP9; |
| | | | | | catalytic polypeptide- | | dJ494G10.1; |
| | | | | | like 3G (APOBEC3G), | | MDS019 |
| | | | | | mRNA. | | |
| 527 | 502 | 1190142 | NM_032048.2 | NM_032048 | elastin | EMILIN2 | FLJ33200; FOAP-10; |
| | | | | | microfibrilinterfacer 2 | | EMILIN-2 |
| | | | | | (EMILIN2), mRNA. | | |
| 528 | 503 | 1190246 | NM_032590.3 | NM_032590 | F-box and leucine-rich | FBXL10 | CXXC2; Fbl10; |
| | | | | | repeat protein 10 | | JHDM1B; PCCX2 |
| | | | | | (FBXL10), transcript | | |
| | | | | | variant 1, mRNA. | | |
| 529 | 504 | 1190288 | NM_144599.3 | NM_144599 | non imprinted in | NIPA1 | SPG6; FSP3; |
| | | | | | Prader-Willi/Angelman | | MGC35570; |
| | | | | | syndrome 1 (NIPA1), | | MGC102724 |
| | | | | | mRNA. | | |
| 530 | 505 | 1190367 | NM_003897.3 | NM_003897 | immediate early | IER3 | IEX-1L; PRG1; IEX1; |
| | | | | | response 3 (IER3), | | GLY96; DIF-2; IEX-1; |
| | | | | | mRNA. | | DIF2 |
| 531 | 506 | 1190626 | NM_006624.3 | NM_006624 | zinc finger, MYND | ZMYND11 | RP11-486H9.1; |
| | | | | | domain containing 11 | | BRAM1; |
| | | | | | (ZMYND11), transcript | | MGC111056; BS69 |
| | | | | | variant 1, mRNA. | | |
| 532 | 507 | 1190634 | NM_001079804.1 | NM_001079804 | glucosidase, alpha; | GAA | LYAG |
| | | | | | acid (Pompe disease, | | |
| | | | | | glycogen storage | | |
| | | | | | disease type II) (GAA), | | |
| | | | | | transcript variant 3, | | |
| | | | | | mRNA. | | |
| 533 | 509 | 1230044 | NM_003143.1 | NM_003143 | single-stranded DNA | SSBP1 | SSBP |
| | | | | | binding protein 1 | | |
| | | | | | (SSBP1), mRNA. | | |
| 534 | 511 | 1230441 | NM_001005271.1 | NM_001005271 | chromodomain helicase | CHD3 | Mi2-ALPHA; Mi-2a; |
| | | | | | DNA binding protein 3 | | ZFH |
| | | | | | (CHD3), transcript | | |
| | | | | | variant 3, mRNA. | | |
| 535 | 512 | 1230450 | NM_017868.3 | NM_017868 | tetratricopeptide repeat | TTC12 | FLJ20535; FLJ13859; |
| | | | | | domain 12 (TTC12), | | TPARM |
| | | | | | mRNA. | | |
| 536 | 513 | 1230575 | NM_013986.2 | NM_013986 | Ewing sarcoma | EWSR1 | EWS |
| | | | | | breakpoint region 1 | | |
| | | | | | (EWSR1), transcript | | |
| | | | | | variant EWS-b, mRNA. | | |
| 537 | 514 | 1230630 | NM_181671.1 | NM_181671 | phosphatidylinositol | PITPNC1 | RDGBB; RDGB- |
| | | | | | transfer protein, | | BETA; RDGBB1 |
| | | | | | cytoplasmic 1 | | |
| | | | | | (PITPNC1), transcript | | |
| | | | | | variant 2, mRNA. | | |
| 538 | 515 | 1230639 | NM_016146.3 | NM_016146 | trafficking protein | TRAPPC4 | SBDN; CGI-104; |
| | | | | | particle complex 4 | | HSPC172; TRS23; |
| | | | | | (TRAPPC4), mRNA. | | PTD009 |
| 539 | 516 | 1230673 | NM_020230.4 | NM_020230 | peter pan homolog | PPAN | MGC14226; SSF2; |
| | | | | | (Drosophila) (PPAN), | | SSF1; BXDC3; SSF; |
| | | | | | mRNA. | | MGC45852 |
| 540 | 517 | 1230767 | NM_006435.2 | NM_006435 | interferon induced | IFITM2 | 1-8D |
| | | | | | transmembrane protein | | |
| | | | | | 2 (1-8D) (IFITM2), | | |
| | | | | | mRNA. | | |
| 541 | 518 | 1240050 | NM_198267.1 | NM_198267 | inhibitor of growth | ING3 | FLJ20089; p47ING3; |
| | | | | | family, member 3 | | ING2; Eaf4 |
| | | | | | (ING3), transcript | | |
| | | | | | variant 3, mRNA. | | |
| 542 | 519 | 1240152 | NM_001928.2 | NM_001928 | complement factor D | CFD | DF; ADN; PFD |
| | | | | | (adipsin) (CFD), | | |
| | | | | | mRNA. | | |
| 543 | 520 | 1240440 | NM_006472.2 | NM_006472 | thioredoxin interacting | TXNIP | EST01027; VDUP1; |
| | | | | | protein (TXNIP), | | THIF; HHCPA78 |
| | | | | | mRNA. | | |
| 544 | 521 | 1240504 | NM_018433.3 | NM_018433 | jumonji domain | JMJD1A | TSGA; JHMD2A; |
| | | | | | containing 1A | | KIAA0742; |
| | | | | | (JMJD1A), mRNA. | | DKFZp686A24246; |
| | | | | | | | JMJD1; |
| | | | | | | | DKFZp686P07111 |
| 545 | 522 | 1240553 | NM_014454.1 | NM_014454 | sestrin 1 (SESN1), | SESN1 | SEST1; MGC138241; |
| | | | | | mRNA. | | MGC142129; PA26; |
| | | | | | | | RP11-787I22.1 |
| 546 | 523 | 1240750 | NM_006750.2 | NM_006750 | syntrophin, beta 2 | SNTB2 | EST25263; |
| | | | | | (dystrophin-associated | | D16S2531E; |
| | | | | | protein A1, 59 kDa, | | SNT2B2; SNT3; |
| | | | | | basic component 2) | | SNTL |
| | | | | | (SNTB2), transcript | | |
| | | | | | variant 1, mRNA. | | |
| 547 | 524 | 1260341 | NM_001560.2 | NM_001560 | interleukin 13 receptor, | IL13RA1 | IL-13Ra; NR4; |
| | | | | | alpha 1 (IL13RA1), | | CD213A1 |
| | | | | | mRNA. | | |
| 548 | 525 | 1260524 | NM_003156.2 | NM_003156 | stromal interaction | STIM1 | GOK; D11S4896E |
| | | | | | molecule 1 (STIM1), | | |
| | | | | | mRNA. | | |
| 549 | 526 | 1300044 | NR_001545.1 | NR_001545 | testis-specific transciipt, | TTTY15 | DKFZP434I143 |
| | | | | | Y-linked 15 (TTTY15) | | |
| | | | | | on chromosome Y. | | |
| 550 | 527 | 1300228 | NM_017949.1 | NM_017949 | CUE domain containing | CUEDC1 | DKFZp547L163; |
| | | | | | 1 (CUEDC1), mRNA. | | FLJ20739 |
| 551 | 528 | 1300491 | NM_017443.3 | NM_017443 | polymerase (DNA | POLE3 | YBL1; p17; |
| | | | | | directed), epsilon 3 | | CHRAC17; |
| | | | | | (p17 subunit) (POLE3), | | CHARAC17 |
| | | | | | mRNA. | | |
| 552 | 529 | 1340291 | NM_145012.3 | NM_145012 | cyclin Y (CCNY), | CCNY | CBCP1; CFP1; |
| | | | | | transcript variant 1, | | C10orf9 |
| | | | | | mRNA. | | |
| 553 | 530 | 1340538 | NM_000199.2 | NM_000199 | N- | SGSH | HSS; MPS3A; SFMD |
| | | | | | sulfoglucosaminesulfohydrolase | | |
| | | | | | (sulfamidase) | | |
| | | | | | (SGSH), mRNA | | |
| 554 | 531 | 1400240 | NM_002300.4 | NM_002300 | lactate dehydrogenase | LDHB | LDH-H; TRG-5 |
| | | | | | B (LDHB), mRNA. | | |
| 555 | 532 | 1400446 | NM_001122.2 | NM_001122 | adipose differentiation- | ADFP | ADRP; MGC10598 |
| | | | | | related protein (ADFP), | | |
| | | | | | mRNA. | | |
| 556 | 533 | 1400703 | NM_019029.2 | NM_019029 | carboxypeptidase, | CPVL | HVLP; MGC10029 |
| | | | | | vitellogenic-like (CPVL), | | |
| | | | | | transcript variant 2, | | |
| | | | | | mRNA. | | |
| 557 | 534 | 1410161 | NM_001007075.1 | NM_001007075 | kelch-like 5 | KLHL5 | — |
| | | | | | (Drosophila) (KLHL5), | | |
| | | | | | transcript variant 2, | | |
| | | | | | mRNA. | | |
| 558 | 535 | 1410543 | NM_130469.2 | NM_130469 | Jun dimerization protein | JDP2 | JUNDM2 |
| | | | | | 2 (JDP2), mRNA. | | |
| 559 | 536 | 1410600 | NM_001007278.1 | NM_001007278 | tripartite motif- | TRIM13 | RNF77; CAR; LEU5; |
| | | | | | containing 13 | | RFP2; DLEU5 |
| | | | | | (TRIM13), transcript | | |
| | | | | | variant 4, mRNA. | | |
| 560 | 537 | 1430187 | NM_000100.2 | NM_000100 | cystatin B (stefin B) | CSTB | PME; CST6; EPM1; |
| | | | | | (CSTB), mRNA | | STFB |
| 561 | 538 | 1430280 | NM_004364.2 | NM_004364 | CCAAT/enhancer | CEBPA | CEBP; C/EBP-alpha |
| | | | | | binding protein | | |
| | | | | | (C/EBP), alpha | | |
| | | | | | (CEBPA), mRNA. | | |
| 562 | 539 | 1430360 | NM_015407.3 | NM_015407 | abhydrolase domain | ABHD14A | DORZ1; |
| | | | | | containing 14A | | DKFZP564O243 |
| | | | | | (ABHD14A), mRNA | | |
| 563 | 540 | 1440114 | NM_145902.1 | NM_145902 | high mobility group AT- | HMGA1 | MGC12816; |
| | | | | | hook 1 (HMGA1), | | MGC4854; HMG-R; |
| | | | | | transcript variant 4, | | MGC4242; HMGIY |
| | | | | | mRNA. | | |
| 564 | 541 | 1440187 | NM_016086.2 | NM_016086 | serine/threonine/tyrosine | STYXL1 | MK-STYX; DUSP24 |
| | | | | | interacting-like 1 | | |
| | | | | | (STYXL1), mRNA. | | |
| 565 | 542 | 1440243 | NM_016332.2 | NM_016332 | selenoprotein X, 1 | SEPX1 | MSRB1; SELR; |
| | | | | | (SEPX1), mRNA. | | MGC3344; HSPC270; |
| | | | | | | | SELX |
| 566 | 543 | 1440300 | NM_024330.1 | NM_024330 | solute carrier family 27 | SLC27A3 | VLCS-3; ACSVL3; |
| | | | | | (fatty acid transporter), | | MGC4365; FATP3 |
| | | | | | member 3 (SLC27A3), | | |
| | | | | | mRNA. | | |
| 567 | 544 | 1440605 | NM_033204.2 | NM_033204 | zinc finger protein 101 | ZNF101 | DKFZp570I0164; |
| | | | | | (ZNF101), mRNA. | | MGC149565; HZF12; |
| | | | | | | | MGC149566 |
| 568 | 545 | 1440612 | NM_014038.1 | NM_014038 | basic leucine zipper | BZW2 | MST017; MSTP017; |
| | | | | | and W2 domains 2 | | HSPC028 |
| | | | | | (BZW2), mRNA. | | |
| 569 | 546 | 1440750 | NM_030760.3 | NM_030760 | endothelial | EDG8 | SPPR-2; SPPR-1; |
| | | | | | differentiation, | | Edg-8; S1P5; S1PR5 |
| | | | | | sphingolipid G-protein- | | |
| | | | | | coupled receptor, 8 | | |
| | | | | | (EDG8), mRNA. | | |
| 570 | 547 | 1450273 | NM_024793.1 | NM_024793 | clusterin associated | CLUAP1 | FLJ13297; KIAA0643 |
| | | | | | protein 1 (CLUAP1), | | |
| | | | | | transcript variant 2, | | |
| | | | | | mRNA. | | |
| 571 | 548 | 1450707 | NM_002931.3 | NM_002931 | ring finger protein 1 | RING1 | RNF1 |
| | | | | | (RING1), mRNA | | |
| 572 | 549 | 1470386 | NM_153634.2 | NM_153634 | copine VIII (CPNE8), | CPNE8 | MGC129646; |
| | | | | | mRNA. | | MGC129645 |
| 573 | 550 | 1500152 | NM_007099.2 | NM_007099 | acid phosphatase 1, | ACP1 | HAAP; MGC3499; |
| | | | | | soluble (ACP1), | | MGC111030 |
| | | | | | transcript variant 2, | | |
| | | | | | mRNA. | | |
| 574 | 551 | 1500164 | NM_006196.2 | NM_006196 | poly(rC) binding protein | PCBP1 | hnRNP-E1; HNRPX; |
| | | | | | 1 (PCBP1), mRNA. | | HNRPE1; hnRNP-X |
| 575 | 552 | 1500470 | NM_030973.2 | NM_030973 | mediator complex | MED25 | TCBAP0758; ACID1; |
| | | | | | subunit 25 (MED25), | | MGC70671; ARC92; |
| | | | | | mRNA. | | DKFZp434K0512; |
| | | | | | | | P78 |
| 576 | 553 | 1500600 | NM_175738.3 | NM_175738 | RAB37, member RAS | RAB37 | FLJ32507; FLJ30284 |
| | | | | | oncogene family | | |
| | | | | | (RAB37), transcript | | |
| | | | | | variant 3, mRNA. | | |
| 577 | 554 | 1500711 | NM_014876.3 | NM_014876 | Josephin domain | JOSD1 | dJ508I15.2; |
| | | | | | containing 1 (JOSD1), | | KIAA0063 |
| | | | | | mRNA. | | |
| 578 | 555 | 1510026 | NM_017791.1 | NM_017791 | feline leukemia virus | FLVCR2 | FLVCR2; C14orf58; |
| | | | | | subgroup C cellular | | FLJ20371; CCT |
| | | | | | receptor family, | | |
| | | | | | member 2 (FLVCR2), | | |
| | | | | | mRNA. | | |
| 579 | 556 | 1510088 | XM_001133534.1 | XM_001133534 | PREDICTED: ATPase, | ATP1B3 | — |
| | | | | | Na+/K+ transporting, | | |
| | | | | | beta 3 polypeptide, | | |
| | | | | | transcript variant 2 | | |
| | | | | | (ATP1B3), mRNA. | | |
| 580 | 557 | 1510296 | NM_133436.1 | NM_133436 | asparagine synthetase | ASNS | TS11 |
| | | | | | (ASNS), transcript | | |
| | | | | | variant 1, mRNA. | | |
| 581 | 558 | 1510438 | NM_012087.2 | NM_012087 | general transcription | GTF3C5 | TFIIIC63; FLJ20857; |
| | | | | | factor IIIC, polypeptide | | TFIIICepsilon; TFiiiC2- |
| | | | | | 5, 63 kDa (GTF3C5), | | 63 |
| | | | | | mRNA. | | |
| 582 | 559 | 1510521 | NM_002573.2 | NM_002573 | platelet-activating factor | PAFAH1B3 | — |
| | | | | | acetylhydrolase, | | |
| | | | | | isoform lb, gamma | | |
| | | | | | subunit 29 kDa | | |
| | | | | | (PAFAH1B3), mRNA. | | |
| 583 | 560 | 1510630 | NM_017803.3 | NM_017803 | dihydrouridine synthase | DUS2L | SMM1; FLJ20399; |
| | | | | | 2-like, SMM1 homolog | | URLC8; DUS2 |
| | | | | | (S. cerevisiae) | | |
| | | | | | (DUS2L), mRNA. | | |
| 584 | 561 | 1510722 | NM_018434.4 | NM_018434 | ring finger protein 130 | RNF130 | G1RZFP; |
| | | | | | (RNF130), mRNA | | MGC138647; |
| | | | | | | | GOLIATH; |
| | | | | | | | MGC99542; |
| | | | | | | | MGC117241; GP |
| 585 | 562 | 1570338 | NM_198480.2 | NM_198480 | zinc finger protein 615 | ZNF615 | DKFZp686O1554; |
| | | | | | (ZNF615), mRNA. | | FLJ39372; FLJ33710 |
| 586 | 563 | 1570348 | NM_001759.2 | NM_001759 | cyclin D2 (CCND2), | CCND2 | KIAK0002; |
| | | | | | mRNA. | | MGC102758 |
| 587 | 564 | 1570725 | NM_144772.1 | NM_144772 | apolipoprotein A-I | APOA1BP | MGC119145; |
| | | | | | binding protein | | MGC119143; AIBP |
| | | | | | (APOA1BP), mRNA | | |
| 588 | 565 | 1580093 | NM_020680.3 | NM_020680 | SCY1-like 1 (S. cerevisiae) | SCYL1 | TRAP; P105; NTKL; |
| | | | | | (SCYL1), | | TEIF; GKLP; |
| | | | | | transcript variant A, | | MGC78454; NKTL; |
| | | | | | mRNA. | | TAPK; HT019 |
| 589 | 566 | 1580224 | NM_012241.2 | NM_012241 | sirtuin (silent mating | SIRT5 | SIR2L5 |
| | | | | | type information | | |
| | | | | | regulation 2 homolog) 5 | | |
| | | | | | (S. cerevisiae) (SIRT5), | | |
| | | | | | transcript variant 1, | | |
| | | | | | mRNA. | | |
| 590 | 567 | 1580309 | NM_001018108.2 | NM_001018108 | small EDRK-rich factor | SERF2 | 4F5REL; H4F5rel; |
| | | | | | 2 (SERF2), mRNA. | | FLJ37527; FLJ38557; |
| | | | | | | | FAM2C; MGC48826; |
| | | | | | | | HsT17089; FLJ20431 |
| 591 | 568 | 1580601 | NM_005800.3 | NM_005800 | ubiquitin specific | USPL1 | D135106E; C13orf22; |
| | | | | | peptidase like 1 | | DKFZp781K2286; |
| | | | | | (USPL1), mRNA. | | RP11-121O19.1; |
| | | | | | | | bA121O19.1; |
| | | | | | | | FLJ32952 |
| 592 | 569 | 1580719 | NM_022065.4 | NM_022065 | thyroid adenoma | THADA | GITA; FLJ44016; |
| | | | | | associated (THADA), | | KIAA1767; FLJ44876; |
| | | | | | transcript variant 1, | | FLJ21877; FLJ77530 |
| | | | | | mRNA. | | |
| 593 | 571 | 1660113 | NM_001079673.1 | NM_001079673 | fibronectin type III | FNDC3A | KIAA0970; |
| | | | | | domain containing 3A | | bA203I16.5; FNDC3; |
| | | | | | (FNDC3A), transcript | | RP11-203I16.5; |
| | | | | | variant 1, mRNA. | | bA203I16.1; |
| | | | | | | | FLJ31509 |
| 594 | 572 | 1660215 | NM_005177.3 | NM_005177 | ATPase, H+ | ATP6V0A1 | ATP6N1; VPP1; |
| | | | | | transporting, lysosomal | | DKFZp781J1951; |
| | | | | | V0 subunit a1 | | Stv1; a1; ATP6N1A; |
| | | | | | (ATP6V0A1), mRNA. | | Vph1 |
| 595 | 573 | 1660278 | NM_012458.2 | NM_012458 | translocase of inner | TIMM13 | ppv1; TIMM13B; |
| | | | | | mitochondrial | | TIM13B; TIM13; |
| | | | | | membrane 13 homolog | | TIMM13A |
| | | | | | (yeast) (TIMM13), | | |
| | | | | | nuclear gene encoding | | |
| | | | | | mitochondrial protein, | | |
| | | | | | mRNA. | | |
| 596 | 574 | 1660397 | NM_003153.3 | NM_003153 | signal transducer and | STAT6 | STAT6C; D1251644; |
| | | | | | activator of transcription | | STAT6B; IL-4-STAT |
| | | | | | 6, interleukin-4 induced | | |
| | | | | | (STAT6), mRNA. | | |
| 597 | 575 | 1660646 | NM_018151.3 | NM_018151 | RAP1 interacting factor | RIF1 | DKFZp781N1478; |
| | | | | | homolog (yeast) (RIF1), | | FLJ12870 |
| | | | | | mRNA. | | |
| 598 | 576 | 1660685 | NM_018460.2 | NM_018460 | Rho GTPase activating | ARHGAP15 | BM046 |
| | | | | | protein 15 | | |
| | | | | | (ARHGAP15), mRNA. | | |
| 599 | 577 | 1690209 | NM_203385.1 | NM_203385 | ribonuclease/angiogenin | RNH1 | MGC54054; RAI; |
| | | | | | inhibitor 1 (RNH1), | | MGC4569; |
| | | | | | transcript variant 4, | | MGC18200; RNH |
| | | | | | mRNA. | | |
| 600 | 578 | 1690288 | NM_006901.1 | NM_006901 | myosin IXA (MYO9A), | MYO9A | MGC71859; |
| | | | | | mRNA. | | FLJ13244; FLJ11061 |
| 601 | 579 | 1710070 | NM_000632.3 | NM_000632 | integdn, alpha M | ITGAM | MO1A; MAC1A; |
| | | | | | (complement | | CD11B; MAC-1; |
| | | | | | component 3 receptor 3 | | CR3A; MGC117044 |
| | | | | | subunit) (ITGAM), | | |
| | | | | | mRNA. | | |
| 602 | 580 | 1710369 | NM_001033853.1 | NM_001033853 | ribosomal protein L3 | RPL3 | MGC104284; |
| | | | | | (RPL3), transcript | | TARBP-B |
| | | | | | variant 2, mRNA. | | |
| 603 | 581 | 1710541 | NM_144649.1 | NM_144649 | transmembrane protein | TMEM71 | FLJ33069; |
| | | | | | 71 (TMEM71), mRNA. | | MGC111188 |
| 604 | 582 | 1710630 | NM_003032.2 | NM_003032 | ST6 beta- | ST6GAL1 | ST6Gal I; SIAT1; |
| | | | | | galactosamide alpha- | | ST6GalI; MGC48859; |
| | | | | | 2,6-sialyltranferase 1 | | CD75 |
| | | | | | (ST6GAL1), transcript | | |
| | | | | | variant 2, mRNA. | | |
| 605 | 583 | 1740050 | XM_495863.3 | XM_495863 | PREDICTED: GTPase, | GVIN1 | — |
| | | | | | very large interferon | | |
| | | | | | inducible 1 (GVIN1), | | |
| | | | | | mRNA. | | |
| 606 | 584 | 1740136 | NM_018976.3 | NM_018976 | solute carrier family 38, | SLC38A2 | PRO1068; SAT2; |
| | | | | | member 2 (SLC38A2), | | SNAT2; KIAA1382; |
| | | | | | mRNA. | | ATA2 |
| 607 | 585 | 1740373 | NM_003565.1 | NM_003565 | unc-51-like kinase 1 (C. elegans) | ULK1 | FLJ38455; UNC51; |
| | | | | | (ULK1), | | Unc51.1; ATG1 |
| | | | | | mRNA. | | |
| 608 | 586 | 1740471 | NM_012461.1 | NM_012461 | TERF1 (TRF1)- | TINF2 | TIN2 |
| | | | | | interacting nuclear | | |
| | | | | | factor 2 (TINF2), | | |
| | | | | | mRNA. | | |
| 609 | 587 | 1740646 | NM_003737.2 | NM_003737 | dachsous 1 | DCHS1 | CDH25; KIAA1773; |
| | | | | | (Drosophila) (DCHS1), | | PCDH16; FLJ11790; |
| | | | | | mRNA. | | FIB1 |
| 610 | 588 | 1770035 | NM_017851.4 | NM_017851 | poly (ADP-ribose) | PARP16 | FLJ25281; FLJ20509; |
| | | | | | polymerase family, | | C15orf30 |
| | | | | | member 16 (PARP16), | | |
| | | | | | mRNA. | | |
| 611 | 589 | 1770152 | NM_152851.1 | NM_152851 | membrane-spanning 4- | MS4A6A | CDA01; MSTP090; |
| | | | | | domains, subfamily A, | | MGC22650; 4SPAN3; |
| | | | | | member 6A (MS4A6A), | | 4SPAN3.2; MS4A6; |
| | | | | | transcript variant 3, | | CD20L3; MST090; |
| | | | | | mRNA. | | MGC131944 |
| 612 | 590 | 1770273 | NM_001029862.1 | NM_001029862 | ankyrin repeat domain | ANKRD30B | NY-BR-1.1 |
| | | | | | 30B (ANKRD30B), | | |
| | | | | | mRNA. | | |
| 613 | 591 | 1770546 | NM_004044.4 | NM_004044 | 5-aminoimidazole-4- | ATIC | PURH; IMPCHASE; |
| | | | | | carboxamide | | AICARFT; AICAR |
| | | | | | ribonucleotide | | |
| | | | | | formyltransferase/IMP | | |
| | | | | | cyclohydrolase (ATIC), | | |
| | | | | | mRNA. | | |
| 614 | 592 | 1770717 | NM_003609.2 | NM_003609 | HIRA interacting protein | HIRIP3 | — |
| | | | | | 3 (HIRIP3), mRNA. | | |
| 615 | 593 | 1770730 | NM_003971.3 | NM_003971 | sperm associated | SPAG9 | FLJ34602; |
| | | | | | antigen 9 (SPAG9), | | MGC14967; |
| | | | | | mRNA. | | MGC117291; |
| | | | | | | | FLJ26141; JLP; |
| | | | | | | | HLC4; FLJ13450; |
| | | | | | | | PHET; MGC74461; |
| | | | | | | | HSS; PIG6; |
| | | | | | | | FLJ14006; KIAA0516 |
| 616 | 594 | 1780259 | NM_017918.3 | NM_017918 | coiled-coil domain | CCDC109B | FLJ20647 |
| | | | | | containing 109B | | |
| | | | | | (CCDC109B), mRNA. | | |
| 617 | 595 | 1780411 | NM_148172.1 | NM_148172 | phosphatidylethanolamine | PEMT | PEAMT; MGC2483; |
| | | | | | N-methyltransferase | | PEMPT; PNMT; |
| | | | | | (PEMT), nuclear gene | | PEMT2 |
| | | | | | encoding mitochondrial | | |
| | | | | | protein, transcript | | |
| | | | | | variant 1, mRNA. | | |
| 618 | 596 | 1780730 | NM_014167.2 | NM_014167 | coiled-coil domain | CCDC59 | FLJ10294; HSPC128 |
| | | | | | containing 59 | | |
| | | | | | (CCDC59), mRNA. | | |
| 619 | 597 | 1820037 | NM_000617.1 | NM_000617 | solute carrier family 11 | SLC11A2 | FLJ37416; NRAMP2; |
| | | | | | (proton-coupled | | DCT1; DMT1 |
| | | | | | divalent metal ion | | |
| | | | | | transporters), member | | |
| | | | | | 2 (SLC11A2), mRNA. | | |
| 620 | 598 | 1820053 | NM_152346.1 | NM_152346 | solute carrier family 43, | SLC43A2 | FLJ23848; LAT4; |
| | | | | | member 2 (SLC43A2), | | MGC34680 |
| | | | | | mRNA. | | |
| 621 | 599 | 1820279 | NM_130783.3 | NM_130783 | tetraspanin 18 | TSPAN18 | TSPAN |
| | | | | | (TSPAN18), transcript | | |
| | | | | | variant 2, mRNA. | | |
| 622 | 600 | 1820300 | NM_001334.2 | NM_001334 | cathepsin O (CTSO), | CTSO | CTSO1 |
| | | | | | mRNA. | | |
| 623 | 601 | 1820379 | NM_000954.5 | NM_000954 | prostaglandin D2 | PTGDS | PGD2; PGDS; |
| | | | | | synthase 21 kDa (brain) | | PGDS2; PDS |
| | | | | | (PTGDS), mRNA. | | |
| 624 | 602 | 1820438 | NM_001014380.1 | NM_001014380 | katanin p60 subunit A- | KATNAL1 | MGC2599 |
| | | | | | like 1 (KATNAL1), | | |
| | | | | | transcript variant 2, | | |
| | | | | | mRNA. | | |
| 625 | 603 | 1820441 | NM_144567.3 | NM_144567 | angel homolog 2 | ANGEL2 | FLJ12793; |
| | | | | | (Drosophila) | | KIAA0759L |
| | | | | | (ANGEL2), mRNA. | | |
| 626 | 604 | 1820470 | NM_016167.3 | NM_016167 | nucleolar protein 7, | NOL7 | RARG-1; dJ223E5.2; |
| | | | | | 27 kDa (NOL7), mRNA. | | MGC71933; C6orf90 |
| 627 | 605 | 1820504 | NM_000269.2 | NM_000269 | non-metastatic cells 1, | NME1 | NDPKA; NM23-H1; |
| | | | | | protein (NM23A) | | NDPK-A; NM23; |
| | | | | | expressed in (NME1), | | AWD; GAAD |
| | | | | | transcript variant 2, | | |
| | | | | | mRNA. | | |
| 628 | 606 | 1820543 | NM_182498.2 | NM_182498 | zinc finger protein 428 | ZNF428 | MGC51082; Zfp428; |
| | | | | | (ZNF428), mRNA. | | C19orf37 |
| 629 | 607 | 1850259 | NM_205843.1 | NM_205843 | nuclear factor I/C | NFIC | CTF5; CTF; NF-I; NFI; |
| | | | | | (CCAAT-binding | | MGC20153 |
| | | | | | transcription factor) | | |
| | | | | | (NFIC), transcript | | |
| | | | | | variant 2, mRNA. | | |
| 630 | 608 | 1850338 | NM_001000.2 | NM_001000 | ribosomal protein L39 | RPL39 | — |
| | | | | | (RPL39), mRNA. | | |
| 631 | 609 | 1850523 | NM_004131.3 | NM_004131 | granzyme B (granzyme | GZMB | CCPI; CGL1; |
| | | | | | 2, cytotoxic T- | | CTSGL1; CGL-1; |
| | | | | | lymphocyte-associated | | CSP-B; CTLA1; |
| | | | | | serine esterase 1) | | CSPB; SECT; HLP |
| | | | | | (GZMB), mRNA. | | |
| 632 | 610 | 1850612 | NM_005484.2 | NM_005484 | poly (ADP-ribose) | PARP2 | ADPRTL3; ADPRTL2; |
| | | | | | polymerase family, | | PARP-2; ADPRT2; |
| | | | | | member 2 (PARP2), | | pADPRT-2 |
| | | | | | mRNA. | | |
| 633 | 611 | 1940021 | NM_002087.2 | NM_002087 | granulin (GRN), mRNA. | GRN | PEPI; PGRN; |
| | | | | | | | PCDGF; GEP; GP88 |
| 634 | 612 | 1940129 | NM_001040023.1 | NM_001040023 | signal-regulatory | SIRPA | SIRPalpha2; BIT; |
| | | | | | protein alpha (SIRPA), | | SHPS1; MFR; SIRP; |
| | | | | | transcript variant 2, | | SHPS-1; CD172A; |
| | | | | | mRNA. | | SIRP-ALPHA-1; |
| | | | | | | | SIRPalpha; PTPNS1; |
| | | | | | | | P84; MYD-1 |
| 635 | 613 | 1940187 | NM_130797.2 | NM_130797 | dipeptidyl-peptidase 6 | DPP6 | DPPX; MGC46605 |
| | | | | | (DPP6), transcript | | |
| | | | | | variant 1, mRNA. | | |
| 636 | 614 | 1940193 | NM_022055.1 | NM_022055 | potassium channel, | KCNK12 | THIK2; THIK-2 |
| | | | | | subfamily K, member | | |
| | | | | | 12 (KCNK12), mRNA. | | |
| 637 | 615 | 1940402 | NM_001013706.2 | NM_001013706 | lipid storage droplet | LSDP5 | — |
| | | | | | protein 5 (LSDP5), | | |
| | | | | | mRNA. | | |
| 638 | 616 | 1980059 | NM_170774.1 | NM_170774 | Ras association | RASSF2 | DKFZp781O1747; |
| | | | | | (RalGDS/AF-6) domain | | KIAA0168 |
| | | | | | family member 2 | | |
| | | | | | (RASSF2), transcript | | |
| | | | | | variant 2, mRNA. | | |
| 639 | 617 | 1980220 | NM_001039712.1 | NM_001039712 | death effector domain | DEDD | KE05; CASP8IP1; |
| | | | | | containing (DEDD), | | FLDED1; DEDD1; |
| | | | | | transcript variant 4, | | DEFT |
| | | | | | mRNA. | | |
| 640 | 618 | 1980242 | NM_001239.2 | NM_001239 | cyclin H (CCNH), | CCNH | p34; CAK; p37 |
| | | | | | mRNA. | | |
| 641 | 619 | 1980553 | NM_003815.3 | NM_003815 | ADAM | ADAM15 | MDC15 |
| | | | | | metallopeptidase | | |
| | | | | | domain 15 (ADAM15), | | |
| | | | | | transcript variant 2, | | |
| | | | | | mRNA. | | |
| 642 | 620 | 1980632 | NM_001539.2 | NM_001539 | DnaJ (Hsp40) homolog, | DNAJA1 | DjA1; hDJ-2; HSJ2; |
| | | | | | subfamily A, member 1 | | DJ-2; HSPF4; HSDJ; |
| | | | | | (DNAJA1), mRNA. | | HDJ2 |
| 643 | 621 | 1980762 | NM_133491.2 | NM_133491 | spermidine/spermine | SAT2 | SSAT2 |
| | | | | | N1-acetyltransferase | | |
| | | | | | family member 2 | | |
| | | | | | (SAT2), mRNA. | | |
| 644 | 622 | 1990379 | NM_152270.2 | NM_152270 | schlafen family member | SLFN11 | FLJ34922; SLFN8/9 |
| | | | | | 11 (SLFN11), mRNA. | | |
| 645 | 623 | 1990397 | NM_004510.2 | NM_004510 | SP110 nuclear body | SP110 | FLJ22835; IFI75; |
| | | | | | protein (SP110), | | VODI; IFI41 |
| | | | | | transcript variant b, | | |
| | | | | | mRNA. | | |
| 646 | 624 | 2000167 | NM_032876.4 | NM_032876 | jub, ajuba homolog | JUB | Ajuba; MGC15563 |
| | | | | | (Xenopus laevis) (JUB), | | |
| | | | | | transcript variant 1, | | |
| | | | | | mRNA. | | |
| 647 | 625 | 2000195 | NM_017599.2 | NM_017599 | vezatin, adherens | VEZT | DKFZp761C241; |
| | | | | | junctions | | VEZATIN |
| | | | | | transmembrane protein | | |
| | | | | | (VEZT), mRNA. | | |
| 648 | 626 | 2000482 | NM_000107.1 | NM_000107 | damage-specific DNA | DDB2 | FLJ34321 |
| | | | | | binding protein 2, | | |
| | | | | | 48 kDa (DDB2), mRNA. | | |
| 649 | 627 | 2000500 | NM_016047.3 | NM_016047 | splicing factor 3B, 14 kDa | SF3B14 | Ht006; SF3B14a; |
| | | | | | subunit (SF3B14), | | SAP14; CGI-110; |
| | | | | | mRNA. | | HSPC175; P14 |
| 650 | 628 | 2030088 | NM_178273.1 | NM_178273 | paired immunoglobin- | PILRA | FDF03 |
| | | | | | like type 2 receptor | | |
| | | | | | alpha (PILRA), | | |
| | | | | | transcript variant 3, | | |
| | | | | | mRNA. | | |
| 651 | 629 | 2030360 | NM_001042693.1 | NM_001042693 | hypothetical protein | MGC52498 | PRO7171; |
| | | | | | MGC52498 | | WWLS2783 |
| | | | | | (MGC52498), mRNA. | | |
| 652 | 630 | 2060047 | NM_004776.2 | NM_004776 | UDP-Gal:betaGlcNAc | B4GALT5 | beta4Gal-T5; BETA4- |
| | | | | | beta 1,4- | | GALT-IV; B4Gal-T5; |
| | | | | | galactosyltransferase, | | gt-V; beta4GalT-V |
| | | | | | polypeptide 5 | | |
| | | | | | (B4GALT5), mRNA. | | |
| 653 | 631 | 2060121 | NM_000147.3 | NM_000147 | fucosidase, alpha-L-1, | FUCA1 | — |
| | | | | | tissue (FUCA1), | | |
| | | | | | mRNA. | | |
| 654 | 632 | 2060154 | NM_024742.1 | NM_024742 | armadillo repeat | ARMC5 | FLJ00019; FLJ13063 |
| | | | | | containing 5 (ARMC5), | | |
| | | | | | mRNA. | | |
| 655 | 633 | 2060286 | NM_005134.2 | NM_005134 | protein phosphatase 4, | PPP4R1 | PP4R1; PP4(Rmeg) |
| | | | | | regulatory subunit 1 | | |
| | | | | | (PPP4R1), transcript | | |
| | | | | | variant 2, mRNA. | | |
| 656 | 634 | 2070037 | NM_012092.2 | NM_012092 | inducible T-cell co- | ICOS | CD278; MGC39850; |
| | | | | | stimulator (ICOS), | | AILIM |
| | | | | | mRNA. | | |
| 657 | 635 | 2070088 | NM_002127.3 | NM_002127 | HLA-G | HLA-G | MHC-G |
| | | | | | histocompatibility | | |
| | | | | | antigen, class I, G | | |
| | | | | | (HLA-G), mRNA. | | |
| 658 | 636 | 2070291 | NM_002633.2 | NM_002633 | phosphoglucomutase 1 | PGM1 | — |
| | | | | | (PGM1), mRNA. | | |
| 659 | 637 | 2070376 | XM_001132711.1 | XM_001132711 | PREDICTED: radical | RFNG | — |
| | | | | | fringe homolog | | |
| | | | | | (Drosophila) (RFNG), | | |
| | | | | | mRNA. | | |
| 660 | 638 | 2070736 | NM_138341.1 | NM_138341 | transmembrane protein | TMEM116 | FLJ90167 |
| | | | | | 116 (TMEM116), | | |
| | | | | | mRNA. | | |
| 661 | 639 | 2100139 | NM_005455.3 | NM_005455 | zinc finger, RAN- | ZRANB2 | DKFZp686J1831; |
| | | | | | binding domain | | DKFZp686N09117; |
| | | | | | containing 2 (ZRANB2), | | ZIS2; ZIS1; |
| | | | | | transcript variant 2, | | FLJ41119; ZIS; |
| | | | | | mRNA. | | ZNF265 |
| 662 | 640 | 2120053 | NM_000104.2 | NM_000104 | cytochrome P450, | CYP1B1 | GLC3A; CP1B |
| | | | | | family 1, subfamily B, | | |
| | | | | | polypeptide 1 | | |
| | | | | | (CYP1B1), mRNA. | | |
| 663 | 641 | 2120224 | NM_033121.1 | NM_033121 | ankyrin repeat domain | ANKRD13A | ANKRD13; NY-REN- |
| | | | | | 13A (ANKRD13A), | | 25 |
| | | | | | mRNA. | | |
| 664 | 642 | 2120360 | NM_006950.3 | NM_006950 | synapsin I (SYN1), | SYN1 | SYN1a; SYNI; SYN1b |
| | | | | | transcript variant Ia, | | |
| | | | | | mRNA. | | |
| 665 | 643 | 2120500 | NM_152233.2 | NM_152233 | sorting nexin 6 (SNX6), | SNX6 | MSTP010; MGC3157; |
| | | | | | transcript variant 2, | | TFAF2 |
| | | | | | mRNA. | | |
| 666 | 644 | 2140202 | NM_020711.1 | NM_020711 | ermin, ERM-like protein | ERMN | JN; KIAA1189; ermin |
| | | | | | (ERMN), transcript | | |
| | | | | | variant 2, mRNA. | | |
| 667 | 645 | 2140288 | NM_024829.4 | NM_024829 | hypothetical protein | FLJ22662 | — |
| | | | | | FLJ22662 (FLJ22662), | | |
| | | | | | mRNA. | | |
| 668 | 646 | 2140364 | NM_022340.2 | NM_022340 | zinc finger, FYVE | ZFYVE20 | MGC126210; |
| | | | | | domain containing 20 | | FLJ34993; |
| | | | | | (ZFYVE20), mRNA. | | Rabenosyn-5 |
| 669 | 647 | 2140382 | NM_015148.2 | NM_015148 | PAS domain containing | PASK | KIAA0135; STK37; |
| | | | | | serine/threonine kinase | | PASKIN; |
| | | | | | (PASK), mRNA | | DKFZp686P2031; |
| | | | | | | | DKFZP434O051 |
| 670 | 648 | 2140735 | NM_006266.2 | NM_006266 | ral guanine nucleotide | RALGDS | RGF; RalGEF; |
| | | | | | dissociation stimulator | | FLJ20922 |
| | | | | | (RALGDS), transcript | | |
| | | | | | variant 1, mRNA. | | |
| 671 | 649 | 2190064 | NM_003756.2 | NM_003756 | eukaryotic translation | EIF3H | EIF3S3; eIF3h; |
| | | | | | initiation factor 3, | | MGC102958; eIF3- |
| | | | | | subunit H (EIF3H), | | p40; eIF3-gamma |
| | | | | | mRNA. | | |
| 672 | 650 | 2190189 | NM_015934.3 | NM_015934 | nucleolar protein | NOP5/NOP58 | HSPC120 |
| | | | | | NOP5/NOP58 | | |
| | | | | | (NOP5/NOP58), | | |
| | | | | | mRNA. | | |
| 673 | 651 | 2190390 | NM_147223.2 | NM_147223 | nuclear receptor | NCOA1 | RIP160; NCoA-1; |
| | | | | | coactivator 1 (NCOA1), | | SRC1; MGC129719; |
| | | | | | transcript variant 2, | | MGC129720; F-SRC-1 |
| | | | | | mRNA. | | |
| 674 | 652 | 2190730 | NM_001007794.1 | NM_001007794 | choline/ethanolamine | CEPT1 | MGC45223; |
| | | | | | phosphotransferase 1 | | DKFZp313G0615 |
| | | | | | (CEPT1), transcript | | |
| | | | | | variant 2, mRNA. | | |
| 675 | 653 | 2230019 | NM_001078170.1 | NM_001078170 | RANBP2-like and GRIP | RGPD2 | RGP2; RANBP2L2; |
| | | | | | domain containing 2 | | NUP358 |
| | | | | | (RGPD2), mRNA. | | |
| | | | | | XM_001134112 | | |
| | | | | | XM_001134114 | | |
| | | | | | XM_001134116 | | |
| 676 | 654 | 2230408 | NM_006578.3 | NM_006578 | guanine nucleotide | GNB5 | FLJ37457; FLJ43714; |
| | | | | | binding protein (G | | GB5 |
| | | | | | protein), beta 5 | | |
| | | | | | (GNB5), transcript | | |
| | | | | | variant 1, mRNA. | | |
| 677 | 655 | 2230431 | NM_002200.3 | NM_002200 | interferon regulatory | IRF5 | — |
| | | | | | factor 5 (IRF5), | | |
| | | | | | transcript variant 1, | | |
| | | | | | mRNA. | | |
| 678 | 656 | 2230730 | NM_018079.3 | NM_018079 | S1 RNA binding | SRBD1 | FLJ10379 |
| | | | | | domain 1 (SRBD1), | | |
| | | | | | mRNA. | | |
| 679 | 657 | 2260129 | NM_022349.2 | NM_022349 | membrane-spanning 4- | MS4A6A | CDA01; MSTP090; |
| | | | | | domains, subfamily A, | | MGC22650; 4SPAN3; |
| | | | | | member 6A (MS4A6A), | | 4SPAN3.2; MS4A6; |
| | | | | | transcript variant 2, | | CD20L3; MST090; |
| | | | | | mRNA. | | MGC131944 |
| 680 | 659 | 2260619 | NM_080875.1 | NM_080875 | mindbomb homolog 2 | MIB2 | ZZZ5; ZZANK1; |
| | | | | | (Drosophila) (MIB2), | | FLJ20648; FLJ39787 |
| | | | | | mRNA. | | |
| 681 | 660 | 2320215 | NM_024923.2 | NM_024923 | nucleoporin 210 kDa | NUP210 | KIAA0906; FLJ22389; |
| | | | | | (NUP210), mRNA. | | GP210; POM210 |
| 682 | 661 | 2320286 | NM_004973.2 | NM_004973 | jumonji, AT rich | JARID2 | JMJ |
| | | | | | interactive domain 2 | | |
| | | | | | (JARID2), mRNA | | |
| 683 | 662 | 2320722 | NM_014957.2 | NM_014957 | DENN/MADD domain | DENND3 | KIAA0870 |
| | | | | | containing 3 | | |
| | | | | | (DENND3), mRNA. | | |
| 684 | 663 | 2340072 | NM_022750.2 | NM_022750 | poly (ADP-ribose) | PARP12 | MSTP109; FLJ22693; |
| | | | | | polymerase family, | | ZC3H1; ZC3HDC1; |
| | | | | | member 12 (PARP12), | | MST109; PARP-12 |
| | | | | | mRNA. | | |
| 685 | 664 | 2340180 | NM_003564.1 | NM_003564 | transgelin 2 (TAGLN2), | TAGLN2 | KIAA0120; HA1756 |
| | | | | | mRNA. | | |
| 686 | 665 | 2350215 | NM_021807.3 | NM_021807 | exocyst complex | EXOC4 | SEC8L1; REC8; |
| | | | | | component 4 (EXOC4), | | Sec8p; SEC8; |
| | | | | | transcript variant 1, | | MGC27170 |
| | | | | | mRNA. | | |
| 687 | 666 | 2350468 | NM_001077203.1 | NM_001077203 | SUMO1/sentrin specific | SENP7 | KIAA1707; |
| | | | | | peptidase 7 (SENP7), | | MGC157730 |
| | | | | | transcript variant 2, | | |
| | | | | | mRNA. | | |
| 688 | 667 | 2360326 | NM_003186.3 | NM_003186 | transgelin (TAGLN), | TAGLN | TAGLN1; WS3-10; |
| | | | | | transcript variant 2, | | SM22; |
| | | | | | mRNA. | | DKFZp686P11128; |
| | | | | | | | SMCC |
| 689 | 668 | 2360711 | NM_024010.1 | NM_024010 | 5- | MTRR | MSR; MGC129643 |
| | | | | | methyltetrahydrofolate- | | |
| | | | | | homocysteine | | |
| | | | | | methyltransferasereductase | | |
| | | | | | (MTRR), transcript | | |
| | | | | | variant 2, mRNA. | | |
| 690 | 669 | 2360719 | NM_007199.1 | NM_007199 | interleukin-1 receptor- | IRAK3 | IRAKM; ASRT5; |
| | | | | | associated kinase 3 | | IRAK-M |
| | | | | | (IRAK3), mRNA. | | |
| 691 | 671 | 2370064 | NM_001671.2 | NM_001671 | asialoglycoprotein | ASGR1 | ASGPR; Hs.12056; |
| | | | | | receptor 1 (ASGR1), | | CLEC4H1 |
| | | | | | mRNA. | | |
| 692 | 672 | 2370128 | NM_032839.1 | NM_032839 | disrupted in renal | DIRC2 | FLJ14784; RCC4 |
| | | | | | carcinoma 2 (DIRC2), | | |
| | | | | | mRNA. | | |
| 693 | 673 | 2370228 | NM_033124.2 | NM_033124 | coiled-coil domain | CCDC65 | FLJ25663; NYD- |
| | | | | | containing 65 | | SP28; FLJ35732 |
| | | | | | (CCDC65), mRNA. | | |
| 694 | 674 | 2450131 | NM_020338.2 | NM_020338 | zinc finger, MIZ-type | ZMIZ1 | MIZ; Zimp10; |
| | | | | | containing 1 (ZMIZ1), | | FLJ13541; hZIMP10; |
| | | | | | mRNA. | | KIAA1224; RAI17 |
| 695 | 675 | 2450132 | NM_030573.2 | NM_030573 | THAP domain | THAP7 | MGC10963 |
| | | | | | containing 7 (THAP7), | | |
| | | | | | transcript variant 1, | | |
| | | | | | mRNA. | | |
| 696 | 676 | 2450280 | NM_016505.2 | NM_016505 | zinc finger, CCHC | ZCCHC17 | pNO40; PS1D; |
| | | | | | domain containing 17 | | HSPC251; RP11- |
| | | | | | (ZCCHC17), mRNA. | | 266K22.1 |
| 697 | 677 | 2450427 | NM_005601.3 | NM_005601 | natural killer cell group | NKG7 | GIG1 |
| | | | | | 7 sequence (NKG7), | | |
| | | | | | mRNA. | | |
| 698 | 678 | 2450554 | NM_203284.1 | NM_203284 | recombination signal | RBPJ | SUH; RBP-J; RBPJK; |
| | | | | | binding protein for | | csl; KBF2; IGKJRB1; |
| | | | | | immunoglobulin kappa | | IGKJRB; CBF1; |
| | | | | | J region (RBPJ), | | RBPSUH; |
| | | | | | transcript variant 4, | | MGC61669 |
| | | | | | mRNA. | | |
| 699 | 679 | 2450563 | NM_001008738.2 | NM_001008738 | folliculin interacting | FNIP1 | DKFZp781P0215; |
| | | | | | protein 1 (FNIP1), | | KIAA1961; MGC667; |
| | | | | | transcript variant 2, | | DKFZp686E18167 |
| | | | | | mRNA. | | |
| 700 | 680 | 2450707 | NR_002825.1 | NR_002825 | sialic acid binding Ig- | SIGLECP16 | Siglec-P16 |
| | | | | | like lectin, pseudogene | | |
| | | | | | 16 (SIGLECP16) on | | |
| | | | | | chromosome 19. | | |
| 701 | 681 | 2450762 | NM_014002.2 | NM_014002 | inhibitor of kappa light | IKBKE | MGC125295; |
| | | | | | polypeptide gene | | MGC125297; IKKI; |
| | | | | | enhancer in B-cells, | | MGC125294; IKK-i; |
| | | | | | kinase epsilon (IKBKE), | | IKKE; KIAA0151 |
| | | | | | mRNA. | | |
| 702 | 682 | 2470070 | NM_005647.2 | NM_005647 | transducin (beta)-like | TBL1X | EBI; TBL1 |
| | | | | | 1X-linked (TBL1X), | | |
| | | | | | mRNA. | | |
| 703 | 683 | 2470079 | NM_001032293.2 | NM_001032293 | zinc finger protein 207 | ZNF207 | DKFZp761N202 |
| | | | | | (ZNF207), transcript | | |
| | | | | | variant 2, mRNA. | | |
| 704 | 684 | 2470097 | NM_037370.1 | NM_037370 | cyclin D-type binding- | CCNDBP1 | DIP1; GCIP |
| | | | | | protein 1 (CCNDBP1), | | |
| | | | | | transcript variant 2, | | |
| | | | | | mRNA. | | |
| 705 | 685 | 2470634 | NM_006195.4 | NM_006195 | pre-B-cell leukemia | PBX3 | — |
| | | | | | homeobox 3 (PBX3), | | |
| | | | | | mRNA. | | |
| 706 | 686 | 2480039 | NM_015878.4 | NM_015878 | antizyme inhibitor 1 | AZIN1 | ODC1L; OAZIN; |
| | | | | | (AZIN1), transcript | | OAZI; MGC3832; |
| | | | | | variant 1, mRNA. | | MGC691 |
| 707 | 687 | 2480048 | NM_178124.3 | NM_178124 | chromosome X open | CXorf40A | EOLA1; CXorf40 |
| | | | | | reading frame 40A | | |
| | | | | | (CXorf40A), mRNA. | | |
| 708 | 688 | 2480075 | NM_024997.2 | NM_024997 | activating transcription | ATF7IP2 | MCAF2; FLJ12668 |
| | | | | | factor 7 interacting | | |
| | | | | | protein 2 (ATF7IP2), | | |
| | | | | | mRNA. | | |
| 709 | 689 | 2480424 | NM_002661.2 | NM_002661 | phospholipase C, | PLCG2 | — |
| | | | | | gamma 2 | | |
| | | | | | (phosphatidylinositol- | | |
| | | | | | specific) (PLCG2), | | |
| | | | | | mRNA. | | |
| 710 | 690 | 2480576 | NM_001033566.1 | NM_001033566 | ras homolog gene | RHOT1 | ARHT1; MIRO-1; |
| | | | | | family, member T1 | | FLJ12633; FLJ11040 |
| | | | | | (RHOT1), transcript | | |
| | | | | | variant 2, mRNA. | | |
| 711 | 691 | 2490072 | NM_032152.3 | NM_032152 | PML-RARA regulated | PRAM1 | MGC39864; PRAM-1 |
| | | | | | adaptor molecule 1 | | |
| | | | | | (PRAM1), mRNA. | | |
| 712 | 692 | 2490240 | NM_130435.2 | NM_130435 | protein tyrosine | PTPRE | DKFZp313F1310; |
| | | | | | phosphatase, receptor | | PTPE; HPTPE; R- |
| | | | | | type, E (PTPRE), | | PTP-EPSILON |
| | | | | | transcript variant 2, | | |
| | | | | | mRNA. | | |
| 713 | 693 | 2490452 | NM_000152.3 | NM_000152 | glucosidase, alpha; | GAA | LYAG |
| | | | | | acid (Pompe disease, | | |
| | | | | | glycogen storage | | |
| | | | | | disease type II) (GAA), | | |
| | | | | | transcript variant 1, | | |
| | | | | | mRNA. | | |
| 714 | 694 | 2490598 | NM_001155.3 | NM_001155 | annexin A6 (ANXA6), | ANXA6 | ANX6; CBP68 |
| | | | | | transcript variant 1, | | |
| | | | | | mRNA. | | |
| 715 | 695 | 2490719 | NM_004226.2 | NM_004226 | serine/threonine kinase | STK17B | DRAK2 |
| | | | | | 17b (STK17B), mRNA. | | |
| 716 | 696 | 2510086 | NM_138473.2 | NM_138473 | Sp1 transcription factor | SP1 | — |
| | | | | | (SP1), mRNA. | | |
| 717 | 697 | 2510278 | NM_006341.2 | NM_006341 | MAD2 mitotic arrest | MAD2L2 | REV7; MAD2B |
| | | | | | deficient-like 2 (yeast) | | |
| | | | | | (MAD2L2), mRNA. | | |
| 718 | 698 | 2570112 | NM_006720.3 | NM_006720 | actin binding LIM | ABLIM1 | KIAA0059; FLJ14564; |
| | | | | | protein 1 (ABLIM1), | | MGC1224; LIMATIN; |
| | | | | | transcript variant 4, | | LIMAB1; |
| | | | | | mRNA. | | DKFZp781D0148; |
| | | | | | | | ABLIM |
| 719 | 699 | 2570253 | NM_007047.3 | NM_007047 | butyrophilin, subfamily | BTN3A2 | BT3.3; BT3.2; BTF4 |
| | | | | | 3, member A2 | | |
| | | | | | (BTN3A2), mRNA. | | |
| 720 | 700 | 2570433 | NM_145341.2 | NM_145341 | programmed cell death | PDCD4 | MGC33046; |
| | | | | | 4 (neoplastic | | MGC33047; H731 |
| | | | | | transformation inhibitor) | | |
| | | | | | (PDCD4), transcript | | |
| | | | | | variant 2, mRNA. | | |
| 721 | 701 | 2600138 | NM_006123.2 | NM_006123 | iduronate 2-sulfatase | IDS | MPS2; SIDS |
| | | | | | (Hunter syndrome) | | |
| | | | | | (IDS), transcript variant | | |
| | | | | | 2, mRNA. | | |
| 722 | 702 | 2600537 | NM_001755.2 | NM_001755 | core-binding factor, | CBFB | PEBP2B |
| | | | | | beta subunit (CBFB), | | |
| | | | | | transcript variant 2, | | |
| | | | | | mRNA. | | |
| 723 | 703 | 2600747 | NM_001547.4 | NM_001547 | interferon-induced | IFIT2 | GARG-39; G10P2; |
| | | | | | protein with | | cig42; ISG-54K; IFI- |
| | | | | | tetratricopeptide | | 54; IFI54; ISG54 |
| | | | | | repeats 2 (IFIT2), | | |
| | | | | | mRNA. | | |
| 724 | 704 | 2630008 | NM_144576.3 | NM_144576 | coenzyme Q10 | COQ10A | FLJ32452 |
| | | | | | homolog A (S. cerevisiae) | | |
| | | | | | (COQ10A), | | |
| | | | | | transcript variant 1, | | |
| | | | | | mRNA. | | |
| 725 | 705 | 2630370 | NM_001013836.1 | NM_001013836 | MAD1 mitotic arrest | MAD1L1 | PIG9; HsMAD1; |
| | | | | | deficient-like 1 (yeast) | | TXBP181; MAD1; |
| | | | | | (MAD1L1), transcript | | TP53I9 |
| | | | | | variant 2, mRNA. | | |
| 726 | 706 | 2630554 | NM_152405.2 | NM_152405 | junction-mediating and | JMY | FLJ37870; |
| | | | | | regulatory protein | | MGC163496 |
| | | | | | (JMY), mRNA. | | |
| 727 | 708 | 2640068 | NM_002934.2 | NM_002934 | ribonuclease, RNase A | RNASE2 | EDN; RNS2 |
| | | | | | family, 2 (liver, | | |
| | | | | | eosinophil-derived | | |
| | | | | | neurotoxin) (RNASE2), | | |
| | | | | | mRNA. | | |
| 728 | 709 | 2640161 | NM_206962.1 | NM_206962 | protein arginine | PRMT2 | HRMT1L1; |
| | | | | | methyltransferase 2 | | MGC111373 |
| | | | | | (PRMT2), transcript | | |
| | | | | | variant 1, mRNA. | | |
| 729 | 710 | 2640286 | NM_000517.3 | NM_000517 | hemoglobin, alpha 2 | HBA2 | HBA1 |
| | | | | | (HBA2), mRNA. | | |
| 730 | 711 | 2640471 | NM_007219.2 | NM_007219 | ring finger protein 24 | RNF24 | G1L |
| | | | | | (RNF24), mRNA. | | GCACCAGTAAGGCCCGT |
| 731 | 712 | 2640551 | NM_016308.1 | NM_016308 | cytidine | CMPK1 | RP11-511I2.1; UMK; |
| | | | | | monophosphate (UMP- | | UMP-CMPK; CMPK; |
| | | | | | CMP) kinase 1, | | CMK; UMPK |
| | | | | | cytosolic (CMPK1), | | |
| | | | | | mRNA. | | |
| 732 | 713 | 2640619 | NM_003430.2 | NM_003430 | zinc finger protein 91 | ZNF91 | HTF10; HPF7 |
| | | | | | (ZNF91), mRNA. | | |
| 733 | 714 | 2650113 | NM_033013.1 | NM_033013 | nuclear receptor | NR1I2 | PAR; PRR; SAR; |
| | | | | | subfamily 1, group I, | | PAR1; ONR1; BXR; |
| | | | | | member 2 (NR1I2), | | SXR; PXR; PAR2; |
| | | | | | transcript variant 3, | | PARq |
| | | | | | mRNA. | | |
| 734 | 715 | 2650468 | NM_001037637.1 | NM_001037637 | basic transcription | BTF3 | BETA-NAC; BTF3a; |
| | | | | | factor 3 (BTF3), | | BTF3b; NACB |
| | | | | | transcript variant 1, | | |
| | | | | | mRNA. | | |
| 735 | 716 | 2650564 | NM_004585.3 | NM_004585 | retinoic acid receptor | RARRES3 | MGC8906; |
| | | | | | responder (tazarotene | | HRASLS4; TIG3; |
| | | | | | induced) 3 | | RIG1 |
| | | | | | (RARRES3), mRNA. | | |
| 736 | 717 | 2650594 | NM_001080156.1 | NM_001080156 | Rho GTPase activating | ARHGAP9 | 10C; RGL1; |
| | | | | | protein 9 (ARHGAP9), | | FLJ16525; MGC1295 |
| | | | | | transcript variant 3, | | |
| | | | | | mRNA. | | |
| 737 | 718 | 2650619 | NM_006597.3 | NM_006597 | heat shock 70 kDa | HSPA8 | MGC131511; HSC54; |
| | | | | | protein 8 (HSPA8), | | HSPA10; MGC29929; |
| | | | | | transcript variant 1, | | HSC70; HSP71; |
| | | | | | mRNA. | | HSP73; LAP1; |
| | | | | | | | HSC71; NIP71 |
| 738 | 719 | 2680050 | NM_080816.2 | NM_080816 | signal-regulatory | SIRPG | SIRPgamma; SIRP- |
| | | | | | protein gamma | | B2; CD172g; SIRPB2; |
| | | | | | (SIRPG), transcript | | bA77C3.1 |
| | | | | | variant 2, mRNA. | | |
| 739 | 720 | 2680056 | NM_001025195.1 | NM_001025195 | carboxylesterase 1 | CES1 | HMSE1; PCE-1; |
| | | | | | (monocyte/macrophage | | CES2; TGH; SES1; |
| | | | | | serine esterase 1) | | HMSE; ACAT; |
| | | | | | (CES1), transcript | | MGC117365; CEH |
| | | | | | variant 1, mRNA. | | |
| 740 | 721 | 2680092 | NM_203416.1 | NM_203416 | CD163 molecule | CD163 | MM130; M130 |
| | | | | | (CD163), transcript | | |
| | | | | | variant 2, mRNA. | | |
| 741 | 722 | 2680446 | NM_001659.1 | NM_001659 | ADP-ribosylation factor | ARF3 | — |
| | | | | | 3 (ARF3), mRNA. | | |
| 742 | 723 | 2680639 | NM_017875.1 | NM_017875 | solute carrier family 25, | SLC25A38 | FLJ20551; FLJ22703 |
| | | | | | member 38 | | |
| | | | | | (SLC25A38), mRNA. | | |
| 743 | 724 | 2690068 | NM_145863.1 | NM_145863 | ankyrin repeat and | ASB3 | FLJ10123; ASB-3; |
| | | | | | SOCS box-containing 3 | | MGC996; |
| | | | | | (ASB3), transcript | | MGC132002; |
| | | | | | variant 2, mRNA. | | FLJ10421; |
| | | | | | | | MGC12531 |
| 744 | 725 | 2690520 | NM_021930.4 | NM_021930 | RAD50 interactor 1 | RINT1 | DKFZp667H2324; |
| | | | | | (RINT1), mRNA. | | RINT-1 |
| 745 | 726 | 2690524 | NM_006698.2 | NM_006698 | bladder cancer | BLCAP | BC10 |
| | | | | | associated protein | | |
| | | | | | (BLCAP), mRNA. | | |
| 746 | 727 | 2690598 | NM_021626.1 | NM_021626 | serine | SCPEP1 | RISC; HSCP1 |
| | | | | | carboxypeptidase 1 | | |
| | | | | | (SCPEP1), mRNA. | | |
| 747 | 728 | 2710068 | NM_003757.2 | NM_003757 | eukaryotic translation | EIF3I | EIF3S2; TRIP-1; eIF3- |
| | | | | | initiation factor 3, | | beta; TRIP1; eIF3i; |
| | | | | | subunit I (EIF3I), | | eIF3-p36; PRO2242 |
| | | | | | mRNA. | | |
| 748 | 729 | 2710129 | NM_152783.3 | NM_152783 | D-2-hydroxyglutarate | D2HGDH | FLJ42195; |
| | | | | | dehydrogenase | | MGC25181; D2HGD |
| | | | | | (D2HGDH), nuclear | | |
| | | | | | gene encoding | | |
| | | | | | mitochondrial protein, | | |
| | | | | | mRNA. | | |
| 749 | 730 | 2710286 | NM_002332.2 | NM_002332 | low density lipoprotein- | LRP1 | A2MR; FLJ16451; |
| | | | | | related protein 1 (alpha- | | LRP; TGFBR5; CD91; |
| | | | | | 2-macroglobulin | | APR; MGC88725; |
| | | | | | receptor) (LRP1), | | APOER |
| | | | | | mRNA. | | |
| 750 | 731 | 2710400 | NM_004949.2 | NM_004949 | desmocollin 2 (DSC2), | DSC2 | DGII/III; |
| | | | | | transcript variant | | DKFZp686I11137; |
| | | | | | Dsc2b, mRNA. | | CDHF2; ARVD11; |
| | | | | | | | DG2; DSC3 |
| 751 | 732 | 2710653 | NM_030793.3 | NM_030793 | F-box protein 38 | FBXO38 | Fbx38; SP329; MOKA |
| | | | | | (FBXO38), transcript | | |
| | | | | | variant 1, mRNA. | | |
| 752 | 733 | 2710682 | NM_000904.2 | NM_000904 | NAD(P)H | NQO2 | DHQV; DIA6; |
| | | | | | dehydrogenase, | | NMOR2; QR2 |
| | | | | | quinone 2 (NQO2), | | |
| | | | | | mRNA. | | |
| 753 | 734 | 2710754 | NM_198196.2 | NM_198196 | CD96 molecule | CD96 | DKFZp667E2122; |
| | | | | | (CD96), transcript | | MGC22596; TACTILE |
| | | | | | variant 1, mRNA. | | |
| 754 | 735 | 2750154 | NM_014800.9 | NM_014800 | engulfment and cell | ELMO1 | CED-12; KIAA0281; |
| | | | | | motility 1 (ELMO1), | | ELMO-1; |
| | | | | | transcript variant 1, | | MGC126406; CED12 |
| | | | | | mRNA. | | |
| 755 | 737 | 2750750 | NM_003343.4 | NM_003343 | ubiquitin-conjugating | UBE2G2 | UBC7 |
| | | | | | enzyme E2G 2 (UBC7 | | |
| | | | | | homolog, yeast) | | |
| | | | | | (UBE2G2), transcript | | |
| | | | | | variant 1, mRNA. | | |
| 756 | 738 | 2760164 | NM_012328.1 | NM_012328 | DnaJ (Hsp40) homolog, | DNAJB9 | MSTP049; MDG1; |
| | | | | | subfamily B, member 9 | | DKFZP564F1862; |
| | | | | | (DNAJB9), mRNA. | | MST049; ERdj4 |
| 757 | 739 | 2760201 | XM_001133926.1 | XM_001133926 | PREDICTED: solute | SLC25A20 | — |
| | | | | | carrier family 25 | | |
| | | | | | (carnitine/acylcarnitinetranslocase), | | |
| | | | | | member 20 | | |
| | | | | | (SLC25A20), mRNA. | | |
| 758 | 740 | 2760255 | NM_007358.2 | NM_007358 | metal response | MTF2 | dJ976O13.2; PCL2; |
| | | | | | element binding | | M96; RP5-976013.1 |
| | | | | | transcription factor 2 | | |
| | | | | | (MTF2), mRNA. | | |
| 759 | 741 | 2760427 | NM_002863.3 | NM_002863 | phosphorylase, | PYGL | — |
| | | | | | glycogen; liver (Hers | | |
| | | | | | disease, glycogen | | |
| | | | | | storage disease type | | |
| | | | | | VI) (PYGL), mRNA. | | |
| 760 | 742 | 2810202 | NM_001009936.1 | NM_001009936 | PHD finger protein 19 | PHF19 | MGC131698; PCL3; |
| | | | | | (PHF19), transcript | | MGC23929; |
| | | | | | variant 2, mRNA. | | MGC149713; |
| | | | | | | | MGC149712 |
| 761 | 743 | 2810246 | NM_030915.1 | NM_030915 | limb bud and heart | LBH | MGC163287; |
| | | | | | development homolog | | MGC104312; |
| | | | | | (mouse) (LBH), mRNA. | | DKFZP566J091 |
| 762 | 744 | 2810296 | NM_000709.2 | NM_000709 | branched chain keto | BCKDHA | FLJ45695; MSUD1; |
| | | | | | acid dehydrogenase | | MSU |
| | | | | | E1, alpha polypeptide | | |
| | | | | | (BCKDHA), mRNA. | | |
| 763 | 745 | 2810315 | NM_014624.3 | NM_014624 | S100 calcium binding | S100A6 | PRA; CABP; 2A9; |
| | | | | | protein A6 (S100A6), | | 5B10; CACY |
| | | | | | mRNA. | | |
| 764 | 746 | 2810328 | NM_006047.4 | NM_006047 | RNA binding motif | RBM12 | KIAA0765; |
| | | | | | protein 12 (RBM12), | | HRIHFB2091; SWAN |
| | | | | | transcript variant 1, | | |
| | | | | | mRNA. | | |
| 765 | 747 | 2810364 | NM_014315.2 | NM_014315 | kelch domain | KLHDC2 | LCP; HCLP-1 |
| | | | | | containing 2 (KLHDC2), | | |
| | | | | | mRNA. | | |
| 766 | 748 | 2810601 | NM_016269.2 | NM_016269 | lymphoid enhancer- | LEF1 | TCF1ALPHA; |
| | | | | | binding factor 1 (LEF1), | | DKFZp586H0919 |
| | | | | | mRNA. | | |
| 767 | 749 | 2810661 | NM_001080435.1 | NM_001080435 | WAS protein homology | WHDC1 | KIAA1971 |
| | | | | | region 2 domain | | |
| | | | | | containing 1 (WHDC1), | | |
| | | | | | mRNA. | | |
| 768 | 750 | 2810678 | NM_176800.1 | NM_176800 | PRP4 pre-mRNA | PRPF4B | PR4H; dJ1013A10.1; |
| | | | | | processing factor 4 | | PRP4; KIAA0536; |
| | | | | | homolog B (yeast) | | PRP4H; PRP4K |
| | | | | | (PRPF4B), transcript | | |
| | | | | | variant 2, mRNA. | | |
| 769 | 751 | 2850575 | NM_001033.2 | NM_001033 | ribonucleotide | RRM1 | RR1; R1; RIR1 |
| | | | | | reductase M1 | | |
| | | | | | polypeptide (RRM1), | | |
| | | | | | mRNA. | | |
| 770 | 752 | 2850576 | NM_022367.2 | NM_022367 | sema domain, | SEMA4A | RP11-54H19.2; |
| | | | | | immunoglobulin domain | | RP35; SEMB; |
| | | | | | (Ig), transmembrane | | SEMAB; FLJ12287; |
| | | | | | domain (TM) and short | | CORD10 |
| | | | | | cytoplasmic domain, | | |
| | | | | | (semaphorin) 4A | | |
| | | | | | (SEMA4A), mRNA. | | |
| 771 | 753 | 2900768 | NM_021737.1 | NM_021737 | chloride channel 6 | CLCN6 | KIAA0046; CLC-6 |
| | | | | | (CLCN6), transcript | | |
| | | | | | variant CIC-6d, mRNA. | | |
| 772 | 755 | 2970026 | NM_033414.2 | NM_033414 | zinc finger protein 622 | ZNF622 | MGC17552; ZPR9; |
| | | | | | (ZNF622), mRNA. | | MGC2485 |
| 773 | 756 | 2970133 | NM_005049.2 | NM_005049 | PWP2 periodic | PWP2 | PWP2H; EHOC-17 |
| | | | | | tryptophan protein | | |
| | | | | | homolog (yeast) | | |
| | | | | | (PWP2), mRNA. | | |
| 774 | 758 | 3060092 | NM_022040.2 | NM_022040 | linker for activation of T | LAT2 | WBSCR5; |
| | | | | | cells family, member 2 | | WBSCR15; |
| | | | | | (LAT2), transcript | | HSPC046; NTAL; |
| | | | | | variant 1, mRNA. | | WSCR5; LAB |
| 775 | 759 | 3060278 | NM_138381.1 | NM_138381 | hypothetical protein | MGC15763 | — |
| | | | | | BC008322 | | |
| | | | | | (MGC15763), mRNA. | | |
| 776 | 760 | 3060487 | NM_002802.2 | NM_002802 | proteasome (prosome, | PSMC1 | P26S4; p56; |
| | | | | | macropain) 26S | | MGC24583; S4; |
| | | | | | subunit, ATPase, 1 | | MGC8541 |
| | | | | | (PSMC1), mRNA. | | |
| 777 | 761 | 3060494 | NM_052813.3 | NM_052813 | caspase recruitment | CARD9 | hCARD9 |
| | | | | | domain family, member | | |
| | | | | | 9 (CARD9), mRNA. | | |
| 778 | 762 | 3060612 | NM_002115.1 | NM_002115 | hexokinase 3 (white | HK3 | HXK3; HKIII |
| | | | | | cell) (HK3), nuclear | | |
| | | | | | gene encoding | | |
| | | | | | mitochondrial protein, | | |
| | | | | | mRNA. | | |
| 779 | 763 | 3060692 | NM_182661.1 | NM_182661 | ceramide kinase | CERK | KIAA1646; |
| | | | | | (CERK), transcript | | dA59H18.3; hCERK; |
| | | | | | variant 2, mRNA. | | dA59H18.2; |
| | | | | | | | FLJ23239; |
| | | | | | | | MGC131878; LK4; |
| | | | | | | | FLJ21430; |
| | | | | | | | DKFZp434E0211 |
| 780 | 764 | 3120053 | NM_015888.4 | NM_015888 | hook homolog 1 | HOOK1 | MGC10642; HK1 |
| | | | | | (Drosophila) (HOOK1), | | |
| | | | | | mRNA. | | |
| 781 | 765 | 3120228 | NM_004069.3 | NM_004069 | adaptor-related protein | AP2S1 | AP17; CLAPS2; |
| | | | | | complex 2, sigma 1 | | AP17-DELTA |
| | | | | | subunit (AP2S1), | | |
| | | | | | transcript variant AP17, | | |
| | | | | | mRNA. | | |
| 782 | 766 | 3120370 | NM_001251.1 | NM_001251 | CD68 antigen (CD68), | CD68 | SCARD1 |
| | | | | | mRNA. | | |
| 783 | 767 | 3120431 | NM_000883.2 | NM_000883 | IMP (inosine | IMPDH1 | sWSS2608; RP10; |
| | | | | | monophosphate) | | DKFZp781N0678; |
| | | | | | dehydrogenase 1 | | LCA11; IMPD; IMPD1 |
| | | | | | (IMPDH1), transcript | | |
| | | | | | variant 1, mRNA. | | |
| 784 | 768 | 3120707 | NM_001014840.1 | NM_001014840 | cutA divalent cation | CUTA | MGC111154; |
| | | | | | tolerance homolog (E. coli) | | ACHAP; C6orf82 |
| | | | | | (CUTA), transcript | | |
| | | | | | variant 5, mRNA. | | |
| 785 | 769 | 3130079 | NM_014239.2 | NM_014239 | eukaryotic translation | EIF2B2 | EIF-2Bbeta; EIF2B |
| | | | | | initiation factor 2B, | | |
| | | | | | subunit 2 beta, 39 kDa | | |
| | | | | | (EIF2B2), mRNA. | | |
| 786 | 770 | 3130136 | NM_002649.2 | NM_002649 | phosphoinositide-3- | PIK3CG | PIK3; PI3K; PI3CG; |
| | | | | | kinase, catalytic, | | PI3Kgamma |
| | | | | | gamma polypeptide | | |
| | | | | | (PIK3CG), mRNA. | | |
| 787 | 771 | 3130246 | NM_173647.2 | NM_173647 | ring finger protein 149 | RNF149 | FLJ90504; DNAPTP2 |
| | | | | | (RNF149), mRNA. | | |
| 788 | 772 | 3130301 | NM_002648.2 | NM_002648 | pim-1 oncogene | PIM1 | PIM |
| | | | | | (PIM1), mRNA. | | |
| 789 | 773 | 3130768 | NM_004487.3 | NM_004487 | golgin B1, golgi integral | GOLGB1 | GOLIM1; GCP372; |
| | | | | | membrane protein | | GCP; GIANTIN |
| | | | | | (GOLGB1), mRNA. | | |
| 790 | 775 | 3140553 | NM_020150.3 | NM_020150 | SAR1 gene homolog A | SAR1A | masra2; SARA1; |
| | | | | | (S. cerevisiae) | | SAR1; Sara |
| | | | | | (SAR1A), mRNA. | | |
| 791 | 776 | 3140670 | NM_000117.1 | NM_000117 | emerin (Emery-Dreifuss | EMD | STA; EDMD |
| | | | | | muscular dystrophy) | | |
| | | | | | (EMD), mRNA. | | |
| 792 | 777 | 3140707 | NM_031458.1 | NM_031458 | poly (ADP-ribose) | PARP9 | DKFZp666B0810; |
| | | | | | polymerase family, | | DKFZp686M15238; |
| | | | | | member 9 (PARP9), | | BAL1; FLJ26637; |
| | | | | | mRNA. | | BAL; FLJ41418; |
| | | | | | | | MGC: 7868 |
| 793 | 778 | 3170519 | NM_012208.2 | NM_012208 | histidyl-tRNAsynthetase | HARS2 | HO3; HARSL; |
| | | | | | 2, mitochondrial | | HARSR |
| | | | | | (putative) (HARS2), | | |
| | | | | | nuclear gene encoding | | |
| | | | | | mitochondrial protein, | | |
| | | | | | mRNA. | | |
| 794 | 779 | 3180215 | NM_033657.1 | NM_033657 | death associated | DAP3 | MGC126059; |
| | | | | | protein 3 (DAP3), | | MGC126058; bMRP- |
| | | | | | nuclear gene encoding | | 10; MRP-S29; |
| | | | | | mitochondrial protein, | | DKFZp686G12159; |
| | | | | | transcript variant 1, | | DAP-3; MRPS29 |
| | | | | | mRNA. | | |
| 795 | 780 | 3180289 | NM_001987.4 | NM_001987 | ets variant gene 6 (TEL | ETV6 | TEL; TEL/ABL |
| | | | | | oncogene) (ETV6), | | |
| | | | | | mRNA. | | |
| 796 | 781 | 3180438 | NM_001006.3 | NM_001006 | ribosomal protein S3A | RPS3A | FTE1; MGC23240; |
| | | | | | (RPS3A), mRNA. | | MFTL |
| 797 | 782 | 3180446 | NM_005481.2 | NM_005481 | mediator complex | MED16 | THRAP5; TRAP95; |
| | | | | | subunit 16 (MED16), | | DRIP92; MED16 |
| | | | | | mRNA. | | |
| 798 | 783 | 3180681 | NR_003187.1 | NR_003187 | neutrophil cytosolic | NCF1C | SH3PXD1C |
| | | | | | factor 1C pseudogene | | |
| | | | | | (NCF1C) on | | |
| | | | | | chromosome 7. | | |
| 799 | 784 | 3180736 | NM_199002.1 | NM_199002 | Rho guanine nucleotide | ARHGEF1 | P115-RHOGEF; |
| | | | | | exchange factor (GEF) | | GEF1; LBCL2; |
| | | | | | 1 (ARHGEF1), | | SUB1.5 |
| | | | | | transcript variant 1, | | |
| | | | | | mRNA. | | |
| 800 | 785 | 3190437 | NM_138361.3 | NM_138361 | leucine rich repeat and | LRSAM1 | FLJ31641; RIFLE; |
| | | | | | sterile alpha motif | | TAL |
| | | | | | containing 1 | | |
| | | | | | (LRSAM1), transcript | | |
| | | | | | variant 1, mRNA. | | |
| 801 | 786 | 3290019 | NM_024814.1 | NM_024814 | Cas-Br-M (murine) | CBLL1 | HAKAI; FLJ23109; |
| | | | | | ecotropic retroviral | | MGC163403; |
| | | | | | transforming sequence- | | RNF188; |
| | | | | | like 1 (CBLL1), mRNA. | | MGC163401 |
| 802 | 787 | 3290292 | NM_015907.2 | NM_015907 | leucineaminopeptidase | LAP3 | LAP; LAPEP; PEPS |
| | | | | | 3 (LAP3), mRNA. | | |
| 803 | 788 | 3290315 | NM_002525.1 | NM_002525 | nardilysin (N-arginine | NRD1 | hNRD2; hNRD1 |
| | | | | | dibasic convertase) | | |
| | | | | | (NRD1), mRNA. | | |
| 804 | 789 | 3290669 | NM_139168.2 | NM_139168 | splicing factor, | SFRS12 | DKFZp564B176; |
| | | | | | arginine/serine-rich 12 | | SRrp508; SRrp86; |
| | | | | | (SFRS12), transcript | | MGC133045 |
| | | | | | variant 2, mRNA. | | |
| 805 | 790 | 3310091 | NM_018643.2 | NM_018643 | triggering receptor | TREM1 | TREM-1 |
| | | | | | expressed on myeloid | | |
| | | | | | cells 1 (TREM1), | | |
| | | | | | mRNA. | | |
| 806 | 791 | 3310093 | NM_017861.1 | NM_017861 | phosphatidylinositol | PIGX | FLJ20522 |
| | | | | | glycan anchor | | |
| | | | | | biosynthesis, class X | | |
| | | | | | (PIGX), mRNA. | | |
| 807 | 792 | 3310358 | NM_000294.1 | NM_000294 | phosphorylase kinase, | PHKG2 | — |
| | | | | | gamma 2 (testis) | | |
| | | | | | (PHKG2), mRNA. | | |
| 808 | 793 | 3360634 | NM_013322.2 | NM_013322 | sorting nexin 10 | SNX10 | MGC33054 |
| | | | | | (SNX10), mRNA. | | |
| 809 | 794 | 3370093 | NM_013304.1 | NM_013304 | zinc finger, DHHC-type | ZDHHC1 | ZNF377; C16orf1; |
| | | | | | containing 1 | | HSU90653 |
| | | | | | (ZDHHC1), mRNA. | | |
| 810 | 795 | 3370112 | NM_002951.2 | NM_002951 | ribophorin II (RPN2), | RPN2 | RPNII; RIBIIR; RPN- |
| | | | | | mRNA. | | II; SWP1 |
| 811 | 796 | 3370202 | NR_001562.1 | NR_001562 | annexin A2 | ANXA2P1 | LPC2A; ANX2L1; |
| | | | | | pseudogene 1 | | ANX2P1 |
| | | | | | (ANXA2P1) on | | |
| | | | | | chromosome 4. | | |
| 812 | 797 | 3370300 | NM_133459.1 | NM_133459 | collagen and calcium | CCBE1 | FLJ30681; |
| | | | | | binding EGF domains 1 | | MGC50861 |
| | | | | | (CCBE1), mRNA. | | |
| 813 | 798 | 3370327 | NM_022746.2 | NM_022746 | MOCO sulphurase C- | MOSC1 | FLJ22390; RP11- |
| | | | | | terminal domain | | 295M18.1 |
| | | | | | containing 1 (MOSC1), | | |
| | | | | | mRNA. | | |
| 814 | 799 | 3370594 | NM_006866.1 | NM_006866 | leukocyte | LILRA2 | CD85H; LIR-7; LIR7; |
| | | | | | immunoglobulin-like | | ILT1 |
| | | | | | receptor, subfamily A | | |
| | | | | | (with TM domain), | | |
| | | | | | member 2 (LILRA2), | | |
| | | | | | mRNA. | | |
| 815 | 800 | 3390022 | NM_001861.2 | NM_001861 | cytochrome c oxidase | COX4I1 | MGC72016; COXIV; |
| | | | | | subunit IV isoform 1 | | COX4 |
| | | | | | (COX4I1), mRNA. | | |
| 816 | 801 | 3390093 | NM_001035505.1 | NM_001035505 | bolA homolog 3 (E. coli) | BOLA3 | — |
| | | | | | (BOLA3), transcript | | |
| | | | | | variant 2, mRNA. | | |
| 817 | 802 | 3390286 | NM_000682.5 | NM_000682 | adrenergic, alpha-2B-, | ADRA2B | ADRARL1; |
| | | | | | receptor (ADRA2B), | | ADRA2L1; |
| | | | | | mRNA. | | ALPHA2BAR; |
| | | | | | | | ADRA2RL1 |
| 818 | 803 | 3390544 | NM_001017963.2 | NM_001017963 | heat shock protein | HSP90AA1 | HSPCAL4; HSPN; |
| | | | | | 90 kDa alpha | | HSP86; Hsp89; |
| | | | | | (cytosolic), class A | | HSP90N; HSPC1; |
| | | | | | member 1 | | HSP90A; HSPCAL1; |
| | | | | | (HSP90AA1), transcript | | Hsp90; HSPCA; |
| | | | | | variant 1, mRNA. | | LAP2; FLJ31884 |
| 819 | 804 | 3390603 | NM_005917.2 | NM_005917 | malate dehydrogenase | MDH1 | MOR2; MDH-s; |
| | | | | | 1, NAD (soluble) | | MGC: 1375; MDHA |
| | | | | | (MDH1), mRNA. | | |
| 820 | 805 | 3390722 | NM_032316.3 | NM_032316 | nicolin 1 (NICN1), | NICN1 | MGC12936 |
| | | | | | mRNA. | | |
| 821 | 806 | 3400097 | NM_001098516.1 | NM_001098516 | mucin 20, cell surface | MUC20 | FLJ14408; KIAA1359 |
| | | | | | associated (MUC20), | | |
| | | | | | transcript variant S, | | |
| | | | | | mRNA. | | |
| 822 | 807 | 3400408 | NM_052935.2 | NM_052935 | 5′-nucleotidase, | NT5C3L | MGC20781; |
| | | | | | cytosolic III-like | | MGC21375 |
| | | | | | (NT5C3L), mRNA. | | |
| 823 | 808 | 3400612 | NM_020830.3 | NM_020830 | WD repeat and FYVE | WDFY1 | FENS-1; ZFYVE17; |
| | | | | | domain containing 1 | | WDF1 |
| | | | | | (WDFY1), mRNA. | | |
| 824 | 809 | 3420020 | NM_003082.2 | NM_003082 | small nuclear RNA | SNAPC1 | SNAP43; PTFgamma |
| | | | | | activating complex, | | |
| | | | | | polypeptide 1, 43 kDa | | |
| | | | | | (SNAPC1), mRNA. | | |
| 825 | 810 | 3420253 | NM_001033505.1 | NM_001033505 | cleavage stimulation | CSTF3 | MGC75122; |
| | | | | | factor, 3′ pre-RNA, | | MGC43001; |
| | | | | | subunit 3, 77 kDa | | MGC117398; CSTF- |
| | | | | | (CSTF3), transcript | | 77 |
| | | | | | variant 2, mRNA. | | |
| 826 | 811 | 3420523 | NM_001005498.2 | NM_001005498 | rhomboid 5 homolog 2 | RHBDF2 | RHBDL6; RHBDL5; |
| | | | | | (Drosophila) | | FLJ22341 |
| | | | | | (RHBDF2), transcript | | |
| | | | | | variant 2, mRNA. | | |
| 827 | 812 | 3420632 | NM_148919.3 | NM_148919 | proteasome (prosome, | PSMB8 | D6S216; LMP7; |
| | | | | | macropain) subunit, | | PSMB5i; RING10; |
| | | | | | beta type, 8 (large | | beta5i; MGC1491; |
| | | | | | multifunctional | | D6S216E |
| | | | | | peptidase 7) (PSMB8), | | |
| | | | | | transcript variant 2, | | |
| | | | | | mRNA. | | |
| 828 | 813 | 3420747 | NM_001121.2 | NM_001121 | adducin 3 (gamma) | ADD3 | ADDL |
| | | | | | (ADD3), transcript | | |
| | | | | | variant 3, mRNA. | | |
| 829 | 814 | 3440070 | NM_004890.2 | NM_004890 | sperm associated | SPAG7 | ACRP; FSA-1; |
| | | | | | antigen 7 (SPAG7), | | MGC20134 |
| | | | | | mRNA. | | |
| 830 | 815 | 3440204 | NM_001999.3 | NM_001999 | fibrillin 2 (congenital | FBN2 | CCA |
| | | | | | contracturalarachnodactyly) | | |
| | | | | | (FBN2), mRNA. | | |
| 831 | 816 | 3440452 | NM_207195.1 | NM_207195 | ADAM | ADAM15 | MDC15 |
| | | | | | metallopeptidase | | |
| | | | | | domain 15 (ADAM15), | | |
| | | | | | transcript variant 4, | | |
| | | | | | mRNA. | | |
| 832 | 817 | 3440491 | NM_000153.2 | NM_000153 | galactosylceramidase | GALC | — |
| | | | | | (GALC), transcript | | |
| | | | | | variant 1, mRNA. | | |
| 833 | 818 | 3440739 | NM_000518.4 | NM_000518 | hemoglobin, beta | HBB | HBD; CD113t-C |
| | | | | | (HBB), mRNA. | | |
| 834 | 819 | 3440754 | NM_001012633.1 | NM_001012633 | interleukin 32 (IL32), | IL32 | IL-32alpha; TAIFd; IL- |
| | | | | | transcript variant 4, | | 32beta; TAIFc; TAIF; |
| | | | | | mRNA. | | IL-32gamma; TAIFb; |
| | | | | | | | TAIFa; IL-32delta; |
| | | | | | | | NK4 |
| 835 | 820 | 3450138 | NM_001814.2 | NM_001814 | cathepsin C (CTSC), | CTSC | JPD; DPP1; JP; HMS; |
| | | | | | transcript variant 1, | | CPPI; PALS; PLS; |
| | | | | | mRNA. | | DPPI |
| 836 | 821 | 3450253 | NM_004773.2 | NM_004773 | zinc finger, HIT type 3 | ZNHIT3 | TRIP3 |
| | | | | | (ZNHIT3), mRNA. | | |
| 837 | 822 | 3450280 | NM_016274.4 | NM_016274 | pleckstrin homology | PLEKHO1 | CKIP-1; OC120; |
| | | | | | domain containing, | | RP11-458I7.3 |
| | | | | | family O member 1 | | |
| | | | | | (PLEKHO1), mRNA. | | |
| 838 | 823 | 3450427 | NM_153450.1 | NM_153450 | mediator complex | MED19 | LCMR1 |
| | | | | | subunit 19 (MED19), | | |
| | | | | | mRNA. | | |
| 839 | 824 | 3450521 | NM_022664.1 | NM_022664 | extracellular matrix | ECM1 | — |
| | | | | | protein 1 (ECM1), | | |
| | | | | | transcript variant 2, | | |
| | | | | | mRNA. | | |
| 840 | 825 | 3450719 | NM_001402.5 | NM_001402 | eukaryotic translation | EEF1A1 | EEF1A; FLJ25721; |
| | | | | | elongation factor 1 | | CCS-3; PTI1; CCS3; |
| | | | | | alpha 1 (EEF1A1), | | MGC102687; |
| | | | | | mRNA. | | MGC16224; EF-Tu; |
| | | | | | | | eEF1A-1; EEF-1; |
| | | | | | | | MGC131894; |
| | | | | | | | HNGC: 16303; GRAF- |
| | | | | | | | 1EF; LENG7; EF1A |
| 841 | 826 | 3460008 | NM_198282.1 | NM_198282 | transmembrane protein | TMEM173 | FLJ38577 |
| | | | | | 173 (TMEM173), | | |
| | | | | | mRNA. | | |
| 842 | 827 | 3460121 | NM_014765.1 | NM_014765 | translocase of outer | TOMM20 | TOM20; MOM19; |
| | | | | | mitochondrial | | KIAA0016; |
| | | | | | membrane 20 homolog | | MGC117367; MAS20 |
| | | | | | (yeast) (TOMM20), | | |
| | | | | | nuclear gene encoding | | |
| | | | | | mitochondrial protein, | | |
| | | | | | mRNA. | | |
| 843 | 828 | 3460132 | NM_152341.2 | NM_152341 | progestin and adipoQ | PAQR4 | FLJ30002 |
| | | | | | receptor family member | | |
| | | | | | IV (PAQR4), mRNA. | | |
| 844 | 829 | 3460201 | NM_015999.2 | NM_015999 | adiponectin receptor 1 | ADIPOR1 | ACDCR1; TESBP1A; |
| | | | | | (ADIPOR1), mRNA. | | CGI-45; PAQR1; |
| | | | | | | | FLJ42464; FLJ25385; |
| | | | | | | | CGI45 |
| 845 | 830 | 3460386 | NM_001497.2 | NM_001497 | UDP-Gal:betaGlcNAc | B4GALT1 | MGC50983; GT1; |
| | | | | | beta 1,4- | | beta4Gal-T1; GTB; |
| | | | | | galactosyltransferase, | | B4GAL-T1; GGTB2; |
| | | | | | polypeptide 1 | | DKFZp686N19253 |
| | | | | | (B4GALT1), mRNA. | | |
| 846 | 831 | 3460424 | NM_212469.1 | NM_212469 | choline kinase alpha | CHKA | CHK; CKI |
| | | | | | (CHKA), transcript | | |
| | | | | | variant 2, mRNA. | | |
| 847 | 832 | 3460441 | NM_003792.2 | NM_003792 | endothelial | EDF1 | EDF-1; MBF1; |
| | | | | | differentiation-related | | MGC9058 |
| | | | | | factor 1 (EDF1), | | |
| | | | | | transcript variant alpha, | | |
| | | | | | mRNA. | | |
| 848 | 833 | 3460451 | NM_014631.2 | NM_014631 | SH3 and PX domains | SH3PXD2A | SH3MD1; FISH |
| | | | | | 2A (SH3PXD2A), | | |
| | | | | | mRNA. | | |
| 849 | 834 | 3460504 | NM_014395.1 | NM_014395 | dual adaptor of | DAPP1 | DKFZp667E0716; |
| | | | | | phosphotyrosine and 3- | | BAM32 |
| | | | | | phosphoinositides | | |
| | | | | | (DAPP1), mRNA. | | |
| 850 | 835 | 3460685 | NM_003937.2 | NM_003937 | kynureninase (L- | KYNU | — |
| | | | | | kynurenine hydrolase) | | |
| | | | | | (KYNU), transcript | | |
| | | | | | variant 1, mRNA. | | |
| 851 | 836 | 3520020 | NM_001037333.1 | NM_001037333 | cytoplasmic FMR1 | CYFIP2 | PIR121 |
| | | | | | interacting protein 2 | | |
| | | | | | (CYFIP2), transcript | | |
| | | | | | variant 1, mRNA. | | |
| 852 | 837 | 3520072 | NM_018135.2 | NM_018135 | mitochondrial ribosomal | MRPS18A | S18bmt; MRPS18-3; |
| | | | | | protein S18A | | HumanS18b; |
| | | | | | (MRPS18A), nuclear | | FLJ10548; MRP-S18-3 |
| | | | | | gene encoding | | |
| | | | | | mitochondrial protein, | | |
| | | | | | mRNA. | | |
| 853 | 838 | 3520309 | NM_031417.2 | NM_031417 | MAP/microtubule | MARK4 | Nbla00650; |
| | | | | | affinity-regulating | | FLJ90097; MARKL1; |
| | | | | | kinase 4 (MARK4), | | KIAA1860 |
| | | | | | mRNA. | | |
| 854 | 839 | 3520370 | NM_138962.2 | NM_138962 | musashi homolog 2 | MSI2 | FLJ36569; MSI2H; |
| | | | | | (Drosophila) (MSI2), | | MGC3245 |
| | | | | | transcript variant 1, | | |
| | | | | | mRNA. | | |
| 855 | 840 | 3520463 | NM_175061.3 | NM_175061 | JAZF zinc finger 1 | JAZF1 | ZNF802; TIP27; |
| | | | | | (JAZF1), mRNA. | | DKFZp761K2222 |
| 856 | 841 | 3520671 | NM_006412.3 | NM_006412 | 1-acylglycerol-3- | AGPAT2 | LPAAB; BSCL1; 1- |
| | | | | | phosphate O- | | AGPAT2; LPAAT- |
| | | | | | acyltransferase 2 | | beta; BSCL |
| | | | | | (lysophosphatidic acid | | |
| | | | | | acyltransferase, beta) | | |
| | | | | | (AGPAT2), transcript | | |
| | | | | | variant 1, mRNA. | | |
| 857 | 842 | 3520689 | NM_018332.3 | NM_018332 | DEAD (Asp-Glu-Ala- | DDX19A | DDX19L; FLJ11126; |
| | | | | | As) box polypeptide | | DDX19-DDX19L; |
| | | | | | 19A (DDX19A), mRNA. | | DKFZp686C21137 |
| 858 | 843 | 3610553 | NM_000181.2 | NM_000181 | glucuronidase, beta | GUSB | MPS7; FLJ39445 |
| | | | | | (GUSB), mRNA. | | |
| 859 | 844 | 3710068 | NM_173701.1 | NM_173701 | tryptophanyl- | WARS | IFI53; IFP53; |
| | | | | | tRNAsynthetase | | GAMMA-2 |
| | | | | | (WARS), transcript | | |
| | | | | | variant 2, mRNA. | | |
| 860 | 845 | 3710243 | NM_024625.3 | NM_024625 | zinc finger CCCH-type, | ZC3HAV1 | FLJ13288; |
| | | | | | antiviral 1 (ZC3HAV1), | | ZC3HDC2; FLB6421; |
| | | | | | transcript variant 2, | | ZAP; |
| | | | | | mRNA. | | DKFZp686H1869; |
| | | | | | | | DKFZp686O19171; |
| | | | | | | | MGC48898; |
| | | | | | | | DKFZp686F2052; |
| | | | | | | | ZC3H2 |
| 861 | 846 | 3710681 | NM_006421.3 | NM_006421 | ADP-ribosylation factor | ARFGEF1 | DKFZP434L057; |
| | | | | | guanine nucleotide- | | BIG1; ARFGEP1; |
| | | | | | exchange factor | | P200; |
| | | | | | 1 (brefeldin A-inhibited) | | D730028O18Rik |
| | | | | | (ARFGEF1), mRNA. | | |
| 862 | 847 | 3780053 | NM_016081.3 | NM_016081 | palladin, cytoskeletal | PALLD | CGI-151; PNCA1; |
| | | | | | associated protein | | KIAA0992; FLJ22190; |
| | | | | | (PALLD), mRNA. | | SIH002; FLJ38193; |
| | | | | | | | FLJ39139 |
| 863 | 848 | 3780674 | NM_032772.3 | NM_032772 | zinc finger protein 503 | ZNF503 | NOLZ-1; MGC2555; |
| | | | | | (ZNF503), mRNA. | | FLJ45745 |
| 864 | 849 | 3800286 | NM_007156.3 | NM_007156 | zinc finger, X-linked, | ZXDA | — |
| | | | | | duplicated A (ZXDA), | | |
| | | | | | mRNA. | | |
| 865 | 851 | 3800392 | NM_001009184.1 | NM_001009184 | glutamate receptor, | GRINA | HNRGW; NMDARA1; |
| | | | | | ionotropic, N-methyl D- | | TMBIM3; MGC99687 |
| | | | | | aspartate-associated | | |
| | | | | | protein 1 (glutamate | | |
| | | | | | binding) (GRINA), | | |
| | | | | | transcript variant 2, | | |
| | | | | | mRNA. | | |
| 866 | 852 | 3800470 | NM_024298.2 | NM_024298 | membrane bound O- | MBOAT7 | hMBOA-7; LPIAT; |
| | | | | | acyltransferase domain | | BB1; LENG4 |
| | | | | | containing 7 | | |
| | | | | | (MBOAT7), mRNA. | | |
| 867 | 853 | 3830735 | NM_016327.2 | NM_016327 | ureidopropionase, beta | UPB1 | BUP1 |
| | | | | | (UPB1), mRNA. | | |
| 868 | 854 | 3840100 | NM_014933.2 | NM_014933 | SEC31 homolog A (S. cerevisiae) | SEC31A | KIAA0905; HSPC334; |
| | | | | | (SEC31A), | | MGC90305; ABP125; |
| | | | | | transcript variant 1, | | SEC31L1; HSPC275; |
| | | | | | mRNA. | | DKFZp686N07171; |
| | | | | | | | ABP130 |
| 869 | 855 | 3840370 | NM_173620.2 | NM_173620 | hexosaminidase | HEXDC | FLJ23825 |
| | | | | | (glycosyl hydrolase | | |
| | | | | | family 20, catalytic | | |
| | | | | | domain) containing | | |
| | | | | | (HEXDC), mRNA. | | |
| 870 | 856 | 3840593 | NM_004510.2 | NM_004510 | SP110 nuclear body | SP110 | FLJ22835; IFI75; |
| | | | | | protein (SP110), | | VODI; IFI41 |
| | | | | | transcript variant b, | | |
| | | | | | mRNA. | | |
| 871 | 857 | 3840717 | NM_016428.2 | NM_016428 | ABI gene family, | ABI3 | SSH3BP3; NESH |
| | | | | | member 3 (ABI3), | | |
| | | | | | mRNA. | | |
| 872 | 858 | 3850053 | NM_006401.2 | NM_006401 | acidic (leucine-rich) | ANP32B | PHAPI2; SSP29; |
| | | | | | nuclear phosphoprotein | | APRIL |
| | | | | | 32 family, member B | | |
| | | | | | (ANP32B), mRNA. | | |
| 873 | 859 | 3850112 | NM_052960.1 | NM_052960 | retinol binding protein 7, | RBP7 | CRBP4; MGC70641; |
| | | | | | cellular (RBP7), mRNA. | | CRBPIV |
| 874 | 860 | 3870242 | NM_018155.1 | NM_018155 | solute carrier family 25, | SLC25A36 | FLJ10618 |
| | | | | | member 36 | | |
| | | | | | (SLC25A36), mRNA. | | |
| 875 | 861 | 3870646 | NM_024065.3 | NM_024065 | phosducin-like 3 | PDCL3 | VIAF1; HTPHLP; |
| | | | | | (PDCL3), mRNA. | | MGC3062 |
| 876 | 862 | 3890017 | NM_001903.2 | NM_001903 | catenin (cadherin- | CTNNA1 | CAP102; FLJ36832 |
| | | | | | associated protein), | | |
| | | | | | alpha 1, 102 kDa | | |
| | | | | | (CTNNA1), mRNA. | | |
| 877 | 863 | 3890138 | NM_181873.2 | NM_181873 | myotubularin related | MTMR11 | CRA; RP11-212K13.1 |
| | | | | | protein 11 (MTMR11), | | |
| | | | | | mRNA. | | |
| 878 | 864 | 3890193 | NM_013943.1 | NM_013943 | chloride intracellular | CLIC4 | p64H1; CLIC4L; H1; |
| | | | | | channel 4 (CLIC4), | | FLJ38640; |
| | | | | | nuclear gene encoding | | DKFZP566G223; |
| | | | | | mitochondrial protein, | | huH1 |
| | | | | | mRNA. | | |
| 879 | 865 | 3890220 | NM_078474.2 | NM_078474 | TM2 domain containing | TM2D3 | BLP2 |
| | | | | | 3 (TM2D3), transcript | | |
| | | | | | variant 1, mRNA. | | |
| 880 | 866 | 3890349 | NM_003542.3 | NM_003542 | histone cluster 1, H4c | HIST1H4C | H4FG; dJ221C16.1; |
| | | | | | (HIST1H4C), mRNA. | | H4/g |
| 881 | 867 | 3890408 | NM_001042576.1 | NM_001042576 | ribosome binding | RRBP1 | DKFZp586A1420; |
| | | | | | protein 1 homolog | | MGC157721; ES130; |
| | | | | | 180 kDa (dog) | | FLJ36146; |
| | | | | | (RRBP1), transcript | | MGC157720; ES/130; |
| | | | | | variant 1, mRNA. | | hES |
| 882 | 868 | 3930561 | NM_002740.5 | NM_002740 | protein kinase C, iota | PRKCI | DXS1179E; |
| | | | | | (PRKCI), mRNA. | | MGC26534; PKCI; |
| | | | | | | | nPKC-iota |
| 883 | 869 | 3930594 | NM_003885.2 | NM_003885 | cyclin-dependent | CDK5R1 | p35nck5a; CDK5P35; |
| | | | | | kinase 5, regulatory | | MGC33831; NCK5A; |
| | | | | | subunit 1 (p35) | | p23; p35; p25; |
| | | | | | (CDK5R1), mRNA. | | CDK5R |
| 884 | 870 | 3940026 | NM_014320.2 | NM_014320 | heme binding protein 2 | HEBP2 | RP3-422G23.1; |
| | | | | | (HEBP2), mRNA. | | KIAA1244; |
| | | | | | | | C6ORF34B; PP23; |
| | | | | | | | SOUL; C6orf34 |
| 885 | 871 | 3940333 | NM_006631.2 | NM_006631 | zinc finger protein 266 | ZNF266 | HZF1 |
| | | | | | (ZNF266), mRNA. | | |
| 886 | 872 | 3940376 | NM_001033578.1 | NM_001033578 | serum/glucocorticoid | SGK3 | SGK2; CISK; |
| | | | | | regulated kinase family, | | DKFZp781N0293; |
| | | | | | member 3 (SGK3), | | SGKL |
| | | | | | transcript variant 3, | | |
| | | | | | mRNA. | | |
| 887 | 873 | 3940438 | NM_000265.4 | NM_000265 | neutrophil cytosolic | NCF1 | NOXO2; SH3PXD1A; |
| | | | | | factor 1, (chronic | | NCF1A; p47phox |
| | | | | | granulomatous | | |
| | | | | | disease, autosomal 1) | | |
| | | | | | (NCF1), mRNA. | | |
| 888 | 874 | 3990482 | NM_004582.2 | NM_004582 | Rabgeranylgeranyltransferase, | RABGGTB | GGTB |
| | | | | | beta subunit | | |
| | | | | | (RABGGTB), mRNA. | | |
| 889 | 875 | 3990546 | NM_025201.3 | NM_025201 | pleckstrin homology | PLEKHO2 | pp9099; PP1628; |
| | | | | | domain containing, | | DKFZp761K2312; |
| | | | | | family O member 2 | | FLJ38884 |
| | | | | | (PLEKHO2), mRNA. | | |
| 890 | 876 | 3990598 | NM_006442.2 | NM_006442 | DR1-associated protein | DRAP1 | NC2-alpha |
| | | | | | 1 (negative cofactor 2 | | |
| | | | | | alpha) (DRAP1), | | |
| | | | | | mRNA. | | |
| 891 | 877 | 4010020 | NM_172373.2 | NM_172373 | E74-like factor 1 (ets | ELF1 | — |
| | | | | | domain transcription | | |
| | | | | | factor) (ELF1), mRNA. | | |
| 892 | 878 | 4010452 | NM_153811.1 | NM_153811 | solute carrier family 38, | SLC38A6 | NAT-1; MGC102697 |
| | | | | | member 6 (SLC38A6), | | |
| | | | | | mRNA. | | |
| 893 | 879 | 4040022 | NM_175862.2 | NM_175862 | CD86 antigen (CD28 | CD86 | B7-2; B70; LAB72; |
| | | | | | antigen ligand 2, B7-2 | | MGC34413; |
| | | | | | antigen) (CD86), | | CD28LG2 |
| | | | | | transcript variant 1, | | |
| | | | | | mRNA. | | |
| 894 | 880 | 4040398 | NM_022440.1 | NM_022440 | mal, T-cell | MAL | — |
| | | | | | differentiation protein | | |
| | | | | | (MAL), transcript variant | | |
| | | | | | d, mRNA. | | |
| 895 | 881 | 4050161 | NM_005819.4 | NM_005819 | syntaxin 6 (STX6), | STX6 | — |
| | | | | | mRNA. | | |
| 896 | 882 | 4050202 | NM_194448.1 | NM_194448 | C-type lectin domain | CLEC4A | DCIR; DDB27; LLIR; |
| | | | | | family 4, member A | | HDCGC13P; |
| | | | | | (CLEC4A), transcript | | CLECSF6 |
| | | | | | variant 4, mRNA. | | |
| 897 | 883 | 4050491 | NM_003196.1 | NM_003196 | transcription elongation | TCEA3 | TFIIS.H; TFIIS |
| | | | | | factor A (SII), 3 | | |
| | | | | | (TCEA3), mRNA. | | |
| 898 | 884 | 4050600 | NM_001039935.1 | NM_001039935 | ankyrin repeat domain | ANKRD55 | FLJ11795; |
| | | | | | 55 (ANKRD55), | | MGC126014; |
| | | | | | transcript variant 2, | | MGC126013 |
| | | | | | mRNA. | | |
| 899 | 885 | 4060358 | NM_005502.2 | NM_005502 | ATP-binding cassette, | ABCA1 | CERP; ABC-1; |
| | | | | | sub-family A (ABC1), | | MGC164864; |
| | | | | | member 1 (ABCA1), | | MGC165011; |
| | | | | | mRNA. | | HDLDT1; ABC1; |
| | | | | | | | TGD; FLJ14958 |
| 900 | 886 | 4070017 | NM_002539.1 | NM_002539 | omithine decarboxylase | ODC1 | — |
| | | | | | 1 (ODC1), mRNA. | | |
| 901 | 887 | 4070300 | NM_052839.2 | NM_052839 | pannexin 2 (PANX2), | PANX2 | hPANX2; |
| | | | | | mRNA. | | MGC119432 |
| 902 | 888 | 4070367 | NM_145645.2 | NM_145645 | NOL1/NOP2/Sun | NSUN5B | WBSCR20B; |
| | | | | | domain family, member | | MGC129801 |
| | | | | | 5B (NSUN5B), | | |
| | | | | | transcript variant 2, | | |
| | | | | | mRNA. | | |
| 903 | 889 | 4070538 | NM_018319.3 | NM_018319 | tyrosyl-DNA | TDP1 | FLJ11090; |
| | | | | | phosphodiesterase 1 | | MGC104252 |
| | | | | | (TDP1), transcript | | |
| | | | | | variant 1, mRNA. | | |
| 904 | 890 | 4120411 | NM_004723.2 | NM_004723 | rho/rac guanine | ARHGEF2 | P40; GEF; LFP40; |
| | | | | | nucleotide exchange | | DKFZp547L106; |
| | | | | | factor (GEF) 2 | | GEF-H1; |
| | | | | | (ARHGEF2), mRNA. | | DKFZp547P1516; |
| | | | | | | | GEFH1; KIAA0651 |
| 905 | 891 | 4120689 | NM_002005.2 | NM_002005 | feline sarcoma | FES | FPS |
| | | | | | oncogene (FES), | | |
| | | | | | mRNA. | | |
| 906 | 892 | 4150017 | NM_178422.4 | NM_178422 | progestin and adipoQ | PAQR7 | MPRA; mSR |
| | | | | | receptor family member | | |
| | | | | | VII (PAQR7), mRNA. | | |
| 907 | 893 | 4150048 | NM_001444.1 | NM_001444 | fatty acid binding | FABP5 | PA-FABP; PAFABP; |
| | | | | | protein 5 (psoriasis- | | E-FABP; EFABP |
| | | | | | associated) (FABP5), | | |
| | | | | | mRNA. | | |
| 908 | 894 | 4150136 | NM_003952.2 | NM_003952 | ribosomal protein S6 | RPS6KB2 | p70S6Kb; P70-beta-2; |
| | | | | | kinase, 70 kDa, | | S6K-beta2; p70(S6K)- |
| | | | | | polypeptide 2 | | beta; P70-beta-1; |
| | | | | | (RPS6KB2), mRNA. | | KLS; S6K2; SRK; |
| | | | | | | | STK14B; P70-beta |
| 909 | 895 | 4150189 | NM_001912.3 | NM_001912 | cathepsin L1 (CTSL1), | CTSL1 | MEP; CATL; |
| | | | | | transcript variant 1, | | FLJ31037; CTSL |
| | | | | | mRNA. | | |
| 910 | 896 | 4150458 | NM_002947.3 | NM_002947 | replication protein A3, | RPA3 | REPA3 |
| | | | | | 14 kDa (RPA3), mRNA. | | |
| 911 | 897 | 4150500 | NM_004371.3 | NM_004371 | coatomer protein | COPA | FLJ26320; HEP-COP |
| | | | | | complex, subunit alpha | | |
| | | | | | (COPA), transcript | | |
| | | | | | variant 2, mRNA. | | |
| 912 | 898 | 4150687 | NM_004546.2 | NM_004546 | NADH dehydrogenase | NDUFB2 | AGGG; MGC70788; |
| | | | | | (ubiquinone) 1 beta | | CI-AGGG |
| | | | | | subcomplex, 2, 8 kDa | | |
| | | | | | (NDUFB2), nuclear | | |
| | | | | | gene encoding | | |
| | | | | | mitochondrial protein, | | |
| | | | | | mRNA. | | |
| 913 | 899 | 4180079 | NM_031476.2 | NM_031476 | cysteine-rich secretory | CRISPLD2 | DKFZP434B044; |
| | | | | | protein LCCL domain | | MGC74865; |
| | | | | | containing 2 | | CRISP11; LCRISP2 |
| | | | | | (CRISPLD2), mRNA. | | |
| 914 | 900 | 4180259 | NM_018921.2 | NM_018921 | protocadherin gamma | PCDHGA9 | PCDH-GAMMA-A9 |
| | | | | | subfamily A, 9 | | |
| | | | | | (PCDHGA9), transcript | | |
| | | | | | variant 1, mRNA. | | |
| 915 | 901 | 4180468 | NM_004729.3 | NM_004729 | zinc finger, BED-type | ZBED1 | KIAA0785; TRAMP; |
| | | | | | containing 1 (ZBED1), | | ALTE; DREF |
| | | | | | mRNA. | | |
| 916 | 902 | 4200019 | NM_001085458.1 | NM_001085458 | catenin (cadherin- | CTNND1 | CAS; P120CAS; |
| | | | | | associated protein), | | p120; P120CTN; |
| | | | | | delta 1 (CTNND1), | | CTNND; KIAA0384 |
| | | | | | transcript variant 1, | | |
| | | | | | mRNA. | | |
| 917 | 903 | 4200110 | NM_138408.2 | NM_138408 | general transcription | GTF3C6 | C6orf51; TFIIIC35; |
| | | | | | factor IIIC, polypeptide | | bA397G5.3 |
| | | | | | 6, alpha 35 kDa | | |
| | | | | | (GTF3C6), mRNA. | | |
| 918 | 904 | 4200176 | NM_004107.3 | NM_004107 | Fc fragment of IgG, | FCGRT | alpha-chain; FCRN |
| | | | | | receptor, transporter, | | |
| | | | | | alpha (FCGRT), | | |
| | | | | | mRNA. | | |
| 919 | 905 | 4200725 | NR_001298.1 | NR_001298 | major histocompatibility | HLA-DRB6 | — |
| | | | | | complex, class II, DR | | |
| | | | | | beta 6 (pseudogene) | | |
| | | | | | (HLA-DRB6) on | | |
| | | | | | chromosome 6. | | |
| 920 | 906 | 4200743 | NM_015175.1 | NM_015175 | neurobeachin-like 2 | NBEAL2 | KIAA0540 |
| | | | | | (NBEAL2), mRNA. | | |
| 921 | 907 | 4210044 | NR_000024.2 | NR_000024 | small nucleolar RNA, | SNORD46 | U46; RNU40; RNU46; |
| | | | | | C/D box 46 | | U40 |
| | | | | | (SNORD46) on | | |
| | | | | | chromosome 1. | | |
| 922 | 908 | 4210066 | NM_017702.2 | NM_017702 | differentially expressed | DEF8 | MGC104349; |
| | | | | | in FDCP 8 homolog | | FLJ20186 |
| | | | | | (mouse) (DEF8), | | |
| | | | | | transcript variant 2, | | |
| | | | | | mRNA. | | |
| 923 | 909 | 4210431 | NM_003135.1 | NM_003135 | signal recognition | SRP19 | — |
| | | | | | particle 19 kDa | | |
| | | | | | (SRP19), mRNA. | | |
| 924 | 910 | 4210647 | NR_000033.2 | NR_000033 | estrogen-related | ESRRAP2 | ESTRRA |
| | | | | | receptor alpha | | |
| | | | | | pseudogene 2 | | |
| | | | | | (ESRRAP2) on | | |
| | | | | | chromosome 13. | | |
| 925 | 911 | 4220168 | NM_001024074.1 | NM_001024074 | histamine N- | HNMT | HMT; HNMT-S2; |
| | | | | | methyltransferase | | HNMT-S1 |
| | | | | | (HNMT), transcript | | |
| | | | | | variant 2, mRNA. | | |
| 926 | 912 | 4220187 | NM_003494.2 | NM_003494 | dysferlin, limb girdle | DYSF | LGMD2B; FER1L1; |
| | | | | | muscular dystrophy 2B | | FLJ90168; FLJ00175 |
| | | | | | (autosomal recessive) | | |
| | | | | | (DYSF), mRNA. | | |
| 927 | 913 | 4220259 | NM_001336.2 | NM_001336 | cathepsin Z (CTSZ), | CTSZ | CTSX |
| | | | | | mRNA. | | |
| 928 | 914 | 4220603 | NM_003120.2 | NM_003120 | spleen focus forming | SPI1 | SPI-A; SFPI1; PU.1; |
| | | | | | virus (SFFV) proviral | | OF; SPI-1 |
| | | | | | integration oncogene | | |
| | | | | | spi1 (SPI1), transcript | | |
| | | | | | variant 2, mRNA. | | |
| 929 | 915 | 4230168 | NM_005908.3 | NM_005908 | mannosidase, beta A, | MANBA | MANB1 |
| | | | | | lysosomal (MANBA), | | |
| | | | | | mRNA. | | |
| 930 | 916 | 4230520 | NM_003746.1 | NM_003746 | dynein, cytoplasmic, | DNCL1 | PIN; MGC126138; |
| | | | | | light polypeptide 1 | | hdlc1; DLC1; |
| | | | | | (DNCL1), mRNA. | | MGC126137; LC8; |
| | | | | | | | DLC8 |
| 931 | 917 | 4230554 | NM_015523.2 | NM_015523 | REX2, RNA | REXO2 | CGI-114; |
| | | | | | exonuclease 2 homolog | | MGC111570; |
| | | | | | (S. cerevisiae) | | DKFZP566E144; |
| | | | | | (REXO2), mRNA. | | RFN; SFN |
| 932 | 918 | 4230626 | NM_006254.3 | NM_006254 | protein kinase C, delta | PRKCD | MAY1; MGC49908; |
| | | | | | (PRKCD), transcript | | nPKC-delta |
| | | | | | variant 1, mRNA. | | |
| 933 | 919 | 4230669 | NM_006714.2 | NM_006714 | sphingomyelinphospho | SMPDL3A | yR36GH4.1; |
| | | | | | diesterase, acid-like 3A | | FLJ20177; ASM3A; |
| | | | | | (SMPDL3A), mRNA. | | ASML3a |
| 934 | 920 | 4230671 | NM_175077.1 | NM_175077 | Src-like-adaptor 2 | SLA2 | FLJ21992; |
| | | | | | (SLA2), transcript | | MGC49845; SLAP2; |
| | | | | | variant 2, mRNA. | | C20orf156; SLAP-2 |
| 935 | 921 | 4230673 | NM_000839.2 | NM_000839 | glutamate receptor, | GRM2 | mGlu2; GPRC1B; |
| | | | | | metabotropic 2 | | MGLUR2; GLUR2 |
| | | | | | (GRM2), mRNA. | | |
| 936 | 922 | 4250082 | NM_032221.3 | NM_032221 | chromodomain helicase | CHD6 | CHD5; KIAA1335; |
| | | | | | DNA binding protein 6 | | RIGB |
| | | | | | (CHD6), mRNA. | | |
| 937 | 923 | 4250327 | NM_175744.4 | NM_175744 | ras homolog gene | RHOC | H9; ARHC; |
| | | | | | family, member C | | MGC61427; |
| | | | | | (RHOC), transcript | | MGC1448; RHOH9; |
| | | | | | variant 1, mRNA. | | ARH9 |
| 938 | 924 | 4250343 | NM_006241.3 | NM_006241 | protein phosphatase 1, | PPP1R2 | MGC87148; IPP2 |
| | | | | | regulatory (inhibitor) | | |
| | | | | | subunit 2 (PPP1R2), | | |
| | | | | | mRNA. | | |
| 939 | 925 | 4250630 | NM_016523.1 | NM_016523 | killer cell lectin-like | KLRF1 | MGC119907; |
| | | | | | receptor subfamily F, | | CLEC5C; |
| | | | | | member 1 (KLRF1), | | MGC119908; |
| | | | | | mRNA. | | MGC119909 |
| 940 | 926 | 4250735 | NM_004843.2 | NM_004843 | interleukin 27 receptor, | IL27RA | zcytor1; WSX1; |
| | | | | | alpha (IL27RA), mRNA. | | IL27R; TCCR; CRL1 |
| 941 | 927 | 4260019 | NM_016645.2 | NM_016645 | neugrin, neurite | NGRN | NEUGRIN; DSC92 |
| | | | | | outgrowth associated | | |
| | | | | | (NGRN), transcript | | |
| | | | | | variant 1, mRNA. | | |
| 942 | 928 | 4260075 | NM_014463.1 | NM_014463 | LSM3 homolog, U6 | LSM3 | USS2; YLR438C; |
| | | | | | small nuclear RNA | | SMX4 |
| | | | | | associated (S. cerevisiae) | | |
| | | | | | (LSM3), | | |
| | | | | | mRNA. | | |
| 943 | 929 | 4260152 | NM_032174.4 | NM_032174 | translocase of outer | TOMM40L | RP11-297K8.10; |
| | | | | | mitochondrial | | TOMM40B; FLJ12770 |
| | | | | | membrane 40 homolog | | |
| | | | | | (yeast)-like | | |
| | | | | | (TOMM40L), nuclear | | |
| | | | | | gene encoding | | |
| | | | | | mitochondrial protein, | | |
| | | | | | mRNA. | | |
| 944 | 930 | 4260187 | NM_181715.1 | NM_181715 | CREB regulated | CRTC2 | RP11-422P24.6; |
| | | | | | transcription coactivator | | TORC2 |
| | | | | | 2 (CRTC2), mRNA. | | |
| 945 | 931 | 4260373 | NM_001098631.1 | NM_001098631 | interferon regulatory | IRF5 | — |
| | | | | | factor 5 (IRF5), | | |
| | | | | | transcript variant 7, | | |
| | | | | | mRNA. | | |
| 946 | 932 | 4260386 | NM_145918.2 | NM_145918 | cathepsin L1 (CTSL1), | CTSL1 | MEP; CATL; |
| | | | | | transcript variant 2, | | FLJ31037; CTSL |
| | | | | | mRNA. | | |
| 947 | 933 | 4260504 | NM_020468.2 | NM_020468 | sorting nexin 14 | SNX14 | MGC13217; RP11- |
| | | | | | (SNX14), transcript | | 321N4.2; RGS-PX2 |
| | | | | | variant 2, mRNA. | | |
| 948 | 934 | 4260551 | NM_001018060.1 | NM_001018060 | apoptosis-inducing | AIFM3 | FLJ30473; AIFL |
| | | | | | factor, mitochondrion- | | |
| | | | | | associated, 3 (AIFM3), | | |
| | | | | | nuclear gene encoding | | |
| | | | | | mitochondrial protein, | | |
| | | | | | transcript variant 2, | | |
| | | | | | mRNA. | | |
| 949 | 935 | 4260593 | NM_001001560.1 | NM_001001560 | golgi associated, | GGA1 | — |
| | | | | | gamma adaptin ear | | |
| | | | | | containing, ARF binding | | |
| | | | | | protein 1 (GGA1), | | |
| | | | | | transcript variant 2, | | |
| | | | | | mRNA. | | |
| 950 | 936 | 4260735 | NM_021159.3 | NM_021159 | RAP1, GTP-GDP | RAP1GDS1 | MGC118861; |
| | | | | | dissociation stimulator 1 | | MGC118859; GDS1 |
| | | | | | (RAP1GDS1), mRNA. | | |
| 951 | 937 | 4280253 | NM_017906.2 | NM_017906 | PAK1 interacting | PAK1IP1 | hPIP1; RP11- |
| | | | | | protein 1 (PAK1IP1), | | 421M1.5; PIP1; |
| | | | | | mRNA. | | MAK11; FLJ20624; |
| | | | | | | | bA421M1.5; WDR84 |
| 952 | 938 | 4280348 | NM_178841.2 | NM_178841 | ring finger protein 166 | RNF166 | MGC14381; |
| | | | | | (RNF166), mRNA. | | MGC2647 |
| 953 | 939 | 4280435 | NM_205839.1 | NM_205839 | leukocyte specific | LST1 | D6S49E; LST-1; |
| | | | | | transcript 1 (LST1), | | B144; MGC119007; |
| | | | | | transcript variant 4, | | MGC119006 |
| | | | | | mRNA. | | |
| 954 | 940 | 4280603 | NM_006527.2 | NM_006527 | stem-loop binding | SLBP | HBP |
| | | | | | protein (SLBP), mRNA. | | |
| 955 | 941 | 4290368 | NM_024430.2 | NM_024430 | proline-serine-threonine | PSTPIP2 | MGC34175; MAYP |
| | | | | | phosphatase interacting | | |
| | | | | | protein 2 (PSTPIP2), | | |
| | | | | | mRNA. | | |
| 956 | 942 | 4390079 | NM_022136.3 | NM_022136 | SAM domain, SH3 | SAMSN1 | NASH1; HACS1; |
| | | | | | domain and nuclear | | SASH2; SH3D6B |
| | | | | | localization signals 1 | | |
| | | | | | (SAMSN1), mRNA. | | |
| 957 | 943 | 4390546 | NM_014886.3 | NM_014886 | TGF beta-inducible | TINP1 | NSA2; HCL-G1; YR- |
| | | | | | nuclear protein 1 | | 29; CDK105 |
| | | | | | (TINP1), mRNA. | | |
| 958 | 944 | 4390619 | NM_001042678.1 | NM_001042678 | ras homolog gene | RHOC | H9; ARHC; |
| | | | | | family, member C | | MGC61427; |
| | | | | | (RHOC), transcript | | MGC1448; RHOH9; |
| | | | | | variant 2, mRNA. | | ARH9 |
| 959 | 945 | 4480224 | NM_021203.2 | NM_021203 | signal recognition | SRPRB | APMCF1 |
| | | | | | particle receptor, B | | |
| | | | | | subunit (SRPRB), | | |
| | | | | | mRNA. | | |
| 960 | 946 | 4490176 | NM_002922.3 | NM_002922 | regulator of G-protein | RGS1 | BL34; IR20; IER1; |
| | | | | | signaling 1 (RGS1), | | 1R20 |
| | | | | | mRNA. | | |
| 961 | 947 | 4490500 | NM_000887.3 | NM_000887 | integrin, alpha X | ITGAX | CD11C |
| | | | | | (complement | | |
| | | | | | component 3 receptor 4 | | |
| | | | | | subunit) (ITGAX), | | |
| | | | | | mRNA. | | |
| 962 | 948 | 4540228 | NM_139018.2 | NM_139018 | CD300 molecule-like | CD300LF | IREM1; IgSF13; |
| | | | | | family member f | | NKIR; CLM1; CD300f |
| | | | | | (CD300LF), mRNA. | | |
| 963 | 949 | 4540349 | NM_021177.3 | NM_021177 | LSM2 homolog, U6 | LSM2 | G7b; snRNP; |
| | | | | | small nuclear RNA | | C6orf28; YBL026W |
| | | | | | associated (S. cerevisiae) | | |
| | | | | | (LSM2), | | |
| | | | | | mRNA. | | |
| 964 | 950 | 4560435 | NM_024028.2 | NM_024028 | prenylcysteine oxidase | PCYOX1L | MGC3265 |
| | | | | | 1 like (PCYOX1L), | | |
| | | | | | mRNA. | | |
| 965 | 951 | 4560743 | NM_005816.4 | NM_005816 | CD96 molecule | CD96 | MGC22596; |
| | | | | | (CD96), transcript | | DKFZp667E2122; |
| | | | | | variant 2, mRNA. | | TACTILE |
| 966 | 952 | 4570008 | NM_005107.2 | NM_005107 | endonuclease G-like 1 | ENDOGL1 | ENGL; MGC125945; |
| | | | | | (ENDOGL1), mRNA. | | ENDOGL2; |
| | | | | | | | MGC125944; ENGL- |
| | | | | | | | B; ENGL-a |
| 967 | 953 | 4570433 | NM_016222.2 | NM_016222 | DEAD (Asp-Glu-Ala- | DDX41 | MGC8828; ABS |
| | | | | | Asp) box polypeptide | | |
| | | | | | 41 (DDX41), mRNA. | | |
| 968 | 954 | 4570438 | NM_001251.2 | NM_001251 | CD68 molecule | CD68 | DKFZp686M18236; |
| | | | | | (CD68), transcript | | SCARD1; GP110 |
| | | | | | variant 1, mRNA. | | |
| 969 | 955 | 4570494 | NM_012227.1 | NM_012227 | GTP binding protein 6 | GTPBP6 | FLJ90047; FLJ22809; |
| | | | | | (putative) (GTPBP6), | | PGPL; FLJ20977 |
| | | | | | mRNA. | | |
| 970 | 956 | 4590026 | NM_000884.2 | NM_000884 | IMP (inosine | IMPDH2 | IMPD2; IMPDH-II |
| | | | | | monophosphate) | | |
| | | | | | dehydrogenase 2 | | |
| | | | | | (IMPDH2), mRNA. | | |
| 971 | 957 | 4590110 | NM_006640.3 | NM_006640 | septin 9 (SEPT9), | SEPT9 | AF17q25; SINT1; |
| | | | | | mRNA. | | PNUTL4; SeptD1; |
| | | | | | | | MSF; NAPB; |
| | | | | | | | KIAA0991; MSF1 |
| 972 | 958 | 4590154 | NM_013373.2 | NM_013373 | zinc finger, DHHC-type | ZDHHC8 | ZNF378; ZDHHCL1 |
| | | | | | containing 8 | | |
| | | | | | (ZDHHC8), mRNA. | | |
| 973 | 959 | 4590477 | NM_001042600.1 | NM_001042600 | mitogen-activated | MAP4K1 | HPK1 |
| | | | | | protein kinase | | |
| | | | | | kinasekinasekinase 1 | | |
| | | | | | (MAP4K1), transcript | | |
| | | | | | variant 1, mRNA. | | |
| 974 | 960 | 4590521 | NM_004541.2 | NM_004541 | NADH dehydrogenase | NDUFA1 | ZNF183; MWFE; CI- |
| | | | | | (ubiquinone) 1 alpha | | MWFE |
| | | | | | subcomplex, 1, 7.5 kDa | | |
| | | | | | (NDUFA1), nuclear | | |
| | | | | | gene encoding | | |
| | | | | | mitochondrial protein, | | |
| | | | | | mRNA. | | |
| 975 | 961 | 4610044 | NM_006411.2 | NM_006411 | 1-acylglycerol-3- | AGPAT1 | MGC5423; |
| | | | | | phosphate O- | | MGC4007; LPAAT- |
| | | | | | acyltransferase 1 | | alpha; 1-AGPAT1; |
| | | | | | (lysophosphatidic acid | | G15; LPAATA |
| | | | | | acyltransferase, alpha) | | |
| | | | | | (AGPAT1), transcript | | |
| | | | | | variant 1, mRNA. | | |
| 976 | 962 | 4610138 | NM_006135.1 | NM_006135 | capping protein (actin | CAPZA1 | CAPPA1; CAPZ; |
| | | | | | filament) muscle Z-line, | | CAZ1 |
| | | | | | alpha 1 (CAPZA1), | | |
| | | | | | mRNA. | | |
| 977 | 963 | 4610201 | NR_002327.1 | NR_002327 | small nucleolar RNA, | SNORA10 | ACA10 |
| | | | | | H/ACA box 10 | | |
| | | | | | (SNORA10) on | | |
| | | | | | chromosome 16. | | |
| 978 | 964 | 4610220 | NM_001040196.1 | NM_001040196 | angiotensin II receptor- | AGTRAP | MGC29646; ATRAP |
| | | | | | associated protein | | |
| | | | | | (AGTRAP), transcript | | |
| | | | | | variant 4, mRNA. | | |
| 979 | 965 | 4610414 | NM_032796.2 | NM_032796 | synapse associated | SYAP1 | FLJ14495; |
| | | | | | protein 1, SAP47 | | DKFZp686K221; |
| | | | | | homolog (Drosophila) | | PRO3113; FLJ44185 |
| | | | | | (SYAP1), mRNA. | | |
| 980 | 966 | 4610674 | NM_007047.3 | NM_007047 | butyrophilin, subfamily | BTN3A2 | BT3.3; BT3.2; BTF4 |
| | | | | | 3, member A2 | | |
| | | | | | (BTN3A2), mRNA. | | |
| 981 | 967 | 4610753 | NM_000118.1 | NM_000118 | endoglin (Osler-Rendu- | ENG | ORW; CD105; HHT1; |
| | | | | | Weber syndrome 1) | | END; FLJ41744; |
| | | | | | (ENG), mRNA. | | ORW1 |
| 982 | 968 | 4640064 | NM_002342.1 | NM_002342 | lymphotoxin beta | LTBR | TNF-R-III; TNFRSF3; |
| | | | | | receptor (TNFR | | TNFR-RP; LT-BETA- |
| | | | | | superfamily, member 3) | | R; TNFCR; TNFR2- |
| | | | | | (LTBR), mRNA. | | RP; CD18; D12S370 |
| 983 | 969 | 4640333 | NM_003427.3 | NM_003427 | zinc finger protein 76 | ZNF76 | D6S229E; Zfp523; |
| | | | | | (expressed in testis) | | ZNF523 |
| | | | | | (ZNF76), mRNA. | | |
| 984 | 970 | 4640343 | NM_205840.1 | NM_205840 | leukocyte specific | LST1 | D6S49E; LST-1; |
| | | | | | transcript 1 (LST1), | | B144; MGC119007; |
| | | | | | transcript variant 5, | | MGC119006 |
| | | | | | mRNA. | | |
| 985 | 971 | 4640392 | NM_005937.3 | NM_005937 | myeloid/lymphoid or | MLLT6 | AF17; FLJ23480 |
| | | | | | mixed-lineage leukemia | | |
| | | | | | (trithorax homolog, | | |
| | | | | | Drosophila); | | |
| | | | | | translocated to, 6 | | |
| | | | | | (MLLT6), mRNA. | | |
| 986 | 972 | 4670750 | NM_001065.2 | NM_001065 | tumor necrosis factor | TNFRSF1A | TNFR1; p60; |
| | | | | | receptor superfamily, | | CD120a; p55-R; |
| | | | | | member 1A | | TNFR60; TNF-R55; |
| | | | | | (TNFRSF1A), mRNA. | | TNFAR; TBP1; TNF- |
| | | | | | | | R-I; TNFR55; FPF; |
| | | | | | | | TNF-R; p55; |
| | | | | | | | MGC19588 |
| 987 | 973 | 4730086 | NM_024745.2 | NM_024745 | SHC SH2-domain | SHCBP1 | FLJ22009; |
| | | | | | binding protein 1 | | MGC26900 |
| | | | | | (SHCBP1), mRNA. | | |
| 988 | 974 | 4730349 | NM_022572.2 | NM_022572 | paroxysmal | PNKD | FKSG19; KIPP1184; |
| | | | | | nonkinesiogenic | | DKFZp564N1362; |
| | | | | | dyskinesia (PNKD), | | MR1; FPD1; |
| | | | | | transcript variant 2, | | TAHCCP2; |
| | | | | | mRNA. | | MGC31943; DYT8; |
| | | | | | | | KIAA1184; BRP17; |
| | | | | | | | MR-1; PDC |
| 989 | 975 | 4730743 | NM_007108.2 | NM_007108 | transcription elongation | TCEB2 | SIII |
| | | | | | factor B (SIII), | | |
| | | | | | polypeptide 2 (18 kDa, | | |
| | | | | | elongin B) (TCEB2), | | |
| | | | | | transcript variant 1, | | |
| | | | | | mRNA. | | |
| 990 | 976 | 4760112 | NM_001003714.1 | NM_001003714 | ATP synthase, H+ | ATP5J2 | ATP5JL |
| | | | | | transporting, | | |
| | | | | | mitochondrial F0 | | |
| | | | | | complex, subunit F2 | | |
| | | | | | (ATP5J2), nuclear gene | | |
| | | | | | encoding mitochondrial | | |
| | | | | | protein, transcript | | |
| | | | | | variant 3, mRNA. | | |
| 991 | 977 | 4760364 | NM_012276.3 | NM_012276 | leukocyte | LILRA4 | ILT7; CD85g; |
| | | | | | immunoglobulin-like | | MGC129598; |
| | | | | | receptor, subfamily A | | MGC129597 |
| | | | | | (with TM domain), | | |
| | | | | | member 4 (LILRA4), | | |
| | | | | | mRNA. | | |
| 992 | 978 | 4780136 | NM_016406.1 | NM_016406 | ubiquitin-fold modifier | UFC1 | HSPC155 |
| | | | | | conjugating enzyme 1 | | |
| | | | | | (UFC1), mRNA. | | |
| 993 | 979 | 4780524 | NM_016324.2 | NM_016324 | zinc finger protein 274 | ZNF274 | ZKSCAN19; |
| | | | | | (ZNF274), transcript | | DKFZp686K08243; |
| | | | | | variant ZNF274b, | | FLJ37843; HFB101; |
| | | | | | mRNA. | | ZF2 |
| 994 | 980 | 4780612 | NM_030930.2 | NM_030930 | unc-93 homolog B1 (C. elegans) | UNC93B1 | UNC93B; UNC93; |
| | | | | | (UNC93B1), | | MGC126617 |
| | | | | | mRNA. | | |
| 995 | 981 | 4810020 | NM_002664.1 | NM_002664 | pleckstrin (PLEK), | PLEK | P47; FLJ27168 |
| | | | | | mRNA. | | |
| 996 | 982 | 4810435 | NM_005506.2 | NM_005506 | scavenger receptor | SCARB2 | CD36L2; HLGP85; |
| | | | | | class B, member 2 | | SR-BII; LIMPII |
| | | | | | (SCARB2), mRNA. | | |
| 997 | 983 | 4810615 | NM_014655.1 | NM_014655 | solute carrier family 25, | SLC25A44 | FLJ90431; KIAA0446; |
| | | | | | member 44 | | RP11-54H19.3 |
| | | | | | (SLC25A44), mRNA. | | |
| 998 | 984 | 4810674 | NM_019006.2 | NM_019006 | zinc finger, AN1-type | ZFAND6 | ZA20D3; AWP1; |
| | | | | | domain 6 (ZFAND6), | | ZFAND5B |
| | | | | | mRNA. | | |
| 999 | 985 | 4830100 | NM_007355.2 | NM_007355 | heat shock protein | HSP90AB1 | HSP90-BETA; |
| | | | | | 90 kDa alpha | | HSPC2; FLJ26984; |
| | | | | | (cytosolic), class B | | D6S182; HSP90B; |
| | | | | | member 1 | | HSPCB |
| | | | | | (HSP90AB1), mRNA. | | |
| 1000 | 986 | 4830632 | NM_003982.2 | NM_003982 | solute carrier family 7 | SLC7A7 | LAT3; y+LAT-1; LPI; |
| | | | | | (cationic amino acid | | Y+LAT1 |
| | | | | | transporter, y+ system), | | |
| | | | | | member 7 (SLC7A7), | | |
| | | | | | mRNA. | | |
| 1001 | 987 | 4850164 | NM_005085.2 | NM_005085 | nucleoporin 214 kDa | NUP214 | N214; MGC104525; |
| | | | | | (NUP214), mRNA. | | CAIN; D9S46E; CAN |
| 1002 | 988 | 4850168 | NM_003059.2 | NM_003059 | solute carrier family 22 | SLC22A4 | OCTN1; MGC40524; |
| | | | | | (organic | | MGC34546 |
| | | | | | cation/ergothioneine | | |
| | | | | | transporter), member 4 | | |
| | | | | | (SLC22A4), mRNA. | | |
| 1003 | 989 | 4850270 | NM_003417.3 | NM_003417 | zinc finger protein 264 | ZNF264 | — |
| | | | | | (ZNF264), mRNA. | | |
| 1004 | 990 | 4850301 | NM_012232.3 | NM_012232 | polymerase I and | PTRF | FKSG13 |
| | | | | | transcript release factor | | |
| | | | | | (PTRF), mRNA. | | |
| 1005 | 991 | 4860050 | NM_153047.1 | NM_153047 | FYN oncogene related | FYN | MGC45350; SYN; |
| | | | | | to SRC, FGR, YES | | SLK |
| | | | | | (FYN), transcript variant | | |
| | | | | | 2, mRNA. | | |
| 1006 | 992 | 4860224 | NM_004184.3 | NM_004184 | tryptophanyl- | WARS | IFI53; IFP53; |
| | | | | | tRNAsynthetase | | GAMMA-2 |
| | | | | | (WARS), transcript | | |
| | | | | | variant 1, mRNA. | | |
| 1007 | 993 | 4860286 | NM_018955.2 | NM_018955 | ubiquitin B (UBB), | UBB | FLJ25987; MGC8385 |
| | | | | | mRNA. | | |
| 1008 | 994 | 4860367 | NM_032166.2 | NM_032166 | ATR interacting protein | ATRIP | MGC20625; ATRIP; |
| | | | | | (ATRIP), transcript | | DKFZp762J2115; |
| | | | | | variant 2, mRNA. | | DRN3; FLJ12343; |
| | | | | | | | MGC21482; |
| | | | | | | | DKFZp434J0310; |
| | | | | | | | MGC26740; AGS1 |
| 1009 | 995 | 4860707 | NM_182608.2 | NM_182608 | ankyrin repeat domain | ANKRD33 | DKFZp686O1689; |
| | | | | | 33 (ANKRD33), mRNA. | | C12orf7 |
| 1010 | 996 | 4860753 | NM_080914.1 | NM_080914 | asialoglycoprotein | ASGR2 | L-H2; CLEC4H2; |
| | | | | | receptor 2 (ASGR2), | | Hs.1259; ASGP-R |
| | | | | | transcript variant 3, | | |
| | | | | | mRNA. | | |
| 1011 | 997 | 4860762 | NM_003866.1 | NM_003866 | inositol polyphosphate- | INPP4B | MGC132014 |
| | | | | | 4-phosphatase, type II, | | |
| | | | | | 105 kDa (INPP4B), | | |
| | | | | | mRNA. | | |
| 1012 | 998 | 4880168 | NM_004339.2 | NM_004339 | pituitary tumor- | PTTG1IP | C21orf3; C21orf1; |
| | | | | | transforming 1 | | PBF |
| | | | | | interacting protein | | |
| | | | | | (PTTG1IP), mRNA. | | |
| 1013 | 999 | 4880288 | NM_024527.4 | NM_024527 | abhydrolase domain | ABHD8 | FLJ11743; |
| | | | | | containing 8 (ABHD8), | | MGC14280; |
| | | | | | mRNA. | | MGC2512 |
| 1014 | 1000 | 4880563 | NM_015134.2 | NM_015134 | myosin phosphatase- | M-RIP | p116Rip; RHOIP3; |
| | | | | | Rho interacting protein | | KIAA0864 |
| | | | | | (M-RIP), transcript | | |
| | | | | | variant 1, mRNA. | | |
| 1015 | 1001 | 4880689 | NM_020243.4 | NM_020243 | translocase of outer | TOMM22 | T0M22; MSTP065; |
| | | | | | mitochondrial | | MST065; 1C9-2 |
| | | | | | membrane 22 homolog | | |
| | | | | | (yeast) (TOMM22), | | |
| | | | | | nuclear gene encoding | | |
| | | | | | mitochondrial protein, | | |
| | | | | | mRNA. | | |
| 1016 | 1002 | 4880703 | NM_053067.1 | NM_053067 | ubiquilin 1 (UBQLN1), | UBQLN1 | DA41; XDRP1; DSK2; |
| | | | | | transcript variant 2, | | FLJ90054; PLIC-1 |
| | | | | | mRNA. | | |
| 1017 | 1003 | 4890181 | NM_002885.1 | NM_002885 | RAP1 GTPase | RAP1GAP | KIAA0474; |
| | | | | | activating protein | | Rap1GAP1; |
| | | | | | (RAP1GAP), mRNA. | | RAP1GA1; rap1GAPII |
| 1018 | 1004 | 4890255 | NM_024758.3 | NM_024758 | agmatineureohydrolase | AGMAT | FLJ23384 |
| | | | | | (agmatinase) | | |
| | | | | | (AGMAT), mRNA. | | |
| 1019 | 1005 | 4900070 | NM_004832.1 | NM_004832 | glutathione S- | GSTO1 | GSTTLp28; P28; |
| | | | | | transferase omega 1 | | DKFZp686H13163 |
| | | | | | (GSTO1), mRNA. | | |
| 1020 | 1006 | 4900685 | NM_001040194.1 | NM_001040194 | angiotensin II receptor- | AGTRAP | MGC29646; ATRAP |
| | | | | | associated protein | | |
| | | | | | (AGTRAP), transcript | | |
| | | | | | variant 2, mRNA. | | |
| 1021 | 1007 | 4900707 | NR_001588.1 | NR_001588 | Shwachman-Bodian- | SBDSP | — |
| | | | | | Diamond syndrome | | |
| | | | | | pseudogene (SBDSP) | | |
| | | | | | on chromosome 7. | | |
| 1022 | 1008 | 4920403 | NM_018427.3 | NM_018427 | RRN3 RNA | RRN3 | MGC104238; TIFIA; |
| | | | | | polymerase I | | DKFZp566E104 |
| | | | | | transcription factor | | |
| | | | | | homolog (S. cerevisiae) | | |
| | | | | | (RRN3), mRNA. | | |
| 1023 | 1009 | 5050349 | NM_001018038.1 | NM_001018038 | vacuolar protein sorting | VPS13A | CHAC; FLJ42030; |
| | | | | | 13 homolog A (S. cerevisiae) | | KIAA0986 |
| | | | | | (VPS13A), | | |
| | | | | | transcript variant D, | | |
| | | | | | mRNA. | | |
| 1024 | 1010 | 5050408 | NM_005066.1 | NM_005066 | splicing factor | SFPQ | POMP100; PSF |
| | | | | | proline/glutamine-rich | | |
| | | | | | (polypyrimidine tract | | |
| | | | | | binding protein | | |
| | | | | | associated) (SFPQ), | | |
| | | | | | mRNA. | | |
| 1025 | 1011 | 5050437 | NM_016558.2 | NM_016558 | SCAN domain | SCAND1 | RAZ1; SDP1 |
| | | | | | containing 1 | | |
| | | | | | (SCAND1), transcript | | |
| | | | | | variant 1, mRNA. | | |
| 1026 | 1012 | 5050441 | NM_014795.2 | NM_014795 | zinc finger E-box | ZEB2 | KIAA0569; SMADIP1; |
| | | | | | binding homeobox 2 | | ZFHX1B; SIP1; SIP-1 |
| | | | | | (ZEB2), mRNA. | | |
| 1027 | 1013 | 5050520 | NM_022827.2 | NM_022827 | spermatogenesis | SPATA20 | MGC111032; |
| | | | | | associated 20 | | FLJ21347; Tisp78; |
| | | | | | (SPATA20), mRNA. | | DKFZp686H1839; |
| | | | | | | | SSP411; FLJ21969 |
| 1028 | 1014 | 5050603 | NM_198517.2 | NM_198517 | TBC1 domain family, | TBC1D10C | MGC46488; |
| | | | | | member 10C | | FLJ00332 |
| | | | | | (TBC1D10C), mRNA. | | |
| 1029 | 1015 | 5050634 | NM_014468.2 | NM_014468 | VENT homeobox | VENTX | HPX42B; |
| | | | | | homolog | | MGC119910; |
| | | | | | (Xenopus laevis) | | MGC119911; |
| | | | | | (VENTX), mRNA. | | VENTX2; NA88A |
| 1030 | 1016 | 5050681 | NM_017899.2 | NM_017899 | tescalcin (TESC), | TESC | TSC; FLJ20607 |
| | | | | | mRNA. | | |
| 1031 | 1017 | 5050753 | NM_001013703.2 | NM_001013703 | eukaryotic translation | EIF2AK4 | GCN2; KIAA1338 |
| | | | | | initiation factor 2 alpha | | |
| | | | | | kinase 4 (EIF2AK4), | | |
| | | | | | mRNA. | | |
| 1032 | 1018 | 5080021 | NM_001165.3 | NM_001165 | baculoviral IAP repeat- | BIRC3 | RNF49; MALT2; |
| | | | | | containing 3 (BIRC3), | | MIHC; HAIP1; API2; |
| | | | | | transcript variant 1, | | HIAP1; AIP1; CIAP2 |
| | | | | | mRNA. | | |
| 1033 | 1019 | 5080333 | NM_080686.1 | NM_080686 | HLA-B associated | BAT2 | G2; D6S51E; |
| | | | | | transcript 2 (BAT2), | | DKFZp686D09175; |
| | | | | | transcript variant 1, | | D6S51 |
| | | | | | mRNA. | | |
| 1034 | 1021 | 5090040 | NM_198219.1 | NM_198219 | inhibitor of growth | ING1 | p33ING1; p33; |
| | | | | | family, member 1 | | p47ING1a; |
| | | | | | (ING1), transcript | | p33ING1b; p47; |
| | | | | | variant 1, mRNA. | | p24ING1c |
| 1035 | 1022 | 5090068 | NM_003164.3 | NM_003164 | syntaxin 5 (STX5), | STX5 | STX5A; SED5 |
| | | | | | mRNA. | | |
| 1036 | 1023 | 5090088 | NM_004446.2 | NM_004446 | glutamyl-prolyl- | EPRS | QPRS; EARS; PARS; |
| | | | | | tRNAsynthetase | | PIG32; QARS; |
| | | | | | (EPRS), mRNA. | | DKFZp313B047 |
| 1037 | 1024 | 5090156 | NM_182744.1 | NM_182744 | neuroblastoma, | NBL1 | D1S1733E; NO3; |
| | | | | | suppression of | | DAND1; DAN; NB; |
| | | | | | tumorigenicity 1 | | MGC8972 |
| | | | | | (NBL1), transcript | | |
| | | | | | variant 1, mRNA. | | |
| 1038 | 1025 | 5090300 | NM_002562.4 | NM_002562 | purinergic receptor | P2RX7 | MGC20089; P2X7 |
| | | | | | P2X, ligand-gated ion | | |
| | | | | | channel, 7 (P2RX7), | | |
| | | | | | mRNA. | | |
| 1039 | 1026 | 5090376 | NM_006555.3 | NM_006555 | YKT6 v-SNARE | YKT6 | — |
| | | | | | homolog (S. cerevisiae) | | |
| | | | | | (YKT6), mRNA. | | |
| 1040 | 1027 | 5090400 | NM_018241.1 | NM_018241 | transmembrane protein | TMEM34 | FLJ10846 |
| | | | | | 34 (TMEM34), mRNA. | | |
| 1041 | 1028 | 5090402 | NM_016374.5 | NM_016374 | AT rich interactive | ARID4B | BCAA; RBP1L1; |
| | | | | | domain 4B (RBP1-like) | | MGC163290; |
| | | | | | (ARID4B), transcript | | BRCAA1; SAP180; |
| | | | | | variant 1, mRNA. | | RBBP1L1; |
| | | | | | | | DKFZp313M2420 |
| 1042 | 1029 | 5130114 | NM_021871.2 | NM_021871 | fibrinogen alpha chain | FGA | MGC119425; |
| | | | | | (FGA), transcript variant | | MGC119423; Fib2; |
| | | | | | alpha, mRNA. | | MGC119422 |
| 1043 | 1030 | 5130139 | NM_001537.2 | NM_001537 | heat shock factor | HSBP1 | DKFZp686D1664; |
| | | | | | binding protein 1 | | NPC-A-13; |
| | | | | | (HSBP1), mRNA. | | DKFZp686O24200 |
| 1044 | 1031 | 5130440 | NM_139346.1 | NM_139346 | bridging integrator 1 | BIN1 | MGC10367; AMPH2; |
| | | | | | (BIN1), transcript | | DKFZp547F068; |
| | | | | | variant 4, mRNA. | | SH3P9; AMPHL |
| 1045 | 1032 | 5130605 | NM_012123.2 | NM_012123 | mitochondrial | MTO1 | CGI-02 |
| | | | | | translation optimization | | |
| | | | | | 1 homolog (S. cerevisiae) | | |
| | | | | | (MTO1), | | |
| | | | | | nuclear gene encoding | | |
| | | | | | mitochondrial protein, | | |
| | | | | | transcript variant 2, | | |
| | | | | | mRNA. | | |
| 1046 | 1033 | 5220161 | NM_001001.3 | NM_001001 | ribosomal protein L36a- | RPL36AL | RPL36A; |
| | | | | | like (RPL36AL), mRNA. | | MGC111574 |
| 1047 | 1034 | 5220497 | NM_001037171.1 | NM_001037171 | acyl-CoA thioesterase 9 | ACOT9 | MT-ACT48; CGI-16; |
| | | | | | (ACOT9), transcript | | ACATE2 |
| | | | | | variant 1, mRNA. | | |
| 1048 | 1035 | 5220504 | NM_001011671.1 | NM_001011671 | coiled-coil-helix-coiled- | CHCHD7 | FLJ40966; MGC2217 |
| | | | | | coil-helix domain | | |
| | | | | | containing 7 | | |
| | | | | | (CHCHD7), transcript | | |
| | | | | | variant 6, mRNA. | | |
| 1049 | 1036 | 5220653 | NM_004539.3 | NM_004539 | asparaginyl- | NARS | ASNRS; NARS1 |
| | | | | | tRNAsynthetase | | |
| | | | | | (NARS), mRNA. | | |
| 1050 | 1037 | 5260082 | NM_014711.3 | NM_014711 | CP110 protein | CP110 | KIAA0419; |
| | | | | | (CP110), mRNA. | | DKFZp781G1416 |
| 1051 | 1038 | 5260369 | NM_145290.2 | NM_145290 | G protein-coupled | GPR125 | TEM5L; PGR21 |
| | | | | | receptor 125 | | |
| | | | | | (GPR125), mRNA. | | |
| 1052 | 1039 | 5260575 | NM_003677.3 | NM_003677 | density-regulated | DENR | DRP; DRP1; SMAP-3 |
| | | | | | protein (DENR), | | |
| | | | | | mRNA. | | |
| 1053 | 1040 | 5270575 | NM_001040439.1 | NM_001040439 | mitogen-activated | MAPK8IP3 | SYD2; KIAA1066; |
| | | | | | protein kinase 8 | | FLJ00027; JSAP1; |
| | | | | | interacting protein 3 | | DKFZp762N1113; |
| | | | | | (MAPK8IP3), transcript | | JIP3 |
| | | | | | variant 2, mRNA. | | |
| 1054 | 1041 | 5270768 | NM_031268.4 | NM_031268 | 3-phosphoinositide | PDPK1 | MGC20087; |
| | | | | | dependent protein | | MGC35290; PDK1; |
| | | | | | kinase-1 (PDPK1), | | PRO0461; PkB-like; |
| | | | | | transcript variant 2, | | PkB-like 1 |
| | | | | | mRNA. | | |
| 1055 | 1043 | 5290131 | NM_006141.2 | NM_006141 | dynein, cytoplasmic 1, | DYNC1LI2 | DNCLI2; LIC2 |
| | | | | | light intermediate chain | | |
| | | | | | 2 (DYNC1LI2), mRNA. | | |
| 1056 | 1044 | 5290193 | NM_153331.2 | NM_153331 | potassium channel | KCTD6 | MGC27385 |
| | | | | | tetramerisation domain | | |
| | | | | | containing 6 (KCTD6), | | |
| | | | | | mRNA. | | |
| 1057 | 1045 | 5290397 | NM_013246.2 | NM_013246 | cardiotrophin-like | CLCF1 | CISS2; NNT1; CLC; |
| | | | | | cytokine factor 1 | | BSF3; NR6 |
| | | | | | (CLCF1), mRNA. | | |
| 1058 | 1046 | 5290576 | NR_003264.1 | NR_003264 | succinate | SDHALP1 | SDHAL1 |
| | | | | | dehydrogenase | | |
| | | | | | complex, subunit A, | | |
| | | | | | flavoproteinpseudogene | | |
| | | | | | 1 (SDHALP1) on | | |
| | | | | | chromosome 3. | | |
| 1059 | 1047 | 5290692 | NM_005827.1 | NM_005827 | solute carrier family 35, | SLC35B1 | UGTREL1 |
| | | | | | member B1 | | |
| | | | | | (SLC35B1), mRNA. | | |
| 1060 | 1048 | 5310170 | NM_001018070.1 | NM_001018070 | coronin, actin binding | CORO1B | CORONIN-2; |
| | | | | | protein, 1B (CORO1B), | | DKFZP762I166 |
| | | | | | transcript variant 2, | | |
| | | | | | mRNA. | | |
| 1061 | 1049 | 5310397 | NM_145256.2 | NM_145256 | leucine rich repeat | LRRC25 | MAPA; FLJ38116 |
| | | | | | containing 25 | | |
| | | | | | (LRRC25), mRNA. | | |
| 1062 | 1050 | 5360064 | NM_012483.1 | NM_012483 | granulysin (GNLY), | GNLY | D2S69E; 519; LAG2; |
| | | | | | transcript variant 519, | | NKG5; LAG-2; |
| | | | | | mRNA. | | TLA519 |
| 1063 | 1051 | 5360154 | NM_017740.1 | NM_017740 | zinc finger, DHHC-type | ZDHHC7 | ZNF370; FLJ10792; |
| | | | | | containing 7 | | FLJ20279 |
| | | | | | (ZDHHC7), mRNA. | | |
| 1064 | 1052 | 5360653 | NM_000098.1 | NM_000098 | carnitinepalmitoyltransferase | CPT2 | CPTASE; CPT1 |
| | | | | | II (CPT2), | | |
| | | | | | nuclear gene encoding | | |
| | | | | | mitochondrial protein, | | |
| | | | | | mRNA. | | |
| 1065 | 1053 | 5360703 | NM_016453.2 | NM_016453 | NCK interacting protein | NCKIPSD | DIP; WISH; AF3P21; |
| | | | | | with SH3 domain | | MGC23891; SPIN90; |
| | | | | | (NCKIPSD), transcript | | DIP1; ORF1; |
| | | | | | variant 1, mRNA. | | WASLBP |
| 1066 | 1054 | 5360707 | NM_001044387.1 | NM_001044387 | zinc finger protein 557 | ZNF557 | MGC4054 |
| | | | | | (ZNF557), transcript | | |
| | | | | | variant 2, mRNA. | | |
| 1067 | 1055 | 5390010 | NM_015266.1 | NM_015266 | solute carrier family 9 | SLC9A8 | FLJ42500; |
| | | | | | (sodium/hydrogen | | MGC138418; NHE8; |
| | | | | | exchanger), member 8 | | DKFZp686C03237; |
| | | | | | (SLC9A8), mRNA. | | KIAA0939 |
| 1068 | 1056 | 5390128 | NM_018176.2 | NM_018176 | leucine-rich repeat LGI | LGI2 | KIAA1916; |
| | | | | | family, member 2 | | MGC126808; |
| | | | | | (LGI2), mRNA. | | MGC126810; |
| | | | | | | | FLJ10675; LGIL2 |
| 1069 | 1057 | 5390161 | NM_004419.3 | NM_004419 | dual specificity | DUSP5 | HVH3; DUSP |
| | | | | | phosphatase 5 | | |
| | | | | | (DUSP5), mRNA. | | |
| 1070 | 1058 | 5390349 | NM_001012452.1 | NM_001012452 | golgin-like hypothetical | FLJ32679 | MGC102859; |
| | | | | | protein LOC440321 | | MGC104696 |
| | | | | | (FLJ32679), mRNA. | | |
| 1071 | 1059 | 5390494 | NM_001417.4 | NM_001417 | eukaryotic translation | EIF4B | EIF-4B; PRO1843 |
| | | | | | initiation factor 4B | | |
| | | | | | (EIF4B), mRNA. | | |
| 1072 | 1060 | 5390703 | NM_153334.3 | NM_153334 | scavenger receptor | SCARF2 | SREC2; SRECRP-1; |
| | | | | | class F, member 2 | | SREC-II; NSR1 |
| | | | | | (SCARF2), transcript | | |
| | | | | | variant 1, mRNA. | | |
| 1073 | 1061 | 5420367 | NM_000454.4 | NM_000454 | superoxide dismutase | SOD1 | ALS1; IPOA; SOD; |
| | | | | | 1, soluble (amyotrophic | | homodimer, ALS |
| | | | | | lateral sclerosis 1 | | |
| | | | | | (adult)) (SOD1), | | |
| | | | | | mRNA. | | |
| 1074 | 1062 | 5420377 | NM_002306.1 | NM_002306 | lectin, galactoside- | LGALS3 | GAL3; MAC2; |
| | | | | | binding, soluble, 3 | | LGALS2; CBP35; |
| | | | | | (galectin 3) (LGALS3), | | GALBP |
| | | | | | mRNA. | | |
| 1075 | 1063 | 5420564 | NM_005384.2 | NM_005384 | nuclear factor, | NFIL3 | IL3BP1; NF-IL3A; |
| | | | | | interleukin 3 regulated | | E4BP4; NFIL3A |
| | | | | | (NFIL3), mRNA. | | |
| 1076 | 1064 | 5490367 | NM_175868.1 | NM_175868 | melanoma antigen | MAGEA6 | MAGE3B; MAGE-3b; |
| | | | | | family A, 6 (MAGEA6), | | MGC52297; MAGE6 |
| | | | | | transcript variant 2, | | |
| | | | | | mRNA. | | |
| 1077 | 1065 | 5490402 | NM_016202.2 | NM_016202 | zinc finger protein 580 | ZNF580 | — |
| | | | | | (ZNF580), transcript | | |
| | | | | | variant 1, mRNA. | | |
| 1078 | 1066 | 5560075 | NM_005928.1 | NM_005928 | milk fat globule-EGF | MFGE8 | HsT19888; BA46; |
| | | | | | factor 8 protein | | OAcGD3S; EDIL1 |
| | | | | | (MFGE8), mRNA. | | |
| 1079 | 1068 | 5560136 | NM_018117.10 | NM_0181170 | bromodomain and WD | BRWD2 | FLJ42531; DR11; |
| | | | | | repeat domain | | DKFZp434L1715; |
| | | | | | containing 2 (BRWD2), | | WDR15; WDR11 |
| | | | | | mRNA. | | |
| 1080 | 1069 | 5560471 | NM_133280.1 | NM_133280 | Fc fragment of IgA, | FCAR | CD89 |
| | | | | | receptor for (FCAR), | | |
| | | | | | transcript variant 10, | | |
| | | | | | mRNA. | | |
| 1081 | 1070 | 5560541 | NM_006929.4 | NM_006929 | superkillerviralicidic | SKIV2L | SKIV2; DDX13; |
| | | | | | activity 2-like (S. cerevisiae) | | SKI2W; SKI2; 170A; |
| | | | | | (SKIV2L), | | HLP |
| | | | | | mRNA. | | |
| 1082 | 1071 | 5560561 | NM_022748.10 | NM_0227480 | tensin 3 (TNS3), | TNS3 | TENS1; FLJ13732; |
| | | | | | mRNA. | | TEM6; MGC88434; |
| | | | | | | | H_NH049I23.2; |
| | | | | | | | FLJ35545; |
| | | | | | | | DKFZp686M1045; |
| | | | | | | | DKFZp686K12123 |
| 1083 | 1072 | 5560682 | NM_000421.2 | NM_000421 | keratin 10 | KRT10 | K10; KPP; CK10 |
| | | | | | (epidermolytic | | |
| | | | | | hyperkeratosis; | | |
| | | | | | keratosis palmaris et | | |
| | | | | | plantaris) (KRT10), | | |
| | | | | | mRNA. | | |
| 1084 | 1073 | 5570070 | NM_012400.2 | NM_012400 | phospholipase A2, | PLA2G2D | SPLASH; sPLA2S |
| | | | | | group IID (PLA2G2D), | | |
| | | | | | mRNA. | | |
| 1085 | 1074 | 5570139 | NM_012413.3 | NM_012413 | glutaminyl-peptide | QPCT | QC; GCT |
| | | | | | cyclotransferase | | |
| | | | | | (glutaminylcyclase) | | |
| | | | | | (QPCT), mRNA. | | |
| 1086 | 1075 | 5570195 | NM_033656.2 | NM_033656 | bromodomain and WD | BRWD1 | C21orf107; |
| | | | | | repeat domain | | FLJ43918; WDR9; |
| | | | | | containing 1 (BRWD1), | | N143 |
| | | | | | transcript variant 2, | | |
| | | | | | mRNA. | | |
| 1087 | 1076 | 5570242 | NM_002626.4 | NM_002626 | phosphofructokinase, | PFKL | FLJ40909; |
| | | | | | liver (PFKL), transcript | | DKFZp686G1648; |
| | | | | | variant 2, mRNA. | | PFK-B; FLJ30173; |
| | | | | | | | DKFZp686L2097 |
| 1088 | 1077 | 5570309 | NM_005131.2 | NM_005131 | THO complex 1 | THOC1 | P84; P84N5; HPR1 |
| | | | | | (THOC1), mRNA. | | |
| 1089 | 1079 | 5670315 | NM_016199.1 | NM_016199 | LSM7 homolog, U6 | LSM7 | YNL147W |
| | | | | | small nuclear RNA | | |
| | | | | | associated (S. cerevisiae) | | |
| | | | | | (LSM7), | | |
| | | | | | mRNA. | | |
| 1090 | 1080 | 5670719 | NM_000628.3 | NM_000628 | interleukin 10 receptor, | IL10RB | IL-10R2; CRF2-4; |
| | | | | | beta (IL10RB), mRNA. | | CRFB4; CDW210B; |
| | | | | | | | D21S58; D21S66 |
| 1091 | 1081 | 5690082 | NM_153373.1 | NM_153373 | alanine-glyoxylate | AGXT2L2 | MGC117348; |
| | | | | | aminotransferase 2-like | | MGC15875; |
| | | | | | 2 (AGXT2L2), mRNA. | | MGC45484 |
| 1092 | 1082 | 5690156 | NM_022492.3 | NM_022492 | tetratricopeptide repeat | TTC31 | FLJ33201; FLJ12788; |
| | | | | | domain 31 (TTC31), | | MGC120200 |
| | | | | | mRNA. | | |
| 1093 | 1083 | 5690279 | NM_006567.3 | NM_006567 | phenylalanyl- | FARS2 | dJ520B18.2; FARS1; |
| | | | | | tRNAsynthetase 2, | | HSPC320; PheRS |
| | | | | | mitochondrial (FARS2), | | |
| | | | | | nuclear gene encoding | | |
| | | | | | mitochondrial protein, | | |
| | | | | | mRNA. | | |
| 1094 | 1086 | 5690382 | NM_005605.3 | NM_005605 | protein phosphatase 3 | PPP3CC | CALNA3 |
| | | | | | (formerly 2B), catalytic | | |
| | | | | | subunit, gamma | | |
| | | | | | isoform (PPP3CC), | | |
| | | | | | mRNA. | | |
| 1095 | 1087 | 5690400 | NR_003082.1 | NR_003082 | glutathione S- | GSTTP2 | FLJ46109 |
| | | | | | transferase theta | | |
| | | | | | pseudogene 2 | | |
| | | | | | (GSTTP2) on | | |
| | | | | | chromosome 22. | | |
| | | | | | XM_941198 | | |
| | | | | | XM_945014 | | |
| | | | | | XM_945016 | | |
| 1096 | 1088 | 5690437 | NM_004515.2 | NM_004515 | interleukin enhancer | ILF2 | MGC8391; PRO3063; |
| | | | | | binding factor 2, 45 kDa | | NF45 |
| | | | | | (ILF2), mRNA. | | |
| 1097 | 1089 | 5700020 | NM_014155.2 | NM_014155 | BTB (POZ) domain | BTBD15 | MGC57431; |
| | | | | | containing 15 | | MGC60348; |
| | | | | | (BTBD15), mRNA. | | MGC88058; |
| | | | | | | | HSPC063; |
| | | | | | | | MGC26123 |
| 1098 | 1090 | 5700142 | NM_004462.3 | NM_004462 | farnesyl- | FDFT1 | SS; DGPT; SQS; |
| | | | | | diphosphatefarnesyltransferase | | ERG9 |
| | | | | | 1 (FDFT1), | | |
| | | | | | mRNA. | | |
| 1099 | 1091 | 5700220 | NM_004938.2 | NM_004938 | death-associated | DAPK1 | DAPK; |
| | | | | | protein kinase 1 | | DKFZp781I035 |
| | | | | | (DAPK1), mRNA. | | |
| 1100 | 1092 | 5700309 | NM_199336.1 | NM_199336 | fumarylacetoacetate | FAHD2B | DKFZp434N062 |
| | | | | | hydrolase domain | | |
| | | | | | containing 2B | | |
| | | | | | (FAHD2B), mRNA. | | |
| 1101 | 1093 | 5700672 | NM_006464.2 | NM_006464 | trans-golgi network | TGOLN2 | TGN51; MGC14722; |
| | | | | | protein 2 (TGOLN2), | | TGN48; TGN38; |
| | | | | | mRNA. | | TGN46; TTGN2 |
| 1102 | 1094 | 5700722 | NM_003310.1 | NM_003310 | tumor suppressing | TSSC1 | — |
| | | | | | subtransferable | | |
| | | | | | candidate 1 (TSSC1), | | |
| | | | | | mRNA. | | |
| 1103 | 1095 | 5720056 | NM_032286.2 | NM_032286 | mediator complex | MED10 | L6; MGC5309; NUT2; |
| | | | | | subunit 10 (MED10), | | TRG20 |
| | | | | | mRNA. | | |
| 1104 | 1096 | 5720180 | NM_001466.2 | NM_001466 | frizzled homolog 2 | FZD2 | — |
| | | | | | (Drosophila) (FZD2), | | |
| | | | | | mRNA. | | |
| 1105 | 1097 | 5720273 | NM_000999.2 | NM_000999 | ribosomal protein L38 | RPL38 | — |
| | | | | | (RPL38), mRNA. | | |
| 1106 | 1098 | 5720373 | NM_001012302.2 | NM_001012302 | transmembrane protein | TMEM16J | TP53I5; PIG5 |
| | | | | | 16J (TMEM16J), | | |
| | | | | | mRNA. | | |
| 1107 | 1099 | 5720398 | NM_174889.3 | NM_174889 | NADH dehydrogenase | NDUFAF2 | FLJ22398; mimitin; |
| | | | | | (ubiquinone) 1 alpha | | B17.2L; MMTN; |
| | | | | | subcomplex, assembly | | NDUFA12L |
| | | | | | factor 2 (NDUFAF2), | | |
| | | | | | mRNA. | | |
| 1108 | 1100 | 5810142 | NM_133467.2 | NM_133467 | Cbp/p300-interacting | CITED4 | — |
| | | | | | transactivator, with | | |
| | | | | | Glu/Asp-rich carboxy- | | |
| | | | | | terminal domain, 4 | | |
| | | | | | (CITED4), mRNA. | | |
| 1109 | 1101 | 5810367 | NM_018177.2 | NM_018177 | Nedd4 binding protein 2 | N4BP2 | B3BP; KIAA1413; |
| | | | | | (N4BP2), mRNA. | | FLJ10680 |
| 1110 | 1102 | 5810605 | NM_006835.2 | NM_006835 | cyclin I (CCNI), mRNA. | CCNI | CYC1; CYI |
| 1111 | 1103 | 5810612 | NM_001150.1 | NM_001150 | alanyl (membrane) | ANPEP | APN; gp150; CD13; |
| | | | | | aminopeptidase | | LAP1; PEPN |
| | | | | | (aminopeptidase N, | | |
| | | | | | aminopeptidase M, | | |
| | | | | | microsomal | | |
| | | | | | aminopeptidase, CD13, | | |
| | | | | | p150) (ANPEP), | | |
| | | | | | mRNA. | | |
| 1112 | 1104 | 5820242 | NM_152911.2 | NM_152911 | polyamine oxidase | PAOX | PAO; RP11- |
| | | | | | (exo-N4-amino) | | 122K13.11; |
| | | | | | (PAOX), transcript | | MGC45464; |
| | | | | | variant 1, mRNA. | | DKFZp434J245 |
| 1113 | 1105 | 5820598 | NM_005481.2 | NM_005481 | mediator complex | MED16 | THRAP5; TRAP95; |
| | | | | | subunit 16 (MED16), | | DRIP92; MED16 |
| | | | | | mRNA. | | |
| 1114 | 1106 | 5860215 | NM_016006.3 | NM_016006 | abhydrolase domain | ABHD5 | MGC8731; CGI58; |
| | | | | | containing 5 (ABHD5), | | CDS; NCIE2; IECN2 |
| | | | | | mRNA. | | |
| 1115 | 1107 | 5860253 | NM_017651.3 | NM_017651 | Abelson helper | AHI1 | FLJ14023; FLJ20069; |
| | | | | | integration site 1 | | DKFZp686J1653; |
| | | | | | (AHI1), mRNA. | | JBTS3; dJ71N10.1; |
| | | | | | | | ORF1; AHI-1 |
| 1116 | 1108 | 5870164 | NM_001045557.1 | NM_001045557 | Src-like-adaptor (SLA), | SLA | SLA1; SLAP |
| | | | | | transcript variant 2, | | |
| | | | | | mRNA. | | |
| 1117 | 1109 | 5870189 | NM_152542.2 | NM_152542 | protein phosphatase 1K | PPM1K | UG0882E07; PP2Cm; |
| | | | | | (PP2C domain | | DKFZp667B084; |
| | | | | | containing) (PPM1K), | | PTMP; |
| | | | | | mRNA. | | DKFZp761G058 |
| 1118 | 1110 | 5870192 | NM_001075099.1 | NM_001075099 | src kinase associated | SKAP1 | SCAP1; SKAP55 |
| | | | | | phosphoprotein 1 | | |
| | | | | | (SKAP1), transcript | | |
| | | | | | variant 2, mRNA. | | |
| 1119 | 1111 | 5870326 | NM_014580.3 | NM_014580 | solute carrier family 2, | SLC2A8 | GLUTX1; GLUT8 |
| | | | | | (facilitated glucose | | |
| | | | | | transporter) member 8 | | |
| | | | | | (SLC2A8), mRNA. | | |
| 1120 | 1112 | 5890349 | NM_017454.2 | NM_017454 | staufen, RNA binding | STAU1 | FLJ25010; STAU |
| | | | | | protein, homolog 1 | | |
| | | | | | (Drosophila) (STAU1), | | |
| | | | | | transcript variant T1, | | |
| | | | | | mRNA. | | |
| 1121 | 1113 | 5890414 | NM_001465.3 | NM_001465 | FYN binding protein | FYB | PRO0823; SLAP-130; |
| | | | | | (FYB-120/130) (FYB), | | ADAP |
| | | | | | transcript variant 1, | | |
| | | | | | mRNA. | | |
| 1122 | 1114 | 5890497 | NM_001077446.1 | NM_001077446 | tRNA splicing | TSEN34 | LENG5; SEN34; |
| | | | | | endonuclease 34 | | SEN34L |
| | | | | | homolog (S. cerevisiae) | | |
| | | | | | (TSEN34), transcript | | |
| | | | | | variant 2, mRNA. | | |
| 1123 | 1115 | 5890554 | NM_207406.2 | NM_207406 | coiled-coil domain | CCDC4 | MGC157807; |
| | | | | | containing 4 (CCDC4), | | FLJ35632; |
| | | | | | mRNA. | | MGC157808; |
| | | | | | | | FLJ43965 |
| 1124 | 1116 | 5890564 | NM_182703.3 | NM_182703 | ankyrin repeat and | ANKDD1A | MGC120306; |
| | | | | | death domain | | FLJ25870; |
| | | | | | containing 1A | | MGC120307; |
| | | | | | (ANKDD1A), mRNA. | | MGC120305 |
| 1125 | 1117 | 5890653 | NM_000076.1 | NM_000076 | cyclin-dependent | CDKN1C | BWCR; p57; BWS; |
| | | | | | kinase inhibitor 1C | | KIP2; WBS |
| | | | | | (p57, Kip2) (CDKN1C), | | |
| | | | | | mRNA. | | |
| 1126 | 1118 | 5890730 | XR_017804.1 | XR_017804 | PREDICTED: 40S | RPS26L | — |
| | | | | | ribosomal protein S26- | | |
| | | | | | like (RPS26L), misc | | |
| | | | | | RNA. | | |
| 1127 | 1119 | 5900379 | NM_003134.2 | NM_003134 | signal recognition | SRP14 | ALURBP; MGC14326 |
| | | | | | particle 14 kDa | | |
| | | | | | (homologous Alu RNA | | |
| | | | | | binding protein) | | |
| | | | | | (SRP14), mRNA. | | |
| 1128 | 1120 | 5900468 | NM_006037.3 | NM_006037 | histone deacetylase 4 | HDAC4 | HD4; HDAC-A; |
| | | | | | (HDAC4), mRNA. | | KIAA0288; HA6116; |
| | | | | | | | HDACA |
| 1129 | 1121 | 5900592 | NM_002406.2 | NM_002406 | mannosyl (alpha-1,3-)- | MGAT1 | GLYT1; GLCT1; |
| | | | | | glycoprotein beta-1,2- | | GNT-1; GLCNAC-TI; |
| | | | | | N- | | MGAT; GNT-I |
| | | | | | acetylglucosaminyltransferase | | |
| | | | | | (MGAT1), | | |
| | | | | | mRNA. | | |
| 1130 | 1122 | 5900692 | NM_015917.1 | NM_015917 | glutathione S- | GSTK1 | GST13 |
| | | | | | transferase kappa 1 | | |
| | | | | | (GSTK1), mRNA. | | |
| 1131 | 1123 | 5900725 | NM_007350.3 | NM_007350 | pleckstrin homology- | PHLDA1 | DT1P1B11; PHRIP; |
| | | | | | like domain, family A, | | TDAG51; |
| | | | | | member 1 (PHLDA1), | | MGC131738 |
| | | | | | mRNA. | | |
| 1132 | 1124 | 5900739 | NM_152230.3 | NM_152230 | inositol polyphosphate | IPMK | — |
| | | | | | multikinase (IPMK), | | |
| | | | | | mRNA. | | |
| 1133 | 1125 | 5910112 | NM_001012636.1 | NM_001012636 | interleukin 32 (IL32), | IL32 | TAIFb; IL-32alpha; |
| | | | | | transcript variant 7, | | TAIFa; TAIFd; IL- |
| | | | | | mRNA. | | 32beta; IL-32delta; |
| | | | | | | | TAIFc; TAIF; NK4; IL- |
| | | | | | | | 32gamma |
| 1134 | 1126 | 5910523 | NM_006769.2 | NM_006769 | LIM domain only 4 | LMO4 | — |
| | | | | | (LMO4), mRNA. | | |
| 1135 | 1127 | 5910632 | NM_003078.3 | NM_003078 | SWI/SNF related, | SMARCD3 | Rsc6p; BAF60C; |
| | | | | | matrix associated, actin | | CRACD3; |
| | | | | | dependent regulator of | | MGC111010 |
| | | | | | chromatin, subfamily d, | | |
| | | | | | member 3 | | |
| | | | | | (SMARCD3), transcript | | |
| | | | | | variant 2, mRNA. | | |
| 1136 | 1128 | 5960097 | NM_001126.2 | NM_001126 | adenylosuccinate | ADSS | ADEH; MGC20404 |
| | | | | | synthase (ADSS), | | |
| | | | | | mRNA. | | |
| 1137 | 1129 | 5960343 | NM_033405.2 | NM_033405 | peroxisomal | PRIC285 | MGC138228; PDIP-1; |
| | | | | | proliferator-activated | | PDIP1alpha; |
| | | | | | receptor A interacting | | KIAA1769; |
| | | | | | complex 285 | | PDIP1beta; |
| | | | | | (PRIC285), transcript | | MGC132634; |
| | | | | | variant 2, mRNA. | | FLJ00244 |
| 1138 | 1130 | 5960520 | NM_021819.2 | NM_021819 | lectin, mannose- | LMAN1L | ERGL; ERGIC-53L |
| | | | | | binding, 1 like | | |
| | | | | | (LMAN1L), mRNA. | | |
| 1139 | 1131 | 6020093 | NM_006513.2 | NM_006513 | seryl-tRNAsynthetase | SARS | SERRS; FLJ36399; |
| | | | | | (SARS), mRNA. | | SERS |
| 1140 | 1132 | 6020474 | NM_018445.4 | NM_018445 | selenoprotein S | SELS | SEPS1; ADO15; |
| | | | | | (SELS), transcript | | MGC2553; SBBI8; |
| | | | | | variant 2, mRNA. | | VIMP; AD-015; |
| | | | | | | | MGC104346 |
| 1141 | 1133 | 6020491 | NM_198220.1 | NM_198220 | small nuclear | SNRPB2 | MGC45309; |
| | | | | | ribonucleoprotein | | MGC24807 |
| | | | | | polypeptide B″ | | |
| | | | | | (SNRPB2), transcript | | |
| | | | | | variant 2, mRNA. | | |
| 1142 | 1134 | 6040064 | NM_001006115.2 | NM_001006115 | inositol hexaphosphate | IHPK1 | PiUS; IP6K1; |
| | | | | | kinase 1 (IHPK1), | | MGC9925 |
| | | | | | transcript variant 2, | | |
| | | | | | mRNA. | | |
| 1143 | 1135 | 6040670 | NR_001561.1 | NR_001561 | cytochrome c, somatic- | CYCSL1 | HS11; bA513I15.3; |
| | | | | | like 1 (CYCSL1) on | | HCP15 |
| | | | | | chromosome 6. | | |
| 1144 | 1136 | 6060148 | NM_014563.3 | NM_014563 | trafficking protein | TRAPPC2 | hYP38334; TRS20; |
| | | | | | particle complex 2 | | SEDT; SEDL; |
| | | | | | (TRAPPC2), transcript | | ZNF547L; MIP-2A |
| | | | | | variant 2, mRNA. | | |
| 1145 | 1137 | 6060201 | NM_006773.3 | NM_006773 | DEAD (Asp-Glu-Ala- | DDX18 | FLJ33908; MrDb |
| | | | | | Asp) box polypeptide | | |
| | | | | | 18 (DDX18), mRNA. | | |
| 1146 | 1138 | 6060377 | NM_016818.2 | NM_016818 | ATP-binding cassette, | ABCG1 | WHITE1; ABC8; |
| | | | | | sub-family G (WHITE), | | MGC34313 |
| | | | | | member 1 (ABCG1), | | |
| | | | | | transcript variant 2, | | |
| | | | | | mRNA. | | |
| 1147 | 1139 | 6060433 | NM_001006944.1 | NM_001006944 | ribosomal protein S6 | RPS6KA4 | RSK-B; MSK2 |
| | | | | | kinase, 90 kDa, | | |
| | | | | | polypeptide 4 | | |
| | | | | | (RPS6KA4), transcript | | |
| | | | | | variant 2, mRNA. | | |
| 1148 | 1140 | 6060468 | NM_002964.3 | NM_002964 | S100 calcium binding | S100A8 | CFAG; 60B8AG; |
| | | | | | protein A8 (S100A8), | | MRP8; CGLA; L1Ag; |
| | | | | | mRNA. | | MIF; MA387; CAGA; |
| | | | | | | | CP-10; NIF; P8 |
| 1149 | 1141 | 6060475 | NM_001204.5 | NM_001204 | bone morphogenetic | BMPR2 | BRK-3; BMPR3; |
| | | | | | protein receptor, type II | | BMPR-II; BMR2; |
| | | | | | (serine/threonine | | TRG10; PPH1; T-ALK |
| | | | | | kinase) (BMPR2), | | |
| | | | | | mRNA. | | |
| 1150 | 1142 | 6060685 | NM_153485.1 | NM_153485 | nucleoporin 155 kDa | NUP155 | N155; KIAA0791 |
| | | | | | (NUP155), transcript | | |
| | | | | | variant 1, mRNA. | | |
| 1151 | 1143 | 6060731 | NM_012325.1 | NM_012325 | microtubule-associated | MAPRE1 | EB1; MGC129946; |
| | | | | | protein, RP/EB family, | | MGC117374 |
| | | | | | member 1 (MAPRE1), | | |
| | | | | | mRNA. | | |
| 1152 | 1144 | 6100022 | NM_003517.2 | NM_003517 | histone cluster 2, H2ac | HIST2H2AC | H2AFQ; MGC74460; |
| | | | | | (HIST2H2AC), mRNA. | | H2A; H2A/q; H2A- |
| | | | | | | | GL101 |
| 1153 | 1145 | 6100364 | NM_022893.3 | NM_022893 | B-cell CLL/lymphoma | BCL11A | BCL11A-L; CTIP1; |
| | | | | | 11A (zinc finger protein) | | FLJ10173; EVI9; |
| | | | | | (BCL11A), transcript | | BCL11A-XL; |
| | | | | | variant 1, mRNA. | | HBFQTL5; BCL11A- |
| | | | | | | | S; FLJ34997; |
| | | | | | | | KIAA1809 |
| 1154 | 1146 | 6100482 | NM_001001396.1 | NM_001001396 | ATPase, Ca++ | ATP2B4 | DKFZp686M088; |
| | | | | | transporting, plasma | | PMCA4b; |
| | | | | | membrane 4 | | DKFZp686G08106; |
| | | | | | (ATP2B4), transcript | | PMCA4x; PMCA4; |
| | | | | | variant 1, mRNA. | | ATP2B2; MXRA1 |
| 1155 | 1147 | 6100630 | NM_032853.2 | NM_032853 | melanoma associated | MUM1 | MGC163315; |
| | | | | | antigen (mutated) 1 | | MGC131891; |
| | | | | | (MUM1), mRNA. | | HSPC211; FLJ22283; |
| | | | | | | | FLJ14868; MUM-1 |
| 1156 | 1148 | 6100703 | NM_002880.2 | NM_002880 | v-raf-1 murine leukemia | RAF1 | Raf-1; c-Raf; NS5; |
| | | | | | viral oncogene | | CRAF |
| | | | | | homolog 1 (RAF1), | | |
| | | | | | mRNA. | | |
| 1157 | 1149 | 6100768 | NM_014376.2 | NM_014376 | cytoplasmic FMR1 | CYFIP2 | PIR121 |
| | | | | | interacting protein 2 | | |
| | | | | | (CYFIP2), transcript | | |
| | | | | | variant 3, mRNA. | | |
| 1158 | 1150 | 6110037 | NM_006865.2 | NM_006865 | leukocyte | LILRA3 | HM31; LIR-4; e3; |
| | | | | | immunoglobulin-like | | CD85E; ILT6; HM43; |
| | | | | | receptor, subfamily A | | LIR4 |
| | | | | | (without TM domain), | | |
| | | | | | member 3 (LILRA3), | | |
| | | | | | mRNA. | | |
| 1159 | 1151 | 6110731 | NM_014216.3 | NM_014216 | inositol 1,3,4- | ITPK1 | ITRPK1 |
| | | | | | triphosphate 5/6 kinase | | |
| | | | | | (ITPK1), mRNA. | | |
| 1160 | 1152 | 6110736 | NM_003749.2 | NM_003749 | insulin receptor | IRS2 | — |
| | | | | | substrate 2 (IRS2), | | |
| | | | | | mRNA. | | |
| 1161 | 1153 | 6130019 | NM_001018677.1 | NM_001018677 | farnesyltransferase, | FNTA | FPTA; MGC99680; |
| | | | | | CAAX box, alpha | | PGGT1A |
| | | | | | (FNTA), transcript | | |
| | | | | | variant 3, mRNA. | | |
| 1162 | 1154 | 6130020 | NM_003666.2 | NM_003666 | basic leucine zipper | BLZF1 | JEM1; JEM-1; |
| | | | | | nuclear factor 1 (JEM- | | GOLGIN-45; |
| | | | | | 1) (BLZF1), mRNA. | | MGC22497; JEM-1s |
| 1163 | 1155 | 6130044 | NM_032765.2 | NM_032765 | tripartite motif- | TRIM52 | RNF102; MGC16175 |
| | | | | | containing 52 | | |
| | | | | | (TRIM52), mRNA. | | |
| 1164 | 1156 | 6130326 | NM_006113.4 | NM_006113 | vav 3 guanine | VAV3 | FLJ40431 |
| | | | | | nucleotide exchange | | |
| | | | | | factor (VAV3), transcript | | |
| | | | | | variant 1, mRNA. | | |
| 1165 | 1157 | 6130438 | NM_001031720.2 | NM_001031720 | glutathione S- | GSTCD | DKFZp686I10174; |
| | | | | | transferase, C-terminal | | FLJ13273 |
| | | | | | domain containing | | |
| | | | | | (GSTCD), transcript | | |
| | | | | | variant 1, mRNA. | | |
| 1166 | 1158 | 6180019 | NM_004763.3 | NM_004763 | integrin beta 1 binding | ITGB1BP1 | ICAP1B; ICAP- |
| | | | | | protein 1 (ITGB1BP1), | | 1alpha; ICAP1A; |
| | | | | | transcript variant 1, | | ICAP1; ICAP-1B; |
| | | | | | mRNA. | | DKFZp686K08158; |
| | | | | | | | ICAP-1A |
| 1167 | 1159 | 6180039 | NM_001005.3 | NM_001005 | ribosomal protein S3 | RPS3 | FLJ27450; FLJ26283; |
| | | | | | (RPS3), mRNA. | | MGC87870 |
| 1168 | 1160 | 6180088 | NM_145230.2 | NM_145230 | ATPase, H+ | ATP6V0E2 | ATP6V0E2L; C7orf32 |
| | | | | | transporting V0 subunit | | |
| | | | | | e2 (ATP6V0E2), | | |
| | | | | | transcript variant 1, | | |
| | | | | | mRNA. | | |
| 1169 | 1161 | 6180128 | NR_003089.1 | NR_003089 | OTU domain, ubiquitin | OTUB1 | OTU1; HSPC263; |
| | | | | | aldehyde binding 1 | | MGC4584; |
| | | | | | (OTUB1), transcript | | MGC111158; |
| | | | | | variant 2, transcribed | | FLJ20113; OTB1; |
| | | | | | RNA. | | FLJ40710 |
| 1170 | 1162 | 6180176 | NM_198970.1 | NM_198970 | amino-terminal | AES | AES-2; ESP1; GRG5; |
| | | | | | enhancer of split (AES), | | GRG; TLE5; AES-1 |
| | | | | | transcript variant 3, | | |
| | | | | | mRNA. | | |
| 1171 | 1163 | 6180187 | NM_153046.1 | NM_153046 | tudor domain | TDRD9 | FLJ36164; |
| | | | | | containing 9 (TDRD9), | | DKFZp434N0820; |
| | | | | | mRNA. | | C14orf75; |
| | | | | | | | MGC135025; HIG-1 |
| 1172 | 1164 | 6180408 | NM_015953.3 | NM_015953 | nitric oxide synthase | NOSIP | CGI-25 |
| | | | | | interacting protein | | |
| | | | | | (NOSIP), mRNA. | | |
| 1173 | 1165 | 6180538 | NM_001494.2 | NM_001494 | GDP dissociation | GDI2 | FLJ37352; FLJ16452; |
| | | | | | inhibitor 2 (GDI2), | | RABGDIB |
| | | | | | mRNA. | | |
| 1174 | 1166 | 6200019 | NM_002258.2 | NM_002258 | killer cell lectin-like | KLRB1 | hNKR-P1A; NKR-P1; |
| | | | | | receptor subfamily B, | | CLEC5B; CD161; |
| | | | | | member 1 (KLRB1), | | MGC138614; |
| | | | | | mRNA. | | NKRP1A; NKR-P1A; |
| | | | | | | | NKR |
| 1175 | 1167 | 6200053 | NM_005102.2 | NM_005102 | fasciculation and | FEZ2 | MGC117372; |
| | | | | | elongation protein zeta | | HUM3CL |
| | | | | | 2 (zygin II) (FEZ2), | | |
| | | | | | transcript variant 1, | | |
| | | | | | mRNA. | | |
| 1176 | 1168 | 6200086 | NM_002778.2 | NM_002778 | prosaposin (variant | PSAP | GLBA; SAP1; |
| | | | | | Gaucher disease and | | MGC110993; |
| | | | | | variant metachromatic | | FLJ00245 |
| | | | | | leukodystrophy) | | |
| | | | | | (PSAP), transcript | | |
| | | | | | variant 1, mRNA. | | |
| 1177 | 1169 | 6200315 | NM_001375.2 | NM_001375 | deoxyribonuclease II, | DNASE2 | DNASE2A; DNL; |
| | | | | | lysosomal (DNASE2), | | DNL2 |
| | | | | | mRNA. | | |
| 1178 | 1170 | 6200370 | NM_006865.2 | NM_006865 | leukocyte | LILRA3 | HM31; LIR-4; e3; |
| | | | | | immunoglobulin-like | | CD85E; ILT6; HM43; |
| | | | | | receptor, subfamily A | | LIR4 |
| | | | | | (without TM domain), | | |
| | | | | | member 3 (LILRA3), | | |
| | | | | | mRNA. | | |
| 1179 | 1171 | 6200577 | NM_182898.2 | NM_182898 | cAMP responsive | CREB5 | CRE-BPA |
| | | | | | element binding protein | | |
| | | | | | 5 (CREB5), transcript | | |
| | | | | | variant 1, mRNA. | | |
| 1180 | 1172 | 6200753 | NM_015055.2 | NM_015055 | SWAP-70 protein | SWAP70 | HSPC321; FLJ39540; |
| | | | | | (SWAP70), mRNA. | | KIAA0640; SWAP-70 |
| 1181 | 1173 | 6200768 | NM_000945.3 | NM_000945 | protein phosphatase 3 | PPP3R1 | CALNB1; CNB; CNB1 |
| | | | | | (formerly 2B), | | |
| | | | | | regulatory subunit B, | | |
| | | | | | alpha isoform | | |
| | | | | | (PPP3R1), mRNA. | | |
| 1182 | 1174 | 6220044 | NM_001418.3 | NM_001418 | eukaryotic translation | EIF4G2 | AAG1; NAT1; p97; |
| | | | | | initiation factor 4 | | FLJ41344; DAP5 |
| | | | | | gamma, 2 (EIF4G2), | | |
| | | | | | transcript variant 1, | | |
| | | | | | mRNA. | | |
| 1183 | 1175 | 6220086 | NM_001037494.1 | NM_001037494 | dynein, light chain, | DYNLL1 | PIN; DLC1; |
| | | | | | LC8-type 1 (DYNLL1), | | MGC126137; DLC8; |
| | | | | | transcript variant 1, | | DNCL1; MGC126138; |
| | | | | | mRNA. | | LC8a; hdlc1; LC8; |
| | | | | | | | DNCLC1 |
| 1184 | 1176 | 6220112 | NR_002569.1 | NR_002569 | small Cajal body- | SCARNA9 | Z32; mgU2-19/30 |
| | | | | | specific RNA 9 | | |
| | | | | | (SCARNA9) on | | |
| | | | | | chromosome 11. | | |
| 1185 | 1177 | 6220278 | NM_030927.1 | NM_030927 | tetraspanin 14 | TSPAN14 | MGC11352; |
| | | | | | (TSPAN14), mRNA. | | TM4SF14; DC- |
| | | | | | | | TM4F2 |
| 1186 | 1178 | 6220288 | NM_001198.2 | NM_001198 | PR domain containing | PRDM1 | MGC118922; |
| | | | | | 1, with ZNF domain | | BLIMP1; PRDI-BF1; |
| | | | | | (PRDM1), transcript | | MGC118925; |
| | | | | | variant 1, mRNA. | | MGC118924; |
| | | | | | | | MGC118923 |
| 1187 | 1179 | 6220300 | NM_004965.6 | NM_004965 | high-mobility group | HMGN1 | FLJ31471; |
| | | | | | nucleosome binding | | MGC104230; |
| | | | | | domain 1 (HMGN1), | | MGC117425; |
| | | | | | mRNA. | | FLJ27265; HMG14 |
| 1188 | 1180 | 6220372 | NM_182543.1 | NM_182543 | NOL1/NOP2/Sun | NSUN6 | 4933414E04Rik; |
| | | | | | domain family, member | | NOPD1; FLJ23743 |
| | | | | | 6 (NSUN6), mRNA. | | |
| 1189 | 1181 | 6220451 | NM_001151.2 | NM_001151 | solute carrier family 25 | SLC25A4 | PEO2; ANT1; PEO3; |
| | | | | | (mitochondrial carrier; | | T1; ANT |
| | | | | | adenine nucleotide | | |
| | | | | | translocator), member 4 | | |
| | | | | | (SLC25A4), nuclear | | |
| | | | | | gene encoding | | |
| | | | | | mitochondrial protein, | | |
| | | | | | mRNA. | | |
| 1190 | 1182 | 6250010 | NM_198281.2 | NM_198281 | GPRIN family member | GPRIN3 | GRIN3; FLJ42625 |
| | | | | | 3 (GPRIN3), mRNA. | | |
| 1191 | 1183 | 6250091 | NM_014943.3 | NM_014943 | zinc fingers and | ZHX2 | KIAA0854 |
| | | | | | homeoboxes 2 (ZHX2), | | |
| | | | | | mRNA. | | |
| 1192 | 1184 | 6250121 | NM_014043.2 | NM_014043 | chromatin modifying | CHMP2B | DKFZP564O123; |
| | | | | | protein 2B (CHMP2B), | | DMT1; CHMP2.5; |
| | | | | | mRNA. | | VPS2B; VPS2-2 |
| 1193 | 1185 | 6250382 | NM_015391.2 | NM_015391 | anaphase promoting | ANAPC13 | SWM1; APC13; |
| | | | | | complex subunit 13 | | DKFZP566D193m; |
| | | | | | (ANAPC13), mRNA. | | DKFZP566D193 |
| 1194 | 1186 | 6250685 | NM_002576.3 | NM_002576 | p21/Cdc42/Rac1- | PAK1 | MGC130001; |
| | | | | | activated kinase 1 | | MGC130000; |
| | | | | | (STE20 homolog, | | PAKalpha |
| | | | | | yeast) (PAK1), mRNA. | | |
| 1195 | 1188 | 6270022 | NM_002110.2 | NM_002110 | hemopoietic cell kinase | HCK | JTK9 |
| | | | | | (HCK), mRNA. | | |
| 1196 | 1189 | 6270114 | NM_172313.1 | NM_172313 | colony stimulating | CSF3R | GCSFR; CD114 |
| | | | | | factor 3 receptor | | |
| | | | | | (granulocyte) (CSF3R), | | |
| | | | | | transcript variant 4, | | |
| | | | | | mRNA. | | |
| 1197 | 1190 | 6270717 | NM_003093.1 | NM_003093 | small nuclear | SNRPC | FLJ20302 |
| | | | | | ribonucleoprotein | | |
| | | | | | polypeptide C | | |
| | | | | | (SNRPC), mRNA. | | |
| 1198 | 1191 | 6280086 | NM_006498.2 | NM_006498 | lectin, galactoside- | LGALS2 | HL14; MGC75071 |
| | | | | | binding, soluble, 2 | | |
| | | | | | (LGALS2), mRNA. | | |
| 1199 | 1192 | 6280092 | NM_024103.2 | NM_024103 | solute carrier family 25 | SLC25A23 | MGC2615; MCSC2; |
| | | | | | (mitochondrial carrier; | | SCaMC-3; APC2 |
| | | | | | phosphate carrier), | | |
| | | | | | member 23 | | |
| | | | | | (SLC25A23), nuclear | | |
| | | | | | gene encoding | | |
| | | | | | mitochondrial protein, | | |
| | | | | | mRNA. | | |
| 1200 | 1193 | 6280184 | NM_020379.2 | NM_020379 | mannosidase, alpha, | MAN1C1 | MAN1A3; pp6318; |
| | | | | | class 1C, member 1 | | MAN1C; HMIC |
| | | | | | (MAN1C1), mRNA. | | |
| 1201 | 1194 | 6280332 | NM_022059.1 | NM_022059 | chemokine (C—X—C | CXCL16 | SRPSOX; CXCLG16; |
| | | | | | motif) ligand 16 | | SR-PSOX |
| | | | | | (CXCL16), mRNA. | | |
| 1202 | 1195 | 6280482 | NM_016101.3 | NM_016101 | nuclear import 7 | NIP7 | HSPC031; FLJ10296; |
| | | | | | homolog (S. cerevisiae) | | CGI-37; KD93 |
| | | | | | (NIP7), mRNA. | | |
| 1203 | 1198 | 6290132 | NM_007278.1 | NM_007278 | GABA(A) receptor- | GABARAP | MM46; MGC120155; |
| | | | | | associated protein | | FLJ25768; |
| | | | | | (GABARAP), mRNA. | | MGC120154 |
| 1204 | 1199 | 6290187 | NM_001032279.1 | NM_001032279 | RCE1 homolog, prenyl | RCE1 | FACE2; RCE1B; |
| | | | | | protein peptidase (S. cerevisiae) | | RCE1A |
| | | | | | (RCE1), | | |
| | | | | | transcript variant 2, | | |
| | | | | | mRNA. | | |
| 1205 | 1200 | 6290400 | NM_000734.2 | NM_000734 | CD247 molecule | CD247 | CD3Q; CD3H; TCRZ; |
| | | | | | (CD247), transcript | | CD3Z; CD3-ZETA |
| | | | | | variant 2, mRNA. | | |
| 1206 | 1201 | 6330025 | NM_058219.2 | NM_058219 | exosome component 6 | EXOSC6 | hMtr3p; MTR3; p11; |
| | | | | | (EXOSC6), mRNA. | | EAP4; Mtr3p |
| 1207 | 1202 | 6330068 | NM_000528.2 | NM_000528 | mannosidase, alpha, | MAN2B1 | LAMAN; MANB |
| | | | | | class 2B, member 1 | | |
| | | | | | (MAN2B1), mRNA. | | |
| 1208 | 1203 | 6330176 | NM_018095.3 | NM_018095 | kelch repeat and BTB | KBTBD4 | FLJ10450; BKLHD4; |
| | | | | | (POZ) domain | | HSPC252 |
| | | | | | containing 4 (KBTBD4), | | |
| | | | | | transcript variant 1, | | |
| | | | | | mRNA. | | |
| 1209 | 1204 | 6330196 | NM_002371.2 | NM_002371 | mal, T-cell | MAL | — |
| | | | | | differentiation protein | | |
| | | | | | (MAL), transcript variant | | |
| | | | | | a, mRNA. | | |
| 1210 | 1205 | 6330224 | NM_139247.2 | NM_139247 | adenylatecyclase 4 | ADCY4 | — |
| | | | | | (ADCY4), mRNA. | | |
| 1211 | 1206 | 6330373 | NM_001959.2 | NM_001959 | eukaryotic translation | EEF1B2 | EEF1B; EF1B; |
| | | | | | elongation factor 1 beta | | EEF1B1 |
| | | | | | 2 (EEF1B2), transcript | | |
| | | | | | variant 1, mRNA. | | |
| 1212 | 1207 | 6330484 | NM_004079.3 | NM_004079 | cathepsin S (CTSS), | CTSS | MGC3886 |
| | | | | | mRNA. | | |
| 1213 | 1208 | 6330491 | NM_015979.2 | NM_015979 | mediator complex | MED23 | MED23; |
| | | | | | subunit 23 (MED23), | | DKFZp434H0117; |
| | | | | | transcript variant 2, | | CRSP3; DRIP130; |
| | | | | | mRNA. | | CRSP133; CRSP130; |
| | | | | | | | SUR2 |
| 1214 | 1209 | 6350131 | NM_001907.1 | NM_001907 | chymotrypsin-like | CTRL | CTRL1; MGC70821 |
| | | | | | (CTRL), mRNA. | | |
| 1215 | 1210 | 6370369 | NM_001040021.1 | NM_001040021 | CD14 molecule | CD14 | — |
| | | | | | (CD14), transcript | | |
| | | | | | variant 2, mRNA. | | |
| 1216 | 1211 | 6370468 | NM_001031827.1 | NM_001031827 | bolA homolog 2 (E. coli) | BOLA2 | BOLA2A; My016 |
| | | | | | (BOLA2), mRNA. | | |
| 1217 | 1212 | 6370593 | NM_138707.1 | NM_138707 | B-cell CLL/lymphoma | BCL7B | — |
| | | | | | 7B (BCL7B), transcript | | |
| | | | | | variant 2, mRNA. | | |
| 1218 | 1213 | 6370661 | NM_004069.3 | NM_004069 | adaptor-related protein | AP2S1 | AP17; CLAPS2; |
| | | | | | complex 2, sigma 1 | | AP17-DELTA |
| | | | | | subunit (AP2S1), | | |
| | | | | | transcript variant AP17, | | |
| | | | | | mRNA. | | |
| 1219 | 1214 | 6380093 | NM_001031696.1 | NM_001031696 | phospholipase D family, | PLD3 | HU-K4 |
| | | | | | member 3 (PLD3), | | |
| | | | | | transcript variant 1, | | |
| | | | | | mRNA. | | |
| 1220 | 1215 | 6380110 | NM_022006.1 | NM_022006 | FXYD domain | FXYD7 | FLJ25096 |
| | | | | | containing ion transport | | |
| | | | | | regulator 7 (FXYD7), | | |
| | | | | | mRNA. | | |
| 1221 | 1216 | 6380161 | NM_000061.1 | NM_000061 | Brutonagammaglobulin | BTK | MGC126261; ATK; |
| | | | | | emia tyrosine kinase | | XLA; IMD1; AT; BPK; |
| | | | | | (BTK), mRNA. | | AGMX1; PSCTK1; |
| | | | | | | | MGC126262 |
| 1222 | 1217 | 6380445 | NM_006925.3 | NM_006925 | splicing factor, | SFRS5 | SRP40; HRS |
| | | | | | arginine/serine-rich 5 | | |
| | | | | | (SFRS5), transcript | | |
| | | | | | variant 2, mRNA. | | |
| 1223 | 1218 | 6380484 | NM_001002235.1 | NM_001002235 | serpin peptidase | SERPINA1 | PI1; MGC23330; |
| | | | | | inhibitor, clade A | | PRO2275; A1AT; |
| | | | | | (alpha-1 antiproteinase, | | AAT; MGC9222; PI; |
| | | | | | antitrypsin), member 1 | | A1A |
| | | | | | (SERPINA1), transcript | | |
| | | | | | variant 3, mRNA. | | |
| 1224 | 1219 | 6400243 | NM_001003407.1 | NM_001003407 | actin binding LIM | ABLIM1 | KIAA0059; FLJ14564; |
| | | | | | protein 1 (ABLIM1), | | MGC1224; LIMATIN; |
| | | | | | transcript variant 2, | | LIMAB1; |
| | | | | | mRNA. | | DKFZp781D0148; |
| | | | | | | | ABLIM |
| 1225 | 1221 | 6420040 | NM_144607.3 | NM_144607 | cytochrome b5 domain | CYB5D1 | FLJ32499 |
| | | | | | containing 1 (CYB5D1), | | |
| | | | | | mRNA. | | |
| 1226 | 1222 | 6420392 | NM_001014446.1 | NM_001014446 | OCIA domain | OCIAD2 | DKFZp686C03164; |
| | | | | | containing 2 (OCIAD2), | | MGC45416 |
| | | | | | transcript variant 1, | | |
| | | | | | mRNA. | | |
| 1227 | 1223 | 6420403 | NM_001013254.1 | NM_001013254 | lymphocyte-specific | LSP1 | WP34; pp52 |
| | | | | | protein 1 (LSP1), | | |
| | | | | | transcript variant 3, | | |
| | | | | | mRNA. | | |
| 1228 | 1224 | 6420746 | NM_002010.1 | NM_002010 | fibroblast growth factor | FGF9 | MGC119914; GAF; |
| | | | | | 9 (glia-activating factor) | | MGC119915; HBFG-9 |
| | | | | | (FGF9), mRNA. | | |
| 1229 | 1225 | 6450093 | NM_000952.3 | NM_000952 | platelet-activating factor | PTAFR | PAFR |
| | | | | | receptor (PTAFR), | | |
| | | | | | mRNA. | | |
| 1230 | 1226 | 6450129 | NM_004867.3 | NM_004867 | integral membrane | ITM2A | E25A; BRICD2A |
| | | | | | protein 2A (ITM2A), | | |
| | | | | | mRNA. | | |
| 1231 | 1227 | 6450139 | NR_002201.1 | NR_002201 | ferritin, heavy | FTHL3 | — |
| | | | | | polypeptide-like 3 | | |
| | | | | | (FTHL3) on | | |
| | | | | | chromosome 2. | | |
| 1232 | 1228 | 6450475 | NM_024640.3 | NM_024640 | yrdC domain containing | YRDC | FLJ23476; IRIP; |
| | | | | | (E. coli) (YRDC), | | FLJ26165; RP11- |
| | | | | | mRNA. | | 109P14.4; SUA5; |
| | | | | | | | DRIP3 |
| 1233 | 1229 | 6450538 | NM_006706.3 | NM_006706 | transcription elongation | TCERG1 | MGC133200; TAF2S; |
| | | | | | regulator 1 (TCERG1), | | CA150 |
| | | | | | transcript variant 1, | | |
| | | | | | mRNA. | | |
| 1234 | 1230 | 6450661 | NM_032250.1 | NM_032250 | ankyrin repeat domain | ANKRD20A1 | ANKRD20A; |
| | | | | | 20 family, member A1 | | DKFZp434A171 |
| | | | | | (ANKRD20A1), mRNA. | | |
| 1235 | 1231 | 6480059 | NM_001613.1 | NM_001613 | actin, alpha 2, smooth | ACTA2 | ACTSA |
| | | | | | muscle, aorta (ACTA2), | | |
| | | | | | mRNA. | | |
| 1236 | 1232 | 6480196 | NM_018070.3 | NM_018070 | single stranded DNA | SSBP3 | CSDP; FLJ10355; |
| | | | | | binding protein 3 | | SSDP1; SSDP |
| | | | | | (SSBP3), transcript | | |
| | | | | | variant 2, mRNA. | | |
| 1237 | 1233 | 6480349 | NM_012446.2 | NM_012446 | single-stranded DNA | SSBP2 | HSPC116; |
| | | | | | binding protein 2 | | DKFZp686F03273 |
| | | | | | (SSBP2), mRNA. | | |
| 1238 | 1234 | 6480360 | NM_138636.2 | NM_138636 | toll-like receptor 8 | TLR8 | MGC119600; |
| | | | | | (TLR8), transcript | | MGC119599 |
| | | | | | variant 2, mRNA. | | |
| 1239 | 1235 | 6510026 | NM_016584.2 | NM_016584 | interleukin 23, alpha | IL23A | IL-23A; IL23P19; |
| | | | | | subunit p19 (IL23A), | | MGC79388; SGRF; |
| | | | | | mRNA. | | P19; IL-23 |
| 1240 | 1236 | 6510279 | NM_001047434.1 | NM_001047434 | DPH3, KTI11 homolog | DPH3 | MGC20197; DPH3A; |
| | | | | | (S. cerevisiae) (DPH3), | | DELGIP1; ZCSL2; |
| | | | | | transcript variant 2, | | DELGIP; KTI11; |
| | | | | | mRNA. | | DESR1 |
| 1241 | 1237 | 6510367 | NM_002228.3 | NM_002228 | jun oncogene (JUN), | JUN | AP1; c-Jun |
| | | | | | mRNA. | | |
| 1242 | 1238 | 6510553 | NM_005224.2 | NM_005224 | AT rich interactive | ARID3A | E2FBP1; BRIGHT; |
| | | | | | domain 3A (BRIGHT- | | DRIL3; DRIL1 |
| | | | | | like) (ARID3A), mRNA. | | |
| 1243 | 1239 | 6510603 | NM_000485.2 | NM_000485 | adenine | APRT | MGC125857; AMP; |
| | | | | | phosphoribosyltransferase | | MGC125856; |
| | | | | | (APRT), transcript | | DKFZp686D13177; |
| | | | | | variant 1, mRNA. | | MGC129961 |
| 1244 | 1240 | 6520180 | NM_003853.2 | NM_003853 | interleukin 18 receptor | IL18RAP | MGC120590; |
| | | | | | accessory protein | | MGC120589; |
| | | | | | (IL18RAP), mRNA. | | CDw218b; CD218b; |
| | | | | | | | ACPL |
| 1245 | 1241 | 6520192 | NM_001039673.1 | NM_001039673 | Yip1 interacting factor | YIF1B | FinGER8 |
| | | | | | homolog B (S. cerevisiae) | | |
| | | | | | (YIF1B), | | |
| | | | | | transcript variant 4, | | |
| | | | | | mRNA. | | |
| 1246 | 1242 | 6520280 | NM_023926.3 | NM_023926 | zinc finger and SCAN | ZSCAN18 | ZNF447; |
| | | | | | domain containing 18 | | DKFZp586B1122; |
| | | | | | (ZSCAN18), mRNA. | | FLJ44152; MGC2427; |
| | | | | | | | MGC8682; |
| | | | | | | | MGC4074; FLJ12895 |
| 1247 | 1243 | 6520576 | NM_212552.2 | NM_212552 | bolA homolog 3 (E. coli) | BOLA3 | — |
| | | | | | (BOLA3), transcript | | |
| | | | | | variant 1, mRNA. | | |
| 1248 | 1244 | 6520630 | NM_181504.2 | NM_181504 | phosphoinositide-3- | PIK3R1 | p85; p85-ALPHA; |
| | | | | | kinase, regulatory | | GRB1 |
| | | | | | subunit 1 (alpha) | | |
| | | | | | (PIK3R1), transcript | | |
| | | | | | variant 2, mRNA. | | |
| 1249 | 1245 | 6520646 | NM_003870.3 | NM_003870 | IQ motif containing | IQGAP1 | HUMORFA01; SAR1; |
| | | | | | GTPase activating | | KIAA0051; p195 |
| | | | | | protein 1 (IQGAP1), | | |
| | | | | | mRNA. | | |
| 1250 | 1246 | 6550064 | NM_181738.1 | NM_181738 | peroxiredoxin 2 | PRDX2 | TDPX1; MGC4104; |
| | | | | | (PRDX2), nuclear gene | | TSA; PRP; PRX2; |
| | | | | | encoding mitochondrial | | NKEFB; PRXII |
| | | | | | protein, transcript | | |
| | | | | | variant 3, mRNA. | | |
| 1251 | 1247 | 6550673 | NM_001152.1 | NM_001152 | solute carrier family 25 | SLC25A5 | T3; 2F1; ANT2; T2 |
| | | | | | (mitochondrial carrier; | | |
| | | | | | adenine nucleotide | | |
| | | | | | translocator), member 5 | | |
| | | | | | (SLC25A5), mRNA. | | |
| 1252 | 1248 | 6550754 | NM_016337.2 | NM_016337 | Enah/Vasp-like (EVL), | EVL | RNB6 |
| | | | | | mRNA. | | |
| 1253 | 1249 | 6560136 | NM_001042442.1 | NM_001042442 | calpastatin (CAST), | CAST | MGC9402; BS-17 |
| | | | | | transcript variant 8, | | |
| | | | | | mRNA. | | |
| 1254 | 1250 | 6560180 | NM_001003696.1 | NM_001003696 | ATP synthase, H+ | ATP5J | ATP5A CF6; F6; |
| | | | | | transporting, | | ATPM; ATP5 |
| | | | | | mitochondrial F0 | | |
| | | | | | complex, subunit F6 | | |
| | | | | | (ATP5J), nuclear gene | | |
| | | | | | encoding mitochondrial | | |
| | | | | | protein, transcript | | |
| | | | | | variant 3, mRNA. | | |
| 1255 | 1251 | 6560300 | NM_001860.2 | NM_001860 | solute carrier family 31 | SLC31A2 | hCTR2; CTR2; |
| | | | | | (copper transporters), | | COPT2 |
| | | | | | member 2 (SLC31A2), | | |
| | | | | | mRNA. | | |
| 1256 | 1252 | 6560451 | NM_138394.2 | NM_138394 | heterogeneous nuclear | HNRPLL | SRRF |
| | | | | | ribonucleoprotein L-like | | |
| | | | | | (HNRPLL), mRNA. | | |
| 1257 | 1253 | 6580041 | NM_006433.2 | NM_006433 | granulysin (GNLY), | GNLY | D2S69E; 519; LAG2; |
| | | | | | transcript variant | | LAG-2; TLA519; |
| | | | | | NKG5, mRNA. | | NKG5 |
| 1258 | 1254 | 6580059 | NM_003355.2 | NM_003355 | uncoupling protein 2 | UCP2 | SLC25A8; UCPH |
| | | | | | (mitochondrial, proton | | |
| | | | | | carrier) (UCP2), nuclear | | |
| | | | | | gene encoding | | |
| | | | | | mitochondrial protein, | | |
| | | | | | mRNA. | | |
| 1259 | 1255 | 6580437 | NM_012139.2 | NM_012139 | secretion regulating | SERGEF | Gnefr; DELGEF |
| | | | | | guanine nucleotide | | |
| | | | | | exchange factor | | |
| | | | | | (SERGEF), mRNA. | | |
| 1260 | 1257 | 6580717 | NM_032265.1 | NM_032265 | zinc finger, MYND-type | ZMYND15 | DKFZp434N127 |
| | | | | | containing 15 | | |
| | | | | | (ZMYND15), mRNA. | | |
| 1261 | 1258 | 6580750 | NM_002621.1 | NM_002621 | complement factor | CFP | PFC; PROPERDIN; |
| | | | | | properdin (CFP), | | BFD; PFD |
| | | | | | mRNA. | | |
| 1262 | 1259 | 6590201 | NM_001183.4 | NM_001183 | ATPase, H+ | ATP6AP1 | XAP-3; CF2; 16A; |
| | | | | | transporting, lysosomal | | ATP6IP1; ATP6S1; |
| | | | | | accessory protein 1 | | Ac45; MGC129781; |
| | | | | | (ATP6AP1), mRNA. | | XAP3; VATPS1 |
| 1263 | 1260 | 6590441 | NM_004238.1 | NM_004238 | thyroid hormone | TRIP12 | KIAA0045; |
| | | | | | receptor interactor 12 | | MGC138850; |
| | | | | | (TRIP12), mRNA. | | MGC138849 |
| 1264 | 1261 | 6590722 | NM_016374.5 | NM_016374 | AT rich interactive | ARID4B | BCM; RBP1L1; |
| | | | | | domain 4B (RBP1-like) | | MGC163290; |
| | | | | | (ARID4B), transcript | | BRCAA1; SAP180; |
| | | | | | variant 1, mRNA. | | RBBP1L1; |
| | | | | | | | DKFZp313M2420 |
| 1265 | 1262 | 6620072 | NM_004930.1 | NM_004930 | capping protein (actin | CAPZB | CAPB; MGC104401; |
| | | | | | filament) muscle Z-line, | | MGC129749; |
| | | | | | beta (CAPZB), mRNA. | | MGC129750; |
| | | | | | | | CAPPB; CAPZ |
| 1266 | 1263 | 6620170 | NM_000517.3 | NM_000517 | hemoglobin, alpha 2 | HBA2 | HBA1 |
| | | | | | (HBA2), mRNA. | | |
| 1267 | 1264 | 6620201 | NM_017644.3 | NM_017644 | kelch-like 24 | KLHL24 | FLJ25796; KRIP6; |
| | | | | | (Drosophila) (KLHL24), | | DRE1 |
| | | | | | mRNA. | | |
| 1268 | 1265 | 6620315 | NM_182476.1 | NM_182476 | coenzyme Q6 | COQ6 | CGI-10 |
| | | | | | homolog, | | |
| | | | | | monooxygenase (S. cerevisiae) | | |
| | | | | | (COQ6), | | |
| | | | | | transcript variant 1, | | |
| | | | | | mRNA. | | |
| 1269 | 1266 | 6620474 | NM_138924.1 | NM_138924 | guanidinoacetate N- | GAMT | TP53I2; PIG2 |
| | | | | | methyltransferase | | |
| | | | | | (GAMT), transcript | | |
| | | | | | variant 2, mRNA. | | |
| 1270 | 1267 | 6650161 | NM_006572.3 | NM_006572 | guanine nucleotide | GNA13 | MGC46138; G13 |
| | | | | | binding protein (G | | |
| | | | | | protein), alpha 13 | | |
| | | | | | (GNA13), mRNA. | | |
| 1271 | 1268 | 6650242 | NM_021034.2 | NM_021034 | interferon induced | IFITM3 | 1-8U; IP15 |
| | | | | | transmembrane protein | | |
| | | | | | 3 (1-8U) (IFITM3), | | |
| | | | | | mRNA. | | |
| 1272 | 1269 | 6650639 | NM_199245.1 | NM_199245 | vesicle-associated | VAMP1 | SYB1; VAMP-1; |
| | | | | | membrane protein 1 | | DKFZp686H12131 |
| | | | | | (synaptobrevin 1) | | |
| | | | | | (VAMP1), transcript | | |
| | | | | | variant 2, mRNA. | | |
| 1273 | 1270 | 6650746 | NM_001040456.1 | NM_001040456 | rhomboid domain | RHBDD2 | NPD007; RHBDL7 |
| | | | | | containing 2 | | |
| | | | | | (RHBDD2), transcript | | |
| | | | | | variant 1, mRNA. | | |
| 1274 | 1271 | 6660092 | NM_001745.2 | NM_001745 | calcium modulating | CAMLG | CAML; MGC163197 |
| | | | | | ligand (CAMLG), | | |
| | | | | | mRNA. | | |
| 1275 | 1272 | 6660343 | NM_006328.2 | NM_006328 | RNA binding motif | RBM14 | SYTIP1; COAA; SIP; |
| | | | | | protein 14 (RBM14), | | DKFZp779J0927 |
| | | | | | mRNA. | | |
| 1276 | 1273 | 6660368 | NM_001042600.1 | NM_001042600 | mitogen-activated | MAP4K1 | HPK1 |
| | | | | | protein kinase | | |
| | | | | | kinasekinasekinase 1 | | |
| | | | | | (MAP4K1), transcript | | |
| | | | | | variant 1, mRNA. | | |
| 1277 | 1274 | 6660398 | NM_002003.2 | NM_002003 | ficolin | FCN1 | FCNM |
| | | | | | (collagen/fibrinogen | | |
| | | | | | domain containing) 1 | | |
| | | | | | (FCN1), mRNA. | | |
| 1278 | 1275 | 6660411 | NM_018466.3 | NM_018466 | asparagine-linked | ALG13 | YGL047W; GLT28D1; |
| | | | | | glycosylation 13 | | MDS031; CXorf45 |
| | | | | | homolog (S. cerevisiae) | | |
| | | | | | (ALG13), mRNA. | | |
| 1279 | 1276 | 6660475 | NM_001042729.1 | NM_001042729 | Gardner-Rasheed | FGR | p58c-fgr, SRC2; c-fgr; |
| | | | | | feline sarcoma viral (v- | | FLJ43153; |
| | | | | | fgr) oncogene homolog | | MGC75096; c-src2; |
| | | | | | (FGR), transcript | | p55c-fgr |
| | | | | | variant 3, mRNA. | | |
| 1280 | 1277 | 6660673 | NM_016628.2 | NM_016628 | WW domain containing | WAC | bA48B24; |
| | | | | | adaptor with coiled-coil | | MGC10753; BM-016; |
| | | | | | (WAC), transcript | | Wwp4; PRO1741; |
| | | | | | variant 1, mRNA. | | bA48B24.1 |
| 1281 | 1278 | 6660768 | NM_182972.2 | NM_182972 | interferon regulatory | IRF2BP2 | MGC72189 |
| | | | | | factor 2 binding protein | | |
| | | | | | 2 (IRF2BP2), transcript | | |
| | | | | | variant 1, mRNA. | | |
| 1282 | 1279 | 6760273 | NM_020926.2 | NM_020926 | BCL6 co-repressor | BCOR | MGC131961; |
| | | | | | (BCOR), transcript | | KIAA1575; FLJ20285; |
| | | | | | variant 2, mRNA. | | MGC71031; |
| | | | | | | | MCOPS2; FLJ38041; |
| | | | | | | | MAA2; ANOP2 |
| 1283 | 1280 | 6760315 | NM_006768.2 | NM_006768 | BRCA1 associated | BRAP | RNF52; IMP; BRAP2 |
| | | | | | protein (BRAP), mRNA. | | |
| 1284 | 1282 | 6770377 | NM_022918.2 | NM_022918 | transmembrane protein | TMEM135 | FLJ22104; |
| | | | | | 135 (TMEM135), | | DKFZp686I1974 |
| | | | | | mRNA. | | |
| 1285 | 1283 | 6770474 | NM_030622.6 | NM_030622 | cytochrome P450, | CYP2S1 | — |
| | | | | | family 2, subfamily S, | | |
| | | | | | polypeptide 1 | | |
| | | | | | (CYP2S1), mRNA. | | |
| 1286 | 1284 | 6770754 | NM_031454.1 | NM_031454 | selenoprotein O | SELO | MGC131879 |
| | | | | | (SELO), mRNA. | | |
| 1287 | 1285 | 6840072 | NM_000314.4 | NM_000314 | phosphatase and | PTEN | MMAC1; MHAM; |
| | | | | | tensin homolog | | TEP1; BZS; PTEN1; |
| | | | | | (mutated in multiple | | MGC11227 |
| | | | | | advanced cancers 1) | | |
| | | | | | (PTEN), mRNA. | | |
| 1288 | 1286 | 6840184 | NM_002087.2 | NM_002087 | granulin (GRN), mRNA. | GRN | PEPI; PGRN; |
| | | | | | | | PCDGF; GEP; GP88 |
| 1289 | 1287 | 6840246 | NM_000308.2 | NM_000308 | cathepsin A (CTSA), | CTSA | NGBE; PPCA; GSL; |
| | | | | | mRNA. | | PPGB; GLB2 |
| 1290 | 1288 | 6840408 | NM_004271.3 | NM_004271 | lymphocyte antigen 86 | LY86 | dJ80N2.1; MMD-1; |
| | | | | | (LY86), mRNA. | | RP1-80N2.1; MD-1 |
| 1291 | 1289 | 6840593 | NM_031905.2 | NM_031905 | armadillo repeat | ARMC10 | SVH; MGC3195; |
| | | | | | containing 10 | | PNAS-112 |
| | | | | | (ARMC10), mRNA. | | |
| 1292 | 1290 | 6860095 | NM_205835.2 | NM_205835 | lipolysis stimulated | LSR | MGC48312; LISCH7; |
| | | | | | lipoprotein receptor | | MGC48503; |
| | | | | | (LSR), transcript variant | | MGC10659 |
| | | | | | 3, mRNA. | | |
| 1293 | 1291 | 6860202 | NM_001064.1 | NM_001064 | transketolase | TKT | TKT1; FLJ34765 |
| | | | | | (Wernicke-Korsakoff | | |
| | | | | | syndrome) (TKT), | | |
| | | | | | mRNA. | | |
| 1294 | 1292 | 6860452 | NM_002598.2 | NM_002598 | programmed cell death | PDCD2 | RP8; MGC12347; |
| | | | | | 2 (PDCD2), transcript | | ZMYND7 |
| | | | | | variant 1, mRNA. | | |
| 1295 | 1293 | 6860553 | NM_003136.2 | NM_003136 | signal recognition | SRP54 | — |
| | | | | | particle 54 kDa | | |
| | | | | | (SRP54), mRNA. | | |
| 1296 | 1294 | 6860678 | NR_003249.1 | NR_003249 | heterogeneous nuclear | HNRPDL | JKTBP2; JKTBP; |
| | | | | | ribonucleoprotein D-like | | laAUF1; HNRNP |
| | | | | | (HNRPDL), transcript | | |
| | | | | | variant 3, transcribed | | |
| | | | | | RNA. | | |
| 1297 | 1295 | 6900025 | NM_004805.2 | NM_004805 | polymerase (RNA) II | POLR2D | HSRPB4; RBP4; |
| | | | | | (DNA directed) | | HSRBP4 |
| | | | | | polypeptide D | | |
| | | | | | (POLR2D), mRNA. | | |
| 1298 | 1296 | 6900398 | NM_002004.2 | NM_002004 | farnesyldiphosphate | FDPS | FPS |
| | | | | | synthase (farnesyl | | |
| | | | | | pyrophosphate | | |
| | | | | | synthetase, | | |
| | | | | | dimethylallyltranstransferase, | | |
| | | | | | geranyltranstransferase) | | |
| | | | | | (FDPS), mRNA. | | |
| 1299 | 1297 | 6900593 | NM_001004491.1 | NM_001004491 | olfactory receptor, | OR2AK2 | OR1-47; OR2AK1P |
| | | | | | family 2, subfamily AK, | | |
| | | | | | member 2 (OR2AK2), | | |
| | | | | | mRNA. | | |
| 1300 | 1298 | 6900612 | NM_001011546.1 | NM_001011546 | destrin (actin | DSTN | ACTDP; bA462D18.2; |
| | | | | | depolymerizing factor) | | ADF |
| | | | | | (DSTN), transcript | | |
| | | | | | variant 2, mRNA. | | |
| 1301 | 1299 | 6900630 | NM_001684.3 | NM_001684 | ATPase, Ca++ | ATP2B4 | DKFZp686M088; |
| | | | | | transporting, plasma | | PMCA4b; |
| | | | | | membrane 4 | | DKFZp686G08106; |
| | | | | | (ATP2B4), transcript | | PMCA4x; PMCA4; |
| | | | | | variant 2, mRNA. | | ATP2B2; MXRA1 |
| 1302 | 1300 | 6900674 | NM_017664.2 | NM_017664 | ankyrin repeat domain | ANKRD10 | DKFZp686B07190; |
| | | | | | 10 (ANKRD10), mRNA. | | FLJ20093 |
| 1303 | 1301 | 6940039 | NM_003105.3 | NM_003105 | sortilin-related receptor, | SORL1 | LR11; LRP9; SORLA; |
| | | | | | L(DLR class) A | | SorLA-1; gp250 |
| | | | | | repeats-containing | | |
| | | | | | (SORL1), mRNA. | | |
| 1304 | 1302 | 6940066 | NM_005022.2 | NM_005022 | profilin 1 (PFN1), | PFN1 | — |
| | | | | | mRNA. | | |
| 1305 | 1303 | 6940255 | NM_020360.2 | NM_020360 | phospholipid | PLSCR3 | — |
| | | | | | scramblase 3 | | |
| | | | | | (PLSCR3), mRNA. | | |
| 1306 | 1304 | 6960072 | NM_002156.4 | NM_002156 | heat shock 60 kDa | HSPD1 | HuCHA60; GROEL; |
| | | | | | protein 1 (chaperonin) | | HSP60; SPG13; |
| | | | | | (HSPD1), nuclear gene | | CPN60; HSP65 |
| | | | | | encoding mitochondrial | | |
| | | | | | protein, transcript | | |
| | | | | | variant 1, mRNA. | | |
| 1307 | 1305 | 6960129 | NM_152729.2 | NM_152729 | 5′-nucleotidase domain | NT5DC1 | MGC131837; |
| | | | | | containing 1 (NT5DC1), | | C6orf200; |
| | | | | | mRNA. | | MGC24302; LP2642; |
| | | | | | | | NT5C2L1 |
| 1308 | 1306 | 6960168 | NM_020452.2 | NM_020452 | ATPase, class I, type | ATP8B2 | ATPID; |
| | | | | | 8B, member 2 | | DKFZp434M0219; |
| | | | | | (ATP8B2), transcript | | KIAA1137 |
| | | | | | variant 1, mRNA. | | |
| 1309 | 1307 | 6960242 | NM_019048.1 | NM_019048 | asparagine synthetase | ASNSD1 | NBLA00058; |
| | | | | | domain containing 1 | | NS3TP1; FLJ20752 |
| | | | | | (ASNSD1), mRNA. | | |
| 1310 | 1308 | 6960440 | NM_181762.1 | NM_181762 | ubiquitin-conjugating | UBE2A | UBC2; RAD6A; |
| | | | | | enzyme E2A (RAD6 | | HHR6A |
| | | | | | homolog) (UBE2A), | | |
| | | | | | transcript variant 2, | | |
| | | | | | mRNA. | | |
| 1311 | 1310 | 6960768 | NM_006963.3 | NM_006963 | zinc finger protein 22 | ZNF22 | HKR-T1; Zfp422; |
| | | | | | (KOX 15) (ZNF22), | | KOX15; ZNF422 |
| | | | | | mRNA. | | |
| 1312 | 1311 | 6980164 | NM_017595.4 | NM_017595 | NFKB inhibitor | NKIRAS2 | KBRAS2; |
| | | | | | interacting Ras-like 2 | | MGC74742; kappaB- |
| | | | | | (NKIRAS2), transcript | | Ras2; |
| | | | | | variant 2, mRNA. | | DKFZP434N1526 |
| 1313 | 1312 | 6980471 | NM_130436.2 | NM_130436 | dual-specificity tyrosine- | DYRK1A | HP86; DYRK; |
| | | | | | (Y)-phosphorylation | | DYRK1; MNBH; MNB |
| | | | | | regulated kinase 1A | | |
| | | | | | (DYRK1A), transcript | | |
| | | | | | variant 2, mRNA. | | |
| 1314 | 1313 | 6980541 | NM_003255.4 | NM_003255 | TIMP metallopeptidase | TIMP2 | CSC-21K |
| | | | | | inhibitor 2 (TIMP2), | | |
| | | | | | mRNA. | | |
| 1315 | 1314 | 7000121 | NM_176877.2 | NM_176877 | InaD-like (Drosophila) | INADL | Cipp; FLJ26982; |
| | | | | | (INADL), mRNA. | | PATJ |
| 1316 | 1315 | 7000307 | NM_004458.1 | NM_004458 | acyl-CoA synthetase | ACSL4 | MRX68; FACL4; |
| | | | | | long-chain family | | ACS4; MRX63; |
| | | | | | member 4 (ACSL4), | | LACS4 |
| | | | | | transcript variant 1, | | |
| | | | | | mRNA. | | |
| 1317 | 1317 | 7040707 | NM_015074.2 | NM_015074 | kinesin family member | KIF1B | KIAA0591; HMSNII; |
| | | | | | 1B (KIF1B), transcript | | CMT2; FLJ23699; |
| | | | | | variant 1, mRNA. | | KIAA1448; CMT2A; |
| | | | | | | | MGC134844; |
| | | | | | | | CMT2A1; KLP |
| 1318 | 1318 | 7040709 | NM_006548.4 | NM_006548 | insulin-like growth | IGF2BP2 | IMP-2; VICKZ2; p62; |
| | | | | | factor 2 mRNA binding | | IMP2 |
| | | | | | protein 2 (IGF2BP2), | | |
| | | | | | transcript variant 1, | | |
| | | | | | mRNA. | | |
| 1319 | 1319 | 7040735 | NM_000784.2 | NM_000784 | cytochrome P450, | CYP27A1 | CYP27; CTX; CP27 |
| | | | | | family 27, subfamily A, | | |
| | | | | | polypeptide 1 | | |
| | | | | | (CYP27A1), nuclear | | |
| | | | | | gene encoding | | |
| | | | | | mitochondrial protein, | | |
| | | | | | mRNA. | | |
| 1320 | 1320 | 7050189 | NM_014502.3 | NM_014502 | PRP19/PSO4 pre- | PRPF19 | NMP200; PRP19; |
| | | | | | mRNA processing | | hPSO4; UBOX4; |
| | | | | | factor 19 homolog (S. cerevisiae) | | SNEV; PSO4 |
| | | | | | (PRPF19), | | |
| | | | | | mRNA. | | |
| 1321 | 1321 | 7050332 | NM_005348.2 | NM_005348 | heat shock protein | HSP90AA1 | HSPCAL4; HSPN; |
| | | | | | 90 kDa alpha | | HSP86; Hsp89; |
| | | | | | (cytosolic), class A | | HSP90N; HSPC1; |
| | | | | | member 1 | | HSP90A; HSPCAL1; |
| | | | | | (HSP90AA1), transcript | | Hsp90; HSPCA; |
| | | | | | variant 2, mRNA. | | LAP2; FLJ31884 |
| 1322 | 1322 | 7050364 | NM_173849.2 | NM_173849 | goosecoidhomeobox | GSC | — |
| | | | | | (GSC), mRNA. | | |
| 1323 | 1323 | 7050494 | NM_014713.3 | NM_014713 | lysosomal-associated | LAPTM4A | LAPTM4; KIAA0108; |
| | | | | | protein transmembrane | | Mtrp; HUMORF13; |
| | | | | | 4 alpha (LAPTM4A), | | MBNT |
| | | | | | mRNA. | | |
| 1324 | 1324 | 7050619 | NM_017724.1 | NM_017724 | leucine rich repeat (in | LRRFIP2 | FLJ20248; HUFI-2; |
| | | | | | FLII) interacting protein | | FLJ22683; |
| | | | | | 2 (LRRFIP2), transcript | | DKFZp434H2035 |
| | | | | | variant 2, mRNA. | | |
| 1325 | 1325 | 7050717 | NM_004267.3 | NM_004267 | carbohydrate (N- | CHST2 | C6ST |
| | | | | | acetylglucosamine-6-O) | | |
| | | | | | sulfotransferase 2 | | |
| | | | | | (CHST2), mRNA. | | |
| 1326 | 1326 | 7100156 | NM_203504.1 | NM_203504 | GTPase activating | G3BP2 | — |
| | | | | | protein (SH3 domain) | | |
| | | | | | binding protein 2 | | |
| | | | | | (G3BP2), transcript | | |
| | | | | | variant 3, mRNA. | | |
| 1327 | 1327 | 7100338 | NM_001032278.1 | NM_001032278 | matrix metallopeptidase | MMP28 | MMP25; MM28 |
| | | | | | 28 (MMP28), transcript | | |
| | | | | | variant 3, mRNA. | | |
| 1328 | 1328 | 7100520 | NM_006368.4 | NM_006368 | cAMP responsive | CREB3 | LZIP; LUMAN; |
| | | | | | element binding protein | | MGC15333; |
| | | | | | 3 (CREB3), mRNA. | | MGC19782 |
| 1329 | 1331 | 7160059 | NM_004047.3 | NM_004047 | ATPase, H+ | ATP6V0B | HATPL; ATP6F; |
| | | | | | transporting, lysosomal | | VMA16 |
| | | | | | 21 kDa, V0 subunit b | | |
| | | | | | (ATP6V0B), transcript | | |
| | | | | | variant 1, mRNA. | | |
| 1330 | 1332 | 7160382 | NM_181713.3 | NM_181713 | UBX domain containing | UBXD4 | MGC138202 |
| | | | | | 4 (UBXD4), mRNA. | | |
| 1331 | 1333 | 7160593 | NM_152542.2 | NM_152542 | protein phosphatase 1K | PPM1K | UG0882E07; PP2Cm; |
| | | | | | (PP2C domain | | DKFZp667B084; |
| | | | | | containing) (PPM1K), | | PTMP; |
| | | | | | mRNA. | | DKFZp761G058 |
| 1332 | 1334 | 7200041 | NM_000310.2 | NM_000310 | palmitoyl-protein | PPT1 | CLN1; INCL; PPT |
| | | | | | thioesterase 1 (ceroid- | | |
| | | | | | lipofuscinosis, neuronal | | |
| | | | | | 1, infantile) (PPT1), | | |
| | | | | | mRNA. | | |
| 1333 | 1335 | 7200142 | NM_032940.2 | NM_032940 | polymerase (RNA) II | POLR2C | RPB31; hsRPB3; |
| | | | | | (DNA directed) | | RPB3; hRPB33 |
| | | | | | polypeptide C, 33 kDa | | |
| | | | | | (POLR2C), mRNA. | | |
| 1334 | 1336 | 7200242 | NM_018837.2 | NM_018837 | sulfatase 2 (SULF2), | SULF2 | MGC126411; |
| | | | | | transcript variant 1, | | FLJ90554; KIAA1247; |
| | | | | | mRNA. | | HSULF-2; |
| | | | | | | | DKFZp313E091 |
| 1335 | 1337 | 7200392 | NM_001212.3 | NM_001212 | complement | C1QBP | gC1qR; HABP1; |
| | | | | | component 1, q | | SF2p32; p32; gC1Q- |
| | | | | | subcomponent binding | | R; GC1QBP |
| | | | | | protein (C1QBP), | | |
| | | | | | nuclear gene encoding | | |
| | | | | | mitochondrial protein, | | |
| | | | | | mRNA. | | |
| 1336 | 1338 | 7200435 | NM_005170.2 | NM_005170 | achaete-scute complex | ASCL2 | ASH2; MASH2; |
| | | | | | homolog 2 (Drosophila) | | HASH2 |
| | | | | | (ASCL2), mRNA. | | |
| 1337 | 1339 | 7200475 | NM_015698.3 | NM_015698 | G patch domain and | GPKOW | GPATCH5; GPATC5; |
| | | | | | KOW motifs (GPKOW), | | T54 |
| | | | | | mRNA. | | |
| 1338 | 1340 | 7200601 | NM_001044390.1 | NM_001044390 | mucin 1, cell surface | MUC1 | PEMT; PUM; PEM; |
| | | | | | associated (MUC1), | | EMA; H23AG; MAM6; |
| | | | | | transcript variant 5, | | CD227 |
| | | | | | mRNA. | | |
| 1339 | 1341 | 7200670 | NM_016640.3 | NM_016640 | mitochondrial ribosomal | MRPS30 | PDCD9; MRP-S30; |
| | | | | | protein S30 (MRPS30), | | DKFZp566B2024; |
| | | | | | nuclear gene encoding | | PAP |
| | | | | | mitochondrial protein, | | |
| | | | | | mRNA. | | |
| 1340 | 1342 | 7210020 | NM_001006618.1 | NM_001006618 | mitogen-activated | MAPKAP1 | MGC2745; MIP1; |
| | | | | | protein kinase | | SIN1b; SIN1g; SIN1 |
| | | | | | associated protein 1 | | |
| | | | | | (MAPKAP1), transcript | | |
| | | | | | variant 6, mRNA. | | |
| 1341 | 1343 | 7210682 | NM_014716.2 | NM_014716 | centaurin, beta 1 | CENTB1 | KIAA0050; ACAP1 |
| | | | | | (CENTB1), mRNA. | | |
| 1342 | 1344 | 7210725 | NM_181463.1 | NM_181463 | mitochondrial ribosomal | MRPL55 | DKFZp686D1387; |
| | | | | | protein L55 (MRPL55), | | PRO19675; |
| | | | | | nuclear gene encoding | | MGC61802; |
| | | | | | mitochondrial protein, | | AAVG5835 |
| | | | | | transcript variant 5, | | |
| | | | | | mRNA. | | |
| 1343 | 1345 | 7320307 | NM_005707.1 | NM_005707 | programmed cell death | PDCD7 | HES18; ES18; |
| | | | | | 7 (PDCD7), mRNA. | | MGC22015 |
| 1344 | 1346 | 7320424 | NR_002944.2 | NR_002944 | heterogeneous nuclear | HNRPA1L-2 | — |
| | | | | | ribonucleoprotein A1 | | |
| | | | | | pseudogene | | |
| | | | | | (HNRPA1L-2) on | | |
| | | | | | chromosome 19. | | |
| 1345 | 1347 | 7320594 | NM_003461.4 | NM_003461 | zyxin (ZYX), transcript | ZYX | ESP-2; HED-2 |
| | | | | | variant 1, mRNA. | | |
| 1346 | 1348 | 7320687 | NM_002697.2 | NM_002697 | POU class 2 homeobox | POU2F1 | OCT1; OTF1 |
| | | | | | 1 (POU2F1), mRNA. | | |
| 1347 | 1349 | 7330068 | NM_031287.2 | NM_031287 | splicing factor 3b, | SF3B5 | SF3b10; MGC3133 |
| | | | | | subunit 5, 10 kDa | | |
| | | | | | (SF3B5), mRNA. | | |
| 1348 | 1351 | 7330504 | NM_138553.1 | NM_138553 | B-cell CLL/lymphoma | BCL11A | BCL11A-L; CTIP1; |
| | | | | | 11A (zinc finger protein) | | FLJ10173; BCL11A- |
| | | | | | (BCL11A), transcript | | XL; BCL11A-S; |
| | | | | | variant 5, mRNA. | | FLJ34997; KIAA1809; |
| | | | | | | | EVI9 |
| 1349 | 1352 | 7330544 | NM_005165.2 | NM_005165 | aldolase C, fructose- | ALDOC | ALDC |
| | | | | | bisphosphate | | |
| | | | | | (ALDOC), mRNA. | | |
| 1350 | 1353 | 7330671 | NM_145699.2 | NM_145699 | apolipoprotein B mRNA | APOBEC3A | PHRBN; bK150C2.1; |
| | | | | | editing enzyme, | | ARP3 |
| | | | | | catalytic polypeptide- | | |
| | | | | | like 3A (APOBEC3A), | | |
| | | | | | mRNA. | | |
| 1351 | 1354 | 7330753 | NM_005891.2 | NM_005891 | acetyl-Coenzyme A | ACAT2 | — |
| | | | | | acetyltransferase 2 | | |
| | | | | | (ACAT2), mRNA. | | |
| 1352 | 1355 | 7380164 | NM_004604.3 | NM_004604 | syntaxin 4 (STX4), | STX4 | STX4A; p35-2 |
| | | | | | mRNA. | | |
| 1353 | 1356 | 7380452 | NM_018203.1 | NM_018203 | kelch domain | KLHDC8A | FLJ10748 |
| | | | | | containing 8A | | |
| | | | | | (KLHDC8A), mRNA. | | |
| 1354 | 1358 | 7380626 | NM_019027.1 | NM_019027 | RNA-binding protein | FLJ20273 | DKFZp686F02235 |
| | | | | | (FLJ20273), mRNA. | | |
| 1355 | 1359 | 7400114 | NM_023039.2 | NM_023039 | ankyrin repeat, family A | ANKRA2 | ANKRA |
| | | | | | (RFXANK-like), 2 | | |
| | | | | | (ANKRA2), mRNA. | | |
| 1356 | 1360 | 7400246 | NM_181708.1 | NM_181708 | BCDIN3 domain | BCDIN3D | — |
| | | | | | containing (BCDIN3D), | | |
| | | | | | mRNA. | | |
| 1357 | 1361 | 7400521 | NM_006403.2 | NM_006403 | neural precursor cell | NEDD9 | dJ761I2.1; |
| | | | | | expressed, | | dJ49G10.2; CAS-L; |
| | | | | | developmentally down- | | HEF1; CASL |
| | | | | | regulated 9 (NEDD9), | | |
| | | | | | transcript variant 1, | | |
| | | | | | mRNA. | | |
| 1358 | 1362 | 7400546 | NM_021132.1 | NM_021132 | protein phosphatase 3 | PPP3CB | CALNA2; CALNB |
| | | | | | (formerly 2B), catalytic | | |
| | | | | | subunit, beta isoform | | |
| | | | | | (PPP3CB), mRNA. | | |
| 1359 | 1363 | 7510097 | NM_181270.2 | NM_181270 | CKLF-like MARVEL | CMTM1 | CKLFH; CKLFSF1; |
| | | | | | transmembrane | | CKLFH1; MGC71870 |
| | | | | | domain containing 1 | | |
| | | | | | (CMTM1), transcript | | |
| | | | | | variant 2, mRNA. | | |
| 1360 | 1364 | 7510204 | NM_016403.3 | NM_016403 | CWC15 homolog (S. cerevisiae) | CWC15 | ORF5; Cwf15; |
| | | | | | (CWC15), | | HSPC148; C11orf5 |
| | | | | | mRNA. | | |
| 1361 | 1365 | 7510332 | NM_001029991.1 | NM_001029991 | methyltransferase 11 | METT11D1 | FLJ20859 |
| | | | | | domain containing 1 | | |
| | | | | | (METT11D1), transcript | | |
| | | | | | variant 1, mRNA. | | |
| 1362 | 1366 | 7510356 | NM_001347.2 | NM_001347 | diacylglycerol kinase, | DGKQ | DAGK7; DAGK4; |
| | | | | | theta 110 kDa (DGKQ), | | DAGK |
| | | | | | mRNA. | | |
| 1363 | 1367 | 7510537 | NM_005138.1 | NM_005138 | SCO cytochrome | SCO2 | MGC125823; |
| | | | | | oxidase deficient | | MGC125825; SCO1L |
| | | | | | homolog 2 (yeast) | | |
| | | | | | (SCO2), nuclear gene | | |
| | | | | | encoding mitochondrial | | |
| | | | | | protein, mRNA. | | |
| 1364 | 1368 | 7510538 | NM_001012614.1 | NM_001012614 | C-terminal binding | CTBP1 | BARS; MGC104684 |
| | | | | | protein 1 (CTBP1), | | |
| | | | | | transcript variant 2, | | |
| | | | | | mRNA. | | |
| 1365 | 1370 | 7550341 | NM_014056.1 | NM_014056 | HIG1 domain family, | HIGD1A | HIG1; |
| | | | | | member 1A (HIGD1A), | | DKFZP564K247 |
| | | | | | mRNA. | | |
| 1366 | 1371 | 7550437 | XM_001128220.1 | XM_001128220 | PREDICTED: pleckstrin | PLEKHM1 | — |
| | | | | | homology domain | | |
| | | | | | containing, family M | | |
| | | | | | (with RUN domain) | | |
| | | | | | member 1 (PLEKHM1), | | |
| | | | | | mRNA. | | |
| 1367 | 1372 | 7550601 | NM_175932.1 | NM_175932 | proteasome (prosome, | PSMD13 | HSPC027; p40.5 |
| | | | | | macropain) 26S | | |
| | | | | | subunit, non-ATPase, | | |
| | | | | | 13 (PSMD13), | | |
| | | | | | transcript variant 2, | | |
| | | | | | mRNA. | | |
| 1368 | 1373 | 7550639 | NM_138335.1 | NM_138335 | glucosamine-6- | GNPDA2 | SB52 |
| | | | | | phosphate deaminase | | |
| | | | | | 2 (GNPDA2), mRNA. | | |
| 1369 | 1374 | 7560092 | NM_032273.2 | NM_032273 | transmembrane protein | TMEM126A | DKFZp586C1924 |
| | | | | | 126A (TMEM126A), | | |
| | | | | | mRNA. | | |
| 1370 | 1375 | 7560097 | NM_005765.2 | NM_005765 | ATPase, H+ | ATP6AP2 | ELDF10; APT6M8-9; |
| | | | | | transporting, lysosomal | | MGC99577; XMRE; |
| | | | | | accessory protein 2 | | M8-9; ATP6IP2; |
| | | | | | (ATP6AP2), mRNA. | | MSTP009; MRXE; |
| | | | | | | | HT028; ATP6M8-9 |
| 1371 | 1376 | 7560129 | NM_153028.1 | NM_153028 | zinc finger protein 75a | ZNF75A | FLJ31529 |
| | | | | | (ZNF75A), mRNA. | | |
| 1372 | 1377 | 7560180 | NM_022486.3 | NM_022486 | sushi domain | SUSD1 | RP11-4O1.1 |
| | | | | | containing 1 (SUSD1), | | |
| | | | | | mRNA. | | |
| 1373 | 1378 | 7560435 | NM_018053.2 | NM_018053 | XK, Kell blood group | XKR8 | XRG8; FLJ10307; |
| | | | | | complex subunit-related | | RP11-460I13.3 |
| | | | | | family, member 8 | | |
| | | | | | (XKR8), mRNA. | | |
| 1374 | 1379 | 7560465 | NM_001042450.1 | NM_001042450 | solute carrier family 5 | SLC5A10 | FLJ25217; SGLT5 |
| | | | | | (sodium/glucose | | |
| | | | | | cotransporter), member | | |
| | | | | | 10 (SLC5A10), | | |
| | | | | | transcript variant 2, | | |
| | | | | | mRNA. | | |
| 1375 | 1380 | 7570673 | NM_003364.2 | NM_003364 | uridinephosphorylase 1 | UPP1 | UP; UPASE; |
| | | | | | (UPP1), transcript | | UDRPASE; UPP |
| | | | | | variant 1, mRNA. | | |
| 1376 | 1381 | 7610097 | NM_006515.1 | NM_006515 | SET domain and | SETMAR | METNASE |
| | | | | | mariner transposase | | |
| | | | | | fusion gene (SETMAR), | | |
| | | | | | mRNA. | | |
| 1377 | 1382 | 7610138 | NM_004396.2 | NM_004396 | DEAD (Asp-Glu-Ala- | DDX5 | HUMP68; |
| | | | | | Asp) box polypeptide 5 | | DKFZp686J01190; |
| | | | | | (DDX5), mRNA. | | p68; HLR1; G17P1 |
| 1378 | 1383 | 7610433 | NM_005628.1 | NM_005628 | solute carrier family 1 | SLC1A5 | M7V1; ATBO; |
| | | | | | (neutral amino acid | | FLJ31068; ASCT2; |
| | | | | | transporter), member 5 | | AAAT; R16; M7VS1; |
| | | | | | (SLC1A5), mRNA. | | RDRC |
| 1379 | 1384 | 7610538 | NM_006026.2 | NM_006026 | H1 histone family, | H1FX | MGC8350; H1X; |
| | | | | | member X (H1FX), | | MGC15959 |
| | | | | | mRNA. | | |
| 1380 | 1385 | 7610563 | NM_003112.3 | NM_003112 | Sp4 transcription factor | SP4 | MGC130009; SPR-1; |
| | | | | | (SP4), mRNA. | | HF1B; MGC130008 |
| 1381 | 1386 | 7610593 | NM_001042462.1 | NM_001042462 | trafficking protein | TRAPPC5 | MGC52424 |
| | | | | | particle complex 5 | | |
| | | | | | (TRAPPC5), transcript | | |
| | | | | | variant 3, mRNA. | | |
| 1382 | 1387 | 7610754 | NM_018094.2 | NM_018094 | G1 to S phase | GSPT2 | GST2; FLJ10441; |
| | | | | | transition 2 (GSPT2), | | eRF3b |
| | | | | | mRNA. | | |
| 1383 | 1388 | 7650026 | NM_001044391.1 | NM_001044391 | mucin 1, cell surface | MUC1 | PEMT; PUM; PEM; |
| | | | | | associated (MUC1), | | EMA; H23AG; MAM6; |
| | | | | | transcript variant 6, | | CD227 |
| | | | | | mRNA. | | |
| 1384 | 1389 | 7650152 | NM_006098.4 | NM_006098 | guanine nucleotide | GNB2L1 | HLC-7; RACK1; |
| | | | | | binding protein (G | | PIG21; Gnb2-rs1; |
| | | | | | protein), beta | | H12.3 |
| | | | | | polypeptide 2-like 1 | | |
| | | | | | (GNB2L1), mRNA. | | |
| 1385 | 1390 | 7650333 | NM_001042465.1 | NM_001042465 | prosaposin (variant | PSAP | GLBA; SAP1; |
| | | | | | Gaucher disease and | | MGC110993; |
| | | | | | variant metachromatic | | FLJ00245 |
| | | | | | leukodystrophy) | | |
| | | | | | (PSAP), transcript | | |
| | | | | | variant 2, mRNA. | | |
| 1386 | 1391 | 7650433 | NM_003254.2 | NM_003254 | TIMP metallopeptidase | TIMP1 | TIMP; FLJ90373; |
| | | | | | inhibitor 1 (TIMP1), | | CLGI; EPA; EPO; HCI |
| | | | | | mRNA. | | |
| 1387 | 1392 | 10333 | XM_001129369.1 | XM_001129369 | PREDICTED: similar to | LOC731682 | — |
| | | | | | HLA class II | | |
| | | | | | histocompatibility | | |
| | | | | | antigen, DQ(1) alpha | | |
| | | | | | chain precursor (DC-4 | | |
| | | | | | alpha chain) | | |
| | | | | | (LOC731682), mRNA. | | |
| 1388 | 1393 | 60341 | XM_926322.1 | XM_926322 | PREDICTED: similar to | LOC653171 | — |
| | | | | | MAPK-interacting and | | |
| | | | | | spindle-stabilizing | | |
| | | | | | protein (LOC653171), | | |
| | | | | | mRNA. | | |
| 1389 | 1394 | 130692 | AW168583 | AW168583 | xi89f08.x1 | — | — |
| | | | | | NCI_CGAP_Mel3 | | |
| | | | | | cDNA clone | | |
| | | | | | IMAGE: 2652999 3, | | |
| | | | | | mRNA sequence | | |
| 1390 | 1395 | 150224 | NM_001010864.1 | NM_001010864 | similar to CG32542-PA | LOC196752 | FLJ34302 |
| | | | | | (LOC196752), mRNA. | | |
| 1391 | 1396 | 240392 | AK091904 | AK091904 | cDNA FLJ34585 fis, | — | — |
| | | | | | clone KIDNE2008758 | | |
| 1392 | 1397 | 270133 | NM_020362.3 | NM_020362 | chromosome 1 open | C1orf128 | TXNL1CL; HT014; |
| | | | | | reading frame 128 | | RP5-886K2.4 |
| | | | | | (C1orf128), mRNA. | | |
| 1393 | 1398 | 290687 | XM_936240.1 | XM_936240 | PREDICTED: similar to | LOC653884 | — |
| | | | | | FUS interacting protein | | |
| | | | | | (serine-arginine rich) 1 | | |
| | | | | | (LOC653884), mRNA. | | |
| 1394 | 1399 | 430519 | NM_014612.3 | NM_014612 | family with sequence | FAM120A | MGC111527; |
| | | | | | similarity 120A | | C9orf10; |
| | | | | | (FAM120A), mRNA. | | MGC133257; |
| | | | | | | | DNAPTP1; DNAPTP5 |
| 1395 | 1400 | 520403 | NM_144736.3 | NM_144736 | hypothetical protein | PRO1853 | — |
| | | | | | PRO1853 (PRO1853), | | |
| | | | | | transcript variant 1, | | |
| | | | | | mRNA. | | |
| 1396 | 1401 | 540747 | BE622355 | BE622355 | 601441142F1 | — | — |
| | | | | | NIH_MGC_72 cDNA | | |
| | | | | | clone IMAGE: 3915971 | | |
| | | | | | 5, mRNA sequence | | |
| 1397 | 1402 | 580703 | XM_001128002.1 | XM_001128002 | PREDICTED: similar to | LOC728153 | — |
| | | | | | FAM133B protein, | | |
| | | | | | transcript variant 1 | | |
| | | | | | (LOC728153), mRNA. | | |
| 1398 | 1403 | 650626 | NM_024109.2 | NM_024109 | chromosome 16 open | C16orf68 | FLJ12433; MGC2654 |
| | | | | | reading frame 68 | | |
| | | | | | (C16orf68), mRNA. | | |
| 1399 | 1404 | 670010 | XM_939387.1 | XM_939387 | PREDICTED: similar to | LOC650298 | — |
| | | | | | 40S ribosomal protein | | |
| | | | | | S26 (LOC650298), | | |
| | | | | | mRNA. | | |
| 1400 | 1405 | 730379 | XM_941195.2 | XM_941195 | PREDICTED: similar to | LOC388621 | — |
| | | | | | ribosomal protein L21 | | |
| | | | | | (LOC388621), mRNA. | | |
| 1401 | 1406 | 780079 | XM_943677.1 | XM_943677 | PREDICTED: similar to | LOC654053 | — |
| | | | | | hypothetical | | |
| | | | | | LOC389634 | | |
| | | | | | (LOC654053), mRNA. | | |
| 1402 | 1407 | 780187 | NM_145063.2 | NM_145063 | chromosome 6 open | C6orf130 | MGC19570; |
| | | | | | reading frame 130 | | dJ34B21.3 |
| | | | | | (C6orf130), mRNA. | | |
| 1403 | 1408 | 780717 | XM_925839.1 | XM_925839 | PREDICTED: | LOC158301 | — |
| | | | | | hypothetical protein | | |
| | | | | | LOC158301 | | |
| | | | | | (LOC158301), mRNA. | | |
| 1404 | 1409 | 840463 | BI827704 | BI827704 | 603074125F1 | — | — |
| | | | | | NIH_MGC_119 cDNA | | |
| | | | | | clone IMAGE: 5165863 | | |
| | | | | | 5, mRNA sequence | | |
| 1405 | 1410 | 990711 | NM_207009.2 | NM_207009 | family with sequence | FAM45A | — |
| | | | | | similarity 45, member A | | |
| | | | | | (FAM45A), mRNA. | | |
| 1406 | 1411 | 1010458 | XR_017629.1 | XR_017629 | PREDICTED: similar to | LOC645466 | — |
| | | | | | coiled-coil domain | | |
| | | | | | containing 55 | | |
| | | | | | (LOC645466), mRNA. | | |
| 1407 | 1412 | 1070470 | AK091337 | AK091337 | cDNA FLJ34018 fis, | — | — |
| | | | | | clone FCBBF2002801 | | |
| 1408 | 1413 | 1090110 | NM_001025072.2 | NM_001025072 | chromosome 3 open | C3orf17 | DKFZP434F2021 |
| | | | | | reading frame 17 | | |
| | | | | | (C3orf17), transcript | | |
| | | | | | variant 2, mRNA. | | |
| 1409 | 1414 | 1090112 | NM_058181.1 | NM_058181 | chromosome 21 open | C21orf57 | FLJ46907 |
| | | | | | reading frame 57 | | |
| | | | | | (C21orf57), transcript | | |
| | | | | | variant 1, mRNA. | | |
| 1410 | 1415 | 1110541 | NM_001009923.1 | NM_001009923 | chromosome 20 open | C20orf30 | HSPC274; |
| | | | | | reading frame 30 | | dJ1116H23.2.1 |
| | | | | | (C20orf30), transcript | | |
| | | | | | variant 1, mRNA. | | |
| 1411 | 1416 | 1230600 | XM_944489.1 | XM_944489 | PREDICTED: | LOC651064 | — |
| | | | | | hypothetical protein | | |
| | | | | | LOC651064 | | |
| | | | | | (LOC651064), mRNA. | | |
| 1412 | 1417 | 1260360 | XM_001133820.1 | XM_001133820 | PREDICTED: | LOC729776 | — |
| | | | | | hypothetical protein | | |
| | | | | | LOC729776 | | |
| | | | | | (LOC729776), mRNA. | | |
| 1413 | 1418 | 1300369 | NR_002797.1 | NR_002797 | hypothetical protein | LOC255783 | — |
| | | | | | LOC255783 | | |
| | | | | | (LOC255783) on | | |
| | | | | | chromosome 19. | | |
| 1414 | 1419 | 1340669 | BX537514 | BX537514 | mRNA; cDNA | — | — |
| | | | | | DKFZp313N0919 (from | | |
| | | | | | clone | | |
| | | | | | DKFZp313N0919) | | |
| 1415 | 1420 | 1440050 | NM_173691.2 | NM_173691 | chromosome 9 open | C9orf75 | MGC131933; RP11- |
| | | | | | reading frame 75 | | 350O14.7; FLJ90254 |
| | | | | | (C9orf75), mRNA. | | |
| 1416 | 1421 | 1450452 | NM_024331.3 | NM_024331 | chromosome 20 open | C20orf121 | DKFZp686E0870; |
| | | | | | reading frame 121 | | MGC2470 |
| | | | | | (C20orf121), transcript | | |
| | | | | | variant 1, mRNA. | | |
| 1417 | 1422 | 1690600 | NM_014661.3 | NM_014661 | family with sequence | FAM53B | RP11-12J10.2; |
| | | | | | similarity 53, member B | | KIAA0140; bA12J10.2 |
| | | | | | (FAM53B), mRNA. | | |
| 1418 | 1423 | 1740026 | XM_937227.1 | XM_937227 | PREDICTED: similar to | LOC653962 | — |
| | | | | | Teratocarcinoma- | | |
| | | | | | derived growth factor 2 | | |
| | | | | | (Epidermal growth | | |
| | | | | | factor-like cripto protein | | |
| | | | | | CR3) (Cripto-3 growth | | |
| | | | | | factor) (LOC653962), | | |
| | | | | | mRNA. | | |
| 1419 | 1424 | 1820670 | XM_935752.1 | XM_935752 | PREDICTED: similar to | LOC641978 | — |
| | | | | | general transcription | | |
| | | | | | factor II I (LOC641978), | | |
| | | | | | mRNA. | | |
| 1420 | 1425 | 1940296 | XM_932678.1 | XM_932678 | PREDICTED: similar to | LOC87841 | — |
| | | | | | ribosomal protein L13a, | | |
| | | | | | transcript variant 2 | | |
| | | | | | (LOC387841), mRNA. | | |
| 1421 | 1426 | 1940470 | XM_001132754.1 | XM_001132754 | PREDICTED: similar to | LOC728734 | — |
| | | | | | kidney-specific protein | | |
| | | | | | (KS), transcript variant | | |
| | | | | | 1 (LOC728734), | | |
| | | | | | mRNA. | | |
| 1422 | 1427 | 1980088 | BC014384 | BC014384 | Homo sapiens, clone | — | — |
| | | | | | IMAGE: 4052238, | | |
| | | | | | mRNA, partial cds | | |
| 1423 | 1428 | 1980369 | NM_001001701.1 | NM_001001701 | HCV F-transactivated | LOC401152 | — |
| | | | | | protein 1 (LOC401152), | | |
| | | | | | mRNA. | | |
| 1424 | 1429 | 2030044 | NM_015609.2 | NM_015609 | chromosome 1 open | C1orf144 | MGC70432; |
| | | | | | reading frame 144 | | DKFZp566C0424 |
| | | | | | (C1orf144), mRNA. | | |
| 1425 | 1430 | 2060014 | AK130294 | AK130294 | cDNA FLJ26784 fis, | — | — |
| | | | | | clone PRS04220 | | |
| 1426 | 1431 | 2060767 | NM_001077697.1 | NM_001077697 | similar to testis specific | LOC728137 | — |
| | | | | | protein, Y-linked 1 | | |
| | | | | | (LOC728137), mRNA. | | |
| 1427 | 1432 | 2120376 | NM_032870.1 | NM_032870 | chromosome 6 open | C6orf111 | DKFZp564B0769; |
| | | | | | reading frame 111 | | FLJ90147; SRrp130; |
| | | | | | (C6orf111), mRNA. | | MGC104269; |
| | | | | | | | bA98I9.2; RP11- |
| | | | | | | | 98I9.2; HSPC306; |
| | | | | | | | FLJ14752; FLJ14992; |
| | | | | | | | FLJ14853 |
| 1428 | 1433 | 2120468 | NM_032333.2 | NM_032333 | chromosome 10 open | C10orf58 | MGC4248 |
| | | | | | reading frame 58 | | |
| | | | | | (C10orf58), mRNA. | | |
| 1429 | 1434 | 2120746 | CD693563 | CD693563 | EST10086 human | — | — |
| | | | | | nasopharynx cDNA, | | |
| | | | | | mRNA sequence | | |
| 1430 | 1435 | 2190114 | AA722181 | AA722181 | zh20c06.s1 | — | — |
| | | | | | Soares_pineal_gland_N3HPG | | |
| | | | | | cDNA clone | | |
| | | | | | IMAGE: 412618 3, | | |
| | | | | | mRNA sequence | | |
| 1431 | 1436 | 2320408 | NM_207035.1 | NM_207035 | chromosome 1 open | C1orf63 | RP3-465N24.4; |
| | | | | | reading frame 63 | | NPD014; |
| | | | | | (C1orf63), transcript | | DJ465N24.2.1 |
| | | | | | variant 1, mRNA. | | |
| 1432 | 1437 | 2320689 | XM_928387.1 | XM_928387 | PREDICTED: similar to | LOC653610 | — |
| | | | | | Histone H2A.o (H2A/o) | | |
| | | | | | (H2A.2) (H2a-615) | | |
| | | | | | (LOC653610), mRNA. | | |
| 1433 | 1438 | 2340324 | BX648313 | BX648313 | mRNA; cDNA | — | — |
| | | | | | DKFZp686C10170 | | |
| | | | | | (from clone | | |
| | | | | | DKFZp686C10170) | | |
| 1434 | 1439 | 2340414 | BU838786 | BU838786 | AGENCOURT_821025 | — | — |
| | | | | | 9 NIH_MGC_112 | | |
| | | | | | cDNA clone | | |
| | | | | | IMAGE: 6258046 5, | | |
| | | | | | mRNA sequence | | |
| 1435 | 1440 | 2340521 | NM_152766.2 | NM_152766 | chromosome 17 open | C17orf61 | MGC40107 |
| | | | | | reading frame 61 | | |
| | | | | | (C17orf61), mRNA. | | |
| 1436 | 1441 | 2450398 | NM_001009924.1 | NM_001009924 | chromosome 20 open | C20orf30 | HSPC274; |
| | | | | | reading frame 30 | | dJ1116H23.2.1 |
| | | | | | (C20orf30), transcript | | |
| | | | | | variant 2, mRNA. | | |
| 1437 | 1442 | 2450435 | DN997246 | DN997246 | TC125227 Human | — | — |
| | | | | | breast cancer tissue, | | |
| | | | | | large insert, pCMV | | |
| | | | | | expression library | | |
| | | | | | cDNA clone TC125227 | | |
| | | | | | 5 similar to LOC399828 | | |
| | | | | | (LOC387724), mRNA | | |
| | | | | | sequence | | |
| 1438 | 1443 | 2480309 | XM_944290.1 | XM_944290 | PREDICTED: | KIAA0492 | — |
| | | | | | KIAA0492 protein | | |
| | | | | | (KIAA0492), mRNA. | | |
| 1439 | 1444 | 2510184 | NM_030797.2 | NM_030797 | family with sequence | FAM49A | FLJ11080; FLJ33961; |
| | | | | | similarity 49, member A | | DKFZP566A1524 |
| | | | | | (FAM49A), mRNA. | | |
| 1440 | 1445 | 2510403 | XM_374029 | XM_374029 | PREDICTED: | — | — |
| | | | | | hypothetical | | |
| | | | | | LOC89089 | | |
| | | | | | (LOC389089), mRNA. | | |
| 1441 | 1446 | 2630373 | AK097979 | AK097979 | cDNA FLJ40660 fis, | — | — |
| | | | | | clone THYMU2019686 | | |
| 1442 | 1447 | 2640528 | NM_001039476.1 | NM_001039476 | chromosome 16 open | C16orf35 | CGTHBA |
| | | | | | reading frame 35 | | |
| | | | | | (C16orf35), transcript | | |
| | | | | | variant 2, mRNA. | | |
| 1443 | 1448 | 2650019 | NM_016605.1 | NM_016605 | family with sequence | FAM53C | C5orf6 |
| | | | | | similarity 53, member C | | |
| | | | | | (FAM53C), mRNA. | | |
| 1444 | 1449 | 2750360 | NM_024598.2 | NM_024598 | chromosome 16 open | C16orf57 | FLJ13154 |
| | | | | | reading frame 57 | | |
| | | | | | (C16orf57), mRNA. | | |
| 1445 | 1451 | 2850630 | NM_199250.1 | NM_199250 | chromosome 19 open | C19orf48 | MGC13170 |
| | | | | | reading frame 48 | | |
| | | | | | (C19orf48), mRNA. | | |
| 1446 | 1452 | 3060661 | NM_024948.2 | NM_024948 | chromosome 10 open | C10orf97 | DERP5; MST126; |
| | | | | | reading frame 97 | | my042; FLJ13397; |
| | | | | | (C10orf97), mRNA. | | MSTP126; CARP; |
| | | | | | | | RP11-394I23.1 |
| 1447 | 1453 | 3120440 | CR596519 | CR596519 | full-length cDNA clone | — | — |
| | | | | | CS0DI056YK21 of | | |
| | | | | | Placenta Cot 25- | | |
| | | | | | normalized of (human) | | |
| 1448 | 1454 | 3120553 | BM690036 | BM690036 | UI-E-CK1-abr-b-07-0- | — | — |
| | | | | | UI.r1 UI-E-CK1 cDNA | | |
| | | | | | clone UI-E-CK1-abr-b- | | |
| | | | | | 07-0-UI5, mRNA | | |
| | | | | | sequence | | |
| 1449 | 1455 | 3130333 | XM_931058.1 | XM_931058 | PREDICTED: similar to | LOC642749 | — |
| | | | | | Succinate | | |
| | | | | | dehydrogenase | | |
| | | | | | [ubiquinone] | | |
| | | | | | flavoprotein subunit, | | |
| | | | | | mitochondrial precursor | | |
| | | | | | (Fp) (Flavoprotein | | |
| | | | | | subunit of complex II), | | |
| | | | | | transcript variant 2 | | |
| | | | | | (LOC642749), mRNA. | | |
| 1450 | 1456 | 3140280 | NM_017832.2 | NM_017832 | chromosome 9 open | C9orf6 | CG-8; FLJ20457 |
| | | | | | reading frame 6 | | |
| | | | | | (C9orf6), mRNA. | | |
| 1451 | 1457 | 3170037 | DR977977 | DR977977 | SM011178 Brain 3 EST | — | — |
| | | | | | cDNA clone ID_11178 | | |
| | | | | | 3′, mRNA sequence | | |
| 1452 | 1458 | 3180053 | NM_022833.2 | NM_022833 | family with sequence | FAM129B | OC58; MEG-3; |
| | | | | | similarity 129, member | | FLJ13518; FLJ22298; |
| | | | | | B (FAM129B), | | RP11-356B19.6; |
| | | | | | transcript variant 1, | | bA356B19.6; |
| | | | | | mRNA. | | FLJ22151; |
| | | | | | | | DKFZP434H0820; |
| | | | | | | | C9orf88 |
| 1453 | 1459 | 3180541 | NM_001013699.1 | NM_001013699 | histone H3-like | LOC440093 | — |
| | | | | | (LOC440093), mRNA. | | |
| 1454 | 1460 | 3310746 | NM_019054.2 | NM_019054 | family with sequence | FAM35A | MGC5560; |
| | | | | | similarity 35, member A | | bA163M19.1 |
| | | | | | (FAM35A), mRNA. | | |
| 1455 | 1461 | 3360170 | R25153 | R25153 | yh36h11.s1 Soares | — | — |
| | | | | | placenta Nb2HP cDNA | | |
| | | | | | clone IMAGE: 131877 | | |
| | | | | | 3, mRNA sequence | | |
| 1456 | 1462 | 3370053 | BC036926 | BC036926 | T cell receptor beta | — | — |
| | | | | | variable 21-1, mRNA | | |
| | | | | | (cDNA clone | | |
| | | | | | MGC: 46491 | | |
| | | | | | IMAGE: 5225843), | | |
| | | | | | complete cds | | |
| 1457 | 1463 | 3440670 | XM_377933.3 | XM_377933 | PREDICTED: similar to | LOC402251 | — |
| | | | | | eukaryotic translation | | |
| | | | | | elongation factor 1 | | |
| | | | | | alpha 2 (LOC402251), | | |
| | | | | | mRNA. | | |
| 1458 | 1464 | 3610372 | NM_032359.2 | NM_032359 | chromosome 3 open | C3orf26 | MGC4308 |
| | | | | | reading frame 26 | | |
| | | | | | (C3orf26), mRNA. | | |
| 1459 | 1465 | 3610682 | BX117372 | BX117372 | BX117372 | — | — |
| | | | | | NCI_CGAP_Co4 cDNA | | |
| | | | | | clone | | |
| | | | | | IMAGp998E042410, | | |
| | | | | | mRNA sequence | | |
| 1460 | 1466 | 3710682 | XM_936103.1 | XM_936103 | PREDICTED: similar to | LOC642033 | — |
| | | | | | ATP-binding cassette, | | |
| | | | | | sub-family F, member 1 | | |
| | | | | | isoform b | | |
| | | | | | (LOC642033), mRNA. | | |
| 1461 | 1467 | 3780382 | NM_138368.3 | NM_138368 | DKFZp761E198 protein | DKFZp761E198 | PP1030 |
| | | | | | (DKFZp761E198), | | |
| | | | | | mRNA. | | |
| 1462 | 1468 | 3850754 | NM_025249.1 | NM_025249 | KIAA1683 (KIAA1683), | KIAA1683 | MGC131731 |
| | | | | | mRNA. | | |
| 1463 | 1469 | 3990435 | BX282075 | BX282075 | BX282075 | — | — |
| | | | | | NIH_MGC_120 cDNA | | |
| | | | | | clone | | |
| | | | | | IMAGp998M2011561; | | |
| | | | | | IMAGE: 5223355, | | |
| | | | | | mRNA sequence | | |
| 1464 | 1470 | 4010433 | XM_939919.2 | XM_939919 | PREDICTED: similar to | LOC650832 | — |
| | | | | | mitogen-activated | | |
| | | | | | protein kinase kinase 3 | | |
| | | | | | isoform A | | |
| | | | | | (LOC650832), mRNA. | | |
| 1465 | 1471 | 4040086 | XM_936105.1 | XM_936105 | PREDICTED: | LOC642035 | — |
| | | | | | hypothetical protein | | |
| | | | | | LOC642035 | | |
| | | | | | (LOC642035), mRNA. | | |
| 1466 | 1472 | 4040300 | AK054653 | AK054653 | cDNA FLJ30091 fis, | — | — |
| | | | | | clone BNGH41000017 | | |
| 1467 | 1473 | 4060685 | XR_015313.1 | XR_015313 | PREDICTED: similar to | LOC653080 | — |
| | | | | | Beta-glucuronidase | | |
| | | | | | precursor | | |
| | | | | | (LOC653080), misc | | |
| | | | | | RNA. | | |
| 1468 | 1474 | 4120377 | NM_024520.1 | NM_024520 | chromosome 2 open | C2orf47 | FLJ22555; |
| | | | | | reading frame 47 | | DKFZp666A212 |
| | | | | | (C2orf47), mRNA. | | |
| 1469 | 1475 | 4200753 | BI024234 | BI024234 | CM3-MT0357-260101- | — | — |
| | | | | | 690-b10 MT0357 | | |
| | | | | | cDNA, mRNA | | |
| | | | | | sequence | | |
| 1470 | 1476 | 4210286 | XM_938283.1 | XM_938283 | PREDICTED: | C17orf68 | — |
| | | | | | chromosome 17 open | | |
| | | | | | reading frame 68 | | |
| | | | | | (C17orf68), mRNA. | | |
| 1471 | 1477 | 4220364 | NM_134447.1 | NM_134447 | chromosome 19 open | C19orf2 | URI; FLJ10575; RMP; |
| | | | | | reading frame 2 | | NNX3 |
| | | | | | (C19orf2), transcript | | |
| | | | | | variant 2, mRNA. | | |
| 1472 | 1478 | 4230735 | NM_052966.2 | NM_052966 | family with sequence | FAM129A | NIBAN; FLJ38228; |
| | | | | | similarity 129, member | | C1orf24 |
| | | | | | A (FAM129A), | | |
| | | | | | transcript variant 2, | | |
| | | | | | mRNA. | | |
| 1473 | 1479 | 4250192 | BX447862 | BX447862 | BX447862 T CELLS | — | — |
| | | | | | (JURKAT CELL LINE) | | |
| | | | | | cDNA clone | | |
| | | | | | CS0DH002YG04 3- | | |
| | | | | | PRIME, mRNA | | |
| | | | | | sequence | | |
| 1474 | 1480 | 4260296 | NM_003203.3 | NM_003203 | chromosome 2 open | C2orf3 | TCF9; GCF; DNABF |
| | | | | | reading frame 3 | | |
| | | | | | (C2orf3), mRNA. | | |
| 1475 | 1481 | 4290575 | NM_024315.2 | NM_024315 | chromosome 7 open | C7orf23 | MM-TRAG; |
| | | | | | reading frame 23 | | MGC4175 |
| | | | | | (C7orf23), mRNA. | | |
| 1476 | 1482 | 4390086 | AW015915 | AW015915 | UI-H-BI0-aao-g-05-0- | — | — |
| | | | | | UI.s1 | | |
| | | | | | NCI_CGAP_Sub1 | | |
| | | | | | cDNA clone | | |
| | | | | | IMAGE: 2710088 3, | | |
| | | | | | mRNA sequence | | |
| 1477 | 1483 | 4480753 | NM_199283.4 | NM_199283 | hypothetical protein | LOC220686 | — |
| | | | | | LOC220686 | | |
| | | | | | (LOC220686), mRNA. | | |
| 1478 | 1484 | 4540064 | NM_025161.3 | NM_025161 | chromosome 17 open | C17orf70 | FAAP100; FLJ30151; |
| | | | | | reading frame 70 | | FLJ22175 |
| | | | | | (C17orf70), mRNA. | | |
| 1479 | 1485 | 4540386 | XM_379543.4 | XM_379543 | PREDICTED: | LOC401442 | — |
| | | | | | hypothetical | | |
| | | | | | LOC401442 | | |
| | | | | | (LOC401442), mRNA. | | |
| 1480 | 1486 | 4570064 | NR_002186.1 | NR_002186 | hypothetical protein | DKFZp586I1420 | — |
| | | | | | DKFZp586I1420 | | |
| | | | | | (DKFZp586I1420) on | | |
| | | | | | chromosome 7. | | |
| 1481 | 1487 | 4640132 | NM_173518.2 | NM_173518 | chromosome 8 open | C8orf45 | FLJ25692 |
| | | | | | reading frame 45 | | |
| | | | | | (C8orf45), mRNA. | | |
| 1482 | 1489 | 4760537 | NM_199483.1 | NM_199483 | chromosome 20 open | C20orf24 | PNAS-11; RIP5 |
| | | | | | reading frame 24 | | |
| | | | | | (C20orf24), transcript | | |
| | | | | | variant 2, mRNA. | | |
| 1483 | 1490 | 4760601 | XM_001127981.1 | XM_001127981 | PREDICTED: similar to | LOC728014 | — |
| | | | | | huntingtin interacting | | |
| | | | | | protein 1 related | | |
| | | | | | (LOC728014), mRNA. | | |
| 1484 | 1491 | 4850161 | XM_001133058.1 | XM_001133058 | PREDICTED: | LOC728844 | — |
| | | | | | hypothetical | | |
| | | | | | LOC728844 | | |
| | | | | | (LOC728844), mRNA. | | |
| 1485 | 1492 | 4860280 | AK055969 | AK055969 | cDNA FLJ31407 fis, | — | — |
| | | | | | clone NT2NE2000137 | | |
| 1486 | 1493 | 4860309 | XM_376420.3 | XM_376420 | PREDICTED: similar to | LOC401206 | — |
| | | | | | 40S ribosomal protein | | |
| | | | | | S25 (LOC401206), | | |
| | | | | | mRNA. | | |
| 1487 | 1494 | 4860646 | NM_020223.2 | NM_020223 | family with sequence | FAM20C | RNS; DMP4 |
| | | | | | similarity 20, member C | | |
| | | | | | (FAM20C), mRNA. | | |
| 1488 | 1495 | 4880392 | NM_025182.2 | NM_025182 | KIAA1539 (KIAA1539), | KIAA1539 | P1.11659_5; |
| | | | | | mRNA. | | FLJ11560; RP11- |
| | | | | | | | 182N22.6; |
| | | | | | | | bA182N22.6 |
| 1489 | 1496 | 5130661 | NM_019018.1 | NM_019018 | family with sequence | FAM105A | FLJ11127 |
| | | | | | similarity 105, member | | |
| | | | | | A (FAM105A), mRNA. | | |
| 1490 | 1497 | 5220438 | NM_080748.1 | NM_080748 | chromosome 20 open | C20orf52 | MGC111180; |
| | | | | | reading frame 52 | | bA353C18.2 |
| | | | | | (C20orf52), mRNA. | | |
| 1491 | 1498 | 5290433 | BX105338 | BX105338 | BX105338 | — | — |
| | | | | | Soares_pregnant_uterus_NbHPU | | |
| | | | | | cDNA clone | | |
| | | | | | IMAGp998C114347, | | |
| | | | | | mRNA sequence | | |
| 1492 | 1499 | 5310181 | XM_938283.2 | XM_938283 | PREDICTED: | C17orf68 | — |
| | | | | | chromosome 17 open | | |
| | | | | | reading frame 68 | | |
| | | | | | (C17orf68), mRNA. | | |
| 1493 | 1500 | 5340162 | NM_018330.4 | NM_018330 | KIAA1598 (KIAA1598), | KIAA1598 | shootin1; shootin-1; |
| | | | | | mRNA. | | MGC40476; |
| | | | | | | | DKFZp686A0439 |
| 1494 | 1501 | 5420289 | NM_206837.1 | NM_206837 | chromosome 1 open | C1orf102 | NOR1; MGC26685; |
| | | | | | reading frame 102 | | OSCP1 |
| | | | | | (C1orf102), transcript | | |
| | | | | | variant 2, mRNA. | | |
| 1495 | 1502 | 5420349 | XM_937154.1 | XM_937154 | PREDICTED: similar to | LOC648099 | — |
| | | | | | positive cofactor 2, | | |
| | | | | | glutamine/Q-rich- | | |
| | | | | | associated protein | | |
| | | | | | isoform b | | |
| | | | | | (LOC648099), mRNA. | | |
| 1496 | 1503 | 5560279 | XM_001128421.1 | XM_001128421 | PREDICTED: | LOC728069 | — |
| | | | | | hypothetical | | |
| | | | | | LOC728069 | | |
| | | | | | (LOC728069), mRNA. | | |
| 1497 | 1504 | 5720414 | NM_017887.1 | NM_017887 | chromosome 1 open | C1orf123 | FLJ20580; RP5- |
| | | | | | reading frame 123 | | 1024G6.3 |
| | | | | | (C1orf123), mRNA. | | |
| 1498 | 1505 | 5720768 | AL049435 | AL049435 | mRNA; cDNA | — | — |
| | | | | | DKFZp586B0220 (from | | |
| | | | | | clone | | |
| | | | | | DKFZp586B0220) | | |
| 1499 | 1506 | 5810196 | NM_015395.1 | NM_015395 | DKFZP434B0335 | DKFZP434B0335 | FLJ90593; FLJ23419 |
| | | | | | protein | | |
| | | | | | (DKFZP434B0335), | | |
| | | | | | mRNA. | | |
| 1500 | 1507 | 5810632 | NM_014306.3 | NM_014306 | chromosome 22 open | C22orf28 | HSPC117; RP1- |
| | | | | | reading frame 28 | | 149A16.6; |
| | | | | | (C22orf28), mRNA. | | DJ149A16.6 |
| 1501 | 1508 | 5810671 | NM_032350.4 | NM_032350 | chromosome 7 open | C7orf50 | MGC11257; |
| | | | | | reading frame 50 | | YCR016W |
| | | | | | (C7orf50), mRNA. | | |
| 1502 | 1509 | 5820180 | AI539492 | AI539492 | te46f04.x1 | — | — |
| | | | | | Soares_NhHMPu_S1 | | |
| | | | | | cDNA clone | | |
| | | | | | IMAGE: 2089759 3, | | |
| | | | | | mRNA sequence | | |
| 1503 | 1510 | 5820504 | XM_001128859.1 | XM_001128859 | PREDICTED: similar to | LOC728944 | — |
| | | | | | THAP domain- | | |
| | | | | | containing protein 4 | | |
| | | | | | (LOC728944), mRNA. | | |
| 1504 | 1511 | 5960021 | AK023329 | AK023329 | cDNA FLJ13267 fis, | — | — |
| | | | | | clone OVARC1000964 | | |
| 1505 | 1512 | 5960167 | XR_017252.1 | XR_017252 | PREDICTED: | LOC284988 | — |
| | | | | | hypothetical | | |
| | | | | | LOC284988 | | |
| | | | | | (LOC284988), mRNA. | | |
| 1506 | 1513 | 6020433 | NM_198534.1 | NM_198534 | chromosome 19 open | C19orf45 | FLJ35784 |
| | | | | | reading frame 45 | | |
| | | | | | (C19orf45), mRNA. | | |
| 1507 | 1514 | 6100239 | NM_153706.2 | NM_153706 | chromosome 5 open | C5orf35 | MGC33648 |
| | | | | | reading frame 35 | | |
| | | | | | (C5orf35), mRNA. | | |
| 1508 | 1515 | 6110246 | NM_024627.5 | NM_024627 | chromosome 22 open | C22orf29 | FLJ21125 |
| | | | | | reading frame 29 | | |
| | | | | | (C22orf29), mRNA. | | |
| 1509 | 1516 | 6180470 | NM_001025780.1 | NM_001025780 | family with sequence | FAM108B1 | CGI-67; RP11- |
| | | | | | similarity 108, member | | 409O11.2; C9orf77 |
| | | | | | B1 (FAM108B1), | | |
| | | | | | transcript variant 2, | | |
| | | | | | mRNA. | | |
| 1510 | 1517 | 6200747 | XM_941684.2 | XM_941684 | PREDICTED: similar to | LOC220433 | — |
| | | | | | 40S ribosomal protein | | |
| | | | | | S4, X isoform | | |
| | | | | | (LOC220433), mRNA. | | |
| 1511 | 1518 | 6220762 | XM_001134398.1 | XM_001134398 | PREDICTED: | LOC730256 | — |
| | | | | | hypothetical protein | | |
| | | | | | LOC730256 | | |
| | | | | | (LOC730256), mRNA. | | |
| 1512 | 1519 | 6270112 | NM_182901.2 | NM_182901 | chromosome 11 open | C11orf17 | BCA3; AKIP1 |
| | | | | | reading frame 17 | | |
| | | | | | (C11orf17), transcript | | |
| | | | | | variant 1, mRNA. | | |
| 1513 | 1520 | 6280750 | AK128384 | AK128384 | cDNA FLJ46527 fis, | — | — |
| | | | | | clone THYMU3034853 | | |
| 1514 | 1521 | 6370082 | CR743148 | CR743148 | CR743148 | — | — |
| | | | | | NCI_CGAP_GC4 | | |
| | | | | | cDNA clone | | |
| | | | | | IMAGp971L0563; | | |
| | | | | | IMAGE: 1550800 5, | | |
| | | | | | mRNA sequence | | |
| 1515 | 1522 | 6520241 | NM_001001794.2 | NM_001001794 | family with sequence | FAM116B | MGC33692 |
| | | | | | similarity 116, member | | |
| | | | | | B (FAM116B), mRNA. | | |
| 1516 | 1523 | 6520661 | NM_018465.2 | NM_018465 | chromosome 9 open | C9orf46 | FLJ39176; FLJ14688; |
| | | | | | reading frame 46 | | AD025; MDS030 |
| | | | | | (C9orf46), mRNA. | | |
| 1517 | 1524 | 6550139 | XR_018917.1 | XR_018917 | PREDICTED: similar to | LOC441034 | — |
| | | | | | 60S ribosomal protein | | |
| | | | | | L7a (LOC441034), | | |
| | | | | | mRNA. | | |
| 1518 | 1525 | 6560072 | AW954199 | AW954199 | EST366269 MAGE | — | — |
| | | | | | resequences, MAGC | | |
| | | | | | cDNA, mRNA | | |
| | | | | | sequence | | |
| 1519 | 1526 | 6560176 | AI821401 | AI821401 | ye15f04.x5 Stratagene | — | — |
| | | | | | lung (#937210) cDNA | | |
| | | | | | clone IMAGE: 117823 3 | | |
| | | | | | similar to contains | | |
| | | | | | element MER6 | | |
| | | | | | repetitive element;, | | |
| | | | | | mRNA sequence | | |
| 1520 | 1527 | 6580129 | NM_020233.4 | NM_020233 | chromosome 17 open | C17orf48 | MDS006; NBLA03831 |
| | | | | | reading frame 48 | | |
| | | | | | (C17orf48), mRNA. | | |
| 1521 | 1528 | 6620292 | NM_022153.1 | NM_022153 | chromosome 10 open | C10orf54 | PP2135; GI24; SISP1 |
| | | | | | reading frame 54 | | |
| | | | | | (C10orf54), mRNA. | | |
| 1522 | 1529 | 6660296 | NM_058188.1 | NM_058188 | chromosome 21 open | C21orf67 | PRED54; |
| | | | | | reading frame 67 | | MGC149387; |
| | | | | | (C21orf67), mRNA. | | MGC149386 |
| 1523 | 1530 | 6660711 | NM_025138.3 | NM_025138 | chromosome 13 open | C13orf23 | FLJ23780; |
| | | | | | reading frame 23 | | bA50D16.2; RP11- |
| | | | | | (C13orf23), transcript | | 50D16.2; FLJ12661 |
| | | | | | variant 1, mRNA. | | |
| 1524 | 1531 | 6840324 | NR_002803.1 | NR_002803 | RPL13-2 pseudogene | LOC283345 | RRPL13L |
| | | | | | (LOC283345) on | | |
| | | | | | chromosome 12. | | |
| 1525 | 1532 | 6900458 | XR_015292.1 | XR_015292 | PREDICTED: similar to | LOC728481 | — |
| | | | | | similar to RPL23AP7 | | |
| | | | | | protein (LOC728481), | | |
| | | | | | mRNA. | | |
| 1526 | 1533 | 6900520 | XM_942586.1 | XM_942586 | PREDICTED: | LOC651309 | — |
| | | | | | hypothetical protein | | |
| | | | | | LOC651309 | | |
| | | | | | (LOC651309), mRNA. | | |
| 1527 | 1534 | 6940181 | NM_152644.2 | NM_152644 | family with sequence | FAM24B | MGC45962; |
| | | | | | similarity 24, member B | | DKFZp667I0323 |
| | | | | | (FAM24B), mRNA. | | |
| 1528 | 1536 | 7000274 | XM_929431.1 | XM_929431 | PREDICTED: similar to | LOC644039 | — |
| | | | | | 60S ribosomal protein | | |
| | | | | | L10 (QM protein) | | |
| | | | | | (Tumor suppressor | | |
| | | | | | QM) (Laminin receptor | | |
| | | | | | homolog) | | |
| | | | | | (LOC644039), mRNA. | | |
| 1529 | 1537 | 7050463 | DA098517 | DA098517 | DA098517 BRACE3 | — | — |
| | | | | | cDNA clone | | |
| | | | | | BRACE3009543 5, | | |
| | | | | | mRNA sequence | | |
| 1530 | 1538 | 7100632 | NM_152350.2 | NM_152350 | chromosome 17 open | C17orf45 | MGC40157; |
| | | | | | reading frame 45 | | FLJ25777 |
| | | | | | (C17orf45), mRNA. | | |
| 1531 | 1539 | 7150017 | NM_001040437.1 | NM_001040437 | chromosome 6 open | C6orf48 | G8; D6S57 |
| | | | | | reading frame 48 | | |
| | | | | | (C6orf48), transcript | | |
| | | | | | variant 1, mRNA. | | |
| 1532 | 1540 | 7210161 | BX101409 | BX101409 | BX101409 | — | — |
| | | | | | NCI_CGAP_Pr1 cDNA | | |
| | | | | | clone | | |
| | | | | | IMAGp998B182517, | | |
| | | | | | mRNA sequence | | |
| 1533 | 1541 | 7210300 | NM_173473.2 | NM_173473 | chromosome 10 open | C10orf104 | FLJ33728; |
| | | | | | reading frame 104 | | bA570G20.3 |
| | | | | | (C10orf104), mRNA. | | |
| 1534 | 1542 | 7210484 | NM_199184.1 | NM_199184 | chromosome 6 open | C6orf108 | RCL; RP3-330M21.3; |
| | | | | | reading frame 108 | | dJ330M21.3 |
| | | | | | (C6orf108), transcript | | |
| | | | | | variant 2, mRNA. | | |
| 1535 | 1543 | 7330070 | NM_174919.2 | NM_174919 | hypothetical protein | LOC201175 | — |
| | | | | | LOC201175 | | |
| | | | | | (LOC201175), mRNA. | | |
| 1536 | 1544 | 7330523 | NM_001014279.1 | NM_001014279 | chromosome 5 open | C5orf39 | AX2R; AXIIR |
| | | | | | reading frame 39 | | |
| | | | | | (C5orf39), mRNA. | | |
| 1537 | 1545 | 7380390 | NR_002773.1 | NR_002773 | AOC3 pseudogene | LOC90586 | — |
| | | | | | (LOC90586) on | | |
| | | | | | chromosome 17. | | |
| 1538 | 1546 | 7610309 | AV652851 | AV652851 | AV652851 GLC cDNA | — | — |
| | | | | | clone GLCDEG06 3, | | |
| | | | | | mRNA sequence | | |

| RNA1538 | | | | Minimal p- |
|---|---|---|---|---|
| Index | Probe Sequence | P or N Predictor or HSK gene | Minimal p-value Precision-weighted T-test | value Standard Heteroscedastic T-test |
| 1 | ATATTCCATCCTGCCCAA | N | 0.037332164 | 0.000988007 |
| | CCCTTCCTCTCCCATCCT | | | |
| | CAAAAAAGGGCCAT | | | |
| 2 | GCCCAGAGAGAGCTGTC | P | 0.001981785 | 0.00068905 |
| | CTCTCATTGGGTGAACTG | | | |
| | ATTGAGGAAGGGTCT | | | |
| 3 | ACCTAACGGTTCTCATGC | N | 0.018504517 | 0.011526675 |
| | GGTGCGTAATTGTAGATG | | | |
| | CATGTACTTGTGTG | | | |
| 4 | TCAGCACATGGAAGGCC | P | 0.032983026 | 0.002980549 |
| | CCTGGTATGGACACTGAA | | | |
| | AGGAAGGGCTGGTCC | | | |
| 5 | TCACAGTTCTGGAGGCTG | N | 0.017352387 | 0.00246003 |
| | AGAAGATCGTGAGGCTG | | | |
| | CATCTGGCAAGGGCC | | | |
| 6 | GACCCCCTTTTAAGCCAG | N | 0.041675799 | 0.020246951 |
| | TGAGCTGGGCTTCAGTTT | | | |
| | TTCCCAGGCCATGC | | | |
| 7 | GAATACTTCTCTTGCTGA | P | 0.017275273 | 0.009130841 |
| | GAGCCGATGCCCGTCCC | | | |
| | CGGGCCAGCAGGGAT | | | |
| 8 | GCAAAAGTGAAGCAGGA | P | 0.01225196 | 0.001468372 |
| | AAGAAGGGCCCTGGCAG | | | |
| | GCCAACAGGCTCAAAG | | | |
| 9 | CTCGGCTACAACATGCG | N | 0.025693928 | 5.44535E−05 |
| | GTCAAACTTGTTTCGAGG | | | |
| | GGCTGCTGAGGAGAC | | | |
| 10 | AAGCAGCTGGTGTGGCA | P | 0.040720428 | 0.002633846 |
| | GGTTCAGGAGAAGTGGC | | | |
| | ACCTGGTGGAGGACCT | | | |
| 11 | GGATTCTAGGTGGACATT | N | 0.005331291 | 0.003121417 |
| | ACAGAGTTGAATTCCTCA | | | |
| | CTACCCCCTCCCGC | | | |
| 12 | GCTGTCCCTTGGGAATG | P | 0.02710595 | 0.006979527 |
| | GGCCCTCAGAGGACAGT | | | |
| | GCTTCCAAGTACATCT | | | |
| 13 | GCTCTCTGCCTCCGGTCA | P | 0.014982241 | 0.004435676 |
| | CTCTTGCTGTGGTGCTAC | | | |
| | GTGGAAGTGAATGG | | | |
| 14 | GGACTTGTTACTAAGCAG | N | 0.021401921 | 3.61367E−05 |
| | ATTTAAGGGTCAGTGGG | | | |
| | GGAAGGCTATCAACC | | | |
| 15 | GAACCAGTAGTCCAGGG | P | 0.028514638 | 0.000900452 |
| | TGGCTCACAAAGACCACT | | | |
| | TTGAGGCTCTTGCTC | | | |
| 16 | TAAGGCCCTGCACTGAAA | P | 0.024090015 | 0.004075409 |
| | ATGCAAGCTCAGGCGCC | | | |
| | GGTGGTCGTTGTGAC | | | |
| 17 | GATAGGATTCCTTAAGAT | N | 0.027728716 | 6.12919E−05 |
| | GTTACCACCCAGGGGGC | | | |
| | CACAAGCCAGCCTGC | | | |
| 18 | GTACCGCTGCCAACACC | N | 0.002720448 | 0.001024047 |
| | CATTGACCTCCTCGTTTT | | | |
| | TGCCCGCCTTCTCCA | | | |
| 19 | CTGTGGGAGGGCTTCTT | N | 0.028462166 | 0.000297434 |
| | CCCTGTGCGCTGTTGCC | | | |
| | CATCCAAGCCTAATAT | | | |
| 20 | TCAGAGGATGAGGAGGA | P | 0.028825106 | 0.000852077 |
| | GTATGTTGTGGAGAAGGT | | | |
| | GCTAGACAGGCGCGT | | | |
| 21 | CTCCACTGGTGACAGAG | N | 0.024597969 | 8.99202E−05 |
| | AAGACACCAGGGTTTGG | | | |
| | GGGATGCCTGGGACTT | | | |
| 22 | GGGACTCAGCATTTTCCA | N | 0.021834229 | 0.000496175 |
| | GTCTTTTTCAGGGGTAGA | | | |
| | CAGGGGAGCCTGGG | | | |
| 23 | ATGTAGCAGAATGGCACC | P | 0.007622049 | 0.000468379 |
| | CAGACCACTGCCCACCA | | | |
| | GTGACGGACATGCAC | | | |
| 24 | GCAGTGATCAGGGTCCT | N | 0.035419444 | 0.000372001 |
| | GCAAGCAGTGGGGAAGG | | | |
| | GGGCCAAGGTATTGGA | | | |
| 25 | ACATGTTCCGATGCCTGT | P | 0.00391759 | 0.002067277 |
| | GGAAGACATGCCGACGT | | | |
| | CTCCTCTGCCTAGGG | | | |
| 26 | ACACAGAGGAAGTTGGC | P | 0.041093374 | 0.014034901 |
| | TAGAGGCCGGTCCCTTC | | | |
| | CTTGGGCCCCTCTCAT | | | |
| 27 | TCAGGAGGGGCCAAGAA | N | 0.046922294 | 0.000644216 |
| | CCAGGGGGCCATCAAAA | | | |
| | GCATCGGGATTTGGCA | | | |
| 28 | GAGGTGTTTGCATGTGG | P | 0.012859845 | 0.00253581 |
| | CCATTACCGTCATTGGCC | | | |
| | TGTGAAGCATTGGAC | | | |
| 29 | AGGCTCGGGGGTCCCCG | N | 0.040045541 | 0.005852028 |
| | CGTCCCAGGCCCAGGGG | | | |
| | GATGGGGGTCGCGAGA | | | |
| 30 | TACGCCTTCAGCGAGAAC | P | 0.043127654 | 0.000581297 |
| | CCTCTGCCCACAGTGGA | | | |
| | GATTGCCATCCGGAA | | | |
| 31 | GTGGTCTGTAGCCCAATA | N | 0.005178045 | 5.12006E−05 |
| | ACTGGGGAACGAGTTAC | | | |
| | AGACAAACATCACCG | | | |
| 32 | GAGTCTTCGTGGATGATG | P | 0.002407557 | 0.000800511 |
| | TGACCATTGAGGACCTGT | | | |
| | CAGGCTACATGGAG | | | |
| 33 | GTGCTGCATTGTCTGAAG | P | 0.01846435 | 0.004300257 |
| | TTAGCACCTCTTGGACTG | | | |
| | AATCGTTTGTCTAG | | | |
| 34 | CTGTTCCGATTTGCCTCT | P | 0.032766586 | 0.003191998 |
| | GAGAACGATCTCCCAGAA | | | |
| | TGGAAGGAGCGAGG | | | |
| 35 | CTAGTCCCCCCACTAGAG | N | 0.034820338 | 4.92477E−05 |
| | ACTGAGAAGTTGCCTCGC | | | |
| | AAACGAGCAGGGGC | | | |
| 36 | GAGGCTTGCTCCTATGG | P | 0.019029343 | 0.00117229 |
| | CTCCATTCCTGTGGTGGA | | | |
| | AGACGTGATGACAGC | | | |
| 37 | GATGGAGTTGACCTGGC | P | 0.03824342 | 0.001544114 |
| | AATGATCTGTGGCTAACA | | | |
| | TGCCGTCTCTCTGCC | | | |
| 38 | GAGCACCTTGTTACAGTT | P | 0.002915199 | 0.000206666 |
| | CCGGCCTCTCAGTATGTG | | | |
| | GGCTAAATGCCAGC | | | |
| 39 | CAGCCAAGAGCTGAGGG | P | 0.019880093 | 0.015887782 |
| | TAAGGGCAGGTAGGCGT | | | |
| | GAGGCTGTGGACATTT | | | |
| 40 | CACTGCAGGGCAGCGGG | N | 0.027593311 | 0.003370428 |
| | TATTCTCCTCCCCACCTA | | | |
| | AGTCTCTGGGAAGAA | | | |
| 41 | GTTTGGTCAAGGGGTAG | P | 0.011246471 | 0.005489624 |
| | GTGCAACCCAATGGACC | | | |
| | ACTTATGCAAAAGATG | | | |
| 42 | ATGGGGCACAGAGGAAG | N | 0.043753676 | 0.000552001 |
| | TTGCTGCTTGGCTGGATC | | | |
| | TGCTCAATTTGGGAG | | | |
| 43 | GTTGGTGGTGTTTGAGG | N | 0.000868885 | 0.000483754 |
| | GTTGGCTAGAAATGAAAG | | | |
| | CCTGGATTTTGTGCC | | | |
| 44 | GGCCAAGGCCATCTCCA | P | 0.049414262 | 0.000696972 |
| | AGAACGTGCTCTTCGCTC | | | |
| | ACCTGGATGACAACG | | | |
| 45 | CTGTAATTAGCTCCACGT | N | 0.041201724 | 0.000981316 |
| | GTACCCCCTTCACTCCCT | | | |
| | CCCACCAGCTCTGC | | | |
| 46 | AGTGGAGCGGCCGCCGG | P | 0.002800365 | 0.00326773 |
| | AGATGCCTGACGCATCTG | | | |
| | TCTGAGGAGCGGTCA | | | |
| 47 | CGCCTGATGTCGGGACA | P | 0.019848148 | 0.009406841 |
| | GCCCTGCTCCCAAGTACA | | | |
| | AATAGAGTGACCCGT | | | |
| 48 | CCAGATGGCATGGTTGCT | N | 0.000707601 | 0.000469973 |
| | CTATTGGACTACCGTGAG | | | |
| | GATGGTGTGACCCC | | | |
| 49 | GCACCCAGCGGAATGTG | HSK | HSK | HSK |
| | CTTAGTATTTGGTCACCA | | | |
| | GCCGTCATCCTGGGC | | | |
| 50 | TCCAGCAGTGGTCATTCG | N | 0.009966308 | 0.001088538 |
| | ACAACGAAAGTCATACCG | | | |
| | TAGAAAAGATGGCG | | | |
| 51 | CAGACCCAGAGAAAAGTA | N | 0.013671386 | 0.002749976 |
| | GTTGTCAGTCATAGCACA | | | |
| | CATCGGACATTTGG | | | |
| 52 | GCAATGGTAAACCTCGAG | N | 0.001814082 | 9.08413E−05 |
| | ACAACAAACAAGCAGGG | | | |
| | GTGTTTGAACCAACC | | | |
| 53 | GTGAGCCTGGGCCCTAC | P | 0.009398616 | 0.000918585 |
| | ATGGATGTGGTCGTCTCC | | | |
| | CTGGTCACTATCATG | | | |
| 54 | CAGAGGCAGAGGATGCT | N | 0.001627582 | 0.000849497 |
| | GCAAGAGAGAAAAGCTG | | | |
| | CAAAAGAGGCCGCCGC | | | |
| 55 | GTTGGGGAAGAGGATAA | N | 0.009360761 | 5.94033E−05 |
| | GGTTATATCTAGGACAAC | | | |
| | TCTTTGAGTTGGTCC | | | |
| 56 | CTGAGAAGGAACTGGCT | N | 0.001468154 | 0.000118685 |
| | GCTGAAAAGAAACGCATC | | | |
| | CTGCACTGCCTGGGG | | | |
| 57 | CGAAGTCAGAAAACTCAT | P | 0.006818317 | 0.000590803 |
| | CATCAGGCGACGCCCTG | | | |
| | GCGGCTGGGTGGAGA | | | |
| 58 | GGGCAAACCCAAAGATG | N | 0.019614515 | 1.54054E−06 |
| | GAAAGTGCTTGTTGGGTG | | | |
| | GGTAAGCACCACCTG | | | |
| 59 | ATGTACGTGGGGGATTCT | N | 0.012771344 | 1.7475E−05 |
| | TGACTCGGGTTAGTCTCT | | | |
| | GGGGATGCAGAGCC | | | |
| 60 | TTTCGTTTGAGTCCTGCT | P | 0.012870801 | 0.007058607 |
| | GTTGGTGTCGGAGCACG | | | |
| | AGGGGAGGCACGGTG | | | |
| 61 | GGACCCTGTTGCTAAGC | P | 0.004036969 | 0.000411885 |
| | CCCAGCAAGCAATCCTAG | | | |
| | GTAGGGTTTAATCCC | | | |
| 62 | CAGTGTGGTGAAGGTTG | N | 0.004098879 | 0.001708665 |
| | ACTGAAGAAGTCCAGTGT | | | |
| | GTCCAGTTAAAACAG | | | |
| 63 | CAACTTTCAGAGCCTCTT | P | 0.007348034 | 0.000538906 |
| | GTATTTGGAAGGCTGGAA | | | |
| | GGGCCCAGACTTTG | | | |
| 64 | ACACAGTAGCGATGGAG | P | 0.008240878 | 0.000130326 |
| | GTGACGTAGCTTCCTCCG | | | |
| | AGTGGAACTGCAGCC | | | |
| 65 | CCCTCCCTGTGGAGCCT | P | 0.010600382 | 0.003694658 |
| | GTTACCTCCGCATTTGAC | | | |
| | ACGAGTCTGCTGTGA | | | |
| 66 | GTTTGGTGTGTTCCCGCA | P | 0.030046219 | 0.000791838 |
| | AACCCCCTTTGTGCTGTG | | | |
| | GGGCTGGTAGCTCA | | | |
| 67 | AGCCATAGCTGGTGACAA | N | 0.015966527 | 0.008984203 |
| | ACAGATGGTTGCTCAGG | | | |
| | GACAAGGTGCCTTCC | | | |
| 68 | GCGCCTTTCTCATCAGCT | N | 0.008279154 | 0.005651563 |
| | TCTTCCGAGGGTGACAG | | | |
| | GTGAAAGACCCCTAC | | | |
| 69 | CATGGAGGGCAAAGAGC | N | 0.018391268 | 0.000887428 |
| | TCAGCAAAGGGCAAGCC | | | |
| | AAGAAGCTGAAGAAGC | | | |
| 70 | GCATTGGGGCCAAACAC | P | 0.029415319 | 0.015178021 |
| | AGAATCAGCAAAGAGGA | | | |
| | GGCCATGCGCTGGTTC | | | |
| 71 | GCATCAGACTTTTAATCT | P | 0.021705071 | 0.001460624 |
| | GAGGGTCCAGGGTTCAA | | | |
| | GTCCCTGTTCGGGCG | | | |
| 72 | GGGGAGAGGAAAAGTGG | N | 0.024140153 | 0.020459406 |
| | ATGGAAGTGTCTGGAAAG | | | |
| | GGCACGAGAGAGTCT | | | |
| 73 | TGCCAGAACACAAGACAC | N | 0.033188881 | 0.006635147 |
| | CAAATTGAACTCACTGCT | | | |
| | TTTGAGGCATCTGG | | | |
| 74 | AGCATCTTTCATATGGTA | N | 0.014552301 | 0.00674199 |
| | GGAACCAACAAGGAAACT | | | |
| | TTCCTTTAACTCCC | | | |
| 75 | TACCGCCTCCTCCCCGTC | N | 0.041006567 | 3.6805E−05 |
| | GCTCTGCCTTTTCCAAAA | | | |
| | CTCACTTGGGCCCT | | | |
| 76 | GAACCCGCGTGCAACCT | P | 0.015932773 | 0.019756877 |
| | GTCCCGACTCTAGCCGC | | | |
| | CTCTTCAGCACGCCAT | | | |
| 77 | CCCCTTGGGGAAGACGA | N | 0.004848855 | 0.00204549 |
| | AGGGATGCTGCAGTTCC | | | |
| | AAAAGAGAAGGACTCT | | | |
| 78 | CCTCTGCTCCTCCCTTCC | N | 0.002769245 | 0.001149087 |
| | CAAGGCATTGAAGCTGAA | | | |
| | TGTGCCAACTGGCA | | | |
| 79 | GCTTGTGGGTCATCTTGC | P | 0.005689907 | 3.51486E−05 |
| | ACCTTTACAAACAAGGAA | | | |
| | TTCCCCTCTGTGCC | | | |
| 80 | CTTTTTTGTACGTAGCTG | N | 0.009261833 | 0.002535233 |
| | TTACATGTAGGGCAATCT | | | |
| | GTCTTTAAGTAGGG | | | |
| 81 | CCCACCATCACCTCAAAC | N | 0.003288948 | 0.003275589 |
| | CCAATCACCCCCTCCTCT | | | |
| | GTATGCTGTCACAC | | | |
| 82 | GGGGGCACTATAGCCAC | N | 0.008604899 | 0.000681996 |
| | TAAACGAGGTGTGAAAG | | | |
| | GCTCAAGAGGATGACC | | | |
| 83 | AAAGTACTGCGCGACAAT | P | 0.011343988 | 0.00097227 |
| | ATCCAGGGCATCACCAA | | | |
| | GCCGGCCATCCGGCG | | | |
| 84 | GGGGAAGCCCGGGGCC | N | 0.031645706 | 0.000409337 |
| | GCCCGGGACCTCGGCCC | | | |
| | GTTCCTCCGGACCCGAG | | | |
| 85 | CCGGGCTCCTAGCGGGG | N | 0.026199581 | 7.30733E−05 |
| | AAAAGGAAGGGGATAACT | | | |
| | CAGAGGAACAGACAC | | | |
| 86 | GAGCAAAACTGCACAAAC | N | 0.00257002 | 0.000470189 |
| | TTGCACATTGGAAAGTGC | | | |
| | AACAAGTTCCCGTG | | | |
| 87 | CGCCACTTCATGGAGCT | P | 0.007681502 | 0.003121849 |
| | GGTGACTTGTGGCCTTTC | | | |
| | CAAAAACCCATATCT | | | |
| 88 | GGTGGCTGCGCGAGGGA | N | 0.000207886 | 0.000291014 |
| | CCGAGTACTAGAGCTGCT | | | |
| | TGCATGCGTTACTAA | | | |
| 89 | CTGCACTGCGTGCTGGT | P | 0.028714119 | 0.013807898 |
| | GACGAATCCACATTCATC | | | |
| | TCAATGGAAGGATCC | | | |
| 90 | AAGGACTCTTCCACCAGA | N | 0.008984509 | 0.002853643 |
| | GATGGGAAAACCACTGG | | | |
| | GGAGGACTAGGACCC | | | |
| 91 | GTGGGTCTCACCTCTCCA | N | 0.031525302 | 0.000801824 |
| | TTGTTCTCTTGTTCTATG | | | |
| | GGGCAGGTTTGGGG | | | |
| 92 | ACCAAGGGAGAACCAGG | P | 0.005085163 | 0.000658284 |
| | AAACGGAAACAGAGTGG | | | |
| | TCATTCCCCAGCCCGG | | | |
| 93 | GGGGCATCTGGCATGGA | N | 0.024159522 | 0.000179822 |
| | CTGGGGTGGAAATGGGG | | | |
| | ATGTCAGTTTGAAAGC | | | |
| 94 | GCCTGAGGTGACAGACA | P | 0.027191026 | 0.001043909 |
| | GGGCAGGTGGTAACAAA | | | |
| | ACCGTTGAACCTCCCA | | | |
| 95 | CATGGCCAAACGTACCAA | N | 0.020811279 | 0.012763214 |
| | GAAAGTCGGGATCGTCG | | | |
| | GTAAATACGGGACCC | | | |
| 96 | GCTCCGTGTTGGAAAAAA | N | 0.001352071 | 0.000494316 |
| | GGGGTAGTGCATTTTAAA | | | |
| | TTGACCTTCATACG | | | |
| 97 | GGGGTCTGTGAGAGTAC | N | 0.002473879 | 0.003326958 |
| | ATGTATTATATACAAGCA | | | |
| | CAACAGGGCTTGCAC | | | |
| 98 | GGCACAGGCTCTGCCGT | P | 0.007554333 | 6.88269E−05 |
| | GTCCTTGGAGTGAAAGAC | | | |
| | TCTTTTTACCAGAGG | | | |
| 99 | CCTGTTCCCTTCATTGCT | N | 0.047157776 | 0.008599342 |
| | GTGAGTTGGGAGTGCATT | | | |
| | GAGAGATGATGTCC | | | |
| 100 | GACCGGAAGCAACCCCT | N | 0.002980627 | 0.004409495 |
| | TCACAGACACGAGCACAT | | | |
| | CGGCAAACCCTATGA | | | |
| 101 | CCACTTCTGAGGAATGGA | N | 0.007523301 | 0.000712361 |
| | CCTGGTGTAACACACTTG | | | |
| | AATATGTGTGATGC | | | |
| 102 | CCAGCTCTACCAGCCCCT | P | 0.00400794 | 0.00250033 |
| | CAAGGATCGAGAAGATG | | | |
| | ACCAGTACAGCCACC | | | |
| 103 | CGGGCTGGCCCACCTCG | P | 0.005823665 | 0.003599139 |
| | TTTTGCTAGTGAAGAGAG | | | |
| | GCGAGAAATTGCTGA | | | |
| 104 | AGTAAGGGATCGAAGAC | N | 0.015966542 | 0.000690126 |
| | ATTTCAAATTGCTATCTCC | | | |
| | ATCTGGGCTGATCC | | | |
| 105 | GGCTGCAAGCTGGATAC | P | 0.031574076 | 0.00482945 |
| | ATGGAATTCAGCACACTT | | | |
| | TTCTCCCTCTTACTG | | | |
| 106 | GGCATTTACGTTTCTCTG | N | 0.006538796 | 0.002363376 |
| | ATGCTCCCTTGAAGCCAT | | | |
| | AGAATTTAGGGGCT | | | |
| 107 | GCAGCCCTAGAAAGTAA | N | 0.020468039 | 6.36589E−05 |
| | GCCCAGGGCTTCAGATC | | | |
| | TAAGTTAGTCCAAAAG | | | |
| 108 | TGGGAGCCCCATTGCCT | P | 0.0465622 | 0.002572124 |
| | CTGTCTCCTTCGGTGCCT | | | |
| | GCAGAGACTTTGTCT | | | |
| 109 | AACTGGTTGTGGGGAGG | N | 0.024268598 | 2.92395E−05 |
| | GAAGAGAAGGACAGGGT | | | |
| | GTTGGGGGGATGAGGA | | | |
| 110 | TACAGAACCATCCACTTG | N | 0.003269445 | 0.009058255 |
| | ACCTAACTACCTCCCCTG | | | |
| | GCCGCGCTCTCGCT | | | |
| 111 | TCCAAGGCCTGGATGCTA | N | 0.002557283 | 0.000924193 |
| | ATCAAGATGAACAGGTCG | | | |
| | ACTTTCAAGAATTC | | | |
| 112 | CCAGGGGAGGTGGGTAG | N | 0.02074243 | 4.5543E−05 |
| | AGCCCGAGGCCCCCCAG | | | |
| | TAGCCGACCCTGGCGT | | | |
| 113 | CCCGGGAGTGGATTCTA | P | 0.019704852 | 0.006056356 |
| | AATGTGATTTTCCTAGGC | | | |
| | TACTGCAGGAGCCCC | | | |
| 114 | GAGCACTCAACCCAGAA | P | 0.002545834 | 0.002083034 |
| | GGCGAAGATAGCTTTTGG | | | |
| | TTGTAGGCGGCTTCC | | | |
| 115 | GTCACCGAAAAGTGCTG | P | 0.029826357 | 0.001671524 |
| | CGGGATAACATCCAAGG | | | |
| | CATCACCAAACCGGCC | | | |
| 116 | CGACTCTCAAGGCACTGT | P | 0.038275024 | 0.012053236 |
| | GTATGCCCTGCAAGTTGG | | | |
| | CTGTCTATGAGCAT | | | |
| 117 | GCTGTCTAGGTCCGTCC | N | 0.006981971 | 0.007950141 |
| | GGTGTGTCAGATTTTCCT | | | |
| | CAGATTAGATGTGCC | | | |
| 118 | GATCTCACTGACCCGTTG | N | 0.010236169 | 0.007717381 |
| | CCCTGTAACCACTTTCTT | | | |
| | TCCTTCTTTTGCCT | | | |
| 119 | GTTTTGGTTGTGAATCAT | N | 0.030427324 | 0.000332984 |
| | TTGCCAGCGAGCCAAGG | | | |
| | GAGAGGCAGGGATTC | | | |
| 120 | AGGGAAGTGGGATCCGA | N | 0.01803385 | 0.000812944 |
| | GCCTGTAGAAGGGAGGC | | | |
| | ATGAAACTTGTGGAGG | | | |
| 121 | TTACGTTATCTACCAGAG | N | 0.0214931 | 0.008021978 |
| | CACCGTGGGCTGTTACTT | | | |
| | GCCTTGAGTTGGAA | | | |
| 122 | GGGCTGAAAACTGCCCTT | P | 0.004239413 | 0.002122611 |
| | GGGCTGACTTTTGATAGG | | | |
| | CCATGCCTTGCCAC | | | |
| 123 | GCACAGCGTCCTGTCCA | N | 0.004334836 | 0.00529578 |
| | CACCCAGCTCAGCATTTC | | | |
| | CACACCAAGCAGCAA | | | |
| 124 | GTAACCCTCCAGTGGTG | P | 0.019628615 | 0.001216422 |
| | GAAGGCACACCATGGCT | | | |
| | TCCTCTGCTTGGTTTG | | | |
| 125 | GTTGAGGGAGTCAGCAC | P | 0.022339333 | 0.002482027 |
| | AGTCCTTTCTGCAGCTTC | | | |
| | TAACCCAGGACCATG | | | |
| 126 | GTGTCCCTGGAGCAGTG | N | 0.014752098 | 0.001847445 |
| | AGGGGACACCAGCAAAA | | | |
| | ACCTTCAGCTCTCAGA | | | |
| 127 | GCCTTCGGTTGTAAGTAG | N | 0.038065763 | 0.032809685 |
| | CCAGATCCCTCTCCAGTG | | | |
| | ACATTGGAACATGC | | | |
| 128 | CCAGACTGTGATGACTG | N | 0.009421378 | 0.006557969 |
| | GGAGCGGGCTGAATGAG | | | |
| | ATGGAGTGTGCATTAC | | | |
| 129 | GCCCTCTCTGTGGATCCC | N | 0.009864615 | 0.015626713 |
| | TACTGCTGGTTTCTGCCT | | | |
| | TCTCCATGCTGAGA | | | |
| 130 | GCCTCAGGAAAACAAGA | P | 0.048658712 | 0.001081202 |
| | CCTCTGTGCACCTCACTT | | | |
| | TTGGCTCACTGCAGC | | | |
| 131 | TGTAAGACGAACTTGGAT | N | 0.029901567 | 0.000120075 |
| | CACGGCTTGGTTCAGCA | | | |
| | GAGCATGGGGGCGGG | | | |
| 132 | AACCCAGGGCTTTAGAAG | N | 0.008491795 | 8.92123E−06 |
| | GCTGAGGCTGGGGGATT | | | |
| | GCTTGAAGTCAGGAG | | | |
| 133 | TCACTTGGGAGGGACGC | N | 0.001572086 | 0.00042732 |
| | ATAGAAGGAGCTCTAGGA | | | |
| | ACACAGTGCCAGTGC | | | |
| 134 | CAAAGGGGCATCGGAGA | P | 0.011706711 | 0.003529377 |
| | AGTGCAGCTGCTGTGCC | | | |
| | TGATGTGGGAACAGCT | | | |
| 135 | ACCTGAAGCAGCAAGTG | P | 0.039208835 | 0.021102604 |
| | AGCGGGCTGGAGGGTGT | | | |
| | GCAGGACGACCTGTTC | | | |
| 136 | TCTCAAACCCGGTATGGT | N | 0.045388658 | 0.022274481 |
| | GGTCACCTTTGCTCCAGT | | | |
| | CAACGTTACAACGG | | | |
| 137 | GTCACGACATCCGAACTG | N | 0.015344888 | 0.000886132 |
| | GAGGGACAAGGATCTTAA | | | |
| | ACCCAAAGTACGAG | | | |
| 138 | GCTGCTAAAGTTCCAGCA | N | 0.046979583 | 0.014494505 |
| | AAAAAGATCACCGCCGC | | | |
| | GAGTAAAAAGGCTCC | | | |
| 139 | TAGCTGCTACCCTGGAAC | P | 0.020581476 | 0.003669674 |
| | GGTGGGCAGAGAGCCTA | | | |
| | CTAGGAAATGTGCAG | | | |
| 140 | GAAGAGTAACAAGAGTAG | N | 0.002215247 | 0.000437489 |
| | ACTGGACCAGAAATCGG | | | |
| | AGGGTGGCAAGCAGC | | | |
| 141 | CGAGAGTGCCCTTCTGAT | P | 0.00404049 | 0.000434785 |
| | GAATGTGGTGCTGGGGT | | | |
| | GTTTATGGCAAGTCA | | | |
| 142 | GGTGCTGATCCTACCACC | P | 0.002534122 | 0.001605959 |
| | TACTGCTACCTTCCTTAG | | | |
| | CTTCACCCTGGCTA | | | |
| 143 | ACCCTACTCTTCGGCCCC | P | 0.018495536 | 0.00116788 |
| | GCCAGCTCTCCATCTCAC | | | |
| | ACTTTAAGAGCCTC | | | |
| 144 | CGTAACCAAAGAACGACA | N | 0.042699055 | 0.003989645 |
| | CAGAGAGATCAACAAGCA | | | |
| | AGCCACCCGAGGGG | | | |
| 145 | GAAGTAGCCCCAGTGAG | P | 0.008798324 | 0.000770132 |
| | TGTTAGTGATGCAGCTCT | | | |
| | CCTGGCCCCAGAGGA | | | |
| 146 | GTGAGTGGTCTCTGTCG | N | 0.012969873 | 4.57892E−05 |
| | GGAAAGATGTAGGGATT | | | |
| | GGTTCTCCAGGATCTT | | | |
| 147 | CACAGCCTTACTAGTTCC | N | 0.007897073 | 0.007786129 |
| | TTGCTTCCAGTATTTCAAT | | | |
| | TGGTCTCCTCCCC | | | |
| 148 | GGCGGCCTCATCGTTCTT | P | 0.008495878 | 0.01270945 |
| | TGCCTTCCTGGTCACCAT | | | |
| | CTGCTACGCTGGAA | | | |
| 149 | CGTGGCACCCCAAAAGG | P | 0.037138165 | 0.008694684 |
| | CCTCTGCTGGCATTTGCC | | | |
| | TGTGATGACAAAGAC | | | |
| 150 | GGCTTTTCAATTCTGTGG | N | 0.042964366 | 0.027099992 |
| | ACTTTGTACCATTTGGCT | | | |
| | TCACCTTGTACTGC | | | |
| 151 | AAAGCAGATATTTCCCGG | N | 0.000457197 | 0.001746366 |
| | ACCCAGCGCGGCCTCAA | | | |
| | CCAGGGCAGGAAAGA | | | |
| 152 | ACGGCGTGGAGGACTTT | P | 0.012030947 | 0.001874106 |
| | TCCGTGAGCCAGACGAT | | | |
| | GCTGGAGGAGGTATTC | | | |
| 153 | GGGGCTACATTTGTTCAT | N | 0.03089471 | 0.024754256 |
| | TTCCAGCAGTAGCATAAA | | | |
| | CTTACGGTGACATG | | | |
| 154 | GTCCTCACGTTCCCAGGA | P | 0.016355746 | 0.00699423 |
| | GGGCGGCTTCACCCTTC | | | |
| | GTAACCAGGAGACAA | | | |
| 155 | CTGAACTGCTAATGTGGC | N | 0.031398144 | 0.004785948 |
| | TGCTTTGTAGGGAATGGA | | | |
| | CTAATATCAGTGTG | | | |
| 156 | TGCCAAGATACATTGACA | P | 0.021675389 | 0.012145095 |
| | CTGAACATGGAGGCAGC | | | |
| | CAGGCCCGTTTCCTC | | | |
| 157 | CTTGCGGAAAATGAGAAT | P | 0.034571633 | 0.015217814 |
| | TGATGGTGTCCCCAATGC | | | |
| | CCCACCTCACAGAG | | | |
| 158 | TGCATGGGGGTACCCCA | N | 0.001617601 | 0.000135135 |
| | ATCTGAAGTCAGTAAATG | | | |
| | AACTAATCTACAAGC | | | |
| 159 | AACAGCATCCTCTTCCAC | P | 0.038694398 | 0.001478528 |
| | GCTCAGAAGTGTTCTGGT | | | |
| | TGGGGCCAGGCATG | | | |
| 160 | CAGATCTACTGGCGAGC | N | 0.025042639 | 0.004060175 |
| | GATGAAAATGTTGCAGGG | | | |
| | AGAGTCAGCAGAGGC | | | |
| 161 | CCGTGGGCTGTGCCAAG | P | 0.017697722 | 0.011077006 |
| | TGTGCCCAGGGATGTGTT | | | |
| | TGCAAAGGGACATCT | | | |
| 162 | GGGATTCTGTGACTGGAA | N | 0.021955263 | 0.002057477 |
| | AAGGTGACAAGTTGGTGA | | | |
| | CTTTGACACTGCAG | | | |
| 163 | TCCTGGCCATGAGGACA | N | 0.009159399 | 0.00356599 |
| | AAAATTACTGAGTGGCCC | | | |
| | TTAAAGAGGGAAGTT | | | |
| 164 | TAATCCCGTTATGGACTC | P | 0.025232086 | 0.044454089 |
| | TGTCTCCAGGAGAGGGG | | | |
| | TCTATCCACCCCTGC | | | |
| 165 | TGTTGGCAGCGACACCAT | P | 0.019065912 | 0.001334151 |
| | CCCATACAGGCTCTTACC | | | |
| | TCTTCTCCTGAGGG | | | |
| 166 | GGCAGTTGTCTGCATTAA | N | 0.010000982 | 0.000112406 |
| | CCTGTTCATACACCCATT | | | |
| | TTGTCCCTTTATTG | | | |
| 167 | AGCTTTCTGCACCCCCAG | P | 0.040871279 | 0.00259531 |
| | TGGCATCTCCTCATCACG | | | |
| | TTCTGTGCCGTCCT | | | |
| 168 | GGGGCTTTCGTGTCCCC | N | 0.01451577 | 0.000334594 |
| | CTGTGCGGTCAGTGTTTT | | | |
| | CAGTACCACCTCTCT | | | |
| 169 | GAGCAGGTGAAGCCATC | N | 0.011876698 | 0.003317407 |
| | AAAGAATGGGGTGACCA | | | |
| | CGTGACCAACTTGTGC | | | |
| 170 | AGCCCTGATGATTGGCC | N | 0.044672319 | 0.003062544 |
| | CCACCTCCTGCTGCCCC | | | |
| | ATAACCCTCTCTTCAT | | | |
| 171 | AAAGAAAGCTGGGCCTG | N | 0.026121174 | 0.02635688 |
| | TCGAAGGATGACAGGGA | | | |
| | TGTGCTGCCAGGTTGC | | | |
| 172 | TCACCCGCACTGAGTCAA | N | 0.032826967 | 8.36096E−06 |
| | CAGACTGAGCGCGTCCA | | | |
| | GGCCTGACAGCTCTG | | | |
| 173 | GGTGTGACTTGCCTTATT | N | 0.027345905 | 0.008191302 |
| | GAACTGATACTGGCATAT | | | |
| | CTGACTGTAAGCAG | | | |
| 174 | CAAGCCTCACTTTTCTGT | N | 0.008936589 | 5.36789E−06 |
| | GCCTTCCTGAGGGGGTT | | | |
| | GGGCCGGGGAGGAAA | | | |
| 175 | TGAGGACAGTTCAGAGG | P | 0.013435976 | 0.004081858 |
| | AGGATTCAGACGAGTGTC | | | |
| | GCTGCGTGAGTGGCC | | | |
| 176 | GCTCTCTCCCATCCAAGT | N | 0.004926943 | 0.003000054 |
| | GACCAGATGCCCTACTCA | | | |
| | GCTTCCATCACCCC | | | |
| 177 | CAGCTGGTTTCCTGGGTA | P | 0.008471018 | 0.002078946 |
| | TGCCTGGACTGTTGCCCA | | | |
| | GTGTAAGATCTGTG | | | |
| 178 | AGGAGGTACAGACGGTG | P | 0.018423111 | 0.01347762 |
| | GAGGATGGGGTGTTTGA | | | |
| | CATCCACTTGTAATAG | | | |
| 179 | GGGGAACACACCTGAAA | N | 0.030934418 | 0.001904134 |
| | CTAGAGGAACAGCTTATG | | | |
| | TGGTCTATGAGGACA | | | |
| 180 | CGGACAGTGATGGCTCTT | N | 0.000473071 | 0.000865777 |
| | GGAAATGGGTGGATGGC | | | |
| | ACAGACTATAGGCAC | | | |
| 181 | ACCGTGGTGAACCCTTG | P | 0.040642634 | 0.005937424 |
| | GGGGGAGGTTCTAGCCA | | | |
| | AAGCTGGCACAGAAGA | | | |
| 182 | GGGAGTGGTGGAGCCAG | P | 0.025216093 | 0.015139534 |
| | TCGCTGTAACACTGAGCC | | | |
| | TCAGAGACGAACCAA | | | |
| 183 | AGGCCTTAAGCTTTGGAC | N | 0.004215992 | 0.004511756 |
| | CCAAGGGAAAACTGCAT | | | |
| | GGAGACGCATTTCGG | | | |
| 184 | ACTGCTGCGTCATTACAG | P | 0.002144657 | 0.000492593 |
| | GGCACAGGCCATGGATG | | | |
| | GAAAACGCTCTCTGC | | | |
| 185 | TCTGGAAGGGGACAGTG | N | 0.045579794 | 2.53683E−05 |
| | AAAAGAGGAGTGACAGG | | | |
| | AGGGAAAGGGGGAGAC | | | |
| 186 | CCTGGTCAAGTGCTGGC | N | 0.017755838 | 0.012465827 |
| | TCTGCTGTCCTTGCCTTC | | | |
| | CATTTCCCCTCTGCA | | | |
| 187 | TAGGTGTGGTGGCGTTAT | P | 0.02974907 | 0.004606371 |
| | GGCAGCCCGGCTGCTGC | | | |
| | TTGGATGCGAGCTTG | | | |
| 188 | CCTGCAGTGTAAGTACAG | N | 0.005883304 | 0.000609634 |
| | CACACTGTCAAATTCTTTT | | | |
| | CCTTAAGGTGCAC | | | |
| 189 | GGGCTTTTCCAAAAGCAA | N | 0.014467705 | 0.001401659 |
| | ACAAAGATAGGTTCCTCA | | | |
| | GGTGACCAAAACTG | | | |
| 190 | ACATCTTTCTGGCACATA | N | 0.007054921 | 0.00123624 |
| | ACTGTCTCCTTAACCACT | | | |
| | GGAACAGTTCAGCC | | | |
| 191 | GGCATTAGAGATCCAGCA | N | 0.013353268 | 0.002650261 |
| | CATTCTCAGTACTGTGGT | | | |
| | GCAGTATTAGCCCA | | | |
| 192 | CGCCCGAATCTGGCTCG | N | 0.013845835 | 0.002135094 |
| | GCGGAATACCTCTTAGAC | | | |
| | AAGCACACCCTGGGG | | | |
| 193 | GAACTTGGAGAGCATCA | N | 0.048169021 | 0.008185891 |
| | GGAAGGCCCAGCTGAAA | | | |
| | TCAGAGAATCTGCTCG | | | |
| 194 | TCCTCCTGAGCCTACTGC | P | 0.02387538 | 0.004032458 |
| | CAAACGTCCTCAGTGTTG | | | |
| | TCTGCACCTGCTCC | | | |
| 195 | CAGCTTCCAGTGGTGGC | N | 0.011484257 | 0.003844816 |
| | CGTAGACTTGGCTCGGA | | | |
| | ACTTAGTGGCACCAGA | | | |
| 196 | TCCCATGTTTTTACCCTG | N | 0.000263971 | 0.000431055 |
| | CCCCTGCCTTGATTAGAC | | | |
| | TCCTAGCACCTGGC | | | |
| 197 | GTTATGCTTGTATTGAAT | N | 0.024560935 | 0.005563563 |
| | GCTGTCTTGACATCTCTT | | | |
| | GCCTTGTCCTCCGG | | | |
| 198 | CCCGGCCAACATCAAGT | N | 0.013638772 | 0.003569124 |
| | GACTTTATAGCTGCAAGA | | | |
| | AATGTGGTATGTGGA | | | |
| 199 | CTGCTGCGACTGATGCC | P | 0.010739358 | 0.00575891 |
| | AGGACAACCTTTCTCCCA | | | |
| | GATGTAAACAGAGAG | | | |
| 200 | CCTGCATAACAACACTGG | N | 0.006611943 | 0.000644228 |
| | GCCTTCTTAACTAAAATG | | | |
| | CTCACCACTTAGCC | | | |
| 201 | GATGGACTGTGCCCGGG | N | 0.02211032 | 0.000181223 |
| | TTCTGGTCATGGACAAGG | | | |
| | GGCAGGTGGCAGAGA | | | |
| 202 | GCTGTGCAAAGGTTGAG | N | 0.012329516 | 0.008165579 |
| | AGCTATTGCTGATTAGTT | | | |
| | ACCACAGTTCTGATG | | | |
| 203 | CAGGCACCTGGCTGAGT | P | 0.011277699 | 0.000604528 |
| | GTGCTGGAGTGAGGATC | | | |
| | TTGAACAGAAACTTCC | | | |
| 204 | CAGTTATGGAGGACTTGT | N | 0.022569474 | 0.000211085 |
| | ATGGAGAAATTTAAGTCT | | | |
| | TCACTGAGGGCCAC | | | |
| 205 | TCCCCACTATAACAGTTG | N | 0.034684181 | 0.009181751 |
| | CTGCCGCCGGAAGTACA | | | |
| | GACCAGAAGCCCCTG | | | |
| 206 | TGTCCTGGCTTCCCCTCC | P | 0.041505042 | 0.013570867 |
| | CAAGGAGGATGAGGATG | | | |
| | GTGCCTCTGAGGAAA | | | |
| 207 | CCACCTCGAGAACCAAG | N | 0.013801155 | 0.002948417 |
| | GATACTTTCGGAAGAGGA | | | |
| | GCAGGAAATGTTCAG | | | |
| 208 | CTTTGTTCCTGGGGAATT | P | 0.012359302 | 0.000188549 |
| | CACTTCTCTTCCTCCCTC | | | |
| | ATGGAAGATGCAAG | | | |
| 209 | GCCATATTGGAGTAGCGA | N | 0.031803428 | 0.001028175 |
| | GGAATCTGATTCCAAGCA | | | |
| | AAAACCAGACAATG | | | |
| 210 | GCTTCAGGCGGTAAACC | N | 0.012877468 | 0.004994319 |
| | AACAGCTCACAAAGGAGA | | | |
| | AAGAGCACTACCAGG | | | |
| 211 | GAAACTGTTGAAGCTGCA | N | 0.021786768 | 0.010584883 |
| | GAACCAACGAGGTGGCC | | | |
| | GAATCCTTCTTCAGG | | | |
| 212 | GAGGGAGAAGAATAAAG | N | 0.003393734 | 0.000978005 |
| | CAGCTGCCTGGAGCCTA | | | |
| | TTCACTATGTTTATTG | | | |
| 213 | CAGGTCCTGCAGTCTGG | P | 0.042554194 | 0.024262837 |
| | CTGAGCCCTGCTTGGTTG | | | |
| | TCTCCACACACAGCT | | | |
| 214 | GGTGTCCATCAGTAACTA | P | 0.032171221 | 0.038592347 |
| | CCCCCTTTCTGCTGCCCT | | | |
| | CACCTGTGCAAAAC | | | |
| 215 | GATCCAGCCATTACTAAC | N | 0.005093561 | 5.34878E−05 |
| | CTATTCCTTTTTTGGGGA | | | |
| | AATCTGAGCCTAGC | | | |
| 216 | GGGCGGCATTTACACTGT | N | 0.017674003 | 0.00014022 |
| | GCAAGTATTGAGAAGAGT | | | |
| | GCATAAAGACAGGG | | | |
| 217 | CATCTCTGTGGCAGCGG | P | 0.015266094 | 0.009136299 |
| | CAGCTATTTACATGGCCT | | | |
| | CACAGGCATCAGCTG | | | |
| 218 | CGTGTGCCACTTGCCCA | P | 0.042064073 | 0.009794698 |
| | GCTTCTTGGGCACACAGA | | | |
| | GTTCTTCAATCCAAG | | | |
| 219 | TCTCCCAAATAAGATGTG | N | 0.015764064 | 4.09289E−06 |
| | CTGCTTACCGAGGTATCA | | | |
| | CGGGGTGGGGCTCC | | | |
| 220 | AGGGACTTTGTTTAGGCC | N | 0.003345758 | 0.000680059 |
| | AAGGAAGGAGCGGAAGT | | | |
| | AGGGCAACTCGGTCC | | | |
| 221 | CCTGCTAAGTCCGCTCCT | P | 0.02397531 | 0.000352678 |
| | GCTCCAAAAAAGGGCTC | | | |
| | CAAAAAGGCGGTGAC | | | |
| 222 | CAGCTCATGCCCTCAATG | N | 0.047966686 | 1.33729E−05 |
| | TTTATATTGTGTTATCTGT | | | |
| | TGGGTCTGGGACA | | | |
| 223 | TTGGGAGCTGAAGAATAC | N | 0.01200117 | 2.03857E−06 |
| | TGGACGGGGCTTCGGAG | | | |
| | AGGAAGGATGGTCCA | | | |
| 224 | ATTGCTCCCCAGACTGAA | P | 0.011265572 | 0.002310578 |
| | CAGAAACCTGGCCGCCG | | | |
| | GATGGGACCTCCTTT | | | |
| 225 | CCAAGGTGTTAAGGGGA | N | 0.000573704 | 0.001609945 |
| | TAGTACCTCCCAATTCAA | | | |
| | GCAGAGAAACTGACC | | | |
| 226 | GGGGCACATGTTGTAAG | N | 0.045998083 | 0.000180076 |
| | AAACTGATTGGAAGGGG | | | |
| | AAATGTGCAGCTCTCC | | | |
| 227 | ACTGCTGGCAGCGGCTT | N | 0.041263637 | 0.002590193 |
| | TCTGTATTCTGCCACACC | | | |
| | AGGGGCAGATGTTTG | | | |
| 228 | CACCCCAAGCAGTACGC | N | 0.007430483 | 0.001722221 |
| | TTGCTGGTCTAAGTCTTA | | | |
| | ACCCCAGGACTCAGA | | | |
| 229 | CTTTTCCAAGTTCCCAAG | P | 0.041075201 | 0.015325065 |
| | GCCTACAGCTGAAGCCC | | | |
| | TTAGGTACCTGTGTT | | | |
| 230 | CATACAGTAATCATGCTG | N | 0.017806575 | 0.004063292 |
| | CAGAAATTTGCAGTCTGC | | | |
| | ACCTTATGGATCAC | | | |
| 231 | CCTCTTGAGCTGGAACG | P | 0.003336886 | 0.004296026 |
| | CCTGAAACTGGAGCCTCA | | | |
| | CGAAGGGCTGCTGCT | | | |
| 232 | AGTGATTGCCTGGGCCA | N | 0.035584334 | 0.000195993 |
| | AGTGGCAGGTTGGGGAG | | | |
| | GATGGCTGCAAAGAAG | | | |
| 233 | CTTCCAGTCTTTTTAGAA | N | 0.006780813 | 0.000304009 |
| | CGTGGTGGAGGAGGGTT | | | |
| | GTGTGTGCCCCAGGG | | | |
| 234 | GGGGGGTAGAATTTAGTA | N | 0.010087694 | 0.003144485 |
| | AATATTCCAGCCGGTCGT | | | |
| | TTTATGCACAAGGC | | | |
| 235 | TGATCCGAAGGAGGAGT | P | 0.034853878 | 0.004034999 |
| | GGCGCTGGGCGCTGGAC | | | |
| | TCGCTGGTGTGAAAAT | | | |
| 236 | GAGTCTCAAGTCCGTATG | N | 0.000755288 | 0.002991841 |
| | TAAATCAGATCTCCCCTC | | | |
| | TCACCCCTCCCACC | | | |
| 237 | CAGATTTGGCACCTACTC | P | 0.004675112 | 0.001487566 |
| | CTGCCCCACAGAGCACA | | | |
| | CCACGAAACACTGTC | | | |
| 238 | GAGCTTCCCGAGAATGG | N | 0.001176973 | 0.000286601 |
| | GGCCTGGGTTTGATTCAT | | | |
| | CTGTTTTCTACAGGG | | | |
| 239 | GAGACCAGTAGATTTTCA | N | 0.041732557 | 0.000243943 |
| | ATGGGAAATGTACCTAGC | | | |
| | AAGCTGGTTCTTGC | | | |
| 240 | AACCAGGGGCCATGAAT | N | 0.034865906 | 0.000448909 |
| | CACCTTTTGGTCTGGAGG | | | |
| | GAAGCCTTGGGGCTG | | | |
| 241 | GTCCCTGTCCCTCCCAAA | P | 0.028728838 | 0.003585929 |
| | GCACAGAGCACAGAAAT | | | |
| | GAGGCCGTTTACATG | | | |
| 242 | GCAGATAGAGTGTTACCG | P | 0.005896187 | 0.000645202 |
| | ACGGGTGGAAAAGCTAC | | | |
| | GGAATCGCCAGGATG | | | |
| 243 | CAGCCATAGGTGCAGTTT | N | 0.007314375 | 0.006682156 |
| | GCTTCTACATGATGCTAA | | | |
| | AGGCTGCGAATGGG | | | |
| 244 | CCCTGGTATTGATTTCTC | P | 0.00779219 | 0.003889254 |
| | AGGACTTTGGAGGGCTC | | | |
| | TGACACCATGCTCAC | | | |
| 245 | GGCTGGCAGTCTTTGTC | N | 0.034813175 | 0.000623826 |
| | GTTGTTCATTCTGGGGAT | | | |
| | AAAGGGGAACTAGGC | | | |
| 246 | CCAGAGCCTGTGATGCC | P | 0.007285195 | 0.001073167 |
| | TCCTCAGCAGGTAGAGC | | | |
| | AGATGGAAATACCACC | | | |
| 247 | CCTGTGTTTGCATCCTCT | N | 0.005121314 | 0.008656748 |
| | GTTCCTATTCTGCCCTTG | | | |
| | CTCTGTGTCATCTC | | | |
| 248 | GGGTGCCTTCCTTGGTCA | N | 0.004561912 | 0.002998685 |
| | CCAAGGCAGTGCGTGCA | | | |
| | CGTTAGGGTTTCCTT | | | |
| 249 | CTCAAGGTCATGCAGTTA | N | 0.020310045 | 0.00517795 |
| | GTAAGTGGCAGAACAGG | | | |
| | GACTTGAACCAAGCC | | | |
| 250 | CCTCCACGTGATTCCTAC | P | 0.003557776 | 0.001323842 |
| | AGCAGTTCAAGCCGCGG | | | |
| | AGCACCAAGAGGTGG | | | |
| 251 | TGCTGCTCCTGCTGCCC | P | 0.013625029 | 0.010856486 |
| | CATGAGCTGTGCCAAGT | | | |
| | GTGCCCAGGGCTGCAT | | | |
| 252 | CTATTAACGCTACGATGC | P | 0.015462351 | 0.004802328 |
| | CTGAACCTACCAAGTCTG | | | |
| | CTCCTGCCCCAAAG | | | |
| 253 | GATGGTTCTGATGCTGTC | N | 0.017558861 | 9.16221E−06 |
| | AGCCTCTGGGTGCAAATT | | | |
| | CTGAGGGCCCGGGA | | | |
| 254 | CCCTCACGCACCCGCTC | N | 0.009263328 | 0.002613281 |
| | ACGCACCCTCGGTGAAT | | | |
| | CCTTGGTGATGATTTT | | | |
| 255 | AGTACCACTCCAAAGGCA | N | 0.007405038 | 0.001662479 |
| | AGGAACCATGATTGACAA | | | |
| | CAGTCAAGCTGTGG | | | |
| 256 | TGGAAGCCCTCACCAAG | P | 0.027769471 | 0.002064433 |
| | CACTTCCAGGACTGACCA | | | |
| | GAGGCCGCGCGTCCA | | | |
| 257 | CCCCTACTTATTGCCACA | N | 0.023488622 | 8.37796E−06 |
| | GAGGAGGGATCTTTTCCA | | | |
| | TAACTGAAGGGGAG | | | |
| 258 | GCACGACGATGAGGTGA | N | 0.004534932 | 0.000150875 |
| | CAGTCACGGCCCTGGCC | | | |
| | AACGTCAACATTGGGA | | | |
| 259 | GCACAGTTGAGGAGCCA | N | 0.014228146 | 0.002627577 |
| | GAGACTTCTTAAATCATC | | | |
| | CTTAGAACCGTGACC | | | |
| 260 | CTTTAATTCTTGGGCCTC | P | 0.001270219 | 0.001745243 |
| | CAATAAGTGTCCCATAGG | | | |
| | TGTCTGGCCAGGCC | | | |
| 261 | TACCTGGCTACAGAAAGA | N | 0.011286563 | 0.003864364 |
| | AGATGCCAGATGACACTT | | | |
| | AAGACCTACTTGTG | | | |
| 262 | CCCCTGCAAGGGTAGAG | P | 0.033071121 | 0.00057651 |
| | TCAGGTGAGAGTCCCTTG | | | |
| | GTGAGTCATTTGTAC | | | |
| 263 | GCCCAGTACTGGAGAAA | N | 0.014943906 | 5.33033E−05 |
| | ATGAAACTGGGATTGACC | | | |
| | CATCAAGATGCTTGG | | | |
| 264 | GGGACGAGACAGGTGCT | N | 0.005603143 | 0.000530127 |
| | AAAGTTGAACGAGCTGAT | | | |
| | GGATATGAACCACCA | | | |
| 265 | CCCCAGTGTGTATAAGCT | N | 0.012426241 | 0.00697227 |
| | GGCATTTCGCCAGCTTGT | | | |
| | ACGTAGCTTGCCAC | | | |
| 266 | CTGTCCCGCTGCGTGTTT | P | 0.003191835 | 0.005765734 |
| | TCCTCTTGATCGGGAACT | | | |
| | CCTGCTTCTCCTTG | | | |
| 267 | GACTCTGATGTTGGGTAG | P | 0.009787241 | 0.002360685 |
| | CTGGCCTCTGTGGGGAT | | | |
| | TGTAAGTGCCCTGAG | | | |
| 268 | CACACTGGGGCTGCCTTT | N | 0.001532941 | 0.00031371 |
| | CTCTGACTCTGTCTTCCC | | | |
| | CAAGTCAGGGGGCT | | | |
| 269 | TACCACTGCAAAGTGATG | N | 0.002811244 | 0.001328222 |
| | GAAAAGGGTGGAGAACA | | | |
| | GGGGAGTAGCCAGGC | | | |
| 270 | GCTTTCTTAGGGAAATGA | N | 0.018120486 | 0.001641551 |
| | CAGGGCAAAGCAATTTTT | | | |
| | CTGTTGGCTTTGGG | | | |
| 271 | CTACGCCATGGGACATCT | N | 0.008082882 | 0.010790438 |
| | AATTCAGAGGAAGAAGGT | | | |
| | CCATGTCTTTGGGG | | | |
| 272 | GAGGATCATTACAGAGAC | N | 0.013942164 | 0.000256697 |
| | AGACTCTCCCGAGACATG | | | |
| | GGCCACACTGATAG | | | |
| 273 | GTAACTGTAAGTTCACAT | N | 0.008931886 | 0.000713525 |
| | CAACCTCATGGGTTTGGC | | | |
| | TTGAGGCTGGTAGC | | | |
| 274 | CCTGGCCAAGTGAGGAA | N | 0.024085697 | 0.000229349 |
| | GGAAAGCAGAAAGGTGA | | | |
| | CGATTCTCACTCACCT | | | |
| 275 | CAGCCTGACGAGCTGCC | N | 0.010260699 | 0.015508533 |
| | CGAGGTGGATAACCTGA | | | |
| | CACTGGACGGGAATCC | | | |
| 276 | GCTCTGCACCATCCCTCA | P | 0.041028466 | 0.01213045 |
| | CCCAGACCGTAGACACC | | | |
| | AGGGAACCACATCTA | | | |
| 277 | CTGCGAGTTTTCGGGTG | P | 0.02703624 | 0.011983097 |
| | GGCAGACGCACTGTTGA | | | |
| | ATCTGGTAGCCAGGGT | | | |
| 278 | GAGCGTGATGATTGGGT | P | 0.000644717 | 0.001456141 |
| | GTTCATACGCTTGTGTGA | | | |
| | GATGTGCCACCCTTG | | | |
| 279 | GAAACATTCTAGTAGCCT | P | 0.001123725 | 0.000461324 |
| | GGAGAAGTTGACCTACCT | | | |
| | GTGGAGATGCCTGC | | | |
| 280 | AGGTCTCCTCTGGGAGG | P | 0.031257106 | 0.004239175 |
| | TCTTGGCCGACTCAGGG | | | |
| | ACCTAAGCCACGTTAA | | | |
| 281 | CAGGCTCATAGCAGCTAC | N | 0.00800258 | 0.004520618 |
| | TGTGTAGAAAATTCCCCC | | | |
| | TACTTCTAATTTGC | | | |
| 282 | TCTTGCTGACAGAATAGG | N | 0.009210967 | 0.003011462 |
| | TTCCGTTCTGGGCGGTG | | | |
| | GTTCTCGAGCCTGCC | | | |
| 283 | CCTCAGCAGCTGGTAATC | N | 0.006995851 | 0.013738783 |
| | TTGCTCTGCTTGACAACA | | | |
| | TCTGAGTGCAGCCG | | | |
| 284 | CATTCTGGGACTACCGTG | N | 0.003676314 | 0.001577243 |
| | AAGCCTGGAGTAGGGAG | | | |
| | AGCGAGTTTGGGAGC | | | |
| 285 | GCTGGCGTGCCCATGTT | N | 0.00154184 | 0.00204779 |
| | GCAGATATTTTCCCGAGT | | | |
| | TCCCCAGAATGGATG | | | |
| 286 | GACCTCCAGAGTGAAGAT | N | 0.001336323 | 8.76894E−06 |
| | GGGTGACTAGATGATATG | | | |
| | TGTGGGTGGGGCCG | | | |
| 287 | GGGAACTGGCATTACTG | N | 0.024336742 | 0.000719741 |
| | GAACTAATGGTTTTAACC | | | |
| | TCCTTAACCACCAGC | | | |
| 288 | CTGGGCAGTGAAGTGGA | N | 0.026178082 | 0.007555627 |
| | TATCACTGAAGGAGATAG | | | |
| | GAAGCCAGACTACAC | | | |
| 289 | AACAGCTCTGTGTGTGAA | N | 0.000598839 | 0.000670094 |
| | GGTGAGGACTCTTGGAA | | | |
| | GCAGGCCATCCTGGC | | | |
| 290 | CCATCTCCGGGACGTTCT | P | 0.046365858 | 0.032237507 |
| | CGGCTCTGCCTCATTGTG | | | |
| | TGCAGAAACTGTGG | | | |
| 291 | GGAGGCCAGACGTTGAC | N | 0.030089652 | 0.000127324 |
| | GCTGCAGGGAGAGGGTG | | | |
| | GTGGGCGCAGCCGCTA | | | |
| 292 | GGTGGGGATTCTGGAAC | N | 0.015350104 | 0.000204025 |
| | AATCATCTTAGGGGGTGT | | | |
| | GCCATGCTGTTCCTG | | | |
| 293 | AGCTTCCCAAGCTGTATG | N | 0.01447795 | 0.009585449 |
| | TGAAGCTACCTTACTGTG | | | |
| | TGAGTTGTGCAATT | | | |
| 294 | ACTGCTGCTTCCTACCTG | N | 0.003323749 | 0.000879694 |
| | CAAGACGAACAATGTATG | | | |
| | TTTCAAGGGTGAGC | | | |
| 295 | GCGCCTCCAGGCCAAGA | N | 0.024023674 | 0.002229257 |
| | AGGAGGAGTTCATCAAGA | | | |
| | CTTTATCCAAGGAGG | | | |
| 296 | GTGGAAAGGATGGGGTG | N | 0.006424061 | 6.9002E−05 |
| | GAATACAGTTGTGGGCTA | | | |
| | TTGGTAAGGTCCCAG | | | |
| 297 | TATTGCAGCCATCCATCT | N | 0.001884377 | 0.000122824 |
| | TGGGGGCTCATCCATCA | | | |
| | CACCCGGGTTGCTAG | | | |
| 298 | CGGCCCCTGAGCAAGAC | N | 0.002720947 | 0.004041925 |
| | AGTACGCTTCAACGTGCT | | | |
| | CAAGGTCACCAAGGC | | | |
| 299 | CCCAGCTGAACCCGAGG | N | 0.019928696 | 0.004179299 |
| | CTAAAGAAGATGAGGCAA | | | |
| | GAGAAAATGTACCCC | | | |
| 300 | TAGCAGCTTGGGCACCT | P | 0.013709838 | 0.001927862 |
| | CCACTCTGTGCGGTCTGA | | | |
| | TGGCCCCAGCAAGGT | | | |
| 301 | GACCGCTATGCTCAGGA | P | 0.037005567 | 0.005895429 |
| | CATGGGAGACAACTGCAT | | | |
| | TACTCAGTGATCAAG | | | |
| 302 | TGGAGGTGGTTTTGGTG | N | 0.045541181 | 0.006932617 |
| | GGAATGACAACTTTGGTC | | | |
| | ATGGAGGAAACTTCA | | | |
| 303 | ATGTGGACTGCCCTACAT | P | 0.020334873 | 0.002737801 |
| | TTGGCCTGTGCCACTGG | | | |
| | CCAACCGGAAATGGT | | | |
| 304 | TTTTGTTAACGTCTGCCA | N | 0.016459127 | 0.002365733 |
| | CCCCCACTCTCACCCCCA | | | |
| | AGCTCTAAGCCCCC | | | |
| 305 | ACTCTGGCCCCTATGGC | P | 0.00526104 | 0.000484951 |
| | GGTGGAGGCCAGTACTT | | | |
| | TGCAAAACCACGAAAC | | | |
| 306 | TCTTCCATACATTAGTTC | P | 0.018923704 | 0.008321766 |
| | CCACCATCGCATGCCCA | | | |
| | GGGACCACTGCCTGG | | | |
| 307 | AAGCTAAGGCCGCGTTG | N | 0.007036877 | 0.005840286 |
| | GGGTAAGGCCCTCACTT | | | |
| | CATCCTGCGACTAGCA | | | |
| 308 | CGACCGGCTCGTATTCC | N | 0.032215411 | 0.013902091 |
| | GATCAGTCGCTTCCATTG | | | |
| | TTAGCATCGTACACG | | | |
| 309 | AGATGTGTTTTCAGAGCT | N | 0.002118103 | 0.001911586 |
| | AGGTACAGAGGAATGTTT | | | |
| | GCTACCTTTAGCGG | | | |
| 310 | CGGGTGCAAGCCCGTGT | P | 0.006614579 | 0.002452292 |
| | GTCTGGCCTCTTTCCTCG | | | |
| | TGAAGACGATGTGTC | | | |
| 311 | CAGTGGCTACCACCTGTA | P | 0.017094823 | 0.013578455 |
| | ATCTCAGCAGTTTGGGAG | | | |
| | ACCAAAGCAGGACG | | | |
| 312 | CAAGCAAAATTGTGGGCA | N | 0.0257826 | 0.003475715 |
| | AGAGAATCCGCGTGAAA | | | |
| | CTAGATGGCAGCCGG | | | |
| 313 | GAGGGCACCAGGCACAA | N | 0.022069707 | 0.000134452 |
| | CGACATCGAGCTCTACAG | | | |
| | CCAGTACCTGGAGGG | | | |
| 314 | TCTGCAAAGGGGCGTGC | P | 0.007970418 | 0.026713487 |
| | AGCTGCTGTGTCTGATGT | | | |
| | GGGGACAGCTCTTCT | | | |
| 315 | GTGTAAGGGTCCAGCTG | N | 0.021320803 | 0.001836127 |
| | ATCAAGAATGGCAAGAAA | | | |
| | ATCACAGCCTTTGTA | | | |
| 316 | GATGGGCACCTGGATAA | N | 0.016791854 | 4.55339E−07 |
| | CTCAGGATGGGGGCTGC | | | |
| | TCACAAAGACCACATC | | | |
| 317 | GGAGCCCCTTGGAGTAT | P | 0.038277546 | 0.009228911 |
| | GGCTTTTCACATGGGCTT | | | |
| | CTATACCGCTTCGAC | | | |
| 318 | ACTGTCAGGCCAGTGCT | P | 0.020498041 | 0.003102954 |
| | GCTGCGGATGTGAGAAA | | | |
| | CCGGTGATCCGAAGGC | | | |
| 319 | CCCCTGGGCTATCATCTG | P | 0.007543595 | 0.000284438 |
| | CATGGGGCTGGGGTCCT | | | |
| | CCTGTGCTATTTGTA | | | |
| 320 | CACAAGAGTGGTCATAAG | N | 0.014947525 | 0.000279691 |
| | GGGGTTTGAACTGAGTC | | | |
| | CCACTACCTCGGGGG | | | |
| 321 | ACCCCTGGGCTACCATCT | P | 0.007981192 | 0.001868627 |
| | GCATGGGGCTGGGGTCC | | | |
| | TCCTGTGCTATTTGT | | | |
| 322 | CCCTCTCAAGTAATGGCT | N | 0.010546341 | 0.000670775 |
| | CAGCTAATAAAGGCGCAC | | | |
| | ATGACTCCCAAAAA | | | |
| 323 | GTACTTCGGGGCTCTACA | N | 0.009446875 | 0.000317343 |
| | GACAATCTGATGGATGAC | | | |
| | ATAGAAAGGGCAGT | | | |
| 324 | TTCTCAGGAATCGGCGG | N | 0.037168162 | 0.000822981 |
| | GAAGAAGCCCCCTTGAT | | | |
| | GGAGTCTGGTGGGGTT | | | |
| 325 | TGGTATTTGGGCAGCTG | P | 0.002392575 | 0.002545243 |
| | GTGATCGTTGGTCCCGG | | | |
| | CGCCCTTTCTTTACTG | | | |
| 326 | CTGTATGCCCAGGGAAA | N | 0.003513109 | 0.000731921 |
| | GTGGCGTTATAACAGGAA | | | |
| | GCAGAGTGGCTATGG | | | |
| 327 | CCAACGACTAACCCTGAA | N | 0.004462779 | 8.13533E−05 |
| | ATGGGGGTGTTCCAGCC | | | |
| | TTCAGCGAGATGGCC | | | |
| 328 | CAAGAGTGCCACAGATAT | N | 0.043049954 | 0.000963711 |
| | TCTCCTGGGGGAGGATG | | | |
| | CTGGTGTTGGGAGGG | | | |
| 329 | GAACAATGGTCGTGCCAA | N | 0.020953125 | 0.017419716 |
| | AAAGGGCCGCGGCCACA | | | |
| | TGCAGCCTATTCGCT | | | |
| 330 | CGGCCTGATGGAGAGAA | N | 0.008627467 | 0.002771479 |
| | GGAACATGTTCGACTGG | | | |
| | CTCCTGATTACAATGC | | | |
| 331 | ACTCTGTAAGGAAGTTCC | N | 0.016211841 | 0.007996184 |
| | CAAATACAAACTTATAAC | | | |
| | CCCAGCTGTGGTCT | | | |
| 332 | GTAACAGGGTGCAGTGTT | P | 0.008098257 | 0.001860315 |
| | GTTTATACTTCATTGCTC | | | |
| | CTTCAGGACATGGG | | | |
| 333 | TATGTCCTCTGATTGGGA | P | 0.003643475 | 0.000477632 |
| | CAAGGCACCTGCATTCAC | | | |
| | AGGCGGCCCTGAGC | | | |
| 334 | GGCTTGGCCACCCTGCC | N | 0.029600192 | 0.007199955 |
| | GCTGCCCAGCCACATCC | | | |
| | CTTGGTTTTGTATTTT | | | |
| 335 | TCATGGCCGCCCTCAGA | N | 0.005791868 | 0.000671519 |
| | CCCCTTGTGAAGCCCAA | | | |
| | GATCGTCAAAAAGAGA | | | |
| 336 | ATCCTCTGAGAAAACAGC | P | 0.00398372 | 0.000227825 |
| | CCACAGGACTGGGTCCT | | | |
| | CCTTATCCGTCTTGC | | | |
| 337 | AAATGACAAAGAGCGAGT | N | 0.028468123 | 0.01386108 |
| | GGCAGCTGCAATGGAAA | | | |
| | ACCCCAACTTACGGG | | | |
| 338 | AGCTCAGCGGTTACTTCG | P | 0.001345488 | 0.000396197 |
| | CGTGTCATCAAACCACCT | | | |
| | CTCTGGGTTGTTCG | | | |
| 339 | TAGAACTATTATTGACCA | P | 0.009457684 | 0.006368964 |
| | CGCCTCCTCCAAGTCCCA | | | |
| | GCGAGCCCGTGTAC | | | |
| 340 | GCACCTGCTGTAGACAG | N | 0.007478515 | 0.001297099 |
| | AAGACAGTATTCTGCAAT | | | |
| | GACTGAGAATGCAGT | | | |
| 341 | ACTGCTGCTTCCTACCTG | N | 0.002690936 | 0.002238076 |
| | CAAGACGCACAATGTATG | | | |
| | TTTCAAGGGTGAGC | | | |
| 342 | GTGCCCCTCTGTATCTTT | N | 0.015388346 | 0.006233688 |
| | TGAGAAGTGCGGAATAG | | | |
| | GTTGCTTCTACCACC | | | |
| 343 | TTGGGAGGCAGAGGCCG | N | 0.013481429 | 0.000857851 |
| | GTGGGTTGCTTTAGCTCA | | | |
| | GGAGTTGGAGACAAG | | | |
| 344 | CATATATTGCATGGAGGT | N | 0.039958262 | 0.031385449 |
| | ACCCCAATCTGAAGTCAG | | | |
| | TAAATGAACTAATC | | | |
| 345 | GTGTTTACATGTCTGTCC | P | 0.016704913 | 0.002709995 |
| | CCCCAGACTGTGAGCTC | | | |
| | CTTGAGGGCAGGGAC | | | |
| 346 | CCGTGTGCATCAGGTCCT | N | 0.037372342 | 0.022300797 |
| | TCACGGACTCCTCGGGG | | | |
| | GCCAATATTTATTTG | | | |
| 347 | GCCAGGACAGCCCTCCC | N | 0.024120571 | 0.002995754 |
| | AGCCATGAATCCTTACTC | | | |
| | AGCTACCTCGGGTTG | | | |
| 348 | CAGCAGAGAGGCCTGTG | N | 0.02458944 | 0.004630386 |
| | ACATGGGGCCCTGTACTA | | | |
| | CTGCCTGGTTCCACA | | | |
| 349 | AGAATTTCTTCACCTGAA | N | 0.043898711 | 0.043547486 |
| | TAAACCATGTGGTCAGCA | | | |
| | TTGCATCTGAGGCA | | | |
| 350 | TGTGATGACCACTACAGC | N | 0.03757579 | 0.007549704 |
| | AGAGTAAAGCATGTCCAA | | | |
| | GGAAGGATGTGCTG | | | |
| 351 | GAGGAGAAAAAGCGGTA | N | 0.0048264 | 0.009320246 |
| | CGATGCCTTCCTGACCTC | | | |
| | ACCGGCCTCCCCAAG | | | |
| 352 | AACGACACCAGCCAAAC | P | 0.028775692 | 0.004899306 |
| | CAGCAGCCCCTCAGCAT | | | |
| | CCAGCAACATAAGCGG | | | |
| 353 | AGTCTGTAGCCTCCCCGA | N | 0.023949921 | 0.022611684 |
| | TCCAAGTTCCTAGACCTC | | | |
| | ATGGCTGTCCCCTC | | | |
| 354 | TCCGCGCATCCACTTGTT | P | 0.024422559 | 0.021380902 |
| | GCAGTCCAAGTCCTCTAG | | | |
| | TGCAACGCCATAGC | | | |
| 355 | GGACTGGGAAAATCTGC | P | 0.046003869 | 0.017945159 |
| | AGCATCAGACTATGCCTT | | | |
| | TCATCCCCCAGCCAG | | | |
| 356 | AGTAATTGGCAGTGACTA | P | 0.044612313 | 0.022540253 |
| | TGGGCGCACTGCCTAAC | | | |
| | ATTTAGCCCTGCCCC | | | |
| 357 | GAGACAAAGATGGCTGC | P | 0.013299668 | 0.009092238 |
| | GAGAGTCGGCGCCTTCC | | | |
| | TCAAGAATGCCTGGGA | | | |
| 358 | TGCGTTGGTCCAGAGCG | P | 0.027915124 | 0.044845297 |
| | GAGGCTGTGTGCCTGGG | | | |
| | GGAGTTTTCCTCTATA | | | |
| 359 | GGGCCTCACAAGACAAA | N | 0.045263056 | 0.002361119 |
| | ACAGGAGCCAGAAGTAA | | | |
| | GGACTGAAGGAGAAGG | | | |
| 360 | GGGCTGTGGTAGTGGGC | N | 0.040952505 | 0.00208698 |
| | ATAGGCAGCGAGATATCC | | | |
| | AGTGGTAACAGTTGT | | | |
| 361 | CTGACAGTGAAGTGGCT | N | 0.031812492 | 0.039377154 |
| | GGTTACATCCGGCAAGC | | | |
| | GGGTGACTTCCATCAG | | | |
| 362 | ATGGAACTCAGCGCCGA | P | 0.023486826 | 0.001596429 |
| | ATACCTCCGCGAGAAGCT | | | |
| | GCAGCGGGACCTGGA | | | |
| 363 | TGTGGCGTATGCTGCTAT | N | 0.019702877 | 0.006223919 |
| | GTGAAGCAGTAGCTGCT | | | |
| | GTCATGGCCAAGGGG | | | |
| 364 | CCTGTCCTGGATGCCTCT | P | 0.00965454 | 0.033166702 |
| | GAAGAGAGGGACAGACC | | | |
| | GTCAGAAACTGGAGA | | | |
| 365 | CTACGCAGGTACAGCCG | N | 0.036186736 | 0.037343509 |
| | CCGCTTCCAGACCATTGA | | | |
| | CATCGAGCCTGACAT | | | |
| 366 | GTGCCACCTCCTGTCTAC | N | 0.026135094 | 0.020992809 |
| | TCATTGTTGCATGAGCCC | | | |
| | TGTCTGCCAGCCCA | | | |
| 367 | TTTTGGAGGTCATGGCG | N | 0.016208816 | 0.004585939 |
| | GGAGGATCACCTGAGGC | | | |
| | CAGGAGTTTGAGGCCA | | | |
| 368 | GGGGGTCCGCTGCCCGA | N | 0.036287687 | 0.000285317 |
| | GAATGGGAATTCTCTTCA | | | |
| | CTAGAATATGGAGAC | | | |
| 369 | TGCTGTTATCCCTGCCTG | N | 0.015086928 | 0.011591695 |
| | GTCCTCACACTCACCCAA | | | |
| | CAATCCCAAGGCCC | | | |
| 370 | AAACTGAGCCATGCTACC | N | 0.024163767 | 0.013934955 |
| | AGCATCCCAGGGTCTCC | | | |
| | AGCCTACAGATGAGC | | | |
| 371 | GCCTCCTGATCCAGCCG | N | 0.032812021 | 0.023593741 |
| | GGGCCCAGATTCCACTG | | | |
| | AGGTTAGAGTCCATTT | | | |
| 372 | GGCCAGTGACAGAGTTTA | N | 0.034574547 | 0.036925907 |
| | CCCTTGCCTCCTTTCTTG | | | |
| | GTCTGCCAGCTTTG | | | |
| 373 | GTGATCAGCAGGGAGTTT | N | 0.037307746 | 0.009998526 |
| | ATTTGAGGACATCAGTCA | | | |
| | CCTTTGGGGTTGCC | | | |
| 374 | GTCTCTATCTTCATGAGT | P | 0.01420409 | 0.004620793 |
| | GTGACTTGAGGTGTTGG | | | |
| | GATGGGTGAGGGAGC | | | |
| 375 | CTAGGTCCAGGGAGAAA | N | 0.035533969 | 0.00147624 |
| | AGGCAGTGGTTGGGGTT | | | |
| | ACTGGAAATTTTGCTC | | | |
| 376 | TCCCTGCTCTGGGAGCAT | N | 0.0054211 | 0.002647433 |
| | TGCTAGCCTTCTACCCCA | | | |
| | TCCCTGGATCCACA | | | |
| 377 | GGGGATATCTGCTCAGC | P | 0.006663772 | 0.004198389 |
| | CAATGGAAAATCTGGGTT | | | |
| | CAACCAGCCCCTGCC | | | |
| 378 | GGAAAGGCTGGAAGCTG | N | 0.047811244 | 0.040345325 |
| | CAGACAGGATCCCTAGCT | | | |
| | TGTTTTCTGTCAGTC | | | |
| 379 | CCCTCACCTACATTCCAT | N | 0.008188221 | 0.001371251 |
| | AGTGGGCCCGTGGGGCT | | | |
| | CCTGGTGCATCTTAA | | | |
| 380 | CTGTCATCATCTCCACAG | N | 0.027607379 | 0.033162134 |
| | CCCACCCATCCCCTGAG | | | |
| | CACACTAACCACCTC | | | |
| 381 | CCCGGGAGAAAAGACGG | N | 0.027178118 | 0.01768929 |
| | ATGGCAGGATCCAAGGG | | | |
| | GCTAGCTGGATTTGTT | | | |
| 382 | CCTGACCAAGCACCAGA | P | 0.024353366 | 0.027667304 |
| | GGACACACACAGGCGAG | | | |
| | AAGCCGTACACCTGTC | | | |
| 383 | AAGCGGGGAGAGGGTAC | N | 0.040332442 | 3.69206E−05 |
| | ACAATGGGTATCTAATAA | | | |
| | ATACTTAAGAGGTGG | | | |
| 384 | ACAAAGGGGCATGGGCC | P | 0.027479936 | 0.025075796 |
| | TCCAGCCTTTGCCCACAA | | | |
| | GTGCCTCAGTGCCCA | | | |
| 385 | CCAGAATGGAAGGGGGT | N | 0.043822125 | 0.000197108 |
| | GGGGATTTTCTGTTCCTC | | | |
| | CCTGGAGTGGGTGAG | | | |
| 386 | CATGGCAGTCGCTTGGA | P | 0.03431699 | 0.002803689 |
| | ACCCACTCACACCAATCC | | | |
| | AGTGACCGTGTGTGG | | | |
| 387 | CAGGCATGGCTTTGTTTC | N | 0.047591699 | 0.017559411 |
| | TGGTTTCAATCTGTTCTC | | | |
| | GTTCCTTGTACCGG | | | |
| 388 | TCTGGACTTGAACTCTGG | N | 0.024578957 | 0.003836375 |
| | CAAGAGATGCCAAAAGG | | | |
| | CATTGGTACCGTGTT | | | |
| 389 | GCCAGATATGCCTGTTTC | N | 0.02311552 | 0.010177508 |
| | CTTTTCCCAGCACCATGC | | | |
| | CTGTGGAGGGGACA | | | |
| 390 | AGAAAGGACCAGTGCCG | P | 0.027182494 | 0.030293534 |
| | TCACATCGCTGTCTCTGA | | | |
| | TTGTCCCCGGCACCA | | | |
| 391 | GGTCCCCATGTGCCTGTT | N | 0.011044731 | 0.006347093 |
| | GTTCAGCCCTCTCTCTTG | | | |
| | TTCCCTTTCTGAGC | | | |
| 392 | TTAATTGAGAGGGGCAG | N | 0.007626962 | 7.03433E−05 |
| | GGCTGGAGAAGGAGCAA | | | |
| | GTTGTGGGGAGCCAGG | | | |
| 393 | CCATGTGTCCCATCTCAA | N | 0.048432163 | 0.045676091 |
| | GCCACAGAGCAACTCAC | | | |
| | AGGGTACTTCACACC | | | |
| 394 | TTGCCTCCCCCAGCCCC | N | 0.004356057 | 0.006527945 |
| | CTCCCCATCAATAAAACT | | | |
| | CTGTTTACAACCACC | | | |
| 395 | GAGTTACAAGCACCAGG | N | 0.004416027 | 0.001451564 |
| | GGATGCTCTACATCAAGG | | | |
| | GATGCACCTTCAGTC | | | |
| 396 | GTGACTGCTGGCTCTGTC | N | 0.042905866 | 0.011264289 |
| | ACCTCATCAAACTGGATG | | | |
| | TGACCCATGCCGCC | | | |
| 397 | CCCTGCTTCCCGACACCA | P | 0.023332576 | 0.008044545 |
| | GCCTCATGGAATATGCAA | | | |
| | CAACTCCTGTACCC | | | |
| 398 | TGAGGTCCTGGAGTGCG | P | 0.02228292 | 0.002856177 |
| | TGAGCCTGGTGGAGCTG | | | |
| | ACCTCGCTTAAGGGCA | | | |
| 399 | GACAAGTCCCAAGATGC | N | 0.007992622 | 0.006093324 |
| | CAGAAAGGCAGTCTCCC | | | |
| | AAGGACCCACCATGCA | | | |
| 400 | GTTGCAAAGGTGGAGGG | N | 0.037410884 | 0.000373474 |
| | TTTTAGACTCTCATGCTT | | | |
| | CAGGTGCTGTCGGGG | | | |
| 401 | GTGCAGTGCTCTGAGGG | P | 0.046597272 | 0.017788559 |
| | GACAGACAAGGCTTGGG | | | |
| | TGTATATGCCAACCAG | | | |
| 402 | CGGAGAGTCACCCAACT | P | 0.043575131 | 0.000793079 |
| | GTGTGGAAGACAAGATG | | | |
| | CTCTCGACAGTTGCAG | | | |
| 403 | GGTGGACGACTGTGTTA | P | 0.039942233 | 0.023844026 |
| | CAGCCTTGGCTGCGCTA | | | |
| | GTAGCTGGCTTTCATG | | | |
| 404 | AAGGACCAGTGTCTCCCA | P | 0.033280802 | 0.000151296 |
| | GGAATCTGCAGGAGTCT | | | |
| | GAAGAGGAGGAAGTC | | | |
| 405 | AGGAAAAGTCTTGGCTG | N | 0.008672586 | 0.030940433 |
| | GACCCCTTTCCTGCTGG | | | |
| | GTGGATGCAGTGGTCC | | | |
| 406 | CTAGTGCTACAGCAACTG | N | 0.018812458 | 0.008197009 |
| | AGACAGCAACCAAGAGG | | | |
| | CAAGAAACCTGGGAT | | | |
| 407 | GGCACCCAAGGTTTCTGA | P | 0.019843061 | 0.004008385 |
| | TTCTGACCCAGCAGTGGT | | | |
| | CCTGAAGAGAGCTG | | | |
| 408 | CCCAGGGAGTGCTCGAG | N | 0.048620486 | 0.015664898 |
| | GCGCATCAGGCCCGTTTT | | | |
| | TTACCAGTTTATATC | | | |
| 409 | GGCCCTGAGGCTGTACC | P | 0.046580984 | 0.02100308 |
| | ATATCACTGACCAGGTCC | | | |
| | ATCTCTACCCCATTG | | | |
| 410 | TCATGCTGAACAGAAGG | N | 0.01430334 | 0.006971567 |
| | GCAAGAGAGGTGGATCT | | | |
| | GTGAGGGAAAAGACCC | | | |
| 411 | GCTACGTGTCCCTGGCAT | P | 0.012942887 | 0.01317341 |
| | TTTAGGTGTCGGTTGGGC | | | |
| | AGTCATGGATCAGG | | | |
| 412 | CACAGTAACTCCTGCCTG | P | 0.049853186 | 0.001586586 |
| | CAATCCCAGTACTTTGGG | | | |
| | AGGCTCGCTTGAGC | | | |
| 413 | ACAAGGTGCTAAAACAGG | P | 0.035761898 | 0.020667243 |
| | TTCACCCCGATACTGGCA | | | |
| | TCTCATCCAAGGCC | | | |
| 414 | TGCTTCCTCAGGGCCCA | P | 0.009459734 | 0.007734844 |
| | CCATTGAAGAGGTTGATT | | | |
| | AAGCCAACCAAGTGT | | | |
| 415 | GATCTTCAAAGCTGCTGC | N | 0.049569073 | 0.041894138 |
| | GGGCATTCTATGTCCCCT | | | |
| | TCCTGTCAGATCAG | | | |
| 416 | TGGGAGCGCCCGAATCT | N | 0.023760699 | 0.006556657 |
| | GGCTCGGCAGAATATCTC | | | |
| | TTCGACAAGCACACC | | | |
| 417 | CCTGCCCCTATCACTAGT | P | 0.029678174 | 0.04997287 |
| | GCATGCTGTGGCCAGAC | | | |
| | AGATGACACCTTTTG | | | |
| 418 | CAGTTCGGTTTTGGACTC | N | 0.045719947 | 0.002522644 |
| | TGAGTCAAAGGATTTTCC | | | |
| | TTTAAATGCTTGTC | | | |
| 419 | CCCCCACCCCCGAAAAT | N | 0.038002274 | 0.004756997 |
| | GTTCAATAATGTCCCATG | | | |
| | TAAAACCTGCTACAA | | | |
| 420 | CGGGACCTCAAGGTGAT | N | 0.012599874 | 0.005831922 |
| | GACAGATGTAGCCGGCA | | | |
| | ACCCTGAAGAGGAGCG | | | |
| 421 | AGCCCTTGGGCTCCCTTC | P | 0.036916791 | 0.013191742 |
| | TCTTTGATAGCAGTTATA | | | |
| | ATGCCCTTGTTCCC | | | |
| 422 | GGTTCAGAGAGGGGAAG | N | 0.032783796 | 0.000240423 |
| | TGATTGGCCTAAAGTCAG | | | |
| | GAACTAGGCAAGTGG | | | |
| 423 | TTGAGCCACGCATAGTGT | P | 0.018823519 | 0.00503824 |
| | CACGCACCTGTGATCCCA | | | |
| | GCTACTTAGGAGGT | | | |
| 424 | GCTCCAGGCCTAGGTGC | P | 0.049934442 | 0.009379965 |
| | CCAGGCTATGATGAGTCT | | | |
| | GCTTTTGAAGGAGGT | | | |
| 425 | CGCAATGAGTAGGGCTG | P | 0.022075229 | 0.036937961 |
| | GGTCTCGGCCATGGAAA | | | |
| | GCATACCTCAGTGCTC | | | |
| 426 | TCACGAACAATAGCTTGC | N | 0.008476161 | 0.005954972 |
| | GCTCTACTCTGTAGTTAT | | | |
| | GTGGATTGCCGAGC | | | |
| 427 | CAGGGGAATTCAACGAC | P | 0.038408863 | 0.021614244 |
| | CTGAGAAAGGTCACCAA | | | |
| | GGAGCAGTGGGACACC | | | |
| 428 | CCAGCAGAATGGGAATG | N | 0.047649343 | 0.000550068 |
| | GGGGAAACACAGCAGTT | | | |
| | CTTGGGTAAAAGTCCC | | | |
| 429 | GGAGGGCTGGAATCTGT | N | 0.003636844 | 0.010730638 |
| | CTTCCCTGACTCGGCTCC | | | |
| | TCAGGTCTTTAGCCT | | | |
| 430 | TGGATCAACAACTGCTAC | N | 0.043800643 | 0.042616071 |
| | TCTCGGGAAGACTCCTCT | | | |
| | ACTCACAGCTGAAG | | | |
| 431 | CTCTCTGATGCTGATTTG | N | 0.021003288 | 0.033603647 |
| | CACTCTGCTGGAATTCTG | | | |
| | CCTAGCTGTGCTCA | | | |
| 432 | ACAAATCAGCCTGGTCAC | P | 0.033117538 | 0.022037076 |
| | CAGCTTTTCGGAACAGCA | | | |
| | GAGACACAGAGGGC | | | |
| 433 | TGTGCCAGGGCCAAGGG | N | 0.042007277 | 0.001312877 |
| | GACACAGAAGATGGCAG | | | |
| | GATGACACTAATGGGG | | | |
| 434 | GCACACGTGCAGCCTATA | N | 0.033166623 | 0.017705348 |
| | TGGGAAAACCTTCCCTCT | | | |
| | GTACCTGCCTCAAA | | | |
| 435 | CATGACTCTGAACCGTAA | N | 0.036836062 | 6.98281E−05 |
| | CTGCATCATGAACTGGTG | | | |
| | ACGGGGCCCTGGGC | | | |
| 436 | TTCATCACCGAGGGCCT | P | 0.029047586 | 0.043721123 |
| | GGAGGTGCACTTCGTGG | | | |
| | AACACTACCGGGAGAT | | | |
| 437 | ATAGGTAACCAAGGAGCA | N | 0.046635012 | 0.00957964 |
| | GTGAACAGCAGCTACTAC | | | |
| | AGTAGTGGAAGCCG | | | |
| 438 | GGCGCGCGGCCAGGAG | P | 0.021029272 | 0.029965971 |
| | CACGCATGGTATTGACTT | | | |
| | AAAAGGTTCATTTTGT | | | |
| 439 | CTTTAGGGGTAGACTTAT | N | 0.023506379 | 0.000611306 |
| | ACCTTAAGTGAAGGAGTG | | | |
| | GGGGAGGAAGAAGC | | | |
| 440 | GGCTGCAAAGGGAATAG | P | 0.003613279 | 0.003245303 |
| | GCTGATGGAGCTGTTCTG | | | |
| | TATGGTACTGTGTGG | | | |
| 441 | GTGGGAGCCGTACTCTTT | P | 0.034190976 | 0.013199811 |
| | GCCCTTCTGCTGATGTCT | | | |
| | ATCTCATGTCTGTG | | | |
| 442 | GGCCGCGGGAGCCGCA | N | 0.046275311 | 0.002221781 |
| | CGCGGCGATATGGAAGA | | | |
| | GGAGGGCAAGAAGGGCA | | | |
| 443 | TCCATAAAGCTACCAGCT | N | 0.044788767 | 0.000618749 |
| | CCTCGCGAAGTGAACCT | | | |
| | GAAGGGCGGAAAGGC | | | |
| 444 | CCTAGGACGCCAAGGGG | N | 0.023231498 | 0.000667463 |
| | GAAAGGAGAGGGCGGAA | | | |
| | AAGGACCAGCGGGATC | | | |
| 445 | CAGCCCAGAGAGATTATA | N | 0.044773772 | 0.001207759 |
| | AGAACTGATGTGGCCAG | | | |
| | AGTGCCTACCCACTG | | | |
| 446 | TCATTGGAGGTTGGGAA | N | 0.036674829 | 0.006079723 |
| | GGAAGTGAGGAGAAAGT | | | |
| | GTTCTTGTTTAGTGTT | | | |
| 447 | TTCTCCCATTGACAGGAG | P | 0.018276619 | 0.001413291 |
| | CACTTGGCCCTGCCTTAC | | | |
| | CTGCCAAGCCCACT | | | |
| 448 | CCGCCACCACGCGCAGC | N | 0.009503129 | 0.005072368 |
| | CTATTCGCCGCACTAACT | | | |
| | GTGCCCGATGTGTGC | | | |
| 449 | ATGGCCAGGTGGGGCCC | N | 0.049828092 | 0.004474054 |
| | CTGGGGCAATGGCAGTG | | | |
| | GTAGAACGCTCAACTT | | | |
| 450 | CTGGAGAGCTGGACAGT | P | 0.037358989 | 0.01495552 |
| | GTTGGTTAGCTTCCTGCA | | | |
| | TTGATTGCCCCTGGG | | | |
| 451 | CCTAGACTTTGATTTCTC | P | 0.040909116 | 0.020653875 |
| | CGGCAGCCCAGATGTTC | | | |
| | AGTTCTCTTGGCCCC | | | |
| 452 | GCAAAATGACTGCAGCTC | N | 0.049921558 | 0.019239507 |
| | AGAAGGCTTTGGCTAAAG | | | |
| | TTGACAAGAGTGGA | | | |
| 453 | TTTTTGCAGGGTGCTGCC | N | 0.004556985 | 0.001659403 |
| | TATGGGCCCTCTGCTCCC | | | |
| | CAATGCCTTAGAGA | | | |
| 454 | GGTGGGTTTGCCTAGGG | N | 0.03482829 | 0.012773439 |
| | ACGTGTAACTACAGGCTT | | | |
| | TTACTAAGCCAAGGA | | | |
| 455 | CGTCAAGCATGCCTTCGA | P | 0.02353455 | 0.028088035 |
| | GATCATACACCTGCTCAC | | | |
| | AGGCGAGAACCCTC | | | |
| 456 | CCCTGCCCCCAAACTGG | P | 0.01003673 | 5.36219E−05 |
| | CTAAGACAGCTTTCAGTT | | | |
| | CCTGACTCCCCAACT | | | |
| 457 | AGGTCTCCTATGGGTGC | N | 0.023814896 | 0.005421136 |
| | CTGGGAAGTCCTTGAAAG | | | |
| | TGGACTGTTCTCAGG | | | |
| 458 | CCTTGAGGTAGAATGTGA | N | 0.009468898 | 0.006664989 |
| | GTCTCAGAAATGACTGCA | | | |
| | TTACCTGCcctttt | | | |
| 459 | GGCGGAATTGGGGGACT | N | 0.033767851 | 0.000318511 |
| | GTTTCCTGACATCCTGGA | | | |
| | CAAGGGAAGCCCACT | | | |
| 460 | AAGTCAGTGGTACACAGA | N | 0.021500535 | 0.003315181 |
| | CATTCTGTACATATCCTG | | | |
| | TGAAACGTGCTGTC | | | |
| 461 | CAAGACTGAATGGTGAG | N | 0.035483944 | 0.04277624 |
| | GCCAGGTACAGTGGCGC | | | |
| | ACACCTGTAATCCCAG | | | |
| 462 | GATGTGGGCCAAGTCCA | N | 0.029658841 | 0.021781126 |
| | CTGTCCTCCTTGGCGGC | | | |
| | AAAAGCCCATTGAAGA | | | |
| 463 | TTTATTCACGTGTTTGTTC | N | 0.038805705 | 0.011540941 |
| | CTGGTGGGCAAGATGCC | | | |
| | ATCTGAGGCTTCAG | | | |
| 464 | CCGTAGGGCATGTGGTT | N | 0.023419853 | 0.026276822 |
| | CAAAGAGAAGCAGGAGG | | | |
| | GCAAGGGAAAGTTACC | | | |
| 465 | GCTACGGCTCTGGACCC | N | 0.019002931 | 3.14784E−05 |
| | TGGAGTGGCTGCAGGCG | | | |
| | GCATGGGGCTGCAAGC | | | |
| 466 | GCTGTCCCTCCACTACAG | N | 0.044162526 | 0.005878851 |
| | AAACCTCACAGAACACAG | | | |
| | CAAAGGATAAGTGC | | | |
| 467 | GCAGCTCACTAGCCCAC | P | 0.013837108 | 0.002245382 |
| | CCCTCCTCTATTTTGGGT | | | |
| | AAGAGAATTTACTAC | | | |
| 468 | CCTGCCTGATGAGGGGA | N | 0.018119342 | 0.039635577 |
| | AACTACAGCACCTGGAAA | | | |
| | ATGAACTCACCCACG | | | |
| 469 | TGGATTTTGAAACTGTCT | P | 0.017451819 | 0.000494604 |
| | TGTACTGTCCTGGCAATG | | | |
| | GGACTGATGGTGCC | | | |
| 470 | GCCAGATGGTCATCATAG | P | 0.008787821 | 0.006574062 |
| | TCACGGTGGTGTCGGTG | | | |
| | TTGCTGTCCCTGTTC | | | |
| 471 | CAAACACCTTACAAAGTG | N | 0.031967844 | 0.000472304 |
| | CTGAGTAGGTAATAGTGA | | | |
| | CCCAACTTGTTTGC | | | |
| 472 | CTGGTGACCGCCACAGC | N | 0.024035432 | 0.017787617 |
| | CCCGCTTTGTAACCAGG | | | |
| | GAATACACAGTCATTT | | | |
| 473 | TCCTGCAAGGATATTGTG | N | 0.048483992 | 0.035045431 |
| | GCTGGAGACATGAGCAA | | | |
| | GAAAAGCCTCTGGGA | | | |
| 474 | GCATTTGCCAATTCAAGG | N | 0.032938336 | 0.008195673 |
| | TAAAACAGGGTCAGTGAC | | | |
| | ATCTGCAGTGTCCC | | | |
| 475 | CGCCTCAACTGCTGCCC | N | 0.005155787 | 0.007788042 |
| | CTGGTTGAATGTTCTCTT | | | |
| | GATAGTGCTGGACCC | | | |
| 476 | CCAATGCCAACCTCATGC | P | 0.016672504 | 0.001493406 |
| | GGAACGGGGCCGACTAC | | | |
| | GCTGTTTACATCAAC | | | |
| 477 | TTCCAAAGCAGTTAACCC | N | 0.049459764 | 0.007602317 |
| | AACTCCTAACAACATTTT | | | |
| | CGGGGGATCTGACC | | | |
| 478 | TGGGTTCCTGTTGCCCTG | P | 0.007419042 | 0.006278724 |
| | TAATTAAACTGCTGCCCG | | | |
| | TAGAGGCCTTTCAG | | | |
| 479 | CATCCAGGAACTGAGGC | N | 0.01324661 | 0.013272519 |
| | CTGAACCATTTTGCATTT | | | |
| | CCCCCTCCTCCAGCC | | | |
| 480 | TCAGGTGCCCTTATGAAA | P | 0.029857838 | 0.010754685 |
| | AGGCTTGATAGAGGGAG | | | |
| | TTTGTCCTGTGGCCC | | | |
| 481 | CTGCCATAGTTACCTGGA | P | 0.036961837 | 0.011147436 |
| | TTGTCAGCCTTGGTAGCC | | | |
| | TTTGTCTAAAGTCC | | | |
| 482 | GTAAGGGTGGCAAGGTC | P | 0.031164038 | 0.008886845 |
| | TTCACTCTGTGGTCATTC | | | |
| | CATGCAGAACATCAG | | | |
| 483 | AACACTGTGAAAGTTACT | N | 0.028568309 | 0.000463693 |
| | TGGGGAGGGTGGGCCG | | | |
| | GTGGGGCCGTAGCTCT | | | |
| 484 | TTCCTGGCCTCCCCTGAG | N | 0.01654291 | 0.027941991 |
| | TACGTGAACCTCCCCATC | | | |
| | AATGGCAACGGGAA | | | |
| 485 | TGATTTTGGGGTAGCAAT | N | 0.03525057 | 0.011430599 |
| | CCAGGAGAAGGTGCTGG | | | |
| | AGAGGGTGAATGCCG | | | |
| 486 | TCAGAGCAGTGGCTGGG | N | 0.041686823 | 4.36744E−05 |
| | GCACTGGAAGTATGTCCT | | | |
| | GGGGAGCAATGCATG | | | |
| 487 | CGCGGGATCCTTGTGCA | P | 0.032815159 | 0.004185415 |
| | GGGAAGAGCTGCCCTGG | | | |
| | GCACCTGGCACCACAA | | | |
| 488 | AGTGAGCCAAGCAGAAG | N | 0.040838073 | 0.030352253 |
| | GAGGTGGAAAACGGACC | | | |
| | CAAACCCCAGTGTGCC | | | |
| 489 | AGGCATCCGCCGCCCCG | N | 0.018038639 | 0.012964413 |
| | TGTCGCATCCTGGAATAA | | | |
| | AATGTGGCTCTGGCA | | | |
| 490 | GTTTCCTTTTTATCTCTCA | N | 0.029546481 | 0.040012925 |
| | AGCCACCAGCTGCCAGC | | | |
| | CACCACGAGCCAGC | | | |
| 491 | GCAAGCTTCCTCCCTCAG | N | 0.004607924 | 0.011592561 |
| | CCATTGATGGAAAGTTCA | | | |
| | GCAAGATCAGCAAC | | | |
| 492 | CAGTTTGATCCAAATAAA | N | 0.011465896 | 0.001926674 |
| | CAGACCCCGTCTGGCAA | | | |
| | GAAATGCATTGCAGC | | | |
| 493 | TGCCCACTCATTTGTATA | N | 0.042842123 | 0.011071869 |
| | AGTGCGCTTCGGTACAG | | | |
| | CACGGGTCCTGCTCC | | | |
| 494 | CCGGCTTCTGGGTCTTTG | P | 0.014758016 | 0.006353836 |
| | AACAGCCGCGATGTCGA | | | |
| | TCTTCACCCCCACCA | | | |
| 495 | TGTGGGCAACATCAGTCC | P | 0.047109149 | 0.023303175 |
| | CACCTGCACCAATAAGGA | | | |
| | GCTTCGAGCCAAGT | | | |
| 496 | TTCTCAATCCCCTGCTGT | P | 0.023796576 | 0.031236042 |
| | GGTAGGAACTCCAGTGG | | | |
| | TGAACGGCTTGCGCG | | | |
| 497 | CCACAAAGTGACCAAGAA | P | 0.024982993 | 0.001768943 |
| | CGTGAGCAAGCCCAGGC | | | |
| | ACAGCCGACGCCGCG | | | |
| 498 | CATGAAGCAGCTGGAGG | P | 0.006534179 | 0.000916375 |
| | AGTTGGAGGAGGAATTTT | | | |
| | GCCGCCTGAGACCCC | | | |
| 499 | CGTCTTGGTGCCTTTTGT | N | 0.022931693 | 0.04250559 |
| | GTGATGCGCCTTGCTGAT | | | |
| | GGCTTGACATGTGC | | | |
| 500 | TTGGTGGGGCTGGGTAC | N | 0.020120849 | 0.035717305 |
| | CAATGCTGCAGGTCAACA | | | |
| | GCTATGCTGGTAGGC | | | |
| 501 | GCCTTGTTGCCTCTGCCC | N | 0.046837754 | 0.012937732 |
| | TTTGAAGTCGGAACAATT | | | |
| | CCTAGCACCTGTCG | | | |
| 502 | ACCTCTTTTAACAGGAGC | N | 0.034667737 | 0.00461432 |
| | CTGAGCACAAGGTTTAAT | | | |
| | GAGGAAGCTGGGGC | | | |
| 503 | GTCCTGGGAAAGGAGTA | P | 0.026365815 | 0.012276163 |
| | CACCGAGAAGACCCCCA | | | |
| | TTTCTGAGCATGCTGT | | | |
| 504 | CAGCCAAGGGGAAAACA | N | 0.012289952 | 0.003564366 |
| | TGGCTCTTCTGCTCCAAA | | | |
| | AAACTGAGGGGGTCC | | | |
| 505 | ATGTTGGCGGTCCCTGT | P | 0.024624458 | 0.001969257 |
| | GACCTGTGGAGACACGG | | | |
| | CCAGATCTGCCCTCCA | | | |
| 506 | CCAAACACTCTCCCTACC | P | 0.042488377 | 0.0299422 |
| | CATTCCTGCCAGCTCTGC | | | |
| | CTCCTTTTCAACTC | | | |
| 507 | CACCATCATCCCAGTAGC | N | 0.010152256 | 0.019762924 |
| | TGCCCTATTCAACTGCAA | | | |
| | CAGTCTCCAGGACC | | | |
| 508 | CTGAGGAGCGAATCCGC | P | 0.046216056 | 0.007150319 |
| | AGTGGGGTCAAGAGGCT | | | |
| | GAGTAAGAGCCGCCAA | | | |
| 509 | TGTCCGCCATGGTCAGAA | P | 0.025520917 | 0.024886868 |
| | CACCTACCTCCCCTGGTT | | | |
| | ATTGTGAGGCTGGC | | | |
| 510 | GGTGGACGACTGTGTTA | P | 0.016042966 | 0.012228281 |
| | CAGCCTTGGCTGCGCTA | | | |
| | GTAGCTGCCTTTCATG | | | |
| 511 | GCACAGTCCCAGGTCCC | N | 0.006042082 | 0.001201442 |
| | AGCTCCCCTCTTATGGTT | | | |
| | TCTGTCATAATGTGC | | | |
| 512 | AACAGGGGAACTTTCCGT | P | 0.035708812 | 0.004734847 |
| | ACCTGCAGTGGGTGCGC | | | |
| | CAGGTCACAGAGTCA | | | |
| 513 | GCTGACCCCAGCTTCCA | P | 0.047054415 | 0.007023301 |
| | GGGGACTGTCACTGTGG | | | |
| | ACGCCAAAATGGCATA | | | |
| 514 | AGAGCCCCACACGGAGG | P | 0.008921035 | 0.019620598 |
| | CATCTGCACCCTCGATGA | | | |
| | AGCCCAATAAACCTC | | | |
| 515 | CATGGGCTGGGTTTTGTG | N | 0.00350231 | 0.002287415 |
| | CTTACTGTATGTTGGCGA | | | |
| | CTTGGTAGGGCAGG | | | |
| 516 | CAGTGAGCTGCCCCCAA | N | 0.037639269 | 0.04098076 |
| | ATCAAGTTTAGTGCCCTC | | | |
| | ATCCATTTATGTCTC | | | |
| 517 | ATCCCAGCAATTTGGAGG | N | 0.043034092 | 0.016140836 |
| | CTGAGGTGGGAGGATCA | | | |
| | TTTGAGCCCAGGAGT | | | |
| 518 | TGCTGGGTTCAAAGTCCT | N | 0.049871254 | 0.001679007 |
| | TAGAATTCCCTTCCTCCC | | | |
| | TCAACAAGCTGCTG | | | |
| 519 | GGGTGAGCTGCACCTGA | N | 0.013240539 | 0.018243242 |
| | TTAGTTGAAAGGCCTCAA | | | |
| | GAACAAACACTGCAG | | | |
| 520 | TTGGGGCCAAACACAGA | P | 0.018213436 | 0.001272422 |
| | ATCAGCAAAGAGGAGGC | | | |
| | CATGCGCTGGTTCCAG | | | |
| 521 | CTGTGTCTGGGTACCCTC | P | 0.007729048 | 0.003069837 |
| | TGAGTAGGCCTATAATTC | | | |
| | CTACCTTGACTGTG | | | |
| 522 | CCCTGCCCTCCACAGAAT | N | 0.023934013 | 0.031236757 |
| | TGGGTTCCAAGGGCTGTT | | | |
| | CCAGACAACTGCCA | | | |
| 523 | GTACAATGTTATCTCTGT | P | 0.02467355 | 0.022540344 |
| | GGGAGGAAGGAGGCAG | | | |
| | GCTGTGGTGGGACTGG | | | |
| 524 | GGACCGTGGAAGAACAG | N | 0.021551082 | 0.004250885 |
| | CACTTTCAAACCCACAAC | | | |
| | TGAGAATGATGACAC | | | |
| 525 | GAGACTGGGGTGCATCT | N | 0.021805161 | 0.002759933 |
| | CCAGAGCCACTCACACC | | | |
| | CTCAACCTCGTTTCCT | | | |
| 526 | GCAGAGCTGTGCTTCCT | P | 0.039666552 | 0.003300055 |
| | GGACGTGATTCCCTTTTG | | | |
| | GAAGCTGGACCTGGA | | | |
| 527 | ACTTAACCTGCTCAGGCG | N | 0.032952204 | 0.029580446 |
| | GGCCTTCGCCCAGCTGC | | | |
| | AAATAGGGATGCGTT | | | |
| 528 | TATAAGTGCCATTGTTGT | N | 0.009264582 | 0.000984511 |
| | AAGGTGGTGTTTCCTAGA | | | |
| | CCTTCCCTGATGCG | | | |
| 529 | GGGGAGCTTCCAATTAG | N | 0.021105438 | 1.79908E−05 |
| | CATACATAGACACATGTG | | | |
| | TCAGTGGCCAAGACC | | | |
| 530 | GAGGACGTCCCGGCTGG | N | 0.047966416 | 0.042357005 |
| | GATGAAGTCTGGTGGTG | | | |
| | GGTCGTAAGTTTAGGA | | | |
| 531 | GTGAGATTCGTGTTACTT | P | 0.02147264 | 0.009805861 |
| | TGGCTTTTCTGTCTCTGT | | | |
| | TGACACGGTTGCAC | | | |
| 532 | ATGTGTCACCTGGAGCTG | N | 0.015586966 | 0.017261609 |
| | GGCACTAACCATTCCAAG | | | |
| | CCGCCGCATCGCTT | | | |
| 533 | GATGTGGCGATCAGGGG | P | 0.04109581 | 0.005430416 |
| | ATAGTGAAGTTTACCAAC | | | |
| | TGGGTGATGTCAGTC | | | |
| 534 | TGAAGAGAGGAGGGGGA | N | 0.048100684 | 0.001272735 |
| | CTTTAGAGAGGGATGAAA | | | |
| | ATGAGCCCTGGGAGG | | | |
| 535 | GCAGGTCTTGTTAAAGCT | P | 0.033386333 | 0.006947321 |
| | TGCAGGCAGTGACACAC | | | |
| | AGAAGACGGCCGTGC | | | |
| 536 | CCTGGACCTTTGATGGAA | N | 0.020092936 | 6.366E−05 |
| | CAGATGGGAGGAAGAAG | | | |
| | AGGAGGACGTGGAGG | | | |
| 537 | GCAATCAGCATTCCTCCC | P | 0.000440445 | 2.08857E−05 |
| | CTGTGGATGACATAGAGA | | | |
| | GTCATGCCCAAACA | | | |
| 538 | TGGACCTGGGTCATAGG | P | 0.011505379 | 0.00307559 |
| | CTGAACCTGTTATGGACC | | | |
| | CCCAAATTCTGAGAG | | | |
| 539 | TGAGGACCTGTTCCCCG | P | 0.047900953 | 0.011956872 |
| | AGGCCAAGCAGAAACGG | | | |
| | CTTGCCAAGTCTCCAG | | | |
| 540 | TATCTTCCATTCCTCGCC | N | 0.01524656 | 0.028545498 |
| | CTGCCCCCAGAGGCCAG | | | |
| | GAGCTCTGCCCTTGA | | | |
| 541 | CGCTTCACGGAAATGCG | P | 0.026303229 | 0.001368521 |
| | CGAGATGGACCTGCAGG | | | |
| | TGCAGAATGCAATGGA | | | |
| 542 | GCAACAAAGTCCCGAGC | N | 0.019283435 | 0.015707034 |
| | AATGAAGTCATCCACTCC | | | |
| | TGCATCTGGTTGGTC | | | |
| 543 | CAGCCATAGCACTTTGTT | N | 0.003946328 | 0.000146371 |
| | CACTGTCCTGTGTCAGAG | | | |
| | CACTGAGCTCCACC | | | |
| 544 | CCAGGCTGTAAAAGCAAA | N | 0.041471932 | 0.001873204 |
| | ACCTCGTATCAGCTCTGG | | | |
| | AACAATACCTGCAG | | | |
| 545 | CCTGACACTGGAGGGCA | P | 0.04901318 | 0.02574041 |
| | GCTGTCTTGTGCATTACT | | | |
| | TGTGTTCCCAGCACC | | | |
| 546 | TTGGTGGTCCCGAGGGA | P | 0.033313644 | 0.022003999 |
| | GAACTGACCATGGACCT | | | |
| | GCACTCTTGTCCGAAG | | | |
| 547 | AGGTCTTGGGAGCTCTTG | N | 0.011121655 | 0.018991095 |
| | GAGGTGTCTGTATCAGTG | | | |
| | GATTTCCCATCCCC | | | |
| 548 | GGCTCTTATGGAGCCCT | P | 0.021014601 | 0.000290962 |
| | GGAGTTGTTGGGCAAGG | | | |
| | ATGCTGTCATTTTTTG | | | |
| 549 | TCTTGCCATGCAGCCCTT | P | 0.038103988 | 0.008797032 |
| | TTTATGGAGCAAGTCACA | | | |
| | ATACAGCAGCTTGC | | | |
| 550 | GCAGCAGCCAATTACAG | N | 0.046939746 | 0.032834718 |
| | CCCCTTTTGTAGCCGGG | | | |
| | CGTTCCTATGGTCAAA | | | |
| 551 | CTGTTGGCCTTTCTTAGC | N | 0.017576999 | 0.007364532 |
| | CTTTTTCTGTTTCCATTCA | | | |
| | GTGCTCCAAGCGC | | | |
| 552 | AGGGAAAGGGAAGTCGT | N | 0.042642576 | 0.003697907 |
| | GGAGAGGCAGGGAAAAT | | | |
| | GGTTAAGCAGCCCGGC | | | |
| 553 | ACTGGCGTTGCTGGGTG | N | 0.032734181 | 0.029268276 |
| | GCGATGCCCGTCCTCTG | | | |
| | GCTTGGGTTAATTCTT | | | |
| 554 | CCGGGGATTAACCAGCG | N | 0.046017403 | 0.03494442 |
| | TTATCAACCAGAAGCTAA | | | |
| | AGGATGATGAGGTTG | | | |
| 555 | ACCAAGGTGCAGAGATG | P | 0.003598978 | 0.013287415 |
| | GACAAGAGCAGCCAGGA | | | |
| | GACCCAGCGATCTGAG | | | |
| 556 | CTCCTTGATGGGCATGGA | N | 0.030375948 | 0.029680333 |
| | CTGGAAAGGATCCCAGG | | | |
| | AATACAAGAAGGCAG | | | |
| 557 | CTCAAGTCTGTATCTGCC | N | 0.040349518 | 0.015511437 |
| | TCCCCTGCCTTATTTCTT | | | |
| | ATGTTTTGCCACAG | | | |
| 558 | GCCAGGGCGGGGGCGG | N | 0.034459828 | 0.00226175 |
| | GGTGCATTTCCATCCTTG | | | |
| | TAAACCCTTCATAGTA | | | |
| 559 | AAAAAAGGATCAGCTGGC | N | 0.00507332 | 0.001092227 |
| | TGGGCAGGGTGGCTCAT | | | |
| | GTCTATAATCTCAGC | | | |
| 560 | CTGTGTTTAAGGCCGTGT | P | 0.028621069 | 0.00868039 |
| | CATTCAAGAGCCAGGTG | | | |
| | GTCGCGGGGACAAAC | | | |
| 561 | GCCCCAGCAGTGCCTTG | N | 0.002318217 | 0.011451138 |
| | TGCAATGTGAATGTGCGC | | | |
| | GTCTCTGCTAAACCA | | | |
| 562 | TGCCTTCCTTGACCATCT | P | 0.048912293 | 0.019934095 |
| | ACCTTGAACTAACCCACT | | | |
| | CCCAGCTCCCAGCC | | | |
| 563 | AGATACTCTTTGTGGGGA | N | 0.03332572 | 0.000651867 |
| | AGAGGGGCTGGGGCATG | | | |
| | GCAGGCTGGGTGACC | | | |
| 564 | CCACCCAAGGTATCAGC | P | 0.018610192 | 0.019657433 |
| | CGCAGTTGTGCCGCCAT | | | |
| | CATAGCCTACCTCATG | | | |
| 565 | GGAGGCCCTTCCCAAGG | N | 0.006884547 | 0.006288022 |
| | TGTGGTGACTGTGCCTTA | | | |
| | CTGTACATGCTCGGA | | | |
| 566 | GGTGGGGGCCGTTGCAG | N | 0.009732155 | 0.001598404 |
| | GTGTACTGGGCTGTCAG | | | |
| | GGATCTTTTCTATACC | | | |
| 567 | GCCTTTGGGTGGTGCAG | P | 0.022216512 | 0.003686331 |
| | TTCCCTCCGAAGACATGA | | | |
| | AATGACTCACACTGG | | | |
| 568 | AAATGGAGCCCTGAGGC | P | 0.011882824 | 0.001815474 |
| | ATCAGCTATTATACTTGG | | | |
| | GACTCTACCTCTCAC | | | |
| 569 | CCAGCTAAGCCCAGACC | N | 0.029137484 | 0.028129597 |
| | CCGTGGATTCTAGATAGA | | | |
| | TTTTAGAGGCAGCAG | | | |
| 570 | GTGATCATGCCACTGCAC | P | 0.013477333 | 0.000408422 |
| | TCCAGCCTGGGTGACAG | | | |
| | AGTGAGACCCTGTCT | | | |
| 571 | GGCAAAACACGCCCCCA | P | 0.014296774 | 0.006276019 |
| | TCTGCTGCCTTTTCTATT | | | |
| | GCCCTGCAACGTCCC | | | |
| 572 | TTATTTCTACTGTACATTG | N | 0.047780308 | 0.017617555 |
| | TTTTCTTTGTCCCAAGTT | | | |
| | GACCTAGGGTGAC | | | |
| 573 | CCTAGGGCCTTTTGTGGA | P | 0.035913481 | 0.017574484 |
| | TTGACAGTAGTCCCCTCC | | | |
| | GTAGGAGCTCACAG | | | |
| 574 | GAAGGGCATGGGGTGCA | P | 0.039267682 | 0.011593442 |
| | GCTAGAACAGTGTAGGTT | | | |
| | CCCTCAATAACCCCT | | | |
| 575 | TGGCCCGCACAACTTCC | P | 0.014196954 | 0.002359389 |
| | CCCTCGGGCTCCACTGC | | | |
| | CAGGTCAGATGCTGCT | | | |
| 576 | CTCTGAGCAGTCAGCGG | P | 0.012542104 | 0.005260003 |
| | CTCCAGTTTGGGCCCGAT | | | |
| | AAGGAAGTTCTCCGT | | | |
| 577 | AAGGAAGTGAACAGGCC | P | 0.042631636 | 0.021942582 |
| | TGACCCTGATGCCCAATA | | | |
| | ACGGGCAACCCTAGG | | | |
| 578 | GTCCACGTTTTCGGCAGA | N | 0.036290447 | 0.009871918 |
| | AGTAGTGAGTCAGTGTG | | | |
| | GAAGAGAGGTGAGGG | | | |
| 579 | GGTACCCGGCCAACATC | N | 0.040668273 | 0.008626021 |
| | AAGTGACTTTATAGCTGC | | | |
| | AAGAAATGTGGTATG | | | |
| 580 | AATGCCACTGACCCTTCT | P | 0.018093647 | 0.001122555 |
| | GCCCGCACGCTGACCCA | | | |
| | CTACAAGTCAGCTGT | | | |
| 581 | GACCCTAGCACTGGCTG | P | 0.04900488 | 0.002334043 |
| | TGACATGCTGCTTGGTGC | | | |
| | TGCCTCTGGTCCTGA | | | |
| 582 | GCTGGAGCCCGCACCCT | P | 0.031880914 | 0.00912872 |
| | AAGCATCCTGCTGCCTTC | | | |
| | CCACAACATTAAACT | | | |
| 583 | GGTGGTAACTCCCCAAC | N | 0.048370423 | 0.004896163 |
| | CTGACATTGGTACTGTGC | | | |
| | AATAAAGACACCCCC | | | |
| 584 | CCTCAGGATGGGGAGCT | N | 0.00703963 | 0.009747108 |
| | CACTCCGAGAACAGGAG | | | |
| | AAATCAACATTGCAGT | | | |
| 585 | CCCTCATGCTCCTTGTTT | N | 0.028886778 | 0.007616024 |
| | ATAGTCACAACCTCTCCT | | | |
| | CAGCCCTAACCCTT | | | |
| 586 | AACTGCTGGCCAACTCTT | P | 0.001857117 | 2.78336E−05 |
| | ACATCCCCAGCAAATCAT | | | |
| | CGGGCCATTGGATT | | | |
| 587 | CCCTCACAGCCCCCAAAA | P | 0.039339724 | 0.017153019 |
| | AATCTGCAACCCAGTTTA | | | |
| | CCGGTCGCTACCAT | | | |
| 588 | CCGGCCCAGCCAGGCCA | N | 0.023073918 | 0.001903273 |
| | TCTCACGTGTACATAATC | | | |
| | AGAGCCACAATAAAT | | | |
| 589 | GGCCACACGCAGAGGAG | P | 0.014794988 | 0.000543661 |
| | AAATGGTCTTATGGGTGG | | | |
| | TGAGCTGAGTACTGA | | | |
| 590 | GCGGTAACCAGCGTGAG | P | 0.036364137 | 0.017309332 |
| | CTCGCCCGCCAGAAGAA | | | |
| | TATGAAAAAGCAGAGC | | | |
| 591 | CCCAGAACACACATCTGA | N | 0.031578554 | 0.012764959 |
| | GACAGGACCATAATTATT | | | |
| | GTAGCCCCACCAAG | | | |
| 592 | GTCAGAGCAGTGCCACC | P | 0.043475944 | 0.007226452 |
| | TCCTGTCTCAGTTCTTCA | | | |
| | GAGAGCTTCCACCAG | | | |
| 593 | AGTGCCACATTTGGCAGT | N | 0.034104006 | 0.015924288 |
| | ACAAATGAGTCTGAGTGT | | | |
| | AATAGCCCAGAGAT | | | |
| 594 | GCCCTGGTGTCCTGGGT | N | 0.039047457 | 0.009406113 |
| | TTTCGTGATGATCTTTGC | | | |
| | TCTGTTTCCAGTGGG | | | |
| 595 | TTGGGGAGAGAAGAAAG | N | 0.034275174 | 0.004299453 |
| | GGGGTTCAGAGGCCGGT | | | |
| | ACCTCCCCTACAGCAG | | | |
| 596 | CTGCCTGCTGCATCTTTT | P | 0.012433872 | 0.001085221 |
| | CTGTTGCCCCATCCACCG | | | |
| | CCAGCTTCCCTTCA | | | |
| 597 | GCATGGGTTTTTCTGCAG | N | 0.025540155 | 0.008487738 |
| | CTTATTGTGAATACCTTG | | | |
| | GTTCTGTTCAATAG | | | |
| 598 | GTCCACGCAAAGCTTGG | P | 0.0056957 | 0.000592579 |
| | GGATTGTATTTGGACCTA | | | |
| | CCCTTCTGCGAGCTG | | | |
| 599 | GGTCCTGTACGACATTTA | N | 0.015088545 | 0.002437683 |
| | CTGGTCTGAGGAGATGG | | | |
| | AGGACCGGCTGCAGG | | | |
| 600 | CAGAAAGCCTGCTGGGG | P | 0.015476971 | 0.000810364 |
| | ATGGTGCCAGCTGTGCC | | | |
| | TTGGCTGTTGTATTTG | | | |
| 601 | GGTTTCCTTCAGACAGAT | N | 0.002087964 | 0.002531764 |
| | TCCAGGCGATGTGCAAG | | | |
| | TGTATGCACGTGTGC | | | |
| 602 | GCGCTGGGCAGAAAGGC | N | 0.039780854 | 0.023882913 |
| | TACCATCACCGCACTGAG | | | |
| | ATCAACAAGAAGATT | | | |
| 603 | TGACTCAAGGGCTGTAGA | P | 0.026592536 | 0.015754478 |
| | TGTTCCCTTTCCACCCCC | | | |
| | CACACTTGGTGCGT | | | |
| 604 | CGACAGCAGGACATACAT | P | 0.040247578 | 0.049282971 |
| | GTTGGTGTGAAGACTGG | | | |
| | GACGACACTGGGTAG | | | |
| 605 | CACCCTGTTCTTCCCTGG | P | 0.018338871 | 0.021118585 |
| | TATGTTGGCAATTCCCTG | | | |
| | CGCATGCTAAGAAA | | | |
| 606 | GTTAGAGGCCAACAATTC | N | 0.026696397 | 0.002804317 |
| | TAGTATGGCTTGTTGGCA | | | |
| | AAGAGTGCTACACC | | | |
| 607 | GCGCCTCAACTGCTGCC | N | 0.011833184 | 0.006401904 |
| | CCTGGTTGAATGTTCTCT | | | |
| | TGATAGTGCTGGACC | | | |
| 608 | CCTTGATACCCACTTTTT | P | 0.023692962 | 0.016952204 |
| | GTGAATACCTACCCCCTT | | | |
| | CTGGCCACGGTGCC | | | |
| 609 | ACCAGGTCCAATGTGGG | N | 0.039784026 | 0.000485212 |
| | GAGAAATATGAAGGAGGT | | | |
| | AGCAGCCCTGGGTTC | | | |
| 610 | AATGTGTAGGAAAAGCCT | P | 0.047807766 | 0.000887484 |
| | TCTGGCCACCCAGAGAG | | | |
| | CCGGAGCCCTGCTTG | | | |
| 611 | GGGACTATCCAGATCTTG | N | 0.019925486 | 0.023122196 |
| | TGTGGCATGATGGTATTG | | | |
| | AGCTTGGGGATCAT | | | |
| 612 | CATGCACCTACACTCCAG | N | 0.019590875 | 0.014811948 |
| | CCTGGGTGACAGGGCCA | | | |
| | GACTCTGTGAAAAAA | | | |
| 613 | CGTAGACAGAGCTAAAAG | P | 0.016167098 | 0.005508736 |
| | GAGTGGTGTGGCGTACA | | | |
| | TTGCGGCTCCCTCCG | | | |
| 614 | GCAGCTCCCAAGGACAC | P | 0.045662136 | 0.003180791 |
| | AAGCTGTTGGGATGCTAC | | | |
| | TTCTCAGCTTCACGC | | | |
| 615 | GCCACAGTCTAATGTCAT | N | 0.033271306 | 0.034462768 |
| | TCACTTCATTTGATGGGG | | | |
| | TCACTTGTTAGCTG | | | |
| 616 | GACCTTGCTAAGGCTAAA | P | 0.047412948 | 0.024480004 |
| | GAATCCCTGAAACAGGC | | | |
| | GCGTCATTCTCTCTG | | | |
| 617 | CCAGTGCCTTGGAACCT | P | 0.033210931 | 0.013227704 |
| | GCTGCCTTGGGGACCCT | | | |
| | GGACGTGCCGACATAT | | | |
| 618 | ATCTGCTGCCTATTAGGT | P | 0.010454101 | 0.001023498 |
| | TCTTCTGTGACATGTGCC | | | |
| | TCCCAGCAGTGAAC | | | |
| 619 | GCTCTTGGCTCCCTTGAG | N | 0.017690891 | 0.014305978 |
| | GTTCTGCTAGTGGTGTTA | | | |
| | GGAGTGGTTACAAC | | | |
| 620 | TGGCCCACCAACCCGCC | N | 0.018951587 | 0.005594844 |
| | CACCTTGACTGCCCCCA | | | |
| | GAAGTGTGTCTTGAGA | | | |
| 621 | GGCTGCCCCTCTTGAAC | P | 0.024962034 | 0.007363553 |
| | CACCCACATGCTTAGCCC | | | |
| | CAGCTTTTTGGAAGA | | | |
| 622 | CGCCAGTCAGAACCAATC | N | 0.047887627 | 0.003069205 |
| | CGAAAAGAATGTGTGTTG | | | |
| | ACTCAGGTTTGGAG | | | |
| 623 | CCTGCCCCAAACCGATAA | N | 0.029184491 | 0.017082703 |
| | GTGCATGACGGAACAATA | | | |
| | GGACTCCCCAGGGC | | | |
| 624 | ATCTCTCTTTGTCTTGAG | N | 0.02072694 | 0.000130379 |
| | GGGGCTGTTGAGTGGTC | | | |
| | GCGGACCTGGGCTCT | | | |
| 625 | GTTGACCAGAATAGTTGG | N | 0.030104875 | 0.023521738 |
| | TAACTCTGCCAGAGCCTG | | | |
| | TACTTACCTGCCAA | | | |
| 626 | GTTCCTGTCTCTTGCCAA | P | 0.032096251 | 0.004901309 |
| | CAAGAGGTTACCAGTGAA | | | |
| | AAGAGCTGCTGTCC | | | |
| 627 | GGAGAGTGCAGAGAAGG | P | 0.011986695 | 0.003276807 |
| | AGATCGGCTTGTGGTTTC | | | |
| | ACCCTGAGGAACTGG | | | |
| 628 | GATGGGGTACTGCCGGT | P | 0.014637429 | 0.003246485 |
| | CTGTGTTGTCTTAGAAGT | | | |
| | AGATGTGTGATGGCC | | | |
| 629 | GCCGACTCCCAGCCCGG | N | 0.048334671 | 0.046887347 |
| | CCAAAAAGACAAAACACA | | | |
| | TAGACGCACACACTC | | | |
| 630 | CTCCTCTTCCTTTCTCCG | N | 0.018512639 | 0.003766475 |
| | CCATCGTGGTGTGTTCTT | | | |
| | GACTCCGCTGCTCG | | | |
| 631 | ACAGGAAGCAAACTAAGC | N | 0.017028378 | 0.010897747 |
| | CCCCGCTGTAATGAAACA | | | |
| | CCTTCTCTGGAGCC | | | |
| 632 | GGGAGTACAGTGCCATTA | P | 0.038865142 | 0.009213826 |
| | GGACCAGCAAGTGACAC | | | |
| | AGGAATTCTGAATCC | | | |
| 633 | AAGGCTCGATCCTGCGA | N | 0.011236173 | 0.024519108 |
| | GAAGGAAGTGGTCTCTG | | | |
| | CCCAGCCTGCCACCTT | | | |
| 634 | GAACCCCAGGAAAAGGA | N | 0.021001888 | 0.013420666 |
| | AGAGGTCGAACCAACCC | | | |
| | TGCGGAAGGAGCATGG | | | |
| 635 | TTTTTAATCTGGAGCGGG | N | 0.035319468 | 0.000116512 |
| | GTGGGGAGTGGGAACCG | | | |
| | GAGAGAAAGCAAAAT | | | |
| 636 | GCCGCCGGGCGTGGTTT | P | 0.036581556 | 0.004069903 |
| | GCTTCTCTCAGTCACTGC | | | |
| | TGGCGCTTTCTTAAT | | | |
| 637 | CACCTGTTATCACAGCAC | N | 0.024220194 | 0.003865777 |
| | TTTGGGAGGCCAAGGTG | | | |
| | GGAGGACTGCTTGAG | | | |
| 638 | GCGCCCCCTTCTTCCAGA | N | 0.026812018 | 0.023037715 |
| | TGTAATAGAAAGCTCTGC | | | |
| | CCTATCACAAGGGG | | | |
| 639 | CCATGCTTGGGAGAATG | N | 0.020797587 | 0.000260101 |
| | GGGCTGGGACATGCACT | | | |
| | GAGTGTTGCACTTTTA | | | |
| 640 | CCAAACATGAGGAGGAA | N | 0.042239012 | 0.008205958 |
| | GAATGGACTGATGACGA | | | |
| | CCTGGTAGAATCTCTC | | | |
| 641 | GGCTTCAAGAGGCGGGC | N | 0.049462336 | 0.010042642 |
| | GTGCCCTCTGGAGTCCC | | | |
| | CTACCATGACTGAAGG | | | |
| 642 | ACATCATCCCAGAGGTG | P | 0.031955969 | 0.029382214 |
| | GTGTTCAGTGTCAGACCT | | | |
| | CTTAATGGGCCAGTG | | | |
| 643 | GCTTTCAAGGAGAGGCA | N | 0.026271755 | 0.017401357 |
| | ACGAGAAAGTTGGCAGG | | | |
| | AAAGTGACGCCATCCC | | | |
| 644 | CTTACTCTACTGATTTCC | N | 0.025293598 | 0.028612508 |
| | CCCCAGACTGTGATTTTT | | | |
| | GAACTTCCTTGCCT | | | |
| 645 | GGTGGATAAGGTGACTC | N | 0.028555911 | 0.007898531 |
| | AAAGGAAAGACGACTCAA | | | |
| | CCTGGAACTCAGAGG | | | |
| 646 | ATTTCCACATTTGGGGGC | N | 0.038788638 | 0.000556117 |
| | TATTCTAAGGTGACTATG | | | |
| | CCAGCCAACCTGGG | | | |
| 647 | GCAGAAGTGGCTGCTAG | P | 0.019876771 | 0.023622651 |
| | ATCTCTCTCCTTTACCAC | | | |
| | CATGCAGGAACAGAC | | | |
| 648 | GAGGAAGCCAGGACACG | N | 0.043002325 | 0.002980164 |
| | GAAGTGAGAGACACTAAA | | | |
| | GAAGGTGTGGGCCAG | | | |
| 649 | TTCATCAGCCCGCCAAGA | P | 0.033189724 | 0.018430306 |
| | TGGCGATGCAAGCGGCC | | | |
| | AAGAGGGCGAACATT | | | |
| 650 | CATCGTCTATGCTTCCCT | N | 0.007159022 | 0.003174055 |
| | TGCCCTCTCCAGCTCCAC | | | |
| | CTCACCCAGAGCAC | | | |
| 651 | GTGAACACAGGCATGGC | P | 0.040192458 | 0.006633996 |
| | GGCAGAAGTGCCAAAAG | | | |
| | TGAGCCCTCTCCAGCA | | | |
| 652 | CCTCTTGTCAGAGATCCT | N | 0.007334167 | 0.005849462 |
| | CTACCACAGACATTAATA | | | |
| | GCTGAGCAGGAGCC | | | |
| 653 | GTCACCCTGTTGCCTATG | N | 0.018396204 | 0.028552569 |
| | GGAGGAGGTGGAAGGAT | | | |
| | TTGGCAAGCTCAACC | | | |
| 654 | CCTGAGACTGGCAAGGG | N | 0.049033322 | 0.000573786 |
| | AGGAGGCTGAGCAGAAG | | | |
| | GAGTCATCATGGAGGA | | | |
| 655 | TGATCAGCTCTGAGGTGC | N | 0.036101964 | 0.002977882 |
| | AACTTCTTCACATACTGT | | | |
| | ACATACCTGTGACC | | | |
| 656 | GTCCAAGCTGTGCCTCG | P | 0.013805766 | 0.039331078 |
| | ACACATCCTCATCCCCAG | | | |
| | CATGGGACACCTCAA | | | |
| 657 | CCACCATGACCCTCTTCC | N | 0.026316937 | 0.000370002 |
| | TCATGCTGAACTGCATTC | | | |
| | CTTCCCCAATCACC | | | |
| 658 | GCCCTCCTGCATTGCTGC | N | 0.031465018 | 0.035461566 |
| | TGCGTGGGTATTTGTCTC | | | |
| | CTTAGCCATCAGGT | | | |
| 659 | TCTCCGCTCTGACTTGTG | P | 0.035874589 | 0.007190978 |
| | GCTCAGGACTACTTTCTG | | | |
| | GGTCGTGCTCCTGC | | | |
| 660 | GCAGGGGCTTAAATTCAC | P | 0.014094762 | 0.000684551 |
| | TGGAATCCACCCTGACTT | | | |
| | TTCCTGCCAGTACT | | | |
| 661 | AAACAACCCTGAGCCCA | P | 0.026292222 | 0.005231064 |
| | GGGCCAACCCCTACGGA | | | |
| | ACACCACTACTTTACC | | | |
| 662 | GCTTTCATGTCCCAGAAC | N | 0.001915146 | 0.007299795 |
| | TTAGCCTTTACCTGTGAA | | | |
| | GTGTTACTACAGCC | | | |
| 663 | CCGTGAGCAAAGATACTT | N | 0.024730379 | 0.03395056 |
| | CTTGGAATGGCTGCAGT | | | |
| | GAGGCCGTGTCATTG | | | |
| 664 | ATATGTGGTCCTCGTTCA | N | 0.033162464 | 0.017629 |
| | TCGTGCCGCCTGTGGTG | | | |
| | ATGCGTGCAGTGACG | | | |
| 665 | CCCAGGGCAGGTTTGCT | P | 0.025468392 | 0.021295769 |
| | TGACCTCTGCCTCAGTTC | | | |
| | TCGACTCTAAAGGAC | | | |
| 666 | CAGAGTTGAAAGCCACA | N | 0.037458876 | 0.007652731 |
| | GAGTTGAAAGCCACAGA | | | |
| | GTTAAGTGACCTCAGG | | | |
| 667 | CAGTACACATCCTATGCC | N | 0.004984238 | 0.011955861 |
| | ATAAGTGGTCCCACAGTA | | | |
| | CAAGGTGGCCTCCC | | | |
| 668 | CCTCTGAGTCATTGTGAG | P | 0.02879657 | 0.016233118 |
| | CTGTGTGGTAGGTTGGA | | | |
| | CATTGGCATAGTTGG | | | |
| 669 | GCCATACCTGGTGTCCAA | N | 0.033848292 | 0.030034111 |
| | AGAACTCATGAGCCTTGT | | | |
| | GTCTGGGCTGCTGC | | | |
| 670 | AGTGCCATTCACCACTGC | P | 0.034730466 | 0.00680122 |
| | GTCCTGGGCTTTACGAGA | | | |
| | CCATGCAAGACGGG | | | |
| 671 | CTGCCAGGATGGACTCG | P | 0.048331808 | 0.04348284 |
| | CTGCTCATTGCAGGCCA | | | |
| | GATAAACACTTACTGC | | | |
| 672 | ATCACCCGGACACACATC | N | 0.024107496 | 0.023689683 |
| | ATGCTTAAGATTCAACTG | | | |
| | GGAGCATACCAGGG | | | |
| 673 | GAAGACAGAAGAGTTCTT | N | 0.007514321 | 0.000131333 |
| | CTCTGTGGTGACTACAGA | | | |
| | CTAGAGGAATGCTC | | | |
| 674 | GAGCCAGATCCGCAGTG | N | 0.046493762 | 0.018187287 |
| | GTGGAGAGTTCTAATGTT | | | |
| | GACTGTTTGCAGGCC | | | |
| 675 | AAAGTCACTGAGGAAGAA | N | 0.02027623 | 0.004621778 |
| | AATAAGGGTTCAGGTACA | | | |
| | GGTGCAGCCGGTGC | | | |
| 676 | GTCACTGTGCCACTTGGA | P | 0.023850554 | 0.000597694 |
| | CAGGGCGTTTTCTCTGAA | | | |
| | TTGAAGGGAAAGCC | | | |
| 677 | TGCCTTCAGCCATTGAGT | N | 0.00714724 | 0.012463499 |
| | GGAAGCTGCCCCAGGCC | | | |
| | CTTACCAGGTGCAGA | | | |
| 678 | ACTTCTACTTGCCAACAT | P | 0.039625143 | 0.01205671 |
| | CTGCCTTGCTGGACTTGT | | | |
| | ATGGGATTGTCTCC | | | |
| 679 | GCTATGAGTTTGTGCCTT | N | 0.010550497 | 0.025286942 |
| | TGCTGAGGACACTAGAAC | | | |
| | CTGGCTTGCCTCCC | | | |
| 680 | TGAAGAAGTGCATCAGGT | P | 0.04541531 | 0.00171692 |
| | GCCAGGTGGTCGTCAGC | | | |
| | AAGAAACTGCGCCCA | | | |
| 681 | TGGGGGAGGAGACCCTT | P | 0.041700662 | 0.006501658 |
| | GGAAAAGTCCTCTCTTCC | | | |
| | CAGCTCCTGATTCTG | | | |
| 682 | GAGCTTGCCAGTGGTGA | N | 0.017926633 | 0.003860015 |
| | CTGCCAGGAACGTCCTAT | | | |
| | GATCCACTTTGTTGG | | | |
| 683 | GTCTGCATTTTGGAGAGT | N | 0.038703549 | 0.021196778 |
| | CCACACCACGGACCAGG | | | |
| | TTTTCCCCCAAGGCT | | | |
| 684 | GTCACATTGTCAGAACAG | P | 0.017221878 | 0.021757129 |
| | GTGGCTGCTGTGTGGTG | | | |
| | CCATCGAGTCCCTGC | | | |
| 685 | GTCACCTCTGAGGTGCCT | P | 0.018234848 | 0.018727928 |
| | GCTGGCATCCTCTCCCC | | | |
| | CATGCTTACTAATAC | | | |
| 686 | TGCACTGACTGGGCCAC | N | 0.035001782 | 0.005222941 |
| | ATCTTGAAAACTCGTGGA | | | |
| | AAGGGAAAGGGGAGG | | | |
| 687 | TCGGGGTCAGTTAAGCCT | N | 0.049510203 | 0.011185124 |
| | CAGTATTCTTAGCTTTTGT | | | |
| | TGATTTTGGCACT | | | |
| 688 | CAGGTGGCTCAGTTCCT | N | 0.044438194 | 0.008047993 |
| | GAAGGCGGCTGAGGACT | | | |
| | ATGGGGTCATCAAGAC | | | |
| 689 | TTTCTATTGGTCCTGGGT | N | 0.026599568 | 0.000220576 |
| | ACCGAGCATGGGCGCTG | | | |
| | CGTCAGTGCGCGCTG | | | |
| 690 | GTCCTCTCCATCATCCTC | N | 0.001283646 | 0.003138194 |
| | AGTGTGAGTCCTCAGAG | | | |
| | CCTCCATCTGCCAAG | | | |
| 691 | GGGGAAGGATAGGGTGA | N | 0.00664446 | 0.00391409 |
| | TGTTCCGAAGGTGAGGA | | | |
| | GCTTGAAACCCGTGGC | | | |
| 692 | AACTCTACAGATGGCATA | N | 0.011045234 | 0.003419594 |
| | CCTGTGCCTGCTTCTGG | | | |
| | GGTTGGAAGTGTGAC | | | |
| 693 | GACGAGCCCAGCTGCTA | P | 0.022483531 | 0.005532782 |
| | GATATCAATGGGAAGCTG | | | |
| | CGGGAGATGCTGAAG | | | |
| 694 | CGTACACACATAAACACA | N | 0.01777347 | 0.022420049 |
| | CCCACCAGTGCAGCCTG | | | |
| | AAGTAACTCCCACAG | | | |
| 695 | GCCTTACTCTGGAAGCG | P | 0.035452489 | 0.001688661 |
| | GCGAGCCGAGGCAGCCC | | | |
| | TTGATGCCCTTGACAA | | | |
| 696 | TGGACCCTTCCTATTGGT | N | 0.034302376 | 0.000905278 |
| | CTGTCCTGGGCCAACTG | | | |
| | GTGGGTGATCTCTGC | | | |
| 697 | CTGAGCCTGGGTGCTCA | N | 0.004168755 | 0.006982257 |
| | CTGTGGCGGTCCCCGTC | | | |
| | CTGGCTATGAAACCTT | | | |
| 698 | GGCTGAGGTGTTTTGAG | N | 0.020654317 | 0.003198734 |
| | GTGCATCGAAGTGTTCCA | | | |
| | AGCTGTGACTTACCT | | | |
| 699 | GGATTCCTGAGTTACTGT | N | 0.02072767 | 0.004364026 |
| | TTTGTTCCTCCCCACTGC | | | |
| | TTCCCATTCCTGAG | | | |
| 700 | CGTATAAAACTAAGCTGT | N | 0.029692664 | 0.001704682 |
| | GCCCCAACCACGCTGAC | | | |
| | CATGTCATCAGGACC | | | |
| 701 | AGGGTCACCACTGCCAG | P | 0.014082881 | 0.004438034 |
| | CCTCAGGCAACATAGAGA | | | |
| | GCCTCCTGTTCTTTC | | | |
| 702 | GTTCAGATGACAGCGAC | N | 0.016728142 | 0.004693716 |
| | CGCCTTTTCATTCCCCCC | | | |
| | GCCACCTGTACTCAC | | | |
| 703 | GCATTTCAGATGCTGTTG | P | 0.009719871 | 0.00526514 |
| | GACTTCATGTCCCCAACC | | | |
| | TAGCTTGGTGAGGG | | | |
| 704 | TCAGGCTCATTTGTACTC | N | 0.034663126 | 0.025017401 |
| | TCTTCCCCTCTCATCGTC | | | |
| | ATGGTCAGGCTCTG | | | |
| 705 | TTCAGTACTGTATATTTCA | N | 0.015558832 | 0.007106174 |
| | CCCTGTGTAATGGGGCC | | | |
| | CCCTCTCCTTTCTC | | | |
| 706 | GGTGCAACTTTGAGTCCT | P | 0.042286127 | 0.043739041 |
| | TGGCTTGACTATACAGGC | | | |
| | CTTGAACTTCATGG | | | |
| 707 | TCTGAATTTCCACTGCTT | N | 0.025554074 | 0.007973937 |
| | TGGAGAGTCCCACCCAC | | | |
| | TAAGCACTGTGCATG | | | |
| 708 | GGCCACCATCACAATACT | P | 0.048200787 | 0.025584934 |
| | AAGGGGCTCAGATGTGT | | | |
| | CTTGTGCCCACCTCT | | | |
| 709 | GGAGAACGTGCCCTATTC | N | 0.005323906 | 0.00886515 |
| | ACACTCTGGGAAGACGC | | | |
| | TAATCTGTGACATCT | | | |
| 710 | GCACGTGACACAAGCTG | N | 0.037138581 | 0.011351082 |
| | ACCTCAAGAGCTCCACGT | | | |
| | TTTGGCTTCGAGCAA | | | |
| 711 | TCGACTTCTGCGATCCCC | N | 0.027230804 | 0.016978713 |
| | TGGAAAACCAACCACTCC | | | |
| | CCCTGGGACGGTAA | | | |
| 712 | GTGTGATCTGGTCACTAT | N | 0.032492029 | 0.021879388 |
| | GTGACTGCCTTTACGGTT | | | |
| | TCTCTCCATGTGCT | | | |
| 713 | ATGCGGGTAGTATTAGCC | N | 0.001306922 | 0.00026264 |
| | ACCCCCCTCCATCTGTTC | | | |
| | CCAGCACCGGAGAA | | | |
| 714 | GCCTGGGTTCAAATCTGC | P | 0.035505176 | 0.012950353 |
| | ATCTGCCATGCACTTGTT | | | |
| | TCTGACCTTAGGCC | | | |
| 715 | GACCAACAGCAGAGATAT | P | 0.04288474 | 0.035207827 |
| | GCCTTTCTCATTCTTGGC | | | |
| | TACAGCAGTGGGAC | | | |
| 716 | CTTCCCCCTCACCCCCCA | N | 0.034683728 | 0.013940908 |
| | CTCTTAGGTATGTATGAT | | | |
| | GCTAATCTTGTCCC | | | |
| 717 | TGGCTGCCCTGATTCCAA | N | 0.031621213 | 0.01701817 |
| | GTGCTCTTATCGCCTCTG | | | |
| | TGTGTGGATCGCCC | | | |
| 718 | GAAAAGCTGTGTCGTGTT | P | 0.03683236 | 0.022216857 |
| | CCCTGTGAAACTGAGCA | | | |
| | GGTGTGTGTTGGCGC | | | |
| 719 | GATGAAAATGGCAAGTTC | P | 0.015751246 | 0.020770691 |
| | CCTGGCTTTCCTTCTGCT | | | |
| | CAACTTTCATGTCT | | | |
| 720 | AGGCAGAGGTTGCAGTG | P | 0.046732112 | 0.03925511 |
| | AGTCGAGATGGTGCCATT | | | |
| | GCTCTCGTTTGGGCA | | | |
| 721 | TCTTCTAGGTGATGAGTT | N | 0.034602071 | 0.00675393 |
| | TCTACTTCCTCTGGTTTTT | | | |
| | ACAACAGGAAATG | | | |
| 722 | GCTCACATGTTTACACAC | P | 0.041850185 | 0.031515402 |
| | TCAGTGCCCTAATTTCCC | | | |
| | CTGAGGGAATCGCT | | | |
| 723 | GCTGACCCAGCATCAGC | P | 0.008671832 | 0.03263513 |
| | CACACTCTGGGTTGGAAA | | | |
| | ATGTTTGCCTGTTGG | | | |
| 724 | TTACCCTGCCTGAGATTC | P | 0.047454629 | 0.001118826 |
| | CTCAGGAGAAAAGGCAA | | | |
| | CCTGCCTCCAGCCTG | | | |
| 725 | TACACGCTCACCGGCTAC | P | 0.010419999 | 0.000379399 |
| | CAGATCGACATCACCACG | | | |
| | GAGAACCAGTACCG | | | |
| 726 | CCCAGAGTCAGAGGACG | P | 0.024689974 | 0.030980339 |
| | AAGAGGAGGCTTTACCTT | | | |
| | GCACAGACTGGGAGA | | | |
| 727 | GGAAGCCAGGTGCCTTT | N | 0.022061472 | 0.0131397 |
| | AATCCACTGTAACCTCAC | | | |
| | AACTCCAAGTCCACA | | | |
| 728 | GACTGGAAGATGCAGAC | P | 0.023917358 | 0.00661157 |
| | CTTGGTTCCTGTTAGTGG | | | |
| | AAACACTGTAAGGTC | | | |
| 729 | CCTCCTTGCACCGGCCC | N | 0.00220858 | 0.001458275 |
| | TTCCTGGTCTTTGAATAA | | | |
| | AGTCTGAGTGGGCGG | | | |
| 730 | ATCATTTCCCTCTCCTAT | N | 0.005126006 | 0.00141001 |
| | CCAGAGCCCCAGCAG | | | |
| 731 | TAGGAATGAGTTCTTATC | N | 0.034267148 | 0.005906864 |
| | TAGTGTTGCAGGCCAGC | | | |
| | AAATACAGAGGTGGT | | | |
| 732 | GGTTGCACACCGGAGAG | N | 0.012260838 | 0.004125697 |
| | AAACCCTACAAATGTGGA | | | |
| | GAATGTGGCAAAGCC | | | |
| 733 | CTTGATGGGGCCTGGGT | N | 0.049931872 | 4.94551E−06 |
| | TTGTTCCTGGGGCTGGAA | | | |
| | TGCTGGGTATGCTCT | | | |
| 734 | GGTGCGGATAGTCTGAC | P | 0.049144414 | 0.032345664 |
| | TAGTTTAAGGAGACTGGC | | | |
| | CGAAGCTCTGCCCAA | | | |
| 735 | GGCCACGGCGCTTGGAA | P | 0.019159719 | 0.007783775 |
| | TCCTGGTTGTTGCTGGAT | | | |
| | GCTCTTTTGCGATTA | | | |
| 736 | CGATGGGAGGTATGTGTT | P | 0.022168071 | 0.012220665 |
| | CCCAGAACAGCCAGGAC | | | |
| | AAGAAGGTCGGTTAG | | | |
| 737 | CTGGAGAAAGTTTGCAAC | P | 0.032361446 | 0.025732387 |
| | CCCATCATCACCAAGCTG | | | |
| | TACCAGAGTGCAGG | | | |
| 738 | AGGCTCCTGTTGCTCCTT | P | 0.027511801 | 0.00431974 |
| | CACACAGACCTGGATGC | | | |
| | CCCAGAGCAAGGTCT | | | |
| 739 | CAGAACACATAGAGCTGT | P | 0.0258469 | 0.024225355 |
| | GAATGAAGATCCAGCCG | | | |
| | GCCTTGGGAGCCTGG | | | |
| 740 | CAGAGACAGCGGCTTGC | N | 0.012695134 | 0.02339725 |
| | AGTTTCCTCAAGAGGAGA | | | |
| | GAACTTAGTCCACCA | | | |
| 741 | AGGGACCAATCTGGGGC | N | 0.025320816 | 0.008456866 |
| | TGGAAATGTTAGGAGGTT | | | |
| | GCCTTGGTGCTGCCC | | | |
| 742 | AGCAATCACAAAGCCAGA | P | 0.04069892 | 0.007912336 |
| | GAAGCTGTAAGCTGCCT | | | |
| | GCCGGGCCTGAGGAG | | | |
| 743 | GGGGGGCCAGTAGTTCA | N | 0.043198983 | 0.00203468 |
| | GTGAGAATGTTTATGTTT | | | |
| | ACAACTAGCCTTCCC | | | |
| 744 | CAGTCAGCTTCAGGGCA | N | 0.039853223 | 0.006993295 |
| | GCTTCCTGCCACAGCAG | | | |
| | CATTAAATGAAGTTGG | | | |
| 745 | AGTGTTGGCAAACGAGA | N | 0.049866306 | 0.026487457 |
| | CTTTCTCCTGGCCCCTGC | | | |
| | CTGCTGGAGATCAGC | | | |
| 746 | CCGCTGAAGCTGTAGGA | N | 0.011533719 | 0.016868601 |
| | AGCGCCATTCTTCCCTGT | | | |
| | ATCTAACTGGGGCTG | | | |
| 747 | ATCCTGATGGCAAGAGCT | P | 0.031292682 | 0.000665341 |
| | ACAGCAGCGGCGGCGAA | | | |
| | GATGGTTACGTCCGT | | | |
| 748 | GTGCTCTGAGTTTCGGGT | P | 0.044136757 | 0.020429722 |
| | TCTGCTCCTACAAAGAAC | | | |
| | GTGCGGTGCTGCGG | | | |
| 749 | GGGCTAATTCTGGGAAG | N | 0.015832624 | 0.00036558 |
| | GGAGAGTTCTTTGCTGCC | | | |
| | CCTGTCTGGAAGACG | | | |
| 750 | AATATGACTATTCTAAAG | N | 0.028254109 | 0.004164029 |
| | GCTGTGAGGCCATGGGG | | | |
| | TATTGGTTAAGTTGC | | | |
| 751 | GCCTTTTTCTTCTTGAGC | N | 0.049026226 | 0.002235333 |
| | GAAGCTGTTTGAGTAAAC | | | |
| | CTGTTGAAGAGTGT | | | |
| 752 | CGGGAGGCACGGCCGA | N | 0.040972485 | 0.020278071 |
| | GATGTACACGAAGACAG | | | |
| | GAGTCAATGGAGATTCT | | | |
| 753 | GCCATGTAAGGGAGCCA | P | 0.003691716 | 0.000462364 |
| | TCTTGGAAGCAGATCCTC | | | |
| | CAGCCTCCAGTCAAG | | | |
| 754 | AGCGTTTGGTGTTACCTT | P | 0.012669954 | 0.002008159 |
| | CTCCTGGGAGGTCCTGC | | | |
| | TGCAACTCAAGTTCC | | | |
| 755 | ACAATTGCCTGGAGGCTT | P | 0.04641729 | 0.023132206 |
| | CTGTGAGATCGGGAGAG | | | |
| | GGAGGAGAGGCAGTC | | | |
| 756 | GGAGCAAGTGTTCCTGCT | P | 0.006016724 | 0.00197791 |
| | GCCAGTTCTTTCCTCTTT | | | |
| | AGGCGTGGTTGAGA | | | |
| 757 | GTAAGGTTGTCTACTCAG | N | 0.018715249 | 0.02146388 |
| | GAGGCTAAGGTGGGAGG | | | |
| | ATCACTTGAATGCCA | | | |
| 758 | GCACTCTGATTTTCTGTA | N | 0.038454737 | 0.011431254 |
| | GGTACAGTTCAAAGCCCT | | | |
| | AAAGGGAGTCTGGC | | | |
| 759 | ATCCAAAGGCCTGGAACA | N | 0.010171621 | 0.012384035 |
| | CAATGGTACTCAAAAACA | | | |
| | TAGCTGCCTCGGGG | | | |
| 760 | AGTCCCTGCCTCTCCTGC | P | 0.048684571 | 0.014032325 |
| | CTCCTCCAGTGGGGCAG | | | |
| | ACCAGAGACTCCCAT | | | |
| 761 | GTCCCTAGACCACTTTGT | P | 0.026735181 | 0.007043026 |
| | ATGACCGTTTGCAGTCTG | | | |
| | AGCAGGCCAGGGGC | | | |
| 762 | CTTGCGCAGGTGCGAGT | N | 0.031444328 | 0.022456957 |
| | GGCCAGCAGAGGTCACG | | | |
| | AATAAACTGCATCTCT | | | |
| 763 | GGAGTATGTCACCTTCCT | N | 0.013375119 | 0.01814678 |
| | GGGGGCCTTGGCTTTGA | | | |
| | TCTACAATGAAGCCC | | | |
| 764 | ATGGCCACAACATTGCCA | N | 0.040280123 | 0.03959956 |
| | GCAAATACTGCCTTGGCA | | | |
| | TCACTCAGCAGAGG | | | |
| 765 | GCCTATGATCACCTTGCA | N | 0.04484767 | 0.029280309 |
| | TGGACAGCAATCCTGTAA | | | |
| | ACATCACAGAGTGG | | | |
| 766 | GTGGCTTCTCTGTGAATT | P | 0.020299449 | 0.010809418 |
| | GCCTGTAACACATAGTGG | | | |
| | CTTCTCCGCCCTTG | | | |
| 767 | TGGAGTCTAAACGGGGC | P | 0.020393121 | 0.024710439 |
| | AGGATCTGTGCCAAAAGA | | | |
| | GCCTCTCTCCGGAGT | | | |
| 768 | GGTGCAGCTCAGTGGAA | N | 0.023709653 | 0.005153136 |
| | GATGATGACAACCAGAAG | | | |
| | ACATGAGCTAAGGGT | | | |
| 769 | TAGTGGGTTTGCTTGAGG | P | 0.009503835 | 0.008459012 |
| | TGGTAAGGCTTTGCTGGA | | | |
| | CCCTGTTGCAGGCA | | | |
| 770 | TGCCGCTTTGGACACCAA | N | 0.012436491 | 0.009907799 |
| | CACTCCCTTCTCCCAGGG | | | |
| | TCATGCAGGGATCT | | | |
| 771 | GAGAGGCCCAAGGTGGC | N | 0.024561726 | 0.003739645 |
| | CTCGCCAGCCCTGCAGT | | | |
| | ATTGATGTGCAGTATT | | | |
| 772 | TGCCCTGAGGACCAGTG | P | 0.035220257 | 0.000303077 |
| | AAAGACAGATCATAGGAG | | | |
| | AGACCCTTTTGCTGC | | | |
| 773 | GGGCCAGCAAAGAGGAG | N | 0.036389942 | 6.61113E−06 |
| | GGGTCCAGAGAACAGCT | | | |
| | GAAATACTGTCACTAG | | | |
| 774 | CTAGAGAAGTAGTGACCA | N | 0.01183771 | 0.008299415 |
| | GAACAGGGCAGAGTAGG | | | |
| | TCCCCTCCATGGCCC | | | |
| 775 | CCTCGTGTTGATTGCAGG | P | 0.02530623 | 0.009529634 |
| | AGGAGTCGGAATTAACCC | | | |
| | TCTGCTTTCCATCC | | | |
| 776 | GGAGTACGATGTGTAAGT | P | 0.032334457 | 0.012561906 |
| | GCCCATTGGGTGGCCTG | | | |
| | TTGGTCACTGTGCAG | | | |
| 777 | GTGCTCACCACCCCCAT | N | 0.039108375 | 0.044621227 |
| | GCACACGCCATCTGTGTA | | | |
| | ACTTCAGGATCTGTT | | | |
| 778 | TCACCGCTGTTGCCTGCC | N | 0.042460173 | 0.047567269 |
| | GCCTTGCGCAGTTGACTC | | | |
| | GTGTCTGAGGAAAC | | | |
| 779 | GGGAAAGTCTGAGTGAA | N | 0.044086344 | 0.011470692 |
| | AGGATGGCCTCATTCTCT | | | |
| | TTCTAATCTTGCTGG | | | |
| 780 | TCCAGAAACTGGGGATG | P | 0.034848772 | 0.049976851 |
| | GAATCTAGACTTGTGAGC | | | |
| | GGCGGTGGTGCCTGC | | | |
| 781 | CATCTGTGTGGATGTCAA | N | 0.019562795 | 0.016572468 |
| | TGACAACAACCTGGCTTA | | | |
| | CCTGGAGGCCATTC | | | |
| 782 | GACGGGGTTTTCCTTGCT | N | 0.006351665 | 0.018514432 |
| | CCTGCCAGGATTAAAAGT | | | |
| | CCATGAGTTTCTTG | | | |
| 783 | GTCATGGCCGCCTGCCT | N | 0.003787171 | 0.007482726 |
| | GTCATTCCTGACTCACCA | | | |
| | CCGTCCCCAGGTGAA | | | |
| 784 | CTGCAGCCTTTGTTACTT | P | 0.012140736 | 0.001747504 |
| | GCCCCAACGAGAAGGTC | | | |
| | GCCAAGGAGATCGCC | | | |
| 785 | GAGTGTTGCTGCTGAAG | P | 0.040163532 | 0.012613141 |
| | CACATCCTTGCAATGTGG | | | |
| | GAGTGCACAGGAGTC | | | |
| 786 | GCAAAAGCCCAAAGGTTC | N | 0.028081951 | 0.024643045 |
| | CTAAGCCTGGCTGCAAA | | | |
| | GAAGAATCAACAGGG | | | |
| 787 | GTTCTTTTGGGTCAAAGG | N | 0.010530532 | 0.003906186 |
| | AGCAGGCTAATGCAAAG | | | |
| | CTTTTGGAGACTGCT | | | |
| 788 | GGTTCCCTTCCTATTCCA | N | 0.027449757 | 0.038576162 |
| | AGCTTCCATAGCTGCTGC | | | |
| | CCTAGTTTTCTTTC | | | |
| 789 | CACGGAAAGTGCCTGCA | N | 0.013197407 | 0.015287943 |
| | AAAACAGAGGTGGATAC | | | |
| | GAGGACAGGTTGGAGC | | | |
| 790 | GCCCCACGGAACACTATT | N | 0.024911614 | 0.009952658 |
| | CCTATAAGATAGCTGAAA | | | |
| | GAAGCTGCTGTGAG | | | |
| 791 | AGGGAGGCAGCAGACTC | P | 0.013989999 | 0.026934677 |
| | AGGCCCTCCATGGTCCT | | | |
| | CTTTGTCATTTTGTTG | | | |
| 792 | CTTGGGGTTGGGGTAGG | N | 0.042843205 | 0.00245116 |
| | TAGATACCAAATGAAACA | | | |
| | CTTTCAGGACCTTCC | | | |
| 793 | CCATTTCCTGTGCCAGAA | N | 0.043176536 | 0.01756898 |
| | TCACTGCTCTATTCCATA | | | |
| | CCGTGCCATGGAGG | | | |
| 794 | AAAAGAGCTGCTGTTCCT | P | 0.017443962 | 0.002877675 |
| | AAGTAACGCGAACCCCTC | | | |
| | GCTGCTGGAGCGGC | | | |
| 795 | CCGCAACCACCTGCAAA | N | 0.025690215 | 0.003268216 |
| | CCAGAACGACTCTAGAAT | | | |
| | TTCCTTCCCCGCCCC | | | |
| 796 | CTGGGGACGAGACAGGT | N | 0.047850661 | 0.000918596 |
| | GCTAAAGTTGAACGAGCT | | | |
| | GATGGATATGAACCA | | | |
| 797 | TCCTCGGTTTCCTTGCCT | P | 0.025086836 | 0.014097738 |
| | CACCCGCGGAGAGCGCT | | | |
| | GAACCTGGACAAGCA | | | |
| 798 | CAGGCTACTTTCCGTCCA | N | 0.001323442 | 0.003324442 |
| | TGTACCTGCAAAAGTCGG | | | |
| | GGCAAGACGTGTCC | | | |
| 799 | AGTGCTGTCCCTGAAGCA | P | 0.034012703 | 0.003756254 |
| | GCTTCTGTTTCCGGCGGA | | | |
| | GGAAGACAATGGGG | | | |
| 800 | CCTGCTCTCCACAACTGT | N | 0.016670439 | 0.019570671 |
| | CCCTCCTTACCCCATGTA | | | |
| | GCTCGATCCGAAGC | | | |
| 801 | GATTGCAGGGTCCGCCT | P | 0.035011254 | 0.022838961 |
| | TCTCAAACCCCACTTCCT | | | |
| | GGACCACATCATCCA | | | |
| 802 | CCAACAAAGATGAAGTTC | N | 0.011304826 | 0.003680198 |
| | CCTATCTACGGAAAGGCA | | | |
| | TGACTGGGAGGCCC | | | |
| 803 | CAGCAGTACCTCTTTGAC | P | 0.014149474 | 0.016795126 |
| | CGCCTTGCCCACGAGATT | | | |
| | GAAGCACTGAAGTC | | | |
| 804 | GTATTTAGTGGGGAATGG | N | 0.034151123 | 0.01121221 |
| | AAAGAGTTGCCCTTGTTG | | | |
| | CAAGTAATGAAGCC | | | |
| 805 | GGCAGTTGTGCCAAGGG | N | 0.03880811 | 0.007638642 |
| | AGGAGGGAGGAGGTAAA | | | |
| | AGGCAGGGAGTTAATA | | | |
| 806 | CCAGCTAATTGGAAGGGT | N | 0.039815243 | 0.001162328 |
| | GAGGCGGGAGGATCGCT | | | |
| | TGAACCTGGGAGGCG | | | |
| 807 | GGCCCTGAAGAGGAGGG | N | 0.022026664 | 0.001204616 |
| | AGACTCTGCTGCTATAAC | | | |
| | TGAGGATGAGGCCGT | | | |
| 808 | AACTAGGATAACTTGTTG | N | 0.042843773 | 0.03912133 |
| | CTTTGTTACCCAGCCTAA | | | |
| | TTGAAGAGTGGCAG | | | |
| 809 | CCCACGACTCAGCAATAC | N | 0.032829614 | 0.035198951 |
| | CCGCCCCACCGGCTGTG | | | |
| | ATGCTCCAATAAACT | | | |
| 810 | GCCCAGCAGGCAGTCAA | N | 0.005265154 | 0.001483816 |
| | GAGAACAGCACATTAGTT | | | |
| | CCAGAAGAAAGATGG | | | |
| 811 | GAAGTCTATGATGTAAAC | N | 0.008167908 | 0.00995517 |
| | ACTTTGCCTCCTGTGTAC | | | |
| | TGTGTCATAAACGG | | | |
| 812 | GGAGGCTGAGGCGGGG | N | 0.038099237 | 0.014716403 |
| | AGATCACAAGGTCAGGA | | | |
| | GATCGAGACCAGCCTGA | | | |
| 813 | GGACCCCTGGATCCTTG | N | 0.026418502 | 0.02653536 |
| | CCATTCCCCTCAGCTAAT | | | |
| | GACGGAGTGCTCCTT | | | |
| 814 | CAGGGACAGATCTGATG | N | 0.010210457 | 0.014152462 |
| | ATCCCAGGAGGCTCTGG | | | |
| | AGGACAATCTAGGACC | | | |
| 815 | CCTGGCTCTGTCACCGC | P | 0.045672709 | 0.037065183 |
| | CATGCAACTCCATGCCTA | | | |
| | TTTACTGGAAACCTG | | | |
| 816 | GGATTGCGGATATTTACC | P | 0.009035123 | 0.007263223 |
| | TCTGTCCCCAAACGCTGA | | | |
| | CCACGCCCTGGCTG | | | |
| 817 | GGGTGTAGAGAGAATGG | N | 0.039707073 | 0.000605566 |
| | AGGGGACCAAAAAGAGT | | | |
| | CCTTCCTGGGGTGTGG | | | |
| 818 | ATCTCTGGCTGAGGGAT | P | 0.027889355 | 0.018760835 |
| | GACTTACCTGTTCAGTAC | | | |
| | TCTACAATTCCTCTG | | | |
| 819 | CCCCAGAGGGAGAGTTT | P | 0.034155899 | 0.004658483 |
| | GTGTCCATGGGTGTTATC | | | |
| | TCTGATGGCAACTCC | | | |
| 820 | CAGGGACCATGCTTCATT | P | 0.047737316 | 0.012015057 |
| | CGTGGCTGTGTCCCCAT | | | |
| | CTGAGGGCCTGGTAT | | | |
| 821 | GCTGCGTTACTGTGCTGA | N | 0.041866968 | 0.034034898 |
| | GAGGTACCCAGAAGGTT | | | |
| | CCCATGAAGGGCAGC | | | |
| 822 | CCGTCCTCCTTTCCCTGA | P | 0.029394005 | 0.005213005 |
| | GCACCTTCATCACCAGAG | | | |
| | GCTTGAAGGAACCC | | | |
| 823 | GATATGGTGCCCTGTGTA | N | 0.005289391 | 0.002880231 |
| | TCCCAGAGATGCTAGAAA | | | |
| | ACTGTTCTTTGCTC | | | |
| 824 | GAAGCTGAGGCAGGAGA | N | 0.030017106 | 0.035896652 |
| | ATGGTGTGAACCCAGTGA | | | |
| | GCCGAGATCGTGCCA | | | |
| 825 | TATGACCTTGATGCTTGG | P | 0.03575271 | 0.029689069 |
| | AGCATTCTCATTCGAGAG | | | |
| | GCACAGAATCAACC | | | |
| 826 | TGCTCGAGGCGCATCAG | N | 0.044198265 | 0.012908628 |
| | GCCCGTTTTTTACCAGTT | | | |
| | TATATCACGGTCTTC | | | |
| 827 | AGTCCAAGGAGAAGAAG | N | 0.041310503 | 0.000473461 |
| | AGGCCTAGCCTGAGCCA | | | |
| | AAGAGAGAGTACGGGC | | | |
| 828 | GTGAATGTGCCTGAGGA | P | 0.010916884 | 0.018668589 |
| | GTCTCGGAACGGAGAAA | | | |
| | CCAGTCCCCGAACCAA | | | |
| 829 | CTGCTATTAGAGCCCATC | P | 0.048607138 | 0.012326096 |
| | CTGGAGCCCCACCTCTG | | | |
| | AACCACCTCCTACCA | | | |
| 830 | GGCCAAAGCACATGCAG | N | 0.029137179 | 0.013186845 |
| | GCTCCTGGTTGTTCCTCT | | | |
| | CAAACCTGTGCTGAC | | | |
| 831 | CGGGATTGAGGAAGGTC | N | 0.012386232 | 0.010112348 |
| | CGCACAGCCTGTCTCTG | | | |
| | CTCAGTTGCAATAAAC | | | |
| 832 | CACCCCAGAAGGTTCTCT | N | 0.032636734 | 0.014138626 |
| | TGTATACCTGCTCAGTCA | | | |
| | GTTCCTTTCACTCC | | | |
| 833 | GCTAATGCCCTGGCCCA | N | 0.000590606 | 0.000716289 |
| | CAAGTATCACTAAGCTCG | | | |
| | CTTTCTTGCTGTCCA | | | |
| 834 | CGACTTCAAAGAGGGCTA | P | 0.02917516 | 0.007150569 |
| | CCTGGAGACAGTGGCGG | | | |
| | CTTATTATGAGGAGC | | | |
| 835 | TCCGCAGAGGAACTGAT | N | 0.029974336 | 0.037015486 |
| | GAGTGTGCAATTGAGAG | | | |
| | CATAGCAGTGGCAGCC | | | |
| 836 | GGCCTTTACCGGCATTGA | P | 0.043421923 | 0.025208697 |
| | TGTGGCTCATGTTTCAGG | | | |
| | CAGACTTGGGGTCC | | | |
| 837 | AGTCAGTACCGGAAGAG | N | 0.007172716 | 0.001320173 |
| | CCTGATGTGAGGGCAGG | | | |
| | GTGGGGTCTGGAACTT | | | |
| 838 | AAAGCTGCCTTCCAGGCT | P | 0.028498426 | 0.024230581 |
| | CTTGGACACTGCCTTGG | | | |
| | GAGCATCCTGCAGCT | | | |
| 839 | TGAGCCCAAGGAAGAAT | N | 0.047683302 | 0.000588721 |
| | GAGTCACCCCAGAGCCC | | | |
| | TAGAGGGTCAGATGGG | | | |
| 840 | TGTGTTGAGAGCTTCTCA | N | 0.048572176 | 0.020873162 |
| | GACTATCCACCTTTGGGT | | | |
| | CGCTTTGCTGTTCG | | | |
| 841 | TCACTGCCTTACCTCCCT | P | 0.007905519 | 8.14783E−05 |
| | CACGGTTGTTGTGAGGA | | | |
| | CTGAGTGTGTGGAAG | | | |
| 842 | GTAATCAGCTTGTTGTAG | P | 0.029274115 | 0.031032143 |
| | TGATGCTGGCCAAATGGT | | | |
| | GCTCAGCAGGTGAG | | | |
| 843 | CTTCCATCTGGCTGCACT | N | 0.028841562 | 0.01119674 |
| | CCAAGGCCCCCTCTGTC | | | |
| | CTTTTCAGAACACAT | | | |
| 844 | TCCCTCCCTTACCCCCAT | N | 0.042282802 | 0.011360043 |
| | CCTTAACTTTGTATCCTG | | | |
| | GCTTATAACAGGCC | | | |
| 845 | TGGCCTTTGGGAAAGATC | N | 0.027599427 | 0.011795299 |
| | AGAGAGGCAGAGGTGGC | | | |
| | ACAGGACAGTAAAGG | | | |
| 846 | GATGCCCCTGACATCATC | N | 0.036143648 | 0.045925012 |
| | ATTCTTGTGGGAGACAGC | | | |
| | AGCCTGTATGTGGT | | | |
| 847 | CAATGAGAAGCCACAGG | P | 0.003955926 | 0.00043235 |
| | TGATCGCGGACTATGAGA | | | |
| | GCGGACGGGCCATAC | | | |
| 848 | CCCTTGGCCTGCCACTTT | P | 0.024191337 | 0.012090949 |
| | CCAGGTGTCCTTTATCAC | | | |
| | TTTGACGGGACTCT | | | |
| 849 | ACCAAGATCGCGGCGCT | N | 0.023301321 | 0.008267617 |
| | GCACTCTAGCCTGGGTG | | | |
| | ACAGAGTCAGACTCCG | | | |
| 850 | AGAGGAGTGGTTTGTGA | N | 0.013545538 | 0.025236758 |
| | CAAGCGGAATCCAAATG | | | |
| | GCATTCGAGTGGCTCC | | | |
| 851 | CCTTCAGGAGACTTGATC | P | 0.02178259 | 0.013947278 |
| | CCAGTAGACTGAGGTCTT | | | |
| | CCCTTTCAGCAGAA | | | |
| 852 | TGCTATGGGGCTTAGGC | N | 0.037494378 | 0.000185732 |
| | CATGCTCAGTGCTGGGG | | | |
| | ACAGGAGTTTTGCCCA | | | |
| 853 | GGTAGAGATGGGCGGGT | N | 0.019826375 | 0.00035875 |
| | TTGTGCTATGTGCAGGGT | | | |
| | GGAAGGGAGGGAAGT | | | |
| 854 | CTCCCCATCCCAACCAGA | P | 0.033817525 | 0.000227676 |
| | GATGGCTCACTTCGGATC | | | |
| | GAGGGTTGACTACA | | | |
| 855 | CGCTCTTTCTAGTGCAAG | N | 0.03397391 | 0.015933777 |
| | AAACTGCAGGCTGGATCA | | | |
| | GTAGTTCAACAGCT | | | |
| 856 | TCTGGGAGCTGGGATGA | N | 0.022801912 | 0.007160103 |
| | TAAAGATGAGGCTTGCG | | | |
| | GCTGTGGCCCGCTGGT | | | |
| 857 | TAGGAGGATCGCTTGAG | P | 0.02266909 | 0.014980681 |
| | CCTGGGTAGAGGCTGCT | | | |
| | GTGAGCTGAAATGGCG | | | |
| 858 | ACTTGAGCAAGACTGATA | N | 0.044949264 | 0.019344398 |
| | CCACCTGCGTGTCCCTTC | | | |
| | CTCCCCGAGTCAGG | | | |
| 859 | AGTCCTGGCCTCCGCAG | N | 0.026590931 | 0.025600931 |
| | ATGCTTCATTTTGACCCT | | | |
| | TGGCTGCAGTGGAAG | | | |
| 860 | GGGAAGTCTGAGCGAGT | P | 0.040004869 | 0.037373453 |
| | CTCCTAAATACTCTGGGC | | | |
| | TTTAGCTTCTCCAGC | | | |
| 861 | CTGGCATGGTTTGCGGA | N | 0.039245771 | 0.00549941 |
| | GGTTAGATTTACTGGAAA | | | |
| | TGTATTCATACTGTG | | | |
| 862 | GCACCGGGTGGCAGATG | N | 0.042456323 | 0.010691492 |
| | TTCTATGCAGTGTGGTTC | | | |
| | AAGTTTCTTTGACCG | | | |
| 863 | AGCAGCGACCGAGACCC | N | 0.00857263 | 0.006048358 |
| | GGTGGGACACTCCCCTT | | | |
| | CTCCCCACTTTCACCT | | | |
| 864 | TCTGATCTTGAGTCTGGA | P | 0.018214521 | 0.002465285 |
| | ACTGACAAGTTGTGTGAC | | | |
| | CCTCAGCAAGTCAC | | | |
| 865 | AGTGCTGCCCTCTGGGG | N | 0.012925546 | 0.005394539 |
| | ACATGCGGAGTGGGGGT | | | |
| | CTTATCCCTGTGCTGA | | | |
| 866 | GGGCCCCTGGGCAGTGG | N | 0.033484652 | 0.010538807 |
| | GTTTTGGGCAAATTCCCT | | | |
| | TTCTTTGCATCCACA | | | |
| 867 | GAAATTCTGCCTGAGGAC | N | 0.04280588 | 0.001361253 |
| | AGCAGCCCAGTGCTTGG | | | |
| | CGAGAGTTCCTGACA | | | |
| 868 | GCGCTGCCTTTCTTCAGC | P | 0.025676175 | 0.00031795 |
| | AACAGACCCTCAAACCAA | | | |
| | GAGGAAGCTAGATG | | | |
| 869 | CCCTGGGGGAGAGACTA | N | 0.041302023 | 0.023645188 |
| | GAAAACACAGAAGGAAG | | | |
| | CAGCACAGGGAGACCC | | | |
| 870 | GGATTTTCACTGTTCTAA | P | 0.020557499 | 0.012563284 |
| | GCTCCCCGTGACCTGTG | | | |
| | GTGAGGCGAAAGGGA | | | |
| 871 | TGTCCTGTGACTGCCCCA | N | 0.014536437 | 0.001596642 |
| | CAGAGATAAGGGGCCAG | | | |
| | GAGGGATTGAAAGGC | | | |
| 872 | TGCTGTAGCGTGGATAG | P | 0.043733371 | 0.000485558 |
| | CTGTGATTGGTGAGTCAA | | | |
| | CCGTCTGTGGCTACC | | | |
| 873 | GTGCAAACAGACATTCCA | N | 0.015987559 | 0.019668438 |
| | GAGAGCCTGATCCACATC | | | |
| | CAGCAGCAGAGCCC | | | |
| 874 | TTCACTGACTGCGGCATG | P | 0.013164047 | 0.005274099 |
| | TCCTCGTGTCTTCTCTGA | | | |
| | TTTTGTGGTACATG | | | |
| 875 | ATGAAGAGGGACAGCGA | P | 0.017327441 | 0.000675956 |
| | TTCCGAGGGTGACTGAG | | | |
| | GCTACAGCTTCTATCA | | | |
| 876 | CCTGTGGTAAGCAACCTG | N | 0.010120249 | 0.030845743 |
| | GGCATCTTAGGAAGCAGT | | | |
| | CCCTGGAGAAGGCA | | | |
| 877 | CATCAGTCCCAACAAGAT | N | 0.024961523 | 0.029863546 |
| | GGCCTAGAAATCGCATTC | | | |
| | TCACCTCGCCTTGC | | | |
| 878 | TCTCTCCAGAGTTGCATG | N | 0.02280555 | 0.012293663 |
| | TAGATAGCATTTATTTCTG | | | |
| | TGCCCTTAAACCC | | | |
| 879 | CCAGCCCAAATGTAGTCT | P | 0.016997695 | 0.004433253 |
| | GCCTTGAAAAGTCTTTCA | | | |
| | GCTGTGACTGCAGG | | | |
| 880 | TAGTATATGCCCTAAAAC | P | 0.008860115 | 0.005066752 |
| | GTCAGGGGCGCACTCTG | | | |
| | TATGGCTTCGGCGGC | | | |
| 881 | CTCACCCCTAGACGTTGC | N | 0.036282748 | 0.003518607 |
| | CAACCAGAACTGACGTGT | | | |
| | GACCTCCTGGGTGT | | | |
| 882 | GTTCGCTCAAAATACTCA | N | 0.01386742 | 2.03851E−05 |
| | ACAGGGGAATAGGCAGC | | | |
| | GGACAGTCAGAATGG | | | |
| 883 | AGAACAGATGGCTGTGAA | N | 0.019638256 | 0.003774624 |
| | AATTACACCCATGCACAG | | | |
| | AACAAGCCACAGGA | | | |
| 884 | CGATGAGAAGGTTTACTA | N | 0.040313587 | 0.038737835 |
| | CACTGCAGGCTACAACA | | | |
| | GTCCTGTCAAATTGC | | | |
| 885 | CACAGTGGAGAAGACCC | N | 0.034984627 | 0.028433625 |
| | TGCATCAGGGAATGTGG | | | |
| | AAATGACTTTGCTGAA | | | |
| 886 | CTCTTTGGCTTAAAATGT | N | 0.019885992 | 0.002207904 |
| | TCAGCAGAATTGGGCAGT | | | |
| | GGGGGTGACTTTTC | | | |
| 887 | CGCTTCGTACCCAGCCA | N | 0.017609595 | 0.016017299 |
| | GCACTATGTGTACATGTT | | | |
| | CCTGGTGAAATGGCA | | | |
| 888 | ACTCTAGGTTTCTACTTG | N | 0.02408877 | 0.003818878 |
| | ATTTTTCCCCCATGTATA | | | |
| | CCTTTCATCTGTTC | | | |
| 889 | GCTATTAATTTCCATCCTT | N | 0.039578239 | 0.048732936 |
| | TAGCAGGCTGGGCCCTA | | | |
| | GGCAGGAAGCTGGC | | | |
| 890 | GGACGAAGAAGATTACG | P | 0.013639774 | 0.002190215 |
| | ACTCCTAGCGCCTTCTGC | | | |
| | CCCCCAGACCATAGC | | | |
| 891 | GGGCCAAGAAAGCAACT | P | 0.04844356 | 0.021975351 |
| | TGAGCCTTGGGCTAATCT | | | |
| | GGCTGAGTAGTCAGT | | | |
| 892 | GCAGAGAGGATTTTCTGT | N | 0.029902089 | 0.001160177 |
| | CATGGAAAAAGCTTGGG | | | |
| | GCATTCGTTTTGCTC | | | |
| 893 | GCTGCTGTAACAGGGAC | N | 0.007616866 | 0.010017932 |
| | TAGCACAGACACACGGAT | | | |
| | GAGTGGGGTCATTTC | | | |
| 894 | CTCACCCAACAGATCTTT | P | 0.025291125 | 0.015883315 |
| | CCAGAGGTCCATGGTGG | | | |
| | AAGACGATAACCCTG | | | |
| 895 | GGTCTGGTAGCCAACAA | N | 0.046730735 | 0.034653685 |
| | CTTGGCAACTTCCACTCC | | | |
| | TTCTCACCTCGTGAG | | | |
| 896 | GAGCATTTTTTCATGTGC | N | 0.018285113 | 0.011366685 |
| | CAGAGCCTGTACTGGAG | | | |
| | GCCCCCATTGTGCAC | | | |
| 897 | ACCAGGTGCAGACACGC | P | 0.002861999 | 0.001024792 |
| | AGTGCTGATGAGCCCAT | | | |
| | GACTACCTTTGTCTTA | | | |
| 898 | TCTGCCAACCCCACCAGT | P | 0.031227636 | 0.033115566 |
| | GATGAAAATTAACTGTGG | | | |
| | GCCACTCGCTGCAG | | | |
| 899 | GTGCTCTTTGTTCATCAT | N | 0.006169618 | 0.007467555 |
| | TGGCCCTCATTCCAAGCA | | | |
| | CTTTACGCTGTCTG | | | |
| 900 | CCTGTGTCTTGTGCCTGG | P | 0.000692635 | 0.003296845 |
| | GAGAGTGGGATGAAACG | | | |
| | CCACAGAGCAGCCTG | | | |
| 901 | CCTCAGCCATGGAGACC | N | 0.032624981 | 0.010520608 |
| | ATGTCATGCAGAATTAAC | | | |
| | AAGGTAGCACCGAGC | | | |
| 902 | GGATCCTTCCTGCAGTG | P | 0.037477009 | 0.008674557 |
| | GCTCGGGTGAGATGGTA | | | |
| | TGCCGAGCAGACAGCT | | | |
| 903 | AGGGCTGCAGGGCCTCC | P | 0.041918417 | 0.028533727 |
| | CACCTTCCAACAGACAGG | | | |
| | CTCTGCTGTATCTGT | | | |
| 904 | TGGGGGATTTTTCAGTGG | N | 0.037770679 | 0.026011238 |
| | AACCCTTGCCCCCAAATG | | | |
| | TCGACCAGCCCCCA | | | |
| 905 | GGTGCAGCTCCTCAGCG | N | 0.006296763 | 0.004801343 |
| | GCTCCAGCTCATATGCTG | | | |
| | ACAGCTCTTCACAGT | | | |
| 906 | GAACTGTCAAGGGAGGT | N | 0.036673266 | 0.017594555 |
| | GCTGGAGAGGGATTAAC | | | |
| | CTGTGCTGCCTGGGAC | | | |
| 907 | GCCCGCACCCACCATGG | P | 0.037946296 | 0.002266569 |
| | CCACAGTTCAGCAGCTG | | | |
| | GAAGGAAGATGGCGCC | | | |
| 908 | ACAGCCCTCAGCGAGAG | P | 0.038720375 | 0.004336818 |
| | TGCCAACCAGGCCTTCCT | | | |
| | GGGCTTCACATACGT | | | |
| 909 | AGGAATTCATCTTCAGTC | N | 0.010753369 | 0.006934658 |
| | TACCAGCCCCCGCTGTG | | | |
| | TCGGATACACACTCG | | | |
| 910 | TCTCTGGAATTGTGGAAG | N | 0.034620304 | 0.017278882 |
| | TGGTTGGAAGAGTAACC | | | |
| | GCCAAGGCCACCATC | | | |
| 911 | CTCCTTTCCCCACAGCAG | N | 0.016336887 | 0.006998385 |
| | CTTTCTTTCCTGTCAACTA | | | |
| | GAAAGGAGCAGGA | | | |
| 912 | AGCTGACCAGATCCCAG | P | 0.003880796 | 0.003404287 |
| | GTGTTCCAGAGCGAGTTC | | | |
| | TTCAGCGGACTCATG | | | |
| 913 | ACGGCGTTCCTGGCTCT | N | 0.005571444 | 0.005861409 |
| | CCTGCCCACAGGATGAA | | | |
| | CATTTTCGGCTTCCTT | | | |
| 914 | TGTGGGTGTAGATGGGG | N | 0.03596499 | 2.79435E−05 |
| | TTCGAGCTTTCCTACAGA | | | |
| | CCTATTCTCAGGAGT | | | |
| 915 | CACGCCTCCGTGGGCTC | N | 0.005382881 | 0.002722711 |
| | CATTCTGTAGCATTGCCA | | | |
| | GCGTTCTCTTCACGT | | | |
| 916 | GACCATCCCAAAAAGGAA | N | 0.011972906 | 0.000135103 |
| | GTGCACCTTGGAGCCTG | | | |
| | TGGAGCTCTCAAGAA | | | |
| 917 | CGGAGAAGACGAGGAAG | P | 0.015850098 | 0.001624045 |
| | AGGAGGAGCAGTTGGTT | | | |
| | CTGGTGGAATTATCAG | | | |
| 918 | TGTGATTCCAGCCACCGC | N | 0.034925394 | 0.045413684 |
| | CTGACCATCCGCCATTCC | | | |
| | GACTGCTAAAAGCG | | | |
| 919 | GTACACGGACTGAATCTG | N | 0.020710609 | 0.020040368 |
| | CACAGAGCAAGATGCTG | | | |
| | AGTGGAGTCGGGGGC | | | |
| 920 | CGCTGTTTCGTCAAAGCA | N | 0.01130778 | 0.00224005 |
| | CGAGGGCCGCCTGTGGC | | | |
| | CTTAATTCCTAACGG | | | |
| 921 | GAATCCTTAGGCGTGGTT | P | 0.019426966 | 0.000212645 |
| | GTGGCCGTCTTGGTCAC | | | |
| | CTGTGTGCCACTTGC | | | |
| 922 | GGGCCGAGACCCAGACG | N | 0.031733655 | 0.011597688 |
| | AGGAGTGAGGAATGAGA | | | |
| | GAGACCAAAGTTCCTG | | | |
| 923 | GAATCGTGATGTCCAATA | P | 0.010891107 | 0.008327588 |
| | CAGAGGCAGAGTCCGGG | | | |
| | TCCAGCTCAAACAGG | | | |
| 924 | CACGGACATGGGGCAAG | N | 0.014365736 | 0.000296075 |
| | CCAGGGCCCAGAGCCTT | | | |
| | TGGCTGTACAGAGACT | | | |
| 925 | GCTGAACAAGGCGGCAT | N | 0.039738483 | 0.020462205 |
| | GCACATGCTACTCCAGAC | | | |
| | GCTGAAGTGGGAAGA | | | |
| 926 | GGGCCATCATCCTCTTCA | N | 0.004941223 | 0.002725446 |
| | TCATCCTCTTCATCCTGC | | | |
| | TGCTGTTCCTGGCC | | | |
| 927 | GACACTTGCACAGCATG | N | 0.012321686 | 0.030362709 |
| | GCTCTGCCTCACAATGAT | | | |
| | GCAGTCAGCCACCTG | | | |
| 928 | ACGCCAGCTGGGCGTCA | N | 0.00747404 | 0.011171192 |
| | GACCCCACCGGGGCAAC | | | |
| | CTTGCAGAGGACGACC | | | |
| 929 | CTGTGCCAGGACTGTGTT | N | 0.013492037 | 0.027035952 |
| | TTTAGCCCTTCACCTCTC | | | |
| | AGCTTTAGCAGGAC | | | |
| 930 | TGGACTGTGCCACACAC | P | 0.043660171 | 0.043426569 |
| | CCAGTGATCCATCCAGAA | | | |
| | ACAAGGACTGCAGCC | | | |
| 931 | TCTCACGCTGATGGCTTG | P | 0.042477216 | 0.004328097 |
| | GCAGAGCACCTTCGGTTA | | | |
| | ACTTGCATCTCCAG | | | |
| 932 | TTGGCTGCCGTCTGGCC | N | 0.031528303 | 0.014147322 |
| | GGGCTCTCATGGTACTTC | | | |
| | CTCTGTGAACTGTGT | | | |
| 933 | GCTGGAGTATATCCTGAC | N | 0.046688854 | 0.005319417 |
| | CCAGACCTACGACATTGA | | | |
| | AGATTTGCAGCCGG | | | |
| 934 | GGTGCTAAGCCCTCTCTC | P | 0.013768335 | 0.000441332 |
| | CACAATGCCAAGACGGA | | | |
| | GACCACAGCCTACAC | | | |
| 935 | GGGCTGCCCCTCCCCCT | P | 0.042018362 | 0.039643108 |
| | GCACAGTAGTTTGTCCTG | | | |
| | TGGTTTATTTTGTAT | | | |
| 936 | CAGTTTTGCCTCCAGTGG | N | 0.041220957 | 0.030446149 |
| | AAGCAGAAAGGGTTTTTT | | | |
| | CAGCTGTTAAATCC | | | |
| 937 | TATACACTTCTGGCTCAC | N | 0.022609471 | 0.010260427 |
| | AGGAAAGTGTCTGCAGTA | | | |
| | GGGGACCCAGAGTC | | | |
| 938 | GCATGCCAAGCAGCATG | P | 0.04194826 | 0.010699579 |
| | CTCAGTGGGGTTTTAGGC | | | |
| | TGTCACATGCAGCTG | | | |
| 939 | GGGCTTAACTTTACCTCC | N | 0.045065213 | 0.002960463 |
| | TTGAAAATGACATGGACT | | | |
| | TGGGTGGATGGTTC | | | |
| 940 | CACTGCATTTGGGCACCA | P | 0.048587397 | 0.045924989 |
| | TCTCAGCTCCCTTGCATC | | | |
| | CAGGTGCAGCATGG | | | |
| 941 | CCTGTGGTAGTGCTCCCA | P | 0.020828888 | 0.00670402 |
| | GTCTGACCTCTGTAGACC | | | |
| | TTCAGTACTCACTC | | | |
| 942 | ACTGTAGAGGAGCCCCT | P | 0.033557961 | 0.03705894 |
| | GGATCTTATCAGGCTCAG | | | |
| | CCTAGATGAGCGAAT | | | |
| 943 | GCTCTTGGAGGAACGAG | N | 0.027462016 | 0.010188812 |
| | CAAAACAGAAGCGGTGC | | | |
| | ATACCTCAGAGCCTGG | | | |
| 944 | TGAAGGATGCCAAGAATG | N | 0.027069273 | 0.008634204 |
| | AGAAAAAGCAAGGGGTTT | | | |
| | GTCCAGGTGGCCCC | | | |
| 945 | GGCCTGGCTTCTGGGCT | N | 0.028348338 | 0.023469084 |
| | GATGGGTCAGTTGGGCC | | | |
| | TTCATAAACACTCACC | | | |
| 946 | GCATGCATGGGAGGAAT | N | 0.026025811 | 0.019766904 |
| | TCATCTTCAGTCTACCAG | | | |
| | CCCCCGCTGTGTCGG | | | |
| 947 | GACTTCTGTTTGATGGCT | N | 0.037119518 | 0.00723518 |
| | TACAGCAACCAGTACTCA | | | |
| | ACAAGCAGCTGACT | | | |
| 948 | AGCTCACATGCAGTAGAC | N | 0.027029627 | 0.000170759 |
| | TTGGGCAGGCAAAGGGG | | | |
| | GCACCAAGGGCACAG | | | |
| 949 | GACTCTGGAGGCGCGAA | N | 0.048065742 | 0.000910947 |
| | TCAATAGAGCCACGAACC | | | |
| | CCCTGAACAAGGAGC | | | |
| 950 | CGGCATCATCCCATCTCT | P | 0.031448195 | 0.000405012 |
| | AATTTCCCCTCTGTCCTC | | | |
| | CATCCAGCGGCTTC | | | |
| 951 | CACTAATGCCAGGCTGAC | P | 0.030641996 | 0.003857552 |
| | GTGTCTTGGAGTGTGGCT | | | |
| | AGACAAAGTGGCAG | | | |
| 952 | GTTTCTCTTTGATGACCA | N | 0.04106078 | 0.043701727 |
| | GGAAGAAATCCCAGCAC | | | |
| | CCCAGCCACAGGCTG | | | |
| 953 | TGCTGAGAACAAACCCAC | N | 0.010110761 | 0.013252603 |
| | CTGAGCACCCCAGACAC | | | |
| | CTTCCTCAACCCAGG | | | |
| 954 | GCACTAGTTAGACTCTTT | N | 0.021722118 | 0.009267607 |
| | AGAATACTCCAAGAGTTA | | | |
| | GGGCAGCAGAGTGG | | | |
| 955 | TTTGCAAAGGGCCAAATT | N | 0.002820511 | 0.001666937 |
| | TCCCCAAACTGAACGGG | | | |
| | CTCAGGAAATGTTCC | | | |
| 956 | TGGGAAAGTGTGAGTTAA | N | 0.026632411 | 0.00172759 |
| | TATTGGACACATTTTATC | | | |
| | CTGATCCACAGTGG | | | |
| 957 | GCGGGAAAATGGGAAGT | P | 0.038032656 | 0.01438077 |
| | CCCTCTGCCTAAAGTACG | | | |
| | TGCCCAGGGAGAAAC | | | |
| 958 | GGAGTTGTTTTCGGCAG | N | 0.006793701 | 0.000320843 |
| | GGGCCTTGTCTCTCACTG | | | |
| | CATTTGGTCAGGGGG | | | |
| 959 | TCTGCTGCTCTGGATGGC | P | 0.029544576 | 0.016662422 |
| | TGAAGGCTCCTGGGCCA | | | |
| | TCTTCATGTGCTGCT | | | |
| 960 | GCTCTGTATTAGAAAGCC | P | 0.011441511 | 0.038042021 |
| | CCTCAGAACTGGGAAGG | | | |
| | CCAGGTAACTCTAGT | | | |
| 961 | CTGCATGCTGGGTTCTGC | N | 0.042641217 | 0.036195401 |
| | ACAGCTGGCCTCCCGCG | | | |
| | TTGGGCAACATTGCT | | | |
| 962 | CTTGAGCCCATTGAAACT | N | 0.028074176 | 0.038617203 |
| | GATCTTGAGCTCCTGGCC | | | |
| | TCCAGAATTGCAGG | | | |
| 963 | CCAGGGCAGCATGTGAT | P | 0.039886125 | 0.024997349 |
| | TCATTTGGGGATGGAAG | | | |
| | GAATCTGTCCCGCATC | | | |
| 964 | CCTGGCAACCAGTGGGA | N | 0.018132036 | 0.00014155 |
| | AAAGAAACATGCGAGGCT | | | |
| | GTAGGAAGAGGGAAG | | | |
| 965 | TTGTGCCACTGCACACTC | P | 0.006183295 | 0.004821666 |
| | CAATCTGGGTGAAAGACC | | | |
| | GAGACTCCGCCTCA | | | |
| 966 | ACTCAAAGCTAAGGAGCA | P | 0.040367681 | 0.015697891 |
| | GTCAGGAACCCAGATAA | | | |
| | GAAAGCCATCCTAGT | | | |
| 967 | AAGCAGGTCAGCAACATC | N | 0.028710755 | 0.001207445 |
| | GGTCGCAAGGACTACCT | | | |
| | GGCCCACAGCTCCAT | | | |
| 968 | CAGTGAGCCGTCATCGC | N | 0.014674459 | 0.004849601 |
| | GCCACTAAGCCAAGATC | | | |
| | GCGCCACTGCACTCCA | | | |
| 969 | TCCAGTGGGCCCTCGGT | P | 0.022442696 | 0.000733978 |
| | GCCTGCTGTGAACTGCTT | | | |
| | TCCCTCGGAATGTTT | | | |
| 970 | CAGTGAAGCTGACAAAAT | P | 0.015762306 | 0.001668159 |
| | CAAAGTGGCCCAGGGAG | | | |
| | TGTCTGGTGCTGTGC | | | |
| 971 | GTTCGTTTCATCAGGCTC | P | 0.031640521 | 0.002964041 |
| | TGTTCCTCAATGGCCTTT | | | |
| | TGCTACGTGCCTCC | | | |
| 972 | CAGGGACCTGAGAGTAA | P | 0.019919102 | 0.007317436 |
| | GCACATGACAGCGTCTG | | | |
| | CTTGCGTTGTGTCTGT | | | |
| 973 | GATGATCCTACTGCTCCC | P | 0.025290991 | 0.006065518 |
| | AGCAACCTCTACATCCAG | | | |
| | GAATGAGTCCCTAG | | | |
| 974 | GATTCTCCCCGGACTCTC | P | 0.043150649 | 0.005204634 |
| | CGTCATGGGCGTGTGCT | | | |
| | TGTTGATTCCAGGAC | | | |
| 975 | CTAATTCAGAGGTCTCAT | P | 0.043645757 | 0.007879374 |
| | GCCTGCCCTTGCCCAGA | | | |
| | TGCCCAGGGTCGTGC | | | |
| 976 | GAAAAGGTGTTTGTGCTC | N | 0.026600349 | 0.000886913 |
| | CGTTTTGTTTCTGCTCAG | | | |
| | TAATATAGTCAAGC | | | |
| 977 | ACGGGTCCAGTGTGTGC | P | 0.030101967 | 0.00543662 |
| | TTGGCGTGTTTTCAGGGA | | | |
| | GGCAGAGAAAGGCTC | | | |
| 978 | TTTAAGGACTGCTGATGC | N | 0.007121695 | 0.002736018 |
| | CCCCTCAGGCCTCCCCC | | | |
| | AAGTTTGCTGGGCTT | | | |
| 979 | CCAGGTACTAGGGAGGC | N | 0.035228862 | 0.006746677 |
| | TTTTGAACCCAGGAGGCA | | | |
| | GAGGTTGCAGCGAGC | | | |
| 980 | CAGTTCCCACAGGGCAT | P | 0.011374124 | 0.026387276 |
| | GTGACTTTGAAAGAGACT | | | |
| | AGAGGCCACACTCAG | | | |
| 981 | GCCCAGGAGACAGACCA | N | 0.032154728 | 0.006946941 |
| | CTTGCCACGCTGTTGTAA | | | |
| | AAACCCAAGTCCCTG | | | |
| 982 | AAGGACGTCACGGGCCC | N | 0.04462393 | 0.015818302 |
| | CTCTAAAGGATTCGTGGT | | | |
| | GCTCATCCCCAAGCT | | | |
| 983 | TGTAGGGTTGAGCCACA | P | 0.041129768 | 0.006420567 |
| | GACAGCTCTTCAGCCCA | | | |
| | GTAGCAGTGGAGCAGG | | | |
| 984 | GCATTGCTGAGAACAAAC | N | 0.024155195 | 0.031464057 |
| | CCACCTGAGCACCCCAG | | | |
| | ACACCTTCCTCAACC | | | |
| 985 | GTAGGGGTTTCCAGCTTC | P | 0.02324507 | 0.006830172 |
| | CCCAGGCTCCGGCCTTG | | | |
| | TCAGTCTCTTTGCAT | | | |
| 986 | GAGGGACGCTATGCCTC | N | 0.016013033 | 0.013273944 |
| | ATGCCCGTTTTGGGTGTC | | | |
| | CTCACCAGCAAGGCT | | | |
| 987 | AGGTGTGATTGCGCCACT | N | 0.035006357 | 0.011413734 |
| | GCATTCCAGCTTGGGCG | | | |
| | ACAGAGCGAGACCCA | | | |
| 988 | CATGCCTCTGTGCCTTCG | N | 0.009855212 | 0.017387359 |
| | CTCATGCTGTTTCTTCCG | | | |
| | ACTGGAATGCCTTC | | | |
| 989 | GATGACACCTTTGAGGCC | P | 0.045584996 | 0.018950627 |
| | CTGTGCATCGAGCCGTTT | | | |
| | TCCAGCCCGCCAGA | | | |
| 990 | CTTGGCCCGAGCCCCTC | N | 0.011899783 | 0.003284773 |
| | CGTGAGGAACACAATCTC | | | |
| | AATCGTTGCTGAATC | | | |
| 991 | TGGGGCAGCAGTTGGGG | N | 0.041027677 | 0.000546288 |
| | AAGTGTCTGCTGAGAATA | | | |
| | TCAAGGGGAAGAAGC | | | |
| 992 | GTGCATCACACTTAACTC | P | 0.032283297 | 0.001907383 |
| | ATCTAACTGCTTCCCCGG | | | |
| | ACACCCTCCACCTC | | | |
| 993 | TCACACTGGCGCTAAGC | P | 0.026547392 | 0.010405859 |
| | CCTACAAGTGTCAGGACT | | | |
| | GTGGAAAAGCCTTCC | | | |
| 994 | ACGGTCAGGGTCTTCTTG | N | 0.00173334 | 0.004520524 |
| | CGACCCGGCCCGCTCCA | | | |
| | GATCCCCACAGCTCT | | | |
| 995 | GCCCTCCCTCAATTCCCC | N | 0.019745264 | 0.048802485 |
| | TGTAACATTCCTGAAGCT | | | |
| | GTTCCCACTCCCAG | | | |
| 996 | GCTCGAGATATGAGTTCT | N | 0.039396138 | 0.042365573 |
| | GCAAAAGGTGGTCCGCA | | | |
| | TCCTTGGCCCTCTGG | | | |
| 997 | AGAAACCTCTGGGAGACT | N | 0.010262207 | 0.000442073 |
| | GGAAACCTGATTGGAGC | | | |
| | ACTGAGGAACAAGGG | | | |
| 998 | TCTCTGCAGCACGATTTC | N | 0.043448304 | 0.003918278 |
| | TCTTTTGATAATGCCCTTT | | | |
| | AGGGCACAACTAG | | | |
| 999 | AATGCTGCAGTTCCTGAT | P | 0.016318933 | 0.00122668 |
| | GAGATCCCCCCTCTCGA | | | |
| | GGGCGATGAGGATGC | | | |
| 1000 | CCTGATGTGGAAAGCAG | N | 0.022532293 | 0.014238884 |
| | GGGTTTCTGGTCTACTGG | | | |
| | CTAGAGCTAAGGAAG | | | |
| 1001 | ATAACTCGTCTGTCCAGG | N | 0.015485874 | 0.006106456 |
| | GTTTTGGTGGCTGGCGA | | | |
| | AGCTGAGGGCGTGTC | | | |
| 1002 | GACCCTGTGGAGAAATTC | N | 0.0492634 | 0.00546858 |
| | GTTGTTCCCACTGAAATG | | | |
| | GACTGACTGTAACG | | | |
| 1003 | CTATCTGGGGGAATAGAA | N | 0.020224544 | 0.000349189 |
| | AGCCCACAGTCTTCTGAG | | | |
| | TTGTGCTACACCAA | | | |
| 1004 | CTTGGTGCTGATGGCCT | N | 0.031848209 | 0.000296378 |
| | GAAGGGGCCTGAGCTGT | | | |
| | GGGCAGATGCAGTTTT | | | |
| 1005 | GTTGTGTCTGGAGAAGAA | N | 0.048323313 | 0.02965325 |
| | GCTGGGTCAGGGGTGTT | | | |
| | TCGCTGAAGTGTGGC | | | |
| 1006 | AGGCAGGCCTCATTTCAT | N | 0.029296873 | 0.045010304 |
| | CACGCAGCATGTGCAGG | | | |
| | CCTGGAAGAGCAAAG | | | |
| 1007 | CCCTGCACCTGGTCCTG | P | 0.002688107 | 0.000250415 |
| | CGCCTGAGGGGTGGCTG | | | |
| | TTAATTCTTCAGTCAT | | | |
| 1008 | TCTGTGCCGCGGAAACC | P | 0.033515535 | 0.007802703 |
| | GATGTGGAAGACCCCGA | | | |
| | GGTGGAGTGTGGCTGA | | | |
| 1009 | GAGTGCCTCCGGCCTCC | N | 0.017487111 | 0.003872454 |
| | CCATCCACCTCTGCCTAA | | | |
| | GTAAATCTGCTCTCA | | | |
| 1010 | TGACCCCAGCACACCTCT | N | 0.028604961 | 0.029255316 |
| | GGCTAACCCATACCCCAC | | | |
| | ACCTGCCCAGCTCT | | | |
| 1011 | CTTGAGAGATGAGCACCA | P | 0.019566551 | 0.000613113 |
| | GTTACACAAGGACTTCTT | | | |
| | TATCCGAGCGCTGG | | | |
| 1012 | GCGTAGTCAGCCCTTGC | N | 0.014976383 | 0.016759227 |
| | GCTCAGTGTAGAAACCCA | | | |
| | CGTCTGTAAGGTCGG | | | |
| 1013 | GGGGGGCTGGCGCCAC | N | 0.027227473 | 0.009130125 |
| | CGAACCTGCACATCTCAA | | | |
| | CTTGTAACTCAATAAA | | | |
| 1014 | GCCCAGCCAGCACGCCT | P | 0.004597134 | 0.000218954 |
| | CAAGGTAGATGGAATCCC | | | |
| | CACTGGTCAGAGAAA | | | |
| 1015 | GAATCCCTATGTATGAGA | P | 0.009152623 | 0.001434882 |
| | GGAGGGAGGCAGGCTGC | | | |
| | AGCTTCAGCCACAGA | | | |
| 1016 | GCAGCTATTGAAAGGTTA | P | 0.049606162 | 0.024172653 |
| | CTGGGCTCCCAGCCATC | | | |
| | ATAGCAGCATTTCTG | | | |
| 1017 | TGGCCCCAATACCCATTT | N | 0.025466104 | 0.000592227 |
| | TGGAAGCCCCTGTGGCC | | | |
| | GTGTGGATGTCGGTA | | | |
| 1018 | GCTTCTCCTATGAGGTGC | P | 0.039537068 | 0.003530351 |
| | TTAGGGCTGCATCTTGGT | | | |
| | TTTAGGACCACTGT | | | |
| 1019 | GACTGGCAAGGTTTCCTA | N | 0.038431498 | 0.045929737 |
| | GAGCTCTACTTACAGAAC | | | |
| | AGCCCTGAGGCCTG | | | |
| 1020 | TTCCCAGGCTGGAACCA | N | 0.025665689 | 0.017387411 |
| | GGGTCTCTCTTTACCTCC | | | |
| | TACCCCATGGTGGCA | | | |
| 1021 | GCTACAGAAACAAGGTC | P | 0.01326295 | 0.01077719 |
| | GTCGGCTGGCGGAGCGG | | | |
| | CTTGGAAAAAGACCTT | | | |
| 1022 | CGAAATTCCCCAGAAATT | N | 0.032498303 | 0.005482439 |
| | AGTAGTAAGTGGGGTCTT | | | |
| | TGTGGGTTGGGAAG | | | |
| 1023 | GCAGCTGGTTTCTTTAAA | N | 0.049311193 | 0.003275167 |
| | GGTGTTGGGAAAGGTTTA | | | |
| | GTAGGAGCGGTAGC | | | |
| 1024 | AGATTGTGGAATGGGTGT | N | 0.045081493 | 0.007970505 |
| | AAGACAATTGGTAGGGG | | | |
| | GTGAAAGTGGGTTTG | | | |
| 1025 | TCGGTCCCGAGACGTTC | P | 0.024879954 | 0.00026266 |
| | CGCCAGCGTTTCCGGCA | | | |
| | GTTCCGCTACCAGGAT | | | |
| 1026 | CTGTGTTTCTGCAAGTGC | N | 0.013613554 | 0.003696673 |
| | CATCCTTGTACAGTGTTA | | | |
| | AGAGGGTAACATGG | | | |
| 1027 | CTCGGCCATACTCACTGC | N | 0.004495385 | 0.002711004 |
| | CCCCCTTGGGCACCCAC | | | |
| | TCACCCTAGAATAAA | | | |
| 1028 | CCGTGGTACATACTGGGT | P | 0.038077447 | 0.015747273 |
| | CAGGCACTAGCATGGAG | | | |
| | GAGGGTCACAGAGTG | | | |
| 1029 | ACTGGGTTTGGCCTGGA | N | 0.003754139 | 0.006775698 |
| | CAGCACTGATTTGTGGAT | | | |
| | GTGGATGGGGGCACG | | | |
| 1030 | GCAGGGGATCGACATTG | N | 0.00428251 | 0.005859953 |
| | AGACCAAGATGCACGTC | | | |
| | CGCTTCCTTAACATGG | | | |
| 1031 | ATGGATCATCTGAGCCTC | N | 0.017390796 | 0.003412525 |
| | AGGAGGTTGAGGCTGCA | | | |
| | GTGAGCTGTGACTGC | | | |
| 1032 | CCAGGAGTTTGAATCCAT | P | 0.019137014 | 0.043400312 |
| | CCTGGGCAGCATACTGA | | | |
| | GACCCTGCCTTTAAA | | | |
| 1033 | CTACCCATGGTAGACTCA | P | 0.028370217 | 0.001500458 |
| | CAGCTGCCTGTGGTGAA | | | |
| | CTTTGGCTCCCTGCC | | | |
| 1034 | GCTTGGGTACACTTCTCT | N | 0.041813078 | 0.017724472 |
| | TAAGTGGTCTAGTCAAGG | | | |
| | AACCTCMGTCATG | | | |
| 1035 | AGCCCTTCAGCTGCCTG | P | 0.02589747 | 0.00970553 |
| | CACAACCCCTGACATTGG | | | |
| | CTGCTGGTGACTCAA | | | |
| 1036 | ACTACACCTTATTTGGTC | P | 0.019617455 | 0.002051209 |
| | GCAGCTACTGAGGGATG | | | |
| | AACGAAAGCCCCCTC | | | |
| 1037 | CCCATGAAGAAAGCCCCT | P | 0.009051906 | 0.004414306 |
| | CGTTGCCCAGCACTGTCT | | | |
| | GCGTCTGCTCTTCT | | | |
| 1038 | CCCCAAGACCTAAGGGTT | N | 0.029101462 | 0.017192608 |
| | TTATCTCCTCCCCTTGAA | | | |
| | TATGGGTGGCTCTG | | | |
| 1039 | CACTTGAGGACCCTGGG | N | 0.025548744 | 0.000413143 |
| | GAGAGATGGGGGCGGG | | | |
| | GAAAATGGAGGTATGAA | | | |
| 1040 | ACCTAGGATGGGGTTTCT | N | 0.034390037 | 0.000189729 |
| | CTAATTGCTAATCACAAC | | | |
| | CCCACTGGGTCATG | | | |
| 1041 | GAGTGCTATCCACCAGG | P | 0.022535845 | 0.015026122 |
| | CATGAAAGTCCAAGTGCG | | | |
| | GTATGGACGAGGGAA | | | |
| 1042 | TCCTTGGGGGCAGGGCC | N | 0.023540873 | 0.001212794 |
| | TTTGTCTGTCTCATCTCT | | | |
| | GTATTCCCAAATGCC | | | |
| 1043 | GACCATCCGAAACCTGC | N | 0.00587747 | 0.00734318 |
| | GTCCCTGGTGATGTTCTC | | | |
| | AAGCCTCGGAAGTGG | | | |
| 1044 | GTGTTCCTGAAGCTGCTG | P | 0.033438847 | 0.010382953 |
| | TGTCCTCTAGTTGAGTTT | | | |
| | CTGGCGCCCCTGCC | | | |
| 1045 | GCTAGTCGCATACCCGG | P | 0.024351103 | 0.004537936 |
| | AGTAACACCTGCCGCCAT | | | |
| | CATCAATCTGCTGAG | | | |
| 1046 | AGCCCTGCTGCAAAGAT | P | 0.027911092 | 0.026951183 |
| | GGTCAACGTACCTAAAAC | | | |
| | CCGAAGAACCTTCTG | | | |
| 1047 | GATCAGAAAAGCAGAAAG | N | 0.039055523 | 0.00266941 |
| | AGAGAGTGGCCGGATGG | | | |
| | GGCTGAGGGGAGAAA | | | |
| 1048 | CTATTTGCAGGATGAGTT | P | 0.049297126 | 0.005131774 |
| | GGGCAGGGAAAAGGGTC | | | |
| | AGGGTTCATCAGGTG | | | |
| 1049 | TCTGTGCTACCTTATTAA | N | 0.018181769 | 0.008090838 |
| | CTCACAGCAGGCTTACTG | | | |
| | AATGGCTTCATTTC | | | |
| 1050 | CACATTCTTTTTTGGTGTT | N | 0.044161672 | 0.005928323 |
| | CATAGCTTCTTCTCATAC | | | |
| | AGGTGCCAGACAC | | | |
| 1051 | GCTCCCTAGAGGCCCAG | N | 0.029680593 | 0.000117339 |
| | CGCAGCCGCAGCGGACA | | | |
| | AAGGAGCATGTCCGCG | | | |
| 1052 | AGTCCAGGTTCAAGACTA | N | 0.034317358 | 0.034047835 |
| | GCCTGGGCAACATGGCA | | | |
| | AGACCCTGTCCCTAT | | | |
| 1053 | CACCTCCACTGTGATGTA | N | 0.045534716 | 0.045835854 |
| | TGTCCGCTCCCTCGTCTG | | | |
| | TTCCCCCAGGATCT | | | |
| 1054 | CATGTTCGGGGCCACGT | N | 0.039472934 | 0.006554487 |
| | TGTTGTATGTATTGATGT | | | |
| | ACAGCCTTGAATGTG | | | |
| 1055 | AGCCTTAGTCCAGGGGT | N | 0.037499714 | 0.00515636 |
| | GTGGCTCTGTCCGGGTG | | | |
| | CAGTATGCAGTCATGT | | | |
| 1056 | GTCCCTGTGGTAGAAAAC | N | 0.022645668 | 0.004143538 |
| | TTACTCTTTATGCCTGGT | | | |
| | GCAGTATAATTCCC | | | |
| 1057 | CCAGTATTACGGTGCCTC | N | 0.038685845 | 0.028206087 |
| | TTCTCTGCCCCCTTTCCC | | | |
| | AGGGTATCTGTGGG | | | |
| 1058 | GATTGTGCCACCGTACTC | N | 0.023020868 | 0.027602551 |
| | CAGCCTGGGCGACAAGA | | | |
| | GTTAGACCCTATCTC | | | |
| 1059 | CAATTTTGGCCTCTGTGA | N | 0.020169682 | 0.00510351 |
| | TCCTCTTCGCCAATCCCA | | | |
| | TCAGCCCCATGCAG | | | |
| 1060 | AGGCCGAAGCCAGCGCC | P | 0.047913941 | 0.018372791 |
| | CAGCTTTCCTCACTGTTC | | | |
| | CTGTGGAGGATGTCT | | | |
| 1061 | GATCTCCAGGCTTGGCCT | N | 0.018301749 | 0.049741543 |
| | CCAGAGCAGCCCACACC | | | |
| | AACCCCAAAATAAAA | | | |
| 1062 | CTACAGGTCCCCTCTGAG | N | 0.009233746 | 0.00424888 |
| | CCCTCTCACCTTGTCCTG | | | |
| | TGGAAGAAGCACAG | | | |
| 1063 | TGTGTCACAGCCAGAGG | N | 0.047297434 | 0.023687073 |
| | GACAAAGTGTGGGTGAT | | | |
| | CCTGGAGACGCCAGTT | | | |
| 1064 | CTGGGGCCTGTGTAGCC | P | 0.031505737 | 0.003700105 |
| | AGTGGGTGCTATTCTGTG | | | |
| | AAACTAATCATAAGC | | | |
| 1065 | ATGGTCTGGGAGCTGTTC | N | 0.037376141 | 0.000835667 |
| | TGGGGCAGGGGGAATAT | | | |
| | GCAAGCTAAAGCCCC | | | |
| 1066 | GAGGTGGGGAGTTCGAG | N | 0.039959638 | 0.00728954 |
| | ACCAGCCTGATCAACATG | | | |
| | GAGAAAACCTGTCTC | | | |
| 1067 | GAAGGCCAGACTTACTCA | N | 0.047431494 | 0.017758564 |
| | TTTTTCTCCCCCAAGTGA | | | |
| | GCTGCAAGAGGCCC | | | |
| 1068 | GGGCCTCTCTGGTATGG | N | 0.024793526 | 0.000222487 |
| | GCAATAGGCAAGCTCCT | | | |
| | GGGGTCTGGTTATGTG | | | |
| 1069 | CTCCCTGCCCCTGGAGG | P | 0.021793134 | 0.024454086 |
| | TTGTCTTCAAGCTGTGGA | | | |
| | CTTCTGGGATTTGCA | | | |
| 1070 | GGCCGAGAACTGGAAAC | N | 0.048520596 | 3.14646E−05 |
| | AGCCTTTCCCTCATTTTC | | | |
| | TGTGTATTGGTGATG | | | |
| 1071 | GATTAGGCCCTGTTCAGC | P | 0.023407336 | 0.002686615 |
| | CATGCAGGGGTGTTGGT | | | |
| | TTATGCGTGCTGCAG | | | |
| 1072 | GCGACTCTAGTGGCCTG | N | 0.043836203 | 0.002869152 |
| | AGGAGATGTATTTATAGG | | | |
| | CCCCCAGCAGGGCTG | | | |
| 1073 | GGCCGCACACTGGTGGT | P | 0.028329248 | 0.000436968 |
| | CCATGAAAAAGCAGATGA | | | |
| | CTTGGGCAAAGGTGG | | | |
| 1074 | CCTCACCAGTGCTTCATA | N | 0.028442468 | 0.009376793 |
| | TACCATGATATAATCTGA | | | |
| | AAGGGGCAGATTAA | | | |
| 1075 | CACTCAAGAGACTTATAG | N | 0.026412581 | 0.03446177 |
| | CCACACAACCAATCTCTG | | | |
| | CTTCAGACTCTGGG | | | |
| 1076 | AAGAGTGAGTCTGAGCA | N | 0.045662901 | 7.13847E−07 |
| | CGAGTTGCAGCCAGGGC | | | |
| | CAGTGGGAGGGGGTTT | | | |
| 1077 | GAGGTAGTCAGTGGCGC | N | 0.044737703 | 2.43603E−05 |
| | ACAAAGGGTAACAAGCA | | | |
| | GTGATAGTGGGGATGC | | | |
| 1078 | GGGCCCTCATGCTGGCT | P | 0.028313626 | 0.023945809 |
| | TTTCACCCCAGAGGACAC | | | |
| | AGGCAGCTTCCAAAA | | | |
| 1079 | AGGCTCAGAGGACCATA | N | 0.024634501 | 0.001973366 |
| | GGAGGTTTTAAGATTTAT | | | |
| | GTTTAGTCCGATAGG | | | |
| 1080 | AGGCCGTGTCAGCACGA | N | 0.026470289 | 0.005900536 |
| | TGGACCCCAAACAGACC | | | |
| | ACCCTCCTGTGTCTTG | | | |
| 1081 | TAGGAGAGCCTGTGCTG | P | 0.032231481 | 0.020076301 |
| | GGTGCCAAGATGGAGAC | | | |
| | AGCGGCTACCTTGCTA | | | |
| 1082 | GCGGAGAGTTCACGAAC | N | 0.045607507 | 0.044178243 |
| | TGTGCCCAACGCATGTTA | | | |
| | TAGCCAGGGTCCTAC | | | |
| 1083 | CTCCTCTTCCGGGTCCGT | P | 0.001482729 | 0.000168775 |
| | GGGCGAGTCTTCATCTAA | | | |
| | GGGACCAAGATACT | | | |
| 1084 | CACTTGGACCTGGGAGG | N | 0.025813389 | 0.010101332 |
| | CAGAGGTTTCAGTGAGC | | | |
| | CCAGATCGCGACACTG | | | |
| 1085 | CAGTTCTGCATCTGATAC | N | 0.020933213 | 0.03245218 |
| | CGTCTCCTTTCCCTGAAG | | | |
| | TCTGGCACACCATG | | | |
| 1086 | AAAGGCTTGCTTGAGGG | P | 0.049588259 | 0.012252992 |
| | AGCTTATAACTTGCACCT | | | |
| | AGAGGGTTTACCCAC | | | |
| 1087 | CTCAGCGTCTCCCCATGC | P | 0.046286293 | 0.021964029 |
| | TGGGCTCACTACATGGG | | | |
| | CCAGCCCTTGCTCTA | | | |
| 1088 | GTGACAGTGAAGACATGA | P | 0.043809066 | 0.043383402 |
| | AGATGAGAGCTAAGCAG | | | |
| | CTCCTGGTTGCCTGG | | | |
| 1089 | CGGCACCATTGAGTACAT | P | 0.022772857 | 0.002906623 |
| | GCGAGACCCTGACGACC | | | |
| | AGTACAAGCTCACGG | | | |
| 1090 | ACTCAAGGGTGGTCAGC | N | 0.025117718 | 0.002101885 |
| | TCAATGCTACACAGAGCA | | | |
| | CGGACTTTTGGATTC | | | |
| 1091 | TGCAGATGAGATCCAGGT | P | 0.046739135 | 0.000475238 |
| | TGGCTTTGGCCGGGTAG | | | |
| | GCAAGCACTTCTGGG | | | |
| 1092 | GCTGCCCTGTGGGTCTT | P | 0.032740791 | 0.009199183 |
| | GCTCAATACTGTTCATAC | | | |
| | CTGGAGAGAGAAGGT | | | |
| 1093 | ACATCCAAAGACGCACAA | P | 0.011204926 | 0.000961497 |
| | GACCAGCCACTGCTACC | | | |
| | GCATCACGTACCGCC | | | |
| 1094 | GAGCGAATGCCACCCCG | P | 0.030449768 | 0.004061836 |
| | AAAGGATAGCATACACGC | | | |
| | TGGTGGGCCAATGAA | | | |
| 1095 | TTGAGAGGCCGAGGCTG | N | 0.043089869 | 0.02241793 |
| | GCAGGTCACTTGAGGCC | | | |
| | AGGAGTTCAAGACCAG | | | |
| 1096 | GGGAAGACTGGAGCCTA | P | 0.043548037 | 0.006514981 |
| | AGCTGCCTGCTACTGGG | | | |
| | CTTTACATGGTGACAG | | | |
| 1097 | CATGGTCACACATAAAGT | N | 0.039705325 | 0.010220149 |
| | TGCAGTTAGGAAAGGGAT | | | |
| | GGGCAGGGAAAAAC | | | |
| 1098 | GCAGAGCATTCAGTGCC | P | 0.015576617 | 0.004235995 |
| | ACGGTTTAGGTGAAGTCG | | | |
| | CTGCATATGTGACTG | | | |
| 1099 | GCAGAAGAGGGTGTGTT | N | 0.048310539 | 0.033571502 |
| | TGAAATCATCGGAGTCAG | | | |
| | CCAGGAGCTGTCACC | | | |
| 1100 | ATCAACAAGGTGGTGTGA | P | 0.037717417 | 0.005415172 |
| | TGGCTCCTGCACAGGCC | | | |
| | CGACATAGGATGAGG | | | |
| 1101 | GTATGTCAAACTGCCAGT | N | 0.019365062 | 0.014179763 |
| | AAGCCAGCCCCTCACCC | | | |
| | TCTGATAGATATTCC | | | |
| 1102 | CAGTCGCAGCTCTTTGGA | P | 0.027055544 | 0.00263818 |
| | GGTGACTCGTGTTCCAG | | | |
| | GTGGATCCCTCTCTG | | | |
| 1103 | AGCAGCTTGTCTGGCGT | P | 0.006306624 | 0.000845437 |
| | CAACTGGCTTTCAGAGTG | | | |
| | CTGACCCCTCATCAC | | | |
| 1104 | TGGAGGAAGTTCTACACT | N | 0.034466407 | 0.010019144 |
| | CGCCTCACCAACAGCCG | | | |
| | ACACGGTGAGACCAC | | | |
| 1105 | CTGTGAGTGTCTCTAGGG | N | 0.046000559 | 0.002303766 |
| | TGATACGTGGGTGAGAAA | | | |
| | GGTCCTGGTCCGCG | | | |
| 1106 | CCTCAGTCGGTGAAGAA | P | 0.025131185 | 0.024227633 |
| | CAAGGTTCTGGAGGTGA | | | |
| | AGTACCAGAGGCTGCG | | | |
| 1107 | GGCCATGCCTCTGCTCC | P | 0.01350372 | 0.003204862 |
| | ATACTTTGGAAAGGAAGA | | | |
| | ACCCTCAGTGGCTCC | | | |
| 1108 | GCTTCAGCTTTCGGACTC | P | 0.027575924 | 0.008663292 |
| | TGGTTCTTGGATCGTGTC | | | |
| | CTCTCCCCCTCGCC | | | |
| 1109 | ACCGCTTAAACCTGGGA | N | 0.045532763 | 0.019081339 |
| | GGTGGAGGTTGCAGTGA | | | |
| | CCCGAGATCGAGATCA | | | |
| 1110 | GAGGGACATGCTTCCCC | P | 0.005399088 | 0.000599491 |
| | TTGTCCACCTTTGCAGCC | | | |
| | TGTTTCTGTCATGTA | | | |
| 1111 | CTCCAGCCCACGTTCTCT | N | 0.004696751 | 0.005733364 |
| | CTGCCTGTGAGCCAGTCT | | | |
| | AGTTCCTGATGACC | | | |
| 1112 | TACACTAGGGGGTCCTAC | N | 0.047550849 | 0.000472868 |
| | AGCTACGTGGCCGTGGG | | | |
| | CAGTACTGGGGGCGA | | | |
| 1113 | GGACAGCATGTCCCTGC | P | 0.03097545 | 0.00643237 |
| | TCTTCCGCCTGCTCACCA | | | |
| | AGCTCTGGATCTGCT | | | |
| 1114 | CGACACTGTGGACTGAA | N | 0.039877769 | 0.004742872 |
| | CACACTGAAGCTCTGATG | | | |
| | GGAAAACCTGGTGAC | | | |
| 1115 | GAGTGGCACTGATAACTG | N | 0.012106144 | 0.00707376 |
| | GTGAAGCCTACAGCCATC | | | |
| | CGCCCAAAAGTCTG | | | |
| 1116 | CGAATGTGATTGGAACAT | N | 0.014422232 | 0.00304009 |
| | TTGGGGAGCACCCAGAG | | | |
| | GGATTTCTCAGTGGG | | | |
| 1117 | GCAACCATGCAAAAGAAA | N | 0.031803103 | 0.002697089 |
| | TTAATTTGGCCAGGCACA | | | |
| | GTGGCTCATGCCTG | | | |
| 1118 | AGTGGCACTCGACGAAA | P | 0.019666667 | 0.01243194 |
| | AGGAGACTATGCCAGTTA | | | |
| | CTACCAGGGCCTATG | | | |
| 1119 | GGATCTTTGTCTTCTGGC | N | 0.041751715 | 0.01183193 |
| | TGGAGGTGCTTTTGGAG | | | |
| | GTTGGGTGCTGGGCA | | | |
| 1120 | GCTCTGCGTGTGGTCCG | N | 0.022619007 | 0.026271866 |
| | TATGGAAAGCCTGGTAGC | | | |
| | CCTGCGAGTTAAGTA | | | |
| 1121 | TGCTGAAGTTTTTGGCCA | P | 0.014993772 | 0.008550214 |
| | GCTTTAGTTTGAGGACTC | | | |
| | CTTGATAAGCTTGC | | | |
| 1122 | TGCAGTGAACTCCAGAGA | N | 0.024405392 | 0.00555678 |
| | CCTAGGGGATGTGGCTG | | | |
| | TGTCGGCAGCAAGAG | | | |
| 1123 | AAGGAAAAGGTCAGTGC | N | 0.015265932 | 0.004918118 |
| | AGTCAGGAGAGACAGGT | | | |
| | CCCGAAAGACGCCCTC | | | |
| 1124 | TCGGGGAAACTGTGTGT | N | 0.026870945 | 0.027170535 |
| | GCTGAAGAGTACGTGGG | | | |
| | AGCTCTCTGTGCTATC | | | |
| 1125 | AAGAGGCTGCGGTGAGC | N | 0.046323914 | 0.0144836 |
| | CAATTTAGAGCCCAAAGA | | | |
| | GCCCCGAGGGAACCT | | | |
| 1126 | TGTGCCCGATGCGTGCC | N | 0.034773849 | 0.027149099 |
| | CAAGGACAACGCCATTAA | | | |
| | GAAATTCGTCATTCG | | | |
| 1127 | AGCAGTTCCTGACGGAG | P | 0.045122451 | 0.024123594 |
| | CTGACCAGACTTTTCCAG | | | |
| | AAGTGCCGGACGTCG | | | |
| 1128 | GGTGATGTATGGCTAAGA | N | 0.009866158 | 0.001858793 |
| | TTTCACTTTAAGCAGTCG | | | |
| | TGAACTGTGCGAGC | | | |
| 1129 | TCAGCATGAGGTGGAGC | N | 0.01409167 | 0.048823964 |
| | AGTGACCAGGTGGAGCA | | | |
| | GTGACCAGGACGCCTC | | | |
| 1130 | CCATCTGCTTAACCCTTG | P | 0.015169214 | 0.002204865 |
| | GCTCCACCATAAGGCACT | | | |
| | GGGACTCGGATTTC | | | |
| 1131 | AACAGTCTCTCCGCCCC | P | 0.006295555 | 0.017049578 |
| | GCACCAGATCAAGTAGTT | | | |
| | TGGACATCACCCTAC | | | |
| 1132 | CCTCTGTTGCAGTCTTTT | N | 0.041125294 | 3.35379E−05 |
| | TAAGGGGTGGGCCAATC | | | |
| | ATAATGAAGAGGGGC | | | |
| 1133 | TTCCCAGGGCAAGGCTG | P | 0.021135453 | 0.019288379 |
| | ATCTGTTGCCGTATTAGT | | | |
| | CCGTTTTCACACAGC | | | |
| 1134 | CTACCCTCATTAACAATT | N | 0.023930555 | 0.011891939 |
| | AGCAGGGCACTGGCCAG | | | |
| | AGTTTGTACCCTGTG | | | |
| 1135 | GCGCAACACCTAGGAGC | N | 0.006433886 | 0.003532166 |
| | CCAAAAATAAGCAGCACG | | | |
| | ACGGAACTTTCAGCC | | | |
| 1136 | TCTAGTGGATTGGTTTTC | N | 0.008539893 | 0.002553882 |
| | AACATCGTGCCTGCCGAT | | | |
| | ATGCCTACAGAATC | | | |
| 1137 | AGCCTGCTCGGCTGTAT | P | 0.041600108 | 0.012386311 |
| | GTTTCTCGCACGCTCACC | | | |
| | CGCTTCAGTGCGGCT | | | |
| 1138 | CATCACTGGGAAGCAGG | N | 0.036852633 | 1.13163E−05 |
| | CAGTGTCTTGGGTGGGG | | | |
| | GCTTGGTCAGTATCCT | | | |
| 1139 | CGCCCATTGAGCAGGAG | N | 0.035787476 | 0.004099986 |
| | CCATCAAAGAAGCAGAAG | | | |
| | AAGCAACATGAGGGC | | | |
| 1140 | GACCTGGACGCAGAGGC | P | 0.044975177 | 0.001936395 |
| | CCGTCATCTGGCGGATG | | | |
| | AGGCTAAGAATCTTGT | | | |
| 1141 | GGAGCTGCCAGGGATGC | P | 0.02867681 | 0.005464334 |
| | TTTACAGGGATTTAAGAT | | | |
| | CACACCGTCCCATGC | | | |
| 1142 | CCCCGCTTGTGTCTGAG | N | 0.01146418 | 0.002141017 |
| | GTCGTGTATGTCAAAAAT | | | |
| | AAAGCCGCTAGAAAC | | | |
| 1143 | GCACTTTGGGAGGCTGA | N | 0.034930505 | 0.012013477 |
| | GGCAGACAGATCACGAA | | | |
| | GTCAGGAGATCGAGAC | | | |
| 1144 | GCTGGGAGCAGTTGCAG | P | 0.048953115 | 0.022353363 |
| | GCCACAGTGAAGTGTGTT | | | |
| | GCTTCTGTCACTTTA | | | |
| 1145 | GGACTTCCAACCTTGACT | P | 0.036163817 | 0.011749739 |
| | GCTGAGCTCCTGGCTTA | | | |
| | GCTTCTTGGGTTCCT | | | |
| 1146 | CAGCACTTGTGAAGGATT | N | 0.046604194 | 0.000938006 |
| | GAATGCAGGTTCCAGGT | | | |
| | GGAGGGAAGACGTGG | | | |
| 1147 | GGCCCTGGGGGACCCTC | N | 0.02735808 | 0.029529244 |
| | TGAAGCATTTCTGCCTCA | | | |
| | CTTTATGTCATCTGC | | | |
| 1148 | TAACTTCCAGGAGTTCCT | N | 0.002639909 | 0.015542989 |
| | CATTCTGGTGATAAAGAT | | | |
| | GGGCGTGGCAGCCC | | | |
| 1149 | CTCTTAGGGTGTTGTAGT | N | 0.037777493 | 0.005047962 |
| | AGCTGAAACATGGAGATG | | | |
| | CGTAGCTGTCATGC | | | |
| 1150 | CTCTGCCTGGATGCTGTT | N | 0.001769912 | 0.000740642 |
| | TGTGGTTACCTTGTTGAG | | | |
| | CTCCAGTCTATGAG | | | |
| 1151 | GCAGGATTCTGCAAAATG | P | 0.028372725 | 0.011371306 |
| | TGTCTCACCCACTACTGA | | | |
| | GATTGTTCAGCCCC | | | |
| 1152 | CGCCCAGGGCGGCGTTT | P | 0.014131092 | 0.013805369 |
| | TGCCTAACATCCAGGCC | | | |
| | GTTCTGTTACCAAAGA | | | |
| 1153 | CCGAGCCTCTTGAAGCC | N | 0.027464042 | 0.02086608 |
| | ATTCTTACAGATGATGAA | | | |
| | CCAGACCACGGCCCG | | | |
| 1154 | CCCAACCCTGCTGTTAGG | P | 0.001787264 | 0.000725567 |
| | CCTGCTGTTCCCTTTGCT | | | |
| | CTTGATTAGGAGAG | | | |
| 1155 | TCCGGAGCCGCCCTCCA | P | 0.039149351 | 0.014771689 |
| | TTGTGGGTTCCTGAGAGT | | | |
| | AGGACACATTGCCAT | | | |
| 1156 | CAGGGAGCAGTCTTCCAT | N | 0.032287036 | 0.006937537 |
| | CATGCTGAATTTTGTCTT | | | |
| | CCAGGAGCTGCCCC | | | |
| 1157 | GGGATGGACCTGGAAAC | P | 0.023853484 | 0.011460922 |
| | AAGCACCTCCCCAAACAC | | | |
| | ATCACCACTCCCTAG | | | |
| 1158 | TGAGTGAGGAGATGCTT | N | 0.006336264 | 0.033454534 |
| | GCCGTGATGACGCTGGG | | | |
| | CACAGAGGGTCAGGTC | | | |
| 1159 | CTGCCCAGTGAGATGGA | N | 0.025729022 | 0.016988936 |
| | GGACGCTAGAGAAAGTG | | | |
| | CTGAGTGTCCCGAGAG | | | |
| 1160 | GTAACTCCCCCCAGGTAC | P | 0.011518269 | 0.001629918 |
| | GATAGGGACTGAATATGG | | | |
| | ACCCTGCTGAAAGC | | | |
| 1161 | GCTCTAAGTAATGTGATT | N | 0.025187047 | 1.92381E−05 |
| | CTTCTAAAGCAAAGTCAT | | | |
| | TGGATGGGAGGAGG | | | |
| 1162 | CTGGAGTTTGAGACCAG | N | 0.028802543 | 0.029385666 |
| | CCTGAGCAACATGGTGA | | | |
| | GACCCCATCTCTACAA | | | |
| 1163 | CAGTGGAGTGTTCTGCAA | P | 0.029151815 | 0.003673068 |
| | AGCCCGATTCTCTGCAGC | | | |
| | TTTAAGACTGGACA | | | |
| 1164 | GTTGCATTTCATGGGCCT | N | 0.029561039 | 0.000297704 |
| | GGGGGTTTCCTAGCAGA | | | |
| | GGATATTGGAGCCCC | | | |
| 1165 | GCTCCCACCCCAACGAA | N | 0.027068674 | 0.002725252 |
| | GGCTCATAGGAAAACAGT | | | |
| | GCATGTGCTTGGTGG | | | |
| 1166 | CTGGCTCTGAAGACCACA | P | 0.033503443 | 0.036059277 |
| | GATGCAAGCAATGAGGA | | | |
| | ATACAGCCTGTGGGT | | | |
| 1167 | TTGCTGTGCGGAGGGCC | N | 0.020947489 | 0.012152277 |
| | TGCTATGGTGTGCTGCG | | | |
| | GTTCATCATGGAGAGT | | | |
| 1168 | TCTGAGCCTCCGTCGCC | P | 0.010030101 | 0.004147422 |
| | CCTCCTGTTGGGTAAGG | | | |
| | GTGTTGAGTGTGACTT | | | |
| 1169 | TCTCAGCCCCCAGGCTG | N | 0.03709215 | 0.000968344 |
| | TGAGCTCCTTGGGGGCA | | | |
| | GGCCCTCAATAAATGT | | | |
| 1170 | GCGGAGCTCGGGATTGG | P | 0.031497049 | 0.016714108 |
| | CTAAACTCCCATAGTATT | | | |
| | TATGGTGGCCGCCGG | | | |
| 1171 | GGTGATAGTCAGAGAGT | N | 0.033152343 | 0.000146544 |
| | GGTGTTTTTGTTCAGGTG | | | |
| | GGAAGGATTGGAAAC | | | |
| 1172 | TCTGGGGCTGTGGTCAC | P | 0.02529593 | 0.017219564 |
| | CCTCGAATGCGTGGAGA | | | |
| | AGCTGATTCGGAAGGA | | | |
| 1173 | CTAACCAAACCCCAGACA | N | 0.022507591 | 0.002843893 |
| | TAGGGAGTCATTTGGAGA | | | |
| | AAGCCTGTATGTGG | | | |
| 1174 | CAGTCTAGCTGATTGTTC | N | 0.02765978 | 0.018657201 |
| | CACCAAAGAATCCAGCCT | | | |
| | GCTGCTTATTCGAG | | | |
| 1175 | AGTGTGTGTCCTCTGAGG | N | 0.015335541 | 0.041677816 |
| | TGCTTGAGAAAGTGTACA | | | |
| | CTGCAGAACTGCCC | | | |
| 1176 | TTCTGGTTGCCAGGAGAC | N | 0.026451997 | 0.028367603 |
| | AGCAAGCAAAGCCAGCA | | | |
| | GGACATGAAGTTGCT | | | |
| 1177 | CCCCTTCCTAGCTGTATG | N | 0.025493746 | 0.025166976 |
| | ACCTTGATTGTGTGCCTT | | | |
| | AACCGCTCTGTGAC | | | |
| 1178 | GATTCCCCGCTGCGTCTA | N | 0.047061367 | 0.014429807 |
| | AAATCAAAGCGCCAATCT | | | |
| | CATAAGTACCAGGC | | | |
| 1179 | GAGGAAGTCCCAAAAAG | N | 0.007547103 | 0.01085825 |
| | GTGCTGTCACTTTAAGTT | | | |
| | CTGGACTTGGGGTTC | | | |
| 1180 | AATGAGGACAGAGAACCT | N | 0.036887891 | 0.019287674 |
| | CAGGTGTTCTTATGCTAG | | | |
| | TGCTTGCTGAGTGC | | | |
| 1181 | CTGTTCCAGTGTCAACTG | N | 0.032742601 | 0.012789267 |
| | CCATGTGCTCTGCTTCAA | | | |
| | GGGGGAACTAGCCT | | | |
| 1182 | GCAGAGGCAGTCTATTG | P | 0.028013548 | 0.004046822 |
| | CAAGGACCTTCTTTGCTG | | | |
| | CCAGTTATCATAGGC | | | |
| 1183 | TCCCACCTGGCATTGCAT | P | 0.025873241 | 0.005245702 |
| | CGTGGGGAGGAACTTCG | | | |
| | GTAGTTATGTGACAC | | | |
| 1184 | GCCAAATCTGAGCATCAG | P | 0.01750223 | 0.005931921 |
| | AAGTCTTTCCAGTCTACC | | | |
| | TGATGCATGATCTC | | | |
| 1185 | CCCTGGGGATAGCTGGG | N | 0.012578813 | 0.000905615 |
| | GCATTTGTCTAGCTGGGC | | | |
| | TACCTTCTAACACTT | | | |
| 1186 | TCTCTTTTCTGCCTCTTAC | N | 0.025537822 | 0.018142309 |
| | ATGTGAATGTTGAGCCCA | | | |
| | CAATCAACAGTGG | | | |
| 1187 | GCTCTCTCTGCCAGAATT | P | 0.008064735 | 0.000749642 |
| | GTGTGCACTCTGTAACAT | | | |
| | CTTTGTGGTAGTCC | | | |
| 1188 | CTGCCAGCTGTTTTACCA | N | 0.041584156 | 0.002256447 |
| | GGGATCCAGAGACATAG | | | |
| | AGGAAGTAGGGGGTG | | | |
| 1189 | GTGCCTGGTCCAATGTG | P | 0.026957527 | 0.001978261 |
| | CTGAGAGGCATGGGCGG | | | |
| | TGCTTTTGTATTGGTG | | | |
| 1190 | CAACTGCTGCGTCCGTC | P | 0.03344237 | 0.02780037 |
| | CTGCCCCGTCTTCTGTGT | | | |
| | TAGATTGAAAGGGAG | | | |
| 1191 | CCAGCCCTGGGAGAACT | P | 0.046611568 | 0.047325681 |
| | GGGTAGCAGGTGGCTGA | | | |
| | CTTCTTTAAGCACCTT | | | |
| 1192 | CCAGGTTTTCCTGGTCTC | P | 0.040747666 | 0.014133667 |
| | AGACCTATGATGACTTGT | | | |
| | CCCTTTGATGTCAC | | | |
| 1193 | AGCCTTTTCTCAAAGCCC | N | 0.048828958 | 0.028695649 |
| | TTTCAGTTACAACCACCC | | | |
| | CACTATGGAATCAG | | | |
| 1194 | AGTTCTGGATGGATTTGG | N | 0.02257594 | 0.005858182 |
| | TGGCCTGACATGATACCC | | | |
| | TGCCAGCTGTGAGG | | | |
| 1195 | CCCTTCCTACTCCCAGAC | N | 0.020856349 | 0.044268054 |
| | ACCCACCCTCGCTTCAGC | | | |
| | CACAGTTTCCTCAT | | | |
| 1196 | GACCAGATCATGCTCCAT | N | 0.011092874 | 0.005313112 |
| | CCAGCCCCACCCAATGG | | | |
| | CCTTTTGTGCTTGTT | | | |
| 1197 | TGCCCACTCGGCCCGGA | P | 0.046970996 | 0.010641627 |
| | ATGACTCGACCAGACAGA | | | |
| | TAAGGATAGAGGGGA | | | |
| 1198 | CATTGGACGGCAGCAAC | N | 0.011880355 | 0.013027233 |
| | TGGGGGCAAGAACAACG | | | |
| | GGAAGATCACCTGTGC | | | |
| 1199 | TCCTTCGACCTCCACTGC | P | 0.036007318 | 0.00928651 |
| | GCCCCACCTCCCTGCCT | | | |
| | GTGTGTGTTATTTCA | | | |
| 1200 | TCTATGAAGCCCACTCAC | P | 0.039220283 | 0.014917512 |
| | TTGCCATTCCAGGGCCAA | | | |
| | AGGACCGGAGGTTT | | | |
| 1201 | CCATACGGGTTGGTTTCA | N | 0.021732006 | 0.008344053 |
| | CCTCCTCAGTCCCTTGCC | | | |
| | TACCCCAGTGAGAG | | | |
| 1202 | CAGAGGCCACGACATAA | P | 0.015830619 | 0.00291066 |
| | AAATTCAGTCCCTTTGTC | | | |
| | CTTCCCCGTGCCTCC | | | |
| 1203 | TGGGGGTAGTTGGAAGG | N | 0.033447401 | 0.000982633 |
| | GACTGAAATTGTGGGGG | | | |
| | GAAGGTAGGAGGCACA | | | |
| 1204 | ATTACATGGGTTTCCCAG | P | 0.048593504 | 0.000654233 |
| | CTGTTTGCGCGGCCTTG | | | |
| | GAGCACCCACAGAGG | | | |
| 1205 | AAACCCGTCAATGTACTA | P | 0.013130837 | 0.003189037 |
| | GGATACTGCTGCGTCATT | | | |
| | ACAGGGCACAGGCC | | | |
| 1206 | TGTGTGTTTTCTGCGGCC | P | 0.020322652 | 0.003959982 |
| | CTGGATAATGCTGTAGCA | | | |
| | TTCAGGGTCGATTG | | | |
| 1207 | ACCTGAGCGCCCCCGTT | N | 0.037945224 | 0.022206464 |
| | ACCTTGAACTTGAGGGAC | | | |
| | CTGTTCTCCACCTTC | | | |
| 1208 | GCCTGTTCTCTGCCATTC | P | 0.010516259 | 0.003830891 |
| | CCTAGTCATCCTGTGCCT | | | |
| | CACCACAGCTTGCT | | | |
| 1209 | ACTTGAGCTGAAAACCCA | P | 0.022069663 | 0.005412735 |
| | GATGGTGTTAACTGGCC | | | |
| | GCCCCACTTTCCGGC | | | |
| 1210 | ATGGGCCCTACAGTCCCT | N | 0.013090539 | 0.00477988 |
| | GGGCTACACCTGCTACA | | | |
| | GCCGGGGTGTCATCA | | | |
| 1211 | GTGTCCAGCCCACCGCC | P | 0.014188321 | 0.007451265 |
| | TGCCGACTTGTGTCATGC | | | |
| | CCTACGTTGGTATAA | | | |
| 1212 | GACTGTCATGTAAGAGGT | N | 0.020822363 | 0.034381021 |
| | GCTCTCCTGGCACCCAG | | | |
| | AGAAAAGGAGCATCC | | | |
| 1213 | CAGCAAGGGTTCCAAATT | N | 0.024933029 | 0.004316736 |
| | AAGAGGAAGCAAAACGC | | | |
| | TGCCCAGATCCTGAG | | | |
| 1214 | CCCACCCCTCCTCCAGG | N | 0.028036265 | 0.018455146 |
| | GCAACCCCTTGGTCCTAC | | | |
| | AGCAAGAAGCCAGAA | | | |
| 1215 | CCCGTCAGGACGTTGAG | N | 0.03956246 | 0.020514495 |
| | GACTTTTCGACCAATTCA | | | |
| | ACCCTTTGCCCCACC | | | |
| 1216 | GTGAACGCGTGCCTAGC | P | 0.02721104 | 0.006264687 |
| | AGAAGAGCTCCCGCACA | | | |
| | TCCATGCCTTTGAACA | | | |
| 1217 | GGTTCTGACAACAGTACC | N | 0.037504888 | 0.046613892 |
| | CATCCCCCACAGTACCCC | | | |
| | TTCAGCTCAGTTTC | | | |
| 1218 | GCCATAAGCGTCTGTGC | N | 0.005596769 | 0.009112082 |
| | GTGGAGTCCCCAATAAAC | | | |
| | CTGTGGTCCTGCCTG | | | |
| 1219 | CCCTGGCCCACCCCCAC | N | 0.008387788 | 0.003693751 |
| | TTTCCAGGGCAAAAAGG | | | |
| | GCCCAGGGTTATAATA | | | |
| 1220 | ATCCCCGTGTATGGCCC | N | 0.026117037 | 0.004148005 |
| | CCCTGCACCTCCTTGTCT | | | |
| | CATCCCCGAAGATCC | | | |
| 1221 | TTAGGAGGGAGGATGTA | N | 0.004261005 | 0.013495366 |
| | AATAGCCGCACAAAGGG | | | |
| | GTCCAACAGCTCTTTG | | | |
| 1222 | TGGGCTGGATATCTTTGT | P | 0.01406002 | 0.00273231 |
| | AGATGTGGACCACCAAG | | | |
| | GGGTTGTTGAAAACT | | | |
| 1223 | ACAAAATACCAAGTCTCC | N | 0.028404503 | 0.044473069 |
| | CCTCTTCATGGGAAAAGT | | | |
| | GGTGAATCCCACCC | | | |
| 1224 | GCATCCTCCTGTGTATGG | P | 0.011820661 | 0.02519089 |
| | AAGAGACAGGTGACCGC | | | |
| | TCCAGGTTGGGTGCT | | | |
| 1225 | CATGTCTTGCCTCAGACC | P | 0.048495659 | 0.007669068 |
| | CTCACACACTGTCCTCTC | | | |
| | TGCCTGCAACACTC | | | |
| 1226 | TTTTTTGAAGATCAGCTC | P | 0.000525348 | 0.002117839 |
| | CGTGGGGCTGGTTTTGG | | | |
| | TCCACAGCATAACAG | | | |
| 1227 | GCATGGGAAGTATGAGA | N | 0.044450639 | 0.002604668 |
| | AGGTGCTTGTGGAAGGG | | | |
| | GGCCCGGCTCCCTAGG | | | |
| 1228 | AGCTGCCATACTTCGACT | P | 0.042172196 | 0.033118877 |
| | TATCAGGATTCTGGCTGG | | | |
| | TGGCCTGCGCGAGG | | | |
| 1229 | GGCCAGGTGCAGACCTT | N | 0.044322403 | 0.003547524 |
| | GGGGGAAGACTTTAAAC | | | |
| | CACCTAGTTCTCCCCG | | | |
| 1230 | TGTGGAATGCACAAAATT | N | 0.014724414 | 0.00068294 |
| | GTGTAGGTGCTGAATGCT | | | |
| | GTAAGGAGTTTAGG | | | |
| 1231 | ACAAGTGAAGGCCATCAA | N | 0.042744632 | 0.01125372 |
| | AGAATTGGGTGACCACGT | | | |
| | GACCAACATGCACG | | | |
| 1232 | AAGTCTCAGGTGGCTGC | N | 0.03983545 | 0.023115908 |
| | GTGTGGTGGCTCATGCC | | | |
| | TGTAATCCCAACATTC | | | |
| 1233 | GAACTTGCCAGATGCAAA | N | 0.020008078 | 0.003789389 |
| | TACCACAGACTCCAAGAA | | | |
| | AACCCGAGTTGGGG | | | |
| 1234 | GAAGGGCCTCCTGCAAA | P | 0.031882036 | 0.008347754 |
| | ACATCCTTCCTTGAAGCC | | | |
| | TAGCACTGAAGTGGA | | | |
| 1235 | CACTTTCCTGCTCCTCTC | N | 0.045671779 | 0.017334654 |
| | TGTCTCTAGCACACAACT | | | |
| | GTGAATGTCCTGTG | | | |
| 1236 | CAAGTCAGTGACCAGAAA | P | 0.031498757 | 0.005550791 |
| | AATCCCACCCCTTGCCCT | | | |
| | TTCCCCAAAGGACC | | | |
| 1237 | AGTTGCTCCTGCCCCCTC | P | 0.028283634 | 0.010012459 |
| | CCTGAACTATTTTGTGCT | | | |
| | GTGTATATCACTGC | | | |
| 1238 | CAGTTGGTCATCAACTAT | N | 0.037606876 | 0.00712266 |
| | TTTCCCTTGACTGCTGTC | | | |
| | CTGGGATGGCCTGC | | | |
| 1239 | GAGTCCCTAAAGGCAGC | P | 0.026323651 | 0.038482504 |
| | AGCTCAAGGATGGCACT | | | |
| | CAGATCTCCATGGCCC | | | |
| 1240 | CCAAGGATCAGTTTGTGT | P | 0.038879608 | 0.012887723 |
| | GTGGAGAAACAGTCCCA | | | |
| | GCCCCTTCAGCCAAC | | | |
| 1241 | GGGAGGTTTGTGAGAGC | P | 0.010248999 | 0.003569823 |
| | GAGGCTGAGCCTACAGA | | | |
| | TGAACTCTTTCTGGCC | | | |
| 1242 | CCACACACTCACCACTCC | N | 0.006116041 | 0.004639072 |
| | CAGCTTCTCGTGTCCAGT | | | |
| | GAAACCCCTGAACC | | | |
| 1243 | TCCCTGGAGTACGGGAA | P | 0.022584073 | 0.006097973 |
| | GGCTGAGCTGGAGATTC | | | |
| | AGAAAGACGCCCTGGA | | | |
| 1244 | GGGTACTTTCAGTACACA | N | 0.042179308 | 0.04293739 |
| | ACACCCCTAAGATTTCCC | | | |
| | AGTGGTCCGAGCAG | | | |
| 1245 | GCCCCAGGGCGTGGCCG | N | 0.034947874 | 0.023260146 |
| | CTGTTACAGAAACAATAA | | | |
| | ACCCTGATGGGCATG | | | |
| 1246 | CACAGAAATGCTTGCAGC | P | 0.019185719 | 0.012471169 |
| | CTAAGGCAGGGTTTTCAG | | | |
| | ACCGTGGGTCCCAG | | | |
| 1247 | CATGGATTGCGGATATTT | P | 0.003660639 | 0.005766003 |
| | ACCTCTGTCCCCAAACGC | | | |
| | TGACCACGCCCTGG | | | |
| 1248 | GGGGCAGTTTAAAGCAC | N | 0.032514469 | 0.009666582 |
| | AATGTCTCACATGGGACA | | | |
| | AAGTTCCAAAATGCC | | | |
| 1249 | CCCAAAGGCCACATCCAA | N | 0.023081538 | 0.007847408 |
| | GACAGGCAATAATGAGCA | | | |
| | GAGTTTACAGCTCC | | | |
| 1250 | CGGTGGTTGATGGCGCC | P | 0.006399845 | 0.005043293 |
| | TTCAAAGAGGTGAAGCTG | | | |
| | TCGGACTACAAAGGG | | | |
| 1251 | CAAGGGTGCATGGTCCA | N | 0.029858222 | 0.033385679 |
| | ATGTTCTCAGAGGCATGG | | | |
| | GTGGTGCTTTTGTGC | | | |
| 1252 | CAGCTCCGTCCCCAGCG | P | 0.0040143 | 0.001752433 |
| | CTCATGGTGTTGAAACTG | | | |
| | TCTGTCATGCACCAC | | | |
| 1253 | CTGGAGTAAGCTACAGG | N | 0.030690305 | 0.003254254 |
| | ATCTAAAGCAGCCCTTTT | | | |
| | TACAGTCTAGTTAGG | | | |
| 1254 | TCTTCAGGTTCTCCTCTG | P | 0.036563967 | 0.023118757 |
| | TCATTCGGTCAGCCGTCT | | | |
| | CAGTCCATTTGCGG | | | |
| 1255 | TCTTCGTGGCCTCAATGC | N | 0.009754171 | 0.023173652 |
| | CCTCCTTTATCCTCATCTT | | | |
| | TCTTCTATGCAGA | | | |
| 1256 | GGGCTATTAGAATGGGA | N | 0.004617656 | 0.001323707 |
| | GTGCAAAACTGATGCAGT | | | |
| | AGAAGCCCTTACGGC | | | |
| 1257 | CCCATAAAACAGGGTGTG | N | 0.008301395 | 0.009015016 |
| | AAAGGCATCTCAGCGGC | | | |
| | TGCCCCACCATGGCT | | | |
| 1258 | AGGCTCCCTTCTGAGCCT | P | 0.008372085 | 0.013817965 |
| | CTCCTGCTGCTGACCTGA | | | |
| | TCACCTCTGGCTTT | | | |
| 1259 | AAAGCCGGTGCAGGCTC | P | 0.024321007 | 0.000447009 |
| | TGCTGTCATCGTCAGGAC | | | |
| | TCCTTGTGGGCTGTG | | | |
| 1260 | CCGGCGGAAATGAATGC | N | 0.010749145 | 0.003876485 |
| | TGATACCCTAGTAGTCCC | | | |
| | CAGCTCCCAAACACT | | | |
| 1261 | TCTGCACACCCTTGCTCC | N | 0.017604888 | 0.040367999 |
| | CCAAGTACCCGCCCACC | | | |
| | GTTTCCATGGTCGAA | | | |
| 1262 | CGGGAGTGCCGGGCAG | N | 0.0477823 | 0.041978252 |
| | GAGCATGGGGTGCTTGG | | | |
| | TTGTTTCCTTCCTAATA | | | |
| 1263 | GCGTGAAAAACTGTTGAT | N | 0.048101589 | 0.017064373 |
| | AGCAGCAAGAGAAGGGC | | | |
| | AGCAGTCGTTCCATC | | | |
| 1264 | TCCAGTCTGAAGGGAATA | P | 0.0144343 | 0.026196114 |
| | GCTCGCCAGCAGGTTTT | | | |
| | GATGCCAGTGTGAGC | | | |
| 1265 | ACAGCGCCATCTCTCCCT | N | 0.031976496 | 0.010950478 |
| | GAGAATAAAGCCGATAGC | | | |
| | CACCTCCTCCGGCT | | | |
| 1266 | AAGTTCCTGGCTTCTGTG | N | 0.002675617 | 0.001467131 |
| | AGCACCGTGCTGACCTC | | | |
| | CAAATACCGTTAAGC | | | |
| 1267 | GACCTAAGGTCATTTGCT | N | 0.031778044 | 0.012279666 |
| | TTCAATTAGAGGCTCCAG | | | |
| | AGTCTTCATAGTGG | | | |
| 1268 | CTCTATTCTACCAGTGCC | P | 0.024853108 | 0.040729757 |
| | TCCCCGCTTGTGTTGCTC | | | |
| | AGGACGTGGGGCTT | | | |
| 1269 | CATGGGTCTCCAGGAAG | N | 0.036805994 | 0.000608441 |
| | TGACCTTGGATGGGGGT | | | |
| | GGGAAGGGGCTGCTGG | | | |
| 1270 | CCCCAAACTAAGCCATTT | N | 0.046649294 | 0.024389688 |
| | GAAACAAGATTCTCTCCA | | | |
| | TTGCAGTTTGTAGC | | | |
| 1271 | GATCTTCCAGGCCTATGG | N | 0.013249695 | 0.042070273 |
| | ATAGATCAGGAGGCATCA | | | |
| | CTGAGGCCAGGAGC | | | |
| 1272 | CATCCCGTTCTTCGGTTT | P | 0.019030088 | 0.005032268 |
| | GCCATGAGACGATGTGG | | | |
| | GGTTTCCACTGTGTG | | | |
| 1273 | TGCTCGTGGTTTCAGTGT | P | 0.033573178 | 0.035629373 |
| | CCGTGTGTCCATGTGTCT | | | |
| | GCCCTTCAGGAGCT | | | |
| 1274 | GGTCTCAGCCAGCCCTA | P | 0.01979667 | 0.002153455 |
| | GAGACTGCTTCTTGTGTT | | | |
| | TGTGTCATTCTGTCC | | | |
| 1275 | GCGGCACAGTCCCACTT | P | 0.036556034 | 0.038562453 |
| | CCCCATCTCCCCAAGTAG | | | |
| | GTGGTGTTAGAAAAC | | | |
| 1276 | TGGAGGCCGTGGCTATG | P | 0.044651051 | 0.012583354 |
| | GTTGGAGGTCAGCTTCA | | | |
| | GGCCTTCTGGAAGCAT | | | |
| 1277 | CCAGCTCAGTCAAGCCG | N | 0.010139132 | 0.021350342 |
| | CCACATGCCCACAACCTC | | | |
| | ACCAGAGGGAGAATT | | | |
| 1278 | GAGGGTCATCTCTTCTAT | P | 0.024712232 | 0.009604509 |
| | TGTACCTGCAGCACGCTT | | | |
| | CCTGGGCTGTTACA | | | |
| 1279 | TGGGCCCAGGTTTGAGG | N | 0.015400806 | 0.045332813 |
| | GAGAAGGTTGCAGAGCA | | | |
| | CTTCCCACCTCTCTGA | | | |
| 1280 | TGCACCCTGTAAGAATGG | N | 0.048100625 | 0.011586511 |
| | ACTTAAAAGTACTGCTGG | | | |
| | ACAGGCATGTGTGC | | | |
| 1281 | CACTACCAGGAGGAATA | P | 0.017829787 | 0.019574667 |
| | GCAACAGTCCGCCCTCT | | | |
| | CCGTCCTCTATGAACC | | | |
| 1282 | GCAATGTGGCCAGTAAG | N | 0.035873997 | 0.001447385 |
| | AGAACGTGAGTGGTAGC | | | |
| | ACTGAGGCGTGTCTGG | | | |
| 1283 | GCTAAATGTGAGGGTGG | N | 0.021782729 | 0.001811422 |
| | GCCCTAATAAGTACAAGT | | | |
| | GAGGACGAAGGCCGG | | | |
| 1284 | TTGCAGTGGTGGATTGAG | P | 0.029905802 | 0.000547326 |
| | AAGTCCAGTTTGATTTCA | | | |
| | TTGGGACAGACTGC | | | |
| 1285 | TCACTGATCTACAGCCCC | N | 0.02325871 | 0.038866497 |
| | TGTTCGGCGTCAGAGTC | | | |
| | CCCACTAGACCCAGT | | | |
| 1286 | CCCCATGTGCTGCTGAGT | N | 0.016912202 | 0.00094164 |
| | GGCCAAGATGATGCCAG | | | |
| | GCTGCCCTATACACT | | | |
| 1287 | TAATCTGGACATTCGAGG | N | 0.031154979 | 0.023087263 |
| | AATTGGCCGCTGTCACTG | | | |
| | CTTGTTGTTTGCGC | | | |
| 1288 | GGCCTTCCCTGTCAGAA | N | 0.007572616 | 0.009062624 |
| | GGGGGTTGTGGCAAAAG | | | |
| | CCACATTACAAGCTGC | | | |
| 1289 | CAAGCAGCCATACTGATG | N | 0.028082385 | 0.018091477 |
| | ACCACAGCAACCAGCTC | | | |
| | CACGGCCTGATGCAG | | | |
| 1290 | AGTCCCAGGACCAGACA | N | 0.020219021 | 0.023231981 |
| | TCCCCAGACTCCACAGAT | | | |
| | GTAATGAAGTCCCCG | | | |
| 1291 | TCCAGCCATGACAGAAG | P | 0.033488748 | 0.015580753 |
| | GACTTCTCCGTGCCCAAG | | | |
| | TGGATTCATCATTCC | | | |
| 1292 | TATGATGGGCGGCTACT | N | 0.037671037 | 0.000308618 |
| | GGAGGAGGCTGTGAGGA | | | |
| | AGAAGGGGTCGGAGGA | | | |
| 1293 | TTATGAAGGTGGCATTGG | N | 0.034511898 | 0.030408447 |
| | TGAGGCTGTGTCCAGTG | | | |
| | CAGTAGTGGGCGAGC | | | |
| 1294 | GCAAGAGCATTGACTGG | P | 0.041622014 | 0.041127826 |
| | GGCATCCTGGCTGTCTTC | | | |
| | ACCTGTGCTGAGAGC | | | |
| 1295 | GGCAGTTTCAACAGGGT | N | 0.042049007 | 0.023093844 |
| | GCTGCTGGCAACATGAAA | | | |
| | GGCATGATGGGATTC | | | |
| 1296 | CGCACGTTGCGGGCAGA | P | 0.039222975 | 0.012811036 |
| | GCGCAAGGCATACACCA | | | |
| | GAAAACGCTGTCCTGT | | | |
| 1297 | GCCAAAGAGGTCCACAA | P | 0.023127524 | 0.016144865 |
| | CCAGGTGTGCACTGTTCA | | | |
| | CTGCAGCCCATTTGC | | | |
| 1298 | GAGATTGCAAGGGCGGG | N | 0.045199009 | 0.000328698 |
| | GAGAGGAGGCTCTCAAT | | | |
| | AAATAATCGTGTAACC | | | |
| 1299 | TACCTACACAAGGCCACA | N | 0.046620525 | 0.000233283 |
| | CTCCTTGCGTTCCCCTTC | | | |
| | ACGGGATAAGGCGG | | | |
| 1300 | TCAGGCACATCATTGGAA | P | 0.009997869 | 0.000475435 |
| | TACAGGAAGTAGCCCTG | | | |
| | CACCTGCCAGTGAGC | | | |
| 1301 | GCCTCTTCCTCTGAATAG | P | 0.004795572 | 0.002757031 |
| | ACCAGACGCCCTTTCACT | | | |
| | TAGTTCAGTGCCAG | | | |
| 1302 | CCACACTTCCTACTTGGT | N | 0.04407875 | 0.028107594 |
| | CTCTGGAAGTTTTACCAC | | | |
| | ATGTAACAGATTCC | | | |
| 1303 | ATCCCGTAACAGCACTGT | N | 0.048578378 | 0.033340878 |
| | GGAATACACCCTTAACAA | | | |
| | GTTGGAGCCTGGCG | | | |
| 1304 | GGCTGGATGGACAGACA | P | 0.047285982 | 0.02699222 |
| | CCTCCCCCTACCCATATC | | | |
| | CCTCCCGTGTGTGGT | | | |
| 1305 | GGGCCCTGCCTTCAGCT | P | 0.004696064 | 0.001490776 |
| | GGTGCTTGCTGCGATTCC | | | |
| | TGTGCCTTATGTAAC | | | |
| 1306 | CGGCTTGCAAAACTTTCA | N | 0.047421646 | 0.033748406 |
| | GATGGAGTGGCTGTGCT | | | |
| | GAAGGTTGGTGGGAC | | | |
| 1307 | CAGGCATGCTACAATCCA | P | 0.026694516 | 0.005670334 |
| | GGACTGTGGTGTTCTATG | | | |
| | TGCCGTGTATGGTC | | | |
| 1308 | GGAATGGGAGAGGGGAA | P | 0.021591747 | 0.007443578 |
| | GTCTTGGCAGGGAAATC | | | |
| | CCTTTTGGCCACACAG | | | |
| 1309 | CTTGCAGGTTATTCTCGT | P | 0.015579306 | 0.007511241 |
| | CATCGTGTCCGCTTTCAG | | | |
| | TCGCATGGGCTGGA | | | |
| 1310 | CTGACACATCCTCTCTTT | N | 0.007769665 | 0.00103182 |
| | GCAAGCTGCTGACTGGG | | | |
| | CACACTCATGCCAAG | | | |
| 1311 | CCCTTGACCTCTTCTGGC | P | 0.027237423 | 0.018324409 |
| | ATTCTCCTGTGCTCTGAC | | | |
| | AAACTGAGCCAGCC | | | |
| 1312 | GAGGGTGGGCACACACC | N | 0.016389082 | 0.005154868 |
| | CAGCGGCCTGCAGAGTA | | | |
| | AGCTTATTACCCACAA | | | |
| 1313 | CCACTTGGAGAGCAAGA | N | 0.015301392 | 0.000164927 |
| | GGGCTTGTGGACTTGGG | | | |
| | GGAGCGGTTGTGAGAA | | | |
| 1314 | TGCAGCTGCTCCCCGGT | N | 0.041437114 | 0.005479412 |
| | GCACCCGCAACAGGCGT | | | |
| | TTTGCAATGCAGATGT | | | |
| 1315 | CCACCTTGGTTCGCCCAC | N | 0.044692603 | 0.000335386 |
| | TGCTGAACACCATCCAGA | | | |
| | AGACACAGAGGAGC | | | |
| 1316 | GTCAAACACTTGTGACTT | N | 0.028545026 | 0.022228659 |
| | TTGCTTTAATTCCATGAAT | | | |
| | GTTCCTGCCTCCT | | | |
| 1317 | ATCTAACTGTGTGTGGTA | N | 0.039113103 | 0.003001155 |
| | ACCTTGCGTCACGGAGC | | | |
| | TGTTAGTGAACGAGG | | | |
| 1318 | ATATCAACTGCCAGCCTG | N | 0.012971845 | 0.008485033 |
| | GAGAAGGTGACAGTCCA | | | |
| | AGTGTGCAACAGCTG | | | |
| 1319 | GGAGACGGGGGAGTTGA | N | 0.017213175 | 0.012700943 |
| | AGAGTGTGGCCCGCATT | | | |
| | GTCCTGGTTCCCAATA | | | |
| 1320 | GGGAACGGATGTGGAAG | P | 0.043070427 | 0.047486234 |
| | GAAGAACTGTCACCCTCT | | | |
| | TAAGGCCCAGGGTCG | | | |
| 1321 | CCCAGTCTTGTGGATGGA | P | 0.037145686 | 0.00324361 |
| | AATGTAGTGCTCGAGTCA | | | |
| | CATTCTGCTTAAAG | | | |
| 1322 | CGCTGCCTTGCGGGAGG | N | 0.048229484 | 0.001044857 |
| | GGGTCGAGAAAGAGGAA | | | |
| | CGAGGAGCTGTAAATA | | | |
| 1323 | AGGTTAAGCAAGAGCAAA | N | 0.017546081 | 0.00183069 |
| | GTGCCATTGTTTGCCTTT | | | |
| | AATTGGGGGGTGGG | | | |
| 1324 | ACCTGGGTGATGCTCCTT | N | 0.005738467 | 0.000742019 |
| | GGGGCCCTACCTAGAGG | | | |
| | GACTGACTTTTGTCC | | | |
| 1325 | TGGGGGTGGAGAAAGGG | N | 0.043175723 | 0.000606729 |
| | GAAGTGGTCCAGAAACAA | | | |
| | AAAGCCCCATTGGGC | | | |
| 1326 | CCCCCTTTGAATGAGGTC | P | 0.00802248 | 0.000613072 |
| | TTCCATGTTTGAGGGAAA | | | |
| | GTCTTGCACTATTG | | | |
| 1327 | CGGCCTGGGCTGAGAGG | P | 0.011578963 | 0.005431374 |
| | ATCAGTGACTTGTTTGCT | | | |
| | AGACACCGGACCAAA | | | |
| 1328 | ACTCAGGCTAGATATGAG | N | 0.029905474 | 0.000886558 |
| | GATATGTGGGGGGTCTC | | | |
| | AGCAGGAGCCTGGGG | | | |
| 1329 | GGCCCTCCCTGCACTCC | N | 0.024865447 | 0.004917457 |
| | CCTCTTGCTGCGTGTTGA | | | |
| | TTTGGAGGCACTGCA | | | |
| 1330 | CTGAGATTGAGCAACTGT | P | 0.03393354 | 0.023089639 |
| | ACTCCAGCTTGGCGACA | | | |
| | GAGCAAGACCCCCTC | | | |
| 1331 | GATCCCAACGAAGCAGC | P | 0.04685198 | 0.03278525 |
| | CCATGCGGTGACTGAAC | | | |
| | AGGCAATACAGTACGG | | | |
| 1332 | TTGGAGAAATGATGAGCT | N | 0.01099116 | 0.030807755 |
| | ACGCCTTGATGAAAGAAC | | | |
| | CGTGTTGGTGCTGC | | | |
| 1333 | TCATTCTCTGTCCAGCAG | P | 0.018675813 | 0.013857357 |
| | TCATGAACCCCTTCACCT | | | |
| | CCAATGACCTGATC | | | |
| 1334 | CAGGACAGAGGCAACGT | N | 0.023926688 | 0.033971053 |
| | GGAGAGGCTGAAAACAG | | | |
| | TGCAGAGACGTTTGAC | | | |
| 1335 | TTGCCGACCGAGGGGTG | P | 0.037289369 | 0.012997588 |
| | GACAACACTTTTGCAGAT | | | |
| | GAGCTGGTGGAGCTC | | | |
| 1336 | AAGAACCCTTGACCTGG | N | 0.015686307 | 0.006779361 |
| | GGCGTAATAAAGATGACC | | | |
| | TGGACCCCTGCCCCC | | | |
| 1337 | CCTGGGACAAGGACAAG | N | 0.042344756 | 0.00205149 |
| | GGAAAGGGATGGGTGAA | | | |
| | CCAGTAGGGAAGCTAG | | | |
| 1338 | CCCACCAATTTCTCGGAC | N | 0.048267569 | 0.011690512 |
| | ACTTCTCAGTGTGTGGAA | | | |
| | GCTCATGTGGGCCC | | | |
| 1339 | GATTCTGGAGTGAAGCA | P | 0.041369032 | 0.019763626 |
| | GATGTTACTCGACCTTTT | | | |
| | GTCTCCCAGGCTGTG | | | |
| 1340 | GGCCCTGGTTGAAAAGTA | P | 0.043521951 | 0.019369295 |
| | CTCATCTCCTGGTCTGAC | | | |
| | ATCCAAAGAGTCAC | | | |
| 1341 | GACATCGTCACCCTGCTA | P | 0.018313119 | 0.012789633 |
| | CGACTGGCAAAGATGAG | | | |
| | GGAGGCTGAAGCGGC | | | |
| 1342 | GCTCCAGTCGAGGAAGG | P | 0.024795772 | 0.014237616 |
| | AGTACGAGCAGGAGCTC | | | |
| | AGTGATGACTTGCATG | | | |
| 1343 | CCCCAAGGATATTTCCCT | P | 0.039643037 | 0.008061165 |
| | AACCTCACTCAGTCACAT | | | |
| | TGTAGGAGCCAGTG | | | |
| 1344 | GGCGGTGGAGGCCAATA | N | 0.033792671 | 0.006263419 |
| | CTTTGCAAAACCACGAAA | | | |
| | CCAAGTGGCTATGGC | | | |
| 1345 | TGGGAGACCCTCCAGGA | N | 0.025677362 | 0.035349218 |
| | CATTCCCACCCTCCCCCA | | | |
| | TGCTGCCAAGTTGTA | | | |
| 1346 | TCCAAGGCACAGTGAGC | N | 0.006739797 | 0.000202247 |
| | TGGGCAGAGCTGGGCTG | | | |
| | CCAGAAGCCTTTTTCA | | | |
| 1347 | TAAGTATTCCTGTCTCCA | P | 0.030606491 | 0.002722232 |
| | AAGGACCGGCTCTCCAT | | | |
| | GGCTCCTGCGCCTCG | | | |
| 1348 | CCCGTGGAGGTTGGCAT | N | 0.00562919 | 0.010891018 |
| | CCAGGTCACGCCAGAGG | | | |
| | ATGACGATTGTTTATC | | | |
| 1349 | CCATGATGAGGTAGCTTC | P | 0.016552143 | 0.009739356 |
| | TCCCTGGGCTCTCCTTCT | | | |
| | TGCCTGCCCTGTCT | | | |
| 1350 | GCTTTTGCTCAGTAACTG | N | 0.02488439 | 0.019402811 |
| | TGTCATGAATTGCAAGAG | | | |
| | TTTCCACAAACACT | | | |
| 1351 | CACCTTTAGCACGGATAG | P | 0.021921597 | 0.006080137 |
| | TTTCCTGGTCCCAAGTGG | | | |
| | GTGTGGAGCCTTCC | | | |
| 1352 | GAAGGGCAGACAGTTCTT | N | 0.004934525 | 0.000597018 |
| | CTGGGGTTGGCAGCTGC | | | |
| | TCATTCATGATGGCC | | | |
| 1353 | TCCTCTTGGGGTCCTTGA | N | 0.031665895 | 0.000389833 |
| | TGGGCATGTGTGATGGG | | | |
| | GAAGGAGCAGTCTCC | | | |
| 1354 | ACCCCACAGCTGCATTCA | N | 0.035332974 | 0.002266753 |
| | AACTCAAATCTGTGGGAA | | | |
| | TGAGTGACTCGACC | | | |
| 1355 | GTAGACACAGTCATCAGA | N | 0.040073926 | 0.006489628 |
| | AAATGTCTGCCCTTTTGT | | | |
| | TTACTTCTTGGTCC | | | |
| 1356 | GGGCTCTCTGAATCCTAC | P | 0.024620923 | 0.004493592 |
| | CTGGTTTCTTCAGGCTTC | | | |
| | TGGACTTGCTAGGC | | | |
| 1357 | TATCAGGTGCCACCATCA | N | 0.027321963 | 0.000897601 |
| | GTGCAGAGAAGCATTGG | | | |
| | GGGAACCAGTGGGCC | | | |
| 1358 | TTGGTGGCCTGCTTCCCT | P | 0.017927856 | 0.011451959 |
| | CATGCCCTGGAATACAAC | | | |
| | TCAGAGCTCCAGGC | | | |
| 1359 | CACCCCCGAGCTCGCAT | N | 0.047343085 | 0.006326286 |
| | GCTGTCACCCATTCCAGC | | | |
| | CTAAATGTGACCATA | | | |
| 1360 | TCTGAGCGGAAACCCTCT | P | 0.044691111 | 0.008485979 |
| | CCTTAATCTCACTGGCCC | | | |
| | ATCCCAGCCTCAGG | | | |
| 1361 | ATGGGGGGTGTGACCAA | N | 0.031334434 | 0.000199155 |
| | AATCAGTGGGATGTGGC | | | |
| | AGGAAGCTGCAGCCCA | | | |
| 1362 | CGCGAGCCCTGGTGTGG | P | 0.026435814 | 0.014793942 |
| | ACTGTGGTCTGTATGAAT | | | |
| | CGTGTGTAACTGTGG | | | |
| 1363 | TGGACCACTCCATTGCCA | N | 0.006547325 | 0.009124915 |
| | TCTACCTGCTCAACCCTG | | | |
| | ACGGCCTCTTCACG | | | |
| 1364 | GAAGACGGCATCACGAA | N | 0.0234632 | 0.005285233 |
| | GCAGCTCCAAAAGGAAAA | | | |
| | GCTTGGGCGGTGCCC | | | |
| 1365 | GGATTTGGTTAATGACTT | N | 0.041690557 | 0.007585438 |
| | ATGAGCAAGCTGGTTTGG | | | |
| | CCAGACAGTATACC | | | |
| 1366 | GGGGGCCCGTTCCCCAG | N | 0.041716203 | 0.000367849 |
| | AAGCTGCCAGTGCTTTCA | | | |
| | GATGCATTGACTCTT | | | |
| 1367 | AGCTGGCTGCTCAGACG | N | 0.03783101 | 0.000170023 |
| | GTCGACATTGAATTTGGG | | | |
| | TGGGGGTTGGGATCC | | | |
| 1368 | CACACATGCTTTTCTGCA | P | 0.041773378 | 0.027894609 |
| | CGTGGTTGCCTTAGTCAT | | | |
| | CTTCCTACAGCACC | | | |
| 1369 | AATGTGACAAAGGCTCGC | P | 0.015567544 | 0.016219853 |
| | ATAGCTGCTGGCTTACCA | | | |
| | ATGGCAGGGATACC | | | |
| 1370 | GGACAGCTGTTTTTTAAC | N | 0.046323685 | 0.011836486 |
| | CCTCTTCTGCAAGTTTGT | | | |
| | TGACCTACATGGGC | | | |
| 1371 | GACATCTCTAATGGAATC | N | 0.030534107 | 0.008581589 |
| | ATGGGGGAAACGGGTTG | | | |
| | GAATTTGTAGCCATG | | | |
| 1372 | TCTGTGAATCTTGGCTGG | N | 0.025159636 | 0.0116614 |
| | GACTTCCTCTGAGTGATG | | | |
| | CCTGAGGGTCAGCT | | | |
| 1373 | GCACCAACCAGTCCCGG | N | 0.008043655 | 0.01604216 |
| | GTTAGATCCCAAATGCTA | | | |
| | GAAGCCAGGGATGCC | | | |
| 1374 | GGCCAAGCCAGCAAAGC | N | 0.048020527 | 0.000175322 |
| | GGGAGCCCTGAAAAATTA | | | |
| | GGGGGGAAATGGGAG | | | |
| 1375 | ATCAAGAAGAAACTGAGC | N | 0.032832354 | 0.020524086 |
| | AAGGCCTGAGCGCTGCC | | | |
| | CTGCACCTCCGCAGA | | | |
| 1376 | GGGCCCAATTCTTCTCCA | P | 0.047201429 | 0.009196632 |
| | CGACAATGCCCGACCGC | | | |
| | ATGTTGCACAACCCA | | | |
| 1377 | GGCATATGCATATCCTGC | P | 0.030290378 | 0.005540374 |
| | TACTGCAGCTGCACCTAT | | | |
| | GATTGGTTATCCAA | | | |
| 1378 | GTACGTCCCACCCTGTCC | N | 0.044595708 | 0.004016554 |
| | CCAGATCCCCTATTCCCT | | | |
| | CCACAATAACAGAA | | | |
| 1379 | TGCTTTAGGTTTTTGAAA | P | 0.046323541 | 0.010093476 |
| | CAGCCCCGGCGACGCCT | | | |
| | CTATTGGCTCTCGGC | | | |
| 1380 | GCAGTAGAGTCTTGTTTA | P | 0.046085985 | 0.011158693 |
| | ATGGCATTTCACTGTTCA | | | |
| | TTCCCTTTACCACC | | | |
| 1381 | ACAAGCTGGAGCAGGCC | N | 0.019922786 | 0.019354296 |
| | AACGATGACGCGCGCAC | | | |
| | CTTCTACATCATCGAG | | | |
| 1382 | CACTGATGAATTTACCCT | P | 0.014225588 | 0.002703912 |
| | CAAGTTTCCTTCCTCTGT | | | |
| | ACCACTCTGCTTCC | | | |
| 1383 | TAGCGGGGATCCTGAAC | N | 0.048921321 | 0.002465521 |
| | TGGACTGAATAAAACGTG | | | |
| | GTCTCCCACTGCGCC | | | |
| 1384 | CGAGTGTGGCAGGTGAC | P | 0.043509997 | 0.002403266 |
| | CATTGGCACACGCTAGAA | | | |
| | GTTTATGGCAGAGCT | | | |
| 1385 | TGGTTGCCAGGAGACAG | N | 0.025141017 | 0.031947317 |
| | CAAGCAAAGCCAGCAGG | | | |
| | ACATGAAGTTGCTATT | | | |
| 1386 | AGTCCCTGCGGTCCCAG | N | 0.018985229 | 0.011950179 |
| | ATAGCCTGAATCCTGCCC | | | |
| | GGAGTGGAAGCTGAA | | | |
| 1387 | GGGCTGACTGAACCTAT | N | 0.028730784 | 0.022999352 |
| | GGCTAAGAATTGTGACAC | | | |
| | TCTCATGTTTCAAGC | | | |
| 1388 | AGGATCGTATCCCACACC | P | 0.018422828 | 0.031257716 |
| | AGGACTCTATCCTACTCC | | | |
| | TAGTAATCCTTTCT | | | |
| 1389 | GAAACTGTGGGGTTCATT | N | 0.049483463 | 0.003442174 |
| | AATTGCAGTGATGTGAAG | | | |
| | GGTCCTGACAGCAC | | | |
| 1390 | GATCCCTTCCGGGCTCT | N | 0.026408405 | 0.003350537 |
| | GGTCTATCTTGTCTCCTT | | | |
| | AGCTGGGGGCCTACA | | | |
| 1391 | CATGGGCAAAATCTAGCC | P | 0.037631963 | 0.038905 |
| | ACAGTCCTGAGAGTCCA | | | |
| | GGCTTCTGGGATGCC | | | |
| 1392 | GTCAAGTTGCCCAGCTTG | P | 0.011479622 | 0.00388929 |
| | GAGTTGTCTGTCACGCAC | | | |
| | ATGTGTCCTGTGGT | | | |
| 1393 | GACGACACCAGGTCTGA | N | 0.018611574 | 0.003706678 |
| | AGACTTGCGGCGTGAATT | | | |
| | TGGTCGTTATGGTCC | | | |
| 1394 | GGGAGTTTTGCCCTAACT | N | 0.018393593 | 0.017498695 |
| | CATGGATTGTGCAAGAAT | | | |
| | GAACTGCTGTTGGG | | | |
| 1395 | GGGAGGCTGAGGTGGGC | N | 0.005828462 | 0.001008454 |
| | ATATCACCTGAGGTCAGC | | | |
| | AGTTAAAGACCAGCC | | | |
| 1396 | GCAGACCTCCTTAGAGAC | P | 0.020624195 | 0.003890906 |
| | CTCCAGGCAGACCTCCTT | | | |
| | ACTGTCTTCAGGTG | | | |
| 1397 | TATAAGATATTAAAGGGT | P | 0.025930582 | 0.030983788 |
| | AAGTCTCTCCGGCCCGG | | | |
| | TTTCCCTCGGTGTGC | | | |
| 1398 | TTAAAACGCTCTCCCGAC | N | 0.00739715 | 0.001449154 |
| | TCGCCCACAGATTGAAAA | | | |
| | ATGCCTGCACAGCC | | | |
| 1399 | AGGACCAAACACCCCCA | N | 0.030010789 | 0.046182475 |
| | CCCCGATTGAGACCTGC | | | |
| | GGGTGCTGCTCTACAA | | | |
| 1400 | AGCCTGAGCTGCTGGAA | N | 0.032319331 | 0.004889254 |
| | ACTATTCCCTATGAATTC | | | |
| | ATGGCATAATAGGTG | | | |
| 1401 | TGGGATTCGGCCTCTGG | N | 0.004642484 | 0.001606086 |
| | AAAGTGGTGGTAGTTCCA | | | |
| | GATTTATGTGAATGT | | | |
| 1402 | CCTTTTTCTCCAGTATGC | P | 0.011676548 | 0.00174157 |
| | TACCTGATTTGTTTGGCT | | | |
| | GGCCACTAGGTGGC | | | |
| 1403 | CCACCCCTGCAGTCTCA | N | 0.049271074 | 0.037434275 |
| | GCTGTTTGGGAGGCTGA | | | |
| | AGCAAGAGGCTAGCTT | | | |
| 1404 | AGTGGGGGTGTGACCTG | N | 0.041738708 | 0.000934259 |
| | AGGAGATAAAGGAAGGA | | | |
| | CGACAGACCTGGGGAG | | | |
| 1405 | AACTTCCTACAGGGGCCA | N | 0.021215798 | 0.014217225 |
| | AAACCAGAGAAAGGCTTC | | | |
| | CAGCAACTTCGATG | | | |
| 1406 | GGAACAAGATGGTGATTC | P | 0.045428224 | 0.039061178 |
| | TGGGCAGGCAGTATGGG | | | |
| | TTTATTTTGCCAAAG | | | |
| 1407 | CTGAATGAGTACCGCCTC | P | 0.015605378 | 0.003521438 |
| | CCTAGGTTCCAGCACAG | | | |
| | CGCTCGGGTCTAAGA | | | |
| 1408 | CATATAAGCCCTGGGTCG | N | 0.038284887 | 0.007056442 |
| | GGGGGTAACTGTGGGGA | | | |
| | TCACTGCCTGAGACA | | | |
| 1409 | GCAGAGTGGCAGCAGAT | N | 0.031734622 | 0.024928665 |
| | GTTCCAGAAGGAGAAGG | | | |
| | CGGTGCTGGACGAGCT | | | |
| 1410 | CCTCTGTTTAACAGTCCT | P | 0.037354335 | 0.020840216 |
| | TGGACCATTCTGATCCAG | | | |
| | TTCACCAGTAGGTT | | | |
| 1411 | GGGAAGGGGCCCTTGGC | N | 0.036348985 | 0.007645893 |
| | CACAGGTGGAATTAAGAA | | | |
| | ATCTGGCGAAAAGCA | | | |
| 1412 | TGCACTCAGCCCTCCCA | P | 0.025582523 | 0.001475012 |
| | GCAAGGAGTCTGCCACT | | | |
| | CGCGCTTGAAGGACGT | | | |
| 1413 | ACAGAGCCCACAGCCCA | N | 0.030642087 | 0.002690855 |
| | TCTGCCTCTTCACCTCCC | | | |
| | TGAATCCGTGTCCAT | | | |
| 1414 | TCTGCCATTGCCTCTGTC | P | 0.013268571 | 0.001628801 |
| | TTCCTTGGGGCACCTCAG | | | |
| | CTCTGGATGCTACC | | | |
| 1415 | CTCACAGTGGCCATGGG | N | 0.039461062 | 0.00026877 |
| | GTGTCGGGGTGAAGGGC | | | |
| | TGTCCCAGCTACTTGT | | | |
| 1416 | ATACTTGAAACCTCTCTG | P | 0.040273242 | 0.003919337 |
| | ACCAAGAGCCTCTGATG | | | |
| | GAGTGGGAGGTGAGC | | | |
| 1417 | TGATTTGTGTTTTCAGTG | N | 0.018220008 | 1.71122E−05 |
| | TGTGGGGAAGCTGTCCT | | | |
| | GGGGGCTGGGGCGAC | | | |
| 1418 | CCCAGGGGGAGTACGGG | N | 0.038009216 | 0.000867431 |
| | GCTCAAAACACCCTTTTG | | | |
| | GAAAAACAAAGGTGG | | | |
| 1419 | CTAATAACAGTGACCTCC | P | 0.037407267 | 0.000361622 |
| | CCGCCAGGTCCTGTGTG | | | |
| | TTGCCGGCTGAAGAA | | | |
| 1420 | CATCTCTAACCAATCAGC | P | 0.007894577 | 0.006389395 |
| | ACTCCTGGCTCACTGGCT | | | |
| | TCCCCTCATCTGCC | | | |
| 1421 | ATCCCCCCTTCTGCAAGA | P | 0.004527991 | 0.000657809 |
| | AAGCCTCTTTGCAACTGG | | | |
| | GTCAGAATGGCGGC | | | |
| 1422 | CGATAGTAAATTATCCAT | N | 0.016961133 | 0.000666797 |
| | GCTGGTACCTGTGAAAGT | | | |
| | AAGCCCTGGGATCC | | | |
| 1423 | GTGGTAGATCACTTGAGG | P | 0.049858213 | 0.015040631 |
| | TCAAGAGTTGTGACACCA | | | |
| | GCCTGGCCAACCTG | | | |
| 1424 | TGGCTTGAACTCTTAGGG | P | 0.019261564 | 0.00379322 |
| | GTCTGCAGTGCTCCATCT | | | |
| | CCATTGGTGGCCCC | | | |
| 1425 | CGCCCCTATCCAGCAGG | P | 0.036235237 | 0.001955015 |
| | AAGCAGCCAGATGATCAA | | | |
| | CGACGCCCTTTTTCC | | | |
| 1426 | GAGGAGGGCCTCGTGGA | N | 0.047655844 | 0.001224815 |
| | GCGGCGGGAGGAGGCC | | | |
| | CAGCGGGCACAGCAGGC | | | |
| 1427 | CAGGCTCTGATTCTAGTG | N | 0.032491246 | 0.036993993 |
| | GAAGGAGCAGTTCTGAG | | | |
| | TCTCCAGGAAGTAGC | | | |
| 1428 | GGTGAGCTGAGATCACA | N | 0.047890481 | 0.007290033 |
| | CCACTGTATTCCAGCCTG | | | |
| | GGTGACTGAGACTCT | | | |
| 1429 | TCCAGCAGGTAGTGCGA | P | 0.012329027 | 0.007646791 |
| | GGAGATTCGGCAGCTTAT | | | |
| | TTCTGTGCCAGCTCC | | | |
| 1430 | GGCTTCTTGGCTTTCCTC | N | 0.047063144 | 0.000220076 |
| | TCTGACAAGTGACTGAAA | | | |
| | TGGGGGTAGGGTTG | | | |
| 1431 | GGGCCCAGATGTGGGTT | P | 0.038797351 | 0.039151904 |
| | TGCTCAGTATTAGTAGAC | | | |
| | AAGGTCTTTGTTCAG | | | |
| 1432 | ATGCTGGCCACTCTCAGT | P | 0.029588969 | 0.015218917 |
| | CCAGCGTTCCTCAGTAGT | | | |
| | GAATAGCGAACCTG | | | |
| 1433 | CCTGAGAGCATGAGGGC | N | 0.010027863 | 0.006149141 |
| | CCCTAGACAGAGTACAAG | | | |
| | GTGTAATTCAGACAG | | | |
| 1434 | CTGAGGCATCCTGCTGTC | N | 0.027217549 | 0.03359918 |
| | ATGGGAAGGTCTCCGCC | | | |
| | CAAATGTCAGATGCA | | | |
| 1435 | TGGGCTGGCTTGCCTTG | P | 0.036756065 | 0.014071165 |
| | GCTCTTTGAGCTCCCTTT | | | |
| | TGCTTAATTACTGGG | | | |
| 1436 | CAGTCCTTGGACCATTCT | P | 0.011793726 | 0.012790609 |
| | GATCCAGTTCACCAGTAG | | | |
| | GTTGGACAGCATAT | | | |
| 1437 | CCGGGCAGAGCAAGGAG | P | 0.048856132 | 0.011268539 |
| | AGAATGAGGAGTGACAT | | | |
| | GGGTGTTGCCGTGAGT | | | |
| 1438 | CAACTTAAGTTGAGTACA | N | 0.030218313 | 0.023287122 |
| | GTGGCTCAAGCCTGTAAT | | | |
| | CCTAGCACTTTAGG | | | |
| 1439 | CAGACAGTGTGGAGGGG | N | 0.010572541 | 0.004328754 |
| | CTGCTAAATGCCCTCAGG | | | |
| | TTCACTACAAAGCAC | | | |
| 1440 | TTGCGGACCTGGCGGAG | P | 0.033591176 | 0.034064875 |
| | ATGCGCGTTCGCTTCGAT | | | |
| | TGTAGGCCATTCCTT | | | |
| 1441 | GAATAGGGCAAGACTAAA | P | 0.010340898 | 0.014186685 |
| | GGACAGAGTAAGGGTGC | | | |
| | TGGCCGCCACCTGAC | | | |
| 1442 | GCTAGCTCCAGACATGG | P | 0.023688896 | 0.008342659 |
| | GTTGATCACCTAGAGGAG | | | |
| | CTCTGGCTAAGGCAC | | | |
| 1443 | GCCCTTCACTCCCACCTG | P | 0.048202664 | 0.006731128 |
| | CTGCCAAAGTCCCTGTGC | | | |
| | TAATGGGATTACAA | | | |
| 1444 | AGGCATTCTGAGGGGCA | N | 0.038053195 | 0.000602994 |
| | ACGTGGAGGAAGGGCCA | | | |
| | GGGATGCATGGGATTT | | | |
| 1445 | GCAGGCCAAGCAGGGCA | P | 0.04569696 | 0.018900596 |
| | ACCCCACACCCTTGACAT | | | |
| | AAAAGCATCTTGAAG | | | |
| 1446 | ATTGCCAAATACACTTTT | N | 0.018105402 | 0.007691746 |
| | CCAAATTTGTCCCAACAG | | | |
| | CCCTGTAAGCCAGC | | | |
| 1447 | AAACCCGTCACCCAGATC | P | 0.001192133 | 0.000342149 |
| | GTCAGCGCCGAGGCCTG | | | |
| | GGGTAGAGCAGGTGA | | | |
| 1448 | CTGGGGGTCTCCTGTGA | N | 0.023237758 | 0.000399927 |
| | CCCTGGAAAACTACCTCA | | | |
| | ATAGTCCTCGTAGCT | | | |
| 1449 | TTCGAGGGGCTACGGGT | N | 0.048906715 | 0.000216477 |
| | GGAGGGGCGAATGGAGG | | | |
| | AGCTCTCTGGGGCGAT | | | |
| 1450 | CTTCTGTCCCCCTTATGC | P | 0.041388797 | 0.002491223 |
| | CAACAAGATGGCCTTCCC | | | |
| | CTCTGAAACAAAGT | | | |
| 1451 | GCGGGGAAACTCCCAGT | N | 0.03413818 | 0.005387292 |
| | AGGCGCTGCTGTCCAGG | | | |
| | GAAAATGATGGGCTTT | | | |
| 1452 | CTTCCCTCTTGTCCCGAA | N | 0.013045093 | 0.023069059 |
| | GATCTGCGCCTCTAGTGC | | | |
| | CTTTTGAGGGGTTC | | | |
| 1453 | TTCAACACTGACCTGAGG | P | 0.011433165 | 0.027232951 |
| | TTTCAGAGCGCAGTCGTC | | | |
| | GGTGCGCTGCAGGA | | | |
| 1454 | TCCAGGCATTGTACTAAG | N | 0.024320051 | 0.000186095 |
| | TATGGGGAACCACAGAG | | | |
| | AAGACATTCCCTCAG | | | |
| 1455 | GTTTATCTGCTCTGATCA | N | 0.03941985 | 2.59256E−06 |
| | GGGGAAGGACAAACAGT | | | |
| | GGGGAGAGTCAGGGC | | | |
| 1456 | ATGAGCTACAGGAACAAG | P | 0.035980149 | 0.010332322 |
| | AGACCCAGTACTTCGGG | | | |
| | CCAGGCACGCGGCTC | | | |
| 1457 | ATGCATCATGAAGCTTCG | N | 0.047164938 | 0.003969276 |
| | AGTGAAGCTCTTCCTGGG | | | |
| | GACAATGTGGGACT | | | |
| 1458 | GGAAATGGGAGTGCTCA | N | 0.022818819 | 0.027570691 |
| | GTCTGTGCAAGTCAGAAT | | | |
| | CCTTGAAACTGGGCC | | | |
| 1459 | TGCCTGTGTAGTCTCTCT | N | 0.005297182 | 0.001019377 |
| | GTCGTTAGGCCTTTTATC | | | |
| | TATGCCTGTGTGTC | | | |
| 1460 | CTGCCGGAGCCCGAGTG | P | 0.043226163 | 0.035418441 |
| | GATCGGGGACGGAGAGA | | | |
| | ACACGAGCCCATCAGG | | | |
| 1461 | TTACGCCTCCTGATCCTT | N | 0.032137684 | 0.000101771 |
| | CCGATTGGGGCAGACTA | | | |
| | GGAGAGGAAAGACGG | | | |
| 1462 | TGCAGTGGGGACTTCGT | P | 0.022347237 | 0.000582218 |
| | GGGAGGCACTCATGGCT | | | |
| | CTCTGGGTCTAATGAA | | | |
| 1463 | CCAGGAGGCCGAACACT | P | 0.039300834 | 0.008361825 |
| | TCTTTCTGCTTTCTTGACA | | | |
| | TCCGCTCACCAGGC | | | |
| 1464 | AAACAACTGCCCATCCCG | N | 0.02252172 | 0.039673442 |
| | GGTCCTTTCCCTGATGGG | | | |
| | TTGTGGCAGTTACC | | | |
| 1465 | GTCTTCACATCTACCTTT | P | 0.038302577 | 0.008757631 |
| | CTATGTCACATGTGCAAG | | | |
| | ATGGTTGCTCTGCC | | | |
| 1466 | CTCATCAGCATCCAAGGG | N | 0.01483882 | 0.002765869 |
| | TGGGGAGCAGTGTCATC | | | |
| | TAGGAGACTGGTTCT | | | |
| 1467 | GTATGGAGTGGAAACGC | P | 0.046435225 | 0.013781452 |
| | TTGTAAGGCTTCACCAGG | | | |
| | ATCCACCTCTGATGT | | | |
| 1468 | CAGAGGGAGTTCACACA | P | 0.038174371 | 0.018150923 |
| | AGGAGTAAAGCCTGACT | | | |
| | GGACCATTGCACGGAT | | | |
| 1469 | GGTTGAGTTGTTACCGTT | N | 0.039955721 | 0.01184542 |
| | GAACTCACAGCCCACTG | | | |
| | GACTAGAACACATGC | | | |
| 1470 | CTGACCCTCTGCTGTTAT | P | 0.019419418 | 0.007603546 |
| | CCGGAAGTTTCTACCCG | | | |
| | GAGCCAGTTGCCTTC | | | |
| 1471 | GGTTTTGCGGCTAGTTGG | P | 0.039455 | 0.008206158 |
| | CTATTCAAGAAACCTCGC | | | |
| | CCCTCTGAATGTCA | | | |
| 1472 | GGAAGGTGTGATCTGTG | N | 0.0241199 | 0.033478154 |
| | GGACTGTCTGGGCCTGT | | | |
| | TACTCATCCTGCTATC | | | |
| 1473 | GTCCACAGTGAAGTCCAC | N | 0.011419633 | 0.013006478 |
| | TCCAGGTTATCTGCATAG | | | |
| | GTAGCCCAGGCACC | | | |
| 1474 | TGGAATAAGGAGGGGGA | N | 0.038264007 | 0.000699509 |
| | ACACAACTAGCAAGAACA | | | |
| | AAACATTGGGTTGGC | | | |
| 1475 | TGCAAACCTGTACTTCCA | P | 0.008454607 | 0.003803975 |
| | TGATGTGGGAAGGTGAG | | | |
| | GCTGCCAAGGAGAAG | | | |
| 1476 | GCTAATGGCGGGGACCT | P | 0.037397329 | 0.014133064 |
| | GGCACCTTTGCTCTGTGA | | | |
| | CTCTCCAGCTGTATG | | | |
| 1477 | TGTGTCCAGGCTACCTG | P | 0.026837177 | 0.003642709 |
| | GCTGTGCGGCACAGGTT | | | |
| | TAGCCCCAACATGACT | | | |
| 1478 | CGATGAGTGCGGGTAGG | P | 0.039418046 | 0.021477248 |
| | AGCCGTGAGGTGCTTCT | | | |
| | CTGCTGTGACAAACGA | | | |
| 1479 | GGGCGAGGTGGGCGCC | N | 0.035702689 | 2.14211E−05 |
| | GGCGAGAAGGCGGAGCC | | | |
| | CCCGGACAGCGTAGGTT | | | |
| 1480 | GGGAACAGTGAAGTGCA | N | 0.048787314 | 0.011786659 |
| | GCAGATGATGCTTCGAG | | | |
| | GGTGGCTTTGAGGCCA | | | |
| 1481 | GTCCGAGAGGGCAGATT | N | 0.030220956 | 0.007817742 |
| | GCCTGAGGTCAGGAGTT | | | |
| | CAAGACCAGCCTGGGC | | | |
| 1482 | CCCGTGTTTCCTGGACC | N | 0.031203443 | 0.030897646 |
| | GCGAATCAGTGTGTTGG | | | |
| | GCATCAGTGTTTTCTG | | | |
| 1483 | CCCCAGCCCTAGCCCTTT | N | 0.037598251 | 0.043895851 |
| | AGCCTTTCACCCTGTGCT | | | |
| | CTGGAAAGGCTACC | | | |
| 1484 | GCTCAGACACTCTACAGC | N | 0.021753531 | 0.001450169 |
| | TGAGAGTAGACACTTGTG | | | |
| | GGAGGAGTCTGCTA | | | |
| 1485 | gcatgtgtatgatgtgtgtgcgtcggac | P | 0.022570565 | 0.007594639 |
| | cgcttctaggctactaagtgtc | | | |
| 1486 | AGGCTGCGGTGTCTGCT | N | 0.018173556 | 0.007361403 |
| | GCTACTCTCCGAGCTTCG | | | |
| | CAATGCCGCCCAAGG | | | |
| 1487 | CTCAAGGCCAAGCTCAC | N | 0.012247632 | 0.008829978 |
| | CCCTCAAGTGCTCTCACA | | | |
| | CTCGGGACCTAATTC | | | |
| 1488 | GGGCACCCCTCAAACTC | N | 0.006386641 | 0.010414346 |
| | AGTCATGTGGTTCCAAAC | | | |
| | TACCCCATTCCCCAC | | | |
| 1489 | CACTACCACATTCCAGTC | N | 0.021438733 | 0.000138524 |
| | TTTTAAGTCCGCTGGGGG | | | |
| | CCGAACAGCAGTGC | | | |
| 1490 | GAGTGGCGGCACCTTTG | P | 0.048190161 | 0.011886772 |
| | GCACATTCATGGCCATTG | | | |
| | GGATGGGCATCCGAT | | | |
| 1491 | GAGCCACCATCTACTGG | P | 0.009598215 | 0.022580225 |
| | GTGCTTCTCCTGAGTGTG | | | |
| | ACAGTGTAGGCTGGG | | | |
| 1492 | ACAAGGCACCTGCATTCA | P | 0.002522633 | 0.00095494 |
| | CAGGCGGCCCTGAGCAC | | | |
| | CTGGGTTCTGACTTT | | | |
| 1493 | TTGCACGTGTGGTAAGCA | N | 0.017604944 | 0.015904613 |
| | TAGGCTTGAAGAGGTGG | | | |
| | GTAGGCAGGTACATG | | | |
| 1494 | GCGGCAGCTGACAGAAA | N | 0.037070829 | 0.000166873 |
| | TATATGGTGGTCTCTCTG | | | |
| | CAGGGGAGTTCCAGC | | | |
| 1495 | TTGCCAAAGTCCCGCTGC | P | 0.015821535 | 0.034938843 |
| | CCCTGGTGCCGCTGACC | | | |
| | AAACAGCAGTACCTA | | | |
| 1496 | AGCAGACTGAGGAGGGA | N | 0.026840655 | 0.021201906 |
| | GGAGACGAGGTTCTCTT | | | |
| | GGCATCACTTTCTCCC | | | |
| 1497 | ACAGCTTCCCAAGCCCCA | P | 0.03139876 | 0.005309371 |
| | TCAATAAAGCCCCTGTTC | | | |
| | ACGCTGCACTGGTG | | | |
| 1498 | CTGGGCTGTGGTATTTGG | N | 0.003942079 | 0.013674087 |
| | GTGATCTTTACATTCTTCA | | | |
| | GACTCATGTGTGT | | | |
| 1499 | TGGGGCTCAGGGCCTCT | P | 0.029939558 | 0.008207084 |
| | TTACCATGTGCAGTGACC | | | |
| | ATTTCTCAGAGCAGG | | | |
| 1500 | TGCAGCTGTAACTGCTCA | P | 0.012944388 | 0.007396462 |
| | CGCCAAAATGGCTGATG | | | |
| | GGGAGGCTGCTGCTT | | | |
| 1501 | TGGTGGGAGCTTGTGGA | P | 0.03260228 | 0.012670114 |
| | GTCGGATCACGTACCTGT | | | |
| | GCAGAAACCGCCTCT | | | |
| 1502 | GTCAACAGGCCGGGCAC | N | 0.036596245 | 0.010321418 |
| | GGTGGCTCAGTTATTTCA | | | |
| | TCCTCAGCTGGGCTT | | | |
| 1503 | CCTTCCACCCGGACACG | P | 0.039100742 | 0.000627893 |
| | CGCAAGCCGATGCACAG | | | |
| | AGAGTGTGGCTTCATT | | | |
| 1504 | TGCCCTGTTATCTCCTAG | P | 0.017791927 | 0.010808553 |
| | TGCTAACAATACACTCCA | | | |
| | GTCATGAGCCGGGC | | | |
| 1505 | AGCATCACCTTCGCACTG | N | 0.024307434 | 0.00473334 |
| | AAGAGTGGAGAGAGTCT | | | |
| | ACTGGATGACTGGCC | | | |
| 1506 | GGAGCCCCCTCTGGGTG | P | 0.047136485 | 0.033788195 |
| | GACTGCGCTTCTTCTCAA | | | |
| | CACAATACAAGGACG | | | |
| 1507 | GCCCACTTCGATGTGTTG | P | 0.031714559 | 0.000919257 |
| | TTCTTGTCGCACTTAGGG | | | |
| | ACATCAATCAAGGA | | | |
| 1508 | GTGCCCGCGATAGGCTT | P | 0.047426696 | 0.012338529 |
| | CTCTGGTTTTGTCTTTGC | | | |
| | TGTTCCTGGCAGCGC | | | |
| 1509 | GCTCACACATACAAAAAC | N | 0.020972604 | 0.004031424 |
| | TAAGTTGCCTTTCCTTGA | | | |
| | ATGAACCCTGTCTG | | | |
| 1510 | CTGCCAAACAGAGCAGT | N | 0.035426044 | 0.007870835 |
| | GGGTGAAATGGTCCCAG | | | |
| | GGTGACATGTTAGATC | | | |
| 1511 | AACCCCGCTCTGCCGCC | P | 0.02800267 | 0.025723563 |
| | GCTGCTGGAACCCATGA | | | |
| | GGCGTGCTTGCAGGCT | | | |
| 1512 | GCCTTTTTTAACTTGAGG | P | 0.025167703 | 0.004034367 |
| | GTGTAGAGGTCCTCCAC | | | |
| | GCTTGTTTGCCTGAA | | | |
| 1513 | GGACCAGCTACAAAATCT | N | 0.023945397 | 0.02021854 |
| | TCAGGACCAGCTCAGAAT | | | |
| | GAAAATGCCGGGGC | | | |
| 1514 | GGGCCGAGATGGTTTGT | N | 0.03801362 | 0.017926508 |
| | CTTATGCCTATAGCTGTT | | | |
| | TATGTCCCACCAGTG | | | |
| 1515 | GACTGCAGGCTCCCCTT | N | 0.014037978 | 0.003176608 |
| | CCTGCACCACCATTGTCT | | | |
| | CAGCAGTAAAGGCGA | | | |
| 1516 | GCCAGCCTTCCTGGTCC | P | 0.035725567 | 0.012929448 |
| | CGATTGTTCCATTAAGCT | | | |
| | TTATCCTCACCTACC | | | |
| 1517 | GATCTGCCGTCACTGGG | P | 0.045667657 | 0.002298658 |
| | GTGGCAACATCCTGGGT | | | |
| | CCTAAGTCTGTGGCTC | | | |
| 1518 | CGGCTGACGAGCTACCT | P | 0.008935471 | 0.022499083 |
| | TACTGAGCATATTCCTGC | | | |
| | CTCTACACCAGAGAC | | | |
| 1519 | AGCAGCTGTTAGGGTCA | N | 0.04087459 | 0.020135815 |
| | GCAATTTCTCCGGCCAAC | | | |
| | TGTAGGAGGACTTGG | | | |
| 1520 | GGTGTGTTTCTTTGCTGG | P | 0.032019281 | 0.01375169 |
| | TCACACCCATGATGGTGG | | | |
| | CTACTCTGAGGATC | | | |
| 1521 | TGTTTGATCCACCCCCTT | N | 0.006035439 | 0.023062449 |
| | CCCTGAAAATCCTGGGA | | | |
| | GGTTTTATTGCTGCC | | | |
| 1522 | TCCCTGGGGGTTGGGGG | N | 0.044940574 | 0.0002559 |
| | CTGTGTCTCTCCATCCCT | | | |
| | TAGTAAAAATACAGC | | | |
| 1523 | GCAGTATCCAGTGAGTGA | N | 0.040441431 | 0.001224611 |
| | AGAACACTTGACTGACTC | | | |
| | TTGGGCCACCTCTG | | | |
| 1524 | TGGCCCCCAAGAAGGGA | N | 0.039751456 | 0.006887156 |
| | GACAGTTCTGCTGAAGAA | | | |
| | CTCGAACTGGATACT | | | |
| 1525 | CCTTAAAGGCCAAGAAG | N | 0.034682482 | 0.007146317 |
| | GCAGTGTTGAAAGGTGTC | | | |
| | CGCAGCCACACGCAA | | | |
| 1526 | TCTCCCTTTCCCATCTTG | N | 0.016471269 | 0.01357284 |
| | ACTTCTGGAATAGCCCTT | | | |
| | GCCAAATACCAGGG | | | |
| 1527 | GTGGTGGGCCAAGAACA | P | 0.019429179 | 0.002546118 |
| | GCCAGGCCAAAACCATT | | | |
| | GCCACGGAGTCTTGTC | | | |
| 1528 | TTCACATTGGCCAAGTTA | N | 0.031347949 | 0.024530823 |
| | TCATGTCCATCCACACCA | | | |
| | AGCTGCAGAACAAG | | | |
| 1529 | CCTGAGGGCCAGGAGGC | N | 0.021315115 | 3.57724E−05 |
| | TGGGACCTGGTTTGGGC | | | |
| | CAGAACTTCTTCATAA | | | |
| 1530 | CCAAAGGCACAGGCCAA | P | 0.021292259 | 0.003470759 |
| | GTTGTAGCTTTGTCCCTT | | | |
| | GCCATCATGCCCAAC | | | |
| 1531 | GTGATTCCTGCAACTTGA | P | 0.034130667 | 0.022954044 |
| | CCTTCAGGCTGGGAGAG | | | |
| | GTGGAGAGCCATGCC | | | |
| 1532 | GGCTGACATTGGGACTC | P | 0.046820644 | 0.02433559 |
| | CCCTTGGATTATCTTTGT | | | |
| | ATCAGGAGGGCCTCA | | | |
| 1533 | GCAATCACTACCTTTCTC | P | 0.011179677 | 0.010634337 |
| | GTTTCATTTGTGTAACCA | | | |
| | TGCAGCATAGGCAC | | | |
| 1534 | GGAGCAGCAGATGGCTC | P | 0.008462421 | 0.002925456 |
| | TCGGTTCCAGGTGTGGG | | | |
| | ACTATGAGGAGGGAGA | | | |
| 1535 | ACGCCCGCAGTCCCTCA | N | 0.025340155 | 0.031897996 |
| | TCAGCAATTCCCAAGCTC | | | |
| | CAAAGCTCCCTGGAA | | | |
| 1536 | GGAGCTAAGCAGCCTTA | P | 0.007970077 | 0.002394201 |
| | GATAGCAGCAGAAGGCT | | | |
| | TTTTGGATTCTCCTCC | | | |
| 1537 | GAGGTCAGGAGTTCAAAA | N | 0.020686819 | 0.00982343 |
| | CCAGCCTGGCCAACATG | | | |
| | GCGAAATCCTGTCTG | | | |
| 1538 | CCTCAGCTGCGAGGTTT | P | 0.044045119 | 0.012424261 |
| | GGACCTTGAGGCTTGGA | | | |
| | TGAGGGAATTTTTCCC | | | |

TABLE 2
312 Positive and Negative Predictor Genes of GVHD Outcome and Exemplary Probes

| Index | ProbeID | Accession no. | Gene name | Symbol | Synonyms |
|---|---|---|---|---|---|
| 1 | 380575 | NM_000978.3 | ribosomal protein L23 | RPL23 | MGC117346; rpL17; |
| | | | (RPL23), mRNA. | | MGC111167; MGC72008 |
| 2 | 940398 | NM_006360.3 | eukaryotic translation | EIF3M | FLJ29030; GA17; hfl-B5; |
| | | | initiation factor 3, subunit M | | eIF3m; PCID1; B5 |
| | | | (EIF3M), mRNA. | | |
| 3 | 990315 | NM_030752.2 | t-complex 1 (TCP1), | TCP1 | TCP-1-alpha; CCT-alpha; |
| | | | transcript variant 1, mRNA. | | CCT1; D6S230E; CCTa |
| 4 | 1240136 | NM_199345.3 | phosphatidylinositol 4- | PI4KAP2 | FLJ44912; MGC31920 |
| | | | kinase, catalytic, alpha | | |
| | | | polypeptide pseudogene 2 | | |
| | | | (PI4KAP2), mRNA. | | |
| 5 | 1820482 | NM_004548.1 | NADH dehydrogenase | NDUFB10 | PDSW |
| | | | (ubiquinone) 1 beta | | |
| | | | subcomplex, 10, 22 kDa | | |
| | | | (NDUFB10), mRNA. | | |
| 6 | 1850288 | NM_014153.2 | zinc finger CCCH-type | ZC3H7A | HSPC055; ZC3HDC7; |
| | | | containing 7A (ZC3H7A), | | ZC3H7; FLJ20318; |
| | | | mRNA. | | FLJ10027 |
| 7 | 2940022 | NM_000712.3 | biliverdin reductase A | BLVRA | BVRA; BLVR |
| | | | (BLVRA), mRNA. | | |
| 8 | 3370164 | NM_000701.6 | ATPase, Na+/K+ | ATP1A1 | MGC3285; MGC51750 |
| | | | transporting, alpha 1 | | |
| | | | polypeptide (ATP1A1), | | |
| | | | transcript variant 1, mRNA. | | |
| 9 | 3440400 | NM_020698.1 | transmembrane and coiled- | TMCC3 | KIAA1145 |
| | | | coil domain family 3 | | |
| | | | (TMCC3), mRNA. | | |
| 10 | 3450148 | NM_170734.2 | brain-derived neurotrophic | BDNF | MGC34632 |
| | | | factor (BDNF), transcript | | |
| | | | variant 6, mRNA. | | |
| 11 | 3780450 | NM_079837.2 | BTG3 associated nuclear | BANP | DKFZp761H172; |
| | | | protein (BANP), transcript | | FLJ10177; SMAR1; |
| | | | variant 2, mRNA. | | SMARBP1; FLJ20538 |
| 12 | 4200575 | NM_014232.1 | vesicle-associated | VAMP2 | SYB2; VAMP-2; FLJ11460 |
| | | | membrane protein 2 | | |
| | | | (synaptobrevin 2) (VAMP2), | | |
| | | | mRNA. | | |
| 13 | 4640689 | NM_001967.3 | eukaryotic translation | EIF4A2 | DDX2B; BM-010; EIF4A; |
| | | | initiation factor 4A, isoform | | EIF4F |
| | | | 2 (EIF4A2), mRNA. | | |
| 14 | 5220196 | NM_006565.2 | CCCTC-binding factor (zinc | CTCF | — |
| | | | finger protein) (CTCF), | | |
| | | | mRNA. | | |
| 15 | 5870632 | NM_004800.1 | transmembrane 9 | TM9SF2 | P76; MGC117391; |
| | | | superfamily member 2 | | FLJ26287 |
| | | | (TM9SF2), mRNA. | | |
| 16 | 6290392 | NM_005839.3 | serine/arginine repetitive | SRRM1 | SRM160; 160-KD; |
| | | | matrix 1 (SRRM1), mRNA. | | POP101; MGC39488 |
| 17 | 6380008 | NM_025209.2 | enhancer of polycomb | EPC1 | Epl1; DKFZp781P2312 |
| | | | homolog 1 (Drosophila) | | |
| | | | (EPC1), mRNA. | | |
| 18 | 6380427 | NM_202468.1 | GIPC PDZ domain | GIPC1 | IIP-1; TIP-2; GLUT1CBP; |
| | | | containing family, member 1 | | C19orf3; RGS19IP1; |
| | | | (GIPC1), transcript variant | | Hs.6454; SYNECTIIN; |
| | | | 3, mRNA. | | MGC15889; NIP; |
| | | | | | MGC3774; SEMCAP; GIPC |
| 19 | 6580553 | NM_005688.2 | ATP-binding cassette, sub- | ABCC5 | MOAT-C; pABC11; ABC33; |
| | | | family C (CFTR/MRP), | | MRP5; SMRP; EST277145; |
| | | | member 5 (ABCC5), | | DKFZp686C1782; MOATC |
| | | | transcript variant 1, mRNA. | | |
| 20 | 7210128 | NM_024408.2 | Notch homolog 2 | NOTCH2 | hN2; AGS2 |
| | | | (Drosophila) (NOTCH2), | | |
| | | | mRNA. | | |
| 21 | 10504 | NM_031950.2 | fibroblast growth factor | FGFBP2 | KSP37 |
| | | | binding protein 2 (FGFBP2), | | |
| | | | mRNA. | | |
| 22 | 20010 | NM_001014438.1 | cysteinyl-tRNA synthetase | CARS | CARS1; CYSRS; |
| | | | (CARS), transcript variant 4, | | MGC: 11246 |
| | | | mRNA. | | |
| 23 | 20056 | NM_003295.1 | tumor protein, | TPT1 | TCTP; p02; HRF; |
| | | | translationally-controlled 1 | | FLJ27337 |
| | | | (TPT1), mRNA. | | |
| 24 | 60053 | NM_000975.2 | ribosomal protein L11 | RPL11 | GIG34 |
| | | | (RPL11), mRNA. | | |
| 25 | 60397 | NR_001449.1 | tRNA lysine 1 (TRK1) on | TRK1 | — |
| | | | chromosome 17. | | |
| 26 | 70008 | NM_000433.2 | neutrophil cytosolic factor 2 | NCF2 | p67phox; NOXA2; P67- |
| | | | (65 kDa, chronic | | PHOX |
| | | | granulomatous disease, | | |
| | | | autosomal 2) (NCF2), | | |
| | | | mRNA. | | |
| 27 | 270544 | NM_003297.1 | nuclear receptor subfamily | NR2C1 | TR2-11; TR2 |
| | | | 2, group C, member 1 | | |
| | | | (NR2C1), transcript variant | | |
| | | | 1, mRNA. | | |
| 28 | 450195 | NM_001788.4 | septin 7 (SEPT7), transcript | SEPT7 | Nbla02942; CDC10; |
| | | | variant 1, mRNA. | | SEPT7A; CDC3 |
| 29 | 450431 | NM_199424.1 | WW domain containing E3 | WWP2 | WWp2-like; AIP2 |
| | | | ubiquitin protein ligase 2 | | |
| | | | (WWP2), transcript variant | | |
| | | | 2, mRNA. | | |
| 30 | 450615 | NM_005953.2 | metallothionein 2A (MT2A), | MT2A | MT2 |
| | | | mRNA. | | |
| 31 | 450762 | NM_021642.2 | Fc fragment of IgG, low | FCGR2A | FCGR2A1; CDw32; |
| | | | affinity IIa, receptor (CD32) | | CD32A; CD32; FcGR; |
| | | | (FCGR2A), mRNA. | | FCG2; IGFR2; FCGR2; |
| | | | | | MGC30032; MGC23887 |
| 32 | 460411 | NM_006390.2 | importin 8 (IPO8), mRNA. | IPO8 | FLJ26580; RANBP8 |
| 33 | 460750 | NM_024065.3 | phosducin-like 3 (PDCL3), | PDCL3 | VIAF1; HTPHLP; |
| | | | mRNA. | | MGC3062 |
| 34 | 520133 | NM_001005849.1 | SMT3 suppressor of mif two | SUMO2 | SMT3H2; HSMT3; |
| | | | 3 homolog 2 (S. cerevisiae) | | MGC117191; SMT3B |
| | | | (SUMO2), transcript variant | | |
| | | | 2, mRNA. | | |
| 35 | 520392 | NM_023914.2 | purinergic receptor P2Y, G- | P2RY13 | P2Y13; FKSG77; SP174; |
| | | | protein coupled, 13 | | GPR94; GPCR1; GPR86 |
| | | | (P2RY13), transcript variant | | |
| | | | 1, mRNA. | | |
| 36 | 580255 | NM_001624.2 | absent in melanoma 1 | AIM1 | ST4 |
| | | | (AIM1), mRNA. | | |
| 37 | 610014 | NM_003541.2 | histone cluster 1, H4k | HIST1H4K | dJ160A22.1; H4/d; H4FD; |
| | | | (HIST1H4K), mRNA. | | H4F2iii |
| 38 | 610309 | NM_207115.1 | zinc finger protein 580 | ZNF580 | — |
| | | | (ZNF580), transcript variant | | |
| | | | 2, mRNA. | | |
| 39 | 610670 | NM_145805.1 | ISL LIM homeobox 2 | ISL2 | FLJ10160 |
| | | | (ISL2), mRNA. | | |
| 40 | 620047 | NM_004331.2 | BCL2/adenovirus E1B | BNIP3L | BNIP3a; NIX |
| | | | 19 kDa interacting protein 3- | | |
| | | | like (BNIP3L), mRNA. | | |
| 41 | 630403 | NM_005830.2 | mitochondrial ribosomal | MRPS31 | MRP-S31; IMOGN38 |
| | | | protein S31 (MRPS31), | | |
| | | | nuclear gene encoding | | |
| | | | mitochondrial protein, | | |
| | | | mRNA. | | |
| 42 | 630706 | NM_144653.3 | BTB (POZ) domain | BTBD14A | BTBD14; MGC23427 |
| | | | containing 14A (BTBD14A), | | |
| | | | mRNA. | | |
| 43 | 670255 | NM_001924.2 | growth arrest and DNA- | GADD45A | GADD45; DDIT1 |
| | | | damage-inducible, alpha | | |
| | | | (GADD45A), mRNA. | | |
| 44 | 780603 | NR_002305.1 | protein disulfide isomerase | PDIA3P | ERp60; GRP58P |
| | | | family A, member 3 | | |
| | | | pseudogene (PDIA3P) on | | |
| | | | chromosome 1. | | |
| 45 | 830041 | NM_001005193.1 | olfactory receptor, family 7, | OR7G2 | OST260; OR19-6 |
| | | | subfamily G, member 2 | | |
| | | | (OR7G2), mRNA. | | |
| 46 | 830619 | NM_004083.4 | DNA-damage-inducible | DDIT3 | MGC4154; CEBPZ; |
| | | | transcript 3 (DDIT3), | | CHOP10; CHOP; |
| | | | mRNA. | | GADD153 |
| 47 | 870082 | NM_012402.2 | ADP-ribosylation factor | ARFIP2 | POR1 |
| | | | interacting protein 2 | | |
| | | | (arfaptin 2) (ARFIP2), | | |
| | | | mRNA. | | |
| 48 | 990056 | NM_020706.1 | splicing factor, | SFRS15 | SCAF4; DKFZP434E098; |
| | | | arginine/serine-rich 15 | | FLJ23364; SRA4; |
| | | | (SFRS15), mRNA. | | KIAA1172 |
| 49 | 990273 | NM_000998.4 | ribosomal protein L37a | RPL37A | MGC74786 |
| | | | (RPL37A), mRNA. | | |
| 50 | 990543 | NM_004768.2 | splicing factor, | SFRS11 | DKFZp686M13204; |
| | | | arginine/serine-rich 11 | | dJ677H15.2; p54 |
| | | | (SFRS11), mRNA. | | |
| 51 | 1030431 | NM_001995.2 | acyl-CoA synthetase long- | ACSL1 | FACL2; LACS; FACL1; |
| | | | chain family member 1 | | ACS1; LACS2; LACS1 |
| | | | (ACSL1), mRNA. | | |
| 52 | 1050408 | NM_005678.3 | SNRPN upstream reading | SNURF | — |
| | | | frame (SNURF), transcript | | |
| | | | variant 1, mRNA. | | |
| 53 | 1050762 | NM_003844.2 | tumor necrosis factor | TNFRSF10A | TRAILR1; MGC9365; |
| | | | receptor superfamily, | | APO2; DR4; CD261; |
| | | | member 10a | | TRAILR-1 |
| | | | (TNFRSF10A), mRNA. | | |
| 54 | 1070373 | NM_001012994.1 | sorting nexin family member | SNX30 | FLJ35589; FLJ46877; |
| | | | 30 (SNX30), mRNA. | | FLJ45069; FLJ26481; |
| | | | | | FLJ44686; FLJ34280 |
| 55 | 1070435 | NM_201433.1 | growth arrest-specific 7 | GAS7 | MGC1348; MLL/GAS7; |
| | | | (GAS7), transcript variant c, | | KIAA0394 |
| | | | mRNA. | | |
| 56 | 1070593 | NM_007246.2 | kelch-like 2, Mayven | KLHL2 | ABP-KELCH; MAV; |
| | | | (Drosophila) (KLHL2), | | MAYVEN |
| | | | mRNA. | | |
| 57 | 1090474 | NM_000073.1 | CD3g molecule, gamma | CD3G | MGC138597; CD3- |
| | | | (CD3-TCR complex) | | GAMMA; T3G |
| | | | (CD3G), mRNA. | | |
| 58 | 1170300 | NM_005950.1 | metallothionein 1G (MT1G), | MT1G | MT1; MT1K; MGC12386 |
| | | | mRNA. | | |
| 59 | 1170332 | NM_014911.3 | AP2 associated kinase 1 | AAK1 | DKFZp686K16132; |
| | | | (AAK1), mRNA. | | MGC164568; FLJ45252; |
| | | | | | FLJ23712; FLJ25931; |
| | | | | | KIAA1048; FLJ42882; |
| | | | | | DKFZp686F03202; |
| | | | | | MGC164570; FLJ31060; |
| | | | | | MGC138170 |
| 60 | 1230292 | NM_080651.1 | mediator complex subunit | MED30 | TRAP25; MGC9890; |
| | | | 30 (MED30), mRNA. | | MED30; THRAP6 |
| 61 | 1240064 | NM_012482.3 | zinc finger protein 281 | ZNF281 | FLJ12859; ZNP-99; ZBP- |
| | | | (ZNF281), mRNA. | | 99; FLJ14378 |
| 62 | 1240142 | NM_017654.2 | sterile alpha motif domain | SAMD9 | KIAA2004; C7orf5; OEF1; |
| | | | containing 9 (SAMD9), | | FLJ20073; NFTC; OEF2 |
| | | | mRNA. | | |
| 63 | 1240192 | NM_001319.5 | casein kinase 1, gamma 2 | CSNK1G2 | CK1g2 |
| | | | (CSNK1G2), mRNA. | | |
| 64 | 1260136 | NM_001080497.1 | multiple EGF-like-domains | MEGF9 | EGFL5 |
| | | | 9 (MEGF9), mRNA. | | |
| 65 | 1340537 | NM_001001655.1 | alkB, alkylation repair | ALKBH2 | ABH2; MGC90512; hABH2 |
| | | | homolog 2 (E. coli) | | |
| | | | (ALKBH2), mRNA. | | |
| 66 | 1410068 | NM_019884.2 | glycogen synthase kinase 3 | GSK3A | DKFZp686D0638 |
| | | | alpha (GSK3A), mRNA. | | |
| 67 | 1410168 | NM_001421.2 | E74-like factor 4 (ets | ELF4 | MEF; ELFR |
| | | | domain transcription factor) | | |
| | | | (ELF4), mRNA. | | |
| 68 | 1410221 | NM_005621.1 | S100 calcium binding | S100A12 | CAAF1; CAGC; ENRAGE; |
| | | | protein A12 (S100A12), | | p6; CGRP; MRP6 |
| | | | mRNA. | | |
| 69 | 1410411 | NM_182710.1 | HIV-1 Tat interacting | HTATIP | ESA1; TIP60; TIP; PLIP; |
| | | | protein, 60 kDa (HTATIP), | | HTATIP1; cPLA2 |
| | | | transcript variant 1, mRNA. | | |
| 70 | 1430347 | NM_001076785.1 | solute carrier family 7 | SLC7A6 | DKFZp686K15246; |
| | | | (cationic amino acid | | KIAA0245; LAT3; LAT-2; |
| | | | transporter, y+ system), | | y+LAT-2 |
| | | | member 6 (SLC7A6), | | |
| | | | transcript variant 2, mRNA. | | |
| 71 | 1440296 | NM_005324.3 | H3 histone, family 3B | H3F3B | H3F3A; H3.3B |
| | | | (H3.3B) (H3F3B), mRNA. | | |
| 72 | 1440747 | NM_003544.2 | histone cluster 1, H4b | HIST1H4B | H4FI; H4/I |
| | | | (HIST1H4B), mRNA. | | |
| 73 | 1470209 | NM_019026.2 | transmembrane and coiled- | TMCO1 | HP10122; TMCC4; RP11- |
| | | | coil domains 1 (TMCO1), | | 466F5.7; PCIA3; PNAS-136 |
| | | | mRNA. | | |
| 74 | 1510538 | NM_012307.2 | erythrocyte membrane | EPB41L3 | DAL-1; KIAA0987; 4.1B; |
| | | | protein band 4.1-like 3 | | FLJ37633; DAL1 |
| | | | (EPB41L3), mRNA. | | |
| 75 | 1570575 | NM_014574.3 | striatin, calmodulin binding | STRN3 | SG2NA |
| | | | protein 3 (STRN3), | | |
| | | | transcript variant 2, mRNA. | | |
| 76 | 1660687 | NM_001018089.1 | NMDA receptor regulated 2 | NARG2 | BRCC1 |
| | | | (NARG2), transcript variant | | |
| | | | 2, mRNA. | | |
| 77 | 1690189 | NM_152453.2 | transmembrane and coiled- | TMCO5 | MGC35118; FLJ35807 |
| | | | coil domains 5 (TMCO5), | | |
| | | | mRNA. | | |
| 78 | 1740220 | NM_004038.3 | amylase, alpha 1A | AMY1A | AMY1; AMY1B |
| | | | (salivary) (AMY1A), | | |
| | | | transcript variant 1, mRNA. | | |
| 79 | 1770609 | NM_198486.2 | ribosomal protein L7-like 1 | RPL7L1 | MGC62004; dJ475N16.4 |
| | | | (RPL7L1), mRNA. | | |
| 80 | 1780273 | XM_001127464.1 | PREDICTED: arachidonate | ALOX5 | — |
| | | | 5-lipoxygenase (ALOX5), | | |
| | | | mRNA. | | |
| 81 | 1780647 | NM_052853.3 | aarF domain containing | ADCK2 | MGC20727; AARF |
| | | | kinase 2 (ADCK2), mRNA. | | |
| 82 | 1820544 | NM_182679.1 | G patch domain containing | GPATCH4 | GPATC4 |
| | | | 4 (GPATCH4), transcript | | |
| | | | variant 2, mRNA. | | |
| 83 | 1940041 | NM_000631.3 | neutrophil cytosolic factor 4, | NCF4 | SH3PXD4; P40PHOX; |
| | | | 40 kDa (NCF4), transcript | | NCF; MGC3810 |
| | | | variant 1, mRNA. | | |
| 84 | 1940053 | NM_001681.2 | ATPase, Ca++ transporting, | ATP2A2 | DAR; ATP2B; MGC45367; |
| | | | cardiac muscle, slow twitch | | DD; SERCA2 |
| | | | 2 (ATP2A2), transcript | | |
| | | | variant 2, mRNA. | | |
| 85 | 1980594 | NR_002203.1 | ferritin, heavy polypeptide- | FTHL8 | — |
| | | | like 8 (FTHL8) on | | |
| | | | chromosome X. | | |
| 86 | 1990278 | NM_021642.2 | Fc fragment of IgG, low | FCGR2A | FCGR2A1; CDw32; |
| | | | affinity IIa, receptor (CD32) | | CD32A; CD32; FcGR; |
| | | | (FCGR2A), mRNA. | | FCG2; IGFR2; FCGR2; |
| | | | | | MGC30032; MGC23887 |
| 87 | 2000010 | NM_006231.2 | polymerase (DNA directed), | POLE | DKFZp434F222; FLJ21434; |
| | | | epsilon (POLE), mRNA. | | POLE1 |
| 88 | 2000048 | NM_173683.3 | XK, Kell blood group | XKR6 | C8orf7; XRG6; C8orf21 |
| | | | complex subunit-related | | |
| | | | family, member 6 (XKR6), | | |
| | | | transcript variant 2, mRNA. | | |
| 89 | 2030243 | NM_013393.1 | FtsJ homolog 2 (E. coli) | FTSJ2 | FJH1; DKFZp686J14194 |
| | | | (FTSJ2), mRNA. | | |
| 90 | 2060291 | NM_004099.4 | stomatin (STOM), transcript | STOM | EPB7; EPB72; BND7 |
| | | | variant 1, mRNA. | | |
| 91 | 2070288 | NM_175617.3 | metallothionein 1E (MT1E), | MT1E | MT1; MTD |
| | | | mRNA. | | |
| 92 | 2100196 | NM_005101.1 | ISG15 ubiquitin-like modifier | ISG15 | G1P2; UCRP; IFI15 |
| | | | (ISG15), mRNA. | | |
| 93 | 2100273 | NM_001402.5 | eukaryotic translation | EEF1A1 | EEF1A; FLJ25721; CCS-3; |
| | | | elongation factor 1 alpha 1 | | PTI1; CCS3; MGC102687; |
| | | | (EEF1A1), mRNA. | | MGC16224; EF-Tu; eEF1A- |
| | | | | | 1; EEF-1; MGC131894; |
| | | | | | HNGC:16303; GRAF-1EF; |
| | | | | | LENG7; EF1A |
| 94 | 2100292 | NM_002893.2 | retinoblastoma binding | RBBP7 | RbAp46; MGC138867; |
| | | | protein 7 (RBBP7), mRNA. | | MGC138868 |
| 95 | 2140753 | NM_001034996.1 | ribosomal protein L14 | RPL14 | CAG-ISL-7; CTG-B33; L14; |
| | | | (RPL14), transcript variant | | MGC88594; RL14; hRL14 |
| | | | 1, mRNA. | | |
| 96 | 2230678 | NM_001093.3 | acetyl-Coenzyme A | ACACB | ACC2; ACCB; HACC275 |
| | | | carboxylase beta (ACACB), | | |
| | | | mRNA. | | |
| 97 | 2320053 | NM_024632.4 | SAP30-like (SAP30L), | SAP30L | FLJ11526; NS4ATP2 |
| | | | mRNA. | | |
| 98 | 2320139 | NM_002954.3 | ribosomal protein S27a | RPS27A | UBCEP1; UBA80; CEP80; |
| | | | (RPS27A), mRNA. | | HUBCEP80; UBCEP80 |
| 99 | 2320653 | NM_018281.2 | enoyl Coenzyme A | ECHDC2 | FLJ10948 |
| | | | hydratase domain | | |
| | | | containing 2 (ECHDC2), | | |
| | | | mRNA. | | |
| 100 | 2340626 | NM_016020.1 | transcription factor B1, | TFB1M | CGI75; mtTFB; CGI-75 |
| | | | mitochondrial (TFB1M), | | |
| | | | mRNA. | | |
| 101 | 2350192 | NM_018694.2 | ADP-ribosylation-like factor | ARL6IP4 | MGC814; SRp25; SR-25 |
| | | | 6 interacting protein 4 | | |
| | | | (ARL6IP4), transcript | | |
| | | | variant 1, mRNA. | | |
| 102 | 2350563 | NM_005791.1 | M-phase phosphoprotein 10 | MPHOSPH10 | MPP10P; MPP10 |
| | | | (U3 small nucleolar | | |
| | | | ribonucleoprotein) | | |
| | | | (MPHOSPH10), mRNA. | | |
| 103 | 2360528 | NM_182776.1 | minichromosome | MCM7 | MCM2; CDC47; P1.1- |
| | | | maintenance complex | | MCM3; P1CDC47; |
| | | | component 7 (MCM7), | | CDABP0042; P85MCM; |
| | | | transcript variant 2, mRNA. | | PNAS-146 |
| 104 | 2450446 | NM_015906.3 | tripartite motif-containing 33 | TRIM33 | FLJ32925; TIF1G; RFG7; |
| | | | (TRIM33), transcript variant | | PTC7; TF1G; TIF1GAMMA; |
| | | | a, mRNA. | | TIFGAMMA |
| 105 | 2480037 | NM_178868.3 | CKLF-like MARVEL | CMTM8 | CKLFSF8; CKLFSF8-V2 |
| | | | transmembrane domain | | |
| | | | containing 8 (CMTM8), | | |
| | | | mRNA. | | |
| 106 | 2480328 | NM_032361.1 | THO complex 3 (THOC3), | THOC3 | TEX1; MGC5469 |
| | | | mRNA. | | |
| 107 | 2480487 | NM_005819.4 | syntaxin 6 (STX6), mRNA. | STX6 | — |
| 108 | 2490333 | NM_207336.1 | zinc finger protein 467 | ZNF467 | EZI; Zfp467 |
| | | | (ZNF467), mRNA. | | |
| 109 | 2570100 | NM_019112.3 | ATP-binding cassette, sub- | ABCA7 | ABCA-SSN; ABCX; |
| | | | family A (ABC1), member 7 | | FLJ40025 |
| | | | (ABCA7), mRNA. | | |
| 110 | 2570288 | NM_015677.1 | SH3 domain containing, | SH3YL1 | FLJ39121; Ray; |
| | | | Ysc84-like 1 (S. cerevisiae) | | DKFZP586F1318 |
| | | | (SH3YL1), mRNA. | | |
| 111 | 2570328 | NM_021643.1 | tribbles homolog 2 | TRIB2 | TRB2; GS3955 |
| | | | (Drosophila) (TRIB2), | | |
| | | | mRNA. | | |
| 112 | 2600204 | NM_014016.2 | SAC1 suppressor of actin | SACM1L | KIAA0851; SAC1; |
| | | | mutations 1-like (yeast) | | DKFZp686A0231 |
| | | | (SACM1L), mRNA. | | |
| 113 | 2640541 | NM_006364.2 | Sec23 homolog A (S. cerevisiae) | SEC23A | CLSD; MGC26267 |
| | | | (SEC23A), | | |
| | | | mRNA. | | |
| 114 | 2640707 | XM_001130839.1 | PREDICTED: nuclear | NR1D2 | — |
| | | | receptor subfamily 1, group | | |
| | | | D, member 2 (NR1D2), | | |
| | | | mRNA. | | |
| 115 | 2680082 | NM_000971.3 | ribosomal protein L7 | RPL7 | MGC117326; humL7-1 |
| | | | (RPL7), mRNA. | | |
| 116 | 2690224 | NM_030980.1 | interferon stimulated | ISG20L2 | FLJ12671 |
| | | | exonuclease gene 20 kDa- | | |
| | | | like 2 (ISG20L2), mRNA. | | |
| 117 | 2710196 | NM_018428.2 | UTP6, small subunit (SSU) | UTP6 | HCA66; C17orf40 |
| | | | processome component, | | |
| | | | homolog (yeast) (UTP6), | | |
| | | | mRNA. | | |
| 118 | 2760537 | NM_175621.2 | metallothionein E (MTE), | MTE | MT1I |
| | | | mRNA. | | |
| 119 | 2970079 | NM_003274.3 | transmembrane protein 1 | TMEM1 | EHOC-1; MGC126777; |
| | | | (TMEM1), transcript variant | | EHOC1; GT334 |
| | | | 1, mRNA. | | |
| 120 | 2970594 | NM_138373.3 | myeloid-associated | MYADM | SB135 |
| | | | differentiation marker | | |
| | | | (MYADM), transcript variant | | |
| | | | 2, mRNA. | | |
| 121 | 3130600 | NM_007048.4 | butyrophilin, subfamily 3, | BTN3A1 | BT3.1; CD277; |
| | | | member A1 (BTN3A1), | | MGC141880; BTF5 |
| | | | mRNA. | | |
| 122 | 3140041 | NM_007237.3 | SP140 nuclear body protein | SP140 | MGC126440; LYSP100-B; |
| | | | (SP140), transcript variant | | LYSP100-A |
| | | | 1, mRNA. | | |
| 123 | 3170440 | NM_022893.2 | B-cell CLL/lymphoma 11A | BCL11A | BCL11A-L; CTIP1; |
| | | | (zinc finger protein) | | FLJ10173; EVI9; BCL11A- |
| | | | (BCL11A), transcript variant | | XL; BCL11A-S; FLJ34997; |
| | | | 1, mRNA. | | KIAA1809 |
| 124 | 3170451 | NM_024815.3 | nudix (nucleoside | NUDT18 | FLJ22494 |
| | | | diphosphate linked moiety | | |
| | | | X)-type motif 18 (NUDT18), | | |
| | | | mRNA. | | |
| 125 | 3180273 | NM_020315.4 | pyridoxal (pyridoxine, | PDXP | CIN; FLJ32703; PLP; |
| | | | vitamin B6) phosphatase | | dJ37E16.5 |
| | | | (PDXP), mRNA. | | |
| 126 | 3190133 | NR_002205.1 | ferritin, heavy polypeptide- | FTHL12 | — |
| | | | like 12 (FTHL12) on | | |
| | | | chromosome 9. | | |
| 127 | 3310546 | NM_001950.3 | E2F transcription factor 4, | E2F4 | E2F-4 |
| | | | p107/p130-binding (E2F4), | | |
| | | | mRNA. | | |
| 128 | 3370474 | NM_013368.2 | SERTA domain containing | SERTAD3 | RBT1 |
| | | | 3 (SERTAD3), transcript | | |
| | | | variant 1, mRNA. | | |
| 129 | 3450278 | NM_172232.1 | ATP-binding cassette, sub- | ABCA5 | FLJ16381; |
| | | | family A (ABC1), member 5 | | DKFZp779N2435; |
| | | | (ABCA5), transcript variant | | DKFZp451F117; |
| | | | 2, mRNA. | | EST90625; ABC13 |
| 130 | 3450463 | NM_183376.1 | arrestin domain containing | ARRDC4 | FLJ36045 |
| | | | 4 (ARRDC4), mRNA. | | |
| 131 | 3450537 | NM_032564.2 | diacylglycerol O- | DGAT2 | HMFN1045; |
| | | | acyltransferase homolog 2 | | DKFZp686A15125 |
| | | | (mouse) (DGAT2), mRNA. | | |
| 132 | 3520093 | NM_021070.2 | latent transforming growth | LTBP3 | FLJ44138; FLJ42533; |
| | | | factor beta binding protein 3 | | FLJ39893; LTBP-3; |
| | | | (LTBP3), mRNA. | | pp6425; FLJ33431; LTBP2; |
| | | | | | DKFZP586M2123 |
| 133 | 3520598 | NM_019858.1 | G protein-coupled receptor | GPR162 | GRCA; A-2 |
| | | | 162 (GPR162), transcript | | |
| | | | variant A-2, mRNA. | | |
| 134 | 3610630 | NM_016302.2 | cereblon (CRBN), mRNA. | CRBN | MGC27358; |
| | | | | | DKFZp781K0715; MRT2A |
| 135 | 3710735 | NM_153819.1 | RAS guanyl releasing | RASGRP2 | CDC25L; CALDAG-GEFI |
| | | | protein 2 (calcium and | | |
| | | | DAG-regulated) | | |
| | | | (RASGRP2), transcript | | |
| | | | variant 2, mRNA. | | |
| 136 | 3780544 | NM_016047.3 | splicing factor 3B, 14 kDa | SF3B14 | Ht006; SF3B14a; SAP14; |
| | | | subunit (SF3B14), mRNA. | | CGI-110; HSPC175; P14 |
| 137 | 3800576 | NM_080914.1 | asialoglycoprotein receptor | ASGR2 | L-H2; CLEC4H2; Hs.1259; |
| | | | 2 (ASGR2), transcript | | ASGP-R |
| | | | variant 3, mRNA. | | |
| 138 | 3830273 | NM_020202.2 | nitrilase family, member 2 | NIT2 | MGC111199 |
| | | | (NIT2), mRNA. | | |
| 139 | 3830653 | NM_006736.5 | DnaJ (Hsp40) homolog, | DNAJB2 | HSPF3; HSJ1 |
| | | | subfamily B, member 2 | | |
| | | | (DNAJB2), transcript variant | | |
| | | | 2, mRNA. | | |
| 140 | 3850059 | NM_005574.2 | LIM domain only 2 | LMO2 | TTG2; RBTN2; RBTNL1; |
| | | | (rhombotin-like 1) (LMO2), | | RHOM2 |
| | | | mRNA. | | |
| 141 | 3890689 | NM_198053.1 | CD247 molecule (CD247), | CD247 | CD3Q; CD3H; TCRZ; |
| | | | transcript variant 1, mRNA. | | CD3Z; CD3-ZETA |
| 142 | 3930133 | NM_199004.1 | arrestin, beta 2 (ARRB2), | ARRB2 | ARR2; ARB2; |
| | | | transcript variant 2, mRNA. | | DKFZp686L0365 |
| 143 | 3930392 | NM_001097577.1 | angiogenin, ribonuclease, | ANG | RNASE5; MGC22466; |
| | | | RNase A family, 5 (ANG), | | RNASE4; MGC71966 |
| | | | transcript variant 2, mRNA. | | |
| 144 | 3940138 | NM_001009944.1 | polycystic kidney disease 1 | PKD1 | PBP |
| | | | (autosomal dominant) | | |
| | | | (PKD1), transcript variant 1, | | |
| | | | mRNA. | | |
| 145 | 3940358 | NM_001003712.1 | oxysterol binding protein- | OSBPL8 | MSTP120; ORP8; MST120; |
| | | | like 8 (OSBPL8), transcript | | MGC126578; |
| | | | variant 2, mRNA. | | DKFZp686A11164; |
| | | | | | OSBP10; MGC133203 |
| 146 | 3990112 | NM_001042445.1 | calpastatin (CAST), | CAST | MGC9402; BS-17 |
| | | | transcript variant 11, | | |
| | | | mRNA. | | |
| 147 | 4010400 | NM_002480.1 | protein phosphatase 1, | PPP1R12A | MGC133042; MYPT1; MBS |
| | | | regulatory (inhibitor) subunit | | |
| | | | 12A (PPP1R12A), mRNA. | | |
| 148 | 4040088 | NM_152772.1 | t-complex 11 (mouse)-like 2 | TCP11L2 | MGC40368 |
| | | | (TCP11L2), mRNA. | | |
| 149 | 4120039 | NR_002200.1 | ferritin, heavy polypeptide- | FTHL2 | — |
| | | | like 2 (FTHL2) on | | |
| | | | chromosome 1. | | |
| 150 | 4120341 | NM_002208.4 | integrin, alpha E (antigen | ITGAE | HUMINAE; CD103; |
| | | | CD103, human mucosal | | MGC141996 |
| | | | lymphocyte antigen 1; alpha | | |
| | | | polypeptide) (ITGAE), | | |
| | | | mRNA. | | |
| 151 | 4150132 | NM_017514.2 | plexin A3 (PLXNA3), | PLXNA3 | XAP-6; HSSEXGENE; |
| | | | mRNA. | | PLEXIN-A3; PLXN4; SEX; |
| | | | | | PLXN3; 6.3 |
| 152 | 4200068 | NM_016553.3 | nucleoporin 62 kDa | NUP62 | FLJ43869; DKFZp547L134; |
| | | | (NUP62), transcript variant | | MGC841; p62; SNDI; IBSN; |
| | | | 2, mRNA. | | FLJ20822 |
| 153 | 4210465 | NM_006889.3 | CD86 molecule (CD86), | CD86 | B7-2; B70; LAB72; |
| | | | transcript variant 2, mRNA. | | MGC34413; CD28LG2 |
| 154 | 4220468 | NM_001001787.1 | ATPase, Na+/K+ | ATP1B1 | MGC1798; ATP1B |
| | | | transporting, beta 1 | | |
| | | | polypeptide (ATP1B1), | | |
| | | | transcript variant 2, mRNA. | | |
| 155 | 4220632 | XM_001133534.1 | PREDICTED: ATPase, | ATP1B3 | — |
| | | | Na+/K+ transporting, beta 3 | | |
| | | | polypeptide, transcript | | |
| | | | variant 2 (ATP1B3), mRNA. | | |
| 156 | 4220672 | NM_005949.2 | metallothionein 1F (MT1F), | MT1F | MT1; MGC32732 |
| | | | mRNA. | | |
| 157 | 4220731 | NM_000917.2 | procollagen-proline, 2- | P4HA1 | P4HA; 4-PH alpha-1 |
| | | | oxoglutarate 4-dioxygenase | | |
| | | | (proline 4-hydroxylase), | | |
| | | | alpha polypeptide I | | |
| | | | (P4HA1), transcript variant | | |
| | | | 1, mRNA. | | |
| 158 | 4230093 | NM_001171.3 | ATP-binding cassette, sub- | ABCC6 | MOATE; EST349056; ARA; |
| | | | family C (CFTR/MRP), | | PXE1; ABC34; PXE; MLP1; |
| | | | member 6 (ABCC6), | | MRP6 |
| | | | transcript variant 1, mRNA. | | |
| 159 | 4230097 | NM_002128.4 | high-mobility group box 1 | HMGB1 | DKFZp686A04236; HMG3; |
| | | | (HMGB1), mRNA. | | SBP-1; HMG1 |
| 160 | 4230619 | NM_012198.2 | grancalcin, EF-hand | GCA | GCL |
| | | | calcium binding protein | | |
| | | | (GCA), mRNA. | | |
| 161 | 4250768 | NM_004645.2 | coilin (COIL), mRNA. | COIL | p80-coilin; CLN80 |
| 162 | 4260221 | NM_145911.1 | zinc finger protein 23 (KOX | ZNF23 | KOX16; Zfp612; ZNF359; |
| | | | 16) (ZNF23), mRNA. | | ZNF612 |
| 163 | 4280162 | NM_024041.2 | sodium channel modifier 1 | SCNM1 | MGC3180 |
| | | | (SCNM1), mRNA. | | |
| 164 | 4390301 | NM_016113.3 | transient receptor potential | TRPV2 | VRL; VRL-1; MGC12549; |
| | | | cation channel, subfamily V, | | VRL1 |
| | | | member 2 (TRPV2), | | |
| | | | mRNA. | | |
| 165 | 4490242 | NM_006256.2 | protein kinase N2 (PKN2), | PKN2 | PRO2042; PAK2; Pak-2; |
| | | | mRNA. | | PRKCL2; MGC150606; |
| | | | | | MGC71074; PRK2 |
| 166 | 4640220 | NM_145113.1 | MYC associated factor X | MAX | MGC34679; MGC36767; |
| | | | (MAX), transcript variant 3, | | MGC11225; MGC10775; |
| | | | mRNA. | | orf1; MGC18164 |
| 167 | 4670601 | NM_022804.2 | SNRPN upstream reading | SNURF | — |
| | | | frame (SNURF), transcript | | |
| | | | variant 2, mRNA. | | |
| 168 | 4730148 | NM_004986.2 | kinectin 1 (kinesin receptor) | KTN1 | KIAA0004; MGC133337; |
| | | | (KTN1), transcript variant 4, | | MU-RMS-40.19; CG1; KNT |
| | | | mRNA. | | |
| 169 | 4730181 | NR_002205.1 | ferritin, heavy polypeptide- | FTHL12 | — |
| | | | like 12 (FTHL12) on | | |
| | | | chromosome 9. | | |
| 170 | 4760474 | NM_006000.1 | tubulin, alpha 4a (TUBA4A), | TUBA4A | TUBA1; H2-ALPHA; |
| | | | mRNA. | | FLJ30169 |
| 171 | 4780678 | NM_001079.3 | zeta-chain (TCR) | ZAP70 | FLJ17670; ZAP-70; TZK; |
| | | | associated protein kinase | | STD; FLJ17679; SRK |
| | | | 70 kDa (ZAP70), transcript | | |
| | | | variant 1, mRNA. | | |
| 172 | 4830113 | NM_016619.1 | placenta-specific 8 | PLAC8 | C15; onzin |
| | | | (PLAC8), mRNA. | | |
| 173 | 4850091 | NM_006331.5 | EMG1 nucleolar protein | EMG1 | Grcc2f; C2F; NEP1 |
| | | | homolog (S. cerevisiae) | | |
| | | | (EMG1), mRNA. | | |
| 174 | 4850327 | NM_016205.1 | platelet derived growth | PDGFC | SCDGF |
| | | | factor C (PDGFC), mRNA. | | |
| 175 | 4860209 | NM_173468.2 | MOB1, Mps One Binder | MOBKL1A | MOB4A; MGC33910; |
| | | | kinase activator-like 1A | | MATS2; Mob1B |
| | | | (yeast) (MOBKL1A), | | |
| | | | mRNA. | | |
| 176 | 4880215 | NM_001514.3 | general transcription factor | GTF2B | TFIIB; TF2B |
| | | | IIB (GTF2B), mRNA. | | |
| 177 | 4890722 | NM_006139.1 | CD28 molecule (CD28), | CD28 | Tp44; MGC138290 |
| | | | mRNA. | | |
| 178 | 4920347 | NM_016442.3 | endoplasmic reticulum | ERAP1 | APPILS; ALAP; PILSAP; |
| | | | aminopeptidase 1 (ERAP1), | | ERAP1; ERAAP; ARTS-1; |
| | | | transcript variant 1, mRNA. | | ERAAP1; KIAA0525; A- |
| | | | | | LAP; PILS-AP; ARTS1 |
| 179 | 5050156 | NM_004050.2 | BCL2-like 2 (BCL2L2), | BCL2L2 | KIAA0271; BCLW; BCL-W |
| | | | mRNA. | | |
| 180 | 5080246 | NM_003522.3 | histone cluster 1, H2bf | HIST1H2BF | H2B/g; H2BFG |
| | | | (HIST1H2BF), mRNA. | | |
| 181 | 5090288 | NM_171999.2 | sal-like 3 (Drosophila) | SALL3 | ZNF796 |
| | | | (SALL3), mRNA. | | |
| 182 | 5090307 | NM_153362.1 | protease, serine, 35 | PRSS35 | dJ223E3.1; MGC46520; |
| | | | (PRSS35), mRNA. | | C6orf158 |
| 183 | 5090397 | NM_206909.2 | pleckstrin and Sec7 domain | PSD3 | DKFZp761K1423; EFA6R; |
| | | | containing 3 (PSD3), | | HCA67 |
| | | | transcript variant 2, mRNA. | | |
| 184 | 5090450 | NM_004818.2 | DEAD (Asp-Glu-Ala-Asp) | DDX23 | U5-100K; prp28; PRPF28; |
| | | | box polypeptide 23 | | MGC8416 |
| | | | (DDX23), mRNA. | | |
| 185 | 5130750 | NM_002729.4 | hematopoietically | HHEX | HEX; PRH; PRHX; |
| | | | expressed homeobox | | HOX11L-PEN; HMPH |
| | | | (HHEX), mRNA. | | |
| 186 | 5270291 | NM_017811.3 | ubiquitin-conjugating | UBE2R2 | UBC3B; FLJ20419; |
| | | | enzyme E2R 2 (UBE2R2), | | MGC10481; CDC34B |
| | | | mRNA. | | |
| 187 | 5290369 | NM_032582.3 | ubiquitin specific peptidase | USP32 | USP10; NY-REN-60 |
| | | | 32 (USP32), mRNA. | | |
| 188 | 5290482 | NM_031943.1 | IFP38 (IFP38), mRNA. | IFP38 | — |
| 189 | 5360500 | NM_152246.1 | carnitine | CPT1B | CPT1-M; KIAA1670; M- |
| | | | palmitoyltransferase 1B | | CPT1 |
| | | | (muscle) (CPT1B), nuclear | | |
| | | | gene encoding | | |
| | | | mitochondrial protein, | | |
| | | | transcript variant 3, mRNA. | | |
| 190 | 5390433 | NM_030621.2 | Dicer1, Dcr-1 homolog | DICER1 | Dicer; HERNA; KIAA0928 |
| | | | (Drosophila) (DICER1), | | |
| | | | transcript variant 2, mRNA. | | |
| 191 | 5420575 | NM_001013251.1 | solute carrier family 3 | SLC3A2 | 4F2HC; CD98; 4F2; |
| | | | (activators of dibasic and | | CD98HC; 4T2HC; MDU1; |
| | | | neutral amino acid | | NACAE |
| | | | transport), member 2 | | |
| | | | (SLC3A2), transcript variant | | |
| | | | 6, mRNA. | | |
| 192 | 5490753 | NM_005467.2 | N-acetylated alpha-linked | NAALAD2 | MGC26353; MGC116996; |
| | | | acidic dipeptidase 2 | | NAALADASE2; |
| | | | (NAALAD2), mRNA. | | NAADALASE2 |
| 193 | 5550369 | NM_001125.2 | ADP-ribosylarginine | ADPRH | ARH1 |
| | | | hydrolase (ADPRH), | | |
| | | | mRNA. | | |
| 194 | 5670398 | NM_025191.2 | ER degradation enhancer, | EDEM3 | C1orf22 |
| | | | mannosidase alpha-like 3 | | |
| | | | (EDEM3), mRNA. | | |
| 195 | 5670682 | XM_943640.2 | PREDICTED: hypothetical | FLJ32255 | — |
| | | | protein LOC643977, | | |
| | | | transcript variant 2 | | |
| | | | (FLJ32255), mRNA. | | |
| 196 | 5810398 | NM_001080547.1 | spleen focus forming virus | SPI1 | SPI-A; OF; SFPI1; PU.1; |
| | | | (SFFV) proviral integration | | SPI-1 |
| | | | oncogene spi1 (SPI1), | | |
| | | | transcript variant 1, mRNA. | | |
| 197 | 5820068 | NM_032025.3 | eukaryotic translation | EIF2A | MSTP089; MSTP004; EIF- |
| | | | initiation factor 2A, 65 kDa | | 2A; CDA02; MST089 |
| | | | (EIF2A), mRNA. | | |
| 198 | 5820528 | NM_001077446.1 | tRNA splicing | TSEN34 | LENG5; SEN34; SEN34L |
| | | | endonuclease 34 homolog | | |
| | | | (S. cerevisiae) (TSEN34), | | |
| | | | transcript variant 2, mRNA. | | |
| 199 | 5860064 | NM_138782.1 | FCH domain only 2 | FCHO2 | — |
| | | | (FCHO2), mRNA. | | |
| 200 | 5870131 | NM_000492.3 | cystic fibrosis | CFTR | ABCC7; MRP7; TNR- |
| | | | transmembrane | | CFTR; CFTR/MRP; |
| | | | conductance regulator | | dJ760C5.1; CF; ABC35 |
| | | | (ATP-binding cassette sub- | | |
| | | | family C, member 7) | | |
| | | | (CFTR), mRNA. | | |
| 201 | 5890538 | NM_018708.2 | fem-1 homolog a (C. elegans) | FEM1A | DKFZp762M136; EPRAP |
| | | | (FEM1A), mRNA. | | |
| 202 | 5900112 | NM_052857.2 | coiled-coil domain | CCDC16 | MGC20398 |
| | | | containing 16 (CCDC16), | | |
| | | | mRNA. | | |
| 203 | 5910113 | NM_004385.2 | versican (VCAN), mRNA. | VCAN | DKFZp686K06110; WGN; |
| | | | | | VERSICAN; PG-M; WGN1; |
| | | | | | ERVR; CSPG2 |
| 204 | 6020327 | NM_024901.3 | DENN/MADD domain | DENND2D | FLJ22457; RP5-1180E21.2 |
| | | | containing 2D (DENND2D), | | |
| | | | mRNA. | | |
| 205 | 6020653 | NM_014962.2 | BTB (POZ) domain | BTBD3 | dJ742J24.1; MGC130038; |
| | | | containing 3 (BTBD3), | | KIAA0952; MGC130039 |
| | | | transcript variant 1, mRNA. | | |
| 206 | 6040487 | NM_006265.1 | RAD21 homolog (S. pombe) | RAD21 | KIAA0078; hHR21; NXP1; |
| | | | (RAD21), mRNA. | | FLJ40596; HRAD21; |
| | | | | | FLJ25655; SCC1; HR21; |
| | | | | | MCD1 |
| 207 | 6060196 | NM_145912.5 | NFAT activating protein with | NFAM1 | FLJ40652; CNAIP; |
| | | | ITAM motif 1 (NFAM1), | | bK126B4.4 |
| | | | mRNA. | | |
| 208 | 6110392 | NM_002076.2 | glucosamine (N-acetyl)-6- | GNS | G6S; MGC21274 |
| | | | sulfatase (Sanfilippo | | |
| | | | disease IIID) (GNS), | | |
| | | | mRNA. | | |
| 209 | 6180070 | NR_002204.1 | ferritin, heavy polypeptide- | FTHL11 | — |
| | | | like 11 (FTHL11) on | | |
| | | | chromosome 8. | | |
| 210 | 6180154 | NM_145255.2 | mitochondrial ribosomal | MRPL10 | MGC17973; MRP-L10; |
| | | | protein L10 (MRPL10), | | L10MT; RPML8; MRPL8; |
| | | | nuclear gene encoding | | MRP-L8 |
| | | | mitochondrial protein, | | |
| | | | mRNA. | | |
| 211 | 6180537 | NM_002139.2 | RNA binding motif protein, | RBMX | RBMXRT; HNRPG; |
| | | | X-linked (RBMX), mRNA. | | hnRNP-G; RNMX; |
| | | | | | RBMXP1 |
| 212 | 6200402 | NM_005946.2 | metallothionein 1A (MT1A), | MT1A | MTC; MT1; MGC32848; |
| | | | mRNA. | | MT1S |
| 213 | 6200669 | NM_138720.1 | histone cluster 1, H2bd | HIST1H2BD | H2B.1B; HIRIP2; |
| | | | (HIST1H2BD), transcript | | MGC90432; dJ221C16.6; |
| | | | variant 2, mRNA. | | H2B/b; H2BFB |
| 214 | 6290402 | NM_198723.1 | transcription elongation | TCEA2 | TFIIS |
| | | | factor A (SII), 2 (TCEA2), | | |
| | | | transcript variant 2, mRNA. | | |
| 215 | 6370025 | NM_013333.2 | epsin 1 (EPN1), mRNA. | EPN1 | — |
| 216 | 6370241 | NM_014155.3 | zinc finger and BTB domain | ZBTB44 | MGC57431; MGC60348; |
| | | | containing 44 (ZBTB44), | | BTBD15; MGC88058; |
| | | | mRNA. | | HSPC063; MGC26123 |
| 217 | 6380347 | NM_001469.3 | X-ray repair complementing | XRCC6 | TLAA; G22P1; CTCBF; |
| | | | defective repair in Chinese | | ML8; CTC75; KU70 |
| | | | hamster cells 6 (Ku | | |
| | | | autoantigen, 70 kDa) | | |
| | | | (XRCC6), mRNA. | | |
| 218 | 6380524 | NM_003432.1 | zinc finger protein 131 | ZNF131 | pHZ-10 |
| | | | (ZNF131), mRNA. | | |
| 219 | 6380639 | NM_213725.1 | ribosomal protein, large, P1 | RPLP1 | P1; FLJ27448; RPP1; |
| | | | (RPLP1), transcript variant | | MGC5215 |
| | | | 2, mRNA. | | |
| 220 | 6400148 | NM_080430.2 | selenoprotein M (SELM), | SELM | MGC40146; SEPM |
| | | | mRNA. | | |
| 221 | 6400332 | NM_014184.2 | cornichon homolog 4 | CNIH4 | HSPC163 |
| | | | (Drosophila) (CNIH4), | | |
| | | | mRNA. | | |
| 222 | 6400603 | NM_024070.3 | poliovirus receptor related | PVRIG | MGC138297; MGC2463; |
| | | | immunoglobulin domain | | MGC138295; MGC104322; |
| | | | containing (PVRIG), mRNA. | | C7orf15 |
| 223 | 6420730 | NM_001024921.2 | ribosomal protein L9 | RPL9 | FLJ27456; MGC15545; |
| | | | (RPL9), transcript variant 2, | | DKFZp313J1510; NPC-A- |
| | | | mRNA. | | 16 |
| 224 | 6480095 | NM_030918.5 | sorting nexin family member | SNX27 | MGC126873; MGC20471; |
| | | | 27 (SNX27), mRNA. | | MGC126871; MY014; |
| | | | | | KIAA0488 |
| 225 | 6550315 | NM_020424.2 | LYR motif containing 1 | LYRM1 | A211C6.1 |
| | | | (LYRM1), mRNA. | | |
| 226 | 6560121 | NM_002647.2 | phosphoinositide-3-kinase, | PIK3C3 | MGC61518; Vps34 |
| | | | class 3 (PIK3C3), mRNA. | | |
| 227 | 6560164 | NM_001006.3 | ribosomal protein S3A | RPS3A | FTE1; MGC23240; MFTL |
| | | | (RPS3A), mRNA. | | |
| 228 | 6580121 | NM_021242.3 | MID1 interacting protein 1 | MID1IP1 | THRSPL; MIG12; |
| | | | (gastrulation specific G12 | | STRAIT11499; FLJ10386; |
| | | | homolog (zebrafish)) | | G12-like |
| | | | (MID1IP1), mRNA. | | |
| 229 | 6620528 | NM_005952.2 | metallothionein 1X (MT1X), | MT1X | MT1; MT-1I |
| | | | mRNA. | | |
| 230 | 6620544 | NM_016360.2 | coiled-coil domain | CCDC44 | — |
| | | | containing 44 (CCDC44), | | |
| | | | mRNA. | | |
| 231 | 6650451 | NM_015057.3 | MYC binding protein 2 | MYCBP2 | FLJ21597; PAM; |
| | | | (MYCBP2), mRNA. | | FLJ13826; FLJ10106; |
| | | | | | FLJ21646; |
| | | | | | DKFZp686M08244; |
| | | | | | KIAA0916 |
| 232 | 6660162 | NM_052972.2 | leucine-rich alpha-2- | LRG1 | HMFT1766; LRG |
| | | | glycoprotein 1 (LRG1), | | |
| | | | mRNA. | | |
| 233 | 6760192 | NM_007236.3 | calcium binding protein P22 | CHP | SLC9A1BP |
| | | | (CHP), mRNA. | | |
| 234 | 6770634 | NM_005154.2 | ubiquitin specific peptidase | USP8 | KIAA0055; FLJ34456; |
| | | | 8 (USP8), mRNA. | | MGC129718; UBPY; |
| | | | | | HumORF8 |
| 235 | 6840020 | NM_006573.3 | tumor necrosis factor | TNFSF13B | TNFSF20; CD257; TALL1; |
| | | | (ligand) superfamily, | | delta BAFF; BAFF; ZTNF4; |
| | | | member 13b (TNFSF13B), | | TALL-1; THANK; BLYS |
| | | | mRNA. | | |
| 236 | 6900528 | NM_001033568.1 | ras homolog gene family, | RHOT1 | ARHT1; MIRO-1; |
| | | | member T1 (RHOT1), | | FLJ12633; FLJ11040 |
| | | | transcript variant 1, mRNA. | | |
| 237 | 6960593 | NM_004439.4 | EPH receptor AS (EPHA5), | EPHA5 | EHK1; TYRO4; HEK7; |
| | | | transcript variant 1, mRNA. | | CEK7 |
| 238 | 6960735 | NM_006004.1 | ubiquinol-cytochrome c | UQCRH | — |
| | | | reductase hinge protein | | |
| | | | (UQCRH), mRNA. | | |
| 239 | 6980092 | NM_024297.2 | PHD finger protein 23 | PHF23 | hJUNE-1b; MGC2941; |
| | | | (PHF23), mRNA. | | FLJ16355; FLJ22884 |
| 240 | 7000369 | NM_000591.2 | CD14 molecule (CD14), | CD14 | — |
| | | | transcript variant 1, mRNA. | | |
| 241 | 7000465 | NM_153615.1 | ral guanine nucleotide | RGL4 | Rgr; MGC119678; |
| | | | dissociation stimulator-like 4 | | MGC119680 |
| | | | (RGL4), mRNA. | | |
| 242 | 7050670 | NM_014649.2 | scaffold attachment factor | SAFB2 | KIAA0138 |
| | | | B2 (SAFB2), mRNA. | | |
| 243 | 7210035 | NR_003041.1 | small nucleolar RNA, C/D | SNORD13 | U13 |
| | | | box 13 (SNORD13) on | | |
| | | | chromosome 8. | | |
| 244 | 7210154 | NM_001165.3 | baculoviral IAP repeat- | BIRC3 | RNF49; MALT2; MIHC; |
| | | | containing 3 (BIRC3), | | HAIP1; API2; HIAP1; AIP1; |
| | | | transcript variant 1, mRNA. | | CIAP2 |
| 245 | 7210326 | NM_004159.4 | proteasome (prosome, | PSMB8 | D6S216; LMP7; RING10; |
| | | | macropain) subunit, beta | | MGC1491; D6S216E |
| | | | type, 8 (large multifunctional | | |
| | | | peptidase 7) (PSMB8), | | |
| | | | transcript variant 1, mRNA. | | |
| 246 | 7210450 | NM_006769.2 | LIM domain only 4 (LMO4), | LMO4 | — |
| | | | mRNA. | | |
| 247 | 7320041 | NM_015892.2 | B cell RAG associated | GALNAC4SNONE6ST | DKFZp781H1369; |
| | | | protein (GALNAC4S-6ST), | | KIAA0598; BRAG; RP11- |
| | | | mRNA. | | 47G11.1; MGC34346 |
| 248 | 7320551 | NM_002350.1 | v-yes-1 Yamaguchi | LYN | FLJ26625; JTK8 |
| | | | sarcoma viral related | | |
| | | | oncogene homolog (LYN), | | |
| | | | mRNA. | | |
| 249 | 7380255 | NM_022481.5 | centaurin, delta 3 | CENTD3 | FLJ21065; ARAP3; DRAG1 |
| | | | (CENTD3), mRNA. | | |
| 250 | 7400653 | NM_004567.2 | 6-phosphofructo-2- | PFKFB4 | — |
| | | | kinase/fructose-2,6- | | |
| | | | biphosphatase 4 (PFKFB4), | | |
| | | | mRNA. | | |
| 251 | 7400673 | NM_001039457.1 | ATPase, H+ transporting, | ATP6V0B | HATPL; ATP6F; VMA16 |
| | | | lysosomal 21 kDa, V0 | | |
| | | | subunit b (ATP6V0B), | | |
| | | | transcript variant 2, mRNA. | | |
| 252 | 7550364 | NM_001077628.1 | anterior pharynx defective 1 | APH1A | APH-1A; CGI-78; |
| | | | homolog A (C. elegans) | | 6530402N02Rik |
| | | | (APH1A), transcript variant | | |
| | | | 1, mRNA. | | |
| 253 | 7610187 | NM_182810.1 | activating transcription | ATF4 | TXREB; TAXREB67; |
| | | | factor 4 (tax-responsive | | CREB2; CREB-2 |
| | | | enhancer element B67) | | |
| | | | (ATF4), transcript variant 2, | | |
| | | | mRNA. | | |
| 254 | 7610537 | NM_002129.2 | high-mobility group box 2 | HMGB2 | HMG2 |
| | | | (HMGB2), mRNA. | | |
| 255 | 7650209 | NM_001003943.1 | Bcl2 modifying factor | BMF | FLJ00065 |
| | | | (BMF), transcript variant 4, | | |
| | | | mRNA. | | |
| 256 | 510132 | XM_941861.1 | PREDICTED: similar to | LOC650029 | — |
| | | | RNA-binding protein 4 | | |
| | | | (RNA-binding motif protein | | |
| | | | 4) (Lark homolog) (Hlark) | | |
| | | | (RNA-binding motif protein | | |
| | | | 4a), transcript variant 1 | | |
| | | | (LOC650029), mRNA. | | |
| 257 | 610280 | NM_025029.2 | family with sequence | FAM128B | FLJ14346; MGC87017 |
| | | | similarity 128, member B | | |
| | | | (FAM128B), mRNA. | | |
| 258 | 650129 | BX093310 | BX093310 | NONE | — |
| | | | NCI_CGAP_GC4 cDNA | | |
| | | | clone IMAGp998F143166; | | |
| | | | IMAGE: 1257997, mRNA | | |
| | | | sequence | | |
| 259 | 830484 | XM_938599.2 | PREDICTED: similar to 40S | LOC441377 | — |
| | | | ribosomal protein S26 | | |
| | | | (LOC441377), mRNA. | | |
| 260 | 830639 | XM_929667.1 | PREDICTED: similar to | LOC653778 | — |
| | | | solute carrier family 25, | | |
| | | | member 37 (LOC653778), | | |
| | | | mRNA. | | |
| 261 | 870181 | NM_001080544.1 | similar to ribosomal protein | LOC653314 | — |
| | | | L19 (LOC653314), mRNA. | | |
| 262 | 1010039 | AI218425 | qh24c08.x1 | NONE | — |
| | | | Soares NFL_T_GBC_S1 | | |
| | | | cDNA clone | | |
| | | | IMAGE: 1845614 3, mRNA | | |
| | | | sequence | | |
| 263 | 1260066 | AK024852 | cDNA: FLJ21199 fis, clone | NONE | — |
| | | | COL00235 | | |
| 264 | 1500538 | XM_928168.1 | PREDICTED: similar to | LOC645138 | — |
| | | | ribosomal protein S11 | | |
| | | | (LOC645138), mRNA. | | |
| 265 | 1940274 | NM_032036.2 | family with sequence | FAM14A | TLH29; MGC44913 |
| | | | similarity 14, member A | | |
| | | | (FAM14A), mRNA. | | |
| 266 | 1980112 | NM_197956.1 | chromosome 9 open | C9orf90 | KIAA1896; |
| | | | reading frame 90 (C9orf90), | | DKFZp762G199; RP11- |
| | | | mRNA. | | 379C10.2; bA379C10.2 |
| 267 | 2000564 | NM_001042475.1 | chromosome 6 open | C6orf204 | MGC131785; RP11- |
| | | | reading frame 204 | | 57K17.2; bA57K17.2; NY- |
| | | | (C6orf204), transcript | | BR-15 |
| | | | variant 1, mRNA. | | |
| 268 | 2260025 | XR_015514.1 | PREDICTED: similar to | LOC730746 | — |
| | | | Heterogeneous nuclear | | |
| | | | ribonucleoprotein A1 (Helix- | | |
| | | | destabilizing protein) | | |
| | | | (Single-strand RNA-binding | | |
| | | | protein) (hnRNP core | | |
| | | | protein A1) (HDP) | | |
| | | | (LOC730746), mRNA. | | |
| 269 | 2340446 | XM_942351.2 | PREDICTED: similar to | LOC652726 | — |
| | | | ankyrin repeat domain 36 | | |
| | | | (LOC652726), mRNA. | | |
| 270 | 2470240 | NM_004848.2 | chromosome 1 open | C1orf38 | ICB-1 |
| | | | reading frame 38 (C1orf38), | | |
| | | | transcript variant 1, mRNA. | | |
| 271 | 2510253 | NM_145306.2 | chromosome 10 open | C10orf35 | — |
| | | | reading frame 35 | | |
| | | | (C10orf35), mRNA. | | |
| 272 | 2810082 | NM_016470.6 | chromosome 20 open | C20orf111 | dJ1183I21.1; HSPC207; |
| | | | reading frame 111 | | Perit1 |
| | | | (C20orf111), mRNA. | | |
| 273 | 2940066 | XM_928429.1 | PREDICTED: similar to | LOC388275 | — |
| | | | Heterogeneous nuclear | | |
| | | | ribonucleoprotein A1 (Helix- | | |
| | | | destabilizing protein) | | |
| | | | (Single-strand binding | | |
| | | | protein) (hnRNP core | | |
| | | | protein A1) (HDP-1) | | |
| | | | (Topoisomerase-inhibitor | | |
| | | | suppressed) (LOC388275), | | |
| | | | mRNA. | | |
| 274 | 2940452 | AK056642 | cDNA FLJ32080 fis, clone | NONE | — |
| | | | OCBBF2000015 | | |
| 275 | 3190348 | XM_944816.1 | PREDICTED: similar to 60S | LOC440927 | — |
| | | | acidic ribosomal protein P1, | | |
| | | | transcript variant 4 | | |
| | | | (LOC440927), mRNA. | | |
| 276 | 3400709 | AK094914 | cDNA FLJ37595 fis, clone | NONE | — |
| | | | BRCOC2007864 | | |
| 277 | 3460014 | NM_016613.5 | chromosome 4 open | C4orf18 | AD021; DKFZp434L142; |
| | | | reading frame 18 (C4orf18), | | AD036; FLJ38155 |
| | | | transcript variant 2, mRNA. | | |
| 278 | 3780148 | NM_024067.2 | chromosome 7 open | C7orf26 | MGC2718 |
| | | | reading frame 26 (C7orf26), | | |
| | | | mRNA. | | |
| 279 | 3850411 | XM_933119.1 | PREDICTED: similar to NY- | LOC653316 | — |
| | | | REN-7 antigen, transcript | | |
| | | | variant 4 (LOC653316), | | |
| | | | mRNA. | | |
| 280 | 3870470 | XR_015809.1 | PREDICTED: similar to 40S | LOC728973 | — |
| | | | ribosomal protein S7 (S8) | | |
| | | | (LOC728973), mRNA. | | |
| 281 | 4060382 | XM_931996.1 | PREDICTED: similar to | LOC643035 | — |
| | | | CG33096-PB, isoform B, | | |
| | | | transcript variant 2 | | |
| | | | (LOC643035), mRNA. | | |
| 282 | 4480600 | NM_080757.1 | chromosome 20 open | C20orf127 | dJ614O4.6; MGC118948 |
| | | | reading frame 127 | | |
| | | | (C20orf127), mRNA. | | |
| 283 | 4610681 | XM_939687.2 | PREDICTED: similar to | LOC653658 | — |
| | | | ribosomal protein S23 | | |
| | | | (LOC653658), mRNA. | | |
| 284 | 4860341 | NM_145060.3 | chromosome 18 open | C18orf24 | MGC10200; Ska1 |
| | | | reading frame 24 | | |
| | | | (C18orf24), transcript | | |
| | | | variant 2, mRNA. | | |
| 285 | 5310681 | XM_933085.1 | PREDICTED: similar to cis- | LOC653344 | — |
| | | | Golgi matrix protein GM130, | | |
| | | | transcript variant 2 | | |
| | | | (LOC653344), mRNA. | | |
| 286 | 5340278 | XM_932991.1 | PREDICTED: hypothetical | LOC643977 | — |
| | | | protein LOC643977, | | |
| | | | transcript variant 1 | | |
| | | | (LOC643977), mRNA. | | |
| 287 | 5390685 | XM_928197.1 | PREDICTED: similar to 60S | LOC643433 | — |
| | | | ribosomal protein L29 (Cell | | |
| | | | surface heparin binding | | |
| | | | protein HIP), transcript | | |
| | | | variant 1 (LOC643433), | | |
| | | | mRNA. | | |
| 288 | 5420438 | NM_138471.1 | hypothetical protein | LOC144097 | DKFZp762N0114 |
| | | | BC007540 (LOC144097), | | |
| | | | mRNA. | | |
| 289 | 5420750 | XM_941125.1 | PREDICTED: similar to 60S | LOC649447 | — |
| | | | ribosomal protein L29 (Cell | | |
| | | | surface heparin binding | | |
| | | | protein HIP) (LOC649447), | | |
| | | | mRNA. | | |
| 290 | 5490603 | NM_001080831.1 | hCG1783417 | LOC401019 | — |
| | | | (LOC401019), mRNA. | | |
| 291 | 5820202 | XR_018325.1 | PREDICTED: similar to | LOC644131 | — |
| | | | chaperonin containing | | |
| | | | TCP1, subunit 8 (theta) | | |
| | | | (LOC644131), mRNA. | | |
| 292 | 5890615 | NM_001045478.1 | chromosome 1 open | C1orf200 | — |
| | | | reading frame 200 | | |
| | | | (C1orf200), mRNA. | | |
| 293 | 5960086 | BP873537 | BP873537 Sugano cDNA | NONE | — |
| | | | library, embryonal kidney | | |
| | | | cDNA clone HKR13896, | | |
| | | | mRNA sequence | | |
| 294 | 6020066 | XM_940333.2 | PREDICTED: similar to | LOC651202 | — |
| | | | large subunit ribosomal | | |
| | | | protein L36a (LOC651202), | | |
| | | | mRNA. | | |
| 295 | 6110195 | NM_153367.2 | chromosome 10 open | C10orf56 | FLJ90798 |
| | | | reading frame 56 | | |
| | | | (C10orf56), mRNA. | | |
| 296 | 6200706 | AA082988 | zn08b06.s1 Stratagene | NONE | — |
| | | | hNT neuron (#937233) | | |
| | | | cDNA clone IMAGE: 546803 | | |
| | | | 3, mRNA sequence | | |
| 297 | 6270307 | XM_930344.2 | PREDICTED: similar to 40S | LOC644934 | — |
| | | | ribosomal protein S26, | | |
| | | | transcript variant 1 | | |
| | | | (LOC644934), mRNA. | | |
| 298 | 6270605 | NR_003040.1 | ribosomal protein L23a | LOC649946 | — |
| | | | pseudogene (LOC649946) | | |
| | | | on chromosome 11. | | |
| 299 | 6280446 | XM_926370.1 | PREDICTED: similar to 40S | LOC642989 | — |
| | | | ribosomal protein S25 | | |
| | | | (LOC642989), mRNA. | | |
| 300 | 6280706 | XM_933956.1 | PREDICTED: similar to | LOC644162 | — |
| | | | septin 7, transcript variant 4 | | |
| | | | (LOC644162), mRNA. | | |
| 301 | 6370288 | XM_938283.2 | PREDICTED: chromosome | C17orf68 | — |
| | | | 17 open reading frame 68 | | |
| | | | (C17orf68), mRNA. | | |
| 302 | 6480092 | NM_024519.2 | family with sequence | FAM65A | FLJ13725; KIAA1930 |
| | | | similarity 65, member A | | |
| | | | (FAM65A), mRNA. | | |
| 303 | 6510753 | XM_936874.1 | PREDICTED: similar to 60S | LOC642210 | — |
| | | | ribosomal protein L32 | | |
| | | | (LOC642210), mRNA. | | |
| 304 | 6660753 | NM_017822.3 | chromosome 12 open | C12orf41 | FLJ20436; FLJ12670 |
| | | | reading frame 41 | | |
| | | | (C12orf41), mRNA. | | |
| 305 | 6760202 | NM_001014812.1 | family with sequence | FAM96A | FLJ22875 |
| | | | similarity 96, member A | | |
| | | | (FAM96A), transcript variant | | |
| | | | 2, mRNA. | | |
| 306 | 6840477 | CD640673 | AGENCOURT_14535501 | NONE | — |
| | | | NIH_MGC_191 cDNA | | |
| | | | clone IMAGE: 30415823 5, | | |
| | | | mRNA sequence | | |
| 307 | 6860162 | XM_498969.2 | PREDICTED: hypothetical | LOC441019 | — |
| | | | LOC441019 (LOC441019), | | |
| | | | mRNA. | | |
| 308 | 7160079 | NM_016623.3 | family with sequence | FAM49B | L1; DKFZp686B04128; BM- |
| | | | similarity 49, member B | | 009 |
| | | | (FAM49B), mRNA. | | |
| 309 | 7320707 | XM_939368.1 | PREDICTED: similar to | LOC654103 | — |
| | | | solute carrier family 25, | | |
| | | | member 37 (LOC654103), | | |
| | | | mRNA. | | |
| 310 | 7400689 | NM_017896.2 | chromosome 20 open | C20orf11 | TWA1 |
| | | | reading frame 11 | | |
| | | | (C20orf11), mRNA. | | |
| 311 | 7510543 | NM_017924.2 | chromosome 14 open | C14orf119 | MGC74723; FLJ20671 |
| | | | reading frame 119 | | |
| | | | (C14orf119), mRNA. | | |
| 312 | 7610608 | NM_001093763.1 | hCG31916 (LOC653702), | LOC653702 | — |
| | | | mRNA. | | |

| | | | Minimal | Minimal |
|---|---|---|---|---|
| | | | p-value | p-value |
| | | P or N | precision- | standard |
| | | predictor | weighted | heteroscedastic |
| Index | Probe sequence | gene | T-test | T-test |
| 1 | TCCAGCAGTGGTCATTCG | N | 0.009966 | 0.001089 |
| | ACAACGAAAGTCATACCGT | | | |
| | AGAAAAGATGGCG | | | |
| 2 | CAGACCCAGAGAAAAGTA | N | 0.013671 | 0.00275 |
| | GTTGTCAGTCATAGCACAC | | | |
| | ATCGGACATTTGG | | | |
| 3 | GCAATGGTAAACCTCGAG | N | 0.001814 | 9.08E−05 |
| | ACAACAAACAAGCAGGGG | | | |
| | TGTTTGAACCAACC | | | |
| 4 | GTGAGCCTGGGCCCTACA | P | 0.009399 | 0.000919 |
| | TGGATGTGGTCGTCTCCC | | | |
| | TGGTCACTATCATG | | | |
| 5 | CAGAGGCAGAGGATGCTG | N | 0.001628 | 0.000849 |
| | CAAGAGAGAAAAGCTGCA | | | |
| | AAAGAGGCCGCCGC | | | |
| 6 | GTTGGGGAAGAGGATAAG | N | 0.009361 | 5.94E−05 |
| | GTTATATCTAGGACAACTC | | | |
| | TTTGAGTTGGTCC | | | |
| 7 | CTGAGAAGGAACTGGCTG | N | 0.001468 | 0.000119 |
| | CTGAAAAGAAACGCATCCT | | | |
| | GCACTGCCTGGGG | | | |
| 8 | CGAAGTCAGAAAACTCATC | P | 0.006818 | 0.000591 |
| | ATCAGGCGACGCCCTGGC | | | |
| | GGCTGGGTGGAGA | | | |
| 9 | GGGCAAACCCAAAGATGG | N | 0.019615 | 1.54E−06 |
| | AAAGTGCTTGTTGGGTGG | | | |
| | GTAAGCACCACCTG | | | |
| 10 | ATGTACGTGGGGGATTCTT | N | 0.012771 | 1.75E−05 |
| | GACTCGGGTTAGTCTCTG | | | |
| | GGGATGCAGAGCC | | | |
| 11 | TTTCGTTTGAGTCCTGCTG | P | 0.012871 | 0.007059 |
| | TTGGTGTCGGAGCACGAG | | | |
| | GGGAGGCACGGTG | | | |
| 12 | GCCCAGAGAGAGCTGTCC | P | 0.001982 | 0.000689 |
| | TCTCATTGGGTGAACTGAT | | | |
| | TGAGGAAGGGTCT | | | |
| 13 | GGACCCTGTTGCTAAGCC | P | 0.004037 | 0.000412 |
| | CCAGCAAGCAATCCTAGG | | | |
| | TAGGGTTTAATCCC | | | |
| 14 | ATGTAGCAGAATGGCACC | P | 0.007622 | 0.000468 |
| | CAGACCACTGCCCACCAG | | | |
| | TGACGGACATGCAC | | | |
| 15 | CAGTGTGGTGAAGGTTGA | N | 0.004099 | 0.001709 |
| | CTGAAGAAGTCCAGTGTG | | | |
| | TCCAGTTAAAACAG | | | |
| 16 | CAACTTTCAGAGCCTCTTG | P | 0.007348 | 0.000539 |
| | TATTTGGAAGGCTGGAAG | | | |
| | GGCCCAGACTTTG | | | |
| 17 | ACACAGTAGCGATGGAGG | P | 0.008241 | 0.00013 |
| | TGACGTAGCTTCCTCCGA | | | |
| | GTGGAACTGCAGCC | | | |
| 18 | CCCTCCCTGTGGAGCCTG | P | 0.0106 | 0.003695 |
| | TTACCTCCGCATTTGACAC | | | |
| | GAGTCTGCTGTGA | | | |
| 19 | GTTTGGTGTGTTCCCGCAA | P | 0.030046 | 0.000792 |
| | ACCCCCTTTGTGCTGTGG | | | |
| | GGCTGGTAGCTCA | | | |
| 20 | AGCCATAGCTGGTGACAA | N | 0.015967 | 0.008984 |
| | ACAGATGGTTGCTCAGGG | | | |
| | ACAAGGTGCCTTCC | | | |
| 21 | GCGCCTTTCTCATCAGCTT | N | 0.008279 | 0.005652 |
| | CTTCCGAGGGTGACAGGT | | | |
| | GAAAGACCCCTAC | | | |
| 22 | CATGGAGGGCAAAGAGCT | N | 0.018391 | 0.000887 |
| | CAGCAAAGGGCAAGCCAA | | | |
| | GAAGCTGAAGAAGC | | | |
| 23 | CCAGATGGCATGGTTGCT | N | 0.000708 | 0.00047 |
| | CTATTGGACTACCGTGAG | | | |
| | GATGGTGTGACCCC | | | |
| 24 | GCATTGGGGCCAAACACA | P | 0.029415 | 0.015178 |
| | GAATCAGCAAAGAGGAGG | | | |
| | CCATGCGCTGGTTC | | | |
| 25 | GCATCAGACTTTTAATCTG | P | 0.021705 | 0.001461 |
| | AGGGTCCAGGGTTCAAGT | | | |
| | CCCTGTTCGGGCG | | | |
| 26 | GGGGAGAGGAAAAGTGGA | N | 0.02414 | 0.020459 |
| | TGGAAGTGTCTGGAAAGG | | | |
| | GCACGAGAGAGTCT | | | |
| 27 | TGCCAGAACACAAGACAC | N | 0.033189 | 0.006635 |
| | CAAATTGAACTCACTGCTT | | | |
| | TTGAGGCATCTGG | | | |
| 28 | AGCATCTTTCATATGGTAG | N | 0.014552 | 0.006742 |
| | GAACCAACAAGGAAACTTT | | | |
| | CCTTTAACTCCC | | | |
| 29 | TACCGCCTCCTCCCCGTC | N | 0.041007 | 3.68E−05 |
| | GCTCTGCCTTTTCCAAAAC | | | |
| | TCACTTGGGCCCT | | | |
| 30 | GAACCCGCGTGCAACCTG | P | 0.015933 | 0.019757 |
| | TCCCGACTCTAGCCGCCT | | | |
| | CTTCAGCACGCCAT | | | |
| 31 | CCCCTTGGGGAAGACGAA | N | 0.004849 | 0.002045 |
| | GGGATGCTGCAGTTCCAA | | | |
| | AAGAGAAGGACTCT | | | |
| 32 | CCTCTGCTCCTCCCTTCCC | N | 0.002769 | 0.001149 |
| | AAGGCATTGAAGCTGAAT | | | |
| | GTGCCAACTGGCA | | | |
| 33 | GCTTGTGGGTCATCTTGCA | P | 0.00569 | 3.51E−05 |
| | CCTTTACAAACAAGGAATT | | | |
| | CCCCTCTGTGCC | | | |
| 34 | CTTTTTTGTACGTAGCTGT | N | 0.009262 | 0.002535 |
| | TACATGTAGGGCAATCTGT | | | |
| | CTTTAAGTAGGG | | | |
| 35 | CCCACCATCACCTCAAACC | N | 0.003289 | 0.003276 |
| | CAATCACCCCCTCCTCTGT | | | |
| | ATGCTGTCACAC | | | |
| 36 | GGGGGCACTATAGCCACT | N | 0.008605 | 0.000682 |
| | AAACGAGGTGTGAAAGGC | | | |
| | TCAAGAGGATGACC | | | |
| 37 | AAAGTACTGCGCGACAATA | P | 0.011344 | 0.000972 |
| | TCCAGGGCATCACCAAGC | | | |
| | CGGCCATCCGGCG | | | |
| 38 | GGGGAAGCCCGGGGCCG | N | 0.031646 | 0.000409 |
| | CCCGGGACCTCGGCCCGT | | | |
| | TCCTCCGGACCCGAG | | | |
| 39 | CCGGGCTCCTAGCGGGGA | N | 0.0262 | 7.31E−05 |
| | AAAGGAAGGGGATAACTC | | | |
| | AGAGGAACAGACAC | | | |
| 40 | GAGCAAAACTGCACAAACT | N | 0.00257 | 0.00047 |
| | TGCACATTGGAAAGTGCAA | | | |
| | CAAGTTCCCGTG | | | |
| 41 | CGCCACTTCATGGAGCTG | P | 0.007682 | 0.003122 |
| | GTGACTTGTGGCCTTTCCA | | | |
| | AAAACCCATATCT | | | |
| 42 | GGTGGCTGCGCGAGGGA | N | 0.000208 | 0.000291 |
| | CCGAGTACTAGAGCTGCT | | | |
| | TGCATGCGTTACTAA | | | |
| 43 | CTGCACTGCGTGCTGGTG | P | 0.028714 | 0.013808 |
| | ACGAATCCACATTCATCTC | | | |
| | AATGGAAGGATCC | | | |
| 44 | AAGGACTCTTCCACCAGA | N | 0.008985 | 0.002854 |
| | GATGGGAAAACCACTGGG | | | |
| | GAGGACTAGGACCC | | | |
| 45 | GTGGGTCTCACCTCTCCAT | N | 0.031525 | 0.000802 |
| | TGTTCTCTTGTTCTATGGG | | | |
| | GCAGGTTTGGGG | | | |
| 46 | ACCAAGGGAGAACCAGGA | P | 0.005085 | 0.000658 |
| | AACGGAAACAGAGTGGTC | | | |
| | ATTCCCCAGCCCGG | | | |
| 47 | GGGGCATCTGGCATGGAC | N | 0.02416 | 0.00018 |
| | TGGGGTGGAAATGGGGAT | | | |
| | GTCAGTTTGAAAGC | | | |
| 48 | GCCTGAGGTGACAGACAG | P | 0.027191 | 0.001044 |
| | GGCAGGTGGTAACAAAAC | | | |
| | CGTTGAACCTCCCA | | | |
| 49 | CATGGCCAAACGTACCAA | N | 0.020811 | 0.012763 |
| | GAAAGTCGGGATCGTCGG | | | |
| | TAAATACGGGACCC | | | |
| 50 | GCTCCGTGTTGGAAAAAA | N | 0.001352 | 0.000494 |
| | GGGGTAGTGCATTTTAAAT | | | |
| | TGACCTTCATACG | | | |
| 51 | GGGGTCTGTGAGAGTACA | N | 0.002474 | 0.003327 |
| | TGTATTATATACAAGCACA | | | |
| | ACAGGGCTTGCAC | | | |
| 52 | AGTGGAGCGGCCGCCGG | P | 0.0028 | 0.003268 |
| | AGATGCCTGACGCATCTG | | | |
| | TCTGAGGAGCGGTCA | | | |
| 53 | GGCACAGGCTCTGCCGTG | P | 0.007554 | 6.88E−05 |
| | TCCTTGGAGTGAAAGACTC | | | |
| | TTTTTACCAGAGG | | | |
| 54 | CCTGTTCCCTTCATTGCTG | N | 0.047158 | 0.008599 |
| | TGAGTTGGGAGTGCATTG | | | |
| | AGAGATGATGTCC | | | |
| 55 | GACCGGAAGCAACCCCTT | N | 0.002981 | 0.004409 |
| | CACAGACACGAGCACATC | | | |
| | GGCAAACCCTATGA | | | |
| 56 | CCACTTCTGAGGAATGGA | N | 0.007523 | 0.000712 |
| | CCTGGTGTAACACACTTGA | | | |
| | ATATGTGTGATGC | | | |
| 57 | CCAGCTCTACCAGCCCCT | P | 0.004008 | 0.0025 |
| | CAAGGATCGAGAAGATGA | | | |
| | CCAGTACAGCCACC | | | |
| 58 | CGCCTGATGTCGGGACAG | P | 0.019848 | 0.009407 |
| | CCCTGCTCCCAAGTACAAA | | | |
| | TAGAGTGACCCGT | | | |
| 59 | GAGCACCTTGTTACAGTTC | P | 0.002915 | 0.000207 |
| | CGGCCTCTCAGTATGTGG | | | |
| | GCTAAATGCCAGC | | | |
| 60 | CGGGCTGGCCCACCTCGT | P | 0.005824 | 0.003599 |
| | TTTGCTAGTGAAGAGAGG | | | |
| | CGAGAAATTGCTGA | | | |
| 61 | AGTAAGGGATCGAAGACA | N | 0.015967 | 0.00069 |
| | TTTCAAATTGCTATCTCCA | | | |
| | TCTGGGCTGATCC | | | |
| 62 | GGCTGCAAGCTGGATACA | P | 0.031574 | 0.004829 |
| | TGGAATTCAGCACACTTTT | | | |
| | CTCCCTCTTACTG | | | |
| 63 | GGCATTTACGTTTCTCTGA | N | 0.006539 | 0.002363 |
| | TGCTCCCTTGAAGCCATAG | | | |
| | AATTTAGGGGCT | | | |
| 64 | GCAGCCCTAGAAAGTAAG | N | 0.020468 | 6.37E−05 |
| | CCCAGGGCTTCAGATCTA | | | |
| | AGTTAGTCCAAAAG | | | |
| 65 | TGGGAGCCCCATTGCCTC | P | 0.046562 | 0.002572 |
| | TGTCTCCTTCGGTGCCTG | | | |
| | CAGAGACTTTGTCT | | | |
| 66 | AACTGGTTGTGGGGAGGG | N | 0.024269 | 2.92E−05 |
| | AAGAGAAGGACAGGGTGT | | | |
| | TGGGGGGATGAGGA | | | |
| 67 | TACAGAACCATCCACTTGA | N | 0.003269 | 0.009058 |
| | CCTAACTACCTCCCCTGG | | | |
| | CCGCGCTCTCGCT | | | |
| 68 | TCCAAGGCCTGGATGCTA | N | 0.002557 | 0.000924 |
| | ATCAAGATGAACAGGTCG | | | |
| | ACTTTCAAGAATTC | | | |
| 69 | CCAGGGGAGGTGGGTAGA | N | 0.020742 | 4.55E−05 |
| | GCCCGAGGCCCCCCAGTA | | | |
| | GCCGACCCTGGCGT | | | |
| 70 | CCCGGGAGTGGATTCTAA | P | 0.019705 | 0.006056 |
| | ATGTGATTTTCCTAGGCTA | | | |
| | CTGCAGGAGCCCC | | | |
| 71 | GAGCACTCAACCCAGAAG | P | 0.002546 | 0.002083 |
| | GCGAAGATAGCTTTTGGTT | | | |
| | GTAGGCGGCTTCC | | | |
| 72 | GTCACCGAAAAGTGCTGC | P | 0.029826 | 0.001672 |
| | GGGATAACATCCAAGGCA | | | |
| | TCACCAAACCGGCC | | | |
| 73 | CGACTCTCAAGGCACTGT | P | 0.038275 | 0.012053 |
| | GTATGCCCTGCAAGTTGG | | | |
| | CTGTCTATGAGCAT | | | |
| 74 | GCTGTCTAGGTCCGTCCG | N | 0.006982 | 0.00795 |
| | GTGTGTCAGATTTTCCTCA | | | |
| | GATTAGATGTGCC | | | |
| 75 | GATCTCACTGACCCGTTG | N | 0.010236 | 0.007717 |
| | CCCTGTAACCACTTTCTTT | | | |
| | CCTTCTTTTGCCT | | | |
| 76 | GTTTTGGTTGTGAATCATT | N | 0.030427 | 0.000333 |
| | TGCCAGCGAGCCAAGGGA | | | |
| | GAGGCAGGGATTC | | | |
| 77 | AGGGAAGTGGGATCCGAG | N | 0.018034 | 0.000813 |
| | CCTGTAGAAGGGAGGCAT | | | |
| | GAAACTTGTGGAGG | | | |
| 78 | TTACGTTATCTACCAGAGC | N | 0.021493 | 0.008022 |
| | ACCGTGGGCTGTTACTTG | | | |
| | CCTTGAGTTGGAA | | | |
| 79 | GGGCTGAAAACTGCCCTT | P | 0.004239 | 0.002123 |
| | GGGCTGACTTTTGATAGG | | | |
| | CCATGCCTTGCCAC | | | |
| 80 | GCACAGCGTCCTGTCCAC | N | 0.004335 | 0.005296 |
| | ACCCAGCTCAGCATTTCCA | | | |
| | CACCAAGCAGCAA | | | |
| 81 | GTAACCCTCCAGTGGTGG | P | 0.019629 | 0.001216 |
| | AAGGCACACCATGGCTTC | | | |
| | CTCTGCTTGGTTTG | | | |
| 82 | GTTGAGGGAGTCAGCACA | P | 0.022339 | 0.002482 |
| | GTCCTTTCTGCAGCTTCTA | | | |
| | ACCCAGGACCATG | | | |
| 83 | GTGTCCCTGGAGCAGTGA | N | 0.014752 | 0.001847 |
| | GGGGACACCAGCAAAAAC | | | |
| | CTTCAGCTCTCAGA | | | |
| 84 | GCCTTCGGTTGTAAGTAG | N | 0.038066 | 0.03281 |
| | CCAGATCCCTCTCCAGTG | | | |
| | ACATTGGAACATGC | | | |
| 85 | CCAGACTGTGATGACTGG | N | 0.009421 | 0.006558 |
| | GAGCGGGCTGAATGAGAT | | | |
| | GGAGTGTGCATTAC | | | |
| 86 | GCCCTCTCTGTGGATCCC | N | 0.009865 | 0.015627 |
| | TACTGCTGGTTTCTGCCTT | | | |
| | CTCCATGCTGAGA | | | |
| 87 | GCCTCAGGAAAACAAGAC | P | 0.048659 | 0.001081 |
| | CTCTGTGCACCTCACTTTT | | | |
| | GGCTCACTGCAGC | | | |
| 88 | TGTAAGACGAACTTGGATC | N | 0.029902 | 0.00012 |
| | ACGGCTTGGTTCAGCAGA | | | |
| | GCATGGGGGCGGG | | | |
| 89 | AACCCAGGGCTTTAGAAG | N | 0.008492 | 8.92E−06 |
| | GCTGAGGCTGGGGGATTG | | | |
| | CTTGAAGTCAGGAG | | | |
| 90 | TCACTTGGGAGGGACGCA | N | 0.001572 | 0.000427 |
| | TAGAAGGAGCTCTAGGAA | | | |
| | CACAGTGCCAGTGC | | | |
| 91 | CAAAGGGGCATCGGAGAA | P | 0.011707 | 0.003529 |
| | GTGCAGCTGCTGTGCCTG | | | |
| | ATGTGGGAACAGCT | | | |
| 92 | ACCTGAAGCAGCAAGTGA | P | 0.039209 | 0.021103 |
| | GCGGGCTGGAGGGTGTG | | | |
| | CAGGACGACCTGTTC | | | |
| 93 | TCTCAAACCCGGTATGGT | N | 0.045389 | 0.022274 |
| | GGTCACCTTTGCTCCAGTC | | | |
| | AACGTTACAACGG | | | |
| 94 | GTCACGACATCCGAACTG | N | 0.015345 | 0.000886 |
| | GAGGGACAAGGATCTTAA | | | |
| | ACCCAAAGTACGAG | | | |
| 95 | GCTGCTAAAGTTCCAGCAA | N | 0.04698 | 0.014495 |
| | AAAAGATCACCGCCGCGA | | | |
| | GTAAAAAGGCTCC | | | |
| 96 | TAGCTGCTACCCTGGAAC | P | 0.020581 | 0.00367 |
| | GGTGGGCAGAGAGCCTAC | | | |
| | TAGGAAATGTGCAG | | | |
| 97 | GAAGAGTAACAAGAGTAG | N | 0.002215 | 0.000437 |
| | ACTGGACCAGAAATCGGA | | | |
| | GGGTGGCAAGCAGC | | | |
| 98 | CGAGAGTGCCCTTCTGAT | P | 0.00404 | 0.000435 |
| | GAATGTGGTGCTGGGGTG | | | |
| | TTTATGGCAAGTCA | | | |
| 99 | GGTGCTGATCCTACCACC | P | 0.002534 | 0.001606 |
| | TACTGCTACCTTCCTTAGC | | | |
| | TTCACCCTGGCTA | | | |
| 100 | ACCCTACTCTTCGGCCCC | P | 0.018496 | 0.001168 |
| | GCCAGCTCTCCATCTCACA | | | |
| | CTTTAAGAGCCTC | | | |
| 101 | CGTAACCAAAGAACGACA | N | 0.042699 | 0.00399 |
| | CAGAGAGATCAACAAGCA | | | |
| | AGCCACCCGAGGGG | | | |
| 102 | GAAGTAGCCCCAGTGAGT | P | 0.008798 | 0.00077 |
| | GTTAGTGATGCAGCTCTCC | | | |
| | TGGCCCCAGAGGA | | | |
| 103 | GTGAGTGGTCTCTGTCGG | N | 0.01297 | 4.58E−05 |
| | GAAAGATGTAGGGATTGG | | | |
| | TTCTCCAGGATCTT | | | |
| 104 | CACAGCCTTACTAGTTCCT | N | 0.007897 | 0.007786 |
| | TGCTTCCAGTATTTCAATT | | | |
| | GGTCTCCTCCCC | | | |
| 105 | GGCGGCCTCATCGTTCTTT | P | 0.008496 | 0.012709 |
| | GCCTTCCTGGTCACCATCT | | | |
| | GCTACGCTGGAA | | | |
| 106 | CGTGGCACCCCAAAAGGC | P | 0.037138 | 0.008695 |
| | CTCTGCTGGCATTTGCCTG | | | |
| | TGATGACAAAGAC | | | |
| 107 | GGCTTTTCAATTCTGTGGA | N | 0.042964 | 0.0271 |
| | CTTTGTACCATTTGGCTTC | | | |
| | ACCTTGTACTGC | | | |
| 108 | AAAGCAGATATTTCCCGGA | N | 0.000457 | 0.001746 |
| | CCCAGCGCGGCCTCAACC | | | |
| | AGGGCAGGAAAGA | | | |
| 109 | ACGGCGTGGAGGACTTTT | P | 0.012031 | 0.001874 |
| | CCGTGAGCCAGACGATGC | | | |
| | TGGAGGAGGTATTC | | | |
| 110 | GGGGCTACATTTGTTCATT | N | 0.030895 | 0.024754 |
| | TCCAGCAGTAGCATAAACT | | | |
| | TACGGTGACATG | | | |
| 111 | GTCCTCACGTTCCCAGGA | P | 0.016356 | 0.006994 |
| | GGGCGGCTTCACCCTTCG | | | |
| | TAACCAGGAGACAA | | | |
| 112 | CTGAACTGCTAATGTGGCT | N | 0.031398 | 0.004786 |
| | GCTTTGTAGGGAATGGAC | | | |
| | TAATATCAGTGTG | | | |
| 113 | TGCCAAGATACATTGACAC | P | 0.021675 | 0.012145 |
| | TGAACATGGAGGCAGCCA | | | |
| | GGCCCGTTTCCTC | | | |
| 114 | CTTGCGGAAAATGAGAATT | P | 0.034572 | 0.015218 |
| | GATGGTGTCCCCAATGCC | | | |
| | CCACCTCACAGAG | | | |
| 115 | TGCATGGGGGTACCCCAA | N | 0.001618 | 0.000135 |
| | TCTGAAGTCAGTAAATGAA | | | |
| | CTAATCTACAAGC | | | |
| 116 | AACAGCATCCTCTTCCACG | P | 0.038694 | 0.001479 |
| | CTCAGAAGTGTTCTGGTTG | | | |
| | GGGCCAGGCATG | | | |
| 117 | CAGATCTACTGGCGAGCG | N | 0.025043 | 0.00406 |
| | ATGAAAATGTTGCAGGGA | | | |
| | GAGTCAGCAGAGGC | | | |
| 118 | CCGTGGGCTGTGCCAAGT | P | 0.017698 | 0.011077 |
| | GTGCCCAGGGATGTGTTT | | | |
| | GCAAAGGGACATCT | | | |
| 119 | GGGATTCTGTGACTGGAA | N | 0.021955 | 0.002057 |
| | AAGGTGACAAGTTGGTGA | | | |
| | CTTTGACACTGCAG | | | |
| 120 | TCCTGGCCATGAGGACAA | N | 0.009159 | 0.003566 |
| | AAATTACTGAGTGGCCCTT | | | |
| | AAAGAGGGAAGTT | | | |
| 121 | TAATCCCGTTATGGACTCT | P | 0.025232 | 0.044454 |
| | GTCTCCAGGAGAGGGGTC | | | |
| | TATCCACCCCTGC | | | |
| 122 | TGTTGGCAGCGACACCAT | P | 0.019066 | 0.001334 |
| | CCCATACAGGCTCTTACCT | | | |
| | CTTCTCCTGAGGG | | | |
| 123 | GGCAGTTGTCTGCATTAAC | N | 0.010001 | 0.000112 |
| | CTGTTCATACACCCATTTT | | | |
| | GTCCCTTTATTG | | | |
| 124 | AGCTTTCTGCACCCCCAGT | P | 0.040871 | 0.002595 |
| | GGCATCTCCTCATCACGTT | | | |
| | CTGTGCCGTCCT | | | |
| 125 | GGGGCTTTCGTGTCCCCC | N | 0.014516 | 0.000335 |
| | TGTGCGGTCAGTGTTTTCA | | | |
| | GTACCACCTCTCT | | | |
| 126 | GAGCAGGTGAAGCCATCA | N | 0.011877 | 0.003317 |
| | AAGAATGGGGTGACCACG | | | |
| | TGACCAACTTGTGC | | | |
| 127 | AGCCCTGATGATTGGCCC | N | 0.044672 | 0.003063 |
| | CACCTCCTGCTGCCCCAT | | | |
| | AACCCTCTCTTCAT | | | |
| 128 | AAAGAAAGCTGGGCCTGT | N | 0.026121 | 0.026357 |
| | CGAAGGATGACAGGGATG | | | |
| | TGCTGCCAGGTTGC | | | |
| 129 | TCACCCGCACTGAGTCAA | N | 0.032827 | 8.36E−06 |
| | CAGACTGAGCGCGTCCAG | | | |
| | GCCTGACAGCTCTG | | | |
| 130 | GGTGTGACTTGCCTTATTG | N | 0.027346 | 0.008191 |
| | AACTGATACTGGCATATCT | | | |
| | GACTGTAAGCAG | | | |
| 131 | CAAGCCTCACTTTTCTGTG | N | 0.008937 | 5.37E−06 |
| | CCTTCCTGAGGGGGTTGG | | | |
| | GCCGGGGAGGAAA | | | |
| 132 | TGAGGACAGTTCAGAGGA | P | 0.013436 | 0.004082 |
| | GATTCAGACGAGTGTCG | | | |
| | CTGCGTGAGTGGCC | | | |
| 133 | GCTCTCTCCCATCCAAGTG | N | 0.004927 | 0.003 |
| | ACCAGATGCCCTACTCAG | | | |
| | CTTCCATCACCCC | | | |
| 134 | CAGCTGGTTTCCTGGGTAT | P | 0.008471 | 0.002079 |
| | GCCTGGACTGTTGCCCAG | | | |
| | TGTAAGATCTGTG | | | |
| 135 | AGGAGGTACAGACGGTGG | P | 0.018423 | 0.013478 |
| | AGGATGGGGTGTTTGACA | | | |
| | TCCACTTGTAATAG | | | |
| 136 | GGGGAACACACCTGAAAC | N | 0.030934 | 0.001904 |
| | TAGAGGAACAGCTTATGTG | | | |
| | GTCTATGAGGACA | | | |
| 137 | CGGACAGTGATGGCTCTT | N | 0.000473 | 0.000866 |
| | GGAAATGGGTGGATGGCA | | | |
| | CAGACTATAGGCAC | | | |
| 138 | ACCGTGGTGAACCCTTGG | P | 0.040643 | 0.005937 |
| | GGGGAGGTTCTAGCCAAA | | | |
| | GCTGGCACAGAAGA | | | |
| 139 | GGGAGTGGTGGAGCCAGT | P | 0.025216 | 0.01514 |
| | CGCTGTAACACTGAGCCT | | | |
| | CAGAGACGAACCAA | | | |
| 140 | AGGCCTTAAGCTTTGGAC | N | 0.004216 | 0.004512 |
| | CCAAGGGAAAACTGCATG | | | |
| | GAGACGCATTTCGG | | | |
| 141 | ACTGCTGCGTCATTACAG | P | 0.002145 | 0.000493 |
| | GGCACAGGCCATGGATGG | | | |
| | AAAACGCTCTCTGC | | | |
| 142 | TCTGGAAGGGGACAGTGA | N | 0.04558 | 2.54E−05 |
| | AAAGAGGAGTGACAGGAG | | | |
| | GGAAAGGGGGAGAC | | | |
| 143 | CCTGGTCAAGTGCTGGCT | N | 0.017756 | 0.012466 |
| | CTGCTGTCCTTGCCTTCCA | | | |
| | TTTCCCCTCTGCA | | | |
| 144 | TAGGTGTGGTGGCGTTAT | P | 0.029749 | 0.004606 |
| | GGCAGCCCGGCTGCTGCT | | | |
| | TGGATGCGAGCTTG | | | |
| 145 | CCTGCAGTGTAAGTACAG | N | 0.005883 | 0.00061 |
| | CACACTGTCAAATTCTTTT | | | |
| | CCTTAAGGTGCAC | | | |
| 146 | GGGCTTTTCCAAAAGCAAA | N | 0.014468 | 0.001402 |
| | CAAAGATAGGTTCCTCAG | | | |
| | GTGACCAAAACTG | | | |
| 147 | ACATCTTTCTGGCACATAA | N | 0.007055 | 0.001236 |
| | CTGTCTCCTTAACCACTGG | | | |
| | AACAGTTCAGCC | | | |
| 148 | GGCATTAGAGATCCAGCA | N | 0.013353 | 0.00265 |
| | CATTCTCAGTACTGTGGTG | | | |
| | CAGTATTAGCCCA | | | |
| 149 | CGCCCGAATCTGGCTCGG | N | 0.013846 | 0.002135 |
| | CGGAATACCTCTTAGACAA | | | |
| | GCACACCCTGGGG | | | |
| 150 | GAACTTGGAGAGCATCAG | N | 0.048169 | 0.008186 |
| | GAAGGCCCAGCTGAAATC | | | |
| | AGAGAATCTGCTCG | | | |
| 151 | TCCTCCTGAGCCTACTGC | P | 0.023875 | 0.004032 |
| | CAAACGTCCTCAGTGTTGT | | | |
| | CTGCACCTGCTCC | | | |
| 152 | CAGCTTCCAGTGGTGGCC | N | 0.011484 | 0.003845 |
| | GTAGACTTGGCTCGGAAC | | | |
| | TTAGTGGCACCAGA | | | |
| 153 | TCCCATGTTTTTACCCTGC | N | 0.000264 | 0.000431 |
| | CCCTGCCTTGATTAGACTC | | | |
| | CTAGCACCTGGC | | | |
| 154 | GTTATGCTTGTATTGAATG | N | 0.024561 | 0.005564 |
| | CTGTCTTGACATCTCTTGC | | | |
| | CTTGTCCTCCGG | | | |
| 155 | CCCGGCCAACATCAAGTG | N | 0.013639 | 0.003569 |
| | ACTTTATAGCTGCAAGAAA | | | |
| | TGTGGTATGTGGA | | | |
| 156 | CTGCTGCGACTGATGCCA | P | 0.010739 | 0.005759 |
| | GGACAACCTTTCTCCCAGA | | | |
| | TGTAAACAGAGAG | | | |
| 157 | CCTGCATAACAACACTGG | N | 0.006612 | 0.000644 |
| | GCCTTCTTAACTAAAATGC | | | |
| | TCACCACTTAGCC | | | |
| 158 | GATGGACTGTGCCCGGGT | N | 0.02211 | 0.000181 |
| | TCTGGTCATGGACAAGGG | | | |
| | GCAGGTGGCAGAGA | | | |
| 159 | GCTGTGCAAAGGTTGAGA | N | 0.01233 | 0.008166 |
| | GCTATTGCTGATTAGTTAC | | | |
| | CACAGTTCTGATG | | | |
| 160 | GTTGGTGGTGTTTGAGGG | N | 0.000869 | 0.000484 |
| | TTGGCTAGAAATGAAAGCC | | | |
| | TGGATTTTGTGCC | | | |
| 161 | CAGGCACCTGGCTGAGTG | P | 0.011278 | 0.000605 |
| | TGCTGGAGTGAGGATCTT | | | |
| | GAACAGAAACTTCC | | | |
| 162 | CAGTTATGGAGGACTTGTA | N | 0.022569 | 0.000211 |
| | TGGAGAAATTTAAGTCTTC | | | |
| | ACTGAGGGCCAC | | | |
| 163 | TCCCCACTATAACAGTTGC | N | 0.034684 | 0.009182 |
| | TGCCGCCGGAAGTACAGA | | | |
| | CCAGAAGCCCCTG | | | |
| 164 | TGTCCTGGCTTCCCCTCC | P | 0.041505 | 0.013571 |
| | CAAGGAGGATGAGGATGG | | | |
| | TGCCTCTGAGGAAA | | | |
| 165 | CCACCTCGAGAACCAAGG | N | 0.013801 | 0.002948 |
| | ATACTTTCGGAAGAGGAG | | | |
| | CAGGAAATGTTCAG | | | |
| 166 | CTTTGTTCCTGGGGAATTC | P | 0.012359 | 0.000189 |
| | ACTTCTCTTCCTCCCTCAT | | | |
| | GGAAGATGCAAG | | | |
| 167 | GCCATATTGGAGTAGCGA | N | 0.031803 | 0.001028 |
| | GGAATCTGATTCCAAGCAA | | | |
| | AAACCAGACAATG | | | |
| 168 | GCTTCAGGCGGTAAACCA | N | 0.012877 | 0.004994 |
| | ACAGCTCACAAAGGAGAA | | | |
| | AGAGCACTACCAGG | | | |
| 169 | GAAACTGTTGAAGCTGCA | N | 0.021787 | 0.010585 |
| | GAACCAACGAGGTGGCCG | | | |
| | AATCCTTCTTCAGG | | | |
| 170 | GAGGGAGAAGAATAAAGC | N | 0.003394 | 0.000978 |
| | AGCTGCCTGGAGCCTATT | | | |
| | CACTATGTTTATTG | | | |
| 171 | CAGGTCCTGCAGTCTGGC | P | 0.042554 | 0.024263 |
| | TGAGCCCTGCTTGGTTGT | | | |
| | CTCCACACACAGCT | | | |
| 172 | TAAGGCCCTGCACTGAAA | P | 0.02409 | 0.004075 |
| | ATGCAAGCTCAGGCGCCG | | | |
| | GTGGTCGTTGTGAC | | | |
| 173 | GGTGTCCATCAGTAACTAC | P | 0.032171 | 0.038592 |
| | CCCCTTTCTGCTGCCCTCA | | | |
| | CCTGTGCAAAAC | | | |
| 174 | GATCCAGCCATTACTAACC | N | 0.005094 | 5.35E−05 |
| | TATTCCTTTTTTGGGGAAA | | | |
| | TCTGAGCCTAGC | | | |
| 175 | GGGCGGCATTTACACTGT | N | 0.017674 | 0.00014 |
| | GCAAGTATTGAGAAGAGT | | | |
| | GCATAAAGACAGGG | | | |
| 176 | CATCTCTGTGGCAGCGGC | P | 0.015266 | 0.009136 |
| | AGCTATTTACATGGCCTCA | | | |
| | CAGGCATCAGCTG | | | |
| 177 | CGTGTGCCACTTGCCCAG | P | 0.042064 | 0.009795 |
| | CTTCTTGGGCACACAGAG | | | |
| | TTCTTCAATCCAAG | | | |
| 178 | TCTCCCAAATAAGATGTGC | N | 0.015764 | 4.09E−06 |
| | TGCTTACCGAGGTATCAC | | | |
| | GGGGTGGGGCTCC | | | |
| 179 | AGGGACTTTGTTTAGGCCA | N | 0.003346 | 0.00068 |
| | AGGAAGGAGCGGAAGTAG | | | |
| | GGCAACTCGGTCC | | | |
| 180 | CCTGCTAAGTCCGCTCCT | P | 0.023975 | 0.000353 |
| | GCTCCAAAAAAGGGCTCC | | | |
| | AAAAAGGCGGTGAC | | | |
| 181 | GTGGTCTGTAGCCCAATAA | N | 0.005178 | 5.12E−05 |
| | CTGGGGAACGAGTTACAG | | | |
| | ACAAACATCACCG | | | |
| 182 | CAGCTCATGCCCTCAATGT | N | 0.047967 | 1.34E−05 |
| | TTATATTGTGTTATCTGTTG | | | |
| | GGTCTGGGACA | | | |
| 183 | TTGGGAGCTGAAGAATACT | N | 0.012001 | 2.04E−06 |
| | GGACGGGGCTTCGGAGAG | | | |
| | GAAGGATGGTCCA | | | |
| 184 | ATTGCTCCCCAGACTGAAC | P | 0.011266 | 0.002311 |
| | AGAAACCTGGCCGCCGGA | | | |
| | TGGGACCTCCTTT | | | |
| 185 | CCAAGGTGTTAAGGGGAT | N | 0.000574 | 0.00161 |
| | AGTACCTCCCAATTCAAGC | | | |
| | AGAGAAACTGACC | | | |
| 186 | GGGGCACATGTTGTAAGA | N | 0.045998 | 0.00018 |
| | AACTGATTGGAAGGGGAA | | | |
| | ATGTGCAGCTCTCC | | | |
| 187 | ACTGCTGGCAGCGGCTTT | N | 0.041264 | 0.00259 |
| | CTGTATTCTGCCACACCAG | | | |
| | GGGCAGATGTTTG | | | |
| 188 | CACCCCAAGCAGTACGCT | N | 0.00743 | 0.001722 |
| | TGCTGGTCTAAGTCTTAAC | | | |
| | CCCAGGACTCAGA | | | |
| 189 | CTTTTCCAAGTTCCCAAGG | P | 0.041075 | 0.015325 |
| | CCTACAGCTGAAGCCCTTA | | | |
| | GGTACCTGTGTT | | | |
| 190 | CATACAGTAATCATGCTGC | N | 0.017807 | 0.004063 |
| | AGAAATTTGCAGTCTGCAC | | | |
| | CTTATGGATCAC | | | |
| 191 | CCTCTTGAGCTGGAACGC | P | 0.003337 | 0.004296 |
| | CTGAAACTGGAGCCTCAC | | | |
| | GAAGGGCTGCTGCT | | | |
| 192 | AGTGATTGCCTGGGCCAA | N | 0.035584 | 0.000196 |
| | GTGGCAGGTTGGGGAGGA | | | |
| | TGGCTGCAAAGAAG | | | |
| 193 | CTTCCAGTCTTTTTAGAAC | N | 0.006781 | 0.000304 |
| | GTGGTGGAGGAGGGTTGT | | | |
| | GTGTGCCCCAGGG | | | |
| 194 | GGGGGGTAGAATTTAGTA | N | 0.010088 | 0.003144 |
| | AATATTCCAGCCGGTCGTT | | | |
| | TTATGCACAAGGC | | | |
| 195 | TGATCCGAAGGAGGAGTG | P | 0.034854 | 0.004035 |
| | GCGCTGGGCGCTGGACTC | | | |
| | GCTGGTGTGAAAAT | | | |
| 196 | GAGTCTCAAGTCCGTATGT | N | 0.000755 | 0.002992 |
| | AAATCAGATCTCCCCTCTC | | | |
| | ACCCCTCCCACC | | | |
| 197 | CAGATTTGGCACCTACTCC | P | 0.004675 | 0.001488 |
| | TGCCCCACAGAGCACACC | | | |
| | ACGAAACACTGTC | | | |
| 198 | GAGCTTCCCGAGAATGGG | N | 0.001177 | 0.000287 |
| | GCCTGGGTTTGATTCATCT | | | |
| | GTTTTCTACAGGG | | | |
| 199 | GAGACCAGTAGATTTTCAA | N | 0.041733 | 0.000244 |
| | TGGGAAATGTACCTAGCAA | | | |
| | GCTGGTTCTTGC | | | |
| 200 | AACCAGGGGCCATGAATC | N | 0.034866 | 0.000449 |
| | ACCTTTTGGTCTGGAGGG | | | |
| | AAGCCTTGGGGCTG | | | |
| 201 | GTCCCTGTCCCTCCCAAA | P | 0.028729 | 0.003586 |
| | GCACAGAGCACAGAAATG | | | |
| | AGGCCGTTTACATG | | | |
| 202 | GCAGATAGAGTGTTACCG | P | 0.005896 | 0.000645 |
| | ACGGGTGGAAAAGCTACG | | | |
| | GAATCGCCAGGATG | | | |
| 203 | CAGCCATAGGTGCAGTTT | N | 0.007314 | 0.006682 |
| | GCTTCTACATGATGCTAAA | | | |
| | GGCTGCGAATGGG | | | |
| 204 | CCCTGGTATTGATTTCTCA | P | 0.007792 | 0.003889 |
| | GGACTTTGGAGGGCTCTG | | | |
| | ACACCATGCTCAC | | | |
| 205 | GGCTGGCAGTCTTTGTCG | N | 0.034813 | 0.000624 |
| | TTGTTCATTCTGGGGATAA | | | |
| | AGGGGAACTAGGC | | | |
| 206 | CCAGAGCCTGTGATGCCT | P | 0.007285 | 0.001073 |
| | CCTCAGCAGGTAGAGCAG | | | |
| | ATGGAAATACCACC | | | |
| 207 | GGGACTCAGCATTTTCCA | N | 0.021834 | 0.000496 |
| | GTCTTTTTCAGGGGTAGAC | | | |
| | AGGGGAGCCTGGG | | | |
| 208 | CCTGTGTTTGCATCCTCTG | N | 0.005121 | 0.008657 |
| | TTCCTATTCTGCCCTTGCT | | | |
| | CTGTGTCATCTC | | | |
| 209 | GGGTGCCTTCCTTGGTCA | N | 0.004562 | 0.002999 |
| | CCAAGGCAGTGCGTGCAC | | | |
| | GTTAGGGTTTCCTT | | | |
| 210 | CTCAAGGTCATGCAGTTAG | N | 0.02031 | 0.005178 |
| | TAAGTGGCAGAACAGGGA | | | |
| | CTTGAACCAAGCC | | | |
| 211 | CCTCCACGTGATTCCTACA | P | 0.003558 | 0.001324 |
| | GCAGTTCAAGCCGCGGAG | | | |
| | CACCAAGAGGTGG | | | |
| 212 | TGCTGCTCCTGCTGCCCC | P | 0.013625 | 0.010856 |
| | ATGAGCTGTGCCAAGTGT | | | |
| | GCCCAGGGCTGCAT | | | |
| 213 | CTATTAACGCTACGATGCC | P | 0.015462 | 0.004802 |
| | TGAACCTACCAAGTCTGCT | | | |
| | CCTGCCCCAAAG | | | |
| 214 | GATGGTTCTGATGCTGTCA | N | 0.017559 | 9.16E−06 |
| | GCCTCTGGGTGCAAATTCT | | | |
| | GAGGGCCCGGGA | | | |
| 215 | CCCTCACGCACCCGCTCA | N | 0.009263 | 0.002613 |
| | CGCACCCTCGGTGAATCC | | | |
| | TTGGTGATGATTTT | | | |
| 216 | AGTACCACTCCAAAGGCA | N | 0.007405 | 0.001662 |
| | AGGAACCATGATTGACAAC | | | |
| | AGTCAAGCTGTGG | | | |
| 217 | TGGAAGCCCTCACCAAGC | P | 0.027769 | 0.002064 |
| | ACTTCCAGGACTGACCAG | | | |
| | AGGCCGCGCGTCCA | | | |
| 218 | CCCCTACTTATTGCCACAG | N | 0.023489 | 8.38E−06 |
| | AGGAGGGATCTTTTCCATA | | | |
| | ACTGAAGGGGAG | | | |
| 219 | GCACGACGATGAGGTGAC | N | 0.004535 | 0.000151 |
| | AGTCACGGCCCTGGCCAA | | | |
| | CGTCAACATTGGGA | | | |
| 220 | GAATACTTCTCTTGCTGAG | P | 0.017275 | 0.009131 |
| | AGCCGATGCCCGTCCCCG | | | |
| | GGCCAGCAGGGAT | | | |
| 221 | GCACAGTTGAGGAGCCAG | N | 0.014228 | 0.002628 |
| | AGACTTCTTAAATCATCCT | | | |
| | TAGAACCGTGACC | | | |
| 222 | CTTTAATTCTTGGGCCTCC | P | 0.00127 | 0.001745 |
| | AATAAGTGTCCCATAGGTG | | | |
| | TCTGGCCAGGCC | | | |
| 223 | TACCTGGCTACAGAAAGAA | N | 0.011287 | 0.003864 |
| | GATGCCAGATGACACTTAA | | | |
| | GACCTACTTGTG | | | |
| 224 | GACCCCCTTTTAAGCCAGT | N | 0.041676 | 0.020247 |
| | GAGCTGGGCTTCAGTTTTT | | | |
| | CCCAGGCCATGC | | | |
| 225 | CCCCTGCAAGGGTAGAGT | P | 0.033071 | 0.000577 |
| | CAGGTGAGAGTCCCTTGG | | | |
| | TGAGTCATTTGTAC | | | |
| 226 | GCCCAGTACTGGAGAAAA | N | 0.014944 | 5.33E−05 |
| | TGAAACTGGGATTGACCC | | | |
| | ATCAAGATGCTTGG | | | |
| 227 | GGGACGAGACAGGTGCTA | N | 0.005603 | 0.00053 |
| | AAGTTGAACGAGCTGATG | | | |
| | GATATGAACCACCA | | | |
| 228 | CCCCAGTGTGTATAAGCT | N | 0.012426 | 0.006972 |
| | GGCATTTCGCCAGCTTGTA | | | |
| | CGTAGCTTGCCAC | | | |
| 229 | CTGTCCCGCTGCGTGTTTT | P | 0.003192 | 0.005766 |
| | CCTCTTGATCGGGAACTC | | | |
| | CTGCTTCTCCTTG | | | |
| 230 | GACTCTGATGTTGGGTAG | P | 0.009787 | 0.002361 |
| | CTGGCCTCTGTGGGGATT | | | |
| | GTAAGTGCCCTGAG | | | |
| 231 | GAGGTGTTTGCATGTGGC | P | 0.01286 | 0.002536 |
| | CATTACCGTCATTGGCCTG | | | |
| | TGAAGCATTGGAC | | | |
| 232 | CACACTGGGGCTGCCTTT | N | 0.001533 | 0.000314 |
| | CTCTGACTCTGTCTTCCCC | | | |
| | AAGTCAGGGGGCT | | | |
| 233 | TACCACTGCAAAGTGATG | N | 0.002811 | 0.001328 |
| | GAAAAGGGTGGAGAACAG | | | |
| | GGGAGTAGCCAGGC | | | |
| 234 | GCTTTCTTAGGGAAATGAC | N | 0.01812 | 0.001642 |
| | AGGGCAAAGCAATTTTTCT | | | |
| | GTTGGCTTTGGG | | | |
| 235 | CTACGCCATGGGACATCT | N | 0.008083 | 0.01079 |
| | AATTCAGAGGAAGAAGGT | | | |
| | CCATGTCTTTGGGG | | | |
| 236 | GAGGATCATTACAGAGAC | N | 0.013942 | 0.000257 |
| | AGACTCTCCCGAGACATG | | | |
| | GGCCACACTGATAG | | | |
| 237 | CTGTGGGAGGGCTTCTTC | N | 0.028462 | 0.000297 |
| | CCTGTGCGCTGTTGCCCA | | | |
| | TCCAAGCCTAATAT | | | |
| 238 | GTAACTGTAAGTTCACATC | N | 0.008932 | 0.000714 |
| | AACCTCATGGGTTTGGCTT | | | |
| | GAGGCTGGTAGC | | | |
| 239 | CCTGGCCAAGTGAGGAAG | N | 0.024086 | 0.000229 |
| | GAAAGCAGAAAGGTGACG | | | |
| | ATTCTCACTCACCT | | | |
| 240 | CAGCCTGACGAGCTGCCC | N | 0.010261 | 0.015509 |
| | GAGGTGGATAACCTGACA | | | |
| | CTGGACGGGAATCC | | | |
| 241 | GCTCTGCACCATCCCTCA | P | 0.041028 | 0.01213 |
| | CCCAGACCGTAGACACCA | | | |
| | GGGAACCACATCTA | | | |
| 242 | CTGCGAGTTTTCGGGTGG | P | 0.027036 | 0.011983 |
| | GCAGACGCACTGTTGAAT | | | |
| | CTGGTAGCCAGGGT | | | |
| 243 | GAGCGTGATGATTGGGTG | P | 0.000645 | 0.001456 |
| | TTCATACGCTTGTGTGAGA | | | |
| | TGTGCCACCCTTG | | | |
| 244 | GAAACATTCTAGTAGCCTG | P | 0.001124 | 0.000461 |
| | GAGAAGTTGACCTACCTGT | | | |
| | GGAGATGCCTGC | | | |
| 245 | AGGTCTCCTCTGGGAGGT | P | 0.031257 | 0.004239 |
| | CTTGGCCGACTCAGGGAC | | | |
| | CTAAGCCACGTTAA | | | |
| 246 | CAGGCTCATAGCAGCTAC | N | 0.008003 | 0.004521 |
| | TGTGTAGAAAATTCCCCCT | | | |
| | ACTTCTAATTTGC | | | |
| 247 | TCTTGCTGACAGAATAGGT | N | 0.009211 | 0.003011 |
| | TCCGTTCTGGGCGGTGGT | | | |
| | TCTCGAGCCTGCC | | | |
| 248 | CCTCAGCAGCTGGTAATCT | N | 0.006996 | 0.013739 |
| | TGCTCTGCTTGACAACATC | | | |
| | TGAGTGCAGCCG | | | |
| 249 | CATTCTGGGACTACCGTG | N | 0.003676 | 0.001577 |
| | AAGCCTGGAGTAGGGAGA | | | |
| | GCGAGTTTGGGAGC | | | |
| 250 | GCTGGCGTGCCCATGTTG | N | 0.001542 | 0.002048 |
| | CAGATATTTTCCCGAGTTC | | | |
| | CCCAGAATGGATG | | | |
| 251 | GACCTCCAGAGTGAAGAT | N | 0.001336 | 8.77E−06 |
| | GGGTGACTAGATGATATGT | | | |
| | GTGGGTGGGGCCG | | | |
| 252 | GGGAACTGGCATTACTGG | N | 0.024337 | 0.00072 |
| | AACTAATGGTTTTAACCTC | | | |
| | CTTAACCACCAGC | | | |
| 253 | CTGGGCAGTGAAGTGGAT | N | 0.026178 | 0.007556 |
| | ATCACTGAAGGAGATAGG | | | |
| | AAGCCAGACTACAC | | | |
| 254 | GCAAAAGTGAAGCAGGAA | P | 0.012252 | 0.001468 |
| | AGAAGGGCCCTGGCAGGC | | | |
| | CAACAGGCTCAAAG | | | |
| 255 | AACAGCTCTGTGTGTGAA | N | 0.000599 | 0.00067 |
| | GGTGAGGACTCTTGGAAG | | | |
| | CAGGCCATCCTGGC | | | |
| 256 | CCATCTCCGGGACGTTCT | P | 0.046366 | 0.032238 |
| | CGGCTCTGCCTCATTGTGT | | | |
| | GCAGAAACTGTGG | | | |
| 257 | GGAGGCCAGACGTTGACG | N | 0.03009 | 0.000127 |
| | CTGCAGGGAGAGGGTGGT | | | |
| | GGGCGCAGCCGCTA | | | |
| 258 | GGTGGGGATTCTGGAACA | N | 0.01535 | 0.000204 |
| | ATCATCTTAGGGGGTGTG | | | |
| | CCATGCTGTTCCTG | | | |
| 259 | AGCTTCCCAAGCTGTATGT | N | 0.014478 | 0.009585 |
| | GAAGCTACCTTACTGTGTG | | | |
| | AGTTGTGCAATT | | | |
| 260 | ACTGCTGCTTCCTACCTGC | N | 0.003324 | 0.00088 |
| | AAGACGAACAATGTATGTT | | | |
| | TCAAGGGTGAGC | | | |
| 261 | GCGCCTCCAGGCCAAGAA | N | 0.024024 | 0.002229 |
| | GGAGGAGTTCATCAAGAC | | | |
| | TTTATCCAAGGAGG | | | |
| 262 | GTGGAAAGGATGGGGTGG | N | 0.006424 | 6.9E−05 |
| | AATACAGTTGTGGGCTATT | | | |
| | GGTAAGGTCCCAG | | | |
| 263 | TATTGCAGCCATCCATCTT | N | 0.001884 | 0.000123 |
| | GGGGGCTCATCCATCACA | | | |
| | CCCGGGTTGCTAG | | | |
| 264 | CGGCCCCTGAGCAAGACA | N | 0.002721 | 0.004042 |
| | GTACGCTTCAACGTGCTCA | | | |
| | AGGTCACCAAGGC | | | |
| 265 | CCCAGCTGAACCCGAGGC | N | 0.019929 | 0.004179 |
| | TAAAGAAGATGAGGCAAG | | | |
| | AGAAAATGTACCCC | | | |
| 266 | TAGCAGCTTGGGCACCTC | P | 0.01371 | 0.001928 |
| | CACTCTGTGCGGTCTGAT | | | |
| | GGCCCCAGCAAGGT | | | |
| 267 | GACCGCTATGCTCAGGAC | P | 0.037006 | 0.005895 |
| | ATGGGAGACAACTGCATTA | | | |
| | CTCAGTGATCAAG | | | |
| 268 | TGGAGGTGGTTTTGGTGG | N | 0.045541 | 0.006933 |
| | GAATGACAACTTTGGTCAT | | | |
| | GGAGGAAACTTCA | | | |
| 269 | ATGTGGACTGCCCTACATT | P | 0.020335 | 0.002738 |
| | TGGCCTGTGCCACTGGCC | | | |
| | AACCGGAAATGGT | | | |
| 270 | TTTTGTTAACGTCTGCCAC | N | 0.016459 | 0.002366 |
| | CCCCACTCTCACCCCCAA | | | |
| | GCTCTAAGCCCCC | | | |
| 271 | ACATGTTCCGATGCCTGTG | P | 0.003918 | 0.002067 |
| | GAAGACATGCCGACGTCT | | | |
| | CCTCTGCCTAGGG | | | |
| 272 | GAGTCTTCGTGGATGATGT | P | 0.002408 | 0.000801 |
| | GACCATTGAGGACCTGTC | | | |
| | AGGCTACATGGAG | | | |
| 273 | ACTCTGGCCCCTATGGCG | P | 0.005261 | 0.000485 |
| | GTGGAGGCCAGTACTTTG | | | |
| | CAAAACCACGAAAC | | | |
| 274 | TCTTCCATACATTAGTTCC | P | 0.018924 | 0.008322 |
| | CACCATCGCATGCCCAGG | | | |
| | GACCACTGCCTGG | | | |
| 275 | AAGCTAAGGCCGCGTTGG | N | 0.007037 | 0.00584 |
| | GGTAAGGCCCTCACTTCAT | | | |
| | CCTGCGACTAGCA | | | |
| 276 | CGACCGGCTCGTATTCCG | N | 0.032215 | 0.013902 |
| | ATCAGTCGCTTCCATTGTT | | | |
| | AGCATCGTACACG | | | |
| 277 | AGATGTGTTTTCAGAGCTA | N | 0.002118 | 0.001912 |
| | GGTACAGAGGAATGTTTG | | | |
| | CTACCTTTAGCGG | | | |
| 278 | CGGGTGCAAGCCCGTGTG | P | 0.006615 | 0.002452 |
| | TCTGGCCTCTTTCCTCGTG | | | |
| | AAGACGATGTGTC | | | |
| 279 | CAGTGGCTACCACCTGTA | P | 0.017095 | 0.013578 |
| | ATCTCAGCAGTTTGGGAG | | | |
| | ACCAAAGCAGGACG | | | |
| 280 | CAAGCAAAATTGTGGGCA | N | 0.025783 | 0.003476 |
| | AGAGAATCCGCGTGAAAC | | | |
| | TAGATGGCAGCCGG | | | |
| 281 | GAGGGCACCAGGCACAAC | N | 0.02207 | 0.000134 |
| | GACATCGAGCTCTACAGC | | | |
| | CAGTACCTGGAGGG | | | |
| 282 | TCTGCAAAGGGGCGTGCA | P | 0.00797 | 0.026713 |
| | GCTGCTGTGTCTGATGTG | | | |
| | GGGACAGCTCTTCT | | | |
| 283 | GTGTAAGGGTCCAGCTGA | N | 0.021321 | 0.001836 |
| | TCAAGAATGGCAAGAAAAT | | | |
| | CACAGCCTTTGTA | | | |
| 284 | GATGGGCACCTGGATAAC | N | 0.016792 | 4.55E−07 |
| | TCAGGATGGGGGCTGCTC | | | |
| | ACAAAGACCACATC | | | |
| 285 | GGAGCCCCTTGGAGTATG | P | 0.038278 | 0.009229 |
| | GCTTTTCACATGGGCTTCT | | | |
| | ATACCGCTTCGAC | | | |
| 286 | ACTGTCAGGCCAGTGCTG | P | 0.020498 | 0.003103 |
| | CTGCGGATGTGAGAAACC | | | |
| | GGTGATCCGAAGGC | | | |
| 287 | CCCCTGGGCTATCATCTG | P | 0.007544 | 0.000284 |
| | CATGGGGCTGGGGTCCTC | | | |
| | CTGTGCTATTTGTA | | | |
| 288 | CACAAGAGTGGTCATAAG | N | 0.014948 | 0.00028 |
| | GGGGTTTGAACTGAGTCC | | | |
| | CACTACCTCGGGGG | | | |
| 289 | ACCCCTGGGCTACCATCT | P | 0.007981 | 0.001869 |
| | GCATGGGGCTGGGGTCCT | | | |
| | CCTGTGCTATTTGT | | | |
| 290 | CCCTCTCAAGTAATGGCTC | N | 0.010546 | 0.000671 |
| | AGCTAATAAAGGCGCACAT | | | |
| | GACTCCCAAAAA | | | |
| 291 | GTACTTCGGGGCTCTACA | N | 0.009447 | 0.000317 |
| | GACAATCTGATGGATGACA | | | |
| | TAGAAAGGGCAGT | | | |
| 292 | TTCTCAGGAATCGGCGGG | N | 0.037168 | 0.000823 |
| | AAGAAGCCCCCTTGATGG | | | |
| | AGTCTGGTGGGGTT | | | |
| 293 | TGGTATTTGGGCAGCTGG | P | 0.002393 | 0.002545 |
| | TGATCGTTGGTCCCGGCG | | | |
| | CCCTTTCTTTACTG | | | |
| 294 | CTGTATGCCCAGGGAAAG | N | 0.003513 | 0.000732 |
| | TGGCGTTATAACAGGAAG | | | |
| | CAGAGTGGCTATGG | | | |
| 295 | CCAACGACTAACCCTGAAA | N | 0.004463 | 8.14E−05 |
| | TGGGGGTGTTCCAGCCTT | | | |
| | CAGCGAGATGGCC | | | |
| 296 | CAAGAGTGCCACAGATATT | N | 0.04305 | 0.000964 |
| | CTCCTGGGGGAGGATGCT | | | |
| | GGTGTTGGGAGGG | | | |
| 297 | GAACAATGGTCGTGCCAA | N | 0.020953 | 0.01742 |
| | AAAGGGCCGCGGCCACAT | | | |
| | GCAGCCTATTCGCT | | | |
| 298 | CGGCCTGATGGAGAGAAG | N | 0.008627 | 0.002771 |
| | GAACATGTTCGACTGGCT | | | |
| | CCTGATTACAATGC | | | |
| 299 | ACTCTGTAAGGAAGTTCCC | N | 0.016212 | 0.007996 |
| | AAATACAAACTTATAACCC | | | |
| | CAGCTGTGGTCT | | | |
| 300 | GTAACAGGGTGCAGTGTT | P | 0.008098 | 0.00186 |
| | GTTTATACTTCATTGCTCC | | | |
| | TTCAGGACATGGG | | | |
| 301 | TATGTCCTCTGATTGGGAC | P | 0.003643 | 0.000478 |
| | AAGGCACCTGCATTCACA | | | |
| | GGCGGCCCTGAGC | | | |
| 302 | GGCTTGGCCACCCTGCCG | N | 0.0296 | 0.0072 |
| | CTGCCCAGCCACATCCCT | | | |
| | TGGTTTTGTATTTT | | | |
| 303 | TCATGGCCGCCCTCAGAC | N | 0.005792 | 0.000672 |
| | CCCTTGTGAAGCCCAAGA | | | |
| | TCGTCAAAAAGAGA | | | |
| 304 | ATCCTCTGAGAAAACAGCC | P | 0.003984 | 0.000228 |
| | CACAGGACTGGGTCCTCC | | | |
| | TTATCCGTCTTGC | | | |
| 305 | AAATGACAAAGAGCGAGT | N | 0.028468 | 0.013861 |
| | GGCAGCTGCAATGGAAAA | | | |
| | CCCCAACTTACGGG | | | |
| 306 | AGCTCAGCGGTTACTTCG | P | 0.001345 | 0.000396 |
| | CGTGTCATCAAACCACCTC | | | |
| | TCTGGGTTGTTCG | | | |
| 307 | TAGAACTATTATTGACCAC | P | 0.009458 | 0.006369 |
| | GCCTCCTCCAAGTCCCAG | | | |
| | CGAGCCCGTGTAC | | | |
| 308 | GCACCTGCTGTAGACAGA | N | 0.007479 | 0.001297 |
| | AGACAGTATTCTGCAATGA | | | |
| | CTGAGAATGCAGT | | | |
| 309 | ACTGCTGCTTCCTACCTGC | N | 0.002691 | 0.002238 |
| | AAGACGCACAATGTATGTT | | | |
| | TCAAGGGTGAGC | | | |
| 310 | GTGCCCCTCTGTATCTTTT | N | 0.015388 | 0.006234 |
| | GAGAAGTGCGGAATAGGT | | | |
| | TGCTTCTACCACC | | | |
| 311 | TTGGGAGGCAGAGGCCGG | N | 0.013481 | 0.000858 |
| | TGGGTTGCTTTAGCTCAG | | | |
| | GAGTTGGAGACAAG | | | |
| 312 | CATATATTGCATGGAGGTA | N | 0.039958 | 0.031385 |
| | CCCCAATCTGAAGTCAGTA | | | |
| | AATGAACTAATC | | | |

TABLE 2A
143 Exemplary Positive and Negative Predictor Genes of GVHD Outcome and Housekeeping (R, reference, or HSK) genes

| | | | | | Accession No. Basic | | | |
|---|---|---|---|---|---|---|---|---|
| RNA143 Index | RNA192 Index | RNA1538 Index | ProbeID | Accession No. | (without decimal) | Gene Name | Symbol | Synonyms |
| 1 | 3 | — | 7570326 | NM_000024.4 | NM_000024 | adrenergic, | ADRB2 | B2AR; |
| | | | | | | beta-2-, | | BETA2AR; |
| | | | | | | receptor, | | BAR; |
| | | | | | | surface | | ADRBR; |
| | | | | | | (ADRB2), | | ADRB2R |
| | | | | | | mRNA. | | |
| 2 | 4 | — | 5270431 | NM_004538.3 | NM_004538 | nucleosome | NAP1L3 | MB20; |
| | | | | | | assembly | | NPL3; |
| | | | | | | protein 1-like | | MGC26312 |
| | | | | | | 3 (NAP1L3), | | |
| | | | | | | mRNA. | | |
| 3 | 5 | — | 4210754 | NM_001018069.1 | NM_001018069 | SERPINE1 | SERBP1 | CGI-55; |
| | | | | | | mRNA | | FLJ90489; |
| | | | | | | binding | | DKFZp564M2423; |
| | | | | | | protein 1 | | CHD3IP; |
| | | | | | | (SERBP1), | | PAIRBP1; |
| | | | | | | transcript | | PAI- |
| | | | | | | variant 3, | | RBP1; |
| | | | | | | mRNA. | | HABP4L |
| 4 | 6 | — | 2810255 | NM_015989.3 | NM_015989 | cysteinesulfinic | CSAD | PCAP; |
| | | | | | | acid | | MGC119355; |
| | | | | | | decarboxylase | | MGC119354; |
| | | | | | | (CSAD), | | MGC119357; |
| | | | | | | mRNA. | | CSD |
| 5 | 9 | — | 6840471 | NM_021601.3 | NM_021601 | CD79a | CD79A | MB-1; |
| | | | | | | molecule, | | IGA |
| | | | | | | immunoglobulin- | | |
| | | | | | | associated | | |
| | | | | | | alpha | | |
| | | | | | | (CD79A), | | |
| | | | | | | transcript | | |
| | | | | | | variant 2, | | |
| | | | | | | mRNA. | | |
| 6 | 10 | — | 4280743 | NM_000997.3 | NM_000997 | ribosomal | RPL37 | MGC99572 |
| | | | | | | protein L37 | | |
| | | | | | | (RPL37), | | |
| | | | | | | mRNA. | | |
| 7 | 12 | — | 4590139 | NM_006297.1 | NM_006297 | X-ray repair | XRCC1 | RCC |
| | | | | | | complementing | | |
| | | | | | | defective | | |
| | | | | | | repair in | | |
| | | | | | | Chinese | | |
| | | | | | | hamster cells | | |
| | | | | | | 1 (XRCC1), | | |
| | | | | | | mRNA. | | |
| 8 | 13 | — | 5340110 | NM_024921.2 | NM_024921 | premature | POF1B | FLJ22792; |
| | | | | | | ovarian | | POF |
| | | | | | | failure, 1B | | |
| | | | | | | (POF1B), | | |
| | | | | | | mRNA. | | |
| 9 | 14 | — | 2940048 | NM_001003789.1 | NM_001003789 | RAB, member | RABL2B | |
| | | | | | | of RAS | | |
| | | | | | | oncogene | | |
| | | | | | | family-like 2B | | |
| | | | | | | (RABL2B), | | |
| | | | | | | transcript | | |
| | | | | | | variant 1, | | |
| | | | | | | mRNA. | | |
| 10 | 17 | — | 1300671 | NM_005437.2 | NM_005437 | nuclear | NCOA4 | RFG; |
| | | | | | | receptor | | ARA70; |
| | | | | | | coactivator 4 | | DKFZp762E1112; |
| | | | | | | (NCOA4), | | PTC3; |
| | | | | | | mRNA. | | ELE1 |
| 11 | 21 | — | 7560037 | NM_133471.1 | NM_133471 | KIAA1949 | KIAA1949 | HKMT1098 |
| | | | | | | (KIAA1949), | | |
| | | | | | | mRNA. | | |
| 12 | 22 | — | 2030274 | NM_138923.1 | NM_138923 | TAF1 RNA | TAF1 | KAT4; |
| | | | | | | polymerase II, | | CCG1; |
| | | | | | | TATA box | | P250; |
| | | | | | | binding | | BA2R; |
| | | | | | | protein (TBP)- | | TAFII250; |
| | | | | | | associated | | NSCL2; |
| | | | | | | factor, | | TAF2A; |
| | | | | | | 250 kDa | | OF; |
| | | | | | | (TAF1), | | DYT3; |
| | | | | | | transcript | | CCGS |
| | | | | | | variant 2, | | |
| | | | | | | mRNA. | | |
| 13 | 24 | — | 6940088 | NM_201554.1 | NM_201554 | diacylglycerol | DGKA | MGC42356; |
| | | | | | | kinase, alpha | | DGK- |
| | | | | | | 80 kDa | | alpha; |
| | | | | | | (DGKA), | | DAGK1; |
| | | | | | | transcript | | MGC12821; |
| | | | | | | variant 4, | | DAGK |
| | | | | | | mRNA. | | |
| 14 | 25 | — | 580240 | NM_145755.1 | NM_145755 | tetratricopeptide | TTC21A | STI2; |
| | | | | | | repeat | | MGC70523; |
| | | | | | | domain 21A | | DKFZp686P18239; |
| | | | | | | (TTC21A), | | MGC156293 |
| | | | | | | mRNA. | | |
| 15 | 27 | — | 7400408 | NM_178471.1 | NM_178471 | G protein- | GPR119 | hGPCR2; |
| | | | | | | coupled | | GPCR2; |
| | | | | | | receptor 119 | | MGC119957 |
| | | | | | | (GPR119), | | |
| | | | | | | mRNA. | | |
| 16 | 29 | — | 1010224 | NM_020654.3 | NM_020654 | SUMO1/sentrin | SENP7 | KIAA1707; |
| | | | | | | specific | | MGC157730 |
| | | | | | | peptidase 7 | | |
| | | | | | | (SENP7), | | |
| | | | | | | transcript | | |
| | | | | | | variant 1, | | |
| | | | | | | mRNA. | | |
| 17 | 30 | — | 5700519 | NM_002082.2 | NM_002082 | G protein- | GRK6 | FLJ32135; |
| | | | | | | coupled | | GPRK6 |
| | | | | | | receptor | | |
| | | | | | | kinase 6 | | |
| | | | | | | (GRK6), | | |
| | | | | | | transcript | | |
| | | | | | | variant 2, | | |
| | | | | | | mRNA. | | |
| 18 | 31 | — | 7100615 | NM_001042472.1 | NM_001042472 | abhydrolase | ABHD12 | DKFZP434P106; |
| | | | | | | domain | | dJ965G21.2; |
| | | | | | | containing 12 | | C20orf22; |
| | | | | | | (ABHD12), | | ABHD12A; |
| | | | | | | transcript | | BEM46L2 |
| | | | | | | variant 1, | | |
| | | | | | | mRNA. | | |
| 19 | 32 | — | 2260615 | NM_004698.1 | NM_004698 | PRP3 pre- | PRPF3 | HPRP3P; |
| | | | | | | mRNA | | HPRP3; |
| | | | | | | processing | | Prp3p; |
| | | | | | | factor 3 | | RP18; |
| | | | | | | homolog (S. cerevisiae) | | PRP3 |
| | | | | | | (PRPF3), | | |
| | | | | | | mRNA. | | |
| 20 | 33 | — | 4200458 | NM_005249.3 | NM_005249 | forkhead box | FOXG1 | FKHL1; |
| | | | | | | G1 (FOXG1), | | KHL2; |
| | | | | | | mRNA. | | HFK3; |
| | | | | | | | | HBF2; |
| | | | | | | | | FOXG1C; |
| | | | | | | | | QIN; |
| | | | | | | | | FKHL2; |
| | | | | | | | | HBF-2; |
| | | | | | | | | HBF-1; |
| | | | | | | | | FKH2; |
| | | | | | | | | HFK1; |
| | | | | | | | | FKHL4; |
| | | | | | | | | HBF-G2; |
| | | | | | | | | BF2; |
| | | | | | | | | FHKL3; |
| | | | | | | | | BF1; |
| | | | | | | | | HFK2; |
| | | | | | | | | HBF-3; |
| | | | | | | | | FOXG1B; |
| | | | | | | | | FKHL3; |
| | | | | | | | | FOXG1A |
| 21 | 34 | — | 4810333 | NM_153701.1 | NM_153701 | interleukin 12 | IL12RB1 | CD212; |
| | | | | | | receptor, beta | | IL-12R- |
| | | | | | | 1 (IL12RB1), | | BETA1; |
| | | | | | | transcript | | IL12RB; |
| | | | | | | variant 2, | | MGC34454 |
| | | | | | | mRNA. | | |
| 22 | 36 | — | 1820035 | NM_001077268.1 | NM_001077268 | zinc finger, | ZFYVE19 | FLJ14840; |
| | | | | | | FYVE domain | | MPFYVE |
| | | | | | | containing 19 | | |
| | | | | | | (ZFYVE19), | | |
| | | | | | | mRNA. | | |
| 23 | 37 | — | 6770168 | NM_006371.3 | NM_006371 | cartilage | CRTAP | CASP; |
| | | | | | | associated | | OI7 |
| | | | | | | protein | | |
| | | | | | | (CRTAP), | | |
| | | | | | | mRNA. | | |
| 24 | 39 | — | 5360376 | NM_006762.1 | NM_006762 | lysosomal | LAPTM5 | MGC125860; |
| | | | | | | associated | | MGC125861 |
| | | | | | | multispanning | | |
| | | | | | | membrane | | |
| | | | | | | protein 5 | | |
| | | | | | | (LAPTM5), | | |
| | | | | | | mRNA. | | |
| 25 | 41 | — | 4850082 | NM_003780.3 | NM_003780 | UDP- | B4GALT2 | beta4Gal- |
| | | | | | | Gal:betaGlcNAc | | T2; |
| | | | | | | beta 1,4- | | B4Gal-T3; |
| | | | | | | galactosyltransferase, | | B4Gal-T2 |
| | | | | | | polypeptide 2 | | |
| | | | | | | (B4GALT2), | | |
| | | | | | | transcript | | |
| | | | | | | variant 2, | | |
| | | | | | | mRNA. | | |
| 26 | 42 | — | 2970017 | NM_005978.3 | NM_005978 | S100 calcium | S100A2 | S100L; |
| | | | | | | binding | | CAN19; |
| | | | | | | protein A2 | | MGC111539 |
| | | | | | | (S100A2), | | |
| | | | | | | mRNA. | | |
| 27 | 44 | — | 1110575 | NM_002494.2 | NM_002494 | NADH | NDUFC1 | MGC138266; |
| | | | | | | dehydrogenase | | KFYI; |
| | | | | | | (ubiquinone) | | MGC126847; |
| | | | | | | 1, | | MGC117464 |
| | | | | | | subcomplex | | |
| | | | | | | unknown, 1, | | |
| | | | | | | 6 kDa | | |
| | | | | | | (NDUFC1), | | |
| | | | | | | mRNA. | | |
| 28 | 45 | — | 1740382 | NM_000161.2 | NM_000161 | GTP | GCH1 | DYT5; |
| | | | | | | cyclohydrolase | | GTP-CH- |
| | | | | | | 1 (dopa- | | 1; |
| | | | | | | responsive | | GTPCH1; |
| | | | | | | dystonia) | | GCH |
| | | | | | | (GCH1), | | |
| | | | | | | transcript | | |
| | | | | | | variant 1, | | |
| | | | | | | mRNA. | | |
| 29 | 46 | — | 780184 | NM_006346.2 | NM_006346 | progesteroneimmunomodulatory | PIBF1 | RP11- |
| | | | | | | binding | | 505F3.1; |
| | | | | | | factor 1 | | KIAA1008; |
| | | | | | | (PIBF1), | | PIBF1; |
| | | | | | | mRNA. | | C13orf24 |
| 30 | 48 | — | 3610280 | NM_016446.2 | NM_016446 | chromosome | C9ORF127 | NGX6; |
| | | | | | | 9 open | | RP11- |
| | | | | | | reading frame | | 112J3.10; |
| | | | | | | 127 | | NAG-5; |
| | | | | | | (C9orf127), | | MGC120460 |
| | | | | | | mRNA. | | |
| 31 | 52 | — | 1510035 | NM_001981.2 | NM_001981 | epidermal | EPS15 | AF1P; |
| | | | | | | growth factor | | MLLT5; |
| | | | | | | receptor | | AF-1P |
| | | | | | | pathway | | |
| | | | | | | substrate 15 | | |
| | | | | | | (EPS15), | | |
| | | | | | | mRNA. | | |
| 32 | 53 | — | 6520605 | NM_005871.2 | NM_005871 | survival motor | SMNDC1 | SPF30; |
| | | | | | | neuron | | SMNR |
| | | | | | | domain | | |
| | | | | | | containing 1 | | |
| | | | | | | (SMNDC1), | | |
| | | | | | | mRNA. | | |
| 33 | 54 | — | 6250288 | NM_022474.2 | NM_022474 | membrane | MPP5 | FLJ12615; |
| | | | | | | protein, | | PALS1 |
| | | | | | | palmitoylated | | |
| | | | | | | 5 (MAGUK | | |
| | | | | | | p55 subfamily | | |
| | | | | | | member 5) | | |
| | | | | | | (MPP5), | | |
| | | | | | | mRNA. | | |
| 34 | 56 | — | 3420767 | NM_016173.3 | NM_016173 | HemKmethyltransferase | HEMK1 | FLJ22320 |
| | | | | | | family | | HEMK; |
| | | | | | | member 1 | | MTQ1 |
| | | | | | | (HEMK1), | | |
| | | | | | | mRNA. | | |
| 35 | 57 | — | 7510386 | NM_173843.1 | NM_173843 | interleukin 1 | IL1RN | ICIL-1RA; |
| | | | | | | receptor | | IRAP; IL- |
| | | | | | | antagonist | | 1ra3; |
| | | | | | | (IL1RN), | | MGC10430; |
| | | | | | | transcript | | IL1F3; |
| | | | | | | variant 4, | | IL1RA |
| | | | | | | mRNA. | | |
| 36 | 61 | — | 3610440 | NM_005360.3 | NM_005360 | v- | MAF | MGC71685 |
| | | | | | | mafmusculoaponeuroticfibrosarcoma | | |
| | | | | | | oncogene | | |
| | | | | | | homolog | | |
| | | | | | | (avian) (MAF), | | |
| | | | | | | transcript | | |
| | | | | | | variant 1, | | |
| | | | | | | mRNA. | | |
| 37 | 65 | — | 4150538 | NM_144675.1 | NM_144675 | GSG1-like | GSG1L | MGC18079; |
| | | | | | | (GSG1L), | | PRO19651 |
| | | | | | | mRNA. | | |
| 38 | 66 | — | 2450102 | NM_201438.1 | NM_201438 | periphilin 1 | PPHLN1 | HSPC206; |
| | | | | | | (PPHLN1), | | HSPC232; |
| | | | | | | transcript | | MGC48786 |
| | | | | | | variant 5, | | |
| | | | | | | mRNA. | | |
| 39 | 68 | — | 450398 | NM_004798.2 | NM_004798 | kinesin family | KIF3B | HH0048; |
| | | | | | | member3B | | KIAA0359 |
| | | | | | | (KIF3B), | | |
| | | | | | | mRNA. | | |
| 40 | 69 | — | 4810458 | NM_181309.1 | NM_181309 | interleukin 22 | IL22RA2 | CRF2-S1; |
| | | | | | | receptor, | | MGC150509; |
| | | | | | | alpha 2 | | IL- |
| | | | | | | (IL22RA2), | | 22BP; |
| | | | | | | transcript | | MGC150510; |
| | | | | | | variant 2, | | CRF2- |
| | | | | | | mRNA. | | 10; |
| | | | | | | | | CRF2X |
| 41 | 70 | — | 3890196 | NM_152850.2 | NM_152850 | phosphatidylinositol | PIGO | RP11- |
| | | | | | | glycan | | 182N22.4; |
| | | | | | | anchor | | DKFZp434M222; |
| | | | | | | biosynthesis, | | FLJ00135; |
| | | | | | | class O | | MGC3079; |
| | | | | | | (PIGO), | | MGC20536 |
| | | | | | | transcript | | |
| | | | | | | variant 2, | | |
| | | | | | | mRNA. | | |
| 42 | 71 | — | 1230156 | NM_004155.3 | NM_004155 | serpin | SERPINB9 | PI9; CAP- |
| | | | | | | peptidase | | 3; CAP3 |
| | | | | | | inhibitor, clade | | |
| | | | | | | B (ovalbumin), | | |
| | | | | | | member 9 | | |
| | | | | | | (SERPINB9), | | |
| | | | | | | mRNA. | | |
| 43 | 72 | — | 1190138 | NM_003328.2 | NM_003328 | TXK tyrosine | TXK | MGC22473; |
| | | | | | | kinase (TXK), | | PSCTK5; |
| | | | | | | mRNA. | | PTK4; |
| | | | | | | | | BTKL; |
| | | | | | | | | TKL; RLK |
| 44 | 73 | — | 3780139 | NM_020820.3 | NM_020820 | phosphatidylinositol | PREX1 | KIAA1415 |
| | | | | | | 3,4,5- | | |
| | | | | | | trisphosphate- | | |
| | | | | | | dependent | | |
| | | | | | | RAC | | |
| | | | | | | exchanger 1 | | |
| | | | | | | (PREX1), | | |
| | | | | | | mRNA. | | |
| 45 | 75 | — | 5340458 | NM_018044.2 | NM_018044 | NOL1/NOP2/ | NSUN5 | p120; |
| | | | | | | Sun domain | | FLJ10267; |
| | | | | | | family, | | WBSCR20; |
| | | | | | | member 5 | | p120 |
| | | | | | | (NSUN5), | | (NOL1); |
| | | | | | | transcript | | MGC986; |
| | | | | | | variant 2, | | WBSCR20A; |
| | | | | | | mRNA. | | (NOL1); |
| | | | | | | | | NOL1R; |
| | | | | | | | | NSUN5A |
| 46 | 76 | — | 70608 | NM_172177.1 | NM_172177 | mitochondrial | MRPL42 | PTD007; |
| | | | | | | ribosomal | | MRPS32; |
| | | | | | | protein L42 | | MRP-L31; |
| | | | | | | (MRPL42), | | RPML31; |
| | | | | | | nuclear gene | | HSPC204 |
| | | | | | | encoding | | |
| | | | | | | mitochondrial | | |
| | | | | | | protein, | | |
| | | | | | | transcript | | |
| | | | | | | variant 2, | | |
| | | | | | | mRNA. | | |
| 47 | 77 | — | 3460053 | NM_020808.3 | NM_020808 | signal-induced | SIPA1L2 | SPAL2; |
| | | | | | | proliferation- | | FLJ23126; |
| | | | | | | associated 1 | | KIAA1389; |
| | | | | | | like 2 | | FLJ23632 |
| | | | | | | (SIPA1L2), | | |
| | | | | | | mRNA. | | |
| 48 | 78 | — | 6220343 | NM_021098.2 | NM_021098 | calcium | CACNA1H | CACNA1HB; |
| | | | | | | channel, | | FLJ90484; |
| | | | | | | voltage- | | Cav3.2 |
| | | | | | | dependent, T | | |
| | | | | | | type, alpha 1H | | |
| | | | | | | subunit | | |
| | | | | | | (CACNA1H), | | |
| | | | | | | transcript | | |
| | | | | | | variant 1, | | |
| | | | | | | mRNA. | | |
| 49 | 79 | — | 6620753 | NM_006007.1 | NM_006007 | zinc finger, | ZFAND5 | ZFAND5A; |
| | | | | | | AN1-type | | ZA20D2; |
| | | | | | | domain 5 | | ZNF216 |
| | | | | | | (ZFAND5), | | |
| | | | | | | mRNA. | | |
| 50 | 80 | — | 5860605 | NM_013374.3 | NM_013374 | programmed | PDCD6IP | MGC17003; |
| | | | | | | cell death 6 | | Alix; |
| | | | | | | interacting | | DRIP4; |
| | | | | | | protein | | AIP1; |
| | | | | | | (PDCD6IP), | | HP95 |
| | | | | | | mRNA. | | |
| 51 | 81 | — | 160390 | NM_001014839.1 | NM_001014839 | neurochondrin | NCDN | KIAA0607 |
| | | | | | | (NCDN), | | |
| | | | | | | transcript | | |
| | | | | | | variant 1, | | |
| | | | | | | mRNA. | | |
| 52 | 82 | — | 3890136 | NM_006370.1 | NM_006370 | vesicle | VTI1B | VTI1; |
| | | | | | | transport | | VTI1L; |
| | | | | | | through | | VTI2 |
| | | | | | | interaction | | |
| | | | | | | with t- | | |
| | | | | | | SNAREs | | |
| | | | | | | homolog 1B | | |
| | | | | | | (yeast) | | |
| | | | | | | (VTI1B), | | |
| | | | | | | mRNA. | | |
| 53 | 84 | — | 2750592 | NM_032026.2 | NM_032026 | TatDDNase | TATDN1 | CDA11 |
| | | | | | | domain | | |
| | | | | | | containing 1 | | |
| | | | | | | (TATDN1), | | |
| | | | | | | mRNA. | | |
| 54 | 85 | — | 7330435 | NM_005436.2 | NM_005436 | coiled-coil | CCDC6 | TST1; |
| | | | | | | domain | | TPC; |
| | | | | | | containing 6 | | PTC; H4; |
| | | | | | | (CCDC6), | | D10S170; |
| | | | | | | mRNA. | | FLJ32286 |
| 55 | 86 | — | 7570500 | NM_032314.3 | NM_032314 | coenzyme Q5 | COQ5 | MGC4767; |
| | | | | | | homolog, | | MGC104303 |
| | | | | | | methyltransferase | | |
| | | | | | | (S. cerevisiae) | | |
| | | | | | | (COQ5), | | |
| | | | | | | mRNA. | | |
| 56 | 87 | — | 6020612 | NM_002158.3 | NM_002158 | forkhead box | FOXN2 | HTLF |
| | | | | | | N2 (FOXN2), | | |
| | | | | | | mRNA. | | |
| 57 | 88 | — | 840240 | NM_007124.2 | NM_007124 | utrophin | UTRN | DRP; |
| | | | | | | (UTRN), | | DMDL; |
| | | | | | | mRNA. | | DRP1; |
| | | | | | | | | FLJ23678 |
| 58 | 89 | — | 6450176 | NM_138711.3 | NM_138711 | peroxisome | PPARG | NR1C3; |
| | | | | | | proliferator- | | PPARG1; |
| | | | | | | activated | | PPARG2 |
| | | | | | | receptor | | |
| | | | | | | gamma | | |
| | | | | | | (PPARG), | | |
| | | | | | | transcript | | |
| | | | | | | variant 3, | | |
| | | | | | | mRNA. | | |
| 59 | 90 | — | 4060669 | NM_019083.1 | NM_019083 | coiled-coil | CCDC76 | FLJ10287; |
| | | | | | | domain | | FLJ11219 |
| | | | | | | containing 76 | | |
| | | | | | | (CCDC76), | | |
| | | | | | | mRNA. | | |
| 60 | 91 | — | 10220 | NM_001002246.1 | NM_001002246 | APC11 | ANAPC11 | HSPC214; |
| | | | | | | anaphase | | MGC882; |
| | | | | | | promoting | | Apc11p; |
| | | | | | | complex | | APC11 |
| | | | | | | subunit 11 | | |
| | | | | | | homolog | | |
| | | | | | | (yeast) | | |
| | | | | | | (ANAPC11), | | |
| | | | | | | transcript | | |
| | | | | | | variant 4, | | |
| | | | | | | mRNA. | | |
| 61 | 92 | — | 770541 | NM_001007277.1 | NM_001007277 | etoposide | EI24 | TP53I8; |
| | | | | | | induced 2.4 | | PIG8 |
| | | | | | | mRNA (EI24), | | |
| | | | | | | transcript | | |
| | | | | | | variant 2, | | |
| | | | | | | mRNA. | | |
| 62 | 93 | — | 7160270 | NM_004450.1 | NM_004450 | enhancer of | ERH | FLJ27340; |
| | | | | | | rudimentary | | DROER |
| | | | | | | homolog | | |
| | | | | | | (Drosophila) | | |
| | | | | | | (ERH), | | |
| | | | | | | mRNA. | | |
| 63 | 94 | — | 730497 | NM_032449.1 | NM_032449 | coiled-coil and | CC2D1B | RP11- |
| | | | | | | C2 domain | | 155O18.2; |
| | | | | | | containing 1B | | KIAA1836 |
| | | | | | | (CC2D1B), | | |
| | | | | | | mRNA. | | |
| 64 | 95 | — | 4220220 | NM_001009922.1 | NM_001009922 | ring finger and | RCHY1 | ARNIP; |
| | | | | | | CHY zinc | | PRO1996; |
| | | | | | | finger domain | | CHIMP; |
| | | | | | | containing 1 | | DKFZp586C1620; |
| | | | | | | (RCHY1), | | ZNF363; |
| | | | | | | transcript | | hARNIP; |
| | | | | | | variant 3, | | PIRH2; |
| | | | | | | mRNA. | | RNF199 |
| 65 | 96 | — | 1990653 | NM_006405.5 | NM_006405 | transmembrane 9 | TM9SF1 | HMP70; |
| | | | | | | superfamily | | MP70 |
| | | | | | | member 1 | | |
| | | | | | | (TM9SF1), | | |
| | | | | | | transcript | | |
| | | | | | | variant 1, | | |
| | | | | | | mRNA. | | |
| 66 | 97 | — | 5490717 | NM_198585.2 | NM_198585 | ectonucleoside | ENTPD8 | GLSR2492; |
| | | | | | | triphosphate | | UNQ2492; |
| | | | | | | diphosphohydrolase 8 | | NTPDase-8 |
| | | | | | | (ENTPD8), | | |
| | | | | | | transcript | | |
| | | | | | | variant 2, | | |
| | | | | | | mRNA. | | |
| 67 | 98 | — | 1010739 | NM_033364.3 | NM_033364 | chromosome | C3ORF15 | AAT1alpha; |
| | | | | | | 3 open | | AAT1; |
| | | | | | | reading frame | | DKFZp781A2221 |
| | | | | | | 15 (C3orf15), | | |
| | | | | | | mRNA. | | |
| 68 | 99 | — | 1340681 | NM_015633.1 | NM_015633 | FGFR1 | FGFR1OP2 | DKFZp564O1863; |
| | | | | | | oncogene | | HSPC123- |
| | | | | | | partner 2 | | like |
| | | | | | | (FGFR1OP2), | | |
| | | | | | | mRNA. | | |
| 69 | 100 | — | 2680497 | NM_014865.2 | NM_014865 | non- | NCAPD2 | hCAP-D2; |
| | | | | | | SMCcondensin | | KIAA0159; |
| | | | | | | I complex, | | CAP-D2; |
| | | | | | | subunit D2 | | CNAP1 |
| | | | | | | (NCAPD2), | | |
| | | | | | | mRNA. | | |
| 70 | 101 | — | 830440 | NM_003268.4 | NM_003268 | toll-like | TLR5 | MGC126430; |
| | | | | | | receptor 5 | | SLEB1; |
| | | | | | | (TLR5), | | MGC126431; |
| | | | | | | mRNA. | | FLJ10052; |
| | | | | | | | | TIL3 |
| 71 | 103 | — | 3800253 | NM_172388.1 | NM_172388 | nuclear factor | NFATC1 | NFATc; |
| | | | | | | of activated T- | | MGC138448; |
| | | | | | | cells, | | NFAT2; |
| | | | | | | cytoplasmic, | | NF-ATC |
| | | | | | | calcineurin- | | |
| | | | | | | dependent 1 | | |
| | | | | | | (NFATC1), | | |
| | | | | | | transcript | | |
| | | | | | | variant 4, | | |
| | | | | | | mRNA. | | |
| 72 | 104 | — | 2000669 | NM_024605.3 | NM_024605 | Rho GTPase | ARHGAP10 | FLJ20896; |
| | | | | | | activating | | PS-GAP; |
| | | | | | | protein 10 | | GRAF2; |
| | | | | | | (ARHGAP10), | | FLJ41791 |
| | | | | | | mRNA. | | |
| 73 | 105 | — | 6290181 | NM_058192.2 | NM_058192 | RNA | RPUSD1 | MGC19600; |
| | | | | | | pseudouridylate | | RLUCL; |
| | | | | | | synthase | | C16orf40 |
| | | | | | | domain | | |
| | | | | | | containing 1 | | |
| | | | | | | (RPUSD1), | | |
| | | | | | | mRNA. | | |
| 74 | 107 | — | 7050291 | NM_024756.1 | NM_024756 | multimerin 2 | MMRN2 | EndoGlyx- |
| | | | | | | (MMRN2), | | 1; |
| | | | | | | mRNA. | | FLJ13465; |
| | | | | | | | | EMILIN3; |
| | | | | | | | | ENDOGLYX1 |
| 75 | 108 | — | 3370280 | NM_016447.2 | NM_016447 | membrane | MPP6 | p55T; |
| | | | | | | protein, | | VAM-1; |
| | | | | | | palmitoylated | | VAM1; |
| | | | | | | 6 (MAGUK | | PALS2 |
| | | | | | | p55 subfamily | | |
| | | | | | | member 6) | | |
| | | | | | | (MPP6), | | |
| | | | | | | mRNA. | | |
| 76 | 109 | — | 1050040 | NM_004925.3 | NM_004925 | aquaporin 3 | AQP3 | GIL |
| | | | | | | (Gill blood | | |
| | | | | | | group) | | |
| | | | | | | (AQP3), | | |
| | | | | | | mRNA. | | |
| 77 | 110 | — | 6580379 | NM_006348.2 | NM_006348 | component of | COG5 | GOLTC1; |
| | | | | | | oligomericgolgi | | GTC90 |
| | | | | | | complex 5 | | |
| | | | | | | (COG5), | | |
| | | | | | | transcript | | |
| | | | | | | variant 1, | | |
| | | | | | | mRNA. | | |
| 78 | 111 | — | 730487 | NM_020320.2 | NM_020320 | arginyl- | RARS2 | dJ382I10.6; |
| | | | | | | tRNAsynthetase | | DALRD2; |
| | | | | | | 2, | | MGC14993; |
| | | | | | | mitochondrial | | PCH6; |
| | | | | | | (RARS2), | | PRO1992; |
| | | | | | | nuclear gene | | RARSL; |
| | | | | | | encoding | | MGC23778 |
| | | | | | | mitochondrial | | |
| | | | | | | protein, | | |
| | | | | | | mRNA. | | |
| 79 | 112 | — | 2140682 | NM_175617.2 | NM_175617 | metallothionein | MT1E | MT1; |
| | | | | | | 1E | | MTD |
| | | | | | | (functional) | | |
| | | | | | | (MT1E), | | |
| | | | | | | mRNA. | | |
| 80 | 113 | — | 2650148 | NM_018268.2 | NM_018268 | WD repeat | WDR41 | MSTP048; |
| | | | | | | domain 41 | | FLJ10904 |
| | | | | | | (WDR41), | | |
| | | | | | | mRNA. | | |
| 81 | 115 | — | 3130471 | NM_198465.2 | NM_198465 | Nik related | NRK | DKFZp686A17109; |
| | | | | | | kinase (NRK), | | FLJ16788; |
| | | | | | | mRNA. | | NESK; |
| | | | | | | | | MGC131849 |
| 82 | 116 | — | 4810274 | NM_199367.1 | NM_199367 | spastic | SPG7 | CAR; |
| | | | | | | paraplegia 7 | | FLJ37308; |
| | | | | | | (pure and | | SPG5C; |
| | | | | | | complicated | | MGC126332; |
| | | | | | | autosomal | | CMAR; |
| | | | | | | recessive) | | MGC126331; |
| | | | | | | (SPG7), | | PGN |
| | | | | | | nuclear gene | | |
| | | | | | | encoding | | |
| | | | | | | mitochondrial | | |
| | | | | | | protein, | | |
| | | | | | | transcript | | |
| | | | | | | variant 2, | | |
| | | | | | | mRNA. | | |
| 83 | 119 | — | 5670100 | NM_000355.2 | NM_000355 | transcobalamin | TCN2 | D22S750; |
| | | | | | | II; | | TC2; |
| | | | | | | macrocytic | | D22S676 |
| | | | | | | anemia | | |
| | | | | | | (TCN2), | | |
| | | | | | | mRNA. | | |
| 84 | 121 | — | 7320441 | NM_013332.3 | NM_013332 | hypoxia- | HIG2 | FLJ21076; |
| | | | | | | inducible | | MGC138388 |
| | | | | | | protein 2 | | |
| | | | | | | (HIG2), | | |
| | | | | | | transcript | | |
| | | | | | | variant 1, | | |
| | | | | | | mRNA. | | |
| 85 | 123 | — | 2060674 | NM_000067.1 | NM_000067 | carbonic | CA2 | Car2; CA- |
| | | | | | | anhydrase II | | II; CAII; |
| | | | | | | (CA2), mRNA. | | CA II |
| 86 | 124 | — | 6350671 | NM_023080.1 | NM_023080 | chromosome | C8ORF33 | FLJ20989 |
| | | | | | | 8 open | | |
| | | | | | | reading frame | | |
| | | | | | | 33 (C8orf33), | | |
| | | | | | | mRNA. | | |
| 87 | 125 | — | 770619 | NM_003473.2 | NM_003473 | signal | STAM | DKFZp686J2352; |
| | | | | | | transducing | | STAM1 |
| | | | | | | adaptor | | |
| | | | | | | molecule | | |
| | | | | | | (SH3 domain | | |
| | | | | | | and ITAM | | |
| | | | | | | motif) 1 | | |
| | | | | | | (STAM), | | |
| | | | | | | mRNA. | | |
| 88 | 126 | — | 540452 | NM_022743.1 | NM_022743 | SET and | SMYD3 | bA74P14.1; |
| | | | | | | MYND | | ZMYND1; |
| | | | | | | domain | | ZNFN3A1; |
| | | | | | | containing 3 | | FLJ21080; |
| | | | | | | (SMYD3), | | MGC104324 |
| | | | | | | mRNA. | | |
| 89 | 127 | — | 4200441 | NM_003003.2 | NM_003003 | SEC14-like 1 | SEC14L1 | SEC14L; |
| | | | | | | (S. cerevisiae) | | DKFZp686C06176; |
| | | | | | | (SEC14L1), | | PRELID4A |
| | | | | | | transcript | | |
| | | | | | | variant 1, | | |
| | | | | | | mRNA. | | |
| 90 | 128 | — | 6550279 | NM_000848.2 | NM_000848 | glutathione S- | GSTM2 | GST4; |
| | | | | | | transferase | | GSTM; |
| | | | | | | M2 (muscle) | | GSTM2-2; |
| | | | | | | (GSTM2), | | GTHMUS; |
| | | | | | | mRNA. | | MGC117303 |
| 91 | 129 | — | 4860392 | NM_178128.3 | NM_178128 | fatty acid | FADS6 | FP18279 |
| | | | | | | desaturase | | |
| | | | | | | domain family, | | |
| | | | | | | member 6 | | |
| | | | | | | (FADS6), | | |
| | | | | | | mRNA. | | |
| 92 | 130 | — | 3940735 | NM_003093.1 | NM_003093 | small nuclear | SNRPC | FLJ20302 |
| | | | | | | ribonucleoprotein | | |
| | | | | | | polypeptide C | | |
| | | | | | | (SNRPC), | | |
| | | | | | | mRNA. | | |
| 93 | 131 | — | 1850347 | NM_021067.3 | NM_021067 | GINS | GINS1 | PSF1; |
| | | | | | | complex | | KIAA0186; |
| | | | | | | subunit 1 | | RP4- |
| | | | | | | (Psf1 | | 691N24.2 |
| | | | | | | homolog) | | |
| | | | | | | (GINS1), | | |
| | | | | | | mRNA. | | |
| 94 | 133 | — | 7000703 | NM_016310.2 | NM_016310 | polymerase | POLR3K | C11; |
| | | | | | | (RNA) III | | RPC10; |
| | | | | | | (DNA | | C11- |
| | | | | | | directed) | | RNP3; |
| | | | | | | polypeptide K, | | My010; |
| | | | | | | 12.3 kDa | | RPC11; |
| | | | | | | (POLR3K), | | hRPC11 |
| | | | | | | mRNA. | | |
| 95 | 134 | — | 2690315 | NM_014901.4 | NM_014901 | ring finger | RNF44 | KIAA1100 |
| | | | | | | protein 44 | | |
| | | | | | | (RNF44), | | |
| | | | | | | mRNA. | | |
| 96 | 135 | — | 4900670 | NM_004255.2 | NM_004255 | cytochrome c | COX5A | COX-VA; |
| | | | | | | oxidase | | VA; COX |
| | | | | | | subunit Va | | |
| | | | | | | (COX5A), | | |
| | | | | | | nuclear gene | | |
| | | | | | | encoding | | |
| | | | | | | mitochondrial | | |
| | | | | | | protein, | | |
| | | | | | | mRNA. | | |
| 97 | 136 | — | 6900014 | NM_032177.2 | NM_032177 | RNA U, small | RNUXA | FLJ13193; |
| | | | | | | nuclear RNA | | PHAX |
| | | | | | | export adaptor | | |
| | | | | | | (phosphorylation | | |
| | | | | | | regulated) | | |
| | | | | | | (RNUXA), | | |
| | | | | | | mRNA. | | |
| 98 | 138 | — | 5570338 | NM_182922.2 | NM_182922 | HEAT repeat | HEATR3 | FLJ20718 |
| | | | | | | containing 3 | | |
| | | | | | | (HEATR3), | | |
| | | | | | | mRNA. | | |
| 99 | 139 | — | 4540241 | NM_032412.3 | NM_032412 | chromosome | C5ORF32 | ORF1- |
| | | | | | | 5 open | | FL49 |
| | | | | | | reading frame | | |
| | | | | | | 32 (C5orf32), | | |
| | | | | | | mRNA. | | |
| 100 | 140 | — | 2850360 | NM_001707.2 | NM_001707 | B-cell | BCL7B | |
| | | | | | | CLL/lymphoma | | |
| | | | | | | 7B | | |
| | | | | | | (BCL7B), | | |
| | | | | | | mRNA. | | |
| 101 | 141 | — | 6860653 | NM_006402.2 | NM_006402 | hepatitis B | HBXIP | MGC71071; |
| | | | | | | virus x | | XIP |
| | | | | | | interacting | | |
| | | | | | | protein | | |
| | | | | | | (HBXIP), | | |
| | | | | | | mRNA. | | |
| 102 | 142 | — | 2350209 | NM_139118.1 | NM_139118 | YY1 | YY1AP1 | YAP; |
| | | | | | | associated | | YY1AP; |
| | | | | | | protein 1 | | HCCA2; |
| | | | | | | (YY1AP1), | | FLJ10875; |
| | | | | | | transcript | | FLJ13914; |
| | | | | | | variant 2, | | HCCA1 |
| | | | | | | mRNA. | | |
| 103 | 143 | — | 3940754 | NM_006566.1 | NM_006566 | CD226 | CD226 | TLiSA1; |
| | | | | | | molecule | | PTA1; |
| | | | | | | (CD226), | | DNAM1; |
| | | | | | | mRNA. | | DNAM-1 |
| 104 | 144 | — | 6650747 | NM_152320.1 | NM_152320 | zinc finger | ZNF641 | FLJ31295; |
| | | | | | | protein 641 | | DKFZp667D1012 |
| | | | | | | (ZNF641), | | |
| | | | | | | mRNA. | | |
| 105 | 145 | — | 3780400 | NM_014212.3 | NM_014212 | homeobox | HOXC11 | HOX3H; |
| | | | | | | C11 | | MGC4906 |
| | | | | | | (HOXC11), | | |
| | | | | | | mRNA. | | |
| 106 | 146 | — | 6770017 | NM_007249.4 | NM_007249 | Kruppel-like | KLF12 | AP-2rep; |
| | | | | | | factor 12 | | AP2REP; |
| | | | | | | (KLF12), | | HSPC122 |
| | | | | | | mRNA. | | |
| 107 | 147 | — | 2340059 | NM_024516.2 | NM_024516 | chromosome | C16ORF53 | PA1; |
| | | | | | | 16 open | | MGC4606 |
| | | | | | | reading frame | | |
| | | | | | | 53 (C16orf53), | | |
| | | | | | | mRNA. | | |
| 108 | 148 | — | 4150593 | NM_015077.2 | NM_015077 | sterile alpha | SARM1 | SAMD2; |
| | | | | | | and TIR motif | | KIAA0524; |
| | | | | | | containing 1 | | SARM; |
| | | | | | | (SARM1), | | FLJ36296 |
| | | | | | | mRNA. | | |
| 109 | 149 | — | 2650408 | NM_018177.2 | NM_018177 | Nedd4 | N4BP2 | B3BP; |
| | | | | | | binding | | KIAA1413; |
| | | | | | | protein 2 | | FLJ10680 |
| | | | | | | (N4BP2), | | |
| | | | | | | mRNA. | | |
| 110 | 150 | — | 10435 | NM_001001660.2 | NM_001001660 | LYR motif | LYRM5 | |
| | | | | | | containing 5 | | |
| | | | | | | (LYRM5), | | |
| | | | | | | mRNA. | | |
| 111 | 151 | — | 2690528 | NM_004169.3 | NM_004169 | serinehydroxy | SHMT1 | MGC15229; |
| | | | | | | methyltransferase 1 | | MGC24556; |
| | | | | | | (soluble) | | SHMT; |
| | | | | | | (SHMT1), | | CSHMT |
| | | | | | | transcript | | |
| | | | | | | variant 1, | | |
| | | | | | | mRNA. | | |
| 112 | 152 | — | 130093 | NM_005951.2 | NM_005951 | metallothionein | MT1H | MGC70702; |
| | | | | | | 1H (MT1H), | | MT1 |
| | | | | | | mRNA. | | |
| 113 | 153 | — | 1010692 | NM_005234.3 | NM_005234 | nuclear | NR2F6 | EAR-2; |
| | | | | | | receptor | | EAR2; |
| | | | | | | subfamily 2, | | ERBAL2 |
| | | | | | | group F, | | |
| | | | | | | member 6 | | |
| | | | | | | (NR2F6), | | |
| | | | | | | mRNA. | | |
| 114 | 156 | — | 520431 | NM_014819.3 | NM_014819 | praja 2, RING- | PJA2 | KIAA0438; |
| | | | | | | H2 motif | | RNF131; |
| | | | | | | containing | | Neurodap1 |
| | | | | | | (PJA2), | | |
| | | | | | | mRNA. | | |
| 115 | 157 | — | 5560079 | NM_001077191.1 | NM_001077191 | G protein- | GPBAR1 | GPR131; |
| | | | | | | coupled bile | | M-BAR; |
| | | | | | | acid receptor | | GPCR; |
| | | | | | | 1 (GPBAR1), | | GPCR19; |
| | | | | | | transcript | | BG37; |
| | | | | | | variant 1, | | TGR5; |
| | | | | | | mRNA. | | MGC40597 |
| 116 | 158 | — | 670026 | NM_015986.2 | NM_015986 | cytokine | CRLF3 | CREME9; |
| | | | | | | receptor-like | | FRWS; |
| | | | | | | factor 3 | | CYTOR4; |
| | | | | | | (CRLF3), | | MGC20661 |
| | | | | | | mRNA. | | |
| 117 | 163 | — | 1770593 | NM_003956.3 | NM_003956 | cholesterol | CH25H | C25H |
| | | | | | | 25- | | |
| | | | | | | hydroxylase | | |
| | | | | | | (CH25H), | | |
| | | | | | | mRNA. | | |
| 118 | 166 | — | 1260438 | NM_001556.1 | NM_001556 | inhibitor of | IKBKB | IKK-beta; |
| | | | | | | kappa light | | NFKBIKB; |
| | | | | | | polypeptide | | IKK2; |
| | | | | | | gene | | FLJ40509; |
| | | | | | | enhancer in | | IKKB; |
| | | | | | | B-cells, kinase | | MGC131801 |
| | | | | | | beta (IKBKB), | | |
| | | | | | | mRNA. | | |
| 119 | 167 | — | 4050768 | NM_152889.1 | NM_152889 | carbohydrate | CHST13 | MGC119279; |
| | | | | | | (chondroitin 4) | | MGC119281; |
| | | | | | | sulfotransferase | | MGC119278; |
| | | | | | | 13 | | C4ST3 |
| | | | | | | (CHST13), | | |
| | | | | | | mRNA. | | |
| 120 | 168 | — | 1340349 | NM_001042588.1 | NM_001042588 | snurportin 1 | SNUPN | RNUT1; |
| | | | | | | (SNUPN), | | Snurportin1; |
| | | | | | | transcript | | KPNBL |
| | | | | | | variant 3, | | |
| | | | | | | mRNA. | | |
| 121 | 169 | — | 6940431 | NM_015253.1 | NM_015253 | WSC domain | WSCD1 | KIAA0523 |
| | | | | | | containing 1 | | |
| | | | | | | (WSCD1), | | |
| | | | | | | mRNA. | | |
| 122 | 170 | — | 3610241 | NM_000981.3 | NM_000981 | ribosomal | RPL19 | MGC71997; |
| | | | | | | protein L19 | | DKFZp779D216; |
| | | | | | | (RPL19), | | FLJ27452 |
| | | | | | | mRNA. | | |
| 123 | 171 | — | 6580577 | NM_031369.2 | NM_031369 | heterogeneous | HNRNPD | P37; |
| | | | | | | nuclear | | AUF1; |
| | | | | | | ribonucleoprotein | | hnRNPD0; |
| | | | | | | D (AU-rich | | AUF1A |
| | | | | | | element RNA | | |
| | | | | | | binding | | |
| | | | | | | protein 1, | | |
| | | | | | | 37 kDa) | | |
| | | | | | | (HNRNPD), | | |
| | | | | | | transcript | | |
| | | | | | | variant 2, | | |
| | | | | | | mRNA. | | |
| 124 | 172 | — | 3360228 | NM_001023.2 | NM_001023 | ribosomal | RPS20 | MGC102930; |
| | | | | | | protein S20 | | FLJ27451 |
| | | | | | | (RPS20), | | |
| | | | | | | mRNA. | | |
| 125 | 173 | — | 6130390 | NM_016093.2 | NM_016093 | ribosomal | RPL26L1 | RPL26P1; |
| | | | | | | protein L26- | | FLJ46904 |
| | | | | | | like 1 | | |
| | | | | | | (RPL26L1), | | |
| | | | | | | mRNA. | | |
| 126 | 175 | — | 1110017 | NM_032195.1 | NM_032195 | SON DNA | SON | FLJ21099; |
| | | | | | | binding | | SON3; |
| | | | | | | protein (SON), | | KIAA1019; |
| | | | | | | transcript | | BASS1; |
| | | | | | | variant b, | | NREBP; |
| | | | | | | mRNA. | | C21orf50; |
| | | | | | | | | DBP-5; |
| | | | | | | | | FLJ33914 |
| 127 | 176 | — | 2680097 | NM_016061.1 | NM_016061 | yippee-like 5 | YPEL5 | CGI-127 |
| | | | | | | (Drosophila) | | |
| | | | | | | (YPEL5), | | |
| | | | | | | mRNA. | | |
| 128 | 177 | — | 2480364 | NM_013379.2 | NM_013379 | dipeptidyl- | DPP7 | DPP2; |
| | | | | | | peptidase 7 | | DPPII; |
| | | | | | | (DPP7), | | QPP |
| | | | | | | mRNA. | | |
| 129 | 178 | — | 6330044 | NM_004034.1 | NM_004034 | annexin A7 | ANXA7 | ANX7; |
| | | | | | | (ANXA7), | | SNX |
| | | | | | | transcript | | |
| | | | | | | variant 2, | | |
| | | | | | | mRNA. | | |
| 130 | 179 | — | 240725 | NM_001033112.1 | NM_001033112 | poly(A) | PAIP2 | PAIP2A; |
| | | | | | | binding | | MGC72018 |
| | | | | | | protein | | |
| | | | | | | interacting | | |
| | | | | | | protein 2 | | |
| | | | | | | (PAIP2), | | |
| | | | | | | transcript | | |
| | | | | | | variant 1, | | |
| | | | | | | mRNA. | | |
| 131 | 180 | — | 3390192 | NM_006861.4 | NM_006861 | RAB35, | RAB35 | RAB1C; |
| | | | | | | member RAS | | H-ray; |
| | | | | | | oncogene | | RAY |
| | | | | | | family | | |
| | | | | | | (RAB35), | | |
| | | | | | | mRNA. | | |
| 132 | 181 | — | 4150670 | NM_007065.3 | NM_007065 | cell division | CDC37 | P50CDC37 |
| | | | | | | cycle 37 | | |
| | | | | | | homolog (S. cerevisiae) | | |
| | | | | | | (CDC37), | | |
| | | | | | | mRNA. | | |
| 133 | 182 | — | 7200037 | NM_005626.3 | NM_005626 | splicing factor, | SFRS4 | SRP75 |
| | | | | | | arginine/serine- | | |
| | | | | | | rich 4 | | |
| | | | | | | (SFRS4), | | |
| | | | | | | mRNA. | | |
| 134 | 183 | — | 5690202 | NM_018064.2 | NM_018064 | chromosome | C6ORF166 | FLJ10342; |
| | | | | | | 6 open | | dJ486L4.2 |
| | | | | | | reading frame | | |
| | | | | | | 166 | | |
| | | | | | | (C6orf166), | | |
| | | | | | | mRNA. | | |
| 135 | 184 | — | 3830538 | NM_030818.2 | NM_030818 | coiled-coil | CCDC130 | MGC10471 |
| | | | | | | domain | | |
| | | | | | | containing | | |
| | | | | | | 130 | | |
| | | | | | | (CCDC130), | | |
| | | | | | | mRNA. | | |
| 136 | 185 | — | 2490066 | NM_006110.1 | NM_006110 | CD2 | CD2BP2 | FWP010; |
| | | | | | | (cytoplasmic | | LIN1; |
| | | | | | | tail) binding | | Snu40 |
| | | | | | | protein 2 | | |
| | | | | | | (CD2BP2), | | |
| | | | | | | mRNA. | | |
| 137 | 186 | — | 4230050 | NM_006327.2 | NM_006327 | translocase of | TIMM23 | PRO1197; |
| | | | | | | inner | | TIMM23B; |
| | | | | | | mitochondrial | | MGC22767; |
| | | | | | | membrane 23 | | TIM23 |
| | | | | | | homolog | | |
| | | | | | | (yeast) | | |
| | | | | | | (TIMM23), | | |
| | | | | | | nuclear gene | | |
| | | | | | | encoding | | |
| | | | | | | mitochondrial | | |
| | | | | | | protein, | | |
| | | | | | | mRNA. | | |
| 138 | 187 | — | 7200598 | NM_005466.2 | NM_005466 | mediator | MED6 | NY-REN- |
| | | | | | | complex | | 28 |
| | | | | | | subunit 6 | | |
| | | | | | | (MED6), | | |
| | | | | | | mRNA. | | |
| 139 | 188 | — | 6110477 | NM_006600.2 | NM_006600 | nuclear | NUDC | NPD011; |
| | | | | | | distribution | | HNUDC; |
| | | | | | | gene C | | MNUDC |
| | | | | | | homolog (A. nidulans) | | |
| | | | | | | (NUDC), | | |
| | | | | | | mRNA. | | |
| 140 | 189 | — | 3130241 | NM_020141.3 | NM_020141 | chromosome | C1ORF119 | AD-020; |
| | | | | | | 1 open | | FLJ90710 |
| | | | | | | reading frame | | |
| | | | | | | 119 | | |
| | | | | | | (C1orf119), | | |
| | | | | | | mRNA. | | |
| 141 | 190 | — | 60390 | NM_030914.1 | NM_030914 | ubiquitin | URM1 | C9orf74; |
| | | | | | | related | | RP11- |
| | | | | | | modifier 1 | | 339B21.4; |
| | | | | | | homolog (S. cerevisiae) | | MGC2668 |
| | | | | | | (URM1), | | |
| | | | | | | mRNA. | | |
| 142 | 191 | — | 1450537 | NM_014607.3 | NM_014607 | UBX domain | UBXD2 | erasin; |
| | | | | | | containing 2 | | UBXDC1; |
| | | | | | | (UBXD2), | | FLJ23318; |
| | | | | | | mRNA. | | KIAA0242 |
| 143 | 192 | — | 610112 | NM_173607.3 | NM_173607 | chromosome | C14ORF24 | DKFZp686J1254; |
| | | | | | | 14 open | | FLJ38854 |
| | | | | | | reading frame | | |
| | | | | | | 24 (C14orf24), | | |
| | | | | | | transcript | | |
| | | | | | | variant 1, | | |
| | | | | | | mRNA. | | |

| | | P or N | | | | | |
|---|---|---|---|---|---|---|---|
| | | Predictor | | | | | |
| | | or | | | ABI Gene | | |
| RNA143 Index | Probe Sequence | HSK gene | Performance Rank | ABI Assay ID | Symbol | ABI Alias | ABI Gene Name |
| 1 | CAGCTGTG | N | 002N | Hs00240532_s1 | ADRB2 | ADRB2R, | adrenergic, |
| | AACATGGA | | | | | ADRBR, B2AR, | beta-2-, |
| | CTCTTCCCC | | | | | BAR, | receptor, |
| | CACTCCTCT | | | | | BETA2AR | surface |
| | TATTTGCTC | | | | | | |
| | ACACGGG | | | | | | |
| 2 | CCAGCCCA | P | 002P | Hs00270173_s1 | NAP1L3 | MB20, MGC26312, NPL3, | nucleosome |
| | TAAGACTAA | | | | | RP1- | assembly |
| | GGGTTTAAA | | | | | 32F7.3 | protein 1-like 3 |
| | TCTGCTTGC | | | | | | |
| | ACTAGCTGT | | | | | | |
| | GCCTTC | | | | | | |
| 3 | GTTCCTTTT | N | 003N | Hs00967385_g1 | SERBP1 | CGI- | SERPINE1 |
| | GCTGCCCA | | | | | 55, CHD3IP, | mRNA |
| | TTTGGGAG | | | | | DKFZp564M2423, | binding |
| | TATGTGGC | | | | | FLJ90489, | protein 1 |
| | AATTCCTAG | | | | | HABP4L, | |
| | TGCTCTTG | | | | | PAI- | |
| | | | | | | RBP1, PAIRBP1 | |
| 4 | GCCTTTTTA | P | 003P | Hs00211126_m1 | CSAD | CSD, FLJ44987, | cysteine |
| | GGCCACAG | | | | | FLJ45500, | sulfinic acid |
| | TGACCTGC | | | | | MGC119354, | decarboxylase |
| | GCAATGTTT | | | | | MGC119355, | |
| | ATATGCTTT | | | | | MGC119357, | |
| | GACCTAC | | | | | PCAP | |
| 5 | TCCTTAGTC | N | 005N | Hs00233566_m1 | CD79A | IGA, MB-1 | CD79a |
| | ATATTCCCC | | | | | | molecule, |
| | CAGTGGGG | | | | | | immunoglobulin- |
| | GGTGGGAG | | | | | | associated |
| | GGTAACCT | | | | | | alpha |
| | CACTCTTC | | | | | | |
| 6 | CCTGCGTC | P | 005P | Hs02340038_g1 | RPL37 | DKFZp686G1699, | ribosomal |
| | ACAGGGAA | | | | | MGC99572 | protein L37 |
| | GCAACCTA | | | | | | |
| | CAGAGAAG | | | | | | |
| | CAGCAGCT | | | | | | |
| | CCCCAAGA | | | | | | |
| | GA | | | | | | |
| 7 | ATCCCAGC | P | 006P | Hs00959834_m1 | XRCC1 | RCC | X-ray repair |
| | TTTGAGGA | | | | | | complementing |
| | GGCCCTGA | | | | | | defective |
| | TGGACAAC | | | | | | repair in |
| | CCCTCCCT | | | | | | Chinese |
| | GGCATTCG | | | | | | hamster cells 1 |
| | TT | | | | | | |
| 8 | TAGTGGCT | N | 007N | Hs00227769_m1 | POF1B | FLJ22792, | premature |
| | GGGAAAAG | | | | | POF, POF2B, | ovarian |
| | GGGTGTGC | | | | | RP1- | failure, 1B |
| | GAGGGGAA | | | | | 75N13.2 | |
| | CTGGGGAT | | | | | | |
| | GCTTAATGTG | | | | | | |
| 9 | CACCTCGG | P | 007P | Hs00255244_m1 | RABL2B | FLJ93981, | RAB, member |
| | GGACAATT | | | | | FLJ98216, | of RAS |
| | CCTTGGGC | | | | | FLJ78724, | oncogene |
| | TTCTCCTGA | | | | | MGC117180, | family-like |
| | GGTAATGAT | | | | | RP11- | 2B, RAB, |
| | TTACCCCC | | | | | 395L14.2 | member of |
| | | | | | | | RAS |
| | | | | | | | oncogene |
| | | | | | | | family-like 2A |
| 10 | AGGAGCCT | N | 009N | Hs01033772_g1 | NCOA4 | ARA70, DKFZp762E1112, | nuclear |
| | TTCCAGTTA | | | | | ELE1, | receptor |
| | TCTTGAGTT | | | | | PTC3, | coactivator 4 |
| | GCAGCTCT | | | | | RFG, RP11- | |
| | GTAGTTTCT | | | | | 481A12.4 | |
| | TGAGGCC | | | | | | |
| 11 | GCCACGCT | N | 011N | Hs00292978_m1 | KIAA1949 | DAAP- | KIAA1949 |
| | TACTTGCTG | | | | | 285E11.2, | |
| | TGTCTGCG | | | | | HKMT1098 | |
| | TGGAATTCT | | | | | | |
| | CTCCTCTGT | | | | | | |
| | CCCCTCC | | | | | | |
| 12 | GACCCAAA | P | 011P | Hs00270322_m1 | TAF1 | BA2R, CCG1, | TAF1 RNA |
| | CAACCCCG | | | | | CCGS, | polymerase II, |
| | CATGCTTCA | | | | | DYT3, DYT3/ | TATA box |
| | GGAGAACA | | | | | TAF1, | binding |
| | CAAGGATG | | | | | KAT4, N- | protein (TBP)- |
| | GACATGGAA | | | | | TAF1, NSCL2, | associated |
| | | | | | | OF, P250, | factor, |
| | | | | | | TAF2A, | 250 kDa |
| | | | | | | TAFII250, | |
| | | | | | | XDP | |
| 13 | GTACCAGA | P | 012P | Hs00176278_m1 | DGKA | DAGK, DAGK1, | diacylglycerol |
| | CCTAAGTG | | | | | DGK- | kinase, alpha |
| | ACAAGAGA | | | | | alpha, MGC12821, MGC42356 | 80 kDa |
| | CTGGAAGT | | | | | | |
| | GGTTGGGC | | | | | | |
| | TGGAGGGT | | | | | | |
| | GC | | | | | | |
| 14 | AGGATGCA | N | 013N | Hs00377534_m1 | TTC21A | DKFZp686P18239, | tetratricopeptide |
| | GTCACCAA | | | | | MGC156293, | repeat |
| | CTACAAACT | | | | | MGC70523, | domain 21A |
| | GGCCTGGA | | | | | STI2 | |
| | AGTACAGT | | | | | | |
| | CATCACGCC | | | | | | |
| 15 | GCTCTATCC | N | 014N | Hs02825719_s1 | GPR119 | GPCR2, MGC119957, | G protein- |
| | TGGACCCC | | | | | RP1- | coupled |
| | CTTCCTTAT | | | | | 20I3.4 | receptor 119 |
| | CACTGGCA | | | | | | |
| | TTGTGCAG | | | | | | |
| | GTGGCCTG | | | | | | |
| 16 | CTGTACTTC | N | 015N | Hs00221046_m1 | SENP7 | KIAA1707, | SUMO1/sentrin |
| | CACGTGAC | | | | | MGC157730 | specific |
| | TGGGTGCT | | | | | | peptidase 7 |
| | GAGGGGAG | | | | | | |
| | TTAAAGCCT | | | | | | |
| | CCCTGGTG | | | | | | |
| 17 | TCCAAGAG | P | 015P | Hs00357776_g1 | GRK6 | FLJ32135, | G protein- |
| | CTGAATGTC | | | | | GPRK6 | coupled |
| | TTTGGGCT | | | | | | receptor |
| | GGATGGCT | | | | | | kinase 6 |
| | CAGTTCCC | | | | | | |
| | CCAGACCTG | | | | | | |
| 18 | ACCTTGGC | N | 016N | Hs01018047_m1 | ABHD12 | ABHD12A; | abhydrolase |
| | TACAGGCA | | | | | BEM46L2, | domain |
| | CAAATACAT | | | | | C20ORF22, | containing 12 |
| | TTACAAGAG | | | | | DKFZp434P106, | |
| | CCCTGAGC | | | | | RP5- | |
| | TGCCACGG | | | | | 965G21.2, | |
| | | | | | | dJ965G21.2 | |
| 19 | GCATGGGG | P | 016P | Hs00757030_m1 | PRPF3 | HPRP3, HPRP3P, PRP3, | PRP3 pre- |
| | CTGAACACT | | | | | Prp3p, | mRNA |
| | ACTGGGAC | | | | | RP18 | processing |
| | CTTGCGCT | | | | | | factor 3 |
| | GAGTGAAT | | | | | | homolog (S. cerevisiae) |
| | CTGTGTTAG | | | | | | |
| 20 | GTTGTTTCA | N | 017N | Hs01850784_s1 | FOXG1 | BF1, BF2, | forkhead box |
| | GTTGGCAA | | | | | FHKL3, FKH2, | G1 |
| | CACTGCCC | | | | | FKHL1, | |
| | ATTCAATTG | | | | | FKHL2, | |
| | AATCAGAA | | | | | FKHL3, FKHL4, | |
| | GGGGACAA | | | | | FOXG1A, | |
| | | | | | | FOXG1B, | |
| | | | | | | FOXG1C, | |
| | | | | | | HBF- | |
| | | | | | | 1, HBF- | |
| | | | | | | 2, HBF- | |
| | | | | | | 3, HBF- | |
| | | | | | | G2, HBF2, | |
| | | | | | | HFK1, HFK2, | |
| | | | | | | HFK3, | |
| | | | | | | KHL2, QIN | |
| 21 | GGGAAGAT | P | 017P | Hs00538167_m1 | IL12RB1 | CD212, IL- | interleukin 12 |
| | GCCCTATCT | | | | | 12R- | receptor, beta 1 |
| | CTCGGGTG | | | | | BETA1, IL12RB, | |
| | CTGCCTAC | | | | | MGC34454 | |
| | AACGTGGC | | | | | | |
| | TGTCATCTC | | | | | | |
| 22 | GATAGGCC | P | 018P | Hs00262564_m1 | ZFYVE19 | FLJ14840, | zinc finger, |
| | CCTTCCTGA | | | | | MPFYVE | FYVE domain |
| | GCCTTGGT | | | | | | containing 19 |
| | GTCCCTGG | | | | | | |
| | AATGAGGA | | | | | | |
| | AAGATTCTC | | | | | | |
| 23 | TCCCACTTT | N | 019N | Hs01035151_m1 | CRTAP | CASP, LEPREL3 | cartilage |
| | AGGGTGGC | | | | | | associated |
| | AGCCAGTA | | | | | | protein |
| | GGCCAAAC | | | | | | |
| | TCCAAAGA | | | | | | |
| | CCGTTGCTG | | | | | | |
| 24 | CCACAGGT | N | 020N | Hs00198882_m1 | LAPTM5 | CLAST6, | lysosomal |
| | TAGTTCAGT | | | | | FLJ61683, | protein |
| | CAAAGCAG | | | | | FLJ97251, | transmembrane 5 |
| | GCAACCCC | | | | | MGC125860, | |
| | CTTGTGGG | | | | | MGC125861, | |
| | CACTGACCC | | | | | RP5- | |
| | | | | | | 1166H10.3 | |
| 25 | GATCTTGG | N | 021N | Hs00243566_m1 | B4GALT2 | B4Gal- | UDP- |
| | GGTTGGCC | | | | | T2, B4Gal- | Gal:betaGlcNAc |
| | TTTGCATGG | | | | | T3, beta4Gal- | beta 1,4- |
| | GAGGCAGG | | | | | T2 | galactosyltransferase, |
| | TGGGGCTT | | | | | | polypeptide 2 |
| | GGATCAGTA | | | | | | |
| 26 | CTCAGCTG | P | 021P | Hs00195582_m1 | S100A2 | CAN19, MGC111539, | S100 calcium |
| | GAGTGCTG | | | | | RP11- | binding |
| | GGAGATGA | | | | | 49N14.8, | protein A2 |
| | GGGCCTCC | | | | | S100L | |
| | TGGATCCT | | | | | | |
| | GCTCCCTT | | | | | | |
| | CT | | | | | | |
| 27 | GGCCCTTC | P | 022P | Hs00159587_m1 | NDUFC1 | KFYI, MGC117464, | NADH |
| | AGTGCGAT | | | | | MGC126847, | dehydrogenase |
| | CAAAGTTCT | | | | | MGC138266 | (ubiquinone) |
| | ACGTGCGA | | | | | | 1, |
| | GAGCCGCC | | | | | | subcomplex |
| | GAATGCCAA | | | | | | unknown, 1, |
| | | | | | | | 6 kDa |
| 28 | TGCACAAAA | N | 023N | Hs00609198_m1 | GCH1 | DYT14, DYT5, | GTP |
| | CCACTGCC | | | | | DYT5a, | cyclohydrolase 1 |
| | AGATAACCA | | | | | GCH, | |
| | GAGGGGCC | | | | | GTP-CH- | |
| | TGGGAAGG | | | | | 1, GTPCH1, | |
| | GAGAAGAA | | | | | HPABH4B | |
| 29 | GTCCACTA | P | 023P | Hs00197131_m1 | PIBF1 | C13ORF24, | progesterone |
| | CGAGGTAC | | | | | KIAA1008, | immunomodulatory |
| | TTCAAAAGC | | | | | PIBF, RP11- | binding |
| | CCAGTAAT | | | | | 505F3.1 | factor 1 |
| | GGTGGTCA | | | | | | |
| | GATACCATG | | | | | | |
| 30 | TTCCACCAC | P | 024P | Hs00255552_m1 | TMEM8B | C9ORF127, | transmembrane |
| | GTTCTCCG | | | | | MGC120460, | protein 8B |
| | AGGGTTTG | | | | | NAG- | |
| | GGAATGTC | | | | | 5, NGX6, RP11- | |
| | TGTGCCTTC | | | | | 112J3.10, | |
| | ACTGTGTC | | | | | RP11- | |
| | | | | | | 112J3.10- | |
| | | | | | | 001 | |
| 31 | CTGGCTCC | P | 026P | Hs00179978_m1 | EPS15 | AF- | epidermal |
| | AGGGCCTG | | | | | 1P, AF1P, | growth factor |
| | TGCTTGAAA | | | | | MLLT5 | receptor |
| | AGGACAGA | | | | | | pathway |
| | TAAGTATTG | | | | | | substrate 15 |
| | CCCAGAGC | | | | | | |
| 32 | CAGGTTGT | N | 027N | Hs00195343_m1 | SMNDC1 | SMNR, SPF30 | survival motor |
| | CTGCATTTG | | | | | | neuron |
| | TTGGTGTAA | | | | | | domain |
| | GTGAACAT | | | | | | containing 1 |
| | CATCACAGT | | | | | | |
| | TATCCTG | | | | | | |
| 33 | CCCTCTGT | P | 027P | Hs00223885_m1 | MPP5 | FLJ12615; | membrane |
| | GGTTCTGA | | | | | PALS1 | protein, |
| | CTGGAGAC | | | | | | palmitoylated |
| | CCCAGTGT | | | | | | 5 (MAGUK |
| | GGGGGAGG | | | | | | p55 subfamily |
| | TCTTACCATT | | | | | | member 5) |
| 34 | AATTGCTG | P | 028P | Hs00275076_m1 | HEMK1 | FLJ22320, | HemKmethyltransferase |
| | GAGGAAGA | | | | | HEMK, MTQ1 | family |
| | GCCATGAG | | | | | | member 1 |
| | CCGAGGAA | | | | | | |
| | TGCAGACA | | | | | | |
| | GCCTCTTCTC | | | | | | |
| 35 | GGCACTTG | N | 029N | Hs00893626_m1 | IL1RN | DIRA, ICIL- | interleukin 1 |
| | GAGACTTG | | | | | 1RA, IL- | receptor |
| | TATGAAAGA | | | | | 1RN, IL- | antagonist |
| | TGGCTGTG | | | | | 1ra, IL- | |
| | CCTCTGCC | | | | | 1ra3, IL1F3, | |
| | TGTCTCCCC | | | | | IL1RA, IRAP, | |
| | | | | | | MGC10430, MVCD4 | |
| 36 | GCAGCGAC | N | 031N | Hs00193519_m1 | MAF | MGC71685, | v- |
| | AACCCGTC | | | | | c-MAF | mafmusculoaponeuroticfibrosarcoma |
| | CTCTCCCG | | | | | | oncogene |
| | AGTTTTTCA | | | | | | homolog |
| | TAACTGAG | | | | | | (avian) |
| | CCCACTCGC | | | | | | |
| 37 | AGGGCAGG | N | 033N | Hs00376245_m1 | GSG1L | MGC18079, | GSG1-like |
| | CCCAAGGG | | | | | PRO19651, | |
| | AATGCACA | | | | | UNQ5831 | |
| | GGGCTGCA | | | | | | |
| | CAGAGTGA | | | | | | |
| | CTTTGGGA | | | | | | |
| | CA | | | | | | |
| 38 | TGTTTGAGT | P | 033P | Hs00212889_m1 | PPHLN1 | HSPC206, | periphilin 1 |
| | TGACTTCAC | | | | | HSPC232, | |
| | AGTCAGTTT | | | | | MGC48786 | |
| | GATCAGTAT | | | | | | |
| | GGTCCCCC | | | | | | |
| | ACCTGG | | | | | | |
| 39 | TGCTGCAA | P | 034P | Hs01122781_m1 | KIF3B | HH0048, KIAA0359 | kinesin family |
| | CTGGGGCG | | | | | | Member 3B |
| | TGGGCCGC | | | | | | |
| | TCTCTGCTT | | | | | | |
| | TTCCTGTCT | | | | | | |
| | GACTCTGA | | | | | | |
| 40 | GGGGGTGG | N | 035N | Hs00364814_m1 | IL22RA2 | CRF2- | interleukin 22 |
| | AGGAGAAT | | | | | 10, CRF2- | receptor, |
| | AAGAGGCA | | | | | S1, CRF2X, | alpha 2 |
| | GAGCAAGA | | | | | IL- | |
| | GCTAGAGA | | | | | 22BP, IL- | |
| | ATTGGTTTCC | | | | | 22RA2, MGC150509, | |
| | | | | | | MGC150510, | |
| | | | | | | UNQ5793/PRO19598/ | |
| | | | | | | PRO19822 | |
| 41 | CCTGGGCA | P | 035P | Hs00912503_m1 | PIGO | DKFZp434M222, | phosphatidylinositol |
| | TAGCTTTGG | | | | | FLJ00135, MGC20536, | glycan |
| | TGATGAGA | | | | | MGC3079, | anchor |
| | GTGGATGG | | | | | RP11- | biosynthesis, |
| | TGCTGTGA | | | | | 182N22.4, | class O |
| | GCTCCTGGT | | | | | UNQ632/ | |
| | | | | | | PRO1249 | |
| 42 | CAGACAAA | N | 036N | Hs00244603_m1 | SERPINB9 | CAP- | serpin |
| | CTGTTTTCC | | | | | CAP3, PI9 | peptidase |
| | ACAGCAGT | | | | | | inhibitor, clade |
| | TGAACCATT | | | | | | B (ovalbumin), |
| | CCACATTCC | | | | | | member 9 |
| | CACCAGC | | | | | | |
| 43 | GTAGCCAA | P | 036P | Hs01053640_m1 | TXK | BTKL, MGC22473, PSCTK5, PTK4, | TXK tyrosine |
| | AGCTCACC | | | | | RLK, | kinase |
| | TTTGAACAG | | | | | TKL | |
| | ATCCCGGT | | | | | | |
| | GACATTCTA | | | | | | |
| | TTTCCAGG | | | | | | |
| 44 | GGCAGTTT | N | 037N | Hs00368207_m1 | PREX1 | KIAA1415, | phosphatidylinositol- |
| | GTCCCCCC | | | | | P-REX1 | 3,4,5- |
| | AGCTTCGG | | | | | | trisphosphate- |
| | TATGCCTTC | | | | | | dependent |
| | AGGGAAAG | | | | | | Rac exchange |
| | GTCACAGCT | | | | | | factor 1 |
| 45 | ACGTGCTC | N | 038N | Hs00216128_m1 | NSUN5 | FLJ10267, | NOP2/Sun |
| | CCTCTGCC | | | | | MGC986, | domain family, |
| | AGGAGGAG | | | | | NOL1, NOL1R, | member 5 |
| | AATGAAGA | | | | | NSUN5A, | |
| | CGTGGTGC | | | | | WBSCR20, | |
| | GAGATGCG | | | | | WBSCR20A, | |
| | CT | | | | | p120 | |
| 46 | GACAGATG | P | 038P | Hs00204112_m1 | MRPL42 | HSPC204, | mitochondrial |
| | ATGCGGAG | | | | | MRP- | ribosomal |
| | GTTCCTGG | | | | | L31, MRPL31, | protein L42 |
| | GGGAATCA | | | | | MRPS32, | |
| | AAGAGAAAT | | | | | PTD007, | |
| | GTGCCTCAT | | | | | RPML31 | |
| 47 | TCAGACCG | N | 039N | Hs00384853_m1 | SIPA1L2 | FLJ23126, | signal-induced |
| | AGAAGCAG | | | | | FLJ23632, | proliferation- |
| | GGTGAGAG | | | | | KIAA1389, | associated 1 |
| | ATTCTAACG | | | | | SPAL2 | like 2 |
| | ACTGGATG | | | | | | |
| | CTGCTAGTA | | | | | | |
| 48 | ATCAGGCC | P | 039P | Hs00234934_m1 | CACNA1H | CACNA1HB, | calcium |
| | TCCCCTACA | | | | | Cav3.2, | channel, |
| | TCTGGGGG | | | | | ECA6, EIG6, | voltage- |
| | CGTTGGCC | | | | | FLJ90484 | dependent, T |
| | GCGAGATT | | | | | | type, alpha 1H |
| | CCCATTGAC | | | | | | subunit |
| 49 | TGTACTTGG | N | 040N | Hs00829622_s1 | ZFAND5 | RP11- | zinc finger, |
| | GTGTAGGA | | | | | 63P12.8, ZA20D2, | AN1-type |
| | CTCTAGTGT | | | | | ZFAND5A, ZNF216 | domain 5 |
| | TCTTGGGT | | | | | | |
| | GTATTGCAT | | | | | | |
| | GGGCTGC | | | | | | |
| 50 | AGAGCCTT | P | 040P | Hs00183813_m1 | PDCD6IP | AIP1, Alix, | programmed |
| | GTGTCCCT | | | | | DRIP4, HP95, | cell death 6 |
| | AAAGTTCTG | | | | | MGC17003 | interacting |
| | TCCCAGTC | | | | | | protein |
| | AGCAGTCTT | | | | | | |
| | TATAGTCC | | | | | | |
| 51 | AGGCCTGG | N | 041N | Hs00379444_m1 | NCDN | KIAA0607 | neurochondrin |
| | TGGGGGGT | | | | | | |
| | GGGGAAAC | | | | | | |
| | CTCCTTCCA | | | | | | |
| | CCTGAGCT | | | | | | |
| | TGCTTGAAG | | | | | | |
| 52 | GAGCTGGG | P | 041P | Hs00762282_s1 | VTI1B | VTI1, VTI1- | vesicle |
| | GGAACAAC | | | | | LIKE, VTI1L, | transport |
| | GAGACCAG | | | | | VTI2 | through |
| | TTAGAACGT | | | | | | interaction |
| | ACCAAGAG | | | | | | with t- |
| | TAGACTGGT | | | | | | SNAREs |
| | | | | | | | homolog 1B |
| | | | | | | | (yeast) |
| 53 | CGGTGTGT | P | 042P | Hs00757279_mH | TATDN1 | CDA11, FLJ43280 | TatDDNase |
| | AGGGGGAG | | | | | | domain |
| | TGGTGCATT | | | | | | containing 1 |
| | CATTTGATG | | | | | | |
| | GTACCAAG | | | | | | |
| | GAAGCAGC | | | | | | |
| 54 | AGAGGAGA | N | 043N | Hs00193731_m1 | CCDC6 | D10S170, | coiled-coil |
| | GCCAAGCG | | | | | FLJ32286, | domain |
| | CTAGCATG | | | | | H4, PTC, | containing 6 |
| | CCTTTTGCC | | | | | TPC, TST1 | |
| | TCTGCATAT | | | | | | |
| | CTGTGCAC | | | | | | |
| 55 | GGAGCTGC | P | 043P | Hs00260456_m1 | COQ5 | MGC104303, | coenzyme Q5 |
| | TTCAGTCCA | | | | | MGC4767 | homolog, |
| | TCTCCCAG | | | | | | methyltransferase |
| | AGGCATTT | | | | | | (S. cerevisiae) |
| | GGTCTGTAT | | | | | | |
| | CTTTGCTC | | | | | | |
| 56 | ACTGCCAG | N | 044N | Hs00939664_m1 | FOXN2 | HTLF | forkhead box |
| | TAGATGAC | | | | | | N2 |
| | CAGTCACA | | | | | | |
| | AGTGAACC | | | | | | |
| | ACTTCTCAG | | | | | | |
| | TTGCCAATC | | | | | | |
| 57 | ACCGCACG | P | 044P | Hs01126016_m1 | UTRN | DMDL, DRP, | utrophin |
| | ACACCAGC | | | | | DRP1, FLJ23678, | |
| | ACGGATCT | | | | | RP11- | |
| | CACGGAGG | | | | | 352E13.1 | |
| | TCATGGAG | | | | | | |
| | CAGATTCACA | | | | | | |
| 58 | GGTGGGTG | N | 045N | Hs01115513_m1 | PPARG | CIMT1, GLM1, | peroxisome |
| | TGTAGTCGT | | | | | NR1C3, | proliferator- |
| | GGTACTTTA | | | | | PPARG1, | activated |
| | CGCCTCGG | | | | | PPARG2, | receptor |
| | TGTTTAGG | | | | | PPARg | gamma |
| | GAGGAGCC | | | | | amma | |
| 59 | GTCTCGGT | P | 045P | Hs00219487_m1 | CCDC76 | FLJ10287, | coiled-coil |
| | TCAAATTCC | | | | | FLJ11219, | domain |
| | AAACCTACC | | | | | RP11- | containing 76 |
| | ATCTTCAGT | | | | | 305E17.1 | |
| | TGTGCGAC | | | | | | |
| | CTTGGGC | | | | | | |
| 60 | AGGCCTCT | N | 046N | Hs00212858_m1 | ANAPC11 | APC11, Apc11p, | anaphase |
| | GGGTGCCT | | | | | HSPC214, MGC882 | promoting |
| | GTGTTCTC | | | | | | complex |
| | GGCATATA | | | | | | subunit 11 |
| | GATGTGGT | | | | | | |
| | CTCGGTGT | | | | | | |
| | GT | | | | | | |
| 61 | CACTAAACC | P | 046P | Hs00903035_g1 | EI24 | PIG8, TP53I8 | etoposide |
| | TGAACTTTT | | | | | | induced 2.4 |
| | CAACTCCG | | | | | | mRNA |
| | TTGGTGGT | | | | | | |
| | GGGAGGCA | | | | | | |
| | GCGGGCAG | | | | | | |
| 62 | GGTTGGGG | N | 047N | Hs00427977_m1 | ERH | DROER, FLJ27340 | enhancer of |
| | TGGGCTTG | | | | | | rudimentary |
| | GAACACAG | | | | | | homolog |
| | GTGTGTAC | | | | | | (Drosophila) |
| | AGCGTGCT | | | | | | |
| | GTAGTGGA | | | | | | |
| | AG | | | | | | |
| 63 | GGAGATTA | P | 047P | Hs00383486_m1 | CC2D1B | KIAA1836, | coiled-coil and |
| | GTGACTCA | | | | | RP11- | C2 domain |
| | CCTGCAGT | | | | | 155O18.2 | containing 1B |
| | TGGGAGCC | | | | | | |
| | AGCTACAA | | | | | | |
| | CCCAAATCAT | | | | | | |
| 64 | CTTCCAAG | N | 048N | Hs00295839_m1 | RCHY1 | ARNIP, CHIMP, | ring finger and |
| | GGCTAGGA | | | | | DKFZp586C1620, | CHY zinc |
| | TTACAGGC | | | | | PIRH2, | finger domain |
| | ATGAGCCA | | | | | PRO1996, | containing 1 |
| | CTGTGCTT | | | | | RNF199, | |
| | GGTCCAGA | | | | | ZNF363, | |
| | TG | | | | | hARNIP | |
| 65 | GGGCCATT | P | 048P | Hs00197392_m1 | TM9SF1 | HMP70, MP70 | transmembrane 9 |
| | AACTCAGC | | | | | | superfamily |
| | AGCCATCTT | | | | | | member 1 |
| | GTTGTATGC | | | | | | |
| | CCTGACCT | | | | | | |
| | GCTGCATC | | | | | | |
| 66 | AGGGCCAC | N | 049N | Hs01651150_m1 | ENTPD8 | GLSR2492, | ectonucleoside |
| | ATGCTGCC | | | | | NTPDase- | triphosphate |
| | TGCAAACA | | | | | 8, UNQ2492, | diphosphohydrolase 8 |
| | GGGCAAGA | | | | | UNQ2492/ | |
| | CCACGGAG | | | | | PRO5779 | |
| | GCACAGGG | | | | | | |
| | GT | | | | | | |
| 67 | CTGAAGGG | P | 049P | Hs00398565_m1 | C3orf15 | AAT1, AAT1alpha, DKFZp781A2221 | chromosome |
| | CCAGATGG | | | | | | 3 open |
| | TAACTACAT | | | | | | reading frame |
| | TAGGGTTT | | | | | | 15 |
| | GCGGGTCT | | | | | | |
| | GATGGTCGC | | | | | | |
| 68 | AGCTGAAA | N | 050N | Hs00381867_m1 | FGFR1OP2 | DKFZp564O1863, DKFZp586C1423, | FGFR1 |
| | GTGGGGGT | | | | | FLJ37569, HSPC123, | oncogene |
| | AAAGGTGG | | | | | HSPC123- | partner 2 |
| | AGTAATCTG | | | | | like, WIT3.0 | |
| | TGGATTTGT | | | | | | |
| | TTCTGTTG | | | | | | |
| 69 | GATCCTAG | P | 050P | Hs00274505_m1 | NCAPD2 | CAP- | non-SMC |
| | GAAGTCTG | | | | | D2, CNAP1, | condensin I |
| | TTCCTGTCC | | | | | KIAA0159, | complex, |
| | TCCCTGTG | | | | | hCAP- | subunit D2 |
| | CAGGGTAT | | | | | D2 | |
| | CCTGTAGGG | | | | | | |
| 70 | TGCCCAGG | N | 051N | Hs00152825_m1 | TLR5 | FLJ10052, | toll-like |
| | GCAGGTGC | | | | | MGC126430, | receptor 5 |
| | TTATCTGAC | | | | | MGC126431, RP11- | |
| | CTTAACAGT | | | | | 239E10.1, | |
| | GCTCTCATC | | | | | SLEB1, TIL3 | |
| | ATGGTGG | | | | | | |
| 71 | GAAAGGAG | N | 052N | Hs00542678_m1 | NFATC1 | MGC138448, | nuclear factor |
| | AGACGGAC | | | | | NF- | of activated T- |
| | ATCGGGAG | | | | | ATC, NFAT2, | cells, |
| | GAAGAACA | | | | | NFATc | cytoplasmic, |
| | CACGGGTA | | | | | | calcineurin- |
| | CGGCTGGT | | | | | | dependent 1 |
| | GT | | | | | | |
| 72 | GCTGTTGG | P | 052P | Hs00226305_m1 | ARHGAP10 | FLJ20896, | Rho GTPase |
| | TGCAAGGG | | | | | FLJ41791, | activating |
| | AGATGGTC | | | | | GRAF2, | protein 10 |
| | TCAAGTCA | | | | | PS- | |
| | GAGGGAAG | | | | | GAP, PSGAP | |
| | CAGAGACG | | | | | | |
| | CG | | | | | | |
| 73 | TGGCTCCC | N | 053N | Hs00369703_m1 | RPUSD1 | C16ORF40, | RNA |
| | ACACAGCC | | | | | MGC19600, | pseudouridylate |
| | ATGCATTGT | | | | | RLUCL | synthase |
| | CACTCTGC | | | | | | domain |
| | CTCCGGGA | | | | | | containing 1 |
| | CCCCAGCTT | | | | | | |
| 74 | CCCACATC | N | 054N | Hs00226971_m1 | MMRN2 | EMILIN3, | multimerin 2 |
| | AAGGGAGA | | | | | EndoGlyx- | |
| | GATCAGGT | | | | | 1, FLJ13465 | |
| | GGAGGTAA | | | | | | |
| | TTGGATCTT | | | | | | |
| | GGGGGCG | | | | | | |
| | GT | | | | | | |
| 75 | CTGTGGTG | P | 054P | Hs00212785_m1 | MPP6 | PALS2, VAM- | membrane |
| | GATGCAGG | | | | | 1, VAM1, p55T | protein, |
| | AATCACTAC | | | | | | palmitoylated |
| | CAAGCTTCT | | | | | | 6 (MAGUK |
| | GACCGACT | | | | | | p55 subfamily |
| | CTGACTTG | | | | | | member 6) |
| 76 | GCTTCTACA | N | 055N | Hs00185020_m1 | AQP3 | GIL | aquaporin 3 |
| | GGCTTTTG | | | | | | (Gill blood |
| | GGAAGTAG | | | | | | group) |
| | GGTGGATG | | | | | | |
| | TGGGTAGG | | | | | | |
| | GCTGGGAGG | | | | | | |
| 77 | GTTGAGGA | P | 055P | Hs00197140_m1 | COG5 | CDG2I, FLJ41732, FLJ44289, | component of |
| | ACCACTGG | | | | | GOLTC1, | oligomericgolgi |
| | CACATCCC | | | | | GTC90 | complex 5 |
| | AAGCTAAG | | | | | | |
| | ATACAAGGT | | | | | | |
| | TAAATGGCC | | | | | | |
| 78 | AGAGTCGC | N | 056N | Hs00368084_m1 | RARS2 | ArgRS, DALRD2, | arginyl- |
| | GGGGACAC | | | | | MGC14993, MGC23778, | tRNAsynthetase |
| | AGGAGTCT | | | | | PCH6, PRO1992, RARSL, | 2, |
| | TCCTACAGT | | | | | RP3- | mitochondrial |
| | ACACACAC | | | | | 382I10.6, | |
| | GCCCGCCTC | | | | | dJ382I10.6 | |
| 79 | CGTGGGAC | P | 056P | Hs01582977_gH | MT1E | MT1, MTD | metallothionein |
| | ACAAACCC | | | | | | 1E |
| | CAACTGTAC | | | | | | |
| | CCCCTATG | | | | | | |
| | GTTTCAGAA | | | | | | |
| | CAGAGCTG | | | | | | |
| 80 | GCTGAGCG | N | 057N | Hs00217534_m1 | WDR41 | FLJ10904, | WD repeat |
| | CAACTGCC | | | | | MSTP048 | domain 41 |
| | CCATCTGA | | | | | | |
| | CCACTGAC | | | | | | |
| | TCAAATACG | | | | | | |
| | AACTGCTTG | | | | | | |
| 81 | CAGAAGTG | N | 058N | Hs00872692_m1 | NRK | DKFZp686A17109, | Nik related |
| | TGGAGGGG | | | | | FLJ16788, | kinase |
| | GGCTCCTG | | | | | MGC131849, | |
| | ACTAGACAA | | | | | NESK, | |
| | TTTCCCTAG | | | | | RP1- | |
| | CCCTTGTG | | | | | 82J11.1 | |
| 82 | TCCCTTCAA | P | 058P | Hs00275795_m1 | SPG7 | CAR, CMAR, | spastic |
| | CGTAGTCAT | | | | | FLJ37308, | paraplegia 7 |
| | CCCCTGGT | | | | | MGC126331, MGC126332, | (pure and |
| | GGTGGAAG | | | | | PGN, SPG5C | complicated |
| | CAAGACGA | | | | | | autosomal |
| | CGGCCCCT | | | | | | recessive) |
| 83 | CTGCAGGT | N | 060N | Hs00165902_m1 | TCN2 | D22S676, | transcobalamin |
| | CTCCCATG | | | | | D22S750, | II |
| | AAGGCCAC | | | | | II, TC, TCII, | |
| | CCCATGGT | | | | | TC- | |
| | CTGATGGG | | | | | 2, TC2, TCII | |
| | CATGAAGC | | | | | | |
| | AT | | | | | | |
| 84 | GTCGTTCCT | N | 061N | Hs00203383_m1 | C7orf68 | FLJ21076, | chromosome |
| | CCAACATA | | | | | HIG- | 7 open |
| | GTGTGTATT | | | | | 2, HIG2, MGC138388 | reading frame |
| | GGTCTGAA | | | | | | 68 |
| | GGGGGTGG | | | | | | |
| | TGGGATGC | | | | | | |
| 85 | AGTACCTTG | N | 062N | Hs00163869_m1 | CA2 | CA- | carbonic |
| | ACTTTGTTC | | | | | II, CAII, Car2 | anhydrase II |
| | ACAGCATG | | | | | | |
| | TAGGGTGA | | | | | | |
| | TGAGCACT | | | | | | |
| | CACAATTG | | | | | | |
| 86 | GGCTTGGT | P | 062P | Hs00535769_m1 | C8orf33 | FLJ20989 | chromosome |
| | CTAGCAGT | | | | | | 8 open |
| | AACACCAG | | | | | | reading frame |
| | TGTCTGGG | | | | | | 33 |
| | AAGATGCC | | | | | | |
| | TGTTGCAAAG | | | | | | |
| 87 | TCTGTAGC | N | 063N | Hs00610137_m1 | STAM | DKFZp686J2352, STAM1 | signal |
| | CTCTGCATA | | | | | | transducing |
| | CTACTGGC | | | | | | adaptor |
| | TGTCATCAC | | | | | | molecule |
| | ACCAGCGT | | | | | | (SH3 domain |
| | ACAGTAGC | | | | | | and ITAM |
| | | | | | | | motif) 1 |
| 88 | GAAGAATG | P | 063P | Hs00224208_m1 | SMYD3 | FLJ21080, | SET and |
| | CGACGCCA | | | | | KMT3E, | MYND |
| | ACATCAGA | | | | | MGC104324, | domain |
| | GCATCCTAA | | | | | ZMYND1, | containing 3 |
| | GGGAACGC | | | | | ZNFN3A1, | |
| | AGTCAGAGG | | | | | bA74P14.1 | |
| 89 | CGCCCACC | N | 064N | Hs00608163_m1 | SEC14L1 | DKFZp686C06176, | SEC14-like 1 |
| | CAGCGGCG | | | | | PRELID4A, | (S. cerevisiae) |
| | ACATTGTAC | | | | | SEC14L | |
| | AGACTCCT | | | | | | |
| | CTCACCTCT | | | | | | |
| | AGATAGCA | | | | | | |
| 90 | ATGTCCTTG | P | 064P | Hs00265266_g1 | GSTM2 | GST4, GSTM, | glutathione S- |
| | AGAGAAAC | | | | | GSTM2- | transferase |
| | CAAGTATTT | | | | | 2, GTHMUS, | mu 2 (muscle) |
| | GAGCCCAG | | | | | MGC117303 | |
| | CTGCCTGG | | | | | | |
| | ATGCCTTC | | | | | | |
| 91 | GTGTGTTTT | N | 065N | Hs00698292_m1 | FADS6 | FP18279 | fatty acid |
| | GTCGGGAG | | | | | | desaturase |
| | GGAACTCC | | | | | | domain family, |
| | AGGGGAAG | | | | | | member 6 |
| | TGAGGGGA | | | | | | |
| | GAAGGTTCC | | | | | | |
| 92 | CGGCTGCA | P | 065P | Hs00853882_g1 | SNRPC | FLJ20302, | small nuclear |
| | TTTCAACAA | | | | | RP3- | ribonucleoprotein |
| | GGAAAGAT | | | | | 375P9.1, | polypeptide C |
| | ACCTCCTAC | | | | | U1C, Yhc1 | |
| | TCCATTCTC | | | | | | |
| | TGCTCCT | | | | | | |
| 93 | CCAGTATCA | N | 066N | Hs01040835_m1 | GINS1 | KIAA0186, | GINS |
| | CCACTTTG | | | | | PSF1, RP4- | complex |
| | GAAGGGGA | | | | | 691N24.2 | subunit 1 |
| | CAGTGAAAT | | | | | | (Psf1 |
| | TGGGGCTA | | | | | | homolog) |
| | GAGAAGGA | | | | | | |
| 94 | CCTTTTGAG | N | 067N | Hs00363121_m1 | POLR3K | C11, C11- | polymerase |
| | GTGAAGAG | | | | | RNP3, My010, | (RNA) III |
| | CCAGGGGG | | | | | RPC10, | (DNA |
| | TCAGGAAAT | | | | | RPC11, | directed) |
| | ATGGCCTAT | | | | | RPC12.5, | polypeptide K, |
| | CTGCCAG | | | | | hRPC11 | 12.3 kDa |
| 95 | CCCAGCCC | P | 067P | Hs00208576_m1 | RNF44 | KIAA1100 | ring finger |
| | TGGCTGGG | | | | | | protein 44 |
| | CCCAGCGC | | | | | | |
| | CTGTGTTCT | | | | | | |
| | GTGTTAGAA | | | | | | |
| | AGGTTTTA | | | | | | |
| 96 | AACTGGGC | N | 068N | Hs00362067_m1 | COX5A | COX, COX- | cytochrome c |
| | CTTGACAAA | | | | | VA, VA | oxidase |
| | GTGTAAAC | | | | | | subunit Va |
| | CGCATGGA | | | | | | |
| | TGGGCTTC | | | | | | |
| | CCCAAGGAT | | | | | | |
| 97 | GGCAATTTT | P | 068P | Hs00536084_m1 | PHAX | FLJ13193, | phosphorylated |
| | AAGGATAAA | | | | | RNUXA | adaptor for |
| | AACTAACAT | | | | | | RNA export |
| | TGGCCAGG | | | | | | |
| | CACGGTGG | | | | | | |
| | CTCACGC | | | | | | |
| 98 | TCTGTACAT | P | 069P | Hs00608563_m1 | HEATR3 | FLJ20718 | HEAT repeat |
| | TCTGTAAAA | | | | | | containing 3 |
| | ACTTCAAAA | | | | | | |
| | CCTGGCCA | | | | | | |
| | GGCATGGT | | | | | | |
| | GGCTCAC | | | | | | |
| 99 | GCCACCTC | N | 070N | Hs00260900_m1 | C5orf32 | ORF1- | chromosome |
| | TGACAGGT | | | | | FL49 | 5 open |
| | GTGCCTGC | | | | | | reading frame |
| | CCCCATCT | | | | | | 32 |
| | CTTCTGATT | | | | | | |
| | GCTGTTAAC | | | | | | |
| 100 | TCTGGACG | P | 070P | Hs00156055_m1 | BCL7B | 0 | B-cell |
| | GAGCTGCT | | | | | | CLL/lymphoma |
| | GGCAGCTT | | | | | | 7B |
| | CTGCGAGA | | | | | | |
| | AGAGAGAG | | | | | | |
| | ATGTGGAA | | | | | | |
| | GG | | | | | | |
| 101 | ATGATCCA | N | 071N | Hs00246261_m1 | HBXIP | MGC71071, | hepatitis B |
| | GAAACACG | | | | | XIP | virus × |
| | ATGGCATC | | | | | | interacting |
| | ACGGTGGC | | | | | | protein |
| | AGTGCACA | | | | | | |
| | AAATGGCC | | | | | | |
| | TC | | | | | | |
| 102 | TGCAACTG | P | 071P | Hs00217433_m1 | YY1AP1 | FLJ10875, | YY1 |
| | GGGCTCTT | | | | | FLJ13914, | associated |
| | GAGCAGCT | | | | | HCCA1, | protein 1 |
| | TGCTTTAGC | | | | | HCCA2, RP11- | |
| | CTGCTCCC | | | | | 243J18.1, | |
| | ACTCTGTGG | | | | | YAP, YY1AP | |
| 103 | CTTGCCGC | N | 072N | Hs00170832_m1 | CD226 | DNAM- | CD226 |
| | CATCCCAG | | | | | 1, DNAM1, | molecule |
| | GTCTAGCC | | | | | PTA1, TLiSA1 | |
| | TTAGGAGC | | | | | | |
| | AAATGTAGT | | | | | | |
| | AGATAGTCG | | | | | | |
| 104 | AGCCAGGG | P | 072P | Hs01075391_m1 | ZNF641 | DKFZp667D1012, FLJ31295 | zinc finger |
| | GGGCCAGA | | | | | | protein 641 |
| | CCTTGTTCA | | | | | | |
| | TGTGTGGG | | | | | | |
| | TCTGTCTTC | | | | | | |
| | CTTATGCC | | | | | | |
| 105 | TTGAAGATT | N | 073N | Hs00204415_m1 | HOXC11 | HOX3H, MGC4906 | homeobox |
| | GGGGTGGT | | | | | | C11 |
| | GGAGGCAG | | | | | | |
| | TAGGGAGA | | | | | | |
| | TGGGATTG | | | | | | |
| | GGCACCTCC | | | | | | |
| 106 | CCATGGCA | P | 073P | Hs00971557_m1 | KLF12 | AP- | Kruppel-like |
| | AAGCACAA | | | | | 2rep, AP2REP, | factor 12 |
| | ATGGACCC | | | | | HSPC122 | |
| | CCGAGGCC | | | | | | |
| | TATCTCCCA | | | | | | |
| | GACAAAGTA | | | | | | |
| 107 | ACCTCAAG | N | 074N | Hs00225908_m1 | C16orf53 | FLJ22459, | chromosome |
| | CTCCCAAA | | | | | GAS, MGC4606, | 16 open |
| | CAGCACGT | | | | | PA1 | reading frame |
| | TGCGGGAA | | | | | | 53 |
| | AGAGGAAG | | | | | | |
| | AGAGAGTG | | | | | | |
| | TG | | | | | | |
| 108 | AGCTGTGT | P | 074P | Hs00248344_m1 | SARM1 | FLJ36296, | sterile alpha |
| | GACCGGGA | | | | | KIAA0524, | and TIR motif |
| | GTAGTCACT | | | | | SAMD2, | containing 1 |
| | TAACCTATG | | | | | SARM | |
| | TCTCCCCTT | | | | | | |
| | CCTCACC | | | | | | |
| 109 | GAGGTATTT | N | 075N | Hs00905983_m1 | N4BP2 | B3BP, FLJ10680, | NEDD4 |
| | AAAGTGCTT | | | | | KIAA1413 | binding |
| | TGAGACCT | | | | | | protein 2 |
| | GATTCATGC | | | | | | |
| | CCCCCAAA | | | | | | |
| | GGGTGGT | | | | | | |
| 110 | ATGTATGGA | P | 075P | Hs01390827_g1 | LYRM5 | 0 | LYR motif |
| | GTCATTACT | | | | | | containing 5 |
| | TCTGACCTT | | | | | | |
| | GAAATAGC | | | | | | |
| | CTGCTGGT | | | | | | |
| | GACTGGC | | | | | | |
| 111 | CCAGGCTT | N | 076N | Hs00541038_m1 | SHMT1 | CSHMT, | serine |
| | TCCTGCTC | | | | | MGC15229, | hydroxymethyltransferase 1 |
| | CACCTGAG | | | | | MGC24556, | (soluble) |
| | ATAACCAAC | | | | | SHMT | |
| | TCCCTCCC | | | | | | |
| | GTAATCAGG | | | | | | |
| 112 | CTTCTCGCT | P | 076P | Hs00823168_g1 | MT1H | MGC70702, | metallothionein |
| | TGGGAACT | | | | | MT1 | 1H |
| | CCAGTCTC | | | | | | |
| | ACCTCGGC | | | | | | |
| | TTGCAATG | | | | | | |
| | GACCCCAAC | | | | | | |
| 113 | CCCCTAGC | N | 077N | Hs00172870_m1 | NR2F6 | EAR- | nuclear |
| | ATGAACTTG | | | | | 2, EAR2, ERBAL2 | receptor |
| | TGGGATGG | | | | | | subfamily 2, |
| | TGGGGTTG | | | | | | group F, |
| | GCTTCCCT | | | | | | member 6 |
| | GGCATGATG | | | | | | |
| 114 | AGCCCAGG | P | 078P | Hs01122981_m1 | PJA2 | KIAA0438, | praja ring |
| | TCTAAATGT | | | | | Neurodap1, | finger 2 |
| | AATGGTTG | | | | | RNF131 | |
| | GTTTATTGT | | | | | | |
| | TCTATAACC | | | | | | |
| | CCAGCCC | | | | | | |
| 115 | CTGGATCA | N | 079N | Hs01937849_s1 | GPBAR1 | BG37, GPCR19, | G protein- |
| | GAGACCCT | | | | | GPR131, | coupled bile |
| | GCCTCTGTT | | | | | M- | acid receptor 1 |
| | TGACCCCG | | | | | BAR, MGC40597, | |
| | CACTGACT | | | | | TGR5 | |
| | GAATAAAGC | | | | | | |
| 116 | GCACGTGT | P | 079P | Hs00367579_m1 | CRLF3 | CREME9, | cytokine |
| | GTATCCAAT | | | | | CYTOR4, | receptor-like |
| | CTGCCTGT | | | | | FRWS, MGC20661, | factor 3 |
| | GACATGCA | | | | | p48.2 | |
| | TTTTACTCT | | | | | | |
| | TTGCAGAG | | | | | | |
| 117 | CGGTGGGT | N | 082N | Hs02379634_s1 | CH25H | C25H | cholesterol |
| | GCCCCTAA | | | | | | 25- |
| | GACTCGGG | | | | | | hydroxylase |
| | ACTGCTGT | | | | | | |
| | GCCTTTCAC | | | | | | |
| | ACTTGAATG | | | | | | |
| 118 | GTGCTGGG | P | 083P | Hs00233287_m1 | IKBKB | FLJ33771, | inhibitor of |
| | CCGGGGAG | | | | | FLJ36218, | kappa light |
| | TCCCTGTCT | | | | | FLJ38368, | polypeptide |
| | CTCACAGC | | | | | FLJ40509, | gene |
| | ATCTAGCA | | | | | IKK- | enhancer in |
| | GTATTATTA | | | | | beta, IKK2, | B-cells, kinase |
| | | | | | | IKKB, MGC131801, | beta |
| | | | | | | NFKBIKB | |
| 119 | CAGAGCCC | N | 084N | Hs00541730_m1 | CHST13 | C4ST3, MGC119278, | carbohydrate |
| | CTGGTGCA | | | | | MGC119279, | (chondroitin 4) |
| | ATGCGGTC | | | | | MGC119281 | sulfotransferase |
| | ACAGGTTTT | | | | | | 13 |
| | ATGGGACT | | | | | | |
| | TTGGTGAGC | | | | | | |
| 120 | CCCAAGTT | P | 084P | Hs00371639_m1 | SNUPN | KPNBL, RNUT1, | snurportin 1 |
| | GAAGGGTT | | | | | Snurportin1 | |
| | CTTCCCATA | | | | | | |
| | GCCCAGAC | | | | | | |
| | CACCCTGG | | | | | | |
| | ATGCCTCAT | | | | | | |
| 121 | CCTGGACT | N | 085N | Hs00384007_m1 | WSCD1 | KIAA0523 | WSC domain |
| | AAGCCAAT | | | | | | containing 1 |
| | GACACCTT | | | | | | |
| | CCATCTTTC | | | | | | |
| | CAGCTATG | | | | | | |
| | GTGACTGGG | | | | | | |
| 122 | ATGGGCAT | HSK | 01HSK | Hs02338565_gH | RPL19 | DKFZp779D216, | ribosomal |
| | AGGTAAGC | | | | | FLJ27452, MGC71997 | protein L19 |
| | GGAAGGGT | | | | | | |
| | ACAGCCAA | | | | | | |
| | TGCCCGAA | | | | | | |
| | TGCCAGAGAA | | | | | | |
| 123 | CCCCCAGT | HSK | 02HSK | Hs01086912_m1 | HNRNPD | AUF1, AUF1A, | heterogeneous |
| | ATTGTAGAG | | | | | HNRPD, | nuclear |
| | CAAGTCTTG | | | | | P37, hnRNPD0 | ribonucleoprotein |
| | TGTTAAAAG | | | | | | D (AU-rich |
| | CCCAGTGT | | | | | | element RNA |
| | GACAGTG | | | | | | binding |
| | | | | | | | protein 1, |
| | | | | | | | 37 kDa) |
| 124 | CGCGCGCA | HSK | 03HSK | Hs00828752_gH | RPS20 | FLJ27451, | ribosomal |
| | ACAGCCAT | | | | | MGC102930 | protein S20 |
| | GGCTTTTAA | | | | | | |
| | GGATACCG | | | | | | |
| | GAAAAACA | | | | | | |
| | CCCGTGGAG | | | | | | |
| 125 | TCATCTACA | HSK | 04HSK | Hs01631495_s1 | RPL26L1 | FLJ46904, | ribosomal |
| | TCGAGCGG | | | | | RPL26P1 | protein L26- |
| | GTGCAGCG | | | | | | like 1 |
| | TGAGAAGG | | | | | | |
| | CCAACGGC | | | | | | |
| | ACAACTGTC | | | | | | |
| 126 | GTGTTTAAC | HSK | 06HSK | Hs00371372_m1 | SON | BASS1, C21ORF50, | SON DNA |
| | CTAATGCTC | | | | | DBP- | binding |
| | AGCCTTGG | | | | | 5, FLJ21099, | protein |
| | TACTCCATT | | | | | FLJ33914, | |
| | CCCTTCTCC | | | | | HSPC310, | |
| | TTCCCC | | | | | KIAA1019, | |
| | | | | | | NREBP, | |
| | | | | | | SON3 | |
| 127 | GTGACTTCT | HSK | 07HSK | Hs00763191_s1 | YPEL5 | CGI-127 | yippee-like 5 |
| | GAGTACAG | | | | | | (Drosophila) |
| | TTAAGTTCC | | | | | | |
| | TCCTATTTG | | | | | | |
| | CCACTGGG | | | | | | |
| | CTGTTGG | | | | | | |
| 128 | TCACTCAAG | HSK | 08HSK | Hs01115161_m1 | DPP7 | DPP2, DPPII, | dipeptidyl- |
| | CAGCTGGC | | | | | QPP | peptidase 7 |
| | GGCAGAGG | | | | | | |
| | GAAGGGGC | | | | | | |
| | TGAATAAAC | | | | | | |
| | GCCTGGAG | | | | | | |
| 129 | ACTGAAAG | HSK | 10HSK | Hs00559413_m1 | ANXA7 | ANX7, RP11- | annexin A7 |
| | CTCTGCCTT | | | | | 537A6.8, | |
| | CCGGAATC | | | | | SNX, SYN | |
| | CCTCTAAGT | | | | | EXIN | |
| | CTGCTTGAT | | | | | | |
| | AGAGTGG | | | | | | |
| 130 | CTGAGGCT | HSK | 11HSK | Hs00212868_m1 | PAIP2 | HSPC218, | poly(A) |
| | ACAAGTTAG | | | | | MGC72018, | binding |
| | TCAGCAGA | | | | | PAIP2A | protein |
| | TGAGTGCC | | | | | | interacting |
| | AGTCCAGC | | | | | | protein 2 |
| | CTTTTCTGG | | | | | | |
| 131 | GTGGGGAC | HSK | 12HSK | Hs00199284_m1 | RAB35 | H- | RAB35, |
| | TCAGGGCT | | | | | ray, RAB1C, | member RAS |
| | GGACCGAC | | | | | RAY | oncogene |
| | GTCCTAGT | | | | | | family |
| | GGACCTGA | | | | | | |
| | TGTGAAATTC | | | | | | |
| 132 | CCGGCTCT | HSK | 13HSK | Hs00606477_m1 | CDC37 | P50CDC37 | cell division |
| | CGTCACTG | | | | | | cycle 37 |
| | GGCTCTGT | | | | | | homolog (S. cerevisiae) |
| | TTTCACTGT | | | | | | |
| | TCGTCTGCT | | | | | | |
| | GTCTGTGT | | | | | | |
| 133 | TGGCCTTTC | HSK | 14HSK | Hs00194538_m1 | SRSF4 | SFRS4, SRP75 | serine/arginine- |
| | CTACAGGG | | | | | | rich splicing |
| | AGCTCAGT | | | | | | factor 4 |
| | AACCTGGA | | | | | | |
| | CGGCTCTA | | | | | | |
| | AGGCTGGAA | | | | | | |
| 134 | CAAGCTTTC | HSK | 15HSK | Hs00363236_m1 | AKIRIN2 | C6ORF166, | akirin 2 |
| | GTCAGTGG | | | | | FBI1, FLJ10342, | |
| | CAACCACT | | | | | dJ486L4.2 | |
| | CTTAGGCA | | | | | | |
| | GCAGCAAC | | | | | | |
| | TGGTTTTGG | | | | | | |
| 135 | GGTCCTGG | HSK | 16HSK | Hs00229388_m1 | CCDC130 | MGC10471, | coiled-coil |
| | TGAGGGTG | | | | | SB115 | domain |
| | TTTGTGCCT | | | | | | containing |
| | TGTGAGAC | | | | | | 130 |
| | TCCGTACAT | | | | | | |
| | TAAAGACC | | | | | | |
| 136 | GCCCAGTT | HSK | 17HSK | Hs00272036_m1 | CD2BP2 | FWP010, | CD2 |
| | TGGTGGGC | | | | | LIN1, Snu40, | (cytoplasmic |
| | CCTTCTTTC | | | | | U5- | tail) binding |
| | CTGGACTTT | | | | | 52K | protein 2 |
| | GTGGAGGA | | | | | | |
| | GGCACCAA | | | | | | |
| 137 | TCCTCCCC | HSK | 18HSK | Hs00197056_m1 | TIMM23 | FLJ40725, | translocase of |
| | CATGAACTA | | | | | FLJ56773, | inner |
| | GAAAACCA | | | | | FLJ57459, | mitochondrial |
| | CTTACTCCC | | | | | FLJ79448, | membrane 23 |
| | AGAATTCAG | | | | | MGC71995, | homolog |
| | GTCGTGC | | | | | MGC87383, | (yeast), translocase |
| | | | | | | RP11- | of inner |
| | | | | | | 481A12.7, | mitochondrial |
| | | | | | | TIM23, RP11- | membrane 23 |
| | | | | | | 592B15.7, | homolog B |
| | | | | | | bA592B15.7 | (yeast) |
| 138 | CTTCTGTAA | HSK | 20HSK | Hs00193824_m1 | MED6 | NY-REN- | mediator |
| | CCTTTCCTC | | | | | 28 | complex |
| | TCCCGGAC | | | | | | subunit 6 |
| | TTGAGCAA | | | | | | |
| | CCTACACA | | | | | | |
| | CTCACATG | | | | | | |
| 139 | CATCAGGA | HSK | 21HSK | Hs00702452_s1 | NUDC | HNUDC, | nuclear |
| | GAAAGGCT | | | | | MNUDC, | distribution |
| | GGGTCTTG | | | | | NPD011 | gene C |
| | GGACCTTG | | | | | | homolog (A. nidulans) |
| | TCCTCCCC | | | | | | |
| | AGTTGGCC | | | | | | |
| | TA | | | | | | |
| 140 | ACCAGTTTT | HSK | 22HSK | Hs00220038_m1 | TMEM167B | AD- | transmembrane |
| | TACAGCCT | | | | | 020, C1ORF119, | protein |
| | CCTGGGTG | | | | | FLJ90710 | 167B |
| | GGTCGTCT | | | | | | |
| | TGACCCAA | | | | | | |
| | ACTCTTGTG | | | | | | |
| 141 | CTGGGGAG | HSK | 23HSK | Hs00229455_m1 | URM1 | C9ORF74, | ubiquitin |
| | ATACTTGAT | | | | | MGC2668, | related |
| | GGCGCGAA | | | | | RP11- | modifier 1 |
| | TGTCCGTTT | | | | | 339B21.4 | |
| | TCTCTCCCT | | | | | | |
| | TCCCACC | | | | | | |
| 142 | GGCTGTAA | HSK | 24HSK | Hs00412682_m1 | UBXN4 | FLJ23318, | UBX domain |
| | AATGAGAAT | | | | | KIAA0242, | protein 4 |
| | TCTGCCCC | | | | | KIAA2042, | |
| | CTCACCTCT | | | | | UBXD2, | |
| | TACCCCAG | | | | | UBXDC1, | |
| | TACTATTC | | | | | erasin | |
| 143 | CTCCAGCC | HSK | 25HSK | Hs00380814_m1 | FAM177A1 | C14ORF24, | family with |
| | TGGGCGAC | | | | | DKFZp686J1254, | sequence |
| | AGAGTGAG | | | | | FLJ38854 | similarity 177, |
| | ACTCCATCT | | | | | | member A1 |
| | TGGGGGGA | | | | | | |
| | AAAAAGTAT | | | | | | |

TABLE 2B
192 Exemplary Positive and Negative GVHD Predictor Genes and Housekeeping (“HSK”) Genes (RNA192)

| RNA192 | | | | | | | | | | |
|---|---|---|---|---|---|---|---|---|---|---|
| Index | | | | | | | | | | |
| (SEQ | | RNA1546 | | | Accession | | | | | P or N |
| ID NO: | | Index | | | No. Basic | | | | | Predictor |
| 1547- | RNA1538 | (SEQ | | Accession | (without | | | | Probe | or HSK |
| 1738) | Index | ID NO) | ProbeID | No. | decimal) | Gene Name | Symbol | Synonyms | Sequence | gene |
| 1547 | 1 | 1196 | 6280672 | NM_030938.2 | NM_030938 | transmembrane protein | TMEM49 | VMP1; | ATATTCCATC | N |
| | | | | | | 49 (TMEM49), mRNA. | | DKFZP566I133 | CTGCCCAAC | |
| | | | | | | | | | CCTTCCTCTC | |
| | | | | | | | | | CCATCCTCAA | |
| | | | | | | | | | AAAAGGGCC | |
| | | | | | | | | | AT | |
| 1548 | 2 | 12 | 4200575 | NM_014232.1 | NM_014232 | vesicle-associated | VAMP2 | SYB2; | GCCCAGAGA | P |
| | | | | | | membrane protein 2 | | VAMP-2; | GAGCTGTCCT | |
| | | | | | | (synaptobrevin 2) | | FLJ11460 | CTCATTGGGT | |
| | | | | | | (VAMP2), mRNA. | | | GAACTGATTG | |
| | | | | | | | | | AGGAAGGGT | |
| | | | | | | | | | CT | |
| 1549 | — | — | 7570326 | NM_000024.4 | NM_000024 | adrenergic, beta-2-, | ADRB2 | B2AR; | CAGCTGTGAA | N |
| | | | | | | receptor, surface | | BETA2AR; | CATGGACTCT | |
| | | | | | | (ADRB2), mRNA. | | BAR; | TCCCCCACTC | |
| | | | | | | | | ADRBR; | CTCTTATTTG | |
| | | | | | | | | ADRB2R | CTCACACGGG | |
| 1550 | — | — | 5270431 | NM_004538.3 | NM_004538 | nucleosome assembly | NAP1L3 | MB20; | CCAGCCCATA | P |
| | | | | | | protein 1-like 3 | | NPL3; | AGACTAAGG | |
| | | | | | | (NAP1L3), mRNA. | | MGC26312 | GTTTAAATCT | |
| | | | | | | | | | GCTTGCACTA | |
| | | | | | | | | | GCTGTGCCTTC | |
| 1551 | — | — | 4210754 | NM_001018069.1 | NM_001018069 | SERPINE1 mRNA | SERBP1 | CGI-55; | GTTCCTTTTG | N |
| | | | | | | binding protein 1 | | FLJ90489; | CTGCCCATTT | |
| | | | | | | (SERBP1), transcript | | DKFZp564M2423; | GGGAGTATG | |
| | | | | | | variant3, mRNA. | | CHD3IP; | TGGCAATTCC | |
| | | | | | | | | PAIRBP1; | TAGTGCTCTTG | |
| | | | | | | | | PAI-RBP1; | | |
| | | | | | | | | HABP4L | | |
| 1552 | — | — | 2810255 | NM_015989.3 | NM_015989 | cysteinesulfinic acid | CSAD | PCAP; | GCCTTTTTAG | P |
| | | | | | | decarboxylase | | MGC119355; | GCCACAGTG | |
| | | | | | | (CSAD), mRNA. | | MGC119354; | ACCTGCGCA | |
| | | | | | | | | MGC119357; | ATGTTTATAT | |
| | | | | | | | | CSD | GCTTTGACCT | |
| | | | | | | | | | AC | |
| 1553 | 3 | 461 | 830553 | NM_017455.2 | NM_017455 | neuroplastin (NPTN), | NPTN | SDFR1; | ACCTAACGGT | N |
| | | | | | | transcript variant alpha, | | GP55; | TCTCATGCGG | |
| | | | | | | mRNA. | | DKFZp686L2477; | TGCGTAATTG | |
| | | | | | | | | np65; np55; | TAGATGCATG | |
| | | | | | | | | GP65; | TACTTGTGTG | |
| | | | | | | | | SDR1; | | |
| | | | | | | | | MGC102805 | | |
| 1554 | 4 | 1256 | 6580711 | NM_001129.3 | NM_001129 | AE binding protein 1 | AEBP1 | FLJ33612; | TCAGCACATG | P |
| | | | | | | (AEBP1), mRNA. | | ACLP | GAAGGCCCC | |
| | | | | | | | | | TGGTATGGAC | |
| | | | | | | | | | ACTGAAAGGA | |
| | | | | | | | | | AGGGCTGGT | |
| | | | | | | | | | CC | |
| 1555 | — | — | 6840471 | NM_021601.3 | NM_021601 | CD79a molecule, | CD79A | MB-1; IGA | TCCTTAGTCA | N |
| | | | | | | immunoglobulin- | | | TATTCCCCCA | |
| | | | | | | associated alpha | | | GTGGGGGGT | |
| | | | | | | (CD79A), transcript | | | GGGAGGGTA | |
| | | | | | | variant 2, mRNA. | | | ACCTCACTCT | |
| | | | | | | | | | TC | |
| 1556 | — | — | 4280743 | NM_000997.3 | NM_000997 | ribosomal protein L37 | RPL37 | MGC99572 | CCTGCGTCA | P |
| | | | | | | (RPL37), mRNA. | | | CAGGGAAGC | |
| | | | | | | | | | AACCTACAGA | |
| | | | | | | | | | GAAGCAGCA | |
| | | | | | | | | | GCTCCCCAA | |
| | | | | | | | | | GAGA | |
| 1557 | 5 | 1309 | 6960594 | NM_145869.1 | NM_145869 | annexin A11 | ANXA11 | ANX11; | TCACAGTTCT | N |
| | | | | | | (ANXA11), transcript | | CAP50 | GGAGGCTGA | |
| | | | | | | variant c, mRNA. | | | GAAGATCGT | |
| | | | | | | | | | GAGGCTGCA | |
| | | | | | | | | | TCTGGCAAG | |
| | | | | | | | | | GGCC | |
| 1558 | — | — | 4590139 | NM_006297.1 | NM_006297 | X-ray repair | XRCC1 | RCC | ATCCCAGCTT | P |
| | | | | | | complementing | | | TGAGGAGGC | |
| | | | | | | defective repair in | | | CCTGATGGA | |
| | | | | | | Chinese hamster cells | | | CAACCCCTCC | |
| | | | | | | 1 (XRCC1), mRNA. | | | CTGGCATTCG | |
| | | | | | | | | | TT | |
| 1559 | — | — | 5340110 | NM_024921.2 | NM_024921 | premature ovarian | POF1B | FLJ22792; | TAGTGGCTG | N |
| | | | | | | failure, 1B (POF1B), | | POF | GGAAAAGGG | |
| | | | | | | mRNA. | | | GTGTGCGAG | |
| | | | | | | | | | GGGAACTGG | |
| | | | | | | | | | GGATGCTTAA | |
| | | | | | | | | | TGTG | |
| 1560 | — | — | 2940048 | NM_001003789.1 | NM_001003789 | RAB, member of RAS | RABL2B | | CACCTCGGG | P |
| | | | | | | oncogene family-like | | | GACAATTCCT | |
| | | | | | | 2B (RABL2B), | | | TGGGCTTCTC | |
| | | | | | | transcript variant 1, | | | CTGAGGTAAT | |
| | | | | | | mRNA. | | | GATTTACCCCC | |
| 1561 | 6 | 224 | 6480095 | NM_030918.5 | NM_030918 | sortingnexin family | SNX27 | MGC126873; | GACCCCCTTT | N |
| | | | | | | member27 (SNX27), | | MGC20471; | TAAGCCAGTG | |
| | | | | | | mRNA. | | MGC126871; | AGCTGGGCT | |
| | | | | | | | | MY014; | TCAGTTTTTC | |
| | | | | | | | | KIAA0488 | CCAGGCCAT | |
| | | | | | | | | | GC | |
| 1562 | 7 | 220 | 6400148 | NM_080430.2 | NM_080430 | selenoprotein M | SELM | MGC40146; | GAATACTTCT | P |
| | | | | | | (SELM), mRNA. | | SEPM | CTTGCTGAGA | |
| | | | | | | | | | GCCGATGCC | |
| | | | | | | | | | CGTCCCCGG | |
| | | | | | | | | | GCCAGCAGG | |
| | | | | | | | | | GAT | |
| 1563 | — | — | 1300671 | NM_005437.2 | NM_005437 | nuclear receptor | NCOA4 | RFG; | AGGAGCCTTT | N |
| | | | | | | coactivator 4 (NCOA4), | | ARA70; | CCAGTTATCT | |
| | | | | | | mRNA. | | DKFZp762E1112; | TGAGTTGCAG | |
| | | | | | | | | PTC3; | CTCTGTAGTT | |
| | | | | | | | | ELE1 | TCTTGAGGCC | |
| 1564 | 8 | 254 | 7610537 | NM_002129.2 | NM_002129 | high-mobility group box | HMGB2 | HMG2 | GCAAAAGTGA | P |
| | | | | | | 2 (HMGB2), mRNA. | | | AGCAGGAAA | |
| | | | | | | | | | GAAGGGCCC | |
| | | | | | | | | | TGGCAGGCC | |
| | | | | | | | | | AACAGGCTCA | |
| | | | | | | | | | AAG | |
| 1565 | 9 | 1535 | 6960278 | NM_178552.2 | NM_178552 | chromosome 22 open | C22ORF33 | EAN57; | CTCGGCTACA | N |
| | | | | | | reading frame 33 | | MGC35206; | ACATGCGGT | |
| | | | | | | (C22orf33), mRNA. | | cE81G9.2 | CAAACTTGTT | |
| | | | | | | | | | TCGAGGGGC | |
| | | | | | | | | | TGCTGAGGA | |
| | | | | | | | | | GAC | |
| 1566 | 10 | 1067 | 5560133 | NM_152468.3 | NM_152468 | transmembrane | TMC8 | EVIN2; | AAGCAGCTG | P |
| | | | | | | channel-like 8 (TMC8), | | EVER2; | GTGTGGCAG | |
| | | | | | | mRNA. | | EV2; | GTTCAGGAG | |
| | | | | | | | | MGC40121; | AAGTGGCAC | |
| | | | | | | | | MGC102701 | CTGGTGGAG | |
| | | | | | | | | | GACCT | |
| 1567 | — | — | 7560037 | NM_133471.1 | NM_133471 | KIAA1949 (KIAA1949), | KIAA1949 | HKMT1098 | GCCACGCTTA | N |
| | | | | | | mRNA. | | | CTTGCTGTGT | |
| | | | | | | | | | CTGCGTGGA | |
| | | | | | | | | | ATTCTCTCCT | |
| | | | | | | | | | CTGTCCCCTCC | |
| 1568 | — | — | 2030274 | NM_138923.1 | NM_138923 | TAF1 RNA polymerase | TAF1 | KAT4; | GACCCAAACA | P |
| | | | | | | II, TATA box binding | | CCG1; | ACCCCGCAT | |
| | | | | | | protein (TBP)- | | P250; | GCTTCAGGA | |
| | | | | | | associated factor, | | BA2R; | GAACACAAG | |
| | | | | | | 250 kDa (TAF1), | | TAFII250; | GATGGACAT | |
| | | | | | | transcript variant 2, | | NSCL2; | GGAA | |
| | | | | | | mRNA. | | TAF2A; OF; | | |
| | | | | | | | | DYT3; | | |
| | | | | | | | | CCGS | | |
| 1569 | 11 | 508 | 1230017 | NM_018367.4 | NM_018367 | phytoceramidase, | PHCA | FLJ11238; | GGATTCTAGG | N |
| | | | | | | alkaline (PHCA), | | APHC | TGGACATTAC | |
| | | | | | | mRNA. | | | AGAGTTGAAT | |
| | | | | | | | | | TCCTCACTAC | |
| | | | | | | | | | CCCCTCCCGC | |
| 1570 | — | — | 6940088 | NM_201554.1 | NM_201554 | diacylglycerol kinase, | DGKA | MGC42356; | GTACCAGAC | P |
| | | | | | | alpha 80 kDa (DGKA), | | DGK- | CTAAGTGACA | |
| | | | | | | transcript variant 4, | | alpha; | AGAGACTGG | |
| | | | | | | mRNA. | | DAGK1; | AAGTGGTTG | |
| | | | | | | | | MGC12821; | GGCTGGAGG | |
| | | | | | | | | DAGK | GTGC | |
| 1571 | — | — | 580240 | NM_145755.1 | NM_145755 | tetratricopeptide repeat | TTC21A | STI2; | AGGATGCAG | N |
| | | | | | | domain 21A (TTC21A), | | MGC70523; | TCACCAACTA | |
| | | | | | | mRNA. | | DKFZp686P18239; | CAAACTGGC | |
| | | | | | | | | MGC156293 | CTGGAAGTAC | |
| | | | | | | | | | AGTCATCACG | |
| | | | | | | | | | CC | |
| 1572 | 12 | 754 | 2940075 | NM_018571.5 | NM_018571 | amyotrophic lateral | ALS2CR2 | ILPIPA; | GCTGTCCCTT | P |
| | | | | | | sclerosis 2 (juvenile) | | ILPIP; | GGGAATGGG | |
| | | | | | | chromosome region, | | PAPK; | CCCTCAGAG | |
| | | | | | | candidate 2 | | MGC102916; | GACAGTGCTT | |
| | | | | | | (ALS2CR2), mRNA. | | CALS- | CCAAGTACAT | |
| | | | | | | | | 21; | CT | |
| | | | | | | | | PRO1038 | | |
| 1573 | — | — | 7400408 | NM_178471.1 | NM_178471 | G protein-coupled | GPR119 | hGPCR2; | GCTCTATCCT | N |
| | | | | | | receptor 119 | | GPCR2; | GGACCCCCT | |
| | | | | | | (GPR119), mRNA. | | MGC119957 | TCCTTATCAC | |
| | | | | | | | | | TGGCATTGTG | |
| | | | | | | | | | CAGGTGGCC | |
| | | | | | | | | | TG | |
| 1574 | 13 | 1197 | 6290021 | NM_002811.3 | NM_002811 | proteasome (prosome, | PSMD7 | P40; S12; | GCTCTCTGCC | P |
| | | | | | | macropain) 26S | | MOV34 | TCCGGTCACT | |
| | | | | | | subunit, non-ATPase, | | | CTTGCTGTGG | |
| | | | | | | 7 (PSMD7), mRNA. | | | TGCTACGTG | |
| | | | | | | | | | GAAGTGAATGG | |
| 1575 | — | — | 1010224 | NM_020654.3 | NM_020654 | SUMO1/sentrin | SENP7 | KIAA1707; | CTGTACTTCC | N |
| | | | | | | specific peptidase 7 | | MGC157730 | ACGTGACTG | |
| | | | | | | (SENP7), transcript | | | GGTGCTGAG | |
| | | | | | | variant 1, mRNA. | | | GGGAGTTAAA | |
| | | | | | | | | | GCCTCCCTG | |
| | | | | | | | | | GTG | |
| 1576 | — | — | 5700519 | NM_002082.2 | NM_002082 | G protein-coupled | GRK6 | FLJ32135; | TCCAAGAGCT | P |
| | | | | | | receptor kinase 6 | | GPRK6 | GAATGTCTTT | |
| | | | | | | (GRK6), transcript | | | GGGCTGGAT | |
| | | | | | | variant 2, mRNA. | | | GGCTCAGTTC | |
| | | | | | | | | | CCCCAGACC | |
| | | | | | | | | | TG | |
| 1577 | — | — | 7100615 | NM_001042472.1 | NM_001042472 | abhydrolase domain | ABHD12 | DKFZP434P106; | ACCTTGGCTA | N |
| | | | | | | containing 12 | | dJ965G21.2; | CAGGCACAA | |
| | | | | | | (ABHD12), transcript | | C20orf22; | ATACATTTAC | |
| | | | | | | variant 1, mRNA. | | ABHD12A; | AAGAGCCCT | |
| | | | | | | | | BEM46L2 | GAGCTGCCA | |
| | | | | | | | | | CGG | |
| 1578 | — | — | 2260615 | NM_004698.1 | NM_004698 | PRP3 pre-mRNA | PRPF3 | HPRP3P; | GCATGGGGC | P |
| | | | | | | processing factor 3 | | HPRP3; | TGAACACTAC | |
| | | | | | | homolog (S. cerevisiae) | | Prp3p; | TGGGACCTT | |
| | | | | | | (PRPF3), | | RP18; | GCGCTGAGT | |
| | | | | | | mRNA. | | PRP3 | GAATCTGTGT | |
| | | | | | | | | | TAG | |
| 1579 | — | — | 4200458 | NM_005249.3 | NM_005249 | forkhead box G1 | FOXG1 | FKHL1; | GTTGTTTCAG | N |
| | | | | | | (FOXG1), mRNA. | | KHL2; | TTGGCAACAC | |
| | | | | | | | | HFK3; | TGCCCATTCA | |
| | | | | | | | | HBF2; | ATTGAATCAG | |
| | | | | | | | | FOXG1C; | AAGGGGACAA | |
| | | | | | | | | QIN; | | |
| | | | | | | | | FKHL2; | | |
| | | | | | | | | HBF-2; | | |
| | | | | | | | | HBF-1; | | |
| | | | | | | | | FKH2; | | |
| | | | | | | | | HFK1; | | |
| | | | | | | | | FKHL4; | | |
| | | | | | | | | HBF-G2; | | |
| | | | | | | | | BF2; | | |
| | | | | | | | | FHKL3; | | |
| | | | | | | | | BF1; HFK2; | | |
| | | | | | | | | HBF-3; | | |
| | | | | | | | | FOXG1B; | | |
| | | | | | | | | FKHL3; | | |
| | | | | | | | | FOXG1A | | |
| 1580 | — | — | 4810333 | NM_153701.1 | NM_153701 | interleukin 12 receptor, | IL12RB1 | CD212; IL- | GGGAAGATG | P |
| | | | | | | beta 1 (IL12RB1), | | 12R- | CCCTATCTCT | |
| | | | | | | transcript variant 2, | | BETA1; | CGGGTGCTG | |
| | | | | | | mRNA. | | IL12RB; | CCTACAACGT | |
| | | | | | | | | MGC34454 | GGCTGTCATC | |
| | | | | | | | | | TC | |
| 1581 | 14 | 406 | 540446 | NM_012459.1 | NM_012459 | translocase of inner | TIMM8B | MGC102866; | GGACTTGTTA | N |
| | | | | | | mitochondrial | | TIM8B; | CTAAGCAGAT | |
| | | | | | | membrane 8 homolog | | DDP2; | TTAAGGGTCA | |
| | | | | | | B (yeast) (TIMM8B), | | MGC117373; | GTGGGGGAA | |
| | | | | | | nuclear gene encoding | | FLJ21744 | GGCTATCAACC | |
| | | | | | | mitochondrial protein, | | | | |
| | | | | | | mRNA. | | | | |
| 1582 | — | — | 1820035 | NM_001077268.1 | NM_001077268 | zinc finger, FYVE | ZFYVE19 | FLJ14840; | GATAGGCCC | P |
| | | | | | | domain containing 19 | | MPFYVE | CTTCCTGAGC | |
| | | | | | | (ZFYVE19), mRNA. | | | CTTGGTGTCC | |
| | | | | | | | | | CTGGAATGA | |
| | | | | | | | | | GGAAAGATTC | |
| | | | | | | | | | TC | |
| 1583 | — | — | 6770168 | NM_006371.3 | NM_006371 | cartilage associated | CRTAP | CASP; OI7 | TCCCACTTTA | N |
| | | | | | | protein (CRTAP), | | | GGGTGGCAG | |
| | | | | | | mRNA. | | | CCAGTAGGC | |
| | | | | | | | | | CAAACTCCAA | |
| | | | | | | | | | AGACCGTTG | |
| | | | | | | | | | CTG | |
| 1584 | 15 | 658 | 2260296 | NR_003654.1 | NR_003654 | SCAN domain | SCAND2 | | GAACCAGTA | P |
| | | | | | | containing 2 | | | GTCCAGGGT | |
| | | | | | | (SCAND2) on | | | GGCTCACAAA | |
| | | | | | | chromosome 15. | | | GACCACTTTG | |
| | | | | | | | | | AGGCTCTTGC | |
| | | | | | | | | | TC | |
| 1585 | — | — | 5360376 | NM_006762.1 | NM_006762 | lysosomal associated | LAPTM5 | MGC125860; | CCACAGGTTA | N |
| | | | | | | multispanning | | MGC125861 | GTTCAGTCAA | |
| | | | | | | membrane protein 5 | | | AGCAGGCAA | |
| | | | | | | (LAPTM5), mRNA. | | | CCCCCTTGTG | |
| | | | | | | | | | GGCACTGAC | |
| | | | | | | | | | CC | |
| 1586 | 16 | 172 | 4830113 | NM_016619.1 | NM_016619 | placenta-specific 8 | PLAC8 | C15; onzin | TAAGGCCCT | P |
| | | | | | | (PLAC8), mRNA. | | | GCACTGAAAA | |
| | | | | | | | | | TGCAAGCTCA | |
| | | | | | | | | | GGCGCCGGT | |
| | | | | | | | | | GGTCGTTGT | |
| | | | | | | | | | GAC | |
| 1587 | — | — | 4850082 | NM_003780.3 | NM_003780 | UDP-Gal:betaGlcNAc | B4GALT2 | beta4Gal- | GATCTTGGG | N |
| | | | | | | beta 1,4- | | T2; B4Gal- | GTTGGCCTTT | |
| | | | | | | galactosyltransferase, | | T3; B4Gal- | GCATGGGAG | |
| | | | | | | polypeptide 2 | | T2 | GCAGGTGGG | |
| | | | | | | (B4GALT2), transcript | | | GCTTGGATCA | |
| | | | | | | variant 2, mRNA. | | | GTA | |
| 1588 | — | — | 2970017 | NM_005978.3 | NM_005978 | S100 calcium binding | S100A2 | S100L; | CTCAGCTGG | P |
| | | | | | | protein A2 (S100A2), | | CAN19; | AGTGCTGGG | |
| | | | | | | mRNA. | | MGC111539 | AGATGAGGG | |
| | | | | | | | | | CCTCCTGGAT | |
| | | | | | | | | | CCTGCTCCCT | |
| | | | | | | | | | TCT | |
| 1589 | 17 | 1357 | 7380601 | NM_024896.2 | NM_024896 | endoplasmic reticulum | ERMP1 | FXNA; | GATAGGATTC | N |
| | | | | | | metallopeptidase 1 | | KIAA1815; | CTTAAGATGT | |
| | | | | | | (ERMP1), mRNA. | | bA207C16.3 | TACCACCCAG | |
| | | | | | | | | | GGGGCCACA | |
| | | | | | | | | | AGCCAGCCT | |
| | | | | | | | | | GC | |
| 1590 | — | — | 1110575 | NM_002494.2 | NM_002494 | NADH dehydrogenase | NDUFC1 | MGC138266; | GGCCCTTCA | P |
| | | | | | | (ubiquinone) 1, | | KFYI; | GTGCGATCAA | |
| | | | | | | subcomplex unknown, | | MGC126847; | AGTTCTACGT | |
| | | | | | | 1, 6 kDa (NDUFC1), | | MGC117464 | GCGAGAGCC | |
| | | | | | | mRNA. | | | GCCGAATGC | |
| | | | | | | | | | CAA | |
| 1591 | — | — | 1740382 | NM_000161.2 | NM_000161 | GTP cyclohydrolase 1 | GCH1 | DYT5; | TGCACAAAAC | N |
| | | | | | | (dopa-responsive | | GTP-CH-1; | CACTGCCAG | |
| | | | | | | dystonia) (GCH1), | | GTPCH1; | ATAACCAGAG | |
| | | | | | | transcript variant 1, | | GCH | GGGCCTGGG | |
| | | | | | | mRNA. | | | AAGGGAGAA | |
| | | | | | | | | | GAA | |
| 1592 | — | — | 780184 | NM_006346.2 | NM_006346 | progesteroneimmunomodulatory | PIBF1 | RP11- | GTCCACTACG | P |
| | | | | | | binding factor | | 505F3.1; | AGGTACTTCA | |
| | | | | | | 1 (PIBF1), mRNA. | | KIAA1008; | AAAGCCCAGT | |
| | | | | | | | | PIBF1; | AATGGTGGTC | |
| | | | | | | | | C13orf24 | AGATACCATG | |
| 1593 | 18 | 757 | 2970397 | NM_145288.1 | NM_145288 | zinc finger protein 342 | ZNF342 | ZNF296 | GTACCGCTG | N |
| | | | | | | (ZNF342), mRNA. | | | CCAACACCCA | |
| | | | | | | | | | TTGACCTCCT | |
| | | | | | | | | | CGTTTTTGCC | |
| | | | | | | | | | CGCCTTCTCCA | |
| 1594 | — | — | 3610280 | NM_016446.2 | NM_016446 | chromosome 9 open | C9ORF127 | NGX6; | TTCCACCACG | P |
| | | | | | | reading frame 127 | | RP11- | TTCTCCGAGG | |
| | | | | | | (C9orf127), mRNA. | | 112J3.10; | GTTTGGGAAT | |
| | | | | | | | | NAG-5; | GTCTGTGCCT | |
| | | | | | | | | MGC120460 | TCACTGTGTC | |
| 1595 | 19 | 237 | 6960593 | NM_004439.4 | NM_004439 | EPH receptor A5 | EPHA5 | EHK1; | CTGTGGGAG | N |
| | | | | | | (EPHA5), transcript | | TYRO4; | GGCTTCTTCC | |
| | | | | | | variant 1, mRNA. | | HEK7; | CTGTGCGCT | |
| | | | | | | | | CEK7 | GTTGCCCATC | |
| | | | | | | | | | CAAGCCTAAT | |
| | | | | | | | | | AT | |
| 1596 | 20 | 1330 | 7150685 | NM_012117.1 | NM_012117 | chromobox homolog 5 | CBX5 | HP1- | TCAGAGGAT | P |
| | | | | | | (HP1 alpha homolog, | | ALPHA; | GAGGAGGAG | |
| | | | | | | Drosophila) (CBX5), | | HP1; | TATGTTGTGG | |
| | | | | | | mRNA. | | HP1Hs- | AGAAGGTGC | |
| | | | | | | | | alpha | TAGACAGGC | |
| | | | | | | | | | GCGT | |
| 1597 | 21 | 570 | 1660072 | NM_130787.2 | NM_130787 | adaptor-related protein | AP2A1 | CLAPA1; | CTCCACTGGT | N |
| | | | | | | complex 2, alpha 1 | | AP2- | GACAGAGAA | |
| | | | | | | subunit (AP2A1), | | ALPHA; | GACACCAGG | |
| | | | | | | transcript variant 2, | | ADTAA | GTTTGGGGG | |
| | | | | | | mRNA. | | | ATGCCTGGG | |
| | | | | | | | | | ACTT | |
| 1598 | — | — | 1510035 | NM_001981.2 | NM_001981 | epidermal growth | EPS15 | AF1P; | CTGGCTCCA | P |
| | | | | | | factor receptor | | MLLT5; AF- | GGGCCTGTG | |
| | | | | | | pathway substrate 15 | | 1P | CTTGAAAAGG | |
| | | | | | | (EPS15), mRNA. | | | ACAGATAAGT | |
| | | | | | | | | | ATTGCCCAGA | |
| | | | | | | | | | GC | |
| 1599 | — | — | 6520605 | NM_005871.2 | NM_005871 | survival motor neuron | SMNDC1 | SPF30; | CAGGTTGTCT | N |
| | | | | | | domain containing 1 | | SMNR | GCATTTGTTG | |
| | | | | | | (SMNDC1), mRNA. | | | GTGTAAGTGA | |
| | | | | | | | | | ACATCATCAC | |
| | | | | | | | | | AGTTATCCTG | |
| 1600 | — | — | 6250288 | NM_022474.2 | NM_022474 | membrane protein, | MPP5 | FLJ12615; | CCCTCTGTG | P |
| | | | | | | palmitoylated 5 | | PALS1 | GTTCTGACTG | |
| | | | | | | (MAGUK p55 | | | GAGACCCCA | |
| | | | | | | subfamily member 5) | | | GTGTGGGGG | |
| | | | | | | (MPP5), mRNA. | | | AGGTCTTACC | |
| | | | | | | | | | ATT | |
| 1601 | 22 | 207 | 6060196 | NM_145912.5 | NM_145912 | NFAT activating | NFAM1 | FLJ40652; | GGGACTCAG | N |
| | | | | | | protein with ITAM motif | | CNAIP; | CATTTTCCAG | |
| | | | | | | 1 (NFAM1), mRNA. | | bK126B4.4 | TCTTTTTCAG | |
| | | | | | | | | | GGGTAGACA | |
| | | | | | | | | | GGGGAGCCT | |
| | | | | | | | | | GGG | |
| 1602 | — | — | 3420767 | NM_016173.3 | NM_016173 | HemKmethyltransferase | HEMK1 | FLJ22320; | AATTGCTGGA | P |
| | | | | | | family member 1 | | HEMK; | GGAAGAGCC | |
| | | | | | | (HEMK1), mRNA. | | MTQ1 | ATGAGCCGA | |
| | | | | | | | | | GGAATGCAG | |
| | | | | | | | | | ACAGCCTCTT | |
| | | | | | | | | | CTC | |
| 1603 | — | — | 7510386 | NM_173843.1 | NM_173843 | interleukin 1 receptor | IL1RN | ICIL-1RA; | GGCACTTGG | N |
| | | | | | | antagonist (IL1RN), | | IRAP; IL- | AGACTTGTAT | |
| | | | | | | transcript variant 4, | | 1ra3; | GAAAGATGG | |
| | | | | | | mRNA. | | MGC10430; | CTGTGCCTCT | |
| | | | | | | | | IL1F3; | GCCTGTCTCC | |
| | | | | | | | | IL1RA | CC | |
| 1604 | 23 | 14 | 5220196 | NM_006565.2 | NM_006565 | CCCTC-binding factor | CTCF | | ATGTAGCAGA | P |
| | | | | | | (zinc finger protein) | | | ATGGCACCC | |
| | | | | | | (CTCF), mRNA. | | | AGACCACTG | |
| | | | | | | | | | CCCACCAGT | |
| | | | | | | | | | GACGGACAT | |
| | | | | | | | | | GCAC | |
| 1605 | 24 | 1329 | 7150278 | NM_000201.1 | NM_000201 | intercellular adhesion | ICAM1 | P3.58; BB2; | GCAGTGATCA | N |
| | | | | | | molecule 1 (CD54), | | CD54 | GGGTCCTGC | |
| | | | | | | human rhinovirus | | | AAGCAGTGG | |
| | | | | | | receptor (ICAM1), | | | GGAAGGGGG | |
| | | | | | | mRNA. | | | CCAAGGTATT | |
| | | | | | | | | | GGA | |
| 1606 | 25 | 271 | 2510253 | NM_145306.2 | NM_145306 | chromosome 10 open | C1CORF35 | | ACATGTTCCG | P |
| | | | | | | reading frame 35 | | | ATGCCTGTG | |
| | | | | | | (C10orf35), mRNA. | | | GAAGACATG | |
| | | | | | | | | | CCGACGTCT | |
| | | | | | | | | | CCTCTGCCTA | |
| | | | | | | | | | GGG | |
| 1607 | — | — | 3610440 | NM_005360.3 | NM_005360 | v- | MAF | MGC71685 | GCAGCGACA | N |
| | | | | | | mafmusculoaponeuroticfibrosarcoma | | | ACCCGTCCTC | |
| | | | | | | oncogene homolog | | | TCCCGAGTTT | |
| | | | | | | (avian) (MAF), | | | TTCATAACTG | |
| | | | | | | transcript variant 1, | | | AGCCCACTC | |
| | | | | | | mRNA. | | | GC | |
| 1608 | 26 | 457 | 830324 | NM_001459.2 | NM_001459 | fms-related tyrosine | FLT3LG | | ACACAGAGG | P |
| | | | | | | kinase 3 ligand | | | AAGTTGGCTA | |
| | | | | | | (FLT3LG), mRNA. | | | GAGGCCGGT | |
| | | | | | | | | | CCCTTCCTTG | |
| | | | | | | | | | GGCCCCTCT | |
| | | | | | | | | | CAT | |
| 1609 | 27 | 1042 | 5290008 | NM_015112.2 | NM_015112 | microtubule associated | MAST2 | FLJ39200; | TCAGGAGGG | N |
| | | | | | | serine/threonine kinase | | RP4- | GCCAAGAAC | |
| | | | | | | 2 (MAST2), mRNA. | | 533D7.1; | CAGGGGGCC | |
| | | | | | | | | KIAA0807; | ATCAAAAGCA | |
| | | | | | | | | MAST205; | TCGGGATTTG | |
| | | | | | | | | MTSSK | GCA | |
| 1610 | 28 | 231 | 6650451 | NM_015057.3 | NM_015057 | MYC binding protein 2 | MYCBP2 | FLJ21597; | GAGGTGTTTG | P |
| | | | | | | (MYCBP2), mRNA. | | PAM; | CATGTGGCC | |
| | | | | | | | | FLJ13826; | ATTACCGTCA | |
| | | | | | | | | FLJ10106; | TTGGCCTGTG | |
| | | | | | | | | FLJ21646; | AAGCATTGGAC | |
| | | | | | | | | DKFZp686M08244; | | |
| | | | | | | | | KIAA0916 | | |
| 1611 | — | — | 4150538 | NM_144675.1 | NM_144675 | GSG1-like (GSG1L), | GSG1L | MGC18079; | AGGGCAGGC | N |
| | | | | | | mRNA. | | PRO19651 | CCAAGGGAA | |
| | | | | | | | | | TGCACAGGG | |
| | | | | | | | | | CTGCACAGA | |
| | | | | | | | | | GTGACTTTGG | |
| | | | | | | | | | GACA | |
| 1612 | — | — | 2450102 | NM_201438.1 | NM_201438 | periphilin 1 (PPHLN1), | PPHLN1 | HSPC206; | TGTTTGAGTT | P |
| | | | | | | transcript variant 5, | | HSPC232; | GACTTCACAG | |
| | | | | | | mRNA. | | MGC48786 | TCAGTTTGAT | |
| | | | | | | | | | CAGTATGGTC | |
| | | | | | | | | | CCCCACCTGG | |
| 1613 | 29 | 774 | 3140095 | NM_177543.1 | NM_177543 | phosphatidic acid | PPAP2C | PAP-2c; | AGGCTCGGG | N |
| | | | | | | phosphatase type 2C | | PAP2-g; | GGTCCCCGC | |
| | | | | | | (PPAP2C), transcript | | LPP2 | GTCCCAGGC | |
| | | | | | | variant 3, mRNA. | | | CCAGGGGGA | |
| | | | | | | | | | TGGGGGTCG | |
| | | | | | | | | | CGAGA | |
| 1614 | — | — | 450398 | NM_004798.2 | NM_004798 | kinesin family member | KIF3B | HH0048; | TGCTGCAACT | P |
| | | | | | | 3B (KIF3B), mRNA. | | KIAA0359 | GGGGCGTGG | |
| | | | | | | | | | GCCGCTCTCT | |
| | | | | | | | | | GCTTTTCCTG | |
| | | | | | | | | | TCTGACTCTGA | |
| 1615 | — | — | 4810458 | NM_181309.1 | NM_181309 | interleukin 22 receptor, | IL22RA2 | CRF2-S1; | GGGGGTGGA | N |
| | | | | | | alpha 2 (IL22RA2), | | MGC150509; | GGAGAATAA | |
| | | | | | | transcript variant 2, | | IL-22BP; | GAGGCAGAG | |
| | | | | | | mRNA. | | MGC150510; | CAAGAGCTA | |
| | | | | | | | | CRF2- | GAGAATTGGT | |
| | | | | | | | | 10; CRF2X | TTCC | |
| 1616 | — | — | 3890196 | NM_152850.2 | NM_152850 | phosphatidylinositol | PIGO | RP11- | CCTGGGCAT | P |
| | | | | | | glycan anchor | | 182N22.4; | AGCTTTGGTG | |
| | | | | | | biosynthesis, class O | | DKFZp434M222; | ATGAGAGTG | |
| | | | | | | (PIGO), transcript | | FLJ00135; | GATGGTGCT | |
| | | | | | | variant 2, mRNA. | | MGC3079; | GTGAGCTCCT | |
| | | | | | | | | MGC20536 | GGT | |
| 1617 | — | — | 1230156 | NM_004155.3 | NM_004155 | serpin peptidase | SERPINB9 | PI9; CAP-3; | CAGACAAACT | N |
| | | | | | | inhibitor, clade B | | CAP3 | GTTTTCCACA | |
| | | | | | | (ovalbumin), member 9 | | | GCAGTTGAAC | |
| | | | | | | (SERPINB9), mRNA. | | | CATTCCACAT | |
| | | | | | | | | | TCCCACCAGC | |
| 1618 | — | — | 1190138 | NM_003328.2 | NM_003328 | TXK tyrosine kinase | TXK | MGC22473; | GTAGCCAAA | P |
| | | | | | | (TXK), mRNA | | PSCTK5; | GCTCACCTTT | |
| | | | | | | | | PTK4; | GAACAGATCC | |
| | | | | | | | | BTKL; TKL; | CGGTGACATT | |
| | | | | | | | | RLK | CTATTTCCAGG | |
| 1619 | — | — | 3780139 | NM_020820.3 | NM_020820 | phosphatidylinositol | PREX1 | KIAA1415 | GGCAGTTTGT | N |
| | | | | | | 3,4,5-trisphosphate- | | | CCCCCCAGC | |
| | | | | | | dependent RAC | | | TTCGGTATGC | |
| | | | | | | exchanger 1 (PREX1), | | | CTTCAGGGAA | |
| | | | | | | mRNA. | | | AGGTCACAG | |
| | | | | | | | | | CT | |
| 1620 | 30 | 351 | 130241 | NM_001007468.1 | NM_001007468 | SWI/SNF related, | SMARCB1 | Sfh1p; | TACGCCTTCA | P |
| | | | | | | matrix associated, actin | | RDT; | GCGAGAACC | |
| | | | | | | dependent regulator of | | hSNFS; | CTCTGCCCAC | |
| | | | | | | chromatin, subfamily b, | | SNF5; | AGTGGAGATT | |
| | | | | | | member 1 | | Snr1; | GCCATCCGG | |
| | | | | | | (SMARCB1), transcript | | SNF5L1; | AA | |
| | | | | | | variant 2, mRNA. | | INI1; | | |
| | | | | | | | | BAF47 | | |
| 1621 | — | — | 5340458 | NM_018044.2 | NM_018044 | NOL1/NOP2/Sun | NSUN5 | p120; | ACGTGCTCC | N |
| | | | | | | domain family, | | FLJ10267; | CTCTGCCAG | |
| | | | | | | member 5 (NSUN5), | | WBSCR20; | GAGGAGAAT | |
| | | | | | | transcript variant 2, | | p120 | GAAGACGTG | |
| | | | | | | mRNA. | | (NOL1); | GTGCGAGAT | |
| | | | | | | | | MGC986; | GCGCT | |
| | | | | | | | | WBSCR20A; | | |
| | | | | | | | | (NOL1); | | |
| | | | | | | | | NOL1R; | | |
| | | | | | | | | NSUN5A | | |
| 1622 | — | — | 70608 | NM_172177.1 | NM_172177 | mitochondrial | MRPL42 | PTD007; | GACAGATGAT | P |
| | | | | | | ribosomal protein L42 | | MRPS32; | GCGGAGGTT | |
| | | | | | | (MRPL42), nuclear | | MRP-L31; | CCTGGGGGA | |
| | | | | | | gene encoding | | RPML31; | ATCAAAGAGA | |
| | | | | | | mitochondrial protein, | | HSPC204 | AATGTGCCTC | |
| | | | | | | transcript variant 2, | | | AT | |
| | | | | | | mRNA. | | | | |
| 1623 | — | — | 3460053 | NM_020808.3 | NM_020808 | signal-induced | SIPA1L2 | SPAL2; | TCAGACCGA | N |
| | | | | | | proliferation-associated | | FLJ23126; | GAAGCAGGG | |
| | | | | | | 1 like 2 (SIPA1L2), | | KIAA1389; | TGAGAGATTC | |
| | | | | | | mRNA. | | FLJ23632 | TAACGACTGG | |
| | | | | | | | | | ATGCTGCTAG | |
| | | | | | | | | | TA | |
| 1624 | — | — | 6220343 | NM_021098.2 | NM_021098 | calcium channel, | CACNA1H | CACNA1HB; | ATCAGGCCTC | P |
| | | | | | | voltage-dependent, T | | FLJ90484; | CCCTACATCT | |
| | | | | | | Type, alpha 1H subunit | | Cav3.2 | GGGGGCGTT | |
| | | | | | | (CACNA1H), transcript | | | GGCCGCGAG | |
| | | | | | | variant 1, mRNA. | | | ATTCCCATTG | |
| | | | | | | | | | AC | |
| 1625 | — | — | 6620753 | NM_006007.1 | NM_006007 | zinc finger, AN1-type | ZFAND5 | ZFAND5A; | TGTACTTGGG | N |
| | | | | | | domain 5 (ZFAND5), | | ZA20D2; | TGTAGGACTC | |
| | | | | | | mRNA. | | ZNF216 | TAGTGTTCTT | |
| | | | | | | | | | GGGTGTATTG | |
| | | | | | | | | | CATGGGCTGC | |
| 1626 | — | — | 5860605 | NM_013374.3 | NM_013374 | programmed cell death | PDCD6IP | MGC17003; | AGAGCCTTGT | P |
| | | | | | | 6 interacting protein | | Alix; | GTCCCTAAAG | |
| | | | | | | (PDCD6IP), mRNA. | | DRIP4; | TTCTGTCCCA | |
| | | | | | | | | AIP1; HP95 | GTCAGCAGT | |
| | | | | | | | | | CTTTATAGTCC | |
| 1627 | — | — | 160390 | NM_001014839.1 | NM_001014839 | neurochondrin | NCDN | KIAA0607 | AGGCCTGGT | N |
| | | | | | | (NCDN), transcript | | | GGGGGGTGG | |
| | | | | | | variant 1, mRNA. | | | GGAAACCTC | |
| | | | | | | | | | CTTCCACCTG | |
| | | | | | | | | | AGCTTGCTTG | |
| | | | | | | | | | AAG | |
| 1628 | — | — | 3890136 | NM_006370.1 | NM_006370 | vesicle transport | VTI1B | VTI1; | GAGCTGGGG | P |
| | | | | | | through interaction with | | VTI1L; VTI2 | GAACAACGA | |
| | | | | | | t-SNAREs homolog 1B | | | GACCAGTTAG | |
| | | | | | | (yeast) (VTI1B), | | | AACGTACCAA | |
| | | | | | | mRNA. | | | GAGTAGACT | |
| | | | | | | | | | GGT | |
| 1629 | 31 | 181 | 5090288 | NM_171999.2 | NM_171999 | sal-like 3 (Drosophila) | SALL3 | ZNF796 | GTGGTCTGTA | N |
| | | | | | | (SALL3), mRNA. | | | GCCCAATAAC | |
| | | | | | | | | | TGGGGAACG | |
| | | | | | | | | | AGTTACAGAC | |
| | | | | | | | | | AAACATCACCG | |
| 1630 | — | — | 2750592 | NM_032026.2 | NM_032026 | TatDDNase domain | TATDN1 | CDA11 | CGGTGTGTA | P |
| | | | | | | containing 1 | | | GGGGGAGTG | |
| | | | | | | (TATDN1), mRNA. | | | GTGCATTCAT | |
| | | | | | | | | | TTGATGGTAC | |
| | | | | | | | | | CAAGGAAGC | |
| | | | | | | | | | AGC | |
| 1631 | — | — | 7330435 | NM_005436.2 | NM_005436 | coiled-coil domain | CCDC6 | TST1; TPC; | AGAGGAGAG | N |
| | | | | | | containing 6 (CCDC6), | | PTC; H4; | CCAAGCGCT | |
| | | | | | | mRNA. | | D10S170; | AGCATGCCTT | |
| | | | | | | | | FLJ32286 | TTGCCTCTGC | |
| | | | | | | | | | ATATCTGTGC | |
| | | | | | | | | | AC | |
| 1632 | — | — | 7570500 | NM_032314.3 | NM_032314 | coenzyme Q5 | COQ5 | MGC4767; | GGAGCTGCT | P |
| | | | | | | homolog, | | MGC104303 | TCAGTCCATC | |
| | | | | | | methyltransferase (S. cerevisiae) | | | TCCCAGAGG | |
| | | | | | | (COQ5), | | | CATTTGGTCT | |
| | | | | | | mRNA. | | | GTATCTTTGC | |
| | | | | | | | | | TC | |
| 1633 | — | — | 6020612 | NM_002158.3 | NM_002158 | forkhead box N2 | FOXN2 | HTLF | ACTGCCAGTA | N |
| | | | | | | (FOXN2), mRNA. | | | GATGACCAGT | |
| | | | | | | | | | CACAAGTGAA | |
| | | | | | | | | | CCACTTCTCA | |
| | | | | | | | | | GTTGCCAATC | |
| 1634 | — | — | 840240 | NM_007124.2 | NM_007124 | utrophin (UTRN), | UTRN | DRP; | ACCGCACGA | P |
| | | | | | | mRNA. | | DMDL; | CACCAGCAC | |
| | | | | | | | | DRP1; | GGATCTCAC | |
| | | | | | | | | FLJ23678 | GGAGGTCAT | |
| | | | | | | | | | GGAGCAGAT | |
| | | | | | | | | | TCACA | |
| 1635 | — | — | 6450176 | NM_138711.3 | NM_138711 | peroxisome | PPARG | NR1C3; | GGTGGGTGT | N |
| | | | | | | proliferator-activated | | PPARG1; | GTAGTCGTG | |
| | | | | | | receptor gamma | | PPARG2 | GTACTTTACG | |
| | | | | | | (PPARG), transcript | | | CCTCGGTGTT | |
| | | | | | | variant 3, mRNA. | | | TAGGGAGGA | |
| | | | | | | | | | GCC | |
| 1636 | — | — | 4060669 | NM_019083.1 | NM_019083 | coiled-coil domain | CCDC76 | FLJ10287; | GTCTCGGTTC | P |
| | | | | | | containing 76 | | FLJ11219 | AAATTCCAAA | |
| | | | | | | (CCDC76), mRNA. | | | CCTACCATCT | |
| | | | | | | | | | TCAGTTGTGC | |
| | | | | | | | | | GACCTTGGGC | |
| 1637 | — | — | 10220 | NM_001002246.1 | NM_001002246 | APC11 anaphase | ANAPC11 | HSPC214; | AGGCCTCTG | N |
| | | | | | | promoting complex | | MGC882; | GGTGCCTGT | |
| | | | | | | subunit 11 homolog | | Apc11p; | GTTCTCGGCA | |
| | | | | | | (yeast) (ANAPC11), | | APC11 | TATAGATGTG | |
| | | | | | | transcript variant 4, | | | GTCTCGGTGT | |
| | | | | | | mRNA. | | | GT | |
| 1638 | — | — | 770541 | NM_001007277.1 | NM_001007277 | etoposide induced 2.4 | EI24 | TP53I8; | CACTAAACCT | P |
| | | | | | | mRNA (EI24), | | PIG8 | GAACTTTTCA | |
| | | | | | | transcript variant 2, | | | ACTCCGTTGG | |
| | | | | | | mRNA. | | | TGGTGGGAG | |
| | | | | | | | | | GCAGCGGGC | |
| | | | | | | | | | AG | |
| 1639 | — | — | 7160270 | NM_004450.1 | NM_004450 | enhancer of | ERH | FLJ27340; | GGTTGGGGT | N |
| | | | | | | rudimentary homolog | | DROER | GGGCTTGGA | |
| | | | | | | (Drosophila) (ERH), | | | ACACAGGTGT | |
| | | | | | | mRNA. | | | GTACAGCGT | |
| | | | | | | | | | GCTGTAGTG | |
| | | | | | | | | | GAAG | |
| 1640 | — | — | 730497 | NM_032449.1 | NM_032449 | coiled-coil and C2 | CC2D1B | RP11- | GGAGATTAGT | P |
| | | | | | | domain containing 1B | | 155O18.2; | GACTCACCTG | |
| | | | | | | (CC2D1B), mRNA. | | KIAA1836 | CAGTTGGGA | |
| | | | | | | | | | GCCAGCTAC | |
| | | | | | | | | | AACCCAAATC | |
| | | | | | | | | | AT | |
| 1641 | — | — | 4220220 | NM_001009922.1 | NM_001009922 | ring finger and CHY | RCHY1 | ARNIP; | CTTCCAAGG | N |
| | | | | | | zinc finger domain | | PRO1996; | GCTAGGATTA | |
| | | | | | | containing 1 (RCHY1), | | CHIMP; | CAGGCATGA | |
| | | | | | | transcript variant 3, | | DKFZp586C1620; | GCCACTGTG | |
| | | | | | | mRNA. | | ZNF363; | CTTGGTCCAG | |
| | | | | | | | | hARNIP; | ATG | |
| | | | | | | | | PIRH2; | | |
| | | | | | | | | RNF199 | | |
| 1642 | — | — | 1990653 | NM_006405.5 | NM_006405 | transmembrane 9 | TM9SF1 | HMP70; | GGGCCATTAA | P |
| | | | | | | superfamily member 1 | | MP70 | CTCAGCAGC | |
| | | | | | | (TM9SF1), transcript | | | CATCTTGTTG | |
| | | | | | | variant 1, mRNA. | | | TATGCCCTGA | |
| | | | | | | | | | CCTGCTGCATC | |
| 1643 | — | — | 5490717 | NM_198585.2 | NM_198585 | ectonucleoside | ENTPD8 | GLSR2492; | AGGGCCACA | N |
| | | | | | | triphosphate | | UNQ2492; | TGCTGCCTG | |
| | | | | | | diphosphohydrolase 8 | | NTPDase-8 | CAAACAGGG | |
| | | | | | | (ENTPD8), transcript | | | CAAGACCAC | |
| | | | | | | variant 2, mRNA. | | | GGAGGCACA | |
| | | | | | | | | | GGGGT | |
| 1644 | — | — | 1010739 | NM_033364.3 | NM_033364 | chromosome 3 open | C3ORF15 | AAT1alpha; | CTGAAGGGC | P |
| | | | | | | reading frame 15 | | AAT1; | CAGATGGTAA | |
| | | | | | | (C3orf15), mRNA. | | DKFZp781A2221 | CTACATTAGG | |
| | | | | | | | | | GTTTGCGGG | |
| | | | | | | | | | TCTGATGGTC | |
| | | | | | | | | | GC | |
| 1645 | — | — | 1340681 | NM_015633.1 | NM_015633 | FGFR1 oncogene | FGFR1OP2 | DKFZp564O1863; | AGCTGAAAGT | N |
| | | | | | | partner 2 | | HSPC123- | GGGGGTAAA | |
| | | | | | | (FGFR1OP2), mRNA. | | like | GGTGGAGTA | |
| | | | | | | | | | ATCTGTGGAT | |
| | | | | | | | | | TTGTTTCTGT | |
| | | | | | | | | | TG | |
| 1646 | — | — | 2680497 | NM_014865.2 | NM_014865 | non-SMCcondensin I | NCAPD2 | hCAP-D2; | GATCCTAGGA | P |
| | | | | | | complex, subunit D2 | | KIAA0159; | AGTCTGTTCC | |
| | | | | | | (NCAPD2), mRNA. | | CAP-D2; | TGTCCTCCCT | |
| | | | | | | | | CNAP1 | GTGCAGGGT | |
| | | | | | | | | | ATCCTGTAGGG | |
| 1647 | — | — | 830440 | NM_003268.4 | NM_003268 | toll-like receptor 5 | TLR5 | MGC126430; | TGCCCAGGG | N |
| | | | | | | (TLR5), mRNA. | | SLEB1; | CAGGTGCTTA | |
| | | | | | | | | MGC126431; | TCTGACCTTA | |
| | | | | | | | | FLJ10052; | ACAGTGCTCT | |
| | | | | | | | | TIL3 | CATCATGGTGG | |
| 1648 | 32 | 272 | 2810082 | NM_016470.6 | NM_016470 | chromosome 20 open | C20ORF111 | dJ1183I21.1; | GAGTCTTCGT | P |
| | | | | | | reading frame 111 | | HSPC207; | GGATGATGT | |
| | | | | | | (C20orf111), mRNA. | | Perit1 | GACCATTGAG | |
| | | | | | | | | | GACCTGTCA | |
| | | | | | | | | | GGCTACATG | |
| | | | | | | | | | GAG | |
| 1649 | — | — | 3800253 | NM_172388.1 | NM_172388 | nuclear factor of | NFATC1 | NFATc; | GAAAGGAGA | N |
| | | | | | | activated T-cells, | | MGC138448; | GACGGACAT | |
| | | | | | | cytoplasmic, | | NFAT2; | CGGGAGGAA | |
| | | | | | | calcineurin-dependent | | NF-ATC | GAACACACG | |
| | | | | | | 1 (NFATC1), transcript | | | GGTACGGCT | |
| | | | | | | variant 4, mRNA. | | | GGTGT | |
| 1650 | — | — | 2000669 | NM_024605.3 | NM_024605 | Rho GTPase activating | ARHGAP10 | FLJ20896; | GCTGTTGGT | P |
| | | | | | | protein 10 | | PS-GAP; | GCAAGGGAG | |
| | | | | | | (ARHGAP10), mRNA. | | GRAF2; | ATGGTCTCAA | |
| | | | | | | | | FLJ41791 | GTCAGAGGG | |
| | | | | | | | | | AAGCAGAGA | |
| | | | | | | | | | CGCG | |
| 1651 | — | — | 6290181 | NM_058192.2 | NM_058192 | RNA pseudouridylate | RPUSD1 | MGC19600; | TGGCTCCCA | N |
| | | | | | | synthase domain | | RLUCL; | CACAGCCAT | |
| | | | | | | containing 1 | | C16orf40 | GCATTGTCAC | |
| | | | | | | (RPUSD1), mRNA. | | | TCTGCCTCCG | |
| | | | | | | | | | GGACCCCAG | |
| | | | | | | | | | CTT | |
| 1652 | 33 | 1084 | 5690333 | NM_003400.3 | NM_003400 | exportin 1 (CRM1 | XPO1 | DKFZp686B1823; | GTGCTGCATT | P |
| | | | | | | homolog, yeast) | | CRM1 | GTCTGAAGTT | |
| | | | | | | (XPO1), mRNA. | | | AGCACCTCTT | |
| | | | | | | | | | GGACTGAATC | |
| | | | | | | | | | GTTTGTCTAG | |
| 1653 | — | — | 7050291 | NM_024756.1 | NM_024756 | multimerin 2 (MMRN2), | MMRN2 | EndoGlyx- | CCCACATCAA | N |
| | | | | | | mRNA. | | 1; | GGGAGAGAT | |
| | | | | | | | | FLJ13465; | CAGGTGGAG | |
| | | | | | | | | EMILIN3; | GTAATTGGAT | |
| | | | | | | | | ENDOGLYX1 | CTTGGGGGC | |
| | | | | | | | | | GGT | |
| 1654 | — | — | 3370280 | NM_016447.2 | NM_016447 | membrane protein, | MPP6 | p55T; VAM- | CTGTGGTGG | P |
| | | | | | | palmitoylated 6 | | 1; VAM1; | ATGCAGGAAT | |
| | | | | | | (MAGUK p55 | | PALS2 | CACTACCAAG | |
| | | | | | | subfamily member 6) | | | CTTCTGACCG | |
| | | | | | | (MPP6), mRNA. | | | ACTCTGACTTG | |
| 1655 | — | — | 1050040 | NM_004925.3 | NM_004925 | aquaporin 3 (Gill blood | AQP3 | GIL | GCTTCTACAG | N |
| | | | | | | group) (AQP3), mRNA. | | | GCTTTTGGGA | |
| | | | | | | | | | AGTAGGGTG | |
| | | | | | | | | | GATGTGGGT | |
| | | | | | | | | | AGGGCTGGG | |
| | | | | | | | | | AGG | |
| 1656 | — | — | 6580379 | NM_006348.2 | NM_006348 | component of | COG5 | GOLTC1; | GTTGAGGAA | P |
| | | | | | | oligomericgolgi | | GTC90 | CCACTGGCA | |
| | | | | | | complex 5 (COG5), | | | CATCCCAAGC | |
| | | | | | | transcript variant 1, | | | TAAGATACAA | |
| | | | | | | mRNA. | | | GGTTAAATGG | |
| | | | | | | | | | CC | |
| 1657 | — | — | 730487 | NM_020320.2 | NM_020320 | arginyl- | RARS2 | dJ382I10.6; | AGAGTCGCG | N |
| | | | | | | tRNAsynthetase 2, | | DALRD2; | GGGACACAG | |
| | | | | | | mitochondrial | | MGC14993; | GAGTCTTCCT | |
| | | | | | | (RARS2), nuclear gene | | PCH6; | ACAGTACACA | |
| | | | | | | encoding mitochondrial | | PRO1992; | CACGCCCGC | |
| | | | | | | protein, mRNA. | | RARSL; | CTC | |
| | | | | | | | | MGC23778 | | |
| 1658 | — | — | 2140682 | NM_175617.2 | NM_175617 | metallothionein 1E | MT1E | MT1; MTD | CGTGGGACA | P |
| | | | | | | (functional) (MT1E), | | | CAAACCCCAA | |
| | | | | | | mRNA. | | | CTGTACCCCC | |
| | | | | | | | | | TATGGTTTCA | |
| | | | | | | | | | GAACAGAGC | |
| | | | | | | | | | TG | |
| 1659 | — | — | 2650148 | NM_018268.2 | NM_018268 | WD repeat domain 41 | WDR41 | MSTP048; | GCTGAGCGC | N |
| | | | | | | (WDR41), mRNA. | | FLJ10904 | AACTGCCCCA | |
| | | | | | | | | | TCTGACCACT | |
| | | | | | | | | | GACTCAAATA | |
| | | | | | | | | | CGAACTGCTTG | |
| 1660 | 34 | 1316 | 7000735 | NM_002882.2 | NM_002882 | RAN binding protein 1 | RANBP1 | MGC88701 | CTGTTCCGAT | P |
| | | | | | | (RANBP1), mRNA. | | | TTGCCTCTGA | |
| | | | | | | | | | GAACGATCTC | |
| | | | | | | | | | CCAGAATGG | |
| | | | | | | | | | AAGGAGCGA | |
| | | | | | | | | | GG | |
| 1661 | — | — | 3130471 | NM_198465.2 | NM_198465 | Nik related kinase | NRK | DKFZp686A17109; | CAGAAGTGT | N |
| | | | | | | (NRK), mRNA. | | FLJ16788; | GGAGGGGGG | |
| | | | | | | | | NESK; | CTCCTGACTA | |
| | | | | | | | | MGC131849 | GACAATTTCC | |
| | | | | | | | | | CTAGCCCTTG | |
| | | | | | | | | | TG | |
| 1662 | — | — | 4810274 | NM_199367.1 | NM_199367 | spastic paraplegia 7 | SPG7 | CAR; | TCCCTTCAAC | P |
| | | | | | | (pure and complicated | | FLJ37308; | GTAGTCATCC | |
| | | | | | | autosomal recessive) | | SPG5C; | CCTGGTGGT | |
| | | | | | | (SPG7), nuclear gene | | MGC126332; | GGAAGCAAG | |
| | | | | | | encoding mitochondrial | | CMAR; | ACGACGGCC | |
| | | | | | | protein, transcript | | MGC126331; | CCT | |
| | | | | | | variant 2, mRNA. | | PGN | | |
| 1663 | 35 | 1369 | 7510687 | NM_006662.2 | NM_006662 | Snf2-related CREBBP | SRCAP | EAF1; | CTAGTCCCCC | N |
| | | | | | | activator protein | | SWR1; | CACTAGAGAC | |
| | | | | | | (SRCAP), mRNA. | | DOMO1; | TGAGAAGTTG | |
| | | | | | | | | KIAA0309; | CCTCGCAAAC | |
| | | | | | | | | FLJ44499 | GAGCAGGGGC | |
| 1664 | 36 | 1085 | 5690358 | NM_014254.1 | NM_014254 | transmembrane protein | TMEM5 | HP10481 | GAGGCTTGC | P |
| | | | | | | 5 (TMEM5), mRNA. | | | TCCTATGGCT | |
| | | | | | | | | | CCATTCCTGT | |
| | | | | | | | | | GGTGGAAGA | |
| | | | | | | | | | CGTGATGACA | |
| | | | | | | | | | GC | |
| 1665 | — | — | 5670100 | NM_000355.2 | NM_000355 | transcobalamin II; | TCN2 | D22S750; | CTGCAGGTCT | N |
| | | | | | | macrocytic anemia | | TC2; | CCCATGAAG | |
| | | | | | | (TCN2), mRNA. | | D22S676 | GCCACCCCA | |
| | | | | | | | | | TGGTCTGATG | |
| | | | | | | | | | GGCATGAAG | |
| | | | | | | | | | CAT | |
| 1666 | 37 | 1187 | 6270020 | NM_145799.2 | NM_145799 | septin 6 (SEPT6), | SEPT6 | SEP2; | GATGGAGTT | P |
| | | | | | | transcript variant I, | | RP5- | GACCTGGCA | |
| | | | | | | mRNA. | | 876A24.2; | ATGATCTGTG | |
| | | | | | | | | MGC16619; | GCTAACATGC | |
| | | | | | | | | SEPT2; | CGTCTCTCTG | |
| | | | | | | | | MGC20339; | CC | |
| | | | | | | | | KIAA0128 | | |
| 1667 | — | — | 7320441 | NM_013332.3 | NM_013332 | hypoxia-inducible | HIG2 | FLJ21076; | GTCGTTCCTC | N |
| | | | | | | protein 2 (HIG2), | | MGC138388 | CAACATAGTG | |
| | | | | | | transcript variant 1, | | | TGTATTGGTC | |
| | | | | | | mRNA. | | | TGAAGGGGG | |
| | | | | | | | | | TGGTGGGAT | |
| | | | | | | | | | GC | |
| 1668 | 38 | 59 | 1170332 | NM_014911.3 | NM_014911 | AP2 associated kinase | AAK1 | DKFZp686K16132; | GAGCACCTT | P |
| | | | | | | 1 (AAK1), mRNA. | | MGC164568; | GTTACAGTTC | |
| | | | | | | | | FLJ45252; | CGGCCTCTC | |
| | | | | | | | | FLJ23712; | AGTATGTGG | |
| | | | | | | | | FLJ25931; | GCTAAATGCC | |
| | | | | | | | | KIAA1048; | AGC | |
| | | | | | | | | FLJ42882; | | |
| | | | | | | | | DKFZp686F03202; | | |
| | | | | | | | | MGC164570; | | |
| | | | | | | | | FLJ31060; | | |
| | | | | | | | | MGC138170 | | |
| 1669 | — | — | 2060674 | NM_000067.1 | NM_000067 | carbonic anhydrase II | CA2 | Car2; CA-II; | AGTACCTTGA | N |
| | | | | | | (CA2), mRNA. | | CAII; CA II | CTTTGTTCAC | |
| | | | | | | | | | AGCATGTAG | |
| | | | | | | | | | GGTGATGAG | |
| | | | | | | | | | CACTCACAAT | |
| | | | | | | | | | TG | |
| 1670 | — | — | 6350671 | NM_023080.1 | NM_023080 | chromosome 8 open | C8ORF33 | FLJ20989 | GGCTTGGTCT | P |
| | | | | | | reading frame 33 | | | AGCAGTAACA | |
| | | | | | | (C8orf33), mRNA. | | | CCAGTGTCTG | |
| | | | | | | | | | GGAAGATGC | |
| | | | | | | | | | CTGTTGCAAAG | |
| 1671 | — | — | 770619 | NM_003473.2 | NM_003473 | signal transducing | STAM | DKFZp686J2352; | TCTGTAGCCT | N |
| | | | | | | adaptor molecule (SH3 | | STAM1 | CTGCATACTA | |
| | | | | | | domain and ITAM | | | CTGGCTGTCA | |
| | | | | | | motif) 1 (STAM), | | | TCACACCAGC | |
| | | | | | | mRNA. | | | GTACAGTAGC | |
| 1672 | — | — | 540452 | NM_022743.1 | NM_022743 | SET and MYND | SMYD3 | bA74P14.1; | GAAGAATGC | P |
| | | | | | | domain containing 3 | | ZMYND1; | GACGCCAAC | |
| | | | | | | (SMYD3), mRNA. | | ZNFN3A1; | ATCAGAGCAT | |
| | | | | | | | | FLJ21080; | CCTAAGGGA | |
| | | | | | | | | MGC104324 | ACGCAGTCA | |
| | | | | | | | | | GAGG | |
| 1673 | — | — | 4200441 | NM_003003.2 | NM_003003 | SEC14-like 1 (S. cerevisiae) | SEC14L1 | SEC14L; | CGCCCACCC | N |
| | | | | | | (SEC14L1), | | DKFZp686C06176; | AGCGGCGAC | |
| | | | | | | transcript variant 1, | | PRELID4A | ATTGTACAGA | |
| | | | | | | mRNA. | | | CTCCTCTCAC | |
| | | | | | | | | | CTCTAGATAG | |
| | | | | | | | | | CA | |
| 1674 | — | — | 6550279 | NM_000848.2 | NM_000848 | glutathione S- | GSTM2 | GST4; | ATGTCCTTGA | P |
| | | | | | | transferase M2 | | GSTM; | GAGAAACCAA | |
| | | | | | | (muscle) (GSTM2), | | GSTM2-2; | GTATTTGAGC | |
| | | | | | | mRNA. | | GTHMUS; | CCAGCTGCC | |
| | | | | | | | | MGC117303 | TGGATGCCTTC | |
| 1675 | — | — | 4860392 | NM_178128.3 | NM_178128 | fatty acid desaturase | FADS6 | FP18279 | GTGTGTTTTG | N |
| | | | | | | domain family, | | | TCGGGAGGG | |
| | | | | | | member 6 (FADS6), | | | AACTCCAGG | |
| | | | | | | mRNA. | | | GGAAGTGAG | |
| | | | | | | | | | GGGAGAAGG | |
| | | | | | | | | | TTCC | |
| 1676 | — | — | 3940735 | NM_003093.1 | NM_003093 | small nuclear | SNRPC | FLJ20302 | CGGCTGCATT | P |
| | | | | | | ribonucleoprotein | | | TCAACAAGGA | |
| | | | | | | polypeptide C | | | AAGATACCTC | |
| | | | | | | (SNRPC), mRNA. | | | CTACTCCATT | |
| | | | | | | | | | CTCTGCTCCT | |
| 1677 | — | — | 1850347 | NM_021067.3 | NM_021067 | GINS complex subunit | GINS1 | PSF1; | CCAGTATCAC | N |
| | | | | | | 1 (Psf1 homolog) | | KIAA0186; | CACTTTGGAA | |
| | | | | | | (GINS1), mRNA. | | RP4- | GGGGACAGT | |
| | | | | | | | | 691N24.2 | GAAATTGGG | |
| | | | | | | | | | GCTAGAGAA | |
| | | | | | | | | | GGA | |
| 1678 | 39 | 736 | 2750184 | NM_005184.2 | NM_005184 | calmodulin 3 | CALM3 | PHKD; | CAGCCAAGA | P |
| | | | | | | (phosphorylase kinase, | | PHKD3 | GCTGAGGGT | |
| | | | | | | delta) (CALM3), | | | AAGGGCAGG | |
| | | | | | | mRNA. | | | TAGGCGTGA | |
| | | | | | | | | | GGCTGTGGA | |
| | | | | | | | | | CATTT | |
| 1679 | — | — | 7000703 | NM_016310.2 | NM_016310 | polymerase (RNA) III | POLR3K | C11; | CCTTTTGAGG | N |
| | | | | | | (DNA directed) | | RPC10; | TGAAGAGCC | |
| | | | | | | polypeptide K, 12.3 kDa | | C11-RNP3; | AGGGGGTCA | |
| | | | | | | (POLR3K), | | My010; | GGAAATATGG | |
| | | | | | | mRNA. | | RPC11; | CCTATCTGCC | |
| | | | | | | | | hRPC11 | AG | |
| 1680 | — | — | 2690315 | NM_014901.4 | NM_014901 | ring finger protein 44 | RNF44 | KIAA1100 | CCCAGCCCT | P |
| | | | | | | (RNF44), mRNA. | | | GGCTGGGCC | |
| | | | | | | | | | CAGCGCCTG | |
| | | | | | | | | | TGTTCTGTGT | |
| | | | | | | | | | TAGAAAGGTT | |
| | | | | | | | | | TTA | |
| 1681 | — | — | 4900670 | NM_004255.2 | NM_004255 | cytochrome c oxidase | COX5A | COX-VA; | AACTGGGCC | N |
| | | | | | | subunit Va (COX5A), | | COX | TTGACAAAGT | |
| | | | | | | nuclear gene encoding | | VA; | GTAAACCGCA | |
| | | | | | | mitochondrial protein, | | | TGGATGGGC | |
| | | | | | | mRNA. | | | TTCCCCAAGG | |
| | | | | | | | | | AT | |
| 1682 | — | — | 6900014 | NM_032177.2 | NM_032177 | RNA U, small nuclear | RNUXA | FLJ13193; | GGCAATTTTA | P |
| | | | | | | RNA export adaptor | | PHAX | AGGATAAAAA | |
| | | | | | | (phosphorylation | | | CTAACATTGG | |
| | | | | | | regulated) (RNUXA), | | | CCAGGCACG | |
| | | | | | | mRNA. | | | GTGGCTCAC | |
| | | | | | | | | | GC | |
| 1683 | 40 | 1078 | 5570601 | NM_020216.3 | NM_020216 | arginylaminopeptidase | RNPEP | DKFZP547H084 | CACTGCAGG | N |
| | | | | | | (aminopeptidase B) | | | GCAGCGGGT | |
| | | | | | | (RNPEP), mRNA. | | | ATTCTCCTCC | |
| | | | | | | | | | CCACCTAAGT | |
| | | | | | | | | | CTCTGGGAA | |
| | | | | | | | | | GAA | |
| 1684 | — | — | 5570338 | NM_182922.2 | NM_182922 | HEAT repeat | HEATR3 | FLJ20718 | TCTGTACATT | P |
| | | | | | | containing 3 | | | CTGTAAAAAC | |
| | | | | | | (HEATR3), mRNA. | | | TTCAAAACCT | |
| | | | | | | | | | GGCCAGGCA | |
| | | | | | | | | | TGGTGGCTC | |
| | | | | | | | | | AC | |
| 1685 | — | — | 4540241 | NM_032412.3 | NM_032412 | chromosome 5 open | C5ORF32 | ORF1-FL49 | GCCACCTCT | N |
| | | | | | | reading frame 32 | | | GACAGGTGT | |
| | | | | | | (C5orf32), mRNA. | | | GCCTGCCCC | |
| | | | | | | | | | CATCTCTTCT | |
| | | | | | | | | | GATTGCTGTT | |
| | | | | | | | | | AAC | |
| 1686 | — | — | 2850360 | NM_001707.2 | NM_001707 | B-cell CLL/lymphoma | BCL7B | | TCTGGACGG | P |
| | | | | | | 7B (BCL7B), mRNA. | | | AGCTGCTGG | |
| | | | | | | | | | CAGCTTCTGC | |
| | | | | | | | | | GAGAAGAGA | |
| | | | | | | | | | GAGATGTGG | |
| | | | | | | | | | AAGG | |
| 1687 | — | — | 6860653 | NM_006402.2 | NM_006402 | hepatitis B virus x | HBXIP | MGC71071; | ATGATCCAGA | N |
| | | | | | | interacting protein | | XIP | AACACGATG | |
| | | | | | | (HBXIP), mRNA. | | | GCATCACGG | |
| | | | | | | | | | TGGCAGTGC | |
| | | | | | | | | | ACAAAATGGC | |
| | | | | | | | | | CTC | |
| 1688 | — | — | 2350209 | NM_139118.1 | NM_139118 | YY1 associated protein | YY1AP1 | YAP; | TGCAACTGG | P |
| | | | | | | 1 (YY1AP1), transcript | | YY1AP; | GGCTCTTGA | |
| | | | | | | variant 2, mRNA. | | HCCA2; | GCAGCTTGCT | |
| | | | | | | | | FLJ10875; | TTAGCCTGCT | |
| | | | | | | | | FLJ13914; | CCCACTCTGT | |
| | | | | | | | | HCCA1 | GG | |
| 1689 | — | — | 3940754 | NM_006566.1 | NM_006566 | CD226 molecule | CD226 | TLiSA1; | CTTGCCGCC | N |
| | | | | | | (CD226), mRNA. | | PTA1; | ATCCCAGGTC | |
| | | | | | | | | DNAM1; | TAGCCTTAGG | |
| | | | | | | | | DNAM-1 | AGCAAATGTA | |
| | | | | | | | | | GTAGATAGTCG | |
| 1690 | — | — | 6650747 | NM_152320.1 | NM_152320 | zinc finger protein 641 | ZNF641 | FLJ31295; | AGCCAGGGG | P |
| | | | | | | (ZNF641), mRNA. | | DKFZp667D1012 | GGCCAGACC | |
| | | | | | | | | | TTGTTCATGT | |
| | | | | | | | | | GTGGGTCTG | |
| | | | | | | | | | TCTTCCTTAT | |
| | | | | | | | | | GCC | |
| 1691 | — | — | 3780400 | NM_014212.3 | NM_014212 | homeobox C11 | HOXC11 | HOX3H; | TTGAAGATTG | N |
| | | | | | | (HOXC11), mRNA. | | MGC4906 | GGGTGGTGG | |
| | | | | | | | | | AGGCAGTAG | |
| | | | | | | | | | GGAGATGGG | |
| | | | | | | | | | ATTGGGCAC | |
| | | | | | | | | | CTCC | |
| 1692 | — | — | 6770017 | NM_007249.4 | NM_007249 | Kruppel-like factor 12 | KLF12 | AP-2rep; | CCATGGCAAA | P |
| | | | | | | (KLF12), mRNA. | | AP2REP; | GCACAAATG | |
| | | | | | | | | HSPC122 | GACCCCCGA | |
| | | | | | | | | | GGCCTATCTC | |
| | | | | | | | | | CCAGACAAA | |
| | | | | | | | | | GTA | |
| 1693 | — | — | 2340059 | NM_024516.2 | NM_024516 | chromosome 16 open | C16ORF53 | PA1; | ACCTCAAGCT | N |
| | | | | | | reading frame 53 | | MGC4606 | CCCAAACAG | |
| | | | | | | (C16orf53), mRNA. | | | CACGTTGCG | |
| | | | | | | | | | GGAAAGAGG | |
| | | | | | | | | | AAGAGAGAG | |
| | | | | | | | | | TGTG | |
| 1694 | — | — | 4150593 | NM_015077.2 | NM_015077 | sterile alpha and TIR | SARM1 | SAMD2; | AGCTGTGTGA | P |
| | | | | | | motif containing 1 | | KIAA0524; | CCGGGAGTA | |
| | | | | | | (SARM1), mRNA. | | SARM; | GTCACTTAAC | |
| | | | | | | | | FLJ36296 | CTATGTCTCC | |
| | | | | | | | | | CCTTCCTCACC | |
| 1695 | — | — | 2650408 | NM_018177.2 | NM_018177 | Nedd4 binding protein | N4BP2 | B3BP; | GAGGTATTTA | N |
| | | | | | | 2 (N4BP2), mRNA. | | KIAA1413; | AAGTGCTTTG | |
| | | | | | | | | FLJ10680 | AGACCTGATT | |
| | | | | | | | | | CATGCCCCC | |
| | | | | | | | | | CAAAGGGTG | |
| | | | | | | | | | GT | |
| 1696 | — | — | 10435 | NM_001001660.2 | NM_001001660 | LYR motif containing 5 | LYRM5 | | ATGTATGGAG | P |
| | | | | | | (LYRM5), mRNA. | | | TCATTACTTC | |
| | | | | | | | | | TGACCTTGAA | |
| | | | | | | | | | ATAGCCTGCT | |
| | | | | | | | | | GGTGACTGGC | |
| 1697 | — | — | 2690528 | NM_004169.3 | NM_004169 | serinehydroxymethyltransferase | SHMT1 | MGC15229; | CCAGGCTTTC | N |
| | | | | | | 1 (soluble) | | MGC24556; | CTGCTCCACC | |
| | | | | | | (SHMT1), transcript | | SHMT; | TGAGATAACC | |
| | | | | | | variant 1, mRNA. | | CSHMT | AACTCCCTCC | |
| | | | | | | | | | CGTAATCAGG | |
| 1698 | — | — | 130093 | NM_005951.2 | NM_005951 | metallothionein 1H | MT1H | MGC70702; | CTTCTCGCTT | P |
| | | | | | | (MT1H), mRNA. | | MT1 | GGGAACTCC | |
| | | | | | | | | | AGTCTCACCT | |
| | | | | | | | | | CGGCTTGCA | |
| | | | | | | | | | ATGGACCCC | |
| | | | | | | | | | AAC | |
| 1699 | — | — | 1010692 | NM_005234.3 | NM_005234 | nuclear receptor | NR2F6 | EAR-2; | CCCCTAGCAT | N |
| | | | | | | subfamily 2, group F, | | EAR2; | GAACTTGTGG | |
| | | | | | | member 6 (NR2F6), | | ERBAL2 | GATGGTGGG | |
| | | | | | | mRNA. | | | GTTGGCTTCC | |
| | | | | | | | | | CTGGCATGATG | |
| 1700 | 41 | 374 | 360280 | NM_017761.2 | NM_017761 | proline-rich nuclear | PNRC2 | MGC99541; | GTTTGGTCAA | P |
| | | | | | | receptor coactivator 2 | | FLJ20312 | GGGGTAGGT | |
| | | | | | | (PNRC2), mRNA. | | | GCAACCCAAT | |
| | | | | | | | | | GGACCACTTA | |
| | | | | | | | | | TGCAAAAGATG | |
| 1701 | 42 | 1281 | 6760347 | NM_178009.2 | NM_178009 | diacylglycerol kinase, | DGKH | DKFZp761I1510; | ATGGGGCAC | N |
| | | | | | | eta (DGKH), transcript | | DGKeta | AGAGGAAGTT | |
| | | | | | | variant 2, mRNA. | | | GCTGCTTGG | |
| | | | | | | | | | CTGGATCTGC | |
| | | | | | | | | | TCAATTTGGG | |
| | | | | | | | | | AG | |
| 1702 | — | — | 520431 | NM_014819.3 | NM_014819 | praja 2, RING-H2 motif | PJA2 | KIAA0438, | AGCCCAGGT | P |
| | | | | | | containing (PJA2), | | RNF131; | CTAAATGTAA | |
| | | | | | | mRNA. | | Neurodap1 | TGGTTGGTTT | |
| | | | | | | | | | ATTGTTCTAT | |
| | | | | | | | | | AACCCCAGC | |
| | | | | | | | | | CC | |
| 1703 | — | — | 5560079 | NM_001077191.1 | NM_001077191 | G protein-coupled bile | GPBAR1 | GPR131; | CTGGATCAGA | N |
| | | | | | | acid receptor 1 | | M-BAR; | GACCCTGCC | |
| | | | | | | (GPBAR1), transcript | | GPCR; | TCTGTTTGAC | |
| | | | | | | variant 1, mRNA. | | GPCR19; | CCCGCACTG | |
| | | | | | | | | BG37; | ACTGAATAAA | |
| | | | | | | | | TGR5; | GC | |
| | | | | | | | | MGC40597 | | |
| 1704 | — | — | 670026 | NM_015986.2 | NM_015986 | cytokine receptor-like | CRLF3 | CREME9; | GCACGTGTG | P |
| | | | | | | factor 3 (CRLF3), | | FRWS; | TATCCAATCT | |
| | | | | | | mRNA. | | CYTOR4; | GCCTGTGAC | |
| | | | | | | | | MGC20661 | ATGCATTTTA | |
| | | | | | | | | | CTCTTTGCAG | |
| | | | | | | | | | AG | |
| 1705 | 43 | 160 | 4230619 | NM_012198.2 | NM_012198 | grancalcin, EF-hand | GCA | GCL | GTTGGTGGT | N |
| | | | | | | calcium binding protein | | | GTTTGAGGGT | |
| | | | | | | (GCA), mRNA. | | | TGGCTAGAAA | |
| | | | | | | | | | TGAAAGCCTG | |
| | | | | | | | | | GATTTTGTGCC | |
| 1706 | 44 | 459 | 830463 | NM_002735.1 | NM_002735 | protein kinase, cAMP- | PRKAR1B | PRKAR1 | GGCCAAGGC | P |
| | | | | | | dependent, regulatory, | | | CATCTCCAAG | |
| | | | | | | type I, beta | | | AACGTGCTCT | |
| | | | | | | (PRKAR1B), mRNA. | | | TCGCTCACCT | |
| | | | | | | | | | GGATGACAA | |
| | | | | | | | | | CG | |
| 1707 | 45 | 353 | 130364 | NM_032947.3 | NM_032947 | MSTP150 (MST150), | MST150 | NID67; | CTGTAATTAG | N |
| | | | | | | mRNA. | | MGC126887; | CTCCACGTGT | |
| | | | | | | | | MGC126889; | ACCCCCTTCA | |
| | | | | | | | | MGC117221 | CTCCCTCCCA | |
| | | | | | | | | | CCAGCTCTGC | |
| 1708 | 46 | 52 | 1050408 | NM_005678.3 | NM_005678 | SNRPN upstream | SNURF | | AGTGGAGCG | P |
| | | | | | | reading frame | | | GCCGCCGGA | |
| | | | | | | (SNURF), transcript | | | GATGCCTGA | |
| | | | | | | variant 1, mRNA. | | | CGCATCTGTC | |
| | | | | | | | | | TGAGGAGCG | |
| | | | | | | | | | GTCA | |
| 1709 | — | — | 1770593 | NM_003956.3 | NM_003956 | cholesterol 25- | CH25H | C25H | CGGTGGGTG | N |
| | | | | | | hydroxylase (CH25H), | | | CCCCTAAGAC | |
| | | | | | | mRNA. | | | TCGGGACTG | |
| | | | | | | | | | CTGTGCCTTT | |
| | | | | | | | | | CACACTTGAA | |
| | | | | | | | | | TG | |
| 1710 | 47 | 58 | 1170300 | NM_005950.1 | NM_005950 | metallothionein 1G | MT1G | MT1; | CGCCTGATGT | P |
| | | | | | | (MT1G), mRNA. | | MT1K; | CGGGACAGC | |
| | | | | | | | | MGC12386 | CCTGCTCCCA | |
| | | | | | | | | | AGTACAAATA | |
| | | | | | | | | | GAGTGACCC | |
| | | | | | | | | | GT | |
| 1711 | 48 | 23 | 20056 | NM_003295.1 | NM_003295 | tumor protein, | TPT1 | TCTP; p02; | CCAGATGGC | N |
| | | | | | | translationally- | | HRF; | ATGGTTGCTC | |
| | | | | | | controlled 1 (TPT1), | | FLJ27337 | TATTGGACTA | |
| | | | | | | mRNA. | | | CCGTGAGGA | |
| | | | | | | | | | TGGTGTGAC | |
| | | | | | | | | | CCC | |
| 1712 | — | — | 1260438 | NM_001556.1 | NM_001556 | inhibitor of kappa light | IKBKB | IKK-beta; | GTGCTGGGC | P |
| | | | | | | polypeptide gene | | NFKBIKB; | CGGGGAGTC | |
| | | | | | | enhancer in B-cells, | | IKK2; | CCTGTCTCTC | |
| | | | | | | kinase beta (IKBKB), | | FLJ40509; | ACAGCATCTA | |
| | | | | | | mRNA. | | IKKB; | GCAGTATTAT | |
| | | | | | | | | MGC131801 | TA | |
| 1713 | — | — | 4050768 | NM_152889.1 | NM_152889 | carbohydrate | CHST13 | MGC119279; | CAGAGCCCC | N |
| | | | | | | (chondroitin 4) | | MGC119281; | TGGTGCAATG | |
| | | | | | | sulfotransferase 13 | | MGC119278; | CGGTCACAG | |
| | | | | | | (CHST13), mRNA. | | C4ST3 | GTTTTATGGG | |
| | | | | | | | | | ACTTTGGTGA | |
| | | | | | | | | | GC | |
| 1714 | — | — | 1340349 | NM_001042588.1 | NM_001042588 | snurportin 1 (SNUPN), | SNUPN | RNUT1; | CCCAAGTTGA | P |
| | | | | | | transcript variant 3, | | Snurportin1; | AGGGTTCTTC | |
| | | | | | | mRNA. | | KPNBL | CCATAGCCCA | |
| | | | | | | | | | GACCACCCT | |
| | | | | | | | | | GGATGCCTC | |
| | | | | | | | | | AT | |
| 1715 | — | — | 6940431 | NM_015253.1 | NM_015253 | WSC domain | WSCD1 | KIAA0523 | CCTGGACTAA | N |
| | | | | | | containing 1 (WSCD1), | | | GCCAATGACA | |
| | | | | | | mRNA. | | | CCTTCCATCT | |
| | | | | | | | | | TTCCAGCTAT | |
| | | | | | | | | | GGTGACTGGG | |
| 1716 | — | — | 3610241 | NM_000981.3 | NM_000981 | ribosomal protein L19 | RPL19 | MGC71997; | ATGGGCATA | HSK |
| | | | | | | (RPL19), mRNA. | | DKFZp779D216; | GGTAAGCGG | |
| | | | | | | | | FLJ27452 | AAGGGTACA | |
| | | | | | | | | | GCCAATGCC | |
| | | | | | | | | | CGAATGCCA | |
| | | | | | | | | | GAGAA | |
| 1717 | — | — | 6580577 | NM_031369.2 | NM_031369 | heterogeneous nuclear | HNRNPD | P37; AUF1; | CCCCCAGTAT | HSK |
| | | | | | | ribonucleoprotein D | | hnRNPD0; | TGTAGAGCAA | |
| | | | | | | (AU-rich element RNA | | AUF1A | GTCTTGTGTT | |
| | | | | | | binding protein 1, | | | AAAAGCCCA | |
| | | | | | | 37 kDa) (HNRNPD), | | | GTGTGACAGTG | |
| | | | | | | transcript variant 2, | | | | |
| | | | | | | mRNA. | | | | |
| 1718 | — | — | 3360228 | NM_001023.2 | NM_001023 | ribosomal protein S20 | RPS20 | MGC102930; | CGCGCGCAA | HSK |
| | | | | | | (RPS20), mRNA. | | FLJ27451 | CAGCCATGG | |
| | | | | | | | | | CTTTTAAGGA | |
| | | | | | | | | | TACCGGAAAA | |
| | | | | | | | | | ACACCCGTG | |
| | | | | | | | | | GAG | |
| 1719 | — | — | 6130390 | NM_016093.2 | NM_016093 | ribosomal protein L26- | RPL26L1 | RPL26P1; | TCATCTACAT | HSK |
| | | | | | | like 1 (RPL26L1), | | FLJ46904 | CGAGCGGGT | |
| | | | | | | mRNA. | | | GCAGCGTGA | |
| | | | | | | | | | GAAGGCCAA | |
| | | | | | | | | | CGGCACAAC | |
| | | | | | | | | | TGTC | |
| 1720 | 49 | 850 | 3800309 | NM_022170.1 | NM_022170 | eukaryotic translation | EIF4H | KIAA0038; | GCACCCAGC | HSK |
| | | | | | | initiation factor 4H | | WSCR1; | GGAATGTGCT | |
| | | | | | | (EIF4H), transcript | | WBSCR1 | TAGTATTTGG | |
| | | | | | | variant 1, mRNA. | | | TCACCAGCC | |
| | | | | | | | | | GTCATCCTGG | |
| | | | | | | | | | GC | |
| 1721 | — | — | 1110017 | NM_032195.1 | NM_032195 | SON DNA binding | SON | FLJ21099; | GTGTTTAACC | HSK |
| | | | | | | protein (SON), | | SON3; | TAATGCTCAG | |
| | | | | | | transcript variant b, | | KIAA1019; | CCTTGGTACT | |
| | | | | | | mRNA. | | BASS1; | CCATTCCCTT | |
| | | | | | | | | NREBP; | CTCCTTCCCC | |
| | | | | | | | | C21orf50; | | |
| | | | | | | | | DBP-5; | | |
| | | | | | | | | FLJ33914 | | |
| 1722 | — | — | 2680097 | NM_016061.1 | NM_016061 | yippee-like 5 | YPEL5 | CGI-127 | GTGACTTCTG | HSK |
| | | | | | | (Drosophila) (YPEL5), | | | AGTACAGTTA | |
| | | | | | | mRNA. | | | AGTTCCTCCT | |
| | | | | | | | | | ATTTGCCACT | |
| | | | | | | | | | GGGCTGTTGG | |
| 1723 | — | — | 2480364 | NM_013379.2 | NM_013379 | dipeptidyl-peptidase 7 | DPP7 | DPP2; | TCACTCAAGC | HSK |
| | | | | | | (DPP7), mRNA. | | DPPII; QPP | AGCTGGCGG | |
| | | | | | | | | | CAGAGGGAA | |
| | | | | | | | | | GGGGCTGAA | |
| | | | | | | | | | TAAACGCCTG | |
| | | | | | | | | | GAG | |
| 1724 | — | — | 6330044 | NM_004034.1 | NM_004034 | annexin A7 (ANXA7), | ANXA7 | ANX7; SNX | ACTGAAAGCT | HSK |
| | | | | | | transcript variant 2, | | | CTGCCTTCCG | |
| | | | | | | mRNA. | | | GAATCCCTCT | |
| | | | | | | | | | AAGTCTGCTT | |
| | | | | | | | | | GATAGAGTGG | |
| 1725 | — | — | 240725 | NM_001033112.1 | NM_001033112 | poly(A) binding protein | PAIP2 | PAIP2A; | CTGAGGCTA | HSK |
| | | | | | | interacting protein 2 | | MGC72018 | CAAGTTAGTC | |
| | | | | | | (PAIP2), transcript | | | AGCAGATGA | |
| | | | | | | variant 1, mRNA. | | | GTGCCAGTC | |
| | | | | | | | | | CAGCCTTTTC | |
| | | | | | | | | | TGG | |
| 1726 | — | — | 3390192 | NM_006861.4 | NM_006861 | RAB35, member RAS | RAB35 | RAB1C; H- | GTGGGGACT | HSK |
| | | | | | | oncogene family | | ray; RAY | CAGGGCTGG | |
| | | | | | | (RAB35), mRNA. | | | ACCGACGTC | |
| | | | | | | | | | CTAGTGGAC | |
| | | | | | | | | | CTGATGTGAA | |
| | | | | | | | | | ATTC | |
| 1727 | — | — | 4150670 | NM_007065.3 | NM_007065 | cell division cycle 37 | CDC37 | P50CDC37 | CCGGCTCTC | HSK |
| | | | | | | homolog (S. cerevisiae) | | | GTCACTGGG | |
| | | | | | | (CDC37), | | | CTCTGTTTTC | |
| | | | | | | mRNA. | | | ACTGTTCGTC | |
| | | | | | | | | | TGCTGTCTGT | |
| | | | | | | | | | GT | |
| 1728 | — | — | 7200037 | NM_005626.3 | NM_005626 | splicing factor, | SFRS4 | SRP75 | TGGCCTTTCC | HSK |
| | | | | | | arginine/serine-rich 4 | | | TACAGGGAG | |
| | | | | | | (SFRS4), mRNA. | | | CTCAGTAACC | |
| | | | | | | | | | TGGACGGCT | |
| | | | | | | | | | CTAAGGCTG | |
| | | | | | | | | | GAA | |
| 1729 | — | — | 5690202 | NM_018064.2 | NM_018064 | chromosome 6 open | C6ORF166 | FLJ10342; | CAAGCTTTCG | HSK |
| | | | | | | reading frame 166 | | dJ486L4.2 | TCAGTGGCAA | |
| | | | | | | (C6orf166), mRNA. | | | CCACTCTTAG | |
| | | | | | | | | | GCAGCAGCA | |
| | | | | | | | | | ACTGGTTTTGG | |
| 1730 | — | — | 3830538 | NM_030818.2 | NM_030818 | coiled-coil domain | CCDC130 | MGC10471 | GGTCCTGGT | HSK |
| | | | | | | containing 130 | | | GAGGGTGTTT | |
| | | | | | | (CCDC130), mRNA. | | | GTGCCTTGTG | |
| | | | | | | | | | AGACTCCGTA | |
| | | | | | | | | | CATTAAAGACC | |
| 1731 | — | — | 2490066 | NM_006110.1 | NM_006110 | CD2 (cytoplasmic tail) | CD2BP2 | FWP010; | GCCCAGTTTG | HSK |
| | | | | | | binding protein 2 | | LIN1; | GTGGGCCCT | |
| | | | | | | (CD2BP2), mRNA. | | Snu40 | TCTTTCCTGG | |
| | | | | | | | | | ACTTTGTGGA | |
| | | | | | | | | | GGAGGCACC | |
| | | | | | | | | | AA | |
| 1732 | — | — | 4230050 | NM_006327.2 | NM_006327 | translocase of inner | TIMM23 | PRO1197; | TCCTCCCCCA | HSK |
| | | | | | | mitochondrial | | TIMM23B; | TGAACTAGAA | |
| | | | | | | membrane 23 homolog | | MGC22767; | AACCACTTAC | |
| | | | | | | (yeast) (TIMM23), | | TIM23 | TCCCAGAATT | |
| | | | | | | nuclear gene encoding | | | CAGGTCGTGC | |
| | | | | | | mitochondrial protein, | | | | |
| | | | | | | mRNA. | | | | |
| 1733 | — | — | 7200598 | NM_005466.2 | NM_005466 | mediator complex | MED6 | NY-REN-28 | CTTCTGTAAC | HSK |
| | | | | | | subunit 6 (MED6), | | | CTTTCCTCTC | |
| | | | | | | mRNA. | | | CCGGACTTG | |
| | | | | | | | | | AGCAACCTAC | |
| | | | | | | | | | ACACTCACATG | |
| 1734 | — | — | 6110477 | NM_006600.2 | NM_006600 | nuclear distribution | NUDC | NPD011; | CATCAGGAG | HSK |
| | | | | | | gene C homolog (A. nidulans) | | HNUDC; | AAAGGCTGG | |
| | | | | | | (NUDC), | | MNUDC | GTCTTGGGA | |
| | | | | | | mRNA. | | | CCTTGTCCTC | |
| | | | | | | | | | CCCAGTTGG | |
| | | | | | | | | | CCTA | |
| 1735 | — | — | 3130241 | NM_020141.3 | NM_020141 | chromosome 1 open | C1ORF119 | AD-020; | ACCAGTTTTT | HSK |
| | | | | | | reading frame 119 | | FLJ90710 | ACAGCCTCCT | |
| | | | | | | (C1orf119), mRNA. | | | GGGTGGGTC | |
| | | | | | | | | | GTCTTGACCC | |
| | | | | | | | | | AAACTCTTGTG | |
| 1736 | — | — | 60390 | NM_030914.1 | NM_030914 | ubiquitin related | URM1 | C9orf74; | CTGGGGAGA | HSK |
| | | | | | | modifier 1 homolog (S. cerevisiae) | | RP11- | TACTTGATGG | |
| | | | | | | (URM1), | | 339B21.4; | CGCGAATGT | |
| | | | | | | mRNA. | | MGC2668 | CCGTTTTCTC | |
| | | | | | | | | | TCCCTTCCCA | |
| | | | | | | | | | CC | |
| 1737 | — | — | 1450537 | NM_014607.3 | NM_014607 | UBX domain | UBXD2 | erasin; | GGCTGTAAAA | HSK |
| | | | | | | containing 2 (UBXD2), | | UBXDC1; | TGAGAATTCT | |
| | | | | | | mRNA. | | FLJ23318; | GCCCCCTCA | |
| | | | | | | | | KIAA0242 | CCTCTTACCC | |
| | | | | | | | | | CAGTACTATTC | |
| 1738 | — | — | 610112 | NM_173607.3 | NM_173607 | chromosome 14 open | C14ORF24 | DKFZp686J1254; | CTCCAGCCT | HSK |
| | | | | | | reading frame 24 | | FLJ38854 | GGGCGACAG | |
| | | | | | | (C14orf24), transcript | | | AGTGAGACTC | |
| | | | | | | variant 1, mRNA. | | | CATCTTGGG | |
| | | | | | | | | | GGGAAAAAA | |
| | | | | | | | | | GTAT | |

| RNA192 | | | | | |
|---|---|---|---|---|---|
| Index | | | | | |
| (SEQ | | | | | |
| ID NO: | | | | | |
| 1547- | Performance | | ABI Gene | | |
| 1738) | Rank | ABI Assay ID | Symbol | ABI Alias | ABI Gene Name |
| 1547 | 001N | Hs00229548_m1 | TMEM49 | DKFZp566I133, | transmembrane |
| | | | | HSPC292, VMP1 | protein 49 |
| 1548 | 001P | Hs00360269_m1 | VAMP2 | FLJ11460, | vesicle- |
| | | | | SYB2, VAMP-2 | associated |
| | | | | | membrane |
| | | | | | protein 2 |
| | | | | | (synaptobrevin 2) |
| 1549 | 002N | Hs00240532_s1 | ADRB2 | ADRB2R, | adrenergic, |
| | | | | ADRBR, B2AR, | beta-2-, |
| | | | | BAR, | receptor, |
| | | | | BETA2AR | surface |
| 1550 | 002P | Hs00270173_s1 | NAP1L3 | MB20, MGC26312, NPL3, | nucleosome |
| | | | | RP1- | assembly |
| | | | | 32F7.3 | protein 1-like 3 |
| 1551 | 003N | Hs00967385_g1 | SERBP1 | CGI- | SERPINE1 |
| | | | | 55, CHD3IP, | mRNA |
| | | | | DKFZp564M2423, | binding |
| | | | | FLJ90489, | protein |
| | | | | HABP4L, | |
| | | | | PAI- | |
| | | | | RBP1, PAIRBP1 | |
| 1552 | 003P | Hs00211126_m1 | CSAD | CSD, FLJ44987, | cysteine |
| | | | | FLJ45500, MGC119354, | sulfinic acid |
| | | | | MGC119355, | decarboxylase |
| | | | | MGC119357, | |
| | | | | PCAP | |
| 1553 | 004N | Hs00247361_m1 | NPTN | DKFZp686L2477, GP55, | neuroplastin |
| | | | | GP65, | |
| | | | | MGC102805, | |
| | | | | SDFR1, | |
| | | | | SDR1, | |
| | | | | np55, np65 | |
| 1554 | 004P | Hs00937468_m1 | AEBP1 | ACLP, FLJ33612 | AE binding |
| | | | | | protein 1 |
| 1555 | 005N | Hs00233566_m1 | CD79A | IGA, MB-1 | CD79a |
| | | | | | molecule, |
| | | | | | immunoglobulin- |
| | | | | | associated |
| | | | | | alpha |
| 1556 | 005P | Hs02340038_g1 | RPL37 | DKFZp686G1699, | ribosomal |
| | | | | MGC99572 | protein L37 |
| 1557 | 006N | Hs00175132_m1 | ANXA11 | ANX11, CAP50, | annexin A11 |
| | | | | RP11- | |
| | | | | 369J21.10- | |
| | | | | 010 | |
| 1558 | 006P | Hs00959834_m1 | XRCC1 | RCC | X-ray repair |
| | | | | | complementing |
| | | | | | defective |
| | | | | | repair in |
| | | | | | Chinese |
| | | | | | hamster cells 1 |
| 1559 | 007N | Hs00227769_m1 | POF1B | FLJ22792, | premature |
| | | | | POF, POF2B, | ovarian |
| | | | | RP1- | failure, 1B |
| | | | | 75N13.2 | |
| 1560 | 007P | Hs00255244_m1 | RABL2B | FLJ93981, | RAB, member |
| | | | | FLJ98216, | of RAS |
| | | | | FLJ78724, | oncogene |
| | | | | MGC117180, | family-like |
| | | | | RP11- | 2B, RAB, |
| | | | | 395L14.2 | member of |
| | | | | | RAS |
| | | | | | oncogene |
| | | | | | family-like 2A |
| 1561 | 008N | Hs00229472_m1 | SNX27 | KIAA0488, | sorting nexin |
| | | | | MGC126871, | family |
| | | | | MGC126873, MGC20471, | member 27 |
| | | | | MRT1, MY014, | |
| | | | | RP11- | |
| | | | | 98D18.12- | |
| | | | | 005 | |
| 1562 | 008P | Hs00369741_m1 | SELM | MGC40146; | selenoprotein M |
| | | | | SEPM | |
| 1563 | 009N | Hs01033772_g1 | NCOA4 | ARA70, DKFZp762E1112, | nuclear |
| | | | | ELE1, | receptor |
| | | | | PTC3, | coactivator 4 |
| | | | | RFG, RP11- | |
| | | | | 481A12.4 | |
| 1564 | 009P | Hs01127828_g1 | HMGB2 | HMG2 | high-mobility |
| | | | | | group box 2 |
| 1565 | 010N | Hs00418081_m1 | C22orf33 | EAN57, LL22NC01- | chromosome |
| | | | | 81G9.2, MGC35206, | 22 open |
| | | | | cE81G9.2 | reading frame |
| | | | | | 33 |
| 1566 | 010P | Hs00380060_m1 | TMC8 | EV2, EVER2, | transmembrane |
| | | | | EVIN2, | channel- |
| | | | | FLJ40668, | like 8 |
| | | | | FLJ43684, | |
| | | | | MGC102701, MGC40121 | |
| 1567 | 011N | Hs00292978_m1 | KIAA1949 | DAAP- | KIAA1949 |
| | | | | 285E11.2, | |
| | | | | HKMT1098 | |
| 1568 | 011P | Hs00270322_m1 | TAF1 | BA2R, CCG1, | TAF1 RNA |
| | | | | CCGS, | polymerase II, |
| | | | | DYT3, DYT3/ | TATA box |
| | | | | TAF1, | binding |
| | | | | KAT4, N- | protein (TBP)- |
| | | | | TAF1, NSCL2, | associated |
| | | | | OF, P250, | factor, |
| | | | | TAF2A, | 250 kDa |
| | | | | TAFII250, | |
| | | | | XDP | |
| 1569 | 012N | Hs00218034_m1 | ACER3 | APHC, FLJ11238, PHCA | alkaline |
| | | | | | ceramidase 3 |
| 1570 | 012P | Hs00176278_m1 | DGKA | DAGK, DAGK1, | diacylglycerol |
| | | | | DGK- | kinase, alpha |
| | | | | alpha, MGC12821, MGC42356 | 80 kDa |
| 1571 | 013N | Hs00377534_m1 | TTC21A | DKFZp686P18239, | tetratricopeptide |
| | | | | MGC156293, | repeat |
| | | | | MGC70523, | domain 21A |
| | | | | STI2 | |
| 1572 | 013P | Hs00251360_s1 | STRADB | ALS2CR2, | STE20- |
| | | | | CALS- | related kinase |
| | | | | 21, ILPIP, ILPIPA, MGC102916, | adaptor beta |
| | | | | PAPK, PRO1038 | |
| 1573 | 014N | Hs02825719_s1 | GPR119 | GPCR2, MGC119957, | G protein- |
| | | | | RP1- | coupled |
| | | | | 20I3.4 | receptor 119 |
| 1574 | 014P | Hs00427396_m1 | PSMD7 | MOV34, P40, | proteasome |
| | | | | Rpn8, | (prosome, |
| | | | | S12 | macropain) |
| | | | | | 26S subunit, |
| | | | | | non-ATPase, 7 |
| 1575 | 015N | Hs00221046_m1 | SENP7 | KIAA1707, | SUMO1/sentrin |
| | | | | MGC157730 | specific |
| | | | | | peptidase 7 |
| 1576 | 015P | Hs00357776_g1 | GRK6 | FLJ32135, | G protein- |
| | | | | GPRK6 | coupled |
| | | | | | receptor |
| | | | | | kinase 6 |
| 1577 | 016N | Hs01018047_m1 | ABHD12 | ABHD12A, | abhydrolase |
| | | | | BEM46L2, | domain |
| | | | | C20ORF22, | containing 12 |
| | | | | DKFZp434P106, | |
| | | | | RP5- | |
| | | | | 965G21.2, | |
| | | | | dJ965G21.2 | |
| 1578 | 016P | Hs00757030_m1 | PRPF3 | HPRP3, HPRP3P, PRP3, | PRP3 pre- |
| | | | | Prp3p, | mRNA |
| | | | | RP18 | processing |
| | | | | | factor 3 |
| | | | | | homolog (S. cerevisiae) |
| 1579 | 017N | Hs01850784_s1 | FOXG1 | BF1, BF2, | forkhead box |
| | | | | FHKL3, FKH2, | G1 |
| | | | | FKHL1, | |
| | | | | FKHL2, | |
| | | | | FKHL3, FKHL4, | |
| | | | | FOXG1A, | |
| | | | | FOXG1B, | |
| | | | | FOXG1C, | |
| | | | | HBF- | |
| | | | | 1, HBF- | |
| | | | | 2, HBF- | |
| | | | | 3, HBF- | |
| | | | | G2, HBF2, | |
| | | | | HFK1, HFK2, | |
| | | | | HFK3, | |
| | | | | KHL2, QIN | |
| 1580 | 017P | Hs00538167_m1 | IL12RB1 | CD212, IL- | interleukin 12 |
| | | | | 12R- | receptor, beta 1 |
| | | | | BETA1, IL12RB, | |
| | | | | MGC34454 | |
| 1581 | 018N | Hs02339636_g1 | TIMM8B | DDP2, FLJ21744, MGC102866, | translocase of |
| | | | | MGC117373, | inner |
| | | | | TIM8B | mitochondrial |
| | | | | | membrane 8 |
| | | | | | homolog B |
| | | | | | (yeast) |
| 1582 | 018P | Hs00262564_m1 | ZFYVE19 | FLJ14840, | zinc finger, |
| | | | | MPFYVE | FYVE domain |
| | | | | | containing 19 |
| 1583 | 019N | Hs01035151_m1 | CRTAP | CASP, LEPREL3 | cartilage |
| | | | | | associated |
| | | | | | protein |
| 1584 | 019P | Hs00364437_m1 | SCAND2 | 0 | SCAN domain |
| | | | | | containing 2 |
| | | | | | pseudogene |
| 1585 | 020N | Hs00198882_m1 | LAPTM5 | CLAST6, | lysosomal |
| | | | | FLJ61683, | protein |
| | | | | FLJ97251, | transmembrane 5 |
| | | | | MGC125860, | |
| | | | | MGC125861, | |
| | | | | RP5- | |
| | | | | 1166H10.3 | |
| 1586 | 020P | Hs00930964_g1 | PLAC8 | BM- | placenta- |
| | | | | 004, C15, onzin | specific 8 |
| 1587 | 021N | Hs00243566_m1 | B4GALT2 | B4Gal- | UDP- |
| | | | | T2, B4Gal- | Gal:betaGlcNAc |
| | | | | T3, beta4 | beta 1,4- |
| | | | | Gal-T2 | galactosyltransferase, |
| | | | | | polypeptide 2 |
| 1588 | 021P | Hs00195582_m1 | S100A2 | CAN19, MGC111539, | S100 calcium |
| | | | | RP11- | binding |
| | | | | 49N14.8, | protein A2 |
| | | | | S100L | |
| 1589 | 022N | Hs00227643_m1 | ERMP1 | FXNA, KIAA1815, | endoplasmic |
| | | | | RP11- | reticulum |
| | | | | 207C16.6, | metallopeptidase 1 |
| | | | | bA207C16.3 | |
| 1590 | 022P | Hs00159587_m1 | NDUFC1 | KFYI, MGC117464, | NADH |
| | | | | MGC126847, | dehydrogenase |
| | | | | MGC138266 | (ubiquinone) |
| | | | | | 1, |
| | | | | | subcomplex |
| | | | | | unknown, 1, |
| | | | | | 6 kDa |
| 1591 | 023N | Hs00609198_m1 | GCH1 | DYT14, DYT5, | GTP |
| | | | | DYT5a, | cyclohydrolase 1 |
| | | | | GCH, | |
| | | | | GTP-CH- | |
| | | | | 1, GTPCH1, | |
| | | | | HPABH4B | |
| 1592 | 023P | Hs00197131_m1 | PIBF1 | C13ORF24, | progesterone |
| | | | | KIAA1008, | immunomodulatory |
| | | | | PIBF, RP11- | binding |
| | | | | 505F3.1 | factor 1 |
| 1593 | 024N | Hs00377132_m1 | ZNF296 | ZFP296, ZNF342 | zinc finger |
| | | | | | protein 296 |
| 1594 | 024P | Hs00255552_m1 | TMEM8B | C9ORF127, | transmembrane |
| | | | | MGC120460, | protein 8B |
| | | | | NAG- | |
| | | | | 5, NGX6, RP11- | |
| | | | | 112J3.10, | |
| | | | | RP11- | |
| | | | | 112J3.10- | |
| | | | | 001 | |
| 1595 | 025N | Hs00300724_m1 | EPHA5 | CEK7, EHK1, | EPH receptor |
| | | | | HEK7, | A5 |
| | | | | TYRO4 | |
| 1596 | 025P | Hs01127577_m1 | CBX5 | HP1, HP1A | chromobox |
| | | | | | homolog 5 |
| 1597 | 026N | Hs00367123_m1 | AP2A1 | ADTAA, AP2- | adaptor- |
| | | | | ALPHA, CLAPA1 | related protein |
| | | | | | complex 2, |
| | | | | | alpha 1 |
| | | | | | subunit |
| 1598 | 026P | Hs00179978_m1 | EPS15 | AF- | epidermal |
| | | | | 1P, AF1P, | growth factor |
| | | | | MLLT5 | receptor |
| | | | | | pathway |
| | | | | | substrate 15 |
| 1599 | 027N | Hs00195343_m1 | SMNDC1 | SMNR, SPF30 | survival motor |
| | | | | | neuron |
| | | | | | domain |
| | | | | | containing 1 |
| 1600 | 027P | Hs00223885_m1 | MPP5 | FLJ12615, | membrane |
| | | | | PALS1 | protein, |
| | | | | | palmitoylated |
| | | | | | 5 (MAGUK |
| | | | | | p55 subfamily |
| | | | | | member 5) |
| 1601 | 028N | Hs00377608_m1 | NFAM1 | CNAIP, CTA- | NFAT |
| | | | | 126B4.4, FLJ40652, | activating |
| | | | | bK126B4.4 | protein with |
| | | | | | ITAM motif 1 |
| 1602 | 028P | Hs00275076_m1 | HEMK1 | FLJ22320, | HemKmethyltransferase |
| | | | | HEMK, MTQ1 | family |
| | | | | | member 1 |
| 1603 | 029N | Hs00893626_m1 | IL1RN | DIRA, ICIL- | interleukin 1 |
| | | | | 1RA, IL- | receptor |
| | | | | 1RN, IL- | antagonist |
| | | | | 1ra, IL- | |
| | | | | 1ra3, IL1F3, | |
| | | | | IL1RA, IRAP, | |
| | | | | MGC10430, MVCD4 | |
| 1604 | 029P | Hs00902008_m1 | CTCF | 0 | CCCTC- |
| | | | | | binding factor |
| | | | | | (zinc finger |
| | | | | | protein) |
| 1605 | 030N | Hs00164932_m1 | ICAM1 | BB2, CD54, | intercellular |
| | | | | P3.58 | adhesion |
| | | | | | molecule 1 |
| 1606 | 030P | Hs00293954_m1 | C10orf35 | RP11- | chromosome |
| | | | | 343J3.9 | 10 open |
| | | | | | reading frame |
| | | | | | 35 |
| 1607 | 031N | Hs00193519_m1 | MAF | MGC71685, | v- |
| | | | | c-MAF | mafmusculoaponeuroticfibrosarcoma |
| | | | | | oncogene |
| | | | | | homolog |
| | | | | | (avian) |
| 1608 | 031P | Hs00181740_m1 | FLT3LG | FL | fms-related |
| | | | | | tyrosine |
| | | | | | kinase 3 |
| | | | | | ligand |
| 1609 | 032N | Hs00248380_m1 | MAST2 | FLJ39200, | microtubule |
| | | | | KIAA0807, | associated |
| | | | | MAST205, | serine/threonine |
| | | | | MTSSK, | kinase 2 |
| | | | | RP4- | |
| | | | | 533D7.1 | |
| 1610 | 032P | Hs00209335_m1 | MYCBP2 | AC001226.5, | MYC binding |
| | | | | DKFZp686M08244, | protein 2 |
| | | | | FLJ10106, | |
| | | | | FLJ13826, | |
| | | | | FLJ21597, FLJ21646, | |
| | | | | KIAA0916, | |
| | | | | PAM | |
| 1611 | 033N | Hs00376245_m1 | GSG1L | MGC18079, | GSG1-like |
| | | | | PRO19651, | |
| | | | | UNQ5831 | |
| 1612 | 033P | Hs00212889_m1 | PPHLN1 | HSPC206, | periphilin 1 |
| | | | | HSPC232, | |
| | | | | MGC48786 | |
| 1613 | 034N | Hs00186575_m1 | PPAP2C | LPP2, PAP- | phosphatidic |
| | | | | 2c, PAP2-g | acid |
| | | | | | phosphatase |
| | | | | | type 2C |
| 1614 | 034P | Hs01122781_m1 | KIF3B | HH0048, KIAA0359 | kinesin family |
| | | | | | member 3B |
| 1615 | 035N | Hs00364814_m1 | IL22RA2 | CRF2- | interleukin 22 |
| | | | | 10, CRF2- | receptor, |
| | | | | S1, CRF2X, | alpha 2 |
| | | | | IL- | |
| | | | | 22BP, IL- | |
| | | | | 22RA2, MGC150509, | |
| | | | | MGC150510, | |
| | | | | UNQ5793/PRO19598/ | |
| | | | | PRO19822 | |
| 1616 | 035P | Hs00912503_m1 | PIGO | DKFZp434M222, | phosphatidylinositol |
| | | | | FLJ00135, MGC20536, | glycan |
| | | | | MGC3079, | anchor |
| | | | | RP11- | biosynthesis, |
| | | | | 182N22.4, | class O |
| | | | | UNQ632/ | |
| | | | | PRO1249 | |
| 1617 | 036N | Hs00244603_m1 | SERPINB9 | CAP- | serpin |
| | | | | 3, CAP3, PI9 | peptidase |
| | | | | | inhibitor, clade |
| | | | | | B (ovalbumin), |
| | | | | | member 9 |
| 1618 | 036P | Hs01053640_m1 | TXK | BTKL, MGC22473, PSCTK5, PTK4, | TXK tyrosine |
| | | | | RLK, | kinase |
| | | | | TKL | |
| 1619 | 037N | Hs00368207_m1 | PREX1 | KIAA1415, | phosphatidylinositol- |
| | | | | P-REX1 | 3,4,5- |
| | | | | | trisphosphate- |
| | | | | | dependent |
| | | | | | Rac exchange |
| | | | | | factor 1 |
| 1620 | 037P | Hs00268260_m1 | SMARCB1 | BAF47, INI1, | SWI/SNF |
| | | | | RDT, RTPS1, | related, matrix |
| | | | | SNF5, | associated, |
| | | | | SNF5L1, | actin |
| | | | | Sfh1p, Snr1, | dependent |
| | | | | hSNFS | regulator of |
| | | | | | chromatin, |
| | | | | | subfamily b, |
| | | | | | member 1 |
| 1621 | 038N | Hs00216128_m1 | NSUN5 | FLJ10267, | NOP2/Sun |
| | | | | MGC986, | domain family, |
| | | | | NOL1, NOL1R, | member 5 |
| | | | | NSUN5A, | |
| | | | | WBSCR20, | |
| | | | | WBSCR20A, | |
| | | | | p120 | |
| 1622 | 038P | Hs00204112_m1 | MRPL42 | HSPC204, | mitochondrial |
| | | | | MRP- | ribosomal |
| | | | | L31, MRPL31, | protein L42 |
| | | | | MRPS32, | |
| | | | | PTD007, | |
| | | | | RPML31 | |
| 1623 | 039N | Hs00384853_m1 | SIPA1L2 | FLJ23126, | signal-induced |
| | | | | FLJ23632, | proliferation- |
| | | | | KIAA1389, | associated 1 |
| | | | | SPAL2 | like 2 |
| 1624 | 039P | Hs00234934_m1 | CACNA1H | CACNA1HB, | calcium |
| | | | | Cav3.2, | channel, |
| | | | | ECA6, EIG6, | voltage- |
| | | | | FLJ90484 | dependent, T |
| | | | | | type, alpha 1H |
| | | | | | subunit |
| 1625 | 040N | Hs00829622_s1 | ZFAND5 | RP11- | zinc finger, |
| | | | | 63P12.8, ZA20D2, | AN1-type |
| | | | | ZFAND5A, ZNF216 | domain 5 |
| 1626 | 040P | Hs00183813_m1 | PDCD6IP | AIP1, Alix, | programmed |
| | | | | DRIP4, HP95, | cell death 6 |
| | | | | MGC17003 | interacting |
| | | | | | protein |
| 1627 | 041N | Hs00379444_m1 | NCDN | KIAA0607 | neurochondrin |
| 1628 | 041P | Hs00762282_s1 | VTI1B | VTI1, VTI1- | vesicle |
| | | | | LIKE, VTI1L, | transport |
| | | | | VTI2 | through |
| | | | | | interaction |
| | | | | | with t- |
| | | | | | SNAREs |
| | | | | | homolog 1B |
| | | | | | (yeast) |
| 1629 | 042N | Hs00923916_m1 | SALL3 | ZNF796 | sal-like 3 |
| | | | | | (Drosophila) |
| 1630 | 042P | Hs00757279_mH | TATDN1 | CDA11, FLJ43280 | TatDDNase |
| | | | | | domain |
| | | | | | containing 1 |
| 1631 | 043N | Hs00193731_m1 | CCDC6 | D10S170, | coiled-coil |
| | | | | FLJ32286, | domain |
| | | | | H4, PTC, | containing 6 |
| | | | | TPC, TST1 | |
| 1632 | 043P | Hs00260456_m1 | COQ5 | MGC104303, | coenzyme Q5 |
| | | | | MGC4767 | homolog, |
| | | | | | methyltransferase |
| | | | | | (S. cerevisiae) |
| 1633 | 044N | Hs00939664_m1 | FOXN2 | HTLF | forkhead box |
| | | | | | N2 |
| 1634 | 044P | Hs01126016_m1 | UTRN | DMDL, DRP, | utrophin |
| | | | | DRP1, FLJ23678, | |
| | | | | RP11- | |
| | | | | 352E13.1 | |
| 1635 | 045N | Hs01115513_m1 | PPARG | CIMT1, GLM1, | peroxisome |
| | | | | NR1C3, | proliferator- |
| | | | | PPARG1, | activated |
| | | | | PPARG2, | receptor |
| | | | | PPARgamma | gamma |
| 1636 | 045P | Hs00219487_m1 | CCDC76 | FLJ10287, | coiled-coil |
| | | | | FLJ11219, | domain |
| | | | | RP11- | containing 76 |
| | | | | 305E17.1 | |
| 1637 | 046N | Hs00212858_m1 | ANAPC11 | APC11, Apc11p, | anaphase |
| | | | | HSPC214, MGC882 | promoting |
| | | | | | complex |
| | | | | | subunit 11 |
| 1638 | 046P | Hs00903035_g1 | EI24 | PIG8, TP53I8 | etoposide |
| | | | | | induced 2.4 |
| | | | | | mRNA |
| 1639 | 047N | Hs00427977_m1 | ERH | DROER, FLJ27340 | enhancer of |
| | | | | | rudimentary |
| | | | | | homolog |
| | | | | | (Drosophila) |
| 1640 | 047P | Hs00383486_m1 | CC2D1B | KIAA1836, | coiled-coil and |
| | | | | RP11- | C2 domain |
| | | | | 155O18.2 | containing 1B |
| 1641 | 048N | Hs00295839_m1 | RCHY1 | ARNIP, CHIMP, | ring finger and |
| | | | | DKFZp586C1620, | CHY zinc |
| | | | | PIRH2, | finger domain |
| | | | | PRO1996, | containing 1 |
| | | | | RNF199, | |
| | | | | ZNF363, | |
| | | | | hARNIP | |
| 1642 | 048P | Hs00197392_m1 | TM9SF1 | HMP70, MP70 | transmembrane 9 |
| | | | | | superfamily |
| | | | | | member 1 |
| 1643 | 049N | Hs01651150_m1 | ENTPD8 | GLSR2492, | ectonucleoside |
| | | | | NTPDase- | triphosphate |
| | | | | 8, UNQ2492, | diphosphohydrolase 8 |
| | | | | UNQ2492/ | |
| | | | | PRO5779 | |
| 1644 | 049P | Hs00398565_m1 | C3orf15 | AAT1, AAT1alpha, DKFZp781A2221 | chromosome |
| | | | | | 3 open |
| | | | | | reading frame |
| | | | | | 15 |
| 1645 | 050N | Hs00381867_m1 | FGFR1OP2 | DKFZp564O1863, DKFZp586C1423, | FGFR1 |
| | | | | FLJ37569, HSPC123, | oncogene |
| | | | | HSPC123- | partner 2 |
| | | | | like, WIT3.0 | |
| 1646 | 050P | Hs00274505_m1 | NCAPD2 | CAP- | non-SMC |
| | | | | D2, CNAP1, | condensin I |
| | | | | KIAA0159, | complex, |
| | | | | hCAP- | subunit D2 |
| | | | | D2 | |
| 1647 | 051N | Hs00152825_m1 | TLR5 | FLJ10052, | toll-like |
| | | | | MGC126430, | receptor 5 |
| | | | | MGC126431, RP11- | |
| | | | | 239E10.1, | |
| | | | | SLEB1, TIL3 | |
| 1648 | 051P | Hs00212852_m1 | C20orf111 | BM- | chromosome |
| | | | | 038, HSPC207, | 20 open |
| | | | | Perit1, | reading frame |
| | | | | dJ1183I21.1 | 111 |
| 1649 | 052N | Hs00542678_m1 | NFATC1 | MGC138448, | nuclear factor |
| | | | | NF- | of activated T- |
| | | | | ATC, NFAT2, | cells, |
| | | | | NFATc | cytoplasmic, |
| | | | | | calcineurin- |
| | | | | | dependent 1 |
| 1650 | 052P | Hs00226305_m1 | ARHGAP10 | FLJ20896, | Rho GTPase |
| | | | | FLJ41791, | activating |
| | | | | GRAF2, | protein 10 |
| | | | | PS- | |
| | | | | GAP, PSGAP | |
| 1651 | 053N | Hs00369703_m1 | RPUSD1 | C16ORF40, | RNA |
| | | | | MGC19600, | pseudouridylate |
| | | | | RLUCL | synthase |
| | | | | | domain |
| | | | | | containing 1 |
| 1652 | 053P | Hs00418963_m1 | XPO1 | CRM1, DKFZp686B1823, | exportin 1 |
| | | | | emb | (CRM1 |
| | | | | | homolog, |
| | | | | | yeast) |
| 1653 | 054N | Hs00226971_m1 | MMRN2 | EMILIN3, | multimerin 2 |
| | | | | EndoGlyx- | |
| | | | | 1, FLJ13465 | |
| 1654 | 054P | Hs00212785_m1 | MPP6 | PALS2, VAM- | membrane |
| | | | | 1, VAM1, p55T | protein, |
| | | | | | palmitoylated |
| | | | | | 6 (MAGUK |
| | | | | | p55 subfamily |
| | | | | | member 6) |
| 1655 | 055N | Hs00185020_m1 | AQP3 | GIL | aquaporin 3 |
| | | | | | (Gill blood |
| | | | | | group) |
| 1656 | 055P | Hs00197140_m1 | COG5 | CDG2I, FLJ41732, FLJ44289, | component of |
| | | | | GOLTC1, | oligomericgolgi |
| | | | | GTC90 | complex 5 |
| 1657 | 056N | Hs00368084_m1 | RARS2 | ArgRS, DALRD2, | arginyl- |
| | | | | MGC14993, MGC23778, | tRNAsynthetase |
| | | | | PCH6, PRO1992, RARSL, | 2, |
| | | | | RP3- | mitochondrial |
| | | | | 382l10.6, | |
| | | | | dJ382l10.6 | |
| 1658 | 056P | Hs01582977_gH | MT1E | MT1, MTD | metallothionein |
| | | | | | 1E |
| 1659 | 057N | Hs00217534_m1 | WDR41 | FLJ10904, | WD repeat |
| | | | | MSTP048 | domain 41 |
| 1660 | 057P | Hs01597912_g1 | RANBP1 | HTF9A, MGC88701 | RAN binding |
| | | | | | protein 1 |
| 1661 | 058N | Hs00872692_m1 | NRK | DKFZp686A17109, | Nik related |
| | | | | FLJ16788, | kinase |
| | | | | MGC131849, | |
| | | | | NESK, | |
| | | | | RP1- | |
| | | | | 82J11.1 | |
| 1662 | 058P | Hs00275795_m1 | SPG7 | CAR, CMAR, | spastic |
| | | | | FLJ37308, | paraplegia 7 |
| | | | | MGC126331, MGC126332, | (pure and |
| | | | | PGN, SPG5C | complicated |
| | | | | | autosomal |
| | | | | | recessive) |
| 1663 | 059N | Hs00198472_m1 | SRCAP | DOMO1, | Snf2-related |
| | | | | EAF1, FLJ44499, | CREBBP |
| | | | | KIAA0309, SWR1 | activator |
| | | | | | protein |
| 1664 | 059P | Hs00204546_m1 | TMEM5 | HP10481 | transmembrane |
| | | | | | protein 5 |
| 1665 | 060N | Hs00165902_m1 | TCN2 | D22S676, | transcobalamin |
| | | | | D22S750, | II |
| | | | | II, TC, TCII, | |
| | | | | TC- | |
| | | | | 2, TC2, TCII | |
| 1666 | 060P | Hs00248408_m1 | SEPT6 | KIAA0128, | septin 6 |
| | | | | MGC16619, | |
| | | | | MGC20339, | |
| | | | | RP5- | |
| | | | | 876A24.2, | |
| | | | | SEP2, SEPT2 | |
| 1667 | 061N | Hs00203383_m1 | C7orf68 | FLJ21076, | chromosome |
| | | | | HIG- | 7 open |
| | | | | 2, HIG2, MGC138388 | reading frame |
| | | | | | 68 |
| 1668 | 061P | Hs00208618_m1 | AAK1 | DKFZp686F03202, | AP2 |
| | | | | DKFZp686K16132, | associated |
| | | | | FLJ23712, | kinase 1 |
| | | | | FLJ25931, | |
| | | | | FLJ31060, | |
| | | | | FLJ42882, | |
| | | | | FLJ45252, | |
| | | | | KIAA1048, | |
| | | | | MGC138170, | |
| | | | | MGC164568, | |
| | | | | MGC164570 | |
| 1669 | 062N | Hs00163869_m1 | CA2 | CA- | carbonic |
| | | | | II, CAII, Car2 | anhydrase II |
| 1670 | 062P | Hs00535769_m1 | C8orf33 | FLJ20989 | chromosome |
| | | | | | 8 open |
| | | | | | reading frame |
| | | | | | 33 |
| 1671 | 063N | Hs00610137_m1 | STAM | DKFZp686J2352, STAM1 | signal |
| | | | | | transducing |
| | | | | | adaptor |
| | | | | | molecule |
| | | | | | (SH3 domain |
| | | | | | and ITAM |
| | | | | | motif) 1 |
| 1672 | 063P | Hs00224208_m1 | SMYD3 | FLJ21080, | SET and |
| | | | | KMT3E, | MYND |
| | | | | MGC104324, | domain |
| | | | | ZMYND1, | containing 3 |
| | | | | ZNFN3A1, | |
| | | | | bA74P14.1 | |
| 1673 | 064N | Hs00608163_m1 | SEC14L1 | DKFZp686C06176, | SEC14-like 1 |
| | | | | PRELID4A, | (S. cerevisiae) |
| | | | | SEC14L | |
| 1674 | 064P | Hs00265266_g1 | GSTM2 | GST4, GSTM, | glutathione S- |
| | | | | GSTM2- | transferase |
| | | | | 2, GTHMUS, | mu 2 (muscle) |
| | | | | MGC117303 | |
| 1675 | 065N | Hs00698292_m1 | FADS6 | FP18279 | fatty acid |
| | | | | | desaturase |
| | | | | | domain family, |
| | | | | | member 6 |
| 1676 | 065P | Hs00853882_g1 | SNRPC | FLJ20302, | small nuclear |
| | | | | RP3- | ribonucleoprotein |
| | | | | 375P9.1, | polypeptide C |
| | | | | U1C, Yhc1 | |
| 1677 | 066N | Hs01040835_m1 | GINS1 | KIAA0186, | GINS |
| | | | | PSF1, RP4- | complex |
| | | | | 4- | subunit 1 |
| | | | | 691N24.2 | (Psf1 |
| | | | | | homolog) |
| 1678 | 066P | Hs00270914_m1 | CALM3 | CALM1, CALM2, | calmodulin 3 |
| | | | | PHKD, | (phosphorylase |
| | | | | PHKD3 | kinase, |
| | | | | | delta) |
| 1679 | 067N | Hs00363121_m1 | POLR3K | C11, C11- | polymerase |
| | | | | RNP3, My010, | (RNA) III |
| | | | | RPC10, | (DNA |
| | | | | RPC11, | directed) |
| | | | | RPC12.5, | polypeptide K, |
| | | | | hRPC11 | 12.3 kDa |
| 1680 | 067P | Hs00208576_m1 | RNF44 | KIAA1100 | ring finger |
| | | | | | protein 44 |
| 1681 | 068N | Hs00362067_m1 | COX5A | COX, COX-VA, | cytochrome c |
| | | | | VA | oxidase |
| | | | | | subunit Va |
| 1682 | 068P | Hs00536084_m1 | PHAX | FLJ13193, | phosphorylated |
| | | | | RNUXA | adaptor for |
| | | | | | RNA export |
| 1683 | 069N | Hs00220260_m1 | RNPEP | DKFZp547H084 | arginylaminopeptidase |
| | | | | | (aminopeptidase |
| | | | | | B) |
| 1684 | 069P | Hs00608563_m1 | HEATR3 | FLJ20718 | HEAT repeat |
| | | | | | containing 3 |
| 1685 | 070N | Hs00260900_m1 | C5orf32 | ORF1- | chromosome |
| | | | | FL49 | 5 open |
| | | | | | reading frame |
| | | | | | 32 |
| 1686 | 070P | Hs00156055_m1 | BCL7B | 0 | B-cell |
| | | | | | CLL/lymphoma |
| | | | | | 7B |
| 1687 | 071N | Hs00246261_m1 | HBXIP | MGC71071, | hepatitis B |
| | | | | XIP | virus x |
| | | | | | interacting |
| | | | | | protein |
| 1688 | 071P | Hs00217433_m1 | YY1AP1 | FLJ10875, | YY1 |
| | | | | FLJ13914, | associated |
| | | | | HCCA1, | protein 1 |
| | | | | HCCA2, RP11- | |
| | | | | 243J18.1, | |
| | | | | YAP, YY1AP | |
| 1689 | 072N | Hs00170832_m1 | CD226 | DNAM- | CD226 |
| | | | | 1, DNAM1, | molecule |
| | | | | PTA1, TLiSA1 | |
| 1690 | 072P | Hs01075391_m1 | ZNF641 | DKFZp667D1012, FLJ31295 | zinc finger |
| | | | | | protein 641 |
| 1691 | 073N | Hs00204415_m1 | HOXC11 | HOX3H, MGC4906 | homeobox |
| | | | | | C11 |
| 1692 | 073P | Hs00971557_m1 | KLF12 | AP- | Kruppel-like |
| | | | | 2rep, AP2REP, | factor 12 |
| | | | | HSPC122 | |
| 1693 | 074N | Hs00225908_m1 | C16orf53 | FLJ22459, | chromosome |
| | | | | GAS, MGC4606, | 16 open |
| | | | | PA1 | reading frame |
| | | | | | 53 |
| 1694 | 074P | Hs00248344_m1 | SARM1 | FLJ36296, | sterile alpha |
| | | | | KIAA0524, | and TIR motif |
| | | | | SAMD2, | containing 1 |
| | | | | SARM | |
| 1695 | 075N | Hs00905983_m1 | N4BP2 | B3BP, FLJ10680, | NEDD4 |
| | | | | KIAA1413 | binding |
| | | | | | protein 2 |
| 1696 | 075P | Hs01390827_g1 | LYRM5 | 0 | LYR motif |
| | | | | | containing 5 |
| 1697 | 076N | Hs00541038_m1 | SHMT1 | CSHMT, | serine |
| | | | | MGC15229, | hydroxymethyltransferase 1 |
| | | | | MGC24556, | (soluble) |
| | | | | SHMT | |
| 1698 | 076P | Hs00823168_g1 | MT1H | MGC70702, | metallothionein |
| | | | | MT1 | 1H |
| 1699 | 077N | Hs00172870_m1 | NR2F6 | EAR- | nuclear |
| | | | | 2, EAR2, ERBAL2 | receptor |
| | | | | | subfamily 2, |
| | | | | | group F, |
| | | | | | member 6 |
| 1700 | 077P | Hs02518187_g1 | PNRC2 | FLJ20312, | proline-rich |
| | | | | MGC99541, | nuclear |
| | | | | RP11- | receptor |
| | | | | 4M23.5 | coactivator 2 |
| 1701 | 078N | Hs00410739_m1 | DGKH | DGKeta, DKFZp761I1510, | diacylglycerol |
| | | | | RP11- | kinase, eta |
| | | | | 215B13.1 | |
| 1702 | 078P | Hs01122981_m1 | PJA2 | KIAA0438, | praja ring |
| | | | | Neurodap1, | finger 2 |
| | | | | RNF131 | |
| 1703 | 079N | Hs01937849_s1 | GPBAR1 | BG37, GPCR19, | G protein- |
| | | | | GPR131, | coupled bile |
| | | | | M- | acid receptor 1 |
| | | | | BAR, MGC40597, TGR5 | |
| 1704 | 079P | Hs00367579_m1 | CRLF3 | CREME9, | cytokine |
| | | | | CYTOR4, | receptor-like |
| | | | | FRWS, MGC20661, | factor 3 |
| | | | | p48.2 | |
| 1705 | 080N | Hs00201854_m1 | GCA | GCL | grancalcin, |
| | | | | | EF-hand |
| | | | | | calcium |
| | | | | | binding |
| | | | | | protein |
| 1706 | 080P | Hs00406762_m1 | PRKAR1B | PRKAR1 | protein |
| | | | | | kinase, |
| | | | | | cAMP- |
| | | | | | dependent, |
| | | | | | regulatory, |
| | | | | | type I, beta |
| 1707 | 081N | Hs00383944_m1 | C5orf62 | MGC117221, | chromosome |
| | | | | MGC126887, MGC126889, | 5 open |
| | | | | MST150, | reading frame |
| | | | | NID67 | 62 |
| 1708 | 081P | Hs00243205_m1 | SNURF | DKFZp686C0927, DKFZp686M12165, DKFZp761I1912, | SNRPN |
| | | | | DKFZp762N022, | upstream |
| | | | | FLJ33569, | reading |
| | | | | FLJ36996, | frame, small |
| | | | | FLJ39265, | nuclear |
| | | | | HCERN3, | ribonucleoprotein |
| | | | | MGC29886, PWCR, | polypeptide N |
| | | | | RT- | |
| | | | | LI, SM- | |
| | | | | D, SMN, SNRNP- | |
| | | | | N, SNURF- | |
| | | | | SNRPN | |
| 1709 | 082N | Hs02379634_s1 | CH25H | C25H | cholesterol |
| | | | | | 25- |
| | | | | | hydroxylase |
| 1710 | 082P | Hs02578922_gH | MT1G | MGC12386, | metallothionein |
| | | | | MT1, MT1K | 1G |
| 1711 | 083N | Hs02621289_g1 | TPT1 | FLJ27337, | tumor protein, |
| | | | | HRF, RP11- | translationally- |
| | | | | 290D2.1, | controlled 1 |
| | | | | TCTP, p02 | |
| 1712 | 083P | Hs00233287_m1 | IKBKB | FLJ33771, | inhibitor of |
| | | | | FLJ36218, | kappa light |
| | | | | FLJ38368, | polypeptide |
| | | | | FLJ40509, | gene |
| | | | | IKK- | enhancer in |
| | | | | beta, IKK2, | B-cells, kinase |
| | | | | IKKB, MGC131801, | beta |
| | | | | NFKBIKB | |
| 1713 | 084N | Hs00541730_m1 | CHST13 | C4ST3, MGC119278, | carbohydrate |
| | | | | MGC119279, | (chondroitin 4) |
| | | | | MGC119281 | sulfotransferase |
| | | | | | 13 |
| 1714 | 084P | Hs00371639_m1 | SNUPN | KPNBL, RNUT1, | snurportin 1 |
| | | | | Snurportin1 | |
| 1715 | 085N | Hs00384007_m1 | WSCD1 | KIAA0523 | WSC domain |
| | | | | | containing 1 |
| 1716 | 01HSK | Hs02338565_gH | RPL19 | DKFZp779D216, | ribosomal |
| | | | | FLJ27452, MGC71997 | protein L19 |
| 1717 | 02HSK | Hs01086912_m1 | HNRNPD | AUF1, AUF1A, | heterogeneous |
| | | | | HNRPD, | nuclear |
| | | | | P37, hnRNPD0 | ribonucleoprotein |
| | | | | | D (AU-rich |
| | | | | | element RNA |
| | | | | | binding |
| | | | | | protein 1, |
| | | | | | 37 kDa) |
| 1718 | 03HSK | Hs00828752_gH | RPS20 | FLJ27451, | ribosomal |
| | | | | MGC102930 | protein S20 |
| 1719 | 04HSK | Hs01631495_s1 | RPL26L1 | FLJ46904, | ribosomal |
| | | | | RPL26P1 | protein L26- |
| | | | | | like 1 |
| 1720 | 05HSK | Hs00254535_m1 | EIF4H | KIAA0038, | eukaryotic |
| | | | | WBSCR1, | translation |
| | | | | WSCR1 | initiation factor |
| | | | | | 4H |
| 1721 | 06HSK | Hs00371372_m1 | SON | BASS1, C21ORF50, | SON DNA |
| | | | | DBP- | binding |
| | | | | 5, FLJ21099, | protein |
| | | | | FLJ33914, | |
| | | | | HSPC310, | |
| | | | | KIAA1019, | |
| | | | | NREBP, | |
| | | | | SON3 | |
| 1722 | 07HSK | Hs00763191_s1 | YPEL5 | CGI-127 | yippee-like 5 |
| | | | | | (Drosophila) |
| 1723 | 08HSK | Hs01115161_m1 | DPP7 | DPP2, DPPII, | dipeptidyl- |
| | | | | QPP | peptidase 7 |
| 1724 | 10NSK | Hs00559413_m1 | ANXA7 | ANX7, RP11- | annexin A7 |
| | | | | 537A6.8, | |
| | | | | SNX, SYNEXIN | |
| 1725 | 11HSK | Hs00212868_m1 | PAIP2 | HSPC218, | poly(A) |
| | | | | MGC72018, | binding |
| | | | | PAIP2A | protein |
| | | | | | interacting |
| | | | | | protein 2 |
| 1726 | 12HSK | Hs00199284_m1 | RAB35 | H- | RAB35, |
| | | | | ray, RAB1C, | member RAS |
| | | | | RAY | oncogene |
| | | | | | family |
| 1727 | 13HSK | Hs00606477_m1 | CDC37 | P50CDC37 | cell division |
| | | | | | cycle 37 |
| | | | | | homolog (S. cerevisiae) |
| 1728 | 14HSK | Hs00194538_m1 | SRSF4 | SFRS4, SRP75 | serine/arginine- |
| | | | | | rich splicing |
| | | | | | factor 4 |
| 1729 | 15HSK | Hs00363236_m1 | AKIRIN2 | C6ORF166, | akirin 2 |
| | | | | FBI1, FLJ10342, | |
| | | | | dJ486L4.2 | |
| 1730 | 16HSK | Hs00229388_m1 | CCDC130 | MGC10471, | coiled-coil |
| | | | | SB115 | domain |
| | | | | | containing |
| | | | | | 130 |
| 1731 | 17HSK | Hs00272036_m1 | CD2BP2 | FWP010, | CD2 |
| | | | | LIN1, Snu40, | (cytoplasmic |
| | | | | U5- | tail) binding |
| | | | | 52K | protein 2 |
| 1732 | 18HSK | Hs00197056_m1 | TIMM23 | FLJ40725, | translocase of |
| | | | | FLJ56773, | inner |
| | | | | FLJ57459, | mitochondrial |
| | | | | FLJ79448, | membrane 23 |
| | | | | MGC71995, MGC87383, | homolog |
| | | | | RP11- | (yeast), translocase |
| | | | | 481A12.7, | of inner |
| | | | | TIM23, RP11- | mitochondrial |
| | | | | 592B15.7, | membrane 23 |
| | | | | bA592B15.7 | homolog B |
| | | | | | (yeast) |
| 1733 | 20HSK | Hs00193824_m1 | MED6 | NY-REN- | mediator |
| | | | | 28 | complex |
| | | | | | subunit 6 |
| 1734 | 21HSK | Hs00702452_s1 | NUDC | HNUDC, | nuclear |
| | | | | MNUDC, | distribution |
| | | | | NPD011 | gene C |
| | | | | | homolog (A. nidulans) |
| 1735 | 22HSK | Hs00220038_m1 | TMEM167B | AD- | transmembrane |
| | | | | 020, C1ORF119, | protein |
| | | | | FLJ90710 | 167B |
| 1736 | 23HSK | Hs00229455_m1 | URM1 | C9ORF74, | ubiquitin |
| | | | | MGC2668, | related |
| | | | | RP11- | modifier 1 |
| | | | | 339B21.4 | |
| 1737 | 24HSK | Hs00412682_m1 | UBXN4 | FLJ23318, | UBX domain |
| | | | | KIAA0242, | protein 4 |
| | | | | KIAA2042, | |
| | | | | UBXD2, | |
| | | | | UBXDC1, | |
| | | | | erasin | |
| 1738 | 25HSK | Hs00380814_m1 | FAM177A1 | C14ORF24, | family with |
| | | | | DKFZp686J1254, | sequence |
| | | | | FLJ38854 | similarity 177, |
| | | | | | member A1 |

TABLE 3
20 Positive and Negative Predictor Genes of GVHD Outcome and Exemplary Probes

| | | | | | | | | Minimal | Minimal |
|---|---|---|---|---|---|---|---|---|---|
| | | | | | | | | p-value | p-value |
| | | | | | | | P or N | precision- | standard |
| | | | | | | | predictor | weighted | heteroscedastic |
| Index | ProbeID | Accession no. | Gene name | Symbol | Synonyms | Probe sequence | gene | T-test | T-test |
| 1 | 380575 | NM_000978.3 | ribosomal protein L23 | RPL23 | MGC117346; rpL17; | TCCAGCAGTGGTCATTCG | N | 0.009966 | 0.001089 |
| | | | (RPL23), mRNA. | | MGC111167; MGC72008 | ACAACGAAAGTCATACCGT | | | |
| | | | | | | AGAAAAGATGGCG | | | |
| 2 | 940398 | NM_006360.3 | eukaryotic translation | EIF3M | FLJ29030; GA17; hfl-B5; | CAGACCCAGAGAAAAGTA | N | 0.013671 | 0.00275 |
| | | | initiation factor 3, subunit M | | eIF3m; PCID1; B5 | GTTGTCAGTCATAGCACAC | | | |
| | | | (EIF3M), mRNA. | | | ATCGGACATTTGG | | | |
| 3 | 990315 | NM_030752.2 | t-complex 1 (TCP1), | TCP1 | TCP-1-alpha; CCT-alpha; | GCAATGGTAAACCTCGAG | N | 0.001814 | 9.08E−05 |
| | | | transcript variant 1, mRNA. | | CCT1; D6S230E; CCTa | ACAACAAACAAGCAGGGG | | | |
| | | | | | | TGTTTGAACCAACC | | | |
| 4 | 1240136 | NM_199345.3 | phosphatidylinositol 4- | PI4KAP2 | FLJ44912; MGC31920 | GTGAGCCTGGGCCCTACA | P | 0.009399 | 0.000919 |
| | | | kinase, catalytic, alpha | | | TGGATGTGGTCGTCTCCC | | | |
| | | | polypeptide pseudogene 2 | | | TGGTCACTATCATG | | | |
| | | | (PI4KAP2), mRNA. | | | | | | |
| 5 | 1820482 | NM_004548.1 | NADH dehydrogenase | NDUFB10 | PDSW | CAGAGGCAGAGGATGCTG | N | 0.001628 | 0.000849 |
| | | | (ubiquinone) 1 beta | | | CAAGAGAGAAAAGCTGCA | | | |
| | | | subcomplex, 10, 22 kDa | | | AAAGAGGCCGCCGC | | | |
| | | | (NDUFB10), mRNA. | | | | | | |
| 6 | 1850288 | NM_014153.2 | zinc finger CCCH-type | ZC3H7A | HSPC055; ZC3HDC7; | GTTGGGGAAGAGGATAAG | N | 0.009361 | 5.94E−05 |
| | | | containing 7A (ZC3H7A), | | ZC3H7; FLJ20318; | GTTATATCTAGGACAACTC | | | |
| | | | mRNA. | | FLJ10027 | TTTGAGTTGGTCC | | | |
| 7 | 2940022 | NM_000712.3 | biliverdin reductase A | BLVRA | BVRA; BLVR | CTGAGAAGGAACTGGCTG | N | 0.001468 | 0.000119 |
| | | | (BLVRA), mRNA. | | | CTGAAAAGAAACGCATCCT | | | |
| | | | | | | GCACTGCCTGGGG | | | |
| 8 | 3370164 | NM_000701.6 | ATPase, Na+/K+ | ATP1A1 | MGC3285; MGC51750 | CGAAGTCAGAAAACTCATC | P | 0.006818 | 0.000591 |
| | | | transporting, alpha 1 | | | ATCAGGCGACGCCCTGGC | | | |
| | | | polypeptide (ATP1A1), | | | GGCTGGGTGGAGA | | | |
| | | | transcript variant 1, mRNA. | | | | | | |
| 9 | 3440400 | NM_020698.1 | transmembrane and coiled- | TMCC3 | KIAA1145 | GGGCAAACCCAAAGATGG | N | 0.019615 | 1.54E−06 |
| | | | coil domain family 3 | | | AAAGTGCTTGTTGGGTGG | | | |
| | | | (TMCC3), mRNA. | | | GTAAGCACCACCTG | | | |
| 10 | 3450148 | NM_170734.2 | brain-derived neurotrophic | BDNF | MGC34632 | ATGTACGTGGGGGATTCTT | N | 0.012771 | 1.75E−05 |
| | | | factor (BDNF), transcript | | | GACTCGGGTTAGTCTCTG | | | |
| | | | variant 6, mRNA. | | | GGGATGCAGAGCC | | | |
| 11 | 3780450 | NM_079837.2 | BTG3 associated nuclear | BANP | DKFZp761H172; | TTTCGTTTGAGTCCTGCTG | P | 0.012871 | 0.007059 |
| | | | protein (BANP), transcript | | FLJ10177; SMAR1; | TTGGTGTCGGAGCACGAG | | | |
| | | | variant 2, mRNA. | | SMARBP1; FLJ20538 | GGGAGGCACGGTG | | | |
| 12 | 4200575 | NM_014232.1 | vesicle-associated | VAMP2 | SYB2; VAMP-2; FLJ11460 | GCCCAGAGAGAGCTGTCC | P | 0.001982 | 0.000689 |
| | | | membrane protein 2 | | | TCTCATTGGGTGAACTGAT | | | |
| | | | (synaptobrevin 2) (VAMP2), | | | TGAGGAAGGGTCT | | | |
| | | | mRNA. | | | | | | |
| 13 | 4640689 | NM_001967.3 | eukaryotic translation | EIF4A2 | DDX2B; BM-010; EIF4A; | GGACCCTGTTGCTAAGCC | P | 0.004037 | 0.000412 |
| | | | initiation factor 4A, isoform | | EIF4F | CCAGCAAGCAATCCTAGG | | | |
| | | | 2 (EIF4A2), mRNA. | | | TAGGGTTTAATCCC | | | |
| 14 | 5220196 | NM_006565.2 | CCCTC-binding factor (zinc | CTCF | — | ATGTAGCAGAATGGCACC | P | 0.007622 | 0.000468 |
| | | | finger protein) (CTCF), | | | CAGACCACTGCCCACCAG | | | |
| | | | mRNA. | | | TGACGGACATGCAC | | | |
| 15 | 5870632 | NM_004800.1 | transmembrane 9 | TM9SF2 | P76; MGC117391; | CAGTGTGGTGAAGGTTGA | N | 0.004099 | 0.001709 |
| | | | superfamily member 2 | | FLJ26287 | CTGAAGAAGTCCAGTGTG | | | |
| | | | (TM9SF2), mRNA. | | | TCCAGTTAAAACAG | | | |
| 16 | 6290392 | NM_005839.3 | serine/arginine repetitive | SRRM1 | SRM160; 160-KD; | CAACTTTCAGAGCCTCTTG | P | 0.007348 | 0.000539 |
| | | | matrix 1 (SRRM1), mRNA. | | POP101; MGC39488 | TATTTGGAAGGCTGGAAG | | | |
| | | | | | | GGCCCAGACTTTG | | | |
| 17 | 6380008 | NM_025209.2 | enhancer of polycomb | EPC1 | Epl1; DKFZp781P2312 | ACACAGTAGCGATGGAGG | P | 0.008241 | 0.00013 |
| | | | homolog 1 (Drosophila) | | | TGACGTAGCTTCCTCCGA | | | |
| | | | (EPC1), mRNA. | | | GTGGAACTGCAGCC | | | |
| 18 | 6380427 | NM_202468.1 | GIPC PDZ domain | GIPC1 | IIP-1; TIP-2; GLUT1CBP; | CCCTCCCTGTGGAGCCTG | P | 0.0106 | 0.003695 |
| | | | containing family, member 1 | | C19orf3; RGS19IP1; | TTACCTCCGCATTTGACAC | | | |
| | | | (GIPC1), transcript variant | | Hs.6454; SYNECTIIN; | GAGTCTGCTGTGA | | | |
| | | | 3, mRNA. | | MGC15889; NIP; | | | | |
| | | | | | MGC3774; SEMCAP; GIPC | | | | |
| 19 | 6580553 | NM_005688.2 | ATP-binding cassette, sub- | ABCC5 | MOAT-C; pABC11; ABC33; | GTTTGGTGTGTTCCCGCAA | P | 0.030046 | 0.000792 |
| | | | family C (CFTR/MRP), | | MRP5; SMRP; EST277145; | ACCCCCTTTGTGCTGTGG | | | |
| | | | member 5 (ABCC5), | | DKFZp686C1782; MOATC | GGCTGGTAGCTCA | | | |
| | | | transcript variant 1, mRNA. | | | | | | |
| 20 | 7210128 | NM_024408.2 | Notch homolog 2 | NOTCH2 | hN2; AGS2 | AGCCATAGCTGGTGACAA | N | 0.015967 | 0.008984 |
| | | | (Drosophila) (NOTCH2), | | | ACAGATGGTTGCTCAGGG | | | |
| | | | mRNA. | | | ACAAGGTGCCTTCC | | | |

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., compound structures) are an example of a genus of equivalent or similar features.

As used herein, the singular forms “a”, “and,” and “the” include plural referents unless the context clearly indicates otherwise. Thus, for example, reference to “a first, second, third, fourth, fifth, etc. predictor gene” or a “positive or negative predictor gene” includes a plurality of such first, second, third, fourth, fifth, etc., genes, or a plurality of positive and/or negative predictor genes.

All applications, publications, patents and other references, GenBank citations and ATCC citations cited herein are incorporated by reference in their entirety. In case of conflict, the specification, including definitions, will control.

As used herein, all numerical values or numerical ranges include integers within such ranges and fractions of the values or the integers within ranges unless the context clearly indicates otherwise. Thus, to illustrate, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

Reference to a number with more (greater) or less than includes any number greater or less than the reference number, respectively. Thus, for example, a reference to less than 30,000, includes 29,999, 29,998, 29,997, etc. all the way down to the number one (1); and less than 20,000, includes 19,999, 19,998, 19,997, etc. all the way down to the number one (1).

Reference to a range or series of ranges includes integers within the ranges, subranges, and combinations of the series of ranges. For example, a range of 5 to 10 therefore includes 1, 2, 3, 4, 5, 6, 7, 8, 9, 10, and a range of 5 to 10 therefore includes 5 to 7, 5 to 8, 6 to 8, 5 to 9, 7 to 9, 5 to 10, etc. Reference to a series of ranges includes combinations of the upper and lower end of the ranges. For example, reference to a series of ranges from 1 to 10, to 20, 20 to 30, 30 to 40, 40 to 50, 60 to 70, 70 to 80, 80 to 90, 90 to 100, includes ranges from 5-20, 5-50, 5-100, 20-50, 20-100, 30-50, 30-100, 40 to 60, 40 to 70, 40 to 80, etc. and, so forth.

The invention is generally disclosed herein using affirmative language to describe the numerous embodiments. The invention also includes embodiments in which subject matter is excluded, in full or in part, such as substances or materials, method steps and conditions, protocols, or procedures. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

### Example 1

This example includes a description of materials and methods.

Sample Sources:

All 122 pre-transplant, frozen (liquid nitrogen) donor PBMC (peripheral blood mononuclear cells) samples and corresponding recipient GVHD histories were obtained under contract from the repository of frozen transplant donor blood samples and informational database of the NMDP (National Marrow Donor Program). All of the 122 HCTs examined correspond to HLA 10/10 matched unrelated donor transplantations, and originated from a total of 47 different transplant centers throughout the U.S. (Table 4). The HCTs examined were used for the treatment of NMDP-selected patients with ALL, AML, CML, or MDS. These 122 samples were analyzed and from these samples, exemplary positive and negative GVHD predictor genes are listed in Table 1 (RNA 1538).

The 6 different GVHD outcome Groups (capital “G”) are relatively evenly distributed for each center. This provides a highly diverse HCT sample source population, which will eliminate most potential biases, if any, of transplant clinical center-source sample processing and clinical outcome attribution.

TABLE 4
Centers

| | | Transplant Center (TC) | | | | | | | | | | | | | | | | | | | | | | | |
|---|---|---|---|---|---|---|---|---|---|---|---|---|---|---|---|---|---|---|---|---|---|---|---|---|---|
| | | 12 | 43 | 38 | 53 | 4 | 21 | 17 | 1 | 23 | 48 | 0 | 14 | 8 | 10 | 18 | 19 | 20 | 28 | 33 | 36 | 42 | 46 | 52 | 2 |
| No. donors | | 19 | 8 | 7 | 7 | 6 | 6 | 5 | 4 | 4 | 4 | 3 | 3 | 2 | 2 | 2 | 2 | 2 | 2 | 2 | 2 | 2 | 2 | 2 | 1 |
| per TC | | | | | | | | | | | | | | | | | | | | | | | | | |
| No. donors | G1 | — | 1 | 2 | 1 | 2 | 2 | — | 2 | 2 | — | — | 1 | 2 | 1 | 1 | — | 1 | — | — | 2 | — | — | — | 1 |
| per | G2 | 1 | 3 | 3 | — | 2 | 2 | 1 | — | — | — | 1 | — | — | — | 1 | — | — | — | — | — | — | 1 | 2 | — |
| GVHD | G3 | 3 | — | 1 | — | — | — | — | 1 | 1 | 3 | 1 | — | — | — | — | — | 1 | 1 | 1 | — | 1 | 1 | — | — |
| group | G4 | 6 | 3 | — | 1 | — | — | 3 | — | — | — | — | 1 | — | — | — | — | — | 1 | — | — | — | — | — | — |
| | G5 | 2 | — | — | 2 | 1 | — | — | 1 | 1 | — | 1 | — | — | — | — | 1 | — | — | — | — | 1 | — | — | — |
| | G6 | 7 | 1 | 1 | 3 | 1 | 2 | 1 | — | — | 1 | — | 1 | — | 1 | — | 1 | — | — | 1 | — | — | — | — | — |

| | | Transplant Center (TC) | | | | | | | | | | | | | | | | | | | | | | |
|---|---|---|---|---|---|---|---|---|---|---|---|---|---|---|---|---|---|---|---|---|---|---|---|---|
| | | 3 | 5 | 6 | 7 | 11 | 15 | 16 | 22 | 25 | 26 | 27 | 29 | 32 | 34 | 35 | 37 | 39 | 40 | 41 | 44 | 45 | 49 | 51 |
| No. donors | | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 |
| per TC | | | | | | | | | | | | | | | | | | | | | | | | |
| No. donors | G1 | — | — | 1 | — | — | — | 1 | — | — | — | 1 | 1 | 1 | — | — | — | — | — | — | — | — | — | — |
| per | G2 | — | — | — | — | — | — | — | — | — | — | — | — | — | — | — | — | 1 | — | 1 | — | — | — | 1 |
| GVHD | G3 | — | — | — | 1 | 1 | — | — | — | 1 | 1 | — | — | — | — | — | — | — | — | — | — | 1 | — | — |
| group | G4 | — | — | — | — | — | — | — | 1 | — | — | — | — | — | — | 1 | — | — | — | — | — | — | — | — |
| | G5 | — | — | — | — | — | 1 | — | — | — | — | — | — | — | 1 | — | — | — | 1 | — | 1 | — | 1 | — |
| | G6 | 1 | 1 | — | — | — | — | — | — | — | — | — | — | — | — | — | 1 | — | — | — | — | — | — | — |

Patient GVHD-Related Disease Outcome Defined Groups:

GVHD outcomes for each transplantation are divided into six clinically relevant groups, named Group 1 through Group 6. These outcome groups cover several combinations of acute grades 3 or 4 (most intense and life-threatening) and acute grades 1 or 2 (less severe and occasionally considered mild) GVHD, and/or with extensive chronic GVHD (Table 5).

TABLE 5
GROUPS

| GVHD | | | | | | |
|---|---|---|---|---|---|---|
| group | G1 | G2 | G3 | G4 | G5 | G6 |
| Observed | no | no | grade 1&2 | grade 1&2 | grade 3&4 | grade 3&4 |
| GVHD | acute GVHD & | acute GVHD & | acute GVHD & | acute GVHD & | acute GVHD & | acute GVHD & |
| | no | extensive | no | extensive | no | extensive |
| | chronic GVHD | chronic GVHD | chronic GVHD | chronic GVHD | chronic GVHD | chronic GVHD |
| Total | n = 26 | n = 20 | n = 20 | n = 17 | n = 15 | n = 24 |
| donors | | | | | | |

In-Laboratory Selection of Blood-Derived Specific T-Lymphocytes for RNA Expressionanalysis:

CD4+ T-cells were separated from donor PBMC frozen blood samples using commercially available magnetic microbeads technology (Miltenyi Corp.), conducted under contract with a commercial laboratory (Southern Research Institute (SRI), Birmingham, Ala.). At another contract laboratory, RNA was subsequently extracted from purified CD4+ cells using the commercially available RNeasy kit (Qiagen).

For each of the resulting 122 donor RNA samples, gene expression (i.e., intra-cellular RNA abundance) was assayed for ˜20,000 genes (as represented by 48,803 human genome probes, each replicated through ˜20 independent technical measurements for robust signal averaging) using a commercially available Illumina HT-12 BeadArrays v3.0 microarrays (Illumina Corp.) (Illumina mRNA Expression Analysis, Customer Solutions, IIlumina, Inc., San Diego, Calif., esp. section IIlumina Whole-Genome Gene Expression BeadChips, pp. 5-7). The RNA-extractions and quantitative gene expression measurements on IIlumina microarrays and numerical digitization of the in-lab measurements were conducted under contract (Expression Analysis Inc. (EA), Durham, N.C.).

### Example 2

This example includes a description of data transformation for mathematical and numerical stabilization and background reduction.

In general, variance stabilization is intended to mathematically and statistically properly mitigate common phenomenon for many different kinds of real data: The variance, or standard deviation, of measurements inherently increases with measurement level rather than being essentially independent of level. It is desirable statistically to mitigate, or even remove, such “level-dependent” variance or standard deviation by appropriate theoretically or empirically justified mathematical transformations having certain acceptable properties statistically (Durbin, et al., Bioinformatics, 18, Suppl. 1, 5105-5110 (2002)).

Variance stabilization is called for primarily because most standard parametric tests (e.g., T-tests) and indeed some non-parametric tests (e.g., rank-based statistical tests), theoretically and practically assume that the variance, or standard deviation, of a set of measurements does not depend on the mean of such measurements. Analogously, this also applies for the variance or standard deviation or measurement error of a single measurement and the level of that single measurement. To the extent that variance or standard deviation of measurements depend on the level of the measurements, is the extent to which, practically speaking, statistical tests based on level-independent variance or standard deviation is less trustworthy. Hence, if there is substantial (relative, say, to differences between means of certain germane subsets of data) level-dependence of variance or standard deviation, then it is recommended to apply a variance stabilization mathematical \numerical transformation of the data before doing the statistical tests (Sheskin, David J. Handbook of Parametric and Nonparametric Statistical Procedures, 3^{rd} Edition, Chapman & Hall/CRC Press, Boca Raton, Fla., 2004, esp. pp. 404-409).

Mathematical\Numerical Stabilization of Quantitative Measurements Using Specific VST (“Variance Stabilization Transformation”):

Before application of a customized computational pre- or post-processing of quantitative gene expression data that identified positive and negative genes predictive of HCT that induces GVHD or not, Illumina microarray gene expression data was background-subtracted using the conventional manufacturer-supplied Illumina Bead Studio software, as performed by Expression Analysis Inc. All microarray-derived gene expression data, sample by sample, was then subjected computationally to a customized implementation of Illumina measurements Variance Stabilizing Transformation (VST), then linearly resealed robustly to a maximum of ˜4.5, and then quantile normalized. (Lin, et al., Nucleic Acids Research 36(2):ell, 1-9 (2008)) for the background mathematical statistics of VST as applied to Illumina BeadArray microarray data).

Certain mathematical details in (Lin, et al., Nucleic Acids Research 36(2):ell, 1-9 (2008) were updated as described in detail below, and subsequently validated technically. The VST refined mathematics and implementation in customized Matlab programming language (The MathWorks, Inc., 3 Apple Hill Dr., Natick, Mass. 01760) was developed and implemented.

In particular, an IIlumina BeadArray-specific mathematical\statistical\numerical VST (Lin, et al., Nucleic Acids Research 36(2):ell, 1-9 (2008), with refinements) was applied sample-by-sample (i.e., for each donor HCT separately) to the Illumina platform-derived gene expression numerical measurements. This “pre-processing” mathematical \statistical \numerical data treatment step operating on the gene expression measurements was applied before outcome predictive data analysis.

VST specifically designed for Illumina platform-derived gene expression data is not common and not widely used, though it is established in the scientific literature (Durbin, et al., Bioinformatics, 18, Suppl. 1, S105-S110 (2002); Dunning, et al., BMC (Biomed Central) Bioinformatics 9, #85, doi:10.1186/1471-2105-9-85, 1-15 (2008)). From the IIlumina-oriented published literature (Illumina mRNA Expression Analysis, Customer Solutions, IIlumina, Inc., San Diego, Calif., esp. section Illumina Whole-Genome Gene Expression BeadChips, pp. 5-7; Dunning, et al., BMC (Biomed Central) Bioinformatics 9, #85, doi:10.1186/1471-2105-9-85, 1-15 (2008)) and from a detailed scrutiny of the Illumina data from this study, there is a profound tendency of each gene expression measurement's inherent technical standard deviation or technical “error”, i.e., Illumina platform's provided so-called Bead Standard Error (Illumina mRNA Expression Analysis, Customer Solutions, Illumina, Inc., San Diego, Calif., esp. section IIlumina Whole-Genome Gene Expression BeadChips, pp. 5-7; Dunning, et al., BMC (Biomed Central) Bioinformatics 9, #85, doi:10.1186/1471-2105-9-85, 1-15 (2008)), to increase substantially with magnitude of the expression measurement. Accordingly, VST was applied to the Illumina data from this study, and because of this study's use of LDA (linear discriminant analysis) and T-tests.

A modification of the state-of-the-art versio of VST for IIlumina, as published by Lin, et al. (Lin, et al., Nucleic Acids Research 36(2):ell, 1-9 (2008)), was developed and applied to the gene expression data.

Numerical Data Tabular Arrangement:

In the following description the in-laboratory Illumina-platform generated data is arranged as a 2-dimensional table or matrix: Samples, i.e., donor HCTs or control, refer to columns, and on-platform (i.e., IIlumina BeadArray) specifically defined molecular probes refer to rows. A given row therefore represents data associated with the same probe, and across all columns, i.e., across all HCT samples and controls. A given column represents a given sample, i.e., a given donor HCT or control, and each row represents a measurement value associated with a specific probe, row-by-row respectively, and in the same row-wise ordering for every sample. E.g., for the Illumina HT-12 BeadArray version 3.0 employed in studies, there are 48803 probes; hence 48803 rows (before any row-wise sub-selections might be made). The number of columns involved depends on the number of HCT samples.

Illumina Array Provides 3 Numerical Quantities with Gene Expression Measurement Signal:

Four separate (though interrelated) kinds of measurement values are provided by the Illumina platform: gene expression signal, bead standard error, average number of beads involved in the signal measurement, and Illumina-defined and computed signal “detection p-value.” The bead standard error, average number of beads, and signal detection p-value are associated with each and every gene expression signal measurement (BeadStudio Gene Expression Module v3.2 User Guide, Part #11279596 Rev. A, IIlumina, Inc., San Diego, Calif., esp., Detection P-value section, Normalization and Differential Analysis, Ch. 4.). In the following description, gene expression measurement is referred to as signal; bead standard error is abbreviated as bead_stderr (or similar such names, lower- or upper-case); average number of beads is abbreviated as avg_nbeads (or similar such names, lower- or upper-case); and detection p-value is abbreviated as detection p-value (or similar such names, lower- or upper-case).

Background Subtraction—Instrumental and Subsequent Contexts:

In the expression studies, signal refers to raw signal numerical values provided by the Illumina platform, minus the so-called numerically estimated instrumental “local background fluorescence” as assessed and computed by the Illumina platform for each probe of any given sample. I.e., in the studies, signal starts as raw signal minus Illumina platform-provided “background” subtraction. Also in the studies, the signals provided by the Illumina platform are not “normalized” by the Illumina platform.

In the course of the mathematical, statistical, and numerical computational processing for VST, negative or near-zero signal values are themselves considered as gene expression measurement “background” to be accounted for, and adjusted for, within the VST procedure. This will be made explicit and clear later in the description.

Tabular Arrangement of Illumina-Provided Quantities:

As stated above, there is a numerical data matrix (probes in rows, samples in columns) for signal. Also, there is such an analogous, row-wise and column-wise in register, numerical data matrix for each of the 3 non-signal Illumina-provided numerical measurements: bead standard error of the signal measurement, average number of beads involved in the signal measurement, and the Illumina-defined and provided “detection p-value”. Thus at the fundamental level, there are always these 3 kinds of laboratory instrument-level quantities associated with a given signal value for a given probe for a given sample. This triple of information is harnessed and exploited in the VST for Illumina data (Lin, et al., Nucleic Acids Research 36(2):ell, 1-9 (2008)), and in the customized modification and implementation of VST for the Illumina data.

The data is transformed before outcome prediction data analysis is carried out. VST is applied separately to each individual sample separately across all probes. Example real results for sample-by-sample compute VST parameters c1, c2, and c3 for each of 48 samples across 48803 probes are shown below in Table 6. Even so, in the following description, explanations of mathematical and statistical methods and computational procedures do not focus narrowly on individual samples per se one-by-one as examples. Thus, in the below, often the data and plots will be referred to in terms of all probes (48803 probes) and all HCT samples (e.g., 48 samples for one particular stage of the studies). Such will then comprise, e.g., 48803 by 48=2,342,544 numerical values of signal, of corresponding bead_stderr, of corresponding avg_nbeads, and of corresponding detection p-values.

Signal Histogram:

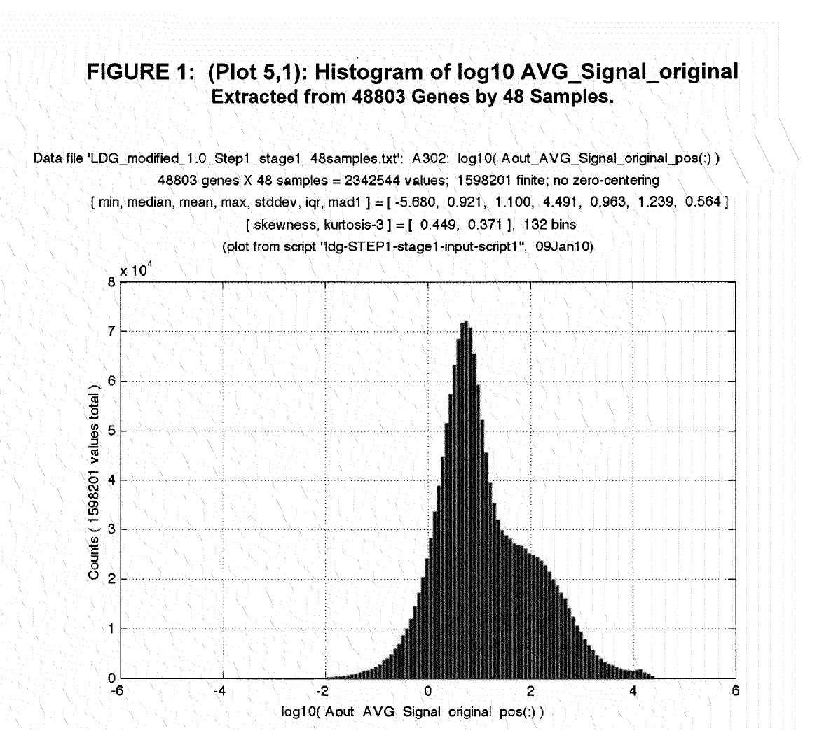

A representative unsmoothed histogram of 48803 probes by 48 samples Illumina signal values is shown in FIG. 1 (Plot 5,1). Due to the range of possible raw Illumina “background”-subtracted values being from about several hundred negative to about 40,000 positive, the empirical distribution is visualized more clearly when the logarithm base ten (“log 10”) of the signal values are histogrammed. Of course, due to logarithm, only positive raw signal values can be represented in such a histogram. FIG. 1 (Plot 5,1) is a fundamental empirical distributional view of the all the positive signal values. Note that this log 10(positive signal) histogram is not strictly single Gaussian-like (i.e., has a discernible broad shoulder to the right of the main peak).

Bead Standard Error, Average Number of Beads, Signal, and Histograms:

An advantage of the IIlumina BeadArray technology for measuring many thousands of gene expressions per sample is the provision of bead standard error (Dunning, et al., BMC (Biomed Central) Bioinformatics 9, #85, doi:10.1186/1471-2105-9-85, 1-15 (2008); BeadStudio Gene Expression Module v3.2 User Guide, Part #11279596 Rev. A, Illumina, Inc., San Diego, Calif., esp., Detection P-value section, Normalization and Differential Analysis, Ch. 4.). Due to the physical nature of the Illumina platform, bead standard error can be considered physically and statistically as the standard error (at the instrument level) of a given signal measurement. That is, bead standard error of a given probe of a given sample can be considered as a conventional error bar half-width as standard error (i.e., as measurement standard deviation divided by the square root of the number of replicates of the measurement, and which for the Illumina platform is bead_stderr=measurement standard deviation divided by sqrt(avg_nbeads) involved in the measurement) around the corresponding signal value (Durbin, et al., Bioinformatics, 18, Suppl. 1, S105-S110 (2002); Dunning, et al., BMC (Biomed Central) Bioinformatics 9, #85, doi:10.1186/1471-2105-9-85, 1-15 (2008)). The Illumina platform conducts separate measurements of individual Illumina “beads” to constitute ultimately a reported signal value and a bead standard error along with average number of beads involved in obtaining the reported signal and bead standard error (BeadStudio Gene Expression Module v3.2 User Guide, Part #11279596 Rev. A, Illumina, Inc., San Diego, Calif., esp., Detection P-value section, Normalization and Differential Analysis, Ch. 4.; Lin, et al., Nucleic Acids Research 36(2):ell, 1-9 (2008)). Typically as observed in these studies, there are about 20 separate beads on average being involved in a given gene probe's signal measurement. Because of these fundamentals, a given signal is really an individual bead-wise average signal; hence, “signal” is actually inherently an “average signal.” In the following description, the words signal and average signal are interchangeable for the same quantity, i.e., the signal, i.e., the average signal, reported by the Illumina platform for a given probe for a given sample. More specifically, the standard deviation of an Illumina-provided signal measurement (i.e., of “average signal”) is the square root of the average number of beads involved in the measurement (i.e., sqrt(avg_nbeads) times the corresponding reported bead standard error (Durbin, et al., Bioinformatics, 18, Suppl. 1, S105-S110 (2002); Lin, et al., Nucleic Acids Research 36(2):ell, 1-9 (2008)). Though vaguely Gaussian-like distributionally, bead standard error increases markedly, substantially, with increasing signal. This is the fundamental reason that a well-behaved and implementable variance stabilization transformation be applied to the Illumina data. This phenomenon calls for application of VST to the data BEFORE subsequent GVHD outcome-predictive analysis and discovery to mitigate such marked level-dependence of bead standard error, or standard deviation, or variance on signal level.

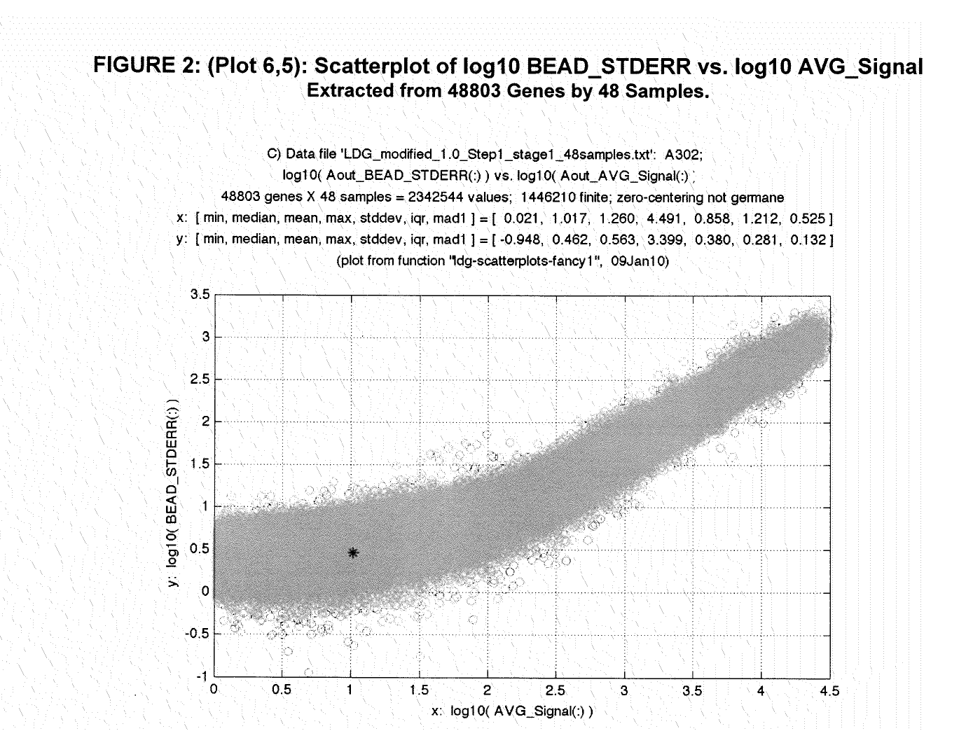

In further support of the above findings, the observed signal level-dependence of bead standard error (and hence of essentially variance too) is shown clearly and dramatically in FIG. 2 (Plot 6,5). FIG. 2 is a scatterplot of log 10(bead_stderr) vs. log 10(positive signal). Clearly, bead_stderr whether as is, or in log 10 units as in FIG. 2, is not constant vs. signal. Comments on Illumina detection p-values germane to VST, and why reported Illumina detection p-values are employed in a highly limited way: Implementation of VST employs an “approximate signal detection high-quality” threshold by requiring signals employed in computing the VST parameters per se from data to be based on Illumina platform high-quality signal detection (details below). This is an additional step added to the published version of VST (Lin, et al., Nucleic Acids Research 36(2):ell, 1-9 (2008)) to assure that the data-derived computed VST parameters are based on technically reliable signal measurements not near instrumental background noise even though subsequently the data-derived parametrically so-defined VST is applied to all the data. That is, the reliability of the computed VST is assured in principle by very conservatively basing the data-dependent computed VST parameters c₁, c₂, c₃ per se (see below) on sets of signal values for which the lesser technical quality signals (technically according to IIlumina platform provided detection p-value) are omitted from the VST parameter calculations per se. The algorithm employed by the Illumina platform in generating the reported detection p-values is complex. (See Illumina mRNA Expression Analysis, Customer Solutions, IIlumina, Inc., San Diego, Calif., esp. section Illumina Whole-Genome Gene Expression BeadChips, pp. 5-7, especially BeadStudio Gene Expression Module v3.2 User Guide, Part #11279596 Rev. A, Illumina, Inc., San Diego, Calif., esp., Detection P-value section, Normalization and Differential Analysis, Ch. 4.) In practice, empirical distributional properties of detection p-values can be computed from ensembles of actual Illumina data to guide practical judgment concerning the use of reported detection p-values for specific purposes, particularly in choosing to omit the technically less reliable signals from certain calculations.

Therefore, when setting a detection p-value limit of <0.5 for data employed in VST parameters per se calculations, those calculations are based on the technically most reliable 60% of the data, i.e., the technically most reliable majority of the signal data, regardless of experimental or biological interpretations that might be associated with such data. Hence, the data-dependent computationally derived VST parameters' values per se are reliable values in being derived from technically very reliable data. Detection p-values>about 0.5 are associated predominantly with signals near zero and indeed especially with negative signals, i.e., with signals whose level is essentially equivalent to low-level background noise. (Note: Biologically, such genes can be interpreted as being either at, or below, reasonable instrumental detection limits; hence more or less reliably “off” in gene expression. A measured gene expression signal that is near instrumental detection limit (and hence its numerical value is small but not reliable as a quantitative number differing from “noise”) can very well be reliably “off” in gene expression when interpreted biologically. This is not an artifact, but a fact of physically reality: Measured quantities whose values are near instrumental detection limit, hence not reliable numerical values per se, are still very reliably “absent” in physical, chemical, or biological interpretation.)

Hence, reported Illumina detection p-values are employed only in highly conservative approaches to calculating data-dependent VST parameters per se, i.e., by basing such calculations only on the majority of data unlikely to be near instrumental background “noise” in signal value.

Illumina measurement data needed specifically to compute VST:

Considerations of Background Signal:

VST as published by Lin, et al., Nucleic Acids Research 36(2):ell, 1-9 (2008) is based on a widely accepted, long-existing error-model or “noise” model from analytic chemistry for laboratory quantitative instrumentation (Durbin, et al., Bioinformatics, 18, Suppl. 1, S105-S110 (2002)), as instrumentally measured signals generally are corrupted inevitably, to very small, or sometimes to large, degrees by a combination of so-called additive and multiplicative noise.

Accordingly, low-level signals that can be considered reliably as being dominated by “background noise” are called “background signal”, and can be defined practically and operationally as Illumina reported signals for which reported detection p-values are>about 0.5. Hence, signals for which Illumina reported detection p-values are>0.5 as empirically defined “background signal.”

Considerations of “Background Signal” Variance Specifically for VST:

As mentioned above, very reliably signal standard deviation in general is stddev=sqrt(avg_nbeads)*bead_stderr. Hence, very reliably signal variance in general is stddev̂2=avg_nbeads*bead_stderr̂2. “Background signal” variance is thus computed as the square of stddev, and where “background signal” stddev is computed from avg_nbeads and bead_stderr.

VST Parameter c3 is Computed from “Background Signal” Variance:

By definition of the VST error model in Lin, et al., Nucleic Acids Research 36(2):ell, 1-9 (2008), data-derived VST parameter c3 is computed as the arithmetic mean of the “background signal” variance, i.e., signals for which detection p-value is >0.50.

Because the observed distribution of “background signal” variance is skewed rightward towards larger variance, the mean somewhat over-estimates the central tendency of “background signal” variance, i.e., mean is about 10% greater than median. In practical terms, it is safer and more conservative to slightly over-estimate “background signal” variance than to under-estimate it. Thus, it is good in practice to employ the mean rather than median in the numerical estimation of “background signal” variance for VST parameter c3. The c₃ calculations are implemented this way.

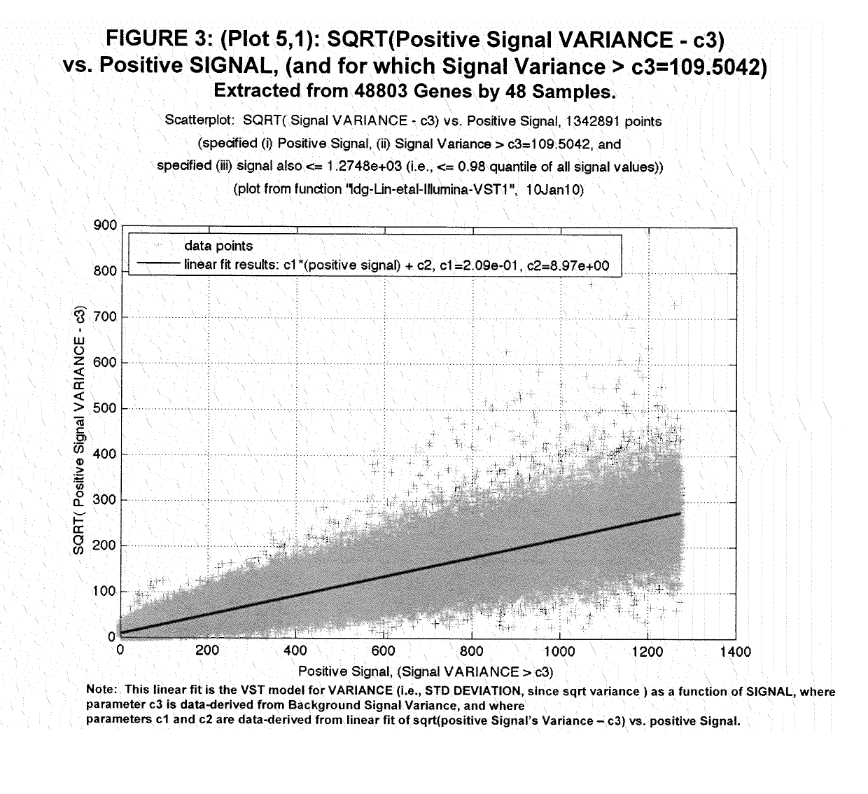

Algorithmically implementing another modification of the published VST procedure (Lin, et al., Nucleic Acids Research 36(2):ell, 1-9 (2008)) is to omit from the c1 and c2 calculations the largest 2% of the signals. In practice, the 98 percentile of observed signal values is less than about 1500 in raw signal value, e.g., the 98 percentile signal is about 1200 for the 48803 genes by 48 samples ensemble, i.e., only about 2% of the signals from the Illumina platform have observed values>about 1500. FIG. 3 (Plot 5,1) is important: (1) It shows empirically that the vast majority of Illumina raw signal data occurs at levels less than about 1500 even though there are many signals at the multiple tens of thousands level; (2) for the vast majority of signals, i.e., 98% of signals (because the largest 2% were omitted from the VST c1 and c2 parameters calculations), there is still clear and marked dependence of standard deviation or variance with signal level; and (3) it is precisely the data such as that represented in FIG. 3 (Plot 5,1) that is employed in the calculation of the VST data-dependent parameters c1 and c2 for each sample separately.

Calculation of VST Data-Dependent Parameters c1 and c2:

The procedure follows Lin, et al., Nucleic Acids Research 36(2):ell, 1-9 (2008), apart from introducing restriction to the smallest 98% of signal data going into the calculations. For a given set of data on which VST is computed, omitted from the c1 and c2 calculation are the largest 2% of signals. The implemented procedure is then: After c3 is computed as the “background signal” average variance as described above, consider (x,y)-pairs of data for which y is the sqrt(signal variance-c3) and for which signal variance exceeds c3, and x is the corresponding signal value. I.e., consider only those (x,y) for which y is positive and whose variance exceeds c3, and for which also x is less than the 98 percentile signal value. Then compute a linear fit of form y=c1*x+c2 to the set of (x,y) data points. I.e., c1 is defined as the slope of the linear fit, and c2 is defined as the y-intercept of the linear fit. Hence, numerical values for the data-dependent VST parameters c1 and c2 are so obtained from the (x,y) data points. The computed fitted line in FIG. 3 (Plot 5,1) has, e.g., slope c1=about 0.2 and y-axis intercept c2=about 9.0.

Variance Stabilizing Transformation (VST) Equation as a Formula:

VST is a specific, three-parameter, nonlinear function f operating upon Illumina platform-provided signal data. Function f transforms any signal value (not log signal value) to a new signal value t. I.e., VST in practice and in implementation is simply t=f(s; c1, c2, c3), where c1, c2, and c3 are numerical constants derived from a given set of Illumina data upon which VST is to be applied. This exposition and plots employ an example 48803 genes by 48 samples ensemble of real data. However, in actual practice, VST is applied to each sample separately, one sample at a time across all 48803 gene probes. In actual practice, 48 samples would require 48 separate applications of VST, each sample-wise instance of which would require its own calculation of the three required data-dependent constants c1, c2, and c3. (See Table 6 for example actual c1, c2, c3 numerical results for each of 48 samples, and for each sample compute across 48803 probes.) Relative to the data matrices describe above, VST is applied separately to each column: In deriving sample-specific values for constants c1, c2, and c3, and subsequently in transforming all the signal values s of the given column, i.e., given sample, by application of the VST function t=f(s; c₁, c₂, c₃) to the given column of data.

The fundamental VST equation for transforming raw gene expression signal s to corresponding variance stabilized transformed signal t=f(s; c₁, c₂, c₃), involving data-derived numerical values of parameters c₁, c₂, c₃ Lin, et al., Nucleic Acids Research 36(2):ell, 1-9 (2008):

$$t = f ( s ; {c}_{1} , {c}_{2} , {c}_{3} ) = \frac{1}{{c}_{1}} arcsinh ( \frac{{c}_{1} s + {c}_{2}}{\sqrt{{c}_{3}}} )$$

and where output t is the so-called variance stabilized transformed (i.e., “VST'ed”) signal; and where inputs arcsin h(z) is the conventional mathematical inverse hyperbolic sine function of real (not complex) negative, zero, or positive argument z; is raw platform-determined signal (not log-transformed, and with or without platform-determined instrumental background subtracted) and which can be negative, zero, or positive; and c₁, c₂, c₃ are the mathematically well-defined but data-derived across a sample or an ensemble of samples S numerical constants of the VST, all of which are positive. (Note: Mathematically c₃ can be zero and in which case the algebraic form of argument z changes; however, in practice c₃, is always positive because physically from the instrument, a signal's variance, whether from bead_stderr or from any other quantitation of physical variance, is never zero.)

For any Given Gene Probe for any Given Sample or Ensemble of Samples S:

c₃ is the arithmetic mean of the data-derived variances of signals s for which signals s are considered “background signals”, computed across most or all gene probes for a given sample or ensemble of samples; c₁ and c₂ are respectively the slope and y-intercept of a linear fit of (x,y) data y vs. x across most or all gene probes for a given sample (i.e., down a column, or along an ensemble of many columns, in the matrix of data described above), and where x is positive signal s for which the variance of s (denoted vans)) exceeds the already computed c₃ for the given sample or ensemble of samples, and where hence y=\sqrt{var(s)−c₃} (note: therefore y is always positive), and var(s) is the bead_stderr-derived, or other physically-derived variance, of signal s. Typically, Illumina platform bead-level technical variance of signal, denoted as var(s), is bead_stderr-derived, i.e., var(s)=stddev²(s)=((squareroot(avg_nbeads)) (bead_stderr))² (Durbin, et al., Bioinformatics, 18, Suppl. 1, S105-S110 (2002); BeadStudio Gene Expression Module v3.2 User Guide, Part #11279596 Rev. A, Illumina, Inc., San Diego, Calif., esp., Detection P-value section, Normalization and Differential Analysis, Ch. 4.) which of course also =(avg_nbeads) (bead_stderr)². The former formula is intended to be implemented to compute var(s) numerically since it is a little better-behaved with respect to numerical precision than the latter. Either formula implementation is acceptable, however.

Arcsin h(z)=ln(2z)+(1/(2*2*z²)−1*3/(2*4*4*z⁴)+1*3*5/(2*4*6*6*z⁶)- . . . , for |z|>1, and where ln(z) is the conventional notation for natural logarithm, i.e., logarithm base e.

A real advantage and benefit to gene expression numerical analysis generally is to adopt the arcsin h function to replace the logarithm function in the needed application of numerical transformations of gene expression signal data. Also, note that arcsin h(z) differs from log(2z) by less than 4/10 of 1 percent, and increasingly less so, for arguments z whose magnitude exceeds 5. The advantageous numerical properties of arcsin h over logarithm are mathematical and inherent, and are not due to arcsin h's variance stabilization properties per se, e.g., of VST per se. The mathematical advantages are gained once VST is applied to gene expression signal data, and primarily employed for the benefits to subsequent statistical analysis accruing from stabilization of technical signal measurement variance.

Mathematical Refinement of the Basis for the Fundamental VST Equation t=f(s; c₁, c₂, c₃):

In Lin, et al., Nucleic Acids Research 36(2):ell, 1-9 (2008), the left-hand side of the legitimate generalized variance stabilizing integral applied to the accepted additive and multiplicative instrumental error model is written formally as an indefinite integral transform (i.e., with no explicit, nor implied in the paper, specific lower bound of integration). Formally so-written is not entirely correct. Rather, exactly the same integrand should appear in their eq. 6 but within a definite integral, i.e., an integral with an explicit lower-bound of integration appearing. The mathematical, and computer programmed implementation of VST employs the appropriate and correct definite integral version.

Visualization Globally of the Signal Distributional Effects of VST on Gene Expression Signal Data:

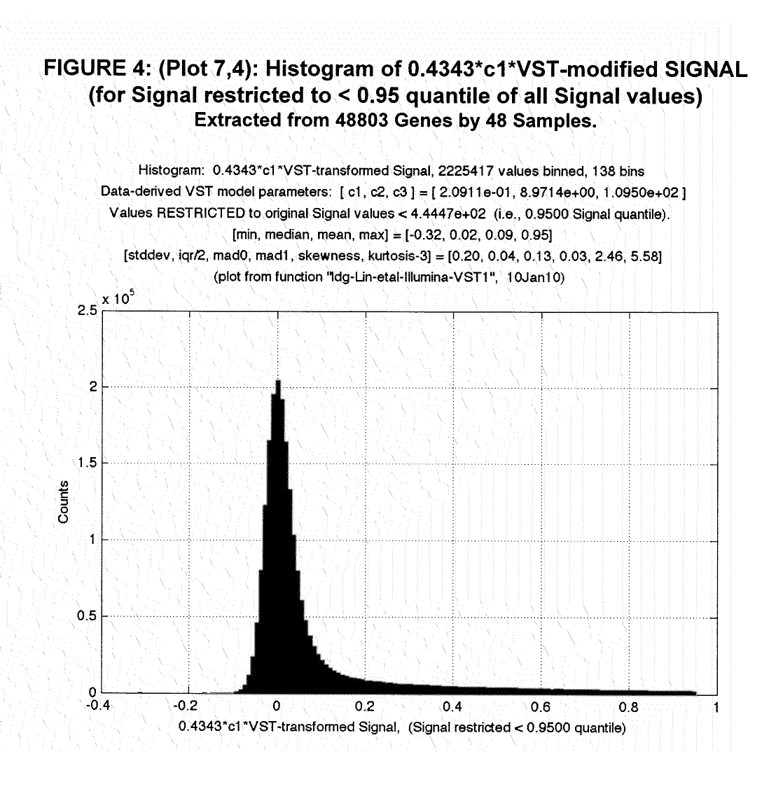

FIG. 4 (Plot 7,4) shows a histogram of all the signal values of the 48803 by 48 sample ensemble AF′T′ER the ensemble is “VST' ed”. There is no discernible humped feature suggesting a 2^{nd} Gaussian-like distribution being involved in the histogram of the transformed data (as is seen in the “pre-VST'ed” data in FIG. 1 (Plot 5,1). I.e., empirically the “VST'ed” signal data is much better behaved distributionally globally than before transformation by VST. FIG. 4 (Plot 7,4) shows also the right-wardly skewed tail capturing the relative minority of highly expressed, or “over-expressed”, i.e., very large, gene expression signals. The right-ward tail is also well behaved distributionally with respect to the entire empirical distribution. 7,3). For better viewing of the histogram along the horizontal axis, the signal data plotted in FIG. 4 has the largest 2% of VST largest signals omitted.

Table of Numerical Values, Sample by Sample, for VST Parameters c1, c2, c3:

Table 6 shows the typical study results for VST parameters c1, c2, and c2 when VST is applied not wholesale to the entire 48803 genes by 48 samples ensemble, as used in the exposition above. Rather, Table 6 shows the numerical results for the VST parameters, and other related quantities, and including descriptive statistics of the parameter (across 48 samples), when computed for each sample one by one from the study ensemble of 48 samples. When the signal values for a given sample (across all 48803 gene probes) from the study ensemble of 48 samples are transformed by the fundamental VST function (but multiplied by 1/ln(10) to put in log 10 units rather than in log base-e units), the triple of (c1, c2, c3) values are used in the fundamental VST equation for the given sample. The resulting “VST'ed” signal values (which are considered then to be in log 10 units) then are taken into the subsequent statistical analysis and outcome prediction analysis procedures.

TABLE 6
Data-derived VST c1, c2, c3 numerical values for 48 samples, each sample assessed
across 48803 gene probes.
Data-derived parameters c1, c2, c3 are obtained for each Sample by using
the function ldg_fast1_col_Illumina_VST1. For any given Sample (data column) . . .
VST model data-derived parameter values c1, c2, c3 are from linear fit
of sqrt(variance(positive signal) − c3) vs. c1 * signal + c2, for given Sample column, and
data-derived c3 is “average background noise” (averaged across genes). I.e.,
“average background noise” is mean variance of “not significant signal”
(i.e., averaged across all “not significant signal” genes among all 48803 genes).
For the VST model, “not significant signal” is signal for which Illumina Detection p-value
is greater than the SPECIFIED “not significant signal detection” threshold of 0.5.
“rms error” is the root mean squared difference between the VST model linear fit value
and the observed signal stddev, for which signal variance exceeds c3, for each of the 48
samples treated one by one by the VST procedure.

| | | ordinate | (avg. noise | data - | | | |
|---|---|---|---|---|---|---|---|
| | intercept | background) | linear fit | abscissa | (signal) | | |
| slope i | c1 | c2 | c3 | rms error | intercept | c1/sqrt(c3) | c2/sqrt(c3) |
| 1) | 0.1931 | 7.6704 | 73.5857 | 9.1133 | −39.7307 | 2.251e−02 | 8.942e−01 |
| 2) | 0.1909 | 7.6393 | 74.0352 | 9.0450 | −40.0199 | 2.218e−02 | 8.878e−01 |
| 3) | 0.1979 | 9.1056 | 122.3927 | 11.6411 | −46.0079 | 1.789e−02 | 8.231e−01 |
| 4) | 0.2009 | 9.5195 | 131.7722 | 11.6787 | −47.3758 | 1.750e−02 | 8.293e−01 |
| 5) | 0.2331 | 7.6188 | 119.7042 | 6.8953 | −32.6803 | 2.131e−02 | 6.964e−01 |
| 6) | 0.2362 | 9.8098 | 146.0437 | 17.6191 | −41.5246 | 1.955e−02 | 8.117e−01 |
| 7) | 0.2290 | 9.0556 | 90.0853 | 13.5222 | −39.5475 | 2.413e−02 | 9.541e−01 |
| 8) | 0.1945 | 8.4312 | 122.9463 | 5.3164 | −43.3471 | 1.754e−02 | 7.604e−01 |
| 9) | 0.2087 | 7.8182 | 82.8694 | 8.5509 | −37.4606 | 2.293e−02 | 8.588e−01 |
| 10) | 0.1978 | 11.0393 | 157.6520 | 11.3531 | −55.8165 | 1.575e−02 | 8.792e−01 |
| 11) | 0.1953 | 9.7066 | 135.9825 | 13.3452 | −49.6941 | 1.675e−02 | 8.324e−01 |
| 12) | 0.2040 | 6.5398 | 65.4384 | 5.7675 | −32.0535 | 2.522e−02 | 8.084e−01 |
| 13) | 0.1837 | 8.1626 | 78.9135 | 10.7090 | −44.4287 | 2.068e−02 | 9.189e−01 |
| 14) | 0.2310 | 9.2147 | 138.1069 | 11.5314 | −39.8952 | 1.965e−02 | 7.841e−01 |
| 15) | 0.2255 | 10.2855 | 151.4679 | 14.8532 | −45.6183 | 1.832e−02 | 8.357e−01 |
| 16) | 0.1987 | 10.6989 | 152.6931 | 13.5622 | −53.8530 | 1.608e−02 | 8.658e−01 |
| 17) | 0.2103 | 8.4860 | 82.9680 | 12.0448 | −40.3511 | 2.309e−02 | 9.316e−01 |
| 18) | 0.2280 | 8.2641 | 87.8796 | 10.6302 | −36.2386 | 2.433e−02 | 8.816e−01 |
| 19) | 0.1940 | 10.8655 | 149.2040 | 15.9561 | −56.0137 | 1.588e−02 | 8.895e−01 |
| 20) | 0.1803 | 8.8685 | 89.4246 | 11.6997 | −49.1945 | 1.906e−02 | 9.378e−01 |
| 21) | 0.2014 | 7.5389 | 75.6339 | 8.7054 | −37.4373 | 2.316e−02 | 8.669e−01 |
| 22) | 0.2113 | 8.0475 | 88.4602 | 7.6482 | −38.0828 | 2.247e−02 | 8.556e−01 |
| 23) | 0.2188 | 10.1163 | 142.0871 | 13.1008 | −46.2404 | 1.835e−02 | 8.487e−01 |
| 24) | 0.1924 | 8.4828 | 86.7673 | 10.9134 | −44.0834 | 2.066e−02 | 9.107e−01 |
| 25) | 0.2026 | 10.6932 | 158.1119 | 15.2558 | −52.7780 | 1.611e−02 | 8.504e−01 |
| 26) | 0.2236 | 8.9440 | 130.0491 | 10.9693 | −40.0067 | 1.960e−02 | 7.843e−01 |
| 27) | 0.2505 | 9.5965 | 143.2067 | 19.0065 | −38.3066 | 2.093e−02 | 8.019e−01 |
| 28) | 0.2411 | 8.8903 | 129.1467 | 19.0587 | −36.8714 | 2.122e−02 | 7.823e−01 |
| 29) | 0.1979 | 8.3725 | 86.1638 | 10.2309 | −42.3059 | 2.132e−02 | 9.020e−01 |
| 30) | 0.2176 | 10.1751 | 146.1900 | 16.2367 | −46.7593 | 1.800e−02 | 8.415e−01 |
| 31) | 0.2023 | 9.0555 | 90.7925 | 13.1548 | −44.7643 | 2.123e−02 | 9.504e−01 |
| 32) | 0.2119 | 6.5460 | 66.0484 | 7.1058 | −30.8866 | 2.608e−02 | 8.055e−01 |
| 33) | 0.2103 | 6.5344 | 67.5574 | 8.7749 | −31.0730 | 2.559e−02 | 7.950e−01 |
| 34) | 0.1882 | 10.3724 | 139.0566 | 15.0647 | −55.1031 | 1.596e−02 | 8.796e−01 |
| 35) | 0.2257 | 7.2784 | 78.3815 | 9.6685 | −32.2487 | 2.549e−02 | 8.221e−01 |
| 36) | 0.2071 | 8.2408 | 88.9377 | 10.2574 | −39.7922 | 2.196e−02 | 8.738e−01 |
| 37) | 0.2109 | 8.0180 | 79.2804 | 11.4981 | −38.0201 | 2.368e−02 | 9.005e−01 |
| 38) | 0.1918 | 8.4245 | 83.1117 | 11.0860 | −43.9300 | 2.104e−02 | 9.241e−01 |
| 39) | 0.2157 | 7.5638 | 90.2344 | 5.3902 | −35.0657 | 2.271e−02 | 7.963e−01 |
| 40) | 0.1922 | 10.4373 | 147.4283 | 14.9077 | −54.2927 | 1.583e−02 | 8.596e−01 |
| 41) | 0.2167 | 9.5285 | 139.8563 | 13.0149 | −43.9758 | 1.832e−02 | 8.057e−01 |
| 42) | 0.2221 | 8.0544 | 88.0398 | 10.7173 | −36.2644 | 2.367e−02 | 8.584e−01 |
| 43) | 0.1840 | 7.4687 | 74.5185 | 9.4471 | −40.5902 | 2.132e−02 | 8.652e−01 |
| 44) | 0.2284 | 6.4066 | 71.9591 | 6.3741 | −28.0441 | 2.693e−02 | 7.552e−01 |
| 45) | 0.2045 | 8.9891 | 126.7488 | 11.5178 | −43.9506 | 1.817e−02 | 7.984e−01 |
| 46) | 0.2041 | 9.7227 | 139.7254 | 12.1089 | −47.6280 | 1.727e−02 | 8.225e−01 |
| 47) | 0.2439 | 7.8124 | 118.4329 | 6.6700 | −32.0328 | 2.241e−02 | 7.179e−01 |
| 48) | 0.2302 | 7.5929 | 113.2130 | 7.8511 | −32.9839 | 2.164e−02 | 7.136e−01 |

Descriptive statistics of all the by quantities, column by column, i.e., descriptive
statistics computed across the 48 rows (i.e., samples) in each column:
ordinate (avg. noise data - (signal)

| | intercept | background) | linear fit | abscissa | | | |
|---|---|---|---|---|---|---|---|
| slope i | c1 | c2 | c3 | rms error | intercept | c1/sqrt(c3) | c2/sqrt(c3) |
| minimum: | 0.1803 | 6.4066 | 65.4384 | 5.3164 | −56.0137 | 1.575e−02 | 6.964e−01 |
| mean: | 0.2100 | 8.7022 | 109.2147 | 11.2619 | −41.7577 | 2.066e−02 | 8.431e−01 |
| median: | 0.2079 | 8.4844 | 102.0027 | 11.2196 | −40.4706 | 2.113e−02 | 8.495e−01 |
| maximum: | 0.2505 | 11.0393 | 158.1119 | 19.0587 | −28.0441 | 2.693e−02 | 9.541e−01 |
| stddev: | 0.0172 | 1.2283 | 30.5179 | 3.3775 | 7.1395 | 3.069e−03 | 6.061e−02 |

(where for all of the above, specified detection p-value threshold = 0.5)

### Example 3

This example includes a description of GVHD Class Divisions and statistical T-tests used for determining differences in patient GVHD outcome based upon HCT donor gene expression measurements.

A “class division” refers to direct numerical, mathematical, statistical, or computational comparisons between quantitative gene expression of donors whose respective transplanted patients have displayed one or more particular GVHD outcome Groups (e.g., class 1) vs. donors whose respective transplanted patients have displayed one or more other particular GVHD outcome Groups (e.g., class 2, and which is by definition of the 2-class comparison, different than class 1).

Class divisions involve comparisons between two classes and no comparisons among more than two classes at the same time. As can be seen in Table 7, a given well-defined class can by definition comprise more than one so-called “Group” of kinds of GVDH-related outcomes of corresponding transplanted patients. Thus, class divisions always involve exactly two defined classes; yet a given defined class can comprise more than one defined GVHD outcome Group.

TABLE 7
TTEST DIVISIONS

| T-test division | anyGVHD vs. noGVHD | cGVHD vs. noGVHD | aGVHD vs. noGVHD | a&cGVHD vs. noGVHD | a34GVHD vs. noGVHD |
|---|---|---|---|---|---|
| Class 1 | any GVHD | chronic GVHD | acute GVHD | acute and chronic GVHD | acute grade 3 or 4 GVHD |
| Class 1 GVHD | 2, 3, 4, 5, 6 | 2, 4, 6 | 3, 4, 5, 6 | 4, 6 | 5, 6 |
| Groups | | | | | |
| Class 1 sample total | n = 96 | n = 61 | n = 76 | n = 41 | n = 39 |
| Class 2 | no GVHD | no GVHD | no GVHD | no GVHD | no GVHD |
| Class 2 GVHD | 1 | 1 | 1 | 1 | 1 |
| Group | | | | | |
| Class 2 sample total | n = 26 | n = 26 | n = 26 | n = 26 | n = 26 |

In order to identify donor pre-transplant CD4+ T-cell RNA expression profiles predictive of HCT recipient GVHD outcome, conventional single-gene expression analysis was performed, i.e., single-variate, statistical T-tests (i.e., a one-dimensional form of LDA, linear discriminant analysis) (Sheskin, David J. Handbook of Parametric and Nonparametric Statistical Procedures, 3^{rd} Edition, Chapman & Hall/CRC Press, Boca Raton, Fla., 2004, esp. pp. 404-409.) for five 2-class divisions (Table 7), comparing samples from Group 1 (no acute and no chronic GVHD), to various combinations of GVHD-positive Groups.

Two types of T-tests for each of the 2-class divisions were carried out. One was a standard heteroscedastic, two-tailed T-test. The second was a measured gene expression signal “precision-weighted T-test” (also heteroscedastic, two-tailed) that takes the inherent numerical estimates of Illumina BeadArray measurement errors for each measured gene expression into account as reported in the so-called “bead standard error” variable provided by the Illumina platform (under contract with EA) in the standard IIlumina microarray measurement output file. The equations, and formulas used for the two T-tests are as follows:

Standard T-Test:

The 2-class two-tailed heteroscedastic T-test was carried out using class P (positive for GVHD outcome) and N (negative for GVHD outcome) probe signal value averages, ( P̅,̅ N̅), respective unequal variances, (s_{P}², s_{N}²), and respective sample totals per class, (n_{P}, n_{N}), according to the long-established standard statistical equations for the values of t and DF (degrees of freedom), and for which the p-values were determined computationally by invoking standard computer software (Excel or Matlab) T-test functions (i.e., equivalent to looking-up in standard T distribution tables).

$$\begin{matrix}t = \frac{\overline{P} - \overline{N}}{√ \frac{{s}_{P}^{2}}{{n}_{P}} + \frac{{s}_{N}^{2}}{{n}_{N}}} & ( 1 ) \\ D F = \frac{{( \frac{{s}_{P}^{2}}{{n}_{P}} + \frac{{s}_{N}^{2}}{{n}_{N}} )}^{2}}{\frac{{( \frac{{s}_{P}^{2}}{{n}_{P}} )}^{2}}{{n}_{P} - 1} + \frac{{( \frac{{s}_{N}^{2}}{{n}_{N}} )}^{2}}{{n}_{N .} - 1}} & ( 2 )\end{matrix}$$

Precision-Weighted T-Test:

The 2-class probe signal measurement precision-weighted T-test was carried using weighted averages, ( P̅_̅{̅w̅}̅, N̅_̅{̅w̅}̅, equations 13 and 14) and unequal compound variances (s_{uP}², s_{uN}², equations 20 and 21) for determining the values of t and DF, using the same fundamental statistical equations as for the standard, two tailed heteroscedastic T-test.

$$\begin{matrix}t = \frac{\overline{{P}_{w}} - \overline{{N}_{w}}}{\sqrt{\frac{{s}_{cP}^{2}}{{n}_{P}} + \frac{{s}_{cN}^{2}}{{n}_{N}}}} & ( 3 ) \\ D F = \begin{matrix}{( \frac{{s}_{cP}^{2}}{{n}_{P}} + \frac{{s}_{cN}^{2}}{{n}_{N}} )}^{2} \\ \frac{{( \frac{{s}_{cP}^{2}}{{n}_{P}} )}^{2}}{{n}_{P} - 1} + \frac{{( \frac{{s}_{cN}^{2}}{{n}_{N}} )}^{2}}{{n}_{N .} - 1}\end{matrix} & ( 4 )\end{matrix}$$

The weights used in the precision-weighted T-test were determined as described below, based on the reciprocals of the Bead Standard Error (be) provided for each sample from VST processing of the Illumina data. Note: Computed weights as employed average to 1 and sum for each class to the total sample number per class, i.e. n_{P}, N_{N}. This assures that, if all the weights for a class are the same, the weighted expression values and their average will not change from the respective non-weighted values within each class.

Definition of pre-weight for the i-th sample in each class (pw_{Pi}, pw_{Ni}).

$$\begin{matrix}{pw}_{Pi} = \frac{1}{{be}_{Pi}} & ( 5 ) \\ {pw}_{Ni} = \frac{1}{{be}_{Ni}} & ( 6 )\end{matrix}$$

Definition of average pre-weight ( p̅w̅_̅{̅P̅}̅, p̅w̅_̅{̅N̅}̅) for each class.

$$\begin{matrix}\overline{{pw}_{P}} = \frac{{\sum }_{i}^{{n}_{P}} b {e}_{Pi}}{{n}_{P}} & ( 7 ) \\ \overline{{pw}_{N}} = \frac{{\sum }_{i}^{{n}_{N}} {be}_{Ni}}{{n}_{N}} & ( 8 )\end{matrix}$$

Definition of the weight (w_{Pi}, w_{Ni}) for the i-th sample in each class.

$$\begin{matrix}{w}_{Pi} = \frac{{pw}_{Pi}}{\overline{{pw}_{P}}} & ( 9 ) \\ {w}_{Ni} = \frac{{pw}_{Ni}}{\overline{{pw}_{N}}} & ( 10 )\end{matrix}$$

Determination of Weighted Individual (P_{wPi}, N_{wNi}), and Class Averages ( P̅_̅{̅w̅}̅, N̅_̅{̅w̅}̅) Probe Signal Values.

Note: This is based on the simple concept of multiplying each sample value (P_{Pi}, N_{Ni}), the sample weight, and then averaging the weighted values for each class.

$$\begin{matrix}{P}_{wPi} = {w}_{pi} \cdot {P}_{Pi} & ( 11 ) \\ {N}_{wNi} = {w}_{Ni} \cdot {N}_{Ni} & ( 12 ) \\ \overline{Pw} = \frac{{\sum }_{i}^{{n}_{P}} {P}_{wPi}}{{n}_{P}} & ( 13 ) \\ \overline{{N}_{w}} = \frac{{\sum }_{i}^{{n}_{N}} {N}_{wNi}}{{n}_{N}} & ( 14 )\end{matrix}$$

Determination of Weighted Class Variances (s_{wP}², s_{wN}²).

Note: The concept is that (1) a difference is formed, squared and weighted (dev_{wPi}², dev_{wNi}²) between the measured signal and weighted average (reflecting the variance contribution of each sample), and (2) this weighted, squared deviation (variance contribution) is then averaged to generate a total weighted variance (s_{wP}², s_{wN}²).

$$\begin{matrix}{dev}_{wNi}^{2} = {w}_{Ni} - {[ {( N ]}_{Ni} - \overline{{N}_{w}} )}^{2} & ( 15 ) \\ {s}_{wP}^{2} = \frac{{\sum }_{i}^{{n}_{P}} {dev}_{wPi}^{2}}{{n}_{P} - 1} & ( 16 ) \\ {s}_{wN}^{2} = \frac{{\sum }_{i}^{{n}_{N}} {dev}_{wNi}^{2}}{{n}_{N} - 1} & ( 17 )\end{matrix}$$

Determination of Bead Variance Contribution to Variance within a Class (s_{beP}², s_{beN}²).

Note: This is important to reflect overall differences in bead standard errors between the classes. Since for the weighted class variance (s_{wP}², s_{wN}²), all of the weighting so far is restricted within each class, it does not reflect any, or major, differences in bead standard errors per se between the classes, that importantly can contribute to the quantitation of confidence in the separation of the classes.

$$\begin{matrix}{s}_{beP}^{2} = \frac{{\sum }_{i}^{{n}_{P}} {be}_{Pi}^{2}}{{n}_{P} - 1} & ( 18 ) \\ {s}_{beN}^{2} - \frac{{\sum }_{i}^{{n}_{N}} {be}_{Ni}^{2}}{{n}_{N} - 1} & ( 19 )\end{matrix}$$

Determination of Compound Variance as Sum of Weighted Signal Variance and Bead Variance Contributions to Class Variance.

By adding these two class-wise variances (s_{be}² and s_{w}²), the confidence in individual sample measurements relative to their measurement error (be) is taken into account (through s_{be}²), as well as the average measurement error of each class (as within-class weighted variance s_{w}²). Therefore, more confidence, resulting in numerically lower (i.e., more statistically significant) and more trust-worthy p-values, will be placed in sample measurements and classes having smaller measurement errors (be).

s_{cP}²=s_{wP}²+s_{beP}² (20)

s_{cN}²=s_{wN}²+s_{beN}² (21)

### Example 4

This example includes a description of the gene expression analysis and GVHD outcome-prediction of donor HCT.

Overview and Details on “Class Divisions”, Different Possible Patient GVHD Outcomes, and Tallies of True and False Positive and Negative Computational\Statistical Outcome-Predictive Classification Groups:

For each class division and probe LDA was carried out (conventional linear discriminant analysis, the associated p-value being equivalent to a T-test when single-variate; Richard O. Duda, Peter E. Hart, & David G. Stork, Pattern Classification, 2^{nd} Ed., John Wiley & Sons, NY, 2001) to obtain predicted GVHD outcome classification accuracies. LDA is used to classify each sample (i.e., donor) as GVHD positive (i.e. induces GVHD in the recipient), or GVHD negative (i.e. does not induce GVHD in the recipient), depending on whether the RNA expression value is above, or below, a threshold (in this particular study, the threshold for LDA was exactly half-way between the averages of the positive and negative GVHD sample RNA expression values from the respective two classes involved). Depending on whether samples are classified computationally\statistically correctly or not, they fall into one of four different categories, or groups (“group” with lower-case “g”, not to be confused with GVHD-related “Group” with upper-case “g”):

1. TN (True Negative), actual GVHD negative sample classified as negative by computation
2. FN (False Negative), actual GVHD positive sample classified as negative by computation
3. FP (False Positive), actual GVHD negative sample classified as positive by computation
4. TP (True Positive), actual GVHD positive sample classified as positive by computation

The same nomenclature, TN, FN, FP, TP was used to define a classification situation as above, as well as representing either the numbers (i.e., counts) of so classified donors, the usages of which should be clear from the context. The term “sample” is used interchangeably for “donor” or for analyzed quantitative gene expression of a donor in describing the data. Total sample counts are then summed for each group (group with small “g”):

1. TNtot=total TN samples
2. FNtot=total FN samples
3. FPtot=total FP samples
4. TPtot=total TP samples

The total GVHD negative, Ntot, and GVHD positive, Ptot, samples contributing to the study are sums of occurrences, defined as follows:

### 1. Ntot=TNtot+FPtot

### 2. Ptot=TPtot+FNtot

Note: An established convention in statistics, classification statistics, and datamining fields is that when “binary” outcome categories are being considered, i.e., “true” or “false”, then a “False Positive” event is counted as a “negative” event because it is not “positive”; hence, the definition of Ntot seen above where FPtot is added to TNtot. Analogously, for “False Negative” events and the definition of Ptot in line 2 immediately above.

### Example 5

This example includes a description of an exemplary Gene Expression Voting Model, RNA20.

The RNA20 Voting Scheme of LDA Models:

For the exemplary RNA20 model (anyGVHD vs. noGVHD division), each of the component RNA marker LDA models provides a yes\no (1\0) prediction, i.e. vote, for the GVHD negative outcome for each sample (each of 20 RNA species' series of GVHD negative votes over the 122 HCT donors is displayed in a separate row in Table 8). All GVHD negative votes across the 20 RNA species are counted for each sample, and divided by the total number of RNA species, i.e., 20, to arrive at the “GVHD negative score”, displayed below the individual marker voting profiles (Table 8).

A sample is finally classified by this 20 marker model as GVHD negative if the GHVD negative score is above a (user selected) threshold of 0.77, i.e., at least a 77% majority of the total 20 RNA species-based votes is required for a sample to be classified as “GVHD negative” (values in white text and black or dark grey background). Correspondingly, a sample is classified as “GVHD positive” if the GHVD negative score is below a threshold of 0.77 (values in black text and white or light grey background). The GVHD negative score, the total numbers of True Negatives, False Positives and Ntot (Total row), reported for Group 1, and the total numbers of False Negatives, True Positives, and Ptot (Total row), reported for each of the Groups 2 through 6 are shown in Table 8.

TABLE 8
RNA VOTING SCHEME

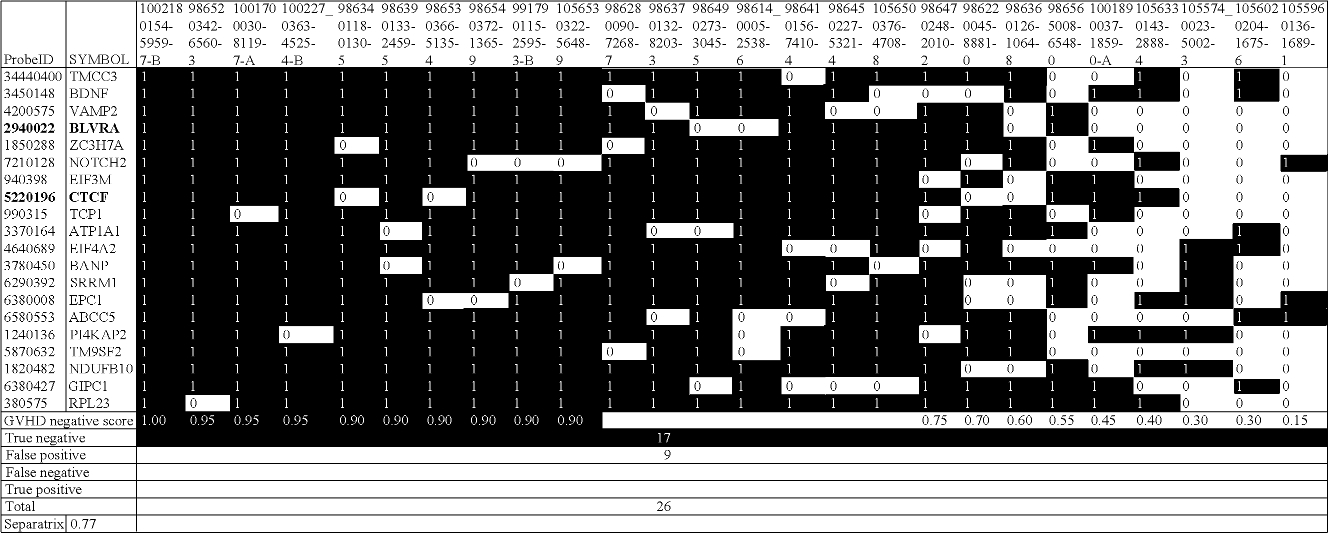

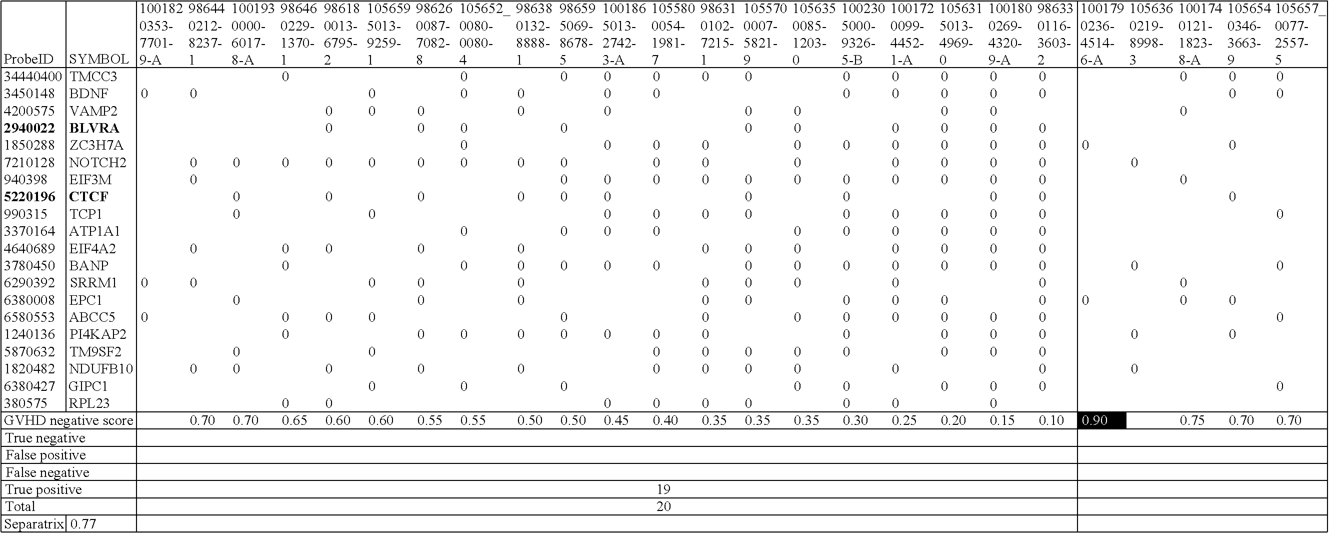

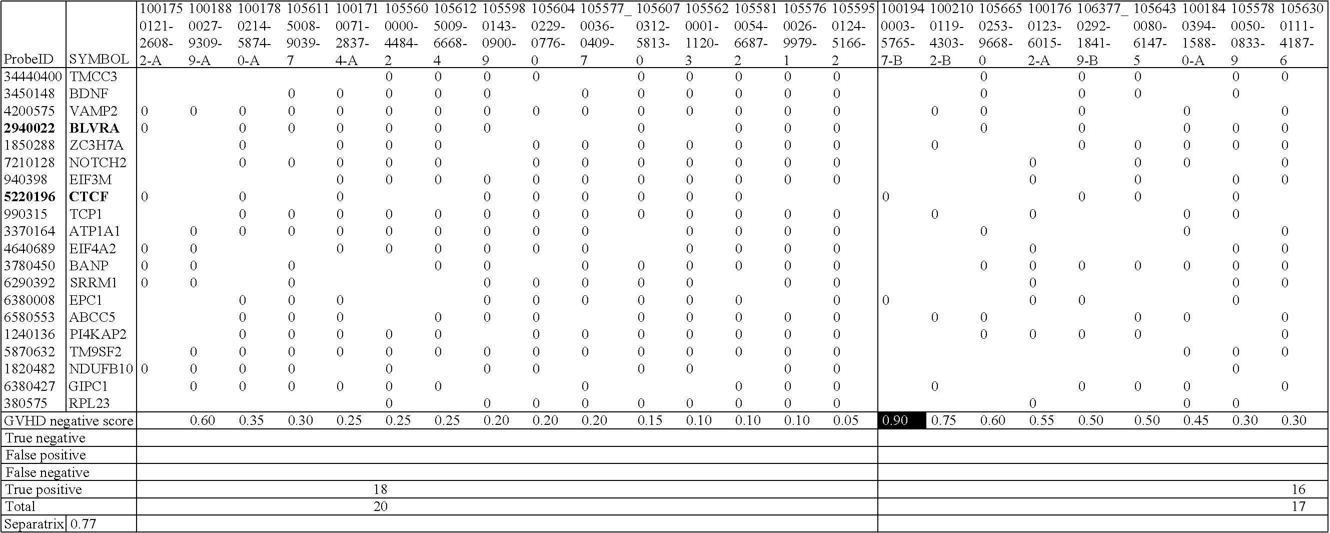

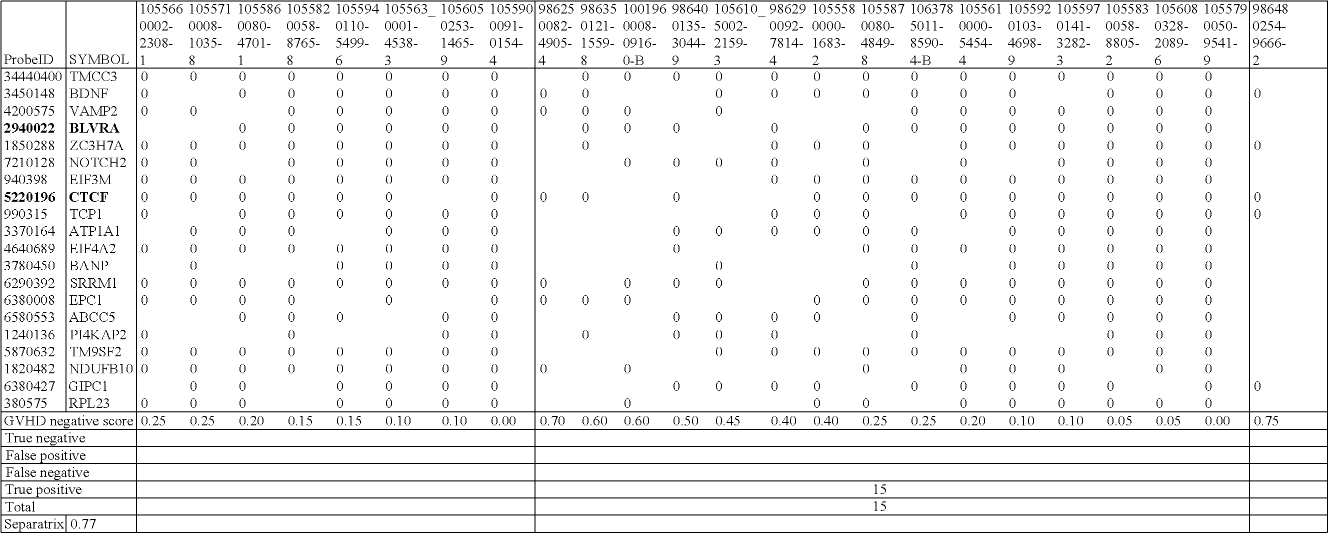

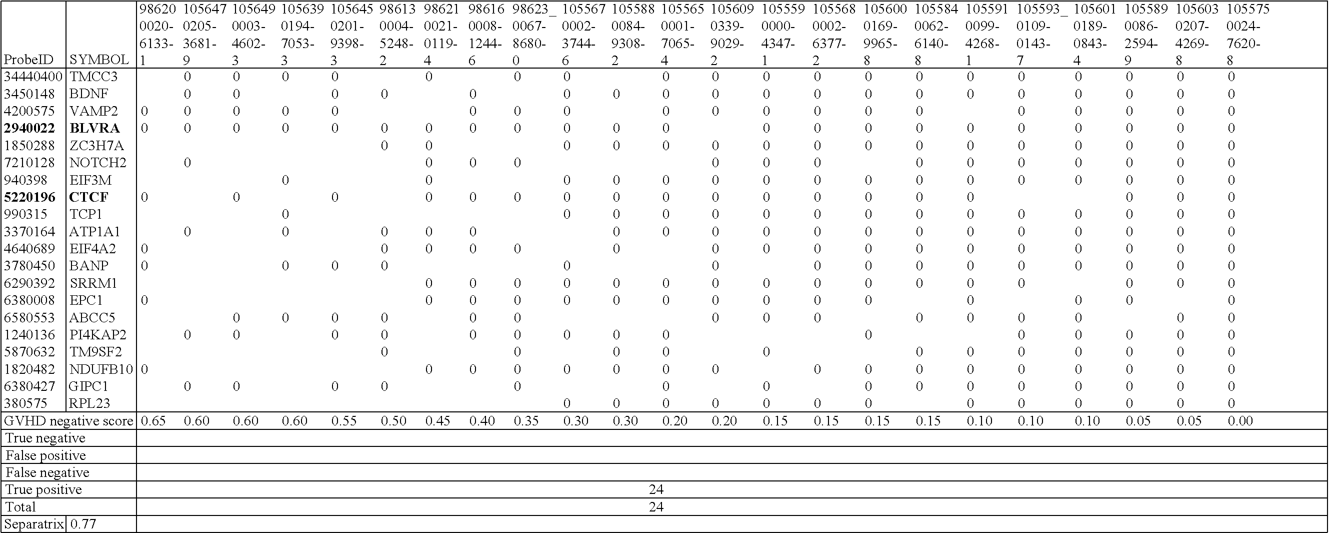

Details on the 20 contributing RNA species and their individual LDA classification performance are listed in Table 9.

TABLE 9
RNA20 LIST

| | | | | | | | | bal | bal | bal |
|---|---|---|---|---|---|---|---|---|---|---|
| | | | | | | | | accuracy | specificity, | negative |
| | | | | p | | | | (TP + TN)/ | true | predictive |
| | | | | precision- | p hetero- | | | (TP + | negative | value |
| | | | Detection | weighted | scedastic | larger | max | FP + | rate TN/ | TN/(TN + |
| ProbeID | Gene Name | SYMBOL | Rank | T-test | T-test | average | average | TN + FN) | (TN + FP) | FN) |
| 3440400 | transmembrane and coiled- | TMCC3 | 21640 | 0.022308 | 0.000005 | N | 0.62 | 0.77 | 0.81 | 0.75 |
| | coil domain family 3 | | | | | | | | | |
| 3450148 | brain-derived neurotrophic | BDNF | 21640 | 0.040604 | 0.000936 | N | 0.67 | 0.72 | 0.73 | 0.71 |
| | factor, transcript variant 6 | | | | | | | | | |
| 4200575 | vesicle-associated membrane | VAMP2 | 1070 | 0.005441 | 0.002226 | P | 2.50 | 0.68 | 0.65 | 0.68 |
| | protein 2 | | | | | | | | | |
| 2940022 | biliverdin reductase A | BLVRA | 4090 | 0.010430 | 0.004310 | N | 1.67 | 0.69 | 0.69 | 0.69 |
| 1850288 | zinc finger CCCH-type | ZC3H7A | 10651 | 0.027973 | 0.000412 | N | 0.88 | 0.70 | 0.73 | 0.69 |
| | containing 7A | | | | | | | | | |
| 7210128 | Notch homolog 2 | NOTCH2 | 6480 | 0.040708 | 0.009368 | N | 1.22 | 0.68 | 0.69 | 0.68 |
| 940398 | eukaryotic translation | EIF3M | 2425 | 0.018840 | 0.004648 | N | 1.93 | 0.71 | 0.77 | 0.68 |
| | initiation factor 3, | | | | | | | | | |
| | subunit M | | | | | | | | | |
| 5220196 | CCCTC-binding factor | CTCF | 6480 | 0.030919 | 0.006569 | P | 1.22 | 0.69 | 0.73 | 0.67 |
| 990315 | t-complex 1, transcript | TCP1 | 3192 | 0.002837 | 0.000206 | N | 1.87 | 0.68 | 0.73 | 0.67 |
| | variant 1 | | | | | | | | | |
| 3370164 | ATPase, Na+/K+ | ATP1A1 | 2425 | 0.017986 | 0.001829 | P | 2.05 | 0.68 | 0.73 | 0.67 |
| | transporting, alpha | | | | | | | | | |
| | 1 polypeptide, | | | | | | | | | |
| | transcript variant 1 | | | | | | | | | |
| 4640689 | eukaryotic translation | EIF4A2 | 227 | 0.005331 | 0.001070 | P | 3.69 | 0.66 | 0.69 | 0.66 |
| | initiation factor 4A, | | | | | | | | | |
| | isoform 2 | | | | | | | | | |
| 3780450 | BTG3 associated nuclear | BANP | 6480 | 0.028352 | 0.017499 | P | 1.14 | 0.70 | 0.77 | 0.67 |
| | protein, transcript | | | | | | | | | |
| | variant 2 | | | | | | | | | |
| 6290392 | serine/arginine repetitive | SRRM1 | 815 | 0.014103 | 0.001962 | P | 2.70 | 0.66 | 0.69 | 0.65 |
| | matrix 1 | | | | | | | | | |
| 6380008 | enhancer of polycomb | EPC1 | 4090 | 0.020545 | 0.000762 | P | 1.56 | 0.69 | 0.77 | 0.67 |
| | homolog 1 | | | | | | | | | |
| 6580553 | ATP-binding cassette, | ABCC5 | 8164 | 0.033021 | 0.000792 | P | 0.87 | 0.67 | 0.73 | 0.65 |
| | sub-family C, member | | | | | | | | | |
| | 5, transcript variant 1 | | | | | | | | | |
| 1240136 | phosphatidylinositol 4- | PI4KAP2 | 2425 | 0.012117 | 0.000972 | P | 1.91 | 0.69 | 0.77 | 0.66 |
| | kinase, catalytic, | | | | | | | | | |
| | alpha polypeptide | | | | | | | | | |
| | pseudogene 2 | | | | | | | | | |
| 5870632 | transmembrane 9 | TM9SF2 | 2425 | 0.007116 | 0.003365 | N | 2.03 | 0.65 | 0.69 | 0.64 |
| | superfamily member 2 | | | | | | | | | |
| 1820482 | NADH dehydrogenase | NDUFB10 | 1849 | 0.005686 | 0.004427 | N | 2.23 | 0.70 | 0.81 | 0.67 |
| | 1 beta subcomplex, | | | | | | | | | |
| | 10, 22 kDa | | | | | | | | | |
| 6380427 | GIPC PDZ domain | GIPC1 | 4090 | 0.019668 | 0.008262 | P | 1.43 | 0.66 | 0.73 | 0.64 |
| | containing family, | | | | | | | | | |
| | member 1, transcript | | | | | | | | | |
| | variant 3 | | | | | | | | | |
| 380575 | ribosomal protein L23 | RPL23 | 489 | 0.011463 | 0.001349 | N | 3.16 | 0.68 | 0.85 | 0.64 |

Balancing effects due to inherent differences of numbers of donors involved in representing different classifications with respect to True and False Positives and Negatives: In an effort to equally balance numerically the contributions from the GVHD positive and negative sample groups, the relative contributions of all 4 outcome classification groups are determined, balancing for inherent inequalities in the total GVHD positive and negative groups' sizes, i.e., numbers of respective samples involved:

### 1. TNbal=0.5 (TNtot/Ntot)

### 2. FNbal=0.5 (FNtot/Ptot)

### 3. FPbal=0.5 (FPtot/Ntot)

### 4. TPbal=0.5 (TPtot/Ptot)

The balanced GVHD positive and negative sample contributions now each equal to 0.5, and sum to 1:

### 1. Nbal=TNbal+FPbal=0.5

### 2. Pbal=TPbal+FNbal=0.5

### 3. Pbal+Nbal=1

Using the 4 balanced outcome classification groups, 5 different balanced outcome prediction performance measurements are determined (from here on below, all usage of the terms TN, FN, FP and TP refer to TNbal, FNbal, FPbal and TPbal, respectively):

### 1. Balanced NPV (Negative Predictive Value)=TN/(TN+FN)

Fraction of samples that were classified as negative which are truly negative.
   2. Balanced TNR (True Negative Rate) or specificity=TN/(TN+FP)
Fraction of total negative samples that were correctly classified.

### 3. Balanced PPV (Positive Predictive Value)=TP/(TP+FP)

Fraction of samples that were classified as positive which are truly positive.
   4. Balanced TPR (True Positive Rate) or sensitivity=TP/(TP+FN)
Fraction of positive samples that were correctly classified.

### 5. Balanced Accuracy=(TP+TN)/(TP+FP+TN+FN)

Fraction of total samples that were correctly classified.

Note: The fundamental definitions of NPV, TNR, PPV, and TPR are standard conventional definitions in statistics, classification statistics, and datamining. The balanced versions hence rely on the standard versions; however, they employ the analogous balanced versions of TN, FN, FP, and TP. From here on below, unless otherwise stated, all usage of the terms NPV, TNR, PPV, TPR and Accuracy refer to Balanced NPV, Balanced TNR, Balanced PPV, Balanced TPR and Balanced Accuracy.

### Example 6

This example includes a description of gene expression analysis results and prediction of GVHD outcomes, based upon an exemplary RNA20 model.

All 122 HCTs in the study correspond to HLA 10/10 matched unrelated donor transplantations, reflecting the majority of annual transplantations in the U.S. As discussed, transplant GVHD outcomes were categorized into six different Groups (Table 5).

Groups are numbered in order of increasing GVHD severity, beginning with Group 1 exhibiting neither acute nor chronic GVHD, and ending with Group 6, showing severe acute grade 3 or 4 GVHD and extensive chronic GVHD. Group 5 also shows grade 3 or 4 GVHD, but no chronic GVHD. Group 4 and Group 3 show grade 1 or 2 acute GVHD, with and without chronic GVHD, respectively. Group 2 shows only chronic GVHD and no acute GVHD. Acute grade 3 or 4 GVHD characterize the most intense and life-threatening form of GVHD, while acute grade 1 or 2 GVHD is much less severe and occasionally may be considered mild. The grade classifications of acute GVHD are multi-symptom diagnostic gradations well-established in medical \oncologic practice for physicians' gradings of GVHD severity, and analogously so for extensive, or not extensive, chronic GVHD. Although the definitions of the Groups are per se, they are medically meaningful GVHD-severity groups established by the experts of the NMDP.

Gross expression level trends among HCT donors associated with different GVHD Groups:

To characterize the relationship between donor CD4+ T-cell RNA expression profile and HCT recipient GVHD outcomes, and to distinguish potential biases in the dataset from biologically rooted relationships, the overall behavioral trends of GVHD Group average RNA expression levels as rank orderings over the microarray gene expression probes was analyzed (Table 10). The GVHD Group RNA expression rank order is determined for each gene probe as the rank of the average gene expression level for each of the six Groups in ascending order (i.e., higher levels of expression results in higher rank). The dataset then is separated into two subsets, denoted “N>PSubset” and “P>NSubset”, according to whether the average RNA expression of the GVHD negative samples (Group 1) is higher, or lower, than for the GVHD positive samples (Groups 2 though 6), respectively. For each of these two subsets, the median GVHD Group RNA expression rank, i.e. the “Rank”, is determined within two differently defined sets of probes, i.e., (1) comprising all 48,803 probes, i.e. the “N>P Total Subset” and “P>N Total Subset”, and (2) comprising a select subset of 1024 probes having T-test p-values<=0.05 for both heteroscedastic T-tests and precision-weighted T-tests as previously described. Such is carried out for the anyGVHD vs. noGVHD class division (Table 6), i.e. the “N>P Select Subset” and “P>N Select Subset”.

TABLE 10
GROUP ORDER

| | | GVHD negative > | | | | | | |
|---|---|---|---|---|---|---|---|---|
| | | GVHD positive | | | | | | |
| | | average expression | | | | | | |
| | | N > P Rank | N > P Rank | N > P Rank | N > P Rank | N > P Rank | N > P Rank | Pearson R |
| | | G1: | G2: | G3: | G4: | G5: | G6: | R: |
| | | a-no&c- | a-no&ex- | a-12&no- | a-12&ex- | a-34&no- | a-34&ex- | Rank series, |
| | | noGVHD | cGVHD | cGVHD | cGVHD | cGVHD | cGVHD | G1-6 |
| | | (26) | (20) | (20) | (17) | (15) | (24) | order |
| Total 48803 probes | median | 5.0 | 3.0 | 3.0 | 3.0 | 3.0 | 3.0 | −0.65 |
| precision-weighted AND | median | 6.0 | 4.0 | 4.0 | 3.0 | 2.0 | 2.0 | −0.95 |
| heteroscedastic T-test | | | | | | | | |
| p-value cutoff <=0.05 | | | | | | | | |

| | | GVHD positive > | | | | | | | | | | |
|---|---|---|---|---|---|---|---|---|---|---|---|---|
| | | GVHD negative | | | | | | | | | | |
| | | average expression | | | | | | | | | | |
| | | P > N | P > N | P > N | P > N | P > N | P > N | | | | | |
| | | Rank | Rank | Rank | Rank | Rank | Rank | Pearson R | | | | |
| | | G1: | G2: | G3: | G4: | G5: | G6: | R: Rank | | | | |
| | | a-no&c- | a-no&ex- | a-12&no- | a-12&ex- | a-34&no- | a-34&ex- | series, | | | | |
| | | noGVHD | cGVHD | cGVHD | cGVHD | cGVHD | cGVHD | G1-6 | | | | |
| | | (26) | (20) | (20) | (17) | (15) | (24) | order | | N > P | P > N | total |
| Total 48803 probes | median | 2.0 | 4.0 | 4.0 | 4.0 | 4.0 | 4.0 | 0.65 | total | 21790 | 27013 | 48803 |
| precision-weighted | median | 1.0 | 2.0 | 4.0 | 4.0 | 5.0 | 4.0 | 0.85 | total | 197 | 118 | 315 |
| AND hetero- | | | | | | | | | | | | |
| scedastic T-test | | | | | | | | | | | | |
| p-value cutoff <= | | | | | | | | | | | | |
| 0.05 | | | | | | | | | | | | |

For the N>P Total and Select Subsets, the Rank (median RNA expression rank) for Group 1, compared to each of the Group 2 to Group 6 Ranks, is consistently greater for the N>P Subset, without exception. Likewise, for the P>N Total and Select Subsets, the Rank for Group 1, compared to each of the Group 2 to Group 6 Ranks, is consistently smaller for the P>N Subset, without exception. (Table 10). Note that the selection criteria for the N>P and P>N subsets are restricted to comparing the average of Group 1 to the average of the combined samples of Groups 2 to 6, and not the individual averages for Groups 2 to 6. Therefore, (1) the applied selection criteria for the N>P and P>N Subsets do not necessarily guarantee that the Ranks of Groups 2 to 6 need uniformly to be greater or smaller than the Group 1 Rank, and (2) the fact that they actually are, demonstrates that there is no strong bias within any one of the Group 2 to 6 members that would place its Rank on the other side of the Group 1 Rank compared to the other Group 2 to Group 6 Ranks.

Furthermore, for the N>P Total Subset and P>N Total Subset, the Ranks for Groups 2 to 6 are all the same, demonstrating a high-level of uniformity of the Groups 2 to 6 Ranks over all the surveyed 48,803 microarray probes (Table 10). In contrast, for the select 1,024 GVHD outcome associated probes, the Rank order within Groups 2 to 6 shows a clear descending trend from Rank 4 to 2 for the N>P Select Subset, and ascending trend from Rank 2 to 5 within the P>N Select Subset, in parallel to increasing GVHD Group number and associated severity of GVHD. This deviation from the Group 2 to 6 Rank uniformity (observed above for the total set of 48,803 probes) is not indicative of an arbitrary bias. Rather, it signifies an ordered, parallel trend, where magnitude of gene expression correlates with severity of GVHD, measurably evidenced in very high magnitude Pearson correlations (R) of −0.95 for the N>P Select Subset, and +0.92 for the P>N Select Subset, between Rank order and GVHD Group number.

To summarize, the key insight from the strong correlation of GVHD Group disease severity order with Rank (median RNA expression rank) order for the 1,024 probes of the N>P Select Subset and P>N Select Subset (Table 10), is that the selection of these probes according to T-test performance for the anyGVHD vs. noGVHD class division, did not per se select for any orderings or distinguishing features within the 5 GVHD positive Groups 2 to 6. This is because the samples of Groups 2 to 6 were simply pooled for the T-test analysis, thereby losing all information on specific GVHD positive Group sources. Therefore, the observed Rank order within the GVHD positive Groups is an inherent, natural biological property exhibited by this select set of RNA profiles, independent of the means by which the probes were selected in the statistical analysis. In other words, the strong Pearson correlations of the Group 2 to 6 Ranks with the GVHD Group numbers could not have been inadvertently imposed by the analysis and processing of the data as statistical artifact, but indeed reflects the workings of specific molecular profiles underlying the Ranks and their association with actual biologically manifested GVHD intensity.

GVHD Outcome Prediction Revealed No Transplant Center-Associated Biases:

Below as concrete examples, the GVHD-outcome predictive behavior and sample transplant center source distributions for two specific individual RNA expression predictors were examined. In particular, “CTCF” (CCCTC-binding factor), for which expression levels tend to increase with GVHD intensity; “BLVRA” (biliverdin reductase A), for which expression levels tend to decrease with GVHD intensity; and “RNA20” (component RNA species listed in Table 9), an exemplary 20 RNA expression set “voting” model (METHODS and Table 8).

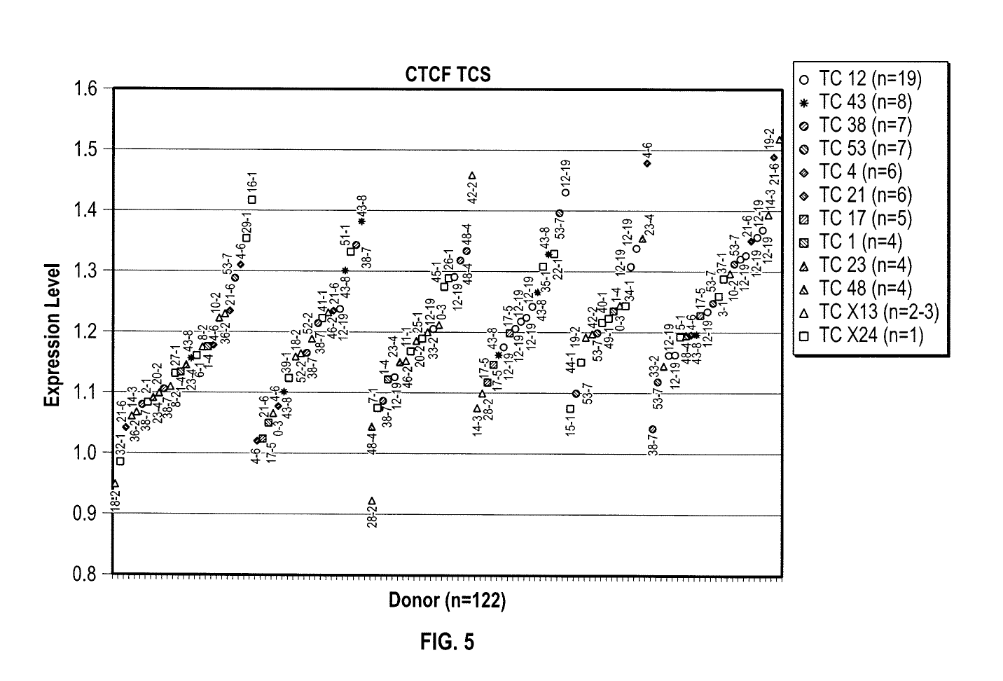

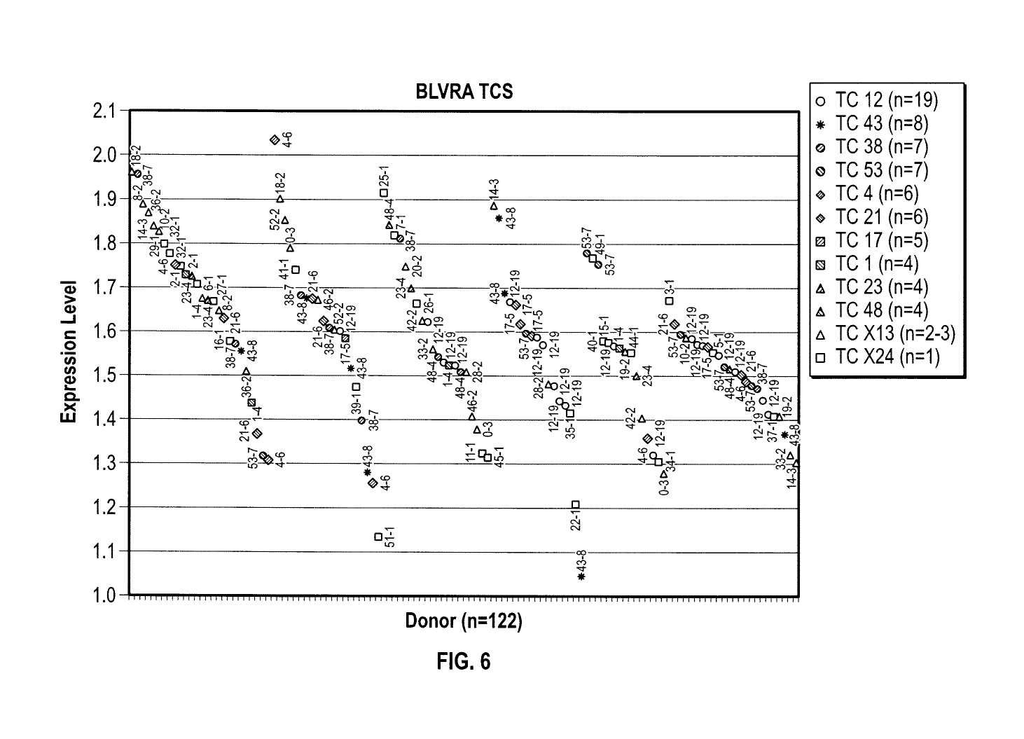

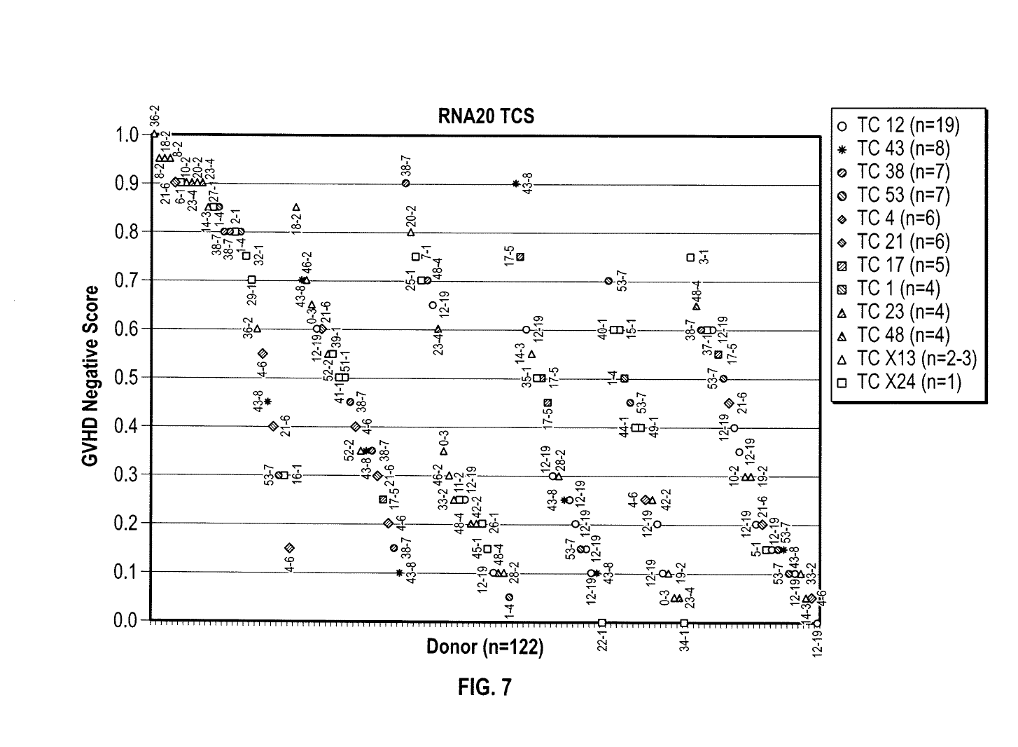

RNA expression measurement values are plotted for all 122 samples in ascending order for each of the six GVHD outcome classes, and labeled according to the samples' transplant center sources (TCS) (FIGS. 5-7, CTCF TCS, BLVRA TCS, RNA20 TCS). Transplant centers providing at least 4 samples are labeled with separate colors (n=85), centers providing 2-3 samples are labeled by triangles (n=13), and centers providing only one sample each are labeled by squares (n=24). Each data point is also labeled with the number of the transplant center (actual names of the centers were thus far blinded), followed by the numbers of samples provided by that center after the dash. Note in all three examples (CTCF, BLVRA and the RNA20 model), samples from multi-center and single center sources appear to be evenly distributed, and show no clustering within specific expression value ranges, or association biases toward specific GVHD outcome groups. In spite of any potential variations introduced by the different transplant centers that might distort or bias the gene expression assays or GVHD outcome attribution, each of these three concrete examples shows strong GVHD outcome prediction capability in terms of T-test performance and LDA accuracy measures (Table 11).

TABLE 11
PERFORMANCE

| | RNA marker or set | | | | | | |
|---|---|---|---|---|---|---|---|
| | CTCF | BLVRA | Select 20 | Select 20 | Select 20 | Select 20 | Select 20 |
| | GVHD outcome class division | | | | | | |
| | anyGVHD | anyGVHD | anyGVHD | cGVHD vs. | aGVHD vs. | a&cGVHD | a34GVHD |
| | vs. noGVHD | vs. noGVHD | vs. noGVHD | noGVHD | noGVHD | vs. noGVHD | vs. noGVHD |
| Post-test GVHD risk percentage FN/(TN + FN) | 33% | 31% | 6% | 5% | 6% | 4% | 0% |
| Post-test GVHD risk reduction Ptot − | 17% | 19% | 44% | 45% | 44% | 46% | 50% |
| FN/(TN + FN) | | | | | | | |
| Post-test relative GVHD risk reduction | 35% | 38% | 88% | 90% | 89% | 93% | 100% |
| [Ptot − FN/(TN + FN)]/Ptot | | | | | | | |
| Negative Predictive Value TN/(TN + FN) | 0.67 | 0.69 | 0.94 | 0.95 | 0.94 | 0.96 | 1.00 |
| True Negative Rate (specificity) TN/(TN + FP) | 0.73 | 0.69 | 0.65 | 0.65 | 0.65 | 0.65 | 0.65 |
| | | | | | | 0.31 | |
| Positive Predictive Value TP/(TP + FP) | 0.71 | 0.69 | 0.73 | 0.74 | 0.74 | 0.74 | 0.74 |
| True Positive Rate (sensitivity) TP/(TP + FN) | 0.65 | 0.69 | 0.96 | 0.97 | 0.96 | 0.98 | 1.00 |
| Accuracy (TP + TN)/(TN + FN + FP + TP) | 0.69 | 0.69 | 0.81 | 0.81 | 0.81 | 0.81 | 0.83 |
| p heteroscedastic T-test | 6.57E−03 | 4.31E−03 | 1.10E−08 | 2.53E−08 | 2.93E−09 | 5.81E−09 | 1.66E−09 |

Outcome Prediction Observations with Respect to Different RNA Models CTCF, BLVRA and RNA20:

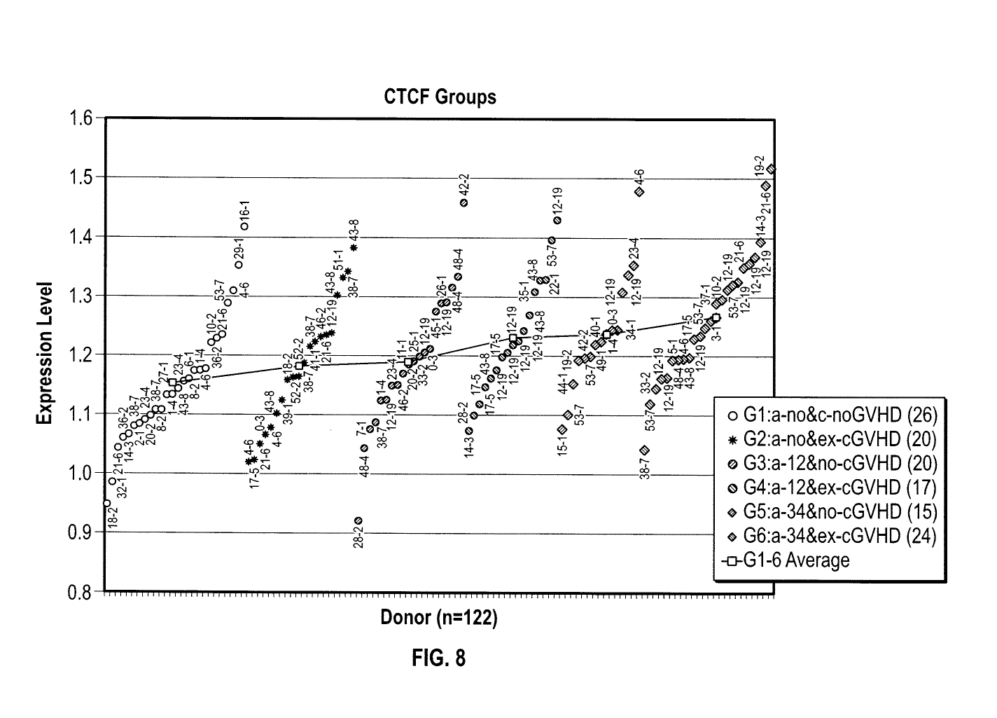

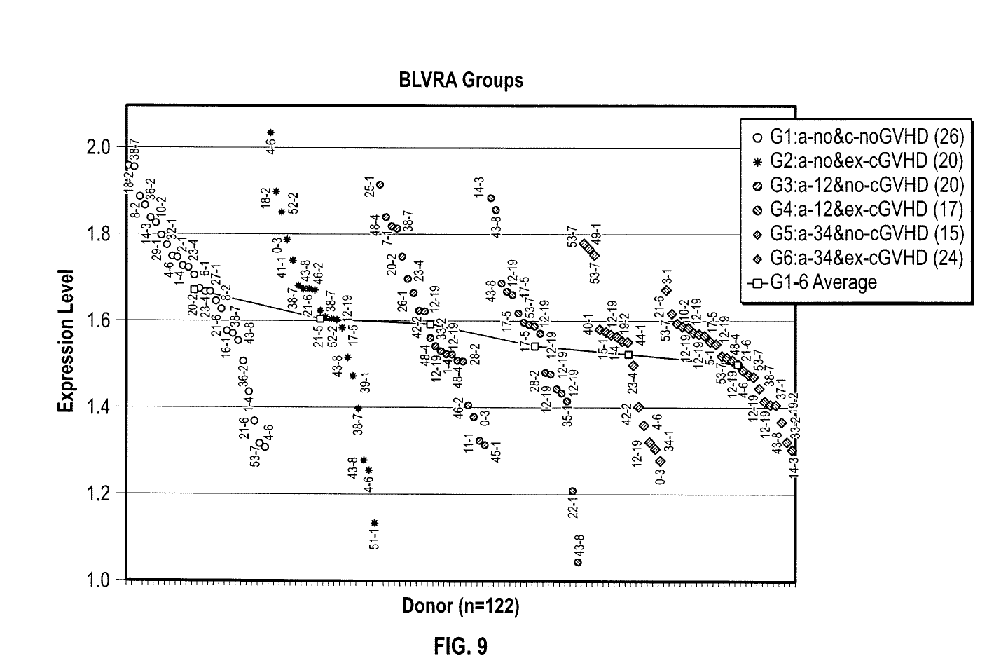

In another series of plots, samples are labeled according to the six GVHD outcome Groups, and the average RNA expression value for each Group is superimposed to specifically illustrate increasing or decreasing trends of gene expression with GVHD outcome Group number, and concomitant GVHD clinical intensity. In FIG. 8 (CTCF GROUPS) we observe a steady, monotonously increasing series of GVHD Group average with GVHD Group number. FIG. 9 (BLVRA GROUPS) illustrates a steady downward trend of GVHD Group average with GVHD Group number. Note the absence of very low, detection limit values in Group 1, and absence for very high values in Group 6, representing the most severe forms of GVHD.

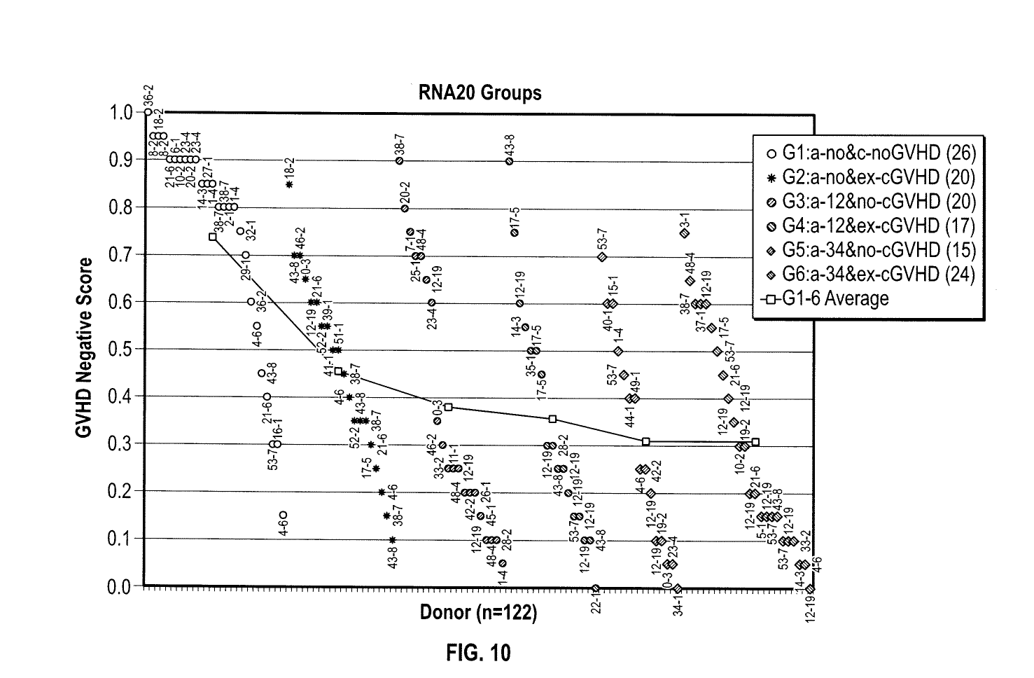

In FIG. 10 (RNA20 GROUPS), plotting the relative score of GVHD negative votes from 20 well-performing individual LDA models, there is a steady downward trend of GVHD Group average score with increasing GVHD severity. Notable is the much larger gap between the average of the no GVHD group (Group 1) and the averages of the five GVHD positive groups (Group 1 through Group 6) (Nomenclature Reminder: A class can comprise one Group, typically Group 1; another Class can comprise several Groups, e.g., the class representing “anyGVHD” comprises Groups 2 to 6).

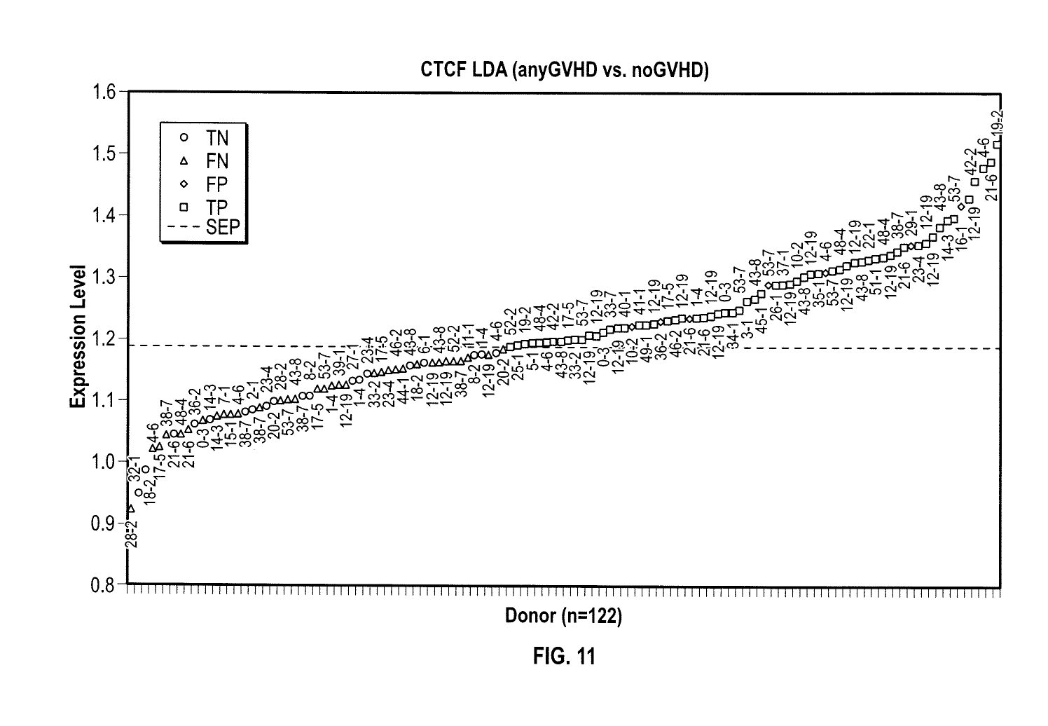

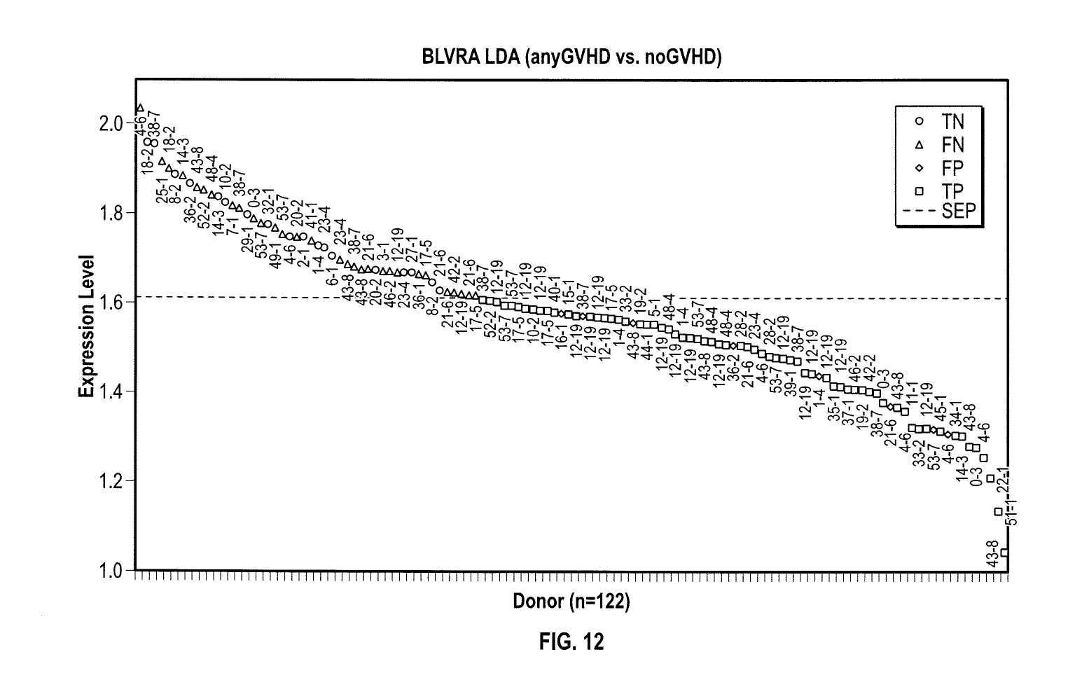

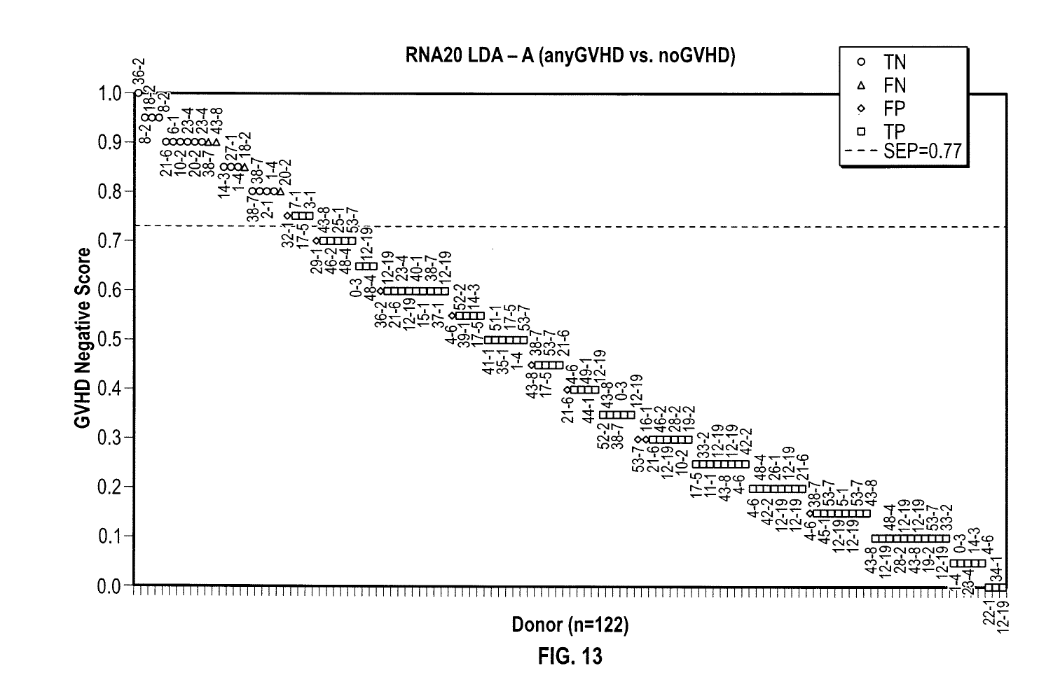

Sample-specific GVHD outcome prediction for anyGVHD vs. no GVHD (Table 6) is plotted in detail for the LDA models corresponding to the two individual RNA expression markers, and the 20 RNA marker voting model in FIGS. 11-13 (CTCF LDA, BLVRA LDA and RNA20 LDA-A). Essentially, samples are classified as being in the GVHD negative or positive class depending on whether their expression level falls on the same side (above or below) the separatrix as the average observed expression of that class, i.e. in FIG. 11 (CTCF LDA) samples are classified as GVHD negative below the separatrix, and in FIGS. 12 and 13 (BLVRA LDA and RNA 20 LDA) samples are classified as GVHD negative above the separatrix. Then depending on whether these classifications are correct, or incorrect, compared to the known, true class of the sample, each sample is then scored as either TN (True Negative), FN (False Negative), FP (False Positive), or TP (True Positive). As shown in FIGS. 11 and 12 (CTCF LDA, and BLVRA LDA), the vast majority of GVHD positive samples is classified correctly, but many of the negative samples are classified incorrectly (False Positives). Part of this asymmetry is a direct result of the overall asymmetrical representation of numbers of positive samples (n=96) and numbers negative samples (n=26) involved, i.e., even give a minor relative False Negative classification rate (False Negatives representing positive samples), this rate would be multiplied by 96/26=3.7 fold to arrive at the estimated observed number of positive samples misclassified as negative. Remarkably, in FIG. 13 (RNA20 LDA-A), even given the asymmetrical representation of positive and negative samples, there are only 4 GVHD positive samples misclassified as negative (NPV=0.94). This demonstrates that a simple voting scheme of 20 well-performing RNA expression LDA models is able to overcome most misclassifications that may be due to various sources of arbitrary signal variation and noise.

Note that the exemplary RNA20 voting scheme reflects a simple aggregation of GVHD negative predictions, i.e. votes, for each of the 20 component RNA marker RNA models, combined with a GVHD negative prediction threshold of “at least a 77% majority of the GVHD negative votes is required for a sample to be classified as GVHD negative” (see METHODS and Table 7).

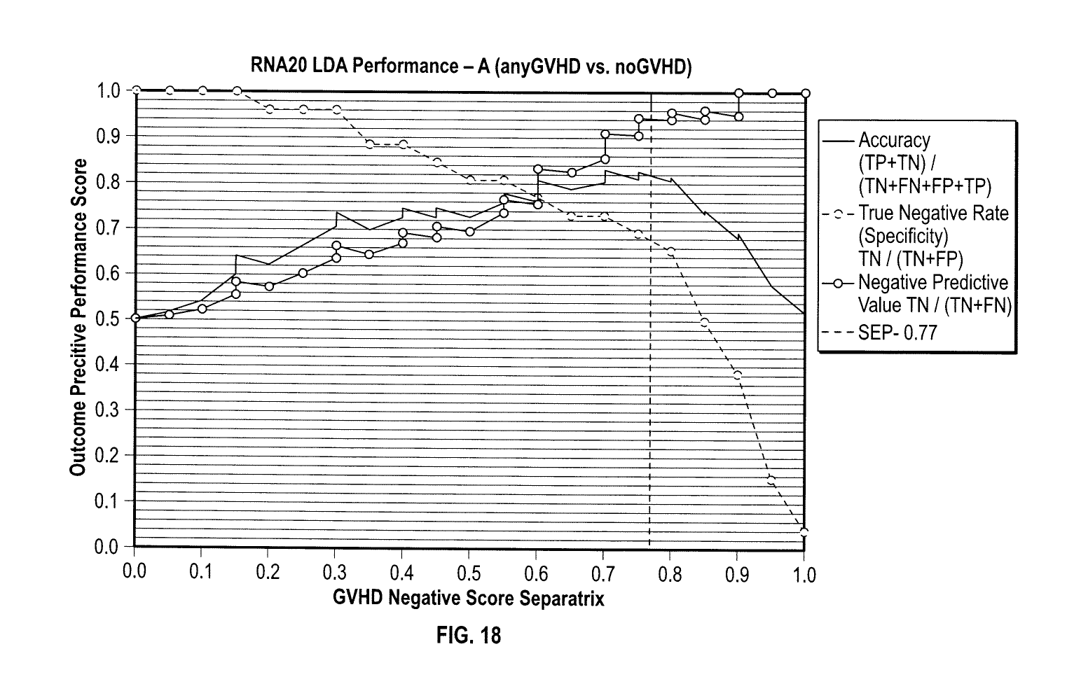

Essentially, the voting scheme is designed to overcome the limitations and error sources of each of the component markers by incorporating the information of 20 of them, and also provides flexibility in defining the stringency of GVHD negative outcome predictions through setting of the voting threshold. For example, the threshold value of 0.77 used here was manually selected to minimize False Negatives and maximize the Negative Predictive Value, while maintaining a relatively high number of True Negatives and high TNR (FIG. 18, RNA20 LDA PERFORMANCE-A). Correspondingly, in Table 7 very low numbers of False Negatives classified by the exemplary RNA20 model for each of the six GVHD groups was consistently observed. Note that for both Groups 5 and 6, totaling 39 samples originating from 24 different U.S. clinical centers, not a single False Negative prediction was observed. For clinical application, it is highly desirable to have low False Negative Rates, i.e., it is highly desirable to have a very low rate of declaring a donor suitable for HCT before transplantation so as to not induce GVHD, when in fact after transplantation, the patient unfortunately does present GVHD due to the donor HCT.

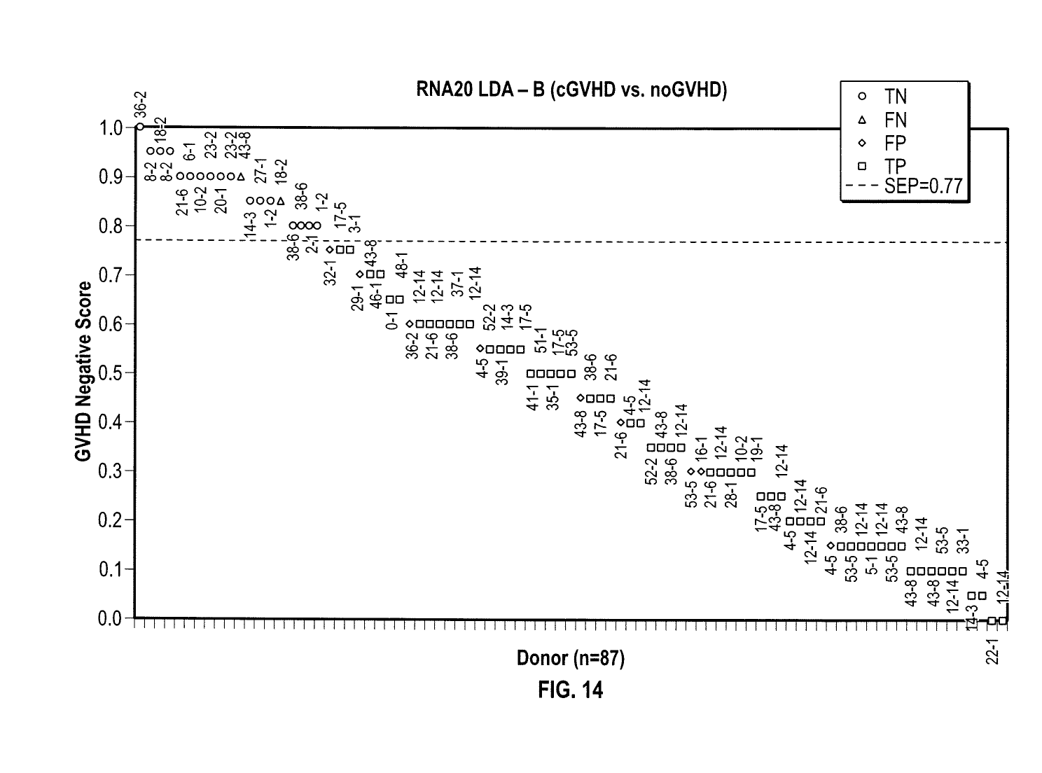

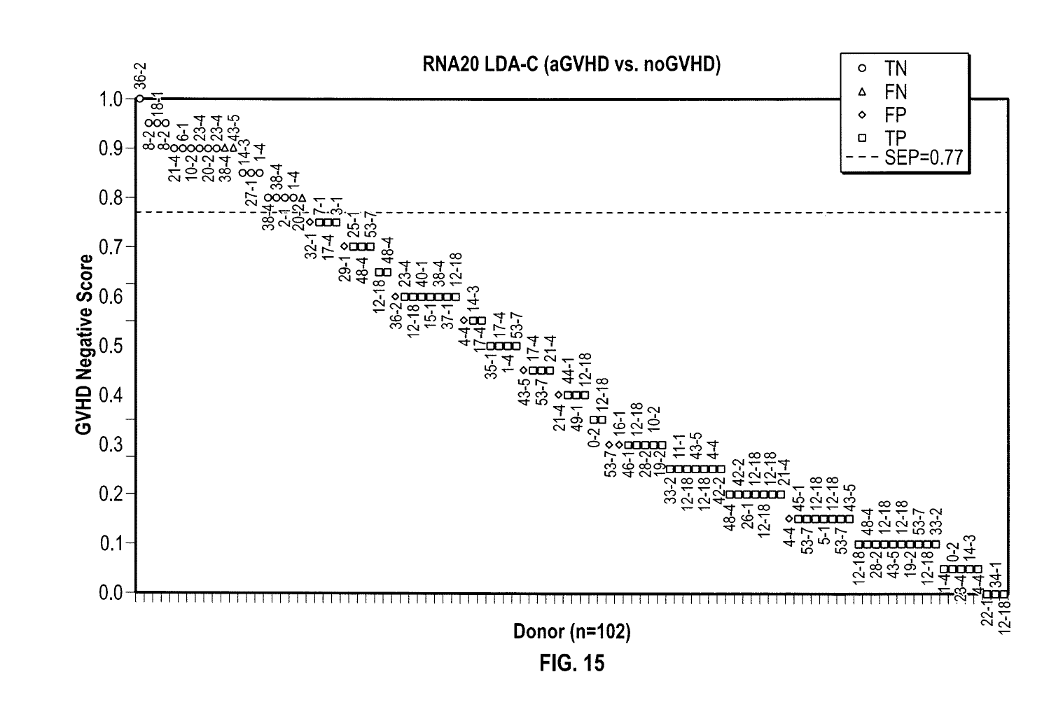

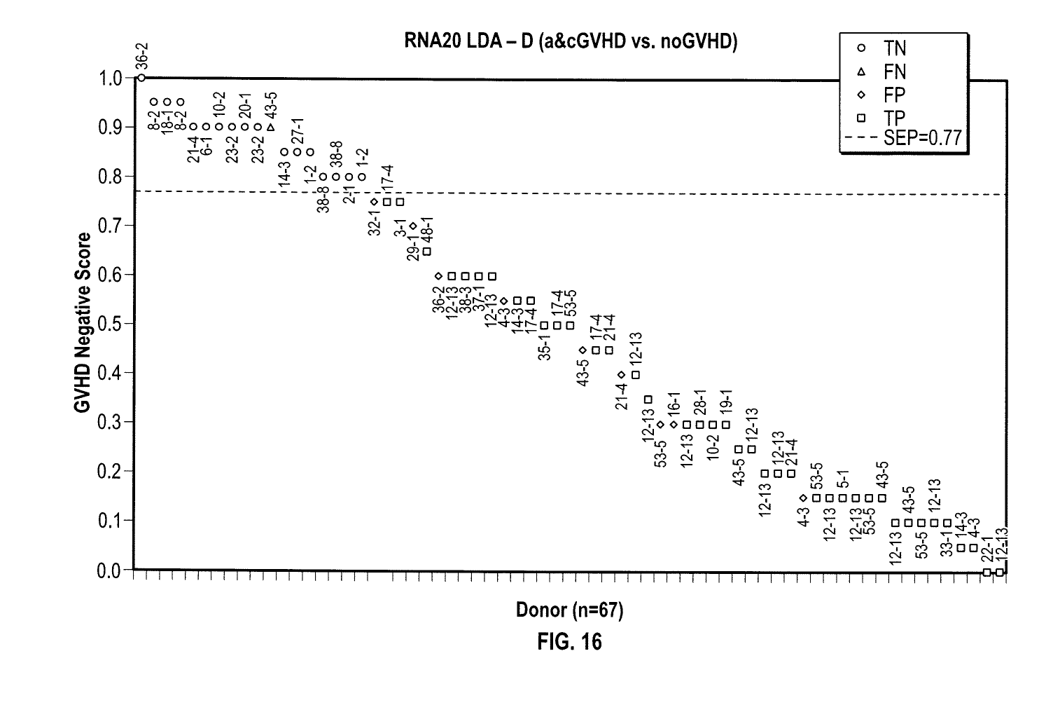

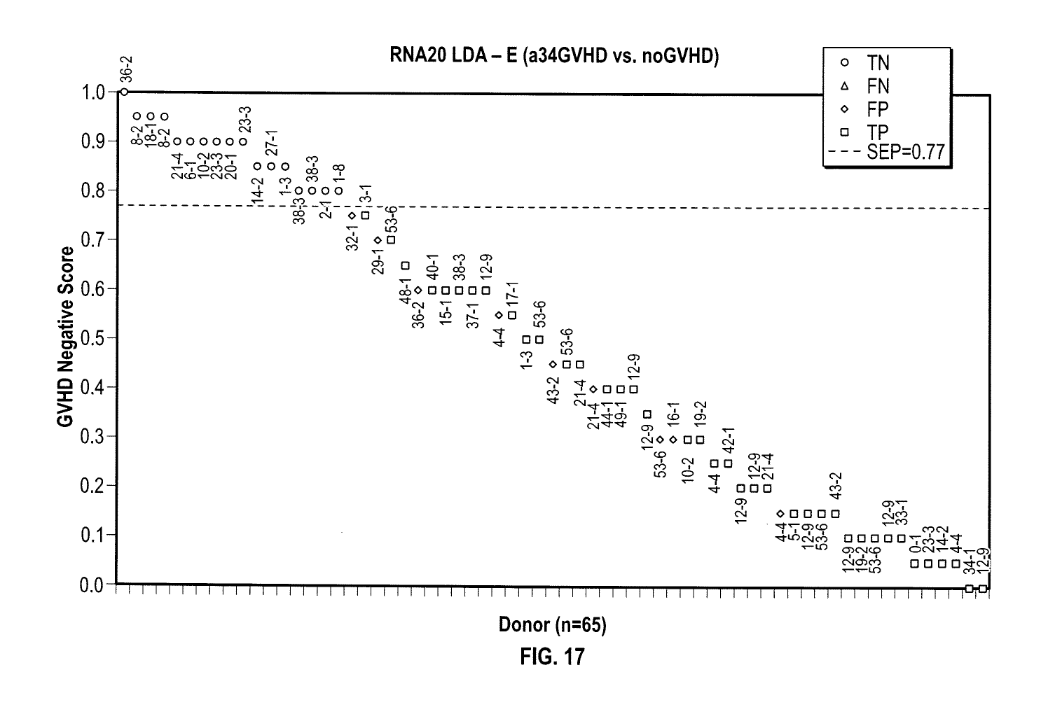

In addition to predicting well any GVHD vs. no GVHD outcomes, the 20 RNA expression LDA voting model also performs as well or better for distinguishing different types and intensities of chronic and acute GVHD from no GVHD outcomes (see Tables 6 and 11). For distinguishing chronic GVHD (alone or in combination with any form of acute GVHD) from no GVHD outcomes (cGVHD vs. noGVHD), only 2 False Negative classifications were reported (FIG. 14, RNA20 LDA-B) (NPV=0.95). For distinguishing any form of acute GVHD (alone or in combination with chronic GVHD) from no GVHD outcomes (aGVHD vs. noGVHD), only 3 False Negative classifications were reported (FIG. 15, RNA20 LDA-C) (NPV=0.94). For distinguishing chronic GVHD in combination with acute GVHD (in any form) from no GVHD outcomes (a&cGVHD vs. noGVHD), only 1 False Negative classification was reported (FIG. 16, RNA20 LDA-D) (NPV=0.96). Notably, for distinguishing the most severe forms of grade 3 or 4 acute GVHD (alone or in combination with chronic GVHD) from no GVHD outcomes (a34GVHD vs. noGVHD), not a single False Negative classification was reported (FIG. 17, RNA20 LDA-E).

### Example 7

This example includes a summary of GVHD outcome prediction performance.

Summary of GVHD Outcome Prediction Results:

The numerical outcome classification results for all of the single and 20 RNA marker models described above are summarized in Table 11. LDA outcome predictive measures are based on balanced LDA models, adjusted to represent an equal number of samples-wise contribution of GVHD negative and positive outcome samples (as described in Methods). While class discriminating T-test p-values of 6.6E-03 and 4.3E-03 (notation: xEy means standard scientific notation x times 10̂y) are reported for the single variable models for the anyGVHD vs. noGVHD division, the p-value for the 20RNA marker model is several orders of magnitude smaller, i.e. 1.1E-08. For the single RNA marker models, an overall Accuracy is reported of 69%, while for the 20 marker models, accuracies are much higher, in the 81-83% range.

Of the greatest potential clinical significance is the increase in the Negative Predictive Value (NPV) from 67-69% in the single marker models to >90% for the RNA20 voting models. The NPV represents how many of the samples that were classified as GVHD negative are truly negative. For example, using the data of this study, with an NPV of 100% for the a34GVHD vs. noGVHD outcome prediction, and if only donors were used that would be classified as GVHD negative using this model, none of such transplants would experience acute grade 3 or 4 GVHD. This would correspond to a complete, 100% elimination of GVHD occurrence based on the ˜50% GVHD incidence currently observed. This is a significant improvement over selecting HCT donors on the basis of HLA matching with an HCT recipient.

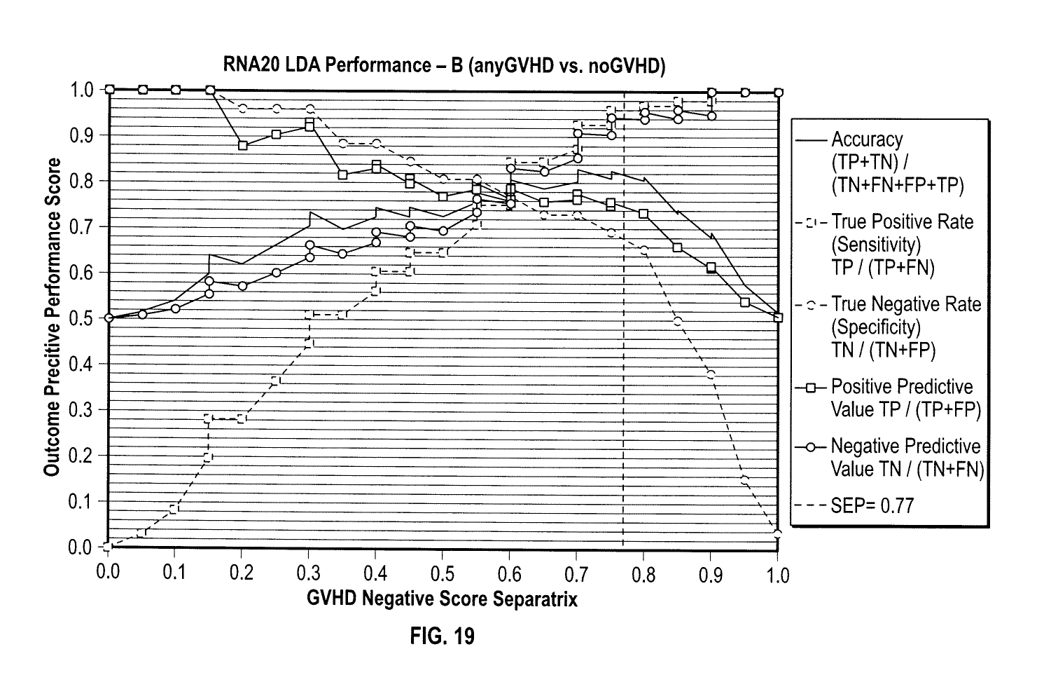

Trade-offs in outcome-prediction are possible through deliberate re-setting of thresholds (i.e., re-positionings of separatrices): When examining accuracy measures in LDA models for GVHD outcome prediction, gains in one performance measure may mean losses in another performance measure, depending on where the separatrix is positioned. For the exemplary RNA20 model, as the Negative Predictive Value (NPV) increases, the True Negative Rate (TNR, Specificity) decreases (FIG. 18, RNA20 LDA PERFORMANCE-A). Note: Such “detector threshold-dependent” tradeoff phenomena are well known generally and often summarized as “ROC” curves (Receiver Operating Characteristic curves). As more of the samples classified as GVHD negative turn out to be truly negative (NPV), the fewer of the total negative samples are classified correctly (TNR). In other words, the cost or price for minimizing the number of GVHD positive transplantations that are mistakenly classified before transplantation as negative, is that in a clinical context some donors leading to GVHD negative transplantations would be omitted. However, in the exemplary RNA20 model, in which the separatrix is positioned at a relative GVHD negative voting score of 0.77, a 94% NPV is obtained in combination with a 65% TNR. In other words, to be 94% certain to avoid GVHD, one would need to accept the loss of 35% of donors that would have been misclassified as GVHD positive. However, no substantial harm would be done, except for the loss of a candidate HCT donor. The detailed behavior of all 5 LDA accuracy measures, also including Positive Predict Value (PPV) and True Positive Rate (TPR, Sensitivity), is shown in FIG. 19, RNA20 LDA PERFORMANCE-B.

### Example 8

This example includes a description of using gene expression ratios to normalize or standardize values for comparison in GVHD outcome determination and prediction.

In analytic chemistry, physics, and quantitative measurement ratiometric assays can be more accurate than analogous assays employing only one analyte or measured quantity. An inherent accuracy advantage of a ratiometric assay, when it has an advantage, it that it is substantially self-calibrated against some reliable reference standard when the measurement is reported as a ratio of a signal of interest to a germane reference signal measured for the same sample using the same instrument.

Generally speaking, a ratiometric assay can involve (i) the ratio of a measured quantity A of interest divided by a measured reference value for A, (ii) the ratio of two distinct different (whether related or independent) measured quantities A and B, or more elaborate ratios such as, but not necessarily limited to, the ratio of A to a reference value of A divided by a ratio of B to a reference value of B, (iii) ratios of the kind described in points (i) and (ii) wherein the numerator and denominators are respectively differences between a measured signal and the measured background signal, (iv) combinations of more than one ratios of the kind described in points (i) through (iii).

The reliability of prediction or discrimination of two different outcomes is better when based on a ratiometric assay using a two analytes or predictors as a ratio rather than using two separate single-analyte assays. This phenomena of increased accuracy when in ratiometric form occurs when the contrast between two different outcomes (i.e., assessments or as predicted outcomes) is inherently enhanced when considering a ratio. For example, suppose outcome 1 archetype is characterized by the expression of gene A being high and the expression of gene B being low; whereas outcome 2 archetype is characterized by the expression of gene A being low and the expression of gene B being high. In situations of that kind, then typically the ratio of expressions A to B will be more accurate, and more sensitive, in discriminating one outcome vs. another, or in predicting one outcome rather than another, when the gene expression ratio is used rather than when using one gene alone or both genes separately.

Again, ratiometric assays of gene expression (i.e., gene expression ratios) can be formulated either as (i) a ratio of an expression of particular gene of interest (i.e., a predictor gene) relative to a housekeeping gene, or relative to a summary value assessed across a set of housekeeping genes, or (ii) as the ratio two different particular gene expressions of interest, i.e., as the ratio of two different predictor gene expressions. As explained above, version (i) has properties of intrinsic self-calibration of the measurement of the predictor, and (ii) has self-calibration properties and intrinsic increase of contrast relative to two different contrasting outcomes of interest (as described above).

One clear advantage of ratios over separate measurements, when it occurs, is when measurements are scaled by undetermined multiplicative factors, or gain, that are constant for a given instrument on which the measurements are made, then ratios are more accurate—especially with respective to differences from threshold values—than individual measurements. This is because a constant (albeit unknown) multiplicative scale factor cancels equally from both numerator and denominator in a ratio of two measurements.

There also are advantageous measurement noise-cancellation (technical, systemic, or biological noise or random variation) properties of ratios that are possible in some situations for ratios that are advantages not available to single gene (single predictor) measurements used separately. In particular, if on the average the expression of two different genes are anti-correlated, (i.e., when one tends to increase, the other tends to decrease, or vice-versa), then when re-cast in logarithmic form (or logarithmic-like form, e.g., inverse hyperbolic sine transformed), then the quasi-random noise components of the two separate gene expression measurements tend to cancel when the two gene expression measurements are fundamentally composed as a ratio. This is a general mathematical, or arithmetic, or statistical phenomenon (i.e., not confined only to gene expression) sometimes known as “the method of antithetic variables” (C Eisenhart & M. Zelin, Elements of Probability, Ch. 12, in EU Condon & H Odishaw, eds., Handbook of Physics, McGraw-Hill Book Co., NY, 1958, pp. 1-143; P. Kevin MacKeown, Stochastic Simulation in Physics, Springer-Verlag, Singapore, 1997, esp. p. 21 & p. 212) where in the sum of two variables has less noise or variance than the sum of the noise or variance of each variable separately when the two variables are anti-correlated.

When logarithm, or a quasi-log functions such as inverse hyperbolic sine, of a ratio is used, then the log ratio becomes the log of the numerator minus the log of the denominator. If the numerator and denominator variables are more or less negatively correlated, then the “antithetic variables” partially cancel noise, or a reduction in variance occurs. That is, the phenomenon of partial cancellation of noise through “antithetic variables” is an additional side-advantage that can be obtained when ratiometric measurements are employed.

### Example 9

This example includes a description of prediction of GVHD risk on a continuous scale or score or index, based on measured expression levels of single or multiple predictor genes.

Gene expression measurements of predictor genes, or expression ratios involving predictor genes and a reference gene (e.g., housekeeping gene), or expression ratios of two different predictor genes in HCT donors represent in principle a continuum of numerical values. Each such measurement values, or interval or values, or range of values (from the continuum) can be associated, in an experimentally \computationally \statistically evidence-based way, with a particular predicted risk before transplantation that the donor's HCT will induce GVHD, or not induce GVHD, in the HCT recipient (patient) after transplantation.

A single example of such a risk of GVHD number or value could be a threshold reference expression measurement value, below or above which, the risk of GVHD occurring will be below or above a certain probability, or vice-versa; which in turn can be described as low or high risk of GVHD. GVHD outcome prediction essentially can be then carried out in two ways: (1) as reporting a high or low GVHD risk, or reporting a specific probability or interval or range of probability; or (2) as a value on a continuum of GVHD probabilities or GVHD risk scores. Such risk scores, which are considered to be coming from a continuum, under a GVHD outcome predictive mathematical\statistical \numerical model applied to measured gene expressions could be implemented through use of straight-forward mathematical formulas or from pre-computed numerical look-up tables that capture the same numerical input (gene expressions)-to-output (risk of GVHD) mappings or behaviors as would a mathematical formula.

Moreover, measured expression values involving multiple different genes, or multiple expression ratios, may be combined further in multiple ways—such as simple arithmetic addition or addition with re-scaling by pre-defined constants, or other straight-forward and clear mathematical operations, to arrive at a continuous-valued output variable that can be associated with a continuum of GVHD risk, or GVHD probability, or GVHD risk scores, etc., or indeed threshold reference values for specific defined GVHD risks or GVHD probabilities.

Also, expression measurement values of GVHD outcome predictive genes, or ratios involving such predictive genes and reference (e.g., housekeeping) genes, or ratios involving two different genes, each separately can cast a high-GVHD-risk vote (e.g., numerical value=1 or a little less than 1), or a low-GVHD-risk vote (e.g., numerical value=0 or little larger than 0), and these votes from a set of such measurements or ratios of measurements can be added together to form an overall voting score or index. The voting score or index can be considered to be form a continuum, or quasi-continuum, ranging between 0 and 1. Such a voting score of index then can be associated with a continuum of GVHD risks or GVHD probabilities, or fall above or below certain pre-defined threshold values for likely or unlikely GVHD, or fall into pre-defined intervals (partitioning the score range between 0 and 1) that qualitatively report degrees of GVHD risk.

### Example 10

This example includes a description of the analysis of an additional 120 donor PBMC (peripheral blood mononuclear cells) samples, combined with the 122 donor PBMC samples described above, resulting in a total of 242 donor PBMC samples, with corresponding recipient GVHD histories to identify GVHD predictor genes.

Conventional computational cross-validation was applied as an approach to assess the outcome predictive performance of single genes and voting schemes. To assist ranking genes among one another for outcome predictive capability, a form of constrained linear discriminant analysis was employed as well to assess genes' performance on discriminating different degrees of GVHD vs. no GVHD across the set of patients associated with their respective donor.

As a result of the advanced computational statistical analysis listed above having been carried out on a total of 242 donor sample gene expression profiles with GVHD outcome information (120 new donor samples added to the initial 122 donor samples analyzed as discussed in detail above), an additional set of 121 genes were identified as GVHD outcome predictors. In addition, 23 genes were identified as housekeeping (“HSK”) genes (including the eukaryotic translation initiation factor 4H (EIF4H) transcript variant 1 gene previously listed as a predictive gene based on analysis of the initial 122 samples). In total, 143 new genes (Table 2A, RNA143) were identified as diagnostic test candidate genes (121 outcome predictor genes, 22 housekeeping genes, all also fully listed in the RNA192 list, Table 2B), that were not included in the results of the initial analysis of 122 samples and therefore not included in the prior RNA1546 list. As a result of a more in-depth analysis of the complete 242 donor sample/GVHD history dataset, 192 genes (Table 2B, 169 predictive genes and 23 housekeeping genes) have now been selected as an exemplary “RNA192” list of the genes for further high fidelity RT-PCR gene expression assays.

### Example 11

This example includes a description of real-time, reverse transcription (RT) quantitative polymerase chain reaction (PCR) measurement of candidate N and P predictor gene expression, and N and P predictor gene expression data.

For applications in human medical diagnostics, RT-PCR (Reverse Transcription Polymerase Chain Reaction) for gene expression measurement, such as implemented in the TaqMan real-time RT-PCR platform (ABI, Applied Biosystems Inc.), is considered to be the “gold standard” for high-fidelity quantitative gene expression level assessment, compared to the generally deemed to be less accurate and less sensitive microarray gene expression analysis (such as used for the survey of mRNA levels described herein from GVHD donor samples using the IIlumina HT12 v3.0 microarray platform covering ˜48,000 different gene-specific probes).

Real-time RT-PCR (Reverse Transcription Polymerase Chain Reaction) gene expression data was acquired for 192 specified genes (listed in Table 2B as “RNA 192”) and 180 different donor frozen blood samples. Conventional TaqMan platform was employed, using 100 ng cDNA per reaction (cDNA being derived from the RNA samples acquired as described above). Each gene is defined by a unique primer according to an off-the-shelf TaqMan assay ID (ABI, Applied Biosystems Inc.). The technically pre-validated commercially available TaqMan assays define the nucleotide sequences of the gene-specific primers and hydrolysis probes; however, the exact nucleotide sequences are proprietary to ABI. ABI TaqMan assays have been optimized by ABI to result in a PCR amplification efficiency (E) of E=2.

The following is conventional and an industrial standard, essentially in commoditized use for many years: RT-PCR involves an initial RT (reverse transcription) step, which converts the RNA to cDNA, which is followed by PCR (polymerase chain reaction) amplification of the cDNA. Real-time RT-PCR involves the use of gene sequence specific internal hydrolysis probe, in addition to the gene sequence-specific primers. The internal hydrolysis probe contains nucleotides that are chemically modified with fluorescence probes, which deliberately quench each other's fluorescence when placed in close proximity on the hydrolysis probe strand. When the internal hydrolysis probe binds to a single strand of amplified, gene specific cDNA, the 5′-3′ exonuclease activity of the thermo-stable DNA polymerase used in PCR breaks up the hydrolysis probe into its constituent nucleotides. As these fluorescently labeled constituent nucleotides are released, they no longer quench each other, and a quantifiable fluorescence signal proportional to the gene-specific mRNA copy number (but as cDNA starting amount) emerges in the RT-PCR reaction. This signal increases proportionally in a gene-specific fashion to the amount of cDNA amplified.

The TaqMan measurement output reports for each sample and each gene a Cₜ value, defined as the RT-PCR cycle number at which a pre-defined fluorescence signal (STV, signal threshold value) of the assay is achieved. All instrument-level Cₜ value measurement output data will from now on be referred to here as RWCT, i.e. “raw Cₜ”. The RWCT value is inversely proportional to the concentration of the starting amount of the cDNA (because the smaller the initial cDNA starting amount, the more RT-PCR cycles, i.e., the greater is Cₜ, necessary to reach STV).

Determining the original amount of starting material before PCR amplification, i.e., S (signal), is carried out according to the following literature and established in-practice equation, expressed in logarithmic form (log is always defined as log₁₀, i.e. logarithm on base 10):

log S=log STV−Cₜ*log E

where:
S=Initial signal or amount of gene in sample (which is to be imputed by the assay);
STV=Signal Threshold Value of gene amplification for determining Cₜ (predefined); and
E=Efficiency of PCR amplification (thoroughly checked by PBD to be essentially equal to 2).

The term “UNDETERMINED” is assigned conventionally for RWCT values >40 in the TaqMan output, since signals above 40 cycles are not considered reliable, i.e., too many amplification cycles are needed to reach STV. Standard real-time RT-PCR practice terminates the amplification procedure at 40 cycles.

For the analysis used herein, RWCT values <20 are labeled as OUTLIERS, since they are suggestive of unrealistically high gene-specific initial amounts that therefore should not be considered as a reliable assay output.

As implemented herein, RT-PCR data pre-processing in 4 steps to arrive at RRCF values, on which GVHD outcome prediction determinations are based. Note: RRCF is defined as “RT-PCR, Relative, log signal, E=2, Clear, with Floor values replaced”.

1. Replacement of OUTLIER values:
RCTC (RT-PCR, Cₜ, Clean)
All OUTLIER values are replaced with the median RWCT value of the gene over all 180 samples in the dataset. “Clean” refers to OUTLIER values having been substituted with the median.
   2. Generation of logarithmic RT-PCR signal for PCR efficiency E=2:
RL2C (RT-PCR, Log signal, E=2, Clean)
RL2C values are calculated from RCTC values according to the following equation:

RL2C=log STV−RCTC*log(2)
Note: log STV=14, i.e. log STV is defined by PBD to equal 14 in arbitrary units (there is no existing convention in RT-PCR practice that recommends a particular unit or scaling), in an effort to generate RT-PCR output values to be in a numerical range comparable to the processed Illumina microarray data leading the GVHD predictive gene lists in the P2 filing.
   3. Replacement of UNDETERMINED “floor” values:
RL2F (RT-PCR, Log signal, E=2, clean, Floor values replaced)
UNDETERMINED “floor” values are substituted by the following value (representing the RL2C value for RCTC=40):

RL2F(for UNDETERMINED values)=[14−40*log(2)]=1.95880017344075

4. Relative (relative quantitation) RT-PCR signal through correction of background signal by subtraction of relative average signal of housekeeping (HSK) genes:
RRCF (RT-PCR, Relative, log signal, E=2, Clean, Floor values replaced)
Relative quantitation of the RT-PCR signal through correction of background signal is carried out by subtraction of the relative average signal of 6 housekeeping (RHSKAG6) genes of each sample according to the following equation:

RRCF=RL2F−RHSKAG6
Note: RHSKAG6 is defined for each sample as the Relative HSK Average signal of 6 PBD-selected HSK genes (HSK6), centered at zero over all 180 RHSKAG6 values:

RHSKAG6=HSKAG6−AVGHSKAG6
AVGHSKAG6 is defined as a constant, representing the average value of all 180 sample-specific determinations of HSKAG6.
HSKAG6 is defined for each sample as the average of the 6 housekeeping gene RL2F values.

Definition of Exemplary 6 Housekeeping Genes (HSK6) for Determination of HSKAG6 Value Used for RRCF Determination:

Table 12 details the HSK6 list, used for HSKAG6 and RHSKAG6 determination (also see above):

RHSKAG6=HSKAG6−[average HSKAG6 over all 180 samples]

TABLE 12
List of 6 housekeeping genes (“HSK6”) used for relative quantitation of RRCF RT-PCR signal

| | | | | | | P2 PBD | |
|---|---|---|---|---|---|---|---|
| Gene 175 | RNA192 | ACCESSION BASIC | | ABI Gene | | performance | P2 Dx |
| index | Index | (without decimal) | ABI Assay ID | Symbol | ABI Gene Name | rank | function |
| 158 | 175 | NM_032195 | Hs00371372_m1 | SON | SON DNA binding protein | 06HSK | HSK |
| 159 | 176 | NM_016061 | Hs00763191_s1 | YPEL5 | yippee-like 5 (Drosophila) | 07HSK | HSK |
| 160 | 177 | NM_013379 | Hs01115161_m1 | DPP7 | dipeptidyl-peptidase 7 | 08HSK | HSK |
| 162 | 179 | NM_001033112 | Hs00212868_m1 | PAIP2 | poly(A) binding protein | 11HSK | HSK |
| | | | | | interacting protein 2 | | |
| 166 | 183 | NM_018064 | Hs00363236_m1 | AKIRIN2 | akirin 2 | 15HSK | HSK |
| 173 | 190 | NM_030914 | Hs00229455_m1 | URM1 | ubiquitin related modifier 1 | 23HSK | HSK |

Selection of 175 Genes that Meet Initial QC Criteria from Total Set of 192 Genes in Table 2B:

The definition herein for the fundamental QC (quality control) criterion that a gene must have been detected (i.e. not have UNDETERMINED, RWCT>=40 values) in >=55% of the data samples. Table 13 lists the 175 genes (“SG175”) of the total set of 192 genes listed in Table 2B that may be considered for further analysis.

TABLE 13
List of exemplary 175 genes (SG175) from total set of 192 genes that meet detectability QC criterion

| | Table 2B, | ACCESSION | | ABI | | P2 PBD | | |
|---|---|---|---|---|---|---|---|---|
| Gene 175 | RNA192 | BASIC | ABI | Gene | ABI | performance | P2 Dx | P2 |
| index | Index | (without decimal) | Assay ID | Symbol | Gene Name | rank | function | Direction |
| 1 | 1 | NM_030938 | Hs00229548_m1 | TMEM49 | transmembrane protein 49 | 001N | PRD | N |
| 2 | 2 | NM_014232 | Hs00360269_m1 | VAMP2 | vesicle-associated membrane | 001P | PRD | P |
| | | | | | protein 2 (synaptobrevin 2) | | | |
| 3 | 3 | NM_000024 | Hs00240532_s1 | ADRB2 | adrenergic, beta-2-, receptor, | 002N | PRD | N |
| | | | | | surface | | | |
| 4 | 4 | NM_004538 | Hs00270173_s1 | NAP1L3 | nucleosome assembly protein 1- | 002P | PRD | P |
| | | | | | like 3 | | | |
| 5 | 5 | NM_001018069 | Hs00967385_g1 | SERBP1 | SERPINE1 mRNA binding | 003N | PRD | N |
| | | | | | protein 1 | | | |
| 6 | 6 | NM_015989 | Hs00211126_m1 | CSAD | cysteine sulfinic acid | 003P | PRD | P |
| | | | | | decarboxylase | | | |
| 7 | 7 | NM_017455 | Hs00247361_m1 | NPTN | neuroplastin | 004N | PRD | N |
| 8 | 8 | NM_001129 | Hs00937468_m1 | AEBP1 | AE binding protein 1 | 004P | PRD | P |
| 9 | 9 | NM_021601 | Hs00233566_m1 | CD79A | CD79a molecule, | 005N | PRD | N |
| | | | | | immunoglobulin-associated | | | |
| | | | | | alpha | | | |
| 10 | 10 | NM_000997 | Hs02340038_g1 | RPL37 | ribosomal protein L37 | 005P | PRD | P |
| 11 | 11 | NM_145869 | Hs00175132_m1 | ANXA11 | annexin A11 | 006N | PRD | N |
| 12 | 12 | NM_006297 | Hs00959834_m1 | XRCC1 | X-ray repair complementing | 006P | PRD | P |
| | | | | | defective repair in Chinese | | | |
| | | | | | hamster cells 1 | | | |
| 13 | 13 | NM_024921 | Hs00227769_m1 | POF1B | premature ovarian failure, 1B | 007N | PRD | N |
| 14 | 14 | NM_001003789 | Hs00255244_m1 | RABL2B | RAB, member of RAS oncogene | 007P | PRD | P |
| | | | | | family-like 2B, RAB, member of | | | |
| | | | | | RAS oncogene family-like 2A | | | |
| 15 | 15 | NM_030918 | Hs00229472_m1 | SNX27 | sorting nexin family member 27 | 008N | PRD | N |
| 16 | 16 | NM_080430 | Hs00369741_m1 | SELM | selenoprotein M | 008P | PRD | P |
| 17 | 17 | NM_005437 | Hs01033772_g1 | NCOA4 | nuclear receptor coactivator 4 | 009N | PRD | N |
| 18 | 18 | NM_002129 | Hs01127828_g1 | HMGB2 | high-mobility group box 2 | 009P | PRD | P |
| 19 | 20 | NM_152468 | Hs00380060_m1 | TMC8 | transmembrane channel-like 8 | 010P | PRD | P |
| 20 | 21 | NM_133471 | Hs00292978_m1 | KIAA1949 | KIAA1949 | 011N | PRD | N |
| 21 | 22 | NM_138923 | Hs00270322_m1 | TAF1 | TAF1 RNA polymerase II, | 011P | PRD | P |
| | | | | | TATA box binding protein | | | |
| | | | | | (TBP)-associated factor, 250 kDa | | | |
| 22 | 23 | NM_018367 | Hs00218034_m1 | ACER3 | alkaline ceramidase 3 | 012N | PRD | N |
| 23 | 24 | NM_201554 | Hs00176278_m1 | DGKA | diacylglycerol kinase, alpha | 012P | PRD | P |
| | | | | | 80 kDa | | | |
| 24 | 25 | NM_145755 | Hs00377534_m1 | TTC21A | tetratricopeptide repeat domain | 013N | PRD | N |
| | | | | | 21A | | | |
| 25 | 26 | NM_018571 | Hs00251360_s1 | STRADB | STE20-related kinase adaptor | 013P | PRD | P |
| | | | | | beta | | | |
| 26 | 28 | NM_002811 | Hs00427396_m1 | PSMD7 | proteasome (prosome, | 014P | PRD | P |
| | | | | | macropain) 26S subunit, non- | | | |
| | | | | | ATPase, 7 | | | |
| 27 | 29 | NM_020654 | Hs00221046_m1 | SENP7 | SUMO1/sentrin specific | 015N | PRD | N |
| | | | | | peptidase 7 | | | |
| 28 | 30 | NM_002082 | Hs00357776_g1 | GRK6 | G protein-coupled receptor | 015P | PRD | P |
| | | | | | kinase 6 | | | |
| 29 | 31 | NM_001042472 | Hs01018047_m1 | ABHD12 | abhydrolase domain containing | 016N | PRD | N |
| | | | | | 12 | | | |
| 30 | 32 | NM_004698 | Hs00757030_m1 | PRPF3 | PRP3 pre-mRNA processing | 016P | PRD | P |
| | | | | | factor 3 homolog (S. cerevisiae) | | | |
| 31 | 34 | NM_153701 | Hs00538167_m1 | IL12RB1 | interleukin 12 receptor, beta 1 | 017P | PRD | P |
| 32 | 35 | NM_012459 | Hs02339636_g1 | TIMM8B | translocase of inner | 018N | PRD | N |
| | | | | | mitochondrial membrane 8 | | | |
| | | | | | homolog B (yeast) | | | |
| 33 | 36 | NM_001077268 | Hs00262564_m1 | ZFYVE19 | zinc finger, FYVE domain | 018P | PRD | P |
| | | | | | containing 19 | | | |
| 34 | 37 | NM_006371 | Hs01035151_m1 | CRTAP | cartilage associated protein | 019N | PRD | N |
| 35 | 38 | NR_003654 | Hs00364437_m1 | SCAND2 | SCAN domain containing 2 | 019P | PRD | P |
| | | | | | pseudogene | | | |
| 36 | 39 | NM_006762 | Hs00198882_m1 | LAPTM5 | lysosomal protein | 020N | PRD | N |
| | | | | | transmembrane 5 | | | |
| 37 | 40 | NM_016619 | Hs00930964_g1 | PLAC8 | placenta-specific 8 | 020P | PRD | P |
| 38 | 41 | NM_003780 | Hs00243566_m1 | B4GALT2 | UDP-Gal:betaGlcNAc beta 1,4- | 021N | PRD | N |
| | | | | | galactosyltransferase, | | | |
| | | | | | polypeptide 2 | | | |
| 39 | 43 | NM_024896 | Hs00227643_m1 | ERMP1 | endoplasmic reticulum | 022N | PRD | N |
| | | | | | metallopeptidase 1 | | | |
| 40 | 44 | NM_002494 | Hs00159587_m1 | NDUFC1 | NADH dehydrogenase | 022P | PRD | P |
| | | | | | (ubiquinone) 1, subcomplex | | | |
| | | | | | unknown, 1, 6 kDa | | | |
| 41 | 45 | NM_000161 | Hs00609198_m1 | GCH1 | GTP cyclohydrolase 1 | 023N | PRD | N |
| 42 | 46 | NM_006346 | Hs00197131_m1 | PIBF1 | progesterone | 023P | PRD | P |
| | | | | | immunomodulatory binding | | | |
| | | | | | factor 1 | | | |
| 43 | 47 | NM_145288 | Hs00377132_m1 | ZNF296 | zinc finger protein 296 | 024N | PRD | N |
| 44 | 48 | NM_016446 | Hs00255552_m1 | TMEM8B | transmembrane protein 8B | 024P | PRD | P |
| 45 | 50 | NM_012117 | Hs01127577_m1 | CBX5 | chromobox homolog 5 | 025P | PRD | P |
| 46 | 51 | NM_130787 | Hs00367123_m1 | AP2A1 | adaptor-related protein complex | 026N | PRD | N |
| | | | | | 2, alpha 1 subunit | | | |
| 47 | 52 | NM_001981 | Hs00179978_m1 | EPS15 | epidermal growth factor receptor | 026P | PRD | P |
| | | | | | pathway substrate 15 | | | |
| 48 | 53 | NM_005871 | Hs00195343_m1 | SMNDC1 | survival motor neuron domain | 027N | PRD | N |
| | | | | | containing 1 | | | |
| 49 | 54 | NM_022474 | Hs00223885_m1 | MPP5 | membrane protein, palmitoylated | 027P | PRD | P |
| | | | | | 5 (MAGUK p55 subfamily | | | |
| | | | | | member 5) | | | |
| 50 | 55 | NM_145912 | Hs00377608_m1 | NFAM1 | NFAT activating protein with | 028N | PRD | N |
| | | | | | ITAM motif 1 | | | |
| 51 | 56 | NM_016173 | Hs00275076_m1 | HEMK1 | HemK methyltransferase family | 028P | PRD | P |
| | | | | | member 1 | | | |
| 52 | 57 | NM_173843 | Hs00893626_m1 | IL1RN | interleukin 1 receptor antagonist | 029N | PRD | N |
| 53 | 58 | NM_006565 | Hs00902008_m1 | CTCF | CCCTC-binding factor (zinc | 029P | PRD | P |
| | | | | | finger protein) | | | |
| 54 | 59 | NM_000201 | Hs00164932_m1 | ICAM1 | intercellular adhesion molecule 1 | 030N | PRD | N |
| 55 | 60 | NM_145306 | Hs00293954_m1 | C10orf35 | chromosome 10 open reading | 030P | PRD | P |
| | | | | | frame 35 | | | |
| 56 | 61 | NM_005360 | Hs00193519_m1 | MAF | v-maf musculoaponeurotic | 031N | PRD | N |
| | | | | | fibrosarcoma oncogene homolog | | | |
| | | | | | (avian) | | | |
| 57 | 62 | NM_001459 | Hs00181740_m1 | FLT3LG | fms-related tyrosine kinase 3 | 031P | PRD | P |
| | | | | | ligand | | | |
| 58 | 63 | NM_015112 | Hs00248380_m1 | MAST2 | microtubule associated | 032N | PRD | N |
| | | | | | serine/threonine kinase 2 | | | |
| 59 | 64 | NM_015057 | Hs00209335_m1 | MYCBP2 | MYC binding protein 2 | 032P | PRD | P |
| 60 | 66 | NM_201438 | Hs00212889_m1 | PPHLN1 | periphilin 1 | 033P | PRD | P |
| 61 | 68 | NM_004798 | Hs01122781_m1 | KIF3B | kinesin family member 3B | 034P | PRD | P |
| 62 | 70 | NM_152850 | Hs00912503_m1 | PIGO | phosphatidylinositol glycan | 035P | PRD | P |
| | | | | | anchor biosynthesis, class O | | | |
| 63 | 71 | NM_004155 | Hs00244603_m1 | SERPINB9 | serpin peptidase inhibitor, clade | 036N | PRD | N |
| | | | | | B (ovalbumin), member 9 | | | |
| 64 | 72 | NM_003328 | Hs01053640_m1 | TXK | TXK tyrosine kinase | 036P | PRD | P |
| 65 | 73 | NM_020820 | Hs00368207_m1 | PREX1 | phosphatidylinositol-3,4,5- | 037N | PRD | N |
| | | | | | trisphosphate-dependent Rac | | | |
| | | | | | exchange factor 1 | | | |
| 66 | 74 | NM_001007468 | Hs00268260_m1 | SMARCB1 | SWI/SNF related, matrix | 037P | PRD | P |
| | | | | | associated, actin dependent | | | |
| | | | | | regulator of chromatin, | | | |
| | | | | | subfamily b, member 1 | | | |
| 67 | 75 | NM_018044 | Hs00216128_m1 | NSUN5 | NOP2/Sun domain family, | 038N | PRD | N |
| | | | | | member 5 | | | |
| 68 | 76 | NM_172177 | Hs00204112_m1 | MRPL42 | mitochondrial ribosomal protein | 038P | PRD | P |
| | | | | | L42 | | | |
| 69 | 77 | NM_020808 | Hs00384853_m1 | SIPA1L2 | signal-induced proliferation- | 039N | PRD | N |
| | | | | | associated 1 like 2 | | | |
| 70 | 79 | NM_006007 | Hs00829622_s1 | ZFAND5 | zinc finger, AN1-type domain 5 | 040N | PRD | N |
| 71 | 80 | NM_013374 | Hs00183813_m1 | PDCD6IP | programmed cell death 6 | 040P | PRD | P |
| | | | | | interacting protein | | | |
| 72 | 81 | NM_001014839 | Hs00379444_m1 | NCDN | neurochondrin | 041N | PRD | N |
| 73 | 82 | NM_006370 | Hs00762282_s1 | VTI1B | vesicle transport through | 041P | PRD | P |
| | | | | | interaction with t-SNAREs | | | |
| | | | | | homolog 1B (yeast) | | | |
| 74 | 84 | NM_032026 | Hs00757279_mH | TATDN1 | TatD DNase domain containing 1 | 042P | PRD | P |
| 75 | 85 | NM_005436 | Hs00193731_m1 | CCDC6 | coiled-coil domain containing 6 | 043N | PRD | N |
| 76 | 86 | NM_032314 | Hs00260456_m1 | COQ5 | coenzyme Q5 homolog, | 043P | PRD | P |
| | | | | | methyltransferase (S. cerevisiae) | | | |
| 77 | 87 | NM_002158 | Hs00939664_m1 | FOXN2 | forkhead box N2 | 044N | PRD | N |
| 78 | 88 | NM_007124 | Hs01126016_m1 | UTRN | utrophin | 044P | PRD | P |
| 79 | 89 | NM_138711 | Hs01115513_m1 | PPARG | peroxisome proliferator- | 045N | PRD | N |
| | | | | | activated receptor gamma | | | |
| 80 | 90 | NM_019083 | Hs00219487_m1 | CCDC76 | coiled-coil domain containing 76 | 045P | PRD | P |
| 81 | 91 | NM_001002246 | Hs00212858_m1 | ANAPC11 | anaphase promoting complex | 046N | PRD | N |
| | | | | | subunit 11 | | | |
| 82 | 92 | NM_001007277 | Hs00903035_g1 | EI24 | etoposide induced 2.4 mRNA | 046P | PRD | P |
| 83 | 93 | NM_004450 | Hs00427977_m1 | ERH | enhancer of rudimentary | 047N | PRD | N |
| | | | | | homolog (Drosophila) | | | |
| 84 | 94 | NM_032449 | Hs00383486_m1 | CC2D1B | coiled-coil and C2 domain | 047P | PRD | P |
| | | | | | containing 1B | | | |
| 85 | 95 | NM_001009922 | Hs00295839_m1 | RCHY1 | ring finger and CHY zinc finger | 048N | PRD | N |
| | | | | | domain containing 1 | | | |
| 86 | 96 | NM_006405 | Hs00197392_m1 | TM9SF1 | transmembrane 9 superfamily | 048P | PRD | P |
| | | | | | member 1 | | | |
| 87 | 99 | NM_015633 | Hs00381867_m1 | FGFR1OP2 | FGFR1 oncogene partner 2 | 050N | PRD | N |
| 88 | 100 | NM_014865 | Hs00274505_m1 | NCAPD2 | non-SMC condensin I complex, | 050P | PRD | P |
| | | | | | subunit D2 | | | |
| 89 | 101 | NM_003268 | Hs00152825_m1 | TLR5 | toll-like receptor 5 | 051N | PRD | N |
| 90 | 102 | NM_016470 | Hs00212852_m1 | C20orf111 | chromosome 20 open reading | 051P | PRD | P |
| | | | | | frame 111 | | | |
| 91 | 103 | NM_172388 | Hs00542678_m1 | NFATC1 | nuclear factor of activated T- | 052N | PRD | N |
| | | | | | cells, cytoplasmic, calcineurin- | | | |
| | | | | | dependent 1 | | | |
| 92 | 104 | NM_024605 | Hs00226305_m1 | ARHGAP10 | Rho GTPase activating protein | 052P | PRD | P |
| | | | | | 10 | | | |
| 93 | 105 | NM_058192 | Hs00369703_m1 | RPUSD1 | RNA pseudouridylate synthase | 053N | PRD | N |
| | | | | | domain containing 1 | | | |
| 94 | 106 | NM_003400 | Hs00418963_m1 | XPO1 | exportin 1 (CRM1 homolog, | 053P | PRD | P |
| | | | | | yeast) | | | |
| 95 | 108 | NM_016447 | Hs00212785_m1 | MPP6 | membrane protein, palmitoylated | 054P | PRD | P |
| | | | | | 6 (MAGUK p55 subfamily | | | |
| | | | | | member 6) | | | |
| 96 | 109 | NM_004925 | Hs00185020_m1 | AQP3 | aquaporin 3 (Gill blood group) | 055N | PRD | N |
| 97 | 110 | NM_006348 | Hs00197140_m1 | COG5 | component of oligomeric golgi | 055P | PRD | P |
| | | | | | complex 5 | | | |
| 98 | 111 | NM_020320 | Hs00368084_m1 | RARS2 | arginyl-tRNA synthetase 2, | 056N | PRD | N |
| | | | | | mitochondrial | | | |
| 99 | 112 | NM_175617 | Hs01582977_gH | MT1E | metallothionein 1E | 056P | PRD | P |
| 100 | 113 | NM_018268 | Hs00217534_m1 | WDR41 | WD repeat domain 41 | 057N | PRD | N |
| 101 | 114 | NM_002882 | Hs01597912_g1 | RANBP1 | RAN binding protein 1 | 057P | PRD | P |
| 102 | 116 | NM_199367 | Hs00275795_m1 | SPG7 | spastic paraplegia 7 (pure and | 058P | PRD | P |
| | | | | | complicated autosomal | | | |
| | | | | | recessive) | | | |
| 103 | 117 | NM_006662 | Hs00198472_m1 | SRCAP | Snf2-related CREBBP activator | 059N | PRD | N |
| | | | | | protein | | | |
| 104 | 118 | NM_014254 | Hs00204546_m1 | TMEM5 | transmembrane protein 5 | 059P | PRD | P |
| 105 | 119 | NM_000355 | Hs00165902_m1 | TCN2 | transcobalamin II | 060N | PRD | N |
| 106 | 120 | NM_145799 | Hs00248408_m1 | SEPT6 | septin 6 | 060P | PRD | P |
| 107 | 121 | NM_013332 | Hs00203383_m1 | C7orf68 | chromosome 7 open reading | 061N | PRD | N |
| | | | | | frame 68 | | | |
| 108 | 122 | NM_014911 | Hs00208618_m1 | AAK1 | AP2 associated kinase 1 | 061P | PRD | P |
| 109 | 123 | NM_000067 | Hs00163869_m1 | CA2 | carbonic anhydrase II | 062N | PRD | N |
| 110 | 124 | NM_023080 | Hs00535769_m1 | C8orf33 | chromosome 8 open reading | 062P | PRD | P |
| | | | | | frame 33 | | | |
| 111 | 125 | NM_003473 | Hs00610137_m1 | STAM | signal transducing adaptor | 063N | PRD | N |
| | | | | | molecule (SH3 domain and | | | |
| | | | | | ITAM motif) 1 | | | |
| 112 | 126 | NM_022743 | Hs00224208_m1 | SMYD3 | SET and MYND domain | 063P | PRD | P |
| | | | | | containing 3 | | | |
| 113 | 127 | NM_003003 | Hs00608163_m1 | SEC14L1 | SEC14-like 1 (S. cerevisiae) | 064N | PRD | N |
| 114 | 128 | NM_000848 | Hs00265266_g1 | GSTM2 | glutathione S-transferase mu 2 | 064P | PRD | P |
| | | | | | (muscle) | | | |
| 115 | 130 | NM_003093 | Hs00853882_g1 | SNRPC | small nuclear ribonucleoprotein | 065P | PRD | P |
| | | | | | polypeptide C | | | |
| 116 | 131 | NM_021067 | Hs01040835_m1 | GINS1 | GINS complex subunit 1 (Psf1 | 066N | PRD | N |
| | | | | | homolog) | | | |
| 117 | 132 | NM_005184 | Hs00270914_m1 | CALM3 | calmodulin 3 (phosphorylase | 066P | PRD | P |
| | | | | | kinase, delta) | | | |
| 118 | 133 | NM_016310 | Hs00363121_m1 | POLR3K | polymerase (RNA) III (DNA | 067N | PRD | N |
| | | | | | directed) polypeptide K, 12.3 kDa | | | |
| 119 | 134 | NM_014901 | Hs00208576_m1 | RNF44 | ring finger protein 44 | 067P | PRD | P |
| 120 | 135 | NM_004255 | Hs00362067_m1 | COX5A | cytochrome c oxidase subunit Va | 068N | PRD | N |
| 121 | 136 | NM_032177 | Hs00536084_m1 | PHAX | phosphorylated adaptor for RNA | 068P | PRD | P |
| | | | | | export | | | |
| 122 | 137 | NM_020216 | Hs00220260_m1 | RNPEP | arginyl aminopeptidase | 069N | PRD | N |
| | | | | | (aminopeptidase B) | | | |
| 123 | 138 | NM_182922 | Hs00608563_m1 | HEATR3 | HEAT repeat containing 3 | 069P | PRD | P |
| 124 | 139 | NM_032412 | Hs00260900_m1 | C5orf32 | chromosome 5 open reading | 070N | PRD | N |
| | | | | | frame 32 | | | |
| 125 | 140 | NM_001707 | Hs00156055_m1 | BCL7B | B-cell CLL/lymphoma 7B | 070P | PRD | P |
| 126 | 141 | NM_006402 | Hs00246261_m1 | HBXIP | hepatitis B virus x interacting | 071N | PRD | N |
| | | | | | protein | | | |
| 127 | 142 | NM_139118 | Hs00217433_m1 | YY1AP1 | YY1 associated protein 1 | 071P | PRD | P |
| 128 | 143 | NM_006566 | Hs00170832_m1 | CD226 | CD226 molecule | 072N | PRD | N |
| 129 | 144 | NM_152320 | Hs01075391_m1 | ZNF641 | zinc finger protein 641 | 072P | PRD | P |
| 130 | 146 | NM_007249 | Hs00971557_m1 | KLF12 | Kruppel-like factor 12 | 073P | PRD | P |
| 131 | 147 | NM_024516 | Hs00225908_m1 | C16orf53 | chromosome 16 open reading | 074N | PRD | N |
| | | | | | frame 53 | | | |
| 132 | 148 | NM_015077 | Hs00248344_m1 | SARM1 | sterile alpha and TIR motif | 074P | PRD | P |
| | | | | | containing 1 | | | |
| 133 | 149 | NM_018177 | Hs00905983_m1 | N4BP2 | NEDD4 binding protein 2 | 075N | PRD | N |
| 134 | 150 | NM_001001660 | Hs01390827_g1 | LYRM5 | LYR motif containing 5 | 075P | PRD | P |
| 135 | 151 | NM_004169 | Hs00541038_m1 | SHMT1 | serine hydroxymethyltransferase | 076N | PRD | N |
| | | | | | 1 (soluble) | | | |
| 136 | 152 | NM_005951 | Hs00823168_g1 | MT1H | metallothionein 1H | 076P | PRD | P |
| 137 | 153 | NM_005234 | Hs00172870_m1 | NR2F6 | nuclear receptor subfamily 2, | 077N | PRD | N |
| | | | | | group F, member 6 | | | |
| 138 | 154 | NM_017761 | Hs02518187_g1 | PNRC2 | proline-rich nuclear receptor | 077P | PRD | P |
| | | | | | coactivator 2 | | | |
| 139 | 155 | NM_178009 | Hs00410739_m1 | DGKH | diacylglycerol kinase, eta | 078N | PRD | N |
| 140 | 156 | NM_014819 | Hs01122981_m1 | PJA2 | praja ring finger 2 | 078P | PRD | P |
| 141 | 157 | NM_001077191 | Hs01937849_s1 | GPBAR1 | G protein-coupled bile acid | 079N | PRD | N |
| | | | | | receptor 1 | | | |
| 142 | 158 | NM_015986 | Hs00367579_m1 | CRLF3 | cytokine receptor-like factor 3 | 079P | PRD | P |
| 143 | 159 | NM_012198 | Hs00201854_m1 | GCA | grancalcin, EF-hand calcium | 080N | PRD | N |
| | | | | | binding protein | | | |
| 144 | 160 | NM_002735 | Hs00406762_m1 | PRKAR1B | protein kinase, cAMP- | 080P | PRD | P |
| | | | | | dependent, regulatory, type I, | | | |
| | | | | | beta | | | |
| 145 | 161 | NM_032947 | Hs00383944_m1 | C5orf62 | chromosome 5 open reading | 081N | PRD | N |
| | | | | | frame 62 | | | |
| 146 | 162 | NM_005678 | Hs00243205_m1 | SNURF | SNRPN upstream reading | 081P | PRD | P |
| | | | | | frame, small nuclear | | | |
| | | | | | ribonucleoprotein polypeptide N | | | |
| 147 | 163 | NM_003956 | Hs02379634_s1 | CH25H | cholesterol 25-hydroxylase | 082N | PRD | N |
| 148 | 164 | NM_005950 | Hs02578922_gH | MT1G | metallothionein 1G | 082P | PRD | P |
| 149 | 165 | NM_003295 | Hs02621289_g1 | TPT1 | tumor protein, translationally- | 083N | PRD | N |
| | | | | | controlled 1 | | | |
| 150 | 166 | NM_001556 | Hs00233287_m1 | IKBKB | inhibitor of kappa light | 083P | PRD | P |
| | | | | | polypeptide gene enhancer in B- | | | |
| | | | | | cells, kinase beta | | | |
| 151 | 167 | NM_152889 | Hs00541730_m1 | CHST13 | carbohydrate (chondroitin 4) | 084N | PRD | N |
| | | | | | sulfotransferase 13 | | | |
| 152 | 168 | NM_001042588 | Hs00371639_m1 | SNUPN | snurportin 1 | 084P | PRD | P |
| 153 | 170 | NM_000981 | Hs02338565_gH | RPL19 | ribosomal protein L19 | 01HSK | HSK | — |
| 154 | 171 | NM_031369 | Hs01086912_m1 | HNRNPD | heterogeneous nuclear | 02HSK | HSK | — |
| | | | | | ribonucleoprotein D (AU-rich | | | |
| | | | | | element RNA binding protein 1, | | | |
| | | | | | 37 kDa) | | | |
| 155 | 172 | NM_001023 | Hs00828752_gH | RPS20 | ribosomal protein S20 | 03HSK | HSK | — |
| 156 | 173 | NM_016093 | Hs01631495_s1 | RPL26L1 | ribosomal protein L26-like 1 | 04HSK | HSK | — |
| 157 | 174 | NM_022170 | Hs00254535_m1 | EIF4H | eukaryotic translation initiation | 05HSK | HSK | — |
| | | | | | factor 4H | | | |
| 158 | 175 | NM_032195 | Hs00371372_m1 | SON | SON DNA binding protein | 06HSK | HSK | — |
| 159 | 176 | NM_016061 | Hs00763191_s1 | YPEL5 | yippee-like 5 (Drosophila) | 07HSK | HSK | — |
| 160 | 177 | NM_013379 | Hs01115161_m1 | DPP7 | dipeptidyl-peptidase 7 | 08HSK | HSK | — |
| 161 | 178 | NM_004034 | Hs00559413_m1 | ANXA7 | annexin A7 | 10HSK | HSK | — |
| 162 | 179 | NM_001033112 | Hs00212868_m1 | PAIP2 | poly(A) binding protein | 11HSK | HSK | — |
| | | | | | interacting protein 2 | | | |
| 163 | 180 | NM_006861 | Hs00199284_m1 | RAB35 | RAB35, member RAS oncogene | 12HSK | HSK | — |
| | | | | | family | | | |
| 164 | 181 | NM_007065 | Hs00606477_m1 | CDC37 | cell division cycle 37 homolog | 13HSK | HSK | — |
| | | | | | (S. cerevisiae) | | | |
| 165 | 182 | NM_005626 | Hs00194538_m1 | SRSF4 | serine/arginine-rich splicing | 14HSK | HSK | — |
| | | | | | factor 4 | | | |
| 166 | 183 | NM_018064 | Hs00363236_m1 | AKIRIN2 | akirin 2 | 15HSK | HSK | — |
| 167 | 184 | NM_030818 | Hs00229388_m1 | CCDC130 | coiled-coil domain containing | 16HSK | HSK | — |
| | | | | | 130 | | | |
| 168 | 185 | NM_006110 | Hs00272036_m1 | CD2BP2 | CD2 (cytoplasmic tail) binding | 17HSK | HSK | — |
| | | | | | protein 2 | | | |
| 169 | 186 | NM_006327 | Hs00197056_m1 | TIMM23 | translocase of inner | 18HSK | HSK | — |
| | | | | | mitochondrial membrane 23 | | | |
| | | | | | homolog (yeast), translocase of | | | |
| | | | | | inner mitochondrial membrane | | | |
| | | | | | 23 homolog B (yeast) | | | |
| 170 | 187 | NM_005466 | Hs00193824_m1 | MED6 | mediator complex subunit 6 | 20HSK | HSK | — |
| 171 | 188 | NM_006600 | Hs00702452_s1 | NUDC | nuclear distribution gene C | 21HSK | HSK | — |
| | | | | | homolog (A. nidulans) | | | |
| 172 | 189 | NM_020141 | Hs00220038_m1 | TMEM167B | transmembrane protein 167B | 22HSK | HSK | — |
| 173 | 190 | NM_030914 | Hs00229455_m1 | URM1 | ubiquitin related modifier 1 | 23HSK | HSK | — |
| 174 | 191 | NM_014607 | Hs00412682_m1 | UBXN4 | UBX domain protein 4 | 24HSK | HSK | — |
| 175 | 192 | NM_173607 | Hs00380814_m1 | FAM177A1 | family with sequence similarity | 25HSK | HSK | — |
| | | | | | 177, member A1 | | | |

### Example 12

This example includes a description of evaluation of RGP (ratiometric gene pair) candidates for GVHD prediction.

Introduction:

Ratiometric gene pairs (RGPs) provide for additional outcome predictive robustness through (1) self-calibration by dividing-out background variation, and (2) capturing potential competitive pathway interaction effects between genes at the expression level. RGPs are determined by dividing the expression level of a select single gene by the expression level of another select single gene.

Determination of RGPs:

In PBDs technical implementation, because the RRCF data is expressed in logarithmic form of mRNA concentration measurement levels (see above), i.e., RRCF X˜log(gene X) and RL2F X˜log(gene X), the ratio of gene X/gene Y expression, in logarithmic form log(gene X/gene Y), can also be expressed as the difference log(gene X)−log(gene Y), which is equivalent to RRCF X˜RRCF Y. Therefore, in all usage below, RGP values for RRCF data are defined as follows:

RGP=RRCF X−RRCF Y.

Note that RGP values can also be directly calculated from the RL2F data, before background subtraction of HSK genes, because the HSK background subtraction itself is subtracted out in the RGP calculation. Given that RRCF=RL2F−RHSKAG6 (see above), then RGP=(RL2F X−RHSKAG6)−(RL2F Y−RHSKAG6), therefore, alternatively:

RGP=RL2FX−RL2FY.

The RGP values for all 180 samples were determined for the complete set 15,225 unique RGPs from the RRCF data of all select 175 SGs (single genes) that passed QC (as described above). For 175 SGs (single genes), the total number of RGPs, i.e. unique pair-wise SG combinations, is defined as (175²−175)/2=15,225.

Determining Outcome Predictive Performance of RGPs:

GVHD outcome predictive performance was evaluated for each of the 15,225 RGPs by determining class separation:

(1) p-values using the 2-tailed, heteroscedastic T-test, and

(2) accuracies (ACC) using LDA (linear discriminant analysis).

T-test p-value and LDA accuracy calculations were carried out as described above.

The 180 sample dataset comprises the following four sample classes:
(1) Gneg: 59 samples for which no form of acute or chronic GVHD was observed in the transplant recipients
(2) Gpos: 121 samples for which any form of acute of chronic GVHD was observed in the transplant recipients
(3) Gag2: 110 samples for which acute grade II, III or IV GVHD, either with or without chronic GVHD, was observed in the transplant recipients
(4) Gag3: 77 samples for which severe acute grade III or IV GVHD, either with or without chronic GVHD, was observed in the transplant recipients

Assuming a prevalence, P, (overall occurrence in transplantations) of acute grade II, III or IV GVHD (Gag2) in the commonly accepted range of 35% to 55%, with a midpoint of 45%, the 110 Gag2 cases would be expected to be observed in a total of 110/0.45≈244 transplantations. The fraction of the 77 acute grade III or IV GVHD (Gag3) from such a total of ˜244 transplantations then corresponds to 0.315, i.e. 31.5%, which is within the commonly accepted prevalence range of acute grade III or IV GVHD. Per the definition, all of the 77 Gag3 cases are part of the 110 Gag2 cases.

In summary, the proportion of Gag3 cases within the Gag2 cases is largely consistent with Gag2 prevalences in the range of 35% to 55%, and Gag3 prevalences in the range of 15% to 35%. Therefore, projections of potential GVHD reductions for Gag2 and Gag3 outcomes, when using the GVHD outcome prediction to restrict donors to the ones predicted by the analysis to not cause GVHD, would be based on predictive models that are trained using well-balanced proportions of Gag2 and Gag3 samples, representative of commonly accepted ranges of Gag2 and Gag3 prevalences.

GVHD outcome predictive performance (T-test and LDA) was determined for the following class divisions:

(1) Gneg vs. Gpos

(2) Gneg vs. Gag2

(3) Gneg vs. Gag3

Note for all LDA calculation for each of the 3 different class divisions, the LDA separatrix from the Gneg vs. Gpos division was used, determined as the midpoint between the average RGP value of the 59 Gneg samples, and the average RGP value of the 121 Gpos samples.

Note that the accuracies were determined using “balanced” proportional representations of negatively and positively classified samples, based on imposing a balanced prevalence of P_{b}=0.5 (50%) of GVHD positive cases. Note that all 4 confusion matrix classification values (CMCVs), TN, FP, TP, FN, are represented as proportions of a total of 1, i.e. all 4 values must always add up to 1. Balanced CMCVs (noted by subscript “b”), are determined from initial CMCVs (noted by subscript “0”) based on an initial prevalence Pₒ, according to the following equations:

TN_{b}=(1−P_{b})/(1−P₀)*TN₀ (1)

FP_{b}=(1−P_{b})/(1−P₀)*FP₀ (2)

TP_{b}=P_{b}/P₀*TP₀ (3)

FN_{b}=P_{b}/P₀*FN₀ (4)

ACC, accuracy, adjusted for balanced prevalence, P_{b}, is defined as follows (also see P1 and P2):

ACC=(TN_{b}+TP_{b})/(TN_{b}+FN_{b}+TP_{b}+FP_{b})

Evaluation of Outcome Predictive Performance of RGPs:

The class discrimination analysis provides an output of 6 performance variables, i.e. p-values and accuracies for each of the 3 divisions, for all 15,225 RGPs (see table RGP348). The 6 outcome predictive performance variables were ranked from 1 to 15,225, respectively
(1) according to best performing (minimal) to worst performing (maximal) p-values for each of the 3 divisions; and
(2) according to best performing (maximal) to worst performing (minimal) outcome predictive LDA accuracies for each of the 3 divisions.

As an initial reduction of candidate RGPs for further refinement into a GVHD outcome prediction profile, a set of 348 RGPs (RGP348; see Table 14) was selected by requiring each RGP to have over all 6 predictive performance variable ranks a maximal rank (from 1 to 15,225)<=2000, and minimal rank<=200. In other words, all 6 outcome predictive performance variables had to be among the top 2000 (13.1%), and at least 1 of the 6 outcome predictive performance variables had to be among the top 200 (1.3%). Within the RGP348 list (Table 14), 128 of the 175 SGs are represented, ranging from participation of each SG in 1 to 53 different RGPs (see Table 15, SG128).

TABLE 14
List of 348 RGPs (RGP348) with high outcome predictive performance ranks

| | Gneg vs. | Gneg vs. | Gneg vs. | ACC | ACC | | Rank Gneg vs. | Rank Gneg vs. | Rank Gneg vs. | Rank | Rank | Rank | | | |
|---|---|---|---|---|---|---|---|---|---|---|---|---|---|---|---|
| RGP | Gpos | Gag2 | Gag3 | Gneg | Gneg | ACC | Gpos | Gag2 | Gag3 | ACC | ACC | ACC | Min rank | Max rank | Median rank |
| ABI Gene | T-test | T-test | T-test | vs. | vs. | Gneg vs. | T-test | T-test | T-test | Gneg vs. | Gneg vs. | Gneg vs. | p-value and | p-value and | p-value and |
| Symbol | p-value | p-value | p-value | Gpos | Gag2 | Gag3 | p-value | p-value | p-value | Gpos | Gag2 | Gag3 | ACC | ACC | ACC |
| VAMP2-SEC14L1 | 4.8E−04 | 2.1E−04 | 3.1E−05 | 0.68 | 0.69 | 0.69 | 118 | 72 | 8 | 1 | 1 | 4 | 1 | 118 | 6 |
| TMEM49-TATDN1 | 9.0E−06 | 9.3E−06 | 1.5E−06 | 0.64 | 0.64 | 0.68 | 2 | 3 | 1 | 112.5 | 90.5 | 15.5 | 1 | 112.5 | 9.25 |
| AEBP1-SEC14L1 | 5.4E−04 | 1.0E−03 | 9.1E−05 | 0.67 | 0.67 | 0.70 | 127 | 292 | 26 | 3 | 9 | 1 | 1 | 292 | 17.5 |
| VAMP2-CRTAP | 1.4E−04 | 9.1E−05 | 3.9E−05 | 0.66 | 0.66 | 0.66 | 36 | 27 | 13 | 17.5 | 18 | 60 | 13 | 60 | 22.5 |
| GINS1-MT1H | 1.2E−04 | 7.3E−05 | 2.5E−03 | 0.65 | 0.66 | 0.65 | 28 | 20 | 470 | 21 | 22 | 124.5 | 20 | 470 | 25 |
| VAMP2-LAPTM5 | 1.9E−04 | 1.7E−04 | 8.9E−05 | 0.66 | 0.66 | 0.67 | 43 | 48 | 25 | 12.5 | 11 | 26.5 | 11 | 48 | 25.75 |
| MT1E-GINS1 | 2.5E−05 | 9.4E−06 | 2.6E−04 | 0.65 | 0.66 | 0.65 | 7 | 4 | 73 | 34 | 20.5 | 145 | 4 | 145 | 27.25 |
| C20orf111-SEC14L1 | 1.3E−03 | 8.3E−04 | 2.1E−04 | 0.67 | 0.67 | 0.68 | 298 | 248 | 61 | 5 | 6 | 12 | 5 | 298 | 36.5 |
| AEBP1-RPUSD1 | 3.9E−05 | 1.8E−04 | 1.6E−04 | 0.66 | 0.65 | 0.66 | 13 | 52 | 40 | 12.5 | 39 | 72 | 12.5 | 72 | 39.5 |
| AEBP1-NCDN | 1.4E−04 | 3.7E−04 | 2.2E−04 | 0.65 | 0.65 | 0.67 | 33 | 130 | 63 | 25 | 45.5 | 37.5 | 25 | 130 | 41.5 |
| VAMP2-NCOA4 | 5.6E−05 | 2.7E−05 | 1.3E−05 | 0.64 | 0.65 | 0.66 | 16 | 9 | 5 | 74.5 | 67.5 | 72 | 5 | 74.5 | 41.75 |
| ABHD12-MPP6 | 1.3E−03 | 2.0E−03 | 3.5E−05 | 0.65 | 0.65 | 0.69 | 289 | 458 | 12 | 34 | 52.5 | 2 | 2 | 458 | 43.25 |
| ANAPC11-GINS1 | 3.8E−05 | 1.7E−05 | 1.7E−04 | 0.64 | 0.64 | 0.65 | 12 | 6 | 47 | 44 | 81.5 | 124.5 | 6 | 124.5 | 45.5 |
| VAMP2-ZFAND5 | 3.1E−04 | 9.9E−05 | 3.1E−05 | 0.62 | 0.64 | 0.67 | 68 | 28 | 9 | 215.5 | 77.5 | 17.5 | 9 | 215.5 | 48 |
| VAMP2-C5orf32 | 1.1E−04 | 1.3E−04 | 1.8E−04 | 0.64 | 0.65 | 0.66 | 25 | 37 | 52 | 44 | 58 | 80 | 25 | 80 | 48 |
| NCOA4-C8orf33 | 4.6E−04 | 3.1E−04 | 7.8E−05 | 0.64 | 0.65 | 0.66 | 111 | 107 | 21 | 55.5 | 39 | 44 | 21 | 111 | 49.75 |
| VAMP2-GCH1 | 1.5E−04 | 2.3E−04 | 2.0E−04 | 0.66 | 0.65 | 0.64 | 38 | 82 | 57 | 17.5 | 45.5 | 222.5 | 17.5 | 222.5 | 51.25 |
| GINS1-PRKAR1B | 1.7E−04 | 1.8E−04 | 2.0E−04 | 0.65 | 0.65 | 0.66 | 40 | 56 | 56 | 34 | 52.5 | 94 | 34 | 94 | 54.25 |
| VAMP2-C5orf62 | 2.2E−03 | 1.5E−03 | 2.1E−04 | 0.65 | 0.65 | 0.66 | 445 | 381 | 60 | 38 | 45.5 | 60 | 38 | 445 | 60 |
| VAMP2-KIAA1949 | 1.4E−04 | 1.8E−04 | 7.8E−05 | 0.64 | 0.64 | 0.66 | 34 | 55 | 20 | 74.5 | 98 | 72 | 20 | 98 | 63.5 |
| MPP6-SEC14L1 | 5.0E−03 | 4.0E−03 | 1.7E−04 | 0.64 | 0.66 | 0.67 | 768 | 739 | 45 | 82.5 | 24 | 20 | 20 | 768 | 63.75 |
| SELM-RPUSD1 | 3.9E−04 | 2.9E−04 | 1.5E−03 | 0.65 | 0.65 | 0.67 | 94 | 102 | 312 | 30 | 35 | 29 | 29 | 312 | 64.5 |
| TMEM49-PLAC8 | 2.8E−05 | 8.7E−05 | 2.7E−05 | 0.64 | 0.63 | 0.65 | 8 | 24 | 7 | 112.5 | 238.5 | 133 | 7 | 238.5 | 68.25 |
| TMEM49-FLT3LG | 3.0E−03 | 2.7E−03 | 2.8E−04 | 0.64 | 0.65 | 0.66 | 539 | 564 | 80 | 44 | 58 | 47 | 44 | 564 | 69 |
| MPP5-SEC14L1 | 4.4E−03 | 3.0E−03 | 2.7E−04 | 0.64 | 0.65 | 0.69 | 699 | 602 | 78 | 68 | 45.5 | 8.5 | 8.5 | 699 | 73 |
| SEC14L1-LYRM5 | 3.0E−03 | 2.0E−03 | 3.5E−04 | 0.64 | 0.65 | 0.67 | 543 | 469 | 94 | 52.5 | 27 | 37.5 | 27 | 543 | 73.25 |
| PDCD6IP-LYRM5 | 2.6E−03 | 4.1E−03 | 4.9E−04 | 0.66 | 0.66 | 0.68 | 503 | 746 | 126 | 8.5 | 23 | 13 | 8.5 | 746 | 74.5 |
| NCOA4-PLAC8 | 1.0E−03 | 7.5E−04 | 1.1E−04 | 0.64 | 0.64 | 0.66 | 235 | 239 | 31 | 58.5 | 90.5 | 50 | 31 | 239 | 74.5 |
| TMEM49-MRPL42 | 1.9E−04 | 3.4E−04 | 4.8E−04 | 0.65 | 0.65 | 0.65 | 45 | 120 | 125 | 25 | 45.5 | 107 | 25 | 125 | 76.25 |
| PDCD6IP-TATDN1 | 2.7E−03 | 1.8E−03 | 3.4E−04 | 0.64 | 0.64 | 0.66 | 516 | 440 | 91 | 68 | 72.5 | 60 | 60 | 516 | 81.75 |
| TMEM49-VAMP2 | 2.4E−06 | 3.9E−06 | 9.7E−06 | 0.63 | 0.63 | 0.62 | 1 | 1 | 4 | 164 | 285 | 570.5 | 1 | 570.5 | 84 |
| MAF-RPS20 | 2.0E−05 | 2.0E−05 | 1.6E−04 | 0.63 | 0.64 | 0.62 | 5 | 7 | 41 | 181.5 | 138 | 683.5 | 5 | 683.5 | 89.5 |
| SEC14L1-RPL19 | 6.2E−03 | 2.7E−03 | 5.9E−04 | 0.65 | 0.67 | 0.67 | 866 | 573 | 149 | 22.5 | 10 | 32 | 10 | 866 | 90.5 |
| ADRB2-MT1E | 8.9E−05 | 4.8E−05 | 4.5E−04 | 0.63 | 0.65 | 0.64 | 22 | 15 | 119 | 139 | 67.5 | 180 | 15 | 180 | 93.25 |
| NCOA4-PAIP2 | 3.7E−04 | 2.1E−04 | 3.4E−04 | 0.63 | 0.64 | 0.65 | 89 | 71 | 90 | 139 | 98 | 119 | 71 | 139 | 94 |
| NCOA4-MPP6 | 2.5E−04 | 2.6E−04 | 4.6E−06 | 0.63 | 0.63 | 0.65 | 54 | 90 | 3 | 145 | 175 | 100 | 3 | 175 | 95 |
| CRTAP-LYRM5 | 2.0E−03 | 2.1E−03 | 7.3E−04 | 0.66 | 0.66 | 0.67 | 425 | 490 | 177 | 11 | 12 | 21.5 | 11 | 490 | 99.25 |
| MRPL42-GINS1 | 7.5E−05 | 4.5E−05 | 2.7E−04 | 0.63 | 0.63 | 0.64 | 19 | 14 | 77 | 123 | 165.5 | 262 | 14 | 262 | 100 |
| AAK1-SEC14L1 | 1.9E−02 | 1.3E−02 | 5.4E−04 | 0.64 | 0.65 | 0.69 | 1789 | 1549 | 138 | 64 | 31.5 | 5.5 | 5.5 | 1789 | 101 |
| SELM-NCOA4 | 8.9E−04 | 3.4E−04 | 1.1E−04 | 0.62 | 0.64 | 0.66 | 211 | 117 | 30 | 261.5 | 90.5 | 88 | 30 | 261.5 | 103.75 |
| PREX1-KLF12 | 1.2E−03 | 9.2E−04 | 2.6E−04 | 0.64 | 0.64 | 0.65 | 268 | 266 | 75 | 103.5 | 104.5 | 107 | 75 | 268 | 105.75 |
| CRTAP-PLAC8 | 3.7E−03 | 3.6E−03 | 6.0E−04 | 0.65 | 0.65 | 0.67 | 616 | 691 | 150 | 27 | 67.5 | 26.5 | 26.5 | 691 | 108.75 |
| RPL37-GINS1 | 5.0E−04 | 2.8E−04 | 1.3E−03 | 0.64 | 0.64 | 0.64 | 119 | 100 | 288 | 89 | 85 | 229 | 85 | 288 | 109.5 |
| XRCC1-PREX1 | 1.1E−03 | 1.5E−03 | 5.3E−04 | 0.64 | 0.64 | 0.67 | 244 | 371 | 135 | 42 | 85 | 39.5 | 39.5 | 371 | 110 |
| SNX27-TATDN1 | 8.5E−04 | 7.5E−04 | 2.9E−04 | 0.64 | 0.64 | 0.66 | 205 | 240 | 82 | 74.5 | 138 | 72 | 72 | 240 | 110 |
| AEBP1-TM9SF1 | 3.4E−04 | 9.6E−04 | 3.7E−04 | 0.64 | 0.64 | 0.65 | 78 | 281 | 99 | 58.5 | 129.5 | 133 | 58.5 | 281 | 114.25 |
| SEC14L1-CALM3 | 6.5E−03 | 5.0E−03 | 8.3E−04 | 0.64 | 0.65 | 0.68 | 898 | 858 | 197 | 48.5 | 31.5 | 11 | 11 | 898 | 122.75 |
| TMEM49-ANXA11 | 3.2E−04 | 1.1E−03 | 1.3E−03 | 0.64 | 0.64 | 0.65 | 73 | 324 | 285 | 55.5 | 138 | 119 | 55.5 | 324 | 128.5 |
| C5orf62-RPS20 | 6.0E−03 | 4.4E−03 | 6.7E−04 | 0.64 | 0.64 | 0.66 | 846 | 787 | 163 | 74.5 | 98 | 72 | 72 | 846 | 130.5 |
| HMGB2-SEC14L1 | 3.5E−03 | 2.3E−03 | 1.1E−03 | 0.67 | 0.68 | 0.68 | 597 | 512 | 250 | 2 | 2.5 | 14 | 2 | 597 | 132 |
| ADRB2-AEBP1 | 1.3E−03 | 3.4E−03 | 6.2E−04 | 0.64 | 0.64 | 0.67 | 300 | 670 | 158 | 48.5 | 111.5 | 32 | 32 | 670 | 134.75 |
| VAMP2-MAF | 4.2E−04 | 3.2E−04 | 1.1E−03 | 0.63 | 0.64 | 0.63 | 102 | 112 | 242 | 142.5 | 129.5 | 389.5 | 102 | 389.5 | 136 |
| XRCC1-SEC14L1 | 8.8E−03 | 7.6E−03 | 1.0E−03 | 0.65 | 0.65 | 0.69 | 1127 | 1120 | 239 | 34 | 31.5 | 5.5 | 5.5 | 1127 | 136.5 |
| ABHD12-MRPL42 | 4.0E−03 | 3.4E−03 | 8.2E−04 | 0.64 | 0.65 | 0.66 | 661 | 660 | 193 | 80.5 | 64.5 | 50 | 50 | 661 | 136.75 |
| AEBP1-SARM1 | 7.8E−04 | 1.6E−03 | 1.5E−03 | 0.66 | 0.66 | 0.66 | 186 | 404 | 317 | 7 | 14 | 88 | 7 | 404 | 137 |
| TMEM49-AEBP1 | 8.5E−05 | 4.8E−04 | 1.4E−04 | 0.64 | 0.62 | 0.62 | 20 | 156 | 39 | 118 | 406 | 655.5 | 20 | 655.5 | 137 |
| MAF-LYRM5 | 2.4E−04 | 3.7E−04 | 1.2E−03 | 0.64 | 0.64 | 0.63 | 52 | 129 | 271 | 103.5 | 147.5 | 449.5 | 52 | 449.5 | 138.25 |
| VAMP2-PREX1 | 1.4E−04 | 9.1E−05 | 6.0E−05 | 0.62 | 0.63 | 0.63 | 32 | 25 | 16 | 245.5 | 253 | 349 | 16 | 349 | 138.75 |
| NDUFC1-SEC14L1 | 1.3E−02 | 8.2E−03 | 1.2E−03 | 0.67 | 0.67 | 0.69 | 1438 | 1180 | 274 | 5 | 6 | 8.5 | 5 | 1438 | 141.25 |
| C5orf62-RPL19 | 7.7E−03 | 5.1E−03 | 5.8E−04 | 0.63 | 0.64 | 0.66 | 1007 | 865 | 146 | 139 | 138 | 72 | 72 | 1007 | 142.5 |
| SELM-CRTAP | 3.3E−03 | 1.8E−03 | 6.0E−04 | 0.64 | 0.64 | 0.65 | 572 | 438 | 153 | 112.5 | 90.5 | 133 | 90.5 | 572 | 143 |
| AEBP1-ZFAND5 | 6.5E−04 | 1.5E−03 | 1.3E−04 | 0.63 | 0.63 | 0.65 | 148 | 380 | 37 | 145 | 226.5 | 100 | 37 | 380 | 146.5 |
| NPTN-AEBP1 | 7.9E−04 | 2.0E−03 | 1.6E−03 | 0.67 | 0.65 | 0.65 | 189 | 464 | 327 | 5 | 27 | 107 | 5 | 464 | 148 |
| C8orf33-SEC14L1 | 7.0E−03 | 4.6E−03 | 7.6E−04 | 0.64 | 0.65 | 0.69 | 942 | 812 | 184 | 116.5 | 36 | 7 | 7 | 942 | 150.25 |
| CRTAP-CALM3 | 1.4E−03 | 1.8E−03 | 5.1E−04 | 0.64 | 0.64 | 0.65 | 320 | 433 | 131 | 98 | 155.5 | 145 | 98 | 433 | 150.25 |
| VAMP2-FOXN2 | 1.3E−03 | 8.7E−04 | 7.0E−04 | 0.63 | 0.64 | 0.65 | 295 | 255 | 169 | 134.5 | 104.5 | 107 | 104.5 | 295 | 151.75 |
| SEC14L1-SON | 7.3E−03 | 4.3E−03 | 1.1E−03 | 0.65 | 0.66 | 0.66 | 977 | 780 | 244 | 38 | 18 | 60 | 18 | 977 | 152 |
| SEC14L1-RPS20 | 5.4E−03 | 3.0E−03 | 8.9E−04 | 0.64 | 0.65 | 0.65 | 811 | 608 | 204 | 103.5 | 45.5 | 107 | 45.5 | 811 | 155.5 |
| PREX1-C20orf111 | 3.1E−04 | 3.3E−04 | 2.9E−04 | 0.63 | 0.62 | 0.63 | 70 | 116 | 81 | 195.5 | 316.5 | 302 | 70 | 316.5 | 155.75 |
| NR2F6-PRKAR1B | 3.3E−03 | 5.5E−03 | 1.0E−03 | 0.65 | 0.65 | 0.66 | 580 | 910 | 236 | 30 | 58 | 80 | 30 | 910 | 158 |
| TMEM8B-C5orf62 | 1.7E−03 | 1.0E−03 | 1.7E−04 | 0.63 | 0.64 | 0.68 | 362 | 295 | 44 | 189.5 | 129.5 | 15.5 | 15.5 | 362 | 159.5 |
| VAMP2-TMEM5 | 8.6E−04 | 1.0E−03 | 1.6E−03 | 0.64 | 0.64 | 0.66 | 208 | 293 | 326 | 64 | 111.5 | 53.5 | 53.5 | 326 | 159.75 |
| AP2A1-MPP6 | 6.3E−03 | 8.2E−03 | 8.2E−04 | 0.64 | 0.64 | 0.67 | 882 | 1179 | 192 | 80.5 | 129.5 | 30 | 30 | 1179 | 160.75 |
| VAMP2-ABHD12 | 1.5E−05 | 1.3E−05 | 1.6E−06 | 0.61 | 0.62 | 0.63 | 3 | 5 | 2 | 579 | 324 | 389.5 | 2 | 579 | 164.5 |
| MT1E-SPG7 | 7.4E−04 | 2.8E−04 | 3.9E−03 | 0.63 | 0.64 | 0.62 | 174 | 98 | 644 | 158 | 118.5 | 517 | 98 | 644 | 166 |
| FLT3LG-GINS1 | 5.2E−04 | 2.3E−04 | 3.8E−04 | 0.62 | 0.63 | 0.62 | 123 | 83 | 101 | 285.5 | 209.5 | 517 | 83 | 517 | 166.25 |
| VAMP2-URM1 | 1.9E−05 | 6.0E−06 | 3.4E−05 | 0.62 | 0.62 | 0.61 | 4 | 2 | 10 | 435 | 324 | 1027.5 | 2 | 1027.5 | 167 |
| VAMP2-TMEM167B | 6.1E−03 | 2.4E−03 | 7.3E−04 | 0.63 | 0.64 | 0.66 | 860 | 520 | 176 | 158 | 81.5 | 80 | 80 | 860 | 167 |
| SEC14L1-SNURF | 9.3E−03 | 6.4E−03 | 1.3E−03 | 0.64 | 0.66 | 0.67 | 1171 | 1014 | 283 | 55.5 | 15 | 26.5 | 15 | 1171 | 169.25 |
| FOXN2-RPS20 | 2.1E−03 | 1.5E−03 | 9.5E−04 | 0.63 | 0.65 | 0.66 | 430 | 387 | 218 | 123 | 58 | 80 | 58 | 430 | 170.5 |
| SEPT6-GINS1 | 1.6E−04 | 1.6E−04 | 4.5E−04 | 0.62 | 0.63 | 0.63 | 39 | 45 | 118 | 229 | 268.5 | 449.5 | 39 | 449.5 | 173.5 |
| GINS1-RPL19 | 3.1E−04 | 1.7E−04 | 8.3E−04 | 0.63 | 0.63 | 0.62 | 69 | 50 | 196 | 158 | 209.5 | 517 | 50 | 517 | 177 |
| PLAC8-SEC14L1 | 1.4E−02 | 1.0E−02 | 1.6E−03 | 0.66 | 0.67 | 0.69 | 1508 | 1329 | 339 | 17.5 | 6 | 3 | 3 | 1508 | 178.25 |
| PPHLN1-SEC14L1 | 8.5E−03 | 7.5E−03 | 1.3E−03 | 0.64 | 0.65 | 0.66 | 1090 | 1113 | 282 | 74.5 | 25 | 44 | 25 | 1113 | 178.25 |
| TATDN1-SEC14L1 | 1.0E−02 | 5.1E−03 | 7.8E−04 | 0.63 | 0.64 | 0.66 | 1236 | 862 | 187 | 181.5 | 138 | 72 | 72 | 1236 | 184.25 |
| TMEM49-LYRM5 | 3.2E−05 | 9.1E−05 | 6.8E−05 | 0.62 | 0.62 | 0.63 | 10 | 26 | 19 | 344 | 422.5 | 434 | 10 | 434 | 185 |
| PDCD6IP-MT1E | 4.3E−04 | 1.9E−04 | 4.5E−03 | 0.62 | 0.63 | 0.61 | 104 | 58 | 703 | 207 | 165.5 | 958 | 58 | 958 | 186.25 |
| AEBP1-GRK6 | 9.6E−04 | 2.8E−03 | 6.3E−04 | 0.64 | 0.63 | 0.65 | 222 | 586 | 159 | 84 | 216.5 | 114 | 84 | 586 | 187.75 |
| CRTAP-MPP6 | 3.8E−03 | 4.8E−03 | 2.9E−04 | 0.63 | 0.63 | 0.65 | 646 | 833 | 84 | 145 | 226.5 | 154.5 | 84 | 833 | 190.5 |
| AEBP1-SNX27 | 1.3E−04 | 5.8E−04 | 1.7E−04 | 0.63 | 0.61 | 0.63 | 30 | 193 | 51 | 195.5 | 571 | 302 | 30 | 571 | 194.25 |
| VAMP2-GINS1 | 3.7E−05 | 2.1E−05 | 1.3E−04 | 0.62 | 0.62 | 0.61 | 11 | 8 | 36 | 402 | 355.5 | 861 | 8 | 861 | 195.75 |
| PREX1-MPP6 | 8.4E−04 | 1.0E−03 | 6.7E−05 | 0.63 | 0.63 | 0.66 | 203 | 307 | 18 | 189.5 | 238.5 | 88 | 18 | 307 | 196.25 |
| TATDN1-CCDC6 | 1.5E−02 | 6.4E−03 | 8.4E−04 | 0.63 | 0.63 | 0.67 | 1607 | 1026 | 199 | 199.5 | 173 | 23.5 | 23.5 | 1607 | 199.25 |
| NCOA4-SNURF | 4.7E−03 | 3.9E−03 | 1.9E−03 | 0.66 | 0.68 | 0.68 | 728 | 719 | 391 | 10 | 2.5 | 10 | 2.5 | 728 | 200.5 |
| SEC14L1-PAIP2 | 5.5E−03 | 2.8E−03 | 1.1E−03 | 0.63 | 0.64 | 0.65 | 814 | 584 | 261 | 129.5 | 77.5 | 145 | 77.5 | 814 | 203 |
| TMEM8B-TM9SF1 | 2.2E−03 | 1.2E−03 | 1.1E−03 | 0.63 | 0.64 | 0.65 | 463 | 337 | 259 | 149 | 121 | 128 | 121 | 463 | 204 |
| PREX1-SNURF | 6.4E−04 | 5.8E−04 | 3.2E−04 | 0.62 | 0.63 | 0.64 | 146 | 194 | 87 | 344 | 226.5 | 215 | 87 | 344 | 204.5 |
| AEBP1-NCOA4 | 2.6E−04 | 6.5E−04 | 1.2E−04 | 0.63 | 0.62 | 0.64 | 55 | 211 | 35 | 199.5 | 307 | 235 | 35 | 307 | 205.25 |
| VAMP2-SERPINB9 | 1.8E−03 | 1.1E−03 | 1.2E−03 | 0.63 | 0.64 | 0.65 | 388 | 316 | 269 | 129.5 | 111.5 | 145 | 111.5 | 388 | 207 |
| SELM-GINS1 | 9.8E−05 | 4.4E−05 | 2.1E−04 | 0.61 | 0.62 | 0.62 | 23 | 13 | 59 | 527 | 355.5 | 623.5 | 13 | 623.5 | 207.25 |
| GINS1-MT1G | 4.7E−04 | 2.4E−04 | 7.4E−03 | 0.62 | 0.63 | 0.61 | 114 | 86 | 1047 | 215.5 | 200.5 | 1068 | 86 | 1068 | 208 |
| AEBP1-SPG7 | 1.2E−04 | 2.2E−04 | 9.1E−05 | 0.62 | 0.61 | 0.61 | 29 | 74 | 28 | 344 | 571 | 834.5 | 28 | 834.5 | 209 |
| SEC14L1-KLF12 | 1.2E−02 | 7.3E−03 | 1.0E−03 | 0.63 | 0.64 | 0.66 | 1361 | 1091 | 231 | 189.5 | 90.5 | 50 | 50 | 1361 | 210.25 |
| SEC14L1-PRKAR1B | 7.7E−03 | 8.5E−03 | 7.5E−04 | 0.63 | 0.63 | 0.67 | 1009 | 1204 | 180 | 195.5 | 226.5 | 35.5 | 35.5 | 1204 | 211 |
| SMNDC1-SEC14L1 | 6.4E−03 | 4.5E−03 | 1.1E−03 | 0.63 | 0.64 | 0.65 | 894 | 801 | 251 | 172 | 147.5 | 162.5 | 147.5 | 894 | 211.5 |
| VAMP2-RARS2 | 4.3E−03 | 1.6E−03 | 1.5E−03 | 0.64 | 0.64 | 0.66 | 686 | 409 | 320 | 103.5 | 72.5 | 60 | 60 | 686 | 211.75 |
| STRADB-GINS1 | 3.4E−04 | 2.3E−04 | 4.5E−04 | 0.62 | 0.62 | 0.61 | 79 | 84 | 117 | 308.5 | 355.5 | 861 | 79 | 861 | 212.75 |
| HMGB2-PREX1 | 6.6E−04 | 6.5E−04 | 1.5E−03 | 0.63 | 0.63 | 0.62 | 153 | 210 | 325 | 195.5 | 226.5 | 599 | 153 | 599 | 218.25 |
| NCOA4-TATDN1 | 5.8E−04 | 2.1E−04 | 2.4E−05 | 0.61 | 0.62 | 0.63 | 136 | 70 | 6 | 449 | 422.5 | 302 | 6 | 449 | 219 |
| MRPL42-HEATR3 | 6.3E−03 | 1.6E−03 | 3.6E−03 | 0.66 | 0.67 | 0.67 | 880 | 399 | 614 | 8.5 | 4 | 39.5 | 4 | 880 | 219.25 |
| AEBP1-URM1 | 8.5E−04 | 2.2E−03 | 9.2E−04 | 0.64 | 0.63 | 0.63 | 206 | 499 | 213 | 116.5 | 226.5 | 434 | 116.5 | 499 | 219.75 |
| GINS1-LYRM5 | 5.1E−05 | 4.3E−05 | 2.3E−04 | 0.62 | 0.62 | 0.62 | 15 | 12 | 64 | 386.5 | 377 | 784 | 12 | 784 | 220.5 |
| CALM3-HEATR3 | 3.9E−03 | 9.7E−04 | 3.1E−03 | 0.63 | 0.64 | 0.65 | 653 | 282 | 553 | 164 | 77.5 | 145 | 77.5 | 653 | 223 |
| TMEM49-SNUPN | 3.6E−04 | 6.1E−04 | 7.2E−04 | 0.62 | 0.62 | 0.62 | 84 | 201 | 174 | 245.5 | 467 | 683.5 | 84 | 683.5 | 223.25 |
| SNX27-MPP6 | 9.8E−04 | 2.0E−03 | 2.6E−04 | 0.63 | 0.62 | 0.64 | 225 | 468 | 71 | 172 | 355.5 | 222.5 | 71 | 468 | 223.75 |
| PREX1-C8orf33 | 1.7E−03 | 1.7E−03 | 9.3E−04 | 0.63 | 0.63 | 0.64 | 369 | 423 | 214 | 189.5 | 238.5 | 198.5 | 189.5 | 423 | 226.25 |
| TMEM49-MPP6 | 1.7E−04 | 5.6E−04 | 3.5E−05 | 0.62 | 0.61 | 0.62 | 41 | 187 | 11 | 271 | 571 | 599 | 11 | 599 | 229 |
| HEMK1-GINS1 | 5.0E−04 | 2.7E−04 | 6.0E−04 | 0.62 | 0.62 | 0.63 | 120 | 95 | 151 | 308.5 | 355.5 | 449.5 | 95 | 449.5 | 229.75 |
| SMARCB1-SEC14L1 | 1.3E−02 | 5.9E−03 | 1.8E−03 | 0.64 | 0.65 | 0.66 | 1481 | 955 | 364 | 98 | 52.5 | 94 | 52.5 | 1481 | 231 |
| TMEM8B-SEC14L1 | 1.0E−03 | 4.6E−04 | 1.2E−04 | 0.61 | 0.62 | 0.64 | 236 | 149 | 34 | 807.5 | 366 | 231.5 | 34 | 807.5 | 233.75 |
| SELM-C5orf62 | 1.4E−02 | 9.2E−03 | 1.5E−03 | 0.64 | 0.64 | 0.66 | 1533 | 1257 | 322 | 103.5 | 147.5 | 60 | 60 | 1533 | 234.75 |
| RPL37-C5orf62 | 1.7E−02 | 1.1E−02 | 1.8E−03 | 0.64 | 0.64 | 0.66 | 1678 | 1436 | 379 | 93.5 | 81.5 | 80 | 80 | 1678 | 236.25 |
| SEC14L1-YY1AP1 | 6.0E−03 | 3.1E−03 | 1.1E−03 | 0.62 | 0.64 | 0.64 | 850 | 619 | 258 | 215.5 | 155.5 | 206.5 | 155.5 | 850 | 236.75 |
| ZFAND5-C20orf111 | 1.3E−03 | 7.3E−04 | 1.8E−04 | 0.62 | 0.63 | 0.64 | 291 | 234 | 54 | 245.5 | 181 | 247.5 | 54 | 291 | 239.75 |
| ABHD12-CALM3 | 9.6E−04 | 1.6E−03 | 1.7E−04 | 0.62 | 0.62 | 0.66 | 220 | 397 | 48 | 261.5 | 440 | 88 | 48 | 440 | 240.75 |
| TMEM8B-C16orf53 | 3.8E−03 | 1.7E−03 | 2.6E−03 | 0.65 | 0.66 | 0.66 | 638 | 416 | 488 | 28 | 13 | 66 | 13 | 638 | 241 |
| TMEM49-MT1E | 1.9E−04 | 1.0E−04 | 1.9E−03 | 0.62 | 0.63 | 0.62 | 44 | 29 | 383 | 294.5 | 200.5 | 784 | 29 | 784 | 247.5 |
| MAST2-MT1E | 3.7E−04 | 1.6E−04 | 2.1E−03 | 0.62 | 0.63 | 0.62 | 87 | 44 | 406 | 294.5 | 200.5 | 570.5 | 44 | 570.5 | 247.5 |
| PREX1-AAK1 | 8.2E−03 | 7.8E−03 | 6.3E−04 | 0.62 | 0.63 | 0.66 | 1045 | 1143 | 161 | 294.5 | 200.5 | 53.5 | 53.5 | 1143 | 247.5 |
| MT1E-SARM1 | 7.5E−04 | 3.2E−04 | 6.2E−03 | 0.62 | 0.63 | 0.62 | 178 | 109 | 919 | 294.5 | 200.5 | 784 | 109 | 919 | 247.5 |
| ABHD12-RPS20 | 1.7E−03 | 1.4E−03 | 5.0E−04 | 0.62 | 0.63 | 0.65 | 364 | 365 | 129 | 308.5 | 189 | 162.5 | 129 | 365 | 248.75 |
| AEBP1-NSUN5 | 1.4E−04 | 4.5E−04 | 3.1E−04 | 0.62 | 0.61 | 0.61 | 37 | 148 | 86 | 350 | 749.5 | 897.5 | 37 | 897.5 | 249 |
| TMEM49-CALM3 | 2.2E−05 | 1.2E−04 | 1.7E−04 | 0.61 | 0.61 | 0.62 | 6 | 32 | 50 | 449 | 1000.5 | 599 | 6 | 1000.5 | 249.5 |
| LAPTM5-LYRM5 | 3.4E−03 | 5.2E−03 | 2.3E−03 | 0.66 | 0.66 | 0.66 | 586 | 886 | 447 | 17.5 | 18 | 60 | 17.5 | 886 | 253.5 |
| MT1E-SHMT1 | 9.1E−04 | 4.1E−04 | 8.4E−03 | 0.62 | 0.63 | 0.62 | 214 | 137 | 1123 | 294.5 | 200.5 | 784 | 137 | 1123 | 254.25 |
| VAMP2-AP2A1 | 3.0E−04 | 2.2E−04 | 1.2E−04 | 0.61 | 0.61 | 0.63 | 67 | 79 | 33 | 762.5 | 775 | 434 | 33 | 775 | 256.5 |
| NCOA4-SNUPN | 4.8E−03 | 2.8E−03 | 1.2E−03 | 0.63 | 0.63 | 0.64 | 748 | 587 | 266 | 181.5 | 253 | 180 | 180 | 748 | 259.5 |
| LAPTM5-RPL19 | 4.0E−03 | 3.4E−03 | 1.7E−03 | 0.63 | 0.64 | 0.65 | 668 | 669 | 352 | 172 | 104.5 | 162.5 | 104.5 | 669 | 262 |
| VAMP2-CCDC6 | 9.8E−04 | 3.9E−04 | 1.0E−04 | 0.61 | 0.62 | 0.63 | 226 | 134 | 29 | 595 | 316.5 | 302 | 29 | 595 | 264 |
| PREX1-LYRM5 | 1.2E−03 | 1.2E−03 | 6.3E−04 | 0.62 | 0.63 | 0.63 | 280 | 331 | 160 | 245.5 | 253 | 499.5 | 160 | 499.5 | 266.5 |
| PREX1-SMARCB1 | 4.1E−03 | 2.2E−03 | 2.2E−03 | 0.64 | 0.64 | 0.66 | 672 | 498 | 426 | 108 | 98 | 44 | 44 | 672 | 267 |
| RPL37-MAF | 1.4E−03 | 1.1E−03 | 5.1E−03 | 0.64 | 0.64 | 0.64 | 322 | 310 | 788 | 89 | 85 | 229 | 85 | 788 | 269.5 |
| GINS1-RPS20 | 2.0E−04 | 1.2E−04 | 5.5E−04 | 0.62 | 0.62 | 0.61 | 46 | 34 | 139 | 402 | 495.5 | 1164.5 | 34 | 1164.5 | 270.5 |
| XPO1-SEC14L1 | 2.0E−02 | 1.6E−02 | 2.5E−03 | 0.64 | 0.65 | 0.67 | 1837 | 1679 | 480 | 64 | 52.5 | 32 | 32 | 1837 | 272 |
| MRPL42-CCDC6 | 1.6E−02 | 8.1E−03 | 2.0E−03 | 0.63 | 0.64 | 0.66 | 1645 | 1171 | 402 | 147.5 | 124 | 66 | 66 | 1645 | 274.75 |
| RCHY1-MT1E | 1.2E−03 | 5.2E−04 | 5.1E−03 | 0.62 | 0.63 | 0.62 | 264 | 168 | 786 | 285.5 | 209.5 | 517 | 168 | 786 | 274.75 |
| PRPF3-MT1E | 1.2E−03 | 5.2E−04 | 7.3E−03 | 0.62 | 0.63 | 0.61 | 266 | 167 | 1030 | 285.5 | 209.5 | 958 | 167 | 1030 | 275.75 |
| GINS1-CALM3 | 6.3E−05 | 5.1E−05 | 2.4E−04 | 0.61 | 0.62 | 0.62 | 17 | 18 | 65 | 527 | 495.5 | 623.5 | 17 | 623.5 | 280.25 |
| GCH1-C20orf111 | 1.4E−03 | 3.2E−03 | 3.1E−03 | 0.64 | 0.64 | 0.64 | 313 | 645 | 560 | 74.5 | 138 | 247.5 | 74.5 | 645 | 280.25 |
| AEBP1-PDCD6IP | 8.0E−04 | 2.5E−03 | 9.8E−04 | 0.63 | 0.62 | 0.63 | 190 | 541 | 226 | 139 | 337.5 | 349 | 139 | 541 | 281.75 |
| NCOA4-C20orf111 | 5.7E−04 | 4.7E−04 | 3.5E−04 | 0.62 | 0.62 | 0.63 | 133 | 151 | 96 | 417.5 | 467 | 499.5 | 96 | 499.5 | 284.25 |
| TMEM8B-VTI1B | 1.1E−03 | 7.7E−04 | 5.4E−04 | 0.61 | 0.61 | 0.63 | 240 | 242 | 136 | 606 | 749.5 | 330.5 | 136 | 749.5 | 286.25 |
| MT1E-RNPEP | 4.7E−04 | 2.8E−04 | 2.8E−03 | 0.62 | 0.63 | 0.61 | 115 | 99 | 514 | 294.5 | 285 | 1068 | 99 | 1068 | 289.75 |
| MT1E-C5orf32 | 8.6E−05 | 4.9E−05 | 9.0E−04 | 0.62 | 0.62 | 0.61 | 21 | 16 | 207 | 373.5 | 393 | 1276.5 | 16 | 1276.5 | 290.25 |
| FOXN2-LYRM5 | 1.6E−03 | 1.7E−03 | 7.2E−04 | 0.62 | 0.63 | 0.64 | 345 | 424 | 173 | 435 | 238.5 | 198.5 | 173 | 435 | 291.75 |
| LYRM5-C5orf62 | 8.2E−03 | 7.5E−03 | 1.0E−03 | 0.62 | 0.62 | 0.65 | 1061 | 1110 | 233 | 229 | 355.5 | 162.5 | 162.5 | 1110 | 294.25 |
| SEC14L1-HNRNPD | 2.2E−03 | 1.3E−03 | 1.1E−03 | 0.63 | 0.63 | 0.63 | 455 | 348 | 241 | 181.5 | 181 | 499.5 | 181 | 499.5 | 294.5 |
| MRPL42-SEC14L1 | 1.4E−02 | 8.4E−03 | 2.5E−03 | 0.64 | 0.65 | 0.66 | 1525 | 1194 | 479 | 112.5 | 64.5 | 50 | 50 | 1525 | 295.75 |
| TMEM49-TMEM8B | 1.2E−03 | 1.1E−03 | 9.0E−04 | 0.63 | 0.62 | 0.62 | 277 | 317 | 208 | 151 | 345 | 520.5 | 151 | 520.5 | 297 |
| VAMP2-SERBP1 | 6.8E−04 | 2.0E−04 | 1.4E−04 | 0.61 | 0.62 | 0.61 | 157 | 67 | 38 | 579 | 440 | 1027.5 | 38 | 1027.5 | 298.5 |
| TMEM8B-SPG7 | 2.3E−03 | 6.6E−04 | 7.5E−04 | 0.61 | 0.63 | 0.63 | 468 | 218 | 182 | 466.5 | 276 | 324 | 182 | 468 | 300 |
| LYRM5-TPT1 | 1.9E−03 | 2.6E−03 | 1.7E−03 | 0.64 | 0.64 | 0.64 | 405 | 550 | 353 | 74.5 | 98 | 247.5 | 74.5 | 550 | 300.25 |
| NSUN5-MPP6 | 8.5E−04 | 9.2E−04 | 3.5E−04 | 0.62 | 0.62 | 0.63 | 207 | 269 | 97 | 417.5 | 337.5 | 349 | 97 | 417.5 | 303.25 |
| MRPL42-FOXN2 | 5.5E−03 | 4.0E−03 | 2.4E−03 | 0.63 | 0.65 | 0.67 | 815 | 742 | 460 | 147.5 | 63 | 23.5 | 23.5 | 815 | 303.75 |
| SELM-AP2A1 | 5.1E−03 | 3.2E−03 | 1.7E−03 | 0.62 | 0.63 | 0.65 | 772 | 635 | 346 | 261.5 | 177.5 | 133 | 133 | 772 | 303.75 |
| AEBP1-ABHD12 | 7.0E−04 | 2.1E−03 | 2.7E−04 | 0.62 | 0.62 | 0.64 | 164 | 472 | 76 | 355 | 544 | 271.5 | 76 | 544 | 313.25 |
| AEBP1-SERPINB9 | 6.2E−04 | 1.4E−03 | 6.0E−04 | 0.62 | 0.62 | 0.62 | 144 | 370 | 155 | 261.5 | 440 | 547.5 | 144 | 547.5 | 315.75 |
| IL1RN-MT1G | 1.6E−03 | 9.8E−04 | 1.3E−02 | 0.63 | 0.64 | 0.62 | 354 | 283 | 1450 | 164 | 155.5 | 570.5 | 155.5 | 1450 | 318.5 |
| ABHD12-RPL19 | 1.5E−03 | 1.0E−03 | 2.6E−04 | 0.62 | 0.62 | 0.64 | 338 | 300 | 72 | 417.5 | 337.5 | 180 | 72 | 417.5 | 318.75 |
| PREX1-MT1E | 2.6E−04 | 1.2E−04 | 1.7E−03 | 0.62 | 0.63 | 0.61 | 57 | 33 | 356 | 386.5 | 285 | 1068 | 33 | 1068 | 320.5 |
| GINS1-KLF12 | 2.6E−04 | 1.8E−04 | 5.7E−04 | 0.61 | 0.62 | 0.62 | 59 | 54 | 145 | 501.5 | 521.5 | 784 | 54 | 784 | 323.25 |
| CBX5-PREX1 | 1.9E−03 | 1.6E−03 | 5.4E−04 | 0.62 | 0.63 | 0.64 | 406 | 394 | 137 | 417.5 | 253 | 247.5 | 137 | 417.5 | 323.5 |
| SNX27-TMEM8B | 1.5E−03 | 1.1E−03 | 8.4E−04 | 0.62 | 0.62 | 0.62 | 326 | 321 | 198 | 276 | 449 | 578 | 198 | 578 | 323.5 |
| TMEM8B-AP2A1 | 2.8E−03 | 1.9E−03 | 1.1E−03 | 0.62 | 0.63 | 0.65 | 521 | 451 | 255 | 357 | 293.5 | 153 | 153 | 521 | 325.25 |
| VAMP2-STAM | 9.6E−04 | 5.2E−04 | 3.8E−04 | 0.62 | 0.61 | 0.60 | 221 | 166 | 103 | 435 | 803 | 1352 | 103 | 1352 | 328 |
| SMNDC1-PREX1 | 2.7E−03 | 3.3E−03 | 5.5E−03 | 0.64 | 0.65 | 0.65 | 512 | 657 | 846 | 48.5 | 52.5 | 145 | 48.5 | 846 | 328.5 |
| AEBP1-DGKA | 1.5E−03 | 4.7E−03 | 4.4E−03 | 0.64 | 0.63 | 0.63 | 341 | 823 | 699 | 68 | 268.5 | 316.5 | 68 | 823 | 328.75 |
| HMGB2-C5orf32 | 1.1E−03 | 2.0E−03 | 4.9E−03 | 0.64 | 0.64 | 0.63 | 251 | 457 | 760 | 48.5 | 111.5 | 408 | 48.5 | 760 | 329.5 |
| ERMP1-GINS1 | 2.1E−03 | 1.5E−03 | 2.3E−03 | 0.63 | 0.64 | 0.64 | 429 | 374 | 434 | 164 | 111.5 | 288 | 111.5 | 434 | 331 |
| AEBP1-C16orf53 | 4.1E−04 | 1.1E−03 | 9.7E−04 | 0.62 | 0.61 | 0.62 | 101 | 320 | 223 | 344 | 775 | 599 | 101 | 775 | 332 |
| XRCC1-SNX27 | 1.6E−03 | 4.3E−03 | 2.4E−03 | 0.63 | 0.63 | 0.63 | 351 | 768 | 461 | 134.5 | 268.5 | 316.5 | 134.5 | 768 | 333.75 |
| MT1E-DPP7 | 6.5E−04 | 3.7E−04 | 5.5E−03 | 0.62 | 0.63 | 0.61 | 151 | 128 | 835 | 386.5 | 285 | 1068 | 128 | 1068 | 335.75 |
| FGFR1OP2-MT1E | 1.0E−03 | 4.1E−04 | 3.3E−03 | 0.62 | 0.63 | 0.61 | 232 | 139 | 584 | 386.5 | 285 | 1068 | 139 | 1068 | 335.75 |
| MT1E-IKBKB | 1.2E−03 | 5.7E−04 | 9.0E−03 | 0.62 | 0.63 | 0.62 | 284 | 190 | 1177 | 386.5 | 285 | 784 | 190 | 1177 | 335.75 |
| C5orf32-MT1H | 5.4E−04 | 5.2E−04 | 9.8E−03 | 0.62 | 0.62 | 0.60 | 126 | 169 | 1248 | 363.5 | 311.5 | 1828.5 | 126 | 1828.5 | 337.5 |
| AEBP1-FOXN2 | 1.1E−03 | 3.3E−03 | 8.6E−04 | 0.62 | 0.61 | 0.63 | 253 | 650 | 200 | 350 | 749.5 | 330.5 | 200 | 749.5 | 340.25 |
| MT1E-CH25H | 2.6E−04 | 8.0E−05 | 9.5E−04 | 0.61 | 0.62 | 0.60 | 56 | 21 | 217 | 705.5 | 467 | 1635 | 21 | 1635 | 342 |
| TMEM8B-SERPINB9 | 1.2E−03 | 5.5E−04 | 4.7E−04 | 0.60 | 0.60 | 0.63 | 265 | 185 | 124 | 1036 | 1179.5 | 421.5 | 124 | 1179.5 | 343.25 |
| NCOA4-SMARCB1 | 5.2E−03 | 2.2E−03 | 1.8E−03 | 0.62 | 0.63 | 0.63 | 780 | 492 | 372 | 308.5 | 189 | 316.5 | 189 | 780 | 344.25 |
| XRCC1-GINS1 | 2.3E−04 | 2.2E−04 | 8.2E−04 | 0.61 | 0.61 | 0.61 | 51 | 76 | 194 | 501.5 | 695.5 | 1068 | 51 | 1068 | 347.75 |
| MAF-RPL19 | 3.3E−04 | 2.3E−04 | 1.1E−03 | 0.61 | 0.62 | 0.60 | 75 | 80 | 256 | 579 | 440 | 1352 | 75 | 1352 | 348 |
| MAST2-MT1H | 1.7E−03 | 1.2E−03 | 1.7E−02 | 0.64 | 0.64 | 0.63 | 368 | 335 | 1704 | 85 | 70 | 426 | 70 | 1704 | 351.5 |
| TMEM8B-EIF4H | 3.7E−03 | 2.2E−03 | 1.7E−03 | 0.62 | 0.63 | 0.65 | 628 | 506 | 354 | 350 | 216.5 | 114 | 114 | 628 | 352 |
| RPL37-SEC14L1 | 1.6E−02 | 7.7E−03 | 2.4E−03 | 0.62 | 0.64 | 0.64 | 1642 | 1133 | 463 | 245.5 | 98 | 180 | 98 | 1642 | 354.25 |
| VAMP2-EIF4H | 2.6E−03 | 1.5E−03 | 1.7E−03 | 0.62 | 0.63 | 0.63 | 492 | 384 | 344 | 285.5 | 165.5 | 365.5 | 165.5 | 492 | 354.75 |
| VAMP2-AKIRIN2 | 1.9E−03 | 1.9E−03 | 2.6E−04 | 0.62 | 0.62 | 0.64 | 404 | 450 | 74 | 308.5 | 495.5 | 222.5 | 74 | 495.5 | 356.25 |
| VAMP2-GPBAR1 | 1.2E−03 | 1.4E−03 | 4.1E−04 | 0.62 | 0.62 | 0.63 | 279 | 363 | 107 | 402 | 355.5 | 449.5 | 107 | 449.5 | 359.25 |
| SMYD3-SEC14L1 | 1.2E−02 | 1.0E−02 | 1.8E−03 | 0.62 | 0.62 | 0.65 | 1365 | 1359 | 382 | 323.5 | 337.5 | 119 | 119 | 1365 | 359.75 |
| PLAC8-GINS1 | 1.1E−04 | 8.1E−05 | 2.5E−04 | 0.61 | 0.61 | 0.60 | 24 | 22 | 69 | 654.5 | 905 | 1810 | 22 | 1810 | 361.75 |
| TMEM8B-SIPA1L2 | 5.4E−03 | 3.8E−03 | 3.6E−03 | 0.63 | 0.64 | 0.65 | 809 | 709 | 610 | 119 | 122.5 | 111.5 | 111.5 | 809 | 366.25 |
| RABL2B-GINS1 | 4.2E−05 | 3.1E−05 | 1.7E−04 | 0.60 | 0.61 | 0.62 | 14 | 10 | 49 | 846.5 | 695.5 | 784 | 10 | 846.5 | 372.25 |
| FOXN2-RPL19 | 4.7E−03 | 2.7E−03 | 1.8E−03 | 0.62 | 0.63 | 0.66 | 732 | 575 | 368 | 386.5 | 200.5 | 94 | 94 | 732 | 377.25 |
| CRTAP-RPS20 | 2.4E−03 | 1.8E−03 | 9.8E−04 | 0.62 | 0.62 | 0.64 | 479 | 442 | 227 | 435 | 324 | 198.5 | 198.5 | 479 | 379.5 |
| MAF-MT1E | 5.6E−04 | 3.0E−04 | 4.6E−03 | 0.62 | 0.62 | 0.60 | 131 | 104 | 721 | 386.5 | 377 | 1391.5 | 104 | 1391.5 | 381.75 |
| TMEM8B-ZFAND5 | 4.1E−03 | 3.1E−03 | 1.2E−03 | 0.61 | 0.63 | 0.64 | 679 | 622 | 268 | 462 | 302.5 | 193 | 193 | 679 | 382.25 |
| ADRB2-MT1H | 6.7E−04 | 5.7E−04 | 5.0E−03 | 0.62 | 0.62 | 0.61 | 156 | 191 | 770 | 373.5 | 393 | 1276.5 | 156 | 1276.5 | 383.25 |
| VAMP2-CTCF | 4.7E−03 | 1.6E−03 | 1.9E−03 | 0.62 | 0.64 | 0.62 | 731 | 393 | 393 | 215.5 | 111.5 | 570.5 | 111.5 | 731 | 393 |
| ANXA11-GINS1 | 7.1E−04 | 5.0E−04 | 2.1E−03 | 0.62 | 0.61 | 0.61 | 170 | 164 | 415 | 373.5 | 714.5 | 1276.5 | 164 | 1276.5 | 394.25 |
| RPUSD1-PRKAR1B | 6.5E−04 | 1.4E−03 | 5.7E−04 | 0.59 | 0.59 | 0.63 | 149 | 368 | 142 | 1563 | 1972 | 421.5 | 142 | 1972 | 394.75 |
| AEBP1-CCDC6 | 2.2E−03 | 4.5E−03 | 7.2E−04 | 0.62 | 0.62 | 0.64 | 442 | 798 | 172 | 350 | 554 | 235 | 172 | 798 | 396 |
| PREX1-MRPL42 | 8.0E−03 | 6.1E−03 | 4.2E−03 | 0.64 | 0.64 | 0.66 | 1030 | 986 | 681 | 112.5 | 90.5 | 88 | 88 | 1030 | 396.75 |
| LYRM5-GPBAR1 | 7.6E−03 | 1.1E−02 | 2.8E−03 | 0.62 | 0.63 | 0.66 | 998 | 1434 | 512 | 215.5 | 285 | 94 | 94 | 1434 | 398.5 |
| NCOA4-RPS20 | 6.2E−04 | 3.4E−04 | 2.0E−04 | 0.59 | 0.60 | 0.62 | 143 | 118 | 58 | 1802 | 1236 | 655.5 | 58 | 1802 | 399.25 |
| FOXN2-MPP6 | 1.7E−03 | 2.5E−03 | 9.1E−05 | 0.62 | 0.61 | 0.66 | 366 | 539 | 27 | 435 | 592 | 88 | 27 | 592 | 400.5 |
| SEC14L1-AKIRIN2 | 1.4E−02 | 6.0E−03 | 4.6E−03 | 0.64 | 0.66 | 0.66 | 1531 | 975 | 718 | 48.5 | 20.5 | 94 | 20.5 | 1531 | 406 |
| SELM-SEC14L1 | 6.5E−03 | 2.9E−03 | 6.0E−04 | 0.62 | 0.63 | 0.63 | 901 | 601 | 152 | 435 | 238.5 | 389.5 | 152 | 901 | 412.25 |
| LYRM5-DPP7 | 2.4E−03 | 4.0E−03 | 1.0E−02 | 0.64 | 0.64 | 0.63 | 481 | 729 | 1282 | 74.5 | 98 | 349 | 74.5 | 1282 | 415 |
| HMGB2-C5orf62 | 1.2E−02 | 1.2E−02 | 4.0E−03 | 0.64 | 0.63 | 0.64 | 1410 | 1448 | 651 | 93.5 | 165.5 | 188 | 93.5 | 1448 | 419.5 |
| AEBP1-TPT1 | 2.2E−03 | 6.9E−03 | 2.7E−03 | 0.64 | 0.63 | 0.63 | 450 | 1055 | 509 | 112.5 | 238.5 | 389.5 | 112.5 | 1055 | 419.75 |
| CRTAP-SMARCB1 | 1.3E−02 | 7.8E−03 | 5.0E−03 | 0.64 | 0.65 | 0.66 | 1467 | 1138 | 774 | 55.5 | 67.5 | 72 | 55.5 | 1467 | 423 |
| SNX27-DGKA | 8.7E−03 | 1.8E−02 | 4.6E−03 | 0.64 | 0.64 | 0.65 | 1112 | 1798 | 712 | 108 | 138 | 119 | 108 | 1798 | 425 |
| TMEM49-SMARCB1 | 4.5E−03 | 5.4E−03 | 9.3E−03 | 0.63 | 0.64 | 0.65 | 714 | 902 | 1210 | 142.5 | 129.5 | 133 | 129.5 | 1210 | 428.25 |
| AEBP1-CH25H | 1.8E−03 | 1.6E−03 | 7.5E−04 | 0.61 | 0.61 | 0.63 | 391 | 395 | 179 | 807.5 | 896.5 | 464.5 | 179 | 896.5 | 429.75 |
| NCOA4-MPP5 | 2.0E−03 | 1.9E−03 | 4.3E−04 | 0.61 | 0.61 | 0.64 | 419 | 444 | 112 | 736 | 592 | 279.5 | 112 | 736 | 431.5 |
| NDUFC1-GINS1 | 4.4E−04 | 3.1E−04 | 1.0E−03 | 0.61 | 0.61 | 0.62 | 106 | 108 | 240 | 705.5 | 624.5 | 683.5 | 106 | 705.5 | 432.25 |
| NSUN5-KLF12 | 6.2E−03 | 3.9E−03 | 8.6E−03 | 0.64 | 0.65 | 0.65 | 869 | 720 | 1143 | 64 | 31.5 | 145 | 31.5 | 1143 | 432.5 |
| TATDN1-GINS1 | 6.8E−05 | 3.3E−05 | 8.7E−05 | 0.60 | 0.61 | 0.61 | 18 | 11 | 24 | 1128.5 | 843 | 934.5 | 11 | 1128.5 | 433.5 |
| VAMP2-GCA | 1.9E−02 | 1.5E−02 | 3.9E−03 | 0.63 | 0.63 | 0.64 | 1782 | 1632 | 638 | 154.5 | 171.5 | 229 | 154.5 | 1782 | 433.5 |
| TATDN1-C5orf62 | 1.5E−02 | 9.8E−03 | 1.2E−03 | 0.61 | 0.62 | 0.65 | 1590 | 1309 | 264 | 449 | 422.5 | 100 | 100 | 1590 | 435.75 |
| HMGB2-URM1 | 2.5E−03 | 1.6E−03 | 1.4E−02 | 0.63 | 0.63 | 0.62 | 483 | 389 | 1554 | 172 | 189 | 623.5 | 172 | 1554 | 436 |
| SNX27-MT1E | 3.7E−04 | 1.9E−04 | 2.7E−03 | 0.61 | 0.62 | 0.60 | 88 | 61 | 500 | 501.5 | 377 | 1391.5 | 61 | 1391.5 | 438.5 |
| TMEM8B-NSUN5 | 5.1E−03 | 2.6E−03 | 4.3E−03 | 0.63 | 0.64 | 0.63 | 775 | 554 | 686 | 150 | 75 | 324 | 75 | 775 | 439 |
| ABHD12-SEPT6 | 1.3E−02 | 2.1E−02 | 3.6E−03 | 0.62 | 0.63 | 0.65 | 1477 | 1980 | 607 | 271 | 226.5 | 100 | 100 | 1980 | 439 |
| NSUN5-MT1E | 7.6E−04 | 3.5E−04 | 5.6E−03 | 0.61 | 0.62 | 0.60 | 181 | 124 | 860 | 501.5 | 377 | 1391.5 | 124 | 1391.5 | 439.25 |
| MT1E-C7orf68 | 1.1E−03 | 3.9E−04 | 5.4E−03 | 0.61 | 0.62 | 0.60 | 238 | 136 | 832 | 501.5 | 377 | 1391.5 | 136 | 1391.5 | 439.25 |
| MT1E-TIMM23 | 9.7E−04 | 4.4E−04 | 5.2E−03 | 0.61 | 0.62 | 0.61 | 223 | 147 | 797 | 501.5 | 377 | 1068 | 147 | 1068 | 439.25 |
| MT1E-RANBP1 | 8.7E−04 | 4.7E−04 | 7.3E−03 | 0.61 | 0.62 | 0.60 | 209 | 153 | 1034 | 501.5 | 377 | 1391.5 | 153 | 1391.5 | 439.25 |
| SMNDC1-MT1E | 1.2E−03 | 5.3E−04 | 6.0E−03 | 0.61 | 0.62 | 0.61 | 270 | 175 | 893 | 501.5 | 377 | 1068 | 175 | 1068 | 439.25 |
| MT1E-CD2BP2 | 1.1E−03 | 5.3E−04 | 7.7E−03 | 0.61 | 0.62 | 0.60 | 261 | 176 | 1067 | 501.5 | 377 | 1391.5 | 176 | 1391.5 | 439.25 |
| MT1E-DGKH | 7.7E−04 | 3.2E−04 | 4.2E−03 | 0.61 | 0.62 | 0.61 | 182 | 110 | 676 | 527 | 355.5 | 861 | 110 | 861 | 441.25 |
| SEC14L1-SNRPC | 1.2E−02 | 6.4E−03 | 4.7E−03 | 0.63 | 0.64 | 0.65 | 1407 | 1019 | 729 | 134.5 | 104.5 | 162.5 | 104.5 | 1407 | 445.75 |
| PSMD7-ABHD12 | 4.5E−03 | 6.1E−03 | 5.0E−03 | 0.63 | 0.64 | 0.65 | 713 | 984 | 766 | 181.5 | 138 | 119 | 119 | 984 | 447.25 |
| SMARCB1-GINS1 | 4.1E−04 | 2.0E−04 | 1.2E−03 | 0.60 | 0.61 | 0.62 | 100 | 68 | 275 | 917.5 | 624.5 | 683.5 | 68 | 917.5 | 449.75 |
| SNX27-SNURF | 4.1E−03 | 6.0E−03 | 4.9E−03 | 0.63 | 0.63 | 0.65 | 677 | 973 | 753 | 195.5 | 226.5 | 100 | 100 | 973 | 451.75 |
| SEC14L1-CRLF3 | 1.7E−02 | 1.7E−02 | 5.0E−03 | 0.63 | 0.64 | 0.66 | 1709 | 1770 | 771 | 139 | 98 | 44 | 44 | 1770 | 455 |
| SEC14L1-YPEL5 | 4.6E−03 | 2.9E−03 | 3.5E−03 | 0.64 | 0.65 | 0.63 | 724 | 595 | 604 | 52.5 | 45.5 | 316.5 | 45.5 | 724 | 455.75 |
| NCOA4-MRPL42 | 1.3E−03 | 7.4E−04 | 3.2E−04 | 0.61 | 0.61 | 0.62 | 311 | 235 | 88 | 617 | 708.5 | 808 | 88 | 808 | 464 |
| C5orf62-TMEM167B | 1.3E−02 | 1.6E−02 | 3.8E−03 | 0.63 | 0.63 | 0.64 | 1423 | 1672 | 631 | 158 | 297.5 | 262 | 158 | 1672 | 464.25 |
| NCOA4-CALM3 | 7.9E−04 | 8.2E−04 | 3.0E−04 | 0.60 | 0.60 | 0.62 | 188 | 247 | 85 | 917.5 | 1100 | 683.5 | 85 | 1100 | 465.25 |
| VAMP2-HEATR3 | 2.1E−03 | 4.8E−04 | 1.4E−03 | 0.60 | 0.62 | 0.61 | 436 | 160 | 300 | 875.5 | 495.5 | 1164.5 | 160 | 1164.5 | 465.75 |
| CRTAP-TATDN1 | 2.7E−03 | 1.5E−03 | 2.4E−04 | 0.61 | 0.61 | 0.63 | 509 | 382 | 66 | 762.5 | 775 | 434 | 66 | 775 | 471.5 |
| VAMP2-UBXN4 | 9.3E−03 | 5.7E−03 | 2.2E−03 | 0.61 | 0.62 | 0.65 | 1162 | 941 | 422 | 527 | 355.5 | 162.5 | 162.5 | 1162 | 474.5 |
| C20orf111-C5orf32 | 1.5E−03 | 2.6E−03 | 4.2E−03 | 0.63 | 0.62 | 0.61 | 333 | 548 | 685 | 154.5 | 401.5 | 874 | 154.5 | 874 | 474.75 |
| VTI1B-MT1E | 4.3E−04 | 2.4E−04 | 2.4E−03 | 0.61 | 0.62 | 0.60 | 103 | 85 | 455 | 527 | 495.5 | 1513 | 85 | 1513 | 475.25 |
| AEBP1-MAST2 | 7.4E−04 | 1.8E−03 | 7.2E−04 | 0.61 | 0.60 | 0.61 | 175 | 432 | 175 | 527 | 1394.5 | 1164.5 | 175 | 1394.5 | 479.5 |
| NCOA4-RPL19 | 8.0E−04 | 3.3E−04 | 1.6E−04 | 0.60 | 0.61 | 0.61 | 193 | 115 | 43 | 1230.5 | 775 | 834.5 | 43 | 1230.5 | 484 |
| MPP6-NR2F6 | 1.4E−02 | 1.8E−02 | 4.4E−03 | 0.62 | 0.63 | 0.65 | 1548 | 1797 | 702 | 229 | 268.5 | 162.5 | 162.5 | 1797 | 485.25 |
| NCOA4-HMGB2 | 7.6E−04 | 5.9E−04 | 1.1E−03 | 0.61 | 0.61 | 0.60 | 179 | 197 | 243 | 736 | 1048.5 | 1766.5 | 179 | 1766.5 | 489.5 |
| SERPINB9-PRKAR1B | 9.3E−03 | 1.2E−02 | 4.1E−03 | 0.62 | 0.62 | 0.65 | 1168 | 1491 | 663 | 271 | 316.5 | 100 | 100 | 1491 | 489.75 |
| KIAA1949-TATDN1 | 3.8E−03 | 3.2E−03 | 6.4E−04 | 0.61 | 0.62 | 0.66 | 635 | 642 | 162 | 457 | 554 | 66 | 66 | 642 | 505.5 |
| SELM-EIF4H | 1.3E−02 | 7.0E−03 | 5.1E−03 | 0.62 | 0.63 | 0.64 | 1443 | 1064 | 785 | 229 | 189 | 222.5 | 189 | 1443 | 507 |
| NCOA4-PRKAR1B | 1.6E−03 | 2.5E−03 | 2.5E−04 | 0.60 | 0.59 | 0.63 | 356 | 540 | 68 | 1003 | 1756.5 | 475.5 | 68 | 1756.5 | 507.75 |
| SERPINB9-MT1E | 5.7E−04 | 2.7E−04 | 3.3E−03 | 0.61 | 0.62 | 0.60 | 132 | 94 | 579 | 501.5 | 521.5 | 1391.5 | 94 | 1391.5 | 511.5 |
| ABHD12-C20orf111 | 4.0E−04 | 5.8E−04 | 1.7E−04 | 0.60 | 0.61 | 0.61 | 97 | 195 | 46 | 980.5 | 1000.5 | 834.5 | 46 | 1000.5 | 514.75 |
| ABHD12-TATDN1 | 3.4E−03 | 2.1E−03 | 2.2E−04 | 0.61 | 0.61 | 0.64 | 583 | 479 | 62 | 579 | 592 | 198.5 | 62 | 592 | 529 |
| AEBP1-CRTAP | 7.0E−04 | 1.8E−03 | 3.6E−04 | 0.61 | 0.60 | 0.62 | 165 | 443 | 98 | 617 | 1179.5 | 808 | 98 | 1179.5 | 530 |
| SNX27-MRPL42 | 4.8E−03 | 5.6E−03 | 6.3E−03 | 0.62 | 0.63 | 0.63 | 746 | 923 | 922 | 229 | 189 | 316.5 | 189 | 923 | 531.25 |
| TTC21A-MT1E | 1.8E−03 | 5.6E−04 | 6.8E−03 | 0.61 | 0.62 | 0.61 | 392 | 188 | 971 | 678 | 355.5 | 1164.5 | 188 | 1164.5 | 535 |
| KLF12-DPP7 | 6.5E−03 | 9.9E−03 | 1.9E−02 | 0.64 | 0.64 | 0.65 | 902 | 1318 | 1845 | 89 | 122.5 | 171.5 | 89 | 1845 | 536.75 |
| KIAA1949-RPL19 | 2.2E−03 | 2.1E−03 | 8.1E−04 | 0.61 | 0.61 | 0.62 | 452 | 485 | 191 | 736 | 592 | 756 | 191 | 756 | 538.5 |
| NCOA4-LYRM5 | 3.5E−04 | 2.6E−04 | 8.6E−05 | 0.60 | 0.60 | 0.61 | 81 | 92 | 23 | 1023.5 | 1204.5 | 988 | 23 | 1204.5 | 540 |
| PLAC8-URM1 | 1.5E−03 | 9.3E−04 | 7.0E−04 | 0.60 | 0.61 | 0.62 | 334 | 272 | 170 | 1182 | 1048.5 | 756 | 170 | 1182 | 545 |
| NCDN-MT1E | 3.2E−04 | 1.3E−04 | 2.2E−03 | 0.60 | 0.61 | 0.60 | 71 | 36 | 430 | 875.5 | 665.5 | 1513 | 36 | 1513 | 547.75 |
| MT1E-GPBAR1 | 6.0E−04 | 3.5E−04 | 2.5E−03 | 0.60 | 0.61 | 0.60 | 141 | 122 | 475 | 875.5 | 665.5 | 1513 | 122 | 1513 | 570.25 |
| SEC14L1-BCL7B | 2.3E−02 | 1.0E−02 | 6.8E−03 | 0.63 | 0.64 | 0.66 | 1989 | 1345 | 972 | 172 | 72.5 | 60 | 60 | 1989 | 572 |
| RPUSD1-MT1E | 4.3E−04 | 2.2E−04 | 3.7E−03 | 0.61 | 0.62 | 0.60 | 105 | 75 | 627 | 654.5 | 521.5 | 1391.5 | 75 | 1391.5 | 574.25 |
| VAMP2-CCDC130 | 1.6E−02 | 7.7E−03 | 1.5E−02 | 0.66 | 0.67 | 0.67 | 1664 | 1134 | 1620 | 15 | 8 | 19 | 8 | 1664 | 576.5 |
| VAMP2-C16orf53 | 7.9E−03 | 4.6E−03 | 1.3E−02 | 0.62 | 0.64 | 0.63 | 1021 | 815 | 1477 | 245.5 | 138 | 349 | 138 | 1477 | 582 |
| PREX1-RPL26L1 | 6.6E−03 | 7.2E−03 | 7.8E−03 | 0.62 | 0.63 | 0.64 | 911 | 1085 | 1070 | 245.5 | 253 | 180 | 180 | 1085 | 582 |
| C8orf33-EIF4H | 1.0E−02 | 8.9E−03 | 4.9E−03 | 0.62 | 0.62 | 0.65 | 1245 | 1222 | 763 | 402 | 355.5 | 162.5 | 162.5 | 1245 | 582.5 |
| NCOA4-KLF12 | 3.6E−03 | 2.5E−03 | 6.0E−04 | 0.61 | 0.61 | 0.61 | 613 | 542 | 154 | 579 | 592 | 1027.5 | 154 | 1027.5 | 585.5 |
| LAPTM5-MT1E | 8.1E−04 | 4.3E−04 | 4.7E−03 | 0.61 | 0.62 | 0.60 | 194 | 144 | 730 | 678 | 495.5 | 1513 | 144 | 1513 | 586.75 |
| CC2D1B-MT1E | 1.3E−03 | 5.4E−04 | 8.3E−03 | 0.61 | 0.62 | 0.61 | 312 | 179 | 1120 | 678 | 495.5 | 1164.5 | 179 | 1164.5 | 586.75 |
| MT1E-STAM | 8.4E−04 | 4.1E−04 | 5.4E−03 | 0.61 | 0.62 | 0.60 | 202 | 141 | 828 | 654.5 | 521.5 | 1391.5 | 141 | 1391.5 | 588 |
| RARS2-MT1E | 1.0E−03 | 4.4E−04 | 5.2E−03 | 0.61 | 0.62 | 0.60 | 233 | 146 | 807 | 654.5 | 521.5 | 1391.5 | 146 | 1391.5 | 588 |
| MT1E-YPEL5 | 1.1E−03 | 4.9E−04 | 4.9E−03 | 0.61 | 0.62 | 0.60 | 245 | 163 | 764 | 654.5 | 521.5 | 1391.5 | 163 | 1391.5 | 588 |
| MT1E-C16orf53 | 1.1E−03 | 5.3E−04 | 7.3E−03 | 0.61 | 0.62 | 0.60 | 255 | 173 | 1024 | 654.5 | 521.5 | 1391.5 | 173 | 1391.5 | 588 |
| ANXA11-MT1E | 1.1E−03 | 5.4E−04 | 7.9E−03 | 0.61 | 0.62 | 0.60 | 254 | 177 | 1081 | 654.5 | 521.5 | 1810 | 177 | 1810 | 588 |
| MT1E-RAB35 | 4.7E−04 | 2.0E−04 | 2.8E−03 | 0.60 | 0.61 | 0.59 | 113 | 65 | 516 | 875.5 | 665.5 | 1963 | 65 | 1963 | 590.75 |
| VAMP2-SIPA1L2 | 1.0E−02 | 8.0E−03 | 1.1E−02 | 0.66 | 0.66 | 0.67 | 1246 | 1153 | 1299 | 14 | 16 | 34 | 14 | 1299 | 593.5 |
| TAF1-PREX1 | 8.2E−03 | 9.3E−03 | 8.4E−03 | 0.65 | 0.65 | 0.65 | 1053 | 1267 | 1132 | 22.5 | 31.5 | 145 | 22.5 | 1267 | 599 |
| AEBP1-C5orf62 | 2.2E−03 | 4.1E−03 | 4.3E−04 | 0.60 | 0.60 | 0.63 | 451 | 748 | 109 | 1182 | 1574.5 | 389.5 | 109 | 1574.5 | 599.5 |
| ABHD12-SMARCB1 | 3.5E−03 | 2.4E−03 | 7.0E−04 | 0.61 | 0.61 | 0.62 | 601 | 524 | 167 | 762.5 | 775 | 599 | 167 | 775 | 600 |
| LAPTM5-TATDN1 | 1.9E−02 | 1.6E−02 | 4.9E−03 | 0.61 | 0.62 | 0.66 | 1811 | 1673 | 751 | 462 | 398.5 | 84 | 84 | 1811 | 606.5 |
| ADRB2-SNUPN | 1.5E−02 | 1.8E−02 | 6.5E−03 | 0.63 | 0.63 | 0.65 | 1630 | 1807 | 949 | 172 | 268.5 | 107 | 107 | 1807 | 608.75 |
| MAF-KLF12 | 5.5E−03 | 6.1E−03 | 1.0E−02 | 0.63 | 0.64 | 0.63 | 817 | 985 | 1265 | 129.5 | 155.5 | 408 | 129.5 | 1265 | 612.5 |
| TMEM49-XRCC1 | 8.2E−04 | 3.3E−03 | 1.8E−03 | 0.61 | 0.60 | 0.61 | 200 | 647 | 361 | 579 | 1306 | 1027.5 | 200 | 1306 | 613 |
| AEBP1-DPP7 | 4.0E−04 | 1.8E−03 | 1.0E−03 | 0.61 | 0.59 | 0.60 | 96 | 441 | 238 | 796.5 | 1715.5 | 1310 | 96 | 1715.5 | 618.75 |
| PSMD7-SEC14L1 | 2.6E−03 | 1.7E−03 | 7.8E−04 | 0.60 | 0.61 | 0.61 | 506 | 410 | 188 | 1262 | 749.5 | 897.5 | 188 | 1262 | 627.75 |
| AEBP1-VTI1B | 2.7E−04 | 9.5E−04 | 4.4E−04 | 0.60 | 0.60 | 0.60 | 60 | 280 | 113 | 980.5 | 1514 | 1468.5 | 60 | 1514 | 630.25 |
| MPP6-C5orf32 | 4.7E−03 | 8.0E−03 | 1.9E−03 | 0.61 | 0.61 | 0.65 | 735 | 1157 | 386 | 527 | 881 | 162.5 | 162.5 | 1157 | 631 |
| SMYD3-GINS1 | 2.3E−04 | 2.0E−04 | 7.6E−04 | 0.60 | 0.60 | 0.61 | 50 | 66 | 185 | 1092 | 1140.5 | 1164.5 | 50 | 1164.5 | 638.5 |
| SELM-URM1 | 8.2E−04 | 1.9E−04 | 3.5E−04 | 0.59 | 0.60 | 0.60 | 196 | 59 | 93 | 1657.5 | 1100 | 1635 | 59 | 1657.5 | 648 |
| SIPA1L2-TATDN1 | 1.5E−02 | 1.0E−02 | 8.0E−03 | 0.62 | 0.63 | 0.64 | 1571 | 1344 | 1093 | 207 | 165.5 | 188 | 165.5 | 1571 | 650 |
| TATDN1-FOXN2 | 4.9E−03 | 2.7E−03 | 5.7E−04 | 0.60 | 0.60 | 0.64 | 762 | 561 | 144 | 1283.5 | 1204.5 | 193 | 144 | 1283.5 | 661.5 |
| MT1E-TMEM167B | 7.4E−04 | 3.0E−04 | 3.7E−03 | 0.60 | 0.61 | 0.60 | 176 | 105 | 628 | 846.5 | 695.5 | 1810 | 105 | 1810 | 661.75 |
| RPS20-RAB35 | 1.2E−02 | 6.7E−03 | 7.3E−03 | 0.62 | 0.64 | 0.64 | 1344 | 1045 | 1032 | 294.5 | 155.5 | 206.5 | 155.5 | 1344 | 663.25 |
| TMEM8B-GINS1 | 3.7E−04 | 2.0E−04 | 4.4E−04 | 0.60 | 0.60 | 0.60 | 90 | 62 | 114 | 1230.5 | 1263 | 1908.5 | 62 | 1908.5 | 672.25 |
| MT1E-UBXN4 | 9.1E−04 | 4.3E−04 | 4.4E−03 | 0.60 | 0.61 | 0.60 | 213 | 145 | 700 | 875.5 | 665.5 | 1513 | 145 | 1513 | 682.75 |
| SELM-C16orf53 | 1.8E−02 | 8.5E−03 | 2.1E−02 | 0.63 | 0.64 | 0.65 | 1779 | 1202 | 1943 | 164 | 111.5 | 145 | 111.5 | 1943 | 683 |
| TMEM49-RPS20 | 3.4E−04 | 5.1E−04 | 6.8E−04 | 0.59 | 0.59 | 0.61 | 77 | 165 | 166 | 1526 | 1756.5 | 1211 | 77 | 1756.5 | 688.5 |
| NPTN-MT1E | 5.2E−04 | 2.5E−04 | 4.4E−03 | 0.60 | 0.61 | 0.60 | 124 | 89 | 697 | 846.5 | 695.5 | 1810 | 89 | 1810 | 696.25 |
| SNX27-CALM3 | 6.7E−04 | 1.6E−03 | 2.0E−03 | 0.60 | 0.59 | 0.61 | 154 | 403 | 398 | 1003 | 1756.5 | 1211 | 154 | 1756.5 | 703 |
| AEBP1-KIAA1949 | 6.7E−04 | 2.4E−03 | 4.7E−04 | 0.60 | 0.59 | 0.61 | 155 | 521 | 121 | 1003 | 1756.5 | 897.5 | 121 | 1756.5 | 709.25 |
| GRK6-MT1E | 8.3E−04 | 3.8E−04 | 4.9E−03 | 0.60 | 0.61 | 0.60 | 201 | 131 | 758 | 875.5 | 665.5 | 1513 | 131 | 1513 | 711.75 |
| C20orf111-TIMM23 | 1.3E−02 | 9.1E−03 | 1.1E−02 | 0.63 | 0.64 | 0.64 | 1416 | 1243 | 1370 | 181.5 | 138 | 180 | 138 | 1416 | 712.25 |
| AEBP1-NR2F6 | 7.3E−04 | 1.9E−03 | 5.0E−04 | 0.60 | 0.59 | 0.61 | 171 | 447 | 130 | 980.5 | 1799 | 1132 | 130 | 1799 | 713.75 |
| TMEM8B-C5orf32 | 1.0E−03 | 8.8E−04 | 7.1E−04 | 0.59 | 0.60 | 0.61 | 229 | 258 | 171 | 1579 | 1275.5 | 1178 | 171 | 1579 | 718 |
| C20orf111-GINS1 | 2.4E−04 | 1.7E−04 | 7.5E−04 | 0.59 | 0.60 | 0.60 | 53 | 51 | 181 | 1495 | 1263 | 1908.5 | 51 | 1908.5 | 722 |
| SIPA1L2-PRKAR1B | 1.6E−02 | 1.9E−02 | 1.0E−02 | 0.64 | 0.63 | 0.66 | 1661 | 1891 | 1287 | 93.5 | 165.5 | 80 | 80 | 1891 | 726.25 |
| NPTN-PLAC8 | 8.6E−03 | 8.8E−03 | 1.3E−02 | 0.62 | 0.63 | 0.63 | 1104 | 1214 | 1490 | 245.5 | 181 | 349 | 181 | 1490 | 726.5 |
| TMEM8B-UBXN4 | 1.3E−02 | 7.4E−03 | 3.4E−03 | 0.61 | 0.61 | 0.64 | 1424 | 1104 | 585 | 810 | 645 | 191 | 191 | 1424 | 727.5 |
| MT1E-CDC37 | 8.2E−04 | 4.8E−04 | 5.3E−03 | 0.60 | 0.61 | 0.59 | 199 | 157 | 817 | 875.5 | 665.5 | 1963 | 157 | 1963 | 741.25 |
| TM9SF1-MT1E | 7.1E−04 | 3.4E−04 | 4.0E−03 | 0.60 | 0.61 | 0.60 | 167 | 121 | 647 | 1128.5 | 843 | 1635 | 121 | 1635 | 745 |
| ABHD12-PRKAR1B | 4.5E−03 | 8.3E−03 | 6.2E−04 | 0.61 | 0.60 | 0.63 | 711 | 1188 | 157 | 796.5 | 1450.5 | 373.5 | 157 | 1450.5 | 753.75 |
| FOXN2-SNURF | 1.8E−02 | 1.9E−02 | 9.9E−03 | 0.62 | 0.63 | 0.64 | 1744 | 1855 | 1250 | 261.5 | 238.5 | 198.5 | 198.5 | 1855 | 755.75 |
| RPL19-RAB35 | 2.0E−02 | 8.8E−03 | 9.0E−03 | 0.62 | 0.63 | 0.63 | 1849 | 1220 | 1172 | 285.5 | 165.5 | 365.5 | 165.5 | 1849 | 768.75 |
| MT1E-POLR3K | 1.1E−03 | 4.7E−04 | 7.8E−03 | 0.60 | 0.61 | 0.59 | 250 | 154 | 1077 | 875.5 | 665.5 | 1963 | 154 | 1963 | 770.5 |
| MT1E-ZNF641 | 1.1E−03 | 4.8E−04 | 7.1E−03 | 0.60 | 0.61 | 0.60 | 241 | 158 | 1001 | 875.5 | 665.5 | 1513 | 158 | 1513 | 770.5 |
| SERBP1-MT1E | 1.2E−03 | 5.2E−04 | 5.9E−03 | 0.60 | 0.61 | 0.60 | 262 | 171 | 887 | 875.5 | 665.5 | 1513 | 171 | 1513 | 770.5 |
| MT1E-MED6 | 1.1E−03 | 5.3E−04 | 7.7E−03 | 0.60 | 0.61 | 0.59 | 248 | 174 | 1069 | 875.5 | 665.5 | 1963 | 174 | 1963 | 770.5 |
| MT1E-SRCAP | 1.1E−03 | 5.4E−04 | 6.4E−03 | 0.60 | 0.61 | 0.59 | 260 | 180 | 938 | 875.5 | 665.5 | 1963 | 180 | 1963 | 770.5 |
| IL12RB1-MT1E | 1.3E−03 | 5.7E−04 | 7.3E−03 | 0.60 | 0.61 | 0.60 | 292 | 189 | 1036 | 875.5 | 665.5 | 1513 | 189 | 1513 | 770.5 |
| AEBP1-GINS1 | 2.8E−05 | 5.0E−05 | 5.7E−05 | 0.59 | 0.59 | 0.60 | 9 | 17 | 14 | 1526 | 1756.5 | 1582.5 | 9 | 1756.5 | 771.5 |
| KIAA1949-SNURF | 1.5E−02 | 2.1E−02 | 1.1E−02 | 0.65 | 0.65 | 0.64 | 1560 | 1976 | 1313 | 30 | 58 | 262 | 30 | 1976 | 787.5 |
| AEBP1-ANXA11 | 7.3E−03 | 2.0E−02 | 7.9E−03 | 0.63 | 0.61 | 0.62 | 978 | 1932 | 1086 | 195.5 | 571 | 599 | 195.5 | 1932 | 788.5 |
| GINS1-SNURF | 4.6E−04 | 3.5E−04 | 1.3E−03 | 0.59 | 0.60 | 0.60 | 112 | 123 | 289 | 1448 | 1306 | 1766.5 | 112 | 1766.5 | 797.5 |
| SNX27-MYCBP2 | 8.0E−03 | 1.6E−02 | 9.2E−03 | 0.63 | 0.61 | 0.62 | 1027 | 1676 | 1191 | 189.5 | 592 | 547.5 | 189.5 | 1676 | 809.5 |
| ARHGAP10-MT1E | 6.8E−04 | 4.9E−04 | 4.9E−03 | 0.60 | 0.61 | 0.59 | 158 | 162 | 755 | 875.5 | 881 | 1963 | 158 | 1963 | 815.25 |
| GPBAR1-RPS20 | 1.0E−02 | 1.2E−02 | 4.1E−03 | 0.60 | 0.62 | 0.65 | 1232 | 1445 | 658 | 1003 | 554 | 174 | 174 | 1445 | 830.5 |
| SERBP1-LYRM5 | 1.8E−03 | 1.3E−03 | 6.2E−04 | 0.59 | 0.60 | 0.60 | 384 | 353 | 156 | 1448 | 1574.5 | 1352 | 156 | 1574.5 | 868 |
| MPP6-RARS2 | 8.7E−03 | 6.8E−03 | 4.3E−04 | 0.60 | 0.61 | 0.66 | 1118 | 1052 | 110 | 1003 | 749.5 | 41 | 41 | 1118 | 876.25 |
| SELM-SIPA1L2 | 2.1E−02 | 1.5E−02 | 1.7E−02 | 0.64 | 0.64 | 0.65 | 1921 | 1667 | 1752 | 86 | 87 | 127 | 86 | 1921 | 897 |
| KIAA1949-MPP6 | 4.0E−03 | 7.7E−03 | 7.0E−04 | 0.60 | 0.59 | 0.62 | 671 | 1127 | 168 | 1283.5 | 1715.5 | 724.5 | 168 | 1715.5 | 925.75 |
| TXK-GINS1 | 5.8E−04 | 6.5E−04 | 9.9E−04 | 0.59 | 0.59 | 0.60 | 137 | 212 | 228 | 1711.5 | 1847 | 1766.5 | 137 | 1847 | 969.75 |
| PDCD6IP-MPP6 | 7.0E−03 | 1.1E−02 | 3.5E−04 | 0.59 | 0.59 | 0.61 | 945 | 1377 | 95 | 1366.5 | 1928 | 861 | 95 | 1928 | 1155.75 |
| ABHD12-NDUFC1 | 4.3E−03 | 4.6E−03 | 4.3E−04 | 0.59 | 0.59 | 0.60 | 689 | 806 | 111 | 1994 | 1847 | 1766.5 | 111 | 1994 | 1286.25 |
| MPP6-C5orf62 | 1.4E−02 | 1.4E−02 | 7.4E−04 | 0.59 | 0.59 | 0.61 | 1494 | 1569 | 178 | 1548.5 | 1715.5 | 988 | 178 | 1715.5 | 1521.25 |

TABLE 15
List of memberships of 175 SGs in 348 RGPs with high outcome
predictive performance ranks (Within the list of 348 RGP,
128 of the 175 SGs are represented, ranging from participation
of each SG in 1 to 53 different RGPs).

| SG175 ABI | SG175 PGP |
|---|---|
| Gene Symbol | count in GP348 |
| MT1E | 53 |
| SEC14L1 | 38 |
| VAMP2 | 34 |
| AEBP1 | 33 |
| GINS1 | 32 |
| NCOA4 | 21 |
| PREX1 | 17 |
| TMEM8B | 17 |
| MPP6 | 16 |
| TMEM49 | 16 |
| LYRM5 | 15 |
| ABHD12 | 14 |
| TATDN1 | 14 |
| C5orf62 | 12 |
| RPS20 | 11 |
| SELM | 11 |
| SNX27 | 11 |
| CRTAP | 10 |
| MRPL42 | 10 |
| RPL19 | 10 |
| C20orf111 | 9 |
| FOXN2 | 9 |
| CALM3 | 8 |
| PRKAR1B | 8 |
| C5orf32 | 7 |
| KLF12 | 7 |
| MAF | 7 |
| PLAC8 | 7 |
| SMARCB1 | 7 |
| SNURF | 7 |
| HMGB2 | 6 |
| KIAA1949 | 6 |
| C16orf53 | 5 |
| LAPTM5 | 5 |
| NSUN5 | 5 |
| PDCD6IP | 5 |
| SERPINB9 | 5 |
| SIPA1L2 | 5 |
| URM1 | 5 |
| XRCC1 | 5 |
| ADRB2 | 4 |
| ANXA11 | 4 |
| AP2A1 | 4 |
| C8orf33 | 4 |
| CCDC6 | 4 |
| DPP7 | 4 |
| EIF4H | 4 |
| GPBAR1 | 4 |
| MT1H | 4 |
| RPL37 | 4 |
| RPUSD1 | 4 |
| ZFAND5 | 4 |
| HEATR3 | 3 |
| MAST2 | 3 |
| NDUFC1 | 3 |
| NPTN | 3 |
| NR2F6 | 3 |
| RAB35 | 3 |
| RARS2 | 3 |
| SERBP1 | 3 |
| SMNDC1 | 3 |
| SNUPN | 3 |
| SPG7 | 3 |
| TM9SF1 | 3 |
| TMEM167B | 3 |
| UBXN4 | 3 |
| VTI1B | 3 |
| AAK1 | 2 |
| AKIRIN2 | 2 |
| CH25H | 2 |
| DGKA | 2 |
| FLT3LG | 2 |
| GCH1 | 2 |
| GRK6 | 2 |
| MPP5 | 2 |
| MT1G | 2 |
| NCDN | 2 |
| PAIP2 | 2 |
| PSMD7 | 2 |
| SARM1 | 2 |
| SEPT6 | 2 |
| SMYD3 | 2 |
| STAM | 2 |
| TIMM23 | 2 |
| TPT1 | 2 |
| YPEL5 | 2 |
| ANAPC11 | 1 |
| ARHGAP10 | 1 |
| BCL7B | 1 |
| C7orf68 | 1 |
| CBX5 | 1 |
| CC2D1B | 1 |
| CCDC130 | 1 |
| CD2BP2 | 1 |
| CDC37 | 1 |
| CRLF3 | 1 |
| CTCF | 1 |
| DGKH | 1 |
| ERMP1 | 1 |
| FGFR1OP2 | 1 |
| GCA | 1 |
| HEMK1 | 1 |
| HNRNPD | 1 |
| IKBKB | 1 |
| IL12RB1 | 1 |
| IL1RN | 1 |
| MED6 | 1 |
| MYCBP2 | 1 |
| POLR3K | 1 |
| PPHLN1 | 1 |
| PRPF3 | 1 |
| RABL2B | 1 |
| RANBP1 | 1 |
| RCHY1 | 1 |
| RNPEP | 1 |
| RPL26L1 | 1 |
| SHMT1 | 1 |
| SNRPC | 1 |
| SON | 1 |
| SRCAP | 1 |
| STRADB | 1 |
| TAF1 | 1 |
| TMEM5 | 1 |
| TTC21A | 1 |
| TXK | 1 |
| XPO1 | 1 |
| YY1AP1 | 1 |
| ZNF641 | 1 |
| ACER3 | 0 |
| ANXA7 | 0 |
| AQP3 | 0 |
| B4GALT2 | 0 |
| C10orf35 | 0 |
| CA2 | 0 |
| CCDC76 | 0 |
| CD226 | 0 |
| CD79A | 0 |
| CHST13 | 0 |
| COG5 | 0 |
| COQ5 | 0 |
| COX5A | 0 |
| CSAD | 0 |
| EI24 | 0 |
| EPS15 | 0 |
| ERH | 0 |
| FAM177A1 | 0 |
| GSTM2 | 0 |
| HBXIP | 0 |
| ICAM1 | 0 |
| KIF3B | 0 |
| N4BP2 | 0 |
| NAP1L3 | 0 |
| NCAPD2 | 0 |
| NFAM1 | 0 |
| NFATC1 | 0 |
| NUDC | 0 |
| PHAX | 0 |
| PIBF1 | 0 |
| PIGO | 0 |
| PJA2 | 0 |
| PNRC2 | 0 |
| POF1B | 0 |
| PPARG | 0 |
| RNF44 | 0 |
| SCAND2 | 0 |
| SENP7 | 0 |
| SRSF4 | 0 |
| TCN2 | 0 |
| TIMM8B | 0 |
| TLR5 | 0 |
| TMC8 | 0 |
| UTRN | 0 |
| WDR41 | 0 |
| ZFYVE19 | 0 |
| ZNF296 | 0 |

Determination of PRGPs (Pairs of RGPs):

As an alternative reduction of candidate RGPs for further refinement into a GVHD outcome prediction profile, it was conjectured that for RGPs to perform well in (operationally semi-additive) multi-RGP voting model, they should also perform well in additive pairs of RGP (PRGP, defined below) models. By preselecting RGPs that perform well in PRGPs, multi-RGP voting models may be seeded with candidate RGPs with an increased propensity to synergistically interact toward improved GVHD outcome prediction in a multi-RGP scenario.

Just as competitive interactions (see above) are expressed mathematically in a competitive, ratiometric relationship (x/y, or in logarithmic form, log(x/y), or equivalently, log x−log y), synergistic interactions are expressed mathematically in a synergistic, multiplicative relationship (x*y, or in logarithmic form, log (x*y), or equivalently, log x+log y). In the technical implementation described herein, because the RRCF and RGP values are expressed in logarithmic form of underlying mRNA concentration measurement levels, such synergistic interactions would be expressed in additive form with respect to RGP values.

PRGP values for RGP/RRCF data are defined as follows:

PRGP=RGP X+RGP Y.

When reduced to SG measurements, at the RRCF level (RL2F calibrated by HSK signal subtraction), PRGPs are defined as, for

RGP X=RRCF A−RRCF B, and for

RGP Y=RRCF C−RRCF D, as

PRGP=(RRCF A−RRCF B)+(RRCF C−RRCF D)

Alternatively, when reduced to SG measurements, at the RL2F level, PRGPs are defined as, for

RGP X=RL2F A−RL2F B, and for

RGP Y=RL2F C−RL2F D, as

PRGP=(RL2F A−RL2F B)+(RL2F C−RL2F D)

For PRGP determinations, 175 RGPs were selected from the RGP348 list (Table 14) according to the following criteria:
(1) Only consider RGPs for which both gene members that show a minimal performance rank of 100, i.e. filter for minimal rank<=100
(2) Prioritize filtered RGPs according to median rank and select the best 175 median ranking RGPs

All of the selected 175 RGPs for PRGP determination show a minimal rank<=100, median rank<=464, and maximal rank<=1380.

The PRGP values for all 180 samples were determined for the complete set 15,225 unique PRGPs from the derived select 175 RGPs (analogously as described above for RGPs). GVHD outcome predictive performance and rankings were evaluated for the set of 15,225 PRGPs analogously as described above for RGPs.

Evaluation of Outcome Predictive Performance of PRGPs:

A set of 348 PRGPs (PRGP348; Table 16 lists the specific PRGPs) was selected from the complete set of 15,225 PRGPs by
(1) requiring each PRGP to have over all 6 predictive performance variable ranks a maximal rank (from 1 to 15,225)<=5000, and minimal rank<=500, resulting in 890 PRGPs,
(2) prioritizing within this set of 890 PRGPs the best 348 median ranking PRGPs.

Performance values and rankings were determined for PRGPs analogously as described above for RGPs

TABLE 16
List of 348 PRGPs (PRGP348)

| PRGP ABI Gene Symbol | PRGP ABI Gene Symbol | PRGP ABI Gene Symbol |
|---|---|---|
| TMEM49-VAMP2-ABHD12-CALM3 | AEBP1-RPUSD1-VAMP2-URM1 | PREX1-SMARCB1-GINS1-KLF12 |
| AEBP1-NCDN-ANAPC11-GINS1 | SEC14L1-LYRM5-GINS1-CALM3 | TMEM49-TATDN1-GINS1-MT1H |
| VAMP2-GCH1-LYRM5-TPT1 | SEC14L1-CALM3-GINS1-LYRM5 | MT1E-GINS1-AEBP1-DGKA |
| VAMP2-C5orf32-CALM3-HEATR3 | ANAPC11-GINS1-MRPL42-HEATR3 | AEBP1-ZFAND5-SELM-GINS1 |
| ANAPC11-GINS1-TATDN1-C5orf62 | VAMP2-SEC14L1-AEBP1-ZFAND5 | MAF-LYRM5-NCOA4-RPS20 |
| ABHD12-MPP6-TMEM49-VAMP2 | AEBP1-SEC14L1-VAMP2-ZFAND5 | PREX1-MPP6-GINS1-CALM3 |
| AEBP1-SEC14L1-ANAPC11-GINS1 | MAF-RPL19-NCOA4-RPS20 | VAMP2-CCDC6-AEBP1-ABHD12 |
| VAMP2-GINS1-AEBP1-SPG7 | ANAPC11-GINS1-VAMP2-C5orf62 | GINS1-MT1H-ADRB2-AEBP1 |
| ANAPC11-GINS1-AEBP1-SNX27 | AEBP1-NCDN-MPP5-SEC14L1 | ANAPC11-GINS1-TMEM8B-NSUN5 |
| AAK1-SEC14L1-TMEM8B-C16orf53 | MAF-RPS20-TMEM49-AEBP1 | PDCD6IP-LYRM5-TMEM49-CALM3 |
| ANAPC11-GINS1-AEBP1-NCOA4 | TMEM49-TATDN1-MAF-RPL19 | VAMP2-TMEM5-MRPL42-CCDC6 |
| VAMP2-CRTAP-ANAPC11-GINS1 | C5orf62-RPL19-GINS1-CALM3 | TMEM49-VAMP2-SEC14L1-PAIP2 |
| VAMP2-LAPTM5-XRCC1-PREX1 | VAMP2-URM1-AEBP1-DGKA | SELM-RPUSD1-SEPT6-GINS1 |
| VAMP2-NCOA4-ANAPC11-GINS1 | MAF-RPS20-TMEM49-SNUPN | RPL37-GINS1-AEBP1-SPG7 |
| VAMP2-LAPTM5-AEBP1-SARM1 | VAMP2-TMEM5-PLAC8-SEC14L1 | TMEM49-TATDN1-NCOA4-PLAC8 |
| VAMP2-PREX1-TMEM8B-SEC14L1 | VAMP2-TMEM5-TATDN1-SEC14L1 | TMEM49-PLAC8-NCOA4-TATDN1 |
| TMEM49-TATDN1-PREX1-SMARCB1 | TMEM49-TATDN1-TMEM49-CALM3 | ANAPC11-GINS1-XRCC1-SEC14L1 |
| ANAPC11-GINS1-AEBP1-ZFAND5 | MAF-RPS20-NCOA4-MPP6 | MPP5-SEC14L1-VAMP2-SERBP1 |
| MAF-RPS20-TMEM49-CALM3 | VAMP2-ZFAND5-RABL2B-GINS1 | VAMP2-NCOA4-MPP5-SEC14L1 |
| ANAPC11-GINS1-VAMP2-KIAA1949 | AEBP1-RPUSD1-LYRM5-TPT1 | AEBP1-ZFAND5-SMARCB1-GINS1 |
| TMEM49-VAMP2-MAF-RPS20 | VAMP2-C5orf62-SMARCB1-GINS1 | PDCD6IP-TATDN1-TMEM49-CALM3 |
| VAMP2-SEC14L1-TMEM8B-NSUN5 | MAF-RPS20-TMEM49-MPP6 | VAMP2-TMEM5-SMARCB1-SEC14L1 |
| SELM-NCOA4-TMEM8B-C16orf53 | AEBP1-RPUSD1-VAMP2-GCH1 | ADRB2-AEBP1-MAST2-MT1E |
| ABHD12-MPP6-TMEM49-CALM3 | VAMP2-NCOA4-SMARCB1-GINS1 | TMEM49-PLAC8-CRTAP-CALM3 |
| TMEM49-MPP6-ABHD12-CALM3 | AEBP1-NCOA4-PLAC8-GINS1 | CRTAP-PLAC8-TMEM49-CALM3 |
| VAMP2-C5orf32-VAMP2-TMEM5 | TMEM49-VAMP2-ZFAND5-C20orf111 | VAMP2-SEC14L1-XRCC1-GINS1 |
| MPP5-SEC14L1-TMEM8B-C16orf53 | VAMP2-RARS2-TMEM8B-SEC14L1 | XRCC1-SEC14L1-VAMP2-GINS1 |
| GINS1-CALM3-NCOA4-MRPL42 | PDCD6IP-LYRM5-SEC14L1-CALM3 | TMEM49-TATDN1-NCOA4-C8orf33 |
| ANAPC11-GINS1-PLAC8-SEC14L1 | AEBP1-SEC14L1-VAMP2-TMEM5 | PREX1-C20orf111-GINS1-KLF12 |
| ANAPC11-GINS1-TMEM8B-C5orf62 | VAMP2-C5orf32-VAMP2-GINS1 | NCOA4-MPP6-PREX1-C20orf111 |
| SELM-RPUSD1-AEBP1-NSUN5 | AEBP1-NCDN-VAMP2-NCOA4 | PREX1-MPP6-NCOA4-C20orf111 |
| AEBP1-ZFAND5-VAMP2-TMEM5 | AEBP1-NCDN-VAMP2-ZFAND5 | ABHD12-MPP6-PREX1-C20orf111 |
| ANAPC11-GINS1-AEBP1-NSUN5 | TMEM49-TATDN1-TMEM49-ANXA11 | VAMP2-TMEM5-AEBP1-SPG7 |
| NCOA4-TATDN1-GINS1-CALM3 | VAMP2-SEC14L1-ANAPC11-GINS1 | AEBP1-SEC14L1-TATDN1-GINS1 |
| VAMP2-SEC14L1-TMEM8B-C16orf53 | XRCC1-PREX1-VAMP2-TMEM5 | VAMP2-PREX1-PLAC8-GINS1 |
| MAF-RPS20-TMEM49-LYRM5 | ANAPC11-GINS1-TATDN1-SEC14L1 | TMEM8B-C5orf62-RABL2B-GINS1 |
| AEBP1-RPUSD1-VAMP2-ZFAND5 | VAMP2-CCDC6-HMGB2-C5orf32 | VAMP2-SEC14L1-SEPT6-GINS1 |
| VAMP2-SEC14L1-AEBP1-SARM1 | NCOA4-MPP6-TMEM49-CALM3 | AEBP1-NCDN-VAMP2-SERBP1 |
| ANAPC11-GINS1-VAMP2-GCH1 | AEBP1-SARM1-RABL2B-GINS1 | MRPL42-GINS1-AEBP1-ZFAND5 |
| ANAPC11-GINS1-SELM-NCOA4 | TMEM49-TATDN1-ABHD12-CALM3 | TMEM49-AEBP1-NCOA4-TATDN1 |
| SEC14L1-LYRM5-TMEM49-CALM3 | VAMP2-SEC14L1-AEBP1-RPUSD1 | C8orf33-SEC14L1-VAMP2-TMEM5 |
| SEC14L1-CALM3-TMEM49-LYRM5 | AEBP1-RPUSD1-VAMP2-ABHD12 | AEBP1-RPUSD1-HMGB2-SEC14L1 |
| TMEM8B-SEC14L1-MRPL42-FOXN2 | AEBP1-RPUSD1-VAMP2-TMEM5 | LYRM5-TPT1-RABL2B-GINS1 |
| ANAPC11-GINS1-VAMP2-C5orf32 | MT1E-GINS1-AEBP1-RPUSD1 | SNX27-TATDN1-GINS1-CALM3 |
| VAMP2-URM1-MRPL42-CCDC6 | VAMP2-TMEM5-HMGB2-C5orf32 | AEBP1-SNX27-VAMP2-SERBP1 |
| AEBP1-RPUSD1-RABL2B-GINS1 | GINS1-LYRM5-NCOA4-C20orf111 | VAMP2-TMEM5-AEBP1-NCOA4 |
| AEBP1-RPUSD1-VAMP2-GINS1 | TMEM49-VAMP2-PREX1-SNURF | VAMP2-ZFAND5-AEBP1-GRK6 |
| AEBP1-SPG7-TMEM8B-NSUN5 | AEBP1-NSUN5-RABL2B-GINS1 | TMEM49-TATDN1-PREX1-KLF12 |
| TMEM49-CALM3-ABHD12-RPL19 | VAMP2-KIAA1949-AEBP1-SARM1 | VAMP2-SEC14L1-AEBP1-NSUN5 |
| TMEM49-VAMP2-PREX1-MPP6 | ANAPC11-GINS1-HMGB2-C5orf32 | VAMP2-NCOA4-AEBP1-DGKA |
| AEBP1-RPUSD1-VAMP2-NCOA4 | C5orf62-RPS20-GINS1-CALM3 | MPP6-SEC14L1-VAMP2-ABHD12 |
| MPP6-SEC14L1-VAMP2-SERBP1 | VAMP2-ZFAND5-AEBP1-DGKA | MPP6-SEC14L1-AEBP1-NSUN5 |
| AEBP1-SPG7-XRCC1-GINS1 | ANAPC11-GINS1-AAK1-SEC14L1 | VAMP2-PREX1-AEBP1-SNX27 |
| PREX1-SMARCB1-GINS1-CALM3 | AEBP1-ZFAND5-VAMP2-URM1 | VAMP2-LAPTM5-AEBP1-ZFAND5 |
| AEBP1-RPUSD1-SELM-NCOA4 | VAMP2-NCOA4-AEBP1-ZFAND5 | VAMP2-TMEM5-AEBP1-GRK6 |
| SELM-RPUSD1-AEBP1-NCOA4 | VAMP2-ZFAND5-AEBP1-NCOA4 | GINS1-PRKAR1B-PREX1-SMARCB1 |
| AEBP1-NCDN-VAMP2-C5orf62 | GINS1-CALM3-PREX1-MRPL42 | ANAPC11-GINS1-HMGB2-SEC14L1 |
| NCOA4-PLAC8-GINS1-CALM3 | VAMP2-CRTAP-AEBP1-RPUSD1 | TMEM49-CALM3-NSUN5-MPP6 |
| VAMP2-PREX1-TMEM8B-C5orf62 | NCOA4-C8orf33-PDCD6IP-LYRM5 | MRPL42-FOXN2-RABL2B-GINS1 |
| MRPL42-GINS1-AEBP1-SPG7 | VAMP2-ZFAND5-AEBP1-ABHD12 | VAMP2-C5orf32-VAMP2-ABHD12 |
| VAMP2-NCOA4-TMEM8B-SEC14L1 | AEBP1-ZFAND5-VAMP2-ABHD12 | SEC14L1-SNURF-GINS1-CALM3 |
| TMEM49-TATDN1-TMEM49-FLT3LG | TMEM49-TATDN1-TMEM49-PLAC8 | SELM-RPUSD1-AEBP1-DGKA |
| TMEM49-TATDN1-GINS1-CALM3 | VAMP2-URM1-AEBP1-SPG7 | NCOA4-C8orf33-MAF-RPS20 |
| ANAPC11-GINS1-VAMP2-ZFAND5 | VAMP2-URM1-AEBP1-GRK6 | ANAPC11-GINS1-SMARCB1-SEC14L1 |
| C20orf111-SEC14L1-AEBP1-NCDN | VAMP2-C5orf32-TMEM8B-NSUN5 | MPP5-SEC14L1-VAMP2-URM1 |
| PREX1-SNURF-GINS1-CALM3 | XRCC1-PREX1-RABL2B-GINS1 | MT1E-C5orf32-PLAC8-GINS1 |
| AEBP1-ZFAND5-VAMP2-GINS1 | VAMP2-LAPTM5-AEBP1-NCDN | TMEM49-MRPL42-MAF-RPS20 |
| VAMP2-ABHD12-TMEM8B-C16orf53 | AEBP1-NCDN-MRPL42-FOXN2 | GINS1-PRKAR1B-SEC14L1-CALM3 |
| XRCC1-PREX1-TMEM8B-C5orf62 | VAMP2-PREX1-SELM-GINS1 | SEC14L1-PRKAR1B-GINS1-CALM3 |
| TMEM49-PLAC8-GINS1-CALM3 | MAF-LYRM5-GINS1-LYRM5 | NCOA4-PLAC8-MAF-RPS20 |
| ANAPC11-GINS1-VAMP2-ABHD12 | MT1E-GINS1-AEBP1-GRK6 | TMEM49-CALM3-MAF-RPL19 |
| VAMP2-ZFAND5-AEBP1-SNX27 | MT1E-GINS1-AEBP1-SNX27 | C20orf111-SEC14L1-VAMP2-ABHD12 |
| ANAPC11-GINS1-VAMP2-PREX1 | VAMP2-NCOA4-VAMP2-GINS1 | VAMP2-GCH1-PLAC8-GINS1 |
| TMEM49-LYRM5-ABHD12-CALM3 | NCOA4-TATDN1-TMEM49-CALM3 | NCOA4-SNURF-GINS1-CALM3 |
| ANAPC11-GINS1-SELM-C5orf62 | AEBP1-NCDN-SELM-NCOA4 | VAMP2-KIAA1949-PLAC8-GINS1 |
| TMEM49-VAMP2-FOXN2-MPP6 | MAF-LYRM5-FOXN2-RPS20 | ANAPC11-GINS1-VAMP2-AKIRIN2 |
| VAMP2-GINS1-AEBP1-NSUN5 | ANAPC11-GINS1-C8orf33-SEC14L1 | VAMP2-PREX1-AEBP1-NSUN5 |
| MAF-RPS20-TMEM49-ANXA11 | AEBP1-NCDN-VAMP2-ABHD12 | MPP5-SEC14L1-VAMP2-ABHD12 |
| MT1E-GINS1-AEBP1-NCDN | MT1E-GINS1-AEBP1-NSUN5 | AEBP1-RPUSD1-VAMP2-RARS2 |
| AEBP1-SPG7-PLAC8-GINS1 | ANAPC11-GINS1-LYRM5-GPBAR1 | VAMP2-NCOA4-LYRM5-TPT1 |
| PREX1-SNURF-TMEM49-CALM3 | ANAPC11-GINS1-HMGB2-C5orf62 | GINS1-PRKAR1B-ABHD12-CALM3 |
| XRCC1-PREX1-VAMP2-ABHD12 | VAMP2-SEC14L1-VAMP2-TMEM5 | AEBP1-NCDN-SELM-GINS1 |
| AEBP1-NCDN-SMARCB1-SEC14L1 | TMEM49-CALM3-GINS1-RPS20 | TMEM49-TATDN1-NCOA4-C20orf111 |
| GINS1-CALM3-NCOA4-RPS20 | XRCC1-PREX1-SELM-GINS1 | TMEM49-MT1E-GINS1-RPS20 |
| ANAPC11-GINS1-AEBP1-ABHD12 | TMEM49-TATDN1-CRTAP-CALM3 | MPP6-SEC14L1-VAMP2-URM1 |
| MT1E-GINS1-AEBP1-SARM1 | VAMP2-C5orf62-TMEM8B-C16orf53 | FOXN2-RPS20-TMEM49-CALM3 |
| SEPT6-GINS1-AEBP1-SPG7 | VAMP2-SEC14L1-TMEM8B-SEC14L1 | VAMP2-URM1-AEBP1-NSUN5 |
| MRPL42-GINS1-AEBP1-NSUN5 | VAMP2-KIAA1949-AEBP1-GRK6 | SEC14L1-CALM3-GINS1-RPS20 |
| MPP6-SEC14L1-VAMP2-TMEM5 | TMEM8B-C5orf62-VAMP2-AP2A1 | SEC14L1-RPS20-GINS1-CALM3 |
| VAMP2-ZFAND5-AEBP1-NSUN5 | VAMP2-CRTAP-AEBP1-SPG7 | ABHD12-MRPL42-GINS1-CALM3 |
| SEC14L1-CALM3-TMEM49-MPP6 | MT1E-GINS1-AEBP1-TM9SF1 | TMEM49-VAMP2-SEC14L1-PRKAR1B |
| ANAPC11-GINS1-TMEM8B-SEC14L1 | VAMP2-PREX1-STRADB-GINS1 | VAMP2-SEC14L1-STRADB-GINS1 |
| MT1E-GINS1-AEBP1-ZFAND5 | VAMP2-PREX1-AEBP1-DGKA | C20orf111-SEC14L1-VAMP2-GCH1 |
| MRPL42-GINS1-AEBP1-NCOA4 | VAMP2-ABHD12-CALM3-HEATR3 | AEBP1-NCDN-HMGB2-C5orf32 |
| TMEM49-TATDN1-MAF-RPS20 | GINS1-LYRM5-NSUN5-KLF12 | VAMP2-ABHD12-AEBP1-NSUN5 |
| XRCC1-SEC14L1-VAMP2-TMEM5 | PREX1-AAK1-GINS1-CALM3 | GINS1-MT1H-TMEM49-AEBP1 |
| ZFAND5-C20orf111-TMEM49-CALM3 | AEBP1-NSUN5-PLAC8-GINS1 | VAMP2-C5orf32-TMEM8B-SEC14L1 |
| PREX1-KLF12-GINS1-CALM3 | AEBP1-NCDN-HMGB2-SEC14L1 | VAMP2-ABHD12-AEBP1-GRK6 |
| MRPL42-GINS1-VAMP2-PREX1 | VAMP2-NCOA4-STRADB-GINS1 | TMEM49-FLT3LG-PDCD6IP-TATDN1 |
| AEBP1-SPG7-TMEM8B-SEC14L1 | XRCC1-PREX1-PLAC8-GINS1 | VAMP2-ABHD12-MRPL42-CCDC6 |
| VAMP2-KIAA1949-AEBP1-ZFAND5 | XRCC1-PREX1-SEPT6-GINS1 | AEBP1-NCDN-PLAC8-GINS1 |
| ANAPC11-GINS1-TMEM8B-C16orf53 | ANAPC11-GINS1-RPL37-C5orf62 | MAF-RPS20-NCOA4-PAIP2 |
| AEBP1-NCDN-MPP6-SEC14L1 | VAMP2-LAPTM5-AEBP1-SPG7 | GINS1-RPL19-ABHD12-CALM3 |
| AEBP1-RPUSD1-SELM-GINS1 | NCOA4-C8orf33-NSUN5-MPP6 | GINS1-CALM3-ABHD12-RPL19 |
| ANAPC11-GINS1-VAMP2-URM1 | VAMP2-LAPTM5-AEBP1-DGKA | TMEM49-VAMP2-CRTAP-PLAC8 |
| VAMP2-PREX1-SMARCB1-GINS1 | TMEM49-PLAC8-NCOA4-MPP6 | TMEM49-SNUPN-MAF-RPL19 |
| AEBP1-SPG7-TATDN1-GINS1 | NCOA4-PLAC8-TMEM49-MPP6 | TMEM49-VAMP2-PREX1-SMARCB1 |
| TMEM49-VAMP2-SEC14L1-CALM3 | AEBP1-RPUSD1-VAMP2-PREX1 | GINS1-MT1H-C5orf62-RPS20 |
| XRCC1-PREX1-VAMP2-GINS1 | VAMP2-SEC14L1-AEBP1-DGKA | AEBP1-GRK6-SMARCB1-GINS1 |
| VAMP2-PREX1-XRCC1-GINS1 | GINS1-PRKAR1B-ADRB2-MT1E | TMEM49-AEBP1-PREX1-SMARCB1 |
| NR2F6-PRKAR1B-GINS1-CALM3 | AEBP1-NCDN-XRCC1-SEC14L1 | VAMP2-ABHD12-AEBP1-DGKA |
| MT1E-GINS1-AEBP1-SPG7 | VAMP2-NCOA4-AEBP1-SPG7 | ABHD12-CALM3-GINS1-RPS20 |
| VAMP2-ZFAND5-AEBP1-SARM1 | AEBP1-RPUSD1-VAMP2-SERBP1 | ADRB2-AEBP1-GINS1-LYRM5 |
| ANAPC11-GINS1-MRPL42-SEC14L1 | TMEM49-VAMP2-SEC14L1-CRLF3 | AEBP1-TM9SF1-VAMP2-ABHD12 |
| ANAPC11-GINS1-AEBP1-SARM1 | C20orf111-SEC14L1-VAMP2-TMEM5 | TMEM49-TATDN1-GINS1-PRKAR1B |
| AEBP1-NCDN-VAMP2-C5orf32 | MT1E-GINS1-AEBP1-ABHD12 | VAMP2-NCOA4-AEBP1-GRK6 |

### Example 13

This example includes a description of alternative RGP Vmod (voting model) implementations of the GVHD prediction, definition of top-performing RGP Vmods, and other well-performing alternative RGP Vmods.

In addition to harnessing the combined ratiometric GVHD outcome predictive and self-calibrating properties of the RGPs selected above, further accuracy and robustness in GVHD outcome prediction would be expected to be achieved by averaging out errors contributed by individual RGP voters through the use of multi-RGP voting models (Vmods). Within such a GVHD outcome prediction Vmod, prioritized subsets of RGPs are used to provide individual “N” (N=not causing GVHD in the recipient) outcome predictive votes, and these votes are aggregated and averaged as a GVHD N Outcome Score, or GNOS. In turn, when the GNOS is above a pre-determined “GNOS threshold” level, a donor sample is ultimately called as N, or “likely to lead to a GVHD NEGATIVE outcome in the recipient when used for transplantation.”

Selection of Alternative RGP Vmods:

Multiple, principled ways have been applied for aggregating the RGPs (or PRGPs, for indirect RGP selection) into Vmods for GNOS determination, such as to result in GVHD outcome prediction using a total of 48 SGs, including the 6 HSKs listed above (Table 12) for initial SG calibration. The list of RGPs and SGs contributing to the different Vmods is detailed in below (see Tables 17 and 18, VmodRGP100 and VmodSG64, respectively).

Note that Vmod GNOS calculations for all Vmods are always directly carried out on RGPs values, and never directly on PRGP or SG values (even though PRGPs have contributed to RGP selections, and SG values are used to determine the RGP values).

Three basic methods are outlined below for RGP prioritization from the RGP348 list (Table 14) into 3 alternative Vmods:
(1) Vmod: SG43RGP46-GPperformance:
   RGPs in RGP348 list (Table 14) were prioritized solely according to median performance rank, without any restriction of contributing SGs being a member of multiple GPs. The top median performance ranking 46 RGPs contain 43 unique SGs, including one of the HSK6 SGs (Table 12). Combining these 43 SGs with the remaining 5 HSK6 SGs, results in a total of 48 SGs for implementation as the GVHD outcome prediction test.
(2) Vmod: SG42RGP21-GPminimalist
   RGPs in RGP348 list (Table 14) were prioritized according to median performance rank. After the best ranking RGP is selected to go into the Vmod, all RGPs containing SGs already selected for the Vmod are removed from the candidate list, and then the next best ranking RGP is selected. The top 21 ranking RGPs, not allowing for any SG to appear in more than one RGP, result in a total of 42 unique SGs. Combining these 42 SGs with the remaining 6 HSK6 SGs results in a total of 48 SGs for implementation as a GVHD outcome prediction test.
(3) Vmod: SG43RGP37-GPconnectivity:
   The 121 SGs contributing to the RGP348 list (Table 14) were prioritized in two steps, i.e.,
      1. the highest numbers of RGPs for which a particular SG is a member, i.e., the highest SG connectivities in the RGP network, and
      2. the best median outcome predictive performance rank (over the 6 standard performance ranks, see above).
   The 43 combined top rankings SGs were selected according to this method. RGPs, including only SG members from this list of 43 SGs, where then prioritized according to median performance rank. The minimal number of top ranking RGPs from this restricted selection for which each of the select 43 SGs appears in at least once, was then selected by aggregating the prioritized RGPs in median performance rank order; however, only allowing new RGPs in the aggregation process to be included if none or only one (not both) of its SG members is found in the RGPs already selected for the Vmod. (Note: Without this restriction, likely more than 100 GPs would be required to cover the 43 most connected SGs, because many of the most connected SGs participate in lower ranking RGPs). A total number of 37 RGPs contribute to this Vmod, containing 43 unique SGs, including one of the HSK6 SGs. Combining these 43 SGs with the remaining 5 HSK6 SGs, results in a total of 48 SGs for implementation as a GVHD outcome prediction test.

By preselecting RGPs that perform well in PRGPs, Vmods may be seeded with candidate RGPs with an increased propensity to synergistically interact toward improved outcome prediction in a multi-RGP scenario. Two basic methods are outlined below for PRGP (and implicit contributing RGP and SG) prioritization from the PRGP348 list (Table 16) into 2 alternative Vmods:
(1) Vmod: SG43RGP51−PRGPminranksort:
   PRGPs in the PRGP348 list (see Table 16) were prioritized first by maximal (worst) performance rank, then by median rank, and then by minimal (best) rank, such that the final prioritization criterion is the best, i.e., minimal, performance rank. No restrictions were placed on SGs or RGPs being a member of multiple PRGPs. The top median performance ranking 45 PRGPs contain 51 unique RGPs, and 43 unique SGs, including one of the HSK6 SGs. Combining these 43 SGs with the remaining 5 HSK6 SGs, results in a total of 48 SGs for implementation as a GVHD outcome prediction test.
(2) Vmod: SG43RGP55-PRGPmedranksort:
   PRGPs in the PRGP348 list (Table 16) were prioritized first by maximal (worst) performance rank, then by minimal (best) rank, and then by median rank, such that the final prioritization criterion is the median performance rank. No restrictions were placed on SGs or RGPs being a member of multiple PRGPs. The top median performance ranking 60 PRGPs contain 55 unique RGPs, and 43 unique SGs, including one of the HSK6 SGs. Combining these 43 SGs with the remaining 5 HSK6 SGs, results in a total of 48 SGs for implementation as a GVHD outcome prediction test.

The set of 5 Vmods described above contain a total of 100 unique RGPs, and 64 unique SGs, which are listed in Tables 17 and 18 below, VmodRGP100 and VmodSG64, respectively.

TABLE 17
Vmod memberships of 100 RGPs (“VmodRGP100”) that participate in the alternative Vmod GVHD outcome prediction implementation

| | | | | SG43RGP51- | SG43RGP55- | SG43RGP36- | SG21RGP28- |
|---|---|---|---|---|---|---|---|
| RGP ABI | SG43RGP46- | SG42RGP21- | SG43RGP37- | PRGPmin- | PRGPmed- | RGPgreedy- | RGPmaxgreedy- |
| Gene Symbol | RGPperformance | RGPminimalist | RGPconnectivity | ranksort | ranksort | search | search |
| AEBP1-SEC14L1 | x | — | x | x | x | x | x |
| AEBP1-NCDN | x | — | — | x | x | x | x |
| ANAPC11-GINS1 | x | — | — | x | x | x | x |
| MT1E-GINS1 | x | — | x | — | x | x | x |
| PDCD6IP-LYRM5 | x | x | x | — | — | x | x |
| MPP5-SEC14L1 | x | — | — | x | — | x | x |
| MRPL42-GINS1 | x | — | — | — | x | x | x |
| PDCD6IP-TATDN1 | x | — | — | — | — | x | x |
| RPL37-GINS1 | x | — | — | — | — | x | x |
| TMEM8B-C16orf53 | — | — | x | x | x | x | x |
| TMEM8B-C5orf62 | — | — | x | x | x | x | x |
| ABHD12-CALM3 | — | — | — | x | x | x | x |
| AEBP1-SARM1 | — | — | — | x | x | x | x |
| PREX1-SMARCB1 | — | — | — | x | x | x | x |
| TMEM49-CALM3 | — | — | — | x | x | x | x |
| VAMP2-TMEM5 | — | — | — | x | x | x | x |
| CALM3-HEATR3 | — | — | — | x | — | x | x |
| MRPL42-FOXN2 | — | — | — | x | — | x | x |
| PLAC8-SEC14L1 | — | — | — | x | — | x | x |
| TMEM8B-NSUN5 | — | — | — | x | — | x | x |
| TMEM8B-SIPA1L2 | — | — | x | — | — | x | x |
| AEBP1-RPUSD1 | x | x | x | x | x | x | — |
| TMEM49-TATDN1 | x | x | x | x | x | x | — |
| ADRB2-MT1E | x | x | x | — | — | x | — |
| GINS1-MT1H | x | x | — | — | — | x | — |
| TMEM49-FLT3LG | x | — | — | — | x | x | — |
| TMEM49-MRPL42 | x | — | x | — | — | x | — |
| NCOA4-PAIP2 | x | — | — | — | — | x | — |
| AEBP1-ZFAND5 | — | — | — | x | x | x | — |
| FOXN2-SNURF | — | x | — | — | — | x | — |
| MRPL42-CCDC6 | — | — | — | x | — | x | — |
| NR2F6-PRKAR1B | — | x | — | — | — | x | — |
| TMEM8B-TM9SF1 | — | x | — | — | — | x | — |
| VAMP2-FOXN2 | — | — | x | — | — | x | — |
| VAMP2-SERPINB9 | — | — | x | — | — | x | — |
| XRCC1-SNX27 | — | x | — | — | — | x | — |
| MAF-RPS20 | x | x | x | x | x | — | — |
| VAMP2-SEC14L1 | x | x | x | x | x | — | — |
| ABHD12-MPP6 | x | x | x | x | — | — | — |
| SEC14L1-LYRM5 | x | — | x | x | x | — | — |
| SELM-RPUSD1 | x | — | x | x | x | — | — |
| VAMP2-C5orf32 | x | — | x | x | x | — | — |
| VAMP2-CRTAP | x | — | x | x | x | — | — |
| VAMP2-KIAA1949 | x | — | x | x | x | — | — |
| VAMP2-NCOA4 | x | — | x | x | x | — | — |
| VAMP2-ZFAND5 | x | — | x | x | x | — | — |
| AAK1-SEC14L1 | x | — | — | x | x | — | — |
| PREX1-KLF12 | x | x | x | — | — | — | — |
| SELM-NCOA4 | x | — | — | x | x | — | — |
| TMEM49-VAMP2 | x | — | — | x | x | — | — |
| VAMP2-C5orf62 | x | — | x | — | x | — | — |
| VAMP2-LAPTM5 | x | — | x | x | — | — | — |
| XRCC1-PREX1 | x | — | x | x | — | — | — |
| AEBP1-TM9SF1 | x | — | — | — | x | — | — |
| C20orf111-SEC14L1 | x | — | x | — | — | — | — |
| CRTAP-PLAC8 | x | x | — | — | — | — | — |
| GINS1-PRKAR1B | x | — | x | — | — | — | — |
| NCOA4-C8orf33 | x | x | — | — | — | — | — |
| NCOA4-MPP6 | x | — | — | — | x | — | — |
| NCOA4-PLAC8 | x | — | — | — | x | — | — |
| SEC14L1-RPL19 | x | — | x | — | — | — | — |
| SNX27-TATDN1 | x | — | x | — | — | — | — |
| TMEM49-PLAC8 | x | — | x | — | — | — | — |
| VAMP2-GCH1 | x | — | — | x | — | — | — |
| CRTAP-LYRM5 | x | — | — | — | — | — | — |
| MPP6-SEC14L1 | x | — | — | — | — | — | — |
| AEBP1-NSUN5 | — | — | x | x | x | — | — |
| SEC14L1-CALM3 | — | — | x | x | x | — | — |
| VAMP2-URM1 | — | — | x | x | x | — | — |
| AEBP1-NCOA4 | — | — | — | x | x | — | — |
| AEBP1-SNX27 | — | — | — | x | x | — | — |
| AEBP1-SPG7 | — | — | — | x | x | — | — |
| C5orf62-RPL19 | — | x | — | — | x | — | — |
| GINS1-CALM3 | — | — | — | x | x | — | — |
| MRPL42-HEATR3 | — | x | — | — | x | — | — |
| NCOA4-MRPL42 | — | — | — | x | x | — | — |
| NCOA4-TATDN1 | — | — | — | x | x | — | — |
| TMEM8B-SEC14L1 | — | — | — | x | x | — | — |
| VAMP2-GINS1 | — | — | — | x | x | — | — |
| VAMP2-PREX1 | — | — | — | x | x | — | — |
| AEBP1-ABHD12 | — | — | — | — | x | — | — |
| C5orf62-RPS20 | — | — | — | — | x | — | — |
| FOXN2-MPP6 | — | — | — | — | x | — | — |
| GINS1-LYRM5 | — | — | — | — | x | — | — |
| HMGB2-C5orf32 | — | x | — | — | — | — | — |
| HMGB2-SEC14L1 | — | — | x | — | — | — | — |
| IL1RN-MT1G | — | x | — | — | — | — | — |
| LYRM5-TPT1 | — | — | — | x | — | — | — |
| NCOA4-C20orf111 | — | — | — | — | x | — | — |
| PREX1-AAK1 | — | — | — | — | x | — | — |
| PREX1-SNURF | — | — | — | — | x | — | — |
| SEC14L1-SNURF | — | — | x | — | — | — | — |
| SELM-AP2A1 | — | x | — | — | — | — | — |
| SELM-C5orf62 | — | — | — | — | x | — | — |
| SMARCB1-SEC14L1 | — | — | x | — | — | — | — |
| TATDN1-C5orf62 | — | — | — | x | — | — | — |
| TMEM49-LYRM5 | — | — | — | x | — | — | — |
| TMEM49-MPP6 | — | — | — | x | — | — | — |
| VAMP2-ABHD12 | — | — | — | — | x | — | — |
| ZFAND5-C20orf111 | — | x | — | — | — | — | — |

(An “x” in a column indicates that the RGP in the corresponding row is a member of the Vmod listed in the column; otherwise the “—” indicates the RGP is not a component of the Vmod)

TABLE 18
Vmod memberships of 64 SGs (“VmodSG64”) that participate in the alternative Vmod GVHD outcome prediction implementation

| | | | | SGs from | SGs from | | |
|---|---|---|---|---|---|---|---|
| SG ABI | SGs from | SGs from | SGs from | SG43RGP51- | SG43RGP55- | SGs from | SGs from |
| Gene | SG43RGP46- | SG42RGP21- | SG43RGP37- | PRGPmin- | PRGPmed- | SG43RGP36- | SG21RGP28- |
| Symbol | RGPperformance | RGPminimalist | RGPconnectivity | ranksort | ranksort | RGPgreedysearch | RGPmaxgreedysearch |
| VAMP2 | x | x | x | x | x | x | x |
| SEC14L1 | x | x | x | x | x | x | x |
| AEBP1 | x | x | x | x | x | x | x |
| GINS1 | x | x | x | x | x | x | x |
| TMEM49 | x | x | x | x | x | x | x |
| MRPL42 | x | x | x | x | x | x | x |
| TATDN1 | x | x | x | x | x | x | x |
| PREX1 | x | x | x | x | x | x | x |
| LYRM5 | x | x | x | x | x | x | x |
| C5orf62 | x | x | x | x | x | x | x |
| ABHD12 | x | x | x | x | x | x | x |
| PLAC8 | x | x | x | x | x | x | x |
| MT1E | x | x | x | — | x | x | x |
| PDCD6IP | x | x | x | — | — | x | x |
| ANAPC11 | x | — | — | x | x | x | x |
| NCDN | x | — | — | x | x | x | x |
| MPP5 | x | — | — | x | — | x | x |
| RPL37 | x | — | — | — | — | x | x |
| TMEM8B | — | x | x | x | x | x | x |
| FOXN2 | — | x | x | x | x | x | x |
| CALM3 | — | — | x | x | x | x | x |
| NSUN5 | — | — | x | x | x | x | x |
| C16orf53 | — | — | x | x | x | x | x |
| HEATR3 | — | x | — | x | x | x | x |
| SMARCB1 | — | — | x | x | x | x | x |
| SARM1 | — | — | — | x | x | x | x |
| TMEM5 | — | — | — | x | x | x | x |
| SIPA1L2 | — | — | x | — | — | x | x |
| NCOA4 | x | x | x | x | x | x | — |
| RPUSD1 | x | x | x | x | x | x | — |
| ZFAND5 | x | x | x | x | x | x | — |
| SNX27 | x | x | x | x | x | x | — |
| XRCC1 | x | x | x | x | — | x | — |
| ADRB2 | x | x | x | — | — | x | — |
| PRKAR1B | x | x | x | — | — | x | — |
| TM9SF1 | x | x | — | — | x | x | — |
| FLT3LG | x | — | — | — | x | x | — |
| MT1H | x | x | — | — | — | x | — |
| PAIP2 | x | — | — | — | — | x | — |
| SNURF | — | x | x | — | x | x | — |
| CCDC6 | — | — | — | x | — | x | — |
| NR2F6 | — | x | — | — | — | x | — |
| SERPINB9 | — | — | x | — | — | x | — |
| MPP6 | x | x | x | x | x | — | — |
| SELM | x | x | x | x | x | — | — |
| CRTAP | x | x | x | x | x | — | — |
| RPS20 | x | x | x | x | x | — | — |
| C5orf32 | x | x | x | x | x | — | — |
| MAF | x | x | x | x | x | — | — |
| C20orf111 | x | x | x | — | x | — | — |
| KIAA1949 | x | — | x | x | x | — | — |
| RPL19 | x | x | x | — | x | — | — |
| AAK1 | x | — | — | x | x | — | — |
| KLF12 | x | x | x | — | — | — | — |
| LAPTM5 | x | — | x | x | — | — | — |
| C8orf33 | x | x | — | — | — | — | — |
| GCH1 | x | — | — | x | — | — | — |
| URM1 | — | — | x | x | x | — | — |
| HMGB2 | — | x | x | — | — | — | — |
| SPG7 | — | — | — | x | x | — | — |
| AP2A1 | — | x | — | — | — | — | — |
| IL1RN | — | x | — | — | — | — | — |
| MT1G | — | x | — | — | — | — | — |
| TPT1 | — | — | — | x | — | — | — |

(An “x” in the column indicates that the SG in the corresponding row is a member of the Vmod listed in the column; otherwise the “—” indicates the RGP is not a component of the Vmod)

In general, for each and every Vmod, the ultimate GVHD N Outcome Score (GNOS) is calculated from the RGP values for each sample as follows (as also described above for general voting model (Vmod) implementations): For each RGP, if its value is above or below a defined threshold (LDA separatrix; defined as the midpoint between the GVHD negative and positive population average RGP values), the sample is classified as N (negative), i.e., not leading to aGVHD in the transplantation. N votes are counted as “1”, otherwise counted as “0”, and averaged over all of the RGPs in a particular voting model to arrive at the GNOS.

Greedy Optimization of RGP Vmods:

Multiple sources of empirical, data-derived evidence have contributed to the selection of the 100 RGPs (Table 17, VmodRGP100) as outcome predictive high-performance RGP candidates for GVHD outcome prediction implementations of differing selections of 48 total SGs, through Vmods incorporating varying numbers of RGPs. These sources of evidence range from individual RGP performance, compound RGP in PRGP performance, and integrated RGP performance in Vmods designed with alternative RGP selection criteria.

Thus, an RGP optimization (instead of RGP prioritization, as used above) procedure applied in the design of the GVHD outcome prediction Vmod might result in superior Vmod performance. Accordingly, the following, simple “greedy” search/aggregation procedure was applied for Vmod optimization as described below:
(1) Vmod: SG43RGP36-RGPgreedysearch.
   The greedy search begins by selection of the best performing RGP from Table 17 (VmodRGP100 list), which defines Vmod(1). 100 minus 1 alternative Vmods are then evaluated by combining the RGP in Vmod(1) with the remaining 100 minus 1 RGPs, and the outcome predictive performance is determined for each of these 100 minus 1 Vmods. From this list of 100 minus 1 Vmods, the best performing Vmod is selected as Vmod(2). Then 100 minus 2 alternative Vmods are evaluated by combining the RGPs in Vmod(2) with the remaining 100 minus 2 RGPs, and the outcome predictive performance is determined for each of these 100 minus 2 Vmods. From this list of 100 minus 2 Vmods, the best performing Vmod is selected as Vmod(3). In general, 100 minus i alternative Vmods are evaluated by combining the RGPs in Vmod(i) with the remaining 100 minus i RGPs, and the outcome predictive performance is determined for each of these 100 minus i Vmods. From this list of 100 minus i Vmods, the best performing Vmod is selected as Vmod(i+1). The index, i, is then increased by 1, until i=101, at which the search is terminated and all the RGPs have been aggregated. Vmod Gneg vs. Gag3 T-test p-values are determined from the GNOS of each sample. All Vmods are evaluated at a GNOS threshold of 0.55, i.e., at least 55% of the constituent RGP voters in each Vmod must have cast a vote 1, i.e., an N vote (no GVHD) for a given sample. Balanced CMCVs are determined from the 1 (N) and 0 (not N) values determined by a candidate Vmod for each of the Gneg and Gag3 samples.
   The best performing Vmod from the i Vmod candidates at each iteration is selected according to the following criteria:
      1. For the division Gneg vs. Gag3, the TNR (true negative rate, specificity) must be >=0.4 for a Vmod to be considered.
      2. The best ranking Vmod is selected, according to the best average rank of the NPV (class numbers-wise balanced negative predictive value, i.e. P_{b}=0.5) rank and p-value rank for the Gneg vs. Gag3 division.
   After 36 iterations of the greedy search, a total of 36 RGPs were selected, containing 43 unique SGs, including one of the HSK6 SGs (see Table 19, VmodGreedySearch). Combining these 43 SGs with the remaining 5 HSK6 SGs, results in a total of 48 SGs for implementation as a GVHD outcome prediction test.
(2) Vmod: SG21RGP28-RGPmaxgreedysearch.
   Note that the best performing Vmod in the greedy search outlined above, in terms of Gneg vs. Gag3 combined NPV (0.96), ACC (0.90) and p-value (2.04×10⁻²³), occurs at 21 iterations of RGP selections, i.e. representing 21 unique SGs that participate in 28 different RGPs (SG21RGP28, see Table 19, VmodGreedySearch). While this Vmod could be implemented as the GVHD outcome prediction test with potentially superior performance, it is neither clear nor certain that SG21RGP28-RGPmaxgreedysearch, using less than half the SGs compared to SG43RGP36-RGPgreedysearch, would perform as consistently or robustly as SG43RGP36-RGPgreedysearch in routine GVHD outcome prediction practice.

TABLE 19
Greedy search performance for Vmods SG43GRP36 and SG21RGP28 (“VmodGreedySearch”)

| | | | Gneg vs. | Gneg vs. | Gneg vs. | | | | ACC |
|---|---|---|---|---|---|---|---|---|---|
| Greedy search | | | Gpos T- | Gag2 T- | Gag3 T- | NPV | TNR Gneg | TPR Gneg | Gneg |
| iteration, | | | test log10 | test log10 | test log10 | Gneg | vs. Gpos | vs. Gpos | vs. |
| GPs per Vmod | Select Vmod | RGP ABI Symbol | p-value | p-value | p-value | vs. Gpos | (specificity) | (sensitivity) | Gpos |
| 1 | | AEBP1-SEC14L1 | −4.69 | −4.55 | −5.90 | 0.68 | 0.63 | 0.71 | 0.67 |
| 2 | | MRPL42-FOXN2 | −5.82 | −6.25 | −8.28 | 0.77 | 0.41 | 0.88 | 0.64 |
| 3 | | PDCD6IP-LYRM5 | −9.00 | −9.19 | −12.16 | 0.76 | 0.64 | 0.79 | 0.72 |
| 4 | | MRPL42-GINS1 | −12.28 | −12.38 | −15.63 | 0.81 | 0.56 | 0.87 | 0.71 |
| 5 | | TMEM8B-C16orf53 | −14.31 | −14.66 | −17.20 | 0.79 | 0.80 | 0.79 | 0.79 |
| 6 | | TMEM8B-SIPA1L2 | −14.44 | −14.74 | −17.27 | 0.82 | 0.66 | 0.86 | 0.76 |
| 7 | | CALM3-HEATR3 | −14.43 | −15.11 | −17.77 | 0.77 | 0.80 | 0.77 | 0.78 |
| 8 | | ABHD12-CALM3 | −14.63 | −15.16 | −18.28 | 0.81 | 0.66 | 0.84 | 0.75 |
| 9 | | MPP5-SEC14L1 | −14.33 | −14.90 | −18.45 | 0.78 | 0.78 | 0.79 | 0.78 |
| 10 | | ANAPC11-GINS1 | −15.48 | −15.86 | −19.22 | 0.80 | 0.71 | 0.83 | 0.77 |
| 11 | | PREX1-SMARCB1 | −15.58 | −16.00 | −19.55 | 0.83 | 0.68 | 0.86 | 0.77 |
| 12 | | AEBP1-NCDN | −16.91 | −17.18 | −20.85 | 0.82 | 0.76 | 0.83 | 0.80 |
| 13 | | VAMP2-TMEM5 | −17.80 | −17.93 | −21.46 | 0.83 | 0.73 | 0.85 | 0.79 |
| 14 | | TMEM49-CALM3 | −18.63 | −18.56 | −22.14 | 0.83 | 0.81 | 0.83 | 0.82 |
| 15 | | MT1E-GINS1 | −19.55 | −19.48 | −22.64 | 0.85 | 0.75 | 0.87 | 0.81 |
| 16 | | TMEM8B-C5orf62 | −18.87 | −18.94 | −22.47 | 0.83 | 0.80 | 0.83 | 0.87 |
| 17 | | PDCD6IP-TATDN1 | −18.93 | −19.09 | −22.59 | 0.84 | 0.76 | 0.85 | 0.81 |
| 18 | | AEBP1-SARM1 | −19.11 | −19.15 | −22.47 | 0.84 | 0.81 | 0.84 | 0.83 |
| 19 | | TMEM8B-NSUN5 | −19.03 | −19.23 | −22.12 | 0.85 | 0.78 | 0.86 | 0.82 |
| 20 | | PLAC8-SEC14L1 | −18.65 | −19.01 | −21.97 | 0.84 | 0.83 | 0.84 | 0.84 |
| 21 | SG21RGP28- | RPL37-GINS1 | −19.45 | −19.74 | −22.69 | 0.86 | 0.83 | 0.86 | 0.85 |
| | RGPmaxgreedy- | | | | | | | | |
| | search | | | | | | | | |
| 22 | | GINS1-MT1H | −20.19 | −20.43 | −23.10 | 0.85 | 0.81 | 0.86 | 0.84 |
| 23 | | TMEM49-MRPL42 | −20.40 | −20.58 | −23.24 | 0.84 | 0.83 | 0.84 | 0.84 |
| 24 | | VAMP2-FOXN2 | −20.22 | −20.36 | −22.91 | 0.85 | 0.81 | 0.85 | 0.83 |
| 25 | | TMEM8B-TM9SF1 | −20.08 | −20.25 | −22.59 | 0.84 | 0.81 | 0.84 | 0.83 |
| 26 | | AEBP1-ZFAND5 | −20.01 | −20.16 | −22.58 | 0.84 | 0.80 | 0.85 | 0.82 |
| 27 | | TMEM49-TATDN1 | −19.31 | −19.54 | −22.18 | 0.84 | 0.81 | 0.84 | 0.83 |
| 28 | | ADRB2-MT1E | −19.17 | −19.42 | −21.93 | 0.84 | 0.80 | 0.85 | 0.82 |
| 29 | | FOXN2-SNURF | −18.93 | −19.23 | −21.76 | 0.83 | 0.81 | 0.83 | 0.82 |
| 30 | | XRCC1-SNX27 | −19.10 | −19.34 | −21.73 | 0.84 | 0.81 | 0.84 | 0.83 |
| 31 | | NR2F6-PRKAR1B | −18.89 | −19.14 | −21.55 | 0.84 | 0.78 | 0.85 | 0.82 |
| 32 | | NCOA4-PAIP2 | −18.58 | −18.86 | −21.22 | 0.84 | 0.80 | 0.84 | 0.82 |
| 33 | | MRPL42-CCDC6 | −18.15 | −18.47 | −20.90 | 0.85 | 0.78 | 0.86 | 0.82 |
| 34 | | TMEM49-FLT3LG | −18.03 | −18.35 | −20.75 | 0.84 | 0.81 | 0.84 | 0.83 |
| 35 | | VAMP2-SERPINB9 | −17.81 | −18.13 | −20.48 | 0.84 | 0.78 | 0.85 | 0.82 |
| 36 | SG43RGP36- | AEBP1-RPUSD1 | −17.95 | −18.20 | −20.48 | 0.83 | 0.80 | 0.83 | 0.82 |
| | RGPgreedy- | | | | | | | | |
| | search | | | | | | | | |

| Greedy search | NPV | TNR Gneg | TPR Gneg | ACC | NPV | TNR Gneg | TPR Gneg | ACC | Unique |
|---|---|---|---|---|---|---|---|---|---|
| iteration, | Gneg | vs. Gag2 | vs. Gag2 | Gneg | Gneg | vs. Gag3 | vs. Gag3 | Gneg | SG |
| GPs per Vmod | vs. Gag2 | (specificity) | (sensitivity) | vs. Gag2 | vs. Gag3 | (specificity) | (sensitivity) | vs. Gag3 | count |
| 1 | 0.68 | 0.63 | 0.71 | 0.67 | 0.74 | 0.63 | 0.78 | 0.70 | 2 |
| 2 | 0.79 | 0.41 | 0.89 | 0.65 | 0.94 | 0.41 | 0.97 | 0.69 | 4 |
| 3 | 0.76 | 0.64 | 0.80 | 0.72 | 0.86 | 0.64 | 0.90 | 0.77 | 6 |
| 4 | 0.81 | 0.56 | 0.87 | 0.79 | 0.93 | 0.56 | 0.96 | 0.76 | 7 |
| 5 | 0.80 | 0.80 | 0.80 | 0.80 | 0.88 | 0.80 | 0.90 | 0.85 | 9 |
| 6 | 0.84 | 0.66 | 0.87 | 0.77 | 0.94 | 0.66 | 0.96 | 0.81 | 10 |
| 7 | 0.80 | 0.80 | 0.80 | 0.80 | 0.90 | 0.80 | 0.91 | 0.85 | 12 |
| 8 | 0.82 | 0.66 | 0.85 | 0.76 | 0.94 | 0.66 | 0.96 | 0.81 | 13 |
| 9 | 0.80 | 0.78 | 0.81 | 0.79 | 0.92 | 0.78 | 0.94 | 0.86 | 14 |
| 10 | 0.82 | 0.71 | 0.85 | 0.78 | 0.95 | 0.71 | 0.96 | 0.84 | 15 |
| 11 | 0.85 | 0.68 | 0.88 | 0.78 | 0.96 | 0.68 | 0.97 | 0.83 | 17 |
| 12 | 0.84 | 0.76 | 0.85 | 0.81 | 0.94 | 0.76 | 0.95 | 0.86 | 18 |
| 13 | 0.85 | 0.73 | 0.87 | 0.80 | 0.95 | 0.73 | 0.96 | 0.84 | 20 |
| 14 | 0.85 | 0.81 | 0.85 | 0.83 | 0.94 | 0.81 | 0.95 | 0.88 | 21 |
| 15 | 0.85 | 0.75 | 0.87 | 0.81 | 0.95 | 0.75 | 0.96 | 0.85 | 22 |
| 16 | 0.84 | 0.80 | 0.85 | 0.89 | 0.94 | 0.80 | 0.95 | 0.87 | 23 |
| 17 | 0.85 | 0.76 | 0.86 | 0.81 | 0.95 | 0.76 | 0.96 | 0.86 | 24 |
| 18 | 0.85 | 0.81 | 0.85 | 0.83 | 0.94 | 0.81 | 0.95 | 0.88 | 25 |
| 19 | 0.86 | 0.78 | 0.87 | 0.83 | 0.95 | 0.78 | 0.96 | 0.87 | 26 |
| 20 | 0.86 | 0.83 | 0.86 | 0.85 | 0.96 | 0.83 | 0.96 | 0.90 | 27 |
| 21 | 0.87 | 0.83 | 0.87 | 0.85 | 0.96 | 0.83 | 0.96 | 0.90 | 28 |
| 22 | 0.86 | 0.81 | 0.87 | 0.84 | 0.95 | 0.81 | 0.96 | 0.89 | 29 |
| 23 | 0.85 | 0.83 | 0.85 | 0.84 | 0.94 | 0.83 | 0.95 | 0.89 | 29 |
| 24 | 0.86 | 0.81 | 0.86 | 0.84 | 0.95 | 0.81 | 0.96 | 0.89 | 29 |
| 25 | 0.85 | 0.81 | 0.85 | 0.83 | 0.94 | 0.81 | 0.95 | 0.88 | 30 |
| 26 | 0.85 | 0.80 | 0.86 | 0.83 | 0.95 | 0.80 | 0.96 | 0.88 | 31 |
| 27 | 0.85 | 0.81 | 0.85 | 0.83 | 0.94 | 0.81 | 0.95 | 0.88 | 31 |
| 28 | 0.85 | 0.80 | 0.86 | 0.83 | 0.95 | 0.80 | 0.96 | 0.88 | 32 |
| 29 | 0.84 | 0.81 | 0.85 | 0.83 | 0.93 | 0.81 | 0.94 | 0.87 | 33 |
| 30 | 0.85 | 0.81 | 0.85 | 0.83 | 0.94 | 0.81 | 0.95 | 0.88 | 35 |
| 31 | 0.85 | 0.78 | 0.86 | 0.82 | 0.95 | 0.78 | 0.96 | 0.87 | 37 |
| 32 | 0.85 | 0.80 | 0.85 | 0.83 | 0.94 | 0.80 | 0.95 | 0.87 | 39 |
| 33 | 0.85 | 0.78 | 0.86 | 0.82 | 0.95 | 0.78 | 0.96 | 0.87 | 40 |
| 34 | 0.85 | 0.81 | 0.85 | 0.83 | 0.94 | 0.81 | 0.95 | 0.88 | 41 |
| 35 | 0.85 | 0.78 | 0.86 | 0.82 | 0.95 | 0.78 | 0.96 | 0.87 | 42 |
| 36 | 0.84 | 0.80 | 0.85 | 0.82 | 0.92 | 0.80 | 0.94 | 0.87 | 43 |

Determination and Evaluation of Outcome Predictive Performance of RGP Vmods:

The overall GVHD outcome predictive performance of all 7 Vmods is summarized in Table 20 (VmodSpecs). For all 3 divisions (Gneg vs. Gpos, Gneg vs. Gag2, Gneg vs. Gag3), the following standard specifications (SSPCs) for outcome predictive performance are reported:
(1) Heteroscedastic, 2-tailed, T-test p-values, based on GNOS values reported for each sample in the Vmod output.
(2) For 5 different GNOS separatrices (GNOS threshold value at or above which a donor sample is ultimately classified as N, i.e. not causing GVHD in the recipient), of 0.50, 0.55, 0.65, 0.75 and 0.85, 5 additional outcome predictive SSPCs (for example, see definition of CMCVs above).
   1. NPV, negative predictive value, balanced, i.e., adjusted for equal proportions of real N outcomes (TN+FP) and real P outcomes (TP+FN), i.e. for balanced prevalence P_{b}=0.5

NPV=TN_{b}/(TN_{b}+FN_{b})
2. TNR, true negative rate, i.e., specificity (unaffected by proportions of real N outcomes and real P outcomes)

TNR=TN(TN+FP)
3. PPV, positive predictive value, balanced, i.e., adjusted for equal proportions of real N outcomes (TN+FP) and real P outcomes (TP+FN), i.e. for balanced prevalence P_{b}=0.5

PPV=TP_{b}/(TP_{b}+FP_{b})
4. TPR, true positive rate, i.e., sensitivity (unaffected by proportions of real N outcomes and real P outcomes)

TPR=TP/(TP+FN)
5. ACC, accuracy, balanced, i.e., adjusted for equal proportions of real N outcomes (TN+FP) and real P outcomes (TP+FN), i.e. for balanced prevalence P_{b}=0.5

ACC=(TN_{b}+TP_{b})/(TN_{b}+FN_{b}+TP_{b}+FP_{b})

The following observations concern Table 20 (VmodSpecs):
(1) Note that the NPV for the Gneg vs. Gag3 division at GNOS separatrices of 0.75 or 0.85 often reaches values>=0.90, however often also accompanied by TNR values<=0.25.
(2) Note that for the Gneg vs. Gag3 division, the Vmod SG43RGP36-RGPgreedysearch, at GNOS threshold 0.55, reaches the highest accuracy (0.87) of any Vmod at any division with 42 or 43 SGs, also combining an NPV value of 0.92 with a TNR (specificity) value of 0.80, and with a TPR (sensitivity) value of 0.94.
(3) Note that for all 3 divisions, and for all Vmods with 42 or 43 SGs, the Vmod SG43RGP36-RGPgreedysearch shows by far the lowest (best) T-test p-values, ranging from 1.1×10⁻¹⁸ for the Gneg vs. Gpos division, to as low as 3.3×10⁻¹⁹ for the Gneg vs. Gag3 division.
(4) Note that SG21RGP28-RGPmaxgreedysearch shows by far the best SSPCs in every category. While this Vmod could be implemented as GVHD outcome prediction test with potentially superior performance, SG21RGP28-RGPmaxgreedysearch, using less than half the SGs compared to SG43RGP36-RGPgreedysearch, may not perform as consistently or robustly as SG43RGP36-RGPgreedysearch in routine GVHD outcome prediction practice.

TABLE 20
Overall outcome predictive performance for selected 7 Vmods (“VmodSpecs”) at prevalence, P = 0.5

| | Gneg vs. | Gneg vs. | Gneg vs. | | NPV | | | ACC |
|---|---|---|---|---|---|---|---|---|
| | Gpos | Gag2 | Gag3 | | Gneg | TNR Gneg | TPR Gneg | Gneg |
| | T-test | T-test | T-test | GNOS | vs. | vs. Gpos | vs. Gpos | vs. |
| Vmod | p-value | p-value | p-value | threshold | Gpos | (specificity) | (sensitivity) | Gpos |
| SG43RGP46-RGPperformance | 1.0E−11 | 8.3E−12 | 5.4E−13 | 0.50 | 0.75 | 0.78 | 0.74 | 0.76 |
| SG43RGP46-RGPperformance | 1.0E−11 | 8.3E−12 | 5.4E−13 | 0.55 | 0.73 | 0.66 | 0.75 | 0.71 |
| SG43RGP46-RGPperformance | 1.0E−11 | 8.3E−12 | 5.4E−13 | 0.65 | 0.78 | 0.54 | 0.84 | 0.69 |
| SG43RGP46-RGPperformance | 1.0E−11 | 8.3E−12 | 5.4E−13 | 0.75 | 0.77 | 0.42 | 0.88 | 0.65 |
| SG43RGP46-RGPperformance | 1.0E−11 | 8.3E−12 | 5.4E−13 | 0.85 | 0.79 | 0.25 | 0.93 | 0.59 |
| SG42RGP21-RGPminimalist | 1.8E−11 | 1.1E−11 | 1.2E−12 | 0.50 | 0.72 | 0.71 | 0.73 | 0.72 |
| SG42RGP21-RGPminimalist | 1.8E−11 | 1.1E−11 | 1.2E−12 | 0.55 | 0.77 | 0.66 | 0.80 | 0.73 |
| SG42RGP21-RGPminimalist | 1.8E−11 | 1.1E−11 | 1.2E−12 | 0.65 | 0.78 | 0.54 | 0.85 | 0.70 |
| SG42RGP21-RGPminimalist | 1.8E−11 | 1.1E−11 | 1.2E−12 | 0.75 | 0.79 | 0.42 | 0.88 | 0.65 |
| SG42RGP21-RGPminimalist | 1.8E−11 | 1.1E−11 | 1.2E−12 | 0.85 | 0.88 | 0.25 | 0.97 | 0.61 |
| SG43RGP37-RGPconnectivity | 5.5E−11 | 2.8E−11 | 1.6E−12 | 0.50 | 0.73 | 0.75 | 0.72 | 0.73 |
| SG43RGP37-RGPconnectivity | 5.5E−11 | 2.8E−11 | 1.6E−12 | 0.55 | 0.75 | 0.68 | 0.78 | 0.73 |
| SG43RGP37-RGPconnectivity | 5.5E−11 | 2.8E−11 | 1.6E−12 | 0.65 | 0.78 | 0.53 | 0.85 | 0.69 |
| SG43RGP37-RGPconnectivity | 5.5E−11 | 2.8E−11 | 1.6E−12 | 0.75 | 0.80 | 0.46 | 0.88 | 0.67 |
| SG43RGP37-RGPconnectivity | 5.5E−11 | 2.8E−11 | 1.6E−12 | 0.85 | 0.77 | 0.25 | 0.93 | 0.59 |
| SG43RGP51-PRGPminranksort | 9.3E−12 | 5.7E−12 | 1.4E−13 | 0.50 | 0.76 | 0.75 | 0.76 | 0.75 |
| SG43RGP51-PRGPminranksort | 9.3E−12 | 5.7E−12 | 1.4E−13 | 0.55 | 0.77 | 0.63 | 0.81 | 0.72 |
| SG43RGP51-PRGPminranksort | 9.3E−12 | 5.7E−12 | 1.4E−13 | 0.65 | 0.78 | 0.51 | 0.86 | 0.68 |
| SG43RGP51-PRGPminranksort | 9.3E−12 | 5.7E−12 | 1.4E−13 | 0.75 | 0.85 | 0.41 | 0.93 | 0.67 |
| SG43RGP51-PRGPminranksort | 9.3E−12 | 5.7E−12 | 1.4E−13 | 0.85 | 0.80 | 0.10 | 0.98 | 0.54 |
| SG43RGP55-PRGPmedranksort | 2.8E−11 | 2.1E−11 | 6.5E−13 | 0.50 | 0.74 | 0.76 | 0.74 | 0.75 |
| SG43RGP55-PRGPmedranksort | 2.8E−11 | 2.1E−11 | 6.5E−13 | 0.55 | 0.74 | 0.64 | 0.78 | 0.71 |
| SG43RGP55-PRGPmedranksort | 2.8E−11 | 2.1E−11 | 6.5E−13 | 0.65 | 0.77 | 0.51 | 0.85 | 0.68 |
| SG43RGP55-PRGPmedranksort | 2.8E−11 | 2.1E−11 | 6.5E−13 | 0.75 | 0.79 | 0.37 | 0.90 | 0.64 |
| SG43RGP55-PRGPmedranksort | 2.8E−11 | 2.1E−11 | 6.5E−13 | 0.85 | 0.79 | 0.15 | 0.96 | 0.56 |
| SG43RGP36-RGPgreedysearch | 1.1E−18 | 6.3E−19 | 3.3E−21 | 0.50 | 0.79 | 0.86 | 0.77 | 0.82 |
| SG43RGP36-RGPgreedysearch | 1.1E−18 | 6.3E−19 | 3.3E−21 | 0.55 | 0.83 | 0.80 | 0.83 | 0.82 |
| SG43RGP36-RGPgreedysearch | 1.1E−18 | 6.3E−19 | 3.3E−21 | 0.65 | 0.82 | 0.53 | 0.88 | 0.70 |
| SG43RGP36-RGPgreedysearch | 1.1E−18 | 6.3E−19 | 3.3E−21 | 0.75 | 0.84 | 0.25 | 0.95 | 0.60 |
| SG43RGP36-RGPgreedysearch | 1.1E−18 | 6.3E−19 | 3.3E−21 | 0.85 | 0.86 | 0.05 | 0.99 | 0.52 |
| SG21RGP28-RGPmaxgreedysearch | 3.6E−20 | 1.8E−20 | 2.0E−23 | 0.50 | 0.81 | 0.83 | 0.80 | 0.82 |
| SG21RGP28-RGPmaxgreedysearch | 3.6E−20 | 1.8E−20 | 2.0E−23 | 0.55 | 0.86 | 0.83 | 0.86 | 0.85 |
| SG21RGP28-RGPmaxgreedysearch | 3.6E−20 | 1.8E−20 | 2.0E−23 | 0.65 | 0.79 | 0.47 | 0.88 | 0.68 |
| SG21RGP28-RGPmaxgreedysearch | 3.6E−20 | 1.8E−20 | 2.0E−23 | 0.75 | 0.86 | 0.25 | 0.96 | 0.61 |
| SG21RGP28-RGPmaxgreedysearch | 3.6E−20 | 1.8E−20 | 2.0E−23 | 0.85 | 0.61 | 0.05 | 0.97 | 0.51 |

| | NPV | | | ACC | NPV | | | ACC |
|---|---|---|---|---|---|---|---|---|
| | Gneg | TNR Gneg | TPR Gneg | Gneg | Gneg | TNR Gneg | TPR Gneg | Gneg |
| | vs. | vs. Gag2 | vs. Gag2 | vs. | vs. | vs. Gag3 | vs. Gag3 | vs. |
| Vmod | Gag2 | (specificity) | (sensitivity) | Gag2 | Gag3 | (specificity) | (sensitivity) | Gag3 |
| SG43RGP46-RGPperformance | 0.75 | 0.78 | 0.75 | 0.76 | 0.80 | 0.78 | 0.81 | 0.79 |
| SG43RGP46-RGPperformance | 0.72 | 0.66 | 0.75 | 0.70 | 0.77 | 0.66 | 0.81 | 0.73 |
| SG43RGP46-RGPperformance | 0.78 | 0.54 | 0.85 | 0.69 | 0.86 | 0.54 | 0.91 | 0.73 |
| SG43RGP46-RGPperformance | 0.77 | 0.42 | 0.87 | 0.65 | 0.84 | 0.42 | 0.92 | 0.67 |
| SG43RGP46-RGPperformance | 0.80 | 0.25 | 0.94 | 0.60 | 0.91 | 0.25 | 0.97 | 0.61 |
| SG42RGP21-RGPminimalist | 0.72 | 0.71 | 0.73 | 0.72 | 0.76 | 0.71 | 0.78 | 0.75 |
| SG42RGP21-RGPminimalist | 0.77 | 0.66 | 0.80 | 0.73 | 0.85 | 0.66 | 0.88 | 0.77 |
| SG42RGP21-RGPminimalist | 0.79 | 0.54 | 0.85 | 0.70 | 0.87 | 0.54 | 0.92 | 0.73 |
| SG42RGP21-RGPminimalist | 0.80 | 0.42 | 0.89 | 0.66 | 0.87 | 0.42 | 0.94 | 0.68 |
| SG42RGP21-RGPminimalist | 0.90 | 0.25 | 0.97 | 0.61 | 1.00 | 0.25 | 1.00 | 0.63 |
| SG43RGP37-RGPconnectivity | 0.73 | 0.75 | 0.73 | 0.74 | 0.77 | 0.75 | 0.78 | 0.76 |
| SG43RGP37-RGPconnectivity | 0.76 | 0.68 | 0.79 | 0.73 | 0.84 | 0.68 | 0.87 | 0.77 |
| SG43RGP37-RGPconnectivity | 0.78 | 0.53 | 0.85 | 0.69 | 0.89 | 0.53 | 0.94 | 0.73 |
| SG43RGP37-RGPconnectivity | 0.81 | 0.46 | 0.89 | 0.67 | 0.90 | 0.46 | 0.95 | 0.70 |
| SG43RGP37-RGPconnectivity | 0.78 | 0.25 | 0.93 | 0.59 | 0.91 | 0.25 | 0.97 | 0.61 |
| SG43RGP51-PRGPminranksort | 0.77 | 0.75 | 0.77 | 0.76 | 0.82 | 0.75 | 0.83 | 0.79 |
| SG43RGP51-PRGPminranksort | 0.78 | 0.63 | 0.83 | 0.73 | 0.86 | 0.63 | 0.90 | 0.76 |
| SG43RGP51-PRGPminranksort | 0.79 | 0.51 | 0.86 | 0.69 | 0.89 | 0.51 | 0.94 | 0.72 |
| SG43RGP51-PRGPminranksort | 0.85 | 0.41 | 0.93 | 0.67 | 0.91 | 0.41 | 0.96 | 0.68 |
| SG43RGP51-PRGPminranksort | 0.79 | 0.10 | 0.97 | 0.54 | 1.00 | 0.10 | 1.00 | 0.55 |
| SG43RGP55-PRGPmedranksort | 0.75 | 0.76 | 0.75 | 0.75 | 0.80 | 0.76 | 0.81 | 0.78 |
| SG43RGP55-PRGPmedranksort | 0.75 | 0.64 | 0.79 | 0.72 | 0.82 | 0.64 | 0.86 | 0.75 |
| SG43RGP55-PRGPmedranksort | 0.78 | 0.51 | 0.85 | 0.68 | 0.87 | 0.51 | 0.92 | 0.72 |
| SG43RGP55-PRGPmedranksort | 0.79 | 0.37 | 0.90 | 0.64 | 0.88 | 0.37 | 0.95 | 0.66 |
| SG43RGP55-PRGPmedranksort | 0.81 | 0.15 | 0.96 | 0.56 | 1.00 | 0.15 | 1.00 | 0.58 |
| SG43RGP36-RGPgreedysearch | 0.79 | 0.86 | 0.77 | 0.82 | 0.84 | 0.86 | 0.83 | 0.85 |
| SG43RGP36-RGPgreedysearch | 0.84 | 0.80 | 0.85 | 0.82 | 0.92 | 0.80 | 0.94 | 0.87 |
| SG43RGP36-RGPgreedysearch | 0.83 | 0.53 | 0.89 | 0.71 | 0.93 | 0.53 | 0.96 | 0.74 |
| SG43RGP36-RGPgreedysearch | 0.85 | 0.25 | 0.95 | 0.60 | 0.91 | 0.25 | 0.97 | 0.61 |
| SG43RGP36-RGPgreedysearch | 0.85 | 0.05 | 0.99 | 0.52 | 1.00 | 0.05 | 1.00 | 0.53 |
| SG21RGP28-RGPmaxgreedysearch | 0.82 | 0.83 | 0.82 | 0.82 | 0.90 | 0.83 | 0.91 | 0.87 |
| SG21RGP28-RGPmaxgreedysearch | 0.87 | 0.83 | 0.87 | 0.85 | 0.96 | 0.83 | 0.96 | 0.90 |
| SG21RGP28-RGPmaxgreedysearch | 0.79 | 0.47 | 0.87 | 0.67 | 0.92 | 0.47 | 0.96 | 0.72 |
| SG21RGP28-RGPmaxgreedysearch | 0.85 | 0.25 | 0.95 | 0.60 | 0.91 | 0.25 | 0.97 | 0.61 |
| SG21RGP28-RGPmaxgreedysearch | 0.58 | 0.05 | 0.96 | 0.51 | 0.80 | 0.05 | 0.99 | 0.52 |

The observed and balanced (adjusted for equal proportions of real N outcomes and real P outcomes), absolute or relative (for adjusted values) “confusion matrix” counts of correctly and incorrectly classified samples (TN, FP, TP, FN; bTN, bFP, bTP, bFN), for the 5 different GNOS separatrices (0.50, 0.55, 0.65, 0.75 and 0.85) from which all of the 5 outcome predictive accuracies/proportions (NPV, TNR, PPV, TPR, ACC) were calculated, are reported in Table 21 (VmodCounts).

TABLE 21
Correct and incorrect sample classification counts and balanced
relative counts for selected 7 Vmods (“VmodCounts”)

| | GNOS | TN | FP | TP | FN | TP | FN | TP | FN | bTN |
|---|---|---|---|---|---|---|---|---|---|---|
| Vmod | threshold | Gneg | Gneg | Gpos | Gpos | Gag2 | Gag2 | Gag3 | Gag3 | Gneg |
| SG43RGP46-RGPperformance | 0.50 | 46 | 13 | 90 | 31 | 82 | 28 | 62 | 15 | 0.39 |
| SG43RGP46-RGPperformance | 0.55 | 39 | 20 | 91 | 30 | 82 | 28 | 62 | 15 | 0.33 |
| SG43RGP46-RGPperformance | 0.65 | 32 | 27 | 102 | 19 | 93 | 17 | 70 | 7 | 0.27 |
| SG43RGP46-RGPperformance | 0.75 | 25 | 34 | 106 | 15 | 96 | 14 | 71 | 6 | 0.21 |
| SG43RGP46-RGPperformance | 0.85 | 15 | 44 | 113 | 8 | 103 | 7 | 75 | 2 | 0.13 |
| SG42RGP21-RGPminimalist | 0.50 | 42 | 17 | 88 | 33 | 80 | 30 | 60 | 17 | 0.36 |
| SG42RGP21-RGPminimalist | 0.55 | 39 | 20 | 97 | 24 | 88 | 22 | 68 | 9 | 0.33 |
| SG42RGP21-RGPminimalist | 0.65 | 32 | 27 | 103 | 18 | 94 | 16 | 71 | 6 | 0.27 |
| SG42RGP21-RGPminimalist | 0.75 | 25 | 34 | 107 | 14 | 98 | 12 | 72 | 5 | 0.21 |
| SG42RGP21-RGPminimalist | 0.85 | 15 | 44 | 117 | 4 | 107 | 3 | 77 | 0 | 0.13 |
| SG43RGP37-RGPconnectivity | 0.50 | 44 | 15 | 87 | 34 | 80 | 30 | 60 | 17 | 0.37 |
| SG43RGP37-RGPconnectivity | 0.55 | 40 | 19 | 94 | 27 | 87 | 23 | 67 | 10 | 0.34 |
| SG43RGP37-RGPconnectivity | 0.65 | 31 | 28 | 103 | 18 | 94 | 16 | 72 | 5 | 0.26 |
| SG43RGP37-RGPconnectivity | 0.75 | 27 | 32 | 107 | 14 | 98 | 12 | 73 | 4 | 0.23 |
| SG43RGP37-RGPconnectivity | 0.85 | 15 | 44 | 112 | 9 | 102 | 8 | 75 | 2 | 0.13 |
| SG43RGP51-PRGPminranksort | 0.50 | 44 | 15 | 92 | 29 | 85 | 25 | 64 | 13 | 0.37 |
| SG43RGP51-PRGPminranksort | 0.55 | 37 | 22 | 98 | 23 | 91 | 19 | 69 | 8 | 0.31 |
| SG43RGP51-PRGPminranksort | 0.65 | 30 | 29 | 104 | 17 | 95 | 15 | 72 | 5 | 0.25 |
| SG43RGP51-PRGPminranksort | 0.75 | 24 | 35 | 112 | 9 | 102 | 8 | 74 | 3 | 0.20 |
| SG43RGP51-PRGPminranksort | 0.85 | 6 | 53 | 118 | 3 | 107 | 3 | 77 | 0 | 0.05 |
| SG43RGP55-PRGPmedranksort | 0.50 | 45 | 14 | 89 | 32 | 82 | 28 | 62 | 15 | 0.38 |
| SG43RGP55-PRGPmedranksort | 0.55 | 38 | 21 | 94 | 27 | 87 | 23 | 66 | 11 | 0.32 |
| SG43RGP55-PRGPmedranksort | 0.65 | 30 | 29 | 103 | 18 | 94 | 16 | 71 | 6 | 0.25 |
| SG43RGP55-PRGPmedranksort | 0.75 | 22 | 37 | 109 | 12 | 99 | 11 | 73 | 4 | 0.19 |
| SG43RGP55-PRGPmedranksort | 0.85 | 9 | 50 | 116 | 5 | 106 | 4 | 77 | 0 | 0.08 |
| SG43RGP36-RGPgreedysearch | 0.50 | 51 | 8 | 93 | 28 | 85 | 25 | 64 | 13 | 0.43 |
| SG43RGP36-RGPgreedysearch | 0.55 | 47 | 12 | 101 | 20 | 93 | 17 | 72 | 5 | 0.40 |
| SG43RGP36-RGPgreedysearch | 0.65 | 31 | 28 | 107 | 14 | 98 | 12 | 74 | 3 | 0.26 |
| SG43RGP36-RGPgreedysearch | 0.75 | 15 | 44 | 115 | 6 | 105 | 5 | 75 | 2 | 0.13 |
| SG43RGP36-RGPgreedysearch | 0.85 | 3 | 56 | 120 | 1 | 109 | 1 | 77 | 0 | 0.03 |
| SG21RGP28-RGPmaxgreedysearch | 0.50 | 49 | 10 | 97 | 24 | 90 | 20 | 70 | 7 | 0.42 |
| SG21RGP28-RGPmaxgreedysearch | 0.55 | 49 | 10 | 104 | 17 | 96 | 14 | 74 | 3 | 0.42 |
| SG21RGP28-RGPmaxgreedysearch | 0.65 | 28 | 31 | 106 | 15 | 96 | 14 | 74 | 3 | 0.24 |
| SG21RGP28-RGPmaxgreedysearch | 0.75 | 15 | 44 | 116 | 5 | 105 | 5 | 75 | 2 | 0.13 |
| SG21RGP28-RGPmaxgreedysearch | 0.85 | 3 | 56 | 117 | 4 | 106 | 4 | 76 | 1 | 0.03 |

| | bFP | bTP | bFN | bTP | bFN | bTP | bFN |
|---|---|---|---|---|---|---|---|
| Vmod | Gneg | Gpos | Gpos | Gag2 | Gag2 | Gag3 | Gag3 |
| SG43RGP46-RGPperformance | 0.11 | 0.37 | 0.13 | 0.37 | 0.13 | 0.40 | 0.10 |
| SG43RGP46-RGPperformance | 0.17 | 0.38 | 0.12 | 0.37 | 0.13 | 0.40 | 0.10 |
| SG43RGP46-RGPperformance | 0.23 | 0.42 | 0.08 | 0.42 | 0.08 | 0.45 | 0.05 |
| SG43RGP46-RGPperformance | 0.29 | 0.44 | 0.06 | 0.44 | 0.06 | 0.46 | 0.04 |
| SG43RGP46-RGPperformance | 0.37 | 0.47 | 0.03 | 0.47 | 0.03 | 0.49 | 0.01 |
| SG42RGP21-RGPminimalist | 0.14 | 0.36 | 0.14 | 0.36 | 0.14 | 0.39 | 0.11 |
| SG42RGP21-RGPminimalist | 0.17 | 0.40 | 0.10 | 0.40 | 0.10 | 0.44 | 0.06 |
| SG42RGP21-RGPminimalist | 0.23 | 0.43 | 0.07 | 0.43 | 0.07 | 0.46 | 0.04 |
| SG42RGP21-RGPminimalist | 0.29 | 0.44 | 0.06 | 0.45 | 0.05 | 0.47 | 0.03 |
| SG42RGP21-RGPminimalist | 0.37 | 0.48 | 0.02 | 0.49 | 0.01 | 0.50 | 0.00 |
| SG43RGP37-RGPconnectivity | 0.13 | 0.36 | 0.14 | 0.36 | 0.14 | 0.39 | 0.11 |
| SG43RGP37-RGPconnectivity | 0.16 | 0.39 | 0.11 | 0.40 | 0.10 | 0.44 | 0.06 |
| SG43RGP37-RGPconnectivity | 0.24 | 0.43 | 0.07 | 0.43 | 0.07 | 0.47 | 0.03 |
| SG43RGP37-RGPconnectivity | 0.27 | 0.44 | 0.06 | 0.45 | 0.05 | 0.47 | 0.03 |
| SG43RGP37-RGPconnectivity | 0.37 | 0.46 | 0.04 | 0.46 | 0.04 | 0.49 | 0.01 |
| SG43RGP51-PRGPminranksort | 0.13 | 0.38 | 0.12 | 0.39 | 0.11 | 0.42 | 0.08 |
| SG43RGP51-PRGPminranksort | 0.19 | 0.40 | 0.10 | 0.41 | 0.09 | 0.45 | 0.05 |
| SG43RGP51-PRGPminranksort | 0.25 | 0.43 | 0.07 | 0.43 | 0.07 | 0.47 | 0.03 |
| SG43RGP51-PRGPminranksort | 0.30 | 0.46 | 0.04 | 0.46 | 0.04 | 0.48 | 0.02 |
| SG43RGP51-PRGPminranksort | 0.45 | 0.49 | 0.01 | 0.49 | 0.01 | 0.50 | 0.00 |
| SG43RGP55-PRGPmedranksort | 0.12 | 0.37 | 0.13 | 0.37 | 0.13 | 0.40 | 0.10 |
| SG43RGP55-PRGPmedranksort | 0.18 | 0.39 | 0.11 | 0.40 | 0.10 | 0.43 | 0.07 |
| SG43RGP55-PRGPmedranksort | 0.25 | 0.43 | 0.07 | 0.43 | 0.07 | 0.46 | 0.04 |
| SG43RGP55-PRGPmedranksort | 0.31 | 0.45 | 0.05 | 0.45 | 0.05 | 0.47 | 0.03 |
| SG43RGP55-PRGPmedranksort | 0.42 | 0.48 | 0.02 | 0.48 | 0.02 | 0.50 | 0.00 |
| SG43RGP36-RGPgreedysearch | 0.07 | 0.38 | 0.12 | 0.39 | 0.11 | 0.42 | 0.08 |
| SG43RGP36-RGPgreedysearch | 0.10 | 0.42 | 0.08 | 0.42 | 0.08 | 0.47 | 0.03 |
| SG43RGP36-RGPgreedysearch | 0.24 | 0.44 | 0.06 | 0.45 | 0.05 | 0.48 | 0.02 |
| SG43RGP36-RGPgreedysearch | 0.37 | 0.48 | 0.02 | 0.48 | 0.02 | 0.49 | 0.01 |
| SG43RGP36-RGPgreedysearch | 0.47 | 0.50 | 0.00 | 0.50 | 0.00 | 0.50 | 0.00 |
| SG21RGP28-RGPmaxgreedysearch | 0.08 | 0.40 | 0.10 | 0.41 | 0.09 | 0.45 | 0.05 |
| SG21RGP28-RGPmaxgreedysearch | 0.08 | 0.43 | 0.07 | 0.44 | 0.06 | 0.48 | 0.02 |
| SG21RGP28-RGPmaxgreedysearch | 0.26 | 0.44 | 0.06 | 0.44 | 0.06 | 0.48 | 0.02 |
| SG21RGP28-RGPmaxgreedysearch | 0.37 | 0.48 | 0.02 | 0.48 | 0.02 | 0.49 | 0.01 |
| SG21RGP28-RGPmaxgreedysearch | 0.47 | 0.48 | 0.02 | 0.48 | 0.02 | 0.49 | 0.01 |

Projected Medical Gains when Using GVHD Outcome Prediction:

Projected medical practice and patients' morbidity and mortality gains in GVHD outcome reduction, concomitant with projected GVHD N donor capture (GVHD N donor is defined as member of set of real, observed donors in transplantations not involving GVHD, i.e. sum of true negatives and false positives), for realistic prevalence estimates of acute grade II, III or IV GVHD (35% to 55%) and acute grade III or IV GVHD (15% to 35%) for different GNOS separatrices are summarized in Tables 22 and 23, VmodMedGainGag3 and VmodMedGainGag2, respectively. The GVHD reduction projection is based on the assumption that only donors would be used in transplants that are predicted to not cause GVHD in the recipient. In other words, assuming such stringent “N” donor selection practice, the only remaining cases of grades II, III, or IV acute GVHD should be due to any remaining false negative predictions.

The projections covering various prevalence alternatives in Tables 22 and 23 (VmodMedGainGag3 and VmodMedGainGag2) are directly and completely derived from the values listed in Table 21 (VmodCounts), i.e. sample classification counts and balanced CMCVs (confusion matrix classification values), for the selected 6 Vmods, considering varying GNOS separatrices. In addition, these Tables also report the SSPCs of NPV, TNR (specificity) and TPR (sensitivity), at the respective alternative prevalences, Pₐ, and GNOS separatrices.

To determine select SSCVs for alternative (noted by subscript “a”) prevalences, Pₐ, the 4 CMCVs need to first be adjusted as described below:

TNₐ=(1−Pₐ)/(1−P₀)*TN₀ (1)

FPₐ=(1−Pₐ)/(1−P₀)*FP₀ (2)

TPₐ=Pₐ/P₀*TP₀ (3)

FNₐ=Pₐ/P₀*FN₀ (4)

Note that for converting the balanced CMCVs listed in Table 21 “VmodCounts,” P₀=P_{b}=0.50.

Given the CMCVs adjusted for Pₐ above, the following 5 SSPCs are determined as follows:
(1) NPV, negative predictive value, adjusted for alternative prevalence, Pₐ

NPV=TNₐ/(TNₐ+FNₐ)
(2) TNR, true negative rate, specificity (unaffected by prevalence)

TNR=TN(TN+FP)
(3) PPV, positive predictive value, adjusted for alternative prevalence, Pₐ

PPV=TPₐ/(TPₐ+FPₐ)
(4) TPR, true positive rate, i.e., sensitivity (unaffected by prevalence)

TPR=TP/(TP+FN)
(5) ACC, accuracy, adjusted for alternative prevalence, Pₐ

ACC=(TNₐ+TPₐ)/(TNₐ+FNₐ+TPₐ+FPₐ)

The GVHD reduction value reported in the Tables is calculated from the respective negative predictive values (NPV) and alternative prevalences (Pₐ) according to the following equation: GVHD reduction=1−(1−NPV)/Pₐ.

When the NPV is 1, i.e. when 100% of negative classifications are correct, GVHD reduction becomes 1, i.e. 100%. When the NPV is between [1−Pₐ] and 1, the GVHD reduction ranges from 0 to 1, i.e. 0% to 100%. When the NPV is between 0 and [1−Pₐ], the GVHD reduction ranges from [1−1/Pₐ] (the lower limit of GVHD reduction, which is negative when Pₐ<1), to 0. Note that the when the NPV<[1−Pₐ], the corresponding negative GVHD reduction really means there would be an increase of GVHD. Therefore, for the GVHD outcome prediction test to be effective in GVHD reduction, it is always a requirement that NPV>[1−Pₐ].

The GVHD N donor capture value reported in the Tables is the same as the TNR value, but reported as a percentage. This value emphasizes the percentage of “real” available negative donors that would be captured by the GVHD outcome prediction test.

The following observations should be noted for Table 22 (VmodMedGainGag3), at the lowest Gag3 prevalence of 15%:
(1) The projected GVHD reduction at GNOS separatrices of 0.75 or 0.85 often reaches values>=85%, however often also accompanied by GVHD N donor capture of <=25%.
(2) Note that for Vmods with 42 or 43 SGs, the Vmod SG43RGP36-RGPgreedysearch, at GNOS threshold 0.55, reaches the highest combined 91% GVHD reduction with 80% GVHD N donor capture.
(3) For the Vmod SG42RGP21-RGPminimalist, at GNOS threshold 0.85, projects 100% GVHD reduction, however with 25% GVHD N donor capture. Furthermore, the unusually high value of 100% would likely drop to somewhere in the 90% range, as more samples are tested in the future with more behavioral diversity represented by a more complex population than has been surveyed so far.
(4) SG21RGP28-RGPmaxgreedysearch shows the highest combined GVHD reduction (95%) with GVHD N donor capture (83%) compared to all other Vmods. While this Vmod could be implemented with potentially superior performance, SG21RGP28-RGPmaxgreedysearch, using less than half the SGs compared to SG43RGP36-RGPgreedysearch, may not perform as consistently or robustly as SG43RGP36-RGPgreedysearch in routine practice.

The following observations should be note for Table 23 (VmodMedGainGag2), at the lowest Gag2 prevalence of 35%:
(1) For Vmods with 42 or 43 SGs, the Vmod SG43RGP36-RGPgreedysearch, at GNOS threshold 0.55, reaches the highest combined 73% GVHD reduction with 80% GVHD N donor capture.
(2) The Vmod SG42RGP21-RGPminimalist, at GNOS threshold 0.85, projects 84% GVHD reduction, however with 25% GVHD N donor capture.
(3) SG21RGP28-RGPmaxgreedysearch shows the highest combined GVHD reduction (78%) with GVHD N donor capture (83%) compared to all other Vmods.

TABLE 22
Projected gains in GVHD outcome reduction and GVHD N donor capture for acute grades
III or IV GVHD (“VmodMedGainGag3”), assuming prevalences ranging from 15% to 35%

| | | Alternative prevalences for Gag3 | | | | | | |
|---|---|---|---|---|---|---|---|---|
| | | | 35% | 35% | | 35% | | 25% |
| | | | TNR Gneg | TPR Gneg | 35% | GVHD N | | TNR Gneg |
| | GNOS | 35% | vs. Gag3 | vs. Gag3 | GVHD | donor | 25% | vs. Gag3 |
| | thresh- | NPV Gneg | (spec- | (sensi- | reduction | capture | NPV Gneg | (spec- |
| Vmod | old | vs. Gag3 | ificity) | tivity) | for Gag3 | of Gneg | vs. Gag3 | ificity) |
| SG43RGP46-RGPperformance | 0.50 | 0.88 | 0.78 | 0.81 | 66% | 78% | 0.92 | 0.78 |
| SG43RGP46-RGPperformance | 0.55 | 0.86 | 0.66 | 0.81 | 61% | 66% | 0.91 | 0.66 |
| SG43RGP46-RGPperformance | 0.65 | 0.92 | 0.54 | 0.91 | 76% | 54% | 0.95 | 0.54 |
| SG43RGP46-RGPperformance | 0.75 | 0.91 | 0.42 | 0.92 | 74% | 42% | 0.94 | 0.42 |
| SG43RGP46-RGPperformance | 0.85 | 0.95 | 0.25 | 0.97 | 85% | 25% | 0.97 | 0.25 |
| SG42RGP21-RGPminimalist | 0.50 | 0.86 | 0.71 | 0.78 | 59% | 71% | 0.91 | 0.71 |
| SG42RGP21-RGPminimalist | 0.55 | 0.91 | 0.66 | 0.88 | 75% | 66% | 0.94 | 0.66 |
| SG42RGP21-RGPminimalist | 0.65 | 0.93 | 0.54 | 0.92 | 79% | 54% | 0.95 | 0.54 |
| SG42RGP21-RGPminimalist | 0.75 | 0.92 | 0.42 | 0.94 | 78% | 42% | 0.95 | 0.42 |
| SG42RGP21-RGPminimalist | 0.85 | 1.00 | 0.25 | 1.00 | 100% | 25% | 1.00 | 0.25 |
| SG43RGP37-RGPconnectivity | 0.50 | 0.86 | 0.75 | 0.78 | 61% | 75% | 0.91 | 0.75 |
| SG43RGP37-RGPconnectivity | 0.55 | 0.91 | 0.68 | 0.87 | 73% | 68% | 0.94 | 0.68 |
| SG43RGP37-RGPconnectivity | 0.65 | 0.94 | 0.53 | 0.94 | 82% | 53% | 0.96 | 0.53 |
| SG43RGP37-RGPconnectivity | 0.75 | 0.94 | 0.46 | 0.95 | 84% | 46% | 0.96 | 0.46 |
| SG43RGP37-RGPconnectivity | 0.85 | 0.95 | 0.25 | 0.97 | 85% | 25% | 0.97 | 0.25 |
| SG43RGP51-PRGPminranksort | 0.50 | 0.89 | 0.75 | 0.83 | 69% | 75% | 0.93 | 0.75 |
| SG43RGP51-PRGPminranksort | 0.55 | 0.92 | 0.63 | 0.90 | 77% | 63% | 0.95 | 0.63 |
| SG43RGP51-PRGPminranksort | 0.65 | 0.94 | 0.51 | 0.94 | 82% | 51% | 0.96 | 0.51 |
| SG43RGP51-PRGPminranksort | 0.75 | 0.95 | 0.41 | 0.96 | 86% | 41% | 0.97 | 0.41 |
| SG43RGP51-PRGPminranksort | 0.85 | 1.00 | 0.10 | 1.00 | 100% | 10% | 1.00 | 0.10 |
| SG43RGP55-PRGPmedranksort | 0.50 | 0.88 | 0.76 | 0.81 | 65% | 76% | 0.92 | 0.76 |
| SG43RGP55-PRGPmedranksort | 0.55 | 0.89 | 0.64 | 0.86 | 70% | 64% | 0.93 | 0.64 |
| SG43RGP55-PRGPmedranksort | 0.65 | 0.92 | 0.51 | 0.92 | 78% | 51% | 0.95 | 0.51 |
| SG43RGP55-PRGPmedranksort | 0.75 | 0.93 | 0.37 | 0.95 | 80% | 37% | 0.96 | 0.37 |
| SG43RGP55-PRGPmedranksort | 0.85 | 1.00 | 0.15 | 1.00 | 100% | 15% | 1.00 | 0.15 |
| SG43RGP36-RGPgreedysearch | 0.50 | 0.90 | 0.86 | 0.83 | 73% | 86% | 0.94 | 0.86 |
| SG43RGP36-RGPgreedysearch | 0.55 | 0.96 | 0.80 | 0.94 | 88% | 80% | 0.97 | 0.80 |
| SG43RGP36-RGPgreedysearch | 0.65 | 0.96 | 0.53 | 0.96 | 89% | 53% | 0.98 | 0.53 |
| SG43RGP36-RGPgreedysearch | 0.75 | 0.95 | 0.25 | 0.97 | 85% | 25% | 0.97 | 0.25 |
| SG43RGP36-RGPgreedysearch | 0.85 | 1.00 | 0.05 | 1.00 | 100% | 5% | 1.00 | 0.05 |
| SG21RGP28-RGPmaxgreedysearch | 0.50 | 0.94 | 0.83 | 0.91 | 84% | 83% | 0.96 | 0.83 |
| SG21RGP28-RGPmaxgreedysearch | 0.55 | 0.98 | 0.83 | 0.96 | 93% | 83% | 0.98 | 0.83 |
| SG21RGP28-RGPmaxgreedysearch | 0.65 | 0.96 | 0.47 | 0.96 | 88% | 47% | 0.97 | 0.47 |
| SG21RGP28-RGPmaxgreedysearch | 0.75 | 0.95 | 0.25 | 0.97 | 85% | 25% | 0.97 | 0.25 |
| SG21RGP28-RGPmaxgreedysearch | 0.85 | 0.88 | 0.05 | 0.99 | 65% | 5% | 0.92 | 0.05 |

| | Alternative prevalences for Gag3 | | | | | | | |
|---|---|---|---|---|---|---|---|---|
| | 25% | | 25% | | 15% | 15% | | 15% |
| | TPR Gneg | 25% | GVHD N | | TNR Gneg | TPR Gneg | 15% | GVHD N |
| | vs. Gag3 | GVHD | donor | 15% | vs. Gag3 | vs. Gag3 | GVHD | donor |
| | (sensi- | reduction | capture of | NPV Gneg | (spec- | (sensi- | reduction | capture of |
| Vmod | tivity) | for Gag3 | Gneg | vs. Gag3 | ificity) | tivity) | for Gag3 | Gneg |
| SG43RGP46-RGPperformance | 0.81 | 69% | 78% | 0.96 | 0.78 | 0.81 | 72% | 78% |
| SG43RGP46-RGPperformance | 0.81 | 64% | 66% | 0.95 | 0.66 | 0.81 | 67% | 66% |
| SG43RGP46-RGPperformance | 0.91 | 79% | 54% | 0.97 | 0.54 | 0.91 | 81% | 54% |
| SG43RGP46-RGPperformance | 0.92 | 77% | 42% | 0.97 | 0.42 | 0.92 | 79% | 42% |
| SG43RGP46-RGPperformance | 0.97 | 87% | 25% | 0.98 | 0.25 | 0.97 | 88% | 25% |
| SG42RGP21-RGPminimalist | 0.78 | 63% | 71% | 0.95 | 0.71 | 0.78 | 65% | 71% |
| SG42RGP21-RGPminimalist | 0.88 | 78% | 66% | 0.97 | 0.66 | 0.88 | 80% | 66% |
| SG42RGP21-RGPminimalist | 0.92 | 82% | 54% | 0.98 | 0.54 | 0.92 | 84% | 54% |
| SG42RGP21-RGPminimalist | 0.94 | 81% | 42% | 0.97 | 0.42 | 0.94 | 82% | 42% |
| SG42RGP21-RGPminimalist | 1.00 | 100% | 25% | 1.00 | 0.25 | 1.00 | 100% | 25% |
| SG43RGP37-RGPconnectivity | 0.78 | 64% | 75% | 0.95 | 0.75 | 0.78 | 67% | 75% |
| SG43RGP37-RGPconnectivity | 0.87 | 76% | 68% | 0.97 | 0.68 | 0.87 | 78% | 68% |
| SG43RGP37-RGPconnectivity | 0.94 | 84% | 53% | 0.98 | 0.53 | 0.94 | 86% | 53% |
| SG43RGP37-RGPconnectivity | 0.95 | 85% | 46% | 0.98 | 0.46 | 0.95 | 87% | 46% |
| SG43RGP37-RGPconnectivity | 0.97 | 87% | 25% | 0.98 | 0.25 | 0.97 | 88% | 25% |
| SG43RGP51-PRGPminranksort | 0.83 | 72% | 75% | 0.96 | 0.75 | 0.83 | 74% | 75% |
| SG43RGP51-PRGPminranksort | 0.90 | 79% | 63% | 0.97 | 0.63 | 0.90 | 81% | 63% |
| SG43RGP51-PRGPminranksort | 0.94 | 84% | 51% | 0.98 | 0.51 | 0.94 | 85% | 51% |
| SG43RGP51-PRGPminranksort | 0.96 | 88% | 41% | 0.98 | 0.41 | 0.96 | 89% | 41% |
| SG43RGP51-PRGPminranksort | 1.00 | 100% | 10% | 1.00 | 0.10 | 1.00 | 100% | 10% |
| SG43RGP55-PRGPmedranksort | 0.81 | 69% | 76% | 0.96 | 0.76 | 0.81 | 71% | 76% |
| SG43RGP55-PRGPmedranksort | 0.86 | 72% | 64% | 0.96 | 0.64 | 0.86 | 75% | 64% |
| SG43RGP55-PRGPmedranksort | 0.92 | 81% | 51% | 0.97 | 0.51 | 0.92 | 82% | 51% |
| SG43RGP55-PRGPmedranksort | 0.95 | 82% | 37% | 0.98 | 0.37 | 0.95 | 84% | 37% |
| SG43RGP55-PRGPmedranksort | 1.00 | 100% | 15% | 1.00 | 0.15 | 1.00 | 100% | 15% |
| SG43RGP36-RGPgreedysearch | 0.83 | 76% | 86% | 0.97 | 0.86 | 0.83 | 78% | 86% |
| SG43RGP36-RGPgreedysearch | 0.94 | 89% | 80% | 0.99 | 0.80 | 0.94 | 91% | 80% |
| SG43RGP36-RGPgreedysearch | 0.96 | 90% | 53% | 0.99 | 0.53 | 0.96 | 91% | 53% |
| SG43RGP36-RGPgreedysearch | 0.97 | 87% | 25% | 0.98 | 0.25 | 0.97 | 88% | 25% |
| SG43RGP36-RGPgreedysearch | 1.00 | 100% | 5% | 1.00 | 0.05 | 1.00 | 100% | 5% |
| SG21RGP28-RGPmaxgreedysearch | 0.91 | 86% | 83% | 0.98 | 0.83 | 0.91 | 87% | 83% |
| SG21RGP28-RGPmaxgreedysearch | 0.96 | 94% | 83% | 0.99 | 0.83 | 0.96 | 95% | 83% |
| SG21RGP28-RGPmaxgreedysearch | 0.96 | 89% | 47% | 0.99 | 0.47 | 0.96 | 90% | 47% |
| SG21RGP28-RGPmaxgreedysearch | 0.97 | 87% | 25% | 0.98 | 0.25 | 0.97 | 88% | 25% |
| SG21RGP28-RGPmaxgreedysearch | 0.99 | 69% | 5% | 0.96 | 0.05 | 0.99 | 71% | 5% |

TABLE 23
Projected gains in GVHD outcome reduction and GVHD N donor capture for acute grades
II, III or IV GVHD (“VmodMedGainGag2”), assuming prevalences ranging from 35% to 55%

| | | Alternative prevalences for Gag3 | | | | | | |
|---|---|---|---|---|---|---|---|---|
| | | | 55% | 55% | | 55% | | 45% |
| | | | TNR Gneg | TPR Gneg | 55% | GVHD N | | TNR Gneg |
| | GNOS | 55% | vs. Gag2 | vs. Gag2 | GVHD | donor | 45% | vs. Gag2 |
| | thresh- | NPV Gneg | (spec- | (sensi- | reduction | capture | NPV Gneg | (spec- |
| Vmod | old | vs. Gag2 | ificity) | tivity) | for Gag2 | of Gneg | vs. Gag2 | ificity) |
| SG43RGP46-RGPperformance | 0.50 | 0.71 | 0.78 | 0.75 | 48% | 78% | 0.79 | 0.78 |
| SG43RGP46-RGPperformance | 0.55 | 0.68 | 0.66 | 0.75 | 42% | 66% | 0.76 | 0.66 |
| SG43RGP46-RGPperformance | 0.65 | 0.74 | 0.54 | 0.85 | 53% | 54% | 0.81 | 0.54 |
| SG43RGP46-RGPperformance | 0.75 | 0.73 | 0.42 | 0.87 | 51% | 42% | 0.80 | 0.42 |
| SG43RGP46-RGPperformance | 0.85 | 0.77 | 0.25 | 0.94 | 57% | 25% | 0.83 | 0.25 |
| SG42RGP21-RGPminimalist | 0.50 | 0.68 | 0.71 | 0.73 | 42% | 71% | 0.76 | 0.71 |
| SG42RGP21-RGPminimalist | 0.55 | 0.73 | 0.66 | 0.80 | 51% | 66% | 0.80 | 0.66 |
| SG42RGP21-RGPminimalist | 0.65 | 0.75 | 0.54 | 0.85 | 55% | 54% | 0.82 | 0.54 |
| SG42RGP21-RGPminimalist | 0.75 | 0.76 | 0.42 | 0.89 | 56% | 42% | 0.83 | 0.42 |
| SG42RGP21-RGPminimalist | 0.85 | 0.88 | 0.25 | 0.97 | 79% | 25% | 0.92 | 0.25 |
| SG43RGP37-RGPconnectivity | 0.50 | 0.69 | 0.75 | 0.73 | 44% | 75% | 0.77 | 0.75 |
| SG43RGP37-RGPconnectivity | 0.55 | 0.73 | 0.68 | 0.79 | 50% | 68% | 0.80 | 0.68 |
| SG43RGP37-RGPconnectivity | 0.65 | 0.75 | 0.53 | 0.85 | 54% | 53% | 0.82 | 0.53 |
| SG43RGP37-RGPconnectivity | 0.75 | 0.77 | 0.46 | 0.89 | 59% | 46% | 0.84 | 0.46 |
| SG43RGP37-RGPconnectivity | 0.85 | 0.74 | 0.25 | 0.93 | 53% | 25% | 0.81 | 0.25 |
| SG43RGP51-PRGPminranksort | 0.50 | 0.73 | 0.75 | 0.77 | 51% | 75% | 0.80 | 0.75 |
| SG43RGP51-PRGPminranksort | 0.55 | 0.75 | 0.63 | 0.83 | 54% | 63% | 0.82 | 0.63 |
| SG43RGP51-PRGPminranksort | 0.65 | 0.75 | 0.51 | 0.86 | 55% | 51% | 0.82 | 0.51 |
| SG43RGP51-PRGPminranksort | 0.75 | 0.82 | 0.41 | 0.93 | 67% | 41% | 0.87 | 0.41 |
| SG43RGP51-PRGPminranksort | 0.85 | 0.75 | 0.10 | 0.97 | 55% | 10% | 0.82 | 0.10 |
| SG43RGP55-PRGPmedranksort | 0.50 | 0.71 | 0.76 | 0.75 | 47% | 76% | 0.79 | 0.76 |
| SG43RGP55-PRGPmedranksort | 0.55 | 0.72 | 0.64 | 0.79 | 48% | 64% | 0.79 | 0.64 |
| SG43RGP55-PRGPmedranksort | 0.65 | 0.74 | 0.51 | 0.85 | 53% | 51% | 0.81 | 0.51 |
| SG43RGP55-PRGPmedranksort | 0.75 | 0.75 | 0.37 | 0.90 | 55% | 37% | 0.82 | 0.37 |
| SG43RGP55-PRGPmedranksort | 0.85 | 0.77 | 0.15 | 0.96 | 59% | 15% | 0.84 | 0.15 |
| SG43RGP36-RGPgreedysearch | 0.50 | 0.76 | 0.86 | 0.77 | 56% | 86% | 0.82 | 0.86 |
| SG43RGP36-RGPgreedysearch | 0.55 | 0.81 | 0.80 | 0.85 | 65% | 80% | 0.86 | 0.80 |
| SG43RGP36-RGPgreedysearch | 0.65 | 0.80 | 0.53 | 0.89 | 63% | 53% | 0.85 | 0.53 |
| SG43RGP36-RGPgreedysearch | 0.75 | 0.82 | 0.25 | 0.95 | 67% | 25% | 0.87 | 0.25 |
| SG43RGP36-RGPgreedysearch | 0.85 | 0.82 | 0.05 | 0.99 | 67% | 5% | 0.87 | 0.05 |
| SG21RGP28-RGPmaxgreedysearch | 0.50 | 0.79 | 0.83 | 0.82 | 62% | 83% | 0.85 | 0.83 |
| SG21RGP28-RGPmaxgreedysearch | 0.55 | 0.84 | 0.83 | 0.87 | 71% | 83% | 0.89 | 0.83 |
| SG21RGP28-RGPmaxgreedysearch | 0.65 | 0.75 | 0.47 | 0.87 | 55% | 47% | 0.82 | 0.47 |
| SG21RGP28-RGPmaxgreedysearch | 0.75 | 0.82 | 0.25 | 0.95 | 67% | 25% | 0.87 | 0.25 |
| SG21RGP28-RGPmaxgreedysearch | 0.85 | 0.53 | 0.05 | 0.96 | 15% | 5% | 0.63 | 0.05 |

| | Alternative prevalences for Gag3 | | | | | | | |
|---|---|---|---|---|---|---|---|---|
| | 45% | | 45% | | 35% | 35% | | 35% |
| | TPR Gneg | 45% | GVHD N | | TNR Gneg | TPR Gneg | 35% | GVHD N |
| | vs. Gag2 | GVHD | donor | 35% | vs. Gag2 | vs. Gag2 | GVHD | donor |
| | (sensi- | reduction | capture | NPV Gneg | (spec- | (sensi- | reduction | capture |
| Vmod | tivity) | for Gag2 | of Gneg | vs. Gag2 | ificity) | tivity) | for Gag2 | of Gneg |
| SG43RGP46-RGPperformance | 0.75 | 53% | 78% | 0.85 | 0.78 | 0.75 | 57% | 78% |
| SG43RGP46-RGPperformance | 0.75 | 47% | 66% | 0.83 | 0.66 | 0.75 | 51% | 66% |
| SG43RGP46-RGPperformance | 0.85 | 58% | 54% | 0.87 | 0.54 | 0.85 | 62% | 54% |
| SG43RGP46-RGPperformance | 0.87 | 56% | 42% | 0.86 | 0.42 | 0.87 | 60% | 42% |
| SG43RGP46-RGPperformance | 0.94 | 62% | 25% | 0.88 | 0.25 | 0.94 | 66% | 25% |
| SG42RGP21-RGPminimalist | 0.73 | 47% | 71% | 0.83 | 0.71 | 0.73 | 51% | 71% |
| SG42RGP21-RGPminimalist | 0.80 | 56% | 66% | 0.86 | 0.66 | 0.80 | 60% | 66% |
| SG42RGP21-RGPminimalist | 0.85 | 60% | 54% | 0.87 | 0.54 | 0.85 | 64% | 54% |
| SG42RGP21-RGPminimalist | 0.89 | 61% | 42% | 0.88 | 0.42 | 0.89 | 65% | 42% |
| SG42RGP21-RGPminimalist | 0.97 | 82% | 25% | 0.95 | 0.25 | 0.97 | 84% | 25% |
| SG43RGP37-RGPconnectivity | 0.73 | 49% | 75% | 0.84 | 0.75 | 0.73 | 53% | 75% |
| SG43RGP37-RGPconnectivity | 0.79 | 55% | 68% | 0.86 | 0.68 | 0.79 | 59% | 68% |
| SG43RGP37-RGPconnectivity | 0.85 | 59% | 53% | 0.87 | 0.53 | 0.85 | 63% | 53% |
| SG43RGP37-RGPconnectivity | 0.89 | 64% | 46% | 0.89 | 0.46 | 0.89 | 67% | 46% |
| SG43RGP37-RGPconnectivity | 0.93 | 58% | 25% | 0.87 | 0.25 | 0.93 | 62% | 25% |
| SG43RGP51-PRGPminranksort | 0.77 | 56% | 75% | 0.86 | 0.75 | 0.77 | 60% | 75% |
| SG43RGP51-PRGPminranksort | 0.83 | 59% | 63% | 0.87 | 0.63 | 0.83 | 63% | 63% |
| SG43RGP51-PRGPminranksort | 0.86 | 60% | 51% | 0.87 | 0.51 | 0.86 | 64% | 51% |
| SG43RGP51-PRGPminranksort | 0.93 | 72% | 41% | 0.91 | 0.41 | 0.93 | 75% | 41% |
| SG43RGP51-PRGPminranksort | 0.97 | 60% | 10% | 0.87 | 0.10 | 0.97 | 64% | 10% |
| SG43RGP55-PRGPmedranksort | 0.75 | 52% | 76% | 0.85 | 0.76 | 0.75 | 56% | 76% |
| SG43RGP55-PRGPmedranksort | 0.79 | 53% | 64% | 0.85 | 0.64 | 0.79 | 57% | 64% |
| SG43RGP55-PRGPmedranksort | 0.85 | 58% | 51% | 0.87 | 0.51 | 0.85 | 62% | 51% |
| SG43RGP55-PRGPmedranksort | 0.90 | 60% | 37% | 0.87 | 0.37 | 0.90 | 64% | 37% |
| SG43RGP55-PRGPmedranksort | 0.96 | 64% | 15% | 0.89 | 0.15 | 0.96 | 67% | 15% |
| SG43RGP36-RGPgreedysearch | 0.77 | 61% | 86% | 0.88 | 0.86 | 0.77 | 65% | 86% |
| SG43RGP36-RGPgreedysearch | 0.85 | 70% | 80% | 0.91 | 0.80 | 0.85 | 73% | 80% |
| SG43RGP36-RGPgreedysearch | 0.89 | 68% | 53% | 0.90 | 0.53 | 0.89 | 71% | 53% |
| SG43RGP36-RGPgreedysearch | 0.95 | 72% | 25% | 0.91 | 0.25 | 0.95 | 75% | 25% |
| SG43RGP36-RGPgreedysearch | 0.99 | 72% | 5% | 0.91 | 0.05 | 0.99 | 75% | 5% |
| SG21RGP28-RGPmaxgreedysearch | 0.82 | 66% | 83% | 0.89 | 0.83 | 0.82 | 70% | 83% |
| SG21RGP28-RGPmaxgreedysearch | 0.87 | 75% | 83% | 0.92 | 0.83 | 0.87 | 78% | 83% |
| SG21RGP28-RGPmaxgreedysearch | 0.87 | 60% | 47% | 0.87 | 0.47 | 0.87 | 64% | 47% |
| SG21RGP28-RGPmaxgreedysearch | 0.95 | 72% | 25% | 0.91 | 0.25 | 0.95 | 75% | 25% |
| SG21RGP28-RGPmaxgreedysearch | 0.96 | 18% | 5% | 0.72 | 0.05 | 0.96 | 21% | 5% |

Details of Outcome Predictive Performance Determination for Vmod SG43RGP36-RGPgreedysearch:

Tables 24 and 25, SG43RGP36exampleGneg and SG43RGP36exampleGag3, respectively, illustrate for Vmod SG43RGP36-RGPgreedysearch, for all Gneg and Gag3 samples and 36 RGPs the individual RGP votes for GVHD N outcomes, the GNOS value for each sample, and the final GVHD N outcome prediction for GNOS>=0.55.

As shown in Table 24 (SG43RGP36exampleGneg), a total of 47 of the 59 Gneg samples are classified correctly, i.e., are classified as true-negatives (TNs). Thus, the specificity or true negative rate, TN/(TN+FP), is 0.80 (0.7966). As shown in Table 25 (SG43RGP36exampleGag3), a total of 72 of the 77 Gag3 samples were classified correctly, i.e., classified as TPs. Thus, the sensitivity or true positive rate, TP/(TP+FN), is 0.94 (0.9350).

Assuming a prevalence of 25% for Gag3 (midpoint between the commonly accepted estimates of 15% to 35%) and given
(1) the specificity (TNR) above, the overall fraction of TNs would be 59.8% (0.7966*75%), the fraction of FPs would be 15.2% (75%−59.8%), and
(2) the sensitivity (TPR) above, the overall fraction of TPs would be 23.4% (0.9350*25%), and the fraction of FNs would be 1.6% (25%−23.4%), and
(3) therefore the negative predictive value, TN/(TN+FN), would be 0.97 (0.974; i.e., 0.598/(0.598+0.016)).

As a result of using the GVHD outcome prediction test, if only GVHD N classified donors were to be used for transplantation, 97% of transplantations would not experience acute grade III or IV GVHD, compared to 75% without using the predictive analysis. Conversely, without using the GVHD outcome prediction test, 25% of transplantations would be expected to result in acute grade III or IV GVHD, compared to 3% when using the test to select GVHD N donors. In other words, 12% of acute grade III or IV GVHD outcomes would likely still be remaining after using the GVHD outcome prediction test, but usage of the test for GVHD N donor selection would be expected to reduce GVHD by 89% (see Table 22, VmodMedGainGag3, for overview and details).

Note that in both Tables 24 and 25, SG43RGP36exampleGneg and SG43RGP36exampleGag3, samples from transplantations using

(1) bone marrow (BM) and peripheral blood stem cell (PBSC) stem cell sources are represented, and

(2) HLA 9/10 and HLA 10/10 matched donor recipient pairs are represented.

Also note that BM, PBSC, HLA 9/10 and HLA 10/10 samples, by visual inspection, are essentially evenly distributed over all the samples, whether classified as GVHD N (negative) or not, or whether classified correctly or not. In other words, the GVHD outcome prediction test correctly predicts GVHD N (negative) donors in a vast majority of cases, independently of whether the stem cell source is BM or PBSC, and irrespective of whether transplantations involve HLA 9/10 or HLA10/10 matched donor recipient pairs.

TABLE 24
Illustration for all 59 Gneg samples of Vmod SG43RGP36-RGPgreedysearch individual RGP votes, resultant
GNOS values, and final GVHD N outcome prediction for GNOS >= 0.55 (“SG43RGP36exampleGneg”)

| | | aGVHD | | | | | | | | | |
|---|---|---|---|---|---|---|---|---|---|---|---|
| Graft | HLA | outcome | cGVHD | PDCD6IP- | RPL37- | TMEM8B- | ANAPC11- | TMEM49- | MRPL42- | GINS1- | NR2F6- |
| type | match | grade | outcome | LYRM5 | GINS1 | SIPA1L2 | GINS1 | FLT3LG | GINS1 | MT1H | PRKAR1B |
| BM | 10 | 0 | 0 | 1 | 1 | 1 | 1 | 1 | 1 | 1 | 1 |
| BM | 9 | 0 | 0 | 1 | 1 | 1 | 1 | 1 | 1 | 1 | 1 |
| BM | 10 | 0 | 0 | 1 | 0 | 1 | 1 | 1 | 0 | 1 | 1 |
| BM | 10 | 0 | 0 | 1 | 1 | 1 | 1 | 1 | 1 | 1 | 1 |
| BM | 10 | 0 | 0 | 1 | 1 | 0 | 1 | 1 | 1 | 1 | 1 |
| PBSC | 10 | 0 | 0 | 0 | 1 | 1 | 0 | 1 | 1 | 1 | 1 |
| BM | 10 | 0 | 0 | 1 | 1 | 1 | 1 | 1 | 1 | 1 | 1 |
| BM | 10 | 0 | 0 | 1 | 1 | 1 | 1 | 1 | 1 | 1 | 1 |
| BM | 10 | 0 | 0 | 1 | 0 | 1 | 0 | 1 | 0 | 1 | 1 |
| BM | 10 | 0 | 0 | 1 | 1 | 1 | 1 | 1 | 1 | 1 | 1 |
| BM | 10 | 0 | 0 | 1 | 0 | 0 | 1 | 1 | 0 | 0 | 1 |
| BM | 10 | 0 | 0 | 1 | 0 | 1 | 0 | 1 | 0 | 1 | 1 |
| BM | 9 | 0 | 0 | 1 | 0 | 1 | 0 | 0 | 0 | 1 | 1 |
| BM | 10 | 0 | 0 | 1 | 1 | 1 | 1 | 0 | 1 | 1 | 1 |
| BM | 9 | 0 | 0 | 1 | 0 | 1 | 0 | 1 | 0 | 1 | 1 |
| BM | 10 | 0 | 0 | 1 | 0 | 1 | 0 | 1 | 0 | 1 | 1 |
| BM | 10 | 0 | 0 | 1 | 1 | 1 | 0 | 1 | 1 | 1 | 1 |
| BM | 10 | 0 | 0 | 1 | 1 | 1 | 0 | 1 | 1 | 0 | 1 |
| BM | 10 | 0 | 0 | 1 | 0 | 1 | 1 | 1 | 0 | 0 | 1 |
| BM | 9 | 0 | 0 | 1 | 1 | 0 | 1 | 1 | 1 | 0 | 0 |
| BM | 9 | 0 | 0 | 1 | 1 | 1 | 1 | 1 | 1 | 0 | 1 |
| BM | 10 | 0 | 0 | 0 | 1 | 0 | 1 | 0 | 1 | 1 | 1 |
| BM | 10 | 0 | 0 | 1 | 1 | 1 | 1 | 1 | 0 | 1 | 0 |
| BM | 10 | 0 | 0 | 1 | 0 | 0 | 0 | 1 | 0 | 0 | 1 |
| PBSC | 9 | 0 | 0 | 1 | 1 | 1 | 1 | 1 | 1 | 1 | 0 |
| PBSC | 10 | 0 | 0 | 0 | 0 | 1 | 0 | 1 | 0 | 1 | 1 |
| PBSC | 10 | 0 | 0 | 0 | 1 | 1 | 1 | 0 | 1 | 1 | 1 |
| BM | 9 | 0 | 0 | 0 | 1 | 0 | 1 | 1 | 1 | 0 | 1 |
| BM | 9 | 0 | 0 | 0 | 1 | 1 | 1 | 1 | 1 | 1 | 0 |
| BM | 9 | 0 | 0 | 1 | 1 | 1 | 1 | 0 | 1 | 0 | 1 |
| BM | 10 | 0 | 0 | 1 | 1 | 1 | 1 | 1 | 1 | 0 | 1 |
| BM | 10 | 0 | 0 | 1 | 0 | 0 | 0 | 1 | 0 | 0 | 1 |
| BM | 10 | 0 | 0 | 1 | 1 | 0 | 1 | 1 | 0 | 0 | 1 |
| BM | 10 | 0 | 0 | 1 | 0 | 1 | 1 | 0 | 0 | 0 | 0 |
| BM | 10 | 0 | 0 | 1 | 1 | 1 | 0 | 1 | 1 | 0 | 1 |
| BM | 9 | 0 | 0 | 0 | 1 | 1 | 1 | 1 | 1 | 1 | 1 |
| BM | 10 | 0 | 0 | 0 | 1 | 0 | 1 | 1 | 0 | 1 | 0 |
| BM | 9 | 0 | 0 | 0 | 0 | 0 | 1 | 1 | 1 | 1 | 1 |
| PBSC | 10 | 0 | 0 | 0 | 1 | 1 | 1 | 0 | 1 | 1 | 0 |
| BM | 10 | 0 | 0 | 1 | 1 | 1 | 0 | 0 | 1 | 1 | 0 |
| BM | 10 | 0 | 0 | 1 | 0 | 1 | 0 | 0 | 1 | 0 | 1 |
| BM | 9 | 0 | 0 | 0 | 1 | 0 | 1 | 0 | 1 | 0 | 1 |
| BM | 10 | 0 | 0 | 1 | 1 | 1 | 1 | 0 | 1 | 1 | 1 |
| BM | 10 | 0 | 0 | 0 | 0 | 1 | 1 | 1 | 0 | 1 | 1 |
| BM | 10 | 0 | 0 | 1 | 1 | 1 | 0 | 0 | 1 | 1 | 0 |
| BM | 10 | 0 | 0 | 0 | 1 | 0 | 1 | 1 | 1 | 1 | 1 |
| BM | 9 | 0 | 0 | 1 | 1 | 0 | 1 | 1 | 1 | 1 | 0 |
| BM | 9 | 0 | 0 | 0 | 0 | 1 | 0 | 1 | 0 | 0 | 0 |
| BM | 10 | 0 | 0 | 1 | 1 | 1 | 1 | 0 | 1 | 1 | 1 |
| BM | 9 | 0 | 0 | 0 | 0 | 0 | 1 | 1 | 0 | 0 | 0 |
| PBSC | 9 | 0 | 0 | 1 | 1 | 1 | 1 | 0 | 1 | 1 | 0 |
| BM | 10 | 0 | 0 | 0 | 1 | 0 | 1 | 1 | 1 | 0 | 0 |
| BM | 10 | 0 | 0 | 0 | 1 | 0 | 1 | 1 | 1 | 1 | 0 |
| BM | 10 | 0 | 0 | 1 | 1 | 1 | 0 | 0 | 1 | 1 | 1 |
| BM | 9 | 0 | 0 | 0 | 1 | 1 | 1 | 0 | 1 | 1 | 0 |
| BM | 10 | 0 | 0 | 1 | 1 | 0 | 1 | 0 | 1 | 1 | 0 |
| BM | 10 | 0 | 0 | 1 | 1 | 1 | 0 | 0 | 1 | 1 | 1 |
| BM | 10 | 0 | 0 | 1 | 0 | 1 | 0 | 0 | 0 | 0 | 0 |
| BM | 9 | 0 | 0 | 1 | 1 | 0 | 1 | 1 | 1 | 1 | 0 |

| Graft | MT1E- | VAMP2- | CALM3- | VAMP2- | AEBP1- | MPP5- | TMEM49- | PLAC8- | PDCD6IP- | VAMP2- | XRCC1- |
|---|---|---|---|---|---|---|---|---|---|---|---|
| type | GINS1 | TMEM5 | HEATR3 | SERPINB9 | NCDN | SEC14L1 | MRPL42 | SEC14L1 | TATDN1 | FOXN2 | SNX27 |
| BM | 1 | 0 | 1 | 1 | 1 | 1 | 1 | 1 | 1 | 0 | 1 |
| BM | 1 | 1 | 1 | 1 | 1 | 0 | 1 | 1 | 1 | 1 | 1 |
| BM | 1 | 1 | 1 | 1 | 1 | 1 | 0 | 1 | 1 | 1 | 0 |
| BM | 1 | 1 | 1 | 0 | 1 | 1 | 1 | 1 | 1 | 0 | 1 |
| BM | 1 | 0 | 0 | 1 | 1 | 1 | 1 | 1 | 1 | 1 | 1 |
| PBSC | 1 | 0 | 1 | 1 | 1 | 1 | 1 | 1 | 0 | 1 | 1 |
| BM | 0 | 1 | 1 | 1 | 1 | 1 | 1 | 1 | 1 | 0 | 1 |
| BM | 1 | 0 | 1 | 1 | 0 | 1 | 1 | 1 | 1 | 1 | 1 |
| BM | 0 | 0 | 1 | 1 | 0 | 1 | 1 | 1 | 1 | 1 | 1 |
| BM | 1 | 1 | 1 | 0 | 1 | 1 | 1 | 0 | 1 | 1 | 1 |
| BM | 1 | 1 | 1 | 1 | 0 | 1 | 1 | 1 | 1 | 1 | 1 |
| BM | 1 | 1 | 0 | 1 | 0 | 1 | 1 | 1 | 1 | 1 | 1 |
| BM | 0 | 0 | 1 | 1 | 1 | 0 | 1 | 1 | 1 | 1 | 1 |
| BM | 0 | 1 | 1 | 1 | 1 | 1 | 0 | 1 | 1 | 1 | 0 |
| BM | 1 | 1 | 1 | 1 | 1 | 1 | 1 | 1 | 0 | 1 | 1 |
| BM | 1 | 1 | 0 | 1 | 1 | 1 | 1 | 1 | 0 | 1 | 1 |
| BM | 1 | 0 | 0 | 0 | 1 | 1 | 1 | 1 | 0 | 1 | 1 |
| BM | 0 | 0 | 1 | 0 | 1 | 1 | 1 | 1 | 1 | 0 | 1 |
| BM | 1 | 1 | 1 | 1 | 0 | 1 | 1 | 1 | 1 | 1 | 1 |
| BM | 1 | 1 | 1 | 1 | 1 | 1 | 1 | 1 | 1 | 1 | 0 |
| BM | 0 | 1 | 0 | 0 | 1 | 1 | 1 | 0 | 1 | 0 | 1 |
| BM | 1 | 1 | 1 | 1 | 0 | 1 | 1 | 1 | 1 | 1 | 0 |
| BM | 1 | 1 | 1 | 1 | 0 | 0 | 1 | 0 | 1 | 1 | 1 |
| BM | 1 | 1 | 0 | 1 | 1 | 1 | 1 | 1 | 1 | 1 | 1 |
| PBSC | 1 | 1 | 1 | 1 | 1 | 0 | 0 | 0 | 0 | 1 | 1 |
| PBSC | 1 | 1 | 0 | 1 | 1 | 1 | 0 | 1 | 0 | 1 | 1 |
| PBSC | 1 | 1 | 1 | 1 | 1 | 1 | 0 | 1 | 1 | 1 | 0 |
| BM | 0 | 0 | 1 | 0 | 1 | 1 | 0 | 1 | 0 | 1 | 0 |
| BM | 0 | 1 | 1 | 1 | 1 | 1 | 0 | 0 | 0 | 1 | 1 |
| BM | 1 | 1 | 0 | 0 | 1 | 1 | 1 | 0 | 1 | 0 | 0 |
| BM | 0 | 1 | 1 | 1 | 0 | 1 | 1 | 1 | 1 | 0 | 0 |
| BM | 0 | 1 | 1 | 1 | 1 | 1 | 1 | 1 | 1 | 0 | 1 |
| BM | 0 | 0 | 1 | 1 | 1 | 1 | 1 | 1 | 1 | 1 | 0 |
| BM | 1 | 1 | 0 | 1 | 0 | 0 | 0 | 1 | 1 | 1 | 0 |
| BM | 0 | 0 | 1 | 0 | 1 | 1 | 1 | 1 | 1 | 0 | 1 |
| BM | 1 | 0 | 1 | 1 | 0 | 0 | 1 | 0 | 1 | 0 | 1 |
| BM | 1 | 0 | 0 | 1 | 1 | 1 | 0 | 1 | 1 | 1 | 1 |
| BM | 1 | 1 | 1 | 1 | 0 | 0 | 1 | 0 | 0 | 1 | 1 |
| PBSC | 1 | 1 | 1 | 1 | 0 | 0 | 0 | 0 | 0 | 1 | 1 |
| BM | 1 | 1 | 0 | 0 | 1 | 0 | 0 | 0 | 1 | 0 | 1 |
| BM | 0 | 0 | 1 | 1 | 1 | 1 | 1 | 1 | 1 | 0 | 0 |
| BM | 0 | 0 | 0 | 1 | 0 | 1 | 1 | 1 | 1 | 0 | 1 |
| BM | 1 | 1 | 0 | 1 | 0 | 1 | 1 | 1 | 0 | 1 | 0 |
| BM | 1 | 0 | 1 | 1 | 0 | 0 | 1 | 1 | 0 | 1 | 1 |
| BM | 1 | 1 | 1 | 1 | 1 | 0 | 0 | 0 | 0 | 1 | 0 |
| BM | 1 | 1 | 1 | 0 | 0 | 1 | 1 | 1 | 0 | 1 | 0 |
| BM | 1 | 1 | 1 | 1 | 1 | 0 | 0 | 0 | 0 | 1 | 1 |
| BM | 0 | 1 | 1 | 0 | 1 | 1 | 1 | 1 | 1 | 0 | 1 |
| BM | 1 | 1 | 0 | 0 | 0 | 1 | 0 | 1 | 1 | 1 | 0 |
| BM | 0 | 1 | 0 | 1 | 1 | 0 | 0 | 0 | 0 | 1 | 0 |
| PBSC | 1 | 1 | 0 | 0 | 1 | 0 | 0 | 0 | 1 | 1 | 1 |
| BM | 0 | 1 | 1 | 0 | 0 | 0 | 1 | 0 | 0 | 1 | 1 |
| BM | 1 | 0 | 0 | 1 | 0 | 0 | 1 | 0 | 1 | 0 | 0 |
| BM | 1 | 1 | 1 | 0 | 0 | 0 | 0 | 0 | 0 | 0 | 0 |
| BM | 0 | 1 | 0 | 0 | 1 | 0 | 0 | 1 | 0 | 0 | 1 |
| BM | 1 | 1 | 1 | 0 | 1 | 1 | 0 | 0 | 1 | 0 | 0 |
| BM | 0 | 0 | 1 | 0 | 1 | 0 | 1 | 0 | 0 | 0 | 1 |
| BM | 0 | 0 | 0 | 0 | 1 | 0 | 0 | 0 | 0 | 0 | 0 |
| BM | 1 | 0 | 0 | 0 | 0 | 0 | 0 | 0 | 1 | 0 | 0 |

| Graft | TMEM49- | ADRB2- | NCOA4- | FOXN2- | AEBP1- | PREX1- | AEBP1- | ABHD12- | AEBP1- | TMEM49- |
|---|---|---|---|---|---|---|---|---|---|---|
| type | CALM3 | MT1E | PAIP2 | SNURF | RPUSD1 | SMARCB1 | SARM1 | CALM3 | ZFAND5 | TATDN1 |
| BM | 1 | 1 | 1 | 1 | 1 | 1 | 1 | 1 | 1 | 1 |
| BM | 1 | 1 | 1 | 1 | 1 | 0 | 1 | 0 | 1 | 1 |
| BM | 1 | 1 | 1 | 1 | 1 | 1 | 1 | 1 | 1 | 1 |
| BM | 0 | 1 | 0 | 0 | 1 | 1 | 1 | 1 | 1 | 1 |
| BM | 0 | 0 | 1 | 1 | 1 | 1 | 1 | 1 | 1 | 1 |
| PBSC | 1 | 1 | 1 | 1 | 1 | 1 | 1 | 1 | 1 | 1 |
| BM | 0 | 0 | 0 | 1 | 1 | 1 | 0 | 0 | 1 | 1 |
| BM | 0 | 1 | 1 | 1 | 0 | 1 | 1 | 1 | 0 | 1 |
| BM | 0 | 1 | 1 | 1 | 1 | 1 | 1 | 1 | 1 | 1 |
| BM | 0 | 1 | 1 | 1 | 1 | 1 | 0 | 0 | 0 | 1 |
| BM | 1 | 1 | 1 | 1 | 0 | 1 | 1 | 1 | 0 | 1 |
| BM | 1 | 1 | 1 | 1 | 1 | 1 | 1 | 1 | 1 | 1 |
| BM | 1 | 1 | 0 | 1 | 1 | 1 | 1 | 1 | 1 | 1 |
| BM | 0 | 0 | 1 | 1 | 1 | 0 | 1 | 1 | 1 | 1 |
| BM | 1 | 1 | 1 | 1 | 1 | 1 | 1 | 1 | 1 | 0 |
| BM | 0 | 1 | 1 | 1 | 1 | 1 | 1 | 1 | 1 | 0 |
| BM | 1 | 1 | 1 | 1 | 1 | 0 | 0 | 1 | 1 | 1 |
| BM | 1 | 0 | 1 | 1 | 1 | 0 | 1 | 1 | 1 | 1 |
| BM | 1 | 1 | 1 | 1 | 0 | 1 | 1 | 1 | 0 | 1 |
| BM | 0 | 1 | 1 | 0 | 1 | 1 | 1 | 0 | 0 | 1 |
| BM | 1 | 0 | 1 | 0 | 1 | 1 | 1 | 0 | 1 | 1 |
| BM | 0 | 1 | 0 | 0 | 1 | 0 | 0 | 1 | 1 | 1 |
| BM | 1 | 1 | 1 | 1 | 0 | 0 | 0 | 1 | 0 | 1 |
| BM | 1 | 1 | 1 | 1 | 1 | 1 | 1 | 1 | 1 | 1 |
| PBSC | 1 | 1 | 1 | 0 | 1 | 0 | 1 | 1 | 1 | 0 |
| PBSC | 0 | 1 | 0 | 1 | 1 | 0 | 1 | 1 | 1 | 0 |
| PBSC | 1 | 1 | 0 | 0 | 1 | 0 | 1 | 1 | 1 | 1 |
| BM | 1 | 0 | 1 | 1 | 1 | 1 | 1 | 1 | 1 | 0 |
| BM | 1 | 0 | 1 | 1 | 1 | 1 | 1 | 1 | 1 | 0 |
| BM | 1 | 1 | 1 | 1 | 1 | 0 | 0 | 0 | 1 | 1 |
| BM | 1 | 0 | 1 | 0 | 0 | 1 | 0 | 1 | 0 | 1 |
| BM | 0 | 0 | 0 | 1 | 1 | 1 | 1 | 1 | 1 | 1 |
| BM | 0 | 0 | 1 | 1 | 0 | 1 | 1 | 0 | 1 | 0 |
| BM | 1 | 1 | 0 | 1 | 0 | 0 | 0 | 1 | 1 | 1 |
| BM | 1 | 0 | 1 | 0 | 1 | 1 | 1 | 0 | 1 | 1 |
| BM | 1 | 1 | 1 | 0 | 0 | 1 | 0 | 1 | 0 | 1 |
| BM | 1 | 0 | 1 | 1 | 1 | 1 | 0 | 1 | 0 | 1 |
| BM | 1 | 0 | 0 | 1 | 1 | 1 | 0 | 1 | 0 | 0 |
| PBSC | 0 | 1 | 1 | 1 | 0 | 0 | 0 | 1 | 0 | 0 |
| BM | 1 | 1 | 1 | 0 | 1 | 0 | 1 | 0 | 1 | 0 |
| BM | 0 | 0 | 0 | 1 | 0 | 1 | 1 | 0 | 1 | 1 |
| BM | 1 | 0 | 0 | 0 | 0 | 1 | 1 | 1 | 1 | 1 |
| BM | 0 | 1 | 1 | 0 | 0 | 1 | 0 | 0 | 0 | 1 |
| BM | 1 | 1 | 1 | 1 | 0 | 1 | 0 | 1 | 0 | 0 |
| BM | 0 | 1 | 1 | 1 | 0 | 1 | 1 | 0 | 0 | 0 |
| BM | 1 | 1 | 0 | 1 | 0 | 0 | 0 | 1 | 0 | 0 |
| BM | 1 | 1 | 1 | 0 | 0 | 0 | 1 | 0 | 0 | 0 |
| BM | 1 | 1 | 0 | 0 | 1 | 1 | 1 | 0 | 1 | 1 |
| BM | 0 | 1 | 0 | 0 | 0 | 1 | 0 | 0 | 0 | 0 |
| BM | 1 | 0 | 0 | 1 | 1 | 0 | 1 | 1 | 1 | 1 |
| PBSC | 0 | 1 | 0 | 0 | 1 | 1 | 1 | 0 | 0 | 0 |
| BM | 1 | 0 | 1 | 0 | 1 | 1 | 1 | 1 | 0 | 0 |
| BM | 1 | 0 | 0 | 0 | 0 | 1 | 0 | 0 | 1 | 1 |
| BM | 0 | 1 | 0 | 0 | 0 | 0 | 0 | 0 | 0 | 0 |
| BM | 0 | 0 | 0 | 0 | 0 | 0 | 0 | 0 | 0 | 0 |
| BM | 0 | 0 | 0 | 0 | 1 | 0 | 0 | 0 | 0 | 0 |
| BM | 1 | 0 | 0 | 0 | 0 | 0 | 0 | 0 | 0 | 0 |
| BM | 0 | 0 | 1 | 1 | 1 | 0 | 0 | 0 | 1 | 0 |
| BM | 0 | 1 | 0 | 0 | 0 | 0 | 0 | 0 | 0 | 0 |

| | | | | | | | | | GVHD N |
|---|---|---|---|---|---|---|---|---|---|
| Graft | TMEM8B- | TMEM8B- | MRPL42- | TMEM8B- | TMEM8B- | MRPL42- | AEBP1- | | vote for |
| type | C5orf62 | C16orf53 | CCDC6 | TM9SF1 | NSUN5 | FOXN2 | SEC14L1 | GNOS | GNOS >= 0.55 |
| BM | 1 | 1 | 1 | 1 | 1 | 1 | 1 | 0.94 | 1 |
| BM | 1 | 1 | 1 | 1 | 1 | 0 | 1 | 0.89 | 1 |
| BM | 1 | 1 | 1 | 1 | 1 | 0 | 1 | 0.86 | 1 |
| BM | 1 | 1 | 1 | 1 | 1 | 0 | 1 | 0.83 | 1 |
| BM | 1 | 1 | 1 | 1 | 0 | 1 | 1 | 0.83 | 1 |
| PBSC | 1 | 0 | 1 | 0 | 1 | 1 | 1 | 0.83 | 1 |
| BM | 1 | 1 | 1 | 1 | 1 | 1 | 1 | 0.81 | 1 |
| BM | 1 | 0 | 1 | 0 | 1 | 1 | 1 | 0.81 | 1 |
| BM | 1 | 1 | 1 | 1 | 1 | 1 | 1 | 0.81 | 1 |
| BM | 1 | 1 | 1 | 1 | 1 | 1 | 0 | 0.81 | 1 |
| BM | 1 | 1 | 1 | 1 | 1 | 1 | 1 | 0.81 | 1 |
| BM | 1 | 0 | 1 | 0 | 0 | 1 | 1 | 0.78 | 1 |
| BM | 0 | 1 | 1 | 1 | 1 | 1 | 1 | 0.75 | 1 |
| BM | 1 | 1 | 0 | 1 | 1 | 0 | 1 | 0.75 | 1 |
| BM | 0 | 1 | 0 | 0 | 0 | 1 | 1 | 0.75 | 1 |
| BM | 1 | 0 | 1 | 0 | 0 | 1 | 1 | 0.72 | 1 |
| BM | 1 | 0 | 1 | 0 | 0 | 1 | 1 | 0.72 | 1 |
| BM | 1 | 0 | 1 | 1 | 0 | 1 | 1 | 0.72 | 1 |
| BM | 0 | 1 | 1 | 0 | 0 | 1 | 0 | 0.72 | 1 |
| BM | 0 | 1 | 1 | 1 | 0 | 1 | 1 | 0.72 | 1 |
| BM | 1 | 1 | 0 | 1 | 1 | 0 | 1 | 0.69 | 1 |
| BM | 1 | 1 | 1 | 1 | 0 | 1 | 1 | 0.69 | 1 |
| BM | 1 | 1 | 0 | 1 | 1 | 1 | 0 | 0.69 | 1 |
| BM | 0 | 0 | 0 | 0 | 0 | 1 | 1 | 0.69 | 1 |
| PBSC | 1 | 1 | 0 | 0 | 1 | 0 | 1 | 0.69 | 1 |
| PBSC | 1 | 1 | 1 | 0 | 1 | 1 | 1 | 0.67 | 1 |
| PBSC | 0 | 0 | 0 | 1 | 0 | 0 | 1 | 0.67 | 1 |
| BM | 1 | 0 | 1 | 1 | 1 | 1 | 1 | 0.67 | 1 |
| BM | 0 | 0 | 0 | 1 | 0 | 1 | 1 | 0.67 | 1 |
| BM | 1 | 1 | 0 | 1 | 1 | 0 | 1 | 0.67 | 1 |
| BM | 1 | 1 | 1 | 1 | 0 | 1 | 0 | 0.67 | 1 |
| BM | 1 | 0 | 1 | 0 | 0 | 1 | 1 | 0.64 | 1 |
| BM | 0 | 0 | 1 | 1 | 1 | 1 | 1 | 0.64 | 1 |
| BM | 1 | 1 | 1 | 1 | 1 | 1 | 1 | 0.61 | 1 |
| BM | 0 | 0 | 1 | 0 | 0 | 0 | 1 | 0.61 | 1 |
| BM | 1 | 1 | 0 | 0 | 0 | 0 | 1 | 0.61 | 1 |
| BM | 0 | 1 | 0 | 1 | 0 | 0 | 1 | 0.61 | 1 |
| BM | 1 | 1 | 1 | 0 | 1 | 1 | 0 | 0.61 | 1 |
| PBSC | 1 | 1 | 1 | 1 | 1 | 1 | 0 | 0.58 | 1 |
| BM | 1 | 1 | 0 | 1 | 1 | 0 | 0 | 0.56 | 1 |
| BM | 1 | 0 | 1 | 0 | 1 | 1 | 0 | 0.56 | 1 |
| BM | 1 | 0 | 1 | 0 | 0 | 1 | 1 | 0.56 | 1 |
| BM | 0 | 0 | 0 | 0 | 1 | 1 | 0 | 0.56 | 1 |
| BM | 0 | 0 | 1 | 0 | 0 | 1 | 0 | 0.56 | 1 |
| BM | 1 | 1 | 0 | 1 | 1 | 0 | 0 | 0.56 | 1 |
| BM | 1 | 0 | 1 | 0 | 0 | 1 | 0 | 0.56 | 1 |
| BM | 0 | 1 | 0 | 1 | 1 | 0 | 0 | 0.56 | 1 |
| BM | 0 | 1 | 0 | 0 | 0 | 0 | 1 | 0.53 | 0 |
| BM | 0 | 1 | 1 | 1 | 1 | 0 | 0 | 0.53 | 0 |
| BM | 0 | 1 | 0 | 1 | 1 | 1 | 1 | 0.50 | 0 |
| PBSC | 0 | 0 | 1 | 0 | 1 | 0 | 0 | 0.50 | 0 |
| BM | 0 | 0 | 0 | 0 | 0 | 0 | 0 | 0.42 | 0 |
| BM | 0 | 0 | 0 | 0 | 0 | 0 | 0 | 0.36 | 0 |
| BM | 0 | 1 | 0 | 1 | 1 | 0 | 0 | 0.36 | 0 |
| BM | 1 | 1 | 0 | 1 | 0 | 0 | 1 | 0.36 | 0 |
| BM | 0 | 0 | 0 | 0 | 0 | 0 | 0 | 0.33 | 0 |
| BM | 0 | 0 | 0 | 0 | 0 | 0 | 0 | 0.31 | 0 |
| BM | 1 | 0 | 0 | 1 | 0 | 0 | 0 | 0.25 | 0 |
| BM | 0 | 0 | 0 | 0 | 0 | 0 | 0 | 0.25 | 0 |

TABLE 25
Illustration for all 77 Gag3 samples of Vmod SG43RGP36-RGPgreedysearch individual RGP votes, resultant
GNOS values, and final GVHD N outcome prediction for GNOS >= 0.55 (“SG43RGP36exampleGag3”)

| | | aGVHD | | | | | | | | | |
|---|---|---|---|---|---|---|---|---|---|---|---|
| Graft | HLA | outcome | cGVHD | PDCD6IP- | RPL37- | TMEM8B- | ANAPC11- | TMEM49- | MRPL42- | GINS1- | NR2F6- |
| type | match | grade | outcome | LYRM5 | GINS1 | SIPA1L2 | GINS1 | FLT3LG | GINS1 | MT1H | PRKAR1B |
| BM | 9 | 4 | 0 | 1 | 1 | 1 | 1 | 1 | 1 | 1 | 0 |
| BM | 9 | 3 | 1 | 1 | 0 | 0 | 0 | 1 | 0 | 0 | 1 |
| BM | 10 | 3 | 1 | 1 | 1 | 1 | 1 | 0 | 1 | 1 | 1 |
| BM | 10 | 3 | 0 | 1 | 1 | 1 | 1 | 1 | 1 | 1 | 0 |
| PBSC | 10 | 4 | 0 | 1 | 1 | 1 | 0 | 1 | 1 | 1 | 1 |
| PBSC | 10 | 3 | 0 | 1 | 1 | 0 | 1 | 1 | 1 | 1 | 0 |
| PBSC | 9 | 3 | 0 | 0 | 1 | 0 | 1 | 0 | 1 | 1 | 1 |
| BM | 10 | 3 | 1 | 1 | 0 | 0 | 1 | 1 | 0 | 1 | 0 |
| BM | 9 | 3 | 1 | 0 | 1 | 1 | 1 | 1 | 1 | 1 | 0 |
| PBSC | 10 | 3 | 1 | 0 | 1 | 0 | 1 | 0 | 1 | 1 | 1 |
| PBSC | 10 | 4 | 0 | 1 | 1 | 1 | 0 | 1 | 0 | 1 | 0 |
| PBSC | 10 | 3 | 1 | 0 | 1 | 0 | 1 | 1 | 1 | 1 | 0 |
| BM | 10 | 4 | 1 | 0 | 1 | 0 | 0 | 1 | 1 | 0 | 1 |
| BM | 9 | 4 | 1 | 0 | 0 | 1 | 0 | 1 | 0 | 0 | 0 |
| BM | 10 | 3 | 1 | 0 | 1 | 0 | 1 | 1 | 0 | 0 | 0 |
| BM | 10 | 4 | 1 | 0 | 0 | 1 | 0 | 0 | 0 | 1 | 0 |
| PBSC | 9 | 3 | 0 | 0 | 0 | 0 | 1 | 1 | 1 | 0 | 0 |
| BM | 9 | 3 | 1 | 0 | 0 | 0 | 0 | 0 | 0 | 0 | 1 |
| BM | 10 | 3 | 0 | 0 | 1 | 0 | 1 | 0 | 1 | 1 | 1 |
| PBSC | 10 | 3 | 1 | 0 | 0 | 0 | 0 | 1 | 0 | 0 | 1 |
| BM | 9 | 3 | 1 | 0 | 0 | 1 | 0 | 0 | 0 | 0 | 1 |
| BM | 10 | 4 | 0 | 1 | 1 | 1 | 1 | 1 | 1 | 0 | 0 |
| BM | 9 | 4 | 0 | 0 | 0 | 1 | 0 | 0 | 1 | 1 | 1 |
| BM | 10 | 3 | 1 | 1 | 1 | 1 | 1 | 0 | 1 | 1 | 1 |
| BM | 10 | 3 | 1 | 0 | 1 | 1 | 1 | 1 | 1 | 1 | 1 |
| BM | 10 | 3 | 1 | 1 | 1 | 1 | 1 | 0 | 1 | 1 | 1 |
| BM | 10 | 3 | 1 | 0 | 0 | 0 | 0 | 0 | 0 | 1 | 0 |
| BM | 10 | 3 | 0 | 0 | 0 | 1 | 0 | 1 | 0 | 0 | 0 |
| BM | 9 | 3 | 0 | 0 | 1 | 1 | 0 | 1 | 0 | 0 | 0 |
| BM | 10 | 3 | 1 | 0 | 1 | 1 | 1 | 0 | 1 | 0 | 1 |
| BM | 10 | 3 | 1 | 0 | 1 | 0 | 0 | 0 | 1 | 0 | 1 |
| BM | 9 | 4 | 0 | 0 | 0 | 0 | 0 | 1 | 0 | 0 | 0 |
| PBSC | 10 | 3 | 0 | 0 | 0 | 1 | 0 | 0 | 0 | 0 | 1 |
| BM | 10 | 3 | 0 | 0 | 1 | 0 | 1 | 0 | 1 | 0 | 1 |
| BM | 10 | 3 | 0 | 1 | 0 | 1 | 0 | 1 | 0 | 0 | 0 |
| BM | 10 | 3 | 1 | 0 | 0 | 0 | 0 | 0 | 0 | 0 | 0 |
| BM | 10 | 3 | 1 | 1 | 0 | 0 | 0 | 1 | 0 | 1 | 0 |
| BM | 10 | 3 | 1 | 1 | 1 | 1 | 1 | 0 | 1 | 0 | 0 |
| PBSC | 10 | 4 | 1 | 0 | 1 | 0 | 0 | 1 | 1 | 0 | 0 |
| BM | 9 | 3 | 0 | 0 | 0 | 0 | 1 | 1 | 1 | 0 | 0 |
| BM | 10 | 3 | 1 | 1 | 0 | 0 | 0 | 0 | 0 | 1 | 0 |
| BM | 9 | 3 | 1 | 0 | 0 | 0 | 0 | 0 | 0 | 1 | 1 |
| BM | 9 | 3 | 1 | 1 | 1 | 0 | 1 | 0 | 1 | 1 | 0 |
| BM | 9 | 4 | 1 | 0 | 0 | 0 | 0 | 0 | 0 | 0 | 0 |
| BM | 10 | 4 | 1 | 0 | 0 | 1 | 1 | 0 | 1 | 0 | 0 |
| BM | 10 | 4 | 1 | 0 | 1 | 1 | 1 | 0 | 1 | 0 | 0 |
| BM | 10 | 3 | 0 | 0 | 0 | 0 | 0 | 1 | 0 | 0 | 1 |
| BM | 10 | 3 | 0 | 0 | 0 | 0 | 0 | 0 | 0 | 0 | 0 |
| BM | 9 | 3 | 1 | 0 | 0 | 0 | 0 | 0 | 0 | 0 | 1 |
| PBSC | 10 | 3 | 1 | 0 | 0 | 0 | 1 | 0 | 1 | 1 | 0 |
| BM | 9 | 3 | 1 | 0 | 0 | 1 | 0 | 0 | 0 | 0 | 0 |
| BM | 9 | 4 | 1 | 1 | 0 | 0 | 0 | 1 | 0 | 0 | 0 |
| BM | 10 | 3 | 0 | 1 | 1 | 0 | 1 | 0 | 1 | 0 | 1 |
| BM | 10 | 3 | 0 | 0 | 0 | 1 | 0 | 0 | 0 | 0 | 1 |
| BM | 9 | 3 | 0 | 1 | 0 | 0 | 0 | 0 | 0 | 0 | 0 |
| BM | 9 | 3 | 0 | 1 | 1 | 1 | 0 | 0 | 0 | 0 | 0 |
| BM | 10 | 3 | 1 | 1 | 0 | 0 | 1 | 0 | 0 | 0 | 0 |
| BM | 10 | 3 | 1 | 0 | 0 | 0 | 1 | 0 | 1 | 0 | 0 |
| BM | 9 | 4 | 1 | 0 | 1 | 1 | 1 | 0 | 1 | 1 | 0 |
| BM | 10 | 4 | 1 | 0 | 0 | 0 | 1 | 0 | 1 | 0 | 0 |
| BM | 10 | 3 | 0 | 1 | 0 | 0 | 0 | 0 | 0 | 0 | 0 |
| BM | 9 | 4 | 0 | 0 | 0 | 0 | 0 | 0 | 0 | 0 | 1 |
| BM | 10 | 3 | 0 | 1 | 0 | 0 | 0 | 0 | 0 | 1 | 1 |
| BM | 10 | 3 | 0 | 0 | 1 | 1 | 0 | 1 | 0 | 1 | 0 |
| BM | 10 | 3 | 0 | 0 | 0 | 0 | 0 | 1 | 0 | 0 | 0 |
| BM | 10 | 3 | 1 | 0 | 0 | 0 | 0 | 0 | 0 | 0 | 0 |
| PBSC | 10 | 3 | 1 | 0 | 1 | 1 | 0 | 0 | 0 | 1 | 0 |
| BM | 10 | 3 | 1 | 0 | 0 | 0 | 0 | 0 | 0 | 1 | 0 |
| BM | 9 | 4 | 0 | 0 | 0 | 0 | 0 | 0 | 0 | 0 | 1 |
| BM | 9 | 3 | 0 | 0 | 1 | 1 | 0 | 0 | 0 | 0 | 1 |
| BM | 9 | 3 | 1 | 1 | 0 | 1 | 0 | 0 | 0 | 0 | 0 |
| BM | 10 | 4 | 1 | 0 | 0 | 0 | 0 | 0 | 0 | 1 | 0 |
| BM | 10 | 3 | 0 | 1 | 1 | 0 | 0 | 0 | 0 | 0 | 1 |
| BM | 10 | 3 | 0 | 1 | 0 | 0 | 0 | 0 | 0 | 0 | 0 |
| BM | 10 | 3 | 1 | 0 | 0 | 0 | 0 | 0 | 0 | 0 | 0 |
| BM | 10 | 3 | 1 | 0 | 0 | 0 | 0 | 0 | 0 | 0 | 0 |
| BM | 10 | 3 | 1 | 0 | 0 | 0 | 0 | 0 | 0 | 0 | 0 |

| Graft | MT1E- | VAMP2- | CALM3- | VAMP2- | AEBP1- | MPP5- | TMEM49- | PLAC8- | PDCD6IP- | VAMP2- | XRCC1- |
|---|---|---|---|---|---|---|---|---|---|---|---|
| type | GINS1 | TMEM5 | HEATR3 | SERPINB9 | NCDN | SEC14L1 | MRPL42 | SEC14L1 | TATDN1 | FOXN2 | SNX27 |
| BM | 1 | 1 | 1 | 1 | 0 | 1 | 1 | 1 | 1 | 1 | 1 |
| BM | 1 | 1 | 1 | 1 | 1 | 1 | 1 | 1 | 1 | 1 | 1 |
| BM | 1 | 1 | 1 | 0 | 1 | 1 | 0 | 1 | 1 | 1 | 0 |
| BM | 0 | 1 | 1 | 0 | 1 | 0 | 1 | 0 | 1 | 0 | 1 |
| PBSC | 1 | 1 | 1 | 1 | 1 | 0 | 1 | 0 | 0 | 1 | 1 |
| PBSC | 1 | 0 | 0 | 1 | 0 | 1 | 1 | 0 | 0 | 1 | 1 |
| PBSC | 1 | 1 | 0 | 1 | 0 | 0 | 0 | 1 | 1 | 1 | 1 |
| BM | 1 | 0 | 1 | 1 | 0 | 0 | 0 | 1 | 1 | 1 | 1 |
| BM | 1 | 0 | 1 | 0 | 1 | 1 | 0 | 0 | 0 | 1 | 0 |
| PBSC | 1 | 1 | 0 | 1 | 0 | 1 | 0 | 1 | 1 | 1 | 1 |
| PBSC | 1 | 0 | 0 | 1 | 1 | 0 | 0 | 0 | 0 | 1 | 1 |
| PBSC | 1 | 0 | 1 | 1 | 0 | 0 | 0 | 0 | 0 | 1 | 1 |
| BM | 0 | 0 | 1 | 1 | 0 | 1 | 1 | 1 | 0 | 0 | 1 |
| BM | 0 | 1 | 0 | 0 | 1 | 0 | 0 | 0 | 1 | 1 | 0 |
| BM | 0 | 0 | 0 | 1 | 0 | 1 | 0 | 1 | 1 | 1 | 1 |
| BM | 1 | 1 | 0 | 0 | 1 | 0 | 0 | 0 | 0 | 1 | 0 |
| PBSC | 0 | 1 | 1 | 1 | 0 | 0 | 1 | 1 | 1 | 1 | 0 |
| BM | 0 | 0 | 0 | 1 | 1 | 1 | 1 | 1 | 0 | 0 | 0 |
| BM | 1 | 0 | 0 | 0 | 0 | 0 | 0 | 0 | 0 | 0 | 0 |
| PBSC | 0 | 0 | 0 | 1 | 0 | 0 | 1 | 1 | 0 | 1 | 1 |
| BM | 0 | 0 | 1 | 0 | 1 | 0 | 0 | 1 | 0 | 1 | 0 |
| BM | 0 | 1 | 0 | 0 | 1 | 1 | 0 | 0 | 0 | 1 | 0 |
| BM | 1 | 1 | 0 | 1 | 1 | 0 | 1 | 0 | 0 | 0 | 0 |
| BM | 0 | 1 | 1 | 0 | 1 | 0 | 1 | 0 | 1 | 0 | 0 |
| BM | 1 | 0 | 1 | 0 | 0 | 0 | 0 | 0 | 0 | 0 | 0 |
| BM | 1 | 0 | 0 | 0 | 0 | 0 | 0 | 0 | 0 | 1 | 1 |
| BM | 1 | 1 | 0 | 0 | 0 | 1 | 1 | 0 | 0 | 0 | 0 |
| BM | 0 | 1 | 1 | 0 | 1 | 0 | 0 | 0 | 0 | 1 | 0 |
| BM | 0 | 1 | 1 | 0 | 1 | 0 | 0 | 0 | 0 | 1 | 1 |
| BM | 0 | 0 | 1 | 0 | 0 | 1 | 1 | 0 | 0 | 0 | 1 |
| BM | 1 | 0 | 0 | 1 | 1 | 0 | 0 | 1 | 0 | 0 | 0 |
| BM | 0 | 1 | 1 | 1 | 0 | 0 | 0 | 0 | 0 | 1 | 1 |
| PBSC | 1 | 1 | 0 | 1 | 0 | 1 | 0 | 0 | 0 | 0 | 0 |
| BM | 0 | 0 | 0 | 0 | 0 | 0 | 0 | 1 | 1 | 0 | 0 |
| BM | 1 | 0 | 0 | 0 | 0 | 1 | 1 | 0 | 0 | 0 | 0 |
| BM | 0 | 0 | 1 | 0 | 1 | 0 | 0 | 0 | 1 | 0 | 1 |
| BM | 0 | 1 | 0 | 0 | 0 | 0 | 1 | 0 | 0 | 0 | 0 |
| BM | 0 | 0 | 0 | 0 | 0 | 1 | 1 | 1 | 0 | 0 | 0 |
| PBSC | 0 | 0 | 0 | 1 | 0 | 0 | 1 | 0 | 0 | 1 | 1 |
| BM | 0 | 0 | 0 | 1 | 0 | 0 | 1 | 0 | 0 | 0 | 1 |
| BM | 1 | 0 | 0 | 0 | 0 | 0 | 0 | 0 | 1 | 0 | 1 |
| BM | 1 | 0 | 1 | 1 | 0 | 1 | 0 | 0 | 0 | 1 | 0 |
| BM | 1 | 0 | 0 | 0 | 0 | 0 | 0 | 0 | 0 | 0 | 0 |
| BM | 0 | 0 | 1 | 1 | 0 | 0 | 1 | 0 | 1 | 0 | 1 |
| BM | 0 | 0 | 0 | 0 | 0 | 1 | 0 | 1 | 0 | 0 | 0 |
| BM | 0 | 0 | 0 | 0 | 0 | 0 | 1 | 0 | 1 | 0 | 0 |
| BM | 0 | 0 | 1 | 0 | 0 | 0 | 0 | 0 | 0 | 0 | 0 |
| BM | 0 | 0 | 0 | 0 | 1 | 0 | 1 | 0 | 0 | 0 | 0 |
| BM | 0 | 0 | 1 | 1 | 0 | 0 | 0 | 1 | 1 | 0 | 0 |
| PBSC | 1 | 0 | 0 | 0 | 0 | 1 | 0 | 1 | 0 | 0 | 0 |
| BM | 0 | 0 | 0 | 0 | 0 | 0 | 0 | 0 | 0 | 0 | 0 |
| BM | 0 | 0 | 1 | 1 | 1 | 1 | 1 | 0 | 1 | 0 | 0 |
| BM | 0 | 1 | 0 | 0 | 1 | 0 | 0 | 0 | 0 | 0 | 1 |
| BM | 0 | 0 | 0 | 0 | 0 | 0 | 0 | 0 | 0 | 0 | 0 |
| BM | 1 | 1 | 1 | 0 | 0 | 0 | 0 | 0 | 1 | 0 | 0 |
| BM | 0 | 1 | 0 | 0 | 1 | 0 | 0 | 0 | 0 | 0 | 1 |
| BM | 0 | 0 | 0 | 1 | 0 | 0 | 1 | 0 | 0 | 0 | 0 |
| BM | 0 | 0 | 0 | 0 | 0 | 0 | 1 | 0 | 0 | 1 | 0 |
| BM | 0 | 1 | 0 | 0 | 0 | 0 | 0 | 0 | 1 | 0 | 0 |
| BM | 0 | 0 | 0 | 0 | 0 | 0 | 0 | 0 | 0 | 0 | 1 |
| BM | 1 | 1 | 0 | 0 | 0 | 0 | 1 | 0 | 0 | 0 | 1 |
| BM | 0 | 1 | 1 | 1 | 0 | 0 | 0 | 0 | 1 | 0 | 1 |
| BM | 0 | 0 | 0 | 0 | 1 | 0 | 0 | 0 | 1 | 0 | 0 |
| BM | 0 | 0 | 1 | 1 | 0 | 0 | 0 | 0 | 0 | 0 | 0 |
| BM | 0 | 0 | 0 | 0 | 0 | 0 | 0 | 0 | 0 | 1 | 0 |
| BM | 0 | 1 | 0 | 1 | 0 | 0 | 0 | 0 | 1 | 0 | 1 |
| PBSC | 1 | 0 | 1 | 0 | 0 | 0 | 0 | 0 | 0 | 0 | 0 |
| BM | 1 | 0 | 1 | 0 | 0 | 1 | 0 | 1 | 0 | 0 | 0 |
| BM | 0 | 0 | 0 | 0 | 0 | 0 | 1 | 0 | 0 | 0 | 0 |
| BM | 0 | 0 | 0 | 0 | 1 | 0 | 0 | 0 | 0 | 0 | 0 |
| BM | 0 | 0 | 0 | 0 | 0 | 0 | 0 | 0 | 0 | 0 | 0 |
| BM | 0 | 0 | 0 | 0 | 0 | 0 | 0 | 0 | 0 | 0 | 0 |
| BM | 0 | 0 | 0 | 0 | 0 | 0 | 0 | 0 | 1 | 0 | 0 |
| BM | 0 | 0 | 0 | 0 | 1 | 0 | 0 | 0 | 1 | 0 | 0 |
| BM | 0 | 0 | 0 | 0 | 0 | 0 | 0 | 0 | 0 | 0 | 1 |
| BM | 0 | 0 | 0 | 0 | 0 | 1 | 0 | 1 | 0 | 0 | 0 |
| BM | 0 | 0 | 0 | 0 | 0 | 0 | 0 | 0 | 0 | 0 | 0 |

| Graft | TMEM49- | ADRB2- | NCOA4- | FOXN2- | AEBP1- | PREX1- | AEBP1- | ABHD12- | AEBP1- | TMEM49- |
|---|---|---|---|---|---|---|---|---|---|---|
| type | CALM3 | MT1E | PAIP2 | SNURF | RPUSD1 | SMARCB1 | SARM1 | CALM3 | ZFAND5 | TATDN1 |
| BM | 1 | 1 | 1 | 0 | 0 | 1 | 0 | 1 | 1 | 1 |
| BM | 1 | 1 | 1 | 0 | 1 | 1 | 1 | 0 | 1 | 1 |
| BM | 0 | 1 | 1 | 1 | 1 | 1 | 1 | 0 | 1 | 0 |
| BM | 1 | 0 | 1 | 1 | 1 | 0 | 0 | 0 | 0 | 1 |
| PBSC | 0 | 1 | 0 | 0 | 1 | 0 | 1 | 0 | 1 | 0 |
| PBSC | 1 | 1 | 1 | 1 | 1 | 0 | 0 | 1 | 0 | 0 |
| PBSC | 0 | 1 | 0 | 0 | 0 | 1 | 0 | 1 | 0 | 1 |
| BM | 1 | 1 | 1 | 1 | 0 | 1 | 0 | 1 | 0 | 1 |
| BM | 0 | 1 | 1 | 1 | 1 | 0 | 1 | 0 | 1 | 0 |
| PBSC | 0 | 1 | 0 | 0 | 0 | 1 | 0 | 0 | 1 | 0 |
| PBSC | 0 | 1 | 1 | 1 | 1 | 0 | 1 | 0 | 0 | 1 |
| PBSC | 1 | 0 | 1 | 1 | 0 | 0 | 0 | 1 | 0 | 0 |
| BM | 1 | 0 | 1 | 0 | 1 | 0 | 1 | 0 | 1 | 0 |
| BM | 1 | 1 | 0 | 1 | 1 | 0 | 1 | 1 | 1 | 0 |
| BM | 1 | 0 | 1 | 0 | 0 | 0 | 0 | 1 | 0 | 1 |
| BM | 0 | 1 | 0 | 1 | 1 | 0 | 0 | 0 | 0 | 1 |
| PBSC | 0 | 0 | 0 | 0 | 0 | 1 | 0 | 1 | 0 | 1 |
| BM | 1 | 0 | 0 | 0 | 0 | 1 | 1 | 1 | 1 | 0 |
| BM | 0 | 1 | 1 | 1 | 0 | 1 | 0 | 1 | 0 | 0 |
| PBSC | 1 | 1 | 1 | 0 | 0 | 0 | 0 | 1 | 0 | 1 |
| BM | 0 | 0 | 0 | 0 | 1 | 1 | 1 | 1 | 1 | 0 |
| BM | 0 | 0 | 1 | 1 | 0 | 0 | 0 | 0 | 1 | 1 |
| BM | 1 | 1 | 0 | 0 | 1 | 0 | 0 | 0 | 0 | 0 |
| BM | 0 | 0 | 0 | 0 | 1 | 0 | 0 | 0 | 0 | 0 |
| BM | 1 | 0 | 1 | 1 | 0 | 0 | 0 | 1 | 0 | 0 |
| BM | 0 | 0 | 0 | 1 | 0 | 0 | 0 | 0 | 0 | 0 |
| BM | 1 | 0 | 1 | 1 | 0 | 0 | 0 | 0 | 0 | 0 |
| BM | 0 | 0 | 1 | 1 | 0 | 0 | 1 | 0 | 0 | 1 |
| BM | 1 | 0 | 0 | 0 | 0 | 0 | 1 | 0 | 0 | 0 |
| BM | 0 | 0 | 1 | 0 | 0 | 0 | 0 | 0 | 0 | 1 |
| BM | 0 | 0 | 0 | 0 | 1 | 0 | 1 | 0 | 1 | 0 |
| BM | 1 | 0 | 0 | 0 | 1 | 0 | 1 | 1 | 1 | 0 |
| PBSC | 0 | 1 | 0 | 1 | 0 | 0 | 0 | 1 | 0 | 0 |
| BM | 0 | 0 | 0 | 0 | 1 | 0 | 0 | 0 | 1 | 1 |
| BM | 0 | 1 | 1 | 1 | 0 | 0 | 0 | 0 | 0 | 1 |
| BM | 1 | 0 | 0 | 0 | 1 | 1 | 1 | 1 | 1 | 0 |
| BM | 1 | 0 | 1 | 0 | 1 | 0 | 1 | 0 | 0 | 0 |
| BM | 0 | 0 | 1 | 0 | 0 | 1 | 0 | 0 | 0 | 0 |
| PBSC | 1 | 0 | 1 | 0 | 0 | 0 | 0 | 0 | 0 | 0 |
| BM | 1 | 0 | 0 | 0 | 0 | 1 | 0 | 1 | 0 | 0 |
| BM | 0 | 1 | 0 | 0 | 1 | 1 | 1 | 1 | 0 | 0 |
| BM | 0 | 1 | 0 | 1 | 0 | 0 | 0 | 0 | 1 | 0 |
| BM | 0 | 1 | 0 | 0 | 0 | 0 | 0 | 0 | 0 | 0 |
| BM | 1 | 1 | 0 | 0 | 0 | 0 | 0 | 0 | 0 | 1 |
| BM | 0 | 0 | 0 | 0 | 0 | 0 | 0 | 0 | 0 | 0 |
| BM | 0 | 0 | 0 | 0 | 0 | 1 | 0 | 0 | 0 | 1 |
| BM | 0 | 0 | 0 | 1 | 0 | 0 | 0 | 1 | 0 | 0 |
| BM | 1 | 0 | 0 | 1 | 0 | 1 | 0 | 1 | 1 | 0 |
| BM | 0 | 1 | 0 | 0 | 0 | 1 | 1 | 0 | 0 | 1 |
| PBSC | 0 | 1 | 0 | 0 | 0 | 1 | 0 | 0 | 0 | 0 |
| BM | 0 | 0 | 0 | 0 | 1 | 0 | 1 | 1 | 0 | 0 |
| BM | 0 | 0 | 0 | 0 | 0 | 0 | 0 | 0 | 0 | 1 |
| BM | 0 | 0 | 0 | 0 | 0 | 0 | 0 | 0 | 0 | 0 |
| BM | 0 | 0 | 0 | 0 | 0 | 0 | 0 | 0 | 1 | 0 |
| BM | 0 | 1 | 0 | 0 | 0 | 0 | 0 | 0 | 0 | 0 |
| BM | 0 | 0 | 1 | 0 | 0 | 0 | 0 | 0 | 0 | 0 |
| BM | 1 | 0 | 0 | 1 | 0 | 1 | 1 | 0 | 0 | 0 |
| BM | 0 | 0 | 0 | 1 | 0 | 0 | 1 | 0 | 1 | 0 |
| BM | 0 | 0 | 1 | 0 | 0 | 0 | 0 | 0 | 0 | 0 |
| BM | 0 | 0 | 0 | 1 | 1 | 0 | 1 | 0 | 1 | 0 |
| BM | 0 | 1 | 0 | 0 | 0 | 0 | 0 | 0 | 0 | 0 |
| BM | 1 | 0 | 0 | 0 | 0 | 0 | 0 | 0 | 0 | 0 |
| BM | 0 | 0 | 0 | 0 | 1 | 0 | 0 | 0 | 0 | 0 |
| BM | 0 | 0 | 0 | 0 | 0 | 0 | 0 | 0 | 0 | 0 |
| BM | 1 | 0 | 0 | 1 | 0 | 1 | 0 | 1 | 0 | 0 |
| BM | 0 | 0 | 0 | 0 | 0 | 1 | 0 | 0 | 0 | 0 |
| PBSC | 0 | 1 | 0 | 0 | 0 | 0 | 0 | 0 | 0 | 0 |
| BM | 0 | 0 | 0 | 0 | 0 | 0 | 0 | 0 | 0 | 0 |
| BM | 1 | 0 | 0 | 0 | 0 | 1 | 0 | 0 | 0 | 0 |
| BM | 0 | 0 | 0 | 0 | 0 | 0 | 1 | 0 | 0 | 0 |
| BM | 0 | 0 | 1 | 1 | 0 | 0 | 0 | 1 | 0 | 0 |
| BM | 1 | 0 | 0 | 0 | 1 | 0 | 0 | 0 | 0 | 0 |
| BM | 0 | 0 | 0 | 0 | 0 | 0 | 0 | 0 | 0 | 0 |
| BM | 0 | 0 | 0 | 0 | 0 | 0 | 0 | 0 | 0 | 1 |
| BM | 0 | 0 | 0 | 0 | 1 | 1 | 0 | 0 | 1 | 0 |
| BM | 0 | 0 | 0 | 0 | 0 | 0 | 1 | 0 | 1 | 0 |
| BM | 0 | 0 | 0 | 0 | 0 | 0 | 0 | 0 | 0 | 0 |

| | | | | | | | | | GVHD N |
|---|---|---|---|---|---|---|---|---|---|
| Graft | TMEM8B- | TMEM8B- | MRPL42- | TMEM8B- | TMEM8B- | MRPL42- | AEBP1- | | vote for |
| type | C5orf62 | C16orf53 | CCDC6 | TM9SF1 | NSUN5 | FOXN2 | SEC14L1 | GNOS | GNOS >= 0.55 |
| BM | 1 | 1 | 1 | 1 | 0 | 1 | 1 | 0.83 | 1 |
| BM | 1 | 1 | 1 | 1 | 1 | 0 | 1 | 0.78 | 1 |
| BM | 0 | 0 | 0 | 1 | 1 | 0 | 1 | 0.69 | 1 |
| BM | 0 | 0 | 0 | 1 | 1 | 0 | 0 | 0.56 | 1 |
| PBSC | 0 | 0 | 0 | 0 | 0 | 0 | 1 | 0.56 | 1 |
| PBSC | 0 | 0 | 0 | 1 | 0 | 0 | 0 | 0.53 | 0 |
| PBSC | 0 | 1 | 1 | 0 | 0 | 1 | 0 | 0.53 | 0 |
| BM | 0 | 0 | 0 | 0 | 0 | 1 | 0 | 0.53 | 0 |
| BM | 1 | 0 | 0 | 0 | 0 | 0 | 1 | 0.53 | 0 |
| PBSC | 1 | 0 | 1 | 0 | 0 | 0 | 1 | 0.53 | 0 |
| PBSC | 0 | 0 | 0 | 1 | 1 | 1 | 0 | 0.53 | 0 |
| PBSC | 1 | 1 | 0 | 1 | 1 | 0 | 0 | 0.50 | 0 |
| BM | 1 | 0 | 1 | 0 | 0 | 0 | 1 | 0.50 | 0 |
| BM | 0 | 1 | 0 | 1 | 1 | 0 | 1 | 0.47 | 0 |
| BM | 0 | 0 | 1 | 1 | 0 | 1 | 0 | 0.44 | 0 |
| BM | 1 | 1 | 1 | 1 | 1 | 1 | 0 | 0.44 | 0 |
| PBSC | 0 | 0 | 1 | 0 | 0 | 1 | 0 | 0.42 | 0 |
| BM | 1 | 0 | 1 | 0 | 0 | 1 | 1 | 0.42 | 0 |
| BM | 1 | 1 | 0 | 0 | 0 | 1 | 0 | 0.39 | 0 |
| PBSC | 0 | 0 | 1 | 0 | 0 | 1 | 0 | 0.39 | 0 |
| BM | 1 | 0 | 0 | 1 | 0 | 0 | 1 | 0.39 | 0 |
| BM | 0 | 0 | 0 | 0 | 0 | 0 | 0 | 0.39 | 0 |
| BM | 0 | 1 | 0 | 0 | 1 | 0 | 0 | 0.39 | 0 |
| BM | 0 | 0 | 0 | 0 | 0 | 0 | 0 | 0.36 | 0 |
| BM | 0 | 0 | 0 | 0 | 0 | 0 | 0 | 0.36 | 0 |
| BM | 1 | 0 | 0 | 1 | 0 | 0 | 0 | 0.36 | 0 |
| BM | 1 | 1 | 0 | 1 | 1 | 1 | 0 | 0.36 | 0 |
| BM | 0 | 1 | 0 | 1 | 0 | 0 | 0 | 0.33 | 0 |
| BM | 1 | 0 | 0 | 0 | 1 | 0 | 0 | 0.33 | 0 |
| BM | 0 | 0 | 0 | 0 | 1 | 0 | 0 | 0.33 | 0 |
| BM | 0 | 0 | 1 | 0 | 0 | 0 | 1 | 0.33 | 0 |
| BM | 0 | 0 | 0 | 0 | 0 | 0 | 1 | 0.33 | 0 |
| PBSC | 1 | 0 | 0 | 0 | 0 | 1 | 0 | 0.31 | 0 |
| BM | 0 | 0 | 1 | 0 | 0 | 0 | 1 | 0.31 | 0 |
| BM | 0 | 0 | 1 | 0 | 0 | 0 | 0 | 0.31 | 0 |
| BM | 0 | 0 | 0 | 0 | 0 | 0 | 1 | 0.31 | 0 |
| BM | 0 | 0 | 1 | 0 | 0 | 1 | 0 | 0.31 | 0 |
| BM | 0 | 0 | 1 | 0 | 0 | 0 | 0 | 0.31 | 0 |
| PBSC | 0 | 1 | 0 | 0 | 0 | 1 | 0 | 0.31 | 0 |
| BM | 0 | 0 | 0 | 0 | 0 | 1 | 0 | 0.28 | 0 |
| BM | 0 | 0 | 0 | 0 | 0 | 0 | 0 | 0.28 | 0 |
| BM | 0 | 0 | 0 | 0 | 0 | 0 | 0 | 0.28 | 0 |
| BM | 0 | 1 | 0 | 1 | 1 | 0 | 0 | 0.28 | 0 |
| BM | 0 | 1 | 0 | 0 | 1 | 0 | 0 | 0.28 | 0 |
| BM | 1 | 1 | 1 | 1 | 1 | 0 | 0 | 0.28 | 0 |
| BM | 0 | 1 | 0 | 1 | 0 | 0 | 0 | 0.28 | 0 |
| BM | 1 | 1 | 0 | 1 | 1 | 0 | 0 | 0.25 | 0 |
| BM | 0 | 0 | 1 | 0 | 0 | 1 | 0 | 0.25 | 0 |
| BM | 0 | 0 | 0 | 0 | 0 | 0 | 0 | 0.25 | 0 |
| PBSC | 0 | 0 | 1 | 0 | 0 | 0 | 0 | 0.25 | 0 |
| BM | 1 | 1 | 0 | 1 | 1 | 0 | 1 | 0.25 | 0 |
| BM | 0 | 0 | 0 | 0 | 0 | 0 | 0 | 0.25 | 0 |
| BM | 0 | 0 | 0 | 0 | 0 | 0 | 0 | 0.22 | 0 |
| BM | 1 | 1 | 1 | 1 | 1 | 0 | 0 | 0.22 | 0 |
| BM | 0 | 1 | 0 | 0 | 1 | 0 | 0 | 0.22 | 0 |
| BM | 0 | 0 | 0 | 0 | 1 | 0 | 0 | 0.22 | 0 |
| BM | 0 | 0 | 0 | 0 | 0 | 0 | 0 | 0.22 | 0 |
| BM | 0 | 0 | 0 | 0 | 0 | 1 | 0 | 0.22 | 0 |
| BM | 0 | 0 | 0 | 0 | 0 | 0 | 0 | 0.22 | 0 |
| BM | 0 | 0 | 0 | 0 | 0 | 0 | 1 | 0.22 | 0 |
| BM | 0 | 0 | 0 | 0 | 1 | 0 | 0 | 0.19 | 0 |
| BM | 0 | 0 | 0 | 0 | 0 | 0 | 0 | 0.19 | 0 |
| BM | 0 | 0 | 0 | 0 | 0 | 0 | 0 | 0.17 | 0 |
| BM | 0 | 0 | 0 | 0 | 0 | 0 | 0 | 0.17 | 0 |
| BM | 0 | 0 | 0 | 0 | 0 | 0 | 0 | 0.17 | 0 |
| BM | 0 | 1 | 0 | 0 | 0 | 0 | 0 | 0.17 | 0 |
| PBSC | 0 | 0 | 0 | 0 | 0 | 0 | 0 | 0.17 | 0 |
| BM | 1 | 0 | 0 | 0 | 0 | 0 | 0 | 0.17 | 0 |
| BM | 0 | 0 | 1 | 0 | 0 | 1 | 0 | 0.17 | 0 |
| BM | 0 | 0 | 0 | 0 | 0 | 0 | 0 | 0.14 | 0 |
| BM | 0 | 0 | 0 | 0 | 0 | 0 | 0 | 0.14 | 0 |
| BM | 1 | 0 | 0 | 0 | 0 | 0 | 1 | 0.14 | 0 |
| BM | 0 | 0 | 0 | 0 | 0 | 0 | 0 | 0.11 | 0 |
| BM | 0 | 0 | 0 | 0 | 0 | 0 | 0 | 0.11 | 0 |
| BM | 0 | 0 | 0 | 0 | 0 | 0 | 0 | 0.11 | 0 |
| BM | 0 | 0 | 0 | 0 | 0 | 0 | 0 | 0.11 | 0 |
| BM | 0 | 0 | 0 | 0 | 0 | 0 | 0 | 0.00 | 0 |

### Example 14

This example includes a description of improved RGP Vmod performance compared to SG (single gene) Vmod performance for GVHD outcome prediction.

Ratiometric gene pairs (RGPs) provide for additional outcome predictive robustness through (1) self-calibration by dividing-out background variation, and (2) capturing potential competitive pathway interaction effects between genes at the expression level. Therefore, when evaluating the performance of a GVHD outcome prediction gene set, one would expect the RGP voting model implementation to provide superior performance compared to a simple SG voting model implementation.

Comparison of SG and RGP Vmod Alternatives for SG43RGP36-RGPgreedysearch:

For example, referring to Table 26, SG43RGP36compSGRGP for Gneg vs. Gag3 outcome predictive performance, the best performing 48 gene GVHD outcome prediction implementation shown above, SG43RGP36-RGPgreedysearch, uses
(1) in simple 43 SG Vmod configuration, 43 predictive genes as individual voters and contributors to the GNOS value, and
(2) in superior 36 RGP Vmod configuration, 43 predictive genes in 36 different pair-wise RGP combinations as individual voters and contributors to the GNOS value (see above for detailed listings, and RGP Vmod performance details).

Note that the 36 RGP Vmod implementation (Table 26, SG43RGP36compSGRGP) outperforms the 43 SG Vmod implementation in every performance category. Beginning with the T-test, 36 RGP p-values are 10 orders of magnitude lower (better) than compared to the 43 SG p-values. GVHD reduction and GVHD N donor capture, at 5 different GNOS threshold levels, is 10%-20% better for the 36 RGP model compared to the 43 SG implementation.

TABLE 26
Comparison of SG43 voting model and RGP36 voting model implementations of Vmod SG43RGP36-RGPgreedysearch,
for the Gneg vs. Gag3 division, at prevalence P = 0.25 (“SG43RGP36compSGRGP”)

| | | | | | | | GVHD N |
|---|---|---|---|---|---|---|---|
| | Gneg vs. | | | TNR Gneg | TPR Gneg | GVHD | donor |
| | Gag3 T-test | GNOS | NPV Gneg | vs. Gag3 | vs. Gag3 | reduction | capture of |
| Vmod | p-value | threshold | vs. Gag3 | (specificity) | (sensitivity) | for Gag3 | Gneg |
| 43 SG Vmod from SG43RGP36-RGPgreedysearch | 2.4E−11 | 0.50 | 0.90 | 0.76 | 0.75 | 61% | 76% |
| 43 SG Vmod from SG43RGP36-RGPgreedysearch | 2.4E−11 | 0.55 | 0.93 | 0.64 | 0.84 | 70% | 64% |
| 43 SG Vmod from SG43RGP36-RGPgreedysearch | 2.4E−11 | 0.65 | 0.96 | 0.37 | 0.95 | 82% | 37% |
| 43 SG Vmod from SG43RGP36-RGPgreedysearch | 2.4E−11 | 0.75 | 1.00 | 0.03 | 1.00 | 100% | 3% |
| 43 SG Vmod from SG43RGP36-RGPgreedysearch | 2.4E−11 | 0.85 | — | 0.00 | 1.00 | — | 0% |
| 36 RGP Vmod from SG43RGP36-RGPgreedysearch | 3.3E−21 | 0.50 | 0.94 | 0.86 | 0.83 | 76% | 86% |
| 36 RGP Vmod from SG43RGP36-RGPgreedysearch | 3.3E−21 | 0.55 | 0.97 | 0.80 | 0.94 | 89% | 80% |
| 36 RGP Vmod from SG43RGP36-RGPgreedysearch | 3.3E−21 | 0.65 | 0.98 | 0.53 | 0.96 | 90% | 53% |
| 36 RGP Vmod from SG43RGP36-RGPgreedysearch | 3.3E−21 | 0.75 | 0.97 | 0.25 | 0.97 | 87% | 25% |
| 36 RGP Vmod from SG43RGP36-RGPgreedysearch | 3.3E−21 | 0.85 | 1.00 | 0.05 | 1.00 | 100% | 5% |

### Example 15

This example includes a description of robust statistical RGP Vmod performance for GVHD outcome prediction when the Vmods were subject to rigorous, state of the art bootstrapped cross-validation.

Bootstrapped cross-validation was applied as a computationally-intensive approach to assess the outcome predictive performance of ratiometric gene pair voting models (RGP Vmods). Bootstrapped cross-validation is more sophisticated technically and more robust in model performance estimation than is conventional cross-validation. (Bradley Efron & Robert J. Tibshirani, An Introduction to the Bootstrap, Chapman & Hall/CRC, Boca Raton, Fla., 1998, esp. pp, 247-255; A. C. Davison & D V Hinkley, Bootstrap Methods and Their Applications, Cambridge University Press, Cambridge, UK, 1997, esp. pp. 292-298.) Bootstrapped cross-validation has inherent advantages over conventional cross-validation, which include: (i) When a bootstrap sampling of the data is drawn for training a model, again and again for nB numbers of independent bootstrap samples on the order of 1000 or more (each independent bootstrap sample comprising the conventional numbers of negative and positive samples in training a model, e.g., 59 Gneg and 121 Gpos for the RRCF data), the resulting empirical distribution of samples used for training much better approximates the underlying distribution of the state-of-nature represented by the data than does any single set of data (this phenomenon is inherent to bootstrap sampling); (ii) also inherent to bootstrap sampling (which is a sampling with replacement), approximately 37% of the data is not selected by any given bootstrap sampling of the data (because by probability theory the fraction of data not selected by a given bootstrap sampling is (1−1/nB)̂nB approx.=0.367, for nB>100; Efron & Tibshirani, pp. 281-282; Davison & Hinkley, p. 114), thereby, inherently providing a corresponding complementary set of data as a test set of samples to be used in the cross-validation phase that was not used in the given bootstrap sample of data used for training the model; and (iii) statistical confidence intervals determined empirically from bootstrap sampling involving nB>1000 are reliable and easy to obtain. In each case analyzed, we applied bootstrapped cross-validation involving nB=10,000 independent bootstrap samplings of the data; hence, a corresponding ensemble of nB=10,000 test sets are generated.

The results of bootstrapped cross-validation on two different RGP Vmods based on RRCF or RL2F RT-PCR data (SG43RGP46-performance and SG43RGP36-RGPgreedysearch) are shown below, each using the GNOS voting threshold=0.5, and for the reasonable and highly likely situation of 30% disease prevalence of grades III or IV acute GVDH (Gag3). Empirically-derived 90-percent confidence intervals around any given performance measure is shown within parentheses.

Vmod SG43RGP46-performance: 0.79 mean sensitivity (0.67,0.90), 0.75 mean specificity (0.58,0.90), 0.76 mean accuracy (0.65,0.86), 0.59 mean positive predictive value (0.45,0.75), and 0.89 mean negative predictive value (0.84,0.94).

Vmod SG43RGP36-RGPgreedysearch: 0.84 mean sensitivity (0.73,0.94), 0.84 mean specificity (0.71,0.95), 0.84 mean accuracy (0.75,0.92), 0.71 mean positive predictive value (0.56,0.88), and 0.93 mean negative predictive value (0.88,0.97).

Thus, these two Vmods are computationally bootstrap cross-validated very successfully to high practical levels of performance, especially in negative predictive value which is particularly important in the medical context of the rate at which, when scored donors are predicted to not induce aGVHD, is a correct prediction of GVHD outcome.

### Example 16

This example includes a description of consistent and robust RGP Vmod performance for GVHD outcome prediction examples with respect to (a) gene expression data measurements originating from different assay platforms, and (b) altered input data modified with noise to reflect potentially confounding measurement and sample behavior variation.

In addition to harnessing the combined ratiometric GVHD outcome predictive and self-calibrating properties of RGPs, further accuracy and robustness in GVHD outcome prediction is expected to be achieved by averaging out errors contributed by individual RGP voters through the use of multi-RGP voting models (Vmods). The combined stabilizing, error-compensating and error-diluting features of multi-gene, multi-RGP voting models would then be expected to provide overall robust outcome prediction, even when:
(1) gene expression is measured using a microarray platform as opposed to the RT-PCR platform, i.e.
   1. using a different method altogether, compared to the measurement method that provided the data on which the training and original performance evaluation was carried out for the RGP Vmods,
   2. using a different method that is commonly accepted to be less accurate and sensitive (microarray gene expression assays are considered potentially noisy and more useful as survey tools, whereas RT-PCR is considered the gold standard for quantitative gene expression analysis, especially with respect to human medical diagnostics), and
(2) gene expression data, originating from different platforms (e.g. microarray and RT-PCR) is distorted and corrupted by various sources of variation due to sample handling, laboratory processing, instrument noise, biological variability, etc. (which can be simulated through the addition of light to extreme levels of computationally generated random noise to existing measurement data).

GVHD outcome predictive performance was determined for 3 different RGP Vmods, based on TaqMan real-time RT-PCR measurements for all 180 samples (RRCF or RL2F data, as described above), and also based on Illumina HT12 v3.0 microarray measurements for exactly the same genes as in RT-PCR assay, for 163 of the 180 samples (VQLS, as described above). Note that the RGP separatrices were determined separately for the RT-PCR and microarray datasets, since the data are on different scales for the RGPs. GNOS values and GNOS thresholds were determined the same way for both datasets.

Robustness of Vmod GVHD Outcome Prediction from RT-PCR and Microarray Measurement Data in the Presence of Noise:

For the RT-PCR and microarray measurement data of the same genes, noise (computationally generated independent random perturbations) was added to the measurement values. Uniform random noise ranging from multiples of +/−0.1× to +/−10× of the SG standard deviation, was added to each SG value for each sample, before calculating the corresponding RGP value. SG standard deviations were specifically determined for each SG over all 180 RT-PCR and 163 microarray measurements. Note that this estimate of the SG standard deviation is designed to err on the high side, because the assessed variation comprises biological variation due to sample class differences, as well as non-specific biological and measurement assay variation. Simulated random noise was sampled from a computational random number generator, and added to the SG measurement values 1,000 different times, and, for each iteration, the RGP and corresponding GNOS values for each Vmod were reported. For each set of GNOS values for each noise sampling, the 5 SSPCs (standard specifications for outcome prediction) were determined. For each of the 7 different levels of noise, the average and standard deviations for the SSPCs were determined for the 3 different divisions, for different GNOS thresholds and GVHD prevalences.

Examples of GVHD outcome predictive performance, based on SG inputs corrupted by 0.1× to 10× standard deviations of noise added to either the RT-PCR or microarray measurement data, are reported below, for 3 GVHD outcome prediction test alternatives, for the Gneg vs. Gag3 division, at GNOS threshold of 0.55 and Gag3 prevalence of 25% (midpoint between the commonly accepted range of 15% to 35%).

Table 27 (SG43RGP36noisecompRRCFVQLS) shows a comparison of how robust GVHD outcome prediction is with respect to corruption by noise, for RT-PCR and microarray data, for the so far best performing 48 gene GVHD outcome prediction implementation, SG43RGP36-RGPgreedysearch. At 0.1×s.d. of noise, the RT-PCR compared to the microarray derived Vmod results show lower (better) log 10 p-values (by ˜4 orders of magnitude) and higher (better) GVHD reduction (by ˜10%), and GVHD reduction and GVHD N donor capture of ˜75% or more. However, at 1.0×s.d. and higher of noise, all of the SSPCs are virtually indistinguishable between the RT-PCR and microarray derived Vmods (with respect to both, average and s.d. of SSPCs). Note that even at 2×s.d. of noise, GVHD reduction and GVHD N donor capture are ˜45% or higher for the RT-PCR and microarray alternatives.

In summary, these results validate the expected robustness inherent to the RGP Vmods, with respect to alternative measurement platforms (e.g., microarray, RT-PCR) and high levels of input data corruption. An at least ˜50% GVHD reduction and GVHD N donor capture should be achievable by the SG43RGP36-RGPgreedysearch GVHD outcome prediction implementation, even under exacerbating circumstances that would lead to such severe input data corruption.

Table 28 (3VmodnoisecompRRCF) shows a comparison for RT-PCR data of how robust GVHD outcome prediction is with respect to corruption by noise for 3 alternative Vmod GVHD outcome prediction implementations, SG43RGP46-RGPperformance, SG43RGP36-RGPgreedysearch, and SG21RGP28-RGPmaxgreedysearch. Note that at 0.1×s.d. of noise, SG21RGP28-RGPmaxgreedysearch shows the best overall performance, followed by SG43RGP36-RGPgreedysearch. However, at 1×s.d. of noise, the SSPCs from SG21RGP28-RGPmaxgreedysearch become virtually indistinguishable from those of SG43RGP46-RGPperformance, while the best performing Vmod is represented by SG43RGP36-RGPgreedysearch. In summary, while SG21RGP28-RGPmaxgreedysearch shows the best performance at low levels of noise, SG43RGP36-RGPgreedysearch is more robust, and the better performer in the presence of corrupting noise, when using RT-PCR data as input to GVHD outcome prediction.

Table 29 (3VmodnoisecompVQLS) shows a comparison for microarray data of how robust GVHD outcome prediction is with respect to corruption by noise for 3 alternative Vmod GVHD outcome prediction implementations, SG43RGP46-RGPperformance, SG43RGP36-RGPgreedysearch, and SG21RGP28-RGPmaxgreedysearch. Note that at all levels of noise, the SSPCs from SG21RGP28-RGPmaxgreedysearch are virtually indistinguishable from those of SG43RGP46-RGPperformance, while the best performing Vmod is represented by SG43RGP36-RGPgreedysearch. In summary, SG43RGP36-RGPgreedysearch is the most robust and best performer in the presence of corrupting noise, compared to SG21RGP28-RGPmaxgreedysearch and SG43RGP46-RGPperformance, when using microarray data as input for GVHD outcome prediction.

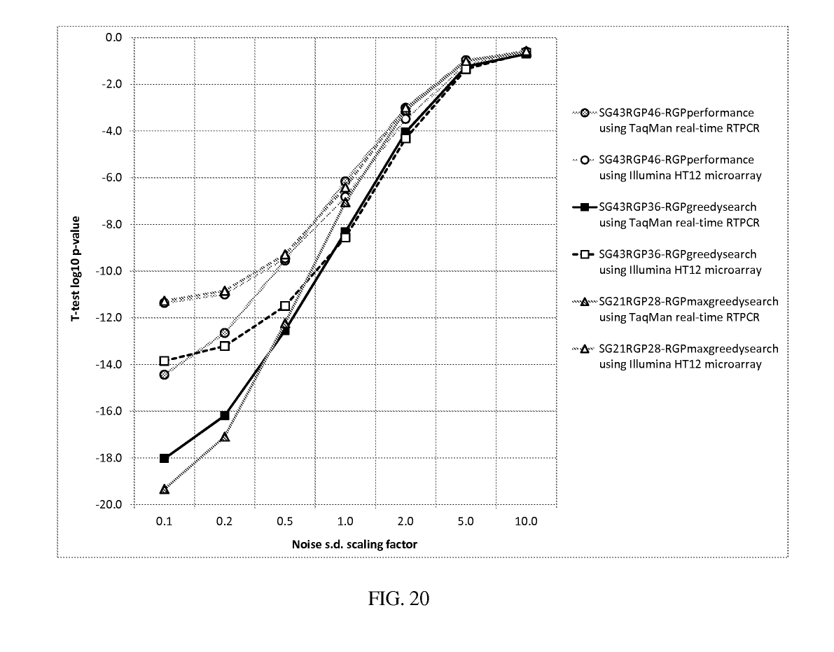

FIG. 20 (3VmodnoisecompTtest) shows a comparison of how robust GVHD outcome predictive p-values are with respect to corruption by noise, for RT-PCR and microarray data, for the 3 alternative Vmod GVHD outcome prediction implementations, SG43RGP46-RGPperformance, SG43RGP36-RGPgreedysearch, and SG21RGP28-RGPmaxgreedysearch. The lowest p-values, as observed for SG43RGP36-RGPgreedysearch and SG21RGP28-RGPmaxgreedysearch, are only observed when RT-PCR and not microarray measurements are used. Also, the p-values for all of the models, whether using RT-PCR or microarray data, are essentially robust to perturbations with up to 0.5×s.d. of noise added, and still in the very low ˜10⁻⁶ to ˜10⁻⁸ range at 1×s.d. of noise added. However, p-values become noticeably corrupted at 2×s.d. of noise added, though still in a potentially useful range for a GVHD outcome prediction test. At >=5×s.d. of noise added (which is very large amount of noise), the outcome predictive p-values are essentially completely corrupted. Also, at 1×s.d. of noise added, the Vmod SG43RGP36-RGPgreedysearch shows the lowest p-values, i.e. <10⁻⁸, compared to all other Vmods, whether using RT-PCR or microarray data, and demonstrates better robustness to corruption by noise compared to the more “highly tuned” SG21RGP28-RGPmaxgreedysearch.

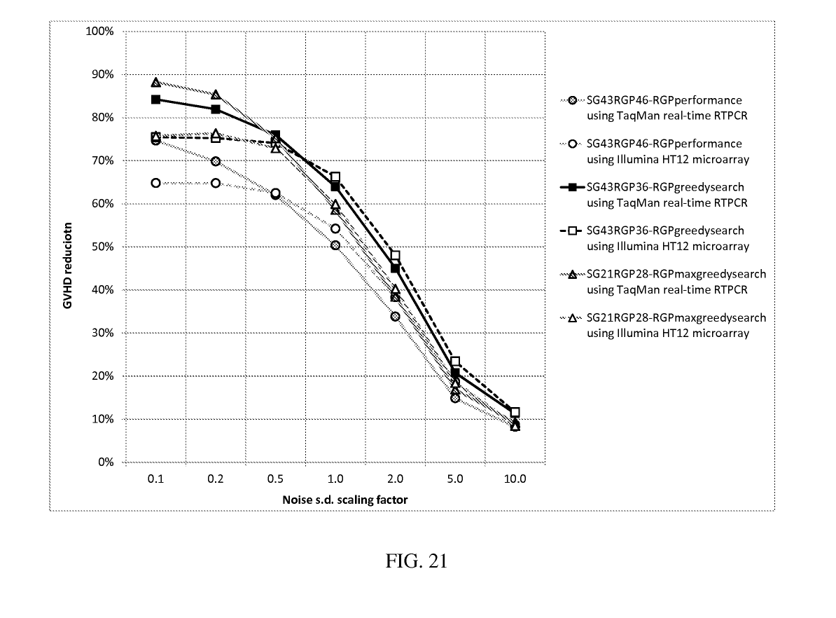

FIG. 21 (3VmodnoisecompGVHDred) shows a comparison of how robust projected GVHD reduction is with respect to corruption by noise, for RT-PCR and microarray data, for the 3 alternative Vmod GVHD outcome prediction implementations, SG43RGP46-RGPperformance, SG43RGP36-RGPgreedysearch, and SG21RGP28-RGPmaxgreedysearch. The highest projected GVHD reduction, in the 80% to 90% range, observed for SG43RGP36-RGPgreedysearch and SG21RGP28-RGPmaxgreedysearch, is only seen when RT-PCR and not microarray measurements are used, and is robust to corruption by 0.1× to 0.2×s.d. noise added. At 0.5× to 1×s.d. of noise added, all Vmods consistently show GVHD reduction in the 50% to 75% range. Even at 2×s.d. of noise added, GVHD reduction is still projected in the 35% to 50% range. At >=5×s.d. of noise added (which is a substantial amount of noise added), the projected GVHD reductions are virtually completely corrupted.

Interestingly, projected GHVD reduction at more than 0.5×s.d. of noise added for all Vmods is higher when using microarray compared to RT-PCR data, even though RT-PCR data was used for selecting the RGPs and designing the Vmods. Also, at 1×s.d. of noise added, of all the Vmods, SG43RGP36-RGPgreedysearch shows the highest, i.e. ˜65% projected GVHD reduction for the RT-PCR as well as microarray data versions.

Conclusion and Prioritization for Clinical Implementation:

Overall, SG43RGP36-RGPgreedysearch performs most robustly, with the best SSPCs, for RT-PCR as well as microarray data, in the presence of medium levels of noise (up to 1×s.d. of noise), compared to SG21RGP28-RGPmaxgreedysearch and SG43RGP46-RGPperformance. However, at low levels of noise when using RT-PCR data, SG21RGP28-RGPmaxgreedysearch shows the best SSPCs. Moreover, the differences between the 3 GVHD outcome prediction Vmod alternatives are more pronounced when using RT-PCR compared to microarray data. This may be due to the expected higher fidelity and accuracy of the RT-PCR data compared to microarray data. Thus, GVHD outcome prediction implementations using the microarray and RT-PCR data are both plausible, but RT-PCR offers the highest fidelity for overall superior GVHD outcome prediction performance.

For practical clinical applications of GVHD outcome prediction, there may be advantages to using SG43RGP36-RGPgreedysearch in terms of combined excellent GVHD N outcome predictive performance and robustness. Because all the SGs and RGPs of the (potentially superior, at low noise) SG21RGP28-RGPmaxgreedysearch Vmod are also contained in SG43RGP36-RGPgreedysearch, the outputs for SG21RGP28-RGPmaxgreedysearch can be determined from the same measurements as SG43RGP36-RGPgreedysearch. Therefore, while GVHD N outcome prediction using SG43RGP36-RGPgreedysearch would currently be considered most reliable, parallel investigational evaluation of SG21RGP28-RGPmaxgreedysearch will determine the benefits and application of this Vmod from the processing of the pertinent subset of the same measurement data used for GVHD outcome prediction with Vmod SG43RGP36-RGPgreedysearch.

TABLE 27
Comparison of SG43RGP36-RGPgreedysearch performance for RT-PCR and microarray gene expression, in the presence of noise,
ranging from 0.1x to 10x of SG standard deviation, for the Gneg vs. Gag3 division (“SG43RGP36noisecompRRCFVQLS”),
at a GNOS threshold of 0.55 and prevalence P = 0.25 (average and s.d. of performance values over 1,000 iterations of noise)

| | | | | | AVG | AVG | AVG | AVG | AVG |
|---|---|---|---|---|---|---|---|---|---|
| | | | Preva- | Noise | NPV | TNR | TPR | ACC | GVHD |
| | | GNOS | lence | s.d. | Gneg | (specificity) | (sensitivity) | (accuracy) | reduc- |
| | Gene expression | thresh- | for | scaling | vs. | Gneg vs. | Gneg vs. | Gneg vs. | tion |
| Vmod | measurement platform | old | Gag3 | factor | Gag3 | Gag3 | Gag3 | Gag3 | for Gag3 |
| SG43RGP36-RGPgreedysearch | TaqMan real-time RT-PCR | 0.55 | 25% | 0.1 | 0.96 | 0.74 | 0.91 | 0.78 | 84% |
| SG43RGP36-RGPgreedysearch | TaqMan real-time RT-PCR | 0.55 | 25% | 0.2 | 0.95 | 0.71 | 0.90 | 0.76 | 82% |
| SG43RGP36-RGPgreedysearch | TaqMan real-time RT-PCR | 0.55 | 25% | 0.5 | 0.94 | 0.66 | 0.87 | 0.71 | 76% |
| SG43RGP36-RGPgreedysearch | TaqMan real-time RT-PCR | 0.55 | 25% | 1.0 | 0.91 | 0.59 | 0.83 | 0.65 | 64% |
| SG43RGP36-RGPgreedysearch | TaqMan real-time RT-PCR | 0.55 | 25% | 2.0 | 0.86 | 0.50 | 0.76 | 0.57 | 45% |
| SG43RGP36-RGPgreedysearch | TaqMan real-time RT-PCR | 0.55 | 25% | 5.0 | 0.80 | 0.42 | 0.69 | 0.49 | 21% |
| SG43RGP36-RGPgreedysearch | TaqMan real-time RT-PCR | 0.55 | 25% | 10.0 | 0.78 | 0.40 | 0.66 | 0.47 | 11% |
| SG43RGP36-RGPgreedysearch | Illumina HT12 microarray | 0.55 | 25% | 0.1 | 0.94 | 0.75 | 0.85 | 0.77 | 75% |
| SG43RGP36-RGPgreedysearch | Illumina HT12 microarray | 0.55 | 25% | 0.2 | 0.94 | 0.73 | 0.86 | 0.76 | 75% |
| SG43RGP36-RGPgreedysearch | Illumina HT12 microarray | 0.55 | 25% | 0.5 | 0.94 | 0.68 | 0.86 | 0.73 | 74% |
| SG43RGP36-RGPgreedysearch | Illumina HT12 microarray | 0.55 | 25% | 1.0 | 0.92 | 0.62 | 0.83 | 0.67 | 66% |
| SG43RGP36-RGPgreedysearch | Illumina HT12 microarray | 0.55 | 25% | 2.0 | 0.87 | 0.52 | 0.77 | 0.58 | 48% |
| SG43RGP36-RGPgreedysearch | Illumina HT12 microarray | 0.55 | 25% | 5.0 | 0.81 | 0.43 | 0.70 | 0.50 | 23% |
| SG43RGP36-RGPgreedysearch | Illumina HT12 microarray | 0.55 | 25% | 10.0 | 0.78 | 0.40 | 0.67 | 0.47 | 12% |

| | AVG | AVG | | | | | | | SDV |
|---|---|---|---|---|---|---|---|---|---|
| | GVHD N | Gneg vs. | | SDV | SDV | | | SDV | Gneg vs. |
| | donor | Gag3 T-test | | TNR | TPR | SDV | SDV | GVHD N | Gag3 T-test |
| | capture of | log10 | SDV | (spec- | (sensi- | ACC | GVHD | donor | log10 |
| Vmod | Gneg | p-value | NPV | ificity) | tivity) | (accuracy) | reduction | capture | p-value |
| SG43RGP36-RGPgreedysearch | 74% | −18.03 | 0.01 | 0.03 | 0.02 | 0.02 | 3% | 3% | 0.69 |
| SG43RGP36-RGPgreedysearch | 71% | −16.19 | 0.01 | 0.03 | 0.02 | 0.02 | 4% | 3% | 1.00 |
| SG43RGP36-RGPgreedysearch | 66% | −12.55 | 0.01 | 0.04 | 0.03 | 0.03 | 5% | 4% | 1.47 |
| SG43RGP36-RGPgreedysearch | 59% | −8.32 | 0.02 | 0.05 | 0.04 | 0.04 | 8% | 5% | 1.76 |
| SG43RGP36-RGPgreedysearch | 50% | −4.05 | 0.03 | 0.06 | 0.05 | 0.05 | 11% | 6% | 1.52 |
| SG43RGP36-RGPgreedysearch | 42% | −1.24 | 0.04 | 0.06 | 0.05 | 0.05 | 15% | 6% | 0.96 |
| SG43RGP36-RGPgreedysearch | 40% | −0.70 | 0.04 | 0.06 | 0.05 | 0.05 | 16% | 6% | 0.67 |
| SG43RGP36-RGPgreedysearch | 75% | −13.85 | 0.01 | 0.02 | 0.02 | 0.02 | 3% | 2% | 0.60 |
| SG43RGP36-RGPgreedysearch | 73% | −13.21 | 0.01 | 0.03 | 0.03 | 0.02 | 4% | 3% | 0.81 |
| SG43RGP36-RGPgreedysearch | 68% | −11.50 | 0.01 | 0.04 | 0.03 | 0.03 | 6% | 4% | 1.31 |
| SG43RGP36-RGPgreedysearch | 62% | −8.56 | 0.02 | 0.06 | 0.04 | 0.04 | 8% | 6% | 1.69 |
| SG43RGP36-RGPgreedysearch | 52% | −4.32 | 0.03 | 0.06 | 0.05 | 0.05 | 11% | 6% | 1.50 |
| SG43RGP36-RGPgreedysearch | 43% | −1.35 | 0.04 | 0.06 | 0.05 | 0.05 | 15% | 6% | 0.93 |
| SG43RGP36-RGPgreedysearch | 40% | −0.65 | 0.04 | 0.07 | 0.06 | 0.05 | 17% | 7% | 0.64 |

TABLE 28
Comparison of Vmod performance in the presence of noise, ranging from 0.1x to 10x of SG
measurement standard deviation, for the Gneg vs. Gag3 division, (“3VmodnoisecompRRCF”) at a GNOS
threshold of 0.55 and prevalence P = 0.25 (average and s.d. of performance values over 1,000 iterations of noise)

| | | | | | | AVG | AVG | AVG | AVG | AVG |
|---|---|---|---|---|---|---|---|---|---|---|
| | Gene | | | Noise | AVG | TNR | TPR | ACC | GVHD | GVHD |
| | expression | | | s.d. | NPV | (specificity) | (sensitivity) | (accuracy) | reduction | N donor |
| | measurement | GNOS | Prevalence | scaling | Gneg vs. | Gneg vs. | Gneg vs. | Gneg vs. | for | capture |
| Vmod | platform | threshold | for Gag3 | factor | Gag3 | Gag3 | Gag3 | Gag3 | Gag3 | of Gneg |
| SG43RGP46- | TaqMan real-time | 0.55 | 25% | 0.1 | 0.94 | 0.73 | 0.85 | 0.76 | 75% | 73% |
| RGPperformance | RT-PCR | | | | | | | | | |
| SG43RGP46- | TaqMan real-time | 0.55 | 25% | 0.2 | 0.92 | 0.70 | 0.83 | 0.74 | 70% | 70% |
| RGPperformance | RT-PCR | | | | | | | | | |
| SG43RGP46- | TaqMan real-time | 0.55 | 25% | 0.5 | 0.91 | 0.67 | 0.79 | 0.70 | 62% | 67% |
| RGPperformance | RT-PCR | | | | | | | | | |
| SG43RGP46- | TaqMan real-time | 0.55 | 25% | 1.0 | 0.88 | 0.61 | 0.74 | 0.64 | 50% | 61% |
| RGPperformance | RT-PCR | | | | | | | | | |
| SG43RGP46- | TaqMan real-time | 0.55 | 25% | 2.0 | 0.83 | 0.55 | 0.68 | 0.58 | 34% | 55% |
| RGPperformance | RT-PCR | | | | | | | | | |
| SG43RGP46- | TaqMan real-time | 0.55 | 25% | 5.0 | 0.79 | 0.49 | 0.61 | 0.52 | 15% | 49% |
| RGPperformance | RT-PCR | | | | | | | | | |
| SG43RGP46- | TaqMan real-time | 0.55 | 25% | 10.0 | 0.77 | 0.47 | 0.59 | 0.50 | 8% | 47% |
| RGPperformance | RT-PCR | | | | | | | | | |
| SG43RGP36- | TaqMan real-time | 0.55 | 25% | 0.1 | 0.96 | 0.74 | 0.91 | 0.78 | 84% | 74% |
| RGPgreedysearch | RT-PCR | | | | | | | | | |
| SG43RGP36- | TaqMan real-time | 0.55 | 25% | 0.2 | 0.95 | 0.71 | 0.90 | 0.76 | 82% | 71% |
| RGPgreedysearch | RT-PCR | | | | | | | | | |
| SG43RGP36- | TaqMan real-time | 0.55 | 25% | 0.5 | 0.94 | 0.66 | 0.87 | 0.71 | 76% | 66% |
| RGPgreedysearch | RT-PCR | | | | | | | | | |
| SG43RGP36- | TaqMan real-time | 0.55 | 25% | 1.0 | 0.91 | 0.59 | 0.83 | 0.65 | 64% | 59% |
| RGPgreedysearch | RT-PCR | | | | | | | | | |
| SG43RGP36- | TaqMan real-time | 0.55 | 25% | 2.0 | 0.86 | 0.50 | 0.76 | 0.57 | 45% | 50% |
| RGPgreedysearch | RT-PCR | | | | | | | | | |
| SG43RGP36- | TaqMan real-time | 0.55 | 25% | 5.0 | 0.80 | 0.42 | 0.69 | 0.49 | 21% | 42% |
| RGPgreedysearch | RT-PCR | | | | | | | | | |
| SG43RGP36- | TaqMan real-time | 0.55 | 25% | 10.0 | 0.78 | 0.40 | 0.66 | 0.47 | 11% | 40% |
| RGPgreedysearch | RT-PCR | | | | | | | | | |
| SG21RGP28- | TaqMan real-time | 0.55 | 25% | 0.1 | 0.97 | 0.75 | 0.93 | 0.79 | 88% | 75% |
| RGPmaxgreedysearch | RT-PCR | | | | | | | | | |
| SG21RGP28- | TaqMan real-time | 0.55 | 25% | 0.2 | 0.96 | 0.71 | 0.92 | 0.76 | 85% | 71% |
| RGPmaxgreedysearch | RT-PCR | | | | | | | | | |
| SG21RGP28- | TaqMan real-time | 0.55 | 25% | 0.5 | 0.94 | 0.63 | 0.87 | 0.69 | 75% | 63% |
| RGPmaxgreedysearch | RT-PCR | | | | | | | | | |
| SG21RGP28- | TaqMan real-time | 0.55 | 25% | 1.0 | 0.90 | 0.56 | 0.81 | 0.62 | 59% | 56% |
| RGPmaxgreedysearch | RT-PCR | | | | | | | | | |
| SG21RGP28- | TaqMan real-time | 0.55 | 25% | 2.0 | 0.85 | 0.48 | 0.74 | 0.55 | 38% | 48% |
| RGPmaxgreedysearch | RT-PCR | | | | | | | | | |
| SG21RGP28- | TaqMan real-time | 0.55 | 25% | 5.0 | 0.79 | 0.42 | 0.67 | 0.48 | 17% | 42% |
| RGPmaxgreedysearch | RT-PCR | | | | | | | | | |
| SG21RGP28- | TaqMan real-time | 0.55 | 25% | 10.0 | 0.77 | 0.40 | 0.65 | 0.46 | 9% | 40% |
| RGPmaxgreedysearch | RT-PCR | | | | | | | | | |

| | | AVG | | | | | | | SDV |
|---|---|---|---|---|---|---|---|---|---|
| | Gene | Gneg vs. | | | | | | SDV | Gneg vs. |
| | expression | Gag3 | | SDV | SDV | SDV | SDV | GVHD | Gag3 |
| | measurement | T-test log10 | SDV | TNR | TPR | ACC | GVHD | N donor | T-test log10 |
| Vmod | platform | p-value | NPV | (specificity) | (sensitivity) | (accuracy) | reduction | capture | p-value |
| SG43RGP46- | TaqMan real-time | −14.44 | 0.01 | 0.03 | 0.02 | 0.02 | 3% | 3% | 0.72 |
| RGPperformance | RT-PCR | | | | | | | | |
| SG43RGP46- | TaqMan real-time | −12.65 | 0.01 | 0.03 | 0.02 | 0.02 | 4% | 3% | 0.98 |
| RGPperformance | RT-PCR | | | | | | | | |
| SG43RGP46- | TaqMan real-time | −9.55 | 0.01 | 0.04 | 0.03 | 0.03 | 5% | 4% | 1.39 |
| RGPperformance | RT-PCR | | | | | | | | |
| SG43RGP46- | TaqMan real-time | -6.16 | 0.02 | 0.05 | 0.04 | 0.04 | 8% | 5% | 1.58 |
| RGPperformance | RT-PCR | | | | | | | | |
| SG43RGP46- | TaqMan real-time | −3.01 | 0.03 | 0.06 | 0.05 | 0.05 | 11% | 6% | 1.37 |
| RGPperformance | RT-PCR | | | | | | | | |
| SG43RGP46- | TaqMan real-time | −0.97 | 0.03 | 0.06 | 0.06 | 0.05 | 13% | 6% | 0.80 |
| RGPperformance | RT-PCR | | | | | | | | |
| SG43RGP46- | TaqMan real-time | −0.61 | 0.04 | 0.06 | 0.06 | 0.05 | 15% | 6% | 0.62 |
| RGPperformance | RT-PCR | | | | | | | | |
| SG43RGP36- | TaqMan real-time | −18.03 | 0.01 | 0.03 | 0.02 | 0.02 | 3% | 3% | 0.69 |
| RGPgreedysearch | RT-PCR | | | | | | | | |
| SG43RGP36- | TaqMan real-time | −16.19 | 0.01 | 0.03 | 0.02 | 0.02 | 4% | 3% | 1.00 |
| RGPgreedysearch | RT-PCR | | | | | | | | |
| SG43RGP36- | TaqMan real-time | −12.55 | 0.01 | 0.04 | 0.03 | 0.03 | 5% | 4% | 1.47 |
| RGPgreedysearch | RT-PCR | | | | | | | | |
| SG43RGP36- | TaqMan real-time | −8.32 | 0.02 | 0.05 | 0.04 | 0.04 | 8% | 5% | 1.76 |
| RGPgreedysearch | RT-PCR | | | | | | | | |
| SG43RGP36- | TaqMan real-time | −4.05 | 0.03 | 0.06 | 0.05 | 0.05 | 11% | 6% | 1.52 |
| RGPgreedysearch | RT-PCR | | | | | | | | |
| SG43RGP36- | TaqMan real-time | −1.24 | 0.04 | 0.06 | 0.05 | 0.05 | 15% | 6% | 0.96 |
| RGPgreedysearch | RT-PCR | | | | | | | | |
| SG43RGP36- | TaqMan real-time | −0.70 | 0.04 | 0.06 | 0.05 | 0.05 | 16% | 6% | 0.67 |
| RGPgreedysearch | RT-PCR | | | | | | | | |
| SG21RGP28- | TaqMan real-time | −19.33 | 0.01 | 0.03 | 0.01 | 0.02 | 2% | 3% | 0.93 |
| RGPmaxgreedysearch | RT-PCR | | | | | | | | |
| SG21RGP28- | TaqMan real-time | −17.09 | 0.01 | 0.03 | 0.02 | 0.03 | 3% | 3% | 1.29 |
| RGPmaxgreedysearch | RT-PCR | | | | | | | | |
| SG21RGP28- | TaqMan real-time | −12.23 | 0.01 | 0.05 | 0.03 | 0.04 | 6% | 5% | 1.77 |
| RGPmaxgreedysearch | RT-PCR | | | | | | | | |
| SG21RGP28- | TaqMan real-time | −7.05 | 0.02 | 0.06 | 0.04 | 0.04 | 9% | 6% | 1.80 |
| RGPmaxgreedysearch | RT-PCR | | | | | | | | |
| SG21RGP28- | TaqMan real-time | −3.11 | 0.03 | 0.06 | 0.05 | 0.05 | 12% | 6% | 1.39 |
| RGPmaxgreedysearch | RT-PCR | | | | | | | | |
| SG21RGP28- | TaqMan real-time | −1.00 | 0.04 | 0.06 | 0.05 | 0.05 | 15% | 6% | 0.83 |
| RGPmaxgreedysearch | RT-PCR | | | | | | | | |
| SG21RGP28- | TaqMan real-time | −0.63 | 0.04 | 0.06 | 0.06 | 0.05 | 16% | 6% | 0.60 |
| RGPmaxgreedysearch | RT-PCR | | | | | | | | |

TABLE 29
Comparison of Vmod performance in the presence of noise, ranging from 0.1x to 10x of SG
measurement standard deviation, for the Gneg vs. Gag3 division (“3VmodnoisecompVQLS”), at a GNOS
threshold of 0.55 and prevalence P = 0.25 (average and s.d. of performance values over 1,000 iterations of noise)

| | | | | | | AVG | AVG | AVG | AVG | AVG |
|---|---|---|---|---|---|---|---|---|---|---|
| | Gene | | | | AVG | TNR | TPR | ACC | GVHD | GVHD |
| | expression | | | Noise s.d. | NPV | (specificity) | (sensitivity) | (accuracy) | reduction | N donor |
| | measurement | GNOS | Prevalence | scaling | Gneg vs. | Gneg vs. | Gneg vs. | Gneg vs. | for | capture |
| Vmod | platform | threshold | for Gag3 | factor | Gag3 | Gag3 | Gag3 | Gag3 | Gag3 | of Gneg |
| SG43RGP46- | Illumina HT12 | 0.55 | 25% | 0.1 | 0.91 | 0.75 | 0.78 | 0.76 | 65% | 75% |
| RGPperformance | microarray | | | | | | | | | |
| SG43RGP46- | Illumina HT12 | 0.55 | 25% | 0.2 | 0.91 | 0.75 | 0.78 | 0.76 | 65% | 75% |
| RGPperformance | microarray | | | | | | | | | |
| SG43RGP46- | Illumina HT12 | 0.55 | 25% | 0.5 | 0.91 | 0.72 | 0.78 | 0.73 | 62% | 72% |
| RGPperformance | microarray | | | | | | | | | |
| SG43RGP46- | Illumina HT12 | 0.55 | 25% | 1.0 | 0.89 | 0.65 | 0.75 | 0.68 | 54% | 65% |
| RGPperformance | microarray | | | | | | | | | |
| SG43RGP46- | Illumina HT12 | 0.55 | 25% | 2.0 | 0.85 | 0.58 | 0.69 | 0.60 | 38% | 58% |
| RGPperformance | microarray | | | | | | | | | |
| SG43RGP46- | Illumina HT12 | 0.55 | 25% | 5.0 | 0.80 | 0.49 | 0.63 | 0.53 | 19% | 49% |
| RGPperformance | microarray | | | | | | | | | |
| SG43RGP46- | Illumina HT12 | 0.55 | 25% | 10.0 | 0.77 | 0.46 | 0.59 | 0.50 | 9% | 46% |
| RGPperformance | microarray | | | | | | | | | |
| SG43RGP36- | Illumina HT12 | 0.55 | 25% | 0.1 | 0.94 | 0.75 | 0.85 | 0.77 | 75% | 75% |
| RGPgreedysearch | microarray | | | | | | | | | |
| SG43RGP36- | Illumina HT12 | 0.55 | 25% | 0.2 | 0.94 | 0.73 | 0.86 | 0.76 | 75% | 73% |
| RGPgreedysearch | microarray | | | | | | | | | |
| SG43RGP36- | Illumina HT12 | 0.55 | 25% | 0.5 | 0.94 | 0.68 | 0.86 | 0.73 | 74% | 68% |
| RGPgreedysearch | microarray | | | | | | | | | |
| SG43RGP36- | Illumina HT12 | 0.55 | 25% | 1.0 | 0.92 | 0.62 | 0.83 | 0.67 | 66% | 62% |
| RGPgreedysearch | microarray | | | | | | | | | |
| SG43RGP36- | Illumina HT12 | 0.55 | 25% | 2.0 | 0.87 | 0.52 | 0.77 | 0.58 | 48% | 52% |
| RGPgreedysearch | microarray | | | | | | | | | |
| SG43RGP36- | Illumina HT12 | 0.55 | 25% | 5.0 | 0.81 | 0.43 | 0.70 | 0.50 | 23% | 43% |
| RGPgreedysearch | microarray | | | | | | | | | |
| SG43RGP36- | Illumina HT12 | 0.55 | 25% | 10.0 | 0.78 | 0.40 | 0.67 | 0.47 | 12% | 40% |
| RGPgreedysearch | microarray | | | | | | | | | |
| SG21RGP28- | Illumina HT12 | 0.55 | 25% | 0.1 | 0.94 | 0.68 | 0.87 | 0.73 | 76% | 68% |
| RGPmaxgreedysearch | microarray | | | | | | | | | |
| SG21RGP28- | Illumina HT12 | 0.55 | 25% | 0.2 | 0.94 | 0.68 | 0.87 | 0.73 | 76% | 68% |
| RGPmaxgreedysearch | microarray | | | | | | | | | |
| SG21RGP28- | Illumina HT12 | 0.55 | 25% | 0.5 | 0.93 | 0.63 | 0.86 | 0.69 | 73% | 63% |
| RGPmaxgreedysearch | microarray | | | | | | | | | |
| SG21RGP28- | Illumina HT12 | 0.55 | 25% | 1.0 | 0.90 | 0.57 | 0.81 | 0.63 | 60% | 57% |
| RGPmaxgreedysearch | microarray | | | | | | | | | |
| SG21RGP28- | Illumina HT12 | 0.55 | 25% | 2.0 | 0.85 | 0.49 | 0.74 | 0.56 | 40% | 49% |
| RGPmaxgreedysearch | microarray | | | | | | | | | |
| SG21RGP28- | Illumina HT12 | 0.55 | 25% | 5.0 | 0.80 | 0.42 | 0.68 | 0.49 | 18% | 42% |
| RGPmaxgreedysearch | microarray | | | | | | | | | |
| SG21RGP28- | Illumina HT12 | 0.55 | 25% | 10.0 | 0.77 | 0.40 | 0.65 | 0.46 | 8% | 40% |
| RGPmaxgreedysearch | microarray | | | | | | | | | |

| | | AVG | | | | | | | SDV |
|---|---|---|---|---|---|---|---|---|---|
| | Gene | Gneg vs. | | | | | | SDV | Gneg vs. |
| | expression | Gag3 | | SDV | SDV | SDV | SDV | GVHD | Gag3 |
| | measurement | T-test log10 | SDV | TNR | TPR | ACC | GVHD | N donor | T-test log10 |
| Vmod | platform | p-value | NPV | (specificity) | (sensitivity) | (accuracy) | reduction | capture | p-value |
| SG43RGP46- | Illumina HT12 | −11.36 | 0.01 | 0.02 | 0.02 | 0.02 | 3% | 2% | 0.57 |
| RGPperformance | microarray | | | | | | | | |
| SG43RGP46- | Illumina HT12 | −11.00 | 0.01 | 0.03 | 0.03 | 0.02 | 4% | 3% | 0.79 |
| RGPperformance | microarray | | | | | | | | |
| SG43RGP46- | Illumina HT12 | −9.45 | 0.01 | 0.04 | 0.03 | 0.03 | 6% | 4% | 1.26 |
| RGPperformance | microarray | | | | | | | | |
| SG43RGP46- | Illumina HT12 | −6.82 | 0.02 | 0.05 | 0.04 | 0.04 | 8% | 5% | 1.58 |
| RGPperformance | microarray | | | | | | | | |
| SG43RGP46- | Illumina HT12 | −3.49 | 0.03 | 0.06 | 0.05 | 0.05 | 10% | 6% | 1.34 |
| RGPperformance | microarray | | | | | | | | |
| SG43RGP46- | Illumina HT12 | −1.17 | 0.03 | 0.07 | 0.06 | 0.05 | 14% | 7% | 0.87 |
| RGPperformance | microarray | | | | | | | | |
| SG43RGP46- | Illumina HT12 | −0.60 | 0.04 | 0.07 | 0.06 | 0.05 | 15% | 7% | 0.61 |
| RGPperformance | microarray | | | | | | | | |
| SG43RGP36- | Illumina HT12 | −13.85 | 0.01 | 0.02 | 0.02 | 0.02 | 3% | 2% | 0.60 |
| RGPgreedysearch | microarray | | | | | | | | |
| SG43RGP36- | Illumina HT12 | −13.21 | 0.01 | 0.03 | 0.03 | 0.02 | 4% | 3% | 0.81 |
| RGPgreedysearch | microarray | | | | | | | | |
| SG43RGP36- | Illumina HT12 | −11.50 | 0.01 | 0.04 | 0.03 | 0.03 | 6% | 4% | 1.31 |
| RGPgreedysearch | microarray | | | | | | | | |
| SG43RGP36- | Illumina HT12 | −8.56 | 0.02 | 0.06 | 0.04 | 0.04 | 8% | 6% | 1.69 |
| RGPgreedysearch | microarray | | | | | | | | |
| SG43RGP36- | Illumina HT12 | −4.32 | 0.03 | 0.06 | 0.05 | 0.05 | 11% | 6% | 1.50 |
| RGPgreedysearch | microarray | | | | | | | | |
| SG43RGP36- | Illumina HT12 | −1.35 | 0.04 | 0.06 | 0.05 | 0.05 | 15% | 6% | 0.93 |
| RGPgreedysearch | microarray | | | | | | | | |
| SG43RGP36- | Illumina HT12 | −0.65 | 0.04 | 0.07 | 0.06 | 0.05 | 17% | 7% | 0.64 |
| RGPgreedysearch | microarray | | | | | | | | |
| SG21RGP28- | Illumina HT12 | −11.27 | 0.01 | 0.03 | 0.02 | 0.02 | 4% | 3% | 0.63 |
| RGPmaxgreedysearch | microarray | | | | | | | | |
| SG21RGP28- | Illumina HT12 | −10.84 | 0.01 | 0.04 | 0.03 | 0.03 | 5% | 4% | 0.86 |
| RGPmaxgreedysearch | microarray | | | | | | | | |
| SG21RGP28- | Illumina HT12 | −9.29 | 0.02 | 0.05 | 0.03 | 0.04 | 6% | 5% | 1.36 |
| RGPmaxgreedysearch | microarray | | | | | | | | |
| SG21RGP28- | Illumina HT12 | −6.41 | 0.02 | 0.06 | 0.04 | 0.04 | 9% | 6% | 1.64 |
| RGPmaxgreedysearch | microarray | | | | | | | | |
| SG21RGP28- | Illumina HT12 | −3.00 | 0.03 | 0.06 | 0.05 | 0.05 | 12% | 6% | 1.27 |
| RGPmaxgreedysearch | microarray | | | | | | | | |
| SG21RGP28- | Illumina HT12 | −1.00 | 0.04 | 0.07 | 0.06 | 0.05 | 16% | 7% | 0.77 |
| RGPmaxgreedysearch | microarray | | | | | | | | |
| SG21RGP28- | Illumina HT12 | −0.56 | 0.04 | 0.07 | 0.06 | 0.05 | 17% | 7% | 0.57 |
| RGPmaxgreedysearch | microarray | | | | | | | | |

### Example 17

This example includes a discussion of additional evidence related to the biological basis of GVHD outcome prediction, and GVHD outcome predictive analysis, by comparison of absolute to relative RT-PCR gene expression data.

Absolute gene expression is assessed directly from the output of the RT-PCR measurement instrumentation, as described above for RL2F (including outlier and non-detectable value replacement; see above, “Implemented RT-PCR data pre-processing in 4 steps to arrive at RRCF values,” on which GVHD outcome prediction determinations are based). As such, absolute gene expression assays are subject to many sources of fluctuations (variations in starting material, sample handling and processing, cell metabolic state, instrumentation calibration) that can be compensated for by relative quantitation procedures, such as carried out for RRCF and RGPs (see above: “Implemented RT-PCR data pre-processing in 4 steps to arrive at RRCF values,” on which GVHD outcome prediction determinations are based; and, “Determination of RGPs”).

Consequently, absolute gene expression is generally not used for human diagnostic applications. However, given statistical/numerical safeguards and additional QC checkpoints, absolute gene expression could be applied to dependable human diagnostic applications.

Note: For application to the RGPs that are used in the GVHD outcome prediction test, it is inconsequential whether relative RRCF or absolute RL2F data are used as input to the GVHD outcome prediction test (see above, “Determination of RGPs”)

GVHD Outcome Prediction from RL2F Data:

When evaluating GVHD outcome prediction based on RL2F data (absolute RT-PCR quantition), it is observed that there are ˜2 times as many of the 175 selected genes with p-values<=0.05 (see Table 30, RL2FRRCFSGcomp). The geometric mean of the T-test p-values for Gneg vs. Gag2 is 0.0458, much lower compared to the corresponding RRCF values. (Note: geometric mean is the traditional recommended method for averaging statistical p-values. E.g., the geometric mean of p1=0.00001 and p2=0.1 is p=0.001; whereas, the arithmetic mean would be a misleading p=0.05.)

Remarkably, 95% or the RL2F genes (from the set of 175, see Table 13, SG175) are P-directional, meaning that the average gene expression levels of Gpos, Gag2 or Gag3 samples are higher than in Gneg samples. In comparison, P-directional genes only represent 49% of the RRCF dataset.

This observation implies that there is an underlying biological feature of CD4+ T cells from donors associated with GVHD positive outcomes, i.e., that gene expression levels are generally substantially higher for the vast majority of genes in CD4+ T cells from donors that cause GVHD, compared to donors associated with GVHD negative outcomes. This may be potentially due to elevated metabolic and transcriptional activity in more alloreactive CD4+ T cells; however, in-depth studies of such differences in metabolic activity do not appear in the scientific literature.

Given how well SGs from RL2F data perform on the individual SG level with respect to GVHD outcome prediction, they may also perform well in the types of SG Vmods examined above. However, as observed in Table 31 (RL2FRRCFSGVmodcomp), RL2F data perform very poorly compared to RRCF data for the 43 SG implementation of Vmod SG43RGP36-RGPgreedysearch.

Clearly, on the SG level, RL2F data should not be substituted for RRCF data in the Vmods selected above for GVHD outcome prediction applications, as suggested in Table 31 for the 43 SG implementation of Vmod SG43RGP36-RGPgreedysearch. However, given SG prioritizations especially selected for RL2F data (other than involving ratiometric or other self-calibrating methods as used above), it is conceivable that RL2F SG Vmods could be designed with higher GVHD outcome predictive performance than the currently examined version of an RL2F SG Vmod. Given the drawbacks inherent to laboratory measurement reliability of difficult to calibrate absolute RL2F RT-PCR data, designing a GVHD outcome prediction test based on RL2F data, while possible in-principle, may be risky with respect to reliability, and therefore although not further pursued, may become a priority for development.

TABLE 30
Comparison of GVHD outcome predictive performance for SGs based on RL2F (absolute quantitation)
and RRCF (relative quantitation) RT-PCR data (“RL2FRRCFSGcomp”).

| | | Gneg vs. Gpos | Gneg vs. Gag2 | Gneg vs. Gag3 | SG % | SG % |
|---|---|---|---|---|---|---|
| Data type | Performance variable | T-test p-value | T-test p-value | T-test p-value | P-directional | N-directional |
| RL2F (absolute gene expression) | SG min | 0.0004 | 0.0001 | 0.0013 | 97% | 3% |
| RL2F (absolute gene expression) | SG max | 0.9346 | 0.9923 | 0.9308 | | |
| RL2F (absolute gene | SG geometric mean | 0.0830 | 0.0458 | 0.1165 | | |
| expression) | | | | | | |
| RL2F (absolute gene | SG p-value % <=0.05 | 61 | 88 | 45 | | |
| expression) | | | | | | |
| RRCF (relative gene expression) | SG minimum | 0.0002 | 0.0001 | 0.0001 | 49% | 51% |
| RRCF (relative gene expression) | SG maximum | 0.9877 | 0.9997 | 0.9881 | | |
| RRCF (relative gene expression) | SG geometric mean | 0.1827 | 0.1787 | 0.1641 | | |
| RRCF (relative gene expression) | SG p-value % <=0.05 | 32 | 32 | 32 | | |

TABLE 31
Comparison of GVHD outcome predictive performance for SG Vmods based on RL2F (absolute
quantitation) and RRCF (relative quantitation) RT-PCR data (“RL2FRRCFSGVmodcomp”).

| | | Gneg vs. | | NPV | TNR Gneg | TPR Gneg | | GVHD |
|---|---|---|---|---|---|---|---|---|
| | | Gpos T-test | GNOS | Gneg vs. | vs. Gag3 | vs. Gag3 | GVHD | N donor |
| Data type | Vmod | p-value | threshold | Gag3 | (specificity) | (sensitivity) | reduction | capture |
| RL2F (relative | 43 SG Vmod from | 1.12E−02 | 0.50 | 0.82 | 0.53 | 0.66 | 29% | 53% |
| gene expression) | SG43RGP36- | | | | | | | |
| | RGPgreedysearch | | | | | | | |
| RL2F (relative | 43 SG Vmod from | 1.12E−02 | 0.55 | 0.82 | 0.51 | 0.66 | 28% | 51% |
| gene expression) | SG43RGP36- | | | | | | | |
| | RGPgreedysearch | | | | | | | |
| RL2F (relative | 43 SG Vmod from | 1.12E−02 | 0.65 | 0.83 | 0.47 | 0.70 | 31% | 47% |
| gene expression) | SG43RGP36- | | | | | | | |
| | RGPgreedysearch | | | | | | | |
| RL2F (relative | 43 SG Vmod from | 1.12E−02 | 0.75 | 0.82 | 0.36 | 0.77 | 28% | 36% |
| gene expression) | SG43RGP36- | | | | | | | |
| | RGPgreedysearch | | | | | | | |
| RL2F (relative | 43 SG Vmod from | 1.12E−02 | 0.85 | 0.86 | 0.29 | 0.86 | 43% | 29% |
| gene expression) | SG43RGP36- | | | | | | | |
| | RGPgreedysearch | | | | | | | |
| RRCF (relative | 43 SG Vmod from | 3.80E−10 | 0.50 | 0.90 | 0.76 | 0.75 | 61% | 76% |
| gene expression) | SG43RGP36- | | | | | | | |
| | RGPgreedysearch | | | | | | | |
| RRCF (relative | 43 SG Vmod from | 3.80E−10 | 0.55 | 0.93 | 0.64 | 0.84 | 70% | 64% |
| gene expression) | SG43RGP36- | | | | | | | |
| | RGPgreedysearch | | | | | | | |
| RRCF (relative | 43 SG Vmod from | 3.80E−10 | 0.65 | 0.96 | 0.37 | 0.95 | 82% | 37% |
| gene expression) | SG43RGP36- | | | | | | | |
| | RGPgreedysearch | | | | | | | |
| RRCF (relative | 43 SG Vmod from | 3.80E−10 | 0.75 | 1.00 | 0.03 | 1.00 | 100% | 3% |
| gene expression) | SG43RGP36- | | | | | | | |
| | RGPgreedysearch | | | | | | | |
| RRCF (relative | 43 SG Vmod from | 3.80E−10 | 0.85 | — | 0.00 | 1.00 | — | 0% |
| gene expression) | SG43RGP36- | | | | | | | |
| | RGPgreedysearch | | | | | | | |

### Example 18

This example includes a discussion of the rank order of GNOS values in GVHD outcome predictive groups reflecting increasing severity of GVHD.

The GVHD groups analyzed here reflect varying intensities of GVHD, from Gneg, i.e. no GVHD, to Gag3, i.e., severe and often fatal acute grades III or IV GVHD, and various disease intensity gradations in-between. Specifically, the GVHD outcome classes cover 6 different groups (not to be confused with the Groups listed above), in a medically-accepted order of GVHD severity, as follows:

(1) Gneg (no acute nor chronic GVHD),

(2) cG only (chronic GVHD without acute GHVHD),

(3) ag2 (acute grade II GVHD, without acute grade III or IV GVHD, with or without chronic GVHD)

(4) Gpos (any kind of GVHD, including chronic and acute grades II, III or IV GVHD)

(5) Gag2 (acute grade II, III or IV GVHD, with or without chronic GVHD), and

(6) Gag3 (acute grade III or IV GVHD, with or without chronic GVHD).

For the samples within each of these 6 groups, the GNOS values were averaged for three different Vmods, from data in the presence of varying amounts of added numerical noise (see above, “Robustness of Vmod outcome prediction from RT-PCR and microarray RGP data in the presence of noise”). From these GNOS averages, ranks were determined in descending order, i.e., the highest GNOS average is ranked as 1.0, and the lowest GNOS average is ranked as 6.0.

Significantly, in Table 32 (GNOSrankorder), for the best performing Vmods SG43RGP36-RGPgreedysearch and SG21RGP28-RGPmaxgreedysearch, and in close approximation for Vmod SG43RGP46-RGPperformance, we consistently observe the same rank order of GNOS averages as we do for the medically-accepted order of disease severity listed above. This consistently applies to the RT-PCR and to the microarray data used as inputs for these Vmods. Even in the presence of up to 2×s.d. of noise added to the data, the Gneg and Gag3 groups consistently reflect the extreme ranks, and the other groups generally fall in-between in the order listed above.

In conclusion, the GNOS values, as reflected in the ranks of the 6 GVHD group averages, therefore very highly likely reflects an inherent, integrated genuine biological signal that varies in direct proportion to the severity of GVHD, as also indicated in the medically-accepted order of GVHD severity listed above. This reflection of an integrated underlying biological signal is robust with respect to whether different Vmods were used, whether RT-PCR or microarray data were used, and whether slight to extreme levels of numerical random noise were added to the measurement data. Thus, outcome prediction of recipient GVHD from donor CD4+ cell gene expression profiles is fundamentally due to complex biological patterns of gene activation and repression in these cells, which vary in direction proportion to the severity of recipient GVHD, and are informationally captured in the exemplified voting models of ratiometric gene pairs disclosed herein.

TABLE 32
Comparison of rank order of average GNOS values for 6 different GVHD outcome groups, using RT-
PCR and microarray data, in the presence of various levels of noise (“GNOSrankorder”).

| | | Vmod | | | | | |
|---|---|---|---|---|---|---|---|
| | | SG43RGP46- | SG43RGP46- | SG43RGP46- | SG43RGP46- | SG43RGP46- | SG43RGP46- |
| | Noise s.d. | RGPperformance | RGPperformance | RGPperformance | RGPperformance | RGPperformance | RGPperformance |
| | scaling | Rank Gneg | Rank Gpos | Rank Gag2 | Rank Gag3 | Rank cG only | Rank aGg2 |
| Data type | factor | average | average | average | average | (no aG) average | (no aGg34) average |
| RRCF (RT-PCR) | 0.1 | 1.0 | 4.0 | 5.0 | 6.0 | 2.1 | 2.9 |
| RRCF (RT-PCR) | 0.2 | 1.0 | 4.0 | 5.0 | 6.0 | 2.3 | 2.7 |
| RRCF (RT-PCR) | 0.5 | 1.0 | 4.0 | 5.0 | 6.0 | 2.3 | 2.7 |
| RRCF (RT-PCR) | 1.0 | 1.0 | 4.0 | 4.8 | 6.0 | 2.6 | 2.6 |
| RRCF (RT-PCR) | 2.0 | 1.0 | 4.0 | 4.5 | 5.6 | 3.2 | 2.8 |
| RRCF (RT-PCR) | 5.0 | 1.4 | 3.9 | 4.1 | 4.7 | 3.6 | 3.3 |
| RRCF (RT-PCR) | 10.0 | 2.1 | 3.8 | 3.9 | 4.2 | 3.6 | 3.3 |
| VQLS (microarray) | 0.1 | 1.0 | 4.0 | 3.6 | 5.5 | 4.8 | 2.0 |
| VQLS (microarray) | 0.2 | 1.0 | 4.0 | 3.7 | 5.4 | 4.8 | 2.2 |
| VQLS (microarray) | 0.5 | 1.0 | 4.1 | 3.6 | 4.9 | 4.8 | 2.6 |
| VQLS (microarray) | 1.0 | 1.0 | 4.1 | 3.7 | 4.3 | 4.5 | 3.3 |
| VQLS (microarray) | 2.0 | 1.0 | 4.0 | 3.9 | 4.1 | 4.2 | 3.8 |
| VQLS (microarray) | 5.0 | 1.2 | 4.0 | 3.9 | 4.0 | 4.1 | 3.8 |
| VQLS (microarray) | 10.0 | 1.9 | 3.9 | 3.8 | 3.9 | 3.8 | 3.7 |
| | | Vmod | | | | | |
| | | SG43RGP36- | SG43RGP36- | SG43RGP36- | SG43RGP36- | SG43RGP36- | SG43RGP36- |
| | Noise s.d. | RGPgreedysearch | RGPgreedysearch | RGPgreedysearch | RGPgreedysearch | RGPgreedysearch | RGPgreedysearch |
| | scaling | Rank Gneg | Rank Gpos | Rank Gag2 | Rank Gag3 | Rank cG only | Rank aGg2 |
| Data type | factor | average | average | average | average | (no aG) average | (no aGg34) average |
| RRCF (RT-PCR) | 0.1 | 1.0 | 4.0 | 5.0 | 6.0 | 2.0 | 3.0 |
| RRCF (RT-PCR) | 0.2 | 1.0 | 4.0 | 5.0 | 6.0 | 2.1 | 2.9 |
| RRCF (RT-PCR) | 0.5 | 1.0 | 4.0 | 5.0 | 6.0 | 2.4 | 2.7 |
| RRCF (RT-PCR) | 1.0 | 1.0 | 4.0 | 4.7 | 5.8 | 2.8 | 2.7 |
| RRCF (RT-PCR) | 2.0 | 1.0 | 4.0 | 4.3 | 5.1 | 3.4 | 3.2 |
| RRCF (RT-PCR) | 5.0 | 1.5 | 3.9 | 4.1 | 4.5 | 3.4 | 3.5 |
| RRCF (RT-PCR) | 10.0 | 2.2 | 3.8 | 3.9 | 4.1 | 3.5 | 3.5 |
| VQLS (microarray) | 0.1 | 1.0 | 3.9 | 5.0 | 6.0 | 2.0 | 3.1 |
| VQLS (microarray) | 0.2 | 1.0 | 3.9 | 5.0 | 5.9 | 2.0 | 3.2 |
| VQLS (microarray) | 0.5 | 1.0 | 3.9 | 4.9 | 5.7 | 2.4 | 3.2 |
| VQLS (microarray) | 1.0 | 1.0 | 3.9 | 4.5 | 5.2 | 3.1 | 3.3 |
| VQLS (microarray) | 2.0 | 1.0 | 3.9 | 4.3 | 4.6 | 3.5 | 3.7 |
| VQLS (microarray) | 5.0 | 1.4 | 4.0 | 4.0 | 4.2 | 3.7 | 3.7 |
| VQLS (microarray) | 10.0 | 2.1 | 3.8 | 3.9 | 4.0 | 3.6 | 3.6 |

| | | Vmod | | | | |
|---|---|---|---|---|---|---|
| | Noise | SG21RGP28- | SG21RGP28- | SG21RGP28- | SG21RGP28- | SG21RGP28- |
| | s.d. | RGPmaxgreedysearch | RGPmaxgreedysearch | RGPmaxgreedysearch | RGPmaxgreedysearch | RGPmaxgreedysearch |
| | scaling | Rank Gneg | Rank Gpos | Rank Gag2 | Rank Gag3 | Rank cG only |
| Data type | factor | average | average | average | average | (no aG) average |
| RRCF (RT-PCR) | 0.1 | 1.0 | 4.0 | 5.0 | 6.0 | 2.0 |
| RRCF (RT-PCR) | 0.2 | 1.0 | 4.0 | 5.0 | 6.0 | 2.0 |
| RRCF (RT-PCR) | 0.5 | 1.0 | 4.0 | 5.0 | 6.0 | 2.3 |
| RRCF (RT-PCR) | 1.0 | 1.0 | 3.9 | 4.7 | 5.7 | 2.7 |
| RRCF (RT-PCR) | 2.0 | 1.1 | 3.9 | 4.4 | 5.0 | 3.2 |
| RRCF (RT-PCR) | 5.0 | 1.7 | 3.9 | 4.1 | 4.4 | 3.4 |
| RRCF (RT-PCR) | 10.0 | 2.4 | 3.8 | 3.8 | 3.9 | 3.5 |
| VQLS (microarray) | 0.1 | 1.0 | 4.0 | 5.0 | 6.0 | 2.0 |
| VQLS (microarray) | 0.2 | 1.0 | 3.9 | 5.0 | 6.0 | 2.0 |
| VQLS (microarray) | 0.5 | 1.0 | 3.9 | 4.8 | 5.8 | 2.5 |
| VQLS (microarray) | 1.0 | 1.0 | 3.9 | 4.4 | 5.3 | 3.3 |
| VQLS (microarray) | 2.0 | 1.1 | 4.0 | 4.2 | 4.7 | 3.7 |
| VQLS (microarray) | 5.0 | 1.6 | 3.9 | 4.0 | 4.3 | 3.7 |
| VQLS (microarray) | 10.0 | 2.5 | 3.7 | 3.8 | 4.0 | 3.6 |

| | | Vmod |
|---|---|---|
| | | SG21RGP28-RGPmaxgreedysearch |
| Data type | Noise s.d. scaling factor | Rank aGg2 (no aGg34) average |
| RRCF (RT-PCR) | 0.1 | 3.0 |
| RRCF (RT-PCR) | 0.2 | 3.0 |
| RRCF (RT-PCR) | 0.5 | 2.8 |
| RRCF (RT-PCR) | 1.0 | 3.0 |
| RRCF (RT-PCR) | 2.0 | 3.4 |
| RRCF (RT-PCR) | 5.0 | 3.6 |
| RRCF (RT-PCR) | 10.0 | 3.5 |
| VQLS (microarray) | 0.1 | 3.0 |
| VQLS (microarray) | 0.2 | 3.1 |
| VQLS (microarray) | 0.5 | 3.0 |
| VQLS (microarray) | 1.0 | 3.1 |
| VQLS (microarray) | 2.0 | 3.4 |
| VQLS (microarray) | 5.0 | 3.5 |
| VQLS (microarray) | 10.0 | 3.5 |

### Example 19

This example includes a discussion of considering multiple options of gene and voting model selection with potential for high outcome predictive performance and high likelihood of validation.

Any GVHD outcome predictive single classifier or voter, independent of how the gene expression data, RT-PCR or microarray based, was processed at the single gene, gene pair, or integrated voting model level (e.g., RL2F, RRCF, VQLS, SG, RGP, GNOS, etc.) results in a continuous classifier level (CL), for each sample to be classified. When the CL average for the Gneg samples is higher than for the Gpos samples, the classifier is considered N-directional, or N_{d} (N for GVHD negative). When the CL average for the Gpos samples is higher than for the Gneg samples, the classifier is considered P-directional, or P_{d} (P for GVHD positive). The midpoint between the respective CL averages for the Gneg samples and Gpos samples is defined as the separatrix for each CL. For N_{d} classifiers, when the CL is higher than or equal to the separatrix, a GVHD N vote is cast, represented by the value 1; otherwise the vote value is set to 0. For P_{d} classifiers, when the CL is lower than the separatrix, a GVHD N vote is cast, represented by the value 1; otherwise the vote value is set to 0.

The GVHD N outcome votes of any set of classifiers, from very many potential combinations of the classifiers listed above, can be integrated into a voting model (Vmod). The voting models, as described herein, simply form the average of the GVHD N votes, which is called the GNOS (GVHD Negative Outcome Score). However, voters and classifiers can be integrated using other approaches that would lead to dependable GVHD outcome prediction (see below, “Alternatives for multivariate outcome predictive models”). Because the GNOS is defined herein solely on “GVHD N” votes being set to 1, and “not-GVHD N” votes being set to 0, the GNOS-based classifiers are always N-directional. Also, when determining the final GVHD outcome classification of a sample according to its GNOS, often an N-voting threshold (e.g., 55% for the best-performing SG43RGP36-RGPgreedysearch), other than the separatrix, is selectively imposed according to desired GVHD outcome prediction performance goals.

Directionalities of GVHD Outcome Predictive Classifiers:

In general, using the genes in Table 13 (RNA175), or from Table 2B (RNA192 list) (note that all the Table 13 RNA175 genes are also listed in the Table 2B RNA192 list), or from the RNA1546 or RNA1538 lists (note that not all the RNA175 genes in Table 13 are listed in the RNA1546 or RNA1538 lists), multiple, almost unlimited (based on different combinatorial subsets of ratiometric gene pairs as shown above and in general, or gene pairs in general, or directly using SGs as classifiers, as reflected in, e.g., RL2F, RRCF, VQLS, SG, RGP, etc. data), Vmods for successful GVHD outcome prediction may be generated, and many validated, freely allowing for different combinations of N_{d} and P_{d} classifiers, i.e.,

(1) mixed N_{d} and P_{d} classifiers, with varying relative representations of N_{d} and P_{d} classifiers, or

(2) only using P_{d} classifiers, or

(3) only using N_{d} classifiers.

With respect to RGP Vmods, i.e., ratiometric gene pair voting models, based on relative SG measurements (whether using RT-PCR or microarray data), for the outcome predictive signal (X/Y or equivalent log [X/Y] or log X−log Y) to be usefully assayed in-lab at the gene pair level (in addition to the inherent self-calibrating properties of RGPs), in a vast majority of cases the directionality of the RGP member genes should be opposite, i.e., when gene X is N_{d}, then gene Y should be P_{d}, and when gene X is P_{d}, then gene Y should be N_{d}. This follows the interpretative biological reasoning that only when the “activator pathway” activity of gene X is higher relative to the “inhibitor pathway” activity of gene Y (and vice-versa), the biological response (e.g., due to relative pathway activation being sufficient) occurs.

In addition, there may be cases of layered competitive pathways, e.g., pathways X and Y may both be elevated in the absolute sense for the biological response, but, nevertheless, pathway X must be more elevated relative to pathway Y for the full biological response to take place. Thus, occasionally, for RGP-based outcome prediction, gene X-Y, P_{d}-P_{d} or N_{d}-N_{d} pairs may occur, i.e., when P_{d} and N_{d} directionality is defined at the SG level for relative quantitation RTPCR or microarray data. However, overall, the RGPs contributing to RGP voting models should be fairly evenly balanced with respect to numbers of SGs having P_{d} or N_{d} status at the SG level (for relatively quantified gene expression data).

However, with respect to RGP Vmods based on RL2F absolute SG measurements, because the vast majority of RL2F genes are biased toward P-directionality (see above), mostly RGP X-Y, P_{d}-P_{d} pairs would be used as outcome predictive classifiers to go into the Vmods. Again, effective RGP values as such are not dependent, though, on whether RRCF or RL2F data are used as input.

With respect to Vmods using SGs as constituent classifiers, according to basic principles, no favored SG P- or N-directionalities are required for Vmods to be effective, especially when using relative quantitation of gene expression data, e.g., in the form of RRCF. Also note that the SGs in Table 13 (RNA175; as determined from RRCF and VQLS data are relatively evenly balanced with respect to directionality (see also Table 30).

However, as discussed above, when using absolute as opposed to relative RT-PCR quantitation RL2F data for GVHD outcome prediction, there is a dominating natural inherent bias towards prevalence of P-directional genes in SG ability for GVHD outcome prediction (according to the biological trends displayed in the data). Thus, at the level of absolute quantitation, RL2F-based GVHD outcome prediction, any potential well-performing voting models (which have not been explicitly listed), would most likely be based on a vast majority of P-directional SGs at the RL2F level. Such integrated P_{d} directional SG Vmods based on absolute RT-PCR quantitation might be very effective at GVHD outcome prediction, and possibly developed as a GVHD outcome prediction test. However, given the current practice in diagnostic applications of RT-PCR, in which absolute quantitation is not considered today to be a dependable assay for human diagnostics, SG Vmods based on absolute RT-PCR quantitation are not a present priority for development. However, such models may become a priority for development in the future.

### Example 20

This example includes a discussion of alternatives to the exemplified Vmods disclosed herein for multivariate outcome predictive models.

Note that aggregating and averaging a select set of individual RGP votes into a GNOS value is one of the most straightforward ways to efficiently, pragmatically, robustly, and transparently use the information in individual mRNA measurement levels of multiple genes to provide a GVHD N outcome score. However, many alternative methods (generally referred to as classifiers) exist to generate multivariate predictive models, in addition to multi-RGP Vmods. Such alternative classifiers (Richard O. Duda, Peter E. Hart, & David G. Stork, Pattern Classification, Second Edition, John Wiley & Sons, Inc, NY, 2001) include those built on weighted averages of individual variables, weighted averages of pair-wise combinations of variables, or weighted averages of multivariate combinations of variables, linear or non-linear, such as could be implemented in LDA (linear discriminant analysis), QDA (quadratic linear discriminant analysis), Decision Trees, SVMs (support vector machines), k-nearest neighbors, Neural Networks, etc., or various implementations of generalized multivariate linear and nonlinear models, with varying degrees of freedom, coupled with judicious search and optimization algorithms (e.g., classical optimization algorithms or derivative-free algorithms such as so-called genetic algorithms) Such alternative methods may be used to derive GNOS values from the lists of SGs, RGPs and PRGPs, listed herein. However, depending on the comparative complexity and degrees of freedom of such models, more observational combined donor gene expression measurement and associated recipient GVHD clinical outcome data samples may be required to provide adequate statistical support of such alternative, more complex implementations of classifiers.

What is claimed:

1. A method for predicting or determining the risk of a hematopoietic cell transplant (HCT) from a candidate donor to induce or not to induce graft vs. host disease (GVHD) in a HCT recipient, comprising:
a) measuring expression of one or more positive or negative GVHD predictor genes, or a combination of positive and/or negative GVHD predictor genes, selected from Tables 1 (RNA 1538), 2, 2A (RNA 143), 2B (RNA 192), 3, 13 (SG175), 15 (SG128) or 18 (SG64), or a polymorphism thereof, in CD4+ T cells or CD8+ T cells from a candidate donor;
b) obtaining an expression value for the positive or negative GVHD predictor genes based upon the expression measured in a), or obtaining linear or non-linear combinations of expression values for the combination of positive and/or negative GVHD predictor genes based upon the expression measured in a);
c) comparing the expression value for the positive or negative GVHD predictor gene to a predefined reference expression value for the positive or negative GVHD predictor gene, or comparing the linear or non-linear combinations of expression values for the combination of positive and/or negative GVHD predictor genes to predefined reference values for the linear or non-linear combinations of the positive and/or negative GVHD predictor genes;
wherein an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, or
wherein an expression value for the negative GVHD predictor gene greater or less than the reference expression value for the negative GVHD predictor gene indicates that the HCT from the candidate donor is at lower or higher risk, respectively, of inducing GVHD in a HCT recipient, or
wherein a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient; or
wherein a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient, and
d) predicting or determining the risk of the HCT from the candidate donor to induce or to not induce GVHD in an HCT recipient, based upon an evaluation of expression values, total numbers or identity of positive or negative GVHD predictor genes, or the combination of positive and/or negative GVHD predictor genes, that indicate that the HCT from the candidate donor is at higher or lower risk of inducing GVHD in a HCT recipient.

2. A method for predicting or determining the risk of a HCT from a candidate donor to induce or not to induce graft vs. host disease (GVHD) in a HCT recipient, comprising:
a) contacting CD4+ T cells or CD8+ T cells, or nucleic acid or protein expressed by CD4+ T cells or CD8+ T cells, from a candidate donor with an analyte that detects expression of one or more positive or negative GVHD predictor genes listed in Tables 1 (RNA 1538), 2, 2A (RNA 143), 2B (RNA 192), 3, 13 (SG175), 15 (SG128) or 18 (SG64), or a polymorphism thereof;
b) measuring expression of the one or more positive or negative GVHD predictor genes in CD4+ T cells or CD8+ T cells to obtain an expression value for the positive or negative GVHD predictor genes, or measuring expression of a combination of the positive and/or negative predictor genes to obtain linear or non-linear combinations of expression values for the combination of positive and/or negative GVHD predictor genes;
c) comparing the expression value for the positive or negative GVHD predictor gene to a predefined reference expression value for the positive or negative GVHD predictor gene, or comparing the linear or non-linear combinations of expression values of the combination of positive and/or negative GVHD predictor genes to a predefined reference value for the linear or non-linear combinations of expression values of the combination of positive and/or negative GVHD predictor genes;
wherein an expression value for the positive GVHD predictor gene greater or less than the predefined reference expression value for the positive GVHD predictor gene indicates that the HCT from the candidate donor is at higher or lower risk, respectively, of inducing GVHD in a HCT recipient,
wherein an expression value for the negative GVHD predictor gene greater or less than the reference expression value for the negative GVHD predictor gene indicates that the HCT from the candidate donor is at lower or higher risk, respectively, of inducing GVHD in a HCT recipient, or
wherein a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient,
wherein a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient, and
d) predicting or determining the risk of the HCT from the candidate donor to induce or to not induce GVHD in a HCT recipient, based upon an evaluation of expression values, total numbers or identity of positive or negative GVHD predictor genes, or combination of positive and/or negative GVHD predictor genes, that indicate that the HCT from the candidate donor is at higher or lower risk of inducing GVHD in a HCT recipient.

3. A method for classifying a hematopoietic cell transplant (HCT) from a candidate donor for risk of inducing graft vs. host disease (GVHD) in a HCT recipient, comprising:
a) measuring expression of a plurality of positive or negative GVHD predictor genes selected from a gene listed in Tables 1 (RNA 1538), 2, 2A (RNA 143), 2B (RNA 192), 3, 13 (SG175), 15 (SG128) or 18 (SG64), or a polymorphism thereof, in CD4+ T cells or CD8+ T cells from the candidate HCT donor
b) obtaining an expression value for the positive or negative GVHD predictor genes based upon the expression measured in a), or obtaining linear or non-linear combinations of expression values for the combination of positive and/or negative GVHD predictor genes based upon the expression measured in a);
c) comparing the expression value for the positive or negative GVHD predictor gene to a predefined reference expression value for the positive or negative GVHD predictor gene, or comparing the linear or non-linear combinations of expression values for the combination of positive and/or negative GVHD predictor genes to predefined reference values for the linear or non-linear combinations of the positive and/or negative GVHD predictor genes;
wherein an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, or
wherein an expression value for the negative GVHD predictor gene greater or less than the reference expression value for the negative GVHD predictor gene indicates that the HCT from the candidate donor is at lower or higher risk, respectively, of inducing GVHD in a HCT recipient, or
wherein a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient; or
wherein a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient; and
d) classifying the candidate donor HCT for risk of inducing or not inducing GVHD based upon an evaluation of expression values, total numbers or identity of positive or negative GVHD predictor genes, or combination of positive and/or negative GVHD predictor genes, that indicate that the HCT from the candidate donor is at higher or lower risk of inducing GVHD in a HCT recipient.

4. A method of producing a database or organizational construct comprising a plurality of actual or candidate HCT donors each assigned a score based upon the probability or degree of risk of the actual or candidate donor HCT to induce or not to induce graft vs. host disease (GVHD) in a HCT recipient, comprising:
a) measuring expression of one or more positive or negative GVHD predictor genes listed in Tables 1 (RNA 1538), 2, 2A (RNA 143), 2B (RNA 192), 3, 13 (SG175), 15 (SG128) or 18 (SG64), or a polymorphism thereof, in CD4+ T cells or CD8+ T cells from an actual or a candidate donor;
b) obtaining an expression value for the positive or negative GVHD predictor genes based upon the expression measured in a), or obtaining linear or non-linear combinations of expression values for the combination of positive and/or negative GVHD predictor genes based upon the expression measured in a);
c) comparing the expression value for the positive or negative GVHD predictor gene to a predefined reference expression value for the positive or negative GVHD predictor gene, or comparing the linear or non-linear combinations of expression values for the combination of positive and/or negative GVHD predictor genes to predefined reference values for the linear or non-linear combinations of the positive and/or negative GVHD predictor genes;
wherein an expression value for the positive GVHD predictor gene greater or less than the predefined reference expression value for the positive GVHD predictor gene indicates that the HCT from the candidate donor is at higher or lower risk, respectively, of inducing GVHD in a HCT recipient, or
wherein an expression value for the negative GVHD predictor gene greater or less than the reference expression value for the negative GVHD predictor gene indicates that the HCT from the candidate donor is at lower or higher risk, respectively, of inducing GVHD in a HCT recipient, or
wherein a linear or non-linear combination of expression values for the combination of positive and/or negative GVHD predictor genes greater or less than the predefined reference value indicates that the HCT from the candidate donor is at higher or lower risk, respectively, of inducing GVHD in a HCT recipient; or
wherein a linear or non-linear combination of expression values for the combination of positive and/or negative GVHD predictor genes greater or less than the predefined reference value indicates that the HCT from the candidate donor is at lower or higher risk, respectively, of inducing GVHD in a HCT recipient;
d) assigning a score based upon an evaluation of expression values, total numbers or identity of positive or negative GVHD predictor genes, or combination of positive and/or negative GVHD predictor genes, that indicate that the HCT from the candidate donor is at higher or lower risk of inducing GVHD in a HCT recipient, wherein the score reflects the probability or degree of risk of the actual or candidate donor HCT to induce GVHD in a HCT recipient,
e) recording or storing the score of the actual or candidate HCT donor; and
f) repeating steps a), b), c), d) and e) for one or more additional actual or candidate HCT donors,
thereby producing a database or organizational construct comprising actual or candidate HCT donors each assigned a score based upon the probability or degree of risk of the actual or candidate donor HCT to induce or to not induce graft vs. host disease (GVHD) in a HCT recipient.

5. The method of any of claim 1, 2 or 4, comprising measuring expression of a plurality of positive or negative predictor genes to obtain expression values for the plurality of positive or negative predictor genes, and comparing the expression value for the positive or negative predictor genes to a predefined reference expression value for the respective positive or negative predictor genes.

6. The method of any of claims 1 to 4, wherein the positive or negative predictor gene is selected from one or more positive or negative predictor genes listed in Tables 2A (RNA 143), 2B (RNA 192), 3, 13 (SG175), 15 (SG128), or 18 (SG64), or a polymorphism thereof.

7. The method of claim 6, wherein the plurality of positive or negative predictor genes measured is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more positive or negative predictor genes.

8. The method of any of claims 1 to 4, wherein an expression value for the positive predictor gene greater than the predefined reference expression value for the positive predictor gene correlates with expression of the positive predictor gene in one or more HCT donors known to induce GVHD.

9. The method of any of claims 1 to 4, wherein an expression value for the negative predictor gene greater than the predefined reference expression value for the negative predictor gene correlates with expression of the negative predictor gene in one or more HCT donors known not to induce GVHD.

10. The method of any of claims 1 to 4, wherein the predefined reference expression value for the positive predictor gene is midway between an average or median expression level of the positive predictor gene from two or more HCT donors that induce GVHD and two or more HCT donors that do not induce GVHD.

11. The method of any of claims 1 to 4, wherein the predefined reference expression value for the negative predictor gene is midway between an average or median expression level of the negative predictor genes from two or more HCT donors that induce GVHD and two or more HCT donors that do not induce GVHD.

12. The method of any of claims 1 to 4, wherein the predefined reference expression value for the positive or negative predictor gene is midway between an average or median expression level of the positive or negative predictor genes from at least 5 HCT donors that induce GVHD and at least 5 HCT donors that do not induce GVHD.

13. The method of any of claims 1 to 4, wherein the predefined reference expression value for the positive predictor gene is midway between a median or average expression of the gene from multiple HCT donors known to induce GVHD, and a median or average expression of the gene from multiple HCT donors known not to induce GVHD.

14. The method of any of claims 1 to 4, wherein the predefined reference expression value for the negative predictor gene is midway between the median or average expression of the gene from multiple HCT donors known to induce GVHD, and the median or average expression of the gene from multiple HCT donors known not to induce GVHD.

15. The method of any of claims 1 to 4, wherein the predefined reference expression value for the positive predictor gene is a midway value, midway between the expression level of the positive predictor gene from one or more donors that induce GVHD and the expression level of the positive predictor gene from one or more donors that do not induce GVHD, and wherein the expression value for the positive predictor gene greater than the midway value indicates that the HCT from the candidate donor is at higher risk of inducing graft vs. host disease (GVHD).

16. The method of any of claims 1 to 4, wherein the predefined reference expression value for the negative predictor gene is a midway value, midway between the expression level of the negative predictor gene from one or more donors that do not induce GVHD and the expression level of the negative predictor gene from one or more donors that induce GVHD, and wherein the expression value for the negative predictor gene greater than the midway value indicates that the HCT from the candidate donor is at lower risk of inducing graft vs. host disease (GVHD).

17. The method of any of claims 10 to 16, wherein the midway value is assigned a value of 0.5, and an expression value for the one or more negative predictor genes greater than 0.5 indicates that the HCT from the candidate donor is at lower risk of inducing graft vs. host disease (GVHD).

18. The method of any of claims 10 to 16, wherein the midway value is assigned a value of 0.5, and an expression value for the one or more negative predictor genes of 0.55 or greater indicates that the HCT from the candidate donor is at lower risk of inducing graft vs. host disease (GVHD).

19. The method of any of claims 10 to 16, wherein the midway value is assigned a value of 0.5, and an expression value for the one or more negative predictor genes of 0.60 or greater indicates that the HCT from the candidate donor is at lower risk of inducing graft vs. host disease (GVHD).

20. The method of any of claims 10 to 16, wherein the midway value is assigned a value of 0.5, and an expression value for the one or more positive predictor genes greater than 0.5 indicates that the HCT from the candidate donor is at higher risk of inducing graft vs. host disease (GVHD).

21. The method of any of claims 10 to 16, wherein the midway value is assigned a value of 0.5, and an expression value for the one or more positive predictor genes of 0.55 or greater indicates that the HCT from the candidate donor is at higher risk of inducing graft vs. host disease (GVHD).

22. The method of any of claims 10 to 16, wherein the midway value is assigned a value of 0.5, and an expression value for the one or more positive predictor genes of 0.60 or greater indicates that the HCT from the candidate donor is at higher risk of inducing graft vs. host disease (GVHD).

23. The method of any of claims 1 to 4, wherein the predefined reference expression value for the positive or negative predictor genes is a value determined by discriminant analysis of gene expression in HCT donors known to induce GVHD and HCT donors known not to induce GVHD.

24. The method of any of claims 1 to 4, wherein the expression value obtained for the positive or negative predictor genes is adjusted or normalized relative to expression of one or more reference genes prior to comparing the expression value of the positive or negative predictor gene to the predefined reference expression value for the positive or negative predictor gene.

25. The method of any of claims 1 to 4, wherein the expression value is represented by a ratio of gene expression, denoted a ratiometric gene pair (RGP), of the positive or negative predictor gene to one or more reference genes.

26. The method of any of claims 1 to 4, wherein the expression value is represented by a ratio of gene expression, denoted a ratiometric gene pair (RGP), of the positive or negative predictor gene to a reference gene, and is represented by the formula “N/D,” wherein “N” is the expression level of the positive or negative predictor gene, and “D” is the expression level of one or more reference genes.

27. The method of claim 26, wherein the numerator value N or denominator value D reflect an average or median expression of one or more positive or negative predictor genes, or one or more reference genes, respectively.

28. The method of any of claims 1 to 4, wherein the expression value is represented by a ratio of gene expression, denoted a ratiometric gene pair (RGP), of the positive or negative predictor gene to a reference gene, and expression of the positive or negative predictor gene, when expressed in log, is represented by the formula “logₙX−logₙY,” wherein “X” is the expression level of the positive or negative predictor gene, “Y” is the expression level of the reference gene, and “n” is 2, 10, e (base of natural log) or any positive real number.

29. The method of claim 25, 26 or 28, wherein at least one of the genes comprising the ratiometric gene pair (RGP) are listed in Tables 2A (RNA 143), 2B (RNA 192), 3, 13 (SG175), 15 (SG128), or 18 (SG64), or a polymorphism thereof.

30. The method of claim 24, 25, 26 or 28, wherein the reference gene comprises a positive or negative predictor gene that is different from the positive or negative predictor gene used to obtain the ratio of gene expression.

31. The method of claim 25, 26 or 28, wherein at least one of the ratiometric gene pairs (RGPs) is selected from the RGPs set forth in Table 14 (RGP348).

32. The method of claim 25, 26 or 28, wherein the number of gene expression ratios measured is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more gene expression ratios.

33. The method of any of claim 25, 26 or 28, wherein at least one of the positive or negative predictor genes that comprise the ratio are selected from one or more single genes (SGs) set forth in Tables 13 (SG175), 15 (SG128) or 18 (SG64), or are selected from ratiometric gene pairs (RGPs) set forth in Table 14 (RGP348).

34. The method of any of claim 24, 25, 26 or 28, wherein the reference gene comprises a gene whose expression is constitutive and at a relatively consistent level in CD4+ T cells or CD8+ T cells, a housekeeping gene, or a positive or negative predictor gene whose expression is not used to determine the numerator value.

35. The method of claim 34, wherein the housekeeping gene is selected from: beta actin (ACTB), aldolase A (ALDOA), lactate dehydrogenase A (LDHA), phosphoglycerate kinase 1 (PGK1), transferrin receptor (TFRC), tubulin beta (TUBB), tubulin beta 2A (TUBB2A), thioredoxin (TXN), ubiquitin C (UBC), ubiquitin-activating enzyme E1 (UBE1), a sequence in Table 2B (RNA 192, denoted HSK, SEQ ID NOs:1690-1738), or a sequence in Table 12 (HSK list).

36. The method of any of claims 1 to 4, wherein the positive or negative predictor gene is selected from Tables 13 (SG175), 15 (SG128) or 18 (SG64).

37. The method of any of claims 1 to 4, comprising a plurality of positive or negative predictor genes selected from Tables 13 (SG175), 15 (SG128) or 18 (SG64), or a plurality of ratiometric gene pairs (RGPs) selected from the RGPs set forth in Table 14 (RGP348).

38. The method of any of claims 1 to 4, comprising a plurality of positive and negative predictor genes selected from Tables 13 (SG175), 15 (SG128) or 18 (SG64), and a plurality of ratiometric gene pairs (RGPs) selected from the RGPs set forth in Table 14 (RGP348).

39. The method of any of claims 1 to 3, further comprising assigning a score based upon the expression value(s) for the positive or negative predictor gene(s), wherein the score reflects the probability or degree of risk of the candidate donor HCT to induce or not induce graft vs. host disease (GVHD) in a HCT recipient.

40. The method of any of claims 1 to 3, wherein a plurality of expression values for negative or positive predictor genes are determined, a vote is assigned to each negative or positive predictor gene according to whether the expression value for the gene indicates the risk of the candidate or actual donor to induce or not to induce GVHD, and a score is assigned to the candidate or actual donor based upon the total number of votes indicative or not indicative of inducing or not inducing GVHD, wherein the score reflects the risk of the hematopoietic cell transplant (HCT) from the candidate or actual donor to induce or not to induce GVHD in a HCT recipient.

41. The method of claim 40, wherein if more than 50% of the votes are indicative of inducing GVHD, then the score reflects an increased risk of the hematopoietic cell transplant (HCT) from the candidate or actual donor to induce GVHD in a HCT recipient.

42. The method of claim 40, wherein if more than 50% of the votes are indicative of not inducing GVHD, then the score reflects a decreased risk of the hematopoietic cell transplant (HCT) from the candidate or actual donor to induce GVHD in a HCT recipient.

43. The method of claim 40, wherein if at least 55%, 60%, 65%, 70%, 75%, 80%, 85%, 90%, 95%, or more of the votes are indicative of inducing GVHD, then the score reflects a increased risk of the hematopoietic cell transplant (HCT) from the candidate or actual donor to induce GVHD in a HCT recipient; or wherein if at least 55%, 60%, 65%, 70%, 75%, 80%, 85%, 90%, 95%, or more of the votes are indicative of not inducing GVHD, then the score reflects a decreased risk of the hematopoietic cell transplant (HCT) from the candidate or actual donor to induce GVHD in a HCT recipient.

44. The method of any of claims 1 to 3, wherein the number of positive or negative predictor genes, or the combination of positive and/or negative GVHD predictor genes, indicating that the HCT from the candidate donor is at higher risk of inducing GVHD is greater than the number of positive or negative predictor genes, or the combination of positive and/or negative GVHD predictor genes, indicating that the HCT from the candidate donor is at lower risk of inducing GVHD in a HCT recipient, predicts or determines a higher risk of the HCT from a candidate donor to induce GVHD in an HCT recipient.

45. The method of any of claims 1 to 3, wherein the number of positive or negative predictor genes, or the combination of positive and/or negative GVHD predictor genes, indicating that the HCT from the candidate donor is at lower risk of inducing GVHD is greater than the number of positive or negative predictor genes, or the combination of positive and/or negative GVHD predictor genes, indicating that the HCT from the candidate donor is at higher risk of inducing GVHD in a HCT recipient, predicts or determines a lower risk of the HCT from a candidate donor to induce GVHD in an HCT recipient.

46. The method of any of claims 1 to 4, wherein the negative and positive predictor genes used to predict or determine risk that a hematopoietic cell transplant (HCT) from a candidate donor will induce or not induce graft vs. host disease (GVHD) in a HCT recipient comprises one or more genes set forth in Table 18 (VmodSG64).

47. The method of any of claims 1 to 4, wherein the negative and positive predictor genes comprise a plurality of ratiometric gene pairs (RGPs) of two or more genes set forth in Tables 1 (RNA 1538), 2, 2A (RNA 143), 2B (RNA 192), 3, 13 (SG175), 15 (SG128) or 18 (SG64).

48. The method of claim 45, wherein the ratiometric gene pairs (RGPs) used to predict or determine risk that a hematopoietic cell transplant (HCT) from a candidate donor will induce or not induce graft vs. host disease (GVHD) in a HCT recipient comprise one or more gene pairs (RGPs) set forth in Table 17 (VmodRGP100).

49. The method of any of claims 1 to 4, wherein the negative and positive predictor genes comprise a combination of single genes (SGs) and ratiometric gene pairs (RGPs) used to predict or determine risk that a hematopoietic cell transplant (HCT) from a candidate donor will induce or not induce graft vs. host disease (GVHD) in a HCT recipient, wherein the combination comprises a plurality of genes selected from the single genes (SGs) listed in Table 18 (VmodSG64) and a plurality of ratiometric gene pairs (RGPs) selected from the RGPs listed in Table 17 (VmodRGP100).

50. The method of claim 49, wherein the combination of single genes (SGs) and ratiometric gene pairs (RGPs) is as set forth in: SG43RGP46-GPperformance; SG42RGP21-GPminimalist; SG43RGP37-GPconnectivity; SG43RGP51-PRGPminranksort; SG43RGP55-PRGPmedranksort; SG43RGP36-RGPgreedysearch; or SG21RGP28-RGPmaxgreedysearch, each of which combinations include the SGs and RGPs indicated by an “x” in Tables 17 and 18.

51. The method of any of claims 1 to 4, wherein the candidate donor and HCT recipient have 10 out of 10, or 9 out of 10, human leukocyte antigen (HLA) marker loci matches.

52. The method of any of claims 1 to 4, wherein the candidate donor and HCT recipient have HLA marker loci matches of all of: HLA-A, HLA-B, HLA-C, HLA-DRB1 and HLA-DQB1 loci, or have HLA marker loci matches of any four of: HLA-A, HLA-B, HLA-C, HLA-DRB1 or HLA-DQB1 loci.

53. The method of claim 51 or 52, wherein the HLA marker loci matches are determined serologically or by sequence analysis of HLA genes.

54. The method of any of claims 1 to 4, wherein the candidate donor and HCT recipient are not siblings or are not familially related.

55. The method of any of claims 1 to 4, wherein the candidate donor and HCT recipient are siblings or are familially related.

56. The method of any of any of claims 1 to 4, wherein the method is superior to identifying a GVHD negative donor based upon having 10 out of 10 or 9 out of 10 HLA marker loci matches with a HCT recipient.

57. The method of any of any of claims 1 to 4, wherein the method predicts GVHD negative donor HCT with an accuracy of at least 60%.

58. The method of any of claims 1 to 4, wherein the method predicts GVHD negative donor HCT with an accuracy of at least 70%.

59. The method of any of claims 1 to 4, wherein the method predicts GVHD negative donor HCT with an accuracy of at least 80%.

60. The method of any of claims 57 to 59, wherein the accuracy of predicting a GVHD negative donor is the probability or degree of risk of correctly identifying a GVHD negative donor within a group of candidate HCT donors classified as negative by 10 out of 10 HLA marker loci matches with an HCT recipient.

61. The method of any of claims 1 to 4, wherein the method predicts GVHD positive donor HCT with an accuracy of at least 60%.

62. The method of any of claims 1 to 4, wherein the method predicts GVHD positive donor HCT with an accuracy of at least 70%.

63. The method of any of claims 1 to 4, wherein the method predicts GVHD positive donor HCT with an accuracy of at least 80%.

64. The method of any of claims 1 to 4, wherein a threshold number of the positive or negative predictor genes must indicate a high risk of inducing graft vs. host disease (GVHD) in a HCT recipient to predict or determine that the candidate donor HCT is at high risk to induce graft vs. host disease (GVHD) in a HCT recipient.

65. The method of any of claims 1 to 4, wherein a threshold number of the positive or negative predictor genes must indicate a low risk of inducing graft vs. host disease (GVHD) in a HCT recipient to predict or determine that the candidate donor HCT is at low risk to induce graft vs. host disease (GVHD) in a HCT recipient.

66. The method of claim 64 or 65, further comprising assigning a score based upon the number of positive or negative predictor genes that indicate a high or a low risk of donor HCT inducing graft vs. host disease (GVHD) in a HCT recipient, wherein the score reflects the probability or degree of risk of the candidate donor HCT to induce graft vs. host disease (GVHD) in a HCT recipient.

67. The method of any of claims 1 to 4, wherein a majority of the positive or negative predictor genes must indicate a high risk of inducing graft vs. host disease (GVHD) in a HCT recipient to predict or determine that the candidate donor HCT is at high risk to induce graft vs. host disease (GVHD) in a HCT recipient.

68. The method of any of claims 1 to 4, wherein a majority of the positive or negative predictor genes must indicate a low risk of inducing graft vs. host disease (GVHD) in a HCT recipient to predict or determine that the candidate donor HCT is at low risk to induce graft vs. host disease (GVHD) in a HCT recipient.

69. The method of any of claims 1 to 4, wherein at least 66% of the positive or negative predictor genes must indicate a high risk of inducing graft vs. host disease (GVHD) in a HCT recipient to predict or determine that the candidate donor HCT is at high risk to induce graft vs. host disease (GVHD) in a HCT recipient.

70. The method of any of claims 1 to 4, wherein at least 66% of the positive or negative predictor genes must indicate a low risk of inducing graft vs. host disease (GVHD) in a HCT recipient to predict or determine that the candidate donor HCT is at low risk to induce graft vs. host disease (GVHD) in a HCT recipient.

71. The method of any of claims 1 to 4, wherein at least 75% of the positive or negative predictor genes must indicate a low risk of inducing graft vs. host disease (GVHD) in a HCT recipient to predict or determine that the candidate donor HCT is at low risk to induce graft vs. host disease (GVHD) in a HCT recipient.

72. The method of claim 2, wherein the analyte comprises a primer pair, an oligo- or poly-nucleotide probe, or an antibody or antigen binding fragment thereof.

73. The method of any of claims 1 to 4, wherein the measuring comprises hybridization with an oligo- or poly-nucleotide probe to RNA transcript produced from one of the positive or negative predictor genes, or a polymorphism thereof, or a cDNA derived from the RNA transcript of the positive or negative predictor gene, or a polymorphism thereof.

74. The method of any of claims 1 to 4, wherein the measuring comprises hybridization with an oligo- or poly-nucleotide probe or primer that hybridizes to a transcription product of a gene set forth in Tables 1 (RNA 1538), 2, 2A (RNA 143), 2B (RNA 192), 3, 13 (SG175), 15 (SG128) or 18 (SG64).

75. The method of any of claims 1 to 4, wherein the measuring comprises hybridization with an oligo- or poly-nucleotide probe or primer set forth in Tables 1 (RNA 1538), 2, 2A (RNA 143), 2B (RNA 192), 3, 13 (SG175), 15 (SG128) or 18 (SG64).

76. The method of any of claims 1 to 4, wherein the measuring comprises hybridization of a primer pair and subsequent amplification of a cDNA derived from the RNA transcript of the positive or negative predictor gene produced from the positive or negative predictor genes, or a polymorphism thereof.

77. The method of any of claims 1 to 4, wherein the primer pair is a pair set forth in sequence in Table 2B (RNA 192), or a primer pair that hybridizes to a transcript of a gene set forth in Tables 1 (RNA 1538), 2, 2A (RNA 143), 2B (RNA 192), 3, 13 (SG175), 15 (SG128) or 18 (SG64).

78. The method of any of claims 1 to 4, wherein the measuring comprises reverse transcription of RNA transcript to produce cDNA to determine expression levels of one or more positive or negative predictor genes.

79. The method of any of claims 1 to 4, wherein the CD4+ T cells or CD8+ T cells are from or are present in the candidate or actual donors' blood.

80. The method of any of claims 1 to 4, wherein the GVHD is classified as a group 1, 2, 3, 4, 5, or 6 class of GVHD.

81. The method of any of claims 1 to 4, wherein the GVHD is classified as acute grade I, II, III or IV GVHD, with or without chronic GVHD, or chronic GVHD without acute GVDH.

82. The method of any of claims 1 to 3, further comprising selecting a HCT donor at lower risk of inducing graft vs. host disease (GVHD) for a HCT recipient.

83. The method of any of claims 1 to 4, wherein the gene expression profile of candidate HCT or actual donors, or scores, or a risk profile of inducing or not inducing GVHD, are recorded or stored on a computer readable medium, electronic storage medium, or in a database or other organizational construct.

84. The method of any of claims 1 to 4, wherein candidate HCT donors with a low or a high risk to induce or to not induce graft vs. host disease (GVHD) are identified.

85. The method of any of claims 1 to 4, wherein the risk or scores of HCT from the candidate or actual donor to induce or not induce GVHD in a HCT recipient are recorded or stored on an electronic or computer readable medium.

86. The method of any of claims 1 to 4, further comprising creating a report of the risk or score of the HCT from the candidate donor to induce or to not induce graft vs. host disease (GVHD) in a HCT recipient.

87. The method of any of claims 1 to 4, wherein expression of the positive or negative predictor genes, or a housekeeping gene, is determined by RT-PCR.

88. A kit, comprising two or more primer pairs, wherein each primer pair is oppositely oriented to each other, wherein the first of the primer pairs hybridizes to RNA or cDNA produced from one of the positive or negative predictor genes and the second hybridizes to a housekeeping gene listed in Tables 1 (RNA 1538), 2, 2A, 2B (RNA 192), 3 and/or 12.

89. The kit of claim 88, comprising five or more primer pairs oppositely oriented to each other, wherein each of the five primer pairs hybridize to RNA or cDNA of the positive or negative predictor genes listed in Tables 1 (RNA 1538), 2, 2A (RNA 143), 2B (RNA 192), 3, 13 (SG175), 15 (SG128) or 18 (SG64), or a polymorphism thereof.

90. The kit of claim 88, comprising 10 or more primer pairs oppositely oriented to each other, wherein each of the 10 primer pairs hybridize to RNA or cDNA of the positive or negative predictor genes listed in Tables 1 (RNA 1538), 2, 2A (RNA 143), 2B (RNA 192), 3, 13 (SG175), 15 (SG128) or 18 (SG64), or a polymorphism thereof.

91. The kit of claim 88, comprising 20 or more primer pairs oppositely oriented to each other, wherein each of the 20 primer pairs hybridize to RNA or cDNA of the positive or negative predictor genes listed in Tables 1 (RNA 1538), 2, 2A (RNA 143), 2B (RNA 192), 3, 13 (SG175), 15 (SG128) or 18 (SG64), or a polymorphism thereof.

92. The kit of claim 88, further comprising a probe that hybridizes to a nucleic acid sequence amplified by one of the primer pairs.

93. The kit of claim 88, wherein each of the primer pairs are not affixed to a support or substrate.

94. A kit, comprising one or more nucleic acid probes, wherein said one or more probes hybridizes to RNA or cDNA of one or more of the positive or negative predictor genes listed in Tables 1 (RNA 1538), 2, 2A (RNA 143), 2B (RNA 192), 3, 13 (SG175), 15 (SG128) or 18 (SG64), or a polymorphism thereof.

95. The kit of claim 94, comprising five or more probes that hybridize to RNA or cDNA of five of the positive or negative predictor genes listed in Tables 1 (RNA 1538), 2, 2A (RNA 143), 2B (RNA 192), 3, 13 (SG175), 15 (SG128) or 18 (SG64), or a polymorphism thereof.

96. The kit of claim 94, comprising 10 or more probes that hybridize to RNA or cDNA of 10 of the positive or negative predictor genes listed in Tables 1 (RNA 1538), 2, 2A (RNA 143), 2B (RNA 192), 3, 13 (SG175), 15 (SG128) or 18 (SG64), or a polymorphism thereof.

97. The kit of claim 94, comprising 20 or more probes that hybridize to RNA or cDNA of 20 of the positive or negative predictor genes listed in Tables 1 (RNA 1538), 2, 2A (RNA 143), 2B (RNA 192), 3, 13 (SG175), 15 (SG128) or 18 (SG64), or a polymorphism thereof.

98. A database or organizational construct, comprising gene expression profiles of two or more positive or negative predictor genes from a plurality of actual or candidate HCT donors, wherein the two or more positive or negative predictor genes are listed in Tables 1 (RNA 1538), 2, 2A (RNA 143), 2B (RNA 192), 3, 13 (SG175), 15 (SG128) or 18 (SG64), or a polymorphism thereof, and wherein the database or organizational construct associates the gene expression profile with each of the actual or candidate HCT donors.

99. The database or organizational construct of claim 98, wherein the database comprises expression profiles of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more positive or negative predictor genes, and optionally one or more housekeeping genes.

100. The database or organizational construct of claim 98, wherein HCT from the actual or candidate donors at lower or higher risk of inducing graft vs. host disease (GVHD) in a HCT recipient are identified.

101. The database or organizational construct of claim 98, wherein expression of the positive or negative predictor genes is from a biological sample comprising actual or candidate donor CD4+ T cells or CD8+ T cells.

102. The database or organizational construct of claim 98, wherein the database is operatively linked to a processor, said processor comprising a data entry module or a data query module.

103. The database or organizational construct of claim 98, wherein one or more of the actual or candidate HCT donors are assigned a score based upon the probability or risk of their HCT to induce or not to induce graft vs. host disease (GVHD) in a HCT recipient.

104. An array of primers, comprising two or more primer pairs, wherein each primer pair is oppositely oriented to each other, wherein each of the primer pairs hybridize to RNA or cDNA produced from one of the positive or negative predictor genes listed in Tables 1 (RNA 1538), 2, 2A (RNA 143), 2B (RNA 192), 3, 13 (SG175), 15 (SG128) or 18 (SG64), or a polymorphism thereof, and wherein each primer pair is affixed to or contained in a support or substrate.

105. An array of probes, wherein each probe hybridizes to RNA or cDNA produced from a positive or negative predictor gene listed in Tables 1 (RNA 1538), 2, 2A (RNA 143), 2B (RNA 192), 3, 13 (SG175), 15 (SG128) or 18 (SG64), or a polymorphism thereof, and wherein each probe is affixed to or contained in a support or substrate.

106. The array of claim 104 or 105, further comprising a primer pair or probe that hybridizes to RNA or cDNA produced by a housekeeping gene.

107. The array of claim 104 or 105, wherein each primer pair or probe has a known position or address on the support or substrate.

108. The array of claim 104 or 105, wherein all of the primer pairs or probes hybridize to RNA or cDNA of the positive or negative predictor genes listed in Tables 1 (RNA 1538), 2, 2A (RNA 143), 2B (RNA 192), 3, 13 (SG175), 15 (SG128) or 18 (SG64), or a polymorphism thereof.

109. The array of claim 104 or 105, comprising primer pairs or probes that hybridize to RNA or cDNA of 5, 10, 20, 30 or more of the positive or negative predictor genes listed in Tables 1 (RNA 1538), 2, 2A, 2B (RNA 192) and/or 3, or a polymorphism thereof.

110. The array of claim 104 or 105, wherein the total primer pairs or probes comprising the array are less than 20,000, less than 15,000, less than 10,000, less than 5,000, less than 2,500, less than 2,000, less than 1,500, less than 1,000, less than 500, less than 400, less than 300, less than 200, less than 100, less than 50, or less than 25 primer pairs or probes.

111. The array of claim 104 or 105, wherein the support or substrate comprises a multi-well format.

112. The array of claim 104 or 105, wherein the support or substrate comprises a multi-well plate.

113. The array of claim 104, further comprising a probe that hybridizes to a nucleic acid sequence amplified by one of the primer pairs.

114. The method of any of claims 1 to 4, wherein the CD4+ T cells or CD8+ T cells comprise a biological sample.

115. The method of any of claims 72 to 77, the kit of claim 88 or 924 or the array of claim 104 or 105, wherein the oligo- or poly-nucleotide probe or primer has a length of about 5-10, 10-20, 20-30, 30-40, 40-50, 50-60, 60-70, 70-80, 90-100, 100-150, 150-200, 200-250, 250-300, 300-400, 400-500, 500-1000, or 1000-2000 nucleotides.
